(19) United States
(12) Patent Application Publication
(10) Pub. No.: US 2012/0157324 A1
(43) Pub. Date: Jun. 21, 2012

(54) METHYLATION BIOMARKERS AND METHODS OF USE

(75) Inventors: Paul M. Lizardi, Wallingford, CT (US); Sebastian Szpakowski, Silver Spring, MD (US); Min Chen, Dallas, TX (US); Jose Costa, Guilford, CT (US); Hongyu Zhao, Guilford, CT (US)

(73) Assignee: YALE UNIVERSITY, New Haven, CT (US)

(21) Appl. No.: 13/390,669

(22) PCT Filed: Aug. 17, 2010
(86) PCT No.: PCT/US2010/045788

§ 371 Date: Feb. 15, 2012

Related U.S. Application Data
(60) Provisional application No. 61/234,367, filed on Aug. 17, 2009.

(51) Int. Cl. C12Q 1/68; C40B 50/00; C40B 20/00; C07H 21/04; C07H 1/00

(52) U.S. Cl. 506/2; 435/6.11; 536/23.1; 536/25.3; 506/23

(57) ABSTRACT

Disclosed are methods and compositions of assessing one or more statuses of a subject. Also disclosed are methods and compositions of identifying status biomarkers associated with a status of a subject. Also disclosed are sets of one or more status biomarkers. Also disclosed are methods and compositions of producing status biomarker capture probes.

### CROSS-REFERENCE TO RELATED APPLICATIONS

This application claims benefit of U.S. Provisional Application No. 61/234,367, filed Aug. 17, 2009. Application No. 61/234,367, filed Aug. 17, 2009, is hereby incorporated herein by reference in its entirety.

### STATEMENT REGARDING FEDERALLY SPONSORED RESEARCH

This invention was made with government support under grant No. 1R21CA116079 from the National Institutes of Health (NIH). The government has certain rights in the invention.

### BACKGROUND

The DNA of most tumors has a reduced content of methylated cytosine residues. This so-called global “hypomethylation” affects primarily DNA sequences that belong to interspersed DNA repeats. In normal human tissues, DNA repeats are predominantly methylated, consistent with the requirement to maintain genomic stability by transcriptional silencing of retroelements whose potential deleterious functions include DNA mobilization as well as the facilitation of recombination events in somatic cells.

### BRIEF SUMMARY

Disclosed are methods and compositions of assessing one or more statuses of a subject. Also disclosed are methods and compositions of identifying status biomarkers associated with a status of a subject. Also disclosed are sets of one or more status biomarkers. Also disclosed are methods and compositions of producing status biomarker capture probes.

In some forms of the methods and compositions of assessing one or more statuses of a subject, the method can comprise, for example, determining the methylation state of one or more status biomarkers in the subject, and comparing one or more of the determined methylation states to one or more reference methylation states, wherein a difference, lack of a difference, or both in one or more of the determined methylation states and one or more of the reference methylation states indicates one or more statuses of the subject.

In some forms of the methods and compositions of identifying status biomarkers associated with a status of a subject, the method can comprise, for example, determining the methylation state of one or more status biomarkers in one or more DNA samples, wherein the DNA samples are from sources that are relevant to one or more specific statuses, and comparing one or more of the determined methylation states to one or more reference methylation states, wherein a difference in one or more of the determined methylation states and one or more of the reference methylation states indicates that the status biomarkers for which the difference in the methylation states is found is a status biomarker associated with one or more of the specific statuses.

In some forms, the methylation state can be determined by, for example, treating a DNA sample of the subject to differentiate methylated and unmethylated nucleotides, and detecting the level of methylated forms of the one or more status biomarkers in the treated DNA, detecting the level of unmethylated forms of the one or more status biomarkers in the treated DNA, or both, wherein the level of methylated forms of the status biomarkers, the level of unmethylated forms of the status biomarkers, or both indicates the methylation state of the status biomarkers.

In some forms, treating the DNA sample can be accomplished by, for example, incubating the DNA sample with one or more restriction endonucleases and amplifying the incubated DNA, wherein the restriction endonucleases are methylation-sensitive restriction endonucleases, wherein the level of the status biomarkers in the amplified DNA is lower when the status biomarkers have reduced methylation and the level of the status biomarkers in the amplified DNA is higher when the status biomarkers have increased methylation, wherein the level of the status biomarkers comprise the level of methylated forms of the one or more status biomarkers in the treated DNA, the level of unmethylated forms of the one or more status biomarkers in the treated DNA, or both.

In some forms, the restriction endonucleases can further comprise at least one methylation-dependent restriction endonuclease. In some forms, the restriction endonucleases can further comprise at least one methylation-independent restriction endonuclease. In some forms, the restriction endonucleases can comprise AciI and HhaI. In some forms, the restriction endonucleases can comprise McrBC. In some forms, incubating the DNA sample with one or more endonucleases can be accomplished by, for example, incubating different aliquots of the DNA sample with different restriction endonucleases. In some forms, amplifying the incubated DNA can be accomplished by, for example, multiple displacement amplification.

In some forms, treating the DNA sample can be accomplished by, for example, processing the DNA sample with sodium bisulfite.

In some forms, treating the DNA sample can be accomplished by, for example, fragmenting the DNA and separating methylated DNA from unmethylated DNA. In some forms, the DNA can be fragmented by, for example, nebularization, cleavage with a restriction endonuclease, sonication, or a combination. In some forms, methylated DNA can be separated from unmethylated DNA by, for example, binding methylated DNA with a specific binding molecule specific for methyl groups and separating the bound from the unbound DNA. In some forms, the specific binding molecule can comprise, for example, an antibody specific for 5-methyl cytosine, methyl-biding protein MBD1, methyl-biding protein MECP2, or a combination.

In some forms, treating the DNA sample can be accomplished by, for example, capturing status biomarker DNA fragments and sequencing the captured status biomarker DNA fragments, wherein the sequencing distinguishes cytosine from methylcytosine, wherein the level of methylcytosine indicates level of methylated forms of the status biomarkers. In some forms, the status biomarker DNA fragments can be captured by, for example, binding DNA fragments in the DNA sample to status biomarker probes attached to a support. In some forms, one or more of the status biomarker probes can specifically hybridize to one or more repetitive DNA sequences, wherein the one or more of the status biomarker probes comprises degenerate sequence representing a consensus sequence for a family of repetitive DNA sequences. In some forms, each of the status biomarker probes can specifically hybridize to one or more repetitive DNA sequences, wherein each of the status biomarker probes comprises degenerate sequence representing a consensus sequence for a family of repetitive DNA sequences. In some forms, the family of repetitive DNA sequences can be a family of repetitive DNA sequences listed in, for example, Table 1. In some forms, the one or more of the status biomarker probes can comprise different degenerate sequences each representing a consensus sequence for a different one of the families of repetitive DNA sequences listed in, for example, Table 1. In some forms, the one or more of the status biomarker probes can comprise at least 20 different degenerate sequences each representing a different consensus sequence for a different one of the families of repetitive DNA sequences listed in, for example, Table 1. In some forms, the support can comprise, for example, gel, a bead, a magnetic bead, a plate, a slide, a surface, or a microparticle. In some forms, DNA not captured can be separated from the captured status biomarker DNA fragments. In some forms, the sequencing can be a form of SMRT sequencing.

In some forms, the method can further comprise, after capturing status biomarker DNA fragments and prior to sequencing the captured status biomarker DNA fragments, releasing the captured status biomarker DNA fragments and recapturing the released status biomarker DNA fragments. In some forms, the status biomarker DNA fragments can be recaptured by binding DNA fragments in the DNA sample to secondary status biomarker probes attached to a support. In some forms, one or more of the status biomarker probes can specifically hybridize to one or more repetitive DNA sequences, wherein the one or more of the status biomarker probes comprises degenerate sequence representing a consensus sequence for a family of repetitive DNA sequences. In some forms, each of the status biomarker probes can specifically hybridize to one or more repetitive DNA sequences, wherein each of the status biomarker probes comprises degenerate sequence representing a consensus sequence for a family of repetitive DNA sequences. In some forms, the family of repetitive DNA sequences can be a family of repetitive DNA sequences listed in, for example, Table 16 and Table 17. In some forms, the family of repetitive DNA sequences can be a family of repetitive DNA sequences listed in Table 16 or 17. For example, the family of repetitive DNA sequences can be the AluY, AluSx, AluSp, AluSg, or AluSc family of repetitive DNA sequences. In some forms, the one or more of the status biomarker probes can comprise different degenerate sequences each representing a consensus sequence for a different one of the families of repetitive DNA sequences listed in, for example, Table 16 and Table 17. In some forms, the one or more of the status biomarker probes can comprise different degenerate sequences each representing a consensus sequence for a different one of the families of repetitive DNA sequences listed in Table 16 or 17, such as AluY, AluSx, AluSp, AluSg, or AluSc. In some forms, the support can comprise, for example, gel, a bead, a magnetic bead, a plate, a slide, a surface, or a microparticle. In some forms, DNA not recaptured can be separated from the recaptured status biomarker DNA fragments.

In some forms, detecting the level of the status biomarkers can be accomplished via, for example, an array of probes specific for the status biomarkers. In some forms, the array of probes can be, for example, a microarray.

In some forms, detecting the level of the status biomarkers can be accomplished via, for example, amplifying the processed DNA and determining the ratio of cytosine to thymidine in the amplified DNA and converting the ratio to the level of methylated forms of the status biomarkers. In some forms, the processed DNA can be amplified via, for example, PCR amplification of the status biomarkers using primers specific for the status biomarkers.

In some forms, detecting the level of the status biomarkers can be accomplished via, for example, PCR amplification of the status biomarkers using primers specific for the status biomarkers. In some forms, the PCR amplification can be quantitative PCR. In some forms, the PCR amplification can be nanoliter-microarray quantitative PCR.

In some forms, the level of the status biomarkers can be grouped into a plurality of status biomarker families, wherein the level of the status biomarkers in one or more of the families is analyzed, wherein the analyzed level of the status biomarkers in the one or more of the families indicates the methylation state of the status biomarkers in the family. In some forms, the analyzed level of the status biomarkers in one or more of the families can be the average of the levels of the individual status biomarkers in the family. In some forms, one or more of the status biomarker families each independently can consist of, for example, a single class of repetitive DNA element, a single subclass of repetitive DNA element, a single family of repetitive DNA element, a single subfamily of repetitive DNA element, or a combination. In some forms, the analyzed level of the status biomarkers in one or more of the families can be normalized to one or more of the reference methylation states. In some forms, the level of one or more of the status biomarkers can be normalized to one or more of the reference methylation states. In some forms, the level of one or more of the status biomarker families can be normalized to one or more of the reference methylation states. In some forms, the status biomarkers can be grouped according to one or more repetitive DNA sequences that the status biomarkers comprise, wherein each biomarker in each status biomarker family comprises one or more repetitive DNA sequences that belong to a single family of repetitive DNA sequences listed in, for example, Table 1.

In some forms, one or more of the one or more reference methylation states can be a normal methylation state. In some forms, the normal methylation state can be, for example, the methylation state of a healthy subject, the average of the methylation states of healthy subjects, or the average of the methylation states of a population of subjects. In some forms, one or more of the one or more reference methylation states can be, for example, the methylation state of the same subject at a different time, the methylation state of the same subject at an earlier time, the methylation state of the same subject at a later time, or the methylation state of one or more normal cells, tissues, organs, or a combination of the same subject. In some forms, one or more of the one or more reference methylation states can be the methylation state from non-tumor adjacent tissue. In some forms, one or more of the one or more reference methylation states can be a normal methylation state of a status biomarker family.

In some forms, the method can further comprise determining the genetic state of one or more status biomarkers by, for example, comparing one or more of the determined genetic states to one or more reference genetic states, wherein a difference, lack of a difference, or both in one or more of the determined genetic states and one or more of the reference genetic states indicates one or more statuses of the subject. In some forms, determining the genetic state of one or more status biomarkers can be determined in one or more of the DNA samples.

In some forms, the source of one or more of the DNA samples can be one or more tissues of the subject, organs of the subject, or both. In some forms, the source of one or more of the DNA samples can be a tissue or organ of the subject. In some forms, the source of one or more of the DNA samples can be one or more cells of the subject. In some forms, the source of one or more of the DNA samples can be one or more cells, tissue, skin, lung, head, neck, prostate, breast, ovary, brain, liver, stomach, intestine, kidney, testicle, cervix, uterus, spleen, bone, throat, esophagus, muscle, bodily fluids, blood, urine, semen, lymphatic fluid, cerebrospinal fluid, amniotic fluid, biological samples, tissue culture cells, buccal swabs, mouthwash, stool, tissues slices, biopsy aspiration, or a combination.

In some forms, the subject can be assessed for the status of wellness, level of health, risk to wellness, risk to level of health, or a combination. In some forms, the subject can be assessed for the status of the genome. In some forms, the subject can be assessed for the status of aging, risk of aging, or both. In some forms, the subject can be assessed for the status of cancer, risk of cancer, or both. In some forms, the subject can be assessed for the status of stress response. In some forms, the subject can be assessed for the status of diabetes, risk of diabetes, or both. In some forms, the subject can be assessed for the status of heart disease, risk of heart disease, or both. In some forms, the subject can be assessed for the status of genomic instability. In some forms, the subject can be assessed for the status of tumor burden. In some forms, the subject can be assessed for the status of response to treatment.

In some forms, the subject can be assessed for a change in one or more statuses. In some forms, the change in one or more of the one or more statuses can be assessed compared to an earlier assessment. In some forms, the earlier assessment can have been made at, for example, an earlier time, prior to diagnosis of a disease or condition, prior to a treatment, following diagnosis of a disease or condition, following treatment, or a combination. In some forms, the change in one or more of the one or more statuses can be assessed following the passage of time, prior to diagnosis of a disease or condition, prior to a treatment, following diagnosis of a disease or condition, following treatment, or a combination. In some forms, assessing the subject can comprise assessing one or more tissues of the subject, organs of the subject, or both. In some forms, assessing the subject can comprise assessing a tissue or organ of the subject. In some forms, assessing the subject can comprise assessing one or more cells of the subject.

In some forms, the status biomarkers can comprise nucleic acid sequences in the genome of the species to which the subject belongs. In some forms of the sets of one or more status biomarkers the status biomarkers can comprise, for example, nucleic acid sequences in a genome. In some forms, the nucleic acid sequences can be in proximity to CpG islands or islets, wherein the CpG islands or islets comprise nucleic acid regions greater than 100 nucleotides in length that contain a minimum of 5 CpG residues and have a ratio of CG content to GC content greater than 0.3. In some forms, the CpG islands or islets can comprise nucleic acid regions greater than 200 nucleotides in length. In some forms, the CpG islands or islets can comprise nucleic acid regions greater than 300 nucleotides in length. In some forms, the nucleic acid regions can have a ratio of CG content to GC content greater than 0.4. In some forms, the nucleic acid regions can have a ratio of CG content to GC content greater than 0.5. In some forms, the status biomarkers can be in proximity to CpG islands or islets when they are within 1200 bases of a CpG island or islet.

In some forms, one or more of the status biomarkers can overlap with all or part of a CpG island or islet. In some forms, the one or more of the status biomarkers can comprise a probe binding site, wherein the probe binding site of the one or more of the status biomarkers is specific for a probe. In some forms, one or more of the probes can be specific for a repetitive DNA sequence locus, wherein the repetitive DNA sequence locus comprises one or more repetitive DNA sequences, wherein independently for each of the one or more of the probes one or more of the repetitive DNA sequences belongs to a family of repetitive DNA sequences listed in, for example, Table 1. In some forms, each probe can be specific for a repetitive DNA sequence locus, wherein independently for each probe one or more of the repetitive DNA sequences belongs to a family of repetitive DNA sequences listed in, for example, Table 1. In some forms, one or more of the probes can be specific for a repetitive DNA sequence locus, wherein the repetitive DNA sequence locus comprises one or more repetitive DNA sequences, wherein for one or more of the probes one or more of the repetitive DNA sequences is an interspersed repeat element. In some forms, each probe can be specific for a repetitive DNA sequence locus, wherein for each probe one or more of the repetitive DNA sequences is an interspersed repeat element.

In some forms, one or more of the status biomarkers can comprise a PCR amplicon. In some forms, the PCR amplicon of each of the one or more of the status biomarkers can be defined by a first primer specific for a single one of the status biomarkers and a second primer. In some forms, the PCR amplicon of each of the one or more of the status biomarkers can be defined by the same first primer specific for a first type of repetitive DNA sequence and a second primer, wherein the second primer is specific for a second type of repetitive DNA sequence, wherein the second primer is the same for some and different for some of the one or more of the status biomarkers. In some forms, the first primer can be specific for one of the families of repetitive DNA sequences listed in Table 16 or 17, wherein independently for each of the one or more of the status biomarkers the second primer is specific for a family of repetitive DNA sequences listed in, for example, Table 1.

In some forms, one or more of the status biomarkers can comprise one or more repetitive DNA sequences, wherein independently for each of the one or more of the status biomarkers that comprise repetitive DNA sequences one or more of the repetitive DNA sequences belongs to a family of repetitive DNA sequences listed in, for example, Table 1. In some forms, each status biomarker can comprise a repetitive DNA sequence, wherein independently for each of the status biomarkers the repetitive DNA sequence belongs to a family of repetitive DNA sequences listed in, for example, Table 1. In some forms, one or more of the status biomarkers can comprise one or more repetitive DNA sequences, wherein for one or more of the status biomarkers that comprise repetitive DNA sequences one or more of the repetitive DNA sequences is an interspersed repeat element. In some forms, each status biomarker can comprise a repetitive DNA sequence, wherein for each status biomarker the repetitive DNA sequence is an interspersed repeat element.

In some forms, the methylation state of more than 100 biomarkers is determined. In some forms, the methylation state of more than 1000 biomarkers can be determined. In some forms, the methylation state of more than 10,000 biomarkers can be determined. In some forms, the methylation state of more than 100,000 biomarkers can be determined. In some forms, the methylation state of more than 200,000 biomarkers can be determined. In some forms, the status biomarkers can comprise a set of status biomarkers. In some forms, the set can comprise more than 100 status biomarkers. In some forms, the set can comprise more than 1000 status biomarkers. In some forms, the set can comprise more than 10,000 status biomarkers. In some forms, the set can comprise more than 100,000 status biomarkers. In some forms, the set can comprise more than 200,000 status biomarkers.

In some forms, a plurality of the biomarkers can independently belong to one or more status biomarker families, wherein each biomarker in each status biomarker family comprises one or more repetitive DNA sequences that belong to a single family of repetitive DNA sequences listed in, for example, Table 1. In some forms, a plurality of biomarkers can independently belong to two or more status biomarker families. In some forms, a plurality of biomarkers can independently belong to three or more status biomarker families. In some forms, a plurality of biomarkers can independently belong to four or more status biomarker families. In some forms, a plurality of biomarkers can independently belong to five or more status biomarker families. In some forms, a plurality of biomarkers can independently belong to ten or more status biomarker families. In some forms, a plurality of biomarkers can independently belong to twenty or more status biomarker families.

In some forms, 100 or more biomarkers can belong to one or more of the status biomarker families. In some forms, 100 or more biomarkers can belong to each of the status biomarker families. In some forms, 200 or more biomarkers can belong to one or more of the status biomarker families. In some forms, 200 or more biomarkers can belong to each of the status biomarker families. In some forms, 300 or more biomarkers can belong to one or more of the status biomarker families. In some forms, 300 or more biomarkers can belong to each of the status biomarker families. In some forms, 400 or more biomarkers can belong to one or more of the status biomarker families. In some forms, the 400 or more biomarkers can belong to each of the status biomarker families.

In some forms, the status biomarkers can comprise a set of status biomarkers. In some forms, the members of the set of status biomarkers can be status biomarkers that indicate the status of one or more specific statuses. In some forms, the one or more specific statuses can comprise, for example, wellness, level of health, risk to wellness, risk to level of health, status of the genome, genomic instability, aging, risk of aging, cancer, risk of cancer, head and neck cancer, risk of head and neck cancer, breast cancer, risk of breast cancer, lung cancer, risk of lung cancer, prostate cancer, risk of prostate cancer, colon cancer, risk of colon cancer, esophageal cancer, risk of esophageal cancer, ovarian cancer, risk of ovarian cancer, liver cancer, risk of liver cancer, pancreatic cancer, risk of pancreatic cancer, skin cancer, risk of skin cancer, melanoma, risk of melanoma, lymphoma, risk of lymphoma, leukemia, risk of leukemia, cervical cancer, risk of cervical cancer, cervical dysplasia, risk of cervical dysplasia, cervical intraepithelial neoplasia, risk of cervical intraepithelial neoplasia, tumor burden, stress response, diabetes, risk of diabetes, heart disease, risk of heart disease, and/or response to treatment.

In some forms, the one or more specific statuses can comprise the presence of a disease or condition. In some forms, the one or more specific statuses can comprise, for example, a lack of wellness, low level of health, risk to wellness, risk to level of health, poor status of the genome, genomic instability, aging, risk of aging, cancer, risk of cancer, head and neck cancer, risk of head and neck cancer, breast cancer, risk of breast cancer, lung cancer, risk of lung cancer, prostate cancer, risk of prostate cancer, colon cancer, risk of colon cancer, esophageal cancer, risk of esophageal cancer, ovarian cancer, risk of ovarian cancer, liver cancer, risk of liver cancer, pancreatic cancer, risk of pancreatic cancer, skin cancer, risk of skin cancer, melanoma, risk of melanoma, lymphoma, risk of lymphoma, leukemia, risk of leukemia, cervical cancer, risk of cervical cancer, cervical dysplasia, risk of cervical dysplasia, cervical intraepithelial neoplasia, risk of cervical intraepithelial neoplasia, tumor burden, stress response, diabetes, risk of diabetes, heart disease, and/or risk of heart disease.

In some forms of the methods and compositions of producing status biomarker capture probes, the method can comprise, for example, selecting a subset of repetitive DNA sequence loci from a set of repetitive DNA sequence loci, generating a set of status biomarker capture probe sequences, and synthesizing one or more status biomarker capture probes. In some forms, the repetitive DNA sequence loci in the set of repetitive DNA sequence loci can belong to a single one of the families of repetitive DNA sequence listed in, for example, Table 1, wherein the subset of repetitive DNA sequence loci can be selected by identifying those repetitive DNA sequence loci that comprise a repetitive DNA sequence belonging to one of the families of repetitive DNA sequences listed in, for example, Table 16 and Table 17.

In some forms, each status biomarker capture probe sequence in the set can have a length of 50 bases or more, wherein each status biomarker capture probe represented in the set of status biomarker capture probe sequences can hybridize to at least 5% of the repetitive DNA sequence loci in the selected subset of repetitive DNA sequence loci. In some forms, each status biomarker capture probe can have the sequence of one of the status biomarker capture probe sequences.

In some forms, the repetitive DNA sequence loci in the set of repetitive DNA sequence loci can belong to a single one of the families of repetitive DNA sequence LTR54B, MER11B, MER34B, LTR56, THE1B, HERV9, LTR14C, HERVFH21, LTR6B, LTR46, MLT1D, MER67D, HERVK11, LTR10B, HERVK22, MER6, MER66C, MLT1G1, MER4D, and MLTD2. In some forms, the repetitive DNA sequence in the subset of repetitive DNA sequence loci can belong to one of the families of repetitive DNA sequences listed in Table 16 or 17, such as AluY, AluSx, AluSp, AluSg, AluSc, LTR9, or LTR9B.

In some forms, the method can further comprise selecting one or more additional subsets of repetitive DNA sequence loci each from a different additional set of repetitive DNA sequence loci, generating one or more additional sets of status biomarker capture probe sequences each based on one of the one or more additional subsets, and synthesizing one or more additional status biomarker capture probes, wherein each additional status biomarker capture probe has the sequence of one of the additional status biomarker capture probe sequences. In some forms, the repetitive DNA sequence loci in each additional set of repetitive DNA sequence loci can independently belong to a different single one of the families of repetitive DNA sequence listed in, for example, Table 1, wherein the repetitive DNA sequence loci in the set of repetitive DNA sequence loci and in each additional set of repetitive DNA sequence loci belong to different families of repetitive DNA sequence.

In some forms, the repetitive DNA sequence loci in the each additional set of repetitive DNA sequence loci can independently belong to a single one of the families of repetitive DNA sequence LTR54B, MER11B, MER34B, LTR56, THE1B, HERV9, LTR14C, HERVFH21, LTR6B, LTR46, MLT1D, MER67D, HERVK11, LTR10B, HERVK22, MER6, MER66C, MLT1G1, MER4D, and MLTD2. In some forms, each status biomarker capture probe sequence in the set can have a length of 100 bases or more. In some forms, each status biomarker capture probe represented in the set of status biomarker capture probe sequences can hybridize to at least 10% of the repetitive DNA sequence loci in the selected subset of repetitive DNA sequence loci. In some forms, the set of status biomarker capture probe sequences can comprise from 1 to 100 status biomarker probe capture sequences. In some forms, the set of status biomarker capture probe sequences can comprise from 5 to 100 status biomarker probe capture sequences. In some forms, the set of status biomarker capture probe sequences can comprise from 10 to 100 status biomarker probe capture sequences. In some forms, one or more of the additional sets of status biomarker capture probe sequences each can comprise from 1 to 100 status biomarker probe capture sequences. In some forms, the one or more additional sets of status biomarker capture probe sequences each can comprise from 5 to 100 status biomarker probe capture sequences. In some forms, the one or more additional sets of status biomarker capture probe sequences each can comprise from 10 to 100 status biomarker probe capture sequences.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 is divided into 3 sections, with each of the sections summarizing information about a subset of experiments: 10 Normal, 17 Non-Tumor Adjacent or 33 Tumor. Initially, within each experiment, a subset of probes reporting on a particular group of repetitive elements were averaged to present a single methylation level per repetitive element group in an experiment. Subsequently the mean methylation levels from a subset of experiments were pooled to calculate the median value plotted in the figure. The repetitive element categories are indicated on the left side of the plot. The number in parenthesis next to each name indicates how many probe measurements were averaged in each of the experiments. The categories in the shaded boxes represent the results of in-silico PCR amplification performed using the LINE-element-amplifying primers as described in the literature (Choi et al. Carcinogenesis 2009; Woloszynska-Read et al. Clinical Cancer Research 2008; Rago et al. Cancer research 2007; Chalitchagorn et al. Oncogene 2004; Yang et al. Nucleic Acids Res 2004; Sunami et al. Ann NY Acad Sci 2008). The in-silico PCR was conducted as follows: first primer sequences from the literature were aligned to the genome using FASTA. Subsequently the FASTA outputs for two complementary primers were parsed and filtered so that only the alignments separated by 50-1500 bases were reported as the in-silico PCR amplicons. The coordinates of the amplicons were used to query the RepBase database of repetitive elements to classify what kind of repetitive elements were amplified. Given that the primers allow for amplification of various groups of repetitive elements, the relative amount of each was recorded for use in the subsequent step. The values plotted are thus based on an average of the methylation levels of several repetitive elements group in an experiment, weighed according to the relative composition of the in-silico PCR result. These weighted averages are subsequently pooled per experiment subset to plot the subset's median. The arrows point to the values that were generated using the in-silico PCR reaction. The order or repetitive element categories within each of the 3 sections of this figure is constant, and based on the information content of the methylation levels of given repeat element category in normal and tumor experiments. The information content is calculated using Shannon Entropy measure, and the categories are ordered so that the most informative is on the bottom. Furthermore, the 5 most informative categories of repetitive elements are highlighted. The remaining less-informative categories are plotted in gray.

FIG. 2 represents a Random Forest List of Category importance based on Mean Decreased Accuracy (left panel) and Mean Decreased Gini (right panel). The top 30 categories shown in the plot were selected from a list of 139,379 variables including gene probes, unique probes and repetitive element categories.

FIG. 3 shows a multi-dimensional scaling plot of proximity of the experiments based on the random forest classification. 1 represents Normal experiments, 2—Non-Tumor Adjacent experiments, 3 represents a Tumor experiment. The distance between any 2 experiments represents the frequency of classification into the same category based on the “forest” of 45,000 classification trees. 139,379 categories.

FIG. 4 shows Receiver-Operator Curves for Margin and Normal experiments (left panel) Tumor and Non-Tumor Adjacent experiments (right panel). Out-Of-Bag (OOB) cross validation results are shown. 139,379 categories.

FIGS. 5A-5F show a simplified diagram summarizing the steps of an example of the disclosed methods Example 3). (A) DNA is first acquired from a tissue material (B) the DNA is split into two equal aliquots (C) each of them is then digested with methylation sensitive or dependent enzymes (D) the DNA is then amplified (E) labeled and (F) hybridized to a microarray.

FIG. 6 represents examples of probe design and microarray response for two probes near repetitive elements. The figure shows the genomic context of a repetitive element, the locations of probes, CpG islands, other repeats, potential enzyme cuts as well as outcomes from 6 methylation experiments. The top part was generated using the UCSC genome browser. The top 2 boxes of both 6A and 6B are normal samples, the middle two boxes are non-tumor adjacent samples and the bottom two boxes are tumor samples and each of the six boxes corresponds to a single methylation experiment. The text underneath provides a summary of a region using ASCII characters (generated using a tool ASCIIMap). The 6 ASCIIMap tracks show the location of the probe (∘ and highlighted with an arrow) and ˜700 bases up- and downstream (:) which together form a region where the probe's signal is coming from. The location of a CpG island is marked underneath (*) as are the locations of repetitive elements in the area (1—Line1, 2—Line2, T—LTR(MaLR), S—SINE, =—other, A—Alu, etc.). The vertical bars (|) indicate the presence of an enzyme recognition site for AciI, HHaI and McrBC enzymes respectively. The resolution of 1 character is about 100 nucleotides. FIG. 6A shows L1PA3, the total region shown is approximately 16 kb wide and FIG. 6B shows THE1C, the total region shown is approximately 11 kb wide.

FIG. 7 shows the four sections of the plot indicate 4 distinct classes of tissue types used for methylation profiling: Normals (10 experiments), non-tumor adjacent (17 experiments), Tumors (33 Experiments) and Sperm (3 replicate experiments). Each of the four sections contains the methylation levels of the same 13 categories of repetitive elements. Per category, the values are summarized using a box-and-whisker plot. A line within each box indicates the median value. Box boundaries are drawn based on 1^{st} and 3^{rd} quartiles. The dashed lines extending from the box indicate the extreme values of the distribution. Outliers, if any, are indicated by a circle. The classes and families of repetitive elements are indicated on the left of the box-and-whisker segment. The number in parenthesis next to the category description indicates the number of probes corresponding to the number of repetitive elements uniquely probed in the genome. The order of categories is constant in all four of the subsections. It was established based on the extent of variation in the plotted distributions using Shannon entropy information content metric. Only Normal and Tumor experiments were used to calculate the Shannon's Information metric. For a more detailed explanation see ‘Plotting the data’ section in Example 2. FIG. 7A shows the distribution of average methylation levels per category. In each of the 4 subsections of the plot the pertinent experiments contributed an average methylation level for all probes in proximity of a specific class of repetitive element. FIG. 7B shows the same as 7A except this time every experiment is normalized using an average of all tumor-adjacent experiments.

FIG. 8 shows the distributions of average methylation levels per lineages of MaLR (Smit, 1993) in subsets of experiments. In each of the 4 subsections of the plot, the pertinent experiments contributed an average methylation level for all probes in proximity of a specific class of repetitive element indicated on the left. The values are summarized using a box-and-whisker plot. A star next to a name indicates that it is primate-specific, and the estimated time of its origin in the genome is less than 60 million years ago (MYA). Table 10 contains detailed information about the ages of each of the subfamilies. (Pace and Feschotte, 2007; Khan et al., 2006; Batzer and Deininger, 2002; Kapitonov and Jurka, 1996).

FIG. 9 shows the average methylation levels of repetitive element categories per experiment. Numbers in parenthesis indicate how many probes were averaged per experiment (See also the ‘Plotting the Data’ section in Example 2)

FIGS. 10A and 10B show the ordering within plots. Per-experiment average methylation levels of the most informative subset of L1P and the least informative probes near DNA transposons and AluSq regions. (A) Experiments are not ordered. The dotted line indicates the average values of significant L1P probes in normal, non-tumor adjacent, tumor and sperm experiments (from top to bottom). (B) Experiments ordered within their groups based on L1P-mean(AluSq+DNA) probe values. The dotted line indicates the average values of significant L1P probes in normal, non-tumor adjacent, tumor and sperm experiments (from top to bottom).

FIGS. 11A and 11B represent example of a per-experiment plot showing average methylation levels of 4 categories of genomic compartments and per-category plot showing distributions of average methylation levels of 4 categories of genomic compartments, respectively. See the ‘Plotting the Data’ section in Example 2.

FIGS. 12A-12D characterize the genomic context of the repetitive element family (bin plot). The sub-plots characterize all repetitive elements of a particular class in the human genome. The bins of plot A summarize the distribution of CpG counts in all sequences of all repetitive elements from a given lineage and 1,500 bases up˜ and downstream from the repeat in 100 base increments per bin. The distribution of CpG in the repeat bin and external bins are presented in the form of a standard box and whisker plot, where the thick line inside the box indicates a median, the box is drawn around 25^{th} and 75^{th} percentiles, and the outliers are indicated as dots. Plots B and C keep the binding structure of the sequence as in plot A, and show the average number of potential enzyme cuts among all the sequences per bin normalized to the size of the bin. Gray lines indicate the standard deviation. Plot D is pertinent to the central bin of Plot A, it shows the distribution of sizes of all genomic repeats of a given family which were included in the central bin of plot A.

FIG. 13 shows the genomic organization of a biomarker probe LOCUS comprising a HUERS-P3 repetitive element, and two adjacent repetitive elements. The gene identifiers, RepeatMasker and Scale information in the figure were generated by the UCSC Genome Browser. The probe coordinates are chr17:73,126,561-73,126,611, and this position is indicated by a vertical arrow at the top of the figure. The locus is in the vicinity of the CD300 antigen-like family member B (BC028091, CD300LB). The annotation of Repeating Elements by RepeatMasker shows a HUERS-P3 element (grey bars), interrupted by an AluY sequence (black bar). The HUERS-P3 element is flanked on the left side by an LTR9B sequence (dark grey bar). The two large horizontal arrows near the bottom of the figure indicate the boundaries of the locus, which comprises approximately 1400 bases of genomic DNA. A CpG island is located exactly in the center of the locus, but is not shown in the figure.

### DETAILED DESCRIPTION

The disclosed method and compositions may be understood more readily by reference to the following detailed description of particular embodiments and the Example included therein and to the Figures and their previous and following description.

It has been discovered that the methylation status and/or level of certain loci in genomes can be used to assess and determine the status of subjects, tissues, and cells. For example, it has been discovered that the methylation status and/or level of certain repetitive DNA sequence loci and families of repetitive DNA sequence loci can distinguish the presence, absence, and/or risk or progress toward a variety of diseases and conditions.

The DNA of most tumors has a reduced content of methylated cytosine residues. This so-called global “hypomethylation” affects primarily DNA sequences that belong to interspersed DNA repeats. In normal human tissues, DNA repeats are predominantly methylated, consistent with the requirement to maintain genomic stability by transcriptional silencing of retroelements whose potential deleterious functions include DNA mobilization as well as the facilitation of recombination events in somatic cells. There have been a considerable number of reports of transcriptional activation of retrotransposons in the context of loss of DNA methylation. Expression of human endogenous retroviruses (HERVs) has been detected in breast cancer (Wang-Johanning et al., 2001), ovarian cancer (Menendez et al., 2004, Wang-Johanning et al., 2007), leukemia cell lines, (Patzke et al., 2002), urothelial and renal cell carcinomas (Florl et al., 1999). Increased transcriptional expression of HERV-K has been reported in teratocarcinoma (Löwer et al., 1984; Herbst et al., 1998), breast cancer cells and adjacent tissues (Wang-Johanning et al., 2003, Golan et al., 2008), and in melanoma (Muster et al., 2003; Büscher et al., 2006, Serafino et al., 2009). Stauffer et al. (2004) used massively parallel signature sequencing (MPSS) to define the number and type of transcripts of endogenous retroviruses of the LTR family in various cancers. This study reported that HERV-H, a relatively young retrotransposon, was expressed in cancers of the intestine, bone marrow, bladder and cervix, and was more highly expressed than the other families in cancers of the stomach, colon and prostate. Recently Alves et al. (2008) have reported that a specific HERVH element present in the X chromosome is selectively transcribed in 60% of colon cancers, and in a high proportion of metastatic colon cancers. There is evidence for context-specific induction of LINE-1 transcription during oxidative stress (Teneng et al., 2007). In a relatively large study of squamous head and neck carcinomas, Smith et al. (2007) reported that the DNA methylation level of LINE-1 elements was significantly reduced, and correlated with environmental insults such as alcohol use and smoking, as well as tumor stage.

Disclosed are methods and compositions of assessing one or more statuses of a subject. Also disclosed are methods and compositions of identifying status biomarkers associated with a status of a subject. Also disclosed are sets of one or more status biomarkers. Also disclosed are methods and compositions of producing status biomarker capture probes.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

### Materials

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a status biomarker is disclosed and discussed and a number of modifications that can be made to a number of molecules including the status biomarker are discussed, each and every combination and permutation of status biomarker and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### A. Status Biomarkers

Status biomarkers as used herein refer to nucleic acid sequences in a genome the methylation levels of which can be used to assess the status of a subject and/or one or more diseases, conditions, and/or states in a subject. Status biomarkers also include groups of such nucleic acid sequences, in the case of collective status biomarkers. Example 2 provides an example of identification of biomarkers that can be used to identify status biomarkers and all of the examples provide examples of how to identify status biomarkers and use status biomarkers for assessing the status of subjects and samples. Biomarkers from which status biomarkers are selected can be referred to as prospective status biomarkers.

Useful nucleic acid sequences for use as status biomarkers and nucleic acid sequences from which status biomarkers can be selected can include CpG islands or CpG islets and a unique sequence in proximity to a CpG island or CpG islet. Thus, status biomarkers and prospective status biomarkers can be loci having a unique sequence in proximity to a CpG island or CpG islet. CpG islands and CpG islets are described below and elsewhere herein. Proximity to a CpG island or CpG islet is described below and elsewhere herein. By unique sequence, in the context of status biomarkers, is meant a sequence of sufficient length and having a nucleotide sequence distinctive enough to be uniquely in the genome identified by a probe. For example, nucleic acid sequences of or at least 15, 20, 25, 30, 35, 40, 45, 50, 55, 60, 65, 70, 75, 80, 85, 90, 95, or 100 nucleotides in length can be used as unique sequences. Unique sequences can be identified by, for example, analysis of a genome sequence or by analysis of probe hybridization. The examples of selection of unique sequences herein make use of analysis of the human genome sequence. Status biomarkers are referred to herein by different terms such as variables, classifiers, and category classifiers.

In some forms of the sets of one or more status biomarkers the status biomarkers can comprise, for example, nucleic acid sequences in a genome. In some forms, the status biomarkers can comprise nucleic acid sequences in the genome of the species to which the subject belongs. In some forms, the nucleic acid sequences can be in proximity to CpG islands or islets. CpG islands and CpG islets are one significant location of DNA methylation that can affect gene expression. Example 2 describes the criteria used for selecting CpG islands and CpG islets, which was more lax than standard selection criteria. The CpG islands or islets can comprise nucleic acid regions of or greater than, for example, 20, 30, 40, 40, 60, 70, 80, 90, 100, 120, 140, 160, 180, 200, 220, 240, 260, 280, 300, 350, 400, or 500 nucleotides in length that contain a minimum of 5, 6, 7, 8, 9, 10, 11, or 12 CpG residues. The CpG islands and islets can have a ratio of CG content to GC content of or greater than, for example, 0.2, 0.3, 0.35, 0.38, 0.4, 0.40, 0.42, 0.43, 0.44, 0.45, 0.46, 0.47, 0.48, 0.49, 0.5, 0.50, 0.51, 0.52, 0.53, 0.54, 0.55, 0.56, 0.58, 0.59, 0.6, 0.60, 0.62, 0.65, 0.7, or 0.8. The sequence(s) that define the status biomarkers can be considered to be in proximity to CpG islands or islets when they are within 800, 850, 900, 950, 1000, 1050, 1100, 1150, 1200, 1250, 1300, 1350, 1400, 1450, 1500, 1600, 1700, 1800, 1900, or 2000 bases of a CpG island or islet.

569 repetitive DNA sequence families were identified from among the loci identified as CpG island- or CpG islet-containing loci as described in Example 2. Table 18 is a list of these repetitive DNA sequence families. Among the 569 repetitive element families comprising the full set of repetitive DNA sequence status biomarkers, a subset of 138 was identified that are most effective as classifiers. This subset was generated by merging the top 75 categories identified by a Random Forest analysis with another 75 categories that were the best performers using a Support Vector Machine classifier. This produced the list of Top 138 status biomarkers (Table 1). Each of these families represents multiple repetitive DNA sequence loci. Selected loci belonging to these families can be probed via unique sequences in the loci. Useful loci for the Top 138 families are specifically identified in Table 15 by listing of start and ending coordinates of example probe sequences in the loci. The loci identified by these probe sequences can be assessed, probed, detected, etc. according to the disclosed methods. The probe sequences identified in Table 15 are only examples of probe sequences that can be used to detect and assess the identified loci.

In some forms, one or more of the status biomarkers can overlap with all or part of a CpG island or islet. In some forms, the one or more of the status biomarkers can comprise a probe binding site, wherein the probe binding site of the one or more of the status biomarkers is specific for a probe. Probe binding sites can be, for example, all or a portion of a unique sequence in the status biomarker. In some forms, one or more of the probes can be specific for a repetitive DNA sequence locus, wherein the repetitive DNA sequence locus comprises one or more repetitive DNA sequences, wherein independently for each of the one or more of the probes one or more of the repetitive DNA sequences belongs to a family of repetitive DNA sequences listed in, for example, Table 1.

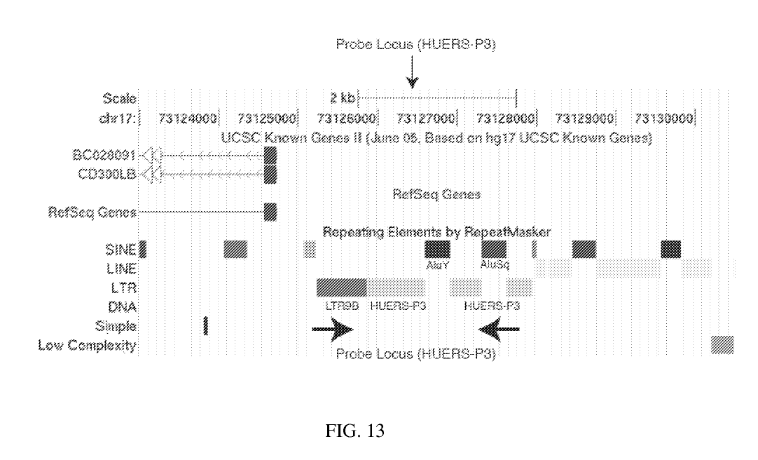

A repetitive DNA sequence is a DNA sequence that is repeated numerous times in a genome. Repetitive DNA sequences can also be referred to as repetitive DNA elements, repetitive sequences, repetitive elements, and repetitive DNA sequence elements. Repetitive DNA sequences can be repeated in different patterns in the genome, such as interspersed repetitive DNA sequences and tandem repetitive DNA sequences. A repetitive DNA sequence locus refers to a locus that includes one or more repetitive DNA sequences. An example of a repetitive DNA sequence locus is shown in FIG. 13. Repetitive DNA sequences have been classified into different families, sub-families, classes, subclasses, etc. of repetitive DNA elements. Although different such groups of repetitive DNA sequences can have different meanings, for convenience, all such groups and classifications are referred to herein as families or groups. Such reference should be considered to refer to all the different types of groups and classifications unless the context clearly indicates otherwise. Repetitive DNA sequence loci that comprise a given repetitive DNA sequence can be said to belong to the repetitive DNA sequence. Repetitive DNA sequence loci that comprise a repetitive DNA sequence that belongs to a given repetitive DNA sequence family can be said to belong to the repetitive DNA sequence family. In some forms, each probe can be specific for a repetitive DNA sequence locus, wherein independently for each probe one or more of the repetitive DNA sequences belongs to a family of repetitive DNA sequences such as the repetitive DNA sequence families listed in, for example, Table 1, Table 12, or Table 13.

In some forms, one or more of the probes can be specific for a repetitive DNA sequence locus, wherein the repetitive DNA sequence locus comprises one or more repetitive DNA sequences, wherein for one or more of the probes one or more of the repetitive DNA sequences is an interspersed repeat element. In some forms, each probe can be specific for a repetitive DNA sequence locus, wherein for each probe one or more of the repetitive DNA sequences is an interspersed repeat element.

In some forms, one or more of the status biomarkers can comprise a PCR amplicon. A PCR amplicon is a region of nucleic acid including and between the binding sites of PCR primers. PCR amplicons can be said to be defined by the binding sites of the primers and by the primers themselves. In some forms, the PCR amplicon of each of the one or more of the status biomarkers can be defined by a first primer specific for a single one of the status biomarkers and a second primer. A primer specific for a status biomarker refers to a primer that can bind to a sequence in, and prime replication of, the status biomarker. A primer specific for a repetitive DNA sequence refers to a primer that can bind to a sequence in, and prime replication of, the repetitive DNA sequence. In some forms, the PCR amplicon of each of the one or more of the status biomarkers can be defined by the same first primer specific for a first type of repetitive DNA sequence and a second primer, wherein the second primer is specific for a second type of repetitive DNA sequence, wherein the second primer is the same for some and different for some of the one or more of the status biomarkers. In some forms, the first primer can be specific for one of the families of repetitive DNA sequences listed in Table 16 or 17, wherein independently for each of the one or more of the status biomarkers the second primer is specific for a family of repetitive DNA sequences such as the repetitive DNA sequence families listed in, for example, Table 1, Table 12, or Table 13. A primer specific for a family of repetitive DNA sequence refers to a primer that can bind to a sequence in, and prime replication of, one or more repetitive DNA sequences in the family of repetitive DNA sequences.

In some forms, one or more of the status biomarkers can comprise one or more repetitive DNA sequences, wherein independently for each of the one or more of the status biomarkers that comprise repetitive DNA sequences one or more of the repetitive DNA sequences belongs to a family of repetitive DNA sequences such as the repetitive DNA sequence families listed in, for example, Table 1, Table 12, or Table 13. By independently is meant that, for each component in the group referred to, the specific identity of each component can be the same or different from the specific identity of any other of the components in the group. For example, in the group of status biomarkers above each different status biomarker can comprise the same or a different repetitive DNA sequence as any of the other status biomarkers in the group. In some forms, each status biomarker can comprise a repetitive DNA sequence, wherein independently for each of the status biomarkers the repetitive DNA sequence belongs to a family of repetitive DNA sequences such as the repetitive DNA sequence families listed in, for example, Table 1, Table 12, or Table 13. In some forms, one or more of the status biomarkers can comprise one or more repetitive DNA sequences, wherein for one or more of the status biomarkers that comprise repetitive DNA sequences one or more of the repetitive DNA sequences is an interspersed repeat element. In some forms, each status biomarker can comprise a repetitive DNA sequence, wherein for each status biomarker the repetitive DNA sequence is an interspersed repeat element.

The disclosed components, such as status biomarker loci, repetitive DNA sequences, repetitive DNA loci, biomarkers, status biomarkers prospective status biomarkers, collective biomarkers, collective status biomarkers, and collective prospective status biomarkers, can be used in sets or groups. For example, sets of biomarker loci, repetitive DNA sequences, repetitive DNA loci, biomarkers, status biomarkers prospective status biomarkers, collective biomarkers, collective status biomarkers, and collective prospective status biomarkers can include, for example, 2, 3, 4, 5, 6, 7, 8, 9, 10, 11, 12, 13, 14, 15, 16, 17, 18, 19, 20, 21, 22, 23, 24, 25, 26, 27, 28, 29, 30, 32, 34, 35, 36, 38, 40, 42, 44, 45, 46, 48, 50, 55, 60, 65, 70, 75, 80, 85, 90, 95, 100, 110, 120, 130, 140, 150, 160, 170, 180, 190, 200, 220, 240, 250, 260, 280, 300, 320, 340, 350, 360, 380, 400, 420, 440, 450, 460, 480, 500, 550, 600, 650, 700, 750, 800, 850, 900, 950, 1000, 1100, 1200, 1300, 1400, 1500, 1600, 1700, 1800, 1900, 2000, 2200, 2400, 2500, 2600, 2800, 3000, 3200, 3400, 3500, 3600, 3800, 4000, 4200, 4400, 4500, 4600, 4800, 5000, 5500, 6000, 6500, 700, 7500, 8000, 8500, 9000, 9500, 10,000, 11,000, 12,000, 13,000, 14,000, 15,000, 16,000, 17,000, 18,000, 19,000, 20,000, 22,000, 24,000, 25,000, 26,000, 28,000, 30,000, 32,000, 34,000, 35,000, 36,000, 38,000, 40,000, 42,000, 44,000, 45,000, 46,000, 48,000, 50,000, 55,000, 60,000, 65,000, 70,000, 75,000, 80,000, 85,000, 90,000, 95,000, 100,000, 110,000, 120,000, 130,000, 140,000, 150,000, 160,000, 170,000, 180,000, 190,000, 200,000, 210,000, 220,000, 230,000, 240,000, 250,000, 260,000, 270,000, 280,000, 290,000, 300,000, 320,000, 340,000, 350,000, 360,000, 380,000, 400,000 or more biomarker loci, repetitive DNA sequences, repetitive DNA loci, biomarkers, status biomarkers prospective status biomarkers, collective biomarkers, collective status biomarkers, or collective prospective status biomarkers, respectively. For collective biomarkers, the group of biomarkers making up the collective biomarker can include a number of individual biomarkers as described herein.

As another example, sets of biomarker loci, repetitive DNA sequences, repetitive DNA loci, biomarkers, status biomarkers prospective status biomarkers, collective biomarkers, collective status biomarkers, and collective prospective status biomarkers can include, for example, exactly or at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 32, 34, 35, 36, 38, 40, 42, 44, 45, 46, 48, 50, 55, 60, 65, 70, 75, 80, 85, 90, 95, 100, 110, 120, 130, 140, 150, 160, 170, 180, 190, 200, 220, 240, 250, 260, 280, 300, 320, 340, 350, 360, 380, 400, 420, 440, 450, 460, 480, 500, 550, 600, 650, 700, 750, 800, 850, 900, 950, 1000, 1100, 1200, 1300, 1400, 1500, 1600, 1700, 1800, 1900, 2000, 2200, 2400, 2500, 2600, 2800, 3000, 3200, 3400, 3500, 3600, 3800, 4000, 4200, 4400, 4500, 4600, 4800, 5000, 5500, 6000, 6500, 700, 7500, 8000, 8500, 9000, 9500, 10,000, 11,000, 12,000, 13,000, 14,000, 15,000, 16,000, 17,000, 18,000, 19,000, 20,000, 22,000, 24,000, 25,000, 26,000, 28,000, 30,000, 32,000, 34,000, 35,000, 36,000, 38,000, 40,000, 42,000, 44,000, 45,000, 46,000, 48,000, 50,000, 55,000, 60,000, 65,000, 70,000, 75,000, 80,000, 85,000, 90,000, 95,000, 100,000, 110,000, 120,000, 130,000, 140,000, 150,000, 160,000, 170,000, 180,000, 190,000, 200,000, 210,000, 220,000, 230,000, 240,000, 250,000, 260,000, 270,000, 280,000, 290,000, 300,000, 320,000, 340,000, 350,000, 360,000, 380,000, 400,000 biomarker loci, repetitive DNA sequences, repetitive DNA loci, biomarkers, status biomarkers prospective status biomarkers, collective biomarkers, collective status biomarkers, or collective prospective status biomarkers, respectively.

As another example, sets of biomarker loci, repetitive DNA sequences, repetitive DNA loci, biomarkers, status biomarkers prospective status biomarkers, collective biomarkers, collective status biomarkers, and collective prospective status biomarkers can include, for example, any range of from 2, 3, 4, 5, 6, 7, 8, 9, 10, 11, 12, 13, 14, 15, 16, 17, 18, 19, 20, 21, 22, 23, 24, 25, 26, 27, 28, 29, 30, 32, 34, 35, 36, 38, 40, 42, 44, 45, 46, 48, 50, 55, 60, 65, 70, 75, 80, 85, 90, 95, 100, 110, 120, 130, 140, 150, 160, 170, 180, 190, 200, 220, 240, 250, 260, 280, 300, 320, 340, 350, 360, 380, 400, 420, 440, 450, 460, 480, 500, 550, 600, 650, 700, 750, 800, 850, 900, 950, 1000, 1100, 1200, 1300, 1400, 1500, 1600, 1700, 1800, 1900, 2000, 2200, 2400, 2500, 2600, 2800, 3000, 3200, 3400, 3500, 3600, 3800, 4000, 4200, 4400, 4500, 4600, 4800, 5000, 5500, 6000, 6500, 700, 7500, 8000, 8500, 9000, 9500, 10,000, 11,000, 12,000, 13,000, 14,000, 15,000, 16,000, 17,000, 18,000, 19,000, 20,000, 22,000, 24,000, 25,000, 26,000, 28,000, 30,000, 32,000, 34,000, 35,000, 36,000, 38,000, 40,000, 42,000, 44,000, 45,000, 46,000, 48,000, 50,000, 55,000, 60,000, 65,000, 70,000, 75,000, 80,000, 85,000, 90,000, 95,000, 100,000, 110,000, 120,000, 130,000, 140,000, 150,000, 160,000, 170,000, 180,000, 190,000, 200,000, 210,000, 220,000, 230,000, 240,000, 250,000, 260,000, 270,000, 280,000, 290,000, 300,000, 320,000, 340,000, 350,000, 360,000, or 380,000 biomarker loci, repetitive DNA sequences, repetitive DNA loci, biomarkers, status biomarkers prospective status biomarkers, collective biomarkers, collective status biomarkers, or collective prospective status biomarkers, respectively, to 3, 4, 5, 6, 7, 8, 9, 10, 11, 12, 13, 14, 15, 16, 17, 18, 19, 20, 21, 22, 23, 24, 25, 26, 27, 28, 29, 30, 32, 34, 35, 36, 38, 40, 42, 44, 45, 46, 48, 50, 55, 60, 65, 70, 75, 80, 85, 90, 95, 100, 110, 120, 130, 140, 150, 160, 170, 180, 190, 200, 220, 240, 250, 260, 280, 300, 320, 340, 350, 360, 380, 400, 420, 440, 450, 460, 480, 500, 550, 600, 650, 700, 750, 800, 850, 900, 950, 1000, 1100, 1200, 1300, 1400, 1500, 1600, 1700, 1800, 1900, 2000, 2200, 2400, 2500, 2600, 2800, 3000, 3200, 3400, 3500, 3600, 3800, 4000, 4200, 4400, 4500, 4600, 4800, 5000, 5500, 6000, 6500, 700, 7500, 8000, 8500, 9000, 9500, 10,000, 11,000, 12,000, 13,000, 14,000, 15,000, 16,000, 17,000, 18,000, 19,000, 20,000, 22,000, 24,000, 25,000, 26,000, 28,000, 30,000, 32,000, 34,000, 35,000, 36,000, 38,000, 40,000, 42,000, 44,000, 45,000, 46,000, 48,000, 50,000, 55,000, 60,000, 65,000, 70,000, 75,000, 80,000, 85,000, 90,000, 95,000, 100,000, 110,000, 120,000, 130,000, 140,000, 150,000, 160,000, 170,000, 180,000, 190,000, 200,000, 210,000, 220,000, 230,000, 240,000, 250,000, 260,000, 270,000, 280,000, 290,000, 300,000, 320,000, 340,000, 350,000, 360,000, 380,000, or 400,000 biomarker loci, repetitive DNA sequences, repetitive DNA loci, biomarkers, status biomarkers prospective status biomarkers, collective biomarkers, collective status biomarkers, or collective prospective status biomarkers, respectively.

The methylation state of any number (such as the numbers and ranges described above) of, for example, biomarker loci, repetitive DNA sequences, repetitive DNA loci, biomarkers, status biomarkers prospective status biomarkers, collective biomarkers, collective status biomarkers, or collective prospective status biomarkers can be determined. In some forms, the methylation state of more than 100 biomarkers can be determined. In some forms, the methylation state of more than 1000 biomarkers can be determined. In some forms, the methylation state of more than 10,000 biomarkers can be determined. In some forms, the methylation state of more than 100,000 biomarkers can be determined. In some forms, the methylation state of more than 200,000 biomarkers can be determined. In some forms, the biomarker loci, repetitive DNA sequences, repetitive DNA loci, biomarkers, status biomarkers prospective status biomarkers, collective biomarkers, collective status biomarkers, and collective prospective status biomarkers can comprise a set of biomarker loci, repetitive DNA sequences, repetitive DNA loci, biomarkers, status biomarkers prospective status biomarkers, collective biomarkers, collective status biomarkers, or collective prospective status biomarkers, respectively. The set can comprise any number (such as the numbers and ranges described above) of, for example, biomarker loci, repetitive DNA sequences, repetitive DNA loci, biomarkers, status biomarkers prospective status biomarkers, collective biomarkers, collective status biomarkers, or collective prospective status biomarkers. In some forms, the set can comprise more than 100 status biomarkers. In some forms, the set can comprise more than 1000 status biomarkers. In some forms, the set can comprise more than 10,000 status biomarkers. In some forms, the set can comprise more than 100,000 status biomarkers. In some forms, the set can comprise more than 200,000 status biomarkers.

In some forms, a plurality of the biomarkers can independently belong to one or more status biomarker families, wherein each biomarker in each status biomarker family comprises one or more repetitive DNA sequences that belong to a single family of repetitive DNA sequences such as the repetitive DNA sequence families listed in, for example, Table 1, Table 12, or Table 13. In some forms, a plurality of biomarkers can independently belong to 2, 3, 4, 5, 6, 7, 8, 9, 10, 11, 12, 13, 14, 15, 16, 17, 18, 19, 20, 21, 22, 23, 24, 25, 26, 27, 28, 29, 30, 32, 34, 35, 36, 38, 40, 42, 44, 45, 46, 48, 50, 55, 60, 65, 70, 75, 80, 85, 90, 95, 100, 110, 120, 130, 140, 150, 160, 170, 180, 190, 200, 220, 240, 250, 260, 280, 300, 320, 340, 350, 360, 380, 400, 420, 440, 450, 460, 480, 500, 550, 600, 650, 700, 750, 800, 850, 900, 950, 1000, 1100, 1200 or more status biomarker families. In some forms, a plurality of biomarkers can independently belong to three or more status biomarker families. In some forms, a plurality of biomarkers can independently belong to four or more status biomarker families. In some forms, a plurality of biomarkers can independently belong to five or more status biomarker families. In some forms, a plurality of biomarkers can independently belong to ten or more status biomarker families. In some forms, a plurality of biomarkers can independently belong to twenty or more status biomarker families.

In some forms, 100 or more biomarkers can belong to one or more of the status biomarker families. In some forms, 100 or more biomarkers can belong to each of the status biomarker families. In some forms, 200 or more biomarkers can belong to one or more of the status biomarker families. In some forms, 200 or more biomarkers can belong to each of the status biomarker families. In some forms, 300 or more biomarkers can belong to one or more of the status biomarker families. In some forms, 300 or more biomarkers can belong to each of the status biomarker families. In some forms, 400 or more biomarkers can belong to one or more of the status biomarker families. In some forms, the 400 or more biomarkers can belong to each of the status biomarker families. In some forms, a plurality of, for example, biomarker loci, repetitive DNA sequences, repetitive DNA loci, biomarkers, status biomarkers prospective status biomarkers, collective biomarkers, collective status biomarkers, and collective prospective status biomarkers can independently belong to 2, 3, 4, 5, 6, 7, 8, 9, 10, 11, 12, 13, 14, 15, 16, 17, 18, 19, 20, 21, 22, 23, 24, 25, 26, 27, 28, 29, 30, 32, 34, 35, 36, 38, 40, 42, 44, 45, 46, 48, 50, 55, 60, 65, 70, 75, 80, 85, 90, 95, 100, 110, 120, 130, 140, 150, 160, 170, 180, 190, 200, 220, 240, 250, 260, 280, 300, 320, 340, 350, 360, 380, 400, 420, 440, 450, 460, 480, 500, 550, 600, 650, 700, 750, 800, 850, 900, 950, 1000, 1100, 1200 or more families of biomarker loci, repetitive DNA sequences, repetitive DNA loci, biomarkers, status biomarkers prospective status biomarkers, collective biomarkers, collective status biomarkers, or collective prospective status biomarkers, respectively.

In some forms, the status biomarkers can comprise a set of status biomarkers. In some forms, the members of the set of status biomarkers can be status biomarkers that indicate the status of one or more specific statuses. In some forms, the one or more specific statuses can comprise, for example, wellness, level of health, risk to wellness, risk to level of health, status of the genome, genomic instability, aging, risk of aging, cancer, risk of cancer, head and neck cancer, risk of head and neck cancer, breast cancer, risk of breast cancer, lung cancer, risk of lung cancer, prostate cancer, risk of prostate cancer, colon cancer, risk of colon cancer, esophageal cancer, risk of esophageal cancer, ovarian cancer, risk of ovarian cancer, liver cancer, risk of liver cancer, pancreatic cancer, risk of pancreatic cancer, skin cancer, risk of skin cancer, melanoma, risk of melanoma, lymphoma, risk of lymphoma, leukemia, risk of leukemia, cervical cancer, risk of cervical cancer, cervical dysplasia, risk of cervical dysplasia, cervical intraepithelial neoplasia, risk of cervical intraepithelial neoplasia, tumor burden, stress response, diabetes, risk of diabetes, heart disease, risk of heart disease, and/or response to treatment.

In some forms, the one or more specific statuses can comprise the presence of a disease or condition. In some forms, the one or more specific statuses can comprise, for example, a lack of wellness, low level of health, risk to wellness, risk to level of health, poor status of the genome, genomic instability, aging, risk of aging, cancer, risk of cancer, head and neck cancer, risk of head and neck cancer, breast cancer, risk of breast cancer, lung cancer, risk of lung cancer, prostate cancer, risk of prostate cancer, colon cancer, risk of colon cancer, esophageal cancer, risk of esophageal cancer, ovarian cancer, risk of ovarian cancer, liver cancer, risk of liver cancer, pancreatic cancer, risk of pancreatic cancer, skin cancer, risk of skin cancer, melanoma, risk of melanoma, lymphoma, risk of lymphoma, leukemia, risk of leukemia, cervical cancer, risk of cervical cancer, cervical dysplasia, risk of cervical dysplasia, cervical intraepithelial neoplasia, risk of cervical intraepithelial neoplasia, tumor burden, stress response, diabetes, risk of diabetes, heart disease, and/or risk of heart disease.

1. Lists of Status Biomarkers

Analysis of methylation levels in biological samples relevant to subject status (for example, normal, margin, tumor) of loci having CpG islands or CpG islets as described herein resulted in identification of various loci showing significant differences in methylation levels based on different status. Such loci are a useful form of status biomarker. Status biomarkers can be grouped in various ways. One useful way to group status biomarkers is into families of repetitive DNA sequences to which the status marker belongs. As used herein, a status biomarker belongs to a repetitive DNA sequence family (or category, or subcategory, or class) if the status biomarker comprises a repetitive DNA sequence belonging to that repetitive DNA sequence family (or category, or subcategory, or class). Loci analyzed according to the methods described herein can also be grouped in various ways. One useful way to group loci is into families of repetitive DNA sequences to which the locus belongs. As used herein, a locus belongs to a repetitive DNA sequence family (or category, or subcategory, or class) if the locus comprises a repetitive DNA sequence belonging to that repetitive DNA sequence family (or category, or subcategory, or class). Groups of status biomarkers and groups of loci can themselves be considered status biomarkers. For example, a group of status biomarkers belonging to the LTR54B family of repetitive DNA sequences can be a status biomarker. Such status biomarkers that comprise a group of components (such as a group of individual status biomarkers) can be referred to as a collective status biomarker. The collective status biomarker comprising status biomarkers belonging to the LTR54B family of repetitive DNA sequences can be referred to as a LTR54B family status biomarker. Collective status biomarkers are useful when determining a collective property of the individual status biomarkers in the group of status biomarkers, such as the average methylation of the individual loci that make up the status biomarkers in a group of status biomarkers. Status biomarkers are referred to herein by different terms such as variables, classifiers, and category classifiers.

Various lists of such status biomarker markers (both individual and collective) are presented herein. The lists below are lists of collective status biomarkers (groups of status biomarkers) determined to exhibit showing significant differences in methylation levels based on different status of the tissue (normal, margin, tumor).

The first two lists below arose from utilizing a list of 569 variables (collective status biomarkers) (listed in Table 18), each comprising the average methylation value of all members of a family of repetitive elements that were probed in the microarrays. Each case (a case being a biological sample; 62 total samples were probed) was associated with a corresponding list of 569 values generated by the microarray analysis. After all 62 cases were analyzed using a statistical tool for classification (Support Vector Machine (SVM) or, alternatively, Random Forrest) two different lists emerged that yield the best classification results (that is, best identify the status of the case based on the methylation level). The status classification is the same in both experiments: normal tissue sample, vs. tumor tissue sample, vs. nontumor adjacent tissue sample. Both status classification runs were supervised, in the sense that the assignment of each sample (normal, tumor, or margin) was made by a pathologist. The resulting lists are not the same, since different combinations of variable are capable of yielding a reasonably good classifier, and particularly because there are many more variables (569) than there are cases (62). The third list below is the union of the top 75 categories in the first two lists. The resulting list of 138 categories is referred to herein as the Top 138 categories (or status biomarkers or repetitive DNA sequence families).

i. List of Top 75 Classifier Categories Obtained Using SVM Analysis, by Rank

LTR54B, MER11B, U1, MER34B, LTR56, THE1B-int, HERV9, LTR14C, HERVFH21, LTR6B, LTR46, centr, MLT1D-int, MER67D, HERVK11, PABL_B, MSR1, AluYa5/8, LTR10B, HERVK22, GSAT, LTR10B1, LTR17, LTR51, MER11A, Other, L1PA12, ERVL-B4, HERVK14, LTR29, LTR6A, ALR/Alpha, LTR48B, MER105, MER67A, HUERS-P1, LTR7B, L1PB1, L1PA15-16, LTR28, MSTB-int, LTR45B, LTR7Y, HERVL18, LTR30, HERVK9, LTR45C, LTR47A, THE1C, LTR66, SST1, MER34B-int, LTR65, MER44D, MER57A-int, HUERS-P2, MER6A, MER50B, MER41E, 7SK, HERVP71A, L1PBa1, MER44C, GSATII, LTR1B, LTR7, MER91C, LTR22, Harlequin, MLT1F1, L1M3f, THE1B, HUERS-P3, MER92B, Charlie3

ii. List of Top 75 Classifier Categories Obtained Using Random Forest Analysis, by Rank

MER67D, MER6, ERVL, MER66C, HUERS-P3, MLT1G1, MER4D, MLT2D, THE1B, MLT1A1, MER11B, Charlie5, MLT2B3, MER50B, MER70A, Charlie3, MER50, LTR2, MLT1A, HERVL, LTR33A, MSTB-int, Cheshire, MSTA, MER51B, MLT2B2, MSTC, LTR9B, LTR14B, HUERS-P2, MSTB1, MSTD, LTR52, LTR8, LTR8A, MER92B, LTR22, MER51A, LTR36, LTR54B, PABL_A, MER4D1, AcHobo, LTR48, MLT1A0, LTR1B, MSTB, MER11D, LTR19A, MLT1E2, MER115, MER11A, MER34C, THE1C, MLT2B1, L1PA10, MER4A1, MLT1E, MLT2B4, LTR10B, L1MA7, HERVFH21, LTR5, THE1D, L1MA1, LTR9, MER63A, LTR5A, L1PB4, ERVL-B4, MLT1F, MLT2A2, LTR14, HERVK9, MER11C.

iii. List of Top 138 Classifier Categories, by Rank

LTR54B, MER67D, MER11B, MER6, ERVL, U1, MER34B, MER66C, HUERS-P3, LTR56, MLT1G1, THE1B-int, HERV9, MER4D, LTR14C, MLT2D, HERVFH21, THE1B, LTR6B, MLT1A1, LTR46, centr, Charlie5, MLT1D-int, MLT2B3, MER50B, HERVK11, MER70A, Charlie3, PABL_B, MER50, MSR1, AluYa5/8, LTR2, LTR10B, MLT1A, HERVK22, HERVL, GSAT, LTR33A, LTR10B1, MSTB-int, Cheshire, LTR17, LTR51, MSTA, MER11A, MER51B, MLT2B2, SVA, SVA_A, SVA_B, SVA_C, SVA_D, SVA_E, SVA_F, L1PA12, MSTC, ERVL-B4, LTR9B, HERVK14, LTR14B, HUERS-P2, LTR29, LTR6A, MSTB1, ALR/Alpha, MSTD, LTR48B, LTR52, LTR8, MER105, LTR8A, MER67A, HUERS-P1, MER92B, LTR22, LTR7B, L1PB1, MER51A, L1PA15-16, LTR36, LTR28, PABL_A, LTR45B, MER4D1, AcHobo, LTR7Y, HERVL18, LTR48, LTR30, MLT1A0, HERVK9, LTR1B, LTR45C, MSTB, LTR47A, MER11D, LTR19A, THE1C, LTR66, MLT1E2, MER115, SST1, MER34B-int, LTR65, MER34C, MER44D, MER57A-int, MLT2B1, L1PA10, MER4A1, MER6A, MLT1E, MER41E, MLT2B4, 7SK, HERVP71A, L1MA7, L1PBa1, LTR5, MER44C, GSATII, THE1D, L1MA1, LTR7, LTR9, MER63A, MER91C, LTR5A, Harlequin, L1PB4, MLT1F1, L1M3f, MLT1F, MLT2A2, LTR14, MER11C.

569 repetitive DNA sequence families were identified from among the loci identified as CpG island- or CpG islet-containing loci as described in Example 2. Table 18 is a list of these repetitive DNA sequence families. Among the 569 repetitive element families comprising the full set of repetitive DNA sequence status biomarkers, a subset of 138 was identified that are most effective as classifiers. This subset was generated by merging the top 75 categories identified by a Random Forest analysis with another 75 categories that were the best performers using a Support Vector Machine classifier. This produced the list of Top 138 status biomarkers (Table 1). A Random Forest classification analysis was performed utilizing the set of Top 138 status biomarkers, and a second one utilizing the remainder of the 569 (a subset of 431). The list of this subset of 431 status biomarkers can be derived by eliminating the Top 138 status biomarkers in Table 1 from the list of 569 status biomarkers in Table 18. Random Forest analysis using the top 138 status biomarkers gave a classification error of 8.1%. The Receiver Operator Characteristic curves for this analysis gave an AUC of 1 for margin versus normal and an AUC of 0.91 for tumor versus margin. The second Random Forest analysis was performed using the remaining 431 status biomarkers. The classification error in this analysis was 19.0%. Thus, the Top 138 status biomarkers are significantly better for assessing the status of the samples and subjects than the remaining status biomarkers. In a separate experiment using SVM analysis, the superior performance of the top 138 status biomarkers compared to the remaining 431 variables was confirmed. These results provide an objective metric for claiming superior utility of the top 138 biomarkers for assessing status of subjects.

The utility of the Status Biomarkers for distinguishing dysplasia from cancer was optimized by performing a classification analysis that does not include the data from the normal samples, and which can be called a nontumor margin vs. tumor classification. Taking the 569 repetitive element categories as variables (Table 18), classification of margin vs. tumor using Random Forest was performed, and the best 75 variables were saved. Then, again taking the 569 repetitive element categories as variables, classification of margin vs. tumor using the Support vector machine was performed, and the best 75 variables were saved. The union of the best 75 RF variables and the best 75 SVM variables was then calculated, and this yielded 137 variables, which are called the Top performing variables for margin vs. tumor classification (Table 12).

The Top 137 variables were used to perform an RF classification, which yielded a classification error of 9.6%. Using the remaining 432 variables yielded a classification error of 17%, confirming the superior performance of the Top 137 variables.

TABLE 12
Top 137 performing variables for tumor versus margin in rank order.

| | |
|---|---|
| 1. | MER50B |
| 2. | MER94 |
| 3. | LTR19A |
| 4. | MLT2B3 |
| 5. | AcHobo |
| 6. | MER67A |
| 7. | HERV16 |
| 8. | LTR14C |
| 9. | L1MCb |
| 10. | LTR36 |
| 11. | HERVL40 |
| 12. | L1MC3 |
| 13. | HERVL |
| 14. | LTR56 |
| 15. | MER57A-int |
| 16. | LTR23 |
| 17. | MLT1E2 |
| 18. | ALR/Alpha |
| 19. | MER57A |
| 20. | L1MA9 |
| 21. | LTR27 |
| 22. | LTR51 |
| 23. | L1PA13 |
| 24. | MER34 |
| 25. | HERV9 |
| 26. | MER11A |
| 27. | MER102b |
| 28. | THE1B-int |
| 29. | L1P2 |
| 30. | MER31B |
| 31. | L1MD1 |
| 32. | MER91A |
| 33. | HSMAR1 |
| 34. | MER34B |
| 35. | FLAM_A |
| 36. | LTR1D |
| 37. | L1MC1 |
| 38. | MLT1B |
| 39. | Charlie3 |
| 40. | LTR54B |
| 41. | HERVL18 |
| 42. | L1MB4 |
| 43. | LTR26 |
| 44. | MER90a |
| 45. | U1 |
| 46. | MSTC |
| 47. | MER106B |
| 48. | Tigger1 |
| 49. | LTR9B |
| 50. | MER11D |
| 51. | L1MA5A |
| 52. | MER93B |
| 53. | L1MB1 |
| 54. | LTR10B |
| 55. | MIR3 |
| 56. | PRIMA4-int |
| 57. | HERVK11 |
| 58. | THE1B |
| 59. | MER63D |
| 60. | L1P3 |
| 61. | MER119 |
| 62. | L1MC2 |
| 63. | LTR18B |
| 64. | LTR9 |
| 65. | MER6 |
| 66. | MLT1L |
| 67. | MLT2D |
| 68. | MER44B |
| 69. | MER50 |
| 70. | tRNA |
| 71. | MLT1E |
| 72. | HERV35I |
| 73. | L1PA6 |
| 74. | MLT1A0 |
| 75. | L1MD |
| 76. | MER69B |
| 77. | L1PB2 |
| 78. | MER21A |
| 79. | Tip100 |
| 80. | LTR26B |
| 81. | L1ME2 |
| 82. | L1PA15 |
| 83. | ERVL |
| 84. | MER81 |
| 85. | L1P5 |
| 86. | LTR16B |
| 87. | ERVK |
| 88. | LTR6B |
| 89. | HERVL-A1 |
| 90. | LTR24C |
| 91. | L1MA10 |
| 92. | LTR16A |
| 93. | L1M4b |
| 94. | MER46B |
| 95. | L1P1 |
| 96. | MSR1 |
| 97. | HUERS-P2 |
| 98. | L1P4 |
| 99. | LTR12D |
| 100. | MER34B-int |
| 101. | LTR12_{—} |
| 102. | L1M3d |
| 103. | L1PA3 |
| 104. | LTR2B |
| 105. | MER65D |
| 106. | MLT1E1 |
| 107. | MLT1J2 |
| 108. | AluSg/x |
| 109. | L3b |
| 110. | LTR5_Hs |
| 111. | AluYa8 |
| 112. | MER110A |
| 113. | AluYa5/8 |
| 114. | L1PA2 |
| 115. | L1MA5 |
| 116. | MER91C |
| 117. | LTR67 |
| 118. | MER21-int |
| 119. | HAL1 |
| 120. | MER63A |
| 121. | LTR10B1 |
| 122. | MIRb |
| 123. | L1PBa1 |
| 124. | SST1 |
| 125. | Charlie10 |
| 126. | Tigger7 |
| 127. | AluYa5 |
| 128. | MSTA-int |
| 129. | L1PA7 |
| 130. | LTR22C |
| 131. | LTR54 |
| 132. | MER50-int |
| 133. | HERVK14 |
| 134. | HUERS-P3 |
| 135. | MER77 |
| 136. | MLT1A |
| 137. | THE1C |

The overlap between the Top 138 Variables (Table 1) that are the best classifiers for normal vs. margin vs. tumor and the Top 137 categories (Table 12) that are the best classifiers for margin vs. tumor was calculated. The comparison shows that only 48 variables are common to both lists and thus are good classifiers for both tumor-margin and tumor-margin-normal comparison experiments. The 48 common variables are listed below in Table 13.

TABLE 13
List of 48 variables common to Top 137 and Top 138.

ALR/Alpha
AluYa5/8
AcHobo
Charlie3
ERVL
HERV9
HERVK11
HERVK14
HERVL
HERVL18
HUERS-P2
HUERS-P3
L1PBa1
LTR10B
LTR10B1
LTR14C
LTR19A
LTR36
LTR51
LTR54B
LTR56
LTR6B
LTR9
LTR9B
MER11A
MER11D
MER34B-int
MER34B
MER50
MER50B
MER57A-int
MER6
MER63A
MER67A
MER91C
MLT1A
MLT1A0
MLT1E
MLT1E2
MLT2B3
MSR1
MSTC
SST1
THE1B-int
THE1B
THE1C
U1

The 137 categories from Table 12 minus the 48 common variables from Table 13 result in a list of 89 different variables that are good classifiers among tumor and margin comparison experiments but not for tumor-margin-normal comparison experiments. The list of 89 different variables is as follows: AluSg/x, AluYa5, AluYa8, tRNA, Charlie 10, ERVK, FLAM_A, HAL1, HERV16, HERV351, HERVL-A1, HERVL40, HSMAR1, L1M3d, L1M4b, L1MA10, L1MA5, L1MA5A, L1MA9, L1MB1, L1MB4, L1MC1, L1MC2, L1MC3, L1MCb, L1MD, L1MD1, L1ME2, L1P1, L1P2, L1P3, L1P4, L1P5, L1PA13, L1PA15, L1PA2, L1PA3, L1PA6, L1PA7, L1PB2, L3b, LTR12, LTR12D, LTR16A, LTR16B, LTR18B, LTR1D, LTR22C, LTR23, LTR24C, LTR26, LTR26B, LTR27, LTR2B, LTR5_Hs, LTR54, LTR67, MER102b, MER106B, MER110A, MER119, MER21-int, MER21A, MER31B, MER34, MER44B, MER46B, MER50-int, MER57A, MER63D, MER65D, MER69B, MER77, MER81, MER90a, MER91A, MER93B, MER94, MIR3, MIRb, MLT1B, MLT1E1, MLT1J2, MLT1L, MSTA-int, PRIMA4-int, Tigger1, Tigger7, Tip100.

The 138 categories from Table 1 minus the 48 common variables in Table 13 result in a list of 90 different variables that are good classifiers among tumor-margin-normal comparison experiments but not for tumor-margin comparisons. The list of 90 different variables is as follows: 7SK, centr, SVA, Charlie5, Cheshire, ERVL-B4, GSAT, GSATII, Harlequin, HERVFH21, HERVK22, HERVK9, HERVP71A, HUERS-P1, L1M3f, L1MA1, L1MA7, L1PA10, L1PA12, L1PA15-16, L1PB1, L1PB4, LTR14, LTR14B, LTR17, LTR1B, LTR2, LTR22, LTR28, LTR29, LTR30, LTR33A, LTR45B, LTR45C, LTR46, LTR47A, LTR48, LTR48B, LTR5, LTR52, LTR5A, LTR65, LTR66, LTR6A, LTR7, LTR7B, LTR7Y, LTR8, LTR8A, MER105, MER115, MER11B, MER11C, MER34C, MER41E, MER44C, MER44D, MER4A1, MER4D, MER4D1, MER51A, MER51B, MER66C, MER67D, MER6A, MER70A, MER92B, MLT1A1, MLT1D-int, MLT1F, MLT1F1, MLT1G1, MLT2A2, MLT2B1, MLT2B2, MLT2B4, MSTA, MSTB-int, MSTB, MSTB1, MSTD, PABL_A, PABL_B, SVA_A, SVA_B, SVA_C, SVA_D, SVA_E, SVA_F, THE1D.

Table 14 reports the repetitive element families present in a 600-base window centered on each microarray probe. This is an example of neighbor repeat analysis. The presence of repetitive DNA sequences belonging to different families of repetitive DNA sequences in the same, for example, status biomarker or repetitive DNA sequence locus can facilitate some of the forms of the disclosed methods. For example, the different repetitive DNA sequences can be used to define a PCR amplicon by, for example, using primers specific for two of the different repetitive DNA sequences.

TABLE 14
List of neighboring repeats, and their frequencies, that occur in the
neighborhood of the probes in the Top 138 categories.

| Probe | Frequency of other repeats in 2 × 300 window around the |
|---|---|
| Category | probe |
| LTR54B | AluY - [41.51%], AluSx - [16.98%], AluSp - [11.32%], AluJb - [9.43%], |
| | AluSq - [7.55%], MER57A-int - [5.66%], MIR - |
| | [5.66%], LTR54 - [5.66%] |
| MER67D | AluY - [24.49%], AluSp - [10.20%], AluSx - [10.20%], |
| | MER67C - [8.16%], AluSc - [6.12%], L1MC3 - [6.12%] |
| MER11B | AluY - [9.76%], L2 - [7.32%] |
| MER6 | AluY - [28.17%], AluSx - [14.49%], AluSg - [6.67%], AluSq - [6.07%], |
| | L2 - [5.56%], AluSc - [5.21%] |
| ERVL | AluY - [30.40%], AluSx - [13.05%], LTR33 - [7.37%], LTR16C - |
| | [7.20%], AluSg - [6.02%], L2 - [5.43%], AluSc - [5.03%] |
| U1 | AluSx - [27.87%], AluY - [16.39%], AluSg - [9.84%], AluSp - |
| | [9.84%] |
| MER34B | AluY - [43.97%], AluSx - [22.41%], AluSq - [7.76%], AluSp - |
| | [7.76%], MIRb - [6.90%], AluSg - [6.03%] |
| MER66C | AluY - [29.63%], AluSx - [20.37%], AluSq - [9.26%], AluSc - |
| | [7.41%], AluSp - [5.56%] |
| HUERS-P3 | AluY - [43.62%], LTR9 - [17.02%], LTR9B - [14.89%], AluSg - |
| | [8.51%], AluSp - [6.38%] |
| LTR56 | AluY - [32.53%], AluSx - [22.89%], AluSq - [10.84%], AluSg - |
| | [7.23%], AluSp - [7.23%] |
| MLT1G1 | AluY - [29.55%], AluSx - [14.17%], L2 - [8.50%], AluSg - |
| | [6.48%], AluSc - [5.67%] |
| THE1B-int | THE1B - [43.48%], AluY - [28.26%], AluYa5 - [10.87%], |
| | LTR12C - [6.52%] |
| HERV9 | LTR12C - [22.97%], LTR12 - [10.14%], LTR12_- [8.78%], |
| | LTR12D - [8.11%], AluY - [6.76%] |
| MER4D | AluY - [31.58%], AluSx - [15.79%], AluSq - [7.29%], AluSg - |
| | [6.48%], MER49 - [5.26%], AluSc - [5.26%] |
| LTR14C | AluY - [20.00%], HERVK14C - [16.67%], AluSx - [10.00%], |
| | AluJo - [6.67%], LTR12C - [6.67%], Tigger1 - [6.67%] |
| MLT2D | AluY - [32.18%], AluSx - [9.77%], L2 - [6.90%], AluSp - |
| | [6.32%], AluSg - [5.75%], AluSc - [5.17%], AluSq - [5.17%] |
| HERVFH21 | AluY - [16.67%], LTR21A - [10.00%], LTR21B - [8.33%] |
| THE1B | AluY - [28.51%], AluSx - [8.34%], L2 - [6.95%] |
| LTR6B | HERVS71 - [29.55%], AluSx - [6.82%], MIR - [6.82%] |
| MLT1A1 | AluY - [32.71%], AluSx - [12.77%], AluSc - [7.48%], AluSp - |
| | [7.17%], AluSg - [6.54%], L2 - [5.61%] |
| LTR46 | AluSx - [17.95%], AluY - [15.38%], AluSg - [7.69%], L2 - |
| | [7.69%], AluJb - [5.13%] |
| centr | SST1 - [29.21%], GSATII - [12.70%], ALR/Alpha - [9.21%] |
| Charlie5 | AluY - [29.12%], AluSx - [21.07%], L2 - [8.43%], AluSg - |
| | [7.28%], AluSq - [6.90%], AluJo - [5.75%], AluSp - |
| | [5.75%], AluJb - [5.36%] |
| MLT1D-int | MLT1D - [36.84%], AluY - [31.58%], AluSx - [13.16%], AluSc - |
| | [10.53%], AluSp - [7.89%], AluSg - [5.26%], AluJb - |
| | [5.26%], AluYa5 - [5.26%] |
| MLT2B3 | AluY - [37.88%], AluSx - [8.33%], AluSg - [7.58%], MIRb - |
| | [7.58%], L2 - [6.82%] |
| MER50B | AluY - [30.39%], AluSx - [15.69%], AluSc - [8.82%] |
| HERVK11 | MER11C - [12.82%], AluY - [10.26%], MLT1D - [5.13%], |
| | MER11D - [5.13%], MER11A - [5.13%] |
| MER70A | AluY - [28.21%], AluSx - [17.95%], L2 - [7.69%], MLT1A - |
| | [5.13%], MIRb - [5.13%], Charlie4a - [5.13%], MSTB - |
| | [5.13%], MER70B - [5.13%] |
| Charlie3 | AluSx - [17.54%], AluY - [17.54%], AluSg - [8.77%], MIR - |
| | [8.77%], MIRb - [5.26%] |
| PABL_B | AluY - [19.44%], AluSx - [13.89%], AluSg - [5.56%], AluSq - |
| | [5.56%], AluSp - [5.56%], L2 - [5.56%] |
| MER50 | AluY - [25.74%], AluSx - [12.75%], AluSg - [7.11%], AluSc - |
| | [6.13%] |
| MSR1 | AluSx - [7.63%] |
| LTR2 | AluY - [18.33%], AluSx - [11.26%], AluSg - [5.95%], |
| | Harlequin - [5.30%] |
| LTR10B | AluSg - [18.60%], AluY - [18.60%], AluS - [11.63%], AluSp - |
| | [9.30%], AluSc - [6.98%] |
| MLT1A | AluY - [32.36%], AluSx - [12.68%], L2 - [6.23%], AluSg - |
| | [5.89%], AluSc - [5.27%], AluSp - [5.16%], AluSq - [5.10%] |
| HERVK22 | LTR22C - [18.07%], LTR22A - [8.43%], AluY - [8.43%], |
| | LTR22B - [6.02%] |
| HERVL | AluY - [23.11%], AluSq - [6.72%], AluSx - [6.72%], AluSg - |
| | [6.30%], LTR18A - [5.04%] |
| LTR33A | AluY - [25.86%], L2 - [10.92%], AluSx - [10.92%] |
| LTR10B1 | AluSg - [22.86%], AluY - [17.14%], AluS - [14.29%], AluSp - |
| | [11.43%], AluSc - [5.71%], MER93b - [5.71%] |
| MSTB-int | AluY - [38.24%], MSTB - [35.29%], AluSc - [11.76%], AluSq - |
| | [8.82%], AluSx - [8.82%], AluYa5 - [8.82%] |
| Cheshire | AluY - [33.33%], AluSx - [31.82%], AluSg - [15.15%], AluSp - |
| | [6.06%], AluJb - [6.06%] |
| LTR17 | HERV17 - [12.24%] |
| LTR51 | AluY - [44.12%], AluSx - [23.53%], AluSg - [5.88%], AluSc - |
| | [5.88%], AluSp - [5.88%], AluJb - [5.88%], L2 - [5.88%] |
| MSTA | AluY - [36.17%], AluSx - [10.73%], AluSg - [5.53%], L2 - |
| | [5.53%] |
| MER11A | AluY - [13.95%], AluSg - [6.98%] |
| MER51B | AluY - [25.56%], AluSx - [23.33%], AluSg - [8.89%], AluSq - |
| | [6.67%], AluSp - [6.67%], AluSc - [5.56%] |
| MLT2B2 | AluY - [30.77%], AluSx - [12.09%], AluSc - [7.69%], L2 - |
| | [7.69%] |
| SVA | AluSx - [7.14%] |
| L1PA12 | AluY - [28.68%], AluSp - [8.82%], AluSx - [8.09%], AluSg - |
| | [5.15%] |
| MSTC | AluY - [41.23%], AluSx - [15.64%], AluSg - [7.58%], AluSq - |
| | [7.58%], AluSc - [5.69%], L2 - [5.69%] |
| ERVL-B4 | AluY - [32.33%], AluSg - [8.27%], AluSx - [8.27%], AluSq - |
| | [7.52%] |
| LTR9B | AluY - [23.70%], AluSx - [8.15%], AluSp - [6.67%], HUERS- |
| | P3b - [5.93%] |
| HERVK14 | LTR14B - [26.56%], AluY - [18.75%], LTR14A - [10.94%], |
| | LTR14C - [7.81%], MER41C - [6.25%] |
| LTR14B | HERVK14 - [28.81%], AluY - [10.17%], AluSx - [6.78%], MIRb - |
| | [5.08%] |
| HUERS-P2 | AluY - [31.25%], LTR1 - [21.88%], LTR1C - [9.38%], AluSg - |
| | [6.25%], LTR12C - [6.25%], AluSc - [6.25%] |
| LTR29 | AluY - [38.89%], AluSx - [20.37%], AluSg - [11.11%], AluSp - |
| | [11.11%], LTR49 - [9.26%], AluSc - [5.56%], AluJo - |
| | [5.56%], MER39 - [5.56%], FLAM_C - [5.56%] |
| LTR6A | L2 - [10.96%], HERVS71 - [9.59%], AluY - [6.85%], AluSq - |
| | [5.48%], AluJb - [5.48%], AluSx - [5.48%] |
| MSTB1 | AluY - [37.05%], AluSx - [12.05%], L2 - [7.14%], AluSg - |
| | [6.70%], AluSp - [6.70%], AluSc - [6.25%], AluSq - [5.36%] |
| MSTD | AluY - [36.16%], AluSx - [14.25%], AluSg - [10.14%], L2 - |
| | [7.67%], AluSc - [6.03%], AluSq - [5.75%] |
| LTR48B | AluY - [33.93%], AluSx - [14.29%], MER39 - [12.50%], AluSp - |
| | [8.93%], AluSg - [7.14%], AluSq - [7.14%], AluJb - |
| | [5.36%], AluYa5 - [5.36%], MER34 - [5.36%] |
| LTR52 | AluY - [42.03%], AluSx - [10.14%], AluSg - [7.25%], AluSc - |
| | [7.25%], AluSp - [7.25%], AluSq - [5.80%] |
| LTR8 | AluY - [28.19%], AluSx - [17.49%], AluSg - [7.20%], AluSq - |
| | [6.58%], AluSp - [5.97%] |
| MER105 | AluY - [16.22%], AluSg - [13.51%], L2 - [13.51%], AluSx - |
| | [10.81%], AluSq - [8.11%], MER5A - [8.11%], AluSp - |
| | [5.41%], AluSg/x - [5.41%], MIR - [5.41%], L1PA3 - [5.41%] |
| LTR8A | AluY - [27.27%], AluSx - [18.83%], AluSq - [6.49%], AluSg - |
| | [5.84%] |
| MER67A | AluY - [20.51%], AluSc - [12.82%], AluSq - [12.82%], AluSx - |
| | [10.26%], MER67B - [10.26%], AluJo - [7.69%], AluSg - |
| | [5.13%], LTR12C - [5.13%], L2 - [5.13%] |
| HUERS-P1 | AluY - [33.33%], AluSx - [19.05%], AluSp - [16.67%], LTR8 - |
| | [16.67%], LTR8A - [7.14%], AluSc - [7.14%] |
| MER92B | AluY - [31.94%], AluSx - [18.06%], AluSc - [9.72%], AluSp - |
| | [6.94%], AluSg - [5.56%], AluSq - [5.56%] |
| LTR22 | HERVK22 - [32.95%], AluY - [17.05%], L2 - [5.68%] |
| LTR7B | HERVH - [28.47%], AluY - [13.87%], AluSx - [7.30%], L2 - |
| | [5.84%] |
| L1PB1 | AluY - [27.26%], AluSx - [8.12%], AluSg - [5.82%] |
| MER51A | AluY - [28.85%], AluSx - [12.50%], AluSg - [8.65%], AluSq - |
| | [6.73%], L2 - [6.73%], AluSp - [5.77%] |
| L1PA15-16 | AluY - [45.31%], AluSx - [10.94%] |
| LTR36 | AluY - [42.00%], AluSx - [18.00%], AluSq - [10.00%] |
| LTR28 | AluY - [23.33%], AluSx - [6.67%], L1MA9 - [6.67%], LTR27B - |
| | [6.67%] |
| PABL_A | AluY - [25.81%], AluSx - [12.90%], AluSg - [8.06%], AluSc - |
| | [8.06%], AluSq - [8.06%] |
| LTR45B | AluSx - [20.59%], AluY - [14.71%], AluSg - [11.76%], |
| | LTR72B - [8.82%], AluSq - [5.88%], AluSp - [5.88%], MER5B - |
| | [5.88%], L2 - [5.88%], MIR - [5.88%] |
| MER4D1 | AluY - [35.45%], AluSx - [12.73%], AluSq - [10.00%], AluSg - |
| | [6.36%] |
| AcHobo | AluY - [27.88%], MER63 - [21.36%], MER94 - [18.56%], MER81 - |
| | [17.35%], AluSx - [13.84%], MER63A - [12.14%], BLACKJACK - |
| | [10.03%], L2 - [8.63%], MER63D - [6.72%], AluSq - |
| | [6.22%], AluSg - [5.82%], MER63C - [5.52%] |
| LTR7Y | HERVH - [30.39%], L2 - [7.84%], AluSx - [5.88%], MIRb - |
| | [5.88%] |
| HERVL18 | LTR18A - [28.57%], AluY - [11.90%] |
| LTR48 | AluY - [33.86%], AluSg - [14.17%], AluSx - [14.17%], MER34 - |
| | [5.51%], MER39 - [5.51%], AluSc - [5.51%], AluSp - |
| | [5.51%] |
| LTR30 | HERV30 - [20.93%], AluY - [9.30%], AluSx - [6.98%] |
| MLT1A0 | AluY - [31.74%], AluSx - [12.66%], L2 - [6.42%], AluSg - |
| | [5.66%], AluSq - [5.27%] |
| HERVK9 | MER9 - [46.75%], AluY - [7.79%], MER9B - [6.49%] |
| LTR1B | AluY - [13.64%], AluSx - [6.36%] |
| LTR45C | AluY - [35.29%], AluSx - [23.53%], AluSq - [8.82%], AluJo - |
| | [8.82%], AluSc - [5.88%], AluJb - [5.88%] |
| MSTB | AluY - [35.01%], AluSx - [15.19%], L2 - [7.08%], AluSc - |
| | [5.79%], AluSg - [5.79%], AluSp - [5.02%] |
| LTR47A | AluY - [32.20%], AluSx - [13.56%], AluSc - [8.47%], AluSp - |
| | [8.47%], AluSq - [5.08%] |
| MER11D | AluY - [16.22%], AluSx - [10.81%], MER58A - [5.41%], |
| | HERVK11D - [5.41%] |
| LTR19A | AluY - [29.73%], AluSq - [18.92%], AluSx - [13.51%], AluSg - |
| | [5.41%], SVA_D - [5.41%], L2 - [5.41%], HERVFH19 - |
| | [5.41%], L1M5 - [5.41%] |
| THE1C | AluY - [26.65%], AluSx - [12.89%], L2 - [6.59%], AluSg - |
| | [5.44%] |
| LTR66 | HERVL66 - [22.73%], AluY - [20.45%], AluSc - [9.09%], |
| | AluSg - [6.82%] |
| MLT1E2 | AluY - [34.31%], AluSx - [11.30%], AluSc - [8.37%], L2 - |
| | [6.69%] |
| MER115 | AluY - [26.24%], AluSx - [11.35%], AluSp - [5.67%] |
| MER34B-int | AluY - [54.84%], AluSx - [22.58%], AluSp - [12.90%], AluSc - |
| | [6.45%], AluSq - [6.45%], AluJb - [6.45%], MER34 - |
| | [6.45%] |
| LTR65 | AluY - [23.53%], AluSx - [14.71%], AluSq - [11.76%], AluSg - |
| | [8.82%], L2 - [8.82%], MIR - [8.82%], AluJo - [5.88%] |
| MER34C | AluY - [29.21%], AluSx - [11.24%], AluSg - [8.99%], |
| | MER57A-int - [8.99%], AluSc - [7.87%], AluSp - [5.62%] |
| MER44D | AluY - [30.00%], AluSg - [10.00%], MIRb - [10.00%], AluSq - |
| | [6.67%], AluSp - [6.67%], LTR33 - [6.67%] |
| MER57A-int | AluY - [40.94%], AluSx - [14.17%], AluSc - [7.09%], AluSq - |
| | [7.09%], MER34C - [5.51%] |
| MLT2B1 | AluY - [29.59%], AluSx - [14.20%], AluSc - [6.51%], AluSg - |
| | [5.33%] |
| L1PA10 | AluY - [29.82%], AluSx - [9.36%], AluSc - [5.56%] |
| MER4A1 | AluY - [26.85%], AluSx - [10.89%], AluSg - [8.17%], AluSp - |
| | [5.84%] |
| MER6A | AluY - [24.71%], AluSx - [14.12%], L2 - [9.41%], AluSg - |
| | [8.24%], AluJb - [7.06%] |
| MLT1E | AluY - [35.75%], AluSx - [11.71%], AluSc - [6.47%], L2 - |
| | [5.08%] |
| MER41E | AluY - [19.44%], AluSx - [16.67%], AluSg - [13.89%], AluSc - |
| | [5.56%], AluSq - [5.56%], AluSp - [5.56%], MIR - |
| | [5.56%], L1M4 - [5.56%], L1M5 - [5.56%] |
| MLT2B4 | AluY - [27.39%], AluSx - [17.83%], D20S16 - [8.28%], AluSc - |
| | [5.73%], AluSq - [5.73%], L2 - [5.10%] |
| 7SK | AluY - [20.69%], AluSx - [13.79%], L2 - [6.90%] |
| HERVP71A | AluY - [34.21%], LTR71A - [21.05%], LTR71B - [13.16%], |
| | LTR18B - [10.53%], AluSx - [5.26%] |
| L1MA7 | AluY - [34.46%], AluSx - [12.53%], AluSg - [8.36%] |
| L1PBa1 | AluY - [16.67%], LTR12C - [10.42%], AluSc - [6.25%] |
| LTR5 | AluY - [24.79%], AluSx - [14.22%], AluSg - [5.29%], AluSp - |
| | [5.17%] |
| MER44C | AluY - [36.84%], AluSx - [26.32%], AluSg - [5.26%], AluSc - |
| | [5.26%], AluSq - [5.26%] |
| GSATII | GSATX - [5.48%] |
| THE1D | AluY - [31.31%], AluSx - [11.15%], AluSg - [6.85%], L2 - |
| | [6.26%], AluSq - [5.48%] |
| L1MA1 | AluY - [39.07%], AluSx - [12.55%], L1ME1 - [8.91%], AluSg - |
| | [7.69%] |
| LTR7 | HERVH - [17.75%], AluY - [11.23%], L2 - [5.45%] |
| LTR9 | AluY - [19.44%], AluSx - [10.77%], HUERS-P3 - [5.15%] |
| MER63A | AluY - [33.86%], L2 - [10.24%], AluSx - [9.45%], AluSq - |
| | [6.30%] |
| MER91C | AluY - [25.00%], L2 - [9.09%], AluSx - [6.82%], MIRb - |
| | [6.82%] |
| LTR5A | HERVK - [14.71%], MIRb - [11.76%], AluSx - [5.88%], L2 - |
| | [5.88%], MER2 - [5.88%] |
| Harlequin | LTR2 - [28.68%], LTR2B - [13.18%], AluY - [11.63%] |
| L1PB4 | AluY - [35.87%], AluSx - [14.59%], AluSc - [5.78%], AluSq - |
| | [5.78%], AluSg - [5.47%] |
| MLT1F1 | AluY - [34.31%], AluSx - [21.57%], AluSc - [7.35%], AluSg - |
| | [6.37%] |
| L1M3f | AluY - [42.86%], AluSx - [9.09%], AluSg - [5.19%], AluSq - |
| | [5.19%], AluJo - [5.19%] |
| MLT1F | AluY - [31.82%], AluSx - [17.63%], AluSg - [7.58%], L2 - |
| | [7.02%], AluSc - [5.79%], AluSp - [5.23%] |
| MLT2A2 | AluY - [24.26%], AluSx - [8.09%], AluSg - [6.62%], L2 - |
| | [5.15%], HERVL-A2 - [5.15%] |
| LTR14 | HERVK14 - [18.90%], AluY - [12.60%], AluSx - [7.87%] |
| MER11C | AluY - [6.52%], L2 - [5.07%] |

A very interesting feature of this analysis is the presence of LTR2 and LTR2B repetitive elements in the vicinity of Harlequin repeats, which are a special type of LTR repeat. A report in the journal “Oncogene” described an unusual set of human genes known as HOST genes, which contain sequences comprising a mixture of Harlequin repetitive elements joined to LTR2 repetitive elements (Rangel et al., 2003). HOST genes are overexpressed in ovarian cancer (Rangel et al., 2003). The presence of the Harlequin class of repeats in the list of the best classifier probes found by the Support Vector Machine analysis indicates the existence of a large number of genomic loci with a structure similar to that of the ovarian cancer HOST genes. These unusual loci suffer major changes in DNA methylation status in cancers of the head and neck, as revealed by analysis herein.

Table 16 is a list of 126 repetitive element families that occur as neighbors in a window of 2×300 bases near the Top 138 classifier probes.

TABLE 16
List of 126 repetitive element families that occur as neighbors in a
window of 2 × 300 bases near the Top 138 classifier probes.

| | how often it co- | | |
|---|---|---|---|
| | localizes with other | | |
| | repeats, near 35235 | | # of members of |
| | probes, members of | | windows of 2 × 300 |
| Repeat | the 138 categories | percentage | bases considered |
| AluY | 9288 | 26.36% | 35235 |
| AluSx | 4062 | 11.53% | 35235 |
| MLT1A0 | 1932 | 5.48% | 35235 |
| AluSg | 1668 | 4.73% | 35235 |
| L2 | 1349 | 3.83% | 35235 |
| AluSc | 978 | 2.78% | 35235 |
| MSTA | 838 | 2.38% | 35235 |
| AluSq | 686 | 1.95% | 35235 |
| THE1B | 686 | 1.95% | 35235 |
| MLT1A1 | 584 | 1.66% | 35235 |
| L1PB1 | 570 | 1.62% | 35235 |
| SVA_D | 527 | 1.50% | 35235 |
| LTR7 | 506 | 1.44% | 35235 |
| AluSp | 479 | 1.36% | 35235 |
| THE1D | 470 | 1.33% | 35235 |
| MSTB | 464 | 1.32% | 35235 |
| LTR33 | 454 | 1.29% | 35235 |
| LTR16C | 441 | 1.25% | 35235 |
| MLT1E2 | 438 | 1.24% | 35235 |
| ERVL-E | 431 | 1.22% | 35235 |
| MSTB1 | 430 | 1.22% | 35235 |
| MER63 | 426 | 1.21% | 35235 |
| MLT1A | 398 | 1.13% | 35235 |
| MLT1F1 | 376 | 1.07% | 35235 |
| MER63A | 363 | 1.03% | 35235 |
| L1MA7 | 355 | 1.01% | 35235 |
| LTR2 | 337 | 0.96% | 35235 |
| MSTD | 332 | 0.94% | 35235 |
| LTR8 | 326 | 0.93% | 35235 |
| THE1C | 318 | 0.90% | 35235 |
| L1PB4 | 313 | 0.89% | 35235 |
| L1PA10 | 300 | 0.85% | 35235 |
| LTR9 | 298 | 0.85% | 35235 |
| LTR8A | 293 | 0.83% | 35235 |
| MER50 | 275 | 0.78% | 35235 |
| LTR9B | 274 | 0.78% | 35235 |
| LTR7B | 270 | 0.77% | 35235 |
| L1MA10 | 257 | 0.73% | 35235 |
| Charlie5 | 251 | 0.71% | 35235 |
| MLT1F | 248 | 0.70% | 35235 |
| LTR17 | 245 | 0.70% | 35235 |
| HERVH | 236 | 0.67% | 35235 |
| MLT1G1 | 225 | 0.64% | 35235 |
| SVA_A | 223 | 0.63% | 35235 |
| MLT1F2 | 208 | 0.59% | 35235 |
| MER4D1 | 204 | 0.58% | 35235 |
| L1MA1 | 201 | 0.57% | 35235 |
| SVA_B | 196 | 0.56% | 35235 |
| LTR7Y | 192 | 0.54% | 35235 |
| MER50B | 192 | 0.54% | 35235 |
| MSTC | 191 | 0.54% | 35235 |
| MER94 | 185 | 0.53% | 35235 |
| SST1 | 184 | 0.52% | 35235 |
| SVA_E | 175 | 0.50% | 35235 |
| MER81 | 173 | 0.49% | 35235 |
| MLT2D | 166 | 0.47% | 35235 |
| LTR33A | 164 | 0.47% | 35235 |
| MLT2B1 | 162 | 0.46% | 35235 |
| MER4A1 | 156 | 0.44% | 35235 |
| MLT2B4 | 151 | 0.43% | 35235 |
| LTR56 | 148 | 0.42% | 35235 |
| Harlequin | 148 | 0.42% | 35235 |
| MER68 | 146 | 0.41% | 35235 |
| LTR50 | 137 | 0.39% | 35235 |
| LTR14B | 133 | 0.38% | 35235 |
| MER115 | 132 | 0.37% | 35235 |
| MLT1E | 132 | 0.37% | 35235 |
| SVA_F | 131 | 0.37% | 35235 |
| MER57A-int | 131 | 0.37% | 35235 |
| MLT2A2 | 131 | 0.37% | 35235 |
| MLT2B3 | 126 | 0.36% | 35235 |
| ERVL-B4 | 126 | 0.36% | 35235 |
| LTR5_Hs | 125 | 0.35% | 35235 |
| MER66B | 124 | 0.35% | 35235 |
| L1PA12 | 122 | 0.35% | 35235 |
| GSATII | 120 | 0.34% | 35235 |
| MSR1 | 114 | 0.32% | 35235 |
| MLT1E1 | 107 | 0.30% | 35235 |
| LTR54B | 106 | 0.30% | 35235 |
| MER11C | 105 | 0.30% | 35235 |
| HERVK22 | 105 | 0.30% | 35235 |
| MER4D | 104 | 0.30% | 35235 |
| LTR48B | 104 | 0.30% | 35235 |
| HERV9 | 102 | 0.29% | 35235 |
| LTR52 | 100 | 0.28% | 35235 |
| BLACKJACK | 100 | 0.28% | 35235 |
| MLT1E3 | 94 | 0.27% | 35235 |
| LTR5B | 93 | 0.26% | 35235 |
| SVA_C | 93 | 0.26% | 35235 |
| MER51A | 91 | 0.26% | 35235 |
| LTR1B | 91 | 0.26% | 35235 |
| LTR2C | 90 | 0.26% | 35235 |
| HERVK14 | 89 | 0.25% | 35235 |
| MLT2B2 | 87 | 0.25% | 35235 |
| MER34B | 85 | 0.24% | 35235 |
| THE1B-int | 84 | 0.24% | 35235 |
| MER6A | 84 | 0.24% | 35235 |
| LTR2B | 83 | 0.24% | 35235 |
| HUERS-P3 | 82 | 0.23% | 35235 |
| LTR54 | 79 | 0.22% | 35235 |
| L1M3f | 72 | 0.20% | 35235 |
| MER92B | 69 | 0.20% | 35235 |
| LTR23 | 68 | 0.19% | 35235 |
| MER63D | 67 | 0.19% | 35235 |
| MER34C_{—} | 67 | 0.19% | 35235 |
| LTR6A | 67 | 0.19% | 35235 |
| HERVL18 | 66 | 0.19% | 35235 |
| LTR48 | 64 | 0.18% | 35235 |
| Cheshire | 63 | 0.18% | 35235 |
| LTR14C | 63 | 0.18% | 35235 |
| L1PA15-16 | 62 | 0.18% | 35235 |
| MER51B | 61 | 0.17% | 35235 |
| MER4A1-int | 61 | 0.17% | 35235 |
| HERVK9 | 60 | 0.17% | 35235 |
| MER34B-int | 60 | 0.17% | 35235 |
| MSTB2 | 58 | 0.16% | 35235 |
| LTR47A | 58 | 0.16% | 35235 |
| MER44C | 56 | 0.16% | 35235 |
| LTR22C | 56 | 0.16% | 35235 |
| MLT1E1A | 56 | 0.16% | 35235 |
| MER63C | 55 | 0.16% | 35235 |
| 7SK | 55 | 0.16% | 35235 |
| Charlie3 | 53 | 0.15% | 35235 |
| LTR29 | 52 | 0.15% | 35235 |
| LTR10B1 | 52 | 0.15% | 35235 |
| MLT1F-int | 51 | 0.14% | 35235 |

TABLE 17
List of Most Common Neighbor Repetitive DNA Sequence Families

| | | | | |
|---|---|---|---|---|
| AluY | AluSx | MLT1A0 | AluSg | L2 |
| AluSc | MSTA | AluSq | THE1B | MLT1A1 |
| L1PB1 | SVA_D | LTR7 | AluSp | THE1D |
| MSTB | LTR33 | LTR16C | MLT1E2 | ERVL-E |
| MSTB1 | MER63 | MLT1A | MLT1F1 | MER63A |
| L1MA7 | LTR2 | MSTD | LTR8 | THE1C |
| L1PB4 | L1PA10 | LTR9 | LTR8A | MER50 |
| LTR9B | LTR7B | L1MA10 | Charlie5 | MLT1F |
| LTR17 | HERVH | MLT1G1 | SVA_A | MLT1F2 |
| MER4D1 | L1MA1 | SVA_B | LTR7Y | MER50B |
| MSTC | MER94 | SST1 | SVA_E | MER81 |
| MLT2D | LTR33A | MLT2B1 | MER4A1 | MLT2B4 |
| LTR56 | Harlequin | MER68 | LTR50 | LTR14B |
| MER115 | MLT1E | SVA_F | MER57A-int | MLT2A2 |
| MLT2B3 | ERVL-B4 | LTR5_Hs | MER66B | L1PA12 |
| GSATII | MSR1 | MLT1E1 | LTR54B | MER11C |
| HERVK22 | MER4D | LTR48B | HERV9 | LTR52 |
| BLACKJACK | MLT1E3 | LTR5B | SVA_C | MER51A |
| LTR1B | LTR2C | HERVK14 | MLT2B2 | MER34B |
| THE1B-int | MER6A | LTR2B | HUERS-P3 | LTR54 |

TABLE 18
List of 569 Repetitive DNA Sequence Families (Status Biomarkers)

| | | | | |
|---|---|---|---|---|
| 5S | 7SK | 7SLRNA | AcHobo | ALR/Alpha |
| Alu | AluJ | AluJ/FLAM | AluJ/FRAM | AluJb |
| AluJo/FLAM | AluJo/FRAM | AluS | AluSc | AluSg |
| AluSg/x | AluSg1 | AluSp | AluSp/q | AluSq |
| AluSq/x | AluY | AluYa | AluYa5 | AluYa5/8 |
| AluYa8 | AluYb | AluYb9 | AluYc | AluYc3 |
| AluYc5 | AluYd | AluYd8 | AluYg | AluYg6 |
| AluYh | AluYh9 | Arthur1 | BLACKJACK | BSR/Beta |
| centr | Charlie1 | Charlie10 | Charlie1b | Charlie2 |
| Charlie2a | Charlie3 | Charlie4 | Charlie4a | Charlie5 |
| Charlie7 | Charlie8 | Charlie9 | Cheshire | CR1 |
| DNA | ERV | ERV1 | ERVK | ERVL |
| ERVL-B4 | ERVL-E | FAM | FLAM | FLAM_A |
| FordPrefect | FRAM | FRAM/FAM | GSAT | GSATII |
| HAL1 | HAL1b | Harlequin | HERV16 | HERV17 |
| HERV3 | HERV35I | HERV9 | HERVE | HERVFH21 |
| HERVH | HERVH48 | HERVI | HERVIP10F | HERVIP10FH |
| HERVK | HERVK11 | HERVK14 | HERVK22 | HERVK3 |
| HERVK9 | HERVL | HERVL-A1 | HERVL18 | HERVL40 |
| HERVP71A | HERVS71 | HSMAR1 | HSMAR2 | HUERS-P1 |
| HUERS-P2 | HUERS-P3 | HY1 | HY3 | K. standard. |
| | | | | SVA_A |
| K. standard. | K. standard. | K. standard. | K. standard. | K. standard. |
| SVA_B | SVA_C | SVA_D | SVA_E | SVA_F |
| Kanga1 | Kanga2_a | L1 | L1HS | L1M |
| L1M1 | L1M2 | L1M3 | L1M3b | L1M3c |
| L1M3d | L1M3e | L1M3f | L1M4 | L1M4b |
| L1M4c | L1M5 | L1MA1 | L1MA10 | L1MA2 |
| L1MA3 | L1MA4A | L1MA5 | L1MA5A | L1MA6 |
| L1MA7 | L1MA8 | L1MA9 | L1MB1 | L1MB2 |
| L1MB3 | L1MB4 | L1MB5 | L1MB7 | L1MB8 |
| L1MC1 | L1MC2 | L1MC3 | L1MC4 | L1MC4_3endX |
| L1MC4a | L1MC5 | L1MCa | L1MCb | L1MCc |
| L1MD | L1MD1 | L1MD2 | L1MD3 | L1MDa |
| L1ME2 | L1ME3 | L1ME3A | L1ME4a | L1MEa |
| L1MEb | L1MEd | L1MEe | L1P | L1P1 |
| L1P2 | L1P3 | L1P4 | L1P4a | L1P5 |
| L1PA10 | L1PA11 | L1PA12 | L1PA13 | L1PA14 |
| L1PA15 | L1PA15-16 | L1PA16 | L1PA17 | L1PA2 |
| L1PA3 | L1PA4 | L1PA5 | L1PA6 | L1PA7 |
| L1PA8 | L1PA8A | L1PB | L1PB1 | L1PB2 |
| L1PB3 | L1PB4 | L1PBa | L1PBa1 | L2 |
| L3 | L3b | LINE | Looper | LOR1-int |
| LOR1b | LTR | LTR1 | LTR10B | LTR10B1 |
| LTR10C | LTR10D | LTR10F | LTR12 | LTR12_{—} |
| LTR12B | LTR12C | LTR12D | LTR12E | LTR13 |
| LTR13_{—} | LTR13A | LTR14 | LTR14B | LTR14C |
| LTR16A | LTR16A1 | LTR16B | LTR16C | LTR16D |
| LTR17 | LTR18A | LTR18B | LTR19A | LTR1B |
| LTR1C | LTR1D | LTR2 | LTR22 | LTR22C |
| LTR23 | LTR24 | LTR24C | LTR26 | LTR26B |
| LTR26E | LTR27 | LTR27B | LTR28 | LTR29 |
| LTR2B | LTR2C | LTR3 | LTR30 | LTR32 |
| LTR33 | LTR33A | LTR34 | LTR35 | LTR36 |
| LTR37A | LTR37B | LTR38 | LTR39 | LTR3A |
| LTR3B | LTR40a | LTR40b | LTR41 | LTR43 |
| LTR45 | LTR45B | LTR45C | LTR46 | LTR47A |
| LTR48 | LTR48B | LTR49 | LTR49-int | LTR5 |
| LTR5_Hs | LTR50 | LTR51 | LTR52 | LTR53 |
| LTR54 | LTR54B | LTR56 | LTR5A | LTR5B |
| LTR62 | LTR65 | LTR66 | LTR67 | LTR6A |
| LTR6B | LTR7 | LTR72 | LTR75 | LTR7B |
| LTR7Y | LTR8 | LTR8A | LTR9 | LTR9B |
| MADE1 | MADE2 | MaLR | MARNA | MER1_type |
| MER1_type? | MER101-int | MER102b | MER103 | MER104 |
| MER105 | MER106A | MER106B | MER110 | MER110A |
| MER112 | MER113 | MER115 | MER117 | MER119 |
| MER11A | MER11B | MER11C | MER11D | MER121 |
| MER1A | MER1B | MER2 | MER2_type | MER20 |
| MER20B | MER21-int | MER21A | MER21B | MER21C |
| MER2B | MER3 | MER30 | MER31-int | MER31A |
| MER31B | MER33 | MER34 | MER34B | MER34B-int |
| MER34C | MER34C_{—} | MER39 | MER39B | MER4-int |
| MER41A | MER41B | MER41B-int | MER41C | MER41D |
| MER41E | MER44A | MER44B | MER44C | MER44D |
| MER45 | MER45A | MER45B | MER45C | MER45R |
| MER46A | MER46B | MER46C | MER48 | MER49 |
| MER4A | MER4A1 | MER4A1_{—} | MER4A1-int | MER4B |
| MER4B-int | MER4C | MER4D | MER4D0 | MER4D1 |
| MER4E | MER4E1 | MER50 | MER50-int | MER50B |
| MER51A | MER51B | MER51B-int | MER52A | MER52C |
| MER52D | MER53 | MER54A | MER54B | MER57A |
| MER57A-int | MER57B | MER57B-int | MER58 | MER58A |
| MER58B | MER58C | MER5A | MER5A1 | MER5B |
| MER5C | MER6 | MER61A | MER61A-int | MER61B |
| MER63 | MER63A | MER63D | MER65-int | MER65A |
| MER65A-int | MER65C | MER65D | MER66A | MER66B |
| MER66C | MER67A | MER67B | MER67C | MER67D |
| MER68 | MER69A | MER69B | MER6A | MER70A |
| MER72 | MER74A | MER74B | MER75 | MER76 |
| MER77 | MER7A | MER7C | MER8 | MER81 |
| MER82 | MER83 | MER84 | MER85 | MER87 |
| MER89 | MER9 | MER90 | MER90a | MER91 |
| MER91A | MER91B | MER91C | MER92B | MER93a |
| MER93B | MER93b | MER94 | MER96 | MER96B |
| MER97c | MIR | MIR3 | MIRb | MIRm |
| MLT-int | MLT1A | MLT1A0 | MLT1A0-int | MLT1A1 |
| MLT1B | MLT1C | MLT1D | MLT1D-int | MLT1E |
| MLT1E1 | MLT1E1A | MLT1E2 | MLT1E3 | MLT1F |
| MLT1F-int | MLT1F1 | MLT1F2 | MLT1G1 | MLT1G3 |
| MLT1H | MLT1H-int | MLT1H1 | MLT1H2 | MLT1I |
| MLT1J | MLT1J1 | MLT1J2 | MLT1K | MLT1L |
| MLT2A1 | MLT2A2 | MLT2B1 | MLT2B2 | MLT2B3 |
| MLT2B4 | MLT2C1 | MLT2C2 | MLT2D | MLT2E |
| MLT2F | MSR1 | MSTA | MSTA-int | MSTB |
| MSTB-int | MSTB1 | MSTB2 | MSTC | MSTD |
| MuDR | ORSL | Other | PABL_A | PABL_B |
| PiggyBac | PRIMA4_LTR | PRIMA4-int | REP522 | Ricksha |
| RNA | rRNA | Satellite | SATR1 | SATR2 |
| scRNA | SINE | snRNA | srpRNA | SST1 |
| SVA | TAR1 | Tc2 | telo | THE1A |
| THE1B | THE1B-int | THE1C | THE1D | Tigger1 |
| Tigger2 | Tigger2a | Tigger3b | Tigger4(Zombi) | Tigger5 |
| Tigger5a | Tigger5b | Tigger6 | Tigger6a | Tigger7 |
| Tigger8 | Tip100 | tRNA | U1 | U13 |
| U2 | U6 | Zaphod | Zaphod2 | |

### B. Nucleic Acids

There are a variety of molecules disclosed herein that are nucleic acid based, including, for example, riboswitches, aptamers, and nucleic acids that encode riboswitches and aptamers. The disclosed nucleic acids can be made up of for example, nucleotides, nucleotide analogs, or nucleotide substitutes. Non-limiting examples of these and other molecules are discussed herein. It is understood that for example, when a vector is expressed in a cell that the expressed mRNA will typically be made up of A, C, G, and U. Likewise, it is understood that if a nucleic acid molecule is introduced into a cell or cell environment through for example exogenous delivery, it is advantageous that the nucleic acid molecule be made up of nucleotide analogs that reduce the degradation of the nucleic acid molecule in the cellular environment.

So long as their relevant function is maintained, riboswitches, aptamers, expression platforms and any other oligonucleotides and nucleic acids can be made up of or include modified nucleotides (nucleotide analogs). Many modified nucleotides are known and can be used in oligonucleotides and nucleic acids. A nucleotide analog is a nucleotide which contains some type of modification to either the base, sugar, or phosphate moieties. Modifications to the base moiety would include natural and synthetic modifications of A, C, G, and T/U as well as different purine or pyrimidine bases, such as uracil-5-yl, hypoxanthin-9-yl (I), and 2-aminoadenin-9-yl. A modified base includes but is not limited to 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Another modified base contains one or more of the 2′-O,4′-C-methylene-β-D-ribofuranosyl nucleosides which are known as locked nucleic acid (LNA™) monomers (Petersen and Wengel, Trends Biotech 21:74-81, 2003). Additional base modifications can be found for example in U.S. Pat. No. 3,687,808, Englisch et al., Angewandte Chemie, International Edition, 1991, 30, 613, and Sanghvi, Y. S., Chapter 15, Antisense Research and Applications, pages 289-302, Crooke, S. T. and Lebleu, B. ed., CRC Press, 1993. Certain nucleotide analogs, such as 5-substituted pyrimidines, 6-azapyrimidines and N-2, N-6 and O-6 substituted purines, including 2-aminopropyladenine, 5-propynyluracil and 5-propynylcytosine. 5-methylcytosine can increase the stability of duplex formation. Other modified bases are those that function as universal bases. Universal bases include 3-nitropyrrole and 5-nitroindole. Universal bases substitute for the normal bases but have no bias in base pairing. That is, universal bases can base pair with any other base. Base modifications often can be combined with for example a sugar modification, such as 2′-β-methoxyethyl, to achieve unique properties such as increased duplex stability. There are numerous United States patents such as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; and 5,681,941, which detail and describe a range of base modifications. Each of these patents is herein incorporated by reference in its entirety, and specifically for their description of base modifications, their synthesis, their use, and their incorporation into oligonucleotides and nucleic acids.

LNA™ monomers are a class of nucleic acid analogues in which the ribose ring is “locked” into the ideal conformation for base stacking and backbone pre-organization and can be used just like a regular nucleotide. The nucleic acid contains a methylene bridge connecting the 2′-O and the 4′-C. The “locked” structure increases the stability of oligonucleotides by means of increasing the melting temperature (Kaur et al. Biochemistry 45:7347-55, 2006). LNA™ can be used for a variety of molecular biology techniques. Locked nucleic acids can be used for but are not limited to microarrays, FISH probes, real-time PCR probes, small RNA research, SNP genotyping, mRNA antisense oligonucleotides, allele-specific PCR, RNAi, DNAzymes, fluorescence polarization probes, gene repair/exon skipping, splice variant detection and comparative genome hybridization.

Nucleotide analogs can also include modifications of the sugar moiety. Modifications to the sugar moiety would include natural modifications of the ribose and deoxyribose as well as synthetic modifications. Sugar modifications include but are not limited to the following modifications at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl can be substituted or unsubstituted C1 to C10, alkyl or C2 to C10 alkenyl and alkynyl. 2′ sugar modifications also include but are not limited to —O[(CH₂)n O]m CH₃, —O(CH₂)n OCH₃, —O(CH₂)n NH₂, —O(CH₂)n CH₃, —O(CH₂)n —ONH₂, and —O(CH₂)nON[(CH₂)n CH₃)]₂, where n and m are from 1 to about 10.

Other modifications at the 2′ position include but are not limited to: C1 to C10 lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂ CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications can also be made at other positions on the sugar, particularly the 3′ position of the sugar on the 3′ terminal nucleotide or in 2′-5′ linked oligonucleotides and the 5′ position of 5′ terminal nucleotide. Modified sugars would also include those that contain modifications at the bridging ring oxygen, such as CH₂ and S. Nucleotide sugar analogs can also have sugar mimetics such as cyclobutyl moieties in place of the pentofuranosyl sugar. There are numerous United States patents that teach the preparation of such modified sugar structures such as U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, each of which is herein incorporated by reference in its entirety, and specifically for their description of modified sugar structures, their synthesis, their use, and their incorporation into nucleotides, oligonucleotides and nucleic acids.

Nucleotide analogs can also be modified at the phosphate moiety. Modified phosphate moieties include but are not limited to those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. It is understood that these phosphate or modified phosphate linkages between two nucleotides can be through a 3′-5′ linkage or a 2′-5′ linkage, and the linkage can contain inverted polarity such as 3′-5′ to 5′-3′ or 2′-5′ to 5′-2′. Various salts, mixed salts and free acid forms are also included. Numerous United States patents teach how to make and use nucleotides containing modified phosphates and include but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, each of which is herein incorporated by reference its entirety, and specifically for their description of modified phosphates, their synthesis, their use, and their incorporation into nucleotides, oligonucleotides and nucleic acids.

It is understood that nucleotide analogs need only contain a single modification, but can also contain multiple modifications within one of the moieties or between different moieties.

Nucleotide substitutes are molecules having similar functional properties to nucleotides, but which do not contain a phosphate moiety, such as peptide nucleic acid (PNA). Nucleotide substitutes are molecules that will recognize and hybridize to (base pair to) complementary nucleic acids in a Watson-Crick or Hoogsteen manner, but which are linked together through a moiety other than a phosphate moiety. Nucleotide substitutes are able to conform to a double helix type structure when interacting with the appropriate target nucleic acid.

Nucleotide substitutes can also include nucleotides or nucleotide analogs that have had the phosphate moiety and/or sugar moieties replaced. Nucleotide substitutes do not contain a standard phosphorus atom. Substitutes for the phosphate can be for example, short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH2 component parts. Numerous United States patents disclose how to make and use these types of phosphate replacements and include but are not limited to U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference its entirety, and specifically for their description of phosphate replacements, their synthesis, their use, and their incorporation into nucleotides, oligonucleotides and nucleic acids.

It is also understood in a nucleotide substitute that both the sugar and the phosphate moieties of the nucleotide can be replaced, by for example an amide type linkage (aminoethylglycine) (PNA). U.S. Pat. Nos. 5,539,082; 5,714,331; and 5,719,262 teach how to make and use PNA molecules, each of which is herein incorporated by reference. (See also Nielsen et al., Science 254:1497-1500 (1991)).

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety. (Letsinger et al., Proc. Natl. Acad. Sci. USA, 1989, 86, 6553-6556). There are many varieties of these types of molecules available in the art and available herein.

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA.

The Hoogsteen face includes the N7 position and reactive groups (NH2 or O) at the C6 position of purine nucleotides.

Oligonucleotides and nucleic acids can be comprised of nucleotides and can be made up of different types of nucleotides or the same type of nucleotides. For example, one or more of the nucleotides in an oligonucleotide can be ribonucleotides, 2′-O-methyl ribonucleotides, or a mixture of ribonucleotides and 2′-O-methyl ribonucleotides; about 10% to about 50% of the nucleotides can be ribonucleotides, 2′-O-methyl ribonucleotides, or a mixture of ribonucleotides and 2′-O-methyl ribonucleotides; about 50% or more of the nucleotides can be ribonucleotides, 2′-O-methyl ribonucleotides, or a mixture of ribonucleotides and 2′-O-methyl ribonucleotides; or all of the nucleotides are ribonucleotides, 2′-O-methyl ribonucleotides, or a mixture of ribonucleotides and 2′-O-methyl ribonucleotides. Such oligonucleotides and nucleic acids can be referred to as chimeric oligonucleotides and chimeric nucleic acids.

It is understood that as discussed herein the use of the terms homology and identity mean the same thing as similarity. Thus, for example, if the use of the word homology is used between two sequences (non-natural sequences, for example) it is understood that this is not necessarily indicating an evolutionary relationship between these two sequences, but rather is looking at the similarity or relatedness between their nucleic acid sequences. Many of the methods for determining homology between two evolutionarily related molecules are routinely applied to any two or more nucleic acids or proteins for the purpose of measuring sequence similarity regardless of whether they are evolutionarily related or not.

In general, it is understood that one way to define any known variants and derivatives or those that might arise, of the disclosed riboswitches, aptamers, expression platforms, genes and proteins herein, is through defining the variants and derivatives in terms of homology to specific known sequences. This identity of particular sequences disclosed herein is also discussed elsewhere herein. In general, variants of sequences herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to a stated sequence or a native sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison can be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment. It is understood that any of the methods typically can be used and that in certain instances the results of these various methods can differ, but the skilled artisan understands if identity is found with at least one of these methods, the sequences would be said to have the stated identity.

For example, as used herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages).

The term hybridization typically means a sequence driven interaction between at least two nucleic acid molecules, such as a primer or a probe and a riboswitch or a gene. Sequence driven interaction means an interaction that occurs between two nucleotides or nucleotide analogs or nucleotide derivatives in a nucleotide specific manner. For example, G interacting with C and A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize.

Parameters for selective hybridization between two nucleic acid molecules are well known to those of skill in the art. For example, in some embodiments selective hybridization conditions can be defined as stringent hybridization conditions. For example, stringency of hybridization is controlled by both temperature and salt concentration of either or both of the hybridization and washing steps. For example, the conditions of hybridization to achieve selective hybridization can involve hybridization in high ionic strength solution (6×SSC or 6×SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art (Sambrook et al., Molecular Cloning: A Laboratory Manual, 2nd Ed., Cold Spring Harbor Laboratory, Cold Spring Harbor, N.Y., 1989; Kunkel et al. Methods Enzymol. 1987:154:367, 1987 which is herein incorporated by reference for material at least related to hybridization of nucleic acids). A preferable stringent hybridization condition for a DNA:DNA hybridization can be at about 68° C. (in aqueous solution) in 6×SSC or 6×SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

Another way to define selective hybridization is by looking at the amount (percentage) of one of the nucleic acids bound to the other nucleic acid. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the limiting nucleic acid is bound to the non-limiting nucleic acid. Typically, the non-limiting nucleic acid is in for example, 10 or 100 or 1000 fold excess. This type of assay can be performed at under conditions where both the limiting and non-limiting nucleic acids are for example, 10 fold or 100 fold or 1000 fold below their k_{d}, or where only one of the nucleic acid molecules is 10 fold or 100 fold or 1000 fold or where one or both nucleic acid molecules are above their k_{d}.

Another way to define selective hybridization is by looking at the percentage of nucleic acid that gets enzymatically manipulated under conditions where hybridization is required to promote the desired enzymatic manipulation. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the nucleic acid is enzymatically manipulated under conditions which promote the enzymatic manipulation, for example if the enzymatic manipulation is DNA extension, then selective hybridization conditions would be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the nucleic acid molecules are extended. Preferred conditions also include those suggested by the manufacturer or indicated in the art as being appropriate for the enzyme performing the manipulation.

Just as with homology, it is understood that there are a variety of methods herein disclosed for determining the level of hybridization between two nucleic acid molecules. It is understood that these methods and conditions can provide different percentages of hybridization between two nucleic acid molecules, but unless otherwise indicated meeting the parameters of any of the methods would be sufficient. For example if 80% hybridization was required and as long as hybridization occurs within the required parameters in any one of these methods it is considered disclosed herein.

It is understood that those of skill in the art understand that if a composition or method meets any one of these criteria for determining hybridization either collectively or singly it is a composition or method that is disclosed herein.

1. Probes and Primers

Disclosed are compositions including primers and probes, which are capable of interacting with the disclosed nucleic acids such as status biomarkers, DNA fragments, repetitive DNA sequences, unique sequences, PCR amplicons, and probe binding sequences. In certain embodiments the primers are used to support DNA amplification reactions. Typically the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner. Typically the disclosed primers hybridize with the disclosed nucleic acids or region of the nucleic acids or they hybridize with the complement of the nucleic acids or complement of a region of the nucleic acids.

Probe for biomarkers can be designed in any suitable manner. Examples of methods and techniques for designing probes are described herein, but any other methods and techniques can be used. Useful probes can be specific for particular biomarkers, loci, families of biomarkers, families of loci, etc. Sequence analysis of biomarker and loci sequences (such as nucleic acid regions containing CpG islands and CpG islets) can be used to identify specific and/or selective probes. Particularly useful probes can be complementary to unique sequences in biomarkers and loci of interest or to characteristic or consensus sequences in biomarker and locus families.

The size of the primers or probes for interaction with the nucleic acids in certain embodiments can be any size that supports the desired enzymatic manipulation of the primer, such as DNA amplification or the simple hybridization of the probe or primer. A typical primer or probe would be at least 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

In other embodiments a primer or probe can be less than or equal to 6, 7, 8, 9, 10, 11, 12 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

The disclosed methods involve the use of probes and primers. For example, probes for status biomarkers are used to capture, detect, measure, and/or assess status biomarkers. These and other probes can be designed and made using any suitable techniques. Many such techniques are known in the art. The examples and other description herein provide examples of the design of probes and of features useful to the probes to be used in the disclosed methods. The disclosed probes can be used, for example, to detect the level of the status biomarkers by using, for example, an array of probes specific for the status biomarkers. In some forms, the array of probes can be, for example, a microarray.

Useful forms of the disclosed probes can be complementary to, and/or specific for, any sequence in a status biomarker. Such complementary sequences in status biomarkers can be referred to as probe binding sites. Particularly useful target sequences for probes are unique sequences and repetitive DNA sequences. Useful probes for unique sequences can have a sequence of sufficient length and having a nucleotide sequence distinctive enough to hybridize uniquely in the genome at the unique sequence. For example, nucleic acid sequences of or at least 15, 20, 25, 30, 35, 40, 45, 50, 55, 60, 65, 70, 75, 80, 85, 90, 95, or 100 nucleotides in length can be used as probes for unique sequences. Unique sequences can be identified by, for example, analysis of a genome sequence or by analysis of probe hybridization. Probes for repetitive DNA sequences and other targets can have any useful length. For example, nucleic acid sequences of or at least 10, 15, 20, 25, 30, 35, 40, 45, 50, 55, 60, 65, 70, 75, 80, 85, 90, 95, or 100 nucleotides in length can be used as probes.

Probes can be specific for probe binding sites in status biomarkers. In some forms, the one or more of the status biomarkers can comprise a probe binding site, wherein the probe binding site of the one or more of the status biomarkers is specific for a probe. Probe binding sites can be, for example, all or a portion of a unique sequence in the status biomarker. In some forms, one or more of the probes can be specific for a repetitive DNA sequence locus, wherein the repetitive DNA sequence locus comprises one or more repetitive DNA sequences, wherein independently for each of the one or more of the probes one or more of the repetitive DNA sequences belongs to a family of repetitive DNA sequences listed in, for example, Table 1, Table 12, or Table 13. In some forms, each probe can be specific for a repetitive DNA sequence locus, wherein independently for each probe one or more of the repetitive DNA sequences belongs to a family of repetitive DNA sequences such as the repetitive DNA sequence families listed in, for example, Table 1, Table 12, or Table 13.

In some forms, one or more of the probes can be specific for a repetitive DNA sequence locus, wherein the repetitive DNA sequence locus comprises one or more repetitive DNA sequences, wherein for one or more of the probes one or more of the repetitive DNA sequences is an interspersed repeat element. In some forms, each probe can be specific for a repetitive DNA sequence locus, wherein for each probe one or more of the repetitive DNA sequences is an interspersed repeat element.

Primers can be used in the disclosed methods to replicate and/or amplify nucleic acids. For example, primers for PCR can be used to amplify genomic sequences and sequences of status biomarkers. Primers can also be used for other replication and replication techniques, such a multiple displacement amplification and replication-based nucleic acid sequencing techniques. Many such techniques are known and principles and techniques for design of primers for use in such techniques are known and can be used for the disclosed primers and methods.

In some forms of the disclosed methods, part or all of a status biomarker can be replicated and/or amplified as a PCR amplicon. In some forms, one or more of the status biomarkers can comprise a PCR amplicon. A PCR amplicon is a region of nucleic acid including and between the binding sites of PCR primers. PCR amplicons can be said to be defined by the binding sites of the primers and by the primers themselves. In some forms, the PCR amplicon of each of the one or more of the status biomarkers can be defined by a first primer specific for a single one of the status biomarkers and a second primer. A primer specific for a status biomarker refers to a primer that can bind to a sequence in, and prime replication of, the status biomarker. A primer specific for a repetitive DNA sequence refers to a primer that can bind to a sequence in, and prime replication of, the repetitive DNA sequence. In some forms, the PCR amplicon of each of the one or more of the status biomarkers can be defined by the same first primer specific for a first type of repetitive DNA sequence and a second primer, wherein the second primer is specific for a second type of repetitive DNA sequence, wherein the second primer is the same for some and different for some of the one or more of the status biomarkers. In some forms, the first primer can be specific for one of the families of repetitive DNA sequences listed in Table 16 or 17, wherein independently for each of the one or more of the status biomarkers the second primer is specific for a family of repetitive DNA sequences such as the repetitive DNA sequence families listed in, for example, Table 1, Table 12, or Table 13. A primer specific for a family of repetitive DNA sequence refers to a primer that can bind to a sequence in, and prime replication of, one or more repetitive DNA sequences in the family of repetitive DNA sequences.

The presence of repetitive DNA sequences belonging to different families of repetitive DNA sequences in the same, for example, status biomarker or repetitive DNA sequence locus can facilitate some of the forms of the disclosed methods. For example, the different repetitive DNA sequences can be used to define a PCR amplicon by, for example, using primers specific for two of the different repetitive DNA sequences.

In some forms, detecting the level of the status biomarkers can be accomplished via, for example, amplifying the processed DNA and determining the ratio of cytosine to thymidine in the amplified DNA and converting the ratio to the level of methylated forms of the status biomarkers. In some forms, the processed DNA can be amplified via, for example, PCR amplification of the status biomarkers using primers specific for the status biomarkers.

In some forms, detecting the level of the status biomarkers can be accomplished via, for example, PCR amplification of the status biomarkers using primers specific for the status biomarkers. In some forms, the PCR amplification can be quantitative PCR. In some forms, the PCR amplification can be nanoliter-microarray quantitative PCR.

Probes can also be used to capture status biomarkers and sequences derived from status biomarkers. Such probes can be referred to as capture probes, status biomarker capture probes, or status biomarker probes. In some forms, treating the DNA sample can be accomplished by, for example, capturing status biomarker DNA fragments. In some forms, the status biomarker DNA fragments can be captured by, for example, binding DNA fragments in the DNA sample to status biomarker probes attached to a support. In some forms, one or more of the status biomarker probes can specifically hybridize to one or more repetitive DNA sequences. In some forms, each of the status biomarker probes can specifically hybridize to one or more repetitive DNA sequences. Such probes can be specific for specific repetitive DNA sequences. Such probes can also be specific for a group or family of repetitive DNA sequences or a group or family of status biomarkers. For example, one or more of the status biomarker probes can comprise degenerate sequence representing a consensus sequence for a family of repetitive DNA sequences. In some forms, the family of repetitive DNA sequences can be a family of repetitive DNA sequences such as the repetitive DNA sequence families listed in, for example, Table 1, Table 12, or Table 13. In some forms, the one or more of the status biomarker probes can comprise different degenerate sequences each representing a consensus sequence for a different one of the families of repetitive DNA sequences such as the repetitive DNA sequence families listed in, for example, Table 1, Table 12, or Table 13. In some forms, the one or more of the status biomarker probes can comprise at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, or 135 different degenerate sequences each representing a different consensus sequence for a different one of the families of repetitive DNA sequences such as the repetitive DNA sequence families listed in, for example, Table 1, Table 12, or Table 13. The families of repetitive DNA sequences can be selected for in any manner, including by selecting the first at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, or 135 families in rank order. In some forms, the support can comprise, for example, gel, a bead, a magnetic bead, a plate, a slide, a surface, or a microparticle. In some forms, DNA not captured can be separated from the captured status biomarker DNA fragments. In some forms, the sequencing can be a form of SMRT sequencing.

In some forms, the method can further comprise, after capturing status biomarker DNA fragments and prior to sequencing the captured status biomarker DNA fragments, releasing the captured status biomarker DNA fragments and recapturing the released status biomarker DNA fragments. In some forms, the status biomarker DNA fragments can be recaptured by binding DNA fragments in the DNA sample to secondary status biomarker probes attached to a support. In some forms, one or more of the status biomarker probes can specifically hybridize to one or more repetitive DNA sequences, wherein the one or more of the status biomarker probes comprises degenerate sequence representing a consensus sequence for a family of repetitive DNA sequences. In some forms, each of the status biomarker probes can specifically hybridize to one or more repetitive DNA sequences, wherein each of the status biomarker probes comprises degenerate sequence representing a consensus sequence for a family of repetitive DNA sequences. In some forms, the family of repetitive DNA sequences can be a family of repetitive DNA sequences such as the repetitive DNA sequence families listed in, for example, Table 16 and Table 17. In some forms, the family of repetitive DNA sequences can be a family of repetitive DNA sequences listed in Table 16 or 17. For example, the family of repetitive DNA sequences can be the AluY, AluSx, AluSp, AluSg, or AluSc family of repetitive DNA sequences. In some forms, the one or more of the status biomarker probes can comprise different degenerate sequences each representing a consensus sequence for a different one of the families of repetitive DNA sequences such as the repetitive DNA sequence families listed in, for example, Table 16 and Table 17. In some forms, the one or more of the status biomarker probes can comprise different degenerate sequences each representing a consensus sequence for a different one of the families of repetitive DNA sequences listed in Table 16 or 17, such as AluY, AluSx, AluSp, AluSg, or AluSc.

In some forms, status biomarker probes can be produced by, for example, selecting a subset of repetitive DNA sequence loci from a set of repetitive DNA sequence loci, generating a set of status biomarker probe sequences, and synthesizing one or more status biomarker probes. In some forms, the method for producing status biomarker probes can further comprise selecting one or more additional subsets of repetitive DNA sequence loci each from a different additional set of repetitive DNA sequence loci, generating one or more additional sets of status biomarker probe sequences each based on one of the one or more additional subsets, and synthesizing one or more additional status biomarker probes, wherein each additional status biomarker probe has the sequence of one of the additional status biomarker probe sequences. In some forms, the repetitive DNA sequence loci in the set of repetitive DNA sequence loci can belong to a single one of the families of repetitive DNA sequence such as the repetitive DNA sequence families listed in, for example, Table 1, Table 12, or Table 13, wherein the subset of repetitive DNA sequence loci can be selected by identifying those repetitive DNA sequence loci that comprise a repetitive DNA sequence belonging to one of the families of repetitive DNA sequences such as the repetitive DNA sequence families listed in, for example, Table 16 and Table 17. In some forms, the repetitive DNA sequence loci in each additional set of repetitive DNA sequence loci can independently belong to a different single one of the families of repetitive DNA sequence such as the repetitive DNA sequence families listed in, for example, Table 1, Table 12, or Table 13, wherein the repetitive DNA sequence loci in the set of repetitive DNA sequence loci and in each additional set of repetitive DNA sequence loci belong to different families of repetitive DNA sequence.

In some forms, each status biomarker probe sequence in a set can have a length of, for example, 15, 20, 25, 30, 35, 40, 45, 50, 55, 60, 65, 70, 75, 80, 85, 90, 95, or 100 bases or more. In some forms, each status biomarker probe represented in the set of status biomarker probe sequences can hybridize to, for example, at least 1%, 2%, 3%, 4%, 5%, 6%, 7%, 8%, 9%, 10%, 11%, 12%, 13%, 14%, 15% of the repetitive DNA sequence loci in the selected subset of repetitive DNA sequence loci. In some forms, each status biomarker probe can have the sequence of one of the generated status biomarker probe sequences.

In some forms, the set of status biomarker probe sequences can include, for example, any range of from 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 32, 34, 35, 36, 38, 40, 42, 44, 45, 46, 48, 50, 55, 60, 65, 70, 75, 80, 85, 90, 95, 100, 110, 120, 130, 140, 150, 160, 170, 180, 190, or 200 to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 32, 34, 35, 36, 38, 40, 42, 44, 45, 46, 48, 50, 55, 60, 65, 70, 75, 80, 85, 90, 95, 100, 110, 120, 130, 140, 150, 160, 170, 180, 190, or 200 status biomarker probe sequences. In some forms, the set of status biomarker probe sequences can comprise from 5 to 100 status biomarker probe sequences. In some forms, the set of status biomarker probe sequences can comprise from 10 to 100 status biomarker probe sequences. In some forms, one or more of the additional sets of status biomarker probe sequences each can comprise from 1 to 100 status biomarker probe sequences. In some forms, the one or more additional sets of status biomarker probe sequences each can comprise from 5 to 100 status biomarker probe sequences. In some forms, the one or more additional sets of status biomarker probe sequences each can comprise from 10 to 100 status biomarker probe sequences.

### C. Solid Supports

The disclosed methods and compositions can use supports. For example, probes and primers can be attached or associated with supports for use in the disclosed methods. Such probe and primer associated supports can take the form of, for example, arrays and microarrays. Solid supports are solid-state substrates or supports with which molecules (such as probes and primers) can be associated. Probes, primers, and other molecules can be associated with solid supports directly or indirectly. For example, probes can be bound to the surface of a solid support or associated with capture agents (e.g., oligonucleotides or molecules that bind a probe) immobilized on solid supports. As another example, probes can be bound to the surface of a solid support or associated with oligonucleotides immobilized on solid supports. An array is a solid support to which multiple probes, primers, or other molecules have been associated in an array, grid, or other organized pattern.

Solid-state substrates for use in solid supports can include any solid material with which components can be associated, directly or indirectly. This includes materials such as gel, acrylamide, agarose, cellulose, nitrocellulose, glass, gold, polystyrene, polyethylene vinyl acetate, polypropylene, polymethacrylate, polyethylene, polyethylene oxide, polysilicates, polycarbonates, teflon, fluorocarbons, nylon, silicon rubber, polyanhydrides, polyglycolic acid, polylactic acid, polyorthoesters, functionalized silane, polypropylfumerate, collagen, glycosaminoglycans, and polyamino acids. Solid-state substrates can have any useful form including thin film, membrane, bottles, dishes, plates, slides, fibers, woven fibers, shaped polymers, chromatography matrix, particles, magnetic particles, beads, magnetic beads, microparticles, magnetic microparticles, nanoparticles, magnetic nanoparticles, or a combination. Solid-state substrates and solid supports can be porous or non-porous. A chip is a rectangular or square small piece of material. Useful forms for solid-state substrates are thin films, beads, or chips. A useful form for a solid-state substrate is a microtiter dish. In some embodiments, a multiwell glass slide can be employed.

An array can include a plurality of probes, other molecules, compounds or primers immobilized at identified or predefined locations on the solid support. Each predefined location on the solid support generally has one type of component (that is, all the components at that location are the same). Alternatively, multiple types of components can be immobilized in the same predefined location on a solid support. Each location will have multiple copies of the given components. The spatial separation of different components on the solid support allows separate detection and identification.

Although useful, it is not required that the solid support be a single unit or structure. A set of probes, other molecules, compounds and/or primers can be distributed over any number of solid supports. For example, at one extreme, each component can be immobilized in a separate reaction tube or container, or on separate beads or microparticles.

Methods for immobilization of oligonucleotides to solid-state substrates are well established. Oligonucleotides, including address probes and detection probes, can be coupled to substrates using established coupling methods. For example, suitable attachment methods are described by Pease et al., Proc. Natl. Acad. Sci. USA 91(11):5022-5026 (1994), and Khrapko et al., Mol Biol (Mosk) (USSR) 25:718-730 (1991). A method for immobilization of 3′-amine oligonucleotides on casein-coated slides is described by Stimpson et al., Proc. Natl. Acad. Sci. USA 92:6379-6383 (1995). A useful method of attaching oligonucleotides to solid-state substrates is described by Guo et al., Nucleic Acids Res. 22:5456-5465 (1994).

Each of the components (for example, probes, primers, or other molecules) immobilized on the solid support can be located in a different predefined region of the solid support. The different locations can be different reaction chambers. Each of the different predefined regions can be physically separated from each other of the different regions. The distance between the different predefined regions of the solid support can be either fixed or variable. For example, in an array, each of the components can be arranged at fixed distances from each other, while components associated with beads will not be in a fixed spatial relationship. In particular, the use of multiple solid support units (for example, multiple beads) will result in variable distances.

Components can be associated or immobilized on a solid support at any density. Components can be immobilized to the solid support at a density exceeding 400 different components per cubic centimeter. Arrays of components can have any number of components. For example, an array can have at least 1,000 different components immobilized on the solid support, at least 10,000 different components immobilized on the solid support, at least 100,000 different components immobilized on the solid support, or at least 1,000,000 different components immobilized on the solid support.

### D. Samples

Any nucleic acid sample can be used with the disclosed methods. Examples of suitable nucleic acid samples include DNA samples, genomic samples, mRNA samples, cDNA samples, nucleic acid libraries (including cDNA and genomic libraries), whole cell samples, culture samples, tissue samples, bodily fluids, biopsy samples, or a combination. Numerous other sources of nucleic acid samples are known or can be developed and any can be used with the disclosed method. Generally, it is useful to use a genomic sample from cells, tissues, subjects, that are relevant to the status being assessed.

The source, identity, and preparation of many such nucleic acid samples are known. The nucleic acid sample can be, for example, a nucleic acid sample from one or more cells, tissue, skin, lung, head, neck, prostate, breast, ovary, brain, liver, stomach, intestine, kidney, testicle, cervix, uterus, spleen, bone, throat, esophagus, muscle, or bodily fluids such as blood, urine, semen, lymphatic fluid, cerebrospinal fluid, or amniotic fluid, or other biological samples, such as tissue culture cells, buccal swabs, mouthwash, stool, tissues slices, and biopsy aspiration. Types of useful DNA samples include blood samples, urine samples, semen samples, lymphatic fluid samples, cerebrospinal fluid samples, amniotic fluid samples, biopsy samples, needle aspiration biopsy samples, cancer samples, tumor samples, tissue samples, cell samples, cell lysate samples, crude cell lysate samples, forensic samples, infection samples, and/or nosocomial infection samples.

Nucleic acid fragments are segments of larger nucleic molecules. Nucleic acid fragments, as used in the disclosed method, generally refer to nucleic acid molecules that have been cleaved. A nucleic acid sample that has been incubated with a nucleic acid cleaving reagent is referred to as a digested sample. A nucleic acid sample that has been digested using a restriction enzyme is referred to as a digested sample.

### E. Kits

The materials described herein as well as other materials can be packaged together in any suitable combination as a kit useful for performing, or aiding in the performance of, the disclosed method. It is useful if the kit components in a given kit are designed and adapted for use together in the disclosed method. For example disclosed are kits for assessing status of a subject, the kit comprising probes for status biomarkers. The kits also can contain status biomarker capture probes, primers for multiple displacement amplification, PCR primers, restriction endonucleases, or a combination.

### F. Mixtures

Disclosed are mixtures formed by performing or preparing to perform the disclosed method. For example, disclosed are mixtures comprising a DNA sample and restriction endonucleases, a DNA sample and primers, a DNA sample and probes, digested, amplified DNA and probes, treated DNA and probes, etc.

Whenever the method involves mixing or bringing into contact compositions or components or reagents, performing the method creates a number of different mixtures. For example, if the method includes 3 mixing steps, after each one of these steps a unique mixture is formed if the steps are performed separately. In addition, a mixture is formed at the completion of all of the steps regardless of how the steps were performed. The present disclosure contemplates these mixtures, obtained by the performance of the disclosed methods as well as mixtures containing any disclosed reagent, composition, or component, for example, disclosed herein.

### G. Systems

Disclosed are systems useful for performing, or aiding in the performance of, the disclosed method. Systems generally comprise combinations of articles of manufacture such as structures, machines, devices, and the like, and compositions, compounds, materials, and the like. Such combinations that are disclosed or that are apparent from the disclosure are contemplated. For example, disclosed and contemplated are systems comprising detection apparatus and arrays of probes.

### H. Data Structures and Computer Control

Disclosed are data structures used in, generated by, or generated from, the disclosed method. Data structures generally are any form of data, information, and/or objects collected, organized, stored, and/or embodied in a composition or medium. A pattern of methylation states and/or levels for status biomarkers stored in electronic form, such as in RAM or on a storage disk, is a type of data structure.

The disclosed method, or any part thereof or preparation therefor, can be controlled, managed, or otherwise assisted by computer control. Such computer control can be accomplished by a computer controlled process or method, can use and/or generate data structures, and can use a computer program. Such computer control, computer controlled processes, data structures, and computer programs are contemplated and should be understood to be disclosed herein.

### Uses

The disclosed methods and compositions are applicable to numerous areas including, but not limited to, assessment of status of cells, tissues, and or subjects, such as by assessment of the presence, stage, risk, etc. of a disease or condition. Other uses include assessing aging and/or general health of cells, tissues, and/or subjects. Other uses are disclosed, apparent from the disclosure, and/or will be understood by those in the art.

### Methods

Disclosed are methods of assessing one or more statuses of a subject. Also disclosed are methods of identifying status biomarkers associated with a status of a subject. Also disclosed are methods of producing status biomarker capture probes.

### A. Method of Using Status Biomarkers

Status biomarkers can be used to assessing one or more statuses of a subject. This can be done by, for example, determining the methylation state of one or more status biomarkers in the subject, and comparing one or more of the determined methylation states to one or more reference methylation states, wherein a difference, lack of a difference, or both in one or more of the determined methylation states and one or more of the reference methylation states indicates one or more statuses of the subject.

i. Determining Methylation State

The methylation state of status biomarkers can be determined using any suitable technique or method. A number of techniques for detecting and determining the presence and level of methylation of DNA are known. Such methods and techniques can be used in the disclosed methods. Generally, methylation can be determined via direct detection of methylated nucleotides or indirectly by altering or separating nucleotides or nucleic acid acids based on the presence or absence of methylation. In some forms, the methylation state can be determined by, for example, treating a DNA sample of the subject to differentiate methylated and unmethylated nucleotides, and detecting the level of methylated forms of the one or more status biomarkers in the treated DNA, detecting the level of unmethylated forms of the one or more status biomarkers in the treated DNA, or both, wherein the level of methylated forms of the status biomarkers, the level of unmethylated forms of the status biomarkers, or both indicates the methylation state of the status biomarkers.

a. Treating DNA Samples

In some forms, treating the DNA sample can be accomplished by, for example, incubating the DNA sample with one or more restriction endonucleases and amplifying the incubated DNA, wherein the restriction endonucleases are methylation-sensitive restriction endonucleases, wherein the level of the status biomarkers in the amplified DNA is lower when the status biomarkers have reduced methylation and the level of the status biomarkers in the amplified DNA is higher when the status biomarkers have increased methylation, wherein the level of the status biomarkers comprise the level of methylated forms of the one or more status biomarkers in the treated DNA, the level of unmethylated forms of the one or more status biomarkers in the treated DNA, or both. An example of such forms of the methods is described in Example 3. A methylation-sensitive restriction endonuclease is a restriction endonuclease that cleaves only at unmethylated recognition and/or cleavage sites. Amplification can distinguish methylated and unmethylated status biomarkers via differential cleavage of restriction endonuclease based on the methylation state of the DNA. For example, cleaving DNA into smaller fragments can reduce the amplification of the DNA. Multiple displacement amplification is useful for this purpose. The methylation state can then be determined by detecting or assessing the presence, absence, or level of amplified nucleic acid.

In some forms, the restriction endonucleases can further comprise at least one methylation-dependent restriction endonuclease. A methylation-dependent restriction endonuclease is a restriction endonuclease that cleaves only at methylated recognition and/or cleavage sites. In some forms, the restriction endonucleases can further comprise at least one methylation-independent restriction endonuclease. A methylation-independent restriction endonuclease is a restriction endonuclease that cleaves at both methylated and unmethylated recognition and/or cleavage sites. In some forms, the restriction endonucleases can comprise AciI and HhaI. In some forms, the restriction endonucleases can comprise McrBC. In some forms, incubating the DNA sample with one or more endonucleases can be accomplished by, for example, incubating different aliquots of the DNA sample with different restriction endonucleases. In some forms, amplifying the incubated DNA can be accomplished by, for example, multiple displacement amplification. An example of such forms of the methods is described in Example 3. Techniques useful for these forms of assessment of methylation states are described in U.S. Patent Application Publication No. 20060292585.

In some forms, treating the DNA sample can be accomplished by, for example, processing the DNA sample with sodium bisulfite. An example of such forms of the methods is described in Example 4. Sodium bisulfite converts cytosine to uridine but does not convert methylcytosine. This allows detection of methylation and methylation levels by detecting cytosine and thymidine. The ratio of cytosine to thymidine can be converted to the relative methylation level.

In some forms, treating the DNA sample can be accomplished by, for example, fragmenting the DNA and separating methylated DNA from unmethylated DNA. An example of such forms of the methods is described in Example 5. In some forms, the DNA can be fragmented by, for example, nebularization, cleavage with a restriction endonuclease, sonication, or a combination. In some forms, methylated DNA can be separated from unmethylated DNA by, for example, binding methylated DNA with a specific binding molecule specific for methyl groups and separating the bound from the unbound DNA. In some forms, the specific binding molecule can comprise, for example, an antibody specific for 5-methyl cytosine, methyl-biding protein MBD1, methyl-biding protein MECP2, or a combination. Numerous techniques and methods for binding and separating molecules are known and can be adapted for use with the disclosed methods to bind and separate methylated form unmethylated DNA.

In some forms, treating the DNA sample can be accomplished by, for example, capturing status biomarker DNA fragments and sequencing the captured status biomarker DNA fragments, wherein the sequencing distinguishes cytosine from methylcytosine, wherein the level of methylcytosine indicates level of methylated forms of the status biomarkers. Examples of such forms of the methods are described in Examples 6 and 7. In some forms, the status biomarker DNA fragments can be captured by, for example, binding DNA fragments in the DNA sample to status biomarker probes attached to a support. In some forms, one or more of the status biomarker probes can specifically hybridize to one or more repetitive DNA sequences, wherein the one or more of the status biomarker probes comprises degenerate sequence representing a consensus sequence for a family of repetitive DNA sequences. In some forms, each of the status biomarker probes can specifically hybridize to one or more repetitive DNA sequences, wherein each of the status biomarker probes comprises degenerate sequence representing a consensus sequence for a family of repetitive DNA sequences. In some forms, the family of repetitive DNA sequences can be a family of repetitive DNA sequences such as the repetitive DNA sequence families listed in, for example, Table 1, Table 12, or Table 13. In some forms, the one or more of the status biomarker probes can comprise different degenerate sequences each representing a consensus sequence for a different one of the families of repetitive DNA sequences such as the repetitive DNA sequence families listed in, for example, Table 1, Table 12, or Table 13. In some forms, the one or more of the status biomarker probes can comprise at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, or 135 different degenerate sequences each representing a different consensus sequence for a different one of the families of repetitive DNA sequences such as the repetitive DNA sequence families listed in, for example, Table 1, Table 12, or Table 13. In some forms, the support can comprise, for example, gel, a bead, a magnetic bead, a plate, a slide, a surface, or a microparticle. In some forms, DNA not captured can be separated from the captured status biomarker DNA fragments. In some forms, the sequencing can be a form of SMRT sequencing.

In some forms, the method can further comprise, after capturing status biomarker DNA fragments and prior to sequencing the captured status biomarker DNA fragments, releasing the captured status biomarker DNA fragments and recapturing the released status biomarker DNA fragments. An example of such forms of the methods is described in Example 7. In some forms, the status biomarker DNA fragments can be recaptured by binding DNA fragments in the DNA sample to secondary status biomarker probes attached to a support. In some forms, one or more of the status biomarker probes can specifically hybridize to one or more repetitive DNA sequences, wherein the one or more of the status biomarker probes comprises degenerate sequence representing a consensus sequence for a family of repetitive DNA sequences. In some forms, each of the status biomarker probes can specifically hybridize to one or more repetitive DNA sequences, wherein each of the status biomarker probes comprises degenerate sequence representing a consensus sequence for a family of repetitive DNA sequences. In some forms, the family of repetitive DNA sequences can be a family of repetitive DNA sequences such as the repetitive DNA sequence families listed in, for example, Table 16 and Table 17. In some forms, the family of repetitive DNA sequences can be a family of repetitive DNA sequences listed in Table 17. For example, the family of repetitive DNA sequences can be the AluY, AluSx, AluSp, AluSg, or AluSc family of repetitive DNA sequences. In some forms, the one or more of the status biomarker probes can comprise different degenerate sequences each representing a consensus sequence for a different one of the families of repetitive DNA sequences such as the repetitive DNA sequence families listed in, for example, Table 16 and Table 17. In some forms, the one or more of the status biomarker probes can comprise different degenerate sequences each representing a consensus sequence for a different one of the families of repetitive DNA sequences listed in Table 16 or 17, such as AluY, AluSx, AluSp, AluSg, or AluSc. In some forms, the support can comprise, for example, gel, a bead, a magnetic bead, a plate, a slide, a surface, or a microparticle. In some forms, DNA not recaptured can be separated from the recaptured status biomarker DNA fragments.

ii. Detecting the Level of Status Biomarkers

In some forms, detecting the level of the status biomarkers can be accomplished via, for example, an array of probes specific for the status biomarkers. An example of such forms of the methods is described in Example 3. This detection is useful for DNA that has been treated to differentially amplify or retain DNA based on the methylation state. In some forms, the array of probes can be, for example, a microarray. Myriad techniques are known for detecting and assessing nucleic acid sequences. Such techniques can be used with the disclosed methods to detect and assess status biomarkers and the status or biomarkers. Multiplex and high throughput techniques are particular useful for this purpose. Thus, for example, the use of arrays and microarrays for detection are particularly useful.

In some forms, detecting the level of the status biomarkers can be accomplished via, for example, amplifying the processed DNA and determining the ratio of cytosine to thymidine in the amplified DNA and converting the ratio to the level of methylated forms of the status biomarkers. An example of such forms of the methods is described in Example 4. This detection is useful for DNA that has been treated with sodium, bisulfite. In some forms, the processed DNA can be amplified via, for example, PCR amplification of the status biomarkers using primers specific for the status biomarkers.

In some forms, detecting the level of the status biomarkers can be accomplished via, for example, PCR amplification of the status biomarkers using primers specific for the status biomarkers. An example of such forms of the methods is described in Example 5. This detection is useful for DNA that has been separated based on methylation of lack of methylation. In some forms, the PCR amplification can be quantitative PCR. In some forms, the PCR amplification can be nanoliter-microarray quantitative PCR.

iii. Analysis of Groups of Status Biomarkers

In some forms, the level of the status biomarkers can be grouped into a plurality of status biomarker families, wherein the level of the status biomarkers in one or more of the families is analyzed, wherein the analyzed level of the status biomarkers in the one or more of the families indicates the methylation state of the status biomarkers in the family. In some forms, the analyzed level of the status biomarkers in one or more of the families can be the average of the levels of the individual status biomarkers in the family. In some forms, one or more of the status biomarker families each independently can consist of, for example, a single class of repetitive DNA element, a single subclass of repetitive DNA element, a single family of repetitive DNA element, a single subfamily of repetitive DNA element, or a combination. In some forms, the analyzed level of the status biomarkers in one or more of the families can be normalized to one or more of the reference methylation states. In some forms, the level of one or more of the status biomarkers can be normalized to one or more of the reference methylation states. In some forms, the level of one or more of the status biomarker families can be normalized to one or more of the reference methylation states. In some forms, the status biomarkers can be grouped according to one or more repetitive DNA sequences that the status biomarkers comprise, wherein each biomarker in each status biomarker family comprises one or more repetitive DNA sequences that belong to a single family of repetitive DNA sequences such as the repetitive DNA sequence families listed in, for example, Table 1, Table 12, or Table 13.

iv. Reference Methylation State

In some forms, one or more of the one or more reference methylation states can be a normal methylation state. In some forms, the normal methylation state can be, for example, the methylation state of a healthy subject, the average of the methylation states of healthy subjects, or the average of the methylation states of a population of subjects. In some forms, one or more of the one or more reference methylation states can be, for example, the methylation state of the same subject at a different time, the methylation state of the same subject at an earlier time, the methylation state of the same subject at a later time, or the methylation state of one or more normal cells, tissues, organs, or a combination of the same subject. In some forms, one or more of the one or more reference methylation states can be the methylation state from non-tumor adjacent tissue. In some forms, one or more of the one or more reference methylation states can be a normal methylation state of a status biomarker family.

v. Determining Genetic State of Status Biomarkers

In some forms, the method can further comprise determining the genetic state of one or more status biomarkers by, for example, comparing one or more of the determined genetic states to one or more reference genetic states, wherein a difference, lack of a difference, or both in one or more of the determined genetic states and one or more of the reference genetic states indicates one or more statuses of the subject. As used herein, “genetic state” refers to a particular sequence or mutation in the biomarker. Thus, for example, a particular SNP in a biomarker is a genetic state of the biomarker. In some forms, determining the genetic state of one or more status biomarkers can be determined in one or more of the DNA samples. The genetic state of biomarkers can be determined using any technique or method that can determine the sequence of a biomarker. Myriad techniques and methods for sequencing and determining the sequence of nucleic acids are known. Such techniques and methods can be used with the disclosed methods.

In some forms, the source of one or more of the DNA samples can be one or more tissues of the subject, organs of the subject, or both. In some forms, the source of one or more of the DNA samples can be a tissue or organ of the subject. In some forms, the source of one or more of the DNA samples can be one or more cells of the subject. In some forms, the source of one or more of the DNA samples can be one or more cells, tissue, skin, lung, head, neck, prostate, breast, ovary, brain, liver, stomach, intestine, kidney, testicle, cervix, uterus, spleen, bone, throat, esophagus, muscle, bodily fluids, blood, urine, semen, lymphatic fluid, cerebrospinal fluid, amniotic fluid, biological samples, tissue culture cells, buccal swabs, mouthwash, stool, tissues slices, biopsy aspiration, or a combination.

vi. Status of Diseases and Conditions Assessed in Subject

In some forms, the subject can be assessed for the status of wellness, level of health, risk to wellness, risk to level of health, or a combination. In some forms, the subject can be assessed for the status of the genome. The status of the genome can be, for example, the level of methylation of status biomarkers in the genome relative to a reference or normal state. A useful reference state for this purpose can be the average methylation state for young subjects and/or healthy subjects. In some forms, the subject can be assessed for the status of aging, risk of aging, or both. In some forms, the subject can be assessed for the status of cancer, risk of cancer, or both. In some forms, the subject can be assessed for the status of stress response. In some forms, the subject can be assessed for the status of diabetes, risk of diabetes, or both. In some forms, the subject can be assessed for the status of heart disease, risk of heart disease, or both. In some forms, the subject can be assessed for the status of genomic instability. In some forms, the subject can be assessed for the status of tumor burden. In some forms, the subject can be assessed for the status of response to treatment. In all of these, changes in methylation state of relevant status biomarkers can indicate the presence or absence of the disease or condition and/or positive or negative changes and/or risks.

vii. Timing and Comparison of Status Assessments

In some forms, the subject can be assessed for a change in one or more statuses. In some forms, the change in one or more of the one or more statuses can be assessed compared to an earlier assessment. In some forms, the earlier assessment can have been made at, for example, an earlier time, prior to diagnosis of a disease or condition, prior to a treatment, following diagnosis of a disease or condition, following treatment, or a combination. In some forms, the change in one or more of the one or more statuses can be assessed following the passage of time, prior to diagnosis of a disease or condition, prior to a treatment, following diagnosis of a disease or condition, following treatment, or a combination. In some forms, assessing the subject can comprise assessing one or more tissues of the subject, organs of the subject, or both. As used herein, assessing a tissue or organ of a subject being assessed for a particular status means that the tissue or organ is assessed for that status and that such assessment of the tissue or organ constitutes the assessment of the subject. In some forms, assessing the subject can comprise assessing a tissue or organ of the subject. In some forms, assessing the subject can comprise assessing one or more cells of the subject.

B. Method of Identifying Status Biomarkers Associated with Diseases and Conditions

Status biomarkers useful for particular states, diseases, and conditions can be identified using the disclosed methods. For example, status biomarkers associated with a status of a subject can be identified by, for example, determining the methylation state of one or more status biomarkers in one or more DNA samples, wherein the DNA samples are from sources that are relevant to one or more specific statuses, and comparing one or more of the determined methylation states to one or more reference methylation states, wherein a difference in one or more of the determined methylation states and one or more of the reference methylation states indicates that the status biomarkers for which the difference in the methylation states is found is a status biomarker associated with one or more of the specific statuses. Particularly useful status biomarkers can be identified by determining the statistical significance of the change in methylation state in the affected sample versus a relevant reference methylation state.

i. Determining Methylation State

In some forms, the methylation state can be determined by, for example, treating a DNA sample of the subject to differentiate methylated and unmethylated nucleotides, and detecting the level of methylated forms of the one or more status biomarkers in the treated DNA, detecting the level of unmethylated forms of the one or more status biomarkers in the treated DNA, or both, wherein the level of methylated forms of the status biomarkers, the level of unmethylated forms of the status biomarkers, or both indicates the methylation state of the status biomarkers.

In some forms, treating the DNA sample can be accomplished by, for example, incubating the DNA sample with one or more restriction endonucleases and amplifying the incubated DNA, wherein the restriction endonucleases are methylation-sensitive restriction endonucleases, wherein the level of the status biomarkers in the amplified DNA is lower when the status biomarkers have reduced methylation and the level of the status biomarkers in the amplified DNA is higher when the status biomarkers have increased methylation, wherein the level of the status biomarkers comprise the level of methylated forms of the one or more status biomarkers in the treated DNA, the level of unmethylated forms of the one or more status biomarkers in the treated DNA, or both. An example of such forms of the methods is described in Example 3.

In some forms, the restriction endonucleases can further comprise at least one methylation-dependent restriction endonuclease. In some forms, the restriction endonucleases can further comprise at least one methylation-independent restriction endonuclease. In some forms, the restriction endonucleases can comprise AciI and HhaI. In some forms, the restriction endonucleases can comprise McrBC. In some forms, incubating the DNA sample with one or more endonucleases can be accomplished by, for example, incubating different aliquots of the DNA sample with different restriction endonucleases. In some forms, amplifying the incubated DNA can be accomplished by, for example, multiple displacement amplification.

In some forms, treating the DNA sample can be accomplished by, for example, processing the DNA sample with sodium bisulfite. An example of such forms of the methods is described in Example 4.

In some forms, treating the DNA sample can be accomplished by, for example, fragmenting the DNA and separating methylated DNA from unmethylated DNA. An example of such forms of the methods is described in Example 5. In some forms, the DNA can be fragmented by, for example, nebularization, cleavage with a restriction endonuclease, sonication, or a combination. In some forms, methylated DNA can be separated from unmethylated DNA by, for example, binding methylated DNA with a specific binding molecule specific for methyl groups and separating the bound form the unbound DNA. In some forms, the specific binding molecule can comprise, for example, an antibody specific for 5-methyl cytosine, methyl-biding protein MBD1, methyl-biding protein MECP2, or a combination.

In some forms, treating the DNA sample can be accomplished by, for example, capturing status biomarker DNA fragments and sequencing the captured status biomarker DNA fragments, wherein the sequencing distinguishes cytosine from methylcytosine, wherein the level of methylcytosine indicates level of methylated forms of the status biomarkers. Examples of such forms of the methods is described in Examples 6 and 7. In some forms, the status biomarker DNA fragments can be captured by, for example, binding DNA fragments in the DNA sample to status biomarker probes attached to a support. In some forms, one or more of the status biomarker probes can specifically hybridize to one or more repetitive DNA sequences, wherein the one or more of the status biomarker probes comprises degenerate sequence representing a consensus sequence for a family of repetitive DNA sequences. In some forms, each of the status biomarker probes can specifically hybridize to one or more repetitive DNA sequences, wherein each of the status biomarker probes comprises degenerate sequence representing a consensus sequence for a family of repetitive DNA sequences. In some forms, the family of repetitive DNA sequences can be a family of repetitive DNA sequences such as the repetitive DNA sequence families listed in, for example, Table 1, Table 12, or Table 13. In some forms, the one or more of the status biomarker probes can comprise different degenerate sequences each representing a consensus sequence for a different one of the families of repetitive DNA sequences such as the repetitive DNA sequence families listed in, for example, Table 1, Table 12, or Table 13. In some forms, the one or more of the status biomarker probes can comprise at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, or 135 different degenerate sequences each representing a different consensus sequence for a different one of the families of repetitive DNA sequences such as the repetitive DNA sequence families listed in, for example, Table 1, Table 12, or Table 13. In some forms, the support can comprise, for example, gel, a bead, a magnetic bead, a plate, a slide, a surface, or a microparticle. In some forms, DNA not captured can be separated from the captured status biomarker DNA fragments. In some forms, the sequencing can be a form of SMRT sequencing.

In some forms, the method can further comprise, after capturing status biomarker DNA fragments and prior to sequencing the captured status biomarker DNA fragments, releasing the captured status biomarker DNA fragments and recapturing the released status biomarker DNA fragments. An example of such forms of the methods is described in Example 7. In some forms, the status biomarker DNA fragments can be recaptured by binding DNA fragments in the DNA sample to secondary status biomarker probes attached to a support. In some forms, one or more of the status biomarker probes can specifically hybridize to one or more repetitive DNA sequences, wherein the one or more of the status biomarker probes comprises degenerate sequence representing a consensus sequence for a family of repetitive DNA sequences. In some forms, each of the status biomarker probes can specifically hybridize to one or more repetitive DNA sequences, wherein each of the status biomarker probes comprises degenerate sequence representing a consensus sequence for a family of repetitive DNA sequences. In some forms, the family of repetitive DNA sequences can be a family of repetitive DNA sequences such as the repetitive DNA sequence families listed in, for example, Table 16 and Table 17. In some forms, the family of repetitive DNA sequences can be a family of repetitive DNA sequences listed in Table 17. For example, the repetitive DNA sequence family can be the AluY, AluSx, AluSp, AluSg, or AluSc family of repetitive DNA sequences. In some forms, the one or more of the status biomarker probes can comprise different degenerate sequences each representing a consensus sequence for a different one of the families of repetitive DNA sequences such as the repetitive DNA sequence families listed in, for example, Table 16 and Table 17. In some forms, the one or more of the status biomarker probes can comprise different degenerate sequences each representing a consensus sequence for a different one of the families of repetitive DNA sequences listed in Table 16 or 17, such as AluY, AluSx, AluSp, AluSg, or AluSc. In some forms, the support can comprise, for example, gel, a bead, a magnetic bead, a plate, a slide, a surface, or a microparticle. In some forms, DNA not recaptured can be separated from the recaptured status biomarker DNA fragments.

ii. Detecting the Level of Status Biomarkers

In some forms, detecting the level of the status biomarkers can be accomplished via, for example, an array of probes specific for the status biomarkers. An example of such forms of the methods is described in Example 3. In some forms, the array of probes can be, for example, a microarray.

In some forms, detecting the level of the status biomarkers can be accomplished via, for example, amplifying the processed DNA and determining the ratio of cytosine to thymidine in the amplified DNA and converting the ratio to the level of methylated forms of the status biomarkers. An example of such forms of the methods is described in Example 4. In some forms, the processed DNA can be amplified via, for example, PCR amplification of the status biomarkers using primers specific for the status biomarkers.

In some forms, detecting the level of the status biomarkers can be accomplished via, for example, PCR amplification of the status biomarkers using primers specific for the status biomarkers. In some forms, the PCR amplification can be quantitative PCR. An example of such forms of the methods is described in Example 5. In some forms, the PCR amplification can be nanoliter-microarray quantitative PCR.

iii. Analysis of Groups of Status Biomarkers

In some forms, the level of the status biomarkers can be grouped into a plurality of status biomarker families, wherein the level of the status biomarkers in one or more of the families is analyzed, wherein the analyzed level of the status biomarkers in the one or more of the families indicates the methylation state of the status biomarkers in the family. In some forms, the analyzed level of the status biomarkers in one or more of the families can be the average of the levels of the individual status biomarkers in the family. In some forms, one or more of the status biomarker families each independently can consist of, for example, a single class of repetitive DNA element, a single subclass of repetitive DNA element, a single family of repetitive DNA element, a single subfamily of repetitive DNA element, or a combination. In some forms, the analyzed level of the status biomarkers in one or more of the families can be normalized to one or more of the reference methylation states. In some forms, the level of one or more of the status biomarkers can be normalized to one or more of the reference methylation states. In some forms, the level of one or more of the status biomarker families can be normalized to one or more of the reference methylation states. In some forms, the status biomarkers can be grouped according to one or more repetitive DNA sequences that the status biomarkers comprise, wherein each biomarker in each status biomarker family comprises one or more repetitive DNA sequences that belong to a single family of repetitive DNA sequences such as the repetitive DNA sequence families listed in, for example, Table 1, Table 12, or Table 13.

iv. Reference Methylation State

In some forms, one or more of the one or more reference methylation states can be a normal methylation state. In some forms, the normal methylation state can be, for example, the methylation state of a healthy subject, the average of the methylation states of healthy subjects, or the average of the methylation states of a population of subjects. In some forms, one or more of the one or more reference methylation states can be, for example, the methylation state of the same subject at a different time, the methylation state of the same subject at an earlier time, the methylation state of the same subject at a later time, or the methylation state of one or more normal cells, tissues, organs, or a combination of the same subject. In some forms, one or more of the one or more reference methylation states can be the methylation state from non-tumor adjacent tissue. In some forms, one or more of the one or more reference methylation states can be a normal methylation state of a status biomarker family.

v. Step of Determining Genetic State of Status Biomarkers

In some forms, the method can further comprise determining the genetic state of one or more status biomarkers by, for example, comparing one or more of the determined genetic states to one or more reference genetic states, wherein a difference, lack of a difference, or both in one or more of the determined genetic states and one or more of the reference genetic states indicates one or more statuses of the subject. In some forms, determining the genetic state of one or more status biomarkers can be determined in one or more of the DNA samples.

In some forms, the source of one or more of the DNA samples can be one or more tissues of the subject, organs of the subject, or both. In some forms, the source of one or more of the DNA samples can be a tissue or organ of the subject. In some forms, the source of one or more of the DNA samples can be one or more cells of the subject. In some forms, the source of one or more of the DNA samples can be one or more cells, tissue, skin, lung, head, neck, prostate, breast, ovary, brain, liver, stomach, intestine, kidney, testicle, cervix, uterus, spleen, bone, throat, esophagus, muscle, bodily fluids, blood, urine, semen, lymphatic fluid, cerebrospinal fluid, amniotic fluid, biological samples, tissue culture cells, buccal swabs, mouthwash, stool, tissues slices, biopsy aspiration, or a combination.

### C. Method of Producing Status Biomarker Probes

The disclosed methods can be used to design and/or produce probes for status biomarkers, including status biomarker capture probes. For example, status biomarker probes can be designed by, for example, selecting a subset of repetitive DNA sequence loci from a set of repetitive DNA sequence loci, and generating a set of status biomarker probe sequences. Status biomarker probes can then be produced by synthesizing one or more status biomarker probes from the status biomarker probe sequences. In some forms, the repetitive DNA sequence loci in the set of repetitive DNA sequence loci can belong to a single one of the families of repetitive DNA sequence such as the repetitive DNA sequence families listed in, for example, Table 1, Table 12, or Table 13, wherein the subset of repetitive DNA sequence loci can be selected by identifying those repetitive DNA sequence loci that comprise a repetitive DNA sequence belonging to one of the families of repetitive DNA sequences such as the repetitive DNA sequence families listed in, for example, Table 16 and Table 17.

In some forms, each status biomarker probe sequence in the set can have a length of, for example, 15, 20, 25, 30, 35, 40, 45, 50, 55, 60, 65, 70, 75, 80, 85, 90, 95, or 100 bases or more, wherein each status biomarker probe represented in the set of status biomarker probe sequences can hybridize to, for example, at least 1%, 2%, 3%, 4%, 5%, 6%, 7%, 8%, 9%, 10%, 11%, 12%, 13%, 14%, 15% of the repetitive DNA sequence loci in the selected subset of repetitive DNA sequence loci. In some forms, each status biomarker probe can have the sequence of one of the status biomarker probe sequences.

In some forms, the repetitive DNA sequence loci in the set of repetitive DNA sequence loci can belong to a single one of the families of repetitive DNA sequence LTR54B, MER11B, MER34B, LTR56, THE1B, HERV9, LTR14C, HERVFH21, LTR6B, LTR46, MLT1D, MER67D, HERVK11, LTR10B, HERVK22, MER6, MER66C, MLT1G1, MER4D, and MLTD2. In some forms, the repetitive DNA sequence in the subset of repetitive DNA sequence loci can belong to one of the families of repetitive DNA sequences listed in Table 16 or 17, such as AluY, AluSx, AluSp, AluSg, AluSc, LTR9, or LTR9B.

In some forms, the method can further comprise selecting one or more additional subsets of repetitive DNA sequence loci each from a different additional set of repetitive DNA sequence loci, generating one or more additional sets of status biomarker probe sequences each based on one of the one or more additional subsets, and synthesizing one or more additional status biomarker probes, wherein each additional status biomarker probe has the sequence of one of the additional status biomarker probe sequences. In some forms, the repetitive DNA sequence loci in each additional set of repetitive DNA sequence loci can independently belong to a different single one of the families of repetitive DNA sequence such as the repetitive DNA sequence families listed in, for example, Table 1, Table 12, or Table 13, wherein the repetitive DNA sequence loci in the set of repetitive DNA sequence loci and in each additional set of repetitive DNA sequence loci belong to different families of repetitive DNA sequence.

In some forms, the repetitive DNA sequence loci in the each additional set of repetitive DNA sequence loci can independently belong to a single one of the families of repetitive DNA sequence LTR54B, MER11B, MER34B, LTR56, THE1B, HERV9, LTR14C, HERVFH21, LTR6B, LTR46, MLT1D, MER67D, HERVK11, LTR10B, HERVK22, MER6, MER66C, MLT1G1, MER4D, and MLTD2. In some forms, each status biomarker probe sequence in the set can have a length of, for example, 15, 20, 25, 30, 35, 40, 45, 50, 55, 60, 65, 70, 75, 80, 85, 90, 95, or 100 bases or more. In some forms, each status biomarker probe represented in the set of status biomarker probe sequences can hybridize to, for example, at least 1%, 2%, 3%, 4%, 5%, 6%, 7%, 8%, 9%, 10%, 11%, 12%, 13%, 14%, 15% of the repetitive DNA sequence loci in the selected subset of repetitive DNA sequence loci. In some forms, the set of status biomarker probe sequences can comprise from 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 32, 34, 35, 36, 38, 40, 42, 44, 45, 46, 48, 50, 55, 60, 65, 70, 75, 80, 85, 90, 95, 100, 110, 120, 130, 140, 150, 160, 170, 180, 190, or 200 to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 32, 34, 35, 36, 38, 40, 42, 44, 45, 46, 48, 50, 55, 60, 65, 70, 75, 80, 85, 90, 95, 100, 110, 120, 130, 140, 150, 160, 170, 180, 190, or 200 status biomarker probe sequences. In some forms, the set of status biomarker probe sequences can comprise from 5 to 100 status biomarker probe sequences. In some forms, the set of status biomarker probe sequences can comprise from 10 to 100 status biomarker probe sequences. In some forms, one or more of the additional sets of status biomarker probe sequences each can comprise from 1 to 100 status biomarker probe sequences. In some forms, the one or more additional sets of status biomarker probe sequences each can comprise from 5 to 100 status biomarker probe sequences. In some forms, the one or more additional sets of status biomarker probe sequences each can comprise from 10 to 100 status biomarker probe sequences.

Status biomarker probes can be designed and produce for any desired status biomarker or family of status biomarkers. For example, capture probes for preferred status biomarkers can be designed by:

a. obtaining the RepeatMasker annotation for all members of a preferred status biomarker family, said annotation comprising the genomic coordinates of each member of the chosen repetitive element family, as well as the annotated DNA sequence;

b. re-organizing the DNA sequences in the list so that all are in the 5′ to 3′ orientation;

c. examining each candidate status biomarker locus by defining a window of 1000 bases, centered in the middle of the repetitive element sequence, and then performing a query of the RepeatMasker annotation to find any other repeats present in the window, whereby those co-localized or neighbor repetitive elements belong to a list of preferred neighbor families (such as those listed in Table 16 and Table 17);

d. choosing the subset of the coordinates corresponding to repetitive elements satisfying the criteria that they contain a neighbor present in the list of neighbors; this is the preferred candidate coordinate and sequence list;

e. generating a list, using standard computational tools, of between 1 and 100 oligonucleotides each with a length of 100 bases or more, each probe capable of forming duplex structures with more than 5% of the sequences present in the preferred candidate sequence list; the duplex structures can contain several mismatches, as long as they are deemed capable of forming a duplex stable enough for performing sequence capture (design criteria for such capture probes are published and well known in the art).

The designed capture probes can be produced and used by, for example, performing synthesis (as DNA or RNA) of the designed oligonucleotides (between 1 and 100 different sequences), and utilizing these oligonucleotides, as a mixture in solution, or as a collection of probes bound on a microarray surface, for capturing fragmented genomic DNA from a biological sample, using methods well know in the published art.

### D. Statistical Analysis

1. Random Forest

Random forest (Breiman 2001) is a classifier that is consisted of many decision trees. The following is the procedure of constructing an individual decision tree. Suppose there are n observations and p variables (or features) in the data set. (1) Randomly draw a bootstrap sample of size n with replacement from the data set. This set is called the training set and is used to construct a decision tree. (2) A pre-specified fixed number of variables, say m, is drawn at random from the p variables. The parameter m is chosen such that it is much smaller than p. (3) A tree is constructed from the top down. At each node, the variable that yields the best split is chosen to split the node. (4) Repeat step 3 to grow the tree until no split can further improve the classification. No pruning is conducted.

To classify a new case, run it through all trees in the forest. Each tree gives a classification, or called a “vote”. And the final classification given by the forest takes the majority votes of all trees. To obtain an estimate of error rates, the set of observations that are not sampled in each tree, which is called the out-of-bag (OOB) set and is about one third of the original data, is used for cross validation. More specifically, for each OOB case, run it down the decision tree and obtain a classification or a vote. At the end of all runs, each case has the final classification by simple majority OOB votes. This gives an estimate of the error rates.

The advantages of random forest include: excellent classification accuracy; fast computation speed; efficient handling of large data sets; providing proximities between pairs of cases; generating importance measures for all variables; no need of extra test sets.

2. Support Vector Machine

In Support Vector Machine (SVM) (Vapnik 1998) a set of features that describes an observation is called a vector. SVM classifies observations by construct hyperplanes that optimally separate the data into different classes, i.e., vectors of different classes are on different sides of the hyperplanes. The vectors close to the hyperplanes are called support vectors. The goal of SVM is to find optimal hyperplanes by maximizing the distances between the support vectors and the hyperplanes. SVM is computationally efficient and can handle large data sets.

Support Vector Machine-Recursive Feature Elimination (SVM-RFE) (Guyon et al., 2002) selects features in a sequential backward elimination manner, which starts with all the features and discards one feature at a time.

3. Others

Several statistical analyses can be performed. A list of other analyses includes, but is not limited to, Linear discriminant analysis (McLachlan 2004), Logistic regression (Agresti 2002), Classification and Regression Trees (CART) (Breiman 1984), Neural Networks (Marques de Sa 2001), Bayesian Additive Regression Trees (Chipman 2006).

### A. Detection

Any analyte, including the various compounds and compositions disclosed herein, can be detected. For example, status biomarkers, repetitive DNA sequence, repetitive DNA sequence loci, families of status biomarkers, families of repetitive DNA sequences, etc. can be detected. Detection of status biomarkers can be by, for example, detecting the level, amount, presence, or a combination, of the analyte in a sample or assay. As described below and elsewhere herein, the manner of detection of status biomarkers can be based on the treatment of DNA samples and generally can be in service of detecting and determining the methylation state and presence of methylation in status biomarkers. Detection of the disclosed compounds and compositions can be accomplished in any of a variety of ways and using any of a variety of techniques. Many such detection techniques are known and can be readily adapted for use in the disclosed methods. In most cases, the disclosed methods do not depend on particular techniques of detection. However, certain techniques and reagents are useful for detecting different types of compounds and compositions. Those of skill in the art are aware of the selection of particular techniques for the detection of particular compounds and compositions. Detection can, but need not, involve an element of quantitation.

Detection can be of a class of compounds or compositions or of specific compounds or compositions. Although the disclosed methods generally involve detection of specific compounds and compositions, such as specific DNA molecules, the disclosed methods can also be used to detect classes or groups of compounds or compositions, generally via one or more common properties. In other forms, multiple different specific compounds and/or compositions can be detected. Such detection accomplished in the same assay or run (or in separate assays of runs performed at the same time), can generally be referred to as multiplex detection.

Detection can involve or include, for example, measuring, sequencing, identification, or a combination. Measurement is useful for determining abundances and levels of an analyte in a sample. Sequencing is useful for identifying nucleic acid sequence and molecules. Uses and forms of detection in the context of the disclosed methods are also described elsewhere herein.

Detection can involve a variety of forms. For example, detecting one or more of the status biomarkers can be accomplished using a probe corresponding to a unique sequence in the status biomarker.

1. Measuring

Any analyte, including the various compounds and compositions disclosed herein, can be detected by measuring, for example, the level, amount, presence, or a combination, of the analyte in a sample or assay. For example, the methylation state and/or level of status biomarkers, repetitive DNA sequence, repetitive DNA sequence loci, families of status biomarkers, families of repetitive DNA sequences, etc. can be measured. Measurement of the level, amount, presence, or a combination, of the analyte can also be accomplished when detection is not an explicit object. Similar to detection, measurement of the disclosed compounds and compositions can be accomplished in any of a variety of ways and using any of a variety of techniques. Many such measurement techniques are known and can be readily adapted for use in the disclosed methods. In most cases, the disclosed methods do not depend on particular techniques of measurement. Measurement can involve an element of quantitation. Many techniques are known for measuring abundances and levels of an analyte in a sample. Such techniques can be adapted for use with the disclosed methods.

2. Sequencing

Nucleic acid sequences and molecules can be detected, measured, identified, and so on, via sequencing. In the context of nucleic acid sequences and molecules, sequencing refers to the determination or identification of some or all of the nucleotide base sequence of a nucleic acid sequence or molecule. Numerous techniques for nucleic acid sequencing are known and can be used with the disclosed methods. Examples of useful types of sequencing techniques include techniques involving detection of individual nucleotide bases (such as by detection of terminated primer extension products) and detection of multiple nucleotide bases (such as by hybridization of probes of known sequence). Any suitable sequencing technique can be used with the disclosed methods. Sequencing is particularly useful for identifying nucleic acid sequences and molecules.

Particularly useful sequencing techniques are those that can generate large amounts of sequence data quickly and accurately. High-throughput and ultra-high throughput sequencing provides a number of advantages, the main two being faster results and the ability to detect and measure a large number of nucleic acid molecules. Examples of useful high-throughput sequencing techniques include Solexa™ sequencing, SOLiD™ sequencing, and sequencing using a Illumina Genome Analyzer™ or a 454™.

Illumina Sequencing technology is based on massively parallel sequencing of millions of fragments using reversible terminator-based sequencing chemistry. Illumina

Sequencing technology relies on the attachment of randomly fragmented genomic DNA to a planar, optically transparent surface. Attached DNA fragments are extended and bridge amplified to create an ultra-high density sequencing flow cell with hundreds of millions of clusters, each containing ˜1,000 copies of the same template. These templates are sequenced using a four-color DNA sequencing-by-synthesis technology that employs reversible terminators with removable fluorescent dyes. This allows high accuracy and true base-by-base sequencing, eliminating sequence-context specific errors and enabling sequencing through homopolymers and repetitive sequences. High-sensitivity fluorescence detection is achieved using laser excitation and total internal reflection optics. Sequence reads are aligned against a reference genome and genetic differences are called using specially developed data analysis pipeline software.

The SOLiD System involves depositing beads containing template DNA fragments to be sequenced onto a glass slide. Primers hybridize to a sequence within the template. A set of four fluorescently labeled di-base probes compete for ligation to the sequencing primer. Specificity of the di-base probe is achieved by interrogating every 1st and 2nd base in each ligation reaction. Multiple cycles of ligation, detection and cleavage are performed with the number of cycles determining the eventual read length. Following a series of ligation cycles, the extension product is removed and the template is reset with a primer complementary to the n−1 position for a second round of ligation cycles. Five rounds of primer reset are completed for each sequence tag. Through the primer reset process, each base is interrogated in two independent ligation reactions by two different primers. For example, the base at read position 5 is assayed by primer number 2 in ligation cycle 2 and by primer number 3 in ligation cycle 1. This dual interrogation is fundamental to the unmatched accuracy characterized by the SOLiD System.

The SOLiD System relies on open slide format and flexible bead densities to enable increases in throughput with protocol and chemistry optimizations. The SOLiD System provides system accuracy greater than 99.94%, due to 2 base encoding. 2 Base encoding enables unique error checking capability, providing higher confidence in each call. The SOLiD™ System can generate over 20 gigabases and 400M tags per run. The independent flow cell configuration of the SOLID Analyzer two completely independent experiments in a single run. The combination of multiple slide configuration and sample multiplexing capability enables you to analyze multiple samples cost effectively for a variety of applications. The SOLiD System supports sample preparation for mate-paired libraries with insert sizes ranging from 600 bp up to 10 kbp. This broad range of insert sizes combined with ultra high throughput and flexible 2 flow cell configuration enables more precise characterization of structural variation across the genome.

3. Identification

In the context of the disclosed methods, identification refers to determination of the particular type or instance of a thing, such as of the disclosed status biomarkers, repetitive DNA sequence, repetitive DNA sequence loci, families of status biomarkers, families of repetitive DNA sequences, etc. Thus, for example, a status biomarker can be identified by determining part of its sequence, where the sequence is characteristic of that status biomarker. In the disclosed method, a number of components are, or can be designed, to correspond to, be complementary to, or be for particular other components. By such correspondence, identification of one component can often allow identification of any other components that correspond. For example, a probe can be designed with a target complement sequence that is complementary to a particular sequence of a status biomarker of interest. The probe can be said to correspond to, or to be for, the status biomarker of interest. When used in the disclosed methods, detection or identification of the probe can result in the detection of the presence, or identification, of the corresponding status biomarker in the sample.

### DEFINITIONS

The term “hit” refers to a test compound that shows desired properties in an assay. The term “test compound” refers to a chemical to be tested by one or more screening method(s) as a putative modulator. A test compound can be any chemical, such as an inorganic chemical, an organic chemical, a protein, a peptide, a carbohydrate, a lipid, or a combination thereof. Usually, various predetermined concentrations of test compounds are used for screening, such as 0.01 micromolar, 1 micromolar and 10 micromolar. Test compound controls can include the measurement of a signal in the absence of the test compound or comparison to a compound known to modulate the target.

The terms “higher,” “increases,” “elevates,” or “elevation” refer to increases above basal levels, e.g., as compared to a control. The terms “low,” “lower,” “reduces,” or “reduction” refer to decreases below basal levels, e.g., as compared to a control.

The term “modulate” as used herein refers to the ability of a compound to change an activity in some measurable way as compared to an appropriate control. As a result of the presence of compounds in the assays, activities can increase or decrease as compared to controls in the absence of these compounds. Preferably, an increase in activity is at least 25%, more preferably at least 50%, most preferably at least 100% compared to the level of activity in the absence of the compound. Similarly, a decrease in activity is preferably at least 25%, more preferably at least 50%, most preferably at least 100% compared to the level of activity in the absence of the compound. A compound that increases a known activity is an “agonist”. One that decreases, or prevents, a known activity is an “antagonist.”

The term “inhibit” means to reduce or decrease in activity or expression. This can be a complete inhibition or activity or expression, or a partial inhibition. Inhibition can be compared to a control or to a standard level. Inhibition can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%.

The term “monitoring” as used herein refers to any method in the art by which an activity can be measured.

The term “providing” as used herein refers to any means of adding a compound or molecule to something known in the art. Examples of providing can include the use of pipettes, pipettemen, syringes, needles, tubing, guns, etc. This can be manual or automated. It can include transfection by any mean or any other means of providing nucleic acids to dishes, cells, tissue, cell-free systems and can be in vitro or in vivo.

The term “preventing” as used herein refers to administering a compound prior to the onset of clinical symptoms of a disease or conditions so as to prevent a physical manifestation of aberrations associated with the disease or condition.

The term “in need of treatment” as used herein refers to a judgment made by a caregiver (e.g. physician, nurse, nurse practitioner, or individual in the case of humans; veterinarian in the case of animals, including non-human mammals) that a subject requires or will benefit from treatment. This judgment is made based on a variety of factors that are in the realm of a care giver's expertise, but that include the knowledge that the subject is ill, or will be ill, as the result of a condition that is treatable by the disclosed compounds.

As used herein, “subject” includes, but is not limited to, animals, plants, bacteria, viruses, parasites and any other organism or entity. The subject can be a vertebrate, more specifically a mammal (e.g., a human, horse, pig, rabbit, dog, sheep, goat, non-human primate, cow, cat, guinea pig or rodent), a fish, a bird or a reptile or an amphibian. The subject can be an invertebrate, more specifically an arthropod (e.g., insects and crustaceans). The term does not denote a particular age or sex. Thus, adult and newborn subjects, as well as fetuses, whether male or female, are intended to be covered. A patient refers to a subject afflicted with a disease or disorder. The term “patient” includes human and veterinary subjects.

By “treatment” and “treating” is meant the medical management of a subject with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. It is understood that treatment, while intended to cure, ameliorate, stabilize, or prevent a disease, pathological condition, or disorder, need not actually result in the cure, ameliorization, stabilization or prevention. The effects of treatment can be measured or assessed as described herein and as known in the art as is suitable for the disease, pathological condition, or disorder involved. Such measurements and assessments can be made in qualitative and/or quantitative terms. Thus, for example, characteristics or features of a disease, pathological condition, or disorder and/or symptoms of a disease, pathological condition, or disorder can be reduced to any effect or to any amount.

A cell can be in vitro. Alternatively, a cell can be in vivo and can be found in a subject. A “cell” can be a cell from any organism including, but not limited to, a bacterium.

By the term “effective amount” of a compound as provided herein is meant a nontoxic but sufficient amount of the compound to provide the desired result. As will be pointed out below, the exact amount required will vary from subject to subject, depending on the species, age, and general condition of the subject, the severity of the disease that is being treated, the particular compound used, its mode of administration, and the like. Thus, it is not possible to specify an exact “effective amount.” However, an appropriate effective amount can be determined by one of ordinary skill in the art using only routine experimentation.

By “pharmaceutically acceptable” is meant a material that is not biologically or otherwise undesirable, i.e., the material can be administered to an individual along with the selected compound without causing any undesirable biological effects or interacting in a deleterious manner with any of the other components of the pharmaceutical composition in which it is contained.

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a status biomarker” includes a plurality of such status biomarkers, reference to “the status biomarker” is a reference to one or more status biomarkers and equivalents thereof known to those skilled in the art, and so forth.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps.

Those skilled in the art will recognize, or be able to ascertain using no more than routine experimentation, many equivalents to the specific embodiments of the method and compositions described herein. Such equivalents are intended to be encompassed by the following claims.

### EXAMPLES

### A. Example 1

### Combined Repetitive DNA Biomarkers for Measuring Genomic Instability, Stress, Aging, Cancer Risk, Tumor Burden, and Response to Therapy

1. Introduction

There is a need for sensitive and accurate assays capable of detecting the presence of abnormal cells in tissues. Such abnormal cells may represent in aging tissues, dysplasia, carcinoma in situ, cancer, or metastatic cancer. There is also a need for accurate assays capable of reporting the response of patients to therapies whose purpose is to kill tumor cells, or to reduce the number of abnormal or dysplastic cells in an organ compartment.

The detection of circulating DNA derived from dead or damaged cells is an attractive strategy for implementing assays that are useful for the aforementioned purposes. DNA is a very stable molecule, and can persist for a long time in the circulation. Thus, when tumor cells or other abnormal cells die, the DNA may be detected in the circulation. There is a large literature reporting the detection in the circulating of DNA derived from tumors, or from abnormal cells. Recently Sunami et al (2008) reported the quantification of LINE-1 in circulating DNA as a molecular biomarker of breast cancer. An earlier report by Rago et al. (2007) had reported the assessment of human tumor burdens in mouse xenografts by the analysis of circulating human-specific LINE-1 DNA. In the mouse study, a spike in the amount of human LINE-1 DNA present in plasma was shown to increase after tumor cytotoxic therapy, demonstrating the utility of this biomarker for monitoring drug responses. In both of these studies the detection of circulating DNA is facilitated by the fact that each dying cell releases thousands of molecules of LINE-1 DNA, and therefore the limit of detection of these assays corresponds to a relatively small number of cells of interest.

Recently Korshunova et al (2008) reported the results of a comprehensive methylation pattern analysis from breast cancer clinical tissues and sera obtained using massively parallel bisulphite pyrosequencing of four different gene loci in the human genome. The detailed sequencing analysis of more than 700,000 DNA fragments derived from more than 50 individuals (cancer and cancer-free) revealed an unappreciated complexity of genomic cytosine-methylation patterns in both tissue derived and circulating DNAs. Key observations of this study were as follows: First, there were no tumor-specific molecular methylation sequence patterns obtained from any of the four tested loci. Tumors and cancer-free tissues as well as sera of cancer-free individuals contained nearly every conceivable cytosine methylation pattern. A great variety of methylated molecules were present in all samples, yet no special type of methylation pattern could be found in a statistically meaningful way exclusively in cancerous or exclusively in normal tissues (or serum). At all four tested loci, while there were no tumor-specific molecules, there were different tumor-specific loads of abnormally methylated DNA molecules. The second important finding of the work was that the levels of methylation vary greatly among tumor samples, but yet, little variation in methylation levels was found in samples considered histologically normal. The third important observation of this study involved the quantification of the background level of circulating, abnormally methylated molecules in cancer-free patient sera. According to cancer-specific mutation-based estimations, tumor DNA in serum at early stages of disease is present at a relative abundance of about 12 haploid genomes for every 10,000 somatically normal haploid genomes (0.12%) or less. Expected methylation signals from such minute amounts are so close to the level of background (in most cases around several tens of the percent) that robust detection of tumor-shed DNA was problematic, especially in the case of an epigenetically complex background.

Over time, many laboratories have been identifying, and will continue to identify biomarkers that are indicative of aging, dysplasia, or cancer. A common property of the majority of these cancer markers is that they vary depending on the tissue of origin. For example, Ince et al. (2007) have published findings that indicate that transformation of different breast human breast epithelial cell types leads to distinct tumor phenotypes. This is the case because tumor phenotype tends to resemble progenitor tissue due to natural lineage differentiation relationships. The present disclosure provides methods of identifying biomarkers that can be of general utility in detecting all types of tumors, such that they will serve for detection of any tumor type, as well as for the detection of dysplasia in all types of tissues.

Changes in DNA methylation patterns of non-coding genomic compartments of cancer cells have been explored. The analysis of repetitive DNA in cancers of the head and neck were focused on. Kurshunova et al. (2008) did not examine the methylation abnormalities of repetitive DNA. Most reported studies on DNA methylation of repetitive elements have measured the methylation level of repetitive elements by obtaining average metrics, representative of mixtures of thousands of different repetitive elements. A novel method that reports the DNA methylation level of each individual locus harboring a repetitive DNA element was utilized. The method used for this analysis provided a convenient tool to survey the methylation status of individual repetitive elements.

2. Results

i. Methylation Patterns of Major Classes and Families of DNA Repeats

The DNA methylation profiles of 33 tumors and 17 non-tumor adjacent tissue samples obtained from patients with head and neck squamous carcinoma (HNSCC) were analyzed. DNA methylation profiles from the buccal epithelia of 10 normal individuals were also generated, which served as controls. A novel microarray method for analysis of DNA methylation, based on the use of methylation sensitive as well as methylation dependent endonucleases, enables the interrogation of methylation levels in all compartments of the genome, including repetitive elements. Analysis of a substantial set of samples of squamous carcinomas of the head and neck, as well as non-tumor adjacent tissue and normal controls, reveals a complex framework of epigenetic dysregulation, where loss of methylation differentially affect distinct families of repetitive elements. Predominantly the younger, primate-specific members of retroelement families suffer the most dramatic loss of methylation, with the exception of some extremely young, human-specific retroelements. These complex patterns of differential susceptibility to disruption of silencing are probably a result of the natural history of evolutionary domestication of retroelements in genomes, in interplay with a minimal time requirement for strong silencing to be established. Primate-specific subfamilies of LINE-1 elements appear to suffer a particularly pronounced loss of methylation in tumors, with the most dramatic changes apparently observed for those primate retroelements with conserved promoter regions and longer sequences.

ii. Repetitive Elements as Cancer Biomarkers.

DNA methylation status of repetitive elements has been used as biomarkers for cancer risk. The majority of these studies have focused on the DNA methylation status of Line-1 elements, while a few have utilized Alu elements instead. A sampling of seven exemplary publications on this subject was examined, and five pairs of different DNA primer sequences were identified that have been utilized to amplify Line-1 DNA sequences, typically after treatment of the DNA with sodium bisulfite. Using the computer program FASTA, the positions in the human genome where these five different sets of primers are perfectly aligned were identified, and predicted the exact composition of the amplified repetitive elements, from the standpoint of repeat masker annotation. The sequences that are predicted to be amplified by the polymerase chain reaction in every case represent a complex mixture of Line-1 elements corresponding to different families of different evolutionary age. The lineages that are most highly represented, shown in the table, are L1 HS (human specific) and L1PA2, a relatively recent lineage that originated in simians approximately 7.6 millions years ago (see Table 2).

TABLE 2
A compilation of observations about ages of lineages
within families of repetitive elements

| | | Estimated age of | |
|---|---|---|---|
| Element | Element | appearance in the | |
| Family | subfamily | genome (MYA) | references |
| L1PA | L1PA1 | 3.1 | Table 1 in Khan et al. |
| | | | 2006 |
| L1PA | L1PA2 | 7.6 | Table 1 in Khan et al. |
| | | | 2006 |
| L1PA | L1PA3 | 12.5 | Table 1 in Khan et al. |
| | | | 2006 |
| L1PA | L1PA4 | 18.0 | Table 1 in Khan et al. |
| | | | 2006 |
| L1PA | L1PA5 | 20.4 | Table 1 in Khan et al. |
| | | | 2006 |
| L1PA | L1PA6 | 26.8 | Table 1 in Khan et al. |
| | | | 2006 |
| L1PA | L1PA7 | 31.4 | Table 1 in Khan et al. |
| | | | 2006 |
| L1PA | L1PA8 | 40.9 | Table 1 in Khan et al. |
| | | | 2006 |
| L1PA | L1PA8A | 41.7 | Table 1 in Khan et al. |
| | | | 2006 |
| L1PA | L1PA10 | 46.4 | Table 1 in Khan et al. |
| | | | 2006 |
| L1PA | L1PA11 | 53.3 | Table 1 in Khan et al. |
| | | | 2006 |
| L1PA | L1PA13B | 59.8 | Table 1 in Khan et al. |
| | | | 2006 |
| L1PA | L1PA12 | 59.7 | Table 1 in Khan et al. |
| | | | 2006 |
| L1PA | L1PA13A | 59.9 | Table 1 in Khan et al. |
| | | | 2006 |
| L1PA | L1PA14 | 60.6 | Table 1 in Khan et al. |
| | | | 2006 |
| L1PA | L1PA15 | 70.5 | Table 1 in Khan et al. |
| | | | 2006 |
| L1PA | L1PA16 | 79.7 | Table 1 in Khan et al. |
| | | | 2006 |
| L1PA | L1PA17 | 101.1 | Table 1 in Khan et al. |
| | | | 2006 |
| L1PB | L1PB1 | 46.7 | Table 1 in Khan et al. |
| | | | 2006 |
| L1PB | L1PB2 | 58.2 | Table 1 in Khan et al. |
| | | | 2006 |
| L1PB | L1PB3 | 73.5 | Table 1 in Khan et al. |
| | | | 2006 |
| L1PB | L1PB4 | 96.0 | Table 1 in Khan et al. |
| | | | 2006 |
| L1MA | L1MA1 | 61.6 | Table 1 in Khan et al. |
| | | | 2006 |
| L1MA | L1MA2 | 65.8 | Table 1 in Khan et al. |
| | | | 2006 |
| L1MA | L1MA3 | 68.1 | Table 1 in Khan et al. |
| | | | 2006 |
| L1MA | L1MA4 | 102.3 | Table 1 in Khan et al. |
| | | | 2006 |
| L1MA | L1MA5 | 98.6 | Table 1 in Khan et al. |
| | | | 2006 |

In some cases, L1PA3 elements are also highly represented. In conclusion, primers used in the published for the amplification of Line-1 biomarkers are not designed optimally, and do not sample specifically any chosen L1 subfamily, but rather a mixture of subfamilies. A consequence of the sub-optimal design of all of the primer-pairs reported in the literature is that the Line-1 sequences being sampled to generate DNA methylation metrics are not those genomic sequences that contain the most useful information related to the onset of dysplasia and cancer.

iii. Generation of Subsets of Genomic Repetitive Element Sequences that are Useful for Distinguishing Tumors from Adjacent Nontumor Tissue, and from Healthy Normal Tissue

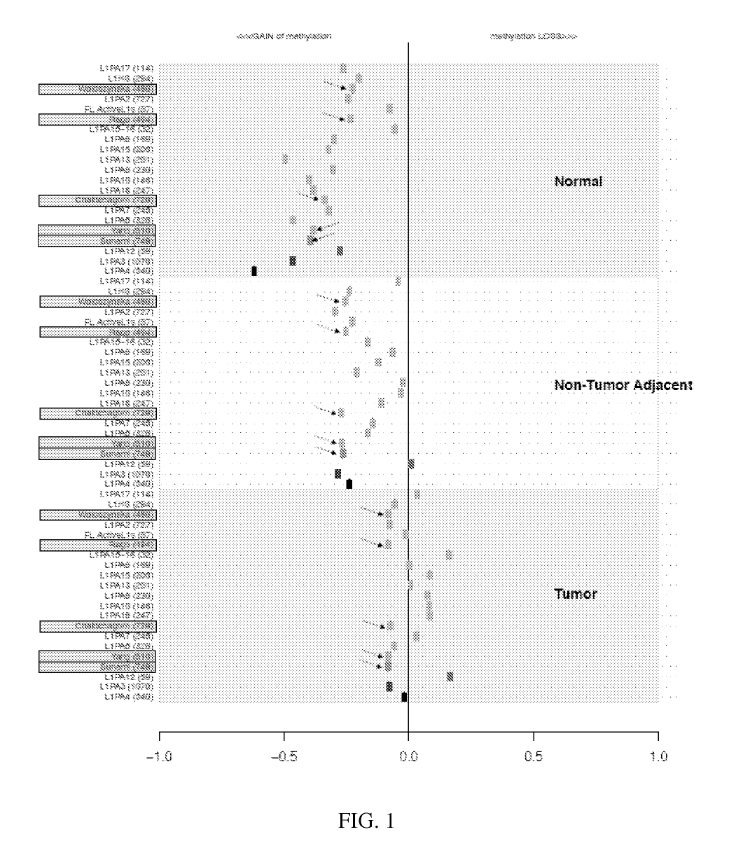

A list of DNA methylation values calculated as the average methylation of each category or sub-category of repetitive element was generated, including reprotransposon-derived elements, and DNA-transposon-derive elements. The values are obtained for individual experiments, and each average is generated my multiple probes of the same category, where each category will comprise anywhere from 20 to 48,000 probes. The data for individual members of each individual family was then analyzed. As an example of this analysis, the plot shown in FIG. 1 represents the DNA methylation levels for subclasses of Line1 elements present in mammals. The order the sub-categories is constant in all three of the subsections in the plot. It was established based on the extent of variation in the plotted distributions using Shannon entropy information content metric. Only Normal and Tumor experiments were used to calculate the Shannon's Information metric.

The arrows point to DNA methylation values calculated by taking the fractional values obtained from Table 1, and calculating a weighed average that takes into account the fractional composition, as well as the DNA methylation value of each class represented in the mixture. The “in-silico PCR” values represent the simulated prediction of the DNA methylation metrics that would be obtained if one were to perform a PCR experiment based on the use of published primer sequences, and utilizing as biological material the DNA obtained from the samples of cancer of the head and neck. It is notable that none of the DNA methylation values indicated by the arrow represents metrics with optimal information content.

Among the publications cited as examples of papers teaching the use of Line-1 primers as cancer biomarkers, the most recent (Choi et al, 2009, using Line-1 primers designed by Woloszynska-Read et al., 2008) point out the observation that “5-mdC level in leukocyte DNA was significantly lower in breast cancer cases than healthy controls (p=0.001), but no significant case-control differences were observed with LINE-1 methylation”. It is not surprising that Choi et al. did not observe significant case-control differences, in the light of the data presented herein (see FIG. 1) that the Woloszynska-Read primer set generates PCR amplicons with relatively low information contend with regards to dysplasia or cancer. The data presented herein show the subfamilies with the largest information content are L1PA4, L1PA3, and L1PA12, none of which have been reported in the literature as candidate cancer biomarkers.

iv. Statistical Analysis of Optimal Repetitive DNA Biomarkers Selected from a Large Set of Repetitive Elements that Suffer DNA Methylation Changes in Dysplasia and Cancer.

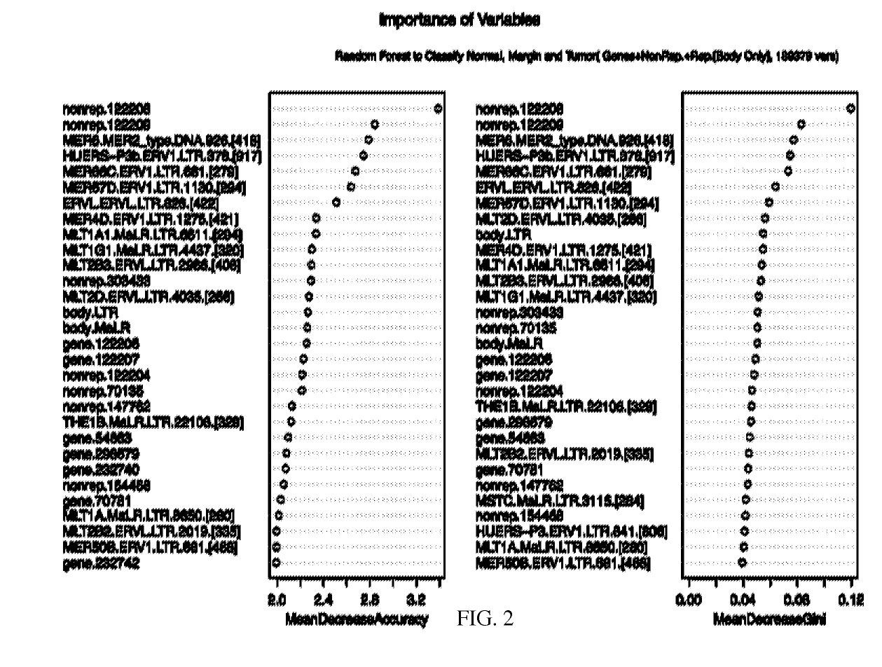

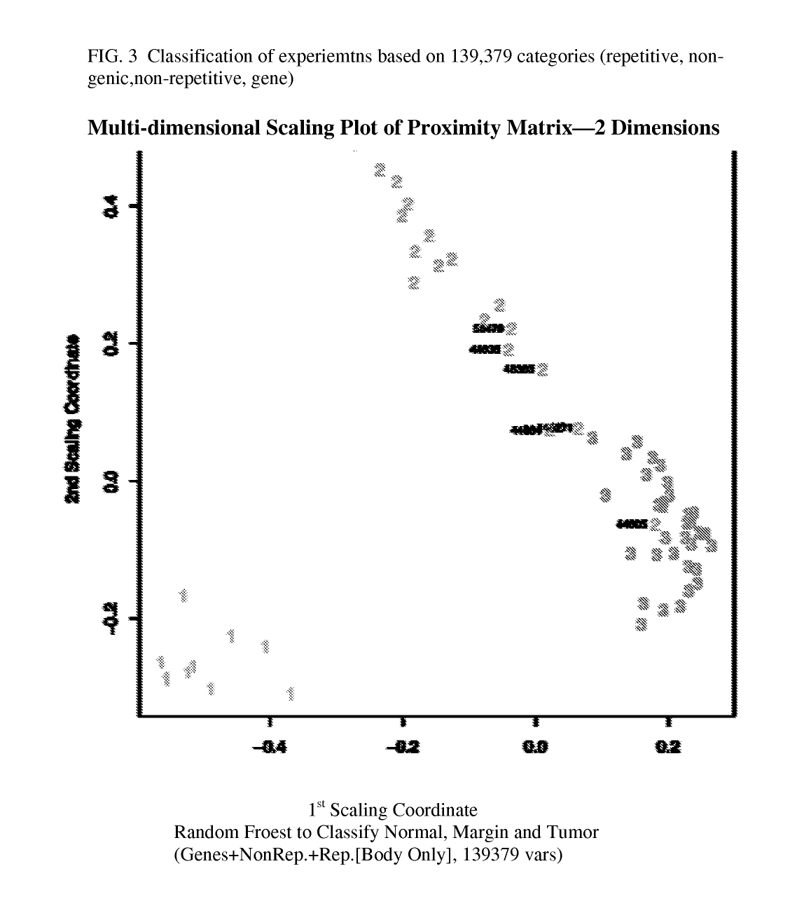

A list of repetitive DNA subfamilies that comprises approximately 900 members was generated. A list of DNA methylation values calculated as the average methylation of each category or sub-category of repetitive element was generated, including reprotransposon-derived elements, and DNA-transposon-derive elements. The values are obtained for individual experiments, and each average is generated my multiple probes of the same category. Two independent algorithms were used to rank the variables based on their abilities to classify experiments. Wilcoxon was used to classify tumor and non-tumor adjacent. Random Forest was used to classify Normal, Non-Tumor Adjacent and Tumor experiments. Both algorithms relied on the same definition of variables. The variables included single probes, or collections of probes sharing a common feature i.e. proximity to the repetitive element. Both algorithms ranked the variables based on repetitive-elements and non-genic, non-repetitive probes very high. Moreover, the repetitive element categories appear to be better classifiers than the gene probes as evidenced by the enrichment of repetitive element categories in the top ranked categories. Specifically, in top 30 categories there were 7 gene probes (out of ˜44,000), and 14 repetitive element categories (out of a total of 896) (FIG. 2).

The Wilcoxon test results, where the biomarker is ranked based on Wilcoxon test p-value for the top 200 variables out of 138,783 (repetitive elements, genes, non-genic, non-repetitive) are shown in Table 3. The Wilcoxon test results for the top 200 out of 90,007 non-repetitive non-gene probes are shown in Table 4. The Wilcoxon test results for all repetitive categories and literature-based categories (898) are shown in Table 5.

TABLE 3
Top 200 variables out of 138,783 (repetitive elements, genes, non-genic,
non-repetitive) ordered by a Wilcoxon p-value. The list is ordered by a Wilcoxon p-value
(pval) indicating how informative a category is to distinguish Tumor and Non-Tumor-
Adjacent experiments. Rank column shows a relative position of a category in the list, K is
an internal DB id of a category, count shows how many probes are included in the category.
A short description of a category indicates whether a probe is near a gene, or in a non-
repetitive part of the genome. For repetitive elements the description includes the information
about repeat name, class and family as well as the number of repeats in the genome and mean
length. P-value was calculated using Wilcoxon non-parametric, non-paired test.

| rank | K | count | description | pval |
|---|---|---|---|---|
| 1 | K.nonrep.122208 | 1 | K.nonrep.122208 | 4.55E−12 |
| 2 | K.nonrep.102186 | 1 | K.nonrep.102186 | 1.06E−09 |
| 3 | K.nonrep.122209 | 1 | K.nonrep.122209 | 1.31E−09 |
| 4 | K.repcat.e&g.2008.body.300.M [symbol] | 91 | MLT2B2.ERVL.LTR.2019.[335] | 1.99E−09 |
| 5 | K.gene.122207 | 1 | K.gene.122207 | 3.65E−09 |
| 6 | K.repcat.e&g.2008.body.300.E [symbol] | 6129 | ERVL.ERVL.LTR.826.[422] | 5.37E−09 |
| 7 | K.nonrep.122204 | 1 | K.nonrep.122204 | 6.49E−09 |
| 8 | K.gene.270555 | 1 | K.gene.270555 | 1.13E−08 |
| 9 | K.repcat.e&g.2008.body.300.M [symbol] | 15574 | MaLR | 1.61E−08 |
| 10 | K.nonrep.258987 | 1 | K.nonrep.258987 | 1.61E−08 |
| 11 | K.repcat.e&g.2008.body.300.L [symbol] | 38991 | LTR | 1.92E−08 |
| 12 | K.repcat.e&g.2008.body.300.M [symbol] | 239 | MLT1E2.MaLR.LTR.3960.[377] | 1.92E−08 |
| 13 | K.gene.152861 | 1 | K.gene.152861 | 1.92E−08 |
| 14 | K.gene.50699 | 1 | K.gene.50699 | 1.92E−08 |
| 15 | K.nonrep.147761 | 1 | K.nonrep.147761 | 1.92E−08 |
| 16 | K.gene.152859 | 1 | K.gene.152859 | 2.28E−08 |
| 17 | K.nonrep.147762 | 1 | K.nonrep.147762 | 2.28E−08 |
| 18 | K.nonrep.149154 | 1 | K.nonrep.149154 | 2.28E−08 |
| 19 | K.repcat.e.2008.body.300.MLT [symbol] | 420 | MLT1A.MaLR.LTR.8650.[280] | 2.71E−08 |
| 20 | K.gene.269730 | 1 | K.gene.269730 | 2.71E−08 |
| 21 | K.nonrep.19670 | 1 | K.nonrep.19670 | 2.71E−08 |
| 22 | K.repcat.e.2008.body.300.MLT [symbol] | 131 | MLT2B3.ERVL.LTR.2966.[406] | 3.21E−08 |
| 23 | K.nonrep.296142 | 1 | K.nonrep.296142 | 3.79E−08 |
| 24 | K.gene.298679 | 1 | K.gene.298679 | 5.25E−08 |
| 25 | K.gene.334064 | 1 | K.gene.334064 | 5.25E−08 |
| 26 | K.nonrep.122203 | 1 | K.nonrep.122203 | 6.17E−08 |
| 27 | K.repcat.e&g.2008.body.300.H [symbol] | 28 | HUERS-P3b.ERV1.LTR.378.[917] | 7.24E−08 |
| 28 | K.gene.223221 | 1 | K.gene.223221 | 7.24E−08 |
| 29 | K.nonrep.113501 | 1 | K.nonrep.113501 | 8.47E−08 |
| 30 | K.gene.122206 | 1 | K.gene.122206 | 9.90E−08 |
| 31 | K.gene.167356 | 1 | K.gene.167356 | 9.90E−08 |
| 32 | K.gene.228738 | 1 | K.gene.228738 | 9.90E−08 |
| 33 | K.nonrep.311545 | 1 | K.nonrep.311545 | 9.90E−08 |
| 34 | K.repcat.e.2008.body.300.MER [symbol] | 101 | MER50B.ERV1.LTR.681.[468] | 1.15E−07 |
| 35 | K.repcat.e.2008.body.300.MLT [symbol] | 236 | MLT1G1.MaLR.LTR.4437.[320] | 1.15E−07 |
| 36 | K.nonrep.153897 | 1 | K.nonrep.153897 | 1.15E−07 |
| 37 | K.repcat.e&g.2008.body.300.M [symbol] | 348 | MADE1.Mariner.DNA.7773.[61] | 1.34E−07 |
| 38 | K.repcat.e.2008.body.300.MER [symbol] | 216 | MER39.ERV1.LTR.3125.[336] | 1.34E−07 |
| 39 | K.standard.L1P.special | 740 | special | 1.34E−07 |
| 40 | K.gene.209707 | 1 | K.gene.209707 | 1.34E−07 |
| 41 | K.gene.34722 | 1 | K.gene.34722 | 1.34E−07 |
| 42 | K.gene.84633 | 1 | K gene.84633 | 1.34E−07 |
| 43 | K.gene.87798 | 1 | K.gene.87798 | 1.34E−07 |
| 44 | K.nonrep.227488 | 1 | K.nonrep.227488 | 1.34E−07 |
| 45 | K.nonrep.70135 | 1 | K.nonrep.70135 | 1.34E−07 |
| 46 | K.repcat.e&g.2008.body.300.M [symbol] | 169 | MLT2B1.ERVL.LTR.4186.[325] | 1.56E−07 |
| 47 | K.repcat.e.2008.body.300.MLT [symbol] | 1018 | MLT1A0.MaLR.LTR.20156.[279] | 1.56E−07 |
| 48 | K.gene.38080 | 1 | K.gene.38080 | 1.56E−07 |
| 49 | K.repcat.e.2008.body.300.MST [symbol] | 199 | MSTC.MaLR.LTR.3115.[284] | 1.81E−07 |
| 50 | K.gene.122205 | 1 | K.gene.122205 | 1.81E−07 |
| 51 | K.gene.138996 | 1 | K.gene.138996 | 1.81E−07 |
| 52 | K.nonrep.154468 | 1 | K.nonrep.154468 | 1.81E−07 |
| 53 | K.gene.50700 | 1 | K.gene.50700 | 2.10E−07 |
| 54 | K.nonrep.169335 | 1 | K.nonrep.169335 | 2.10E−07 |
| 55 | K.nonrep.38935 | 1 | K.nonrep.38935 | 2.43E−07 |
| 56 | K.repcat.e&g.2008.body.300.A [symbol] | 997 | AcHobo | 2.81E−07 |
| 57 | K.repcat.e.2008.body.300.MER [symbol] | 46 | MER66C.ERV1.LTR.661.[279] | 2.81E−07 |
| 58 | K.gene.170821 | 1 | K.gene.170821 | 2.81E−07 |
| 59 | K.gene.248792 | 1 | K.gene.248792 | 2.81E−07 |
| 60 | K.gene.57291 | 1 | K.gene.57291 | 2.81E−07 |
| 61 | K.gene.6904 | 1 | K.gene.6904 | 2.81E−07 |
| 62 | K.gene.77200 | 1 | K.gene.77200 | 2.81E−07 |
| 63 | K.gene.89746 | 1 | K.gene.89746 | 2.81E−07 |
| 64 | K.gene.99966 | 1 | K.gene.99966 | 2.81E−07 |
| 65 | K.nonrep.169334 | 1 | K.nonrep.169334 | 2.81E−07 |
| 66 | K.nonrep.230392 | 1 | K.nonrep.230392 | 2.81E−07 |
| 67 | K.nonrep.70785 | 1 | K.nonrep.70785 | 3.24E−07 |
| 68 | K.repcat.e&g.2008.body.300.L [symbol] | 53 | LTR54B.ERV1.LTR.832.[289] | 3.74E−07 |
| 69 | K.gene.232742 | 1 | K.gene.232742 | 3.74E−07 |
| 70 | K.gene.81226 | 1 | K.gene.81226 | 3.74E−07 |
| 71 | K.nonrep.101592 | 1 | K.nonrep.101592 | 3.74E−07 |
| 72 | K.nonrep.266307 | 1 | K.nonrep.266307 | 3.74E−07 |
| 73 | K.repcat.e&g.2008.body.300.M [symbol] | 49 | MER67D.ERV1.LTR.1130.[294] | 4.30E−07 |
| 74 | K.repcat.e&g.2008.body.300.M [symbol] | 174 | MLT2D.ERVL.LTR.4035.[266] | 4.30E−07 |
| 75 | K.repcat.e.2008.body.300.MLT [symbol] | 136 | MLT1E.MaLR.LTR.3378.[359] | 4.30E−07 |
| 76 | K.repcat.e.2008.body.300.MLT [symbol] | 506 | MLT1I.MaLR.LTR.11191.[218] | 4.30E−07 |
| 77 | K.gene.147633 | 1 | K.gene.147633 | 4.30E−07 |
| 78 | K.gene.153333 | 1 | K.gene.153333 | 4.30E−07 |
| 79 | K.gene.223222 | 1 | K.gene.223222 | 4.30E−07 |
| 80 | K.gene.226262 | 1 | K.gene.226262 | 4.30E−07 |
| 81 | K.gene.301073 | 1 | K.gene.301073 | 4.30E−07 |
| 82 | K.gene.303432 | 1 | K.gene.303432 | 4.30E−07 |
| 83 | K.nonrep.154467 | 1 | K.nonrep.154467 | 4.30E−07 |
| 84 | K.nonrep.274801 | 1 | K.nonrep.274801 | 4.30E−07 |
| 85 | K.nonrep.311662 | 1 | K.nonrep.311662 | 4.30E−07 |
| 86 | K.nonrep.53469 | 1 | K.nonrep.53469 | 4.30E−07 |
| 87 | K.repcat.e&g.2008.body.300.T [symbol] | 306 | Tigger3 (Golem).MER2 type.DNA [symbol] | 4.94E−07 |
| 88 | K.repcat.e.2008.body.300.THE [symbol] | 693 | THE1B.MaLR.LTR.22106.[329] | 4.94E−07 |
| 89 | K.gene.167548 | 1 | K.gene.167548 | 4.94E−07 |
| 90 | K.gene.285109 | 1 | K.gene.285109 | 4.94E−07 |
| 91 | K.nonrep.69552 | 1 | K.nonrep.69552 | 4.94E−07 |
| 92 | K.gene.27403 | 1 | K.gene.27403 | 5.67E−07 |
| 93 | K.gene.301951 | 1 | K.gene.301951 | 5.67E−07 |
| 94 | K.nonrep.266864 | 1 | K.nonrep.266864 | 5.67E−07 |
| 95 | K.repcat.e&g.2008.body.300.M [symbol] | 127 | MER63A.AcHobo.DNA.2791.[184] | 6.50E−07 |
| 96 | K.repcat.e.2008.body.300.MLT [symbol] | 219 | MLT1F2.MaLR.LTR.4152.[353] | 6.50E−07 |
| 97 | K.gene.102175 | 1 | K.gene.102175 | 6.50E−07 |
| 98 | K.gene.151208 | 1 | K.gene.151208 | 6.50E−07 |
| 99 | K.gene.174556 | 1 | K.gene.174556 | 6.50E−07 |
| 100 | K.gene.303208 | 1 | K.gene.303208 | 6.50E−07 |
| 101 | K.nonrep.266780 | 1 | K.nonrep.266780 | 6.50E−07 |
| 102 | K.repcat.e&g.2008 body.300.L [symbol] | 329 | L1PB4.L1.LINE.6330.[529] | 7.44E−07 |
| 103 | K.repcat.e&g.2008.body.300.L [symbol] | 10 | LSAU.Satellite.Satellite.41. [symbol] | 7.44E−07 |
| 104 | K.repcat.e.2008.body.300.MLT [symbol] | 199 | MLT1F1.MaLR.LTR.3489.[292] | 7.44E−07 |
| 105 | K.gene.296128 | 1 | K.gene.296128 | 7.44E−07 |
| 106 | K.gene.31409 | 1 | K.gene.31409 | 7.44E−07 |
| 107 | K.gene.54663 | 1 | K.gene.54663 | 7.44E−07 |
| 108 | K.gene.83279 | 1 | K.gene.83279 | 7.44E−07 |
| 109 | K.repcat.e.2008.body.300.ERV [symbol] | 132 | ERVL-B4.ERVL.LTR.2984.[564] | 8.51E−07 |
| 110 | K.repcat.e.2008.body.300.MER [symbol] | 80 | MER6.MER2 type.DNA.926.[418] | 8.51E−07 |
| 111 | K.repcat.e.2008.body.300.MER [symbol] | 82 | MER61A.ERV1.LTR.791.[303] | 8.51E−07 |
| 112 | K.gene.115654 | 1 | K.gene.115654 | 8.51E−07 |
| 113 | K.gene.133995 | 1 | K.gene.133995 | 8.51E−07 |
| 114 | K.gene.191733 | 1 | K.gene.191733 | 8.51E−07 |
| 115 | K.gene.192817 | 1 | K.gene.192817 | 8.51E−07 |
| 116 | K.gene.203055 | 1 | K.gene.203055 | 8.51E−07 |
| 117 | K.gene.219208 | 1 | K.gene.219208 | 8.51E−07 |
| 118 | K.gene.227873 | 1 | K.gene.227873 | 8.51E−07 |
| 119 | K.gene.315989 | 1 | K.gene.315989 | 8.51E−07 |
| 120 | K.gene.54860 | 1 | K.gene.54860 | 8.51E−07 |
| 121 | K.gene.61805 | 1 | K.gene.61805 | 8.51E−07 |
| 122 | K.gene.63060 | 1 | K.gene.63060 | 8.51E−07 |
| 123 | K.nonrep.15878 | 1 | K.nonrep.15878 | 8.51E−07 |
| 124 | K.nonrep.7463 | 1 | K.nonrep.7463 | 8.51E−07 |
| 125 | K.nonrep.90539 | 1 | K.nonrep.90539 | 8.51E−07 |
| 126 | K.repcat.e.2008.body.300.MLT [symbol] | 307 | MLT1A1.MaLR.LTR.6611.[294] | 9.71E−07 |
| 127 | K.gene.152252 | 1 | K.gene.152252 | 9.71E−07 |
| 128 | K.gene.182820 | 1 | K.gene.182820 | 9.71E−07 |
| 129 | K.gene.232740 | 1 | K.gene.232740 | 9.71E−07 |
| 130 | K.nonrep.211225 | 1 | K.nonrep.211225 | 9.71E−07 |
| 131 | K.repcat.e&g.2008.body.300.L [symbol] | 881 | L1MC3.L1.LINE.12792.[547] | 1.11E−06 |
| 132 | K.gene.174505 | 1 | K.gene.174505 | 1.11E−06 |
| 133 | K.gene.252637 | 1 | K.gene.252637 | 1.11E−06 |
| 134 | K.gene.268533 | 1 | K.gene.268533 | 1.11E−06 |
| 135 | K.gene.295375 | 1 | K.gene.295375 | 1.11E−06 |
| 136 | K.gene.61963 | 1 | K.gene.61963 | 1.11E−06 |
| 137 | K.nonrep.128685 | 1 | K.nonrep.128685 | 1.11E−06 |
| 138 | K.nonrep.266306 | 1 | K.nonrep.266306 | 1.11E−06 |
| 139 | K.nonrep.295372 | 1 | K.nonrep.295372 | 1.11E−06 |
| 140 | K.gene.102177 | 1 | K.gene.102177 | 1.26E−06 |
| 141 | K.gene.111133 | 1 | K.gene.111133 | 1.26E−06 |
| 142 | K.gene.174541 | 1 | K.gene.174541 | 1.26E−06 |
| 143 | K.gene.328463 | 1 | K.gene.328463 | 1.26E−06 |
| 144 | K.gene.99987 | 1 | K.gene.99987 | 1.26E−06 |
| 145 | K.nonrep.110206 | 1 | K.nonrep.110206 | 1.26E−06 |
| 146 | K.nonrep.14883 | 1 | K.nonrep.14883 | 1.26E−06 |
| 147 | K.nonrep.163948 | 1 | K.nonrep.163948 | 1.26E−06 |
| 148 | K.nonrep.180551 | 1 | K.nonrep.180551 | 1.26E−06 |
| 149 | K.nonrep.211798 | 1 | K.nonrep.211798 | 1.26E−06 |
| 150 | K.repcat.e&g.2008.body.300.M [symbol] | 855 | Mariner | 1.43E−06 |
| 151 | K.repcat.e.2008.body.300. ERV [symbol] | 33 | ERV | 1.43E−06 |
| 152 | K.gene.130982 | 1 | K.gene.130982 | 1.43E−06 |
| 153 | K.gene.319750 | 1 | K.gene.319750 | 1.43E−06 |
| 154 | K.gene.38011 | 1 | K.gene.38011 | 1.43E−06 |
| 155 | K.gene.41734 | 1 | K.gene.41734 | 1.43E−06 |
| 156 | K.gene.74207 | 1 | K.gene.74207 | 1.43E−06 |
| 157 | K.gene.85137 | 1 | K.gene.85137 | 1.43E−06 |
| 158 | K.nonrep.151268 | 1 | K.nonrep.151268 | 1.43E−06 |
| 159 | K.nonrep.168488 | 1 | K.nonrep.168488 | 1.43E−06 |
| 160 | K.nonrep.229863 | 1 | K.nonrep.229863 | 1.43E−06 |
| 161 | K.nonrep.278715 | 1 | K.nonrep.278715 | 1.43E−06 |
| 162 | K.nonrep.69598 | 1 | K.nonrep.69598 | 1.43E−06 |
| 163 | K.nonrep.96781 | 1 | K.nonrep.96781 | 1.43E−06 |
| 164 | K.repcat.e&g.2008.body.300.L [symbol] | 15 | LTR22A.ERVK.LTR.187.[402] | 1.63E−06 |
| 165 | K.gene.132656 | 1 | K.gene.132656 | 1.63E−06 |
| 166 | K.gene.237832 | 1 | K.gene.237832 | 1.63E−06 |
| 167 | K.gene.261415 | 1 | K.gene.261415 | 1.63E−06 |
| 168 | K.gene.265132 | 1 | K.gene.265132 | 1.63E−06 |
| 169 | K.gene.271240 | 1 | K.gene.271240 | 1.63E−06 |
| 170 | K.gene.296981 | 1 | K.gene.296981 | 1.63E−06 |
| 171 | K.gene.327487 | 1 | K.gene.327487 | 1.63E−06 |
| 172 | K.nonrep.260355 | 1 | K.nonrep.260355 | 1.63E−06 |
| 173 | K.nonrep.308324 | 1 | K.nonrep.308324 | 1.63E−06 |
| 174 | K.nonrep.83323 | 1 | K.nonrep.83323 | 1.63E−06 |
| 175 | K.nonrep.83324 | 1 | K.nonrep.83324 | 1.63E−06 |
| 176 | K.repcat.e&g.2008.body.300.L [symbol] | 108 | LTR40a.ERVL.LTR.2060.[328] | 1.85E−06 |
| 177 | K.gene.165048 | 1 | K.gene.165048 | 1.85E−06 |
| 178 | K.gene.183908 | 1 | K.gene.183908 | 1.85E−06 |
| 179 | K.gene.76851 | 1 | K.gene.76851 | 1.85E−06 |
| 180 | K.nonrep.100314 | 1 | K.nonrep.100314 | 1.85E−06 |
| 181 | K.nonrep.14898 | 1 | K.nonrep.14898 | 1.85E−06 |
| 182 | K.nonrep.162733 | 1 | K.nonrep.162733 | 1.85E−06 |
| 183 | K.nonrep.229975 | 1 | K.nonrep.229975 | 1.85E−06 |
| 184 | K.nonrep.232745 | 1 | K.nonrep.232745 | 1.85E−06 |
| 185 | K.nonrep.264903 | 1 | K.nonrep.264903 | 1.85E−06 |
| 186 | K.nonrep.303433 | 1 | K.nonrep.303433 | 1.85E−06 |
| 187 | K.nonrep.69336 | 1 | K.nonrep.69336 | 1.85E−06 |
| 188 | K.nonrep.86313 | 1 | K.nonrep.86313 | 1.85E−06 |
| 189 | K.repcat.e&g.2008.body.300.L [symbol] | 383 | L1MA7.L1.LINE.7550.[459] | 2.10E−06 |
| 190 | K.repcat.e.2008.body.300.MER [symbol] | 217 | MER41A-ERV1-LTR-2479.[384] | 2.10E−06 |
| 191 | K.gene.123586 | 1 | K.gene.123586 | 2.10E−06 |
| 192 | K.gene.146588 | 1 | K.gene.146588 | 2.10E−06 |
| 193 | K.gene.31528 | 1 | K.gene.31528 | 2.10E−06 |
| 194 | K.gene.330104 | 1 | K.gene.330104 | 2.10E−06 |
| 195 | K.gene.43145 | 1 | K.gene.43145 | 2.10E−06 |
| 196 | K.gene.51985 | 1 | K.gene.51985 | 2.10E−06 |
| 197 | K.nonrep.149152 | 1 | K.nonrep.149152 | 2.10E−06 |
| 198 | K.nonrep.221153 | 1 | K.nonrep.221153 | 2.10E−06 |
| 199 | K.nonrep.265143 | 1 | K.nonrep.265143 | 2.10E−06 |
| 200 | K.nonrep.309324 | 1 | K.nonrep.309324 | 2.10E−06 |

[symbol]

indicates data missing or illegible when filed

TABLE 4
Top 200 out of 90,007 non-repetitive non-gene probes ranked
based on non-paired Wilcoxon test. Based on non-paired
Wilcoxon test. these best differentiate Tumor from Non-Tumor
Adjacent experiments. K and description indicate internal probe
id of the category, count indicates that the category is a single probe.

| rank | K | count | description | pval |
|---|---|---|---|---|
| 1 | K.nonrep.122208 | 1 | K.nonrep.122208 | 4.55E−12 |
| 2 | K.nonrep.102186 | 1 | K.nonrep.102186 | 1.06E−09 |
| 3 | K.nonrep.122209 | 1 | K.nonrep.122209 | 1.31E−09 |
| 4 | K.nonrep.122204 | 1 | K.nonrep.122204 | 6.49E−09 |
| 5 | K.nonrep.258987 | 1 | K.nonrep.258987 | 1.61E−08 |
| 6 | K.nonrep.147761 | 1 | K.nonrep.147761 | 1.92E−08 |
| 7 | K.nonrep.147762 | 1 | K.nonrep.147762 | 2.28E−08 |
| 8 | K.nonrep.149154 | 1 | K.nonrep.149154 | 2.28E−08 |
| 9 | K.nonrep.19670 | 1 | K.nonrep.19670 | 2.71E−08 |
| 10 | K.nonrep.296142 | 1 | K.nonrep.296142 | 3.79E−08 |
| 11 | K.nonrep.122203 | 1 | K.nonrep.122203 | 6.17E−08 |
| 12 | K.nonrep.113501 | 1 | K.nonrep.113501 | 8.47E−08 |
| 13 | K.nonrep.311545 | 1 | K.nonrep.311545 | 9.90E−08 |
| 14 | K.nonrep.153897 | 1 | K.nonrep.153897 | 1.15E−07 |
| 15 | K.nonrep.227488 | 1 | K.nonrep.227488 | 1.34E−07 |
| 16 | K.nonrep.70135 | 1 | K.nonrep.70135 | 1.34E−07 |
| 17 | K.nonrep.154468 | 1 | K.nonrep.154468 | 1.81E−07 |
| 18 | K.nonrep.169335 | 1 | K.nonrep.169335 | 2.10E−07 |
| 19 | K.nonrep.38935 | 1 | K.nonrep.38935 | 2.43E−07 |
| 20 | K.nonrep.169334 | 1 | K.nonrep.169334 | 2.81E−07 |
| 21 | K.nonrep.230392 | 1 | K.nonrep.230392 | 2.81E−07 |
| 22 | K.nonrep.70785 | 1 | K.nonrep.70785 | 3.24E−07 |
| 23 | K.nonrep.101592 | 1 | K.nonrep.101592 | 3.74E−07 |
| 24 | K.nonrep.266307 | 1 | K.nonrep.266307 | 3.74E−07 |
| 25 | K.nonrep.154467 | 1 | K.nonrep.154467 | 4.30E−07 |
| 26 | K.nonrep.274801 | 1 | K.nonrep.274801 | 4.30E−07 |
| 27 | K.nonrep.311662 | 1 | K.nonrep.311662 | 4.30E−07 |
| 28 | K.nonrep.53469 | 1 | K.nonrep.53469 | 4.30E−07 |
| 29 | K.nonrep.69552 | 1 | K.nonrep.69552 | 4.94E−07 |
| 30 | K.nonrep.266864 | 1 | K.nonrep.266864 | 5.67E−07 |
| 31 | K.nonrep.266780 | 1 | K.nonrep.266780 | 6.50E−07 |
| 32 | K.nonrep.15878 | 1 | K.nonrep.15878 | 8.51E−07 |
| 33 | K.nonrep.7463 | 1 | K.nonrep.7463 | 8.51E−07 |
| 34 | K.nonrep.90539 | 1 | K.nonrep.90539 | 8.51E−07 |
| 35 | K.nonrep.211225 | 1 | K.nonrep.211225 | 9.71E−07 |
| 36 | K.nonrep.128685 | 1 | K.nonrep.128685 | 1.11E−06 |
| 37 | K.nonrep.266306 | 1 | K.nonrep.266306 | 1.11E−06 |
| 38 | K.nonrep.295372 | 1 | K.nonrep.295372 | 1.11E−06 |
| 39 | K.nonrep.110206 | 1 | K.nonrep.110206 | 1.26E−06 |
| 40 | K.nonrep.14883 | 1 | K.nonrep.14883 | 1.26E−06 |
| 41 | K.nonrep.163948 | 1 | K.nonrep.163948 | 1.26E−06 |
| 42 | K.nonrep.180551 | 1 | K.nonrep.180551 | 1.26E−06 |
| 43 | K.nonrep.211798 | 1 | K.nonrep.211798 | 1.26E−06 |
| 44 | K.nonrep.151268 | 1 | K.nonrep.151268 | 1.43E−06 |
| 45 | K.nonrep.168488 | 1 | K.nonrep.168488 | 1.43E−06 |
| 46 | K.nonrep.229863 | 1 | K.nonrep.229863 | 1.43E−06 |
| 47 | K.nonrep.278715 | 1 | K.nonrep.278715 | 1.43E−06 |
| 48 | K.nonrep.69598 | 1 | K.nonrep.69598 | 1.43E−06 |
| 49 | K.nonrep.96781 | 1 | K.nonrep.96781 | 1.43E−06 |
| 50 | K.nonrep.260355 | 1 | K.nonrep.260355 | 1.63E−06 |
| 51 | K.nonrep.308324 | 1 | K.nonrep.308324 | 1.63E−06 |
| 52 | K.nonrep.83323 | 1 | K.nonrep.83323 | 1.63E−06 |
| 53 | K.nonrep.83324 | 1 | K.nonrep.83324 | 1.63E−06 |
| 54 | K.nonrep.100314 | 1 | K.nonrep.100314 | 1.85E−06 |
| 55 | K.nonrep.14898 | 1 | K.nonrep.14898 | 1.85E−06 |
| 56 | K.nonrep.162733 | 1 | K.nonrep.162733 | 1.85E−06 |
| 57 | K.nonrep.229975 | 1 | K.nonrep.229975 | 1.85E−06 |
| 58 | K.nonrep.232745 | 1 | K.nonrep.232745 | 1.85E−06 |
| 59 | K.nonrep.264903 | 1 | K.nonrep.264903 | 1.85E−06 |
| 60 | K.nonrep.303433 | 1 | K.nonrep.303433 | 1.85E−06 |
| 61 | K.nonrep.69336 | 1 | K.nonrep.69336 | 1.85E−06 |
| 62 | K.nonrep.86313 | 1 | K.nonrep.86313 | 1.85E−06 |
| 63 | K.nonrep.149152 | 1 | K.nonrep.149152 | 2.10E−06 |
| 64 | K.nonrep.221153 | 1 | K.nonrep.221153 | 2.10E−06 |
| 65 | K.nonrep.265143 | 1 | K.nonrep.265143 | 2.10E−06 |
| 66 | K.nonrep.309324 | 1 | K.nonrep.309324 | 2.10E−06 |
| 67 | K.nonrep.325327 | 1 | K.nonrep.325327 | 2.10E−06 |
| 68 | K.nonrep.83736 | 1 | K.nonrep.83736 | 2.10E−06 |
| 69 | K.nonrep.142003 | 1 | K.nonrep.142003 | 2.38E−06 |
| 70 | K.nonrep.149147 | 1 | K.nonrep.149147 | 2.38E−06 |
| 71 | K.nonrep.153222 | 1 | K.nonrep.153222 | 2.38E−06 |
| 72 | K.nonrep.236021 | 1 | K.nonrep.236021 | 2.38E−06 |
| 73 | K.nonrep.301887 | 1 | K.nonrep.301887 | 2.38E−06 |
| 74 | K.nonrep.313414 | 1 | K.nonrep.313414 | 2.38E−06 |
| 75 | K.nonrep.39461 | 1 | K.nonrep.39461 | 2.38E−06 |
| 76 | K.nonrep.168356 | 1 | K.nonrep.168356 | 2.69E−06 |
| 77 | K.nonrep.266868 | 1 | K.nonrep.266868 | 2.69E−06 |
| 78 | K.nonrep.298283 | 1 | K.nonrep.298283 | 2.69E−06 |
| 79 | K.nonrep.306097 | 1 | K.nonrep.306097 | 2.69E−06 |
| 80 | K.nonrep.33133 | 1 | K.nonrep.33133 | 2.69E−06 |
| 81 | K.nonrep.155005 | 1 | K.nonrep.155005 | 3.04E−06 |
| 82 | K.nonrep.191208 | 1 | K.nonrep.191208 | 3.04E−06 |
| 83 | K.nonrep.192962 | 1 | K.nonrep.192962 | 3.04E−06 |
| 84 | K.nonrep.280039 | 1 | K.nonrep.280039 | 3.04E−06 |
| 85 | K.nonrep.324931 | 1 | K.nonrep.324931 | 3.04E−06 |
| 86 | K.nonrep.36461 | 1 | K.nonrep.36461 | 3.04E−06 |
| 87 | K.nonrep.89681 | 1 | K.nonrep.89681 | 3.04E−06 |
| 88 | K.nonrep.91291 | 1 | K.nonrep.91291 | 3.04E−06 |
| 89 | K.nonrep.263088 | 1 | K.nonrep.263088 | 3.43E−06 |
| 90 | K.nonrep.285373 | 1 | K.nonrep.285373 | 3.43E−06 |
| 91 | K.nonrep.311342 | 1 | K.nonrep.311342 | 3.43E−06 |
| 92 | K.nonrep.1529 | 1 | K.nonrep.1529 | 3.86E−06 |
| 93 | K.nonrep.204771 | 1 | K.nonrep.204771 | 3.86E−06 |
| 94 | K.nonrep.230495 | 1 | K.nonrep.230495 | 3.86E−06 |
| 95 | K.nonrep.236959 | 1 | K.nonrep.236959 | 3.86E−06 |
| 96 | K.nonrep.261527 | 1 | K.nonrep.261527 | 3.86E−06 |
| 97 | K.nonrep.287123 | 1 | K.nonrep.287123 | 3.86E−06 |
| 98 | K.nonrep.328189 | 1 | K.nonrep.328189 | 3.86E−06 |
| 99 | K.nonrep.48142 | 1 | K.nonrep.48142 | 3.86E−06 |
| 100 | K.nonrep.109912 | 1 | K.nonrep.109912 | 4.35E−06 |
| 101 | K.nonrep.257391 | 1 | K.nonrep.257391 | 4.35E−06 |
| 102 | K.nonrep.281594 | 1 | K.nonrep.281594 | 4.35E−06 |
| 103 | K.nonrep.333017 | 1 | K.nonrep.333017 | 4.35E−06 |
| 104 | K.nonrep.34127 | 1 | K.nonrep.34127 | 4.35E−06 |
| 105 | K.nonrep.77018 | 1 | K.nonrep.77018 | 4.35E−06 |
| 106 | K.nonrep.149145 | 1 | K.nonrep.149145 | 4.90E−06 |
| 107 | K.nonrep.266856 | 1 | K.nonrep.266856 | 4.90E−06 |
| 108 | K.nonrep.312743 | 1 | K.nonrep.312743 | 4.90E−06 |
| 109 | K.nonrep.39460 | 1 | K.nonrep.39460 | 4.90E−06 |
| 110 | K.nonrep.55415 | 1 | K.nonrep.55415 | 4.90E−06 |
| 111 | K.nonrep.146703 | 1 | K.nonrep.146703 | 5.51E−06 |
| 112 | K.nonrep.166836 | 1 | K.nonrep.166836 | 5.51E−06 |
| 113 | K.nonrep.246999 | 1 | K.nonrep.246999 | 5.51E−06 |
| 114 | K.nonrep.32387 | 1 | K.nonrep.32387 | 5.51E−06 |
| 115 | K.nonrep.106801 | 1 | K.nonrep.106801 | 6.18E−06 |
| 116 | K.nonrep.112953 | 1 | K.nonrep.112953 | 6.18E−06 |
| 117 | K.nonrep.132542 | 1 | K.nonrep.132542 | 6.18E−06 |
| 118 | K.nonrep.138529 | 1 | K.nonrep.138529 | 6.18E−06 |
| 119 | K.nonrep.215454 | 1 | K.nonrep.215454 | 6.18E−06 |
| 120 | K.nonrep.217442 | 1 | K.nonrep.217442 | 6.18E−06 |
| 121 | K.nonrep.311326 | 1 | K.nonrep.311326 | 6.18E−06 |
| 122 | K.nonrep.315151 | 1 | K.nonrep.315151 | 6.18E−06 |
| 123 | K.nonrep.7464 | 1 | K.nonrep.7464 | 6.18E−06 |
| 124 | K.nonrep.8093 | 1 | K.nonrep.8093 | 6.18E−06 |
| 125 | K.nonrep.84002 | 1 | K.nonrep.84002 | 6.18E−06 |
| 126 | K.nonrep.91292 | 1 | K.nonrep.91292 | 6.18E−06 |
| 127 | K.nonrep.151013 | 1 | K.nonrep.151013 | 6.94E−06 |
| 128 | K.nonrep.180717 | 1 | K.nonrep.180717 | 6.94E−06 |
| 129 | K.nonrep.277169 | 1 | K.nonrep.277169 | 6.94E−06 |
| 130 | K.nonrep.37999 | 1 | K.nonrep.37999 | 6.94E−06 |
| 131 | K.nonrep.39495 | 1 | K.nonrep.39495 | 6.94E−06 |
| 132 | K.nonrep.61818 | 1 | K.nonrep.61818 | 6.94E−06 |
| 133 | K.nonrep.141416 | 1 | K.nonrep.141416 | 7.78E−06 |
| 134 | K.nonrep.141421 | 1 | K.nonrep.141421 | 7.78E−06 |
| 135 | K.nonrep.232140 | 1 | K.nonrep.232140 | 7.78E−06 |
| 136 | K.nonrep.247791 | 1 | K.nonrep.247791 | 7.78E−06 |
| 137 | K.nonrep.270015 | 1 | K.nonrep.270015 | 7.78E−06 |
| 138 | K.nonrep.296136 | 1 | K.nonrep.296136 | 7.78E−06 |
| 139 | K.nonrep.47698 | 1 | K.nonrep.47698 | 7.78E−06 |
| 140 | K.nonrep.98720 | 1 | K.nonrep.98720 | 7.78E−06 |
| 141 | K.nonrep.118789 | 1 | K.nonrep.118789 | 8.71E−06 |
| 142 | K.nonrep.145698 | 1 | K.nonrep.145698 | 8.71E−06 |
| 143 | K.nonrep.149151 | 1 | K.nonrep.149151 | 8.71E−06 |
| 144 | K.nonrep.153898 | 1 | K.nonrep.153898 | 8.71E−06 |
| 145 | K.nonrep.16216 | 1 | K.nonrep.16216 | 8.71E−06 |
| 146 | K.nonrep.250409 | 1 | K.nonrep.250409 | 8.71E−06 |
| 147 | K.nonrep.40381 | 1 | K.nonrep.40381 | 8.71E−06 |
| 148 | K.nonrep.134741 | 1 | K.nonrep.134741 | 9.75E−06 |
| 149 | K.nonrep.18185 | 1 | K.nonrep.18185 | 9.75E−06 |
| 150 | K.nonrep.211226 | 1 | K.nonrep.211226 | 9.75E−06 |
| 151 | K.nonrep.241555 | 1 | K.nonrep.241555 | 9.75E−06 |
| 152 | K.nonrep.246998 | 1 | K.nonrep.246998 | 9.75E−06 |
| 153 | K.nonrep.259070 | 1 | K.nonrep.259070 | 9.75E−06 |
| 154 | K.nonrep.262108 | 1 | K.nonrep.262108 | 9.75E−06 |
| 155 | K.nonrep.2880 | 1 | K.nonrep.2880 | 9.75E−06 |
| 156 | K.nonrep.314550 | 1 | K.nonrep.314550 | 9.75E−06 |
| 157 | K.nonrep.39202 | 1 | K.nonrep.39202 | 9.75E−06 |
| 158 | K.nonrep.102180 | 1 | K.nonrep.102180 | 1.09E−05 |
| 159 | K.nonrep.10262 | 1 | K.nonrep.10262 | 1.09E−05 |
| 160 | K.nonrep.103372 | 1 | K.nonrep.103372 | 1.09E−05 |
| 161 | K.nonrep.122219 | 1 | K.nonrep.122219 | 1.09E−05 |
| 162 | K.nonrep.192867 | 1 | K.nonrep.192867 | 1.09E−05 |
| 163 | K.nonrep.19642 | 1 | K.nonrep.19642 | 1.09E−05 |
| 164 | K.nonrep.26381 | 1 | K.nonrep.26381 | 1.09E−05 |
| 165 | K.nonrep.293297 | 1 | K.nonrep.293297 | 1.09E−05 |
| 166 | K.nonrep.312607 | 1 | K.nonrep.312607 | 1.09E−05 |
| 167 | K.nonrep.335925 | 1 | K.nonrep.335925 | 1.09E−05 |
| 168 | K.nonrep.38023 | 1 | K.nonrep.38023 | 1.09E−05 |
| 169 | K.nonrep.39395 | 1 | K.nonrep.39395 | 1.09E−05 |
| 170 | K.nonrep.60573 | 1 | K.nonrep.60573 | 1.09E−05 |
| 171 | K.nonrep.76028 | 1 | K.nonrep.76028 | 1.09E−05 |
| 172 | K.nonrep.77318 | 1 | K.nonrep.77318 | 1.09E−05 |
| 173 | K.nonrep.81488 | 1 | K.nonrep.81488 | 1.09E−05 |
| 174 | K.nonrep.82426 | 1 | K.nonrep.82426 | 1.09E−05 |
| 175 | K.nonrep.95015 | 1 | K.nonrep.95015 | 1.09E−05 |
| 176 | K.nonrep.98975 | 1 | K.nonrep.98975 | 1.09E−05 |
| 177 | K.nonrep.140999 | 1 | K.nonrep.140999 | 1.22E−05 |
| 178 | K.nonrep.175663 | 1 | K.nonrep.175663 | 1.22E−05 |
| 179 | K.nonrep.181213 | 1 | K.nonrep.181213 | 1.22E−05 |
| 180 | K.nonrep.192866 | 1 | K.nonrep.192866 | 1.22E−05 |
| 181 | K.nonrep.206705 | 1 | K.nonrep.206705 | 1.22E−05 |
| 182 | K.nonrep.291293 | 1 | K.nonrep.291293 | 1.22E−05 |
| 183 | K.nonrep.295083 | 1 | K.nonrep.295083 | 1.22E−05 |
| 184 | K.nonrep.311413 | 1 | K.nonrep.311413 | 1.22E−05 |
| 185 | K.nonrep.326403 | 1 | K.nonrep.326403 | 1.22E−05 |
| 186 | K.nonrep.333741 | 1 | K.nonrep.333741 | 1.22E−05 |
| 187 | K.nonrep.39359 | 1 | K.nonrep.39359 | 1.22E−05 |
| 188 | K.nonrep.45035 | 1 | K.nonrep.45035 | 1.22E−05 |
| 189 | K.nonrep.45700 | 1 | K.nonrep.45700 | 1.22E−05 |
| 190 | K.nonrep.51929 | 1 | K.nonrep.51929 | 1.22E−05 |
| 191 | K.nonrep.148110 | 1 | K.nonrep.148110 | 1.36E−05 |
| 192 | K.nonrep.195743 | 1 | K.nonrep.195743 | 1.36E−05 |
| 193 | K.nonrep.234061 | 1 | K.nonrep.234061 | 1.36E−05 |
| 194 | K.nonrep.292140 | 1 | K.nonrep.292140 | 1.36E−05 |
| 195 | K.nonrep.31569 | 1 | K.nonrep.31569 | 1.36E−05 |
| 196 | K.nonrep.318682 | 1 | K.nonrep.318682 | 1.36E−05 |
| 197 | K.nonrep.327365 | 1 | K.nonrep.327365 | 1.36E−05 |
| 198 | K.nonrep.328190 | 1 | K.nonrep.328190 | 1.36E−05 |
| 199 | K.nonrep.328628 | 1 | K.nonrep.328628 | 1.36E−05 |
| 200 | K.nonrep.54350 | 1 | K.nonrep.54350 | 1.36E−05 |

TABLE 5
All repetitive categories and literature-based categories (898) ranked
based on Wilcoxon test p-value. All 898 repetitive categories ranked based on Wilcoxon test
pvalue (pval) indicating how well a given category differentiates between Tumor and Non-
Tumor adjacent experiments. Description of a category provides information about repeat
name, class and family (based on RepBase and RepeatMasker) as well as a number of elements
in the genome and their [average length]. The number of probes used to create this category is
indicated in column count. The probes are found within the body of the repetitive element +/−300 bases.

| rank | K | count | description | pval |
|---|---|---|---|---|
| 1 | K.repcat.e&g.2008.body.300.M [symbol] | 91 | MLT2B2.ERVL.LTR.2019.[335] | 1.99E−09 |
| 2 | K.repcat.e&g.2008.body.300.E [symbol] | 6129 | ERVL.ERVL.LTR.826.[422] | 5.37E−09 |
| 3 | K.repcat.e&g.2008.body.300.M [symbol] | 15574 | MaLR | 1.61E−08 |
| 4 | K.repcat.e&g.2008.body.300.L [symbol] | 38991 | LTR | 1.92E−08 |
| 5 | K.repcat.e&g.2008.body.300.M [symbol] | 239 | MLT1E2.MaLR.LTR.3960.[377] | 1.92E−08 |
| 6 | K.repcat.e.2008.body.300.MLT [symbol] | 420 | MLT1A.MaLR.LTR.8650.[280] | 2.71E−08 |
| 7 | K.repcat.e.2008.body.300.MLT [symbol] | 131 | MLT2B3.ERVL.LTR.2966.[406] | 3.21E−08 |
| 8 | K.repcat.e&g.2008.body.300.H [symbol] | 28 | HUERS-P3b.ERV1.LTR.378.[917] | 7.24E−08 |
| 9 | K.repcat.e.2008.body.300.MER [symbol] | 101 | MER50B.ERV1.LTR.681.[468] | 1.15E−07 |
| 10 | K.repcat.e.2008.body.300.MLT [symbol] | 236 | MLT1G1.MaLR.LTR.4437.[320] | 1.15E−07 |
| 11 | K.repcat.e&g.2008.body.300.M [symbol] | 348 | MADE1.Mariner.DNA.7773.[61] | 1.34E−07 |
| 12 | K.repcat.e.2008.body.300.MER [symbol] | 216 | MER39.ERV1.LTR.3125.[336] | 1.34E−07 |
| 13 | K.repcat.e&g.2008.body.300.M [symbol] | 169 | MLT2B1.ERVL.LTR.4186.[325] | 1.56E−07 |
| 14 | K.repcat.e.2008.body.300.MLT [symbol] | 1018 | MLT1A0.MaLR.LTR.20156.[279] | 1.56E−07 |
| 15 | K.repcat.e.2008.body.300.MST [symbol] | 199 | MSTC.MaLR.LTR.3115.[284] | 1.81E−07 |
| 16 | K.repcat.e&g.2008.body.300.A [symbol] | 997 | AcHobo | 2.81E−07 |
| 17 | K.repcat.e.2008.body.300.MER [symbol] | 46 | MER66C.ERV1.LTR.661.[279] | 2.81E−07 |
| 18 | K.repcat.e&g.2008.body.300.L [symbol] | 53 | LTR54B.ERV1.LTR.832.[289] | 3.74E−07 |
| 19 | K.repcat.e&g.2008.body.300.M [symbol] | 49 | MER67D.ERV1.LTR.1130.[294] | 4.30E−07 |
| 20 | K.repcat.e&g.2008.body.300.M [symbol] | 174 | MLT2D.ERVL.LTR.4035.[266] | 4.30E−07 |
| 21 | K.repcat.e.2008.body.300.MLT [symbol] | 136 | MLT1E.MaLR.LTR.3378.[359] | 4.30E−07 |
| 22 | K.repcat.e.2008.body.300.MLT [symbol] | 506 | MLT1I.MaLR.LTR.11191.[218] | 4.30E−07 |
| 23 | K.repcat.e&g.2008.body.300.T [symbol] | 306 | Tigger3(Golem).MER2_type.DNA [symbol] | 4.94E−07 |
| 24 | K.repcat.e.2008.body.300.THE [symbol] | 693 | THE1B.MaLR.LTR.22106.[329] | 4.94E−07 |
| 25 | K.repcat.e&g.2008.body.300.M [symbol] | 127 | MER63A.AcHobo.DNA.2791.[184] | 6.50E−07 |
| 26 | K.repcat.e.2008.body.300.MLT [symbol] | 219 | MLT1F2.MaLR.LTR.4152.[353] | 6.50E−07 |
| 27 | K.repcat.e&g.2008.body.300.L [symbol] | 329 | L1PB4.L1.LINE.6330.[529] | 7.44E−07 |
| 28 | K.repcat.e&g.2008.body.300.L [symbol] | 10 | LSAU.Satellite.Satellite.41. [symbol] | 7.44E−07 |
| 29 | K.repcat.e.2008.body.300.MLT [symbol] | 199 | MLT1F1.MaLR.LTR.3489.[292] | 7.44E−07 |
| 30 | K.repcat.e.2008.body.300.ERV [symbol] | 132 | ERVL-B4.ERVL.LTR.2984.[564] | 8.51E−07 |
| 31 | K.repcat.e.2008.body.300.MER [symbol] | 80 | MER6.MER2_type.DNA.926.[418] | 8.51E−07 |
| 32 | K.repcat.e.2008.body.300.MER [symbol] | 82 | MER61A.ERV1.LTR.791.[303] | 8.51E−07 |
| 33 | K.repcat.e.2008.body.300.MLT [symbol] | 307 | MLT1A1.MaLR.LTR.6611.[294] | 9.71E−07 |
| 34 | K.repcat.e&g.2008.body.300.L [symbol] | 881 | L1MC3.L1.LINE.12792.[547] | 1.11E−06 |
| 35 | K.repcat.e&g.2008.body.300.M [symbol] | 855 | Mariner | 1.43E−06 |
| 36 | K.repcat.e.2008.body.300.ERV [symbol] | 33 | ERV | 1.43E−06 |
| 37 | K.repcat.e&g.2008.body.300.L [symbol] | 15 | LTR22A.ERVK.LTR.187.[402] | 1.63E−06 |
| 38 | K.repcat.e&g.2008.body.300.L [symbol] | 108 | LTR40a.ERVL.LTR.2060.[328] | 1.85E−06 |
| 39 | K.repcat.e&g.2008.body.300.L [symbol] | 383 | L1MA7.L1.LINE.7550.[459] | 2.10E−06 |
| 40 | K.repcat.e.2008.body.300.MER [symbol] | 217 | MER41A.ERV1.LTR.2479.[384] | 2.10E−06 |
| 41 | K.repcat.e&g.2008.body.300.C [symbol] | 57 | Charlie3.MER1_type.DNA.639.[ [symbol] | 2.38E−06 |
| 42 | K.repcat.e&g.2008.body.300.M [symbol] | 127 | MER57A- | 2.38E−06 |
| 43 | K.repcat.e.2008.body.300.HER [symbol] | 185 | HERV16.ERVL.LTR.4493.[505] | 2.38E−06 |
| 44 | K.repcat.e.2008.body.300.MER [symbol] | 108 | MER4D.ERV1.LTR.1275.[421] | 2.69E−06 |
| 45 | K.repcat.e&g.2008.body.300.M [symbol] | 43 | MER11A.ERVK.LTR.930.[920] | 3.04E−06 |
| 46 | K.repcat.e.2008.body.300.ERV [symbol] | 17 | ERVL-C1.ERVL.LTR.334.[700] | 3.04E−06 |
| 47 | K.repcat.e.2008.body.300.LTR [symbol] | 288 | LTR9.ERV1.LTR.1919.[459] | 3.04E−06 |
| 48 | K.repcat.e.2008.body.300.MLT [symbol] | 412 | MLT1H.MaLR.LTR.9741.[277] | 3.04E−06 |
| 49 | K.repcat.e&g.2008.body.300.E [symbol] | 451 | ERVL-E.ERVL.LTR.9525.[461] | 3.86E−06 |
| 50 | K.repcat.e&g.2008.body.300.M [symbol] | 157 | MLT2B4.ERVL.LTR.4120.[305] | 3.86E−06 |
| 51 | K.repcat.e.2008.body.300.MLT [symbol] | 112 | MLT1E1.MaLR.LTR.2001.[322] | 3.86E−06 |
| 52 | K.repcat.e.2008.body.300.THE [symbol] | 333 | THE1C.MaLR.LTR.9731.[329] | 3.86E−06 |
| 53 | K.repcat.e.2008.body.300.Zap [symbol] | 121 | Zaphod.Tip100.DNA.2063.[322] | 3.86E−06 |
| 54 | K.repcat.e&g.2008.body.300.L [symbol] | 475 | L1MC2.L1.LINE.5920.[505] | 4.35E−06 |
| 55 | K.repcat.e&g.2008.body.300.L [symbol] | 79198 | LINE | 4.35E−06 |
| 56 | K.repcat.e&g.2008.body.300.M [symbol] | 53 | MLT1F-int.MaLR.LTR.932.[387] | 4.35E−06 |
| 57 | K.repcat.e&g.2008.body.300.A [symbol] | 269 | Arthur1.Tip100.DNA.4836.[241 [symbol] | 4.90E−06 |
| 58 | K.repcat.e&g.2008.body.300.H [symbol] | 20 | HERVL-A2.ERVL.LTR.527.[1939] | 4.90E−06 |
| 59 | K.repcat.e&g.2008.body.300.H [symbol] | 49 | HERVL40.ERVL.LTR.1217.[378] | 5.51E−06 |
| 60 | K.repcat.e&g.2008.body.300.L [symbol] | 888 | L1MC1.L1.LINE.11868.[608] | 5.51E−06 |
| 61 | K.repcat.e&g.2008.body.300.L [symbol] | 14 | L1PBb.L1.LINE.271.[1050] | 5.51E−06 |
| 62 | K.repcat.e&g.2008.body.300.L [symbol] | 109 | LTR37A.ERV1.LTR.1957.[224] | 5.51E−06 |
| 63 | K.repcat.e.2008.body.300.MLT [symbol] | 796 | MLT1J.MaLR.LTR.15375.[246] | 5.51E−06 |
| 64 | K.repcat.e&g.2008.body.300.L [symbol] | 729 | L1MA9.L1.LINE.14394.[495] | 6.18E−06 |
| 65 | K.repcat.e.2008.body.300.LTR [symbol] | 90 | LTR49.ERV1.LTR.1406.[325] | 6.18E−06 |
| 66 | K.repcat.e&g.2008.body.300.L [symbol] | 560 | L1MA8.L1.LINE.9992.[497] | 6.94E−06 |
| 67 | K.repcat.e.2008.body.300.MST [symbol] | 349 | MSTD.MaLR.LTR.7421.[291] | 6.94E−06 |
| 68 | K.repcat.e.2008.body.300.MER [symbol] | 173 | MER57A.ERV1.LTR.2633.[290] | 7.78E−06 |
| 69 | K.repcat.e&g.2008.body.300.E [symbol] | 11 | ERVL-D.ERVL.LTR.249.[796] | 8.71E−06 |
| 70 | K.repcat.e&g.2008.body.300.L [symbol] | 507 | L1MD1.L1.LINE.6126.[612] | 8.71E−06 |
| 71 | K.repcat.e&g.2008.body.300.M [symbol] | 41 | MLT1A0- | 8.71E−06 |
| 72 | K.repcat.e.2008.body.300.MER [symbol] | 86 | MER41C.ERV1.LTR.799.[320] | 8.71E−06 |
| 73 | K.repcat.e.2008.body.300.MLT [symbol] | 213 | MLT1H1.MaLR.LTR.3868.[316] | 8.71E−06 |
| 74 | K.repcat.e&g.2008.body.300.L [symbol] | 300 | L1MB1.L1.LINE.5670.[521] | 9.75E−06 |
| 75 | K.repcat.e&g.2008.body.300.L [symbol] | 462 | L1MCa.L1.LINE.7557.[512] | 9.75E−06 |
| 76 | K.repcat.e.2008.body.300.L1M [symbol] | 220 | L1MA1.L1.LINE.3940.[1012] | 9.75E−06 |
| 77 | K.repcat.e.2008.body.300.LTR [symbol] | 109 | LTR1B.ERV1.LTR.1147.[622] | 9.75E−06 |
| 78 | K.repcat.e&g.2008.body.300.L [symbol] | 710 | L1MB4.L1.LINE.8956.[415] | 1.09E−05 |
| 79 | K.repcat.e.2008.body.300.THE [symbol] | 496 | THE1D.MaLR.LTR.12381.[324] | 1.09E−05 |
| 80 | K.repcat.e&g.2008.body.300.L [symbol] | 5 | LTR36-int.ERV1.LTR.71.[480] | 1.22E−05 |
| 81 | K.repcat.e&g.2008.body.300.H [symbol] | 100 | HSMAR1.Mariner.DNA.703.[661] | 1.36E−05 |
| 82 | K.repcat.e&g.2008.body.300.M [symbol] | 354 | MLT1J2.MaLR.LTR.7867.[215] | 1.36E−05 |
| 83 | K.repcat.e.2008.body.300.MST [symbol] | 872 | MSTA.MaLR.LTR.19102.[319] | 1.36E−05 |
| 84 | K.repcat.e&g.2008.body.300.L [symbol] | 944 | L1MD2.L1.LINE.12388.[485] | 1.51E−05 |
| 85 | K.repcat.e&g.2008.body.300.L [symbol] | 513 | L1PA13.L1.LINE.10722.[840] | 1.51E−05 |
| 86 | K.repcat.e&g.2008.body.300.L [symbol] | 26 | LTR55.ERV.LTR.402.[366] | 1.51E−05 |
| 87 | K.repcat.e.2008.body.300.L1 | 53617 | L1 | 1.51E−05 |
| 88 | K.repcat.e&g.2008.body.300.L [symbol] | 38 | LOR1-int.ERV1.LTR.561.[435] | 1.69E−05 |
| 89 | K.repcat.e&g.2008.body.300.L [symbol] | 41 | LTR22C.ERVK.LTR.378.[442] | 1.69E−05 |
| 90 | K.repcat.e&g.2008.body.300.M [symbol] | 136 | MLT2A2.ERVL.LTR.3754.[411] | 1.69E−05 |
| 91 | K.repcat.e&g.2008.body.300.D [symbol] | 24141 | DNA | 1.88E−05 |
| 92 | K.repcat.e&g.2008.body.300.T [symbol] | 902 | Tigger1.MER2_type.DNA.11928. [symbol] | 1.88E−05 |
| 93 | K.repcat.e&g.2008.body.300.L [symbol] | 307 | L1MCc.L1.LINE.6889.[452] | 2.09E−05 |
| 94 | K.repcat.e.2008.body.300.LTR [symbol] | 314 | LTR16A.ERVL.LTR.6873.[276] | 2.09E−05 |
| 95 | K.repcat.e&g.2008.body.300.M [symbol] | 11 | MER72B.ERV1.LTR.177.[394] | 2.32E−05 |
| 96 | K.repcat.e&g.2008.body.300.L [symbol] | 275 | L1MA10.L1.LINE.5419.[291] | 2.57E−05 |
| 97 | K.repcat.e&g.2008.body.300.M [symbol] | 114 | MLT2F.ERVL.LTR.1917.[323] | 2.57E−05 |
| 98 | K.repcat.e&g.2008.body.300.T [symbol] | 14 | THE1-int.MaLR.LTR.736.[300] | 2.57E−05 |
| 99 | K.repcat.e.2008.body.300.L1M [symbol] | 18 | L1M2a.L1.LINE.515.[883] | 2.57E−05 |
| 100 | K.repcat.e.2008.body.300.MER [symbol] | 35 | MER70A.ERVL.LTR.524.[386] | 2.57E−05 |
| 101 | K.repcat.e&g.2008.body.300.L [symbol] | 154 | L1MCb.L1.LINE.2863.[624] | 2.85E−05 |
| 102 | K.repcat.e&g.2008.body.300.L [symbol] | 1127 | L1ME3A.L1.LINE.16330.[362] | 2.85E−05 |
| 103 | K.repcat.e&g.2008.body.300.L [symbol] | 7 | LTR77.ERV1.LTR.149.[430] | 2.85E−05 |
| 104 | K.repcat.e&g.2008.body.300.t [symbol] | 10 | tRNA-Ile- | 2.85E−05 |
| 105 | K.repcat.e&g.2008.body.300.M [symbol] | 10 | MER66C- | 3.16E−05 |
| 106 | K.repcat.e&g.2008.body.300.t [symbol] | 1 | tRNA-Ile- | 3.16E−05 |
| 107 | K.repcat.e.2008.body.300.ERV [symbol] | 17485 | ERV1 | 3.16E−05 |
| 108 | K.repcat.e.2008.body.300.MER [symbol] | 287 | MER50.ERV1.LTR.2576.[431] | 3.16E−05 |
| 109 | K.repcat.e&g.2008.body.300.L [symbol] | 247 | L1MA6.L1.LINE.4693.[601] | 3.50E−05 |
| 110 | K.repcat.e&g.2008.body.300.L [symbol] | 2011 | L1ME1.L1.LINE.26938.[488] | 3.50E−05 |
| 111 | K.repcat.e&g.2008.body.300.L [symbol] | 288 | L1PA17.L1.LINE.4722.[700] | 3.50E−05 |
| 112 | K.repcat.e.2008.body.300.LTR [symbol] | 97 | LTR1D.ERV1.LTR.978.[707] | 3.50E−05 |
| 113 | K.repcat.e.2008.body.300.MER [symbol] | 18 | MER61C.ERV1.LTR.284.[360] | 3.50E−05 |
| 114 | K.repcat.e&g.2008.body.300.L [symbol] | 573 | L1MD3.L1.LINE.7482.[311] | 3.87E−05 |
| 115 | K.repcat.e&g.2008.body.300.L [symbol] | 723 | L1PA4.L1.LINE.11462.[1602] | 3.87E−05 |
| 116 | K.repcat.e&g.2008.body.300.M [symbol] | 148 | MLT2C1.ERVL.LTR.2455.[246] | 3.87E−05 |
| 117 | K.repcat.e.2008.body.300.MER [symbol] | 106 | MER4D1.ERV1.LTR.1393.[464] | 3.87E−05 |
| 118 | K.repcat.e.2008.body.300.MER [symbol] | 111 | MER9.ERVK.LTR.1128.[475] | 3.87E−05 |
| 119 | K.repcat.e&g.2008.body.300.L [symbol] | 454 | L1M4b.L1.LINE.7423.[511] | 4.28E−05 |
| 120 | K.repcat.e.2008.body.300.L1M [symbol] | 509 | L1MA4.L1.LINE.8983.[621] | 4.28E−05 |
| 121 | K.repcat.e.2008.body.300.LOR [symbol] | 65 | LOR1b.ERV1.LTR.1086.[334] | 4.28E−05 |
| 122 | K.repcat.e.2008.body.300.PRI [symbol] | 26 | PRIMA4_LTR.ERV1.LTR.344.[402 [symbol] | 4.28E−05 |
| 123 | K.repcat.e&g.2008.body.300.L [symbol] | 663 | L1PA16.L1.LINE.13098.[813] | 4.73E−05 |
| 124 | K.repcat.e&g.2008.body.300.M [symbol] | 106 | MER44B.MER2_type.DNA.1256.[3 [symbol] | 4.73E−05 |
| 125 | K.repcat.e&g.2008.body.300.M [symbol] | 16 | MLT2B5.ERVL.LTR.461.[240] | 4.73E−05 |
| 126 | K.repcat.e.2008.body.300.MER [symbol] | 137 | MER4B.ERV1.LTR.1709.[402] | 4.73E−05 |
| 127 | K.repcat.e&g.2008.body.300.H [symbol] | 14 | HERVL66.ERVL.LTR.104.[735] | 5.22E−05 |
| 128 | K.repcat.e&g.2008.body.300.H [symbol] | 32 | HUERS-P2.ERV1.LTR.242.[1082] | 5.22E−05 |
| 129 | K.repcat.e.2008.body.300.HUE [symbol] | 66 | HUERS-P3.ERV1.LTR.841.[806] | 5.22E−05 |
| 130 | K.repcat.e.2008.body.300.LTR [symbol] | 53 | LTR29.ERV1.LTR.667.[307] | 5.22E−05 |
| 131 | K.repcat.e.2008.body.300.MER [symbol] | 68 | MER57B.ERV1.LTR.1225.[343] | 5.22E−05 |
| 132 | K.repcat.e&g.2008.body.300.L [symbol] | 346 | L1MA4A.L1.LINE.5823.[836] | 5.76E−05 |
| 133 | K.repcat.e&g.2008.body.300.L [symbol] | 342 | L1PA10.L1.LINE.6962.[920] | 5.76E−05 |
| 134 | K.repcat.e&g.2008.body.300.M [symbol] | 34 | MSTD-int.MaLR.LTR.730.[812] | 5.76E−05 |
| 135 | K.repcat.e&g.2008.body.300.L [symbol] | 105 | L1P2.L1.LINE.2017.[1383] | 6.35E−05 |
| 136 | K.repcat.e&g.2008.body.300.L [symbol] | 5 | LTR27B- | 6.35E−05 |
| 137 | K.repcat.e&g.2008.body.300.M [symbol] | 31 | MER34B- | 6.35E−05 |
| 138 | K.repcat.e.2008.body.300.L1P [symbol] | 394 | L1PA15.L1.LINE.8233.[962] | 6.35E−05 |
| 139 | K.repcat.e.2008.body.300.MER [symbol] | 168 | MER34.ERV1.LTR.2364.[259] | 6.35E−05 |
| 140 | K.repcat.e&g.2008.body.300.M [symbol] | 30 | MER51B- | 6.99E−05 |
| 141 | K.repcat.e&g.2008.body.300.M [symbol] | 665 | MER58A.MER1_type.DNA.12543.[ [symbol] | 6.99E−05 |
| 142 | K.repcat.e&g.2008.body.300.M [symbol] | 9 | MER74C.ERVL.LTR.226.[323] | 6.99E−05 |
| 143 | K.repcat.e&g.2008.body.300.T [symbol] | 46 | THE1B- | 6.99E−05 |
| 144 | K.repcat.e&g.2008.body.300.T [symbol] | 196 | Tigger7.MER2_type.DNA.4405.[ [symbol] | 6.99E−05 |
| 145 | K.repcat.e.2008.body.300.MST [symbol] | 469 | MSTB.MaLR.LTR.8397.[321] | 6.99E−05 |
| 146 | K.repcat.e&g.2008.body.300.C [symbol] | 133 | Charlie10.MER1_type.DNA.2207 [symbol] | 7.69E−05 |
| 147 | K.repcat.e&g.2008.body.300.L [symbol] | 365 | L1HS.L1.LINE.1696.[2016] | 8.46E−05 |
| 148 | K.repcat.e&g.2008.body.300.L [symbol] | 190 | LTR16B.ERVL.LTR.3456.[292] | 8.46E−05 |
| 149 | K.repcat.e&g.2008.body.300.L [symbol] | 134 | LTR41.ERVL.LTR.2357.[234] | 8.46E−05 |
| 150 | K.repcat.e&g.2008.body.300.L [symbol] | 286 | LTR67.ERVL.LTR.6289.[175] | 8.46E−05 |
| 151 | K.repcat.e&g.2008.body.300.t [symbol] | 10 | tRNA-His- | 8.46E−05 |
| 152 | K.repcat.e.2008.body.300.MER [symbol] | 219 | MER63.AcHobo.DNA.3400.[139] | 8.46E−05 |
| 153 | K.repcat.e&g.2008.body.300.H [symbol] | 44 | HERVL-A1.ERVL.LTR.968.[2204] | 9.30E−05 |
| 154 | K.repcat.e&g.2008.body.300.L [symbol] | 182 | L1PB3.L1.LINE.3302.[827] | 9.30E−05 |
| 155 | K.repcat.e&g.2008.body.300.L [symbol] | 34 | LTR51.ERV1.LTR.399.[424] | 9.30E−05 |
| 156 | K.repcat.e&g.2008.body.300.L [symbol] | 14 | LTR75_1.ERV1.LTR.129.[320] | 9.30E−05 |
| 157 | K.repcat.e&g.2008.body.300.M [symbol] | 5338 | MER2_type | 9.30E−05 |
| 158 | K.repcat.e&g.2008.body.300.M [symbol] | 3 | MER66A- | 9.30E−05 |
| 159 | K.repcat.e&g.2008.body.300.M [symbol] | 118 | MSR1.Satellite.Satellite.318 [symbol] | 9.30E−05 |
| 160 | K.repcat.e.2008.body.300.L1P [symbol] | 117 | L1P.L1.LINE.1589.[520] | 9.30E−05 |
| 161 | K.repcat.e.2008.body.300.MER [symbol] | 243 | MER41B.ERV1.LTR.2864.[370] | 9.30E−05 |
| 162 | K.repcat.e.2008.body.300.MLT [symbol] | 1331 | MLT1D.MaLR.LTR.19943.[325] | 9.30E−05 |
| 163 | K.repcat.e&g.2008.body.300.L [symbol] | 64 | L1PA15- | 1.02E−04 |
| 164 | K.repcat.e&g.2008.body.300.L [symbol] | 507 | L1PA7.L1.LINE.12334.[1556] | 1.02E−04 |
| 165 | K.repcat.e.2008.body.300.HER [symbol] | 36 | HERVK11.ERVK.LTR.300.[1278] | 1.02E−04 |
| 166 | K.repcat.e.2008.body.300.LTR [symbol] | 274 | LTR12.ERV1.LTR.964.[633] | 1.02E−04 |
| 167 | K.repcat.e.2008.body.300.MER [symbol] | 34 | MER110.ERV1.LTR.736.[250] | 1.02E−04 |
| 168 | K.repcat.e&g.2008.body.300.L [symbol] | 183 | L1P1.L1.LINE.2860.[838] | 1.12E−04 |
| 169 | K.repcat.e&g.2008.body.300.L [symbol] | 193 | L1PA11.L1.LINE.3886.[997] | 1.12E−04 |
| 170 | K.repcat.e&g.2008.body.300.L [symbol] | 187 | L1PA14.L1.LINE.3561.[941] | 1.12E−04 |
| 171 | K.repcat.e&g.2008.body.300.M [symbol] | 65 | MER31- | 1.12E−04 |
| 172 | K.repcat.e.2008.body.300.LTR [symbol] | 331 | LTR8.ERV1.LTR.3520.[439] | 1.12E−04 |
| 173 | K.repcat.e.2008.body.300.MER [symbol] | 163 | MER4A1.ERV1.LTR.2150.[358] | 1.12E−04 |
| 174 | K.repcat.e&g.2008.body.300.L [symbol] | 182 | L1MA5A.L1.LINE.3165.[541] | 1.23E−04 |
| 175 | K.repcat.e&g.2008.body.300.L [symbol] | 167 | L1PB2.L1.LINE.2666.[831] | 1.23E−04 |
| 176 | K.repcat.e&g.2008.body.300.M [symbol] | 12 | MLT1A-int.MaLR.LTR.428.[612] | 1.23E−04 |
| 177 | K.repcat.e.2008.body.300.THE [symbol] | 156 | THE1A.MaLR.LTR.4161.[319] | 1.23E−04 |
| 178 | K.repcat.e.2008.body.300.HER [symbol] | 34 | HERVL.ERVL.LTR.1081.[547] | 1.35E−04 |
| 179 | K.repcat.e.2008.body.300.L1P [symbol] | 255 | L1P4.L1.LINE.5156.[529] | 1.35E−04 |
| 180 | K.repcat.e&g.2008.body.300.L [symbol] | 547 | L1PA5.L1.LINE.10904.[1449] | 1.47E−04 |
| 181 | K.repcat.e&g.2008.body.300.L [symbol] | 64 | LTR16D.ERVL.LTR.1747.[217] | 1.47E−04 |
| 182 | K.repcat.e&g.2008.body.300.T [symbol] | 444 | Tigger3b.MER2_type.DNA.4998. [symbol] | 1.47E−04 |
| 183 | K.repcat.e.2008.body.300.MER [symbol] | 101 | MER67C.ERV1.LTR.1468.[341] | 1.47E−04 |
| 184 | K.repcat.e&g.2008.body.300.L [symbol] | 174 | LTR33A.ERVL.LTR.3521.[226] | 1.61E−04 |
| 185 | K.repcat.e.2008.body.300.LTR [symbol] | 516 | LTR7.ERV1.LTR.2349.[407] | 1.61E−04 |
| 186 | K.repcat.e&g.2008.body.300.A [symbol] | 411 | AluYg6.Alu.SINE.677.[254] | 1.76E−04 |
| 187 | K.repcat.e&g.2008.body.300.L [symbol] | 59 | LTR47A.ERVL.LTR.873.[260] | 1.76E−04 |
| 188 | K.repcat.e&g.2008.body.300.M [symbol] | 151 | MLT1H- | 1.76E−04 |
| 189 | K.repcat.e.2008.body.300.MER [symbol] | 37 | MER67A.ERV1.LTR.484.[249] | 1.76E−04 |
| 190 | K.repcat.e&g.2008.body.300.L [symbol] | 73 | L1MC4_3endX.L1.LINE.1031.[19 [symbol] | 1.93E−04 |
| 191 | K.repcat.e&g.2008.body.300.M [symbol] | 28 | MER34-int.ERV1.LTR.394.[442] | 1.93E−04 |
| 192 | K.repcat.e&g.2008.body.300.M [symbol] | 17 | MLT1C-int.MaLR.LTR.421.[706] | 1.93E−04 |
| 193 | K.repcat.e.2008.body.300.Alu [symbol] | 15 | AluYd.Alu.SINE.65.[107] | 1.93E−04 |
| 194 | K.repcat.e.2008.body.300.L1M [symbol] | 452 | L1M3.L1.LINE.7700.[374] | 1.93E−04 |
| 195 | K.repcat.e.2008.body.300.MER [symbol] | 32 | MER4A1_.ERV1.LTR.380.[486] | 1.93E−04 |
| 196 | K.repcat.e.2008.body.300.MER [symbol] | 15 | MER61D.ERV1.LTR.263.[361] | 1.93E−04 |
| 197 | K.repcat.e&g.2008.body.300.M [symbol] | 14 | MER31B- | 2.11E−04 |
| 198 | K.repcat.e&g.2008.body.300.M [symbol] | 63 | MER4B-int.ERV1.LTR.983.[492] | 2.11E−04 |
| 199 | K.repcat.e&g.2008.body.300.M [symbol] | 199 | MER94.AcHobo.DNA.4680.[105] | 2.11E−04 |
| 200 | K.repcat.e&g.2008.body.300.t [symbol] | 2 | tRNA-Pro- | 2.11E−04 |
| 201 | K.repcat.e.2008.body.300.Alu [symbol] | 76382 | AluY.Alu.SINE.135761.[288] | 2.11E−04 |
| 202 | K.repcat.e&g.2008.body.300.L [symbol] | 75 | L1P5.L1.LINE.1531.[587] | 2.30E−04 |
| 203 | K.repcat.e&g.2008.body.300.L [symbol] | 1276 | L1PA3.L1.LINE.10281.[1836] | 2.30E−04 |
| 204 | K.repcat.e&g.2008.body.300.L [symbol] | 197 | LTR16A1.ERVL.LTR.3898.[252] | 2.30E−04 |
| 205 | K.repcat.e&g.2008.body.300.L [symbol] | 154 | LTR8A.ERV1.LTR.2663.[482] | 2.30E−04 |
| 206 | K.repcat.e&g.2008.body.300.M [symbol] | 41 | MER11B.ERVK.LTR.552.[932] | 2.30E−04 |
| 207 | K.repcat.e&g.2008.body.300.S [symbol] | 197952 | SINE | 2.30E−04 |
| 208 | K.repcat.e&g.2008.body.300.t [symbol] | 7 | tRNA-Asn- | 2.30E−04 |
| 209 | K.repcat.e&g.2008.body.300.A [symbol] | 13426 | AluSc.Alu.SINE.48789.[278] | 2.51E−04 |
| 210 | K.repcat.e&g.2008.body.300.L [symbol] | 1317 | L1MEc.L1.LINE.21328.[480] | 2.51E−04 |
| 211 | K.repcat.e&g.2008.body.300.M [symbol] | 37 | MER11D.ERVK.LTR.259.[745] | 2.51E−04 |
| 212 | K.repcat.e&g.2008.body.300.M [symbol] | 109 | MER45B.Tip100.DNA.1173.[403] | 2.51E−04 |
| 213 | K.repcat.e&g.2008.body.300.M [symbol] | 35 | MER57B- | 2.51E−04 |
| 214 | K.repcat.e&g.2008.body.300.t [symbol] | 241 | tRNA | 2.51E−04 |
| 215 | K.repcat.e.2008.body.300.MER [symbol] | 2231 | MER5A.MER1_type.DNA.35576.[1 [symbol] | 2.51E−04 |
| 216 | K.repcat.e.2008.body.300.Tig [symbol] | 112 | Tigger5.MER2_type.DNA.2895.[ [symbol] | 2.51E−04 |
| 217 | K.Sunami.PAPER | 749 | K. Sunami | 2.51E−04 |
| 218 | K.Yang.PAPER | 810 | K. Yang | 2.51E−04 |
| 219 | K.repcat.e&g.2008.body.300.L [symbol] | 19 | LTR22B.ERVK.LTR.219.[438] | 2.73E−04 |
| 220 | K.repcat.e&g.2008.body.300.L [symbol] | 44 | LTR66.ERVL.LTR.515.[216] | 2.73E−04 |
| 221 | K.repcat.e.2008.body.300.L1M [symbol] | 243 | L1MA5.L1.LINE.4086.[656] | 2.73E−04 |
| 222 | K.repcat.e.2008.body.300.L1P [symbol] | 205 | L1P3.L1.LINE.4518.[756] | 2.73E−04 |
| 223 | K.repcat.e.2008.body.300.MLT [symbol] | 97 | MLT1E3.MaLR.LTR.1701.[338] | 2.73E−04 |
| 224 | K.Chalitchagorn.PAPER | 729 | K. Chalitchagorn | 2.73E−04 |
| 225 | K.Woloszynska.PAPER | 486 | K. Woloszynska | 2.73E−04 |
| 226 | K.Rago.PAPER | 494 | K. Rago | 2.73E−04 |
| 227 | K.repcat.e&g.2008.body.300.L [symbol] | 13 | LTR19A-int.ERV1.LTR.99.[996] | 2.98E−04 |
| 228 | K.repcat.e&g.2008.body.300.M [symbol] | 917 | MER5A1.MER1_type.DNA.14831.[ [symbol] | 2.98E−04 |
| 229 | K.repcat.e.2008.body.300.L1M [symbol] | 463 | L1M2.L1.LINE.9352.[648] | 2.98E−04 |
| 230 | K.repcat.e.2008.body.300.MER [symbol] | 44 | MER76.ERVL.LTR.674.[392] | 2.98E−04 |
| 231 | K.repcat.e&g.2008.body.300.M [symbol] | 138 | MER11C.ERVK.LTR.825.[998] | 3.24E−04 |
| 232 | K.repcat.e&g.2008.body.300.M [symbol] | 29 | MER41-int.ERV1.LTR.385.[552] | 3.24E−04 |
| 233 | K.repcat.e.2008.body.300.L1P [symbol] | 349 | L1PA8.L1.LINE.7715.[856] | 3.24E−04 |
| 234 | K.repcat.e.2008.body.300.MER [symbol] | 12 | MER83B.ERV1.LTR.147.[252] | 3.24E−04 |
| 235 | K.repcat.e&g.2008.body.300.L [symbol] | 80 | LTR12B.ERV1.LTR.450.[875] | 3.52E−04 |
| 236 | K.repcat.e&g.2008.body.300.L [symbol] | 25 | LTR15.ERV1.LTR.311.[376] | 3.52E−04 |
| 237 | K.repcat.e.2008.body.300.MLT [symbol] | 1160 | MLT1B.MaLR.LTR.17353.[278] | 3.52E−04 |
| 238 | K.repcat.e.2008.body.300.MST [symbol] | 218 | MSTB1.MaLR.LTR.4879.[326] | 3.52E−04 |
| 239 | K.repcat.e.2008.body.300.Tig [symbol] | 362 | Tigger2.MER2_type.DNA.5206.[ [symbol] | 3.52E−04 |
| 240 | K.repcat.e&g.2008.body.300.L [symbol] | 651 | L1MDa.L1.LINE.9760.[522] | 3.83E−04 |
| 241 | K.repcat.e&g.2008.body.300.M [symbol] | 7 | MER65B-int.ERV1.LTR.53.[457] | 3.83E−04 |
| 242 | K.repcat.e.2008.body.300.L1M [symbol] | 741 | L1MD.L1.LINE.10847.[339] | 3.83E−04 |
| 243 | K.repcat.e.2008.body.300.LTR [symbol] | 45 | LTR36.ERV1.LTR.474.[382] | 3.83E−04 |
| 244 | K.repcat.e.2008.body.300.MER [symbol] | 14065 | MER1_type | 3.83E−04 |
| 245 | K.repcat.e&g.2008.body.300.A [symbol] | 181521 | Alu.Alu.SINE.6244.[52] | 4.16E−04 |
| 246 | K.repcat.e&g.2008.body.300.A [symbol] | 1300 | AluSg1.Alu.SINE.6311.[280] | 4.16E−04 |
| 247 | K.repcat.e&g.2008.body.300.L [symbol] | 35 | L1M3b.L1.LINE.813.[836] | 4.16E−04 |
| 248 | K.repcat.e&g.2008.body.300.L [symbol] | 456 | L1M4c.L1.LINE.7787.[595] | 4.16E−04 |
| 249 | K.repcat.e&g.2008.body.300.L [symbol] | 1764 | L1ME3B.L1.LINE.27528.[351] | 4.16E−04 |
| 250 | K.repcat.e&g.2008.body.300.L [symbol] | 24 | LTR71A.ERV1.LTR.164.[385] | 4.16E−04 |
| 251 | K.repcat.e&g.2008.body.300.P [symbol] | 121 | PiggyBac | 4.16E−04 |
| 252 | K.repcat.e.2008.body.300.L1M [symbol] | 31 | L1M3d.L1.LINE.765.[649] | 4.16E−04 |
| 253 | K.repcat.e.2008.body.300.L1P [symbol] | 59 | L1PB.L1.LINE.1520.[464] | 4.16E−04 |
| 254 | K.repcat.e.2008.body.300.MER [symbol] | 41 | MER65D.ERV1.LTR.415.[277] | 4.16E−04 |
| 255 | K.repcat.e&g.2008.body.300.L [symbol] | 385 | L1MA2.L1.LINE.6911.[992] | 4.52E−04 |
| 256 | K.repcat.e.2008.body.300.ERV [symbol] | 1507 | ERVK | 4.52E−04 |
| 257 | K.repcat.e.2008.body.300.MER [symbol] | 85 | MER34B.ERV1.LTR.1289.[331] | 4.52E−04 |
| 258 | K.repcat.e&g.2008.body.300.H [symbol] | 108 | HSMAR2.Mariner.DNA.1721.[627 [symbol] | 4.91E−04 |
| 259 | K.repcat.e&g.2008.body.300.L [symbol] | 1204 | L1MB8.L1.LINE.15156.[428] | 4.91E−04 |
| 260 | K.repcat.e&g.2008.body.300.L [symbol] | 136 | L1PA12.L1.LINE.2320.[1117] | 4.91E−04 |
| 261 | K.repcat.e&g.2008.body.300.M [symbol] | 143 | MER58C.MER1_type.DNA.2066.[1 [symbol] | 4.91E−04 |
| 262 | K.repcat.e&g.2008.body.300.H [symbol] | 1 | HERVL-C1.ERVL.LTR.7.[980] | 5.32E−04 |
| 263 | K.repcat.e&g.2008.body.300.L [symbol] | 594 | L1M1.L1.LINE.10118.[932] | 5.32E−04 |
| 264 | K.repcat.e&g.2008.body.300.L [symbol] | 8 | L1M2a1.L1.LINE.142.[918] | 5.32E−04 |
| 265 | K.repcat.e&g.2008.body.300.L [symbol] | 596 | L1MB2.L1.LINE.7699.[402] | 5.32E−04 |
| 266 | K.repcat.e&g.2008.body.300.L [symbol] | 135 | LTR9B.ERV1.LTR.745.[398] | 5.32E−04 |
| 267 | K.repcat.e&g.2008.body.300.L [symbol] | 43 | L1M3c.L1.LINE.1069.[583] | 5.77E−04 |
| 268 | K.repcat.e&g.2008.body.300.L [symbol] | 135 | L1MDb.L1.LINE.1862.[510] | 5.77E−04 |
| 269 | K.repcat.e&g.2008.body.300.L [symbol] | 329 | L1MEb.L1.LINE.5722.[435] | 5.77E−04 |
| 270 | K.repcat.e&g.2008.body.300.L [symbol] | 55 | L1P4a.L1.LINE.731.[1306] | 5.77E−04 |
| 271 | K.repcat.e&g.2008.body.300.L [symbol] | 932 | L1PA2.L1.LINE.4666.[2016] | 5.77E−04 |
| 272 | K.repcat.e&g.2008.body.300.M [symbol] | 19 | MLT1A1- | 5.77E−04 |
| 273 | K.repcat.e&g.2008.body.300.M [symbol] | 34 | MSTB-int.MaLR.LTR.617.[753] | 5.77E−04 |
| 274 | K.repcat.e.2008.body.300.MLT [symbol] | 140 | MLT1J1.MaLR.LTR.3407.[217] | 5.77E−04 |
| 275 | K.repcat.e&g.2008.body.300.L [symbol] | 59 | LTR14B.ERVK.LTR.358.[464] | 6.25E−04 |
| 276 | K.repcat.e.2008.body.300.L1M [symbol] | 1347 | L1M4.L1.LINE.19504.[380] | 6.25E−04 |
| 277 | K.repcat.e.2008.body.300.L3 | 2561 | L3.CR1.LINE.47228.[197] | 6.25E−04 |
| 278 | K.repcat.e.2008.body.300.LTR [symbol] | 54 | LTR27B.ERV1.LTR.540.[461] | 6.25E−04 |
| 279 | K.repcat.e.2008.body.300.MER [symbol] | 296 | MER30.MER1_type.DNA.3898.[19 [symbol] | 6.25E−04 |
| 280 | K.repcat.e.2008.body.300.MER [symbol] | 65 | MER51B.ERV1.LTR.697.[303] | 6.25E−04 |
| 281 | K.repcat.e&g.2008.body.300.L [symbol] | 1122 | L1ME2.L1.LINE.15797.[423] | 6.76E−04 |
| 282 | K.repcat.e&g.2008.body.300.L [symbol] | 115 | L1MEa.L1.LINE.1665.[407] | 6.76E−04 |
| 283 | K.repcat.e&g.2008.body.300.M [symbol] | 292 | MER103.DNA.DNA.6812.[111] | 6.76E−04 |
| 284 | K.repcat.e&g.2008.body.300.M [symbol] | 51 | MSTA-int.MaLR.LTR.1927.[806] | 6.76E−04 |
| 285 | K.repcat.e.2008.body.300.MER [symbol] | 98 | MER4A.ERV1.LTR.1084.[386] | 6.76E−04 |
| 286 | K.repcat.e&g.2008.body.300.H [symbol] | 370 | HAL1b.L1.LINE.7227.[255] | 7.32E−04 |
| 287 | K.repcat.e&g.2008.body.300.L [symbol] | 134 | L1PA8A.L1.LINE.2210.[1386] | 7.32E−04 |
| 288 | K.repcat.e&g.2008.body.300.L [symbol] | 102 | LTR7Y.ERV1.LTR.472.[361] | 7.32E−04 |
| 289 | K.repcat.e&g.2008.body.300.M [symbol] | 46 | MER41A- | 7.32E−04 |
| 290 | K.repcat.e&g.2008.body.300.M [symbol] | 490 | MER58B.MER1_type.DNA.6800.[2 [symbol] | 7.32E−04 |
| 291 | K.repcat.e.2008.body.300.Alu [symbol] | 6 | AluYa.Alu.SINE.46.[110] | 7.32E−04 |
| 292 | K.repcat.e&g.2008.body.300.M [symbol] | 13 | MLT1B-int.MaLR.LTR.463.[631] | 7.91E−04 |
| 293 | K.repcat.e&g.2008.body.300.T [symbol] | 218 | Tigger2a.MER2_type.DNA.3206. [symbol] | 7.91E−04 |
| 294 | K.repcat.e.2008.body.300.HER [symbol] | 65 | HERVIP10F.ERV1.LTR.318.[1464 [symbol] | 7.91E−04 |
| 295 | K.repcat.e.2008.body.300.LTR [symbol] | 113 | LTR39.ERV1.LTR.1193.[393] | 7.91E−04 |
| 296 | K.repcat.e.2008.body.300.MER [symbol] | 29 | MER54B.ERVL.LTR.417.[407] | 7.91E−04 |
| 297 | K.repcat.e&g.2008.body.300.L [symbol] | 542 | L1MA3.L1.LINE.8131.[987] | 8.55E−04 |
| 298 | K.repcat.e&g.2008.body.300.L [symbol] | 68 | LTR2B.ERV1.LTR.310.[453] | 8.55E−04 |
| 299 | K.repcat.e&g.2008.body.300.L [symbol] | 14 | LTR57-int.ERVL.LTR.309.[560] | 8.55E−04 |
| 300 | K.repcat.e&g.2008.body.300.L [symbol] | 17 | LTR73.ERV1.LTR.205.[393] | 8.55E−04 |
| 301 | K.repcat.e&g.2008.body.300.L [symbol] | 4 | LTR76-int.ERV1.LTR.8.[2020] | 8.55E−04 |
| 302 | K.repcat.e&g.2008.body.300.M [symbol] | 11 | MER4C-int.ERV1.LTR.302.[703] | 8.55E−04 |
| 303 | K.repcat.e&g.2008.body.300.M [symbol] | 15 | MER57-int.ERV1.LTR.353.[460] | 8.55E−04 |
| 304 | K.repcat.e&g.2008.body.300.M [symbol] | 1 | MER67B-int.ERV1.LTR.8.[603] | 8.55E−04 |
| 305 | K.repcat.e.2008.body.300.MER [symbol] | 99 | MER65A.ERV1.LTR.1296.[314] | 8.55E−04 |
| 306 | K.repcat.e&g.2008.body.300.M [symbol] | 57 | MER44C.MER2_type.DNA.802.[49 [symbol] | 9.24E−04 |
| 307 | K.repcat.e&g.2008.body.300.M [symbol] | 13 | MST-int.MaLR.LTR.563.[425] | 9.24E−04 |
| 308 | K.repcat.e.2008.body.300.L1P [symbol] | 123 | L1PBa.L1.LINE.1915.[1205] | 9.24E−04 |
| 309 | K.repcat.e.2008.body.300.LTR [symbol] | 34 | LTR46.ERV1.LTR.231.[325] | 9.24E−04 |
| 310 | K.repcat.e&g.2008.body.300.C [symbol] | 2938 | CR1 | 9.97E−04 |
| 311 | K.repcat.e&g.2008.body.300.H [symbol] | 83 | HERVK22.ERVK.LTR.404.[1292] | 9.97E−04 |
| 312 | K.repcat.e&g.2008.body.300.L [symbol] | 34 | L1M3e.L1.LINE.981.[813] | 9.97E−04 |
| 313 | K.repcat.e&g.2008.body.300.K [symbol] | 109 | Kanga2_a.Tc2.DNA.1465.[277] | 1.08E−03 |
| 314 | K.repcat.e&g.2008.body.300.L [symbol] | 903 | L1MEd.L1.LINE.15988.[333] | 1.08E−03 |
| 315 | K.repcat.e&g.2008.body.300.M [symbol] | 301 | MER20B.MER1_type.DNA.4150.[2 [symbol] | 1.08E−03 |
| 316 | K.repcat.e&g.2008.body.300.M [symbol] | 11 | MER31A- | 1.08E−03 |
| 317 | K.repcat.e.2008.body.300.LTR [symbol] | 304 | LTR2.ERV1.LTR.847.[415] | 1.08E−03 |
| 318 | K.repcat.e&g.2008.body.300.M [symbol] | 8 | MER61C- | 1.16E−03 |
| 319 | K.repcat.e&g.2008.body.300.L [symbol] | 8 | LTR56-int.ERV1.LTR.131.[551] | 1.25E−03 |
| 320 | K.repcat.e&g.2008.body.300.M [symbol] | 11 | MER61-int.ERV1.LTR.150.[676] | 1.25E−03 |
| 321 | K.repcat.e&g.2008.body.300.M [symbol] | 4 | MER70A- | 1.25E−03 |
| 322 | K.repcat.e&g.2008.body.300.T [symbol] | 103 | Tigger4(Zombi).MER2_type.DNA | 1.25E−03 |
| 323 | K.repcat.e.2008.body.300.MER [symbol] | 18 | MER34C.ERV1.LTR.257.[221] | 1.25E−03 |
| 324 | K.repcat.e&g.2008.body.300.A [symbol] | 2364 | AluYb9.Alu.SINE.3147.[299] | 1.35E−03 |
| 325 | K.repcat.e&g.2008.body.300.H [symbol] | 46 | HERVK3.ERVK.LTR.341.[1001] | 1.35E−03 |
| 326 | K.repcat.e&g.2008.body.300.M [symbol] | 5 | MER61F- | 1.35E−03 |
| 327 | K.repcat.e.2008.body.300.LTR [symbol] | 23 | LTR38.ERV1.LTR.313.[381] | 1.35E−03 |
| 328 | K.repcat.e.2008.body.300.MER [symbol] | 14 | MER92C.ERV1.LTR.334.[159] | 1.35E−03 |
| 329 | K.repcat.e&g.2008.body.300.L [symbol] | 750 | L1MEe.L1.LINE.12492.[333] | 1.45E−03 |
| 330 | K.repcat.e&g.2008.body.300.L [symbol] | 99 | LTR37B.ERV1.LTR.1958.[226] | 1.45E−03 |
| 331 | K.repcat.e&g.2008.body.300.L [symbol] | 18 | LTR43B.ERV1.LTR.213.[318] | 1.45E−03 |
| 332 | K.repcat.e&g.2008.body.300.M [symbol] | 5 | MER51-int.ERV1.LTR.150.[526] | 1.45E−03 |
| 333 | K.repcat.e.2008.body.300.LTR [symbol] | 19 | LTR60.ERV1.LTR.250.[309] | 1.45E−03 |
| 334 | K.repcat.e.2008.body.300.L1M [symbol] | 972 | L1M.L1.LINE.11466.[335] | 1.56E−03 |
| 335 | K.repcat.e.2008.body.300.L1M [symbol] | 1060 | L1MC.L1.LINE.16426.[272] | 1.56E−03 |
| 336 | K.repcat.e.2008.body.300.MER [symbol] | 22 | MER70B.ERVL.LTR.497.[302] | 1.56E−03 |
| 337 | K.repcat.e.2008.body.300.MLT [symbol] | 61 | MLT1E1A.MaLR.LTR.1054.[326] | 1.56E−03 |
| 338 | K.repcat.e.2008.body.300.U1 | 21 | U1.snRNA.snRNA.183.[129] | 1.56E−03 |
| 339 | K.repcat.e&g.2008.body.300.L [symbol] | 2100 | L1MC4a.L1.LINE.28404.[336] | 1.68E−03 |
| 340 | K.repcat.e&g.2008.body.300.L [symbol] | 24 | LTR40c.ERVL.LTR.609.[321] | 1.68E−03 |
| 341 | K.repcat.e&g.2008.body.300.L [symbol] | 9 | LTR68.ERV1.LTR.341.[293] | 1.80E−03 |
| 342 | K.repcat.e&g.2008.body.300.M [symbol] | 66 | MER4A1- | 1.80E−03 |
| 343 | K.repcat.e&g.2008.body.300.M [symbol] | 97 | MLT2A1.ERVL.LTR.3220.[327] | 1.80E−03 |
| 344 | K.repcat.e&g.2008.body.300.t [symbol] | 6 | tRNA-Arg- | 1.80E−03 |
| 345 | K.repcat.e&g.2008.body.300.t [symbol] | 2 | tRNA-Leu- | 1.80E−03 |
| 346 | K.repcat.e.2008.body.300.MER [symbol] | 39 | MER110A.ERV1.LTR.644.[254] | 1.80E−03 |
| 347 | K.repcat.e.2008.body.300.MER [symbol] | 152 | MER68.ERVL.LTR.1892.[361] | 1.80E−03 |
| 348 | K.repcat.e.2008.body.300.MER [symbol] | 40 | MER75.PiggyBac.DNA.551.[334] | 1.80E−03 |
| 349 | K.repcat.e&g.2008.body.300.L [symbol] | 4051 | L1M5.L1.LINE.63689.[284] | 1.94E−03 |
| 350 | K.repcat.e&g.2008.body.300.L [symbol] | 1 | LTR34-int.ERV1.LTR.26.[212] | 1.94E−03 |
| 351 | K.repcat.e&g.2008.body.300.M [symbol] | 93 | MER119.MER1_type.DNA.1169.[2 [symbol] | 1.94E−03 |
| 352 | K.repcat.e&g.2008.body.300.M [symbol] | 21 | MER21A- | 1.94E−03 |
| 353 | K.repcat.e&g.2008.body.300.M [symbol] | 12 | MER21B- | 1.94E−03 |
| 354 | K.repcat.e&g.2008.body.300.M [symbol] | 146 | MER77.ERVL.LTR.2380.[340] | 1.94E−03 |
| 355 | K.repcat.e&g.2008.body.300.M [symbol] | 86 | MuDR | 1.94E−03 |
| 356 | K.repcat.e.2008.body.300.MER [symbol] | 95 | MER39B.ERV1.LTR.1166.[382] | 1.94E−03 |
| 357 | K.repcat.e&g.2008.body.300.L [symbol] | 77 | L1M3f.L1.LINE.1234.[941] | 2.08E−03 |
| 358 | K.repcat.e&g.2008.body.300.M [symbol] | 39 | MER85.PiggyBac.DNA.892.[122] | 2.08E−03 |
| 359 | K.repcat.e.2008.body.300.Cha [symbol] | 397 | Charlie1.MER1_type.DNA.4911. [symbol] | 2.23E−03 |
| 360 | K.repcat.e.2008.body.300.ERV [symbol] | 3 | ERVL-C2.ERVL.LTR.54.[807] | 2.23E−03 |
| 361 | K.repcat.e.2008.body.300.MLT [symbol] | 258 | MLT1F.MaLR.LTR.4498.[327] | 2.23E−03 |
| 362 | K.repcat.e&g.2008.body.300.C [symbol] | 80 | Charlie9.MER1_type.DNA.1582. [symbol] | 2.39E−03 |
| 363 | K.repcat.e&g.2008.body.300.E [symbol] | 22 | ERVL-B1.ERVL.LTR.439.[1011] | 2.39E−03 |
| 364 | K.repcat.e&g.2008.body.300.M [symbol] | 1582 | MER5B.MER1_type.DNA.24584.[1 [symbol] | 2.39E−03 |
| 365 | K.repcat.e&g.2008.body.300.M [symbol] | 322 | MER7A.MER2_type.DNA.5173.[28 [symbol] | 2.39E−03 |
| 366 | K.repcat.e.2008.body.300.Alu [symbol] | 19770 | AluSg.Alu.SINE.80825.[289] | 2.39E−03 |
| 367 | K.repcat.e&g.2008.body.300.C [symbol] | 290 | Charlie7.MER1_type.DNA.4721. [symbol] | 2.57E−03 |
| 368 | K.repcat.e&g.2008.body.300.L [symbol] | 8 | L1P4e.L1.LINE.221.[996] | 2.57E−03 |
| 369 | K.repcat.e&g.2008.body.300.L [symbol] | 137 | LTR7B.ERV1.LTR.861.[364] | 2.57E−03 |
| 370 | K.repcat.e&g.2008.body.300.M [symbol] | 37 | MER105.DNA.DNA.712.[145] | 2.57E−03 |
| 371 | K.repcat.e&g.2008.body.300.M [symbol] | 41 | MER69B.Tip100.DNA.478.[511] | 2.57E−03 |
| 372 | K.repcat.e&g.2008.body.300.M [symbol] | 527 | MLT1L.MaLR.LTR.11354.[209] | 2.57E−03 |
| 373 | K.repcat.e.2008.body.300.LTR [symbol] | 429 | LTR1.ERV1.LTR.1396.[612] | 2.57E−03 |
| 374 | K.repcat.e&g.2008.body.300.M [symbol] | 181 | MARNA.Mariner.DNA.3249.[181] | 2.75E−03 |
| 375 | K.repcat.e&g.2008.body.300.M [symbol] | 85 | MER6A.MER2_type.DNA.805.[428 [symbol] | 2.75E−03 |
| 376 | K.repcat.e&g.2008.body.300.M [symbol] | 255 | MER82.MER2_type.DNA.3129.[35 [symbol] | 2.75E−03 |
| 377 | K.repcat.e.2008.body.300.L1M [symbol] | 2132 | L1MC4.L1.LINE.29881.[332] | 2.75E−03 |
| 378 | K.repcat.e.2008.body.300.L1M [symbol] | 555 | L1ME3.L1.LINE.7572.[348] | 2.75E−03 |
| 379 | K.repcat.e.2008.body.300.LTR [symbol] | 30 | LTR24C.ERV1.LTR.380.[322] | 2.75E−03 |
| 380 | K.repcat.e.2008.body.300.LTR [symbol] | 27 | LTR27.ERV1.LTR.403.[558] | 2.75E−03 |
| 381 | K.repcat.e&g.2008.body.300.A [symbol] | 116 | AluJ/FRAM.Alu.SINE.1750.[74] | 2.94E−03 |
| 382 | K.repcat.e&g.2008.body.300.L [symbol] | 24 | L1M3de.L1.LINE.497.[598] | 2.94E−03 |
| 383 | K.repcat.e&g.2008.body.300.T [symbol] | 27 | THE1D- | 2.94E−03 |
| 384 | K.repcat.e.2008.body.300.tRN [symbol] | 3 | tRNA-Met_.tRNA.tRNA.39.[44] | 2.94E−03 |
| 385 | K.repcat.e&g.2008.body.300.H [symbol] | 77 | HERVK9.ERVK.LTR.723.[1250] | 3.15E−03 |
| 386 | K.repcat.e&g.2008.body.300.L [symbol] | 1523 | L1MC5.L1.LINE.20412.[260] | 3.15E−03 |
| 387 | K.repcat.e&g.2008.body.300.M [symbol] | 136 | MER2B.MER2_type.DNA.2351.[15 [symbol] | 3.15E−03 |
| 388 | K.repcat.e&g.2008.body.300.M [symbol] | 2 | MLT1E2- | 3.15E−03 |
| 389 | K.repcat.e&g.2008.body.300.T [symbol] | 1685 | Tip100 | 3.15E−03 |
| 390 | K.repcat.e.2008.body.300.MER [symbol] | 11 | MER83C.ERV1.LTR.146.[299] | 3.15E−03 |
| 391 | K.repcat.e&g.2008.body.300.L [symbol] | 291 | L1PA6.L1.LINE.5617.[1926] | 3.37E−03 |
| 392 | K.repcat.e&g.2008.body.300.L [symbol] | 653 | L1PB1.L1.LINE.11421.[1064] | 3.37E−03 |
| 393 | K.repcat.e&g.2008.body.300.M [symbol] | 33 | MER41B- | 3.37E−03 |
| 394 | K.repcat.e&g.2008.body.300.M [symbol] | 7 | MLT1F1- | 3.37E−03 |
| 395 | K.repcat.e&g.2008.body.300.L [symbol] | 23 | L1M2c.L1.LINE.585.[856] | 3.61E−03 |
| 396 | K.repcat.e&g.2008.body.300.M [symbol] | 63 | MLT2C2.ERVL.LTR.1282.[249] | 3.61E−03 |
| 397 | K.repcat.e.2008.body.300.MST [symbol] | 62 | MSTB2.MaLR.LTR.1595.[329] | 3.61E−03 |
| 398 | K.repcat.e&g.2008.body.300.B [symbol] | 104 | BLACKJACK.AcHobo.DNA.2099.[3 [symbol] | 3.85E−03 |
| 399 | K.repcat.e&g.2008.body.300.H [symbol] | 42 | HUERS-P1.ERV1.LTR.592.[986] | 3.85E−03 |
| 400 | K.repcat.e&g.2008.body.300.L [symbol] | 23 | LOR1b-int.ERV1.LTR.354.[624] | 3.85E−03 |
| 401 | K.repcat.e.2008.body.300.MER [symbol] | 125 | MER49.ERV1.LTR.1296.[399] | 3.85E−03 |
| 402 | K.repcat.e.2008.body.300.MER [symbol] | 63 | MER52D.ERV1.LTR.576.[932] | 3.85E−03 |
| 403 | K.repcat.e.2008.body.300.MER [symbol] | 17 | MER61E.ERV1.LTR.308.[387] | 3.85E−03 |
| 404 | K.repcat.e&g.2008.body.300.M [symbol] | 1 | MER41E-int.ERV1.LTR.14.[528] | 4.12E−03 |
| 405 | K.repcat.e&g.2008.body.300.M [symbol] | 117 | MER44A.MER2_type.DNA.2119.[2 [symbol] | 4.39E−03 |
| 406 | K.repcat.e&g.2008.body.300.P [symbol] | 21 | PABL_A- | 4.39E−03 |
| 407 | K.repcat.e.2008.body.300.HAL [symbol] | 1862 | HAL1.L1.LINE.27468.[342] | 4.39E−03 |
| 408 | K.repcat.e&g.2008.body.300.A [symbol] | 3246 | AluSg/x.Alu.SINE.25834.[162] | 4.69E−03 |
| 409 | K.repcat.e&g.2008.body.300.M [symbol] | 39 | MER101- | 4.69E−03 |
| 410 | K.repcat.e&g.2008.body.300.M [symbol] | 4 | MER4D1-int.ERV1.LTR.53.[619] | 4.69E−03 |
| 411 | K.repcat.e.2008.body.300.LTR [symbol] | 42 | LTR26.ERV1.LTR.604.[405] | 4.69E−03 |
| 412 | K.repcat.e&g.2008.body.300.L [symbol] | 17 | L1P4d.L1.LINE.204.[1102] | 5.00E−03 |
| 413 | K.repcat.e.2008.body.300.Alu [symbol] | 3067 | AluYa5.Alu.SINE.3978.[292] | 5.00E−03 |
| 414 | K.repcat.e.2008.body.300.MER [symbol] | 714 | MER3.MER1_type.DNA.10996.[14 [symbol] | 5.00E−03 |
| 415 | K.repcat.e.2008.body.300.tRN [symbol] | 1 | tRNA-Met.tRNA.tRNA.48.[57] | 5.00E−03 |
| 416 | K.repcat.e&g.2008.body.300.L [symbol] | 7 | L1P4c.L1.LINE.93.[937] | 5.33E−03 |
| 417 | K.repcat.e&g.2008.body.300.L [symbol] | 4 | LTR24-int.ERV1.LTR.107.[555] | 5.33E−03 |
| 418 | K.repcat.e&g.2008.body.300.M [symbol] | 36 | MER50-int.ERV1.LTR.488.[791] | 5.33E−03 |
| 419 | K.repcat.e&g.2008.body.300.M [symbol] | 26 | MER6B.MER2_type.DNA.656.[170 [symbol] | 5.33E−03 |
| 420 | K.repcat.e.2008.body.300.Alu [symbol] | 15732 | AluSq.Alu.SINE.93297.[286] | 5.33E−03 |
| 421 | K.repcat.e.2008.body.300.MER [symbol] | 350 | MER21C.ERV1.LTR.4871.[411] | 5.33E−03 |
| 422 | K.repcat.e&g.2008.body.300.C [symbol] | 22 | Charlie6.MER1_type.DNA.250.[ [symbol] | 5.68E−03 |
| 423 | K.repcat.e&g.2008.body.300.M [symbol] | 59 | MER54A.ERVL.LTR.626.[458] | 5.68E−03 |
| 424 | K.repcat.e.2008.body.300.Alu [symbol] | 13437 | AluSp.Alu.SINE.50469.[287] | 5.68E−03 |
| 425 | K.repcat.e&g.2008.body.300.L [symbol] | 19 | LTR72B.ERV1.LTR.176.[304] | 6.05E−03 |
| 426 | K.repcat.e&g.2008.body.300.M [symbol] | 299 | MER1A.MER1_type.DNA.2968.[45 [symbol] | 6.05E−03 |
| 427 | K.repcat.e&g.2008.body.300.M [symbol] | 8 | MER61D- | 6.05E−03 |
| 428 | K.repcat.e&g.2008.body.300.L [symbol] | 12 | L1M2b.L1.LINE.318.[858] | 6.44E−03 |
| 429 | K.repcat.e&g.2008.body.300.L [symbol] | 93 | LTR10F.ERV1.LTR.432.[415] | 6.44E−03 |
| 430 | K.repcat.e&g.2008.body.300.L [symbol] | 41 | LTR53.ERVL.LTR.666.[362] | 6.44E−03 |
| 431 | K.repcat.e&g.2008.body.300.R [symbol] | 8 | Ricksha_c.MuDR.DNA.121.[854] | 6.44E−03 |
| 432 | K.repcat.e&g.2008.body.300.L [symbol] | 462 | LTR16C.ERVL.LTR.9063.[251] | 6.86E−03 |
| 433 | K.repcat.e&g.2008.body.300.L [symbol] | 143 | LTR50.ERVL.LTR.2809.[267] | 6.86E−03 |
| 434 | K.repcat.e&g.2008.body.300.M [symbol] | 20 | MSTC-int.MaLR.LTR.193.[799] | 6.86E−03 |
| 435 | K.repcat.e&g.2008.body.300.C [symbol] | 66 | Cheshire.MER1_type.DNA.747.[ [symbol] | 7.29E−03 |
| 436 | K.repcat.e&g.2008.body.300.L [symbol] | 48 | L1PBA1.L1.LINE.656.[1545] | 7.29E−03 |
| 437 | K.repcat.e&g.2008.body.300.M [symbol] | 178 | MER46A.MER2_type.DNA.3411.[1 [symbol] | 7.29E−03 |
| 438 | K.repcat.e&g.2008.body.300.L [symbol] | 1775 | L1MB7.L1.LINE.20238.[456] | 7.75E−03 |
| 439 | K.repcat.e&g.2008.body.300.M [symbol] | 2 | MER67C-int.ERV1.LTR.24.[346] | 7.75E−03 |
| 440 | K.repcat.e&g.2008.body.300.t [symbol] | 3 | tRNA-Thr- | 7.75E−03 |
| 441 | K.repcat.e.2008.body.300.HER [symbol] | 52 | HERVK14.ERVK.LTR.297.[755] | 7.75E−03 |
| 442 | K.repcat.e&g.2008.body.300.M [symbol] | 15 | MER21C- | 8.24E−03 |
| 443 | K.repcat.e&g.2008.body.300.M [symbol] | 139 | MLT-int.MaLR.LTR.4461.[1279] | 8.24E−03 |
| 444 | K.repcat.e&g.2008.body.300.T [symbol] | 428 | Tc2 | 8.24E−03 |
| 445 | K.repcat.e.2008.body.300.PAB [symbol] | 47 | PABL_A.ERV1.LTR.514.[471] | 8.24E−03 |
| 446 | K.repcat.e&g.2008.body.300.M [symbol] | 632 | MER1B.MER1_type.DNA.5233.[29 [symbol] | 8.75E−03 |
| 447 | K.repcat.e&g.2008.body.300.M [symbol] | 9 | MER65C- | 8.75E−03 |
| 448 | K.repcat.e&g.2008.body.300.M [symbol] | 5 | MER72-int.ERV1.LTR.69.[429] | 8.75E−03 |
| 449 | K.repcat.e&g.2008.body.300.T [symbol] | 54 | Tigger6a.MER2_type.DNA.681.[ [symbol] | 8.75E−03 |
| 450 | K.repcat.e&g.2008.body.300.t [symbol] | 10 | tRNA-Val- | 8.75E−03 |
| 451 | K.repcat.e.2008.body.300.LTR [symbol] | 16 | LTR10G.ERV1.LTR.116.[432] | 8.75E−03 |
| 452 | K.repcat.e.2008.body.300.LTR [symbol] | 42 | LTR30.ERV1.LTR.170.[560] | 8.75E−03 |
| 453 | K.repcat.e.2008.body.300.MER [symbol] | 51 | MER72.ERV1.LTR.736.[435] | 8.75E−03 |
| 454 | K.repcat.e.2008.body.300.LTR [symbol] | 61 | LTR40b.ERVL.LTR.1229.[270] | 9.29E−03 |
| 455 | K.repcat.e.2008.body.300.MER [symbol] | 118 | MER8.MER2_type.DNA.1884.[188 [symbol] | 9.29E−03 |
| 456 | K.repcat.e&g.2008.body.300.M [symbol] | 6 | MER4A1_- | 9.86E−03 |
| 457 | K.repcat.e&g.2008.body.300.t [symbol] | 7 | tRNA-Val- | 9.86E−03 |
| 458 | K.repcat.e.2008.body.300.LOR [symbol] | 22 | LOR1a.ERV1.LTR.789.[380] | 9.86E−03 |
| 459 | K.repcat.e&g.2008.body.300.L [symbol] | 37 | LTR3A.ERVK.LTR.158.[396] | 1.05E−02 |
| 460 | K.repcat.e&g.2008.body.300.M [symbol] | 9 | MER4E1-int.ERV1.LTR.75.[590] | 1.05E−02 |
| 461 | K.repcat.e&g.2008.body.300.U [symbol] | 127 | U6.snRNA.snRNA.1666.[87] | 1.05E−02 |
| 462 | K.repcat.e.2008.body.300.LTR [symbol] | 486 | LTR33.ERVL.LTR.9302.[253] | 1.05E−02 |
| 463 | K.repcat.e.2008.body.300.Ric [symbol] | 64 | Ricksha.MuDR.DNA.1551.[287] | 1.05E−02 |
| 464 | K.repcat.e&g.2008.body.300.L [symbol] | 308 | LTR12D.ERV1.LTR.502.[810] | 1.11E−02 |
| 465 | K.repcat.e&g.2008.body.300.L [symbol] | 13 | LTR38C.ERV1.LTR.132.[373] | 1.11E−02 |
| 466 | K.repcat.e&g.2008.body.300.M [symbol] | 4 | MER52A- | 1.11E−02 |
| 467 | K.repcat.e&g.2008.body.300.M [symbol] | 12 | MER92A.ERV1.LTR.145.[271] | 1.11E−02 |
| 468 | K.repcat.e&g.2008.body.300.T [symbol] | 159 | Tigger5a.MER2_type.DNA.2545. [symbol] | 1.11E−02 |
| 469 | K.repcat.e.2008.body.300.MER [symbol] | 500 | MER2.MER2_type.DNA.8174.[238 [symbol] | 1.11E−02 |
| 470 | K.repcat.e&g.2008.body.300.L [symbol] | 2335 | L1ME4a.L1.LINE.38237.[216] | 1.18E−02 |
| 471 | K.repcat.e&g.2008.body.300.S [symbol] | 126 | SST1.centr.Satellite.565.[60 [symbol] | 1.18E−02 |
| 472 | K.repcat.e&g.2008.body.300.T [symbol] | 25 | THE1A-int.MaLR.LTR.615.[948] | 1.18E−02 |
| 473 | K.repcat.e&g.2008.body.300.M [symbol] | 17 | MER41G.ERV1.LTR.105.[415] | 1.24E−02 |
| 474 | K.repcat.e&g.2008.body.300.M [symbol] | 4 | MER75B.PiggyBac.DNA.111.[168 [symbol] | 1.24E−02 |
| 475 | K.repcat.e.2008.body.300.MER [symbol] | 35 | MER41E.ERV1.LTR.355.[382] | 1.24E−02 |
| 476 | K.repcat.e&g.2008.body.300.E [symbol] | 20 | ERVL-F.ERVL.LTR.459.[601] | 1.32E−02 |
| 477 | K.repcat.e&g.2008.body.300.H [symbol] | 38 | HERVP71A.ERV1.LTR.190.[1344] | 1.32E−02 |
| 478 | K.repcat.e&g.2008.body.300.L [symbol] | 19 | LTR14A.ERVK.LTR.113.[329] | 1.32E−02 |
| 479 | K.repcat.e&g.2008.body.300.M [symbol] | 222 | MER112.MER1_type.DNA.3729.[1 [symbol] | 1.32E−02 |
| 480 | K.repcat.e&g.2008.body.300.M [symbol] | 30 | MER44D.MER2_type.DNA.334.[49 [symbol] | 1.32E−02 |
| 481 | K.repcat.e.2008.body.300.LTR [symbol] | 79 | LTR54.ERV1.LTR.1087.[261] | 1.32E−02 |
| 482 | K.repcat.e&g.2008.body.300.A [symbol] | 45649 | AluSx.Alu.SINE.332026.[290] | 1.39E−02 |
| 483 | K.repcat.e&g.2008.body.300.H [symbol] | 35 | HERV35I.ERV1.LTR.426.[594] | 1.39E−02 |
| 484 | K.repcat.e&g.2008.body.300.M [symbol] | 406 | MER1_type? | 1.39E−02 |
| 485 | K.repcat.e&g.2008.body.300.M [symbol] | 38 | MLT1D-int.MaLR.LTR.595.[731] | 1.39E−02 |
| 486 | K.repcat.e.2008.body.300.LTR [symbol] | 19 | LTR38B.ERV1.LTR.217.[386] | 1.39E−02 |
| 487 | K.repcat.e.2008.body.300.MER [symbol] | 265 | MER21B.ERV1.LTR.3565.[442] | 1.39E−02 |
| 488 | K.repcat.e&g.2008.body.300.L [symbol] | 13 | LTR25-int.ERV1.LTR.264.[950] | 1.48E−02 |
| 489 | K.repcat.e&g.2008.body.300.M [symbol] | 134 | MER46B.MER2_type.DNA.2000.[2 [symbol] | 1.48E−02 |
| 490 | K.repcat.e&g.2008.body.300.M [symbol] | 5 | MLT1E-int.MaLR.LTR.109.[760] | 1.48E−02 |
| 491 | K.repcat.e.2008.body.300.MER [symbol] | 30 | MER4D0.ERV1.LTR.463.[369] | 1.48E−02 |
| 492 | K.repcat.e.2008.body.300.tRN [symbol] | 2 | tRNA-Leu- | 1.48E−02 |
| 493 | K.repcat.e&g.2008.body.300.A [symbol] | 562 | AluYd8.Alu.SINE.853.[274] | 1.56E−02 |
| 494 | K.repcat.e&g.2008.body.300.M [symbol] | 30 | MER65-int.ERV1.LTR.303.[493] | 1.56E−02 |
| 495 | K.repcat.e.2008.body.300.MER [symbol] | 93 | MER4C.ERV1.LTR.1172.[320] | 1.56E−02 |
| 496 | K.repcat.e&g.2008.body.300.L [symbol] | 14 | L1P3b.L1.LINE.165.[1351] | 1.65E−02 |
| 497 | K.repcat.e&g.2008.body.300.L [symbol] | 9 | LTR43-int.ERV1.LTR.97.[655] | 1.65E−02 |
| 498 | K.repcat.e&g.2008.body.300.M [symbol] | 9 | MER76-int.ERVL.LTR.155.[543] | 1.65E−02 |
| 499 | K.repcat.e.2008.body.300.LTR [symbol] | 14 | LTR76.ERV1.LTR.186.[480] | 1.65E−02 |
| 500 | K.repcat.e&g.2008.body.300.H [symbol] | 129 | Harlequin.ERV1.LTR.610.[1500 [symbol] | 1.74E−02 |
| 501 | K.repcat.e&g.2008.body.300.H [symbol] | 13 | HERV30.ERV1.LTR.61.[1647] | 1.74E−02 |
| 502 | K.repcat.e&g.2008.body.300.L [symbol] | 17 | L1P4b.L1.LINE.275.[1316] | 1.74E−02 |
| 503 | K.repcat.e.2008.body.300.MER [symbol] | 58 | MER65C.ERV1.LTR.683.[311] | 1.74E−02 |
| 504 | K.repcat.e.2008.body.300.MLT [symbol] | 1583 | MLT1C.MaLR.LTR.19102.[313] | 1.74E−02 |
| 505 | K.repcat.e&g.2008.body.300.M [symbol] | 15 | MLT1-int.MaLR.LTR.283.[323] | 1.84E−02 |
| 506 | K.repcat.e&g.2008.body.300.t [symbol] | 2 | tRNA-Thr- | 1.84E−02 |
| 507 | K.repcat.e&g.2008.body.300.L [symbol] | 30 | LTR14C.ERVK.LTR.138.[512] | 1.94E−02 |
| 508 | K.repcat.e.2008.body.300.LTR [symbol] | 75 | LTR56.ERV1.LTR.939.[275] | 1.94E−02 |
| 509 | K.repcat.e&g.2008.body.300.C [symbol] | 337 | Charlie1a.MER1_type.DNA.3608 [symbol] | 2.05E−02 |
| 510 | K.repcat.e&g.2008.body.300.H [symbol] | 43 | HY1.scRNA.scRNA.428.[97] | 2.05E−02 |
| 511 | K.repcat.e.2008.body.300.ERV [symbol] | 13 | ERVL-B3.ERVL.LTR.194.[1101] | 2.05E−02 |
| 512 | K.repcat.e.2008.body.300.FAM [symbol] | 307 | FAM.Alu.SINE.4424.[149] | 2.05E−02 |
| 513 | K.repcat.e&g.2008.body.300.A [symbol] | 40 | ALR/Alpha.centr.Satellite.11 [symbol] | 2.16E−02 |
| 514 | K.repcat.e&g.2008.body.300.L [symbol] | 80 | LTR49- | 2.16E−02 |
| 515 | K.repcat.e&g.2008.body.300.M [symbol] | 119 | MADE2.Mariner.DNA.2609.[69] | 2.16E−02 |
| 516 | K.repcat.e&g.2008.body.300.M [symbol] | 8 | MLT1G-int.MaLR.LTR.121.[679] | 2.16E−02 |
| 517 | K.repcat.e&g.2008.body.300.T [symbol] | 21 | THE1C- | 2.16E−02 |
| 518 | K.repcat.e.2008.body.300.Cha [symbol] | 246 | Charlie2.MER1_type.DNA.3300. [symbol] | 2.16E−02 |
| 519 | K.repcat.e&g.2008.body.300.A [symbol] | 56 | AluYh9.Alu.SINE.183.[159] | 2.28E−02 |
| 520 | K.repcat.e&g.2008.body.300.L [symbol] | 5 | LTR12C-int.ERV1.LTR.26.[561] | 2.28E−02 |
| 521 | K.repcat.e&g.2008.body.300.M [symbol] | 11 | MER110- | 2.28E−02 |
| 522 | K.repcat.e.2008.body.300.MER [symbol] | 152 | MER58.MER1_type.DNA.2330.[10 [symbol] | 2.28E−02 |
| 523 | K.repcat.e&g.2008.body.300.C [symbol] | 113 | Charlie2a.MER1_type.DNA.1648 [symbol] | 2.40E−02 |
| 524 | K.repcat.e.2008.body.300.LTR [symbol] | 23 | LTR19B.ERV1.LTR.277.[423] | 2.40E−02 |
| 525 | K.repcat.e.2008.body.300.MER [symbol] | 37 | MER84.ERV1.LTR.345.[286] | 2.40E−02 |
| 526 | K.repcat.e&g.2008.body.300.L [symbol] | 22 | LTR39-int.ERV1.LTR.295.[362] | 2.53E−02 |
| 527 | K.repcat.e&g.2008.body.300.M [symbol] | 13 | MER107.MER1_type.DNA.320.[15 [symbol] | 2.53E−02 |
| 528 | K.repcat.e&g.2008.body.300.M [symbol] | 186 | MER46C.MER2_type.DNA.2705.[2 [symbol] | 2.53E−02 |
| 529 | K.repcat.e.2008.body.300.Kan [symbol] | 175 | Kanga1.Tc2.DNA.3485.[171] | 2.53E−02 |
| 530 | K.repcat.e.2008.body.300.LTR [symbol] | 53 | LTR48B.ERV1.LTR.1058.[346] | 2.53E−02 |
| 531 | K.repcat.e&g.2008.body.300.M [symbol] | 67 | MER4-int.ERV1.LTR.918.[310] | 2.66E−02 |
| 532 | K.repcat.e&g.2008.body.300.M [symbol] | 9 | MER93B-int | 2.66E−02 |
| 533 | K.repcat.e&g.2008.body.300.t [symbol] | 6 | tRNA-Lys- | 2.66E−02 |
| 534 | K.repcat.e.2008.body.300.Alu [symbol] | 34 | AluYb.Alu.SINE.151.[123] | 2.66E−02 |
| 535 | K.repcat.e.2008.body.300.LTR [symbol] | 342 | LTR17.ERV1.LTR.830.[578] | 2.66E−02 |
| 536 | K.repcat.e.2008.body.300.LTR [symbol] | 12 | LTR3B.ERVK.LTR.90.[391] | 2.66E−02 |
| 537 | K.repcat.e&g.2008.body.300.A [symbol] | 56 | AluYc5.Alu.SINE.496.[51] | 2.80E−02 |
| 538 | K.repcat.e&g.2008.body.300.L [symbol] | 32 | LTR1C.ERV1.LTR.242.[459] | 2.80E−02 |
| 539 | K.repcat.e&g.2008.body.300.M [symbol] | 7 | MER95.ERV.LTR.154.[264] | 2.80E−02 |
| 540 | K.repcat.e&g.2008.body.300.M [symbol] | 11 | MER9B.ERVK.LTR.61.[431] | 2.80E−02 |
| 541 | K.repcat.e&g.2008.body.300.t [symbol] | 6 | tRNA-Ser- | 2.80E−02 |
| 542 | K.repcat.e&g.2008.body.300.R [symbol] | 4 | Ricksha_0.MuDR.DNA.99.[617] | 2.95E−02 |
| 543 | K.repcat.e.2008.body.300.Alu [symbol] | 287 | AluS.Alu.SINE.2733.[76] | 2.95E−02 |
| 544 | K.repcat.e&g.2008.body.300.B [symbol] | 48 | BSR/Beta.Satellite.Satellite [symbol] | 3.10E−02 |
| 545 | K.repcat.e&g.2008.body.300.M [symbol] | 56 | MER63C.AcHobo.DNA.602.[467] | 3.10E−02 |
| 546 | K.repcat.e&g.2008.body.300.L [symbol] | 53 | LTR64.ERV1.LTR.352.[292] | 3.26E−02 |
| 547 | K.repcat.e&g.2008.body.300.M [symbol] | 49 | MER7C.MER2_type.DNA.588.[397 [symbol] | 3.26E−02 |
| 548 | K.repcat.e&g.2008.body.300.t [symbol] | 7 | tRNA-Ser- | 3.26E−02 |
| 549 | K.repcat.e&g.2008.body.300.t [symbol] | 4 | tRNA-Thr- | 3.26E−02 |
| 550 | K.repcat.e.2008.body.300.MER [symbol] | 203 | MER52A.ERV1.LTR.1824.[1041] | 3.26E−02 |
| 551 | K.repcat.e&g.2008.body.300.C [symbol] | 261 | Charlie5.MER1_type.DNA.3466. [symbol] | 3.43E−02 |
| 552 | K.repcat.e&g.2008.body.300.K [symbol] | 14 | Kanga1c.Tc2.DNA.186.[374] | 3.43E−02 |
| 553 | K.repcat.e&g.2008.body.300.M [symbol] | 9 | MER4E-int.ERV1.LTR.151.[712] | 3.43E−02 |
| 554 | K.repcat.e&g.2008.body.300.P [symbol] | 160 | PRIMA4- | 3.43E−02 |
| 555 | K.repcat.e.2008.body.300.HER [symbol] | 778 | HERVH.ERV1.LTR.5774.[958] | 3.43E−02 |
| 556 | K.repcat.e.2008.body.300.MER [symbol] | 51 | MER4E.ERV1.LTR.694.[476] | 3.43E−02 |
| 557 | K.repcat.e.2008.body.300.MER [symbol] | 28 | MER87.ERV1.LTR.405.[280] | 3.43E−02 |
| 558 | K.repcat.e&g.2008.body.300.L [symbol] | 134 | LTR5_Hs.ERVK.LTR.601.[880] | 3.60E−02 |
| 559 | K.repcat.e&g.2008.body.300.M [symbol] | 1 | MER34C_- | 3.60E−02 |
| 560 | K.repcat.e&g.2008.body.300.M [symbol] | 3 | MLT1E1-int.MaLR.LTR.56.[576] | 3.60E−02 |
| 561 | K.repcat.e&g.2008.body.300.t [symbol] | 1 | tRNA-Ala- | 3.60E−02 |
| 562 | K.repcat.e.2008.body.300.MLT [symbol] | 207 | MLT1H2.MaLR.LTR.4227.[216] | 3.60E−02 |
| 563 | K.repcat.e.2008.body.300.PAB [symbol] | 26 | PABL_B.ERV1.LTR.361.[373] | 3.60E−02 |
| 564 | K.repcat.e&g.2008.body.300.A [symbol] | 180 | AluYc3.Alu.SINE.1758.[58] | 3.78E−02 |
| 565 | K.repcat.e&g.2008.body.300.H [symbol] | 13 | HERVK14C.ERVK.LTR.81.[1701] | 3.78E−02 |
| 566 | K.repcat.e&g.2008.body.300.S [symbol] | 60 | SATR2.Satellite.Satellite.25 [symbol] | 3.78E−02 |
| 567 | K.repcat.e.2008.body.300.tRN [symbol] | 4 | tRNA-Arg- | 3.78E−02 |
| 568 | K.repcat.e&g.2008.body.300.7 [symbol] | 58 | 7SK.RNA.RNA.715.[165] | 3.97E−02 |
| 569 | K.repcat.e&g.2008.body.300.H [symbol] | 148 | HERV9.ERV1.LTR.1084.[1506] | 3.97E−02 |
| 570 | K.repcat.e&g.2008.body.300.M [symbol] | 102 | MER5C.MER1_type.DNA.1512.[16 [symbol] | 3.97E−02 |
| 571 | K.repcat.e&g.2008.body.300.M [symbol] | 2 | MLT1E1A- | 3.97E−02 |
| 572 | K.repcat.e&g.2008.body.300.T [symbol] | 71 | Tigger5b.MER2_type.DNA.775.[ [symbol] | 3.97E−02 |
| 573 | K.repcat.e&g.2008.body.300.t [symbol] | 9 | tRNA-Asp- | 3.97E−02 |
| 574 | K.repcat.e.2008.body.300.MER [symbol] | 40 | MER91.Tip100.DNA.553.[55] | 3.97E−02 |
| 575 | K.repcat.e.2008.body.300.RNA [symbol] | 58 | RNA | 3.97E−02 |
| 576 | K.repcat.e&g.2008.body.300.L [symbol] | 38 | Looper.PiggyBac.DNA.514.[348 [symbol] | 4.16E−02 |
| 577 | K.repcat.e&g.2008.body.300.L [symbol] | 1 | LTR10C-int.ERV1.LTR.11.[163] | 4.16E−02 |
| 578 | K.repcat.e.2008.body.300.LTR [symbol] | 46 | LTR43.ERV1.LTR.436.[329] | 4.16E−02 |
| 579 | K.repcat.e.2008.body.300.MER [symbol] | 18 | MER65B.ERV1.LTR.238.[194] | 4.16E−02 |
| 580 | K.repcat.e.2008.body.300.tRN [symbol] | 1 | tRNA-Gln- | 4.16E−02 |
| 581 | K.repcat.e&g.2008.body.300.M [symbol] | 1 | MER51E-int.ERV1.LTR.25.[430] | 4.37E−02 |
| 582 | K.repcat.e&g.2008.body.300.M [symbol] | 70 | MER63D.AcHobo.DNA.1023.[205] | 4.37E−02 |
| 583 | K.repcat.e&g.2008.body.300.M [symbol] | 80 | MER74B.ERVL.LTR.936.[328] | 4.37E−02 |
| 584 | K.repcat.e.2008.body.300.MLT [symbol] | 1024 | MLT1K.MaLR.LTR.18519.[227] | 4.37E−02 |
| 585 | K.repcat.e&g.2008.body.300.M [symbol] | 5 | MER83-int.ERV1.LTR.63.[852] | 4.58E−02 |
| 586 | K.repcat.e&g.2008.body.300.M [symbol] | 14 | MER101B.ERV1.LTR.239.[304] | 4.80E−02 |
| 587 | K.repcat.e&g.2008.body.300.s [symbol] | 129 | scRNA | 4.80E−02 |
| 588 | K.repcat.e.2008.body.300.Alu [symbol] | 219 | AluJ.Alu.SINE.2554.[134] | 4.80E−02 |
| 589 | K.repcat.e&g.2008.body.300.H [symbol] | 7 | HSAT4.centr.Satellite.96.[24 [symbol] | 5.03E−02 |
| 590 | K.repcat.e&g.2008.body.300.O [symbol] | 101 | ORSL.Tip100.DNA.1181.[165] | 5.03E−02 |
| 591 | K.repcat.e.2008.body.300.LTR [symbol] | 68 | LTR48.ERV1.LTR.1227.[342] | 5.03E−02 |
| 592 | K.repcat.e&g.2008.body.300.A [symbol] | 795 | AluSq/x.Alu.SINE.7043.[122] | 5.26E−02 |
| 593 | K.repcat.e&g.2008.body.300.H [symbol] | 3 | HSATI.Satellite.Satellite.43 [symbol] | 5.26E−02 |
| 594 | K.repcat.e&g.2008.body.300.L [symbol] | 1556 | L1MB3.L1.LINE.15139.[387] | 5.26E−02 |
| 595 | K.repcat.e&g.2008.body.300.M [symbol] | 45 | MER65A- | 5.26E−02 |
| 596 | K.repcat.e.2008.body.300.LTR [symbol] | 16 | LTR57.ERVL.LTR.234.[327] | 5.26E−02 |
| 597 | K.repcat.e&g.2008.body.300.A [symbol] | 33 | AluYa5/8.Alu.SINE.112.[108] | 5.51E−02 |
| 598 | K.repcat.e&g.2008.body.300.H [symbol] | 13 | HERVKC4.ERVK.LTR.63.[885] | 5.51E−02 |
| 599 | K.repcat.e&g.2008.body.300.L [symbol] | 30 | LTR18A.ERVL.LTR.250.[313] | 5.51E−02 |
| 600 | K.repcat.e&g.2008.body.300.M [symbol] | 7 | MER39B-int.ERV1.LTR.62.[638] | 5.51E−02 |
| 601 | K.repcat.e&g.2008.body.300.M [symbol] | 1 | Merlin | 5.51E−02 |
| 602 | K.repcat.e&g.2008.body.300.M [symbol] | 1 | Merlin1_HS.Merlin.DNA.53.[33 [symbol] | 5.51E−02 |
| 603 | K.repcat.e&g.2008.body.300.L [symbol] | 3 | LTR48B- | 5.76E−02 |
| 604 | K.repcat.e&g.2008.body.300.M [symbol] | 3 | MER6C.MER2_type.DNA.136.[148 [symbol] | 5.76E−02 |
| 605 | K.repcat.e.2008.body.300.FRA [symbol] | 1620 | FRAM.Alu.SINE.21803.[149] | 5.76E−02 |
| 606 | K.repcat.e.2008.body.300.MER [symbol] | 131 | MER66B.ERV1.LTR.1407.[302] | 5.76E−02 |
| 607 | K.repcat.e&g.2008.body.300.M [symbol] | 3 | MER87-int.ERV1.LTR.25.[449] | 6.03E−02 |
| 608 | K.repcat.e&g.2008.body.300.t [symbol] | 7 | tRNA-Gly- | 6.03E−02 |
| 609 | K.repcat.e.2008.body.300.MLT [symbol] | 152 | MLT1G.MaLR.LTR.3459.[318] | 6.03E−02 |
| 610 | K.repcat.e.2008.body.300.tRN [symbol] | 4 | tRNA-Glu- | 6.03E−02 |
| 611 | K.repcat.e&g.2008.body.300.M [symbol] | 26 | MER68-int.ERVL.LTR.344.[465] | 6.30E−02 |
| 612 | K.repcat.e&g.2008.body.300.A [symbol] | 52 | AluYa8.Alu.SINE.107.[226] | 6.59E−02 |
| 613 | K.repcat.e&g.2008.body.300.M [symbol] | 7 | MER84-int.ERV1.LTR.174.[637] | 6.59E−02 |
| 614 | K.repcat.e&g.2008.body.300.M [symbol] | 15 | MLT1G1- | 6.59E−02 |
| 615 | K.repcat.e.2008.body.300.HER [symbol] | 30 | HERVI.ERV1.LTR.124.[2141] | 6.59E−02 |
| 616 | K.repcat.e&g.2008.body.300.H [symbol] | 107 | HERV17.ERV1.LTR.919.[982] | 6.88E−02 |
| 617 | K.repcat.e&g.2008.body.300.M [symbol] | 1 | MER52C-int.ERV1.LTR.67.[636] | 6.88E−02 |
| 618 | K.repcat.e&g.2008.body.300.U [symbol] | 13 | U3.snRNA.snRNA.228.[102] | 6.88E−02 |
| 619 | K.repcat.e.2008.body.300.LTR [symbol] | 10 | LTR59.ERV1.LTR.121.[337] | 6.88E−02 |
| 620 | K.repcat.e.2008.body.300.MER [symbol] | 38 | MER61B.ERV1.LTR.409.[332] | 7.19E−02 |
| 621 | K.repcat.e.2008.body.300.MER [symbol] | 41 | MER66A.ERV1.LTR.346.[353] | 7.19E−02 |
| 622 | K.repcat.e&g.2008.body.300.L [symbol] | 8 | LTR48-int.ERV1.LTR.107.[433] | 7.50E−02 |
| 623 | K.repcat.e.2008.body.300.LTR [symbol] | 33 | LTR45B.ERV1.LTR.417.[324] | 7.50E−02 |
| 624 | K.repcat.e&g.2008.body.300.a [symbol] | 12 | acro | 7.83E−02 |
| 625 | K.repcat.e&g.2008.body.300.A [symbol] | 12 | ACRO1.acro.Satellite.40.[730 [symbol] | 7.83E−02 |
| 626 | K.repcat.e&g.2008.body.300.H [symbol] | 25 | HERVL74.ERVL.LTR.473.[425] | 7.83E−02 |
| 627 | K.repcat.e&g.2008.body.300.L [symbol] | 56 | LTR32.ERVL.LTR.1289.[281] | 7.83E−02 |
| 628 | K.repcat.e&g.2008.body.300.L [symbol] | 16 | LTR23-int.ERV1.LTR.219.[363] | 8.17E−02 |
| 629 | K.repcat.e&g.2008.body.300.L [symbol] | 3 | LTR27-int.ERV1.LTR.76.[1024] | 8.17E−02 |
| 630 | K.repcat.e&g.2008.body.300.M [symbol] | 25 | MER89-int.ERV1.LTR.614.[493] | 8.17E−02 |
| 631 | K.repcat.e&g.2008.body.300.L [symbol] | 2 | LTR1D-int.ERV1.LTR.40.[2150] | 8.52E−02 |
| 632 | K.repcat.e&g.2008.body.300.L [symbol] | 34 | LTR45C.ERV1.LTR.393.[324] | 8.52E−02 |
| 633 | K.repcat.e&g.2008.body.300.U [symbol] | 4 | U17.snRNA.snRNA.9.[174] | 8.52E−02 |
| 634 | K.repcat.e.2008.body.300.Alu [symbol] | 10719 | AluJo.Alu.SINE.138391.[257] | 8.52E−02 |
| 635 | K.repcat.e&g.2008.body.300.L [symbol] | 4 | LTR9-int.ERV1.LTR.51.[921] | 8.88E−02 |
| 636 | K.repcat.e.2008.body.300.LTR [symbol] | 13 | LTR22.ERVK.LTR.103.[478] | 8.88E−02 |
| 637 | K.repcat.e&g.2008.body.300.L [symbol] | 29 | LTR10A.ERV1.LTR.301.[449] | 9.26E−02 |
| 638 | K.repcat.e&g.2008.body.300.L [symbol] | 4 | LTR8-int.ERV1.LTR.53.[752] | 9.26E−02 |
| 639 | K.repcat.e&g.2008.body.300.M [symbol] | 196 | MER81.AcHobo.DNA.3623.[94] | 9.26E−02 |
| 640 | K.repcat.e&g.2008.body.300.M [symbol] | 10 | MER83B- | 9.26E−02 |
| 641 | K.repcat.e&g.2008.body.300.P [symbol] | 8 | PRIMA4_LTR- | 9.26E−02 |
| 642 | K.repcat.e&g.2008.body.300.t [symbol] | 3 | tRNA-Arg- | 9.26E−02 |
| 643 | K.repcat.e.2008.body.300.tRN [symbol] | 1 | tRNA-Ser- | 9.26E−02 |
| 644 | K.repcat.e&g.2008.body.300.L [symbol] | 21 | L1M3a.L1.LINE.683.[590] | 9.64E−02 |
| 645 | K.repcat.e&g.2008.body.300.M [symbol] | 22 | MER102a.MER1_type.DNA.309.[2 [symbol] | 9.64E−02 |
| 646 | K.repcat.e&g.2008.body.300.M [symbol] | 581 | MER33.MER1_type.DNA.8040.[21 [symbol] | 9.64E−02 |
| 647 | K.repcat.e.2008.body.300.MER [symbol] | 17 | MER61F.ERV1.LTR.148.[438] | 9.64E−02 |
| 648 | K.repcat.e&g.2008.body.300.L [symbol] | 1 | LTR2-int.ERV1.LTR.5.[222] | 1.00E−01 |
| 649 | K.repcat.e&g.2008.body.300.M [symbol] | 91 | MER97c.MER1_type?.DNA.1428.[ [symbol] | 1.00E−01 |
| 650 | K.repcat.e&g.2008.body.300.L [symbol] | 26 | LTR3B_.ERVK.LTR.245.[336] | 1.05E−01 |
| 651 | K.repcat.e&g.2008.body.300.t [symbol] | 6 | tRNA-Leu- | 1.05E−01 |
| 652 | K.repcat.e&g.2008.body.300.A [symbol] | 10131 | AluJb.Alu.SINE.125688.[259] | 1.09E−01 |
| 653 | K.repcat.e&g.2008.body.300.L [symbol] | 43 | LTR26E.ERV1.LTR.307.[375] | 1.09E−01 |
| 654 | K.repcat.e&g.2008.body.300.R [symbol] | 96 | REP522.telo.Satellite.217.[6 [symbol] | 1.09E−01 |
| 655 | K.repcat.e.2008.body.300.LTR [symbol] | 29 | LTR28.ERV1.LTR.513.[593] | 1.09E−01 |
| 656 | K.repcat.e&g.2008.body.300.M [symbol] | 22 | MER73.ERVL.LTR.270.[408] | 1.13E−01 |
| 657 | K.repcat.e&g.2008.body.300.t [symbol] | 134 | telo | 1.13E−01 |
| 658 | K.repcat.e&g.2008.body.300.M [symbol] | 48 | MER61A- | 1.18E−01 |
| 659 | K.repcat.e&g.2008.body.300.c [symbol] | 315 | centr | 1.22E−01 |
| 660 | K.repcat.e&g.2008.body.300.C [symbol] | 163 | Charlie1b.MER1_type.DNA.1902 [symbol] | 1.22E−01 |
| 661 | K.repcat.e&g.2008.body.300.M [symbol] | 8 | MER39-int.ERV1.LTR.185.[359] | 1.22E−01 |
| 662 | K.repcat.e&g.2008.body.300.M [symbol] | 12 | MER61B- | 1.22E−01 |
| 663 | K.repcat.e&g.2008.body.300.S [symbol] | 921 | Satellite | 1.27E−01 |
| 664 | K.repcat.e&g.2008.body.300.L [symbol] | 4 | LTR19-int.ERV1.LTR.46.[890] | 1.32E−01 |
| 665 | K.repcat.e&g.2008.body.300.M [symbol] | 12 | MER99.DNA.DNA.277.[347] | 1.32E−01 |
| 666 | K.repcat.e&g.2008.body.300.t [symbol] | 1 | tRNA-Asn- | 1.32E−01 |
| 667 | K.repcat.e.2008.body.300.MER [symbol] | 58 | MER41D.ERV1.LTR.435.[299] | 1.32E−01 |
| 668 | K.repcat.e&g.2008.body.300.M [symbol] | 6 | MER41C- | 1.37E−01 |
| 669 | K.repcat.e&g.2008.body.300.t [symbol] | 7 | tRNA-Gly- | 1.37E−01 |
| 670 | K.repcat.e.2008.body.300.FLA [symbol] | 129 | FLAM.Alu.SINE.1401.[62] | 1.37E−01 |
| 671 | K.repcat.e.2008.body.300.GSA [symbol] | 50 | GSAT.centr.Satellite.65.[367 [symbol] | 1.37E−01 |
| 672 | K.repcat.e.2008.body.300.LTR [symbol] | 16 | LTR10E.ERV1.LTR.253.[458] | 1.37E−01 |
| 673 | K.repcat.e&g.2008.body.300.L [symbol] | 1 | LTR10B-int.ERV1.LTR.6.[901] | 1.42E−01 |
| 674 | K.repcat.e&g.2008.body.300.M [symbol] | 18 | MER97b.MER1_type?.DNA.194.[3 [symbol] | 1.42E−01 |
| 675 | K.repcat.e.2008.body.300.LTR [symbol] | 3363 | LTR12_.ERV1.LTR.664.[497] | 1.42E−01 |
| 676 | K.repcat.e.2008.body.300.MER [symbol] | 141 | MER21A.ERV1.LTR.1793.[398] | 1.42E−01 |
| 677 | K.repcat.e&g.2008.body.300.M [symbol] | 3 | MER83C-int.ERV1.LTR.39.[927] | 1.48E−01 |
| 678 | K.repcat.e&g.2008.body.300.t [symbol] | 13 | tRNA-Glu- | 1.48E−01 |
| 679 | K.repcat.e.2008.body.300.LTR [symbol] | 22 | LTR19C.ERV1.LTR.213.[410] | 1.48E−01 |
| 680 | K.repcat.e&g.2008.body.300.A [symbol] | 313 | AluJ/FLAM.Alu.SINE.3444.[78] | 1.53E−01 |
| 681 | K.repcat.e&g.2008.body.300.C [symbol] | 212 | Charlie4a.MER1_type.DNA.2329 [symbol] | 1.53E−01 |
| 682 | K.repcat.e&g.2008.body.300.M [symbol] | 141 | MER115.Tip100.DNA.2382.[290] | 1.53E−01 |
| 683 | K.repcat.e&g.2008.body.300.M [symbol] | 4408 | MIR3.MIR.SINE.70044.[121] | 1.53E−01 |
| 684 | K.repcat.e&g.2008.body.300.t [symbol] | 4 | tRNA-Ala- | 1.53E−01 |
| 685 | K.repcat.e.2008.body.300.LTR [symbol] | 37 | LTR24B.ERV1.LTR.457.[293] | 1.53E−01 |
| 686 | K.repcat.e&g.2008.body.300.H [symbol] | 51 | HY3.scRNA.scRNA.509.[87] | 1.59E−01 |
| 687 | K.repcat.e&g.2008.body.300.K [symbol] | 27 | Kanga1a.Tc2.DNA.310.[392] | 1.59E−01 |
| 688 | K.repcat.e&g.2008.body.300.M [symbol] | 16 | MER7D.MER2_type.DNA.397.[163 [symbol] | 1.59E−01 |
| 689 | K.repcat.e&g.2008.body.300.L [symbol] | 1 | LTR10A- | 1.65E−01 |
| 690 | K.repcat.e.2008.body.300.MER [symbol] | 60 | MER45.Tip100.DNA.678.[86] | 1.65E−01 |
| 691 | K.repcat.e&g.2008.body.300.H [symbol] | 87 | HERVIP10FH.ERV1.LTR.594.[104 [symbol] | 1.77E−01 |
| 692 | K.repcat.e&g.2008.body.300.L [symbol] | 34 | LTR5A.ERVK.LTR.250.[947] | 1.77E−01 |
| 693 | K.repcat.e&g.2008.body.300.t [symbol] | 2 | tRNA-Leu- | 1.77E−01 |
| 694 | K.repcat.e.2008.body.300.MER [symbol] | 70 | MER34C_.ERV1.LTR.1038.[282] | 1.77E−01 |
| 695 | K.repcat.e.2008.body.300.MER [symbol] | 80 | MER93B.ERV1.LTR.989.[269] | 1.77E−01 |
| 696 | K.repcat.e&g.2008.body.300.A [symbol] | 283 | AluSp/q.Alu.SINE.2568.[100] | 1.83E−01 |
| 697 | K.repcat.e&g.2008.body.300.A [symbol] | 15 | AluYb8.Alu.SINE.68.[161] | 1.83E−01 |
| 698 | K.repcat.e&g.2008.body.300.C [symbol] | 5 | Charlie11.MER1_type.DNA.87.[ [symbol] | 1.83E−01 |
| 699 | K.repcat.e&g.2008.body.300.P [symbol] | 13 | PRIMA41- | 1.83E−01 |
| 700 | K.repcat.e.2008.body.300.LTR [symbol] | 51 | LTR52.ERVL.LTR.1035.[277] | 1.83E−01 |
| 701 | K.repcat.e.2008.body.300.MER [symbol] | 94 | MER4E1.ERV1.LTR.1057.[396] | 1.83E−01 |
| 702 | K.repcat.e&g.2008.body.300.M [symbol] | 51 | MLT1G3.MaLR.LTR.596.[291] | 1.90E−01 |
| 703 | K.repcat.e&g.2008.body.300.L [symbol] | 1 | LTR29-int.ERV1.LTR.55.[532] | 1.96E−01 |
| 704 | K.repcat.e&g.2008.body.300.M [symbol] | 2 | MER110A- | 1.96E−01 |
| 705 | K.repcat.e&g.2008.body.300.M [symbol] | 215 | MER96B.MER1_type?.DNA.2121.[ [symbol] | 1.96E−01 |
| 706 | K.repcat.e.2008.body.300.LTR [symbol] | 12 | LTR3.ERVK.LTR.83.[383] | 1.96E−01 |
| 707 | K.repcat.e&g.2008.body.300.U [symbol] | 53 | U2.snRNA.snRNA.1053.[44] | 2.03E−01 |
| 708 | K.repcat.e&g.2008.body.300.L [symbol] | 91 | LTR2C.ERV1.LTR.237.[343] | 2.10E−01 |
| 709 | K.repcat.e&g.2008.body.300.U [symbol] | 1 | U8.snRNA.snRNA.30.[110] | 2.10E−01 |
| 710 | K.repcat.e&g.2008.body.300.M [symbol] | 62 | MER45C.Tip100.DNA.1080.[297] | 2.17E−01 |
| 711 | K.repcat.e&g.2008.body.300.t [symbol] | 7 | tRNA-Phe- | 2.17E−01 |
| 712 | K.repcat.e&g.2008.body.300.L [symbol] | 38 | LTR62.ERVL.LTR.312.[425] | 2.24E−01 |
| 713 | K.repcat.e&g.2008.body.300.P [symbol] | 10 | PRIMAX- | 2.24E−01 |
| 714 | K.repcat.e&g.2008.body.300.H [symbol] | 3 | HERVK11D.ERVK.LTR.57.[1690] | 2.32E−01 |
| 715 | K.repcat.e&g.2008.body.300.M [symbol] | 9 | MER66B- | 2.32E−01 |
| 716 | K.repcat.e&g.2008.body.300.M [symbol] | 7 | MER70-int.ERVL.LTR.124.[401] | 2.32E−01 |
| 717 | K.repcat.e.2008.body.300.LTR [symbol] | 71 | LTR23.ERV1.LTR.840.[205] | 2.32E−01 |
| 718 | K.repcat.e&g.2008.body.300.C [symbol] | 29 | Charlie2b.MER1_type.DNA.502. [symbol] | 2.39E−01 |
| 719 | K.repcat.e.2008.body.300.tRN [symbol] | 6 | tRNA-Ala- | 2.39E−01 |
| 720 | K.repcat.e&g.2008.body.300.M [symbol] | 192 | MER117.MER1_type.DNA.4409.[1 [symbol] | 2.47E−01 |
| 721 | K.repcat.e&g.2008.body.300.M [symbol] | 16 | MER97a.MER1_type?.DNA.237.[3 [symbol] | 2.47E−01 |
| 722 | K.repcat.e.2008.body.300.tRN [symbol] | 2 | tRNA-Ser- | 2.47E−01 |
| 723 | K.repcat.e&g.2008.body.300.L [symbol] | 5 | LTR38-int.ERV1.LTR.126.[481] | 2.55E−01 |
| 724 | K.repcat.e&g.2008.body.300.L [symbol] | 3 | LTR69.ERVL.LTR.140.[376] | 2.55E−01 |
| 725 | K.repcat.e&g.2008.body.300.M [symbol] | 101 | MER104.Tc2.DNA.2013.[149] | 2.55E−01 |
| 726 | K.repcat.e&g.2008.body.300.t [symbol] | 3 | tRNA-Pro- | 2.55E−01 |
| 727 | K.repcat.e&g.2008.body.300.B [symbol] | 24 | BC200.scRNA.scRNA.253.[134] | 2.63E−01 |
| 728 | K.repcat.e&g.2008.body.300.C [symbol] | 4 | CER.Satellite.Satellite.35.[ [symbol] | 2.63E−01 |
| 729 | K.repcat.e&g.2008.body.300.C [symbol] | 17 | Charlie12.MER1_type.DNA.164. [symbol] | 2.63E−01 |
| 730 | K.repcat.e&g.2008.body.300.T [symbol] | 50 | Tigger8.MER2_type.DNA.837.[2 [symbol] | 2.63E−01 |
| 731 | K.repcat.e&g.2008.body.300.L [symbol] | 27 | LTR42.ERVL.LTR.287.[361] | 2.72E−01 |
| 732 | K.repcat.e&g.2008.body.300.L [symbol] | 12 | LTR10B1- | 2.80E−01 |
| 733 | K.repcat.e&g.2008.body.300.L [symbol] | 111 | LTR5B.ERVK.LTR.420.[781] | 2.80E−01 |
| 734 | K.repcat.e&g.2008.body.300.M [symbol] | 70 | MER69A.Tip100.DNA.1046.[140] | 2.89E−01 |
| 735 | K.repcat.e.2008.body.300.HER [symbol] | 116 | HERVK.SVA, ERVK.Other, LTR.361 [symbol] | 2.89E−01 |
| 736 | K.repcat.e&g.2008.body.300.7 [symbol] | 99 | 7SLRNA.srpRNA.srpRNA.937.[24 [symbol] | 2.98E−01 |
| 737 | K.repcat.e&g.2008.body.300.F [symbol] | 2632 | FLAM_C.Alu.SINE.34861.[125] | 2.98E−01 |
| 738 | K.repcat.e&g.2008.body.300.L [symbol] | 39 | LTR26B.ERV1.LTR.436.[324] | 2.98E−01 |
| 739 | K.repcat.e&g.2008.body.300.s [symbol] | 99 | srpRNA | 2.98E−01 |
| 740 | K.repcat.e&g.2008.body.300.t [symbol] | 1 | tRNA-Ser- | 2.98E−01 |
| 741 | K.repcat.e.2008.body.300.MER [symbol] | 95 | MER51A.ERV1.LTR.953.[414] | 2.98E−01 |
| 742 | K.repcat.e.2008.body.300.MER [symbol] | 67 | MER96.MER1_type?.DNA.1232.[1 [symbol] | 2.98E−01 |
| 743 | K.repcat.e&g.2008.body.300.F [symbol] | 1265 | FLAM_A.Alu.SINE.17781.[117] | 3.07E−01 |
| 744 | K.repcat.e&g.2008.body.300.M [symbol] | 33 | MER21-int.ERV1.LTR.385.[455] | 3.07E−01 |
| 745 | K.repcat.e&g.2008.body.300.M [symbol] | 44 | MER91C.Tip100.DNA.1249.[92] | 3.07E−01 |
| 746 | K.repcat.e.2008.body.300.LTR [symbol] | 2691 | LTR12C.ERV1.LTR.2616.[1340] | 3.07E−01 |
| 747 | K.repcat.e&g.2008.body.300.L [symbol] | 85 | LTR12E.ERV1.LTR.127.[781] | 3.16E−01 |
| 748 | K.repcat.e&g.2008.body.300.M [symbol] | 70 | MLT2E.ERVL.LTR.1362.[250] | 3.16E−01 |
| 749 | K.repcat.e.2008.body.300.LTR [symbol] | 30 | LTR75.ERVL.LTR.404.[321] | 3.16E−01 |
| 750 | K.repcat.e.2008.body.300.MER [symbol] | 97 | MER31B.ERV1.LTR.1459.[309] | 3.16E−01 |
| 751 | K.repcat.e&g.2008.body.300.E [symbol] | 4 | ERVL-B2.ERVL.LTR.153.[1038] | 3.25E−01 |
| 752 | K.repcat.e&g.2008.body.300.M [symbol] | 363 | MER113.MER1_type.DNA.4474.[1 [symbol] | 3.25E−01 |
| 753 | K.repcat.e&g.2008.body.300.t [symbol] | 3 | tRNA-Pro- | 3.25E−01 |
| 754 | K.repcat.e.2008.body.300.LTR [symbol] | 10 | LTR45.ERV1.LTR.179.[297] | 3.25E−01 |
| 755 | K.repcat.e&g.2008.body.300.L [symbol] | 5 | LTR58.ERV1.LTR.83.[344] | 3.35E−01 |
| 756 | K.repcat.e&g.2008.body.300.M [symbol] | 72 | MER92B.ERV1.LTR.870.[297] | 3.35E−01 |
| 757 | K.repcat.e&g.2008.body.300.U [symbol] | 26 | U13_.snRNA.snRNA.304.[98] | 3.35E−01 |
| 758 | K.repcat.e&g.2008.body.300.U [symbol] | 7 | U5.snRNA.snRNA.205.[55] | 3.35E−01 |
| 759 | K.repcat.e&g.2008.body.300.t [symbol] | 3 | tRNA-Arg- | 3.44E−01 |
| 760 | K.repcat.e.2008.body.300.Tig [symbol] | 19 | Tigger6.MER2_type.DNA.253.[2 [symbol] | 3.44E−01 |
| 761 | K.repcat.e&g.2008.body.300.L [symbol] | 10 | LTR21B.ERV1.LTR.58.[342] | 3.64E−01 |
| 762 | K.repcat.e&g.2008.body.300.M [symbol] | 6 | MLT1F2- | 3.64E−01 |
| 763 | K.repcat.e&g.2008.body.300.A [symbol] | 118 | AluJo/FRAM.Alu.SINE.1339.[14 [symbol] | 3.75E−01 |
| 764 | K.repcat.e&g.2008.body.300.L [symbol] | 2 | LTR10E-int.ERV1.LTR.8.[311] | 3.75E−01 |
| 765 | K.repcat.e&g.2008.body.300.Z [symbol] | 45 | Zaphod2.Tip100.DNA.715.[196] | 3.75E−01 |
| 766 | K.repcat.e.2008.body.300.Cha [symbol] | 91 | Charlie4.MER1_type.DNA.1363. [symbol] | 3.75E−01 |
| 767 | K.repcat.e.2008.body.300.MER [symbol] | 39 | MER83.ERV1.LTR.435.[329] | 3.75E−01 |
| 768 | K.repcat.e.2008.body.300.LTR [symbol] | 26 | LTR19A.ERV1.LTR.417.[359] | 3.85E−01 |
| 769 | K.repcat.e&g.2008.body.300.A [symbol] | 21 | AluJ/monomer.Alu.SINE.329.[5 [symbol] | 3.96E−01 |
| 770 | K.repcat.e&g.2008.body.300.L [symbol] | 35 | LTR18B.ERVL.LTR.382.[423] | 3.96E−01 |
| 771 | K.repcat.e&g.2008.body.300.L [symbol] | 23 | LTR52-int.ERVL.LTR.338.[508] | 3.96E−01 |
| 772 | K.repcat.e&g.2008.body.300.M [symbol] | 3 | MER52-int.ERV1.LTR.136.[864] | 3.96E−01 |
| 773 | K.repcat.e&g.2008.body.300.M [symbol] | 332 | MER53.DNA.DNA.5686.[128] | 3.96E−01 |
| 774 | K.repcat.e&g.2008.body.300.L [symbol] | 822 | L1MB5.L1.LINE.8813.[405] | 4.06E−01 |
| 775 | K.repcat.e&g.2008.body.300.L [symbol] | 7 | LTR24B- | 4.06E−01 |
| 776 | K.repcat.e&g.2008.body.300.L [symbol] | 23 | LTR44.ERV1.LTR.201.[309] | 4.06E−01 |
| 777 | K.repcat.e&g.2008.body.300.H [symbol] | 42 | HERVL18.ERVL.LTR.531.[760] | 4.17E−01 |
| 778 | K.repcat.e&g.2008.body.300.L [symbol] | 34 | LTR35.ERV1.LTR.306.[301] | 4.17E−01 |
| 779 | K.repcat.e&g.2008.body.300.L [symbol] | 34 | LTR65.ERV1.LTR.452.[268] | 4.17E−01 |
| 780 | K.repcat.e&g.2008.body.300.M [symbol] | 7 | MSTB1-int.MaLR.LTR.179.[670] | 4.17E−01 |
| 781 | K.repcat.e&g.2008.body.300.t [symbol] | 4 | tRNA-Tyr- | 4.17E−01 |
| 782 | K.repcat.e.2008.body.300.MER [symbol] | 79 | MER89.ERV1.LTR.1339.[310] | 4.17E−01 |
| 783 | K.repcat.e&g.2008.body.300.L [symbol] | 4 | LTR19B-int.ERV1.LTR.43.[730] | 4.28E−01 |
| 784 | K.repcat.e&g.2008.body.300.M [symbol] | 271 | MER102b.MER1_type.DNA.3930.[ [symbol] | 4.28E−01 |
| 785 | K.repcat.e.2008.body.300.LTR [symbol] | 50 | LTR10C.ERV1.LTR.507.[462] | 4.28E−01 |
| 786 | K.repcat.e&g.2008.body.300.L [symbol] | 383 | L3b.CR1.LINE.7618.[138] | 4.40E−01 |
| 787 | K.repcat.e&g.2008.body.300.L [symbol] | 98 | LTR13A.ERVK.LTR.182.[795] | 4.40E−01 |
| 788 | K.repcat.e&g.2008.body.300.L [symbol] | 123 | LTR13_.ERVK.LTR.36.[891] | 4.40E−01 |
| 789 | K.repcat.e&g.2008.body.300.L [symbol] | 5 | LTR19C- | 4.40E−01 |
| 790 | K.repcat.e&g.2008.body.300.M [symbol] | 2084 | MIRm.MIR.SINE.32156.[90] | 4.51E−01 |
| 791 | K.repcat.e&g.2008.body.300.P [symbol] | 11 | PABL_B- | 4.51E−01 |
| 792 | K.repcat.e.2008.body.300.For [symbol] | 26 | FordPrefect.Tip100.DNA.410.[ [symbol] | 4.51E−01 |
| 793 | K.repcat.e.2008.body.300.LTR [symbol] | 7 | LTR10B.ERV1.LTR.70.[433] | 4.51E−01 |
| 794 | K.repcat.e.2008.body.300.MER [symbol] | 27 | MER34D.ERV1.LTR.339.[322] | 4.51E−01 |
| 795 | K.repcat.e.2008.body.300.MER [symbol] | 26 | MER51E.ERV1.LTR.240.[301] | 4.51E−01 |
| 796 | K.repcat.e&g.2008.body.300.A [symbol] | 270 | AluJo/FLAM.Alu.SINE.2189.[11 [symbol] | 4.63E−01 |
| 797 | K.repcat.e&g.2008.body.300.M [symbol] | 2 | MER67A-int.ERV1.LTR.18.[278] | 4.63E−01 |
| 798 | K.repcat.e&g.2008.body.300.t [symbol] | 9 | tRNA-Glu- | 4.63E−01 |
| 799 | K.repcat.e.2008.body.300.LTR [symbol] | 205 | LTR13.ERVK.LTR.458.[836] | 4.63E−01 |
| 800 | K.repcat.e.2008.body.300.MER [symbol] | 53 | MER93a.ERV1.LTR.311.[247] | 4.63E−01 |
| 801 | K.repcat.e&g.2008.body.300.M [symbol] | 11 | MER88.ERVL.LTR.110.[327] | 4.74E−01 |
| 802 | K.repcat.e.2008.body.300.MER [symbol] | 32 | MER101.ERV1.LTR.379.[292] | 4.74E−01 |
| 803 | K.repcat.e.2008.body.300.MER [symbol] | 59 | MER90.ERV1.LTR.678.[317] | 4.74E−01 |
| 804 | K.repcat.e&g.2008.body.300.H [symbol] | 16 | HERVK13.ERVK.LTR.64.[1278] | 4.86E−01 |
| 805 | K.repcat.e.2008.body.300.MER [symbol] | 56 | MER67B.ERV1.LTR.438.[353] | 4.98E−01 |
| 806 | K.repcat.e&g.2008.body.300.H [symbol] | 192 | HERVE.ERV1.LTR.346.[2130] | 5.11E−01 |
| 807 | K.repcat.e&g.2008.body.300.M [symbol] | 31 | MER45R.Tip100.DNA.260.[525] | 5.11E−01 |
| 808 | K.repcat.e.2008.body.300.LTR [symbol] | 16 | LTR25.ERV1.LTR.261.[466] | 5.11E−01 |
| 809 | K.repcat.e&g.2008.body.300.T [symbol] | 27 | Tigger5c.MER2_type.DNA.939.[ [symbol] | 5.23E−01 |
| 810 | K.repcat.e&g.2008.body.300.M [symbol] | 67 | MER106B.MER1_type.DNA.839.[1 [symbol] | 5.35E−01 |
| 811 | K.repcat.e&g.2008.body.300.M [symbol] | 1 | MER34D-int.ERV1.LTR.24.[999] | 5.35E−01 |
| 812 | K.repcat.e&g.2008.body.300.M [symbol] | 45 | MER106A.MER1_type.DNA.689.[1 [symbol] | 5.48E−01 |
| 813 | K.repcat.e&g.2008.body.300.G [symbol] | 73 | GSATII.centr.Satellite.157.[ [symbol] | 5.61E−01 |
| 814 | K.repcat.e&g.2008.body.300.H [symbol] | 27 | HERV15.ERV1.LTR.116.[1879] | 5.61E−01 |
| 815 | K.repcat.e&g.2008.body.300.M [symbol] | 93 | MER48.ERV1.LTR.192.[361] | 5.61E−01 |
| 816 | K.repcat.e.2008.body.300.HER [symbol] | 48 | HERV3.ERV1.LTR.256.[1560] | 5.61E−01 |
| 817 | K.repcat.e&g.2008.body.300.L [symbol] | 7 | LTR46-int.ERV1.LTR.67.[1415] | 5.74E−01 |
| 818 | K.repcat.e&g.2008.body.300.M [symbol] | 216 | MER91A.Tip100.DNA.3430.[124] | 5.74E−01 |
| 819 | K.repcat.e&g.2008.body.300.U [symbol] | 24 | U7.snRNA.snRNA.246.[54] | 5.87E−01 |
| 820 | K.repcat.e.2008.body.300.Alu [symbol] | 30 | AluYg.Alu.SINE.111.[105] | 5.87E−01 |
| 821 | K.repcat.e.2008.body.300.LTR [symbol] | 40 | LTR34.ERV1.LTR.361.[370] | 5.87E−01 |
| 822 | K.repcat.e&g.2008.body.300.H [symbol] | 10 | HY4.scRNA.scRNA.157.[79] | 6.00E−01 |
| 823 | K.repcat.e&g.2008.body.300.t [symbol] | 2 | tRNA-Arg- | 6.00E−01 |
| 824 | K.repcat.e&g.2008.body.300.H [symbol] | 60 | HERVFH21.ERV1.LTR.183.[962] | 6.14E−01 |
| 825 | K.repcat.e&g.2008.body.300.t [symbol] | 12 | tRNA-Gly- | 6.14E−01 |
| 826 | K.repcat.e&g.2008.body.300.F [symbol] | 19 | FordPrefect_a.Tip100.DNA.398 [symbol] | 6.27E−01 |
| 827 | K.repcat.e&g.2008.body.300.S [symbol] | 236 | SATR1.Satellite.Satellite.42 [symbol] | 6.27E−01 |
| 828 | K.repcat.e.2008.body.300.LTR [symbol] | 28 | LTR24.ERV1.LTR.308.[302] | 6.27E−01 |
| 829 | K.repcat.e&g.2008.body.300.L [symbol] | 1 | LTR24C-int.ERV1.LTR.46.[510] | 6.41E−01 |
| 830 | K.repcat.e&g.2008.body.300.L [symbol] | 16 | LTR61.ERV1.LTR.149.[398] | 6.41E−01 |
| 831 | K.repcat.e&g.2008.body.300.M [symbol] | 243 | MER45A.Tip100.DNA.3214.[141] | 6.41E−01 |
| 832 | K.repcat.e&g.2008.body.300.M [symbol] | 28 | MER51C.ERV1.LTR.180.[396] | 6.41E−01 |
| 833 | K.repcat.e.2008.body.300.LTR [symbol] | 19 | LTR14.ERVK.LTR.83.[463] | 6.41E−01 |
| 834 | K.repcat.e&g.2008.body.300.O [symbol] | 1653 | Other | 6.68E−01 |
| 835 | K.repcat.e&g.2008.body.300.S [symbol] | 1653 | SVA | 6.68E−01 |
| 836 | K.repcat.e.2008.body.300.LTR [symbol] | 5 | LTR5.ERVK.LTR.23.[192] | 6.68E−01 |
| 837 | K.repcat.e&g.2008.body.300.M [symbol] | 13 | MER51D.ERV1.LTR.148.[239] | 6.82E−01 |
| 838 | K.repcat.e&g.2008.body.300.M [symbol] | 35264 | MIR.MIR.SINE.203898.[147] | 6.82E−01 |
| 839 | K.repcat.e&g.2008.body.300.t [symbol] | 2 | tRNA-Met-i.tRNA.tRNA.38.[60] | 6.82E−01 |
| 840 | K.repcat.e&g.2008.body.300.L [symbol] | 4 | LTR60-int.ERV1.LTR.19.[368] | 6.96E−01 |
| 841 | K.repcat.e.2008.body.300.U13 [symbol] | 9 | U13.snRNA.snRNA.178.[98] | 6.96E−01 |
| 842 | K.repcat.e&g.2008.body.300.L [symbol] | 2 | LTR38B-int.ERV1.LTR.18.[385] | 7.10E−01 |
| 843 | K.repcat.e&g.2008.body.300.M [symbol] | 66 | MER91B.Tip100.DNA.1429.[105] | 7.10E−01 |
| 844 | K.repcat.e.2008.body.300.LTR [symbol] | 71 | LTR6A.ERV1.LTR.260.[460] | 7.10E−01 |
| 845 | K.repcat.e&g.2008.body.300.F [symbol] | 48 | FRAM/FAM.Alu.SINE.530.[68] | 7.25E−01 |
| 846 | K.repcat.e&g.2008.body.300.L [symbol] | 1 | LTR30-int.ERV1.LTR.7.[1505] | 7.25E−01 |
| 847 | K.repcat.e&g.2008.body.300.L [symbol] | 44 | LTR6B.ERV1.LTR.149.[506] | 7.25E−01 |
| 848 | K.repcat.e&g.2008.body.300.M [symbol] | 11 | MER51A- | 7.25E−01 |
| 849 | K.repcat.e&g.2008.body.300.M [symbol] | 28 | MER63B.AcHobo.DNA.544.[368] | 7.25E−01 |
| 850 | K.repcat.e&g.2008.body.300.H [symbol] | 20 | HERVFH19.ERV1.LTR.135.[947] | 7.39E−01 |
| 851 | K.repcat.e&g.2008.body.300.H [symbol] | 4 | HERVL-B3.ERVL.LTR.93.[1286] | 7.39E−01 |
| 852 | K.repcat.e&g.2008.body.300.L [symbol] | 24844 | L2.L2.LINE.403973.[230] | 7.39E−01 |
| 853 | K.repcat.e&g.2008.body.300.M [symbol] | 17373 | MIRb.MIR.SINE.278083.[144] | 7.39E−01 |
| 854 | K.repcat.e&g.2008.body.300.t [symbol] | 13 | tRNA-Cys- | 7.39E−01 |
| 855 | K.repcat.e.2008.body.300.MER [symbol] | 100 | MER31A.ERV1.LTR.1316.[321] | 7.39E−01 |
| 856 | K.repcat.e&g.2008.body.300.L [symbol] | 19 | LTR21A.ERV1.LTR.89.[418] | 7.53E−01 |
| 857 | K.repcat.e.2008.body.300.LTR [symbol] | 13 | LTR70.ERV1.LTR.128.[673] | 7.53E−01 |
| 858 | K.repcat.e&g.2008.body.300.D [symbol] | 18 | D20S16.Satellite.Satellite.2 [symbol] | 7.68E−01 |
| 859 | K.repcat.e&g.2008.body.300.L [symbol] | 31 | LTR10D.ERV1.LTR.189.[431] | 7.68E−01 |
| 860 | K.repcat.e&g.2008.body.300.L [symbol] | 3 | LTR10G- | 7.68E−01 |
| 861 | K.repcat.e&g.2008.body.300.L [symbol] | 27 | LTR47B.ERVL.LTR.380.[336] | 7.68E−01 |
| 862 | K.repcat.e&g.2008.body.300.M [symbol] | 10 | MER4A-int.ERV1.LTR.120.[826] | 7.68E−01 |
| 863 | K.repcat.e&g.2008.body.300.R [symbol] | 10 | Ricksha_b.MuDR.DNA.115.[592] | 7.97E−01 |
| 864 | K.repcat.e&g.2008.body.300.M [symbol] | 1 | MER65D-int.ERV1.LTR.77.[587] | 8.12E−01 |
| 865 | K.repcat.e&g.2008.body.300.t [symbol] | 11 | tRNA-Lys- | 8.12E−01 |
| 866 | K.repcat.e&g.2008.body.300.H [symbol] | 58 | HERVH48.ERV1.LTR.115.[1792] | 8.27E−01 |
| 867 | K.repcat.e&g.2008.body.300.H [symbol] | 83 | HERVS71.ERV1.LTR.149.[1952] | 8.27E−01 |
| 868 | K.repcat.e&g.2008.body.300.M [symbol] | 5 | MER41D-int.ERV1.LTR.68.[425] | 8.27E−01 |
| 869 | K.repcat.e&g.2008.body.300.M [symbol] | 116 | MER74A.ERVL.LTR.1526.[372] | 8.27E−01 |
| 870 | K.repcat.e.2008.body.300.Alu [symbol] | 5 | AluYc.Alu.SINE.50.[73] | 8.27E−01 |
| 871 | K.repcat.e&g.2008.body.300.M [symbol] | 7 | MER66-int.ERV1.LTR.249.[529] | 8.56E−01 |
| 872 | K.repcat.e.2008.body.300.MER [symbol] | 1109 | MER20.MER1_type.DNA.16178.[1 [symbol] | 8.71E−01 |
| 873 | K.repcat.e&g.2008.body.300.L [symbol] | 2 | LTR1B-int.ERV1.LTR.11.[2238] | 8.86E−01 |
| 874 | K.repcat.e&g.2008.body.300.s [symbol] | 296 | snRNA | 8.86E−01 |
| 875 | K.repcat.e&g.2008.body.300.T [symbol] | 4 | Tigger6b.MER2_type.DNA.96.[4 [symbol] | 8.86E−01 |
| 876 | K.repcat.e&g.2008.body.300.M [symbol] | 21 | MER58D.MER1_type.DNA.282.[25 [symbol] | 9.01E−01 |
| 877 | K.repcat.e&g.2008.body.300.T [symbol] | 40 | TAR1.telo.Satellite.123.[482 [symbol] | 9.17E−01 |
| 878 | K.repcat.e&g.2008.body.300.t [symbol] | 3 | tRNA-Gln- | 9.17E−01 |
| 879 | K.repcat.e&g.2008.body.300.5 [symbol] | 107 | 5S.rRNA.rRNA.1258.[70] | 9.32E−01 |
| 880 | K.repcat.e&g.2008.body.300.L [symbol] | 19 | LTR31.ERV1.LTR.249.[407] | 9.32E−01 |
| 881 | K.repcat.e&g.2008.body.300.L [symbol] | 2 | LTR6A-int.ERV1.LTR.15.[542] | 9.32E−01 |
| 882 | K.repcat.e&g.2008.body.300.L [symbol] | 28 | LTR71B.ERV1.LTR.209.[426] | 9.32E−01 |
| 883 | K.repcat.e&g.2008.body.300.r [symbol] | 107 | rRNA | 9.32E−01 |
| 884 | K.repcat.e.2008.body.300.Alu [symbol] | 9 | AluYh.Alu.SINE.23.[120] | 9.32E−01 |
| 885 | K.repcat.e.2008.body.300.MER [symbol] | 35 | MER52C.ERV1.LTR.366.[827] | 9.32E−01 |
| 886 | K.repcat.e&g.2008.body.300.M [symbol] | 54 | MER121.Unknown.Unknown.919.[ [symbol] | 9.47E−01 |
| 887 | K.repcat.e&g.2008.body.300.U [symbol] | 10 | U4.snRNA.snRNA.161.[92] | 9.47E−01 |
| 888 | K.repcat.e.2008.body.300.Unk [symbol] | 54 | Unknown | 9.47E−01 |
| 889 | K.repcat.e&g.2008.body.300.L [symbol] | 7 | LOR1a-int.ERV1.LTR.215.[673] | 9.62E−01 |
| 890 | K.repcat.e&g.2008.body.300.t [symbol] | 2 | tRNA-Val- | 9.77E−01 |
| 891 | K.repcat.e.2008.body.300.LTR [symbol] | 27 | LTR10B1.ERV1.LTR.242.[314] | 9.77E−01 |
| 892 | K.repcat.e&g.2008.body.300.C [symbol] | 569 | Charlie8.MER1_type.DNA.7585. [symbol] | 9.92E−01 |
| 893 | K.repcat.e&g.2008.body.300.G [symbol] | 25 | GSATX.centr.Satellite.58.[17 [symbol] | 9.92E−01 |
| 894 | K.repcat.e&g.2008.body.300.L [symbol] | 4 | LTR7A.ERV1.LTR.16.[122] | 9.92E−01 |
| 895 | K.repcat.e&g.2008.body.300.M [symbol] | 91 | MER90a.ERV1.LTR.1165.[326] | 9.92E−01 |
| 896 | K.repcat.e&g.2008.body.300.t [symbol] | 1 | tRNA-SeC(e)- | 9.92E−01 |
| 897 | K.repcat.e.2008.body.300.LTR [symbol] | 15 | LTR72.ERV1.LTR.149.[355] | 9.92E−01 |
| 898 | K.repcat.e.2008.body.300.tRN [symbol] | 1 | tRNA-Thr- | 9.92E−01 |

[symbol]

indicates data missing or illegible when filed

Next, statistical analysis was performed using random forest binary decision trees. Table 6 shows the importance of top 45 from 139,379 variables generated using Random Forest algorithm. The categories include gene probes (gene), non-genic and non-repetitive probes (nonrep), repetitive element. The random forest classifier based on the repetitive element categories alone worked well (89% accuracy). Both algorithms agree on several categories of repetitive elements being the most informative, i.e. both algorithms report them in the top 20, for example: MER67D, HUERS-P3B, MER6, MER66C, ERVL, MLT1G1, MLT2D, MER50B, THE1B (Table 5 and Table 7). In both analyses, the categories based on the primer design discussed in recent literature ranked much lower i.e. ˜200 (Table 5, Wilcoxon) or ˜350 (Table 7, Random Forest) than the categories defined based on repetitive elements.

TABLE 6
Importance of top 45 from 139,379 variables generated using Random
Forest algorithm. The categories include gene probes (gene), non-genic and non-repetitive
probes (nonrep), repetitive element (description includes name, class and family of a
repetitive element according to RepBase, count of a repeat in the genome and [average
length]). The ranking is based on the average decrease in classification accuracy if the
variables are randomly permuted one at a time. The meanMargin and meanTumor columns
show the methylation level in category among Non-Tumor Adjacent and Tumor experiments
respectively. Columns 1 (Normal), 2 (Non-Tumor Adjacent), and 3 (Tumor) indicate the
decrease in the prediction accuracy per category in a given subset of experiments during the
cross validation. MeanDecreaseAccuracy is an average decrease of overall accuracy of
classification. MeanDecreaseGini indicates average decrease in Gini statistic
(summarized in the ROC in FIG. 4).
Importance of top 45 variables (total 139379 Variables)

| | Num | Mean | Mean | | | | Mean Decrease |
|---|---|---|---|---|---|---|---|
| | Probe | Margin | Tumor | 1 | 2 | 3 | Accuracy |
| nonrep.122208 | 1 | −0.713 | −4.307 | 6.616 | 5.741 | 4.621 | 3.387 |
| nonrep.122209 | 1 | −0.389 | −3.732 | 5.687 | 4.706 | 3.919 | 2.843 |
| MER6.MER2_type.DNA.926.(416) | 1995 | 0.041 | 0.13 | 5.432 | 4.354 | 3.857 | 2.788 |
| HUERS-P3b.ERV1.LTR.378.(917) | 28 | 0.292 | 0.517 | 5.96 | 4.247 | 3.761 | 2.743 |
| MER66C.ERV1.LTR.661.(279) | 54 | 0.063 | 0.217 | 5.685 | 4.446 | 3.622 | 2.673 |
| MER67D.ERV1.LTR.1130.(294) | 49 | 0.21 | 0.411 | 5.575 | 3.094 | 3.687 | 2.637 |
| ERVL.ERVL.LTR.826.(422) | 6129 | 0.119 | 0.212 | 5.277 | 3.808 | 3.389 | 2.511 |
| MER4D.ERV1.LTR.1275.(421) | 247 | 0.081 | 0.171 | 5.476 | 2.978 | 3.199 | 2.336 |
| MLT1A1.MaLR.LTR.6611.(294) | 321 | 0.094 | 0.209 | 5.221 | 3.786 | 3.116 | 2.334 |
| MLT1G1.MaLR.LTR.4437.(320) | 247 | 0.069 | 0.182 | 4.424. | 3.43 | 3.08 | 2.298 |
| MLT2B3.ERVL.LTR.2966.(406) | 132 | 0.244 | 0.424 | 5.356 | 1.891 | 3.161 | 2.295 |
| nonrep.303433 | 1 | 0.247 | −2.016 | 1.695 | 3.929 | 3.109 | 2.29 |
| MLT2D.ERVL.LTR.4035.(266) | 174 | 0.121 | 0.218 | 4.386 | 2.862 | 3.224 | 2.273 |
| body.LTR | 38991 | 0.178 | 0.272 | 5.166 | 2.442 | 3.024 | 2.263 |
| body.MaLR | 15574 | 0.126 | 0.215 | 4.747 | 2.685 | 3.128 | 2.259 |
| gene.122206 | 1 | 0.382 | −2.091 | 4.592 | 3.842 | 2.985 | 2.251 |
| gene.122207 | 1 | −1.391 | −3.43 | 4.732 | 3.499 | 3.059 | 2.225 |
| nonrep.122204 | 1 | −1.093 | −4.179 | 3.721 | 2.97 | 2.958 | 2.214 |
| non rep.70135 | 1 | −1.109 | −1.717 | 4.871 | 1.954 | 2.992 | 2.212 |
| nonrep.147762 | 1 | −0.068 | −1.766 | 3.67 | 3.441 | 2.836 | 2.128 |
| THE1B.MaLR.LTR.22106.(329) | 719 | 0.316 | 0.461 | 5.404 | 2.201 | 2.843 | 2.122 |
| gene.54663 | 1 | 0.336 | −2.149 | 2.139 | 3.89 | 2.681 | 2.095 |
| gene.298679 | 1 | −1.309 | −2.11 | 4.702 | 3.006 | 2.702 | 2.079 |
| gene.232740 | 1 | −0.143 | −1.737 | 3.671 | 2.961 | 2.848 | 2.071 |
| nonrep.154468 | 1 | 1.317 | −1.228 | −0.708 | 3.816 | 2.708 | 2.056 |
| gene.70781 | 1 | 0.211 | −1.791 | 2.582 | 3.684 | 2.715 | 2.03 |
| MLT1A.MaLR.LTR.8650.(280) | 1783 | 0.107 | 0.215 | 4.085 | 2.908 | 2.76 | 2.014 |
| MLT2B2.ERVL.LTR.2019.(335) | 91 | −0.07 | 0.1 | −0.36 | 3.804 | 2.577 | 1.993 |
| MER50B.ERV1.LTR.681.(468) | 102 | 0.073 | 0.262 | 3.091 | 3.004 | 2.722 | 1.992 |
| gene.232742 | 1 | −0.485 | −2.134 | 3.006 | 3.569 | 2.631 | 1.99 |
| gene.91801 | 1 | −0.765 | −1.878 | 4.05 | 2.18 | 2.762 | 1.974 |
| MSTC.MaLR.LTR.3115.(284) | 211 | 0.183 | 0.288 | 4.606 | 1.849 | 2.72 | 1.968 |
| HUERS-P3.ERV1.LTR.841.(806) | 94 | 0.322 | 0.466 | 4.742 | 1.514 | 2.745 | 1.913 |
| nonrep.149154 | 1 | −0.146 | −2.955 | 2.345 | 3.309 | 2.475 | 1.907 |
| nonrep.122203 | 1 | −1.072 | −3.291 | 4.015 | 2.941 | 2.528 | 1.887 |
| gene.39459 | 1 | 0.909 | −1.149 | 1.138 | 3.194 | 2.581 | 1.883 |
| gene.63060 | 1 | −0.415 | −2.355 | 3.746 | 3.155 | 2.508 | 1.874 |
| MER50.ERV1.LTR.2576.(431) | 408 | 0.233 | 0.36 | 4.03 | 2.436 | 2.368 | 1.874 |
| MLT1G.MaLR.LTR.3459.(318) | 453 | 0.09 | 0.172 | 4.247 | 2.137 | 2.53 | 1.848 |
| MLT1A0.MaLR.LTR.20156.(279) | 1043 | 0.114 | 0.218 | 4.584 | 1.589 | 2.482 | 1.825 |
| gene.38409 | 1 | −1.687 | −2.625 | 4.028 | 1.615 | 2.493 | 1.822 |
| MER51B.ERV1.LTR.697.(303) | 90 | 0.163 | 0.289 | 4.171 | 2.587 | 2.399 | 1.815 |
| MLT1E.MaLR.LTR.3378.(359) | 649 | 0.046 | 0.157 | 3.127 | 2.524 | 2.388 | 1.806 |
| nonrep.153897 | 1 | −0.68 | −2.597 | 3.277 | 2.802 | 2.512 | 1.803 |
| gene.248283 | 1 | −1.003 | −2.526 | 3.77 | 2.981 | 2.326 | 1.779 |

Random Forest to Classify Normal, Margin and Tumor (Genes + NonRep. + Rep.[Body Only], 139379 vars)

TABLE 7
Importance of 901 repetitive element and literature based variables.
Repetitive element categories and 5 categories defined based on literature, ranked based on
the decreased mean accuracy in classification using Random Forest. The meanMargin and
meanTumor columns show the methylation level in category among Non-Tumor Adjacent
and Tumor experiments respectively. Columns 1 (Normal), 2 (Non-Tumor Adjacent), and 3
(Tumor) indicate the decrease in the prediction accuracy per category in a given subset of
experiments during the cross validation. MeanDecreaseAccuracy is an average decrease of
overall accuracy of classification. MeanDecreaseGini indicates average decrease in Gini
statistic. The 5 categories defined in the literature are highlighted in bold.

| | | | | | | | | Mean | Mean |
|---|---|---|---|---|---|---|---|---|---|
| | | | mean | mean | | | | Decrease | Decrease |
| rank | description of category | count | Margin | Tumor | 1 | 2 | 3 | Accuracy | Gini |
| 1 | MER67D.ERV1.LTR.1130.[294] | 49 | 0.21 | 0.41 | 17.6 | 5.9 | 15.0 | 10.52 | 1.26 |
| 2 | HUERS- | 28 | 0.29 | 0.52 | 24.9 | 13.0 | 14.2 | 10.30 | 2.04 |
| 3 | MER6.MER2_type.DNA. 926.[41 [symbol] | 1995 | 0.04 | 0.13 | 23.9 | 14.8 | 13.3 | 10.01 | 2.04 |
| 4 | MER66C.ERV1.LTR.661.[279] | 54 | 0.06 | 0.22 | 22.5 | 15.1 | 12.7 | 9.56 | 1.84 |
| 5 | ERVL.ERVL.LTR.826.[422] | 6129 | 0.12 | 0.21 | 18.4 | 10.8 | 9.2 | 7.52 | 1.23 |
| 6 | MLT1G1.MaLR.LTR.4437.[320] | 247 | 0.07 | 0.18 | 12.6 | 8.5 | 7.8 | 6.07 | 0.78 |
| 7 | MER41E- | 1 | 2.73 | 2.40 | 15.0 | 9.8 | −0.9 | 5.79 | 0.43 |
| 8 | MER4D.ERV1.LTR.1275.[421] | 247 | 0.08 | 0.17 | 13.7 | 6.0 | 7.4 | 5.78 | 0.68 |
| 9 | MLT2D.ERVL.LTR.4035.[266] | 174 | 0.12 | 0.22 | 12.1 | 6.8 | 7.5 | 5.68 | 0.68 |
| 10 | Charlie5.MER1_type.DNA.346 | 261 | −0.19 | −0.15 | 14.1 | 8.2 | 0.0 | 5.29 | 0.37 |
| 11 | body.LTR | 38991 | 0.18 | 0.27 | 13.7 | 4.3 | 6.6 | 5.28 | 0.60 |
| 12 | MLT1A1.MaLR.LTR.6611.[294] | 321 | 0.09 | 0.21 | 12.5 | 7.3 | 6.8 | 5.25 | 0.58 |
| 13 | HUERS- | 94 | 0.32 | 0.47 | 12.2 | 2.9 | 6.5 | 5.08 | 0.47 |
| 14 | Cheshire.MER1_type.DNA.747 | 66 | −0.27 | −0.14 | 11.2 | 8.2 | −0.7 | 4.45 | 0.27 |
| 15 | HERVL.ERVL.LTR.1081.[547] | 238 | 0.15 | 0.30 | 7.9 | 2.6 | 5.9 | 4.25 | 0.35 |
| 16 | LTR19C- | 5 | −0.56 | −0.63 | 3.7 | −4.1 | 6.8 | 4.23 | 0.11 |
| 17 | MER70A.ERVL.LTR.524.[386] | 39 | 0.14 | 0.31 | 10.1 | 3.6 | 5.5 | 4.21 | 0.37 |
| 18 | MER50B.ERV1.LTR.681.[468] | 102 | 0.07 | 0.26 | 8.3 | 5.3 | 5.4 | 4.15 | 0.33 |
| 19 | MER51B.ERV1.LTR.697.[303] | 90 | 0.16 | 0.29 | 9.9 | 5.8 | 5.1 | 4.08 | 0.36 |
| 20 | THE1B.MaLR.LTR.22106.[329] | 719 | 0.32 | 0.46 | 11.2 | 2.6 | 4.3 | 4.07 | 0.29 |
| 21 | LTR7A.ERV1.LTR.16.[122] | 4 | 1.32 | 1.30 | 11.0 | 5.9 | −0.6 | 4.07 | 0.22 |
| 22 | MLT2B3.ERVL.LTR.2966.[406] | 132 | 0.24 | 0.42 | 11.0 | 1.5 | 4.8 | 4.03 | 0.32 |
| 23 | MER52C- | 1 | 1.94 | 1.62 | 10.8 | 6.1 | −0.8 | 3.94 | 0.21 |
| 24 | MER11B.ERVK.LTR.552.[932] | 41 | 0.73 | 0.94 | 10.7 | 4.6 | 2.0 | 3.93 | 0.23 |
| 25 | LTR52.ERVL.LTR.1035.[277] | 69 | 0.29 | 0.31 | 10.9 | 4.6 | −0.2 | 3.91 | 0.20 |
| 26 | Charlie3.MER1_type.DNA.639 | 57 | −0.03 | 0.11 | 2.8 | 4.3 | 5.2 | 3.91 | 0.20 |
| 27 | body.MaLR | 15574 | 0.13 | 0.22 | 10.0 | 3.3 | 4.8 | 3.91 | 0.33 |
| 28 | tRNA-Pro- | 3 | 0.28 | 0.19 | 0.9 | 4.8 | 5.2 | 3.83 | 0.12 |
| 29 | THE1- | 14 | 0.85 | 1.25 | 5.9 | 1.5 | 4.7 | 3.79 | 0.14 |
| 30 | LTR10B.ERV1.LTR.70.[433] | 43 | 0.42 | 0.40 | 10.3 | 4.8 | −0.9 | 3.74 | 0.20 |
| 31 | MER50.ERV1.LTR.2576.[431] | 408 | 0.23 | 0.36 | 10.4 | 4.0 | 3.3 | 3.72 | 0.26 |
| 32 | LTR14B.ERVK.LTR.358.[464] | 59 | 0.50 | 0.75 | 10.0 | 4.6 | 0.8 | 3.66 | 0.17 |
| 33 | LTR22.ERVK.LTR.103.[478] | 88 | 0.16 | 0.33 | 8.5 | 5.9 | 3.5 | 3.57 | 0.28 |
| 34 | HUERS- | 32 | 0.14 | 0.27 | 3.8 | 1.5 | 4.8 | 3.53 | 0.13 |
| 35 | MER92B.ERV1.LTR.870.[297] | 72 | 0.36 | 0.38 | 9.4 | 5.1 | −0.3 | 3.45 | 0.16 |
| 36 | LTR9B.ERV1.LTR.745.[398] | 135 | 0.50 | 0.64 | 9.5 | 3.6 | 0.6 | 3.45 | 0.15 |
| 37 | MSTC.MaLR.LTR.3115.[284] | 211 | 0.18 | 0.29 | 9.0 | 2.4 | 4.5 | 3.45 | 0.24 |
| 38 | LTR2.ERV1.LTR.847.[415] | 1075 | 0.23 | 0.34 | 8.6 | 3.5 | 2.6 | 3.44 | 0.16 |
| 39 | LTR21B.ERV1.LTR.58.[342] | 10 | 0.88 | 0.77 | 9.4 | 5.5 | −0.3 | 3.44 | 0.15 |
| 40 | PABL_A.ERV1.LTR.514.[471] | 62 | −0.03 | 0.07 | 0.5 | 3.3 | 4.5 | 3.31 | 0.09 |
| 41 | MSTD.MaLR.LTR.7421.[291] | 365 | 0.25 | 0.36 | 8.9 | 3.7 | 2.0 | 3.28 | 0.17 |
| 42 | LTR77.ERV1.LTR.149.[430] | 7 | 0.53 | 0.78 | 8.4 | 3.4 | 2.6 | 3.24 | 0.22 |
| 43 | LTR33A.ERVL.LTR.3521.[226] | 174 | 0.13 | 0.22 | 5.5 | 1.2 | 4.0 | 3.16 | 0.10 |
| 44 | LTR8.ERV1.LTR.3520.[439] | 486 | 0.27 | 0.36 | 8.7 | 3.5 | 1.6 | 3.15 | 0.12 |
| 45 | HERVFH21.ERV1.LTR.183.[962 [symbol] | 60 | 0.90 | 0.77 | 9.0 | 5.2 | −0.3 | 3.15 | 0.13 |
| 46 | MLT1G.MaLR.LTR.3459.[318] | 453 | 0.09 | 0.17 | 7.6 | 2.2 | 4.2 | 3.14 | 0.16 |
| 47 | MLT2B2.ERVL.LTR.2019.[335] | 91 | −0.07 | 0.10 | −1.3 | 6.3 | 4.2 | 3.13 | 0.22 |
| 48 | LTR38C.ERV1.LTR.132.[373] | 13 | 0.52 | 0.66 | 8.9 | 3.6 | 0.7 | 3.12 | 0.13 |
| 49 | MER11A.ERVK.LTR.930.[920] | 43 | 0.66 | 0.98 | 7.1 | 1.6 | 3.6 | 3.11 | 0.13 |
| 50 | MER66- | 7 | 0.87 | 0.88 | 8.8 | 2.7 | −0.1 | 3.06 | 0.12 |
| 51 | MER70- | 7 | 0.90 | 0.98 | 8.3 | 3.3 | 0.4 | 3.03 | 0.13 |
| 52 | LTR19B- | 4 | 0.76 | 0.72 | 8.5 | 3.3 | 0.3 | 2.99 | 0.12 |
| 53 | LTR8A.ERV1.LTR.2663.[482] | 154 | 0.31 | 0.38 | 8.8 | 2.6 | 1.1 | 2.98 | 0.12 |
| 54 | MER34C.ERV1.LTR.257.[221] | 89 | −0.04 | −0.01 | −0.6 | 0.8 | 4.8 | 2.98 | 0.08 |
| 55 | MER51A.ERV1.LTR.953.[414] | 104 | 0.30 | 0.33 | 8.6 | 2.5 | 0.9 | 2.97 | 0.12 |
| 56 | MER92A.ERV1.LTR.145.[271] | 12 | 0.48 | 0.61 | 8.2 | 3.6 | −0.0 | 2.95 | 0.11 |
| 57 | MER115.Tip100.DNA.2382.[29 [symbol] | 141 | 0.15 | 0.17 | 8.5 | 3.6 | 0.2 | 2.95 | 0.11 |
| 58 | L1PA10.L1.LINE.6962.[920] | 342 | 0.33 | 0.52 | 8.4 | 1.7 | 1.1 | 2.95 | 0.12 |
| 59 | MSTB- | 34 | 0.61 | 0.80 | 8.5 | 2.8 | 1.6 | 2.94 | 0.11 |
| 60 | LTR48.ERV1.LTR.1227.[342] | 127 | 0.27 | 0.33 | 8.1 | 3.8 | −0.1 | 2.90 | 0.10 |
| 61 | MSTB1.MaLR.LTR.4879.[326] | 224 | 0.34 | 0.45 | 8.3 | 3.5 | 1.1 | 2.90 | 0.11 |
| 62 | LTR21A.ERV1.LTR.89.[418] | 19 | 0.98 | 0.95 | 8.5 | 3.5 | −0.1 | 2.88 | 0.11 |
| 63 | MSTA.MaLR.LTR.19102.[319] | 904 | 0.36 | 0.50 | 8.3 | 2.2 | 1.5 | 2.87 | 0.11 |
| 64 | MSTB.MaLR.LTR.8397.[321] | 777 | 0.33 | 0.45 | 8.0 | 2.1 | 1.1 | 2.86 | 0.11 |
| 65 | LOR1a- | 7 | 0.66 | 0.65 | 8.2 | 3.6 | −0.3 | 2.85 | 0.11 |
| 66 | MER83C- | 3 | 1.34 | 1.49 | 8.3 | 2.8 | 0.5 | 2.84 | 0.11 |
| 67 | Kanga1a.Tc2.DNA.310.[392] | 27 | 0.25 | 0.29 | 8.0 | 3.6 | 0.7 | 2.83 | 0.10 |
| 68 | LTR19A.ERV1.LTR.417.[359] | 37 | 0.29 | 0.37 | 8.3 | 2.8 | 0.1 | 2.81 | 0.11 |
| 69 | LTR43- | 9 | −0.31 | −0.18 | 1.1 | 5.2 | 2.7 | 2.81 | 0.10 |
| 70 | body.AcHobo | 997 | 0.02 | 0.08 | 4.3 | 3.6 | 3.7 | 2.81 | 0.14 |
| 71 | LTR36.ERV1.LTR.474.[382] | 50 | 0.18 | 0.30 | 5.2 | 2.1 | 3.6 | 2.81 | 0.17 |
| 72 | MLT1A.MaLR.LTR.8650.[280] | 1783 | 0.11 | 0.21 | 7.1 | 3.7 | 3.2 | 2.79 | 0.17 |
| 73 | MER11D.ERVK.LTR.259.[745] | 37 | 0.68 | 1.04 | 6.3 | 1.5 | 2.1 | 2.78 | 0.11 |
| 74 | body.ERV | 24732 | 0.21 | 0.31 | 6.8 | 3.0 | 1.9 | 2.76 | 0.12 |
| 75 | LTR25- | 13 | 0.51 | 0.60 | 8.0 | 1.8 | 1.2 | 2.76 | 0.11 |
| 76 | LTR25.ERV1.LTR.261.[466] | 28 | 0.50 | 0.57 | 7.8 | 2.4 | −0.7 | 2.74 | 0.11 |
| 77 | THE1C.MaLR.LTR.9731.[329] | 349 | 0.34 | 0.51 | 7.9 | 0.2 | 1.8 | 2.73 | 0.15 |
| 78 | MER4A1.ERV1.LTR.2150.[358] | 257 | 0.18 | 0.29 | 7.5 | 1.3 | 2.7 | 2.69 | 0.13 |
| 79 | LTR5A.ERVK.LTR.250.[947] | 34 | 0.60 | 0.68 | 4.3 | 1.7 | 3.0 | 2.65 | 0.06 |
| 80 | MER4D1.ERV1.LTR.1393.[464] | 110 | 0.17 | 0.25 | 7.2 | 1.2 | 2.4 | 2.61 | 0.12 |
| 81 | HY1.scRNA.scRNA.428.[97] | 43 | −0.20 | −0.13 | 6.9 | 4.2 | −0.9 | 2.53 | 0.09 |
| 82 | THE1A.MaLR.LTR.4161.[319] | 170 | 0.37 | 0.52 | 7.1 | 1.0 | −0.0 | 2.47 | 0.08 |
| 83 | THE1A- | 25 | 0.36 | 0.56 | 7.3 | 1.4 | −0.2 | 2.47 | 0.10 |
| 84 | L1MA7.L1.LINE.7550.[459] | 383 | 0.21 | 0.34 | 6.8 | 1.1 | 2.6 | 2.46 | 0.11 |
| 85 | L1PB4.L1.LINE.6330.[529] | 329 | 0.18 | 0.34 | 6.5 | 1.3 | 3.0 | 2.46 | 0.12 |
| 86 | MLT2B1.ERVL.LTR.4186.[325] | 169 | 0.14 | 0.31 | 6.6 | 2.0 | 3.0 | 2.44 | 0.13 |
| 87 | MLT1E.MaLR.LTR.3378.[359] | 649 | 0.05 | 0.16 | 4.5 | 4.1 | 2.7 | 2.44 | 0.14 |
| 88 | MLT1E2.MaLR.LTR.3960.[377] | 239 | 0.04 | 0.18 | 4.0 | 3.3 | 3.0 | 2.43 | 0.13 |
| 89 | LTR10F.ERV1.LTR.432.[415] | 93 | 0.74 | 0.93 | 6.8 | 1.2 | 0.3 | 2.40 | 0.08 |
| 90 | ERVL- | 133 | 0.10 | 0.19 | 3.6 | 4.8 | 2.4 | 2.40 | 0.15 |
| 91 | HERVL- | 20 | 0.28 | 0.55 | 6.0 | 0.3 | 3.1 | 2.40 | 0.11 |
| 92 | body.ERVK | 1562 | 0.36 | 0.50 | 4.4 | 1.6 | 2.3 | 2.37 | 0.06 |
| 93 | LTR75_1.ERV1.LTR.129.[320] | 14 | −0.16 | −0.01 | −1.3 | 5.6 | 1.9 | 2.36 | 0.07 |
| 94 | PRIMA4_LTR.ERV1.LTR.344.[4 [symbol] | 32 | 0.19 | 0.35 | 6.3 | 1.5 | 2.9 | 2.36 | 0.12 |
| 95 | LTR10D.ERV1.LTR.189.[431] | 31 | 0.43 | 0.42 | 6.2 | 3.0 | 0.4 | 2.36 | 0.08 |
| 96 | LTR1B.ERV1.LTR.1147.[622] | 110 | 0.42 | 0.59 | 5.3 | −0.7 | 2.3 | 2.30 | 0.06 |
| 97 | tRNA-Leu- | 2 | 0.93 | 0.72 | 6.1 | 3.4 | −0.1 | 2.28 | 0.08 |
| 98 | MER69A.Tip100.DNA.1046.[14 [symbol] | 70 | 0.23 | 0.25 | 6.2 | 0.6 | 0.6 | 2.27 | 0.06 |
| 99 | THE1D.MaLR.LTR.12381.[324] | 511 | 0.29 | 0.44 | 6.6 | 0.3 | 1.1 | 2.24 | 0.08 |
| 100 | MER74A.ERVL.LTR.1526.[372] | 116 | 0.33 | 0.30 | 6.0 | 3.2 | −0.7 | 2.24 | 0.07 |
| 101 | LTR14.ERVK.LTR.83.[463] | 127 | 0.45 | 0.63 | 6.6 | 1.6 | 0.5 | 2.24 | 0.06 |
| 102 | HERV16.ERVL.LTR.4493.[505] | 186 | 0.11 | 0.23 | 5.5 | 0.5 | 2.9 | 2.23 | 0.09 |
| 103 | MLT2B4.ERVL.LTR.4120.[305] | 157 | 0.12 | 0.23 | 5.6 | 0.8 | 2.8 | 2.23 | 0.11 |
| 104 | MER9.ERVK.LTR.1128.[475] | 1496 | 0.09 | 0.13 | 5.6 | 1.8 | 1.0 | 2.19 | 0.06 |
| 105 | MADE2.Mariner.DNA.2609.[69 [symbol] | 119 | −0.23 | −0.18 | 5.0 | 3.7 | 0.5 | 2.19 | 0.07 |
| 106 | LTR69.ERVL.LTR.140.[376] | 3 | 0.86 | 0.71 | 6.2 | 2.3 | −0.3 | 2.18 | 0.06 |
| 107 | LTR10B1.ERV1.LTR.242.[314] | 35 | 0.37 | 0.32 | 5.3 | 2.6 | 0.2 | 2.17 | 0.06 |
| 108 | MER45B.Tip100.DNA.1173.[40 [symbol] | 109 | −0.12 | −0.05 | 3.6 | 4.0 | 0.2 | 2.16 | 0.09 |
| 109 | LTR9.ERV1.LTR.1919.[459] | 427 | 0.33 | 0.48 | 5.6 | 0.3 | 1.8 | 2.15 | 0.08 |
| 110 | body.Unknown | 108 | 0.54 | 0.54 | 6.1 | 2.0 | −0.8 | 2.14 | 0.07 |
| 111 | MER11C.ERVK.LTR.825.[998] | 138 | 0.49 | 0.69 | 5.8 | 0.6 | 1.3 | 2.12 | 0.06 |
| 112 | HERVK9.ERVK.LTR.723.[1250] | 77 | 0.90 | 1.22 | 5.6 | 1.0 | 1.5 | 2.09 | 0.06 |
| 113 | body.ERV1 | 17661 | 0.23 | 0.33 | 5.6 | 1.8 | 1.6 | 2.07 | 0.09 |
| 114 | HERVL- | 44 | 0.62 | 0.93 | 3.1 | −0.2 | 2.5 | 2.04 | 0.03 |
| 115 | LTR34- | 1 | −0.86 | −0.59 | 5.2 | 3.0 | −0.3 | 2.01 | 0.10 |
| 116 | MLT- | 139 | 0.46 | 0.63 | 6.2 | −0.9 | 0.5 | 2.00 | 0.06 |
| 117 | body.SVA | 1653 | 0.59 | 0.62 | 5.9 | 0.6 | 0.1 | 1.99 | 0.06 |
| 118 | LTR3.ERVK.LTR.83.[383] | 1388 | 0.15 | 0.23 | 4.5 | 0.6 | 2.1 | 1.97 | 0.05 |
| 119 | MLT1A0.MaLR.LTR.20156.[279 [symbol] | 1043 | 0.11 | 0.22 | 4.9 | 1.6 | 2.2 | 1.95 | 0.07 |
| 120 | MER4E.ERV1.LTR.694.[476] | 158 | 0.18 | 0.22 | 6.2 | −0.4 | −0.0 | 1.94 | 0.07 |
| 121 | L1PA8.L1.LINE.7715.[856] | 479 | 0.33 | 0.52 | 5.8 | −0.0 | 1.0 | 1.94 | 0.04 |
| 122 | MER58B.MER1_type.DNA.6800. [symbol] | 490 | −0.11 | −0.05 | 4.2 | 3.8 | −0.0 | 1.94 | 0.07 |
| 123 | LTR10A.ERV1.LTR.301.[449] | 29 | 0.39 | 0.49 | 4.7 | 1.5 | 1.3 | 1.92 | 0.05 |
| 124 | GSATII.centr.Satellite.157 [symbol] | 73 | −0.34 | −0.31 | 5.9 | −0.1 | −0.2 | 1.92 | 0.06 |
| 125 | Tigger2a.MER2_type.DNA.320 [symbol] | 218 | 0.19 | 0.26 | 5.1 | 1.2 | −0.2 | 1.92 | 0.05 |
| 126 | MLT2A1.ERVL.LTR.3220.[327] | 97 | 0.42 | 0.56 | 5.8 | −0.1 | −0.5 | 1.91 | 0.06 |
| 127 | body.rRNA | 107 | 0.16 | 0.16 | 4.9 | 2.5 | −0.4 | 1.90 | 0.05 |
| 128 | MER61A.ERV1.LTR.791.[303] | 111 | 0.14 | 0.26 | 4.1 | 2.5 | 1.0 | 1.89 | 0.05 |
| 129 | MER74B.ERVL.LTR.936.[328] | 80 | 0.27 | 0.32 | 5.8 | −0.9 | 0.5 | 1.89 | 0.06 |
| 130 | body.Other | 1653 | 0.59 | 0.62 | 5.7 | −0.2 | 0.4 | 1.88 | 0.05 |
| 131 | LTR7B.ERV1.LTR.861.[364] | 137 | 0.42 | 0.57 | 4.9 | 1.7 | 0.9 | 1.88 | 0.04 |
| 132 | MER66A- | 3 | −0.38 | 0.02 | 1.1 | 2.6 | 1.9 | 1.87 | 0.05 |
| 133 | FordPrefect.Tip100.DNA.410 [symbol] | 45 | −0.13 | −0.10 | 4.2 | 3.0 | −0.2 | 1.87 | 0.06 |
| 134 | MLT1F.MaLR.LTR.4498.[327] | 726 | 0.03 | 0.12 | 2.9 | 2.4 | 2.4 | 1.87 | 0.07 |
| 135 | LTR54B.ERV1.LTR.832.[289] | 53 | −0.05 | 0.09 | 0.7 | 1.7 | 2.4 | 1.86 | 0.05 |
| 136 | MER41A.ERV1.LTR.2479.[384] | 246 | 0.06 | 0.17 | 3.2 | 2.8 | 2.0 | 1.85 | 0.08 |
| 137 | THE1B- | 46 | 0.39 | 0.64 | 4.8 | 0.5 | 1.3 | 1.84 | 0.04 |
| 138 | L1MA1.L1.LINE.3940.[1012] | 494 | 0.11 | 0.27 | 4.8 | 1.7 | 2.2 | 1.83 | 0.07 |
| 139 | LTR14A.ERVK.LTR.113.[329] | 19 | 0.87 | 1.07 | 4.6 | 1.5 | 1.1 | 1.83 | 0.05 |
| 140 | 5S.rRNA.rRNA.1258.[70] | 107 | 0.16 | 0.16 | 4.8 | 2.5 | −0.2 | 1.83 | 0.05 |
| 141 | LTR16A1.ERVL.LTR.3898.[252 [symbol] | 197 | 0.13 | 0.19 | 4.0 | 2.7 | 0.9 | 1.83 | 0.04 |
| 142 | HSMAR1.Mariner.DNA.703.[66 [symbol] | 100 | 0.19 | 0.46 | 4.3 | 2.8 | 1.9 | 1.81 | 0.07 |
| 143 | MER73.ERVL.LTR.270.[408] | 22 | 0.46 | 0.53 | 5.6 | −1.9 | 0.7 | 1.80 | 0.05 |
| 144 | LTR16A.ERVL.LTR.6873.[276] | 510 | 0.14 | 0.21 | 5.1 | −0.3 | 1.4 | 1.79 | 0.05 |
| 145 | MER110.ERV1.LTR.736.[250] | 83 | 0.06 | 0.17 | 3.1 | 1.3 | 2.5 | 1.79 | 0.07 |
| 146 | LTR5B.ERVK.LTR.420.[781] | 111 | 0.44 | 0.51 | 3.4 | 0.5 | 1.8 | 1.77 | 0.04 |
| 147 | LTR7Y.ERV1.LTR.472.[361] | 102 | 0.34 | 0.56 | 2.8 | −0.8 | 2.4 | 1.77 | 0.03 |
| 148 | ERVL- | 22 | 0.29 | 0.41 | 4.9 | 0.1 | 0.9 | 1.76 | 0.04 |
| 149 | LOR1a.ERV1.LTR.789.[380] | 28 | 0.39 | 0.45 | 5.2 | 1.0 | −0.9 | 1.75 | 0.05 |
| 150 | LTR41.ERVL.LTR.2357.[234] | 134 | 0.05 | 0.14 | 1.1 | 1.3 | 2.5 | 1.74 | 0.02 |
| 151 | LTR5.ERVK.LTR.23.[192] | 851 | 0.14 | 0.22 | 3.8 | −0.3 | 1.9 | 1.74 | 0.03 |
| 152 | PABL_B.ERV1.LTR.361.[373] | 36 | 0.26 | 0.29 | 4.2 | 1.3 | 0.6 | 1.74 | 0.04 |
| 153 | LTR12B.ERV1.LTR.450.[875] | 80 | 0.47 | 0.62 | 4.9 | 0.9 | 1.0 | 1.74 | 0.05 |
| 154 | LTR22A.ERVK.LTR.187.[402] | 15 | 0.09 | 0.28 | 1.5 | 2.8 | 1.9 | 1.72 | 0.04 |
| 155 | MLT1E- | 5 | −0.00 | −0.14 | −0.5 | 1.4 | 2.6 | 1.72 | 0.02 |
| 156 | LTR38.ERV1.LTR.313.[381] | 61 | 0.19 | 0.31 | 4.3 | −0.9 | 1.4 | 1.71 | 0.03 |
| 157 | HERVI.ERV1.LTR.124.[2141] | 181 | 0.39 | 0.50 | 4.2 | 0.6 | 1.3 | 1.68 | 0.04 |
| 158 | MLT2A2.ERVL.LTR.3754.[411] | 136 | 0.31 | 0.52 | 4.3 | 0.6 | 1.6 | 1.68 | 0.05 |
| 159 | MLT1J.MaLR.LTR.15375.[246] | 1277 | 0.02 | 0.09 | 2.9 | 1.9 | 2.4 | 1.66 | 0.06 |
| 160 | MER66C- | 10 | 0.22 | 0.50 | 3.8 | 0.3 | 2.0 | 1.65 | 0.05 |
| 161 | HERVP71A.ERV1.LTR.190.[134 [symbol] | 38 | 0.40 | 0.51 | 4.5 | 1.1 | −0.4 | 1.64 | 0.04 |
| 162 | L1M2b.L1.LINE.318.[858] | 12 | 0.38 | 0.52 | 4.4 | 0.2 | 0.7 | 1.61 | 0.03 |
| 163 | L1PA11.L1.LINE.3886.[997] | 193 | 0.40 | 0.64 | 4.5 | −1.0 | 0.7 | 1.59 | 0.04 |
| 164 | MER31A.ERV1.LTR.1316.[321] | 110 | 0.14 | 0.13 | 3.4 | 2.3 | 0.4 | 1.59 | 0.04 |
| 165 | LTR22C.ERVK.LTR.378.[442] | 41 | 0.24 | 0.43 | 4.5 | 0.7 | 1.7 | 1.58 | 0.07 |
| 166 | MLT2F.ERVL.LTR.1917.[323] | 114 | 0.11 | 0.19 | 4.3 | 1.4 | 1.5 | 1.58 | 0.05 |
| 167 | LTR38B- | 2 | 1.18 | 1.29 | 4.5 | −0.3 | 0.3 | 1.57 | 0.03 |
| 168 | MLT1E2- | 2 | −0.93 | −0.51 | 3.3 | 2.5 | 0.9 | 1.57 | 0.04 |
| 169 | MER63C.AcHobo.DNA.602.[467 [symbol] | 56 | −0.21 | −0.13 | 2.8 | 2.9 | 0.0 | 1.56 | 0.04 |
| 170 | LTR1D.ERV1.LTR.978.[707] | 98 | 0.39 | 0.54 | 5.1 | −0.8 | 1.4 | 1.56 | 0.05 |
| 171 | LTR1.ERV1.LTR.1396.[612] | 6740 | 0.33 | 0.42 | 4.7 | 0.5 | 0.6 | 1.55 | 0.04 |
| 172 | MER34D.ERV1.LTR.339.[322] | 28 | 0.41 | 0.37 | 4.0 | 1.3 | −0.2 | 1.55 | 0.04 |
| 173 | MER63A.AcHobo.DNA.2791.[18 [symbol] | 127 | 0.06 | 0.16 | 2.8 | 1.3 | 2.1 | 1.55 | 0.04 |
| 174 | THE1D- | 27 | 0.55 | 0.73 | 4.4 | −0.5 | 0.4 | 1.55 | 0.03 |
| 175 | LTR3A.ERVK.LTR.158.[396] | 37 | 0.43 | 0.58 | 3.7 | 0.2 | 1.2 | 1.55 | 0.03 |
| 176 | body.snRNA | 296 | −0.11 | −0.10 | 4.2 | 1.8 | −0.3 | 1.54 | 0.06 |
| 177 | MER39.ERV1.LTR.3125.[336] | 323 | 0.04 | 0.13 | 3.5 | 1.6 | 2.0 | 1.53 | 0.04 |
| 178 | MER4B.ERV1.LTR.1709.[402] | 196 | 0.04 | 0.14 | 3.3 | 0.9 | 1.9 | 1.53 | 0.04 |
| 179 | MER63.AcHobo.DNA.3400.[139 [symbol] | 500 | −0.03 | 0.04 | −1.0 | 3.2 | 1.5 | 1.53 | 0.02 |
| 180 | MER91C.Tip100.DNA.1249.[92 [symbol] | 44 | −0.20 | −0.16 | 4.0 | 1.9 | −0.4 | 1.52 | 0.04 |
| 181 | MER41B.ERV1.LTR.2864.[370] | 267 | −0.03 | 0.08 | −0.8 | 3.3 | 0.6 | 1.52 | 0.02 |
| 182 | HERVK.SVA, ERVK.Other, LTR.3 [symbol] | 454 | 0.36 | 0.53 | 3.3 | 0.4 | 1.2 | 1.52 | 0.02 |
| 183 | LTR67.ERVL.LTR.6289.[175] | 286 | 0.08 | 0.15 | 3.5 | −0.5 | 1.9 | 1.50 | 0.03 |
| 184 | MER61E.ERV1.LTR.308.[387] | 18 | 0.39 | 0.51 | 4.4 | −0.2 | 0.2 | 1.49 | 0.03 |
| 185 | ERVL-D.ERVL.LTR.249.[796] | 11 | 0.15 | 0.39 | 3.3 | 1.6 | 1.7 | 1.48 | 0.06 |
| 186 | L1MDb.L1.LINE.1862.[510] | 135 | 0.19 | 0.28 | 4.3 | 1.0 | −0.5 | 1.48 | 0.03 |
| 187 | U1.snRNA.snRNA.183.[129] | 61 | −0.12 | −0.27 | 1.9 | −0.0 | 2.3 | 1.47 | 0.02 |
| 188 | HERV17.ERV1.LTR.919.[982] | 107 | 0.77 | 0.92 | 4.2 | 0.1 | 0.7 | 1.45 | 0.03 |
| 189 | body.LINE | 79198 | 0.08 | 0.17 | 3.9 | 0.5 | 1.6 | 1.43 | 0.03 |
| 190 | L1MA9.L1.LINE.14394.[495] | 729 | 0.17 | 0.33 | 4.6 | −0.4 | 1.4 | 1.43 | 0.04 |
| 191 | MSTD- | 34 | 0.37 | 0.63 | 3.7 | 0.6 | 0.8 | 1.42 | 0.02 |
| 192 | LTR33.ERVL.LTR.9302.[253] | 659 | 0.17 | 0.24 | 4.0 | −0.2 | 0.9 | 1.42 | 0.03 |
| 193 | L1MB1.L1.LINE.5670.[521] | 300 | 0.12 | 0.29 | 3.5 | −0.0 | 1.7 | 1.42 | 0.03 |
| 194 | MLT1H1.MaLR.LTR.3868.[316] | 216 | 0.02 | 0.09 | 0.6 | 1.0 | 2.0 | 1.40 | 0.02 |
| 195 | MER61A- | 48 | 0.21 | 0.28 | 4.1 | 0.8 | −0.0 | 1.40 | 0.03 |
| 196 | L1MA8.L1.LINE.9992.[497] | 560 | 0.16 | 0.30 | 4.2 | −0.9 | 1.5 | 1.40 | 0.04 |
| 197 | MSTA- | 51 | 0.39 | 0.57 | 3.3 | −0.3 | 1.1 | 1.40 | 0.02 |
| 198 | MER41C.ERV1.LTR.799.[320] | 90 | 0.01 | 0.12 | 0.5 | 3.3 | 0.8 | 1.39 | 0.04 |
| 199 | MLT1C- | 17 | −0.00 | 0.15 | −0.6 | 0.0 | 2.3 | 1.39 | 0.04 |
| 200 | MER4A.ERV1.LTR.1084.[386] | 363 | 0.13 | 0.23 | 4.1 | −0.3 | 0.9 | 1.39 | 0.03 |
| 201 | tRNA-Ile- | 10 | 0.09 | −0.16 | 0.1 | 0.7 | 2.0 | 1.38 | 0.02 |
| 202 | PRIMA41- | 13 | 0.39 | 0.50 | 4.1 | −0.2 | 0.0 | 1.37 | 0.03 |
| 203 | MER1A.MER1_type.DNA.2968.[ [symbol] | 299 | 0.13 | 0.17 | 1.8 | −0.0 | 1.7 | 1.37 | 0.01 |
| 204 | PRIMA4- | 160 | 0.16 | 0.21 | 3.3 | −0.1 | 0.8 | 1.36 | 0.03 |
| 205 | MLT1H- | 151 | 0.23 | 0.32 | 4.6 | 0.7 | −0.3 | 1.36 | 0.04 |
| 206 | MSR1.Satellite.Satellite.3 [symbol] | 118 | 0.29 | 0.04 | 3.0 | 2.0 | 0.3 | 1.36 | 0.02 |
| 207 | MER94.AcHobo.DNA.4680.[105 [symbol] | 199 | 0.09 | 0.15 | 3.4 | −0.6 | 1.3 | 1.34 | 0.03 |
| 208 | LTR7.ERV1.LTR.2349.[407] | 935 | 0.29 | 0.48 | 2.9 | −0.7 | 1.4 | 1.34 | 0.02 |
| 209 | MADE1.Mariner.DNA.7773.[61 [symbol] | 348 | −0.00 | 0.12 | 1.4 | 1.4 | 1.7 | 1.34 | 0.03 |
| 210 | LOR1- | 38 | 0.09 | 0.23 | 3.0 | 1.6 | 1.0 | 1.34 | 0.02 |
| 211 | MER83.ERV1.LTR.435.[329] | 77 | 0.14 | 0.23 | 1.9 | −0.4 | 2.0 | 1.33 | 0.02 |
| 212 | MLT1B- | 13 | −0.41 | −0.22 | 2.5 | 2.6 | −0.3 | 1.33 | 0.03 |
| 213 | L1MC4_3endX.L1.LINE.1031.[ [symbol] | 73 | −0.03 | 0.04 | 0.1 | 3.0 | 0.2 | 1.32 | 0.01 |
| 214 | Tigger6b.MER2_type.DNA.96. [symbol] | 4 | 1.17 | 1.30 | 4.6 | −0.6 | −0.7 | 1.32 | 0.04 |
| 215 | LTR76.ERV1.LTR.186.[480] | 17 | 0.46 | 0.73 | 2.9 | −0.3 | 1.6 | 1.31 | 0.02 |
| 216 | L1PA7.L1.LINE.12334.[1556] | 507 | 0.31 | 0.59 | 3.6 | −0.3 | 1.0 | 1.31 | 0.02 |
| 217 | tRNA-His- | 10 | 0.63 | −0.03 | −0.1 | 1.6 | 1.8 | 1.31 | 0.02 |
| 218 | LTR40a.ERVL.LTR.2060.[328] | 108 | −0.01 | 0.16 | 2.4 | 0.6 | 1.7 | 1.30 | 0.04 |
| 219 | MLT1F- | 53 | 0.05 | 0.23 | 2.9 | 2.2 | 1.4 | 1.30 | 0.04 |
| 220 | L1P3b.L1.LINE.165.[1351] | 14 | 0.42 | 0.62 | 3.8 | 0.3 | 0.7 | 1.30 | 0.02 |
| 221 | LTR27.ERV1.LTR.403.[558] | 86 | 0.08 | 0.20 | 2.5 | 2.1 | 1.3 | 1.30 | 0.04 |
| 222 | MSTB2.MaLR.LTR.1595.[329] | 64 | 0.22 | 0.37 | 3.1 | 0.4 | 1.1 | 1.29 | 0.02 |
| 223 | MER67A- | 2 | 0.70 | 0.66 | 4.0 | −1.2 | 0.3 | 1.29 | 0.03 |
| 224 | MER7C.MER2_type.DNA.588.[3 [symbol] | 49 | 0.09 | 0.14 | 1.4 | −1.1 | 2.0 | 1.28 | 0.02 |
| 225 | MLT1D.MaLR.LTR.19943.[325] | 1355 | 0.11 | 0.17 | 2.2 | 1.4 | 1.0 | 1.25 | 0.02 |
| 226 | tRNA-Ser- | 5 | 0.41 | 0.33 | 0.9 | −2.4 | 2.5 | 1.24 | 0.05 |
| 227 | MER92C.ERV1.LTR.334.[159] | 15 | 0.33 | 0.46 | 3.2 | 0.8 | 0.4 | 1.24 | 0.01 |
| 228 | MLT1A1- | 19 | 0.29 | 0.45 | 3.4 | −0.3 | 1.1 | 1.24 | 0.02 |
| 229 | MER76.ERVL.LTR.674.[392] | 50 | 0.13 | 0.24 | 2.6 | 0.8 | 1.0 | 1.24 | 0.02 |
| 230 | MER67A.ERV1.LTR.484.[249] | 39 | 0.12 | 0.21 | 2.0 | 1.2 | 1.2 | 1.24 | 0.01 |
| 231 | HERVIP10F.ERV1.LTR.318.[14 [symbol] | 151 | 0.39 | 0.50 | 3.2 | −0.3 | 0.9 | 1.23 | 0.02 |
| 232 | L1PA13.L1.LINE.10722.[840] | 513 | 0.33 | 0.57 | 3.5 | 0.1 | 0.9 | 1.23 | 0.02 |
| 233 | MLT1E3.MaLR.LTR.1701.[338] | 99 | 0.07 | 0.13 | 3.6 | −0.5 | 1.5 | 1.23 | 0.03 |
| 234 | MLT1A- | 12 | 0.36 | 0.58 | 3.3 | −0.1 | 1.2 | 1.22 | 0.02 |
| 235 | MLT2B5.ERVL.LTR.461.[240] | 16 | 0.36 | 0.47 | 4.0 | −0.7 | 0.3 | 1.22 | 0.03 |
| 236 | LTR19B.ERV1.LTR.277.[423] | 25 | 0.34 | 0.43 | 3.6 | 0.2 | 0.3 | 1.21 | 0.03 |
| 237 | LTR46.ERV1.LTR.231.[325] | 39 | 0.24 | 0.40 | 3.2 | −1.2 | 1.1 | 1.21 | 0.02 |
| 238 | HERV9.ERV1.LTR.1084.[1506] | 148 | 0.90 | 1.06 | 3.2 | −0.5 | 1.3 | 1.21 | 0.02 |
| 239 | LTR28.ERV1.LTR.513.[593] | 30 | 0.24 | 0.28 | 2.9 | 1.2 | 0.2 | 1.21 | 0.02 |
| 240 | LTR47B.ERVL.LTR.380.[336] | 27 | −0.14 | −0.16 | 0.7 | 0.0 | 2.0 | 1.21 | 0.01 |
| 241 | MER4E1.ERV1.LTR.1057.[396] | 102 | 0.19 | 0.22 | 3.5 | −0.6 | −0.2 | 1.20 | 0.02 |
| 242 | MER57A.ERV1.LTR.2633.[290] | 293 | −0.01 | 0.11 | 1.0 | 1.2 | 1.6 | 1.19 | 0.04 |
| 243 | HUERS- | 42 | 0.32 | 0.45 | 3.0 | 0.5 | 0.9 | 1.19 | 0.02 |
| 244 | MER39- | 8 | 0.23 | 0.30 | 1.4 | −0.8 | 1.5 | 1.18 | 0.02 |
| 245 | L1MB3.L1.LINE.15139.[387] | 1556 | 0.09 | 0.12 | 3.2 | 0.1 | 0.4 | 1.18 | 0.02 |
| 246 | LTR1C.ERV1.LTR.242.[459] | 32 | 0.35 | 0.47 | 3.1 | 0.1 | 0.4 | 1.16 | 0.01 |
| 247 | tRNA-Pro- | 2 | 0.26 | −0.53 | 0.1 | 1.5 | 1.6 | 1.16 | 0.01 |
| 248 | LTR32.ERVL.LTR.1289.[281] | 56 | 0.20 | 0.25 | 1.6 | 1.9 | 0.8 | 1.16 | 0.01 |
| 249 | SST1.centr.Satellite.565.[ [symbol] | 126 | 0.05 | −0.19 | −0.6 | 1.4 | 1.5 | 1.16 | 0.01 |
| 250 | PABL_B- | 11 | 0.45 | 0.41 | 3.5 | 0.0 | 0.1 | 1.15 | 0.02 |
| 251 | L1PA12.L1.LINE.2320.[1117] | 136 | 0.37 | 0.62 | 3.3 | −0.7 | 0.7 | 1.15 | 0.01 |
| 252 | HERVIP10FH.ERV1.LTR.594.[1 [symbol] | 87 | 0.52 | 0.60 | 2.2 | 0.2 | 1.0 | 1.15 | 0.01 |
| 253 | MER63D.AcHobo.DNA.1023.[20 [symbol] | 70 | −0.18 | −0.13 | 2.9 | 1.3 | −0.2 | 1.14 | 0.03 |
| 254 | L1MC1.L1.LINE.11868.[608] | 888 | 0.13 | 0.26 | 3.4 | −0.5 | 0.8 | 1.14 | 0.02 |
| 255 | MER39B.ERV1.LTR.1166.[382] | 102 | 0.04 | 0.13 | 2.7 | 0.8 | 1.5 | 1.14 | 0.03 |
| 256 | L1PA6.L1.LINE.5617.[1926] | 291 | 0.32 | 0.55 | 3.0 | −0.5 | 0.9 | 1.13 | 0.01 |
| 257 | MLT1F1.MaLR.LTR.3489.[292] | 204 | −0.05 | 0.05 | −0.0 | 2.2 | 1.1 | 1.13 | 0.04 |
| 258 | tRNA-Gly- | 7 | −0.32 | −0.44 | 3.8 | −0.3 | 0.3 | 1.13 | 0.03 |
| 259 | LTR48B.ERV1.LTR.1058.[346] | 56 | 0.23 | 0.28 | 3.4 | −0.1 | −0.0 | 1.12 | 0.02 |
| 260 | L1PA17.L1.LINE.4722.[700] | 288 | 0.15 | 0.31 | 2.8 | −0.4 | 1.0 | 1.12 | 0.01 |
| 261 | U13_.snRNA.snRNA.304.[98] | 26 | −0.19 | −0.15 | 3.0 | 1.2 | −0.3 | 1.11 | 0.02 |
| 262 | MER54A.ERVL.LTR.626.[458] | 59 | 0.16 | 0.23 | 3.0 | −0.3 | 0.5 | 1.11 | 0.02 |
| 263 | MLT1I.MaLR.LTR.11191.[218] | 507 | 0.07 | 0.17 | 1.5 | 1.1 | 1.3 | 1.11 | 0.01 |
| 264 | L2.L2.LINE.403973.[230] | 24844 | 0.13 | 0.15 | 2.7 | 0.9 | −0.2 | 1.10 | 0.02 |
| 265 | LTR43.ERV1.LTR.436.[329] | 72 | −0.03 | 0.06 | 0.6 | 2.0 | 1.2 | 1.10 | 0.01 |
| 266 | LTR17.ERV1.LTR.830.[578] | 343 | 0.78 | 0.95 | 3.1 | −0.6 | 0.2 | 1.09 | 0.02 |
| 267 | HERVK11.ERVK.LTR.300.[1278 [symbol] | 39 | 0.61 | 0.91 | 2.2 | 0.7 | 0.8 | 1.09 | 0.02 |
| 268 | MER89.ERV1.LTR.1339.[310] | 103 | 0.20 | 0.19 | 2.8 | 0.7 | 0.2 | 1.09 | 0.02 |
| 269 | L1PA5.L1.LINE.10904.[1449] | 547 | 0.26 | 0.51 | 2.3 | 0.5 | 0.8 | 1.09 | 0.01 |
| 270 | U3.snRNA.snRNA.228.[102] | 13 | −0.28 | −0.38 | 3.1 | −1.0 | 1.0 | 1.09 | 0.02 |
| 271 | LTR34.ERV1.LTR.361.[370] | 41 | 0.26 | 0.25 | 3.0 | −0.2 | 0.1 | 1.08 | 0.02 |
| 272 | LTR27B- | 5 | 0.42 | 0.76 | 2.0 | 0.4 | 1.0 | 1.07 | 0.02 |
| 273 | MER8.MER2_type.DNA.1884.[1 [symbol] | 873 | 0.02 | 0.08 | 0.8 | 0.5 | 1.6 | 1.07 | 0.01 |
| 274 | LTR26.ERV1.LTR.604.[405] | 121 | 0.14 | 0.16 | 3.0 | 0.9 | −0.4 | 1.07 | 0.02 |
| 275 | HERVL74.ERVL.LTR.473.[425] | 25 | 0.20 | 0.27 | 2.6 | 0.5 | 0.4 | 1.06 | 0.01 |
| 276 | MER91A.Tip100.DNA.3430.[12 [symbol] | 216 | 0.14 | 0.11 | 3.2 | 0.8 | −0.4 | 1.06 | 0.02 |
| 277 | LTR36- | 5 | −0.30 | 0.04 | 0.1 | 1.7 | 1.2 | 1.06 | 0.02 |
| 278 | HERV3.ERV1.LTR.256.[1560] | 96 | 0.05 | 0.10 | −0.1 | −2.2 | 2.1 | 1.05 | 0.02 |
| 279 | MER61D- | 8 | 0.52 | 0.84 | 2.4 | −0.3 | 0.9 | 1.05 | 0.01 |
| 280 | tRNA-Lys- | 11 | −0.36 | −0.39 | 2.1 | 0.8 | 0.1 | 1.05 | 0.02 |
| 281 | LTR73.ERV1.LTR.205.[393] | 17 | 0.37 | 0.56 | 2.6 | −0.1 | 0.7 | 1.03 | 0.01 |
| 282 | MER72B.ERV1.LTR.177.[394] | 11 | 0.09 | 0.29 | 1.1 | 1.6 | 0.7 | 1.03 | 0.01 |
| 283 | L1P2.L1.LINE.2017.[1383] | 105 | 0.27 | 0.58 | 2.5 | −0.1 | 0.9 | 1.03 | 0.01 |
| 284 | L1PA16.L1.LINE.13098.[813] | 663 | 0.28 | 0.50 | 3.5 | −0.6 | 0.5 | 1.03 | 0.02 |
| 285 | L1M3b.L1.LINE.813.[836] | 35 | 0.36 | 0.61 | 3.2 | −1.0 | 0.8 | 1.02 | 0.01 |
| 286 | L1PBa1.L1.LINE.656.[1545] | 48 | 0.47 | 0.72 | 2.1 | 0.3 | 0.6 | 1.02 | 0.01 |
| 287 | L1P1.L1.LINE.2860.[838] | 183 | 0.27 | 0.59 | 2.6 | 0.0 | 0.8 | 1.02 | 0.01 |
| 288 | Tigger1.MER2_type.DNA.1192 [symbol] | 902 | 0.14 | 0.29 | 3.2 | 0.2 | 1.1 | 1.02 | 0.02 |
| 289 | MER21C.ERV1.LTR.4871.[411] | 362 | 0.05 | 0.11 | 1.1 | 1.0 | 1.3 | 1.02 | 0.01 |
| 290 | LTR19A- | 13 | 0.11 | 0.30 | 0.8 | 0.5 | 1.5 | 1.01 | 0.01 |
| 291 | L1MA5.L1.LINE.4086.[656] | 425 | 0.16 | 0.33 | 3.0 | −1.0 | 0.3 | 1.01 | 0.01 |
| 292 | tRNA-Met.tRNA.tRNA.48.[57] | 6 | −0.14 | −0.36 | 0.7 | 1.9 | 0.9 | 1.01 | 0.01 |
| 293 | L1P.L1.LINE.1589.[520] | 9723 | 0.23 | 0.49 | 2.9 | −1.0 | 1.0 | 1.01 | 0.01 |
| 294 | U13.snRNA.snRNA.178.[98] | 35 | −0.27 | −0.24 | 3.8 | −0.8 | 0.1 | 1.00 | 0.03 |
| 295 | MER44A.MER2_type.DNA.2119. [symbol] | 117 | −0.21 | −0.11 | 2.2 | 1.5 | 0.2 | 0.99 | 0.02 |
| 296 | MER110A.ERV1.LTR.644.[254] | 41 | 0.13 | 0.21 | 1.9 | 1.3 | 0.3 | 0.99 | 0.01 |
| 297 | MER93a.ERV1.LTR.311.[247] | 55 | −0.19 | −0.19 | 3.3 | 0.7 | −0.1 | 0.98 | 0.03 |
| 298 | MER74C.ERVL.LTR.226.[323] | 9 | 0.02 | 0.36 | 0.6 | 1.1 | 1.2 | 0.98 | 0.00 |
| 299 | MLT1H.MaLR.LTR.9741.[277] | 969 | 0.07 | 0.15 | 1.7 | 0.9 | 1.1 | 0.98 | 0.01 |
| 300 | LTR29.ERV1.LTR.667.[307] | 54 | 0.07 | 0.18 | 2.4 | 0.7 | 1.2 | 0.97 | 0.03 |
| 301 | MLT2C2.ERVL.LTR.1282.[249] | 63 | 0.19 | 0.27 | 3.0 | 0.0 | 0.9 | 0.97 | 0.02 |
| 302 | MER6A.MER2_type.DNA.805.[4 [symbol] | 85 | 0.07 | 0.14 | 1.0 | 0.4 | 1.1 | 0.97 | 0.01 |
| 303 | MST-int.MaLR.LTR.563.[425] | 13 | 0.25 | 0.43 | 2.1 | 0.4 | 0.7 | 0.95 | 0.00 |
| 304 | LTR14C.ERVK.LTR.138.[512] | 30 | 0.40 | 0.57 | 2.0 | 0.0 | 0.8 | 0.95 | 0.01 |
| 305 | MLT1B.MaLR.LTR.17353.[278] | 1167 | 0.07 | 0.14 | 2.0 | 0.2 | 1.1 | 0.95 | 0.01 |
| 306 | L1PBb.L1.LINE.271.[1050] | 14 | 0.12 | 0.32 | 1.5 | 0.2 | 1.2 | 0.94 | 0.03 |
| 307 | X. Rago | 494 | −0.26 | 0.06 | 1.5 | 1.0 | 0.8 | 0.94 | 0.01 |
| 308 | AluYh.Alu.SINE.23.[120] | 65 | 0.19 | 0.26 | 2.5 | 0.1 | 0.2 | 0.93 | 0.01 |
| 309 | HERVH48.ERV1.LTR.115.[1792 [symbol] | 58 | 0.62 | 0.67 | 2.4 | 0.7 | −0.1 | 0.93 | 0.01 |
| 310 | Tigger5.MER2_type.DNA.2895 [symbol] | 365 | −0.12 | −0.04 | 1.5 | 1.8 | 0.1 | 0.93 | 0.01 |
| 311 | L1PA3.L1.LINE.10281.[1836] | 1276 | 0.18 | 0.53 | 1.8 | 0.6 | 0.7 | 0.93 | 0.00 |
| 312 | MER9B.ERVK.LTR.61.[431] | 11 | 0.25 | 0.41 | 1.8 | −0.1 | 1.2 | 0.93 | 0.01 |
| 313 | Charlie8.MER1_type.DNA.758 [symbol] | 569 | 0.07 | 0.08 | 2.2 | 0.3 | 0.3 | 0.93 | 0.00 |
| 314 | LTR46- | 7 | 0.44 | 0.50 | 0.7 | −0.1 | 1.3 | 0.92 | 0.02 |
| 315 | L1MC3.L1.LINE.12792.[547] | 881 | −0.01 | 0.13 | 1.4 | 0.0 | 1.3 | 0.92 | 0.02 |
| 316 | MLT1J1.MaLR.LTR.3407.[217] | 142 | 0.07 | 0.15 | 2.1 | 0.1 | 0.8 | 0.92 | 0.01 |
| 317 | HERVK3.ERVK.LTR.341.[1001] | 46 | 0.01 | 0.15 | 1.6 | 0.6 | 1.0 | 0.92 | 0.01 |
| 318 | K. Sunami | 749 | −0.29 | 0.06 | 1.9 | 0.6 | 0.6 | 0.92 | 0.00 |
| 319 | L1M3d.L1.LINE.765.[649] | 54 | 0.13 | 0.34 | 2.6 | −0.4 | 1.0 | 0.91 | 0.01 |
| 320 | MER34.ERV1.LTR.2364.[259] | 419 | 0.04 | 0.11 | 2.1 | 0.9 | 0.6 | 0.91 | 0.01 |
| 321 | LTR31.ERV1.LTR.249.[407] | 19 | 0.32 | 0.32 | 2.7 | 0.7 | 0.4 | 0.91 | 0.02 |
| 322 | MER45C.Tip100.DNA.1080.[29 [symbol] | 62 | 0.14 | 0.11 | 2.5 | 1.1 | −0.3 | 0.91 | 0.01 |
| 323 | L1PA8A.L1.LINE.2210.[1386] | 134 | 0.27 | 0.51 | 3.2 | −1.2 | 0.5 | 0.90 | 0.02 |
| 324 | L1MA6.L1.LINE.4693.[601] | 247 | 0.12 | 0.31 | 1.8 | −0.3 | 0.8 | 0.90 | 0.00 |
| 325 | MER30.MER1_type.DNA.3898.[ [symbol] | 304 | −0.08 | −0.01 | 1.8 | 1.6 | −0.6 | 0.90 | 0.01 |
| 326 | HERVK14.ERVK.LTR.297.[755] | 64 | 0.27 | 0.40 | 2.1 | 0.2 | 0.5 | 0.90 | 0.01 |
| 327 | MER21B.ERV1.LTR.3565.[442] | 275 | 0.01 | 0.06 | −0.9 | 1.2 | 1.2 | 0.89 | 0.01 |
| 328 | L1MA2.L1.LINE.6911.[992] | 385 | 0.22 | 0.43 | 2.0 | −0.1 | 0.9 | 0.89 | 0.00 |
| 329 | LTR49.ERV1.LTR.1406.[325] | 164 | 0.09 | 0.18 | 2.3 | −0.2 | 0.9 | 0.89 | 0.01 |
| 330 | MER21- | 33 | 0.21 | 0.18 | 3.6 | −0.9 | −0.5 | 0.89 | 0.03 |
| 331 | MLT1J2.MaLR.LTR.7867.[215] | 354 | 0.02 | 0.09 | 1.1 | 0.2 | 1.2 | 0.89 | 0.01 |
| 332 | MER46A.MER2_type.DNA.3411. [symbol] | 178 | −0.14 | −0.05 | 1.7 | 1.7 | 0.3 | 0.89 | 0.01 |
| 333 | X. Woloszynska | 486 | −0.26 | 0.06 | 1.4 | 1.6 | 0.4 | 0.88 | 0.01 |
| 334 | HERVK22.ERVK.LTR.404.[1292 [symbol] | 83 | 0.09 | 0.23 | 0.8 | 0.3 | 1.2 | 0.88 | 0.01 |
| 335 | L1PB.L1.LINE.1520.[464] | 1561 | 0.26 | 0.47 | 2.1 | −0.4 | 0.4 | 0.88 | 0.00 |
| 336 | U2.snRNA.snRNA.1053.[44] | 53 | −0.19 | −0.12 | 1.4 | 1.4 | 0.0 | 0.87 | 0.01 |
| 337 | LTR2B.ERV1.LTR.310.[453] | 68 | 0.22 | 0.41 | 1.9 | 1.1 | 0.6 | 0.87 | 0.01 |
| 338 | L1MA5A.L1.LINE.3165.[541] | 182 | 0.14 | 0.30 | 2.2 | −0.3 | 0.6 | 0.87 | 0.00 |
| 339 | MER87.ERV1.LTR.405.[280] | 31 | 0.17 | 0.24 | 2.2 | 0.0 | 0.7 | 0.86 | 0.01 |
| 340 | AluYb.Alu.SINE.151.[123] | 2413 | 0.15 | 0.26 | 2.0 | 0.7 | 0.5 | 0.86 | 0.00 |
| 341 | body.Tip100 | 1685 | 0.04 | 0.07 | 1.4 | 1.1 | 0.8 | 0.86 | 0.00 |
| 342 | LTR60.ERV1.LTR.250.[309] | 23 | 0.25 | 0.43 | 1.8 | 0.2 | 0.5 | 0.86 | 0.00 |
| 343 | MER5A.MER1_type.DNA.35576. [symbol] | 3092 | −0.05 | −0.01 | −0.1 | 1.7 | 0.9 | 0.85 | 0.01 |
| 344 | Tigger5a.MER2_type.DNA.254 [symbol] | 159 | −0.13 | −0.05 | 1.7 | 1.5 | 0.2 | 0.85 | 0.02 |
| 345 | MER4- | 67 | 0.12 | 0.18 | 2.0 | 0.1 | 0.8 | 0.85 | 0.01 |
| 346 | LTR38B.ERV1.LTR.217.[386] | 21 | 0.14 | 0.25 | 2.1 | −0.5 | 0.8 | 0.85 | 0.01 |
| 347 | L1P5.L1.LINE.1531.[587] | 75 | −0.10 | 0.12 | 0.6 | 1.6 | 0.3 | 0.84 | 0.01 |
| 348 | L1PA15.L1.LINE.8233.[962] | 458 | 0.23 | 0.47 | 1.6 | 0.2 | 0.7 | 0.84 | 0.00 |
| 349 | MER83C.ERV1.LTR.146.[299] | 12 | 0.43 | 0.57 | 3.1 | −0.7 | 0.1 | 0.84 | 0.02 |
| 350 | MER57A- | 127 | −0.06 | 0.11 | 0.3 | 1.6 | 0.7 | 0.83 | 0.01 |
| 351 | MER67C.ERV1.LTR.1468.[341] | 103 | −0.07 | 0.06 | 1.2 | 1.0 | 0.8 | 0.83 | 0.00 |
| 352 | LTR10E.ERV1.LTR.253.[458] | 18 | 0.23 | 0.28 | 1.8 | 0.3 | 0.5 | 0.83 | 0.01 |
| 353 | MLT2C1.ERVL.LTR.2455.[246] | 148 | 0.06 | 0.15 | 3.9 | 2.9 | −0.6 | 0.83 | 0.10 |
| 354 | MER65A- | 45 | −0.27 | −0.14 | 1.3 | 1.4 | 0.1 | 0.82 | 0.01 |
| 355 | tRNA-Lys- | 6 | −0.19 | −0.39 | 0.0 | −0.2 | 1.2 | 0.82 | 0.00 |
| 356 | L1PA14.L1.LINE.3561.[941] | 187 | 0.32 | 0.58 | 2.5 | 0.2 | 0.5 | 0.82 | 0.01 |
| 357 | AluYa5.Alu.SINE.3978.[292] | 3098 | 0.18 | 0.27 | 2.5 | −0.5 | −0.1 | 0.82 | 0.01 |
| 358 | LTR16D.ERVL.LTR.1747.[217] | 64 | 0.16 | 0.26 | 1.4 | 0.7 | 0.5 | 0.82 | 0.01 |
| 359 | LTR56.ERV1.LTR.939.[275] | 83 | 0.03 | 0.11 | 2.3 | −0.3 | 1.2 | 0.82 | 0.02 |
| 360 | body.L1 | 53658 | 0.06 | 0.19 | 2.4 | −0.3 | 0.7 | 0.82 | 0.01 |
| 361 | L1PB2.L1.LINE.2666.[831] | 167 | 0.33 | 0.53 | 2.4 | −1.0 | 0.6 | 0.82 | 0.01 |
| 362 | tRNA-Ile- | 1 | −0.46 | 0.10 | −1.0 | 2.1 | 0.1 | 0.82 | 0.01 |
| 363 | LTR72.ERV1.LTR.149.[355] | 34 | 0.14 | 0.19 | 1.8 | 0.5 | 0.5 | 0.82 | 0.00 |
| 364 | LOR1b.ERV1.LTR.1086.[334] | 88 | 0.03 | 0.15 | 2.0 | −0.1 | 0.6 | 0.81 | 0.00 |
| 365 | L1PA4.L1.LINE.11462.[1602] | 723 | 0.32 | 0.66 | 1.9 | −0.3 | 0.6 | 0.81 | 0.01 |
| 366 | LTR10C- | 1 | −0.79 | −0.54 | 3.4 | 0.9 | −1.1 | 0.81 | 0.03 |
| 367 | L1P4.L1.LINE.5156.[529] | 356 | 0.20 | 0.43 | 1.7 | −0.4 | 0.7 | 0.81 | 0.00 |
| 368 | LTR43B.ERV1.LTR.213.[318] | 18 | −0.14 | 0.01 | −0.9 | 0.9 | 1.2 | 0.81 | 0.01 |
| 369 | L1M3de.L1.LINE.497.[598] | 24 | 0.24 | 0.42 | 2.0 | 0.3 | 0.7 | 0.80 | 0.01 |
| 370 | HERVL66.ERVL.LTR.104.[735] | 14 | 0.09 | 0.32 | 0.8 | 1.1 | 0.9 | 0.80 | 0.00 |
| 371 | K. Yang | 810 | −0.29 | 0.06 | 1.1 | 1.1 | 0.8 | 0.80 | 0.00 |
| 372 | Tigger3(Golem).MER2_type.D [symbol] | 306 | −0.04 | 0.08 | 0.2 | 1.7 | 0.5 | 0.80 | 0.02 |
| 373 | LTR51.ERV1.LTR.399.[424] | 34 | −0.06 | 0.04 | −0.5 | 1.7 | 0.7 | 0.80 | 0.02 |
| 374 | L1HS.L1.LINE.1696.[2016] | 365 | −0.02 | 0.32 | 1.1 | 1.1 | 0.9 | 0.80 | 0.00 |
| 375 | REP522.telo.Satellite.217. [symbol] | 96 | 0.30 | 0.14 | 1.9 | 0.8 | 0.1 | 0.80 | 0.01 |
| 376 | THE1C- | 21 | 0.48 | 0.67 | 2.0 | −0.4 | 0.8 | 0.79 | 0.01 |
| 377 | MER110- | 11 | −0.35 | −0.17 | 2.3 | 0.7 | −0.3 | 0.79 | 0.02 |
| 378 | body.tRNA | 241 | −0.02 | −0.17 | 0.0 | 0.0 | 1.5 | 0.79 | 0.01 |
| 379 | L1MD1.L1.LINE.6126.[612] | 507 | 0.03 | 0.18 | 1.6 | 0.6 | 0.7 | 0.79 | 0.00 |
| 380 | LTR66.ERVL.LTR.515.[216] | 44 | 0.08 | 0.24 | 0.6 | −0.1 | 1.3 | 0.79 | 0.00 |
| 381 | MLT1F2.MaLR.LTR.4152.[353] | 222 | 0.04 | 0.12 | 0.9 | 1.0 | 0.8 | 0.79 | 0.00 |
| 382 | L1MCa.L1.LINE.7557.[512] | 462 | 0.10 | 0.28 | 1.8 | 0.5 | 0.5 | 0.78 | 0.00 |
| 383 | MER54B.ERVL.LTR.417.[407] | 30 | 0.08 | 0.18 | 2.8 | −0.7 | 0.5 | 0.78 | 0.01 |
| 384 | MLT1A0- | 41 | 0.05 | 0.23 | 1.3 | 1.0 | 1.0 | 0.78 | 0.01 |
| 385 | LTR22B.ERVK.LTR.219.[438] | 19 | −0.06 | 0.13 | −0.1 | 1.6 | 0.6 | 0.78 | 0.07 |
| 386 | MSTC- | 20 | 0.09 | 0.26 | 0.7 | 0.4 | 1.0 | 0.78 | 0.00 |
| 387 | K. Chalitchagorn | 729 | −0.29 | 0.06 | 1.8 | 0.8 | 0.6 | 0.77 | 0.00 |
| 388 | MER20B.MER1_type.DNA.4150. [symbol] | 301 | 0.05 | 0.11 | 2.2 | −0.0 | 0.6 | 0.77 | 0.01 |
| 389 | HSATI.Satellite.Satellite. [symbol] | 3 | −0.42 | −0.25 | 1.2 | 1.3 | 0.3 | 0.77 | 0.01 |
| 390 | L1PA2.L1.LINE.4666.[2016] | 932 | −0.01 | 0.30 | 1.2 | 1.4 | 0.4 | 0.77 | 0.00 |
| 391 | L1ME1.L1.LINE.26938.[488] | 2011 | 0.03 | 0.15 | 1.5 | 0.8 | 0.5 | 0.76 | 0.00 |
| 392 | MER48.ERV1.LTR.192.[361] | 93 | 0.37 | 0.43 | 0.9 | 0.9 | 0.7 | 0.76 | 0.00 |
| 393 | L1MA4.L1.LINE.8983.[621] | 850 | 0.13 | 0.33 | 1.7 | −0.0 | 0.6 | 0.75 | 0.00 |
| 394 | MLT1C.MaLR.LTR.19102.[313] | 1596 | 0.07 | 0.12 | 1.3 | 1.2 | 0.6 | 0.75 | 0.00 |
| 395 | L1MA4A.L1.LINE.5823.[836] | 346 | 0.17 | 0.40 | 1.9 | −0.4 | 0.6 | 0.75 | 0.00 |
| 396 | MER49.ERV1.LTR.1296.[399] | 133 | 0.07 | 0.14 | 1.9 | −0.0 | 0.8 | 0.75 | 0.01 |
| 397 | MER58C.MER1_type.DNA.2066. [symbol] | 143 | −0.12 | −0.07 | 1.4 | 1.1 | −0.0 | 0.75 | 0.02 |
| 398 | LTR23.ERV1.LTR.840.[205] | 87 | −0.10 | −0.09 | 2.0 | 0.7 | −0.0 | 0.75 | 0.02 |
| 399 | L1PB3.L1.LINE.3302.[827] | 182 | 0.23 | 0.44 | 2.0 | 0.2 | 0.4 | 0.75 | 0.00 |
| 400 | AluSc.Alu.SINE.48789.[278] | 13426 | 0.03 | 0.12 | 2.0 | 0.1 | 0.4 | 0.75 | 0.00 |
| 401 | MER52A- | 4 | 0.33 | 0.62 | 1.6 | −0.5 | 0.6 | 0.74 | 0.00 |
| 402 | LTR57- | 14 | −0.07 | 0.10 | 1.0 | 0.9 | 0.9 | 0.74 | 0.01 |
| 403 | LTR55.ERV.LTR.402.[366] | 26 | −0.12 | 0.06 | −0.9 | 1.3 | 1.1 | 0.74 | 0.02 |
| 404 | LTR2C.ERV1.LTR.237.[343] | 91 | 0.03 | 0.06 | 0.1 | 1.1 | 1.0 | 0.74 | 0.01 |
| 405 | L1ME4a.L1.LINE.38237.[216] | 2335 | −0.08 | −0.02 | 1.0 | 1.4 | −0.1 | 0.74 | 0.01 |
| 406 | MER61C.ERV1.LTR.284.[360] | 23 | 0.05 | 0.28 | 1.5 | 1.7 | 0.5 | 0.73 | 0.01 |
| 407 | AluYb9.Alu.SINE.3147.[299] | 2364 | 0.16 | 0.27 | 2.0 | −0.2 | 0.5 | 0.73 | 0.00 |
| 408 | AluYg6.Alu.SINE.677.[254] | 411 | 0.10 | 0.24 | 1.6 | 0.5 | 0.6 | 0.72 | 0.00 |
| 409 | tRNA-Leu- | 2 | 0.17 | −0.23 | −0.4 | −0.2 | 1.4 | 0.71 | 0.01 |
| 410 | LTR5_Hs.ERVK.LTR.601.[880] | 134 | 0.24 | 0.34 | 1.7 | −0.8 | 1.0 | 0.71 | 0.01 |
| 411 | MER88.ERVL.LTR.110.[327] | 11 | 0.49 | 0.44 | 3.8 | −0.9 | −0.5 | 0.71 | 0.03 |
| 412 | AluSg/x.Alu.SINE.25834.[16 [symbol] | 3246 | 0.03 | 0.07 | 1.6 | 0.4 | 0.1 | 0.70 | 0.00 |
| 413 | MER5A1.MER1_type.DNA.14831 [symbol] | 917 | −0.06 | −0.02 | −0.6 | 1.4 | 1.0 | 0.70 | 0.01 |
| 414 | ERVL-E.ERVL.LTR.9525.[461] | 451 | 0.01 | 0.11 | 0.8 | 0.6 | 0.8 | 0.70 | 0.01 |
| 415 | HAL1.L1.LINE.27468.[342] | 2224 | 0.09 | 0.16 | 1.6 | 0.2 | 0.1 | 0.70 | 0.00 |
| 416 | MLT1G1- | 15 | 0.12 | 0.20 | 1.7 | 0.4 | 0.6 | 0.70 | 0.01 |
| 417 | Kanga2_a.Tc2.DNA.1465.[277 [symbol] | 109 | 0.06 | 0.15 | 1.3 | 0.5 | 0.5 | 0.69 | 0.00 |
| 418 | L1M3.L1.LINE.7700.[374] | 715 | 0.07 | 0.22 | 1.9 | −0.4 | 0.6 | 0.69 | 0.00 |
| 419 | L1MB5.L1.LINE.8813.[405] | 822 | 0.09 | 0.10 | 2.2 | −0.2 | −0.1 | 0.69 | 0.01 |
| 420 | AluYg.Alu.SINE.111.[105] | 441 | 0.09 | 0.22 | 1.7 | 0.6 | 0.1 | 0.69 | 0.00 |
| 421 | LTR16C.ERVL.LTR.9063.[251] | 462 | 0.11 | 0.18 | 1.6 | −0.2 | 0.6 | 0.69 | 0.00 |
| 422 | D20S16.Satellite.Satellite [symbol] | 18 | 1.05 | 0.93 | 2.0 | 0.8 | −0.1 | 0.69 | 0.01 |
| 423 | L1M4b.L1.LINE.7423.[511] | 454 | −0.00 | 0.16 | 1.5 | 0.5 | 0.6 | 0.69 | 0.00 |
| 424 | MER102a.MER1_type.DNA.309. [symbol] | 22 | 0.17 | 0.23 | 1.3 | 0.6 | 0.3 | 0.68 | 0.00 |
| 425 | Tigger2.MER2_type.DNA.5206 [symbol] | 578 | 0.11 | 0.21 | 1.9 | 0.3 | −0.4 | 0.68 | 0.00 |
| 426 | Charlie11.MER1_type.DNA.87 [symbol] | 5 | 0.28 | 0.37 | 1.6 | −0.0 | 0.3 | 0.68 | 0.00 |
| 427 | AluSg1.Alu.SINE.6311.[280] | 1300 | 0.02 | 0.09 | 1.8 | 0.4 | −0.1 | 0.68 | 0.00 |
| 428 | MER66A.ERV1.LTR.346.[353] | 44 | −0.04 | 0.04 | 0.0 | −0.1 | 1.1 | 0.68 | 0.00 |
| 429 | MER21A.ERV1.LTR.1793.[398] | 160 | 0.05 | 0.08 | 1.0 | 0.9 | 0.2 | 0.68 | 0.00 |
| 430 | LTR26E.ERV1.LTR.307.[375] | 43 | 0.20 | 0.14 | 1.7 | 0.2 | 0.1 | 0.68 | 0.01 |
| 431 | MARNA.Mariner.DNA.3249.[18 [symbol] | 181 | −0.10 | −0.02 | 0.0 | 1.3 | 0.1 | 0.67 | 0.01 |
| 432 | L1MCb.L1.LINE.2863.[624] | 154 | 0.04 | 0.25 | 1.7 | −0.5 | 0.6 | 0.67 | 0.00 |
| 433 | LTR39.ERV1.LTR.1193.[393] | 135 | 0.08 | 0.16 | 2.2 | 0.6 | 0.2 | 0.67 | 0.01 |
| 434 | MER61D.ERV1.LTR.263.[361] | 21 | 0.25 | 0.44 | 1.4 | −0.5 | 0.8 | 0.67 | 0.01 |
| 435 | MER58A.MER1_type.DNA.12543 [symbol] | 665 | −0.05 | 0.03 | 1.2 | 1.2 | 0.5 | 0.67 | 0.00 |
| 436 | MER101- | 39 | 0.16 | 0.25 | 1.3 | 0.6 | 0.5 | 0.67 | 0.02 |
| 437 | LTR27B.ERV1.LTR.540.[461] | 59 | 0.08 | 0.20 | 1.5 | 0.3 | 0.4 | 0.67 | 0.01 |
| 438 | AluYa.Alu.SINE.46.[110] | 3156 | 0.17 | 0.27 | 2.0 | 0.0 | −0.0 | 0.66 | 0.01 |
| 439 | HERVH.ERV1.LTR.5774.[958] | 836 | 0.95 | 1.25 | 2.0 | −0.4 | 0.3 | 0.66 | 0.01 |
| 440 | Charlie4.MER1_type.DNA.136 [symbol] | 303 | 0.03 | 0.06 | 1.9 | −0.2 | 0.2 | 0.66 | 0.00 |
| 441 | L1MC2.L1.LINE.5920.[505] | 475 | 0.02 | 0.14 | 2.0 | 0.7 | 0.4 | 0.65 | 0.01 |
| 442 | MER67B- | 1 | 0.68 | −0.72 | −0.4 | −0.7 | 1.3 | 0.65 | 0.01 |
| 443 | AluY.Alu.SINE.135761.[288] | 82937 | 0.07 | 0.18 | 1.5 | 0.0 | 0.4 | 0.65 | 0.00 |
| 444 | MER4A1- | 66 | 0.09 | 0.25 | 1.5 | 0.9 | 0.0 | 0.65 | 0.00 |
| 445 | MLT1K.MaLR.LTR.18519.[227] | 1025 | 0.02 | 0.05 | 0.0 | 0.5 | 0.9 | 0.65 | 0.00 |
| 446 | Arthur1.Tip100.DNA.4836.[2 [symbol] | 269 | −0.01 | 0.07 | 0.7 | 0.6 | 0.9 | 0.64 | 0.01 |
| 447 | MER52A.ERV1.LTR.1824.[1041 [symbol] | 206 | 0.07 | 0.13 | 1.0 | 0.5 | 0.6 | 0.64 | 0.00 |
| 448 | BLACKJACK.AcHobo.DNA.2099. [symbol] | 104 | 0.04 | 0.16 | 1.5 | 0.4 | 0.4 | 0.64 | 0.00 |
| 449 | MER4A1_.ERV1.LTR.380.[486] | 103 | 0.07 | 0.22 | 1.6 | 0.1 | 0.5 | 0.64 | 0.01 |
| 450 | LTR45C.ERV1.LTR.393.[324] | 34 | 0.29 | 0.35 | 2.7 | −1.0 | −0.2 | 0.64 | 0.01 |
| 451 | LTR53.ERVL.LTR.666.[362] | 41 | 0.12 | 0.21 | 1.3 | 0.1 | 0.5 | 0.63 | 0.00 |
| 452 | LTR12D.ERV1.LTR.502.[810] | 308 | 0.30 | 0.40 | 1.7 | 1.0 | 0.0 | 0.63 | 0.01 |
| 453 | MER31A- | 11 | 0.17 | 0.33 | 1.9 | −0.4 | −0.1 | 0.63 | 0.01 |
| 454 | MER44C.MER2_type.DNA.802.[ [symbol] | 57 | −0.02 | 0.07 | 0.5 | 1.4 | 0.3 | 0.63 | 0.00 |
| 455 | LTR48B- | 3 | −0.52 | −0.35 | 2.3 | 0.3 | −0.2 | 0.62 | 0.02 |
| 456 | L1MC.L1.LINE.16426.[272] | 9792 | −0.00 | 0.09 | 1.2 | 0.6 | 0.7 | 0.62 | 0.00 |
| 457 | Ricksha.MuDR.DNA.1551.[287 [symbol] | 86 | 0.03 | 0.12 | 2.1 | 0.1 | −0.0 | 0.62 | 0.00 |
| 458 | MER57B.ERV1.LTR.1225.[343] | 94 | 0.04 | 0.24 | 1.0 | 1.1 | 0.3 | 0.62 | 0.00 |
| 459 | MER57B- | 35 | 0.03 | 0.27 | 1.3 | 0.7 | 0.4 | 0.62 | 0.00 |
| 460 | MLT1H2.MaLR.LTR.4227.[216] | 208 | 0.11 | 0.15 | 1.6 | −0.4 | −0.0 | 0.61 | 0.00 |
| 461 | LTR38- | 5 | −0.19 | −0.26 | 0.4 | −0.0 | 0.9 | 0.61 | 0.00 |
| 462 | MER61B.ERV1.LTR.409.[332] | 43 | 0.16 | 0.24 | 1.7 | −0.2 | −0.1 | 0.61 | 0.00 |
| 463 | L1PA15- | 64 | −0.06 | 0.21 | 0.5 | 0.7 | 0.6 | 0.61 | 0.00 |
| 464 | MER50- | 36 | 0.10 | 0.20 | 0.0 | 0.6 | 0.8 | 0.61 | 0.01 |
| 465 | LSAU.Satellite.Satellite.4 [symbol] | 10 | 0.34 | −0.08 | −0.7 | 1.8 | 0.4 | 0.60 | 0.01 |
| 466 | Charlie10.MER1_type.DNA.22 [symbol] | 133 | −0.03 | 0.06 | 0.5 | 0.6 | 0.8 | 0.60 | 0.01 |
| 467 | L1P4d.L1.LINE.204.[1102] | 17 | 0.21 | 0.39 | 1.1 | 0.5 | 0.3 | 0.60 | 0.00 |
| 468 | LTR12.ERV1.LTR.964.[633] | 3627 | 0.33 | 0.40 | 1.3 | 0.7 | 0.5 | 0.60 | 0.00 |
| 469 | L1P3.L1.LINE.4518.[756] | 219 | 0.17 | 0.39 | 1.6 | −0.3 | −0.1 | 0.60 | 0.00 |
| 470 | L1MEa.L1.LINE.1665.[407] | 115 | 0.03 | 0.16 | 1.0 | 0.8 | 0.7 | 0.60 | 0.00 |
| 471 | MER46B.MER2_type.DNA.2000. [symbol] | 134 | −0.13 | −0.05 | 0.2 | 1.1 | 0.4 | 0.59 | 0.01 |
| 472 | MER7A.MER2_type.DNA.5173.[ [symbol] | 322 | −0.08 | −0.00 | −0.1 | 1.3 | −0.2 | 0.59 | 0.01 |
| 473 | tRNA-Val- | 7 | 0.29 | 0.16 | 1.0 | 0.7 | 0.1 | 0.59 | 0.01 |
| 474 | MER4A- | 10 | 0.18 | 0.18 | 1.3 | 0.1 | 0.2 | 0.59 | 0.00 |
| 475 | L1PB1.L1.LINE.11421.[1064] | 653 | 0.28 | 0.51 | 1.6 | 0.4 | −0.2 | 0.58 | 0.00 |
| 476 | Charlie4a.MER1_type.DNA.23 [symbol] | 212 | 0.04 | 0.07 | 2.0 | −0.5 | 0.3 | 0.58 | 0.00 |
| 477 | Tigger6.MER2_type.DNA.253. [symbol] | 77 | 0.10 | 0.18 | 1.1 | 0.0 | 0.5 | 0.58 | 0.00 |
| 478 | LTR71A.ERV1.LTR.164.[385] | 24 | 0.25 | 0.41 | 1.8 | 0.1 | 0.4 | 0.58 | 0.01 |
| 479 | MER89- | 25 | 0.16 | 0.21 | 1.9 | −0.7 | 0.2 | 0.57 | 0.01 |
| 480 | MLT1E1.MaLR.LTR.2001.[322] | 176 | 0.03 | 0.14 | 1.0 | 0.7 | 0.3 | 0.57 | 0.00 |
| 481 | MER58.MER1_type.DNA.2330.[ [symbol] | 1464 | −0.08 | −0.02 | −0.8 | 1.5 | 0.3 | 0.57 | 0.01 |
| 482 | L1MB2.L1.LINE.7699.[402] | 596 | 0.04 | 0.13 | 1.2 | 0.4 | 0.3 | 0.57 | 0.00 |
| 483 | MER75B.PiggyBac.DNA.111.[1 [symbol] | 4 | 0.17 | 0.31 | 1.4 | −0.7 | 0.9 | 0.56 | 0.01 |
| 484 | L1ME3A.L1.LINE.16330.[362] | 1127 | 0.00 | 0.09 | 1.0 | 0.7 | 0.4 | 0.56 | 0.00 |
| 485 | MER119.MER1_type.DNA.1169. [symbol] | 93 | −0.14 | −0.06 | −0.2 | 1.0 | 0.5 | 0.56 | 0.01 |
| 486 | MER106A.MER1_type.DNA.689. [symbol] | 45 | 0.04 | 0.06 | 0.4 | 0.9 | 0.8 | 0.56 | 0.00 |
| 487 | HERVL40.ERVL.LTR.1217.[378 [symbol] | 49 | −0.01 | 0.12 | 0.2 | 1.8 | 0.3 | 0.56 | 0.02 |
| 488 | L1M2.L1.LINE.9352.[648] | 523 | 0.13 | 0.32 | 1.2 | −0.0 | 0.3 | 0.56 | 0.00 |
| 489 | MER41- | 29 | −0.06 | 0.14 | −0.4 | 1.0 | 0.0 | 0.56 | 0.00 |
| 490 | body.Mariner | 855 | −0.05 | 0.08 | −0.7 | 1.5 | 0.4 | 0.56 | 0.01 |
| 491 | Zaphod.Tip100.DNA.2063.[32 [symbol] | 166 | 0.00 | 0.10 | 1.6 | 0.4 | 0.3 | 0.55 | 0.00 |
| 492 | MER34B.ERV1.LTR.1289.[331] | 116 | −0.08 | 0.03 | −0.3 | 1.4 | 0.4 | 0.54 | 0.01 |
| 493 | tRNA-Glu- | 13 | −0.27 | −0.34 | 1.3 | −0.2 | 0.5 | 0.54 | 0.00 |
| 494 | LTR12C.ERV1.LTR.2616.[1340 [symbol] | 2692 | 0.31 | 0.36 | 1.4 | 0.5 | −0.3 | 0.54 | 0.01 |
| 495 | AluJ/FRAM.Alu.SINE.1750.[7 [symbol] | 116 | 0.01 | 0.09 | 0.6 | 0.3 | 0.9 | 0.54 | 0.00 |
| 496 | L1M3a.L1.LINE.683.[590] | 21 | 0.07 | 0.20 | 1.5 | 0.0 | 0.3 | 0.54 | 0.00 |
| 497 | HERVL-C1.ERVL.LTR.7.[980] | 1 | 0.09 | 0.79 | 0.6 | 0.9 | 0.5 | 0.54 | 0.01 |
| 498 | L1M.L1.LINE.11466.[335] | 43253 | 0.02 | 0.13 | 1.5 | 0.4 | 0.1 | 0.54 | 0.00 |
| 499 | MER72.ERV1.LTR.736.[435] | 65 | 0.08 | 0.20 | 1.0 | 0.5 | 0.4 | 0.54 | 0.00 |
| 500 | L1M1.L1.LINE.10118.[932] | 594 | 0.10 | 0.33 | 0.9 | 0.6 | 0.5 | 0.54 | 0.00 |
| 501 | L1MD.L1.LINE.10847.[339] | 3480 | −0.01 | 0.11 | 0.8 | 0.6 | 0.6 | 0.54 | 0.00 |
| 502 | body.SINE | 197952 | 0.02 | 0.09 | 1.1 | 0.3 | 0.3 | 0.53 | 0.00 |
| 503 | body.Tc2 | 428 | −0.01 | 0.05 | 0.6 | 0.4 | 0.5 | 0.53 | 0.00 |
| 504 | L1MB8.L1.LINE.15156.[428] | 1204 | 0.04 | 0.12 | 1.5 | −0.2 | 0.2 | 0.53 | 0.00 |
| 505 | LTR16B.ERVL.LTR.3456.[292] | 190 | 0.15 | 0.26 | 2.0 | −0.3 | 0.1 | 0.53 | 0.00 |
| 506 | MIR.MIR.SINE.203898.[147] | 35264 | −0.03 | −0.03 | 0.0 | 1.4 | 0.5 | 0.52 | 0.01 |
| 507 | L1MEc.L1.LINE.21328.[480] | 1317 | −0.03 | 0.12 | 1.3 | −0.1 | 0.5 | 0.52 | 0.00 |
| 508 | MER90a.ERV1.LTR.1165.[326] | 91 | 0.17 | 0.17 | 1.8 | 0.3 | −0.5 | 0.52 | 0.01 |
| 509 | L1P4c.L1.LINE.93.[937] | 7 | 0.28 | 0.47 | 1.3 | −0.6 | 0.7 | 0.52 | 0.00 |
| 510 | LTR12_.ERV1.LTR.664.[497] | 3364 | 0.32 | 0.39 | 0.9 | −0.3 | 0.5 | 0.51 | 0.00 |
| 511 | un.genebody | 54202 | −0.06 | −0.12 | 0.5 | −0.1 | 0.7 | 0.51 | 0.00 |
| 512 | L1MA3.L1.LINE.8131.[987] | 542 | 0.17 | 0.39 | 1.5 | −0.4 | 0.4 | 0.51 | 0.00 |
| 513 | MER34D- | 1 | −0.56 | −0.44 | 2.0 | 0.1 | −0.3 | 0.51 | 0.01 |
| 514 | L1MC4.L1.LINE.29881.[332] | 4239 | −0.02 | 0.05 | 0.9 | 0.5 | 0.3 | 0.50 | 0.00 |
| 515 | body.acro | 12 | 0.37 | 0.80 | 1.2 | 0.4 | 0.5 | 0.50 | 0.00 |
| 516 | L1MCc.L1.LINE.6889.[452] | 307 | −0.03 | 0.15 | 1.1 | 0.9 | −0.2 | 0.50 | 0.00 |
| 517 | L1M3e.L1.LINE.981.[813] | 34 | 0.15 | 0.33 | 1.3 | −0.3 | 0.1 | 0.50 | 0.00 |
| 518 | MER81.AcHobo.DNA.3623.[94] | 196 | 0.05 | 0.08 | 0.7 | 0.0 | 0.6 | 0.50 | 0.00 |
| 519 | LTR37A.ERV1.LTR.1957.[224] | 109 | 0.01 | 0.13 | 1.1 | 1.0 | −0.1 | 0.50 | 0.00 |
| 520 | MER34C_- | 1 | 0.20 | −0.09 | −0.1 | 1.3 | 0.3 | 0.50 | 0.04 |
| 521 | L1P4a.L1.LINE.731.[1306] | 55 | 0.28 | 0.55 | 1.5 | 0.2 | −0.1 | 0.49 | 0.00 |
| 522 | MER31- | 65 | −0.12 | 0.02 | 0.4 | 0.8 | −0.1 | 0.49 | 0.00 |
| 523 | 7SK.RNA.RNA.715.[165] | 58 | 0.21 | 0.32 | 1.1 | −0.5 | 0.4 | 0.49 | 0.00 |
| 524 | LTR40b.ERVL.LTR.1229.[270] | 62 | 0.05 | 0.15 | 0.5 | 0.5 | 0.5 | 0.49 | 0.00 |
| 525 | SATR2.Satellite.Satellite. [symbol] | 60 | −0.19 | −0.12 | 2.3 | −0.1 | −0.6 | 0.49 | 0.02 |
| 526 | L1MD3.L1.LINE.7482.[311] | 573 | −0.06 | 0.02 | −0.5 | 1.0 | 0.6 | 0.49 | 0.00 |
| 527 | Tigger8.MER2_type.DNA.837. [symbol] | 50 | 0.09 | 0.12 | 1.7 | −0.3 | 0.2 | 0.48 | 0.00 |
| 528 | LTR15.ERV1.LTR.311.[376] | 25 | 0.04 | 0.18 | 1.0 | −0.1 | 0.6 | 0.48 | 0.00 |
| 529 | AluYh9.Alu.SINE.183.[159] | 56 | 0.13 | 0.21 | 1.2 | 0.7 | 0.0 | 0.48 | 0.00 |
| 530 | MIRb.MIR.SINE.278083.[144] | 17373 | −0.03 | −0.03 | −0.3 | 1.5 | 0.1 | 0.48 | 0.01 |
| 531 | L1M4.L1.LINE.19504.[380] | 2246 | 0.00 | 0.13 | 1.1 | 0.0 | 0.2 | 0.48 | 0.00 |
| 532 | LTR24.ERV1.LTR.308.[302] | 101 | −0.09 | −0.03 | 0.5 | 0.8 | 0.3 | 0.47 | 0.00 |
| 533 | L1MEb.L1.LINE.5722.[435] | 329 | −0.05 | 0.09 | 0.9 | 0.5 | 0.3 | 0.47 | 0.00 |
| 534 | MER41D.ERV1.LTR.435.[299] | 62 | −0.15 | −0.10 | −0.1 | −0.3 | 0.8 | 0.47 | 0.00 |
| 535 | L1M5.L1.LINE.63689.[284] | 4051 | −0.02 | 0.07 | 1.0 | 0.4 | 0.4 | 0.47 | 0.00 |
| 536 | MER45A.Tip100.DNA.3214.[14 [symbol] | 243 | 0.09 | 0.08 | 0.9 | 0.4 | 0.2 | 0.47 | 0.00 |
| 537 | Alu.Alu.SINE.6244.[52] | 181521 | 0.03 | 0.10 | 1.1 | 0.6 | −0.1 | 0.47 | 0.00 |
| 538 | MER3.MER1_type.DNA.10996.[ [symbol] | 2609 | −0.05 | 0.01 | 0.5 | 0.8 | 0.5 | 0.47 | 0.00 |
| 539 | ACRO1.acro.Satellite.40.[7 [symbol] | 12 | 0.37 | 0.80 | 1.0 | 1.0 | 0.1 | 0.46 | 0.00 |
| 540 | ERVL-C1.ERVL.LTR.334.[700] | 18 | −0.16 | 0.09 | −0.4 | 1.4 | 0.4 | 0.46 | 0.01 |
| 541 | LTR54.ERV1.LTR.1087.[261] | 128 | −0.07 | 0.02 | −0.1 | 0.4 | 0.6 | 0.46 | 0.01 |
| 542 | AluYd.Alu.SINE.65.[107] | 576 | 0.06 | 0.15 | 0.9 | 0.4 | 0.5 | 0.46 | 0.00 |
| 543 | HERVS71.ERV1.LTR.149.[1952 [symbol] | 83 | −0.44 | −0.45 | 1.7 | −1.0 | 0.7 | 0.45 | 0.01 |
| 544 | MER101.ERV1.LTR.379.[292] | 84 | 0.04 | 0.12 | 0.8 | 0.1 | 0.4 | 0.45 | 0.00 |
| 545 | LTR40c.ERVL.LTR.609.[321] | 24 | −0.14 | −0.01 | −0.3 | 1.2 | 0.3 | 0.45 | 0.00 |
| 546 | HY4.scRNA.scRNA.157.[79] | 10 | 0.03 | 0.01 | −0.7 | 0.9 | 0.4 | 0.45 | 0.01 |
| 547 | HSMAR2.Mariner.DNA.1721.[6 [symbol] | 108 | −0.10 | 0.05 | −0.5 | 0.8 | 0.7 | 0.45 | 0.00 |
| 548 | Charliela.MER1_type.DNA.36 [symbol] | 337 | −0.16 | −0.07 | 1.0 | 0.6 | −0.2 | 0.44 | 0.01 |
| 549 | MER75.PiggyBac.DNA.551.[33 [symbol] | 44 | 0.07 | 0.15 | 1.7 | 0.1 | 0.2 | 0.44 | 0.01 |
| 550 | AluYd8.Alu.SINE.853.[274] | 562 | 0.06 | 0.14 | 1.4 | 0.0 | 0.3 | 0.44 | 0.00 |
| 551 | L1MD2.L1.LINE.12388.[485] | 944 | 0.03 | 0.13 | 1.0 | 0.5 | −0.4 | 0.44 | 0.00 |
| 552 | MER76- | 9 | 0.03 | 0.20 | 0.5 | 0.0 | 0.5 | 0.44 | 0.00 |
| 553 | L1M2a.L1.LINE.515.[883] | 26 | −0.09 | 0.19 | 0.6 | 0.8 | 0.4 | 0.44 | 0.00 |
| 554 | L1MA10.L1.LINE.5419.[291] | 275 | −0.00 | 0.12 | 1.3 | 0.3 | 0.1 | 0.43 | 0.00 |
| 555 | Tigger6a.MER2_type.DNA.681 [symbol] | 54 | 0.09 | 0.18 | 1.3 | −0.5 | 0.1 | 0.43 | 0.00 |
| 556 | Tigger7.MER2_type.DNA.4405 [symbol] | 196 | −0.05 | 0.05 | 0.7 | 0.6 | 0.2 | 0.43 | 0.00 |
| 557 | tRNA-Thr- | 4 | −0.41 | −0.23 | 0.7 | 0.5 | 0.3 | 0.43 | 0.01 |
| 558 | L1ME2.L1.LINE.15797.[423] | 1122 | 0.01 | 0.11 | 1.1 | 0.0 | −0.3 | 0.43 | 0.00 |
| 559 | L1PBa.L1.LINE.1915.[1205] | 171 | 0.29 | 0.57 | 1.2 | −0.3 | 0.3 | 0.43 | 0.00 |
| 560 | Charlie1.MER1_type.DNA.491 [symbol] | 1048 | −0.12 | −0.04 | 0.8 | 0.8 | −0.2 | 0.43 | 0.01 |
| 561 | L1M4c.L1.LINE.7787.[595] | 456 | 0.02 | 0.19 | 0.7 | 0.3 | 0.3 | 0.43 | 0.00 |
| 562 | MER107.MER1_type.DNA.320.[ [symbol] | 13 | −0.10 | −0.24 | −0.0 | 0.0 | 0.8 | 0.42 | 0.00 |
| 563 | FAM.Alu.SINE.4424.[149] | 355 | 0.01 | 0.06 | 1.3 | 0.4 | 0.0 | 0.42 | 0.00 |
| 564 | MLT1E1A.MaLR.LTR.1054.[326 [symbol] | 63 | −0.03 | 0.08 | −0.1 | 1.0 | 0.0 | 0.42 | 0.00 |
| 565 | LTR1B- | 2 | 0.20 | 0.20 | 0.6 | −0.7 | 0.8 | 0.42 | 0.00 |
| 566 | L1M3c.L1.LINE.1069.[583] | 43 | −0.07 | 0.13 | 0.5 | 0.7 | 0.3 | 0.42 | 0.00 |
| 567 | MER65C.ERV1.LTR.683.[311] | 67 | −0.04 | 0.03 | −0.4 | 1.0 | 0.1 | 0.42 | 0.00 |
| 568 | MER34- | 28 | −0.04 | 0.09 | 0.5 | 0.5 | 0.4 | 0.41 | 0.00 |
| 569 | LTR3B.ERVK.LTR.90.[391] | 38 | 0.11 | 0.18 | 0.8 | 0.7 | 0.1 | 0.41 | 0.00 |
| 570 | tRNA-SeC(e)- | 1 | −0.24 | −0.31 | 0.5 | 0.2 | 0.6 | 0.41 | 0.01 |
| 571 | L1M2c.L1.LINE.585.[856] | 23 | 0.26 | 0.45 | 1.5 | 0.0 | 0.1 | 0.40 | 0.00 |
| 572 | MER6B.MER2_type.DNA.656.[1 [symbol] | 26 | −0.11 | −0.03 | 1.1 | −0.2 | 0.5 | 0.40 | 0.01 |
| 573 | L1MC4a.L1.LINE.28404.[336] | 2100 | −0.02 | 0.05 | 0.4 | 0.4 | 0.4 | 0.40 | 0.00 |
| 574 | AluJ.Alu.SINE.2554.[134] | 21348 | −0.00 | 0.02 | 1.0 | 0.0 | 0.0 | 0.40 | 0.00 |
| 575 | MER65C- | 9 | 0.24 | 0.43 | 0.7 | 0.5 | 0.2 | 0.40 | 0.00 |
| 576 | MER53.DNA.DNA.5686.[128] | 332 | −0.05 | −0.05 | 2.2 | −0.8 | 0.2 | 0.39 | 0.02 |
| 577 | LTR10G.ERV1.LTR.116.[432] | 18 | 0.30 | 0.43 | 1.6 | −0.4 | −0.1 | 0.39 | 0.00 |
| 578 | AluYa8.Alu.SINE.107.[226] | 52 | 0.03 | 0.11 | 0.8 | 0.0 | 0.3 | 0.39 | 0.00 |
| 579 | body.CR1 | 2938 | −0.03 | 0.02 | −0.2 | 0.7 | 0.2 | 0.39 | 0.00 |
| 580 | tRNA-Phe- | 7 | 0.30 | 0.38 | 1.7 | −0.6 | 0.0 | 0.38 | 0.00 |
| 581 | GSAT.centr.Satellite.65.[3 [symbol] | 144 | −0.20 | −0.25 | 2.0 | −0.5 | 0.1 | 0.38 | 0.01 |
| 582 | L1MEd.L1.LINE.15988.[333] | 903 | −0.04 | 0.08 | 0.8 | 0.5 | 0.3 | 0.38 | 0.00 |
| 583 | MER1B.MER1_type.DNA.5233.[ [symbol] | 632 | 0.06 | 0.09 | 1.0 | 0.3 | 0.0 | 0.38 | 0.01 |
| 584 | L1MEe.L1.LINE.12492.[333] | 750 | −0.01 | 0.12 | 0.9 | 0.5 | 0.1 | 0.38 | 0.00 |
| 585 | L1ME3.L1.LINE.7572.[348] | 3426 | 0.00 | 0.09 | 0.9 | 0.4 | 0.1 | 0.37 | 0.00 |
| 586 | body.MER2_type | 5338 | −0.01 | 0.09 | 0.1 | 0.3 | 0.0 | 0.37 | 0.00 |
| 587 | MER44B.MER2_type.DNA.1256. [symbol] | 106 | −0.02 | 0.09 | 0.6 | 0.4 | 0.3 | 0.37 | 0.00 |
| 588 | AluJb.Alu.SINE.125688.[259 [symbol] | 10131 | −0.00 | 0.03 | 0.9 | 0.0 | 0.4 | 0.37 | 0.00 |
| 589 | L1MC5.L1.LINE.20412.[260] | 1523 | −0.03 | 0.03 | 0.5 | −0.1 | 0.4 | 0.37 | 0.00 |
| 590 | L1MB4.L1.LINE.8956.[415] | 710 | −0.01 | 0.08 | 0.7 | 0.1 | 0.4 | 0.36 | 0.00 |
| 591 | MER46C.MER2_type.DNA.2705. [symbol] | 186 | −0.08 | −0.04 | 0.7 | 0.5 | −0.4 | 0.36 | 0.00 |
| 592 | body.PiggyBac | 121 | −0.04 | 0.06 | −0.5 | 0.3 | 0.6 | 0.36 | 0.00 |
| 593 | ERVL- | 17 | 0.19 | 0.29 | 2.0 | −0.6 | 0.2 | 0.36 | 0.01 |
| 594 | CER.Satellite.Satellite.35 [symbol] | 4 | 0.94 | 1.23 | 1.4 | 0.1 | −0.0 | 0.36 | 0.01 |
| 595 | LTR59.ERV1.LTR.121.[337] | 11 | 0.05 | 0.16 | 1.0 | 0.5 | 0.2 | 0.35 | 0.00 |
| 596 | Merlin1_HS.Merlin.DNA.53.[ [symbol] | 1 | −0.38 | −0.64 | 0.5 | 0.2 | 0.5 | 0.35 | 0.00 |
| 597 | L1ME3B.L1.LINE.27528.[351] | 1764 | 0.01 | 0.11 | 0.6 | 0.0 | 0.3 | 0.35 | 0.00 |
| 598 | MER105.DNA.DNA.712.[145] | 37 | −0.15 | −0.02 | −0.1 | 0.6 | 0.3 | 0.35 | 0.00 |
| 599 | MER90.ERV1.LTR.678.[317] | 148 | 0.16 | 0.16 | 1.5 | −1.1 | 0.2 | 0.35 | 0.01 |
| 600 | MER61- | 11 | 0.04 | 0.28 | 0.7 | 0.0 | 0.5 | 0.34 | 0.00 |
| 601 | HY3.scRNA.scRNA.509.[87] | 51 | −0.01 | 0.04 | 0.5 | 0.5 | 0.3 | 0.34 | 0.00 |
| 602 | MER4D0.ERV1.LTR.463.[369] | 32 | 0.17 | 0.23 | 1.5 | −0.1 | −0.3 | 0.34 | 0.00 |
| 603 | MLT1D- | 38 | 0.07 | 0.17 | 1.3 | −0.2 | −0.1 | 0.34 | 0.00 |
| 604 | MER41G.ERV1.LTR.105.[415] | 17 | 0.20 | 0.31 | 1.6 | −0.4 | −0.2 | 0.34 | 0.00 |
| 605 | body.telo | 134 | 0.21 | 0.11 | 0.8 | −0.2 | 0.3 | 0.33 | 0.00 |
| 606 | MER65B.ERV1.LTR.238.[194] | 22 | −0.09 | −0.00 | −1.0 | 0.5 | 0.6 | 0.33 | 0.00 |
| 607 | LTR44.ERV1.LTR.201.[309] | 23 | 0.14 | 0.18 | 0.7 | 0.0 | 0.2 | 0.33 | 0.00 |
| 608 | tRNA-Ala- | 10 | −0.13 | −0.32 | −0.4 | 0.7 | 0.4 | 0.33 | 0.00 |
| 609 | tRNA-Asn- | 7 | −0.10 | −0.46 | 1.0 | 0.2 | 0.1 | 0.33 | 0.00 |
| 610 | MER65D.ERV1.LTR.415.[277] | 42 | −0.13 | −0.03 | 0.1 | 0.6 | 0.3 | 0.33 | 0.01 |
| 611 | MER41A- | 46 | 0.01 | 0.17 | 0.7 | 0.6 | 0.3 | 0.33 | 0.00 |
| 612 | U5.snRNA.snRNA.205.[55] | 7 | −0.26 | −0.18 | 0.5 | −0.1 | 0.2 | 0.33 | 0.01 |
| 613 | tRNA-Val- | 10 | −0.25 | −0.41 | 0.9 | −0.0 | 0.5 | 0.32 | 0.00 |
| 614 | MER33.MER1_type.DNA.8040.[ [symbol] | 581 | −0.05 | −0.02 | 0.5 | 0.7 | −0.3 | 0.32 | 0.00 |
| 615 | MER66B- | 9 | 0.11 | 0.17 | 1.2 | −0.4 | 0.1 | 0.32 | 0.00 |
| 616 | LTR76- | 4 | −0.10 | 0.40 | −0.7 | 0.5 | 0.6 | 0.32 | 0.01 |
| 617 | LTR37B.ERV1.LTR.1958.[226] | 99 | −0.09 | 0.01 | 0.1 | 0.5 | 0.2 | 0.32 | 0.00 |
| 618 | ORSL.Tip100.DNA.1181.[165] | 101 | 0.04 | 0.07 | 1.1 | −0.4 | 0.3 | 0.32 | 0.00 |
| 619 | L1MDa.L1.LINE.9760.[522] | 651 | −0.03 | 0.12 | 1.2 | −0.0 | −0.2 | 0.32 | 0.00 |
| 620 | L1P4e.L1.LINE.221.[996] | 8 | 0.62 | 0.94 | 0.8 | 0.6 | −0.0 | 0.32 | 0.00 |
| 621 | MER66B.ERV1.LTR.1407.[302] | 138 | 0.09 | 0.12 | 0.8 | 0.2 | 0.4 | 0.31 | 0.00 |
| 622 | PRIMAX- | 10 | −0.12 | −0.05 | −0.4 | 0.9 | −0.1 | 0.31 | 0.00 |
| 623 | LTR45B.ERV1.LTR.417.[324] | 34 | −0.17 | −0.12 | 0.9 | 0.3 | −0.1 | 0.30 | 0.01 |
| 624 | MER6C.MER2_type.DNA.136.[1 [symbol] | 3 | −0.30 | −0.13 | −0.5 | 0.7 | 0.4 | 0.30 | 0.00 |
| 625 | LOR1b- | 23 | −0.01 | 0.10 | 0.4 | 0.3 | 0.3 | 0.30 | 0.00 |
| 626 | tRNA-Ser- | 6 | −0.32 | −0.63 | 0.9 | −0.3 | 0.6 | 0.30 | 0.00 |
| 627 | MER51- | 5 | −0.23 | −0.00 | 0.9 | 0.4 | 0.1 | 0.29 | 0.00 |
| 628 | MER4A1_- | 6 | 0.16 | 0.37 | 1.4 | 0.7 | −0.5 | 0.29 | 0.00 |
| 629 | AluSg.Alu.SINE.80825.[289] | 24024 | −0.00 | 0.06 | 1.0 | 0.0 | −0.2 | 0.29 | 0.00 |
| 630 | Kanga1.Tc2.DNA.3485.[171] | 219 | −0.06 | 0.00 | 0.4 | 0.4 | 0.0 | 0.29 | 0.00 |
| 631 | AluYc5.Alu.SINE.496.[51] | 56 | 0.07 | 0.11 | 1.3 | −0.7 | 0.0 | 0.28 | 0.00 |
| 632 | AluS.Alu.SINE.2733.[76] | 102736 | −0.00 | 0.05 | 0.3 | 0.0 | 0.4 | 0.28 | 0.00 |
| 633 | Ricksha_c.MuDR.DNA.121.[85 [symbol] | 8 | −0.15 | −0.00 | −0.7 | 1.3 | 0.1 | 0.28 | 0.01 |
| 634 | LTR64.ERV1.LTR.352.[292] | 53 | −0.04 | 0.02 | 0.1 | 0.2 | 0.6 | 0.28 | 0.01 |
| 635 | MER21B- | 12 | −0.14 | 0.02 | 0.4 | 0.5 | 0.1 | 0.27 | 0.00 |
| 636 | LTR57.ERVL.LTR.234.[327] | 29 | 0.01 | 0.12 | 1.0 | −0.2 | 0.2 | 0.27 | 0.00 |
| 637 | MER93B.ERV1.LTR.989.[269] | 89 | −0.05 | −0.00 | −0.3 | 0.7 | 0.0 | 0.27 | 0.00 |
| 638 | HSAT4.centr.Satellite.96.[ [symbol] | 7 | 0.36 | 0.65 | 1.2 | −0.4 | 0.1 | 0.27 | 0.01 |
| 639 | MER72- | 5 | −0.33 | −0.10 | −0.1 | 0.9 | −0.2 | 0.26 | 0.01 |
| 640 | MER103.DNA.DNA.6812.[111] | 292 | −0.01 | 0.06 | −0.4 | −0.3 | 0.5 | 0.26 | 0.00 |
| 641 | LTR56- | 8 | 0.01 | 0.28 | 0.9 | −0.4 | 0.2 | 0.26 | 0.00 |
| 642 | Tigger4(Zombi).MER2_type.D [symbol] | 103 | −0.05 | 0.10 | 0.9 | −0.5 | 0.2 | 0.26 | 0.00 |
| 643 | AluYc.Alu.SINE.50.[73] | 241 | 0.05 | 0.08 | 1.6 | −0.7 | −0.1 | 0.26 | 0.00 |
| 644 | MER61F- | 5 | −0.01 | 0.23 | 0.7 | 0.6 | −0.0 | 0.25 | 0.00 |
| 645 | MER52C.ERV1.LTR.366.[827] | 36 | 0.10 | 0.09 | 1.9 | 0.1 | −0.4 | 0.25 | 0.01 |
| 646 | MER2.MER2_type.DNA.8174.[2 [symbol] | 7533 | 0.00 | 0.08 | 1.1 | −0.4 | −0.1 | 0.25 | 0.00 |
| 647 | tRNA-Arg- | 3 | −0.43 | −0.53 | 0.7 | 0.5 | −0.2 | 0.25 | 0.00 |
| 648 | MER82.MER2_type.DNA.3129.[ [symbol] | 255 | −0.12 | −0.03 | −0.7 | 0.7 | 0.1 | 0.25 | 0.00 |
| 649 | MER70A- | 4 | −0.18 | 0.08 | 0.6 | 1.1 | −0.2 | 0.25 | 0.00 |
| 650 | PRIMA4_LTR- | 8 | 0.29 | 0.42 | 1.1 | −0.2 | 0.5 | 0.25 | 0.00 |
| 651 | AluJo.Alu.SINE.138391.[257 [symbol] | 11068 | −0.00 | 0.02 | 0.5 | 0.2 | 0.2 | 0.25 | 0.00 |
| 652 | tRNA-Pro- | 3 | −0.05 | −0.24 | 0.1 | 0.6 | 0.2 | 0.24 | 0.00 |
| 653 | MER65- | 30 | −0.06 | 0.05 | −0.1 | 0.5 | 0.2 | 0.24 | 0.00 |
| 654 | LTR30.ERV1.LTR.170.[560] | 43 | 0.34 | 0.58 | 0.7 | −0.2 | 0.1 | 0.24 | 0.00 |
| 655 | MER57- | 15 | −0.07 | 0.07 | 0.0 | −0.0 | 0.5 | 0.24 | 0.00 |
| 656 | MLT1- | 15 | −0.03 | 0.10 | 0.6 | −0.4 | 0.4 | 0.24 | 0.00 |
| 657 | MLT1L.MaLR.LTR.11354.[209] | 527 | −0.00 | 0.06 | 0.0 | 0.4 | 0.3 | 0.24 | 0.00 |
| 658 | MER51E- | 1 | −0.82 | −0.42 | 1.0 | 0.3 | −0.5 | 0.24 | 0.01 |
| 659 | LTR47A.ERVL.LTR.873.[260] | 59 | −0.09 | 0.03 | −0.4 | −0.1 | 0.5 | 0.24 | 0.01 |
| 660 | LTR24C- | 1 | −0.24 | −0.12 | −0.7 | 0.5 | 0.3 | 0.24 | 0.00 |
| 661 | AluYa5/8.Alu.SINE.112.[108 [symbol] | 33 | 0.21 | 0.31 | 1.6 | −0.7 | 0.3 | 0.23 | 0.00 |
| 662 | MER91B.Tip100.DNA.1429.[10 [symbol] | 66 | −0.17 | −0.17 | 2.3 | −1.1 | −0.0 | 0.23 | 0.01 |
| 663 | LTR50.ERVL.LTR.2809.[267] | 143 | 0.03 | 0.10 | −0.5 | −0.8 | 0.6 | 0.23 | 0.00 |
| 664 | HERV30.ERV1.LTR.61.[1647] | 13 | 0.23 | 0.39 | 1.0 | −0.6 | 0.1 | 0.23 | 0.00 |
| 665 | Charlie6.MER1_type.DNA.250 [symbol] | 22 | −0.12 | 0.05 | −0.4 | 0.5 | 0.4 | 0.23 | 0.00 |
| 666 | Charlie9.MER1_type.DNA.158 [symbol] | 80 | −0.06 | 0.01 | 0.5 | −0.4 | 0.4 | 0.23 | 0.00 |
| 667 | MSTB1- | 7 | 0.18 | 0.19 | −0.1 | 1.1 | −0.1 | 0.23 | 0.01 |
| 668 | U4.snRNA.snRNA.161.[92] | 10 | −0.06 | −0.05 | −0.4 | 0.5 | 0.5 | 0.23 | 0.00 |
| 669 | MER45R.Tip100.DNA.260.[525 [symbol] | 31 | 0.15 | 0.18 | 1.4 | −0.7 | −0.2 | 0.21 | 0.00 |
| 670 | HERV35I.ERV1.LTR.426.[594] | 35 | 0.10 | 0.16 | 1.2 | −0.6 | 0.1 | 0.21 | 0.00 |
| 671 | AluSp.Alu.SINE.50469.[287] | 13694 | 0.01 | 0.07 | 0.7 | 0.0 | −0.0 | 0.21 | 0.00 |
| 672 | L1M2a1.L1.LINE.142.[918] | 8 | −0.03 | 0.34 | 1.1 | −0.4 | 0.1 | 0.21 | 0.00 |
| 673 | LTR70.ERV1.LTR.128.[673] | 14 | 0.32 | 0.35 | 2.3 | 0.5 | −1.0 | 0.21 | 0.01 |
| 674 | Charlie7.MER1_type.DNA.472 [symbol] | 290 | −0.04 | 0.05 | 0.0 | 0.0 | 0.3 | 0.21 | 0.00 |
| 675 | MER113.MER1_type.DNA.4474. [symbol] | 363 | −0.07 | −0.05 | 0.8 | −0.0 | 0.5 | 0.21 | 0.01 |
| 676 | body.MER1_type | 14452 | −0.04 | 0.00 | −0.1 | 0.7 | −0.3 | 0.21 | 0.00 |
| 677 | Charlie2.MER1_type.DNA.330 [symbol] | 382 | 0.02 | 0.08 | 1.3 | 0.0 | −0.4 | 0.20 | 0.00 |
| 678 | MER83B- | 10 | −0.05 | 0.02 | −0.4 | 0.7 | 0.0 | 0.20 | 0.00 |
| 679 | AluSx.Alu.SINE.332026.[290 [symbol] | 45649 | −0.01 | 0.03 | 0.9 | −0.2 | 0.2 | 0.19 | 0.00 |
| 680 | MER61C- | 8 | −0.14 | 0.08 | −0.6 | 0.9 | −0.0 | 0.19 | 0.01 |
| 681 | MER20.MER1_type.DNA.16178. [symbol] | 1409 | 0.04 | 0.05 | 1.0 | −0.4 | 0.2 | 0.19 | 0.00 |
| 682 | Charlie2b.MER1_type.DNA.50 [symbol] | 29 | −0.08 | −0.02 | 0.4 | 0.2 | 0.0 | 0.19 | 0.00 |
| 683 | HAL1b.L1.LINE.7227.[255] | 370 | −0.02 | 0.08 | 0.1 | 0.1 | 0.4 | 0.18 | 0.00 |
| 684 | MER68.ERVL.LTR.1892.[361] | 176 | 0.07 | 0.16 | 1.0 | 0.6 | 0.0 | 0.18 | 0.00 |
| 685 | Tigger3b.MER2_type.DNA.499 [symbol] | 444 | −0.04 | 0.10 | 0.3 | 0.4 | 0.0 | 0.18 | 0.00 |
| 686 | MER5C.MER1_type.DNA.1512.[ [symbol] | 102 | −0.09 | −0.03 | 0.4 | 0.7 | −0.4 | 0.18 | 0.00 |
| 687 | MER83B.ERV1.LTR.147.[252] | 21 | −0.12 | 0.00 | −0.8 | 0.7 | 0.4 | 0.17 | 0.01 |
| 688 | LTR19C.ERV1.LTR.213.[410] | 26 | 0.06 | 0.09 | 1.2 | −0.5 | 0.0 | 0.17 | 0.00 |
| 689 | tRNA-Thr- | 3 | 0.27 | 0.13 | −0.9 | −0.1 | 0.5 | 0.17 | 0.01 |
| 690 | tRNA-Ser- | 7 | 0.13 | −0.04 | 0.0 | 0.6 | 0.1 | 0.17 | 0.00 |
| 691 | LTR24C.ERV1.LTR.380.[322] | 31 | −0.04 | 0.08 | 0.2 | 0.5 | 0.2 | 0.17 | 0.00 |
| 692 | body.MuDR | 86 | 0.03 | 0.12 | 1.6 | −0.0 | −0.4 | 0.16 | 0.01 |
| 693 | LTR10C.ERV1.LTR.507.[462] | 51 | 0.12 | 0.09 | 0.7 | 0.7 | −0.4 | 0.16 | 0.01 |
| 694 | LTR49- | 80 | 0.03 | 0.09 | 0.3 | 0.2 | 0.0 | 0.16 | 0.00 |
| 695 | tRNA-Ser- | 1 | 0.60 | 0.39 | 0.6 | −0.5 | 0.2 | 0.15 | 0.00 |
| 696 | MER21C- | 15 | 0.05 | 0.16 | −0.4 | −0.1 | 0.6 | 0.15 | 0.00 |
| 697 | MER121.Unknown.Unknown.919 [symbol] | 54 | −0.01 | −0.01 | −0.6 | 0.8 | 0.1 | 0.14 | 0.00 |
| 698 | FRAM/FAM.Alu.SINE.530.[68] | 48 | 0.07 | 0.06 | −0.2 | −0.3 | 0.6 | 0.14 | 0.00 |
| 699 | MER96B.MER1_type?.DNA.2121 [symbol] | 215 | −0.01 | 0.01 | 0.0 | −0.1 | 0.2 | 0.14 | 0.00 |
| 700 | LTR2-int.ERV1.LTR.5.[222] | 1 | −0.43 | −0.29 | 0.6 | 0.8 | −0.3 | 0.14 | 0.02 |
| 701 | body.Merlin | 1 | −0.38 | −0.64 | 0.0 | −0.2 | 0.4 | 0.14 | 0.00 |
| 702 | tRNA-Glu- | 17 | −0.21 | −0.29 | 1.5 | −1.0 | 0.2 | 0.13 | 0.01 |
| 703 | MER31B.ERV1.LTR.1459.[309] | 111 | 0.02 | 0.05 | 0.7 | 0.3 | −0.4 | 0.13 | 0.00 |
| 704 | Charlie2a.MER1_type.DNA.16 [symbol] | 113 | 0.04 | 0.11 | −0.4 | 0.5 | 0.3 | 0.13 | 0.00 |
| 705 | MER63B.AcHobo.DNA.544.[368 [symbol] | 28 | 0.13 | 0.14 | 0.8 | −1.9 | 0.6 | 0.13 | 0.03 |
| 706 | MER117.MER1_type.DNA.4409. [symbol] | 192 | −0.01 | 0.02 | −0.3 | 0.6 | 0.2 | 0.13 | 0.00 |
| 707 | body.MER1_type? | 406 | 0.02 | 0.04 | 0.9 | −0.7 | 0.5 | 0.13 | 0.00 |
| 708 | MER61F.ERV1.LTR.148.[438] | 21 | 0.28 | 0.37 | 1.4 | −0.8 | −0.0 | 0.13 | 0.01 |
| 709 | MER41B- | 33 | −0.18 | 0.01 | −0.1 | 0.2 | 0.0 | 0.13 | 0.00 |
| 710 | AluJ/FLAM.Alu.SINE.3444.[7 [symbol] | 313 | −0.06 | −0.04 | 0.6 | 0.0 | −0.1 | 0.13 | 0.00 |
| 711 | MER5B.MER1_type.DNA.24584. [symbol] | 1582 | −0.03 | 0.01 | 0.0 | 0.0 | 0.2 | 0.12 | 0.00 |
| 712 | MLT1F2- | 6 | −0.03 | −0.00 | −0.8 | −0.4 | 0.4 | 0.12 | 0.01 |
| 713 | MIRm.MIR.SINE.32156.[90] | 2084 | −0.01 | −0.02 | −0.4 | 0.4 | 0.1 | 0.12 | 0.00 |
| 714 | AluYc3.Alu.SINE.1758.[58] | 180 | 0.05 | 0.08 | 1.1 | −0.5 | −0.6 | 0.12 | 0.00 |
| 715 | LTR12C- | 5 | −0.13 | 0.03 | 0.4 | 0.8 | −0.5 | 0.12 | 0.00 |
| 716 | LTR10B1- | 12 | 0.33 | 0.26 | 1.3 | −0.6 | −0.2 | 0.11 | 0.00 |
| 717 | tRNA- | 5 | −0.03 | −0.16 | 0.0 | 0.6 | −0.1 | 0.11 | 0.00 |
| 718 | Charlie1b.MER1_type.DNA.19 [symbol] | 163 | −0.11 | −0.06 | −0.4 | 0.5 | −0.3 | 0.11 | 0.00 |
| 719 | AluJo/FLAM.Alu.SINE.2189.[ [symbol] | 270 | −0.09 | −0.07 | 0.6 | −0.3 | 0.3 | 0.11 | 0.01 |
| 720 | MER96.MER1_type?.DNA.1232. [symbol] | 282 | 0.01 | 0.03 | −0.1 | 0.5 | 0.0 | 0.11 | 0.00 |
| 721 | MER45.Tip100.DNA.678.[86] | 501 | 0.04 | 0.05 | 0.7 | −0.2 | 0.1 | 0.10 | 0.00 |
| 722 | Looper.PiggyBac.DNA.514.[3 [symbol] | 38 | −0.13 | −0.02 | −0.1 | 0.6 | −0.4 | 0.10 | 0.00 |
| 723 | FLAM_A.Alu.SINE.17781.[117 [symbol] | 1265 | 0.00 | 0.02 | 0.7 | 0.0 | −0.2 | 0.10 | 0.00 |
| 724 | Zaphod2.Tip100.DNA.715.[19 [symbol] | 45 | −0.03 | 0.01 | 1.0 | −0.1 | −0.1 | 0.10 | 0.00 |
| 725 | LTR12E.ERV1.LTR.127.[781] | 85 | 0.21 | 0.25 | 0.8 | −0.4 | 0.1 | 0.10 | 0.01 |
| 726 | MLT1G- | 8 | 0.20 | 0.34 | 0.7 | −0.6 | −0.1 | 0.10 | 0.00 |
| 727 | MER85.PiggyBac.DNA.892.[12 [symbol] | 39 | −0.07 | 0.04 | −0.3 | 0.1 | 0.2 | 0.09 | 0.00 |
| 728 | MER34B- | 31 | −0.13 | 0.09 | −0.2 | 0.5 | 0.2 | 0.09 | 0.00 |
| 729 | MER4D1- | 4 | −0.09 | 0.16 | 0.2 | −0.2 | 0.4 | 0.09 | 0.00 |
| 730 | MER69B.Tip100.DNA.478.[511 [symbol] | 41 | −0.01 | 0.09 | −0.1 | −0.4 | 0.2 | 0.08 | 0.00 |
| 731 | MER4E- | 9 | 0.05 | 0.20 | 0.1 | −0.2 | 0.3 | 0.08 | 0.00 |
| 732 | LTR24B- | 7 | 0.14 | 0.17 | 0.1 | −0.1 | −0.1 | 0.08 | 0.00 |
| 733 | BSR/Beta.Satellite.Satelli [symbol] | 48 | −0.02 | 0.10 | 1.2 | −0.5 | 0.0 | 0.08 | 0.00 |
| 734 | AluSp/q.Alu.SINE.2568.[100 [symbol] | 283 | 0.09 | 0.11 | 1.4 | −0.6 | −0.0 | 0.08 | 0.00 |
| 735 | L1M3f.L1.LINE.1234.[941] | 77 | 0.15 | 0.33 | 1.8 | −0.8 | −0.7 | 0.08 | 0.00 |
| 736 | tRNA-Arg- | 2 | 0.39 | 0.49 | 0.0 | −0.2 | 0.2 | 0.08 | 0.01 |
| 737 | Harlequin.ERV1.LTR.610.[15 [symbol] | 129 | 0.33 | 0.46 | 1.0 | −0.0 | −0.6 | 0.07 | 0.00 |
| 738 | HERV15.ERV1.LTR.116.[1879] | 27 | −0.04 | −0.09 | −0.9 | 0.3 | 0.3 | 0.07 | 0.00 |
| 739 | LTR1D- | 2 | 0.60 | 0.82 | 0.4 | −0.3 | 0.3 | 0.07 | 0.00 |
| 740 | body.RNA | 1109 | −0.03 | −0.03 | −0.4 | −0.4 | 0.5 | 0.07 | 0.00 |
| 741 | LTR65.ERV1.LTR.452.[268] | 34 | 0.12 | 0.07 | 0.7 | 0.0 | −0.3 | 0.07 | 0.00 |
| 742 | PABL_A- | 21 | −0.09 | 0.08 | −0.5 | 0.6 | −0.0 | 0.07 | 0.00 |
| 743 | MER65B- | 7 | −0.15 | −0.01 | −1.1 | 0.6 | −0.0 | 0.06 | 0.01 |
| 744 | MER4E1- | 9 | 0.04 | 0.18 | 0.1 | −0.4 | 0.0 | 0.06 | 0.00 |
| 745 | LTR13.ERVK.LTR.458.[836] | 327 | 0.25 | 0.29 | 1.0 | −0.8 | −0.0 | 0.06 | 0.00 |
| 746 | MER84.ERV1.LTR.345.[286] | 43 | −0.05 | 0.05 | 0.4 | 0.4 | −0.2 | 0.05 | 0.00 |
| 747 | MER21A- | 21 | −0.08 | 0.06 | −0.7 | 0.5 | 0.0 | 0.05 | 0.00 |
| 748 | tRNA-Gln- | 3 | 0.26 | 0.19 | 0.0 | 0.1 | 0.0 | 0.05 | 0.00 |
| 749 | MLT1F1- | 7 | 0.02 | −0.12 | −0.4 | 0.3 | 0.1 | 0.05 | 0.01 |
| 750 | tRNA-Thr- | 2 | −0.04 | −0.23 | 0.1 | 0.4 | −0.1 | 0.05 | 0.00 |
| 751 | Tigger5b.MER2_type.DNA.775 [symbol] | 71 | −0.10 | −0.03 | 0.6 | 0.4 | −0.4 | 0.04 | 0.00 |
| 752 | HERVE.ERV1.LTR.346.[2130] | 192 | −0.04 | −0.08 | −0.2 | 0.2 | 0.2 | 0.04 | 0.00 |
| 753 | LTR27- | 3 | −0.06 | 0.11 | 1.0 | −0.7 | −0.0 | 0.03 | 0.00 |
| 754 | MER95.ERV.LTR.154.[264] | 7 | −0.18 | −0.05 | −0.7 | 0.4 | 0.2 | 0.03 | 0.00 |
| 755 | MER41D- | 5 | 0.09 | 0.15 | 0.6 | −0.7 | 0.3 | 0.03 | 0.01 |
| 756 | MER91.Tip100.DNA.553.[55] | 364 | 0.04 | 0.03 | 0.4 | 0.0 | −0.2 | 0.02 | 0.00 |
| 757 | tRNA-Gly- | 12 | −0.12 | −0.10 | 0.4 | 0.4 | −0.1 | 0.02 | 0.00 |
| 758 | HERVK14C.ERVK.LTR.81.[1701 [symbol] | 13 | 0.01 | 0.13 | 0.4 | 0.3 | −0.1 | 0.02 | 0.00 |
| 759 | MER39B- | 7 | 0.22 | 0.33 | 1.0 | 0.4 | −0.7 | 0.01 | 0.01 |
| 760 | body.scRNA | 129 | −0.06 | −0.02 | −0.3 | 0.2 | 0.0 | 0.01 | 0.00 |
| 761 | MER4B- | 63 | −0.02 | 0.12 | −0.2 | 0.0 | 0.3 | 0.01 | 0.00 |
| 762 | MER97b.MER1_type?.DNA.194. [symbol] | 18 | −0.09 | −0.01 | 0.0 | 0.3 | −0.1 | 0.01 | 0.00 |
| 763 | LTR8-int.ERV1.LTR.53.[752] | 4 | −0.18 | −0.05 | 0.5 | −0.1 | 0.0 | 0.01 | 0.00 |
| 764 | L3.CR1.LINE.47228.[197] | 2942 | −0.03 | 0.02 | −0.2 | 0.4 | −0.1 | 0.01 | 0.00 |
| 765 | U17.snRNA.snRNA.9.[174] | 4 | −0.14 | −0.26 | 0.0 | 0.4 | −0.3 | 0.01 | 0.00 |
| 766 | LTR71B.ERV1.LTR.209.[426] | 28 | 0.03 | 0.03 | −0.0 | −0.4 | 0.1 | 0.00 | 0.00 |
| 767 | Charlie12.MER1_type.DNA.16 [symbol] | 17 | 0.12 | 0.17 | 0.6 | −0.8 | 0.2 | −0.01 | 0.00 |
| 768 | ERVL-C2.ERVL.LTR.54.[807] | 4 | −0.22 | 0.11 | −0.2 | 0.6 | −0.2 | −0.01 | 0.01 |
| 769 | MER97c.MER1_type?.DNA.1428 [symbol] | 91 | 0.07 | 0.10 | 0.6 | −0.8 | −0.2 | −0.01 | 0.00 |
| 770 | MER110A- | 2 | 0.34 | 0.43 | 0.8 | −0.5 | −0.4 | −0.02 | 0.00 |
| 771 | LTR13A.ERVK.LTR.182.[795] | 98 | 0.16 | 0.19 | 0.0 | −0.1 | 0.0 | −0.02 | 0.00 |
| 772 | FRAM.Alu.SINE.21803.[149] | 1895 | −0.02 | 0.01 | 0.0 | −0.1 | 0.0 | −0.02 | 0.00 |
| 773 | TAR1.telo.Satellite.123.[4 [symbol] | 40 | −0.01 | 0.02 | 0.8 | −0.2 | −0.3 | −0.02 | 0.00 |
| 774 | LTR3B_.ERVK.LTR.245.[336] | 26 | 0.18 | 0.24 | 0.3 | 0.2 | −0.4 | −0.02 | 0.00 |
| 775 | LTR68.ERV1.LTR.341.[293] | 9 | 0.05 | 0.22 | 0.4 | −0.7 | 0.1 | −0.04 | 0.00 |
| 776 | U8.snRNA.snRNA.30.[110] | 1 | −0.26 | −0.11 | −0.2 | 0.7 | −0.4 | −0.05 | 0.00 |
| 777 | AluSq.Alu.SINE.93297.[286] | 16445 | −0.02 | 0.03 | 0.4 | −0.7 | 0.0 | −0.05 | 0.00 |
| 778 | MER83- | 5 | 0.02 | 0.19 | 0.1 | −0.6 | 0.4 | −0.05 | 0.00 |
| 779 | GSATX.centr.Satellite.58.[ [symbol] | 25 | −0.12 | −0.12 | 0.4 | −0.0 | −0.2 | −0.06 | 0.00 |
| 780 | LTR26B.ERV1.LTR.436.[324] | 39 | 0.10 | 0.13 | 1.4 | −0.6 | −0.5 | −0.06 | 0.00 |
| 781 | MER67B.ERV1.LTR.438.[353] | 57 | 0.09 | 0.07 | −0.4 | −1.1 | 0.4 | −0.06 | 0.01 |
| 782 | ERVL- | 4 | 0.21 | 0.24 | 0.4 | −0.6 | 0.3 | −0.06 | 0.00 |
| 783 | HERVFH19.ERV1.LTR.135.[947 [symbol] | 20 | 0.24 | 0.21 | 0.1 | −0.2 | −0.4 | −0.06 | 0.00 |
| 784 | ERVL-F.ERVL.LTR.459.[601] | 20 | 0.08 | 0.19 | 1.1 | −0.5 | −0.2 | −0.07 | 0.00 |
| 785 | MLT1E1A- | 2 | −0.10 | 0.02 | 0.0 | 0.6 | −0.4 | −0.07 | 0.00 |
| 786 | MER106B.MER1_type.DNA.839. [symbol] | 67 | 0.07 | 0.10 | 1.0 | −0.0 | −0.6 | −0.07 | 0.00 |
| 787 | AluJ/monomer.Alu.SINE.329. [symbol] | 21 | 0.06 | 0.09 | 0.4 | 0.1 | −0.3 | −0.08 | 0.00 |
| 788 | MER70B.ERVL.LTR.497.[302] | 23 | −0.09 | 0.10 | −0.6 | 0.0 | 0.0 | −0.09 | 0.00 |
| 789 | tRNA-Met- | 2 | −0.28 | −0.28 | 0.4 | −0.6 | −0.3 | −0.10 | 0.00 |
| 790 | LTR52- | 23 | −0.04 | 0.00 | 0.4 | −0.0 | −0.3 | −0.11 | 0.00 |
| 791 | MER65A.ERV1.LTR.1296.[314] | 139 | −0.08 | −0.00 | −0.5 | 0.6 | −0.1 | −0.11 | 0.00 |
| 792 | ALR/Alpha.centr.Satellite. [symbol] | 40 | −0.23 | −0.08 | −0.8 | 0.9 | −0.4 | −0.11 | 0.01 |
| 793 | MLT1E1- | 3 | −0.01 | 0.22 | 0.6 | −0.3 | −0.3 | −0.11 | 0.00 |
| 794 | LTR10E- | 2 | 0.49 | 0.63 | 0.5 | −0.5 | −0.2 | −0.12 | 0.00 |
| 795 | body.DNA | 24141 | −0.03 | 0.03 | 0.0 | −0.0 | −0.3 | −0.12 | 0.00 |
| 796 | LTR10B- | 1 | 0.21 | 0.47 | 0.8 | −0.5 | −0.7 | −0.12 | 0.00 |
| 797 | MLT1G3.MaLR.LTR.596.[291] | 51 | −0.01 | 0.02 | 0.4 | 0.4 | −0.3 | −0.12 | 0.00 |
| 798 | body.MER93B-int | 9 | −0.24 | −0.08 | 0.0 | −0.1 | −0.2 | −0.12 | 0.00 |
| 799 | MER51E.ERV1.LTR.240.[301] | 27 | −0.10 | −0.06 | 0.6 | −0.5 | −0.0 | −0.12 | 0.00 |
| 800 | tRNA-Ser- | 7 | 0.18 | 0.07 | 0.1 | −0.1 | −0.2 | −0.13 | 0.01 |
| 801 | U6.snRNA.snRNA.1666.[87] | 127 | −0.04 | 0.01 | −0.3 | −0.4 | −0.1 | −0.13 | 0.00 |
| 802 | LTR42.ERVL.LTR.287.[361] | 27 | 0.04 | 0.08 | −0.4 | −0.1 | −0.3 | −0.13 | 0.00 |
| 803 | MER51C.ERV1.LTR.180.[396] | 28 | 0.05 | 0.06 | 0.8 | −0.6 | 0.0 | −0.13 | 0.00 |
| 804 | MER51A- | 11 | 0.17 | 0.17 | 0.7 | −0.5 | −0.2 | −0.14 | 0.01 |
| 805 | MER44D.MER2_type.DNA.334.[ [symbol] | 30 | −0.15 | −0.05 | 0.0 | −0.2 | −0.3 | −0.14 | 0.00 |
| 806 | MER7D.MER2_type.DNA.397.[1 [symbol] | 16 | 0.06 | 0.11 | 0.7 | −0.5 | −0.2 | −0.14 | 0.00 |
| 807 | MER99.DNA.DNA.277.[347] | 12 | −0.14 | −0.04 | 0.7 | −0.4 | −0.1 | −0.15 | 0.01 |
| 808 | MIR3.MIR.SINE.70044.[121] | 4408 | −0.01 | −0.03 | 0.0 | −0.6 | 0.2 | −0.15 | 0.00 |
| 809 | MER97a.MER1_type?.DNA.237. [symbol] | 16 | 0.05 | 0.09 | 0.6 | −0.7 | −0.2 | −0.15 | 0.00 |
| 810 | MER104.Tc2.DNA.2013.[149] | 101 | 0.01 | 0.03 | −0.4 | 0.3 | −0.2 | −0.16 | 0.00 |
| 811 | tRNA-Arg- | 3 | −0.24 | −0.37 | 0.0 | −0.5 | −0.1 | −0.16 | 0.00 |
| 812 | tRNA-Thr- | 5 | −0.26 | −0.08 | −0.5 | 0.3 | −0.4 | −0.16 | 0.00 |
| 813 | tRNA-Arg- | 6 | 0.04 | −0.17 | 0.0 | 0.0 | −0.3 | −0.17 | 0.00 |
| 814 | tRNA-Gly- | 7 | −0.12 | −0.04 | 0.0 | 0.0 | −0.3 | −0.17 | 0.00 |
| 815 | LTR60- | 4 | −0.01 | −0.00 | 0.0 | −0.2 | −0.3 | −0.17 | 0.00 |
| 816 | MER51B- | 30 | −0.01 | 0.21 | −0.7 | 1.0 | −0.7 | −0.17 | 0.00 |
| 817 | MER41E.ERV1.LTR.355.[382] | 36 | −0.09 | 0.02 | −0.4 | 0.2 | −0.3 | −0.17 | 0.01 |
| 818 | MER65D- | 1 | −0.69 | −0.66 | 0.4 | −0.3 | −0.4 | −0.18 | 0.00 |
| 819 | BC200.scRNA.scRNA.253.[134 [symbol] | 24 | 0.00 | 0.04 | −0.1 | −0.7 | 0.1 | −0.18 | 0.00 |
| 820 | LTR18A.ERVL.LTR.250.[313] | 30 | 0.06 | 0.10 | 0.1 | −0.1 | −0.2 | −0.18 | 0.00 |
| 821 | MLT2E.ERVL.LTR.1362.[250] | 70 | 0.05 | 0.10 | −0.3 | −0.4 | 0.3 | −0.18 | 0.00 |
| 822 | MER31B- | 14 | −0.06 | 0.13 | −1.1 | −0.3 | 0.3 | −0.18 | 0.00 |
| 823 | MER93b- | 9 | −0.24 | −0.08 | −0.2 | −0.4 | −0.5 | −0.19 | 0.00 |
| 824 | MER52- | 3 | −0.39 | −0.33 | 0.8 | −0.6 | −0.1 | −0.19 | 0.00 |
| 825 | MER87- | 3 | 0.17 | 0.33 | 0.0 | −0.0 | −0.5 | −0.19 | 0.00 |
| 826 | MER101B.ERV1.LTR.239.[304] | 14 | 0.00 | 0.08 | −0.1 | 0.0 | −0.3 | −0.19 | 0.00 |
| 827 | tRNA-Leu- | 3 | 0.12 | −0.09 | −0.4 | −1.0 | 0.3 | −0.19 | 0.00 |
| 828 | tRNA-Val- | 2 | −0.37 | −0.34 | 0.5 | −0.1 | −0.5 | −0.20 | 0.00 |
| 829 | LTR58.ERV1.LTR.83.[344] | 5 | 0.11 | 0.17 | 0.0 | −0.2 | −0.2 | −0.20 | 0.00 |
| 830 | tRNA-Tyr- | 4 | −0.29 | −0.34 | 0.7 | −0.4 | −0.2 | −0.20 | 0.00 |
| 831 | SATR1.Satellite.Satellite. [symbol] | 236 | −0.09 | −0.10 | 0.3 | 0.1 | −0.5 | −0.20 | 0.00 |
| 832 | MER102b.MER1_type.DNA.3930 [symbol] | 271 | −0.02 | 0.00 | 0.0 | −0.4 | 0.0 | −0.20 | 0.00 |
| 833 | Tigger5c.MER2_type.DNA.939 [symbol] | 27 | 0.14 | 0.17 | 1.2 | −0.3 | −0.6 | −0.21 | 0.01 |
| 834 | AluJo/FRAM.Alu.SINE.1339.[ [symbol] | 118 | 0.00 | 0.03 | −0.5 | −0.3 | −0.2 | −0.21 | 0.00 |
| 835 | tRNA-Ala- | 4 | −0.08 | −0.27 | −0.0 | −0.2 | 0.1 | −0.22 | 0.00 |
| 836 | FLAM_C.Alu.SINE.34861.[125 [symbol] | 2632 | −0.05 | −0.03 | 0.4 | −0.3 | −0.5 | −0.22 | 0.00 |
| 837 | AluSq/x.Alu.SINE.7043.[122 [symbol] | 795 | −0.03 | 0.01 | −0.5 | 0.0 | −0.1 | −0.23 | 0.00 |
| 838 | MER52D.ERV1.LTR.576.[932] | 64 | 0.03 | −0.06 | −0.5 | −0.2 | −0.2 | −0.23 | 0.01 |
| 839 | tRNA-Gln- | 2 | −0.52 | −0.63 | −0.3 | −0.5 | −0.4 | −0.23 | 0.00 |
| 840 | tRNA-Glu- | 9 | −0.31 | −0.40 | 0.0 | −0.5 | −0.2 | −0.23 | 0.00 |
| 841 | U7.snRNA.snRNA.246.[54] | 24 | −0.08 | −0.06 | −0.9 | −0.4 | 0.3 | −0.24 | 0.00 |
| 842 | MER67C- | 2 | −0.48 | 0.03 | −0.6 | −0.5 | −0.2 | −0.24 | 0.00 |
| 843 | LTR13_.ERVK.LTR.36.[891] | 123 | 0.17 | 0.20 | −0.2 | −0.3 | −0.4 | −0.24 | 0.00 |
| 844 | LTR24B.ERV1.LTR.457.[293] | 41 | −0.01 | 0.03 | 0.0 | −0.4 | −0.3 | −0.25 | 0.00 |
| 845 | LTR10A- | 1 | 0.68 | 0.38 | −0.6 | −0.8 | −0.2 | −0.25 | 0.00 |
| 846 | HERVK13.ERVK.LTR.64.[1278] | 16 | −0.23 | −0.19 | 0.0 | −0.7 | −0.4 | −0.25 | 0.00 |
| 847 | MER4C.ERV1.LTR.1172.[320] | 101 | −0.00 | 0.06 | −0.0 | −0.4 | −0.4 | −0.26 | 0.00 |
| 848 | AluYb8.Alu.SINE.68.[161] | 15 | −0.01 | 0.03 | 0.0 | −0.4 | −0.3 | −0.26 | 0.00 |
| 849 | LTR9-int.ERV1.LTR.51.[921] | 4 | −0.19 | −0.03 | −0.4 | −0.6 | −0.1 | −0.26 | 0.00 |
| 850 | MER112.MER1_type.DNA.3729. [symbol] | 222 | −0.02 | 0.02 | 0.0 | −0.2 | −0.3 | −0.26 | 0.00 |
| 851 | LTR48- | 8 | −0.05 | 0.02 | −0.4 | −0.4 | −0.2 | −0.27 | 0.00 |
| 852 | body.Satellite | 921 | 0.05 | −0.02 | 0.4 | −0.1 | −0.6 | −0.28 | 0.00 |
| 853 | HERVL- | 4 | 0.69 | 0.65 | 2.9 | −0.4 | −1.9 | −0.29 | 0.04 |
| 854 | LTR19- | 4 | 0.28 | 0.42 | 0.0 | 0.0 | −0.4 | −0.29 | 0.00 |
| 855 | FLAM.Alu.SINE.1401.[62] | 4569 | −0.04 | −0.02 | −0.4 | 0.1 | −0.5 | −0.29 | 0.00 |
| 856 | body.centr | 315 | −0.09 | −0.18 | 0.5 | −0.5 | −0.4 | −0.29 | 0.00 |
| 857 | tRNA-Leu- | 6 | −0.19 | −0.35 | 0.0 | −1.0 | −0.2 | −0.29 | 0.00 |
| 858 | L1P4b.L1.LINE.275.[1316] | 17 | 0.06 | 0.22 | 0.8 | −0.6 | −0.6 | −0.29 | 0.00 |
| 859 | MER2B.MER2_type.DNA.2351.[ [symbol] | 136 | −0.06 | 0.01 | 0.0 | −0.6 | −0.4 | −0.30 | 0.00 |
| 860 | LTR30- | 1 | −0.37 | −0.39 | 0.0 | 0.0 | −0.4 | −0.30 | 0.00 |
| 861 | LTR23- | 16 | −0.00 | −0.07 | −0.4 | −0.8 | −0.3 | −0.30 | 0.00 |
| 862 | Ricksha_b.MuDR.DNA.115.[59 [symbol] | 10 | 0.23 | 0.22 | 0.3 | −0.6 | −0.4 | −0.31 | 0.00 |
| 863 | MER84- | 7 | −0.19 | −0.07 | −0.7 | −0.4 | 0.2 | −0.31 | 0.00 |
| 864 | FordPrefect_a.Tip100.DNA.3 [symbol] | 19 | −0.05 | −0.02 | −0.4 | 0.0 | −0.3 | −0.31 | 0.00 |
| 865 | LTR18B.ERVL.LTR.382.[423] | 35 | 0.07 | 0.02 | 0.4 | 0.1 | −0.7 | −0.31 | 0.00 |
| 866 | tRNA-Cys- | 13 | 0.12 | 0.14 | 0.4 | −0.5 | −0.4 | −0.32 | 0.00 |
| 867 | MER61B- | 12 | −0.15 | −0.08 | 0.5 | −0.4 | −0.6 | −0.33 | 0.00 |
| 868 | L3b.CR1.LINE.7618.[138] | 383 | 0.02 | 0.04 | 0.0 | −0.7 | −0.3 | −0.33 | 0.00 |
| 869 | LTR6A.ERV1.LTR.260.[460] | 73 | 0.09 | 0.08 | 0.0 | −0.1 | −0.6 | −0.36 | 0.00 |
| 870 | LTR6B.ERV1.LTR.149.[506] | 44 | −0.04 | −0.03 | −0.4 | 0.3 | −0.6 | −0.37 | 0.00 |
| 871 | tRNA-Ala- | 1 | 0.07 | −0.29 | 0.0 | −0.7 | −0.4 | −0.37 | 0.00 |
| 872 | MER41C- | 6 | 0.27 | 0.16 | −0.2 | −0.5 | −0.5 | −0.37 | 0.00 |
| 873 | MER68- | 26 | 0.27 | 0.35 | 1.2 | 1.3 | −1.6 | −0.38 | 0.03 |
| 874 | body.MER93b | 89 | −0.05 | −0.00 | −0.8 | 0.1 | −0.7 | −0.38 | 0.00 |
| 875 | LTR61.ERV1.LTR.149.[398] | 16 | −0.16 | −0.15 | −0.5 | −0.6 | −0.4 | −0.41 | 0.00 |
| 876 | 7SLRNA.srpRNA.srpRNA.937.[ [symbol] | 99 | −0.00 | 0.01 | −0.3 | −0.8 | −0.4 | −0.42 | 0.00 |
| 877 | HERVKC4.ERVK.LTR.63.[885] | 13 | −0.14 | −0.06 | 0.5 | −0.1 | −0.8 | −0.43 | 0.00 |
| 878 | LTR29- | 1 | −0.16 | 0.02 | 0.0 | −0.8 | −0.3 | −0.43 | 0.00 |
| 879 | HERVL18.ERVL.LTR.531.[760] | 42 | −0.12 | −0.10 | 0.0 | −0.7 | −0.4 | −0.44 | 0.00 |
| 880 | LTR24- | 4 | −0.17 | 0.01 | −0.4 | −0.1 | −0.6 | −0.45 | 0.00 |
| 881 | MER58D.MER1_type.DNA.282.[ [symbol] | 21 | 0.14 | 0.13 | 0.4 | −0.0 | −0.9 | −0.48 | 0.00 |
| 882 | body.srpRNA | 99 | −0.00 | 0.01 | 0.4 | −0.3 | −0.6 | −0.50 | 0.00 |
| 883 | MER51D.ERV1.LTR.148.[239] | 13 | 0.09 | 0.07 | 0.0 | −0.8 | −0.8 | −0.50 | 0.00 |
| 884 | Kanga1c.Tc2.DNA.186.[374] | 14 | −0.04 | 0.06 | −0.5 | −0.1 | −0.7 | −0.51 | 0.00 |
| 885 | LTR62.ERVL.LTR.312.[425] | 38 | −0.01 | 0.03 | 0.0 | −0.3 | −0.6 | −0.53 | 0.00 |
| 886 | tRNA-Arg- | 6 | −0.26 | −0.33 | 0.0 | −0.8 | −0.6 | −0.54 | 0.06 |
| 887 | tRNA-Asn- | 1 | −0.55 | −0.29 | −0.4 | −0.7 | −0.6 | −0.58 | 0.00 |
| 888 | LTR75.ERVL.LTR.404.[321] | 44 | −0.02 | 0.00 | 0.0 | −0.4 | −0.8 | −0.60 | 0.00 |
| 889 | HERVK11D.ERVK.LTR.57.[1690 [symbol] | 3 | −0.38 | −0.17 | 0.4 | −0.6 | −0.9 | −0.62 | 0.00 |
| 890 | tRNA-Asp- | 9 | 0.19 | −0.01 | −0.7 | −0.6 | −0.8 | −0.62 | 0.01 |
| 891 | L1MB7.L1.LINE.20238.[456] | 1775 | 0.07 | 0.12 | 1.6 | −0.5 | −1.4 | −0.63 | 0.03 |
| 892 | MER4C- | 11 | −0.01 | 0.14 | 0.8 | −0.9 | −1.1 | −0.68 | 0.01 |
| 893 | LTR35.ERV1.LTR.306.[301] | 34 | 0.07 | 0.11 | 0.4 | −0.5 | −1.0 | −0.71 | 0.00 |
| 894 | MER34C_.ERV1.LTR.1038.[282 [symbol] | 72 | −0.02 | −0.01 | 0.1 | 0.1 | −1.1 | −0.73 | 0.01 |
| 895 | LTR10G- | 3 | 0.16 | 0.18 | 0.5 | −1.7 | −0.8 | −0.74 | 0.02 |
| 896 | LTR72B.ERV1.LTR.176.[304] | 19 | −0.01 | 0.08 | −0.6 | −0.3 | −1.0 | −0.75 | 0.01 |
| 897 | LTR39- | 22 | −0.11 | −0.00 | −0.5 | −0.9 | −1.1 | −0.90 | 0.03 |
| 898 | LTR45.ERV1.LTR.179.[297] | 78 | 0.04 | 0.09 | 1.0 | −1.2 | −1.4 | −0.96 | 0.01 |
| 899 | Ricksha_0.MuDR.DNA.99.[617 [symbol] | 4 | −0.42 | −0.09 | −0.2 | −1.2 | −1.5 | −1.09 | 0.01 |
| 900 | LTR6A- | 2 | 0.13 | 0.15 | 0.0 | −1.2 | −1.7 | −1.27 | 0.01 |
| 901 | MER77.ERVL.LTR.2380.[340] | 146 | −0.03 | 0.03 | 0.0 | −1.3 | −2.2 | −1.50 | 0.01 |

[symbol]

indicates data missing or illegible when filed

### B. Example 2

### Loss of Epigenetic Silencing in Tumors Preferentially Affects Primate-Specific Retroelements

1. Introduction

Close to 50% of the human genome harbors repetitive sequences originally derived from mobile DNA elements, and in normal cells this sequence compartment is tightly regulated by epigenetic silencing mechanisms involving chromatin-mediated repression. In cancer cells, repetitive DNA elements suffer abnormal demethylation, with potential loss of silencing. A genome-wide microarray approach was used to measure DNA methylation changes in cancers of the head and neck, and to compare these changes to alterations found in adjacent non-tumor tissues. Specific alterations were observed at thousands of small clusters of CpG dinucleotides associated with DNA repeats. Among the 257,599 repetitive elements probed, 5 to 8% showed disease-related DNA methylation alterations. In dysplasia, a large number of local events of loss of methylation appear in apparently stochastic fashion. Loss of DNA methylation is most pronounced for certain members of the SVA, HERV, LINE-1P, AluY, and MaLR families. The methylation levels of retrotransposons are discretely stratified, with younger elements being highly methylated in healthy tissues, while in tumors these young elements suffer the most dramatic loss of methylation. Wilcoxon test statistics reveal that a subset of primate LINE-1 elements is demethylated preferentially in tumors, as compared to non-tumoral adjacent tissue. Sequence analysis of these strongly demethylated elements reveals genomic loci harboring full-length, as opposed to truncated elements, while possible enrichment for functional LINE-1 ORFs is weaker. This analysis indicates that in non-tumor adjacent tissues there is generalized and highly variable disruption of epigenetic control across the repetitive DNA compartment, while in tumor cells a specific subset of LINE-1 retrotransposons that arose during primate evolution suffers the most dramatic DNA methylation alterations.

Herein is a systematic study of DNA methylation changes occurring in the repetitive DNA compartment of squamous carcinomas of the head and neck. In contrast to previous studies, a novel microarray-based approach to obtain discrete DNA methylation data at hundreds of thousands of individual repetitive DNA loci in the human genome was used. Extensive annotation resources for different subfamilies of repeats was then used to evaluate possible relationships between loss of epigenetic silencing in the context of natural history of cancer, and the evolutionary history of repetitive element sub-compartments in the human genome.

2. Materials and Methods

A specific microarray analysis method permits genome-wide assessment of DNA methylation status using restriction endonucleases (described below). Among the 339,314 probes in the microarray, 257,599 are dedicated to the measurement of the methylation levels of individual members of interspersed DNA repeat families. The probes, and the loci to which they hybridize, can be grouped into families or categories of probes and loci based on, for example, repetitive DNA sequence families to which the loci belong. Such groups can be used as collective status biomarkers.

i. Principle of the DNA Methylation Analysis Method

Multiple displacement amplification (MDA, Dean et al., 2002; Lage et al., 2003; Lage et al., 2005; U.S. Patent Application Publication No. 20040063144) is an isothermal amplification method based on random priming and DNA hyper-branching, catalyzed by a strand-displacing DNA polymerase. The yield of the MBA reaction is strongly influenced by the size of the DNA used as template (Lage et al., 2005). The dependence of amplification yield using DNA templates of different size have been systematically studied, and computational model of the reaction that fits the experimental data was built. The results of this analysis indicate that the yield of DNA derived from any sequence segment depends on template size, and additionally on the distance of the sequence segment from the nearest DNA terminus on the template molecule. Other amplification techniques that have similar effect can also be used. A specific cleavage event in a genomic DNA molecule could be detected by measuring DNA amplification yield using a DNA microarray, and a probe in the microarray would be able to measure a local reduction in sequence representation due to cleavage, even if that cleavage event occurred as far as 1200 bases upstream or downstream from the location of the probe. This property enables the use of probe designs that measure cleavage events not only in unique DNA sequences overlapping a probe, but also cleavage events within repetitive DNA sequences that contain CpG dinucleotides, located in the vicinity of a probe of unique sequence, within a window of approximately 2400 bases surrounding the probe. Experimental data is provided that helps to define the approximate size of the window that enables probing-at-a-distance.

ii. Microarray Probe Design

DNA probes of unique sequence (uniqueness assessed using merEngine, Healy et al., 2003) were designed to map as closely as possible to every CpG island in the human genome. The DNA sequences located within a window of plus or minus 4 kb from loci coding for microRNAs were examined, and many of these regions contained small clusters of CpG residues. A relatively lax “CpG islet” specification was then created, requiring that a region in the genome contain a minimum of 7 CpG residues, that the ratio of the CG count to the GC content be larger than 0.53, and that the region be no shorter than 200 bases to be nominated as a CpG islet (this is only an example of a specification of CpG islets; other specifications are disclosed elsewhere herein). Using this specification, 453 out of the 532 microRNA loci in the Sanger database (Griffiths-Jones, 2006) are associated with at least one CpG islet within a window of +/−4 kb. By contrast, based on the more restrictive Takai and Jones definition (2002), the equivalent count of CpG islands in the vicinity of microRNA loci is 141. The total count of CpG islets in the human genome using this relaxed specification is approximately 500,000. A custom microarray containing probes for all CpG islands and CpG islets was designed, in order not to miss DNA methylation changes that may occur in tumors in CpG-rich regions that would not fit the standard CpG island definition. Five broad classes of CpG islands and CpG islets were probed: promoter associated, unique, non-promoter associated, interspersed repeat associated (Jurka, 1998; Smit, 1996-2004), tandem repeat associated (Benson, 1999), and microRNA locus associated (Griffiths-Jones, 2006). A subset of the probes were replicated on the array surface, bringing the total number of probes in the microarray to 377,000. The coordinates of the probes relevant to the Top 138 repetitive DNA sequence families are shown in Table 15.

Experimental Work Flow for Microarray Analysis

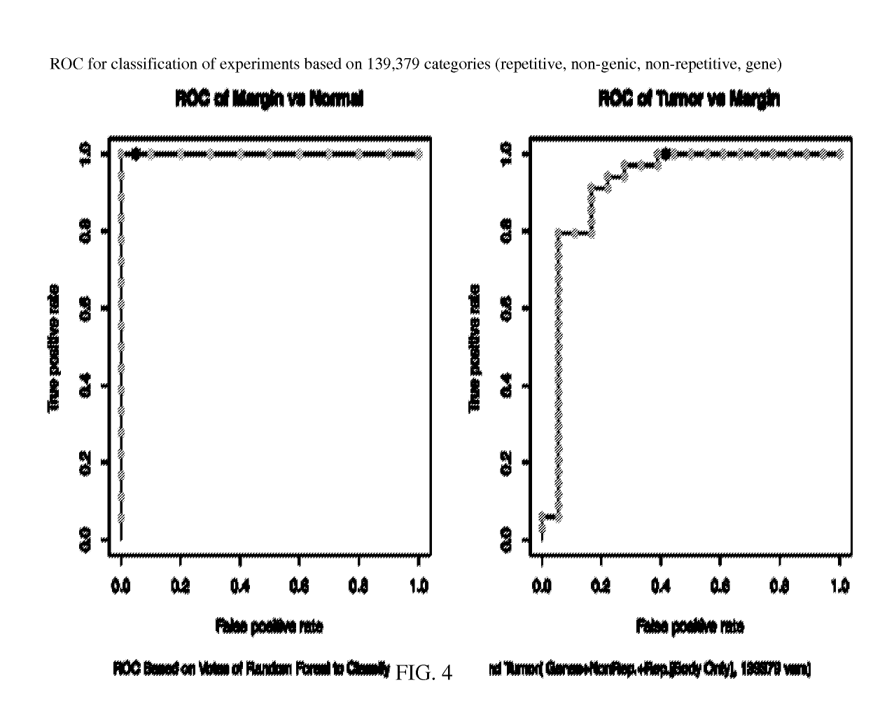

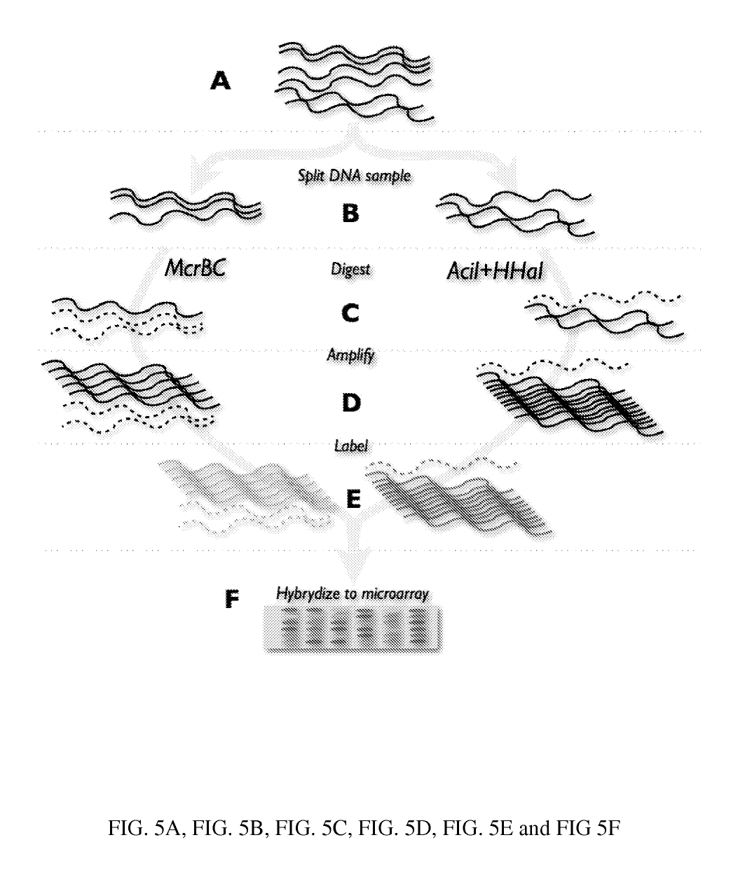

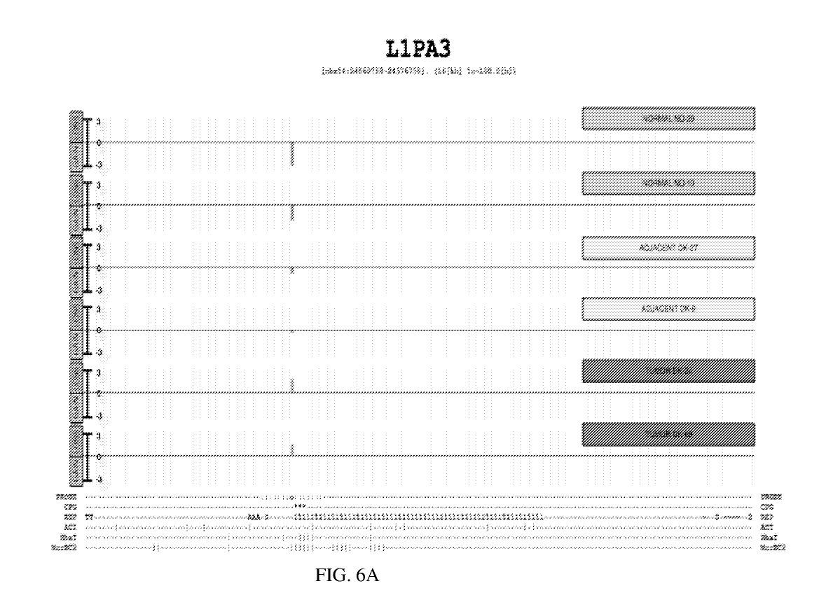

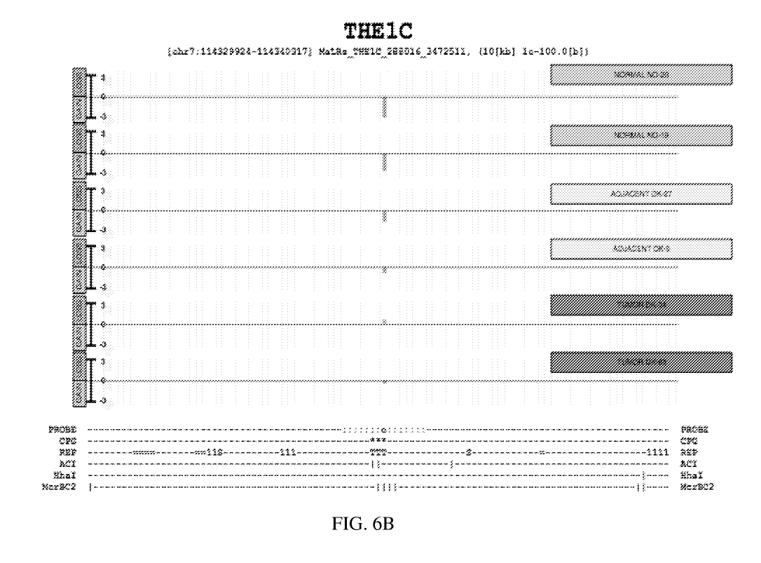

Relative methylation was measured by splitting the DNA sample in two equal aliquots, and digesting each aliquot with either methylation-sensitive or methylation-dependent restriction endonucleases, respectively, as shown diagrammatically in FIG. 5. Each of the two digests was amplified by MDA, and then labeled with a different dye, followed by mixing after labeling, and processed for DNA microarray analysis as described in the Microarray response section below. The enzymes used to sample DNA methylation have fairly high sampling efficiency when used individually, as ascertained using sequence analysis. Table 8 documents the theoretical sampling efficiency of the mixture of the methylation-sensitive endonucleases AciI (recognition site CCGC) and HhaI (recognition site GCGC). The table also documents the sampling efficiency of the methylation-dependent endonuclease McrBC (recognition site Pu^{m}C[N40-3000]Pu^{m}C). Of course, the enzymes do not sample all CpG residues in the genome, but this limitation is alleviated by the fact that most neighboring CpG residues in a CpG island tend to have similar methylation status at any given time. By theoretical sampling efficiency, it is meant that known cleavage sites exist in the sequence within or near a CpG island, which may or may not be methylated in any given DNA sample, but would cause a fluorescence intensity change in either channel of the microarray whenever a methylation change occurred. Since DNA was sampled using two separate digestion reactions, probed loci should be capable of reporting DNA methylation changes based on the presence or absence of cleavage in a single color channel, as well as detecting signal alterations in both color channels, reflecting changes in the combined cleavage susceptibility to the two classes of endonuclease used. The last column on Table 8 indicates that if one considers theoretical cleavage sites for both sets of enzymes in combination, the potential sampling efficiency increases to 99.9% of all probed CpG islands. The labeled DNA is hybridized to the custom microarray, and subsequently the ratio of intensities is generated for locus-specific methylation levels associated with each probe. In order to illustrate the relationship between the probe location and the location of restriction endonuclease sites in CpG rich domains associated with specific repetitive elements, two detailed maps of interspersed repetitive elements are shown that were probed in the microarray. FIG. 6A shows a map of a LINE-1PA3 element that was probed using a unique sequence located within 150 bases of the 5′-terminus of the retroelement. The Figure shows the location of the CpG islet in the retroelement, as well as the location of all possible restriction endonuclease sites within and around the element. FIG. 6B shows a similar map, in this case corresponding to a THE1C element.

Analysis with ASCIIMap can show the locations of the probes and the restriction endonuclease cutting sites in the CpG islands associated with these elements. Probes for unique sequences can overlap with repetitive DNA sequences. The probe design algorithm always ensures that the sequence is unique before designing a probe. The apparent paradox that a repetitive element may have parts that are unique sequences can be explained by considering the age of the repetitive elements for which the probe is designed. For example, an element of the family MLT1C, 85 MYO: over a span of millions of years since it appeared in its original form in the genome, its sequence have deteriorated from its consensus so much that although the element can still be classified as MLT1C now (based on the overall structure and certain sequence patterns), its sequences acquired enough random mutations that the probe algorithm can recognize certain parts within this MLT1C as unique in the genome. For repetitive element families that are younger, i.e. the elements that haven't had evolutionary time to acquire mutations differentiating them from their respective consensus, the probe designer most likely designs the probe within the 100 bases flanking region of the repetitive element. Conversely, for the older repetitive elements (20, 30, 40+ MYO), the probe designer is able to find regions that have uniquely diverged from the global consensus of the repeat family.

TABLE 8
Enzymes' efficiency at sampling CpG islands.

| | | | | |
|---|---|---|---|---|
| CpG island near: | sampled | [%] | failed | [%] |

AciI & HhaI

| | | | | |
|---|---|---|---|---|
| Gene | 22065 | 99.84% | 36 | 0.16% |
| LINE | 34297 | 99.44% | 193 | 0.56% |
| SINE | 178849 | 99.45% | 993 | 0.55% |
| LTR | 22588 | 99.53% | 106 | 0.47% |
| Satellite | 993 | 99.50% | 5 | 0.50% |
| DNA | 8411 | 99.60% | 34 | 0.40% |
| Other repetitive | 3988 | 99.97% | 1 | 0.03% |
| element | | | | |
| tandem repeat | 674 | 88.68% | 86 | 11.32% |
| Unique, unclassified | 65947 | 99.38% | 409 | 0.62% |
| genomic sequence | | | | |
| Total CpG Islands | 337812 | 99.42% | 1863 | 0.58% |

McrBC[40-3000]

| | | | | |
|---|---|---|---|---|
| Gene | 22100 | 100.00% | 1 | 0.00% |
| LINE | 34485 | 99.99% | 5 | 0.01% |
| SINE | 179818 | 99.99% | 24 | 0.01% |
| LTR | 22692 | 99.99% | 2 | 0.01% |
| Satellite | 998 | 100.00% | 0 | 0.00% |
| DNA | 8441 | 99.95% | 4 | 0.05% |
| Other repetitive | 3989 | 100.00% | 0 | 0.00% |
| element | | | | |
| tandem repeat | 750 | 98.68% | 10 | 1.32% |
| Unique, | 66338 | 99.97% | 18 | 0.03% |
| unclassified | | | | |
| genomic sequence | | | | |
| Total CpG Islands | 339611 | 99.98% | 64 | 0.02% |

AciI & HhaI plus McrBC

| | | | | |
|---|---|---|---|---|
| Gene | 22101 | 100.00% | 0 | 0.00% |
| LINE | 34487 | 99.99% | 3 | 0.01% |
| SINE | 179840 | 100.00% | 2 | 0.00% |
| LTR | 22693 | 100.00% | 1 | 0.00% |
| Satellite | 998 | 100.00% | 0 | 0.00% |
| DNA | 8443 | 99.98% | 2 | 0.02% |
| Other repetitive | 3989 | 100.00% | 0 | 0.00% |
| element | | | | |
| tandem repeat | 756 | 99.47% | 4 | 0.53% |
| Unique, unclassified | 66350 | 99.99% | 6 | 0.01% |
| genomic sequence | | | | |
| Total CpG Islands | 339657 | 99.99% | 18 | 0.01% |

The table divides the CpG islands based on the type of genomic elements with which they are overlapping.
The “sampled” columns show how many CpG islands in a given group contain at least one enzyme recognition motif i.e. theoretically, how many members of the group of CpG islands will a specific endonuclease be able to cleave.
The columns labeled “[%]” which immediately follow the “sampled” columns, show the percentage of all CpG islands belonging to a specific sub group that have at least one enzyme recognition site, or showing the theoretical capacity or efficiency for an enzyme to cleave the sequences of the particular sub-category of CpG islands.
Analogously, the “failed” columns show how many CpG islands do not contain the enzyme recognition motif, which is shown in relative terms in the column labeled [%] immediately following the “failed” column.
The ability of enzymes to cleave particular subgroups of CpG island in the “sampled” colums, i.e. the sampling efficiency for an enzyme, is shown with respect to AciI and HHaI enzymes (used in a single buffer, and thus the numbers in “sampled” columns indicate whether a CpG island contained either AciI or HHaI motif), McrBC, and, collectively, for all three enzymes.

The experimental data obtained from 74 different probe loci in the microarray was independently validated by bisulfite sequencing using either Sanger sequencing of individual clones of PCR products, or using the Sequenom EpiTyper platform, which is based on sequencing of transcribed RNA by mass spectrometry. Sanger-based analysis was performed for a total of 59 different microarray probes. The correlation between the microarray read-out and the results of Sanger sequencing was analyzed based on the count of CpGs methylated or demethylated in all the clones of the sequencing result of a locus, the sequences were classified as un-methylated, composite or methylated. For 48 of the 59 probes, there was agreement between the microarray methylation result and the bisulfite Sanger sequencing result, for a concordance of 81.4%. The bisulfite sequencing validation analysis produced methylation results from the microarray analysis, the map position of the probes, and the bisulfite sequencing result for one gene promoter region, one AluSq element and one AluY element. In these three validation experiments there is agreement between the microarray result and the bisulfite sequencing data. It should be noted, however, that in the case of the probe that samples the AluSq element there are neighboring sequences belonging to an MLT1C LTR element, whose methylation status will influence the measurements. An effort was made, through extensive probe annotation data, to keep track of these complex cases. The results presented herein were generated by calculating the average methylation of hundreds, or even thousands of repetitive elements belonging to specific families of repeats. This averaging process will minimize the influence of the surrounding sequence context.

iv. Specimen Sample Acquisition and DNA Preparation

Tumor samples and adjacent non-tumor tissue were obtained through the Tissue Procurement Program of the Surgical Pathology Laboratory at Yale New Haven Hospital. All patients provided informed consent (IRB/HIC #14414). Representative histological sections of all specimens were reviewed to confirm the nature of the sample. After informed consent, oral epithelial cells from subjects with no known risk for oral cancer were obtained by scraping. DNA from all tissues was obtained using MasterPure DNA Purification Kit (EPICENTRE). The protocol follows: for every reaction a mix of 150 μL of Tissue and Cell Lysis solution and 1.5 μL of proteinase K from the kit was created. Lysate from about 8 mm³ of specimen was collected. The lysate was vortexed every 5 min until the tissue was completely dissolved. The incubation at 65 degrees followed for 30-60 min. Subsequently 0.5 μL of RNase was added to each tube and incubated for 30 min at 37 degrees. 75 μL of MPC protein precipitation agent was added to the lysed sample. After centrifugation for 10 min at 15,000 rpm the supernatant was transferred to a labeled 1.5 mL tube. With 250 μL of isopropanol added to the supernatant the tube was inverted multiple times. The DNA was then transferred using Pasteur pipet and resuspended in 100 μL of TE (0.1 mM EDTA). The DNA was then stored for 2 days at 4 degrees. Subsequent quantitation was done using PicoGreen fluorescence.

200 ng genomic DNA extracted from the head and neck tumor or the corresponding non-tumor adjacent tissues were digested by two sets of restriction enzymes respectively. One genomic sample was digested by McrBC (New England Biolabs), the other was digested by AciI and HhaI (New England Biolabs). 20 units of each enzyme were used to set up 45 μl reaction in the recommended buffer (McrBC: 50 mM NaCl, 10 mM Tris-HCl, 10 mM MgCl2, 1 mM DTT supplemented with 100 μg/mL BSA, and 5 mM GTP; AciI and Hha: 50 mM Tris-HCl, 100 mM NaCl, 10 mM MgCl2, 1 mM DTT supplemented with 100 μg/mL BSA). Reactions were incubated at 37 C for 6 hours and then boosted with an additional 10 units of the corresponding enzyme for another 12 hrs, and finally inactivated at 65 C for 20 minutes. One aliquot of each digested genomic DNA (20 ng) was subjected to whole genome amplification respectively using REPLI-G kit (Qiagen) with 8 hours incubation at 30 C. The amplified DNA sample was then purified by QIAEX II kit (Qiagen) with slightly modified protocol (3 instead of 2 washes with PE buffer and finally eluted in water rather than EB buffer). 4 μg of the purified genomic DNA sample was submitted to Nimblegen for labeling and hybridization.

v. Microarray Response

A control experiment defined the longest distance from a probe at which endonuclease cuts can be measured using the microarray method. DNA was cleaved with PmeI, and processed by isothermal whole genome amplification followed by microarray analysis using uncleaved, amplified DNA as a reference channel. All probes containing a single predicted PmeI cut, and not bounded by another cut within a distance of +−40 kb were plotted at x=0 in an xy plot. Other probes proximal to the cut site (upstream as well as downstream) were plotted according to their position in the x axis. The ratio of the two microarray channels (cleaved and uncleaved DNA) was plotted in the y axis. The deflection of the y axis in the xy plot indicates that a single endonuclease cut produces large changes in the ratio (y) within a window of +/−3.0 kb, with the most pronounced deflection of the ratio occurring within a window of +/−1.2 kb.

vi. Validation of Microarray-Based Observations Using Bisulfite DNA Sequencing

a total of 74 probe loci that showed DNA methylation changes in tumors were selected, and the DNA methylation status was examined using bisulfite DNA sequencing across a total of 12 experiments, for a total of 207 probe validation data points. DNA sequencing was performed using two different experimental approaches. In the first approach, bisulfite-treated DNA was used to amplify by PCR the genomic regions of interest, and the PCR amplicons were cloned. Individual clones were processed for Sanger sequencing in both strand orientations. In the second approach, bisulfite-treated DNA was used to amplify by PCR the genomic regions of interest, and the PCR amplicons were then transcribed to generate complementary DNA using reagents provided by Sequenom, Inc. as part of their EpiTYPER kit. The RNA was then cleaved with ribonuclease A, and subjected to mass spectrometry analysis. Using software provided by Sequenom, the mass spectrograms were processed to generate a fractional value of DNA methylation between 0.0 and 1.0. When multiple probes associated with a single CpG island were averaged, the concordance of the microarray calls and the bisulfite sequencing results was 87.6%.

vii. Plotting the Data

All members of each phylogenetic branch of the repetitive element subfamilies were grouped together based on their sequence homology and estimated evolutionary age. The average of the individual log 2 methylation values were calculated for all microarray probes belonging to each branch, and plotted these subset-specific values across all experiments. A fourth class of experiments included three technical replicates of a microarray analysis performed using DNA from human sperm.

a. Per-Experiment Plots

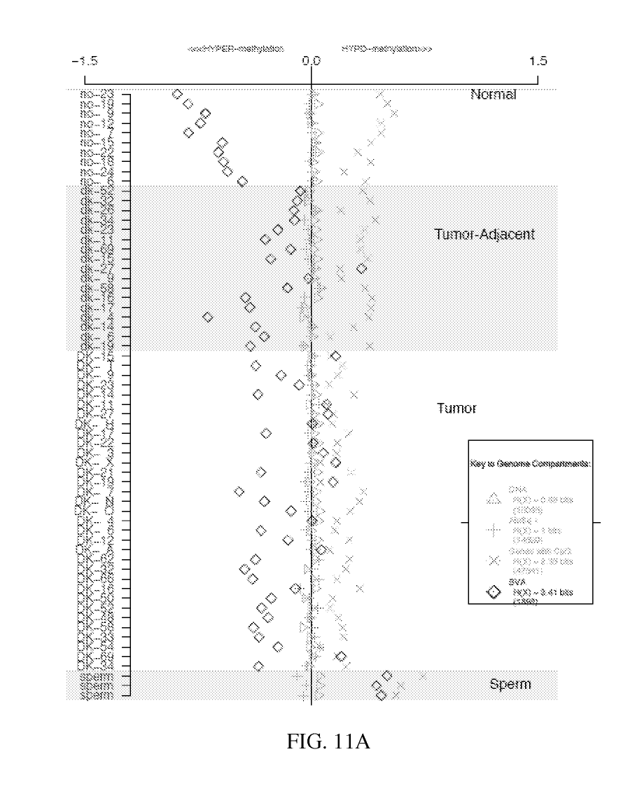

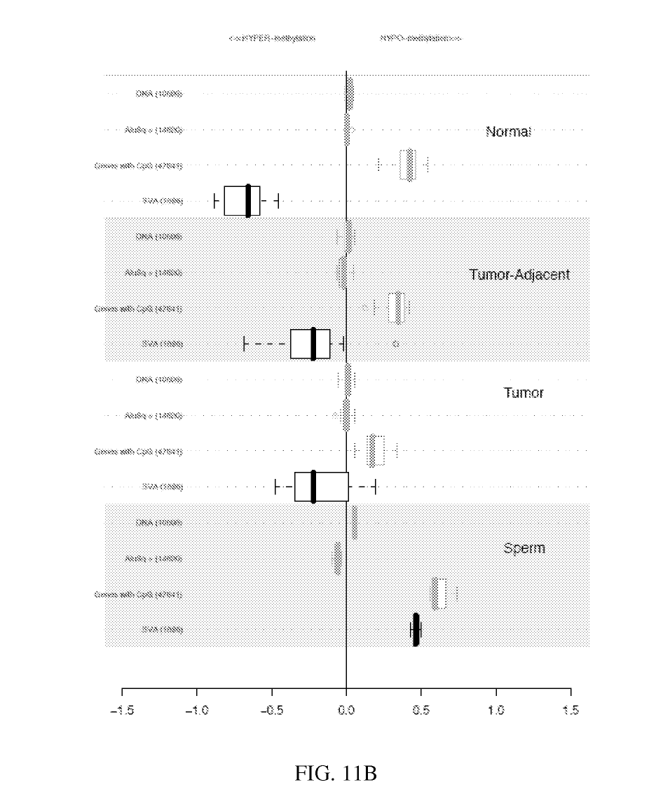

Given that each probe in the microarray is annotated with its association to the proximal genomic elements (repetitive element category, gene, miRNA) for every experiment in the library, a query is issued to retrieve a subset of probes in the vicinity of a specific element. The set of probes (from which the subset of probes are retrieved) are complementary to unique sequences in loci containing CpG islands or CpG islets the selection of which is described elsewhere herein. A set of values from the probes in the retrieved subset of probes is then averaged per experiment and plotted accordingly (note, instead of average, any other function can be applied here). This is repeated for every experiment and for every category requested. FIG. 11A is an example of this plot for 4 categories of genomic compartments. Contrast with FIG. 11B, which shows the same information in per-category view. A standard boxplot implementation included in R programming language was embedded in a custom script to generate these plots.

b. Per-Category Plots

An alternative to the plots described above are the per-category plots, devised to simplify the presentation of information especially when many categories of repetitive elements are to be plotted. For these plots, once an average of a given category of probes is calculated for all experiments, a box-and-whisker plot is then generated to summarize these values for experiment subsets: normal (top), non-tumor adjacent, tumor and sperm (bottom). FIG. 11B is an example of this plot for 4 categories of genomic compartments. Contrast with FIG. 11A, which shows the same information in per-experiment mode.

A standard boxplot implementation included in R programming language was embedded in a custom script to generate these plots.

c. On Order of Experiments

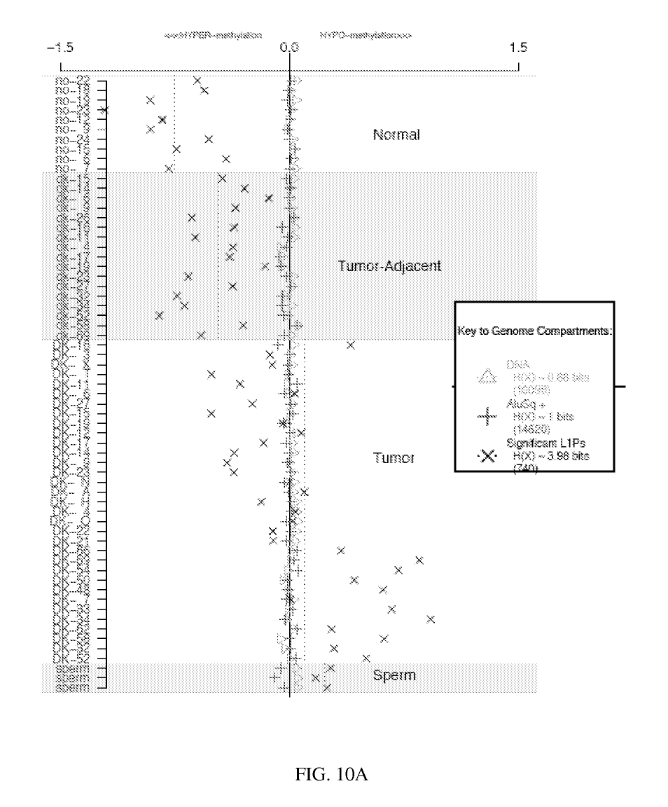

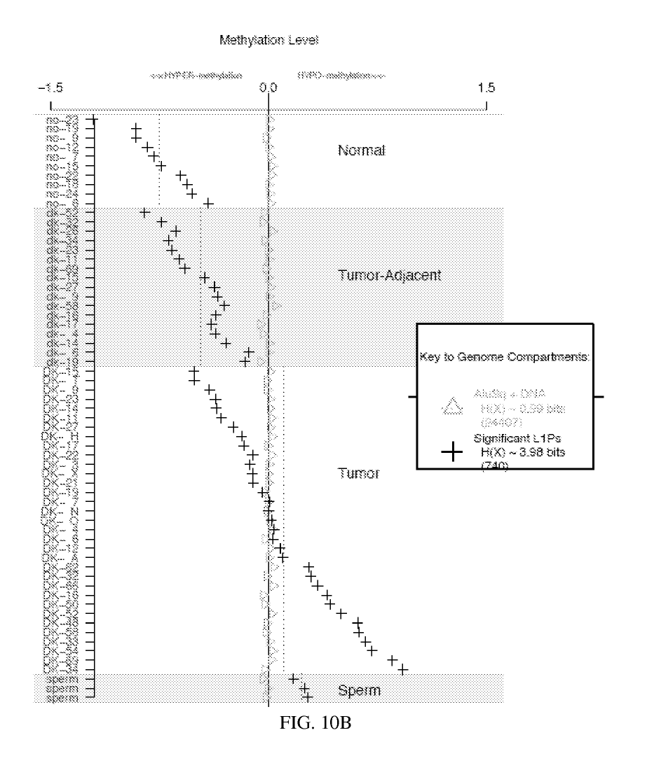

The experiments are always grouped (top to bottom) into normal, non-tumor adjacent, tumor and sperm-replicate classes. The order of experiments across all plots (unless stated otherwise) is kept constant. The order has been established based on the difference of most informative category of L1P (Table 9) versus the most stable across all experiments categories of repetitive elements: AluSq and DNA transposons. FIGS. 10A and 10B depicts the element values used.

TABLE 9
A summary of an enrichment analysis. A summary of an enrichment
analysis where a set of probes significantly differentiating tumors and non-tumor adjacent
experiments was chosen using Wilcoxon non-parametric t.test and Benjamini-Hochenberg
FDR correction to arrive at 15,587 probes. Probe categories defined by their proximity to a
specific category of repetitive element were then checked for their enrichment in significant
probes. The expected number is calculated based on the total number of probes in the array
339,314, and the total number of probes in a given category. The enrichment is then confirmed
using hyper geometric test. Table sorted based on p-values, from most significant to least significant.

| | # of probes | | | | | | |
|---|---|---|---|---|---|---|---|
| | significantly | | | | | | |
| | differentiating | | | | | | |
| | non-tumor | | | total | | | |
| | adjacents and | | | number of | | # of | # of |
| | tumors (no | expected at | | probes in | hyperGeometric | genomic | probed |
| category | pairs) | random | enrichment | category | p-val | repeats | repeats |
| L1P | 800 | 18.9 | 42 | 8,000 | 3.18E−151 | 1,589 | 8,681 |
| L1 | 2913 | 452.8 | 6 | 52,747 | 8.70E−139 | 895,230 | 64,191 |
| LINE | 3699 | 829.7 | 4 | 76,107 | 2.08E−101 | 1,354,049 | 92,711 |
| L1PA3 | 223 | 0.8 | 295 | 1,150 | 9.25E−96 | 10,281 | 1,146 |
| AluY | 3211 | 680.7 | 5 | 71,931 | 7.80E−48 | 135,761 | 72,224 |
| SINE | 7232 | 3,919.1 | 2 | 183,877 | 2.42E−39 | 1,744,457 | 245,936 |
| L1PA2 | 121 | 0.3 | 382 | 888 | 1.06E−36 | 4,666 | 874 |
| L1PA4 | 96 | 0.2 | 572 | 593 | 9.76E−36 | 11,462 | 602 |
| L1M | 1891 | 227.7 | 8 | 40,860 | 7.82E−34 | 11,466 | 50,879 |
| Alu | 6390 | 3,073.2 | 2 | 163,190 | 2.17E−28 | 1,160,276 | 205,535 |
| LTR | 1539 | 151.1 | 10 | 33,309 | 6.57E−27 | 641,720 | 40,016 |
| L1HS | 56 | 0.1 | 961 | 353 | 2.11E−21 | 1,696 | 357 |
| L1PA5 | 47 | 0.1 | 812 | 418 | 1.73E−12 | 10,904 | 428 |
| ERV1 | 619 | 24.2 | 26 | 13,292 | 7.68E−12 | 175,007 | 16,078 |
| L1MC | 449 | 12.6 | 36 | 9,506 | 9.72E−10 | 16,426 | 11,128 |
| MaLR | 663 | 29.0 | 23 | 14,865 | 1.60E−09 | 327,397 | 16,710 |
| ERVL | 287 | 4.9 | 59 | 5,763 | 9.40E−09 | 128,284 | 6,401 |
| L1P1 | 21 | 0.0 | 2,247 | 151 | 2.34E−08 | 2,860 | 149 |
| L1MA1 | 41 | 0.1 | 691 | 491 | 2.29E−07 | 3,940 | 525 |
| L1PA7 | 31 | 0.0 | 1,019 | 333 | 4.86E−07 | 12,334 | 343 |
| L1MCc | 27 | 0.0 | 1,234 | 275 | 7.76E−07 | 6,889 | 340 |
| L1ME3 | 170 | 1.7 | 103 | 3,304 | 1.16E−06 | 7,572 | 3,897 |
| MLT1A | 100 | 0.5 | 193 | 1,756 | 2.60E−06 | 8,650 | 1,929 |
| U3 | 19 | 0.0 | 1,984 | 171 | 3.46E−06 | 228 | 103 |
| L1MC3 | 55 | 0.1 | 411 | 826 | 4.84E−06 | 12,792 | 963 |
| L1MA9 | 47 | 0.1 | 506 | 671 | 5.48E−06 | 14,394 | 745 |
| L2 | 1048 | 80.3 | 13 | 26,009 | 1.03E−05 | 403,973 | 27,360 |
| LTR7 | 42 | 0.1 | 568 | 597 | 1.38E−05 | 2,349 | 610 |
| L1PA16 | 39 | 0.1 | 625 | 543 | 1.62E−05 | 13,098 | 589 |
| L1MD | 160 | 1.5 | 104 | 3,250 | 2.34E−05 | 10,847 | 3,749 |
| L1ME1 | 98 | 0.5 | 186 | 1,821 | 3.01E−05 | 26,938 | 2,131 |
| L1ME3B | 91 | 0.4 | 202 | 1,677 | 4.05E−05 | 27,528 | 1,993 |
| L1MC1 | 49 | 0.1 | 437 | 777 | 5.63E−05 | 11,868 | 864 |
| THE1D | 34 | 0.0 | 704 | 482 | 6.96E−05 | 12,381 | 512 |
| L1MA2 | 25 | 0.0 | 1,054 | 322 | 1.06E−04 | 6,911 | 347 |
| L1M4 | 108 | 0.7 | 159 | 2,131 | 1.37E−04 | 19,504 | 2,520 |
| L1PA15 | 28 | 0.0 | 884 | 384 | 1.43E−04 | 8,233 | 429 |
| L1PB | 70 | 0.3 | 268 | 1,264 | 1.43E−04 | 1,520 | 1,379 |
| AluSc | 504 | 18.0 | 28 | 12,148 | 1.88E−04 | 48,789 | 11,969 |
| ERVK | 53 | 0.1 | 372 | 911 | 2.35E−04 | 10,461 | 1,019 |
| L1P4 | 23 | 0.0 | 1,116 | 304 | 2.67E−04 | 5,156 | 328 |
| L1M4c | 29 | 0.0 | 806 | 421 | 3.00E−04 | 7,787 | 504 |
| L1MB1 | 21 | 0.0 | 1,257 | 270 | 3.05E−04 | 5,670 | 299 |
| LTR2 | 46 | 0.1 | 440 | 771 | 3.11E−04 | 847 | 861 |
| L1PA8 | 27 | 0.0 | 881 | 385 | 3.38E−04 | 7,715 | 405 |
| MIR | 1517 | 176.1 | 9 | 39,390 | 3.63E−04 | 584,181 | 40,401 |
| L3 | 170 | 1.9 | 92 | 3,702 | 4.11E−04 | 47,228 | 3,473 |
| L1MA6 | 17 | 0.0 | 1,593 | 213 | 6.72E−04 | 4,693 | 238 |
| L1MA4A | 21 | 0.0 | 1,170 | 290 | 7.97E−04 | 5,823 | 334 |
| Tigger3b | 26 | 0.0 | 875 | 388 | 8.30E−04 | 4,998 | 434 |
| HERVH | 25 | 0.0 | 917 | 370 | 8.89E−04 | 5,774 | 461 |
| LTR33A | 15 | 0.0 | 1,844 | 184 | 9.61E−04 | 3,521 | 174 |
| MLT1A1 | 23 | 0.0 | 1,019 | 333 | 9.71E−04 | 6,611 | 371 |
| L1M2 | 29 | 0.0 | 749 | 453 | 9.82E−04 | 9,352 | 524 |
| MER57A | 20 | 0.0 | 1,229 | 276 | 9.95E−04 | 2,633 | 323 |
| L1M4b | 27 | 0.0 | 816 | 416 | 1.11E−03 | 7,423 | 504 |
| L1MA4 | 42 | 0.1 | 463 | 733 | 1.15E−03 | 8,983 | 824 |
| L1MA10 | 21 | 0.0 | 1,131 | 300 | 1.23E−03 | 5,419 | 313 |
| Tigger1 | 45 | 0.1 | 422 | 805 | 1.35E−03 | 11,928 | 951 |
| LTR33 | 38 | 0.1 | 517 | 656 | 1.49E−03 | 9,302 | 668 |
| L1MCb | 11 | 0.0 | 2,759 | 123 | 1.51E−03 | 2,863 | 147 |
| MER94 | 17 | 0.0 | 1,475 | 230 | 1.61E−03 | 4,680 | 232 |
| L1PA13 | 25 | 0.0 | 872 | 389 | 1.80E−03 | 10,722 | 410 |
| MLT1E2 | 15 | 0.0 | 1,731 | 196 | 1.85E−03 | 3,960 | 213 |
| L1PB1 | 30 | 0.0 | 683 | 497 | 2.09E−03 | 11,421 | 532 |
| MER39 | 20 | 0.0 | 1,124 | 302 | 2.96E−03 | 3,125 | 339 |
| AcHobo | 56 | 0.2 | 311 | 1,092 | 3.05E−03 | 18,762 | 1,106 |
| MLT1J | 64 | 0.2 | 265 | 1,280 | 3.12E−03 | 15,375 | 1,332 |
| L1ME3A | 56 | 0.2 | 309 | 1,097 | 3.35E−03 | 16,330 | 1,269 |
| L1MD1 | 27 | 0.0 | 752 | 451 | 3.53E−03 | 6,126 | 523 |
| MER4D | 16 | 0.0 | 1,482 | 229 | 3.67E−03 | 1,275 | 259 |
| MER103 | 22 | 0.0 | 967 | 351 | 4.04E−03 | 6,812 | 348 |
| MSTD | 23 | 0.0 | 910 | 373 | 4.23E−03 | 7,421 | 412 |
| LTR12_{—} | 11 | 0.0 | 2,406 | 141 | 4.67E−03 | 664 | 136 |
| THE1C | 20 | 0.0 | 1,064 | 319 | 5.52E−03 | 9,731 | 330 |
| MLT1A0 | 51 | 0.2 | 335 | 1,014 | 6.23E−03 | 20,156 | 1,102 |
| L1PA10 | 17 | 0.0 | 1,290 | 263 | 6.59E−03 | 6,962 | 271 |
| MLT1J2 | 21 | 0.0 | 984 | 345 | 6.60E−03 | 7,867 | 331 |
| L1MD2 | 44 | 0.1 | 395 | 858 | 7.07E−03 | 12,388 | 990 |
| L1MA8 | 29 | 0.0 | 650 | 522 | 7.63E−03 | 9,992 | 576 |
| MER57A- | 9 | 0.0 | 2,900 | 117 | 9.06E−03 | 1,965 | 146 |
| int | | | | | | | |
| Mariner | 47 | 0.1 | 361 | 941 | 9.07E−03 | 16,055 | 984 |
| MER4A | 19 | 0.0 | 1,084 | 313 | 9.07E−03 | 1,084 | 366 |
| MIR3 | 213 | 3.2 | 66 | 5,136 | 1.05E−02 | 70,044 | 4,603 |
| LTR9 | 18 | 0.0 | 1,139 | 298 | 1.10E−02 | 1,919 | 314 |
| MLT2B1 | 12 | 0.0 | 1,917 | 177 | 1.11E−02 | 4,186 | 191 |
| L1PA6 | 13 | 0.0 | 1,722 | 197 | 1.13E−02 | 5,617 | 201 |
| L1MEe | 37 | 0.1 | 471 | 720 | 1.13E−02 | 12,492 | 890 |
| THE1B | 33 | 0.1 | 539 | 630 | 1.17E−02 | 22,106 | 659 |
| MLT2A2 | 8 | 0.0 | 3,263 | 104 | 1.20E−02 | 3,754 | 106 |
| HERV16 | 11 | 0.0 | 2,108 | 161 | 1.28E−02 | 4,493 | 183 |
| L1MEc | 56 | 0.2 | 288 | 1,179 | 1.35E−02 | 21,328 | 1,421 |
| L1MA3 | 25 | 0.0 | 741 | 458 | 1.41E−02 | 8,131 | 490 |
| LTR16B | 12 | 0.0 | 1,834 | 185 | 1.55E−02 | 3,456 | 183 |
| PiggyBac | 9 | 0.0 | 2,630 | 129 | 1.71E−02 | 2,068 | 138 |
| DNA | 948 | 69.9 | 14 | 25,027 | 1.84E−02 | 386,466 | 28,225 |
| CR1 | 135 | 1.3 | 106 | 3,198 | 2.00E−02 | 54,846 | 3,185 |
| MER50 | 19 | 0.0 | 998 | 340 | 2.05E−02 | 2,576 | 390 |
| MER2_type | 206 | 3.1 | 67 | 5,048 | 2.05E−02 | 81,748 | 5,863 |
| MLT1E | 30 | 0.1 | 577 | 588 | 2.06E−02 | 3,378 | 660 |
| MER2 | 287 | 6.1 | 47 | 7,202 | 2.25E−02 | 8,174 | 8,171 |
| L1P3 | 11 | 0.0 | 1,928 | 176 | 2.41E−02 | 4,518 | 184 |
| MIRm | 108 | 0.8 | 134 | 2,528 | 2.45E−02 | 32,156 | 2,288 |
| L1M1 | 25 | 0.0 | 704 | 482 | 2.49E−02 | 10,118 | 540 |
| LTR1 | 165 | 1.9 | 85 | 4,006 | 2.54E−02 | 1,396 | 4,070 |
| MER6 | 84 | 0.5 | 177 | 1,922 | 2.55E−02 | 926 | 2,123 |
| MER8 | 41 | 0.1 | 393 | 864 | 2.77E−02 | 1,884 | 891 |
| ERVL-E | 21 | 0.0 | 855 | 397 | 2.84E−02 | 9,525 | 453 |
| L1PB4 | 17 | 0.0 | 1,095 | 310 | 2.99E−02 | 6,330 | 328 |
| MER39B | 7 | 0.0 | 3,294 | 103 | 3.07E−02 | 1,166 | 113 |
| LTR67 | 17 | 0.0 | 1,091 | 311 | 3.07E−02 | 6,289 | 306 |
| FAM | 31 | 0.1 | 537 | 632 | 3.08E−02 | 4,424 | 525 |
| MLT1I | 26 | 0.0 | 655 | 518 | 3.27E−02 | 11,191 | 533 |
| MER4A1 | 13 | 0.0 | 1,495 | 227 | 3.34E−02 | 2,150 | 267 |
| L1PA14 | 9 | 0.0 | 2,356 | 144 | 3.35E−02 | 3,561 | 160 |
| MLT2B3 | 8 | 0.0 | 2,693 | 126 | 3.63E−02 | 2,966 | 126 |
| MLT1F | 33 | 0.1 | 490 | 692 | 3.84E−02 | 4,498 | 769 |
| L1MC5 | 67 | 0.3 | 220 | 1,544 | 4.42E−02 | 20,412 | 1,780 |
| AluYg | 19 | 0.0 | 915 | 371 | 4.47E−02 | 111 | 363 |
| L1MA5 | 20 | 0.0 | 857 | 396 | 4.67E−02 | 4,086 | 435 |
| MIRb | 736 | 42.4 | 17 | 19,543 | 4.77E−02 | 278,083 | 19,026 |
| MADE2 | 9 | 0.0 | 2,203 | 154 | 4.91E−02 | 2,609 | 139 |
| LTR49 | 9 | 0.0 | 2,203 | 154 | 4.91E−02 | 1,406 | 182 |

d. Shannon Information Value

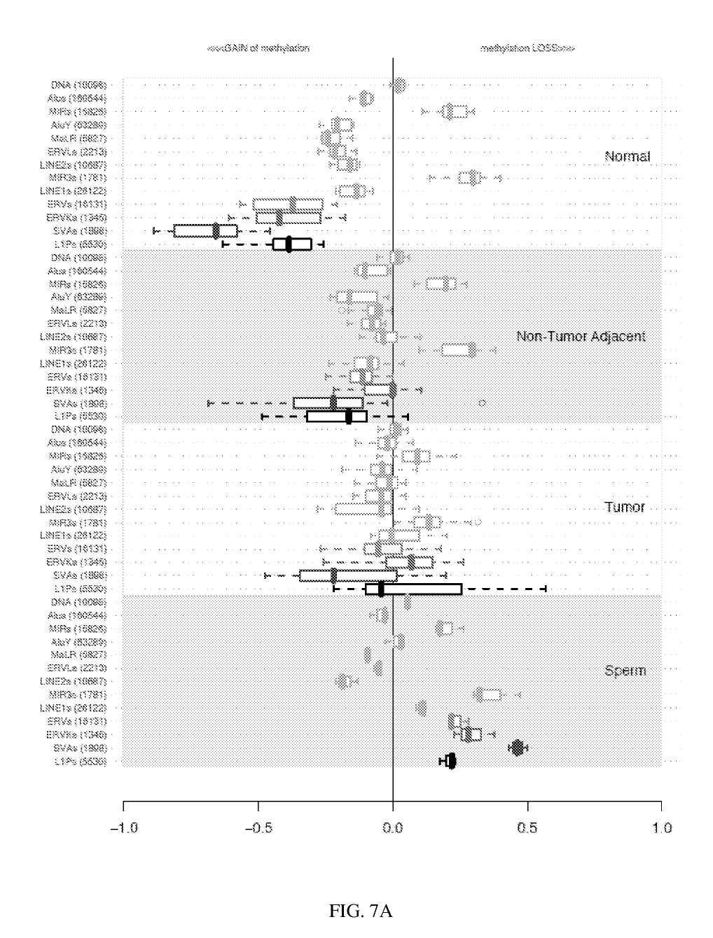

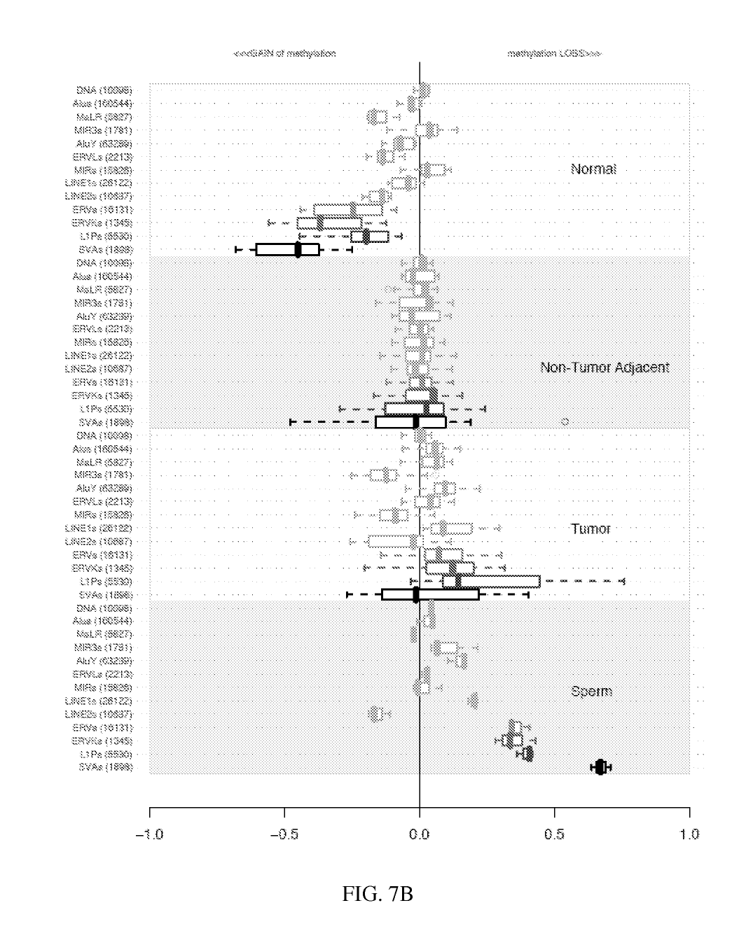

The order of categories in the legend of per-experiment plot and the category-list of per-category plots is not accidental. The categories are ordered based on the extent of their variation using Shannon information content metric (available at cm.bell-labs.com/cm/ms/what/shannonday/shannon1948.pdf). Only Normal and Tumor experiments were used to establish the order of categories. Specifically, once the per-category values (average methylation for FIGS. 7A and 7B) across normal (10 values per category) and tumor (33 values per category) experiments are calculated, a Shannon information measure function is applied to the distribution of 43 values.

The Shannon Information measure is a foundation of modern Information theory and was devised to estimate the minimum number of bits needed to encode sentence or a string of characters of text, if one wanted to transmit such string digitally. The information measure takes into consideration the frequency of the symbols. As a result, a string made up of the same symbol would require a very simple encoding using one bit of information, whereas a string made up of all the letters in the alphabet would need considerably more bits to represent all the letters unambiguously.

Analogously, the 43 values can be considered as the individual letters of Shannon's string. Shannon's entropy measures how dissimilar the 43 values are from each other. The more dissimilar, the more information is in the set.

The categories are listed from lowest information content (top) to the highest information content (bottom). The most informative categories are highlighted. A custom R script was used to generate the plots and calculate the information content.

3. Results and Discussion

i. Methylation Patterns of Major Classes and Families of DNA Repeats

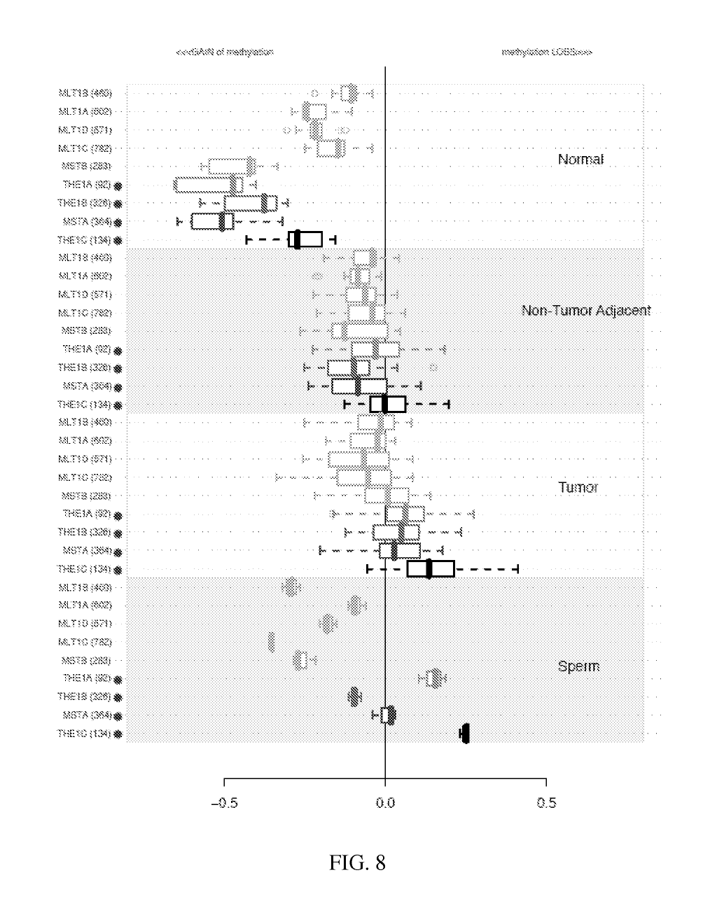

The DNA methylation profiles of 33 tumors and 17 non-tumor adjacent tissue samples obtained from patients with head and neck squamous carcinoma (HNSCC) were analyzed. DNA methylation profiles were also generated from the buccal epithelia of 10 normal individuals, which served as controls. In addition, an analysis of sperm DNA was performed in technical triplicate to assess the reproducibility of the microarray results. An average methylation value for selected subsets of “genomic probe compartments” was calculated. An exemplary profile of average methylation for two extremely different genomic probe compartments can be found in FIG. 11A, which shows DNA methylation values for all SVA elements as well as methylation values for all genes for each of the tissue sample experiments. Then all average metrics per tissue class were combined, generating a distribution of averages, shown in FIG. 11B. FIGS. 7 and 8 all use a box and whiskers distribution plot to display DNA methylation trends for different classes of repetitive elements.

FIG. 7A depicts the distribution of all averages of DNA methylation values across all experiments for each of the major repetitive element families (as summarized by Mandal and Kazazian, 2008). Two primate-specific families of repeats, AluY (Alu) and LIP (LINE-1), were also included and will be discussed at length in the ‘methylation of Alu elements’ and ‘methylation of LINE-1 elements’ sections, respectively. Gain of methylation is represented by values on the negative scale of the x-axis, and loss of methylation by values on the positive scale, towards the right side of the plots. Each subsection of the plot features the same families of repetitive elements in the same order for normal, non-tumoral adjacent, tumor and replicated sperm experiments. The order of repetitive families was established based on the information content (Shannon entropy metrics) of the methylation values among normal and tumor experiments (see Materials/Methods section ‘Plotting the data’). The 5 most “informative” families of repeats are plotted towards the bottom of the plot's subsection and highlighted. The most informative is a primate-specific subset of probes for a subcategory of LINE-1. Annotation provided by RepBase (Jurka, 1998) allowed for the investigation of subclasses of repetitive elements in greater detail. The section on ‘methylation of LINE-1 elements’ expands on the analysis of L1P elements delving into finer subcategories of these repetitive elements.

An alternative way of examining the DNA methylation data is to compare methylation levels that have been normalized with respect to the values of the non-tumor adjacent tissue, as shown in FIG. 7B. This adjacent-tissue-normalized plot makes it clear that the average methylation levels of several classes of repetitive elements in tumors are not dramatically different from those in the non-tumor adjacent material, while the normal buccal mucosa shows much higher methylation, especially for SVA, ERV, ERVK, and UP elements. Remarkably, it appears that the cumulative differences of loss of methylation for certain classes of elements such as SVA are as large between the normal and the non-tumoral adjacent tissue as they are between tumors and normal tissue samples. Previous reports in the head and neck cancer literature (Smith et al., 2007) indicate that normal buccal epithelium of individuals exposed to cigarette smoke have abnormally reduced levels of global methylation of LINE-1 elements, as determined by bisulfite sequencing of LINE-1 PCR amplicons. Even lower methylation levels were reported by these authors in cancer tissues, with advanced tumors (stages II and IV) showing the lowest methylation levels. It has also been reported that in colon cancer patients, non-tumor adjacent regions of colonic mucosa show significant loss of methylation of LINE-1 retrotransposons as compared to colonic mucosa of normal individuals (Suter et al., 2004), while colon cancer tissue shows even more pronounced loss of methylation. FIGS. 7A and 7B indicate that various subcategories of larger repetitive element families contribute disproportionately to the DNA methylation changes of their parent category. The following sections adhere to the plot style in FIG. 7A, which most accurately represents the raw data generated by the microarray analysis, and also shows the best fit to the DNA methylation values obtained independently by bisulfite sequencing of PCR products of specific probed loci. For example, in FIG. 7A it is clear that MIR elements (representing DNA transposons), are relatively unmethylated in normal mucosa, in non-tumor adjacent tissue, and in sperm. Notably, only in tumor tissue is a gain of methylation of these DNA transposons observed. These interesting relative methylation relationships are more difficult to observe, and less indicative of the actual methylation levels, as displayed in FIG. 7B due to the effects of normalization.

The following sections will focus on exploring in greater detail the dynamics of methylation patterns among various sequence sub-compartments of a given class of repetitive elements. To facilitate navigation through the perhaps unfamiliar nomenclature that identifies the various subclasses of elements, four tables are included that list the different subclasses within a class, in chronological order of estimated evolutionary age. These tables (Tables 10A through 10D) can be found in the supplementary materials. A table for the ERV retroelements is not included because the evolutionary ages and phylogenetic relationships of these elements are still a subject of investigation and revised annotation. To facilitate additional exploration of the data sets, the methylation levels can be grouped by subclass of repetitive element, rather than by tissue type (as in FIGS. 7 and 8).

TABLE 10A
A compilation of observations about ages of lineages within
families of repetitive elements.

| | | Estimated age of | |
|---|---|---|---|
| | Element | appearance in the | |
| Element Family | subfamily | genome (MYA) | references |
| MaLR | MLT1d | 100 | FIG. 4 in Smit, 1993 |
| MaLR | MLT1c | 85 | FIG. 4 in Smit, 1993 |
| MaLR | MLT1b | 80 | FIG. 4 in Smit, 1993 |
| MaLR | MLT1a | 77 | FIG. 4 in Smit, 1993 |
| MaLR | MSTb | 75 | FIG. 4 in Smit, 1993 |
| MaLR | MSTa | 60 | FIG. 4 in Smit, 1993 |
| MaLR | THE1c | 55 | FIG. 4 in Smit, 1993 |
| MaLR | THE1b | 50 | FIG. 4 in Smit, 1993 |
| MaLR | THE1a | 45 | FIG. 4 in Smit, 1993 |

TABLE 10B
A compilation of observations about ages of lineages within
families of repetitive elements.

| | | Estimated age of | |
|---|---|---|---|
| | Element | appearance in the | |
| Element Family | subfamily | genome (MYA) | references |
| Alu | FLA | 112 | Table 3 in Kapitonov |
| | | | and Jurka, 1996 |
| Alu | Jo | 44, 81 | Table 3 in Kapitonov |
| | | | and Jurka, 1996, Table |
| | | | S1 in Pace and |
| | | | Feschotte, 2007 |
| Alu | Jb | 42, 81 | Table 3 in Kapitonov |
| | | | and Jurka, 1996, Table |
| | | | S1 in Pace and |
| | | | Feschotte, 2007 |
| Alu | S | 48 | Table 3 in Kapitonov |
| | | | and Jurka, 1996 |
| Alu | Sx | 28, 37, 44, 55 | Table 3 in Kapitonov |
| | | | and Jurka, 1996, FIG. |
| | | | 2 in Batzer and |
| | | | Deininger, 2002, Table |
| | | | S1 in Pace and |
| | | | Feschotte, 2007, Table |
| | | | 2 in Xing et al. 2004 |
| Alu | Sg | 23, 31, 34, 40 | Table 3 in Kapitonov |
| | | | and Jurka, 1996, FIG. |
| | | | 2 in Batzer and |
| | | | Deininger, 2002, Table |
| | | | S1 in Pace and |
| | | | Feschotte, 2007, Table |
| | | | 2 in Xing et al. 2004 |
| Alu | Sq | 25, 39, 44 | Table 3 in Kapitonov |
| | | | and Jurka, 1996, Table |
| | | | S1 in Pace and |
| | | | Feschotte, 2007, Table |
| | | | 2 in Xing et al. 2004 |
| Alu | Sp | 28, 37 | Table 3 in Kapitonov |
| | | | and Jurka, 1996, Table |
| | | | 2 in Xing et al. 2004 |
| Alu | Sc | 22, 30, 35 | Table 3 in Kapitonov |
| | | | and Jurka, 1996, Table |
| | | | S1 in Pace and |
| | | | Feschotte, 2007, Table |
| | | | 2 in Xing et al. 2004 |
| Alu | Sb | 19 | Table 3 in Kapitonov |
| | | | and Jurka, 1996 |
| Alu | Y | 16, 35 | FIG. 2 in Batzer and |
| | | | Deininger, 2002, Table |
| | | | S1 in Pace and |
| | | | Feschotte, 2007 |
| Alu | Yd | 15 | Table 2 in Xing et al. |
| | | | 2004 |
| Alu | Ye5 | 12 | Table 2 in Xing et al. |
| | | | 2004 |
| Alu | Yb8 | 11 | FIG. 2 in Batzer and |
| | | | Deininger, 2002 |
| Alu | Ya5 | 2, 11 | FIG. 2 in Batzer and |
| | | | Deininger, 2002, Table |
| | | | 2 in Xing et al. 2004 |
| Alu | Yc1 | 2, 5 | FIG. 2 in Batzer and |
| | | | Deininger, 2002, Table |
| | | | 2 in Xing et al. 2004 |
| Alu | Ya5a2 | 5 | FIG. 2 in Batzer and |
| | | | Deininger, 2002 |
| Alu | Yg6 | 3 | Table 2 in Xing et al. |
| | | | 2004 |
| Alu | Yb9 | 2, 5 | FIG. 2 in Batzer and |
| | | | Deininger, 2002, Table |
| | | | 2 in Xing et al. 2004 |
| Alu | Ya8 | 1 | Table 2 in Xing et al. |
| | | | 2004 |

TABLE 10C
A compilation of observations about ages of lineages within
families of repetitive elements.

| | | Estimated age of | |
|---|---|---|---|
| | Element | appearance in the | |
| Element Family | subfamily | genome (MYA) | references |
| SVA | A | 10-17 | Table 2 in Wang et al. |
| | | | 2005 |
| SVA | B | 11-12 | Table 2 in Wang et al. |
| | | | 2005 |
| SVA | C | 10-11 | Table 2 in Wang et al. |
| | | | 2005 |
| SVA | D | 9.4-9.5 | Table 2 in Wang et al. |
| | | | 2005 |
| SVA | E | 2.4-4.5 | Table 2 in Wang et al. |
| | | | 2005 |
| SVA | F | 2.7-3.6 | Table 2 in Wang et al. |
| | | | 2005 |

TABLE 10D
A compilation of observations about ages of lineages within
families of repetitive elements.

| | | Estimated age of | |
|---|---|---|---|
| | | appearance | |
| Element | Element | in the genome | |
| Family | subfamily | (MYA) | references |
| L1PA | L1PA1 | 3.1 | Table 1 in Khan et al. 2006 |
| L1PA | L1PA2 | 7.6 | Table 1 in Khan et al. 2006 |
| L1PA | L1PA3 | 12.5 | Table 1 in Khan et al. 2006 |
| L1PA | L1PA4 | 18.0 | Table 1 in Khan et al. 2006 |
| L1PA | L1PA5 | 20.4 | Table 1 in Khan et al. 2006 |
| L1PA | L1PA6 | 26.8 | Table 1 in Khan et al. 2006 |
| L1PA | L1PA7 | 31.4 | Table 1 in Khan et al. 2006 |
| L1PA | L1PA8 | 40.9 | Table 1 in Khan et al. 2006 |
| L1PA | L1PA8A | 41.7 | Table 1 in Khan et al. 2006 |
| L1PA | L1PA10 | 46.4 | Table 1 in Khan et al. 2006 |
| L1PA | L1PA11 | 53.3 | Table 1 in Khan et al. 2006 |
| L1PA | L1PA13B | 59.8 | Table 1 in Khan et al. 2006 |
| L1PA | L1PA12 | 59.7 | Table 1 in Khan et al. 2006 |
| L1PA | L1PA13A | 59.9 | Table 1 in Khan et al. 2006 |
| L1PA | L1PA14 | 60.6 | Table 1 in Khan et al. 2006 |
| L1PA | L1PA15 | 70.5 | Table 1 in Khan et al. 2006 |
| L1PA | L1PA16 | 79.7 | Table 1 in Khan et al. 2006 |
| L1PA | L1PA17 | 101.1 | Table 1 in Khan et al. 2006 |
| L1PB | L1PB1 | 46.7 | Table 1 in Khan et al. 2006 |
| L1PB | L1PB2 | 58.2 | Table 1 in Khan et al. 2006 |
| L1PB | L1PB3 | 73.5 | Table 1 in Khan et al. 2006 |
| L1PB | L1PB4 | 96.0 | Table 1 in Khan et al. 2006 |
| L1MA | L1MA1 | 61.6 | Table 1 in Khan et al. 2006 |
| L1MA | L1MA2 | 65.8 | Table 1 in Khan et al. 2006 |
| L1MA | L1MA3 | 68.1 | Table 1 in Khan et al. 2006 |
| L1MA | L1MA4 | 102.3 | Table 1 in Khan et al. 2006 |
| L1MA | L1MA5 | 98.6 | Table 1 in Khan et al. 2006 |

ii. Methylation of MaLR Elements

Since observing that AluY and UP, two primate-specific subfamilies of repeats scored higher using the information content statistic than their respective (and all-inclusive) parents, a relatively well-annotated family tree of “mammalian apparent LTR retrotransposons” (MaLR) was investigated (Smit, 1993). An analysis involving specific subsets of MaLR is shown in FIG. 8. Here, again, the youngest members of the MaLR elements, THE1A, THE1B, THE1C, MSTA, and MSTB which only exist in simians and humans, are methylated in normal tissue but show marked loss of methylation in most tumors. Older MaLR subfamilies are less methylated in normal tissue, and show less striking loss of methylation in tumors. It is intriguing that among the younger families, THE1A and THE1B retroelements, which were observed to be demethylated in most non-tumoral adjacent tissue as well as tumors, have been identified as key sequence attributes of human recombination hotspots (Myers et al., 2005, 2008). The possible contribution of these subfamilies of demethylated MaLR elements to recombinational events in tumors is an interesting subject for future investigation.

iii. Methylation of Alu Elements

Alu elements are the most abundant class of repetitive elements in the human genome with over one million copies and spanning over 30 lineages. The most detailed published analysis of Alu DNA methylation in normal cells and cancer cells was reported by Rodriguez et al. (2008). These authors targeted unmethylated SmaI sites within Alu sequences, and found that normal colon epithelial cells contain a subpopulation of undermethylated Alus, while in tumor cells the number of unmethylated Alu sequences is doubled. They also reported an increased methylation of the younger Alu subfamilies. The microarray-based analysis includes only those Alu lineages for which more than 200 unique locations were probed. As observed for other classes of elements, the younger elements (AluY) are more highly methylated in normal adult tissues, yet are suffering a greater loss of DNA methylation in many tumors. Interestingly, the oldest Alu elements remain methylated in sperm, while the younger ones show loss of methylation in this tissue. The most informative among normal and tumor tissues lineage of Alu's is AluYb. Coincidentally, it is also the most active of all Alu lineages and found primarily in human genomes (Jurka, 1993; Carter et al., 2004). AluYg, the next most informative lineage remains relatively unknown. Among other, less informative lineages, the middle-age AluS families lose methylation in tumor tissue, while the members of the oldest, AluJ lineages remain methylated at an intermediate level, and constant in all 4 tissue types.

iv. Methylation of ERV Elements

Endogenous Retrovirus (ERV) Families are a heterogeneous group of sequences with over 60 lineages according to RepBase (Jurka, 1998). There are reports of ERV sequences being involved in extensive chromosomal rearrangement during the last 30 million years in primate evolution (Romano et al., 2006). Per-lineage analysis pertaining to the methylation pattern of ERV was assessed. Similarly to MaLR and Alu discussed above, Human Endogeneous Retrovirus (HERV) families appear heavily methylated in the normal tissues. The gradual loss of methylation is apparent for HERVH and HERV17 families. To an extent, the methylation levels of HERVE and KERVK also vary among normal, tumors and non-tumoral adjacent tissues. So far, for MaLR, Alu and ERV families of ancient repetitive elements, predating the mammalian radiation, the microarray DNA methylation analysis indicates that young, primate specific lineages appear more susceptible to de-methylation in disease than other, older lineages.

v. Methylation of SVA Elements

A similar analysis was performed for SVA elements, which have been extensively mobilized in the human genome after the divergence of hominids from chimpanzees (Xing et al., 2007; Wang et al., 2007; Macfarlane and Simmonds, 2004). SVA elements consist of a combination of sequences derived from other retroelements (Babushok and Kazazian, 2007) and are known to be non-autonomous, depending on LINE-1 elements for mobilization. Wang et al. (2005) have estimated the evolutionary age of different subfamilies of SVA elements, named SVA-A through SVA-F. This analysis reveals that the youngest SVA subfamilies show an unusual relationship between evolutionary age and the level of dysregulation. SVA-F elements, which are human specific, and only 3 MY old, are significantly less methylated than other, older subfamilies, and their methylation level does not change much in different samples, with the exception of sperm, where these elements show loss of methylation. On the other hand, the SVA-A elements, which are the oldest SVA subfamily (16.81 MY), are strongly methylated in normal oral tissues, but their loss of methylation is strikingly variable among different samples, with tumors showing the greatest level of variation. Thus, the magnitude and trends of DNA methylation changes for the youngest SVA elements seems to diverge from the patterns observed for AluY, MaLR, and ERV elements. The dramatic DNA methylation dysregulation affecting most SVA subfamilies in non-tumoral adjacent tissue is particularly striking.

vi. Methylation of LINE-1 Elements

Lineages of the LINE-1 family were investigated. Categories which could be probed in at least 100 unique genomic loci. Comparing the values across the four classes of experiments, it is apparent that younger, primate specific classes of LINE-1 elements (LINE-1PA3 (L1PA3) and LINE-1PA4 (L1PA4) and LINE-1PA5 (L1PA5), none of which exist in the baboon or marmoset) are more strongly methylated in normal tissue, and suffer more dramatic losses in DNA methylation in tumors and sperm. However, similarly to the observations for SVA subfamilies, the newest LINE-1 families that are strictly human specific (L1PA2, L1HS, Full Length active LINE-1 (Penzkofer et al., 2005)) are not as highly methylated in normal tissue, and not as dramatically demethylated in tumorigenesis as the longer-established lineages, the primate-specific LINE-1PA subfamilies.

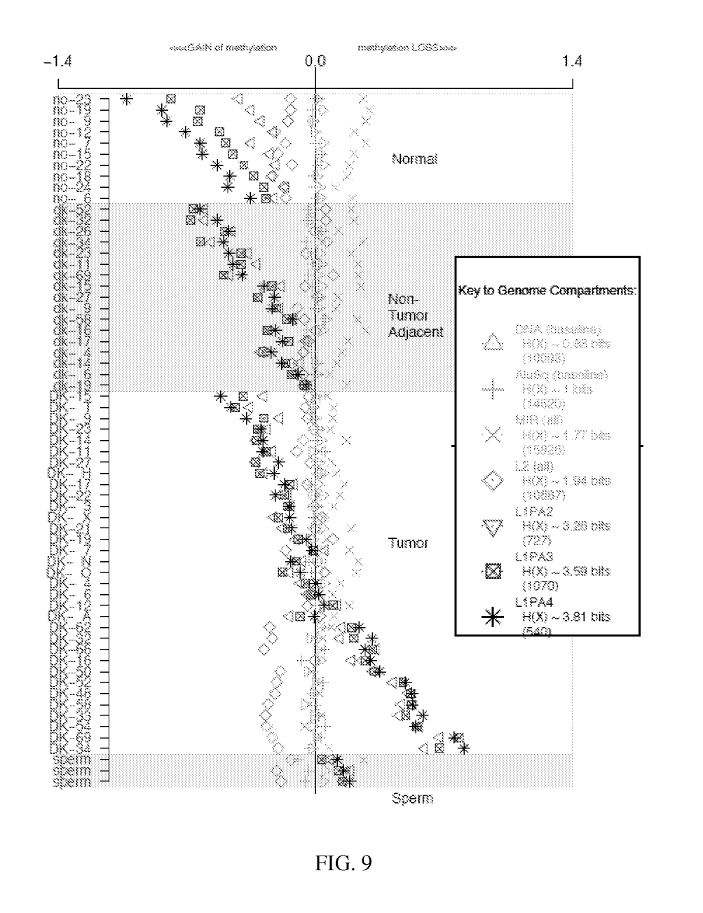

It is relevant to explore potential correlations or anti-correlations among the DNA methylation metrics within individual experiments. With this question in mind, FIG. 9 reports methylation levels in individual experiments for the family of MIR repeats, as well as the family of L2 repeats, as compared to L1PA3 and L1PA4 methylation levels. The data illustrates completely distinct and sometimes opposing trends in their levels of methylation, demonstrating that the observed metrics for the L1PA methylation levels are not due to normalization artifacts. In most cases the youngest members of each retrotransposon family are strongly methylated in normal buccal tissues, as shown by their negative values for all 10 samples from healthy adults. In tumors, the corresponding retroelement families shift dramatically to a relatively unmethylated state, as shown by the predominantly positive values. In the adjacent non-tumor tissue, the methylation level is variable, reflecting different degrees of epigenetic dysregulation in these tissues among different patients. These results underscore the fact that the methylation level of different families can be regulated (and dysregulated) independently in different tissues. Interestingly, the present data also shows that for any member of a retroelement subfamily, the methylation level in tissues from different patients can vary within certain bounds depending on the genomic sequence context, while in the sperm experiment, which represents a single individual, the methylation levels for any given family converge to a distinct and strikingly narrow range of values, characteristic of each repeat family.

vii. Analysis of Relative CpG Content Among Different Repetitive Element Subfamilies

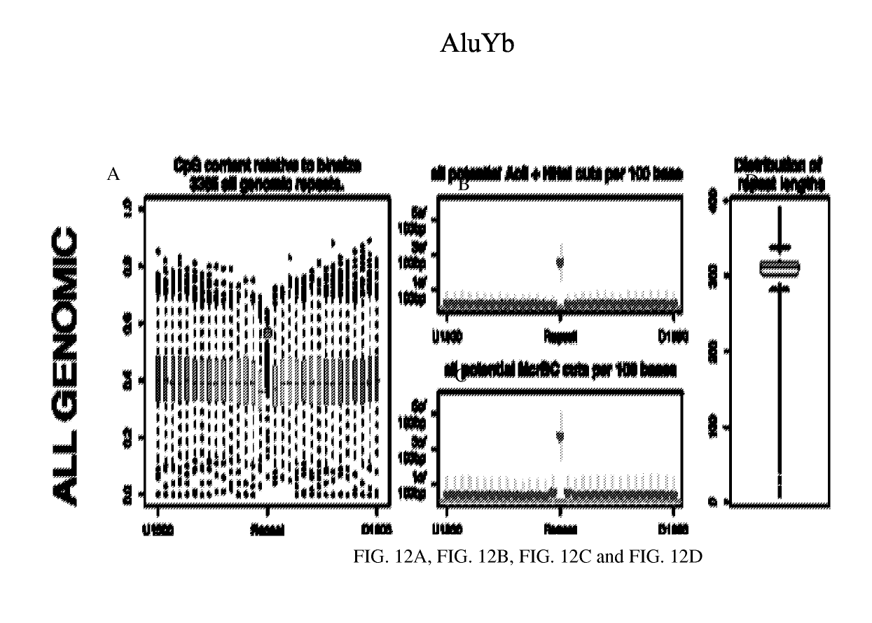

The formal possibility that some of the differences in DNA methylation levels could be influenced by the CpG content of the DNA sequences being probed was explored. This analysis involves analyzing the count of CpG residues in the repeats and the immediately surrounding sequences, as shown for a single repetitive element family in FIG. 12. All sequences annotated by repeat masker as belonging to AluYb were filtered to output only those for which there is a probe on the microarray. From all these sequences, the ones that are shorter than the median of all sequences were further excluded to focus the subsequent analysis on longer (possibly full length) elements. The alignment of 60 of those sequences selected at random was then created using clustalw2 (1.0.11) program and standard parameters. The output of clustalw2 was subsequently parsed using a series of custom python and R scripts to visualize the alignment. For example, an analysis of the CpG content of all classes of MaLR elements shows that for those elements that were probed, the CpG content, as well as the frequency of endonuclease recognition sites is not noticeably different. MLT1C elements, which show much lower methylation changes relative to MSTA elements, have almost identical metrics of CpG count and endonuclease sites. In the case of SVA elements, the analysis shows that the CpG content, as well as the frequency of endonuclease recognition sites is noticeably higher for the SVA-F elements than for the SVA-B elements that were probed. Yet, the SVA-B elements, in spite of their somewhat lower frequency of potential endonuclease cutting sites, show more dramatic differences in methylation between normal samples and tumor samples relative to the SVA-F elements. A comparison of the CpG content of L1PA17 elements shows a higher content of CpGs and endonuclease sites within a +/−400 base window of the probes, as compared to L1PA4 elements, which show lower values for both metrics. Yet, it is the L1PA4 elements that show the greater changes in DNA methylation. The L1HS (human-specific) subfamily shows a somewhat higher frequency of endonuclease sites compared to the L1PA3 subfamily, and yet the L1HS methylation levels change to a lower degree in different tissues. Analysis of the Alu elements show that the AluY subfamilies have a higher content of CpG residues and endonuclease sites compared to the relatively older AluJ and AluS subfamilies. While these differences could partially contribute to the observed smaller changes in DNA methylation observed for the older elements, the differences cannot account for all observations in DNA methylation changes. It is important to note that, using different methodology, Rodriguez et al. (2008) obtained evidence suggesting that in normal colonic epithelia the older members of the Alu family are less methylated than the younger members. In this analysis, the related AluYd8 and AluYb9 subfamilies have almost identical metrics for CpG content and endonuclease sites, and yet show marked differences in their DNA methylation changes among normal tissues, tumors, and sperm. Taken together, these observations argue against differences in the content of CpG dinucleotides as a trivial explanation for the observed differences in DNA methylation levels among different families of repetitive elements.

viii. Properties of Probes Capable of Best Distinguishing Non-Tumor Adjacent Tissue from Tumor Tissue

The foregoing analysis does not help to identify events that could be tumor-specific. To address this issue, each individual probe associated with a repetitive element was ranked on the basis of its ability to differentiate tumors from non-tumoral adjacent tissue using a Wilcoxon test. A statistical analysis involving those probes that displayed altered methylation was performed by calculating the probe values (ratios) in tumor samples, and the likelihood of random methylation changes as a function of the total number of probes belonging to any one family of repeats. The probes were ranked based on the P-values generated by a hypergeometric t-test, as shown in Table 9. The entries with the most significant P-values include members of the LINE-1P, AluY, LTR, and SVA families of interspersed repeats. Among the primate-specific L1 elements, the L1PA3, L1PA2, and L1PA4 are among the most highly enriched. Among the LTR elements, the LTR7, LTR33, and HERV elements are high on the list. AluY represents the youngest family of Alu elements, and they rank much higher than older Alu elements. The HERV and SVA elements are among the few retrotransposon families known to have been extensively mobilized in the human genome after the divergence of hominids from chimpanzees (Xing et al., 2007; Wang et al., 2005; Macfarlane and Simmonds, 2004).

The data in Tables 11A and 11B summarizes salient properties of the subset of LINE-1 elements that were identified using the Wilcoxon test, as the best DNA methylation probe variables for distinguishing tumors from non-paired non-tumoral adjacent tissue. In Table 11A, the column corresponding to relative enrichment of a set of elements shows that the highest value (4.757) corresponds to a subset of the L1PA4 subfamily. Members of the L1PA3 subfamily are also highly enriched among the most significant probes. The column specifying the median length of the elements shows that for L1PA5 and L1PA6 there is a noticeable increase in the length of the elements corresponding to the most significant probes (almost a 2-fold increase relative to all probed elements, in the case of L1PA6). A longer length could be associated with a higher likelihood of having an intact L1 promoter, as well as a higher probability of generating a full-length LINE-1 RNA transcriptional product. The table also shows enrichment of probes mapping to full-length L1 elements (FLI-L1) and ORF2-competent L1 elements (ORF2-L1, Jurka, 1998; Penzkofer, et al., 2005). L1PA4 elements, which are the most highly enriched among the significant probes, are unlikely to code for functional ORF2 proteins, and thus unlikely to generate reverse transcriptase. This observation indicates that possible positive selection in tumors for long L1 elements among the most significant probes is not operating at the level of conservation of ORF2 protein-coding function.

TABLE 11A
Enrichment of significant probes in all probes associated with young
L1P lineages. Highlighted in bold are the primate L1P lineages that appear in post-baboon
species. The 15,587 probes are the most significant probes characterized in the Table 9.
Enrichment is calculated based on all 339,314 probes in the microarray.
Hypergeometric test score is recorded as well. The two highest enrichment
values and two highest p-values are highlighted in bold.

| | TOTAL | | ALL | | | | | | | |
|---|---|---|---|---|---|---|---|---|---|---|
| | GENOMIC | | PROBED | | 15,587 Most Significant Probes | | | | | |
| | | median | | median | | | Obs/ | Hyper-geo | median | Length |
| Family | N | length | N | length | Exp | Obs | exp | pval | length | Selection |
| FLI-L1 | 145 | 8,047 | 87 | 6,029 | 4 | 9 | 2.252 | 6.61E−03 | 6,028 | (1) |
| ORF2-L1 | 103 | 8,047 | 91 | 6,026 | 4 | 13 | 3.110 | 7.00E−05 | 6,026 | 0 |
| L1HS | 1696 | 987 | 294 | 6,025 | 14 | 43 | 3.184 | 7.25E−12 | 6,026 | 2 |
| L1PA2 | 4666 | 771 | 727 | 6,024 | 33 | 115 | 3.444 | 3.56E−31 | 6,023 | (1) |
| L1PA3 | 10281 | 952 | 1070 | 6,031 | 49 | 226 | 4.598 | 2.19E−83 | 6,031 | 1 |
| L1PA4 | 11462 | 713 | 540 | 6,130 | 25 | 118 | 4.757 | 2.80E−46 | 6,132 | 2 |
| L1PA5 | 10904 | 623 | 328 | 5,659 | 15 | 46 | 3.053 | 6.91E−12 | 6,115 | 457 |
| L1PA6 | 5617 | 888 | 169 | 2,600 | 8 | 13 | 1.675 | 2.46E−02 | 4,076 | 1,476 |
| L1PA7 | 12334 | 768 | 245 | 1,018 | 11 | 23 | 2.044 | 4.46E−04 | 469 | (549) |
| L1PA8 | 7715 | 394 | 159 | 328 | 7 | 20 | 2.738 | 1.44E−05 | 275 | (54) |
| L1PA8A | 2210 | 707 | 72 | 677 | 3 | 8 | 2.419 | 5.65E−03 | 499 | (178) |
| LIPA10 | 6962 | 474 | 146 | 676 | 7 | 8 | 1.193 | 2.30E−01 | 1,217 | 542 |
| L1PA11 | 3886 | 511 | 76 | 601 | 3 | 5 | 1.432 | 1.36E−01 | 1,278 | 678 |
| L1PA12 | 2320 | 679 | 59 | 644 | 3 | 3 | 1.107 | 2.87E−01 | 372 | (272) |
| L1PA13 | 10722 | 457 | 201 | 503 | 9 | 13 | 1.408 | 8.12E−02 | 532 | 29 |
| L1PA14 | 3561 | 558 | 66 | 557 | 3 | 7 | 2.309 | 1.06E−02 | 337 | (220) |
| L1PA15 | 8233 | 547 | 173 | 502 | 8 | 16 | 2.013 | 2.73E−03 | 450 | (52) |
| L1PA15-16 | 1201 | 727 | 32 | 331 | 1 | 3 | 2.041 | 5.75E−02 | 224 | (107) |
| L1PA16 | 13098 | 482 | 247 | 489 | 11 | 22 | 1.939 | 1.15E−03 | 488 | (1) |
| L1PA17 | 4722 | 368 | 114 | 368 | 5 | 4 | 0.764 | 6.06E−01 | 560 | 192 |

TABLE 11B
A continuation of the table from 11A showing an increase of L1HS
promoter homology in L1P members with significantly altered methylation patterns. For
selected families of L1PAs discussed in Table 11A the issue of whether the statistically
significant members (Table 9) of L1 are more homologous to a promoter region of an active
i.e. intact and relatively young, full length Line1 element were addressed. A consensus
promoter region was obtained from one of the L1HS characterized by L1Base as full lentgth
and active. To generate this table, the alignment of the 700 bases long promoter region was
performed against all members of each lineages and against subset of significant members of
each of the lineages. Software BLAT and parameters designated to result in 80% homology (-
minIdentity = 80 -tileSize = 10) were used. The count of alignments per lineage was recorded in
the table. The “%” columns show the percentage of all elements in the group (either all
repeats or subset of significant repeats only) for which a BLAT alignment was found. Note
that the percentage does not reflect on sequence conservation, but is a mere statement that an
alignment using BLAT and the specified parameters is possible for a fraction of sequences in
a group. Subsequently, a hypergeometric test was used to provide statistical significance
between the count of alignments in an entire group and only significant members of the group.

| | | | | | 15,587 Most Significant | | | | |
|---|---|---|---|---|---|---|---|---|---|
| | TOTAL | ALL PROBED | | | Probes | | | Improvement | |
| | GENOMIC | | promoter | | | promoter | | in count of | Hyper- |
| Family | N | N | alignments | % | N | alignments | % | alignments | geo pval |
| FLI-L1 | 145 | 87 | 86 | 98.85% | 9 | 9 | 100.00% | 1.15% | 0.00E+00 |
| L1HS | 1696 | 294 | 280 | 95.24% | 43 | 43 | 100.00% | 4.76% | 0.00E+00 |
| L1PA2 | 4666 | 727 | 672 | 92.43% | 115 | 111 | 96.52% | 4.09% | 1.53E−02 |
| L1PA3 | 10281 | 1070 | 972 | 90.84% | 226 | 218 | 96.46% | 5.62% | 8.39E−05 |
| L1PA4 | 11462 | 540 | 429 | 79.44% | 118 | 107 | 90.68% | 11.23% | 7.57E−05 |
| L1PA5 | 10904 | 328 | 178 | 54.27% | 46 | 37 | 80.43% | 26.17% | 1.62E−05 |
| L1PA6 | 5617 | 169 | 62 | 36.69% | 13 | 8 | 61.54% | 24.85% | 1.41E−02 |
| L1PA7 | 12334 | 245 | 32 | 13.06% | 23 | 3 | 13.04% | (0.02%) | 3.25E−01 |
| L1PA8 | 7715 | 159 | 8 | 5.03% | 20 | 0 | 0.00% | (5.03%) | 6.68E−01 |

An additional level of analysis, shown in the Table 11B involved measurement of the level of homology of sequences near the 5′ end of each L1 sequence with an exemplar sequence represented by the first 700 bases of an active LINE-1 element of the class FLI-L1, which contain an active promoter. Using BLAT (Kent, 2002), those 5′-end sequences of different subclasses of L1PA elements scoring with a homology of 80% or better were selected. The table shows that among the L1PA elements present in the subset of the 15,587 most significant probes, there is a much higher percentage of sequences with good homology matches to the active L1 exemplar. This indicates that possible selection in tumors for demethylated L1 elements could involve specific features of the sequence at the 5′-end of the element, which harbor potential forward promoter as well as antisense promoter activity (discussed in section 3.9, below). If, for any given class of elements (i.e. L1PA5), a potentially active promoter exists, it may be more likely to be associated with a full-length L1PA5 elements. Along this line of thought, the apparent length-selection could be a by-product of functional promoter selection.

ix. Functional Significance of the Enrichment of Demethylated Line-1 Elements in Tumors

The simplest interpretation of the age-stratified dysregulation DNA methylation of repetitive DNA observed among normal tissue, non-tumoral adjacent tissue and tumors is that the younger members of repetitive DNA families are the most likely to be transcribed, and that these RNA transcripts are best able in normal cells to trigger RNA-directed chromatin silencing. Silencing efficiency would be additionally enhanced in the younger elements due to reduced sequence divergence, as recently proposed by Reiss and Mager (2007). Paradoxically, for the very youngest members of retrotransposon families, exemplified in the data set by SVA-F and full-length, active LINE-1s, the emergence of optimal silencing may still remain incomplete, for lack of sufficient evolutionary time for RNA-mediated silencing traits to be selected and fixed. Such a hypothesis could explain why the very youngest, human specific retrotransposon families are relatively under-methylated in normal tissue, as compared to their relatively older and more “mature” primate siblings. It has been reported that heterochromatic piRNA loci interact with potentially active transposons in Drosophila resulting in transposon control (Brennecke et al., 2007). Normal transcriptional events involving retrotransposon sequences occur in human oocytes (Georgiu et al, 2009) and are well documented in the murine germ-line, where DNA is transiently demethylated, and where piRNAs have been implicated in reestablishing silencing (Aravin et al., 2007, 2008, Kuramochi-Miyagawa, 2008). Unfortunately, the general understanding of the evolutionary history of piRNAs remains extremely limited, particularly with regards to the mechanism responsible for generation of new functional piRNA sequences, as novel subclasses of retrotransposons enter the genome.

The most recently evolved repetitive elements can have accumulated fewer mutations or truncations deleterious to their function, and their selective loss of epigenetic silencing could be associated with functions that increase the fitness of tumors, therefore subjecting to positive selection. An example of such a function would be the transcriptional activation of genes with oncogenic potential as a result of loss of methylation of cryptic promoter or enhancer sequences within a full-length retrotransposon. For example, Roman-Gomez et al (2005) reported that L1 hypomethylation led to activation of c-MET gene transcription driven by an L1 antisense promoter (Speek, 2001, Nigumann et al, 2002) located within intron one of the c-MET gene in patients with blast crisis chronic myeloid leukemia (BC-CML), where these transcriptional events may contribute to disease progression. More recently, Lin et al. (2006) reported the induction of an abnormal chimeric transcript in esophageal adenocarcinomas, initiated from the antisense promoter located in the 5′-UTR of a full-length LINE-1 element. Another function that could be subject to positive selection in tumor cell lineages is the transcriptional activation of a retrotransposon ORF coding for a reverse transcriptase. It has been reported that the reverse transcriptase inhibitor efavirenz antagonizes the growth of H69 human small-cell lung carcinomas in nude mice (Sinibaldi-Vallebona et al., 2005). The same group has recently reported that inhibition of the reverse transcriptase messenger RNA of LINE-1 elements or HERV-K elements leads to loss of tumorigenic potential in cell lines (Oriccio et al., 2007). Of course, an important caveat is that the reported occurrence in cancer cells of transcripts or proteins derived from retrotransposons could be merely coincidental, not causal.

An interesting functional hypothesis regarding L1 retrotransposon sequences is the possible unselfish participation of expressed and reverse-transcribed LINE-1 elements in nonstandard DNA double strand break repair in the context of oncogenesis, where normal repair mechanisms are disrupted (Helleday et al., 2007). Repair of double-strand breaks by gene conversion involving different endogenous LINE-1 elements has been reported in the mouse (Tremblay et al., 2000). DNA repair by endonuclease-independent LINE-1 retrotransposition was first reported by Morrish et al. (2002, see commentary by Eickbush, 2002) using a model reporter vector transfected into CHO cells. This pathway was found to be dependent on reverse transcriptase activity, and resulted in integration of a truncated LINE-1 sequence lacking target site duplications. Recently Sen et al. (2007) characterized sites in the human genome where L1 elements have integrated without signs of endonuclease-related activity, and found that the structural features of these loci suggested that they arose by double-strand break repair, resulting in translocations or deletions. Also relevant are the findings of Srikanta et al (2009), who scanned the human, chimpanzee, and rhesus macaque genomes, and reported 23 instances of Alu integration events most likely mediated by endonuclease-independent DNA repair (EIDR). Observations of truncated LINE-1 insertions in the context of physiological stress have been reported in two mouse models, lambda-MYC lymphomas and endogenous oxidative stress caused by deficient G6PD expression. In these two models (Rockwood et al, 2004), the LINE-1 insertions, plausibly generated by the EIDR mechanism, have been captured within a chromosomally integrated lac-Z reporter vector. The observed insertions represent predominantly incomplete elements, and their frequency (25% of all events) is higher than the frequency of LINE-1 sequences in the mouse genome (10%).

EIDR involving LINE-1 and Alu elements could be ubiquitous in human cancer cells, and can have adaptive value, enhancing the viability of DNA repair-deficient tumor cells. The rapid rate of progress in high-throughput, low cost DNA sequencing will make it possible to sequence a large number of human tumor genomes to elucidate the sequences found at sites of genomic rearrangements, insertions, and deletions (CGP, 2009). Emerging genome analysis tools will also facilitate the design of experiments to assess the potential adaptive value of EIDR mediated by retroelements.

4. Conclusions

A novel microarray method for analysis of DNA methylation, based on the use of methylation sensitive as well as methylation dependent endonucleases, enables the interrogation of methylation levels in all compartments of the genome, including repetitive elements. Analysis of a substantial set of samples of squamous carcinomas of the head and neck, as well as non-tumor adjacent tissue and normal controls, reveals a complex framework of epigenetic dysregulation, where loss of methylation differentially affect distinct families of repetitive elements. Predominantly the younger, primate-specific members of retroelement families suffer the most dramatic loss of methylation, with the exception of some extremely young, human-specific retroelements. These complex patterns of differential susceptibility to disruption of silencing are probably a result of the natural history of evolutionary domestication of retroelements in genomes, in interplay with a minimal time requirement for strong silencing to be established. Primate-specific subfamilies of LINE-1 elements appear to suffer a particularly pronounced loss of methylation in tumors, with the most dramatic changes apparently observed for those primate retroelements with conserved promoter regions and longer sequences.

### C. Example 3

### Obtaining Status Biomarker Cancer Risk Score Metrics from a Human Buccal Sample Using a Microarray-Based Assay

A buccal sample can be obtained from the cheek of a subject using the “Buccal DNA Sample Collection Kit” (Bode Technologies). The DNA can be processed with two sets of different restriction endonucleases (methylation sensitive, or methylation dependent), and then amplified with phi29 DNA polymerase as described (Szpakowski et al, 2009).

The sample can be applied to a Nimblegen DNA microarray containing a set of DNA oligonucleotide probes, each 50 bases long, representing a genomic sampling for 25 different repetitive element families. Optionally, the probes can be 60, or 70, or 80, or 90 bases long. On the average each repetitive element family comprises from 30 to several thousand unique probe sequences, designed to be complementary to different specific loci in the genome. Each probe is replicated 4 times to allow for the calculation of the standard deviation of each probe measurement. Thus, the total number of probes in a microarray sector is 25×120×4=12,000. The microarray contains 24 sectors, permitting the analysis of 24 buccal samples at once. The total number of probes in the chip is 24×12,000=288,000.

Probe sets: The probe list can be specified by 25 families, chosen from a master set of 138 repetitive element families (Table 1), which are known to yield good classification results. The coordinates of all probes in all 138 families is listed in Table 15.

TABLE 1
List of Top 138 classifier categories in rank order.

| | |
|---|---|
| 1. | LTR54B |
| 2. | MER67D |
| 3. | MER11B |
| 4. | MER6 |
| 5. | ERVL |
| 6. | U1 |
| 7. | MER34B |
| 8. | MER66C |
| 9. | HUERS-P3 |
| 10. | LTR56 |
| 11. | MLT1G1 |
| 12. | THE1B-int |
| 13. | HERV9 |
| 14. | MER4D |
| 15. | LTR14C |
| 16. | MLT2D |
| 17. | HERVFH21 |
| 18. | THE1B |
| 19. | LTR6B |
| 20. | MLT1A1 |
| 21. | LTR46 |
| 22. | centr |
| 23. | Charlie5 |
| 24. | MLT1D-int |
| 25. | MLT2B3 |
| 26. | MER50B |
| 27. | HERVK11 |
| 28. | MER70A |
| 29. | Charlie3 |
| 30. | PABL_B |
| 31. | MER50 |
| 32. | MSR1 |
| 33. | AluYa5/8 |
| 34. | LTR2 |
| 35. | LTR10B |
| 36. | MLT1A |
| 37. | HERVK22 |
| 38. | HERVL |
| 39. | GSAT |
| 40. | LTR33A |
| 41. | LTR10B1 |
| 42. | MSTB-int |
| 43. | Cheshire |
| 44. | LTR17 |
| 45. | LTR51 |
| 46. | MSTA |
| 47. | MER11A |
| 48. | MER51B |
| 49. | MLT2B2 |
| 50. | SVA |
| 51. | SVA_A |
| 52. | SVA_B |
| 53. | SVA_C |
| 54. | SVA_D |
| 55. | SVA_E |
| 56. | SVA_F |
| 57. | L1PA12 |
| 58. | MSTC |
| 59. | ERVL-B4 |
| 60. | LTR9B |
| 61. | HERVK14 |
| 62. | LTR14B |
| 63. | HUERS-P2 |
| 64. | LTR29 |
| 65. | LTR6A |
| 66. | MSTB1 |
| 67. | ALR/Alpha |
| 68. | MSTD |
| 69. | LTR48B |
| 70. | LTR52 |
| 71. | LTR8 |
| 72. | MER105 |
| 73. | LTR8A |
| 74. | MER67A |
| 75. | HUERS-P1 |
| 76. | MER92B |
| 77. | LTR22 |
| 78. | LTR7B |
| 79. | L1PB1 |
| 80. | MER51A |
| 81. | L1PA15-16 |
| 82. | LTR36 |
| 83. | LTR28 |
| 84. | PABL_A |
| 85. | LTR45B |
| 86. | MER4D1 |
| 87. | AcHobo |
| 88. | LTR7Y |
| 89. | HERVL18 |
| 90. | LTR48 |
| 91. | LTR30 |
| 92. | MLT1A0 |
| 93. | HERVK9 |
| 94. | LTR1B |
| 95. | LTR45C |
| 96. | MSTB |
| 97. | LTR47A |
| 98. | MER11D |
| 99. | LTR19A |
| 100. | THE1C |
| 101. | LTR66 |
| 102. | MLT1E2 |
| 103. | MER115 |
| 104. | SST1 |
| 105. | MER34B-int |
| 106. | LTR65 |
| 107. | MER34C |
| 108. | MER44D |
| 109. | MER57A-int |
| 110. | MLT2B1 |
| 111. | L1PA10 |
| 112. | MER4A1 |
| 113. | MER6A |
| 114. | MLT1E |
| 115. | MER41E |
| 116. | MLT2B4 |
| 117. | 7SK |
| 118. | HERVP71A |
| 119. | L1MA7 |
| 120. | L1PBa1 |
| 121. | LTR5 |
| 122. | MER44C |
| 123. | GSATII |
| 124. | THE1D |
| 125. | L1MA1 |
| 126. | LTR7 |
| 127. | LTR9 |
| 128. | MER63A |
| 129. | MER91C |
| 130. | LTR5A |
| 131. | Harlequin |
| 132. | L1PB4 |
| 133. | MLT1F1 |
| 134. | L1M3f |
| 135. | MLT1F |
| 136. | MLT2A2 |
| 137. | LTR14 |
| 138. | MER11C |

The microarray can be subject to a hybridization protocol, and the microarray signals can be processed using bioinformatics protocols as described by Szpakowski et al., 2009.

A Random Forest binary tree classifier can be used to process the data (Strobl et al., 2009), yielding a classification result. The classifier assigns the sample to one of the three following categories: Normal, Tumor, Non-tumor tissue-at-risk.

The list of top 138 Classifier Categories (repetitive element families) in order of rank is as follows: LTR54B, MER67D, MER11B, MER6, ERVL, U1, MER34B, MER66C, HUERS-P3, LTR56, MLT1G1, THE1B-int, HERV9, MER4D, LTR14C, MLT2D, HERVFH21, THE1B, LTR6B, MLT1A1, LTR46, centr, Charlie5, MLT1D-int, MLT2B3, MER50B, HERVK11, MER70A, Charlie3, PABL_B, MER50, MSR1, AluYa5/8, LTR2, LTR10B, MLT1A, HERVK22, HERVL, GSAT, LTR33A, LTR10B1, MSTB-int, Cheshire, LTR17, LTR51, MSTA, MER11A, MER51B, MLT2B2, SVA, SVA_A, SVA_B, SVA_C, SVA_D, SVA_E, SVA_F, L1PA12, MSTC, ERVL-B4, LTR9B, HERVK14, LTR14B, HUERS-P2, LTR29, LTR6A, MSTB1, ALR/Alpha, MSTD, LTR48B, LTR52, LTR8, MER105, LTR8A, MER67A, HUERS-P1, MER92B, LTR22, LTR7B, L1PB1, MER51A, L1PA15-16, LTR36, LTR28, PABL_A, LTR45B, MER4D1, AcHobo, LTR7Y, HERVL18, LTR48, LTR30, MLT1A0, HERVK9, LTR1B, LTR45C, MSTB, LTR47A, MER11D, LTR19A, THE1C, LTR66, MLT1E2, MER115, SST1, MER34B-int, LTR65, MER34C, MER44D, MER57A-int, MLT2B1, L1PA10, MER4A1, MER6A, MLT1E, MER41E, MLT2B4, 7SK, HERVP71A, L1MA7, L1PBa1, LTR5, MER44C, GSATII, THE1D, L1MA1, LTR7, LTR9, MER63A, MER91C, LTR5A, Harlequin, L1PB4, MLT1F1, L1M3f, MLT1F, MLT2A2, LTR14, MER11C.

### D. Example 4

### Obtaining Status Biomarker Risk Score Metrics from a Human Buccal Sample Using a PCR-Based Assay

A buccal sample can be obtained from the cheek of a subject using the “Buccal DNA Sample Collection Kit” (Bode Technologies, Inc.). The DNA can be processed with sodium bisulfite using the Zymo EZ DNA Methylation-Gold kit (Zymo Research, Inc.).

The bisulfite-modified sample can be divided into 12 aliquots and each aliquot can be amplified by PCR using a specific pair of 12 sets of primers. For each primer pair, one primer can be anchored on a repeat family, chosen from among 138 informative families (see list in Example 3). The primer can be designed by obtaining the set of DNA sequences comprising the repeat family, and aligning the sequences with the program ClustalW (available at the website ch.embnet.org/software/ClustalW.html). The second primer can be anchored on an AluY repeat consensus sequence specific for AluY elements. The AluY consensus can be obtained by aligning a limited set of 150 randomly chosen AluY sequences with the program ClustalW.

The amplified DNA can be analyzed using a method capable of indirectly reporting the predicted level of methylated cytosines present of at CpG dinucleotide positions prior to bisulfite treatment, which converts cytosine to uridine, but does not convert methylcytosine. A preferred method, due to its low cost, is electrochemical detection (ECD, Nakahara et al., 1992) of cytosine and thymidine. The ratio of cytosine to thymidine can be converted to a relative DNA methylation level. An alternative method that can be used to obtain the ratio of cytosine to thymidine is Nanopore DNA sequencing (Clarke et al, 2009).

A Random Forest binary tree classifier can be used to process the data (Strobl et al., 2009), yielding a classification result. The classifier assigns the sample to one of the three following categories: Normal, Tumor, Non-tumor tissue-at-risk.

### E. Example 5

### Obtaining Status Biomarker Risk Score Metrics from a Human Buccal Sample Using 5-methyl-C-Binding Selection and Quantitative PCR

A buccal sample can be obtained from the cheek of a subject using the “Buccal DNA Sample Collection Kit” (Bode Technologies, Inc.). The DNA can be sheared by nebularization. It can then be immobilized using an antibody column, using an antibody capable of binding specifically 5 methyl cytosine. Alternatives to using a methyl-binding antibody are using either the MBD1 or the MECP2 methyl-binding proteins to immobilize the methylated DNA. This step (Sorensen & Collas, 2009) removes methylated DNA from solution, releasing an unmethylated DNA fraction. The immobilized, methylated DNA can then be recovered from the methyl-bindings column.

After 5-methyl-C-content separation, the methylated and the unmethylated DNA samples can be divided into 12 aliquots and each aliquot is amplified by quantitative PCR (as indicated in the next paragraph) using a specific pair of 12 sets of primers. For each primer pair, one primer can be anchored on a repeat family, chosen from among 138 informative families (Table 1). The primer can be designed by obtaining the set of DNA sequences comprising the repeat family, and aligning the sequences with the program ClustalW (available at the website ch.embnet.org/software/ClustalW.html). The second primer can be anchored on an AluY repeat consensus sequence specific for AluY elements. The AluY consensus can be obtained by aligning a limited set of 150 randomly chosen AluY sequences with the program ClustalW.

The amount of methylated and unmethylated DNA is determined using nanoliter-microarray quantitative PCR (Morrison et al., 2006; Dixon et al., 2009). This analytical format contains 3072 individual PCR reaction features, and enables the analysis of samples from 64 individuals, in quadruplicate, using specific primer pairs that measure the levels of 12 different repetitive element families.

A Random Forest binary tree classifier is used to process the data (Strobl et al., 2009), yielding a classification result. The classifier assigns the sample to one of the three following categories: Normal, Tumor, Non-tumor tissue-at-risk.

### F. Example 6

### Obtaining Status Biomarker Risk Score Metrics and Genetic State Data from a Human Buccal Sample Using Oligonucleotide-Mediated DNA Capture, Followed by DNA Sequencing Using a Pacific Biosciences SMRT System

A buccal sample can be obtained from the cheek of a subject using the “Buccal DNA Sample Collection Kit” (Bode Technologies, Inc.). The DNA from target repetitive element loci can be captured (Gnirke et al, 2009) using several long oligonucleotides (with a few degenerate base positions) specific for a consensus DNA sequence of each of 20 different repetitive element families. The degenerate positions enable binding of repetitive DNA at positions where the consensus sequence is imperfect. In this example, the 20 families are: LTR54B, MER11B, MER34B, LTR56, THE1B, HERV9, LTR14C, HERVFH21, LTR6B, LTR46, MLT1D, MER67D, HERVK11, LTR10B, HERVK22, MER6, MER66C, MLT1G1, MER4D, MLTD2. The repetitive element families used for sequence capture comprises 20 families, chosen from a master set of 138 repetitive element families (Table 1), which are known to yield good classification results. The coordinates of all probes in all 138 families is listed in Table 15.

The captured material can be released from the capture oligonucleotides, and the released DNA can be sequenced using the Pacific Biosciences SMRT system (Flusberg et al., 2010), which is capable of distinguishing cytosine from methylcytosine. The amount of DNA methylation can be calculated using the sequence data.

A Random Forest binary tree classifier can be used to process the data (Strobl et al., 2009), yielding a classification result. The classifier assigns the sample to one of the three following categories: Normal, Tumor, Non-tumor tissue-at-risk.

Due to the fact that the Pacific Biosciences single-molecule real-time SMRT system is capable of producing of long sequence reads, the data generated in this example will contain information about single-nucleotide polymorphisms (SNPs) present in the captured DNA loci. The base present at each SNP position in the sequenced locus will be different in different individuals being tested by this method. Thus, in this example data can be generated that specifies the Genetic State for some of the status biomarkers.

### G. Example 7

### Obtaining Status Biomarker Risk Score Metrics and Genetic State Data from a Human Buccal Sample Using Two Consecutive Steps of Oligonucleotide-Mediated DNA Capture, Followed by DNA Sequencing Using a Pacific Biosciences SMRT System

A buccal sample can be obtained from the cheek of a subject using the “Buccal DNA Sample Collection Kit” (Bode Technologies, Inc.). The DNA from target repetitive element loci can be captured (Gnirke et al, 2009) using several long oligonucleotides (with a few degenerate base positions) specific for a consensus DNA sequence of each of 20 different repetitive element families. The degenerate positions enable binding of repetitive DNA at positions where the consensus sequence is imperfect. In this example, the 20 families are: LTR54B, MER11B, MER34B, LTR56, THE1B, HERV9, LTR14C, HERVFH21, LTR6B, LTR46, MLT1D, MER67D, HERVK11, LTR10B, HERVK22, MER6, MER66C, MLT1G1, MER4D, and MLTD2. The repetitive element families used for sequence capture comprises 20 families, chosen from a master set of 138 repetitive element families (Table 1), which are known to yield good classification results. The coordinates of all probes in all 138 families is listed in Table 15.

The captured material can be released, and then re-captured (Gnirke et al, 2009), using a second set of several capture oligonucleotides specific for a consensus sequence for AluY and another set of consensus sequences for AluSx, AluSp, AluSg and AluSc repetitive elements. This can result in binding of DNA containing one repetitive element from the first set of 20, as well as a neighboring AluY or AluSx or AluSp or AluSg or AluSc elements.

The twice-captured material can be released from the capture oligonucleotides, and the released DNA can be sequenced using the Pacific Biosciences SMRT system (Flusberg et al., 2010), which is capable of distinguishing cytosine from methylcytosine. The amount of DNA methylation can be calculated using the sequence data.

A Random Forest binary tree classifier can be used to process the data (Strobl et al., 2009), yielding a classification result. The classifier assigns the sample to one of the three following categories: Normal, Tumor, Non-tumor tissue-at-risk.

Due to the fact that the Pacific Biosciences single-molecule real-time SMRT system is capable of producing of long sequence reads, the data generated in this example will contain information about single-nucleotide polymorphisms (SNPs) present in the captured DNA loci. The base present at each SNP position in the sequenced locus will be different in different individuals being tested by this method. Thus, in this example data can be generated that specifies the Genetic State for some of the status biomarkers.

### H. Example 8

A set of DNA methylation biomarkers that are informative regarding the stability of the genome and the epigenome in tissues are disclosed. The biomarkers were discovered through statistical analysis of a data set generated by microarrays that sampled the entire human genome, and included probes for gene promoters, non-gene-non-repetitive probes, and repetitive element probes.

The original set of microarray data comprised a list of 139,379 variables including gene probes, unique probes and repetitive element probes. In order to improve the robustness of the DNA methylation metrics, a strategy was developed whereby the probes belonging to the set of “repetitive elements” were subdivided in a total of 901 categories, based on their membership in specific sub-families of repetitive elements. For example, the 49 probes in the microarray mapping to a MER67D repetitive element (MER67D is a member of the LTR class of repetitive elements) were placed in one of the 901 categories, and the DNA methylation values of the 49 probes for that specific category were averaged. Repetitive element categories represented by less than 30 probes were not included in the set of 901 categories. The average methylation value of each of the 901 categories was used to perform a 3-way classification of normal tissue, vs. tumor tissue, vs. nontumor margin tissue.

Presented herein is a detailed report on a new set of classification experiments, performed with a subset of variables of higher quality. Using several sets of technical replicates of microarray experiments, a subset of the 901 category variables was selected based on a defined threshold value of the standard deviation the calculated values for each category, in 3 sets of 3 technical replicates. The subset of variable where the coefficient of variation was no larger than 15% was then selected. This variance quality filter reduced the number of variables to 569 categories. Table 18 is a list of these variables (repetitive DNA sequence families; status biomarkers).

A classification experiment using a Random Forest (RF) binary decision tree algorithm (Breiman, 2001) using the 569 repeat category variables was performed. The error rate in this analysis was 13% mis-classification. A list of the top 75 classifier variables was generated, which comprise categories of repeats according to the results of the RF classifier.

A classification experiment was then performed using a Support Vector Machine (SVM, Vapnik, 1998, Guyon et. al, 2002) classifier run using 569 variables. A list of the top 75 classifier variables was generated, which comprise categories of repeats according to the results of the SVM analysis. The performance of the SVM classifier was tested using top variables only, and found the best performance (100% accuracy) using either the top 18 or the top 19 variables.

Finally, the top 50 variables were ranked by the SVM classifier, and used in a Random Forest classification run. The error rate in this RF run was 8.1% mis-classification. This indicates that the SVM can be more effective than the RF algorithm in surveying all the available 569 variables to find the best classifiers among these variables.

1. Analysis of the Genomic Loci Comprising the Top Classifier Variables

The genomic organization of the repetitive elements that comprise the top variables in the classifiers was examined. It was observed that the genomic loci comprising the best classifiers have a structure characterized by the presence of two or three different repetitive elements, co-existing within a DNA window of approximately 500 to 1000 bases. A common organizational theme is a combination of an element belonging to the LTR family of retrotransposons, and an element belonging to the AluY (Young Alu) or AluSx family of retrotransposons. This information is presented in Table 14.

In the majority of cases, the LTR retrotransposon comprising a top classifier variable belongs to a primate-specific family, implying a relatively recent evolutionary origin. A small set of highly-performing variables consists of DNA transposons, such as Charlie3_MER1 and Charlie5_MER1, and Cheshire MER1 which have a different evolutionary origin. Yet another set of variables comprises repetitive sequences belonging to centromeric DNA, such as mini-satellite repeat 1 (MSR1), Gamma-satellite DNA, and Alpha-ALR-satellite DNA.

The presence of two or even three different repetitive element sequences within a window of 500 to 1000 bases may have biological consequences, for example a tendency of these loci to undergo loss of epigenetic silencing when cells are under stress, such as oxidative stress or cytokine-induce stress. Additionally, it is well known that centromeric sequences, which comprise closely spaced DNA repeats, are subject to loss of methylation under conditions of cellular stress.

TABLE 15
List of Coordinates of Probes for Top 138 Status Biomarkers
(Repetitive DNA Sequence Families) based on human genome sequence
HG_16 (NCBI, Build 34 (HG16), information available at website
ncbi.nlm.nih.gov/mapview/stats/
BuildStats.cgi?taxid=9606&build=34&ver=2; downloadable
from hgdownload.cse.ucsc.edu/goldenPath/hg16/bigZips/).

| | | |
|---|---|---|
| Chromosome | Start | End |

Table 15A. Repeat Category 7SK

| | | |
|---|---|---|
| chr17 | 71257544 | 71257594 |
| chr10 | 91588180 | 91588230 |
| chr19 | 38045695 | 38045745 |
| chr19 | 60940555 | 60940605 |
| chr1 | 20405463 | 20405513 |
| chr1 | 92963690 | 92963740 |
| chr1 | 160297183 | 160297233 |
| chr1 | 165128378 | 165128428 |
| chr20 | 38075670 | 38075720 |
| chr20 | 46173367 | 46173417 |
| chr22 | 35694072 | 35694122 |
| chr22 | 41192174 | 41192224 |
| chrX | 11993608 | 11993658 |
| chr2 | 27852249 | 27852299 |
| chr2 | 47561290 | 47561340 |
| chr2 | 61403203 | 61403253 |
| chr2 | 76646860 | 76646910 |
| chr2 | 112856736 | 112856786 |
| chr2 | 158357809 | 158357859 |
| chr2 | 161915873 | 161915923 |
| chr2 | 206968141 | 206968191 |
| chr3 | 73501367 | 73501417 |
| chr3 | 158056235 | 158056285 |
| chr4 | 47597771 | 47597821 |
| chr4 | 47597708 | 47597758 |
| chr4 | 87716288 | 87716338 |
| chr4 | 110995665 | 110995715 |
| chr4 | 122441323 | 122441373 |
| chr4 | 183452025 | 183452075 |
| chr5 | 6901137 | 6901187 |
| chr5 | 29994535 | 29994585 |
| chr5 | 124762901 | 124762951 |
| chr5 | 143943593 | 143943643 |
| chr6 | 13655995 | 13656045 |
| chr6 | 38161009 | 38161059 |
| chr6 | 39555238 | 39555288 |
| chr6 | 39555317 | 39555367 |
| chr6 | 106398279 | 106398329 |
| chr7 | 2782798 | 2782848 |
| chr7 | 69875282 | 69875332 |
| chr8 | 102106856 | 102106906 |
| chr8 | 127792715 | 127792765 |
| chr8 | 144729511 | 144729561 |
| chr9 | 128240427 | 128240477 |
| chr12 | 10128218 | 10128268 |
| chr12 | 42120864 | 42120914 |
| chr12 | 79239912 | 79239962 |
| chr12 | 111117041 | 111117091 |
| chr13 | 95299622 | 95299672 |
| chr13 | 97556227 | 97556277 |
| chr13 | 108265516 | 108265566 |
| chr14 | 88168864 | 88168914 |
| chr10 | 64643241 | 64643291 |
| chr15 | 94759559 | 94759609 |
| chr16 | 61552446 | 61552496 |
| chr16 | 67131812 | 67131862 |
| chr16 | 81775188 | 81775238 |
| chr17 | 34360776 | 34360826 |

Table 15B. Repeat Category AcHobo

| | | |
|---|---|---|
| chr17 | 38033648 | 38033698 |
| chr17 | 38344004 | 38344054 |
| chr17 | 39149510 | 39149560 |
| chr17 | 42615039 | 42615089 |
| chr17 | 46256291 | 46256341 |
| chr17 | 46294369 | 46294419 |
| chr17 | 46679812 | 46679862 |
| chr17 | 47504094 | 47504144 |
| chr17 | 48146243 | 48146293 |
| chr17 | 49075140 | 49075190 |
| chr17 | 49074938 | 49074988 |
| chr17 | 49206680 | 49206730 |
| chr17 | 54432218 | 54432268 |
| chr10 | 86898106 | 86898156 |
| chr17 | 66782871 | 66782921 |
| chr17 | 68048910 | 68048960 |
| chr17 | 70613320 | 70613370 |
| chr10 | 88647234 | 88647284 |
| chr17 | 75567525 | 75567575 |
| chr17 | 75711693 | 75711743 |
| chr17 | 76070843 | 76070893 |
| chr17 | 76385061 | 76385111 |
| chr17 | 76796859 | 76796909 |
| chr17 | 77036236 | 77036286 |
| chr17 | 77036010 | 77036060 |
| chr17 | 77982432 | 77982482 |
| chr17 | 78525870 | 78525920 |
| chr17 | 78527383 | 78527433 |
| chr17 | 78846446 | 78846496 |
| chr17 | 78912489 | 78912539 |
| chr17 | 78912378 | 78912428 |
| chr10 | 91717659 | 91717709 |
| chr10 | 91725026 | 91725076 |
| chr10 | 92494429 | 92494479 |
| chr17 | 79992627 | 79992677 |
| chr17 | 80671667 | 80671717 |
| chr18 | 3647883 | 3647933 |
| chr18 | 6293746 | 6293796 |
| chr18 | 10916207 | 10916257 |
| chr18 | 15113896 | 15113946 |
| chr18 | 20466119 | 20466169 |
| chr18 | 27660595 | 27660645 |
| chr18 | 27998944 | 27998994 |
| chr10 | 96960292 | 96960342 |
| chr18 | 43228144 | 43228194 |
| chr18 | 43612571 | 43612621 |
| chr18 | 44541327 | 44541377 |
| chr18 | 46566300 | 46566350 |
| chr18 | 53985474 | 53985524 |
| chr18 | 54338110 | 54338160 |
| chr18 | 54663374 | 54663424 |
| chr18 | 54819342 | 54819392 |
| chr18 | 58471019 | 58471069 |
| chr18 | 58938483 | 58938533 |
| chr18 | 59367070 | 59367120 |
| chr18 | 62321171 | 62321221 |
| chr18 | 67314825 | 67314875 |
| chr18 | 73759407 | 73759457 |
| chr18 | 74511608 | 74511658 |
| chr19 | 438082 | 438132 |
| chr10 | 99838254 | 99838304 |
| chr10 | 99838182 | 99838232 |
| chr19 | 1596543 | 1596593 |
| chr10 | 101189818 | 101189868 |
| chr19 | 4085549 | 4085599 |
| chr19 | 4595931 | 4595981 |
| chr19 | 4681103 | 4681153 |
| chr19 | 4738227 | 4738277 |
| chr19 | 5727319 | 5727369 |
| chr19 | 7079091 | 7079141 |
| chr19 | 7135546 | 7135596 |
| chr19 | 7606787 | 7606837 |
| chr19 | 7917845 | 7917895 |
| chr19 | 13470559 | 13470609 |
| chr19 | 14292747 | 14292797 |
| chr10 | 102974097 | 102974147 |
| chr19 | 16580710 | 16580760 |
| chr19 | 16581183 | 16581233 |
| chr19 | 16642234 | 16642284 |
| chr10 | 103595788 | 103595838 |
| chr19 | 22559892 | 22559942 |
| chr19 | 37379860 | 37379910 |
| chr19 | 37379641 | 37379691 |
| chr19 | 37744604 | 37744654 |
| chr19 | 40384870 | 40384920 |
| chr19 | 43909308 | 43909358 |
| chr19 | 50507087 | 50507137 |
| chr19 | 50507255 | 50507305 |
| chr19 | 55191494 | 55191544 |
| chr19 | 55477378 | 55477428 |
| chr10 | 112439269 | 112439319 |
| chr19 | 56503997 | 56504047 |
| chr19 | 59791392 | 59791442 |
| chr10 | 6059521 | 6059571 |
| chr19 | 60145473 | 60145523 |
| chr19 | 60512126 | 60512176 |
| chr19 | 61172002 | 61172052 |
| chr19 | 61369087 | 61369137 |
| chr19 | 61673912 | 61673962 |
| chr19 | 61673765 | 61673815 |
| chr19 | 62206915 | 62206965 |
| chr1 | 1219130 | 1219180 |
| chr1 | 2222080 | 2222130 |
| chr10 | 115289131 | 115289181 |
| chr1 | 6424683 | 6424733 |
| chr1 | 8853924 | 8853974 |
| chr1 | 8886972 | 8887022 |
| chr1 | 9179315 | 9179365 |
| chr1 | 10311115 | 10311165 |
| chr1 | 10397455 | 10397505 |
| chr1 | 10696981 | 10697031 |
| chr1 | 11042462 | 11042512 |
| chr1 | 13900746 | 13900796 |
| chr1 | 14530795 | 14530845 |
| chr1 | 17387367 | 17387417 |
| chr1 | 17395065 | 17395115 |
| chr1 | 17981043 | 17981093 |
| chr1 | 19584604 | 19584654 |
| chr1 | 21945039 | 21945089 |
| chr1 | 22727204 | 22727254 |
| chr1 | 24033094 | 24033144 |
| chr1 | 24064272 | 24064322 |
| chr1 | 26371003 | 26371053 |
| chr1 | 35664897 | 35664947 |
| chr1 | 36098518 | 36098568 |
| chr1 | 38810163 | 38810213 |
| chr1 | 39126439 | 39126489 |
| chr1 | 39601783 | 39601833 |
| chr1 | 39798956 | 39799006 |
| chr1 | 43212208 | 43212258 |
| chr1 | 43528788 | 43528838 |
| chr10 | 122329373 | 122329423 |
| chr1 | 46858225 | 46858275 |
| chr1 | 50188781 | 50188831 |
| chr1 | 53285438 | 53285488 |
| chr1 | 54846596 | 54846646 |
| chr1 | 62160833 | 62160883 |
| chr1 | 66577709 | 66577759 |
| chr10 | 124286273 | 124286323 |
| chr1 | 71292743 | 71292793 |
| chr1 | 81550129 | 81550179 |
| chr10 | 125085398 | 125085448 |
| chr1 | 86088966 | 86089016 |
| chr1 | 86089283 | 86089333 |
| chr1 | 90821623 | 90821673 |
| chr1 | 94055018 | 94055068 |
| chr1 | 109893113 | 109893163 |
| chr1 | 111070660 | 111070710 |
| chr1 | 111070711 | 111070761 |
| chr10 | 126161462 | 126161512 |
| chr1 | 113615663 | 113615713 |
| chr1 | 116751098 | 116751148 |
| chr10 | 126558072 | 126558122 |
| chr1 | 147926436 | 147926486 |
| chr1 | 147965385 | 147965435 |
| chr1 | 149875317 | 149875367 |
| chr1 | 149875082 | 149875132 |
| chr1 | 153293045 | 153293095 |
| chr1 | 153351320 | 153351370 |
| chr1 | 153416440 | 153416490 |
| chr1 | 154089424 | 154089474 |
| chr1 | 159571798 | 159571848 |
| chr1 | 172605308 | 172605358 |
| chr1 | 174157811 | 174157861 |
| chr1 | 174157721 | 174157771 |
| chr10 | 129340694 | 129340744 |
| chr1 | 180484076 | 180484126 |
| chr1 | 184302182 | 184302232 |
| chr1 | 198128206 | 198128256 |
| chr10 | 130052346 | 130052396 |
| chr1 | 201953707 | 201953757 |
| chr1 | 201953594 | 201953644 |
| chr1 | 202558286 | 202558336 |
| chr10 | 130466232 | 130466282 |
| chr1 | 208377402 | 208377452 |
| chr1 | 216809255 | 216809305 |
| chr1 | 217611296 | 217611346 |
| chr1 | 224597813 | 224597863 |
| chr1 | 225382647 | 225382697 |
| chr1 | 227014811 | 227014861 |
| chr1 | 228032360 | 228032410 |
| chr1 | 231333978 | 231334028 |
| chr1 | 242201154 | 242201204 |
| chr1 | 243774736 | 243774786 |
| chr1 | 243818801 | 243818851 |
| chr20 | 1639037 | 1639087 |
| chr20 | 2359815 | 2359865 |
| chr10 | 132655907 | 132655957 |
| chr20 | 4794544 | 4794594 |
| chr10 | 132875864 | 132875914 |
| chr20 | 8352707 | 8352757 |
| chr20 | 8785045 | 8785095 |
| chr20 | 12744038 | 12744088 |
| chr20 | 15219245 | 15219295 |
| chr20 | 16331983 | 16332033 |
| chr20 | 17421547 | 17421597 |
| chr20 | 18167373 | 18167423 |
| chr20 | 19256468 | 19256518 |
| chr20 | 25016578 | 25016628 |
| chr20 | 31517721 | 31517771 |
| chr20 | 33981949 | 33981999 |
| chr20 | 35663411 | 35663461 |
| chr20 | 44664779 | 44664829 |
| chr20 | 46788847 | 46788897 |
| chr20 | 47731514 | 47731564 |
| chr20 | 49171062 | 49171112 |
| chr20 | 49991611 | 49991661 |
| chr20 | 50477256 | 50477306 |
| chr20 | 52090981 | 52091031 |
| chr20 | 53032794 | 53032844 |
| chr20 | 56171631 | 56171681 |
| chr20 | 56315136 | 56315186 |
| chr20 | 58741009 | 58741059 |
| chr10 | 134482282 | 134482332 |
| chr21 | 18205559 | 18205609 |
| chr21 | 19124225 | 19124275 |
| chr21 | 22237492 | 22237542 |
| chr21 | 22274860 | 22274910 |
| chr11 | 296451 | 296501 |
| chr11 | 356079 | 356129 |
| chr21 | 31772150 | 31772200 |
| chr21 | 32549101 | 32549151 |
| chr21 | 33502480 | 33502530 |
| chr21 | 35314962 | 35315012 |
| chr21 | 35314819 | 35314869 |
| chr21 | 39201648 | 39201698 |
| chr21 | 40375173 | 40375223 |
| chr21 | 41654572 | 41654622 |
| chr21 | 41654818 | 41654868 |
| chr21 | 41951798 | 41951848 |
| chr21 | 42394531 | 42394581 |
| chr21 | 42499339 | 42499389 |
| chr21 | 43620913 | 43620963 |
| chr21 | 43639350 | 43639400 |
| chr21 | 45172222 | 45172272 |
| chr21 | 45172062 | 45172112 |
| chr22 | 16032014 | 16032064 |
| chr22 | 16838133 | 16838183 |
| chr22 | 16838034 | 16838084 |
| chr22 | 21248399 | 21248449 |
| chr22 | 23891448 | 23891498 |
| chr22 | 23950631 | 23950681 |
| chr22 | 25487588 | 25487638 |
| chr22 | 30548668 | 30548718 |
| chr11 | 2006284 | 2006334 |
| chr22 | 33347827 | 33347877 |
| chr22 | 34212852 | 34212902 |
| chr22 | 35961346 | 35961396 |
| chr22 | 36399697 | 36399747 |
| chr22 | 36715021 | 36715071 |
| chr22 | 37945312 | 37945362 |
| chr22 | 41814512 | 41814562 |
| chr22 | 41890927 | 41890977 |
| chr22 | 43686217 | 43686267 |
| chr22 | 45222376 | 45222426 |
| chr22 | 45222121 | 45222171 |
| chr22 | 46437500 | 46437550 |
| chr22 | 47274931 | 47274981 |
| chr22 | 48728252 | 48728302 |
| chr22 | 48865760 | 48865810 |
| chr22 | 48958556 | 48958606 |
| chrX | 3103984 | 3104034 |
| chrX | 5405075 | 5405125 |
| chrX | 5734438 | 5734488 |
| chrX | 11555492 | 11555542 |
| chrX | 16080155 | 16080205 |
| chrX | 19972661 | 19972711 |
| chrX | 23329497 | 23329547 |
| chrX | 29927182 | 29927232 |
| chrX | 38684803 | 38684853 |
| chrX | 38704294 | 38704344 |
| chrX | 38704229 | 38704279 |
| chrX | 44239240 | 44239290 |
| chrX | 50271543 | 50271593 |
| chr11 | 8044274 | 8044324 |
| chr11 | 8044169 | 8044219 |
| chrX | 53298965 | 53299015 |
| chrX | 61925053 | 61925103 |
| chrX | 69321553 | 69321603 |
| chrX | 70352118 | 70352168 |
| chrX | 76112402 | 76112452 |
| chrX | 94539704 | 94539754 |
| chrX | 95347195 | 95347245 |
| chrX | 98993968 | 98994018 |
| chrX | 101735540 | 101735590 |
| chrX | 105786269 | 105786319 |
| chrX | 107833581 | 107833631 |
| chrX | 108601103 | 108601153 |
| chrX | 117392119 | 117392169 |
| chrX | 117393347 | 117393397 |
| chr11 | 10587163 | 10587213 |
| chrX | 121777001 | 121777051 |
| chrX | 128957508 | 128957558 |
| chrX | 132294706 | 132294756 |
| chrX | 143188096 | 143188146 |
| chrX | 147321813 | 147321863 |
| chrX | 151545850 | 151545900 |
| chrX | 151545792 | 151545842 |
| chrX | 152678366 | 152678416 |
| chrX | 152900238 | 152900288 |
| chr2 | 2083196 | 2083246 |
| chr2 | 2083087 | 2083137 |
| chr2 | 3145169 | 3145219 |
| chr2 | 8708283 | 8708333 |
| chr2 | 9363992 | 9364042 |
| chr2 | 11844620 | 11844670 |
| chr2 | 12877875 | 12877925 |
| chr2 | 16118983 | 16119033 |
| chr2 | 16393967 | 16394017 |
| chr2 | 20014907 | 20014957 |
| chr2 | 20719209 | 20719259 |
| chr2 | 20719060 | 20719110 |
| chr2 | 20769963 | 20770013 |
| chr2 | 28539646 | 28539696 |
| chr2 | 31340723 | 31340773 |
| chr2 | 32967345 | 32967395 |
| chr2 | 33072704 | 33072754 |
| chr2 | 36725407 | 36725457 |
| chr2 | 39407514 | 39407564 |
| chr2 | 39593484 | 39593534 |
| chr2 | 40768414 | 40768464 |
| chr2 | 41198646 | 41198696 |
| chr2 | 43007362 | 43007412 |
| chr2 | 46853446 | 46853496 |
| chr11 | 21730809 | 21730859 |
| chr2 | 53966566 | 53966616 |
| chr2 | 53966387 | 53966437 |
| chr2 | 61059735 | 61059785 |
| chr2 | 61902549 | 61902599 |
| chr2 | 64108613 | 64108663 |
| chr2 | 65942101 | 65942151 |
| chr2 | 66836054 | 66836104 |
| chr2 | 71782940 | 71782990 |
| chr2 | 71912756 | 71912806 |
| chr2 | 75913248 | 75913298 |
| chr2 | 75914672 | 75914722 |
| chr2 | 77900691 | 77900741 |
| chr2 | 80220066 | 80220116 |
| chr2 | 85451994 | 85452044 |
| chr2 | 88328065 | 88328115 |
| chr11 | 28567205 | 28567255 |
| chr2 | 98943490 | 98943540 |
| chr2 | 100982445 | 100982495 |
| chr2 | 100982243 | 100982293 |
| chr2 | 102305463 | 102305513 |
| chr2 | 102405188 | 102405238 |
| chr2 | 105762941 | 105762991 |
| chr2 | 105764544 | 105764594 |
| chr2 | 106830104 | 106830154 |
| chr2 | 109263678 | 109263728 |
| chr11 | 32523448 | 32523498 |
| chr10 | 11930284 | 11930334 |
| chr10 | 11931290 | 11931340 |
| chr2 | 122681427 | 122681477 |
| chr2 | 132067385 | 132067435 |
| chr2 | 132938842 | 132938892 |
| chr2 | 151600316 | 151600366 |
| chr2 | 158255464 | 158255514 |
| chr11 | 34936132 | 34936182 |
| chr2 | 166687797 | 166687847 |
| chr2 | 167546064 | 167546114 |
| chr2 | 167545960 | 167546010 |
| chr2 | 169344452 | 169344502 |
| chr2 | 177783711 | 177783761 |
| chr2 | 180737244 | 180737294 |
| chr2 | 181333785 | 181333835 |
| chr2 | 188943352 | 188943402 |
| chr2 | 197051413 | 197051463 |
| chr2 | 201364123 | 201364173 |
| chr2 | 201956362 | 201956412 |
| chr2 | 202193112 | 202193162 |
| chr2 | 203041019 | 203041069 |
| chr2 | 203095986 | 203096036 |
| chr11 | 40409480 | 40409530 |
| chr2 | 206690687 | 206690737 |
| chr2 | 207618146 | 207618196 |
| chr2 | 208866270 | 208866320 |
| chr2 | 209227646 | 209227696 |
| chr2 | 212561548 | 212561598 |
| chr2 | 220465513 | 220465563 |
| chr2 | 230717019 | 230717069 |
| chr2 | 232444962 | 232445012 |
| chr2 | 234359000 | 234359050 |
| chr2 | 234359472 | 234359522 |
| chr2 | 235635960 | 235636010 |
| chr2 | 240417573 | 240417623 |
| chr2 | 240638557 | 240638607 |
| chr2 | 242278366 | 242278416 |
| chr3 | 3802307 | 3802357 |
| chr3 | 5032774 | 5032824 |
| chr3 | 11992030 | 11992080 |
| chr3 | 16195042 | 16195092 |
| chr3 | 23890216 | 23890266 |
| chr3 | 24005272 | 24005322 |
| chr3 | 27086328 | 27086378 |
| chr3 | 29437246 | 29437296 |
| chr3 | 32129335 | 32129385 |
| chr3 | 33972706 | 33972756 |
| chr3 | 35786197 | 35786247 |
| chr3 | 35786085 | 35786135 |
| chr3 | 37712576 | 37712626 |
| chr3 | 39197339 | 39197389 |
| chr3 | 39528768 | 39528818 |
| chr3 | 39528612 | 39528662 |
| chr3 | 43073401 | 43073451 |
| chr3 | 43072935 | 43072985 |
| chr3 | 48714884 | 48714934 |
| chr3 | 53172979 | 53173029 |
| chr3 | 55338444 | 55338494 |
| chr3 | 62349113 | 62349163 |
| chr3 | 65768491 | 65768541 |
| chr3 | 65793710 | 65793760 |
| chr3 | 67581763 | 67581813 |
| chr3 | 72639734 | 72639784 |
| chr11 | 58108017 | 58108067 |
| chr3 | 101235957 | 101236007 |
| chr3 | 101244428 | 101244478 |
| chr3 | 101374368 | 101374418 |
| chr11 | 59255927 | 59255977 |
| chr3 | 107914675 | 107914725 |
| chr3 | 109297348 | 109297398 |
| chr3 | 110201641 | 110201691 |
| chr3 | 113397077 | 113397127 |
| chr3 | 114617897 | 114617947 |
| chr3 | 115113418 | 115113468 |
| chr10 | 12728262 | 12728312 |
| chr11 | 60413422 | 60413472 |
| chr11 | 60413164 | 60413214 |
| chr11 | 60600501 | 60600551 |
| chr3 | 128126644 | 128126694 |
| chr3 | 133026334 | 133026384 |
| chr3 | 133399293 | 133399343 |
| chr3 | 141768396 | 141768446 |
| chr3 | 142496080 | 142496130 |
| chr3 | 143896380 | 143896430 |
| chr3 | 155378298 | 155378348 |
| chr3 | 160793497 | 160793547 |
| chr3 | 161214134 | 161214184 |
| chr3 | 172832364 | 172832414 |
| chr3 | 173686615 | 173686665 |
| chr3 | 186759221 | 186759271 |
| chr3 | 190263915 | 190263965 |
| chr3 | 191615997 | 191616047 |
| chr3 | 195391268 | 195391318 |
| chr3 | 195391474 | 195391524 |
| chr3 | 198433839 | 198433889 |
| chr4 | 632936 | 632986 |
| chr4 | 1512252 | 1512302 |
| chr4 | 5629895 | 5629945 |
| chr4 | 5977763 | 5977813 |
| chr4 | 6429501 | 6429551 |
| chr4 | 6621795 | 6621845 |
| chr4 | 6743719 | 6743769 |
| chr4 | 8743364 | 8743414 |
| chr4 | 10302606 | 10302656 |
| chr4 | 11498615 | 11498665 |
| chr4 | 11631193 | 11631243 |
| chr4 | 17182682 | 17182732 |
| chr4 | 20885144 | 20885194 |
| chr4 | 25284538 | 25284588 |
| chr4 | 25867838 | 25867888 |
| chr4 | 33743107 | 33743157 |
| chr4 | 35750293 | 35750343 |
| chr4 | 37430378 | 37430428 |
| chr4 | 37518264 | 37518314 |
| chr4 | 38687965 | 38688015 |
| chr4 | 40585741 | 40585791 |
| chr4 | 46426251 | 46426301 |
| chr4 | 46827049 | 46827099 |
| chr4 | 46828432 | 46828482 |
| chr4 | 75497606 | 75497656 |
| chr4 | 77296608 | 77296658 |
| chr4 | 79926832 | 79926882 |
| chr11 | 65577159 | 65577209 |
| chr4 | 86218993 | 86219043 |
| chr4 | 98289148 | 98289198 |
| chr4 | 104217720 | 104217770 |
| chr10 | 13514337 | 13514387 |
| chr4 | 110762298 | 110762348 |
| chr4 | 124731975 | 124732025 |
| chr11 | 66435761 | 66435811 |
| chr4 | 130080378 | 130080428 |
| chr4 | 141349395 | 141349445 |
| chr4 | 141775631 | 141775681 |
| chr4 | 142857644 | 142857694 |
| chr4 | 147748273 | 147748323 |
| chr4 | 154619764 | 154619814 |
| chr4 | 158890212 | 158890262 |
| chr4 | 167213535 | 167213585 |
| chr4 | 172984608 | 172984658 |
| chr4 | 179124771 | 179124821 |
| chr4 | 179979455 | 179979505 |
| chr4 | 185364069 | 185364119 |
| chr4 | 185364243 | 185364293 |
| chr4 | 185364500 | 185364550 |
| chr4 | 185718954 | 185719004 |
| chr4 | 185989038 | 185989088 |
| chr4 | 185989243 | 185989293 |
| chr4 | 186894580 | 186894630 |
| chr11 | 68303328 | 68303378 |
| chr4 | 188362110 | 188362160 |
| chr4 | 188362485 | 188362535 |
| chr4 | 188362567 | 188362617 |
| chr11 | 68335353 | 68335403 |
| chr4 | 190491093 | 190491143 |
| chr4 | 191221781 | 191221831 |
| chr5 | 2320972 | 2321022 |
| chr11 | 68915383 | 68915433 |
| chr11 | 69591881 | 69591931 |
| chr11 | 69591678 | 69591728 |
| chr11 | 69667991 | 69668041 |
| chr5 | 26019454 | 26019504 |
| chr5 | 31792469 | 31792519 |
| chr5 | 50234969 | 50235019 |
| chr5 | 53204858 | 53204908 |
| chr5 | 54209024 | 54209074 |
| chr5 | 55329670 | 55329720 |
| chr5 | 58207802 | 58207852 |
| chr5 | 59093596 | 59093646 |
| chr5 | 60237819 | 60237869 |
| chr11 | 70341686 | 70341736 |
| chr5 | 64757319 | 64757369 |
| chr5 | 66182604 | 66182654 |
| chr5 | 66182420 | 66182470 |
| chr5 | 72248492 | 72248542 |
| chr5 | 72248935 | 72248985 |
| chr5 | 76741345 | 76741395 |
| chr5 | 77678295 | 77678345 |
| chr5 | 80159780 | 80159830 |
| chr5 | 85612020 | 85612070 |
| chr5 | 96028341 | 96028391 |
| chr5 | 96029344 | 96029394 |
| chr5 | 108014312 | 108014362 |
| chr5 | 129714940 | 129714990 |
| chr5 | 130712724 | 130712774 |
| chr5 | 137757510 | 137757560 |
| chr5 | 139259670 | 139259720 |
| chr5 | 139652667 | 139652717 |
| chr5 | 145245185 | 145245235 |
| chr5 | 150355183 | 150355233 |
| chr5 | 150355252 | 150355302 |
| chr11 | 73971842 | 73971892 |
| chr5 | 157027162 | 157027212 |
| chr5 | 157928299 | 157928349 |
| chr5 | 157978767 | 157978817 |
| chr5 | 158248853 | 158248903 |
| chr10 | 14723787 | 14723837 |
| chr5 | 168927991 | 168928041 |
| chr5 | 172017491 | 172017541 |
| chr5 | 173538983 | 173539033 |
| chr5 | 173538914 | 173538964 |
| chr5 | 173799261 | 173799311 |
| chr5 | 174009991 | 174010041 |
| chr5 | 174318368 | 174318418 |
| chr5 | 177860099 | 177860149 |
| chr5 | 178204069 | 178204119 |
| chr5 | 179910114 | 179910164 |
| chr5 | 180600261 | 180600311 |
| chr6 | 641769 | 641819 |
| chr6 | 1015494 | 1015544 |
| chr6 | 2878482 | 2878532 |
| chr6 | 10680631 | 10680681 |
| chr6 | 12115748 | 12115798 |
| chr6 | 14163046 | 14163096 |
| chr6 | 14787740 | 14787790 |
| chr6 | 14789978 | 14790028 |
| chr6 | 14998714 | 14998764 |
| chr6 | 15320745 | 15320795 |
| chr6 | 15336890 | 15336940 |
| chr6 | 16503239 | 16503289 |
| chr6 | 18428310 | 18428360 |
| chr6 | 18430008 | 18430058 |
| chr6 | 18481550 | 18481600 |
| chr6 | 20238376 | 20238426 |
| chr6 | 29030132 | 29030182 |
| chr6 | 37008654 | 37008704 |
| chr6 | 37548665 | 37548715 |
| chr6 | 37548554 | 37548604 |
| chr6 | 42116756 | 42116806 |
| chr6 | 42178912 | 42178962 |
| chr6 | 42407656 | 42407706 |
| chr6 | 42544666 | 42544716 |
| chr6 | 48347126 | 48347176 |
| chr6 | 48827503 | 48827553 |
| chr6 | 49447343 | 49447393 |
| chr6 | 51786246 | 51786296 |
| chr11 | 87838099 | 87838149 |
| chr6 | 56963629 | 56963679 |
| chr6 | 63822591 | 63822641 |
| chr6 | 64171815 | 64171865 |
| chr6 | 64707417 | 64707467 |
| chr6 | 68186050 | 68186100 |
| chr6 | 69749351 | 69749401 |
| chr6 | 71138656 | 71138706 |
| chr11 | 91615420 | 91615470 |
| chr6 | 83153884 | 83153934 |
| chr6 | 107636826 | 107636876 |
| chr6 | 107637213 | 107637263 |
| chr6 | 107755005 | 107755055 |
| chr6 | 109045928 | 109045978 |
| chr11 | 94557072 | 94557122 |
| chr6 | 112729491 | 112729541 |
| chr6 | 114788654 | 114788704 |
| chr11 | 94857374 | 94857424 |
| chr6 | 122487214 | 122487264 |
| chr6 | 123110279 | 123110329 |
| chr6 | 127186452 | 127186502 |
| chr6 | 128522920 | 128522970 |
| chr6 | 128804568 | 128804618 |
| chr11 | 96788761 | 96788811 |
| chr6 | 138310191 | 138310241 |
| chr6 | 139796646 | 139796696 |
| chr6 | 147951537 | 147951587 |
| chr6 | 150520153 | 150520203 |
| chr6 | 152116579 | 152116629 |
| chr6 | 152295686 | 152295736 |
| chr6 | 158051939 | 158051989 |
| chr6 | 158848861 | 158848911 |
| chr6 | 162180352 | 162180402 |
| chr6 | 163530858 | 163530908 |
| chr6 | 163530781 | 163530831 |
| chr6 | 166119506 | 166119556 |
| chr6 | 167303587 | 167303637 |
| chr11 | 104824327 | 104824377 |
| chr6 | 168314321 | 168314371 |
| chr6 | 168860792 | 168860842 |
| chr7 | 1515542 | 1515592 |
| chr7 | 4279874 | 4279924 |
| chr7 | 5049792 | 5049842 |
| chr7 | 5056113 | 5056163 |
| chr10 | 16671458 | 16671508 |
| chr10 | 16671317 | 16671367 |
| chr7 | 10376052 | 10376102 |
| chr7 | 11776211 | 11776261 |
| chr7 | 12687068 | 12687118 |
| chr7 | 18752497 | 18752547 |
| chr11 | 111790318 | 111790368 |
| chr7 | 35932365 | 35932415 |
| chr7 | 38890776 | 38890826 |
| chr7 | 39524770 | 39524820 |
| chr7 | 42964743 | 42964793 |
| chr7 | 47277063 | 47277113 |
| chr7 | 47276843 | 47276893 |
| chr7 | 57546257 | 57546307 |
| chr7 | 67220930 | 67220980 |
| chr7 | 69388316 | 69388366 |
| chr11 | 116141133 | 116141183 |
| chr7 | 79354441 | 79354491 |
| chr7 | 87469330 | 87469380 |
| chr7 | 96278058 | 96278108 |
| chr7 | 96388066 | 96388116 |
| chr7 | 102124333 | 102124383 |
| chr7 | 104032274 | 104032324 |
| chr7 | 104032077 | 104032127 |
| chr7 | 105745327 | 105745377 |
| chr7 | 115683646 | 115683696 |
| chr7 | 120032332 | 120032382 |
| chr7 | 124669591 | 124669641 |
| chr7 | 128698268 | 128698318 |
| chr7 | 132326854 | 132326904 |
| chr7 | 146649075 | 146649125 |
| chr7 | 147746302 | 147746352 |
| chr7 | 148913230 | 148913280 |
| chr7 | 150355749 | 150355799 |
| chr7 | 150561164 | 150561214 |
| chr11 | 119503110 | 119503160 |
| chr7 | 154088238 | 154088288 |
| chr7 | 156739834 | 156739884 |
| chr7 | 157682380 | 157682430 |
| chr8 | 395160 | 395210 |
| chr8 | 852451 | 852501 |
| chr8 | 1842158 | 1842208 |
| chr8 | 1968966 | 1969016 |
| chr8 | 2728861 | 2728911 |
| chr8 | 2873020 | 2873070 |
| chr8 | 3858119 | 3858169 |
| chr8 | 4436771 | 4436821 |
| chr8 | 8489268 | 8489318 |
| chr8 | 9369462 | 9369512 |
| chr8 | 9779830 | 9779880 |
| chr8 | 11150840 | 11150890 |
| chr8 | 11397772 | 11397822 |
| chr8 | 14354201 | 14354251 |
| chr8 | 15955420 | 15955470 |
| chr8 | 16732596 | 16732646 |
| chr8 | 19853889 | 19853939 |
| chr8 | 21771256 | 21771306 |
| chr8 | 22697120 | 22697170 |
| chr8 | 24606508 | 24606558 |
| chr8 | 26083838 | 26083888 |
| chr8 | 26083748 | 26083798 |
| chr11 | 123405083 | 123405133 |
| chr8 | 28498904 | 28498954 |
| chr8 | 32218451 | 32218501 |
| chr8 | 33275743 | 33275793 |
| chr8 | 40157204 | 40157254 |
| chr11 | 124779706 | 124779756 |
| chr8 | 54283716 | 54283766 |
| chr8 | 67714386 | 67714436 |
| chr8 | 69892379 | 69892429 |
| chr8 | 72728256 | 72728306 |
| chr8 | 75309197 | 75309247 |
| chr8 | 78103048 | 78103098 |
| chr8 | 79921073 | 79921123 |
| chr8 | 83011030 | 83011080 |
| chr11 | 126454221 | 126454271 |
| chr8 | 89786569 | 89786619 |
| chr11 | 127775200 | 127775250 |
| chr8 | 98581455 | 98581505 |
| chr8 | 101184624 | 101184674 |
| chr8 | 101566448 | 101566498 |
| chr8 | 103507373 | 103507423 |
| chr8 | 104191762 | 104191812 |
| chr8 | 105356661 | 105356711 |
| chr8 | 105356415 | 105356465 |
| chr8 | 105964811 | 105964861 |
| chr8 | 113101231 | 113101281 |
| chr8 | 113494216 | 113494266 |
| chr8 | 113513869 | 113513919 |
| chr8 | 113820262 | 113820312 |
| chr8 | 122902045 | 122902095 |
| chr8 | 123409029 | 123409079 |
| chr8 | 127158839 | 127158889 |
| chr8 | 132193387 | 132193437 |
| chr8 | 133186232 | 133186282 |
| chr8 | 139246315 | 139246365 |
| chr8 | 140909259 | 140909309 |
| chr11 | 129855764 | 129855814 |
| chr8 | 142214170 | 142214220 |
| chr11 | 130453419 | 130453469 |
| chr9 | 2181442 | 2181492 |
| chr9 | 2181391 | 2181441 |
| chr9 | 3891195 | 3891245 |
| chr9 | 3891098 | 3891148 |
| chr9 | 4169590 | 4169640 |
| chr9 | 13954069 | 13954119 |
| chr9 | 29341931 | 29341981 |
| chr9 | 30629819 | 30629869 |
| chr9 | 37050530 | 37050580 |
| chr9 | 68289256 | 68289306 |
| chr9 | 74090361 | 74090411 |
| chr9 | 74440279 | 74440329 |
| chr9 | 77539477 | 77539527 |
| chr11 | 133642690 | 133642740 |
| chr9 | 84246172 | 84246222 |
| chr9 | 86380195 | 86380245 |
| chr9 | 86644643 | 86644693 |
| chr9 | 86644557 | 86644607 |
| chr11 | 133858765 | 133858815 |
| chr9 | 88567294 | 88567344 |
| chr9 | 89665152 | 89665202 |
| chr11 | 133943898 | 133943948 |
| chr9 | 92026656 | 92026706 |
| chr9 | 93525460 | 93525510 |
| chr9 | 93796554 | 93796604 |
| chr9 | 93796809 | 93796859 |
| chr9 | 93797357 | 93797407 |
| chr9 | 93823868 | 93823918 |
| chr9 | 93847190 | 93847240 |
| chr9 | 95871806 | 95871856 |
| chr9 | 96708262 | 96708312 |
| chr9 | 96896361 | 96896411 |
| chr9 | 119401902 | 119401952 |
| chr9 | 120932061 | 120932111 |
| chr9 | 122190184 | 122190234 |
| chr9 | 122380434 | 122380484 |
| chr9 | 125990956 | 125991006 |
| chr9 | 125992658 | 125992708 |
| chr12 | 1914064 | 1914114 |
| chr9 | 129190269 | 129190319 |
| chr9 | 129247319 | 129247369 |
| chr12 | 2265067 | 2265117 |
| chr9 | 129872736 | 129872786 |
| chr9 | 130013737 | 130013787 |
| chr9 | 131871970 | 131872020 |
| chr9 | 131908284 | 131908334 |
| chr9 | 132049608 | 132049658 |
| chr9 | 132828175 | 132828225 |
| chr9 | 132829494 | 132829544 |
| chr9 | 135389171 | 135389221 |
| chr10 | 21769230 | 21769280 |
| chr12 | 6080881 | 6080931 |
| chr12 | 6822395 | 6822445 |
| chr12 | 8018497 | 8018547 |
| chr12 | 12055922 | 12055972 |
| chr12 | 12332434 | 12332484 |
| chr12 | 15984830 | 15984880 |
| chr12 | 21202094 | 21202144 |
| chr10 | 24117679 | 24117729 |
| chr12 | 25861147 | 25861197 |
| chr12 | 29833202 | 29833252 |
| chr12 | 31495808 | 31495858 |
| chr12 | 32077403 | 32077453 |
| chr12 | 42181489 | 42181539 |
| chr10 | 26068046 | 26068096 |
| chr12 | 48771175 | 48771225 |
| chr12 | 48965188 | 48965238 |
| chr12 | 49631606 | 49631656 |
| chr12 | 49831262 | 49831312 |
| chr12 | 51217048 | 51217098 |
| chr12 | 51517349 | 51517399 |
| chr12 | 53161356 | 53161406 |
| chr12 | 56376938 | 56376988 |
| chr10 | 27865964 | 27866014 |
| chr12 | 60730709 | 60730759 |
| chr12 | 81126768 | 81126818 |
| chr10 | 29300672 | 29300722 |
| chr12 | 86107433 | 86107483 |
| chr12 | 86226095 | 86226145 |
| chr10 | 1211921 | 1211971 |
| chr12 | 93628898 | 93628948 |
| chr12 | 102015189 | 102015239 |
| chr12 | 103917345 | 103917395 |
| chr12 | 106105920 | 106105970 |
| chr12 | 108018559 | 108018609 |
| chr12 | 108591345 | 108591395 |
| chr10 | 30685839 | 30685889 |
| chr12 | 111560024 | 111560074 |
| chr12 | 112462044 | 112462094 |
| chr12 | 112498927 | 112498977 |
| chr12 | 114760939 | 114760989 |
| chr12 | 115227864 | 115227914 |
| chr12 | 118282932 | 118282982 |
| chr12 | 118583979 | 118584029 |
| chr12 | 119820254 | 119820304 |
| chr12 | 120049084 | 120049134 |
| chr12 | 123246905 | 123246955 |
| chr12 | 123559989 | 123560039 |
| chr12 | 126600641 | 126600691 |
| chr12 | 127285303 | 127285353 |
| chr12 | 127367738 | 127367788 |
| chr12 | 127770661 | 127770711 |
| chr12 | 128390825 | 128390875 |
| chr12 | 130671637 | 130671687 |
| chr12 | 131338987 | 131339037 |
| chr12 | 131653164 | 131653214 |
| chr12 | 131653341 | 131653391 |
| chr10 | 35498550 | 35498600 |
| chr13 | 20110580 | 20110630 |
| chr13 | 20110509 | 20110559 |
| chr13 | 20758061 | 20758111 |
| chr13 | 20870666 | 20870716 |
| chr13 | 22078920 | 22078970 |
| chr13 | 22861829 | 22861879 |
| chr13 | 23055379 | 23055429 |
| chr10 | 36624838 | 36624888 |
| chr13 | 25204692 | 25204742 |
| chr13 | 28523014 | 28523064 |
| chr13 | 29949747 | 29949797 |
| chr10 | 37156906 | 37156956 |
| chr13 | 32008367 | 32008417 |
| chr13 | 34830722 | 34830772 |
| chr13 | 35188419 | 35188469 |
| chr13 | 36508404 | 36508454 |
| chr13 | 39447396 | 39447446 |
| chr13 | 41132885 | 41132935 |
| chr13 | 41832624 | 41832674 |
| chr13 | 43487308 | 43487358 |
| chr13 | 48994804 | 48994854 |
| chr13 | 49686413 | 49686463 |
| chr13 | 57145435 | 57145485 |
| chr13 | 57146553 | 57146603 |
| chr13 | 67144550 | 67144600 |
| chr13 | 71221036 | 71221086 |
| chr13 | 101043270 | 101043320 |
| chr13 | 102036854 | 102036904 |
| chr13 | 109889142 | 109889192 |
| chr13 | 110464198 | 110464248 |
| chr13 | 111260615 | 111260665 |
| chr13 | 111260408 | 111260458 |
| chr13 | 112766290 | 112766340 |
| chr14 | 22748871 | 22748921 |
| chr14 | 23061541 | 23061591 |
| chr10 | 47797856 | 47797906 |
| chr14 | 33166741 | 33166791 |
| chr14 | 43114922 | 43114972 |
| chr14 | 48780466 | 48780516 |
| chr14 | 51129286 | 51129336 |
| chr10 | 50093323 | 50093373 |
| chr14 | 61435048 | 61435098 |
| chr14 | 61570986 | 61571036 |
| chr14 | 63653198 | 63653248 |
| chr14 | 64305800 | 64305850 |
| chr14 | 65920083 | 65920133 |
| chr14 | 68909480 | 68909530 |
| chr10 | 51779035 | 51779085 |
| chr14 | 71689608 | 71689658 |
| chr14 | 72667282 | 72667332 |
| chr14 | 79420434 | 79420484 |
| chr14 | 83710581 | 83710631 |
| chr14 | 83710423 | 83710473 |
| chr14 | 86702400 | 86702450 |
| chr14 | 89969026 | 89969076 |
| chr14 | 90677947 | 90677997 |
| chr14 | 90677853 | 90677903 |
| chr14 | 91758822 | 91758872 |
| chr14 | 93824686 | 93824736 |
| chr10 | 56326610 | 56326660 |
| chr14 | 94561222 | 94561272 |
| chr14 | 99644028 | 99644078 |
| chr14 | 102378023 | 102378073 |
| chr14 | 102652806 | 102652856 |
| chr14 | 102734285 | 102734335 |
| chr14 | 103096635 | 103096685 |
| chr15 | 20488360 | 20488410 |
| chr15 | 22718655 | 22718705 |
| chr10 | 61977266 | 61977316 |
| chr15 | 28911720 | 28911770 |
| chr15 | 32273533 | 32273583 |
| chr15 | 35167266 | 35167316 |
| chr15 | 39804196 | 39804246 |
| chr15 | 39832933 | 39832983 |
| chr15 | 41227887 | 41227937 |
| chr15 | 41462147 | 41462197 |
| chr15 | 47168802 | 47168852 |
| chr15 | 48791176 | 48791226 |
| chr10 | 64661455 | 64661505 |
| chr15 | 49992843 | 49992893 |
| chr15 | 50183004 | 50183054 |
| chr15 | 56196985 | 56197035 |
| chr15 | 59823806 | 59823856 |
| chr15 | 59835984 | 59836034 |
| chr15 | 59869013 | 59869063 |
| chr10 | 67240546 | 67240596 |
| chr15 | 64037577 | 64037627 |
| chr15 | 64037391 | 64037441 |
| chr15 | 71393499 | 71393549 |
| chr15 | 71620950 | 71621000 |
| chr15 | 72062248 | 72062298 |
| chr15 | 72992071 | 72992121 |
| chr15 | 73183032 | 73183082 |
| chr15 | 73810447 | 73810497 |
| chr15 | 75967763 | 75967813 |
| chr15 | 76922818 | 76922868 |
| chr15 | 78041941 | 78041991 |
| chr15 | 78047755 | 78047805 |
| chr15 | 78650328 | 78650378 |
| chr15 | 79007469 | 79007519 |
| chr15 | 82861186 | 82861236 |
| chr15 | 86897975 | 86898025 |
| chr15 | 87122532 | 87122582 |
| chr15 | 87806034 | 87806084 |
| chr15 | 90641648 | 90641698 |
| chr15 | 90937393 | 90937443 |
| chr15 | 96849263 | 96849313 |
| chr15 | 97888048 | 97888098 |
| chr15 | 99627216 | 99627266 |
| chr15 | 99626942 | 99626992 |
| chr16 | 3310854 | 3310904 |
| chr16 | 4408762 | 4408812 |
| chr16 | 4408662 | 4408712 |
| chr16 | 5794972 | 5795022 |
| chr16 | 12008746 | 12008796 |
| chr16 | 21094347 | 21094397 |
| chr16 | 21527933 | 21527983 |
| chr16 | 21553583 | 21553633 |
| chr16 | 25275696 | 25275746 |
| chr16 | 27355940 | 27355990 |
| chr16 | 30153978 | 30154028 |
| chr16 | 47955794 | 47955844 |
| chr16 | 47955886 | 47955936 |
| chr16 | 53331985 | 53332035 |
| chr16 | 57063801 | 57063851 |
| chr16 | 57422028 | 57422078 |
| chr16 | 59203191 | 59203241 |
| chr16 | 60719023 | 60719073 |
| chr16 | 68864711 | 68864761 |
| chr16 | 69335937 | 69335987 |
| chr10 | 74054424 | 74054474 |
| chr16 | 72434844 | 72434894 |
| chr16 | 73291357 | 73291407 |
| chr16 | 73460518 | 73460568 |
| chr16 | 74125051 | 74125101 |
| chr16 | 77728239 | 77728289 |
| chr16 | 78599619 | 78599669 |
| chr16 | 81315727 | 81315777 |
| chr16 | 81315556 | 81315606 |
| chr16 | 84058635 | 84058685 |
| chr10 | 3319173 | 3319223 |
| chr16 | 86205946 | 86205996 |
| chr16 | 87719830 | 87719880 |
| chr16 | 87976536 | 87976586 |
| chr16 | 88216477 | 88216527 |
| chr17 | 89168 | 89218 |
| chr17 | 1690348 | 1690398 |
| chr17 | 2824825 | 2824875 |
| chr17 | 3139463 | 3139513 |
| chr17 | 3634038 | 3634088 |
| chr17 | 3974531 | 3974581 |
| chr17 | 4632158 | 4632208 |
| chr17 | 5557942 | 5557992 |
| chr17 | 9842486 | 9842536 |
| chr17 | 9950959 | 9951009 |
| chr17 | 10074695 | 10074745 |
| chr17 | 28278019 | 28278069 |
| chr17 | 30393270 | 30393320 |
| chr10 | 81563691 | 81563741 |

Table 15C. Repeat Category ALR/Alpha

| | | |
|---|---|---|
| chr19 | 24288185 | 24288235 |
| chr20 | 26253566 | 26253616 |
| chrX | 57387835 | 57387885 |
| chrX | 60603335 | 60603385 |
| chrX | 60681504 | 60681554 |
| chrY | 9747477 | 9747527 |
| chr2 | 133188017 | 133188067 |
| chr2 | 133191550 | 133191600 |
| chr2 | 133199583 | 133199633 |
| chr11 | 48736956 | 48737006 |
| chr11 | 48841206 | 48841256 |
| chr11 | 51276257 | 51276307 |
| chr11 | 54695291 | 54695341 |
| chr3 | 90273606 | 90273656 |
| chr3 | 90304025 | 90304075 |
| chr3 | 90358338 | 90358388 |
| chr3 | 126704530 | 126704580 |
| chr4 | 8113471 | 8113521 |
| chr4 | 8537174 | 8537224 |
| chr5 | 46209983 | 46210033 |
| chr5 | 46251800 | 46251850 |
| chr5 | 46427072 | 46427122 |
| chr5 | 49502167 | 49502217 |
| chr7 | 61730603 | 61730653 |
| chr8 | 43561357 | 43561407 |
| chr8 | 46987008 | 46987058 |
| chr8 | 47399004 | 47399054 |
| chr9 | 62804300 | 62804350 |
| chr9 | 62814866 | 62814916 |
| chr9 | 63061394 | 63061444 |
| chr12 | 34717568 | 34717618 |
| chr12 | 36346642 | 36346692 |
| chr12 | 36464697 | 36464747 |
| chr12 | 36496322 | 36496372 |
| chr12 | 36563428 | 36563478 |
| chr12 | 36570252 | 36570302 |
| chr13 | 21796347 | 21796397 |
| chr16 | 35128111 | 35128161 |
| chr16 | 35133001 | 35133051 |
| chr17 | 25661821 | 25661871 |

Table 15D. Repeat Category AluYa5/8

| | | |
|---|---|---|
| chr19 | 46698982 | 46699032 |
| chr19 | 55111494 | 55111544 |
| chr1 | 170807816 | 170807866 |
| chr1 | 229139468 | 229139518 |
| chrX | 150808162 | 150808212 |
| chrY | 8369826 | 8369876 |
| chr2 | 15663196 | 15663246 |
| chr2 | 19652981 | 19653031 |
| chr2 | 20137243 | 20137293 |
| chr2 | 40308786 | 40308836 |
| chr2 | 119001256 | 119001306 |
| chr2 | 138549806 | 138549856 |
| chr2 | 192761922 | 192761972 |
| chr3 | 12544180 | 12544230 |
| chr3 | 41109763 | 41109813 |
| chr3 | 85146169 | 85146219 |
| chr11 | 60702157 | 60702207 |
| chr11 | 60702243 | 60702293 |
| chr3 | 160240573 | 160240623 |
| chr4 | 33841855 | 33841905 |
| chr5 | 148430151 | 148430201 |
| chr6 | 29440752 | 29440802 |
| chr6 | 100063559 | 100063609 |
| chr11 | 93925178 | 93925228 |
| chr6 | 110791169 | 110791219 |
| chr6 | 152132203 | 152132253 |
| chr11 | 112648006 | 112648056 |
| chr8 | 12779638 | 12779688 |
| chr8 | 92357226 | 92357276 |
| chr9 | 14215515 | 14215565 |
| chr16 | 59082167 | 59082217 |
| chr16 | 84662577 | 84662627 |
| chr16 | 89060522 | 89060572 |

Table 15E. Repeat Category centr

| | | |
|---|---|---|
| chr18 | 11179136 | 11179186 |
| chr19 | 24288185 | 24288235 |
| chr19 | 32817810 | 32817860 |
| chr19 | 32829565 | 32829615 |
| chr19 | 32833169 | 32833219 |
| chr19 | 32857592 | 32857642 |
| chr19 | 41439574 | 41439624 |
| chr19 | 41442157 | 41442207 |
| chr19 | 41450941 | 41450991 |
| chr19 | 41454843 | 41454893 |
| chr19 | 42190779 | 42190829 |
| chr19 | 42192956 | 42193006 |
| chr19 | 42195250 | 42195300 |
| chr19 | 42197755 | 42197805 |
| chr19 | 42238521 | 42238571 |
| chr19 | 42240257 | 42240307 |
| chr19 | 42241761 | 42241811 |
| chr19 | 42243830 | 42243880 |
| chr19 | 42452388 | 42452438 |
| chr19 | 42464712 | 42464762 |
| chr19 | 42467493 | 42467543 |
| chr19 | 42487646 | 42487696 |
| chr19 | 42732769 | 42732819 |
| chr19 | 42734420 | 42734470 |
| chr19 | 42734152 | 42734202 |
| chr1 | 140839821 | 140839871 |
| chr1 | 140841172 | 140841222 |
| chr1 | 140842296 | 140842346 |
| chr1 | 225694342 | 225694392 |
| chr20 | 25829405 | 25829455 |
| chr20 | 25832323 | 25832373 |
| chr20 | 25834137 | 25834187 |
| chr20 | 25834860 | 25834910 |
| chr20 | 25836178 | 25836228 |
| chr20 | 25837963 | 25838013 |
| chr20 | 25839822 | 25839872 |
| chr20 | 25841510 | 25841560 |
| chr20 | 25843969 | 25844019 |
| chr20 | 25960261 | 25960311 |
| chr20 | 25961445 | 25961495 |
| chr20 | 25962189 | 25962239 |
| chr20 | 25962783 | 25962833 |
| chr20 | 25963950 | 25964000 |
| chr20 | 25965913 | 25965963 |
| chr20 | 25967555 | 25967605 |
| chr20 | 26227263 | 26227313 |
| chr20 | 26253566 | 26253616 |
| chr20 | 29411493 | 29411543 |
| chr20 | 29413930 | 29413980 |
| chr20 | 29414479 | 29414529 |
| chr20 | 29416037 | 29416087 |
| chr20 | 29465353 | 29465403 |
| chr21 | 14367127 | 14367177 |
| chr22 | 14476328 | 14476378 |
| chr22 | 14476980 | 14477030 |
| chr22 | 14477878 | 14477928 |
| chr22 | 14478232 | 14478282 |
| chr22 | 15603677 | 15603727 |
| chrX | 57091205 | 57091255 |
| chrX | 57249652 | 57249702 |
| chrX | 57252055 | 57252105 |
| chrX | 57253098 | 57253148 |
| chrX | 57255768 | 57255818 |
| chrX | 57258572 | 57258622 |
| chrX | 57387835 | 57387885 |
| chrX | 60603335 | 60603385 |
| chrX | 60681504 | 60681554 |
| chrX | 139840254 | 139840304 |
| chrY | 8244873 | 8244923 |
| chrY | 8586928 | 8586978 |
| chrY | 8594455 | 8594505 |
| chrY | 8599812 | 8599862 |
| chrY | 8802608 | 8802658 |
| chrY | 8832966 | 8833016 |
| chrY | 9747477 | 9747527 |
| chrY | 22603191 | 22603241 |
| chrY | 22604888 | 22604938 |
| chrY | 22612413 | 22612463 |
| chrY | 22811477 | 22811527 |
| chrY | 22816605 | 22816655 |
| chrY | 22816405 | 22816455 |
| chr2 | 31403726 | 31403776 |
| chr2 | 132723394 | 132723444 |
| chr2 | 133188017 | 133188067 |
| chr2 | 133191550 | 133191600 |
| chr2 | 133199583 | 133199633 |
| chr11 | 48736956 | 48737006 |
| chr11 | 48841206 | 48841256 |
| chr11 | 51276257 | 51276307 |
| chr11 | 54695291 | 54695341 |
| chr3 | 90273606 | 90273656 |
| chr3 | 90304025 | 90304075 |
| chr3 | 90358338 | 90358388 |
| chr3 | 126704530 | 126704580 |
| chr3 | 174852694 | 174852744 |
| chr4 | 7062858 | 7062908 |
| chr4 | 8113471 | 8113521 |
| chr4 | 8537174 | 8537224 |
| chr4 | 49434782 | 49434832 |
| chr4 | 49435470 | 49435520 |
| chr4 | 133105673 | 133105723 |
| chr4 | 133108807 | 133108857 |
| chr4 | 133114432 | 133114482 |
| chr4 | 133119447 | 133119497 |
| chr4 | 133121931 | 133121981 |
| chr4 | 133128547 | 133128597 |
| chr4 | 133144611 | 133144661 |
| chr4 | 133356425 | 133356475 |
| chr5 | 46209983 | 46210033 |
| chr5 | 46251800 | 46251850 |
| chr5 | 46427072 | 46427122 |
| chr5 | 49502167 | 49502217 |
| chr5 | 177837223 | 177837273 |
| chr6 | 2582761 | 2582811 |
| chr6 | 2584919 | 2584969 |
| chr6 | 2584618 | 2584668 |
| chr7 | 57323292 | 57323342 |
| chr7 | 57484576 | 57484626 |
| chr7 | 57489252 | 57489302 |
| chr7 | 57491668 | 57491718 |
| chr7 | 57493544 | 57493594 |
| chr7 | 57608476 | 57608526 |
| chr7 | 57611298 | 57611348 |
| chr7 | 57612957 | 57613007 |
| chr7 | 57614872 | 57614922 |
| chr7 | 57657592 | 57657642 |
| chr7 | 57658603 | 57658653 |
| chr7 | 57660994 | 57661044 |
| chr7 | 61730603 | 61730653 |
| chr7 | 63740873 | 63740923 |
| chr7 | 63742146 | 63742196 |
| chr7 | 63742406 | 63742456 |
| chr7 | 63743064 | 63743114 |
| chr7 | 63745004 | 63745054 |
| chr7 | 63747477 | 63747527 |
| chr7 | 63750110 | 63750160 |
| chr7 | 63752506 | 63752556 |
| chr7 | 63755151 | 63755201 |
| chr7 | 63754991 | 63755041 |
| chr7 | 63756844 | 63756894 |
| chr8 | 43417880 | 43417930 |
| chr8 | 43418739 | 43418789 |
| chr8 | 43420187 | 43420237 |
| chr8 | 43421368 | 43421418 |
| chr8 | 43423341 | 43423391 |
| chr8 | 43436146 | 43436196 |
| chr8 | 43561357 | 43561407 |
| chr8 | 46947006 | 46947056 |
| chr8 | 46987008 | 46987058 |
| chr8 | 47032918 | 47032968 |
| chr8 | 47039361 | 47039411 |
| chr8 | 47042168 | 47042218 |
| chr8 | 47057029 | 47057079 |
| chr8 | 47063194 | 47063244 |
| chr8 | 47065152 | 47065202 |
| chr8 | 47069102 | 47069152 |
| chr8 | 47070617 | 47070667 |
| chr8 | 47074813 | 47074863 |
| chr8 | 47105806 | 47105856 |
| chr8 | 47110603 | 47110653 |
| chr8 | 47121169 | 47121219 |
| chr8 | 47122261 | 47122311 |
| chr8 | 47126219 | 47126269 |
| chr8 | 47129687 | 47129737 |
| chr8 | 47131011 | 47131061 |
| chr8 | 47136244 | 47136294 |
| chr8 | 47142204 | 47142254 |
| chr8 | 47144188 | 47144238 |
| chr8 | 47145885 | 47145935 |
| chr8 | 47147133 | 47147183 |
| chr8 | 47148295 | 47148345 |
| chr8 | 47157918 | 47157968 |
| chr8 | 47159194 | 47159244 |
| chr8 | 47162521 | 47162571 |
| chr8 | 47163587 | 47163637 |
| chr8 | 47164854 | 47164904 |
| chr8 | 47168722 | 47168772 |
| chr8 | 47171751 | 47171801 |
| chr8 | 47172827 | 47172877 |
| chr8 | 47177504 | 47177554 |
| chr8 | 47178335 | 47178385 |
| chr8 | 47183041 | 47183091 |
| chr8 | 47189273 | 47189323 |
| chr8 | 47189098 | 47189148 |
| chr8 | 47191211 | 47191261 |
| chr8 | 47204227 | 47204277 |
| chr8 | 47307555 | 47307605 |
| chr8 | 47335857 | 47335907 |
| chr8 | 47340466 | 47340516 |
| chr8 | 47344331 | 47344381 |
| chr8 | 47352798 | 47352848 |
| chr8 | 47359859 | 47359909 |
| chr8 | 47361658 | 47361708 |
| chr8 | 47365194 | 47365244 |
| chr8 | 47366498 | 47366548 |
| chr8 | 47375191 | 47375241 |
| chr8 | 47399004 | 47399054 |
| chr9 | 42227834 | 42227884 |
| chr9 | 42278036 | 42278086 |
| chr9 | 42279994 | 42280044 |
| chr9 | 42281227 | 42281277 |
| chr9 | 42282424 | 42282474 |
| chr9 | 42283864 | 42283914 |
| chr9 | 42285059 | 42285109 |
| chr9 | 42286324 | 42286374 |
| chr9 | 43172902 | 43172952 |
| chr9 | 43174537 | 43174587 |
| chr9 | 43176252 | 43176302 |
| chr9 | 43656896 | 43656946 |
| chr9 | 62804300 | 62804350 |
| chr9 | 62814866 | 62814916 |
| chr9 | 63061394 | 63061444 |
| chr9 | 63427466 | 63427516 |
| chr9 | 63959998 | 63960048 |
| chr9 | 64968296 | 64968346 |
| chr9 | 64971276 | 64971326 |
| chr12 | 34255417 | 34255467 |
| chr12 | 34333749 | 34333799 |
| chr12 | 34335653 | 34335703 |
| chr12 | 34337071 | 34337121 |
| chr12 | 34340101 | 34340151 |
| chr12 | 34344148 | 34344198 |
| chr12 | 34346821 | 34346871 |
| chr12 | 34348414 | 34348464 |
| chr12 | 34349452 | 34349502 |
| chr12 | 34351406 | 34351456 |
| chr12 | 34355131 | 34355181 |
| chr12 | 34356000 | 34356050 |
| chr12 | 34357433 | 34357483 |
| chr12 | 34361478 | 34361528 |
| chr12 | 34365891 | 34365941 |
| chr12 | 34368023 | 34368073 |
| chr12 | 34370145 | 34370195 |
| chr12 | 34371879 | 34371929 |
| chr12 | 34373142 | 34373192 |
| chr12 | 34375854 | 34375904 |
| chr12 | 34376598 | 34376648 |
| chr12 | 34381192 | 34381242 |
| chr12 | 34384507 | 34384557 |
| chr12 | 34386405 | 34386455 |
| chr12 | 34388750 | 34388800 |
| chr12 | 34391121 | 34391171 |
| chr12 | 34397175 | 34397225 |
| chr12 | 34398620 | 34398670 |
| chr12 | 34400674 | 34400724 |
| chr12 | 34402377 | 34402427 |
| chr12 | 34403978 | 34404028 |
| chr12 | 34407067 | 34407117 |
| chr12 | 34410197 | 34410247 |
| chr12 | 34411525 | 34411575 |
| chr12 | 34413190 | 34413240 |
| chr12 | 34415055 | 34415105 |
| chr12 | 34415954 | 34416004 |
| chr12 | 34418034 | 34418084 |
| chr12 | 34420630 | 34420680 |
| chr12 | 34421456 | 34421506 |
| chr12 | 34422708 | 34422758 |
| chr12 | 34423824 | 34423874 |
| chr12 | 34425483 | 34425533 |
| chr12 | 34427806 | 34427856 |
| chr12 | 34429072 | 34429122 |
| chr12 | 34436147 | 34436197 |
| chr12 | 34437394 | 34437444 |
| chr12 | 34442947 | 34442997 |
| chr12 | 34446019 | 34446069 |
| chr12 | 34447767 | 34447817 |
| chr12 | 34642395 | 34642445 |
| chr12 | 34644020 | 34644070 |
| chr12 | 34645336 | 34645386 |
| chr12 | 34646060 | 34646110 |
| chr12 | 34648711 | 34648761 |
| chr12 | 34649045 | 34649095 |
| chr12 | 34650600 | 34650650 |
| chr12 | 34717568 | 34717618 |
| chr12 | 36221876 | 36221926 |
| chr12 | 36223975 | 36224025 |
| chr12 | 36226413 | 36226463 |
| chr12 | 36227743 | 36227793 |
| chr12 | 36346642 | 36346692 |
| chr12 | 36464697 | 36464747 |
| chr12 | 36496322 | 36496372 |
| chr12 | 36563428 | 36563478 |
| chr12 | 36570252 | 36570302 |
| chr12 | 36832980 | 36833030 |
| chr12 | 36835270 | 36835320 |
| chr12 | 36837781 | 36837831 |
| chr12 | 36838599 | 36838649 |
| chr12 | 36840393 | 36840443 |
| chr13 | 17317122 | 17317172 |
| chr13 | 21796347 | 21796397 |
| chr16 | 35128111 | 35128161 |
| chr16 | 35133001 | 35133051 |
| chr16 | 35295200 | 35295250 |
| chr16 | 35456280 | 35456330 |
| chr16 | 35458498 | 35458548 |
| chr16 | 36108845 | 36108895 |
| chr16 | 36111212 | 36111262 |
| chr17 | 3875990 | 3876040 |
| chr17 | 21639309 | 21639359 |
| chr17 | 21639450 | 21639500 |
| chr17 | 21971245 | 21971295 |
| chr17 | 21971093 | 21971143 |
| chr17 | 21973380 | 21973430 |
| chr17 | 21975111 | 21975161 |
| chr17 | 21976921 | 21976971 |
| chr17 | 22047600 | 22047650 |
| chr17 | 22049889 | 22049939 |
| chr17 | 22051925 | 22051975 |
| chr17 | 22053384 | 22053434 |
| chr17 | 22054809 | 22054859 |
| chr17 | 22056570 | 22056620 |
| chr17 | 22337670 | 22337720 |
| chr17 | 25628588 | 25628638 |
| chr17 | 25631116 | 25631166 |
| chr17 | 25661821 | 25661871 |

Table 15F. Repeat Category Charlie3

| | | |
|---|---|---|
| chr18 | 58908877 | 58908927 |
| chr19 | 7920262 | 7920312 |
| chr19 | 13997832 | 13997882 |
| chr1 | 207583145 | 207583195 |
| chr1 | 226034555 | 226034605 |
| chr21 | 37679077 | 37679127 |
| chr22 | 43620446 | 43620496 |
| chr22 | 43623651 | 43623701 |
| chrX | 152657818 | 152657868 |
| chr11 | 17873993 | 17874043 |
| chr2 | 41959018 | 41959068 |
| chr2 | 95512906 | 95512956 |
| chr2 | 97646535 | 97646585 |
| chr2 | 135510711 | 135510761 |
| chr2 | 166219397 | 166219447 |
| chr2 | 239580686 | 239580736 |
| chr11 | 46193204 | 46193254 |
| chr3 | 126562363 | 126562413 |
| chr11 | 61176671 | 61176721 |
| chr3 | 159673119 | 159673169 |
| chr4 | 75451244 | 75451294 |
| chr4 | 83770076 | 83770126 |
| chr4 | 124198946 | 124198996 |
| chr5 | 480729 | 480779 |
| chr5 | 4784010 | 4784060 |
| chr5 | 6925473 | 6925523 |
| chr5 | 25338550 | 25338600 |
| chr5 | 27217373 | 27217423 |
| chr5 | 87979860 | 87979910 |
| chr10 | 14252993 | 14253043 |
| chr5 | 149177590 | 149177640 |
| chr6 | 13198886 | 13198936 |
| chr6 | 65420348 | 65420398 |
| chr6 | 151169241 | 151169291 |
| chr6 | 165807070 | 165807120 |
| chr6 | 165807440 | 165807490 |
| chr6 | 165807729 | 165807779 |
| chr6 | 165808686 | 165808736 |
| chr6 | 165809204 | 165809254 |
| chr6 | 166444880 | 166444930 |
| chr6 | 166448032 | 166448082 |
| chr11 | 108417617 | 108417667 |
| chr7 | 4736450 | 4736500 |
| chr7 | 15918159 | 15918209 |
| chr7 | 83415113 | 83415163 |
| chr8 | 8142313 | 8142363 |
| chr8 | 143922985 | 143923035 |
| chr9 | 1657487 | 1657537 |
| chr9 | 7852861 | 7852911 |
| chr12 | 88429056 | 88429106 |
| chr12 | 103268256 | 103268306 |
| chr14 | 65106140 | 65106190 |
| chr15 | 58372278 | 58372328 |
| chr16 | 55484499 | 55484549 |
| chr16 | 56739477 | 56739527 |
| chr16 | 57946441 | 57946491 |
| chr16 | 87357200 | 87357250 |

Table 15G. Repeat Category Charlie5

| | | |
|---|---|---|
| chr17 | 40876783 | 40876833 |
| chr17 | 58177286 | 58177336 |
| chr17 | 63015535 | 63015585 |
| chr17 | 66071272 | 66071322 |
| chr17 | 75033585 | 75033635 |
| chr17 | 75595937 | 75595987 |
| chr17 | 77023398 | 77023448 |
| chr17 | 77023196 | 77023246 |
| chr18 | 7150535 | 7150585 |
| chr18 | 7454774 | 7454824 |
| chr18 | 42954636 | 42954686 |
| chr10 | 98823723 | 98823773 |
| chr18 | 75438140 | 75438190 |
| chr19 | 974755 | 974805 |
| chr19 | 1341419 | 1341469 |
| chr19 | 4070800 | 4070850 |
| chr19 | 5696174 | 5696224 |
| chr19 | 9005140 | 9005190 |
| chr19 | 9298032 | 9298082 |
| chr19 | 9933526 | 9933576 |
| chr19 | 13161646 | 13161696 |
| chr19 | 14076728 | 14076778 |
| chr19 | 14500440 | 14500490 |
| chr10 | 103332745 | 103332795 |
| chr19 | 35269831 | 35269881 |
| chr19 | 41985506 | 41985556 |
| chr10 | 104831543 | 104831593 |
| chr19 | 45404999 | 45405049 |
| chr19 | 48894926 | 48894976 |
| chr19 | 52274575 | 52274625 |
| chr19 | 52274818 | 52274868 |
| chr19 | 53296313 | 53296363 |
| chr19 | 53351208 | 53351258 |
| chr19 | 54678826 | 54678876 |
| chr19 | 56543008 | 56543058 |
| chr19 | 56918269 | 56918319 |
| chr19 | 56918103 | 56918153 |
| chr19 | 56990359 | 56990409 |
| chr19 | 60471294 | 60471344 |
| chr1 | 1701418 | 1701468 |
| chr10 | 116022227 | 116022277 |
| chr1 | 11877501 | 11877551 |
| chr1 | 16091477 | 16091527 |
| chr1 | 32659795 | 32659845 |
| chr1 | 37504564 | 37504614 |
| chr10 | 123357697 | 123357747 |
| chr1 | 63901750 | 63901800 |
| chr1 | 63903549 | 63903599 |
| chr1 | 75971738 | 75971788 |
| chr1 | 75971582 | 75971632 |
| chr1 | 106030164 | 106030214 |
| chr1 | 113694570 | 113694620 |
| chr1 | 157371606 | 157371656 |
| chr1 | 158769859 | 158769909 |
| chr10 | 128663547 | 128663597 |
| chr1 | 168473090 | 168473140 |
| chr1 | 178228364 | 178228414 |
| chr1 | 179229739 | 179229789 |
| chr1 | 181150320 | 181150370 |
| chr1 | 195011566 | 195011616 |
| chr1 | 205138390 | 205138440 |
| chr1 | 210529927 | 210529977 |
| chr1 | 229920796 | 229920846 |
| chr1 | 232035771 | 232035821 |
| chr1 | 244118283 | 244118333 |
| chr1 | 244188960 | 244189010 |
| chr1 | 244189592 | 244189642 |
| chr1 | 245302959 | 245303009 |
| chr1 | 245304283 | 245304333 |
| chr20 | 10057789 | 10057839 |
| chr20 | 19992867 | 19992917 |
| chr20 | 20305584 | 20305634 |
| chr20 | 22418314 | 22418364 |
| chr20 | 32043191 | 32043241 |
| chr20 | 43685997 | 43686047 |
| chr20 | 51331092 | 51331142 |
| chr20 | 57341955 | 57342005 |
| chr20 | 58051857 | 58051907 |
| chr20 | 61836318 | 61836368 |
| chr20 | 62779058 | 62779108 |
| chr21 | 33587691 | 33587741 |
| chr21 | 33857170 | 33857220 |
| chr21 | 43020296 | 43020346 |
| chr21 | 44202459 | 44202509 |
| chr22 | 31806060 | 31806110 |
| chr22 | 37293451 | 37293501 |
| chr22 | 38825374 | 38825424 |
| chrX | 8991717 | 8991767 |
| chrX | 12811361 | 12811411 |
| chrX | 19376905 | 19376955 |
| chrX | 30213598 | 30213648 |
| chrX | 46443277 | 46443327 |
| chrX | 61272251 | 61272301 |
| chr2 | 5098616 | 5098666 |
| chr2 | 9374736 | 9374786 |
| chr2 | 20480446 | 20480496 |
| chr2 | 34046124 | 34046174 |
| chr2 | 42735645 | 42735695 |
| chr2 | 113341496 | 113341546 |
| chr2 | 119974817 | 119974867 |
| chr2 | 179175503 | 179175553 |
| chr2 | 179296325 | 179296375 |
| chr2 | 206942590 | 206942640 |
| chr2 | 221552542 | 221552592 |
| chr11 | 46128612 | 46128662 |
| chr2 | 242872942 | 242872992 |
| chr3 | 9682131 | 9682181 |
| chr3 | 15899609 | 15899659 |
| chr3 | 23681342 | 23681392 |
| chr3 | 32760896 | 32760946 |
| chr3 | 38322097 | 38322147 |
| chr3 | 47274267 | 47274317 |
| chr3 | 59553708 | 59553758 |
| chr3 | 61675275 | 61675325 |
| chr3 | 70655463 | 70655513 |
| chr3 | 96695487 | 96695537 |
| chr3 | 129047683 | 129047733 |
| chr4 | 2339095 | 2339145 |
| chr4 | 2976612 | 2976662 |
| chr4 | 25058244 | 25058294 |
| chr4 | 37755268 | 37755318 |
| chr4 | 39629975 | 39630025 |
| chr4 | 78398025 | 78398075 |
| chr4 | 95830643 | 95830693 |
| chr4 | 109983158 | 109983208 |
| chr4 | 109986196 | 109986246 |
| chr4 | 171353331 | 171353381 |
| chr4 | 186989204 | 186989254 |
| chr4 | 186989358 | 186989408 |
| chr5 | 1338823 | 1338873 |
| chr5 | 6803102 | 6803152 |
| chr5 | 11895748 | 11895798 |
| chr5 | 35231519 | 35231569 |
| chr5 | 35231411 | 35231461 |
| chr5 | 61747211 | 61747261 |
| chr11 | 70677655 | 70677705 |
| chr5 | 112013103 | 112013153 |
| chr5 | 116285889 | 116285939 |
| chr5 | 141004505 | 141004555 |
| chr5 | 170999066 | 170999116 |
| chr5 | 170998900 | 170998950 |
| chr5 | 176983578 | 176983628 |
| chr5 | 179070738 | 179070788 |
| chr5 | 179100313 | 179100363 |
| chr5 | 179101017 | 179101067 |
| chr6 | 27218344 | 27218394 |
| chr6 | 34434503 | 34434553 |
| chr6 | 42547789 | 42547839 |
| chr6 | 47763286 | 47763336 |
| chr6 | 47765624 | 47765674 |
| chr6 | 53230603 | 53230653 |
| chr6 | 56971248 | 56971298 |
| chr6 | 57636880 | 57636930 |
| chr11 | 90365723 | 90365773 |
| chr6 | 107319257 | 107319307 |
| chr6 | 109830171 | 109830221 |
| chr6 | 150864813 | 150864863 |
| chr6 | 152334351 | 152334401 |
| chr6 | 168133865 | 168133915 |
| chr6 | 168134180 | 168134230 |
| chr7 | 1065164 | 1065214 |
| chr7 | 1070106 | 1070156 |
| chr7 | 1099167 | 1099217 |
| chr7 | 2269189 | 2269239 |
| chr7 | 5360397 | 5360447 |
| chr7 | 6369076 | 6369126 |
| chr7 | 6368935 | 6368985 |
| chr7 | 24786849 | 24786899 |
| chr11 | 113158586 | 113158636 |
| chr7 | 72511889 | 72511939 |
| chr11 | 116353621 | 116353671 |
| chr7 | 85815185 | 85815235 |
| chr11 | 117360884 | 117360934 |
| chr7 | 154374651 | 154374701 |
| chr8 | 28960483 | 28960533 |
| chr8 | 40967944 | 40967994 |
| chr8 | 95408477 | 95408527 |
| chr8 | 98652616 | 98652666 |
| chr8 | 144526899 | 144526949 |
| chr9 | 67110222 | 67110272 |
| chr9 | 72670216 | 72670266 |
| chr9 | 74554257 | 74554307 |
| chr9 | 87558359 | 87558409 |
| chr9 | 90202117 | 90202167 |
| chr9 | 93087507 | 93087557 |
| chr9 | 96128491 | 96128541 |
| chr9 | 110093480 | 110093530 |
| chr9 | 121551889 | 121551939 |
| chr12 | 2781765 | 2781815 |
| chr12 | 2781843 | 2781893 |
| chr9 | 135151101 | 135151151 |
| chr9 | 135151024 | 135151074 |
| chr12 | 3347814 | 3347864 |
| chr10 | 22284746 | 22284796 |
| chr12 | 7985836 | 7985886 |
| chr10 | 1124969 | 1125019 |
| chr12 | 60621418 | 60621468 |
| chr12 | 93634965 | 93635015 |
| chr12 | 94366509 | 94366559 |
| chr10 | 30009840 | 30009890 |
| chr10 | 30011488 | 30011538 |
| chr12 | 105339709 | 105339759 |
| chr10 | 30967088 | 30967138 |
| chr12 | 112148798 | 112148848 |
| chr10 | 32093904 | 32093954 |
| chr12 | 120341567 | 120341617 |
| chr12 | 123566612 | 123566662 |
| chr12 | 123856981 | 123857031 |
| chr12 | 128982028 | 128982078 |
| chr12 | 129496898 | 129496948 |
| chr12 | 130915804 | 130915854 |
| chr12 | 131392043 | 131392093 |
| chr12 | 131526761 | 131526811 |
| chr13 | 18066515 | 18066565 |
| chr13 | 41396370 | 41396420 |
| chr13 | 63298021 | 63298071 |
| chr13 | 73619515 | 73619565 |
| chr10 | 43208286 | 43208336 |
| chr13 | 110568257 | 110568307 |
| chr13 | 111059618 | 111059668 |
| chr14 | 21927814 | 21927864 |
| chr14 | 22682492 | 22682542 |
| chr14 | 56870264 | 56870314 |
| chr14 | 67658335 | 67658385 |
| chr14 | 77582438 | 77582488 |
| chr14 | 77588567 | 77588617 |
| chr10 | 53750480 | 53750530 |
| chr15 | 20515997 | 20516047 |
| chr15 | 20515844 | 20515894 |
| chr10 | 64031986 | 64032036 |
| chr15 | 42430182 | 42430232 |
| chr15 | 42666358 | 42666408 |
| chr15 | 48366584 | 48366634 |
| chr15 | 56874744 | 56874794 |
| chr15 | 61329848 | 61329898 |
| chr15 | 64522138 | 64522188 |
| chr15 | 67482100 | 67482150 |
| chr15 | 71546171 | 71546221 |
| chr10 | 69227420 | 69227470 |
| chr10 | 69227592 | 69227642 |
| chr10 | 69228407 | 69228457 |
| chr15 | 87498449 | 87498499 |
| chr15 | 90249504 | 90249554 |
| chr15 | 91481977 | 91482027 |
| chr15 | 91481560 | 91481610 |
| chr16 | 1016123 | 1016173 |
| chr16 | 1435160 | 1435210 |
| chr10 | 3072381 | 3072431 |
| chr16 | 17453812 | 17453862 |
| chr16 | 21143401 | 21143451 |
| chr16 | 24626465 | 24626515 |
| chr16 | 29689378 | 29689428 |
| chr16 | 51813218 | 51813268 |
| chr16 | 75011723 | 75011773 |
| chr16 | 87250716 | 87250766 |
| chr10 | 75279354 | 75279404 |
| chr17 | 4612399 | 4612449 |
| chr17 | 10818486 | 10818536 |
| chr17 | 21290770 | 21290820 |
| chr17 | 26386919 | 26386969 |
| chr17 | 30826213 | 30826263 |

Table 15H. Repeat Category Cheshire

| | | |
|---|---|---|
| chr10 | 81773010 | 81773060 |
| chr17 | 74394540 | 74394590 |
| chr17 | 74398080 | 74398130 |
| chr18 | 11712490 | 11712540 |
| chr1 | 10618225 | 10618275 |
| chr1 | 88816567 | 88816617 |
| chr1 | 88818538 | 88818588 |
| chr1 | 190423949 | 190423999 |
| chr1 | 225761246 | 225761296 |
| chr20 | 51228228 | 51228278 |
| chr22 | 17998917 | 17998967 |
| chr22 | 17998810 | 17998860 |
| chr22 | 19586488 | 19586538 |
| chrX | 72684888 | 72684938 |
| chrX | 113389993 | 113390043 |
| chr2 | 28964171 | 28964221 |
| chr11 | 23114011 | 23114061 |
| chr2 | 109071884 | 109071934 |
| chr2 | 122577295 | 122577345 |
| chr2 | 172183233 | 172183283 |
| chr2 | 172183143 | 172183193 |
| chr3 | 50749233 | 50749283 |
| chr3 | 87061966 | 87062016 |
| chr3 | 87068970 | 87069020 |
| chr3 | 102750524 | 102750574 |
| chr3 | 102750585 | 102750635 |
| chr4 | 129426576 | 129426626 |
| chr4 | 129428464 | 129428514 |
| chr4 | 152608732 | 152608782 |
| chr5 | 139351517 | 139351567 |
| chr6 | 20915720 | 20915770 |
| chr6 | 33485369 | 33485419 |
| chr6 | 56888105 | 56888155 |
| chr6 | 99364715 | 99364765 |
| chr6 | 99364581 | 99364631 |
| chr7 | 20492031 | 20492081 |
| chr7 | 20494483 | 20494533 |
| chr7 | 80836915 | 80836965 |
| chr7 | 105837011 | 105837061 |
| chr7 | 133710595 | 133710645 |
| chr8 | 2391172 | 2391222 |
| chr8 | 6190827 | 6190877 |
| chr8 | 17096057 | 17096107 |
| chr8 | 17095816 | 17095866 |
| chr8 | 145790841 | 145790891 |
| chr8 | 145793097 | 145793147 |
| chr9 | 33834702 | 33834752 |
| chr9 | 33836330 | 33836380 |
| chr9 | 35114287 | 35114337 |
| chr9 | 88187250 | 88187300 |
| chr9 | 135746796 | 135746846 |
| chr9 | 135757128 | 135757178 |
| chr12 | 43169291 | 43169341 |
| chr13 | 18467805 | 18467855 |
| chr13 | 18467478 | 18467528 |
| chr10 | 35545179 | 35545229 |
| chr10 | 35547095 | 35547145 |
| chr10 | 45423258 | 45423308 |
| chr14 | 24983410 | 24983460 |
| chr14 | 59429196 | 59429246 |
| chr14 | 65127347 | 65127397 |
| chr14 | 103871137 | 103871187 |
| chr15 | 43363263 | 43363313 |
| chr16 | 67925483 | 67925533 |
| chr10 | 75406089 | 75406139 |
| chr17 | 6912483 | 6912533 |

Table 15I. Repeat Category ERVL

| | | |
|---|---|---|
| chr17 | 39094931 | 39094981 |
| chr17 | 39369869 | 39369919 |
| chr17 | 39516492 | 39516542 |
| chr17 | 39516321 | 39516371 |
| chr17 | 39759047 | 39759097 |
| chr17 | 40282366 | 40282416 |
| chr17 | 40633403 | 40633453 |
| chr17 | 41664501 | 41664551 |
| chr10 | 81979509 | 81979559 |
| chr17 | 41916256 | 41916306 |
| chr17 | 42159421 | 42159471 |
| chr17 | 42324152 | 42324202 |
| chr17 | 42525744 | 42525794 |
| chr17 | 42575356 | 42575406 |
| chr17 | 42576034 | 42576084 |
| chr17 | 42847498 | 42847548 |
| chr17 | 42852159 | 42852209 |
| chr17 | 42892627 | 42892677 |
| chr17 | 42940446 | 42940496 |
| chr17 | 42940779 | 42940829 |
| chr10 | 82175552 | 82175602 |
| chr17 | 43505918 | 43505968 |
| chr17 | 43596128 | 43596178 |
| chr17 | 43941931 | 43941981 |
| chr17 | 44145331 | 44145381 |
| chr17 | 44184415 | 44184465 |
| chr17 | 44184897 | 44184947 |
| chr17 | 46176066 | 46176116 |
| chr17 | 47210829 | 47210879 |
| chr17 | 47213443 | 47213493 |
| chr17 | 47215152 | 47215202 |
| chr10 | 83657121 | 83657171 |
| chr17 | 47641123 | 47641173 |
| chr17 | 47825644 | 47825694 |
| chr17 | 47826731 | 47826781 |
| chr17 | 47828106 | 47828156 |
| chr17 | 47829837 | 47829887 |
| chr17 | 47831363 | 47831413 |
| chr17 | 48871203 | 48871253 |
| chr17 | 49028352 | 49028402 |
| chr17 | 49029549 | 49029599 |
| chr17 | 49429152 | 49429202 |
| chr17 | 49841124 | 49841174 |
| chr17 | 49888862 | 49888912 |
| chr17 | 50432924 | 50432974 |
| chr17 | 51513647 | 51513697 |
| chr17 | 51756645 | 51756695 |
| chr17 | 53176089 | 53176139 |
| chr17 | 53720827 | 53720877 |
| chr17 | 53879889 | 53879939 |
| chr17 | 53879692 | 53879742 |
| chr17 | 54435998 | 54436048 |
| chr17 | 55559659 | 55559709 |
| chr17 | 55563965 | 55564015 |
| chr10 | 85805403 | 85805453 |
| chr17 | 57266734 | 57266784 |
| chr10 | 85805945 | 85805995 |
| chr17 | 58021035 | 58021085 |
| chr17 | 58056575 | 58056625 |
| chr10 | 85978216 | 85978266 |
| chr10 | 85989986 | 85990036 |
| chr17 | 59112058 | 59112108 |
| chr17 | 59371761 | 59371811 |
| chr17 | 60734758 | 60734808 |
| chr17 | 60736715 | 60736765 |
| chr17 | 61446962 | 61447012 |
| chr17 | 61506008 | 61506058 |
| chr17 | 61775297 | 61775347 |
| chr10 | 86898106 | 86898156 |
| chr17 | 62723221 | 62723271 |
| chr17 | 63264844 | 63264894 |
| chr17 | 63774960 | 63775010 |
| chr17 | 64006055 | 64006105 |
| chr17 | 64813207 | 64813257 |
| chr17 | 65872706 | 65872756 |
| chr10 | 87981819 | 87981869 |
| chr17 | 66557546 | 66557596 |
| chr17 | 66575703 | 66575753 |
| chr17 | 67161361 | 67161411 |
| chr17 | 67353834 | 67353884 |
| chr17 | 67399645 | 67399695 |
| chr17 | 68455881 | 68455931 |
| chr17 | 70735858 | 70735908 |
| chr17 | 71139590 | 71139640 |
| chr17 | 71270125 | 71270175 |
| chr10 | 88270016 | 88270066 |
| chr17 | 72338395 | 72338445 |
| chr17 | 72338191 | 72338241 |
| chr17 | 72581475 | 72581525 |
| chr10 | 88437821 | 88437871 |
| chr17 | 72818570 | 72818620 |
| chr17 | 73095204 | 73095254 |
| chr17 | 73315559 | 73315609 |
| chr17 | 73323336 | 73323386 |
| chr17 | 73323158 | 73323208 |
| chr17 | 73545559 | 73545609 |
| chr17 | 73545880 | 73545930 |
| chr17 | 74455813 | 74455863 |
| chr17 | 75055767 | 75055817 |
| chr10 | 4254262 | 4254312 |
| chr17 | 75182551 | 75182601 |
| chr17 | 75380250 | 75380300 |
| chr10 | 4254201 | 4254251 |
| chr17 | 75474415 | 75474465 |
| chr17 | 75474243 | 75474293 |
| chr17 | 75886905 | 75886955 |
| chr17 | 76057738 | 76057788 |
| chr17 | 76114760 | 76114810 |
| chr17 | 76116883 | 76116933 |
| chr17 | 76150864 | 76150914 |
| chr17 | 76331227 | 76331277 |
| chr17 | 76704181 | 76704231 |
| chr17 | 77124828 | 77124878 |
| chr17 | 77203156 | 77203206 |
| chr17 | 77209544 | 77209594 |
| chr17 | 77209288 | 77209338 |
| chr17 | 77210395 | 77210445 |
| chr17 | 77260496 | 77260546 |
| chr17 | 77261469 | 77261519 |
| chr17 | 77557056 | 77557106 |
| chr17 | 77583919 | 77583969 |
| chr17 | 77718381 | 77718431 |
| chr17 | 77797523 | 77797573 |
| chr17 | 77842919 | 77842969 |
| chr17 | 77959810 | 77959860 |
| chr17 | 77959673 | 77959723 |
| chr17 | 78443863 | 78443913 |
| chr10 | 91253781 | 91253831 |
| chr10 | 91253714 | 91253764 |
| chr17 | 78777646 | 78777696 |
| chr17 | 79277553 | 79277603 |
| chr17 | 79494069 | 79494119 |
| chr17 | 79499876 | 79499926 |
| chr10 | 92255465 | 92255515 |
| chr17 | 79663446 | 79663496 |
| chr10 | 92329690 | 92329740 |
| chr10 | 92329554 | 92329604 |
| chr10 | 92585098 | 92585148 |
| chr17 | 80962427 | 80962477 |
| chr17 | 81032196 | 81032246 |
| chr17 | 81655590 | 81655640 |
| chr17 | 81656186 | 81656236 |
| chr18 | 844215 | 844265 |
| chr18 | 869566 | 869616 |
| chr18 | 993795 | 993845 |
| chr18 | 2179898 | 2179948 |
| chr18 | 2396055 | 2396105 |
| chr18 | 2472063 | 2472113 |
| chr18 | 2540846 | 2540896 |
| chr18 | 2823661 | 2823711 |
| chr18 | 3152709 | 3152759 |
| chr18 | 3619827 | 3619877 |
| chr18 | 4928111 | 4928161 |
| chr18 | 5008719 | 5008769 |
| chr18 | 5009836 | 5009886 |
| chr10 | 93836012 | 93836062 |
| chr18 | 5868454 | 5868504 |
| chr18 | 7262926 | 7262976 |
| chr18 | 7366186 | 7366236 |
| chr18 | 7475658 | 7475708 |
| chr10 | 4757976 | 4758026 |
| chr18 | 8527528 | 8527578 |
| chr18 | 8561044 | 8561094 |
| chr18 | 8571507 | 8571557 |
| chr18 | 8952863 | 8952913 |
| chr18 | 8952555 | 8952605 |
| chr18 | 9748034 | 9748084 |
| chr18 | 10893257 | 10893307 |
| chr18 | 11668056 | 11668106 |
| chr18 | 11714572 | 11714622 |
| chr18 | 11935591 | 11935641 |
| chr18 | 12757099 | 12757149 |
| chr18 | 12885078 | 12885128 |
| chr18 | 12891931 | 12891981 |
| chr18 | 12932204 | 12932254 |
| chr18 | 13880948 | 13880998 |
| chr18 | 15113896 | 15113946 |
| chr18 | 16781020 | 16781070 |
| chr18 | 16993075 | 16993125 |
| chr18 | 17463654 | 17463704 |
| chr18 | 17540949 | 17540999 |
| chr18 | 17840531 | 17840581 |
| chr10 | 95055901 | 95055951 |
| chr18 | 19097151 | 19097201 |
| chr10 | 95264677 | 95264727 |
| chr18 | 19479651 | 19479701 |
| chr18 | 19530090 | 19530140 |
| chr18 | 20271821 | 20271871 |
| chr18 | 20463819 | 20463869 |
| chr18 | 20463699 | 20463749 |
| chr18 | 21560382 | 21560432 |
| chr18 | 22003891 | 22003941 |
| chr18 | 22041995 | 22042045 |
| chr18 | 22204999 | 22205049 |
| chr18 | 23042636 | 23042686 |
| chr18 | 24280020 | 24280070 |
| chr10 | 95792518 | 95792568 |
| chr18 | 26375253 | 26375303 |
| chr18 | 26386186 | 26386236 |
| chr18 | 26785077 | 26785127 |
| chr18 | 27630945 | 27630995 |
| chr18 | 27998944 | 27998994 |
| chr18 | 28343576 | 28343626 |
| chr18 | 28462180 | 28462230 |
| chr18 | 29034028 | 29034078 |
| chr18 | 29175938 | 29175988 |
| chr18 | 29256871 | 29256921 |
| chr18 | 30087080 | 30087130 |
| chr18 | 30289510 | 30289560 |
| chr18 | 31133949 | 31133999 |
| chr18 | 31506619 | 31506669 |
| chr18 | 31538141 | 31538191 |
| chr18 | 31938159 | 31938209 |
| chr18 | 32534916 | 32534966 |
| chr18 | 33712437 | 33712487 |
| chr18 | 34500290 | 34500340 |
| chr18 | 35294094 | 35294144 |
| chr18 | 35417984 | 35418034 |
| chr18 | 35971945 | 35971995 |
| chr18 | 36098054 | 36098104 |
| chr18 | 36221106 | 36221156 |
| chr18 | 37083225 | 37083275 |
| chr18 | 38016939 | 38016989 |
| chr18 | 38016466 | 38016516 |
| chr18 | 39314666 | 39314716 |
| chr18 | 39331130 | 39331180 |
| chr18 | 39330976 | 39331026 |
| chr18 | 41814726 | 41814776 |
| chr18 | 42490825 | 42490875 |
| chr18 | 42958118 | 42958168 |
| chr18 | 43598178 | 43598228 |
| chr18 | 43746242 | 43746292 |
| chr10 | 97156074 | 97156124 |
| chr10 | 5129359 | 5129409 |
| chr18 | 46080667 | 46080717 |
| chr18 | 46582105 | 46582155 |
| chr18 | 46624091 | 46624141 |
| chr18 | 47025108 | 47025158 |
| chr18 | 47174523 | 47174573 |
| chr18 | 47323488 | 47323538 |
| chr18 | 47960164 | 47960214 |
| chr18 | 47961346 | 47961396 |
| chr10 | 97524859 | 97524909 |
| chr18 | 50286093 | 50286143 |
| chr18 | 50682499 | 50682549 |
| chr18 | 51448579 | 51448629 |
| chr10 | 97590744 | 97590794 |
| chr18 | 52459278 | 52459328 |
| chr18 | 53442586 | 53442636 |
| chr18 | 54623387 | 54623437 |
| chr18 | 54637512 | 54637562 |
| chr10 | 97933975 | 97934025 |
| chr18 | 57837183 | 57837233 |
| chr18 | 58254726 | 58254776 |
| chr18 | 58891377 | 58891427 |
| chr10 | 98189574 | 98189624 |
| chr18 | 59263778 | 59263828 |
| chr18 | 60093475 | 60093525 |
| chr18 | 60103301 | 60103351 |
| chr10 | 98244194 | 98244244 |
| chr18 | 60959160 | 60959210 |
| chr18 | 62588138 | 62588188 |
| chr18 | 62588638 | 62588688 |
| chr18 | 62808467 | 62808517 |
| chr18 | 63005517 | 63005567 |
| chr18 | 63421950 | 63422000 |
| chr18 | 63999808 | 63999858 |
| chr18 | 65251323 | 65251373 |
| chr18 | 66218240 | 66218290 |
| chr18 | 67332499 | 67332549 |
| chr18 | 67370922 | 67370972 |
| chr18 | 67423755 | 67423805 |
| chr18 | 67484894 | 67484944 |
| chr18 | 67930624 | 67930674 |
| chr18 | 68045530 | 68045580 |
| chr18 | 68910954 | 68911004 |
| chr18 | 68944895 | 68944945 |
| chr18 | 69095822 | 69095872 |
| chr18 | 69101334 | 69101384 |
| chr18 | 70341686 | 70341736 |
| chr18 | 71015702 | 71015752 |
| chr18 | 71705141 | 71705191 |
| chr18 | 72008105 | 72008155 |
| chr18 | 72370037 | 72370087 |
| chr18 | 72409546 | 72409596 |
| chr18 | 72409373 | 72409423 |
| chr18 | 72426324 | 72426374 |
| chr18 | 73612095 | 73612145 |
| chr18 | 73830534 | 73830584 |
| chr18 | 73994141 | 73994191 |
| chr18 | 73994198 | 73994248 |
| chr18 | 74012476 | 74012526 |
| chr18 | 74481918 | 74481968 |
| chr18 | 74923662 | 74923712 |
| chr10 | 99121087 | 99121137 |
| chr18 | 75730292 | 75730342 |
| chr18 | 75775885 | 75775935 |
| chr18 | 75923806 | 75923856 |
| chr19 | 299766 | 299816 |
| chr19 | 303322 | 303372 |
| chr19 | 1842578 | 1842628 |
| chr19 | 1878847 | 1878897 |
| chr19 | 2075531 | 2075581 |
| chr19 | 2390641 | 2390691 |
| chr19 | 2419597 | 2419647 |
| chr10 | 100731341 | 100731391 |
| chr19 | 2685558 | 2685608 |
| chr19 | 2713088 | 2713138 |
| chr19 | 2748064 | 2748114 |
| chr19 | 2799091 | 2799141 |
| chr19 | 2844874 | 2844924 |
| chr19 | 3150092 | 3150142 |
| chr19 | 3281128 | 3281178 |
| chr10 | 100996815 | 100996865 |
| chr19 | 3517224 | 3517274 |
| chr10 | 101028939 | 101028989 |
| chr19 | 3642214 | 3642264 |
| chr19 | 4209367 | 4209417 |
| chr10 | 101552627 | 101552677 |
| chr19 | 5404904 | 5404954 |
| chr19 | 5913243 | 5913293 |
| chr19 | 6334424 | 6334474 |
| chr19 | 6440485 | 6440535 |
| chr19 | 6508751 | 6508801 |
| chr19 | 6539625 | 6539675 |
| chr19 | 6744389 | 6744439 |
| chr19 | 6821738 | 6821788 |
| chr19 | 7015437 | 7015487 |
| chr19 | 7039798 | 7039848 |
| chr19 | 7044500 | 7044550 |
| chr19 | 7714139 | 7714189 |
| chr19 | 8673484 | 8673534 |
| chr19 | 9112316 | 9112366 |
| chr19 | 9112094 | 9112144 |
| chr19 | 9179990 | 9180040 |
| chr19 | 9217326 | 9217376 |
| chr19 | 9395354 | 9395404 |
| chr19 | 9569772 | 9569822 |
| chr19 | 9703332 | 9703382 |
| chr19 | 9710880 | 9710930 |
| chr10 | 102280924 | 102280974 |
| chr19 | 10214215 | 10214265 |
| chr19 | 10429779 | 10429829 |
| chr19 | 10800877 | 10800927 |
| chr19 | 11019425 | 11019475 |
| chr19 | 11286591 | 11286641 |
| chr19 | 11673644 | 11673694 |
| chr19 | 11748558 | 11748608 |
| chr19 | 11913797 | 11913847 |
| chr19 | 12045258 | 12045308 |
| chr19 | 12058125 | 12058175 |
| chr19 | 12095957 | 12096007 |
| chr19 | 12109500 | 12109550 |
| chr19 | 12144845 | 12144895 |
| chr19 | 12173944 | 12173994 |
| chr19 | 12229801 | 12229851 |
| chr19 | 12232261 | 12232311 |
| chr19 | 12254092 | 12254142 |
| chr19 | 12311024 | 12311074 |
| chr19 | 12457118 | 12457168 |
| chr19 | 12489569 | 12489619 |
| chr19 | 12582109 | 12582159 |
| chr19 | 12605088 | 12605138 |
| chr19 | 12944228 | 12944278 |
| chr19 | 13190102 | 13190152 |
| chr19 | 13191417 | 13191467 |
| chr19 | 13368642 | 13368692 |
| chr19 | 13454484 | 13454534 |
| chr19 | 13636202 | 13636252 |
| chr19 | 14020160 | 14020210 |
| chr19 | 14040446 | 14040496 |
| chr19 | 14149532 | 14149582 |
| chr19 | 14281046 | 14281096 |
| chr19 | 14282468 | 14282518 |
| chr19 | 14296119 | 14296169 |
| chr19 | 14317208 | 14317258 |
| chr19 | 14317012 | 14317062 |
| chr19 | 14997795 | 14997845 |
| chr19 | 15474540 | 15474590 |
| chr19 | 15497122 | 15497172 |
| chr19 | 15742764 | 15742814 |
| chr19 | 16657279 | 16657329 |
| chr19 | 16695896 | 16695946 |
| chr19 | 17155693 | 17155743 |
| chr19 | 17618962 | 17619012 |
| chr19 | 17707258 | 17707308 |
| chr19 | 18385006 | 18385056 |
| chr19 | 18793111 | 18793161 |
| chr19 | 19087829 | 19087879 |
| chr19 | 19161133 | 19161183 |
| chr19 | 19675509 | 19675559 |
| chr19 | 19734668 | 19734718 |
| chr19 | 19735950 | 19736000 |
| chr19 | 19834257 | 19834307 |
| chr19 | 20023775 | 20023825 |
| chr19 | 20038058 | 20038108 |
| chr19 | 20041410 | 20041460 |
| chr19 | 20044959 | 20045009 |
| chr19 | 20125977 | 20126027 |
| chr19 | 23318648 | 23318698 |
| chr19 | 23816226 | 23816276 |
| chr19 | 33588630 | 33588680 |
| chr19 | 34612361 | 34612411 |
| chr19 | 34637012 | 34637062 |
| chr19 | 35278791 | 35278841 |
| chr19 | 35486751 | 35486801 |
| chr10 | 5717805 | 5717855 |
| chr19 | 36206063 | 36206113 |
| chr19 | 37733028 | 37733078 |
| chr19 | 38095310 | 38095360 |
| chr19 | 38213021 | 38213071 |
| chr19 | 38367114 | 38367164 |
| chr19 | 38925846 | 38925896 |
| chr19 | 38948511 | 38948561 |
| chr19 | 39014444 | 39014494 |
| chr19 | 39419557 | 39419607 |
| chr19 | 40350048 | 40350098 |
| chr19 | 40349850 | 40349900 |
| chr19 | 40362884 | 40362934 |
| chr19 | 40580153 | 40580203 |
| chr19 | 41182352 | 41182402 |
| chr19 | 41344828 | 41344878 |
| chr19 | 41346425 | 41346475 |
| chr19 | 41347917 | 41347967 |
| chr19 | 41350369 | 41350419 |
| chr19 | 41622630 | 41622680 |
| chr19 | 41819158 | 41819208 |
| chr19 | 42029330 | 42029380 |
| chr19 | 42188319 | 42188369 |
| chr19 | 42221008 | 42221058 |
| chr19 | 42586591 | 42586641 |
| chr19 | 42927372 | 42927422 |
| chr19 | 42995268 | 42995318 |
| chr19 | 43476713 | 43476763 |
| chr19 | 43478244 | 43478294 |
| chr10 | 104889876 | 104889926 |
| chr19 | 44129557 | 44129607 |
| chr19 | 44144506 | 44144556 |
| chr19 | 44144760 | 44144810 |
| chr19 | 44185437 | 44185487 |
| chr19 | 44185519 | 44185569 |
| chr19 | 44194994 | 44195044 |
| chr19 | 44195135 | 44195185 |
| chr19 | 44195599 | 44195649 |
| chr19 | 44196596 | 44196646 |
| chr19 | 44199316 | 44199366 |
| chr19 | 44200272 | 44200322 |
| chr19 | 44206013 | 44206063 |
| chr19 | 44459047 | 44459097 |
| chr19 | 44581466 | 44581516 |
| chr19 | 44706594 | 44706644 |
| chr19 | 44707046 | 44707096 |
| chr19 | 44707125 | 44707175 |
| chr19 | 44708077 | 44708127 |
| chr19 | 44715856 | 44715906 |
| chr19 | 45408107 | 45408157 |
| chr10 | 105303437 | 105303487 |
| chr10 | 105303232 | 105303282 |
| chr10 | 105492007 | 105492057 |
| chr19 | 46028477 | 46028527 |
| chr19 | 46353047 | 46353097 |
| chr19 | 46356963 | 46357013 |
| chr19 | 46447598 | 46447648 |
| chr19 | 46631449 | 46631499 |
| chr19 | 46651352 | 46651402 |
| chr19 | 47224570 | 47224620 |
| chr19 | 47728580 | 47728630 |
| chr19 | 47729535 | 47729585 |
| chr19 | 47848009 | 47848059 |
| chr10 | 106643402 | 106643452 |
| chr10 | 5902357 | 5902407 |
| chr19 | 48540321 | 48540371 |
| chr10 | 106907327 | 106907377 |
| chr19 | 48561987 | 48562037 |
| chr19 | 48576172 | 48576222 |
| chr19 | 48576229 | 48576279 |
| chr10 | 107159974 | 107160024 |
| chr19 | 49213673 | 49213723 |
| chr19 | 49301081 | 49301131 |
| chr19 | 49300603 | 49300653 |
| chr19 | 49300938 | 49300988 |
| chr19 | 49306890 | 49306940 |
| chr19 | 49307169 | 49307219 |
| chr19 | 49378572 | 49378622 |
| chr19 | 49380264 | 49380314 |
| chr19 | 49576488 | 49576538 |
| chr19 | 49669421 | 49669471 |
| chr19 | 50475594 | 50475644 |
| chr19 | 50521631 | 50521681 |
| chr19 | 50739219 | 50739269 |
| chr19 | 50742141 | 50742191 |
| chr19 | 50742222 | 50742272 |
| chr19 | 50802406 | 50802456 |
| chr19 | 51102865 | 51102915 |
| chr19 | 51103074 | 51103124 |
| chr19 | 51103794 | 51103844 |
| chr19 | 51405439 | 51405489 |
| chr19 | 51405283 | 51405333 |
| chr19 | 51528763 | 51528813 |
| chr19 | 51564307 | 51564357 |
| chr19 | 51586544 | 51586594 |
| chr19 | 51586788 | 51586838 |
| chr19 | 51586908 | 51586958 |
| chr19 | 51587084 | 51587134 |
| chr19 | 51587374 | 51587424 |
| chr10 | 109827707 | 109827757 |
| chr19 | 51626705 | 51626755 |
| chr19 | 51626910 | 51626960 |
| chr19 | 51627212 | 51627262 |
| chr19 | 51627573 | 51627623 |
| chr19 | 51685203 | 51685253 |
| chr19 | 51904744 | 51904794 |
| chr19 | 51904905 | 51904955 |
| chr19 | 51905561 | 51905611 |
| chr19 | 51905698 | 51905748 |
| chr19 | 51905882 | 51905932 |
| chr10 | 110770595 | 110770645 |
| chr19 | 52459396 | 52459446 |
| chr19 | 52459695 | 52459745 |
| chr19 | 52490463 | 52490513 |
| chr19 | 52607415 | 52607465 |
| chr19 | 52610298 | 52610348 |
| chr19 | 52619852 | 52619902 |
| chr19 | 53094631 | 53094681 |
| chr19 | 53169290 | 53169340 |
| chr19 | 53255201 | 53255251 |
| chr19 | 53265155 | 53265205 |
| chr19 | 53462387 | 53462437 |
| chr19 | 53784377 | 53784427 |
| chr19 | 53979613 | 53979663 |
| chr19 | 54409083 | 54409133 |
| chr19 | 54422177 | 54422227 |
| chr19 | 54476385 | 54476435 |
| chr19 | 54480619 | 54480669 |
| chr19 | 54705615 | 54705665 |
| chr10 | 5993640 | 5993690 |
| chr19 | 54912714 | 54912764 |
| chr19 | 54927371 | 54927421 |
| chr19 | 54929211 | 54929261 |
| chr19 | 54930604 | 54930654 |
| chr19 | 55108840 | 55108890 |
| chr19 | 55143128 | 55143178 |
| chr19 | 55201725 | 55201775 |
| chr19 | 55606466 | 55606516 |
| chr19 | 55850247 | 55850297 |
| chr19 | 55868409 | 55868459 |
| chr19 | 56452878 | 56452928 |
| chr19 | 56452667 | 56452717 |
| chr19 | 56495661 | 56495711 |
| chr10 | 112819591 | 112819641 |
| chr10 | 112854925 | 112854975 |
| chr10 | 113008190 | 113008240 |
| chr19 | 56994206 | 56994256 |
| chr19 | 56996534 | 56996584 |
| chr19 | 57106145 | 57106195 |
| chr19 | 57168350 | 57168400 |
| chr19 | 57443595 | 57443645 |
| chr19 | 57583805 | 57583855 |
| chr19 | 58225411 | 58225461 |
| chr19 | 58226657 | 58226707 |
| chr19 | 58489057 | 58489107 |
| chr19 | 58519422 | 58519472 |
| chr19 | 58699637 | 58699687 |
| chr19 | 58701580 | 58701630 |
| chr19 | 58814256 | 58814306 |
| chr19 | 59238897 | 59238947 |
| chr19 | 59274379 | 59274429 |
| chr19 | 59427637 | 59427687 |
| chr19 | 59443366 | 59443416 |
| chr19 | 59498707 | 59498757 |
| chr19 | 59499175 | 59499225 |
| chr19 | 59498636 | 59498686 |
| chr19 | 59572389 | 59572439 |
| chr19 | 59648218 | 59648268 |
| chr19 | 59865246 | 59865296 |
| chr19 | 59889305 | 59889355 |
| chr19 | 60008278 | 60008328 |
| chr19 | 60415782 | 60415832 |
| chr19 | 60418163 | 60418213 |
| chr19 | 60529748 | 60529798 |
| chr19 | 60674467 | 60674517 |
| chr19 | 60931278 | 60931328 |
| chr19 | 61583607 | 61583657 |
| chr19 | 61733902 | 61733952 |
| chr19 | 61855423 | 61855473 |
| chr19 | 62154717 | 62154767 |
| chr19 | 62470832 | 62470882 |
| chr19 | 62542156 | 62542206 |
| chr19 | 62587080 | 62587130 |
| chr19 | 62763034 | 62763084 |
| chr19 | 62762914 | 62762964 |
| chr19 | 62908476 | 62908526 |
| chr19 | 62979314 | 62979364 |
| chr19 | 63033640 | 63033690 |
| chr19 | 63033496 | 63033546 |
| chr19 | 63035207 | 63035257 |
| chr19 | 63041136 | 63041186 |
| chr19 | 63081082 | 63081132 |
| chr19 | 63090568 | 63090618 |
| chr19 | 63124728 | 63124778 |
| chr1 | 1100839 | 1100889 |
| chr1 | 1101553 | 1101603 |
| chr1 | 1298422 | 1298472 |
| chr1 | 1792237 | 1792287 |
| chr1 | 1959049 | 1959099 |
| chr1 | 1965608 | 1965658 |
| chr1 | 2184528 | 2184578 |
| chr1 | 2703560 | 2703610 |
| chr1 | 2703433 | 2703483 |
| chr1 | 2732525 | 2732575 |
| chr10 | 6163914 | 6163964 |
| chr1 | 3287062 | 3287112 |
| chr1 | 3699449 | 3699499 |
| chr1 | 3699307 | 3699357 |
| chr1 | 3891642 | 3891692 |
| chr1 | 3909422 | 3909472 |
| chr1 | 4082911 | 4082961 |
| chr1 | 4320728 | 4320778 |
| chr1 | 4405286 | 4405336 |
| chr1 | 4406164 | 4406214 |
| chr1 | 4585387 | 4585437 |
| chr1 | 5422370 | 5422420 |
| chr1 | 5422247 | 5422297 |
| chr1 | 5592430 | 5592480 |
| chr1 | 5614386 | 5614436 |
| chr1 | 5750189 | 5750239 |
| chr1 | 6182351 | 6182401 |
| chr1 | 6579051 | 6579101 |
| chr10 | 116227905 | 116227955 |
| chr10 | 116234529 | 116234579 |
| chr1 | 7106875 | 7106925 |
| chr10 | 6193316 | 6193366 |
| chr1 | 7668014 | 7668064 |
| chr1 | 7675012 | 7675062 |
| chr1 | 8045630 | 8045680 |
| chr10 | 116676004 | 116676054 |
| chr1 | 8679520 | 8679570 |
| chr1 | 8706643 | 8706693 |
| chr1 | 8906545 | 8906595 |
| chr1 | 8922053 | 8922103 |
| chr1 | 8922184 | 8922234 |
| chr1 | 9279370 | 9279420 |
| chr1 | 9294515 | 9294565 |
| chr1 | 9297577 | 9297627 |
| chr1 | 9906722 | 9906772 |
| chr1 | 9922438 | 9922488 |
| chr1 | 10690844 | 10690894 |
| chr1 | 10895017 | 10895067 |
| chr1 | 11123075 | 11123125 |
| chr1 | 11148245 | 11148295 |
| chr1 | 11154981 | 11155031 |
| chr10 | 117948122 | 117948172 |
| chr1 | 12273663 | 12273713 |
| chr1 | 13155265 | 13155315 |
| chr1 | 13188928 | 13188978 |
| chr1 | 13374220 | 13374270 |
| chr1 | 14084893 | 14084943 |
| chr1 | 14860266 | 14860316 |
| chr1 | 15202424 | 15202474 |
| chr10 | 118349143 | 118349193 |
| chr1 | 15683769 | 15683819 |
| chr1 | 16495481 | 16495531 |
| chr1 | 16604266 | 16604316 |
| chr1 | 17425323 | 17425373 |
| chr1 | 17425855 | 17425905 |
| chr1 | 17462896 | 17462946 |
| chr1 | 17462836 | 17462886 |
| chr1 | 17660768 | 17660818 |
| chr1 | 17933998 | 17934048 |
| chr1 | 19095012 | 19095062 |
| chr1 | 19332690 | 19332740 |
| chr1 | 19569255 | 19569305 |
| chr1 | 19877240 | 19877290 |
| chr1 | 19877083 | 19877133 |
| chr1 | 20056663 | 20056713 |
| chr1 | 20497029 | 20497079 |
| chr1 | 21001713 | 21001763 |
| chr1 | 21003911 | 21003961 |
| chr1 | 21005323 | 21005373 |
| chr1 | 21217328 | 21217378 |
| chr1 | 22110824 | 22110874 |
| chr1 | 22708140 | 22708190 |
| chr1 | 23270832 | 23270882 |
| chr1 | 23384987 | 23385037 |
| chr10 | 120001081 | 120001131 |
| chr1 | 23626682 | 23626732 |
| chr1 | 23822547 | 23822597 |
| chr1 | 23845639 | 23845689 |
| chr1 | 24101287 | 24101337 |
| chr1 | 24164941 | 24164991 |
| chr1 | 24177735 | 24177785 |
| chr1 | 24549677 | 24549727 |
| chr1 | 24799475 | 24799525 |
| chr1 | 24820455 | 24820505 |
| chr1 | 25520823 | 25520873 |
| chr1 | 25812976 | 25813026 |
| chr1 | 25941896 | 25941946 |
| chr1 | 25980685 | 25980735 |
| chr10 | 120390478 | 120390528 |
| chr10 | 120398639 | 120398689 |
| chr1 | 26741394 | 26741444 |
| chr1 | 26971735 | 26971785 |
| chr1 | 27003012 | 27003062 |
| chr1 | 27021328 | 27021378 |
| chr1 | 27026307 | 27026357 |
| chr1 | 27187885 | 27187935 |
| chr1 | 28673990 | 28674040 |
| chr10 | 120578403 | 120578453 |
| chr1 | 29219124 | 29219174 |
| chr1 | 29352010 | 29352060 |
| chr10 | 6308540 | 6308590 |
| chr1 | 30505961 | 30506011 |
| chr1 | 30646775 | 30646825 |
| chr1 | 30805080 | 30805130 |
| chr1 | 31486513 | 31486563 |
| chr1 | 31485821 | 31485871 |
| chr1 | 31977074 | 31977124 |
| chr1 | 32373377 | 32373427 |
| chr1 | 32421253 | 32421303 |
| chr1 | 32488685 | 32488735 |
| chr1 | 32889254 | 32889304 |
| chr10 | 120908809 | 120908859 |
| chr1 | 34580911 | 34580961 |
| chr1 | 35144722 | 35144772 |
| chr1 | 35190033 | 35190083 |
| chr1 | 35664897 | 35664947 |
| chr1 | 36134155 | 36134205 |
| chr1 | 36407907 | 36407957 |
| chr1 | 36622726 | 36622776 |
| chr1 | 36631178 | 36631228 |
| chr1 | 36805534 | 36805584 |
| chr1 | 36805267 | 36805317 |
| chr10 | 121195738 | 121195788 |
| chr10 | 121231194 | 121231244 |
| chr1 | 38666223 | 38666273 |
| chr1 | 38805488 | 38805538 |
| chr1 | 38838860 | 38838910 |
| chr1 | 38893291 | 38893341 |
| chr1 | 39483892 | 39483942 |
| chr1 | 39488592 | 39488642 |
| chr1 | 39592607 | 39592657 |
| chr1 | 39594422 | 39594472 |
| chr1 | 40029438 | 40029488 |
| chr1 | 40154072 | 40154122 |
| chr1 | 40216538 | 40216588 |
| chr10 | 121567388 | 121567438 |
| chr1 | 41149874 | 41149924 |
| chr1 | 42075344 | 42075394 |
| chr1 | 42224902 | 42224952 |
| chr1 | 42474148 | 42474198 |
| chr1 | 42499910 | 42499960 |
| chr1 | 43350326 | 43350376 |
| chr1 | 43527179 | 43527229 |
| chr1 | 43598309 | 43598359 |
| chr1 | 43958837 | 43958887 |
| chr1 | 45069732 | 45069782 |
| chr1 | 45091098 | 45091148 |
| chr10 | 122634166 | 122634216 |
| chr1 | 45637040 | 45637090 |
| chr1 | 45909504 | 45909554 |
| chr1 | 47497168 | 47497218 |
| chr1 | 47645938 | 47645988 |
| chr1 | 48281423 | 48281473 |
| chr1 | 49242874 | 49242924 |
| chr1 | 49446852 | 49446902 |
| chr1 | 49623259 | 49623309 |
| chr1 | 49757364 | 49757414 |
| chr1 | 49890714 | 49890764 |
| chr1 | 50233894 | 50233944 |
| chr1 | 50755344 | 50755394 |
| chr1 | 50827856 | 50827906 |
| chr1 | 50867854 | 50867904 |
| chr1 | 51735677 | 51735727 |
| chr1 | 51990760 | 51990810 |
| chr1 | 51992708 | 51992758 |
| chr1 | 51994663 | 51994713 |
| chr1 | 52280202 | 52280252 |
| chr1 | 52502080 | 52502130 |
| chr10 | 123151398 | 123151448 |
| chr1 | 53009349 | 53009399 |
| chr1 | 53727746 | 53727796 |
| chr1 | 53941130 | 53941180 |
| chr1 | 53990880 | 53990930 |
| chr1 | 54267898 | 54267948 |
| chr1 | 54294861 | 54294911 |
| chr1 | 54539328 | 54539378 |
| chr1 | 56885089 | 56885139 |
| chr1 | 57043366 | 57043416 |
| chr1 | 57141076 | 57141126 |
| chr1 | 57405900 | 57405950 |
| chr1 | 57438378 | 57438428 |
| chr1 | 57666817 | 57666867 |
| chr1 | 57892214 | 57892264 |
| chr1 | 57892144 | 57892194 |
| chr1 | 58990315 | 58990365 |
| chr1 | 61307224 | 61307274 |
| chr1 | 61327631 | 61327681 |
| chr1 | 62087267 | 62087317 |
| chr1 | 62253220 | 62253270 |
| chr1 | 62372854 | 62372904 |
| chr1 | 62916460 | 62916510 |
| chr1 | 63073556 | 63073606 |
| chr1 | 64425593 | 64425643 |
| chr1 | 64729881 | 64729931 |
| chr1 | 64812484 | 64812534 |
| chr1 | 64928256 | 64928306 |
| chr1 | 64973506 | 64973556 |
| chr1 | 66045822 | 66045872 |
| chr1 | 66660569 | 66660619 |
| chr1 | 68646466 | 68646516 |
| chr1 | 68654547 | 68654597 |
| chr1 | 68654802 | 68654852 |
| chr1 | 68728063 | 68728113 |
| chr1 | 69011140 | 69011190 |
| chr1 | 70092293 | 70092343 |
| chr1 | 70092762 | 70092812 |
| chr1 | 70359641 | 70359691 |
| chr1 | 71962579 | 71962629 |
| chr1 | 72392831 | 72392881 |
| chr1 | 74785970 | 74786020 |
| chr1 | 74845705 | 74845755 |
| chr1 | 75315196 | 75315246 |
| chr1 | 76562069 | 76562119 |
| chr1 | 79029411 | 79029461 |
| chr1 | 79356605 | 79356655 |
| chr1 | 79669665 | 79669715 |
| chr1 | 79938727 | 79938777 |
| chr1 | 79938965 | 79939015 |
| chr1 | 79939292 | 79939342 |
| chr1 | 79939829 | 79939879 |
| chr1 | 79940070 | 79940120 |
| chr1 | 80616912 | 80616962 |
| chr1 | 80631292 | 80631342 |
| chr1 | 82052362 | 82052412 |
| chr1 | 82441539 | 82441589 |
| chr1 | 82706259 | 82706309 |
| chr1 | 82896747 | 82896797 |
| chr1 | 83094883 | 83094933 |
| chr10 | 125009785 | 125009835 |
| chr1 | 84424662 | 84424712 |
| chr1 | 84425000 | 84425050 |
| chr1 | 84425437 | 84425487 |
| chr1 | 84425812 | 84425862 |
| chr1 | 84523374 | 84523424 |
| chr1 | 84523108 | 84523158 |
| chr1 | 85280755 | 85280805 |
| chr1 | 85702192 | 85702242 |
| chr1 | 86741831 | 86741881 |
| chr1 | 87300282 | 87300332 |
| chr1 | 87369188 | 87369238 |
| chr1 | 87653982 | 87654032 |
| chr1 | 87979260 | 87979310 |
| chr1 | 88529129 | 88529179 |
| chr1 | 88570535 | 88570585 |
| chr1 | 90559775 | 90559825 |
| chr1 | 90735117 | 90735167 |
| chr10 | 6732685 | 6732735 |
| chr1 | 91027734 | 91027784 |
| chr1 | 91068049 | 91068099 |
| chr1 | 91068263 | 91068313 |
| chr1 | 91068479 | 91068529 |
| chr1 | 91068828 | 91068878 |
| chr1 | 91524572 | 91524622 |
| chr1 | 92051058 | 92051108 |
| chr1 | 92100398 | 92100448 |
| chr1 | 93434106 | 93434156 |
| chr1 | 94104696 | 94104746 |
| chr1 | 94104452 | 94104502 |
| chr1 | 94531472 | 94531522 |
| chr1 | 94531669 | 94531719 |
| chr1 | 94531332 | 94531382 |
| chr1 | 94615214 | 94615264 |
| chr1 | 95190210 | 95190260 |
| chr1 | 95191644 | 95191694 |
| chr1 | 96184470 | 96184520 |
| chr1 | 98585743 | 98585793 |
| chr1 | 98588125 | 98588175 |
| chr1 | 98870944 | 98870994 |
| chr10 | 6800392 | 6800442 |
| chr1 | 99741243 | 99741293 |
| chr1 | 99741527 | 99741577 |
| chr1 | 99741612 | 99741662 |
| chr10 | 6825607 | 6825657 |
| chr1 | 100673040 | 100673090 |
| chr10 | 6825186 | 6825236 |
| chr1 | 102558816 | 102558866 |
| chr1 | 103056319 | 103056369 |
| chr10 | 125859633 | 125859683 |
| chr1 | 103974751 | 103974801 |
| chr1 | 104698243 | 104698293 |
| chr1 | 105493440 | 105493490 |
| chr1 | 107467789 | 107467839 |
| chr10 | 125933926 | 125933976 |
| chr1 | 108558282 | 108558332 |
| chr1 | 108845441 | 108845491 |
| chr1 | 109279029 | 109279079 |
| chr1 | 111132469 | 111132519 |
| chr1 | 111132109 | 111132159 |
| chr1 | 112613929 | 112613979 |
| chr1 | 112631106 | 112631156 |
| chr1 | 113907964 | 113908014 |
| chr1 | 114308052 | 114308102 |
| chr1 | 114358835 | 114358885 |
| chr10 | 7038825 | 7038875 |
| chr1 | 117265237 | 117265287 |
| chr1 | 117597744 | 117597794 |
| chr1 | 117710217 | 117710267 |
| chr1 | 117710017 | 117710067 |
| chr1 | 118231833 | 118231883 |
| chr1 | 118919477 | 118919527 |
| chr1 | 119973468 | 119973518 |
| chr1 | 140800540 | 140800590 |
| chr1 | 140876147 | 140876197 |
| chr10 | 126393843 | 126393893 |
| chr10 | 126393974 | 126394024 |
| chr1 | 141717898 | 141717948 |
| chr1 | 143232851 | 143232901 |
| chr10 | 126437559 | 126437609 |
| chr1 | 145434524 | 145434574 |
| chr1 | 145504231 | 145504281 |
| chr1 | 146023401 | 146023451 |
| chr1 | 146301886 | 146301936 |
| chr1 | 146555801 | 146555851 |
| chr1 | 146586234 | 146586284 |
| chr1 | 146729526 | 146729576 |
| chr1 | 147355856 | 147355906 |
| chr1 | 147428603 | 147428653 |
| chr1 | 148290982 | 148291032 |
| chr10 | 126888891 | 126888941 |
| chr10 | 126888707 | 126888757 |
| chr1 | 148687564 | 148687614 |
| chr1 | 148691338 | 148691388 |
| chr1 | 149110062 | 149110112 |
| chr1 | 149160920 | 149160970 |
| chr10 | 127045012 | 127045062 |
| chr1 | 150060566 | 150060616 |
| chr1 | 150109199 | 150109249 |
| chr10 | 127124121 | 127124171 |
| chr1 | 151491150 | 151491200 |
| chr1 | 151699928 | 151699978 |
| chr1 | 152351055 | 152351105 |
| chr10 | 7225709 | 7225759 |
| chr1 | 153539296 | 153539346 |
| chr1 | 153598098 | 153598148 |
| chr1 | 154011756 | 154011806 |
| chr1 | 154757518 | 154757568 |
| chr1 | 155390721 | 155390771 |
| chr1 | 155390950 | 155391000 |
| chr1 | 155391288 | 155391338 |
| chr1 | 155391377 | 155391427 |
| chr10 | 128141758 | 128141808 |
| chr1 | 156951066 | 156951116 |
| chr1 | 158619615 | 158619665 |
| chr1 | 158619006 | 158619056 |
| chr1 | 160164380 | 160164430 |
| chr1 | 162047623 | 162047673 |
| chr1 | 162973836 | 162973886 |
| chr1 | 164040876 | 164040926 |
| chr1 | 164685897 | 164685947 |
| chr1 | 166833898 | 166833948 |
| chr1 | 168094323 | 168094373 |
| chr1 | 168093758 | 168093808 |
| chr1 | 168817220 | 168817270 |
| chr1 | 168895638 | 168895688 |
| chr1 | 169956782 | 169956832 |
| chr1 | 170471915 | 170471965 |
| chr10 | 128755205 | 128755255 |
| chr1 | 171299696 | 171299746 |
| chr10 | 128829814 | 128829864 |
| chr10 | 128829745 | 128829795 |
| chr1 | 173017001 | 173017051 |
| chr1 | 174633772 | 174633822 |
| chr1 | 175962612 | 175962662 |
| chr1 | 176277764 | 176277814 |
| chr1 | 176594331 | 176594381 |
| chr10 | 129223436 | 129223486 |
| chr1 | 178387501 | 178387551 |
| chr1 | 179824967 | 179825017 |
| chr1 | 182915854 | 182915904 |
| chr1 | 183645133 | 183645183 |
| chr1 | 183644929 | 183644979 |
| chr1 | 183918698 | 183918748 |
| chr1 | 184282640 | 184282690 |
| chr1 | 184756872 | 184756922 |
| chr1 | 185208980 | 185209030 |
| chr1 | 185214345 | 185214395 |
| chr1 | 185518050 | 185518100 |
| chr1 | 185533348 | 185533398 |
| chr1 | 185549613 | 185549663 |
| chr1 | 185950123 | 185950173 |
| chr1 | 186015757 | 186015807 |
| chr1 | 186655685 | 186655735 |
| chr1 | 186732464 | 186732514 |
| chr1 | 187126691 | 187126741 |
| chr1 | 188566672 | 188566722 |
| chr10 | 129677949 | 129677999 |
| chr1 | 189818778 | 189818828 |
| chr1 | 190095057 | 190095107 |
| chr1 | 190665709 | 190665759 |
| chr1 | 192085481 | 192085531 |
| chr1 | 192262010 | 192262060 |
| chr1 | 192561402 | 192561452 |
| chr10 | 129785596 | 129785646 |
| chr1 | 195349638 | 195349688 |
| chr1 | 196235232 | 196235282 |
| chr1 | 196924509 | 196924559 |
| chr1 | 197153462 | 197153512 |
| chr1 | 197212030 | 197212080 |
| chr1 | 198193523 | 198193573 |
| chr1 | 198839665 | 198839715 |
| chr1 | 199327261 | 199327311 |
| chr1 | 200592354 | 200592404 |
| chr1 | 200758353 | 200758403 |
| chr1 | 200863454 | 200863504 |
| chr1 | 200934889 | 200934939 |
| chr1 | 201643013 | 201643063 |
| chr1 | 204232978 | 204233028 |
| chr1 | 204680138 | 204680188 |
| chr1 | 204680639 | 204680689 |
| chr1 | 204681551 | 204681601 |
| chr1 | 206330564 | 206330614 |
| chr1 | 206700603 | 206700653 |
| chr1 | 207461963 | 207462013 |
| chr1 | 207754800 | 207754850 |
| chr1 | 208377402 | 208377452 |
| chr10 | 130677111 | 130677161 |
| chr1 | 208587073 | 208587123 |
| chr10 | 7537343 | 7537393 |
| chr1 | 210721957 | 210722007 |
| chr1 | 212089602 | 212089652 |
| chr1 | 212251208 | 212251258 |
| chr1 | 212727499 | 212727549 |
| chr1 | 214702420 | 214702470 |
| chr1 | 215143042 | 215143092 |
| chr1 | 215312336 | 215312386 |
| chr1 | 217093389 | 217093439 |
| chr1 | 217311393 | 217311443 |
| chr1 | 218416783 | 218416833 |
| chr1 | 218448714 | 218448764 |
| chr1 | 219280599 | 219280649 |
| chr1 | 220036870 | 220036920 |
| chr1 | 220589457 | 220589507 |
| chr1 | 221698381 | 221698431 |
| chr1 | 221734090 | 221734140 |
| chr1 | 221999851 | 221999901 |
| chr1 | 222880686 | 222880736 |
| chr1 | 223249530 | 223249580 |
| chr1 | 223698589 | 223698639 |
| chr1 | 223705206 | 223705256 |
| chr1 | 223705042 | 223705092 |
| chr1 | 223862366 | 223862416 |
| chr1 | 224874141 | 224874191 |
| chr1 | 225070016 | 225070066 |
| chr1 | 225663620 | 225663670 |
| chr1 | 226112769 | 226112819 |
| chr1 | 226493432 | 226493482 |
| chr1 | 226702304 | 226702354 |
| chr1 | 226793704 | 226793754 |
| chr1 | 227269693 | 227269743 |
| chr1 | 227752558 | 227752608 |
| chr1 | 228393587 | 228393637 |
| chr1 | 228742319 | 228742369 |
| chr10 | 131335709 | 131335759 |
| chr1 | 230159655 | 230159705 |
| chr1 | 230815266 | 230815316 |
| chr1 | 232014621 | 232014671 |
| chr1 | 232321284 | 232321334 |
| chr1 | 233341706 | 233341756 |
| chr1 | 233736058 | 233736108 |
| chr1 | 233736976 | 233737026 |
| chr1 | 234259612 | 234259662 |
| chr1 | 235443317 | 235443367 |
| chr1 | 235473221 | 235473271 |
| chr10 | 131824919 | 131824969 |
| chr1 | 235600131 | 235600181 |
| chr1 | 235834536 | 235834586 |
| chr1 | 236099134 | 236099184 |
| chr1 | 237153638 | 237153688 |
| chr10 | 131932323 | 131932373 |
| chr1 | 237845055 | 237845105 |
| chr1 | 238027964 | 238028014 |
| chr10 | 132083157 | 132083207 |
| chr1 | 240132156 | 240132206 |
| chr10 | 132134256 | 132134306 |
| chr10 | 132158267 | 132158317 |
| chr1 | 240975151 | 240975201 |
| chr10 | 132210821 | 132210871 |
| chr10 | 7697695 | 7697745 |
| chr1 | 241197888 | 241197938 |
| chr10 | 132235941 | 132235991 |
| chr1 | 241460664 | 241460714 |
| chr1 | 241503780 | 241503830 |
| chr1 | 241508439 | 241508489 |
| chr1 | 241750911 | 241750961 |
| chr1 | 241803821 | 241803871 |
| chr1 | 241809257 | 241809307 |
| chr1 | 241867287 | 241867337 |
| chr1 | 242173630 | 242173680 |
| chr1 | 242244752 | 242244802 |
| chr1 | 242419138 | 242419188 |
| chr1 | 242575773 | 242575823 |
| chr10 | 132361754 | 132361804 |
| chr1 | 243455869 | 243455919 |
| chr1 | 243619604 | 243619654 |
| chr1 | 243655191 | 243655241 |
| chr10 | 132388010 | 132388060 |
| chr1 | 243793556 | 243793606 |
| chr1 | 243943615 | 243943665 |
| chr1 | 244141539 | 244141589 |
| chr1 | 244143609 | 244143659 |
| chr1 | 244255088 | 244255138 |
| chr1 | 244258672 | 244258722 |
| chr1 | 244446259 | 244446309 |
| chr1 | 244499146 | 244499196 |
| chr1 | 244886214 | 244886264 |
| chr1 | 245022028 | 245022078 |
| chr1 | 245891792 | 245891842 |
| chr1 | 245891674 | 245891724 |
| chr1 | 246005048 | 246005098 |
| chr20 | 243868 | 243918 |
| chr20 | 1089651 | 1089701 |
| chr20 | 1404474 | 1404524 |
| chr10 | 132537592 | 132537642 |
| chr20 | 1627121 | 1627171 |
| chr20 | 1626940 | 1626990 |
| chr20 | 2154318 | 2154368 |
| chr20 | 2327923 | 2327973 |
| chr20 | 2643440 | 2643490 |
| chr20 | 2651528 | 2651578 |
| chr20 | 2716019 | 2716069 |
| chr20 | 2827623 | 2827673 |
| chr20 | 2968225 | 2968275 |
| chr20 | 3074182 | 3074232 |
| chr20 | 3406172 | 3406222 |
| chr20 | 3508702 | 3508752 |
| chr20 | 3748977 | 3749027 |
| chr20 | 3999437 | 3999487 |
| chr20 | 4052768 | 4052818 |
| chr20 | 4067569 | 4067619 |
| chr20 | 4078369 | 4078419 |
| chr20 | 4080502 | 4080552 |
| chr20 | 4082818 | 4082868 |
| chr20 | 4103397 | 4103447 |
| chr20 | 4178279 | 4178329 |
| chr20 | 4741141 | 4741191 |
| chr20 | 4985317 | 4985367 |
| chr20 | 5032432 | 5032482 |
| chr20 | 5653274 | 5653324 |
| chr20 | 5667505 | 5667555 |
| chr20 | 5829188 | 5829238 |
| chr20 | 5971609 | 5971659 |
| chr20 | 6366207 | 6366257 |
| chr20 | 6833036 | 6833086 |
| chr20 | 7708228 | 7708278 |
| chr20 | 8223898 | 8223948 |
| chr20 | 8223736 | 8223786 |
| chr20 | 8874534 | 8874584 |
| chr20 | 9587813 | 9587863 |
| chr20 | 10238547 | 10238597 |
| chr20 | 10634097 | 10634147 |
| chr20 | 10783777 | 10783827 |
| chr20 | 11372729 | 11372779 |
| chr20 | 11691758 | 11691808 |
| chr20 | 11766864 | 11766914 |
| chr20 | 11841858 | 11841908 |
| chr20 | 12446009 | 12446059 |
| chr20 | 12490909 | 12490959 |
| chr20 | 13668055 | 13668105 |
| chr20 | 13816397 | 13816447 |
| chr20 | 13901648 | 13901698 |
| chr20 | 14183029 | 14183079 |
| chr20 | 14461538 | 14461588 |
| chr20 | 14524529 | 14524579 |
| chr20 | 15897119 | 15897169 |
| chr20 | 15999210 | 15999260 |
| chr20 | 17456581 | 17456631 |
| chr20 | 17789508 | 17789558 |
| chr20 | 17817112 | 17817162 |
| chr20 | 17995380 | 17995430 |
| chr20 | 18001306 | 18001356 |
| chr20 | 18068081 | 18068131 |
| chr20 | 18769665 | 18769715 |
| chr20 | 18769845 | 18769895 |
| chr20 | 18783848 | 18783898 |
| chr20 | 19195173 | 19195223 |
| chr20 | 19256626 | 19256676 |
| chr20 | 19256468 | 19256518 |
| chr20 | 19470875 | 19470925 |
| chr20 | 19470656 | 19470706 |
| chr20 | 19486777 | 19486827 |
| chr20 | 19627186 | 19627236 |
| chr20 | 19629691 | 19629741 |
| chr10 | 133279044 | 133279094 |
| chr20 | 20678000 | 20678050 |
| chr20 | 21047657 | 21047707 |
| chr20 | 21420514 | 21420564 |
| chr20 | 21705167 | 21705217 |
| chr20 | 21704982 | 21705032 |
| chr20 | 21900122 | 21900172 |
| chr20 | 22127357 | 22127407 |
| chr20 | 22574365 | 22574415 |
| chr20 | 22660621 | 22660671 |
| chr20 | 22660355 | 22660405 |
| chr20 | 22977155 | 22977205 |
| chr20 | 22982692 | 22982742 |
| chr20 | 23062995 | 23063045 |
| chr20 | 23422199 | 23422249 |
| chr20 | 23466696 | 23466746 |
| chr20 | 24554172 | 24554222 |
| chr10 | 133428372 | 133428422 |
| chr20 | 30867132 | 30867182 |
| chr20 | 30986959 | 30987009 |
| chr10 | 133486292 | 133486342 |
| chr20 | 32564266 | 32564316 |
| chr20 | 32800985 | 32801035 |
| chr20 | 33159143 | 33159193 |
| chr20 | 33538013 | 33538063 |
| chr20 | 33643656 | 33643706 |
| chr10 | 133559452 | 133559502 |
| chr20 | 34412449 | 34412499 |
| chr20 | 34483400 | 34483450 |
| chr20 | 35723882 | 35723932 |
| chr20 | 35725173 | 35725223 |
| chr20 | 35737842 | 35737892 |
| chr20 | 35953103 | 35953153 |
| chr10 | 133682067 | 133682117 |
| chr20 | 36103118 | 36103168 |
| chr20 | 36112264 | 36112314 |
| chr20 | 36256919 | 36256969 |
| chr20 | 36386381 | 36386431 |
| chr20 | 36391236 | 36391286 |
| chr20 | 36885971 | 36886021 |
| chr20 | 36924329 | 36924379 |
| chr20 | 36997131 | 36997181 |
| chr20 | 37358484 | 37358534 |
| chr20 | 37694101 | 37694151 |
| chr20 | 37694043 | 37694093 |
| chr20 | 37969871 | 37969921 |
| chr20 | 38390453 | 38390503 |
| chr20 | 38742153 | 38742203 |
| chr20 | 38819610 | 38819660 |
| chr20 | 38860263 | 38860313 |
| chr20 | 39722730 | 39722780 |
| chr20 | 40269085 | 40269135 |
| chr20 | 40268994 | 40269044 |
| chr20 | 40945275 | 40945325 |
| chr20 | 41029425 | 41029475 |
| chr20 | 41089142 | 41089192 |
| chr20 | 41330602 | 41330652 |
| chr20 | 41754886 | 41754936 |
| chr20 | 42000466 | 42000516 |
| chr20 | 42385536 | 42385586 |
| chr20 | 42532152 | 42532202 |
| chr20 | 42565910 | 42565960 |
| chr20 | 42653536 | 42653586 |
| chr20 | 43004187 | 43004237 |
| chr20 | 43224124 | 43224174 |
| chr20 | 43411404 | 43411454 |
| chr20 | 43429975 | 43430025 |
| chr20 | 43794463 | 43794513 |
| chr20 | 43880192 | 43880242 |
| chr20 | 44598992 | 44599042 |
| chr20 | 44808270 | 44808320 |
| chr20 | 45312362 | 45312412 |
| chr20 | 45312251 | 45312301 |
| chr20 | 45423244 | 45423294 |
| chr10 | 133920653 | 133920703 |
| chr20 | 45783368 | 45783418 |
| chr20 | 46162025 | 46162075 |
| chr20 | 46388279 | 46388329 |
| chr20 | 46388212 | 46388262 |
| chr20 | 46726168 | 46726218 |
| chr20 | 46773608 | 46773658 |
| chr20 | 47156454 | 47156504 |
| chr20 | 47165361 | 47165411 |
| chr20 | 47182101 | 47182151 |
| chr20 | 47194089 | 47194139 |
| chr20 | 47209101 | 47209151 |
| chr20 | 47212140 | 47212190 |
| chr20 | 47211823 | 47211873 |
| chr20 | 47265273 | 47265323 |
| chr20 | 47435851 | 47435901 |
| chr20 | 47509103 | 47509153 |
| chr20 | 47508917 | 47508967 |
| chr20 | 47511095 | 47511145 |
| chr20 | 47657627 | 47657677 |
| chr20 | 47670203 | 47670253 |
| chr20 | 47745450 | 47745500 |
| chr20 | 47772098 | 47772148 |
| chr20 | 47771951 | 47772001 |
| chr20 | 47785934 | 47785984 |
| chr20 | 47787367 | 47787417 |
| chr20 | 47818208 | 47818258 |
| chr20 | 47842721 | 47842771 |
| chr20 | 47854413 | 47854463 |
| chr20 | 48755094 | 48755144 |
| chr20 | 49474599 | 49474649 |
| chr20 | 49588600 | 49588650 |
| chr20 | 50250050 | 50250100 |
| chr20 | 50629744 | 50629794 |
| chr20 | 52715275 | 52715325 |
| chr20 | 53027156 | 53027206 |
| chr20 | 53137589 | 53137639 |
| chr20 | 53138979 | 53139029 |
| chr20 | 53285572 | 53285622 |
| chr20 | 53285367 | 53285417 |
| chr20 | 53731558 | 53731608 |
| chr20 | 54833663 | 54833713 |
| chr20 | 55005483 | 55005533 |
| chr10 | 134251419 | 134251469 |
| chr20 | 55773201 | 55773251 |
| chr20 | 55952755 | 55952805 |
| chr20 | 55956411 | 55956461 |
| chr20 | 56355733 | 56355783 |
| chr20 | 57055484 | 57055534 |
| chr20 | 57071310 | 57071360 |
| chr20 | 57128629 | 57128679 |
| chr20 | 58055850 | 58055900 |
| chr20 | 58205698 | 58205748 |
| chr20 | 58396502 | 58396552 |
| chr20 | 58699647 | 58699697 |
| chr20 | 58699522 | 58699572 |
| chr20 | 58701067 | 58701117 |
| chr20 | 59741396 | 59741446 |
| chr20 | 59741305 | 59741355 |
| chr20 | 59900274 | 59900324 |
| chr20 | 59976389 | 59976439 |
| chr20 | 60658952 | 60659002 |
| chr20 | 61165970 | 61166020 |
| chr20 | 61485106 | 61485156 |
| chr20 | 62055084 | 62055134 |
| chr20 | 62129866 | 62129916 |
| chr20 | 62214453 | 62214503 |
| chr20 | 62233155 | 62233205 |
| chr20 | 62405738 | 62405788 |
| chr20 | 62472267 | 62472317 |
| chr10 | 134663497 | 134663547 |
| chr20 | 63116141 | 63116191 |
| chr21 | 10198668 | 10198718 |
| chr21 | 16496384 | 16496434 |
| chr21 | 17621576 | 17621626 |
| chr21 | 17698372 | 17698422 |
| chr21 | 17922576 | 17922626 |
| chr21 | 18211675 | 18211725 |
| chr21 | 18211411 | 18211461 |
| chr21 | 18418060 | 18418110 |
| chr21 | 19114566 | 19114616 |
| chr21 | 19331919 | 19331969 |
| chr21 | 19441032 | 19441082 |
| chr21 | 19579152 | 19579202 |
| chr21 | 19647377 | 19647427 |
| chr21 | 20090088 | 20090138 |
| chr21 | 20118061 | 20118111 |
| chr21 | 20417987 | 20418037 |
| chr21 | 20736119 | 20736169 |
| chr21 | 20737501 | 20737551 |
| chr11 | 249897 | 249947 |
| chr21 | 21026735 | 21026785 |
| chr21 | 21028477 | 21028527 |
| chr21 | 22016893 | 22016943 |
| chr21 | 22190325 | 22190375 |
| chr21 | 22392827 | 22392877 |
| chr21 | 22392663 | 22392713 |
| chr21 | 22587274 | 22587324 |
| chr21 | 22863933 | 22863983 |
| chr21 | 23364093 | 23364143 |
| chr21 | 23914519 | 23914569 |
| chr21 | 24198849 | 24198899 |
| chr21 | 24859870 | 24859920 |
| chr21 | 25200111 | 25200161 |
| chr21 | 25320550 | 25320600 |
| chr21 | 25959866 | 25959916 |
| chr21 | 26101449 | 26101499 |
| chr11 | 352527 | 352577 |
| chr21 | 26125060 | 26125110 |
| chr11 | 352986 | 353036 |
| chr11 | 353037 | 353087 |
| chr11 | 354746 | 354796 |
| chr11 | 356079 | 356129 |
| chr21 | 26577982 | 26578032 |
| chr21 | 26700200 | 26700250 |
| chr21 | 26700024 | 26700074 |
| chr21 | 27035141 | 27035191 |
| chr21 | 27291750 | 27291800 |
| chr21 | 28175754 | 28175804 |
| chr21 | 28595088 | 28595138 |
| chr21 | 28726488 | 28726538 |
| chr21 | 29145010 | 29145060 |
| chr21 | 29156309 | 29156359 |
| chr21 | 29169285 | 29169335 |
| chr21 | 29169145 | 29169195 |
| chr21 | 30735351 | 30735401 |
| chr21 | 30734831 | 30734881 |
| chr21 | 31078199 | 31078249 |
| chr21 | 31126921 | 31126971 |
| chr21 | 31486787 | 31486837 |
| chr21 | 31741048 | 31741098 |
| chr21 | 31906302 | 31906352 |
| chr21 | 32173891 | 32173941 |
| chr21 | 32498097 | 32498147 |
| chr21 | 32576833 | 32576883 |
| chr21 | 32576749 | 32576799 |
| chr21 | 33118597 | 33118647 |
| chr21 | 33583440 | 33583490 |
| chr21 | 33678889 | 33678939 |
| chr21 | 36846468 | 36846518 |
| chr21 | 37584702 | 37584752 |
| chr21 | 37597268 | 37597318 |
| chr21 | 37877625 | 37877675 |
| chr21 | 38413841 | 38413891 |
| chr21 | 38676102 | 38676152 |
| chr21 | 39010314 | 39010364 |
| chr21 | 39015271 | 39015321 |
| chr21 | 40004302 | 40004352 |
| chr21 | 40723245 | 40723295 |
| chr21 | 40782219 | 40782269 |
| chr21 | 40909024 | 40909074 |
| chr21 | 41064962 | 41065012 |
| chr21 | 41170710 | 41170760 |
| chr21 | 41253213 | 41253263 |
| chr21 | 41296555 | 41296605 |
| chr21 | 41414310 | 41414360 |
| chr21 | 41749015 | 41749065 |
| chr21 | 42853977 | 42854027 |
| chr21 | 43081455 | 43081505 |
| chr21 | 43158857 | 43158907 |
| chr21 | 43369370 | 43369420 |
| chr21 | 43627781 | 43627831 |
| chr21 | 43664260 | 43664310 |
| chr21 | 43747724 | 43747774 |
| chr21 | 43747504 | 43747554 |
| chr21 | 44755927 | 44755977 |
| chr21 | 44878855 | 44878905 |
| chr21 | 44928959 | 44929009 |
| chr21 | 45567229 | 45567279 |
| chr11 | 1056228 | 1056278 |
| chr21 | 45665250 | 45665300 |
| chr21 | 45907092 | 45907142 |
| chr21 | 46343979 | 46344029 |
| chr21 | 46371609 | 46371659 |
| chr21 | 46435966 | 46436016 |
| chr21 | 46615321 | 46615371 |
| chr21 | 46727637 | 46727687 |
| chr21 | 46887091 | 46887141 |
| chr22 | 15312379 | 15312429 |
| chr22 | 16142004 | 16142054 |
| chr22 | 16158992 | 16159042 |
| chr22 | 16193335 | 16193385 |
| chr22 | 17463493 | 17463543 |
| chr22 | 17517375 | 17517425 |
| chr11 | 1351881 | 1351931 |
| chr22 | 18002057 | 18002107 |
| chr22 | 20676585 | 20676635 |
| chr22 | 20903477 | 20903527 |
| chr22 | 20903565 | 20903615 |
| chr22 | 21064093 | 21064143 |
| chr22 | 21067166 | 21067216 |
| chr22 | 21274136 | 21274186 |
| chr22 | 21432174 | 21432224 |
| chr22 | 21621677 | 21621727 |
| chr11 | 1527899 | 1527949 |
| chr22 | 22386221 | 22386271 |
| chr22 | 22422665 | 22422715 |
| chr22 | 22424320 | 22424370 |
| chr22 | 22538694 | 22538744 |
| chr22 | 22540063 | 22540113 |
| chr22 | 22601302 | 22601352 |
| chr22 | 22623362 | 22623412 |
| chr11 | 1601856 | 1601906 |
| chr22 | 23523570 | 23523620 |
| chr22 | 23763329 | 23763379 |
| chr22 | 23908895 | 23908945 |
| chr22 | 23910026 | 23910076 |
| chr10 | 9222888 | 9222938 |
| chr11 | 1646489 | 1646539 |
| chr11 | 1646723 | 1646773 |
| chr22 | 24456416 | 24456466 |
| chr22 | 24459304 | 24459354 |
| chr22 | 25128439 | 25128489 |
| chr22 | 25370486 | 25370536 |
| chr22 | 25410597 | 25410647 |
| chr22 | 25441514 | 25441564 |
| chr10 | 9262698 | 9262748 |
| chr22 | 25915919 | 25915969 |
| chr22 | 26159202 | 26159252 |
| chr22 | 26820350 | 26820400 |
| chr22 | 27460314 | 27460364 |
| chr22 | 27466979 | 27467029 |
| chr22 | 27467628 | 27467678 |
| chr22 | 27485279 | 27485329 |
| chr11 | 1793868 | 1793918 |
| chr22 | 28117736 | 28117786 |
| chr22 | 29452407 | 29452457 |
| chr22 | 30329895 | 30329945 |
| chr22 | 30576231 | 30576281 |
| chr22 | 31055246 | 31055296 |
| chr22 | 31055099 | 31055149 |
| chr22 | 31477700 | 31477750 |
| chr22 | 32080385 | 32080435 |
| chr22 | 32081435 | 32081485 |
| chr22 | 32255216 | 32255266 |
| chr11 | 1993550 | 1993600 |
| chr22 | 32646888 | 32646938 |
| chr22 | 33386520 | 33386570 |
| chr22 | 33677036 | 33677086 |
| chr22 | 34302719 | 34302769 |
| chr22 | 34619426 | 34619476 |
| chr22 | 34772460 | 34772510 |
| chr22 | 34803361 | 34803411 |
| chr22 | 34848163 | 34848213 |
| chr22 | 34854290 | 34854340 |
| chr22 | 34860347 | 34860397 |
| chr22 | 34934904 | 34934954 |
| chr22 | 35432316 | 35432366 |
| chr22 | 35621206 | 35621256 |
| chr22 | 35736818 | 35736868 |
| chr22 | 35872781 | 35872831 |
| chr11 | 2223264 | 2223314 |
| chr11 | 2280080 | 2280130 |
| chr22 | 37245908 | 37245958 |
| chr11 | 2340261 | 2340311 |
| chr22 | 37402223 | 37402273 |
| chr11 | 2343382 | 2343432 |
| chr11 | 2346107 | 2346157 |
| chr22 | 37486558 | 37486608 |
| chr22 | 37642942 | 37642992 |
| chr22 | 37765871 | 37765921 |
| chr22 | 37853551 | 37853601 |
| chr22 | 37945312 | 37945362 |
| chr22 | 38338119 | 38338169 |
| chr22 | 39298183 | 39298233 |
| chr22 | 39303942 | 39303992 |
| chr22 | 39311052 | 39311102 |
| chr22 | 39381226 | 39381276 |
| chr22 | 39559437 | 39559487 |
| chr22 | 39667088 | 39667138 |
| chr22 | 39670712 | 39670762 |
| chr22 | 39935942 | 39935992 |
| chr22 | 40308774 | 40308824 |
| chr22 | 40348524 | 40348574 |
| chr22 | 40350458 | 40350508 |
| chr22 | 40454531 | 40454581 |
| chr10 | 10032530 | 10032580 |
| chr22 | 41077304 | 41077354 |
| chr22 | 41147587 | 41147637 |
| chr22 | 41148379 | 41148429 |
| chr22 | 41153900 | 41153950 |
| chr22 | 41155029 | 41155079 |
| chr22 | 41179450 | 41179500 |
| chr22 | 41298475 | 41298525 |
| chr22 | 41326511 | 41326561 |
| chr22 | 41371678 | 41371728 |
| chr22 | 42165795 | 42165845 |
| chr22 | 42169471 | 42169521 |
| chr22 | 42198986 | 42199036 |
| chr22 | 42291160 | 42291210 |
| chr22 | 42411778 | 42411828 |
| chr22 | 42710174 | 42710224 |
| chr22 | 42924448 | 42924498 |
| chr22 | 43223237 | 43223287 |
| chr22 | 43338093 | 43338143 |
| chr22 | 43633345 | 43633395 |
| chr22 | 44201421 | 44201471 |
| chr22 | 44227538 | 44227588 |
| chr22 | 44231708 | 44231758 |
| chr22 | 44256527 | 44256577 |
| chr22 | 44286645 | 44286695 |
| chr22 | 44323194 | 44323244 |
| chr22 | 44532882 | 44532932 |
| chr22 | 45037605 | 45037655 |
| chr22 | 45121278 | 45121328 |
| chr22 | 45365817 | 45365867 |
| chr22 | 45600005 | 45600055 |
| chr22 | 45600670 | 45600720 |
| chr22 | 45935208 | 45935258 |
| chr22 | 45965648 | 45965698 |
| chr22 | 46038857 | 46038907 |
| chr22 | 46239906 | 46239956 |
| chr22 | 46239663 | 46239713 |
| chr22 | 46242729 | 46242779 |
| chr22 | 46556098 | 46556148 |
| chr22 | 47172109 | 47172159 |
| chr22 | 47417932 | 47417982 |
| chr22 | 47519185 | 47519235 |
| chr22 | 47538539 | 47538589 |
| chr22 | 47693651 | 47693701 |
| chr22 | 47712658 | 47712708 |
| chr22 | 47714328 | 47714378 |
| chr22 | 47890930 | 47890980 |
| chr22 | 47913364 | 47913414 |
| chr11 | 3403708 | 3403758 |
| chr11 | 3403613 | 3403663 |
| chr22 | 48190217 | 48190267 |
| chr22 | 48338196 | 48338246 |
| chr22 | 48699426 | 48699476 |
| chr22 | 49154381 | 49154431 |
| chr22 | 49155092 | 49155142 |
| chr22 | 49227911 | 49227961 |
| chrX | 2435691 | 2435741 |
| chrX | 2468586 | 2468636 |
| chrX | 2878703 | 2878753 |
| chrX | 3066239 | 3066289 |
| chrX | 3166567 | 3166617 |
| chrX | 4151370 | 4151420 |
| chrX | 4153605 | 4153655 |
| chrX | 4764063 | 4764113 |
| chr10 | 10581271 | 10581321 |
| chrX | 6372101 | 6372151 |
| chrX | 7545724 | 7545774 |
| chrX | 7550883 | 7550933 |
| chrX | 7744765 | 7744815 |
| chrX | 8224838 | 8224888 |
| chrX | 8471941 | 8471991 |
| chrX | 9094570 | 9094620 |
| chrX | 9188582 | 9188632 |
| chrX | 9391732 | 9391782 |
| chrX | 10458288 | 10458338 |
| chrX | 12178927 | 12178977 |
| chrX | 12178763 | 12178813 |
| chrX | 12525947 | 12525997 |
| chrX | 12892564 | 12892614 |
| chrX | 13416987 | 13417037 |
| chrX | 14730106 | 14730156 |
| chrX | 15074201 | 15074251 |
| chrX | 15167186 | 15167236 |
| chrX | 15802783 | 15802833 |
| chrX | 16506687 | 16506737 |
| chrX | 16647719 | 16647769 |
| chr11 | 4750550 | 4750600 |
| chrX | 17259909 | 17259959 |
| chrX | 17773873 | 17773923 |
| chrX | 17775455 | 17775505 |
| chrX | 17784709 | 17784759 |
| chrX | 17859254 | 17859304 |
| chrX | 18434567 | 18434617 |
| chrX | 18443143 | 18443193 |
| chrX | 18534342 | 18534392 |
| chrX | 18760535 | 18760585 |
| chrX | 19348344 | 19348394 |
| chrX | 19416489 | 19416539 |
| chrX | 20271515 | 20271565 |
| chrX | 20386109 | 20386159 |
| chrX | 20510452 | 20510502 |
| chrX | 20734474 | 20734524 |
| chrX | 20853574 | 20853624 |
| chrX | 21118380 | 21118430 |
| chrX | 21552576 | 21552626 |
| chrX | 21552335 | 21552385 |
| chrX | 21800896 | 21800946 |
| chrX | 22501900 | 22501950 |
| chrX | 22687586 | 22687636 |
| chrX | 22934574 | 22934624 |
| chrX | 23199210 | 23199260 |
| chrX | 23495332 | 23495382 |
| chrX | 23771859 | 23771909 |
| chrX | 23786710 | 23786760 |
| chrX | 24003817 | 24003867 |
| chrX | 24638372 | 24638422 |
| chrX | 24901828 | 24901878 |
| chrX | 25598216 | 25598266 |
| chrX | 26939418 | 26939468 |
| chrX | 27048181 | 27048231 |
| chrX | 27361129 | 27361179 |
| chrX | 27929706 | 27929756 |
| chr11 | 5959931 | 5959981 |
| chr11 | 6108497 | 6108547 |
| chrX | 29879581 | 29879631 |
| chrX | 29905010 | 29905060 |
| chrX | 30001173 | 30001223 |
| chrX | 30121998 | 30122048 |
| chrX | 30392364 | 30392414 |
| chrX | 30717627 | 30717677 |
| chrX | 30730952 | 30731002 |
| chrX | 30743519 | 30743569 |
| chrX | 31059247 | 31059297 |
| chrX | 32257601 | 32257651 |
| chrX | 32273872 | 32273922 |
| chrX | 32410952 | 32411002 |
| chrX | 32556799 | 32556849 |
| chr11 | 6351533 | 6351583 |
| chrX | 33526748 | 33526798 |
| chrX | 33577955 | 33578005 |
| chrX | 33761152 | 33761202 |
| chr11 | 6369975 | 6370025 |
| chrX | 34260772 | 34260822 |
| chrX | 34401138 | 34401188 |
| chrX | 34482804 | 34482854 |
| chrX | 34524628 | 34524678 |
| chrX | 34773265 | 34773315 |
| chrX | 35258315 | 35258365 |
| chrX | 35271076 | 35271126 |
| chrX | 35862868 | 35862918 |
| chrX | 36198961 | 36199011 |
| chrX | 37120552 | 37120602 |
| chrX | 37146302 | 37146352 |
| chrX | 37636313 | 37636363 |
| chrX | 37680962 | 37681012 |
| chrX | 38605849 | 38605899 |
| chrX | 38745528 | 38745578 |
| chrX | 38786621 | 38786671 |
| chrX | 38799196 | 38799246 |
| chrX | 39219479 | 39219529 |
| chrX | 39357529 | 39357579 |
| chrX | 39363545 | 39363595 |
| chrX | 39389929 | 39389979 |
| chrX | 39406114 | 39406164 |
| chrX | 39429787 | 39429837 |
| chrX | 39429526 | 39429576 |
| chrX | 39716439 | 39716489 |
| chrX | 39720531 | 39720581 |
| chrX | 39723070 | 39723120 |
| chrX | 39724636 | 39724686 |
| chrX | 39734057 | 39734107 |
| chrX | 39734576 | 39734626 |
| chrX | 39821402 | 39821452 |
| chrX | 39877636 | 39877686 |
| chrX | 40185787 | 40185837 |
| chrX | 40216681 | 40216731 |
| chrX | 40346512 | 40346562 |
| chrX | 40831497 | 40831547 |
| chrX | 41100851 | 41100901 |
| chrX | 41803004 | 41803054 |
| chrX | 42133829 | 42133879 |
| chrX | 42137328 | 42137378 |
| chrX | 42804802 | 42804852 |
| chrX | 42804666 | 42804716 |
| chrX | 43422232 | 43422282 |
| chrX | 43423497 | 43423547 |
| chrX | 43597772 | 43597822 |
| chrX | 43622446 | 43622496 |
| chr10 | 11013119 | 11013169 |
| chrX | 44176902 | 44176952 |
| chrX | 45176507 | 45176557 |
| chrX | 45673269 | 45673319 |
| chr11 | 7224408 | 7224458 |
| chrX | 46188728 | 46188778 |
| chr11 | 7224265 | 7224315 |
| chrX | 46251448 | 46251498 |
| chrX | 46301998 | 46302048 |
| chrX | 46410325 | 46410375 |
| chrX | 46452672 | 46452722 |
| chrX | 46518193 | 46518243 |
| chrX | 46767981 | 46768031 |
| chrX | 46829213 | 46829263 |
| chrX | 46828913 | 46828963 |
| chrX | 46996197 | 46996247 |
| chrX | 47486171 | 47486221 |
| chrX | 47494834 | 47494884 |
| chrX | 47512052 | 47512102 |
| chrX | 47892128 | 47892178 |
| chrX | 48485468 | 48485518 |
| chrX | 48978516 | 48978566 |
| chrX | 49616492 | 49616542 |
| chrX | 49719040 | 49719090 |
| chrX | 50318105 | 50318155 |
| chrX | 50474127 | 50474177 |
| chrX | 51800013 | 51800063 |
| chrX | 51816777 | 51816827 |
| chr11 | 7995807 | 7995857 |
| chrX | 52482628 | 52482678 |
| chrX | 54640989 | 54641039 |
| chrX | 56927997 | 56928047 |
| chrX | 63508636 | 63508686 |
| chr11 | 8331621 | 8331671 |
| chrX | 67457392 | 67457442 |
| chrX | 68459317 | 68459367 |
| chr11 | 8597269 | 8597319 |
| chrX | 69599022 | 69599072 |
| chrX | 69761106 | 69761156 |
| chrX | 71958452 | 71958502 |
| chrX | 72272216 | 72272266 |
| chrX | 72769887 | 72769937 |
| chrX | 73561274 | 73561324 |
| chrX | 74415792 | 74415842 |
| chrX | 75225019 | 75225069 |
| chrX | 75638269 | 75638319 |
| chrX | 76534776 | 76534826 |
| chrX | 78394749 | 78394799 |
| chrX | 79223071 | 79223121 |
| chrX | 79745570 | 79745620 |
| chrX | 81092602 | 81092652 |
| chrX | 84928330 | 84928380 |
| chrX | 86234639 | 86234689 |
| chrX | 86326808 | 86326858 |
| chrX | 87580189 | 87580239 |
| chrX | 88082370 | 88082420 |
| chrX | 88476179 | 88476229 |
| chrX | 88483053 | 88483103 |
| chrX | 88530558 | 88530608 |
| chrX | 89391504 | 89391554 |
| chrX | 89652156 | 89652206 |
| chrX | 91309953 | 91310003 |
| chrX | 92919015 | 92919065 |
| chrX | 93576743 | 93576793 |
| chrX | 93804980 | 93805030 |
| chrX | 96327162 | 96327212 |
| chrX | 97248819 | 97248869 |
| chrX | 98507925 | 98507975 |
| chrX | 98531857 | 98531907 |
| chrX | 98783587 | 98783637 |
| chrX | 98791584 | 98791634 |
| chrX | 100521646 | 100521696 |
| chrX | 100725697 | 100725747 |
| chrX | 101198760 | 101198810 |
| chrX | 101263683 | 101263733 |
| chrX | 101309114 | 101309164 |
| chrX | 101884039 | 101884089 |
| chrX | 101885783 | 101885833 |
| chrX | 104123657 | 104123707 |
| chrX | 104229602 | 104229652 |
| chrX | 105389184 | 105389234 |
| chrX | 105632904 | 105632954 |
| chrX | 106492964 | 106493014 |
| chr11 | 9932924 | 9932974 |
| chrX | 109550892 | 109550942 |
| chrX | 110257160 | 110257210 |
| chrX | 111031038 | 111031088 |
| chrX | 111606709 | 111606759 |
| chrX | 111711587 | 111711637 |
| chrX | 112022901 | 112022951 |
| chrX | 112549255 | 112549305 |
| chrX | 112966741 | 112966791 |
| chr11 | 10198595 | 10198645 |
| chrX | 113693462 | 113693512 |
| chrX | 114708023 | 114708073 |
| chrX | 115119612 | 115119662 |
| chrX | 116603816 | 116603866 |
| chrX | 116934192 | 116934242 |
| chrX | 117270056 | 117270106 |
| chr11 | 10468092 | 10468142 |
| chrX | 117419902 | 117419952 |
| chr11 | 10467949 | 10467999 |
| chrX | 117591018 | 117591068 |
| chrX | 118096859 | 118096909 |
| chrX | 118376855 | 118376905 |
| chrX | 119503327 | 119503377 |
| chrX | 120574243 | 120574293 |
| chrX | 121577817 | 121577867 |
| chrX | 121602113 | 121602163 |
| chrX | 121636605 | 121636655 |
| chr11 | 10715039 | 10715089 |
| chrX | 123239105 | 123239155 |
| chrX | 124249639 | 124249689 |
| chrX | 124902375 | 124902425 |
| chrX | 125725267 | 125725317 |
| chrX | 127368157 | 127368207 |
| chr11 | 10865716 | 10865766 |
| chrX | 128110094 | 128110144 |
| chr11 | 11142214 | 11142264 |
| chrX | 128774648 | 128774698 |
| chrX | 129503557 | 129503607 |
| chrX | 129510700 | 129510750 |
| chrX | 129714867 | 129714917 |
| chrX | 129747588 | 129747638 |
| chr11 | 11378035 | 11378085 |
| chrX | 131971166 | 131971216 |
| chr11 | 11509609 | 11509659 |
| chrX | 132294706 | 132294756 |
| chrX | 132368474 | 132368524 |
| chrX | 132432650 | 132432700 |
| chrX | 132790269 | 132790319 |
| chrX | 132893239 | 132893289 |
| chrX | 133436151 | 133436201 |
| chrX | 133454977 | 133455027 |
| chrX | 133512850 | 133512900 |
| chrX | 133527695 | 133527745 |
| chrX | 133547781 | 133547831 |
| chrX | 133552805 | 133552855 |
| chrX | 133681420 | 133681470 |
| chrX | 133681591 | 133681641 |
| chrX | 133702475 | 133702525 |
| chrX | 133876392 | 133876442 |
| chrX | 134676196 | 134676246 |
| chrX | 134693845 | 134693895 |
| chrX | 134789452 | 134789502 |
| chrX | 135823482 | 135823532 |
| chrX | 135943108 | 135943158 |
| chrX | 136122200 | 136122250 |
| chrX | 136679133 | 136679183 |
| chrX | 137506868 | 137506918 |
| chrX | 137783395 | 137783445 |
| chrX | 138753236 | 138753286 |
| chrX | 140728346 | 140728396 |
| chr11 | 12573813 | 12573863 |
| chrX | 143266890 | 143266940 |
| chrX | 143825204 | 143825254 |
| chrX | 146480277 | 146480327 |
| chrX | 147322032 | 147322082 |
| chrX | 147321813 | 147321863 |
| chrX | 147345673 | 147345723 |
| chrX | 147538903 | 147538953 |
| chrX | 147589546 | 147589596 |
| chrX | 149734738 | 149734788 |
| chrX | 150824952 | 150825002 |
| chrX | 151322596 | 151322646 |
| chrX | 151565512 | 151565562 |
| chrX | 151849688 | 151849738 |
| chrX | 152029152 | 152029202 |
| chrX | 152152693 | 152152743 |
| chrX | 152352044 | 152352094 |
| chrX | 152657685 | 152657735 |
| chrX | 152657818 | 152657868 |
| chr11 | 14031649 | 14031699 |
| chrX | 153295676 | 153295726 |
| chrY | 2721519 | 2721569 |
| chrY | 3305969 | 3306019 |
| chrY | 3652502 | 3652552 |
| chrY | 6144298 | 6144348 |
| chrY | 7125685 | 7125735 |
| chrY | 7812612 | 7812662 |
| chrY | 8716577 | 8716627 |
| chrY | 14172419 | 14172469 |
| chrY | 15422774 | 15422824 |
| chrY | 20979665 | 20979715 |
| chrY | 20981332 | 20981382 |
| chr11 | 14823676 | 14823726 |
| chrY | 22652474 | 22652524 |
| chrY | 27646056 | 27646106 |
| chr2 | 471654 | 471704 |
| chr2 | 797109 | 797159 |
| chr2 | 924683 | 924733 |
| chr2 | 2194334 | 2194384 |
| chr2 | 2891308 | 2891358 |
| chr2 | 2937024 | 2937074 |
| chr2 | 3292366 | 3292416 |
| chr2 | 4416961 | 4417011 |
| chr2 | 4728337 | 4728387 |
| chr2 | 4883365 | 4883415 |
| chr2 | 5087716 | 5087766 |
| chr2 | 5089608 | 5089658 |
| chr2 | 6982850 | 6982900 |
| chr2 | 7590938 | 7590988 |
| chr2 | 7590758 | 7590808 |
| chr2 | 7969849 | 7969899 |
| chr2 | 8040816 | 8040866 |
| chr2 | 8444248 | 8444298 |
| chr2 | 8603528 | 8603578 |
| chr2 | 8855284 | 8855334 |
| chr2 | 9316868 | 9316918 |
| chr2 | 9322821 | 9322871 |
| chr2 | 9822249 | 9822299 |
| chr11 | 17249662 | 17249712 |
| chr2 | 10165430 | 10165480 |
| chr2 | 10591259 | 10591309 |
| chr2 | 10592453 | 10592503 |
| chr2 | 10845829 | 10845879 |
| chr2 | 11229112 | 11229162 |
| chr2 | 11565543 | 11565593 |
| chr2 | 11623095 | 11623145 |
| chr2 | 12027931 | 12027981 |
| chr2 | 12037040 | 12037090 |
| chr2 | 12103497 | 12103547 |
| chr2 | 12227341 | 12227391 |
| chr2 | 12227279 | 12227329 |
| chr2 | 13441525 | 13441575 |
| chr2 | 13743753 | 13743803 |
| chr2 | 14721153 | 14721203 |
| chr2 | 15066592 | 15066642 |
| chr2 | 15246971 | 15247021 |
| chr2 | 16075404 | 16075454 |
| chr2 | 16076468 | 16076518 |
| chr2 | 17125977 | 17126027 |
| chr2 | 20238168 | 20238218 |
| chr2 | 20542197 | 20542247 |
| chr2 | 20738670 | 20738720 |
| chr2 | 21547264 | 21547314 |
| chr2 | 21558215 | 21558265 |
| chr2 | 22843654 | 22843704 |
| chr11 | 18195154 | 18195204 |
| chr11 | 18194906 | 18194956 |
| chr2 | 24157335 | 24157385 |
| chr2 | 24514470 | 24514520 |
| chr2 | 24598129 | 24598179 |
| chr2 | 25368365 | 25368415 |
| chr11 | 18440395 | 18440445 |
| chr11 | 18476900 | 18476950 |
| chr11 | 18476805 | 18476855 |
| chr2 | 26601514 | 26601564 |
| chr2 | 27367431 | 27367481 |
| chr2 | 27613883 | 27613933 |
| chr11 | 18636880 | 18636930 |
| chr2 | 27941470 | 27941520 |
| chr2 | 28862133 | 28862183 |
| chr2 | 29152559 | 29152609 |
| chr2 | 29288848 | 29288898 |
| chr2 | 29358772 | 29358822 |
| chr2 | 29420931 | 29420981 |
| chr2 | 29429821 | 29429871 |
| chr2 | 30293756 | 30293806 |
| chr2 | 30501804 | 30501854 |
| chr2 | 30575203 | 30575253 |
| chr2 | 30639108 | 30639158 |
| chr2 | 31019783 | 31019833 |
| chr2 | 31348051 | 31348101 |
| chr2 | 31666582 | 31666632 |
| chr2 | 31945945 | 31945995 |
| chr2 | 32250060 | 32250110 |
| chr2 | 32252011 | 32252061 |
| chr2 | 32519841 | 32519891 |
| chr2 | 32582910 | 32582960 |
| chr2 | 32955231 | 32955281 |
| chr11 | 19205174 | 19205224 |
| chr2 | 33954224 | 33954274 |
| chr2 | 33987542 | 33987592 |
| chr2 | 34274459 | 34274509 |
| chr2 | 34770583 | 34770633 |
| chr2 | 35009255 | 35009305 |
| chr2 | 35410715 | 35410765 |
| chr2 | 35988290 | 35988340 |
| chr2 | 36119281 | 36119331 |
| chr2 | 37025262 | 37025312 |
| chr11 | 19541016 | 19541066 |
| chr2 | 37866238 | 37866288 |
| chr2 | 38090377 | 38090427 |
| chr2 | 38482849 | 38482899 |
| Chr2 | 38717305 | 38717355 |
| Chr2 | 38937652 | 38937702 |
| Chr2 | 39328218 | 39328268 |
| chr2 | 41956060 | 41956110 |
| chr2 | 42143114 | 42143164 |
| chr2 | 43051909 | 43051959 |
| chr2 | 43092909 | 43092959 |
| chr2 | 43639661 | 43639711 |
| chr2 | 44234165 | 44234215 |
| chr2 | 44270329 | 44270379 |
| chr11 | 20553729 | 20553779 |
| chr10 | 11684019 | 11684069 |
| chr2 | 47418896 | 47418946 |
| chr2 | 47480090 | 47480140 |
| chr2 | 47856444 | 47856494 |
| chr2 | 47896146 | 47896196 |
| chr2 | 48354622 | 48354672 |
| chr2 | 48540004 | 48540054 |
| chr11 | 21325851 | 21325901 |
| chr11 | 21611766 | 21611816 |
| chr11 | 21798360 | 21798410 |
| chr11 | 21928843 | 21928893 |
| chr2 | 52464443 | 52464493 |
| chr2 | 52548430 | 52548480 |
| chr2 | 52740323 | 52740373 |
| chr2 | 53958434 | 53958484 |
| chr2 | 54379181 | 54379231 |
| chr2 | 54520656 | 54520706 |
| chr2 | 54989895 | 54989945 |
| chr2 | 55705088 | 55705138 |
| chr2 | 55955226 | 55955276 |
| chr2 | 56825740 | 56825790 |
| chr2 | 57647067 | 57647117 |
| chr2 | 59781378 | 59781428 |
| chr2 | 60925446 | 60925496 |
| chr2 | 62066404 | 62066454 |
| chr2 | 62475076 | 62475126 |
| chr2 | 63322487 | 63322537 |
| chr2 | 63955323 | 63955373 |
| chr2 | 64260459 | 64260509 |
| chr2 | 64263282 | 64263332 |
| chr11 | 24215940 | 24215990 |
| chr2 | 65878198 | 65878248 |
| chr2 | 66160540 | 66160590 |
| chr2 | 67824374 | 67824424 |
| chr2 | 67970764 | 67970814 |
| chr2 | 69496794 | 69496844 |
| chr2 | 69520571 | 69520621 |
| chr2 | 69941321 | 69941371 |
| chr2 | 70405449 | 70405499 |
| chr11 | 25532292 | 25532342 |
| chr11 | 25615137 | 25615187 |
| chr2 | 70795347 | 70795397 |
| chr2 | 71371359 | 71371409 |
| chr2 | 72599467 | 72599517 |
| chr2 | 73368258 | 73368308 |
| chr2 | 74336212 | 74336262 |
| chr2 | 74335998 | 74336048 |
| chr2 | 75011422 | 75011472 |
| chr2 | 75222149 | 75222199 |
| chr2 | 76030916 | 76030966 |
| chr2 | 76503875 | 76503925 |
| chr2 | 77632501 | 77632551 |
| chr2 | 78359108 | 78359158 |
| chr11 | 27271986 | 27272036 |
| chr2 | 78874346 | 78874396 |
| chr2 | 79095235 | 79095285 |
| chr2 | 79584169 | 79584219 |
| chr2 | 80031712 | 80031762 |
| chr2 | 80381322 | 80381372 |
| chr2 | 81091872 | 81091922 |
| chr2 | 84073938 | 84073988 |
| chr2 | 84075749 | 84075799 |
| chr2 | 85065486 | 85065536 |
| chr2 | 85133067 | 85133117 |
| chr2 | 85266720 | 85266770 |
| chr2 | 85268947 | 85268997 |
| chr2 | 85853318 | 85853368 |
| chr2 | 85918439 | 85918489 |
| chr11 | 27753445 | 27753495 |
| chr2 | 86019756 | 86019806 |
| chr2 | 86019989 | 86020039 |
| chr2 | 86188825 | 86188875 |
| chr2 | 86392342 | 86392392 |
| chr2 | 86393250 | 86393300 |
| chr2 | 86540573 | 86540623 |
| chr2 | 87368705 | 87368755 |
| chr2 | 88203746 | 88203796 |
| chr2 | 88619967 | 88620017 |
| chr2 | 89187161 | 89187211 |
| chr2 | 90025987 | 90026037 |
| chr2 | 91236795 | 91236845 |
| chr2 | 91321660 | 91321710 |
| chr2 | 95464653 | 95464703 |
| chr2 | 95477329 | 95477379 |
| chr11 | 29645492 | 29645542 |
| chr2 | 98437241 | 98437291 |
| chr2 | 98517720 | 98517770 |
| chr2 | 98578327 | 98578377 |
| chr2 | 100104486 | 100104536 |
| chr2 | 100129566 | 100129616 |
| chr2 | 100414391 | 100414441 |
| chr2 | 100512671 | 100512721 |
| chr2 | 100512810 | 100512860 |
| chr2 | 100516923 | 100516973 |
| chr2 | 100677865 | 100677915 |
| chr2 | 100703837 | 100703887 |
| chr2 | 100989131 | 100989181 |
| chr2 | 101677777 | 101677827 |
| chr2 | 103118135 | 103118185 |
| chr2 | 103620037 | 103620087 |
| chr2 | 104137962 | 104138012 |
| chr2 | 104381099 | 104381149 |
| chr2 | 105344981 | 105345031 |
| chr2 | 105365540 | 105365590 |
| chr2 | 105461855 | 105461905 |
| chr2 | 106538404 | 106538454 |
| chr2 | 106548380 | 106548430 |
| chr2 | 106768183 | 106768233 |
| chr2 | 106830104 | 106830154 |
| chr2 | 106929285 | 106929335 |
| chr2 | 107875411 | 107875461 |
| chr2 | 108839271 | 108839321 |
| chr2 | 109346356 | 109346406 |
| chr2 | 109699187 | 109699237 |
| chr11 | 32124136 | 32124186 |
| chr2 | 112051140 | 112051190 |
| chr2 | 112160061 | 112160111 |
| chr2 | 112651090 | 112651140 |
| chr2 | 113005174 | 113005224 |
| chr2 | 113004961 | 113005011 |
| chr2 | 113005822 | 113005872 |
| chr2 | 113111979 | 113112029 |
| chr2 | 113309565 | 113309615 |
| chr2 | 113434220 | 113434270 |
| chr2 | 113474853 | 113474903 |
| chr2 | 113527152 | 113527202 |
| chr11 | 32360279 | 32360329 |
| chr2 | 114006618 | 114006668 |
| chr2 | 114558648 | 114558698 |
| chr11 | 32438198 | 32438248 |
| chr2 | 115200059 | 115200109 |
| chr11 | 32458605 | 32458655 |
| chr11 | 32467209 | 32467259 |
| chr2 | 116810202 | 116810252 |
| chr2 | 118687749 | 118687799 |
| chr11 | 32545341 | 32545391 |
| chr11 | 32561547 | 32561597 |
| chr11 | 32642217 | 32642267 |
| chr2 | 121514478 | 121514528 |
| chr2 | 121514317 | 121514367 |
| chr2 | 121668812 | 121668862 |
| chr2 | 121926647 | 121926697 |
| chr2 | 122028884 | 122028934 |
| chr2 | 122141952 | 122142002 |
| chr2 | 122848528 | 122848578 |
| chr2 | 122848335 | 122848385 |
| chr2 | 122863244 | 122863294 |
| chr2 | 122883735 | 122883785 |
| chr2 | 123251438 | 123251488 |
| chr2 | 124336393 | 124336443 |
| chr2 | 128026928 | 128026978 |
| chr11 | 33184003 | 33184053 |
| chr2 | 128681700 | 128681750 |
| chr2 | 128683735 | 128683785 |
| chr2 | 128919997 | 128920047 |
| chr2 | 128921222 | 128921272 |
| chr2 | 129306422 | 129306472 |
| chr2 | 129378683 | 129378733 |
| chr2 | 129440205 | 129440255 |
| chr2 | 129555794 | 129555844 |
| chr2 | 130124837 | 130124887 |
| chr2 | 130362224 | 130362274 |
| chr2 | 130592697 | 130592747 |
| chr2 | 131226605 | 131226655 |
| chr2 | 131707434 | 131707484 |
| chr2 | 131717658 | 131717708 |
| chr2 | 131802956 | 131803006 |
| chr2 | 131823506 | 131823556 |
| chr2 | 131870611 | 131870661 |
| chr2 | 132938842 | 132938892 |
| chr2 | 133559628 | 133559678 |
| chr2 | 134699488 | 134699538 |
| chr2 | 135437545 | 135437595 |
| chr2 | 135453859 | 135453909 |
| chr2 | 136063678 | 136063728 |
| chr11 | 33932843 | 33932893 |
| chr2 | 136855498 | 136855548 |
| chr2 | 137115878 | 137115928 |
| chr2 | 137462657 | 137462707 |
| chr2 | 137666784 | 137666834 |
| chr11 | 34002453 | 34002503 |
| chr11 | 34003735 | 34003785 |
| chr2 | 138820462 | 138820512 |
| chr2 | 139303651 | 139303701 |
| chr2 | 139303577 | 139303627 |
| chr2 | 140817822 | 140817872 |
| chr2 | 142299359 | 142299409 |
| chr2 | 143474129 | 143474179 |
| chr2 | 144889273 | 144889323 |
| chr2 | 147000420 | 147000470 |
| chr2 | 148769141 | 148769191 |
| chr2 | 150829822 | 150829872 |
| chr2 | 151299497 | 151299547 |
| chr2 | 151657484 | 151657534 |
| chr2 | 151861321 | 151861371 |
| chr2 | 153086980 | 153087030 |
| chr2 | 153280475 | 153280525 |
| chr2 | 153644792 | 153644842 |
| chr2 | 154753315 | 154753365 |
| chr2 | 154890591 | 154890641 |
| chr2 | 155679366 | 155679416 |
| chr2 | 156069848 | 156069898 |
| chr2 | 157253723 | 157253773 |
| chr2 | 157485698 | 157485748 |
| chr2 | 158635669 | 158635719 |
| chr2 | 158995898 | 158995948 |
| chr2 | 159005532 | 159005582 |
| chr2 | 159128255 | 159128305 |
| chr2 | 161595060 | 161595110 |
| chr2 | 162546869 | 162546919 |
| chr2 | 162892086 | 162892136 |
| chr2 | 164844199 | 164844249 |
| chr2 | 165105935 | 165105985 |
| chr2 | 166093753 | 166093803 |
| chr2 | 166290295 | 166290345 |
| chr2 | 167164919 | 167164969 |
| chr2 | 167222122 | 167222172 |
| chr2 | 167697139 | 167697189 |
| chr2 | 168884380 | 168884430 |
| chr11 | 35836315 | 35836365 |
| chr2 | 169034553 | 169034603 |
| chr2 | 170755030 | 170755080 |
| chr2 | 170777492 | 170777542 |
| chr2 | 170920350 | 170920400 |
| chr2 | 171680355 | 171680405 |
| chr2 | 171939166 | 171939216 |
| chr2 | 172323989 | 172324039 |
| chr2 | 172971467 | 172971517 |
| chr2 | 173194445 | 173194495 |
| chr2 | 173267157 | 173267207 |
| chr2 | 174023621 | 174023671 |
| chr2 | 174188999 | 174189049 |
| chr2 | 174516504 | 174516554 |
| chr2 | 175333644 | 175333694 |
| chr2 | 175601131 | 175601181 |
| chr2 | 175787046 | 175787096 |
| chr11 | 36680313 | 36680363 |
| chr2 | 177804939 | 177804989 |
| chr11 | 36787977 | 36788027 |
| chr11 | 36805698 | 36805748 |
| chr11 | 36811567 | 36811617 |
| chr11 | 36811376 | 36811426 |
| chr11 | 37232568 | 37232618 |
| chr2 | 181804465 | 181804515 |
| chr2 | 181907969 | 181908019 |
| chr2 | 181977849 | 181977899 |
| chr11 | 37636994 | 37637044 |
| chr2 | 182319616 | 182319666 |
| chr2 | 182795510 | 182795560 |
| chr11 | 38053482 | 38053532 |
| chr2 | 184422215 | 184422265 |
| chr2 | 184814199 | 184814249 |
| chr2 | 185233466 | 185233516 |
| chr2 | 185762266 | 185762316 |
| chr2 | 186283894 | 186283944 |
| chr2 | 186420932 | 186420982 |
| chr2 | 188936364 | 188936414 |
| chr11 | 38491161 | 38491211 |
| chr2 | 189235924 | 189235974 |
| chr2 | 190360894 | 190360944 |
| chr11 | 39114823 | 39114873 |
| chr2 | 192975866 | 192975916 |
| chr2 | 193800706 | 193800756 |
| chr2 | 195288078 | 195288128 |
| chr2 | 195577422 | 195577472 |
| chr11 | 39330117 | 39330167 |
| chr2 | 197916861 | 197916911 |
| chr2 | 199607850 | 199607900 |
| chr2 | 199944855 | 199944905 |
| chr11 | 39662792 | 39662842 |
| chr2 | 200848951 | 200849001 |
| chr2 | 201009656 | 201009706 |
| chr2 | 201899765 | 201899815 |
| chr2 | 202123586 | 202123636 |
| chr2 | 202548247 | 202548297 |
| chr2 | 203438635 | 203438685 |
| chr2 | 203516005 | 203516055 |
| chr2 | 203754217 | 203754267 |
| chr2 | 203841478 | 203841528 |
| chr2 | 203969901 | 203969951 |
| chr2 | 204125383 | 204125433 |
| chr2 | 204295646 | 204295696 |
| chr2 | 204913811 | 204913861 |
| chr2 | 205628725 | 205628775 |
| chr2 | 206044761 | 206044811 |
| chr2 | 206885874 | 206885924 |
| chr2 | 206914016 | 206914066 |
| chr2 | 206941474 | 206941524 |
| chr2 | 206946511 | 206946561 |
| chr2 | 207049639 | 207049689 |
| chr2 | 207240404 | 207240454 |
| chr2 | 207298965 | 207299015 |
| chr2 | 207321185 | 207321235 |
| chr2 | 207324915 | 207324965 |
| chr2 | 207334707 | 207334757 |
| chr2 | 208534136 | 208534186 |
| chr11 | 41665655 | 41665705 |
| chr11 | 41867898 | 41867948 |
| chr2 | 209929341 | 209929391 |
| chr11 | 42036846 | 42036896 |
| chr11 | 42258261 | 42258311 |
| chr2 | 211554061 | 211554111 |
| chr2 | 211944927 | 211944977 |
| chr2 | 211966001 | 211966051 |
| chr2 | 211992833 | 211992883 |
| chr2 | 212139774 | 212139824 |
| chr2 | 212218929 | 212218979 |
| chr2 | 212237217 | 212237267 |
| chr2 | 212525463 | 212525513 |
| chr11 | 42807628 | 42807678 |
| chr2 | 213965712 | 213965762 |
| chr2 | 214266162 | 214266212 |
| chr2 | 214710347 | 214710397 |
| chr2 | 216896575 | 216896625 |
| chr2 | 217674089 | 217674139 |
| chr2 | 218823906 | 218823956 |
| chr2 | 220104294 | 220104344 |
| chr2 | 220345023 | 220345073 |
| chr11 | 43858049 | 43858099 |
| chr11 | 43863203 | 43863253 |
| chr2 | 220709870 | 220709920 |
| chr2 | 220998967 | 220999017 |
| chr2 | 221389386 | 221389436 |
| chr2 | 222409950 | 222410000 |
| chr2 | 222983630 | 222983680 |
| chr2 | 223814407 | 223814457 |
| chr2 | 223890228 | 223890278 |
| chr2 | 224369830 | 224369880 |
| chr2 | 224553789 | 224553839 |
| chr2 | 224937948 | 224937998 |
| chr2 | 225436272 | 225436322 |
| chr2 | 225669938 | 225669988 |
| chr2 | 225671232 | 225671282 |
| chr2 | 225686623 | 225686673 |
| chr2 | 225818525 | 225818575 |
| chr2 | 226805401 | 226805451 |
| chr2 | 227293428 | 227293478 |
| chr2 | 228727285 | 228727335 |
| chr2 | 228872995 | 228873045 |
| chr2 | 229435141 | 229435191 |
| chr2 | 229551575 | 229551625 |
| chr2 | 230461139 | 230461189 |
| chr11 | 44472655 | 44472705 |
| chr2 | 231240337 | 231240387 |
| chr2 | 231240075 | 231240125 |
| chr2 | 231303416 | 231303466 |
| chr2 | 231470809 | 231470859 |
| chr2 | 231613616 | 231613666 |
| chr2 | 232353300 | 232353350 |
| chr2 | 232490643 | 232490693 |
| chr2 | 232493549 | 232493599 |
| chr2 | 232614627 | 232614677 |
| chr2 | 233000028 | 233000078 |
| chr2 | 233460964 | 233461014 |
| chr2 | 233657661 | 233657711 |
| chr11 | 44958979 | 44959029 |
| chr2 | 234980103 | 234980153 |
| chr11 | 45097071 | 45097121 |
| chr2 | 235749143 | 235749193 |
| chr2 | 235812172 | 235812222 |
| chr2 | 236190000 | 236190050 |
| chr2 | 236259198 | 236259248 |
| chr2 | 236259024 | 236259074 |
| chr2 | 236888280 | 236888330 |
| chr11 | 45364619 | 45364669 |
| chr11 | 45431232 | 45431282 |
| chr2 | 237602498 | 237602548 |
| chr2 | 237800571 | 237800621 |
| chr11 | 45570313 | 45570363 |
| chr2 | 238276865 | 238276915 |
| chr2 | 238378088 | 238378138 |
| chr2 | 238377920 | 238377970 |
| chr2 | 238641729 | 238641779 |
| chr2 | 238641668 | 238641718 |
| chr11 | 45717457 | 45717507 |
| chr2 | 238710706 | 238710756 |
| chr2 | 238719475 | 238719525 |
| chr2 | 239318428 | 239318478 |
| chr2 | 239420221 | 239420271 |
| chr2 | 239493708 | 239493758 |
| chr2 | 239752659 | 239752709 |
| chr2 | 239766590 | 239766640 |
| chr2 | 239781973 | 239782023 |
| chr2 | 239837832 | 239837882 |
| chr2 | 240077422 | 240077472 |
| chr2 | 240546021 | 240546071 |
| chr2 | 240601790 | 240601840 |
| chr2 | 240711975 | 240712025 |
| chr2 | 241153007 | 241153057 |
| chr2 | 241152898 | 241152948 |
| chr2 | 241169470 | 241169520 |
| chr2 | 241173387 | 241173437 |
| chr2 | 241222096 | 241222146 |
| chr2 | 241364100 | 241364150 |
| chr2 | 241401215 | 241401265 |
| chr2 | 241402681 | 241402731 |
| chr2 | 241738964 | 241739014 |
| chr2 | 241852900 | 241852950 |
| chr2 | 241876510 | 241876560 |
| chr2 | 241947266 | 241947316 |
| chr2 | 241969920 | 241969970 |
| chr2 | 242113098 | 242113148 |
| chr2 | 242278366 | 242278416 |
| chr2 | 242279651 | 242279701 |
| chr2 | 242794193 | 242794243 |
| chr2 | 242834373 | 242834423 |
| chr2 | 243020202 | 243020252 |
| chr2 | 243279464 | 243279514 |
| chr3 | 636641 | 636691 |
| chr3 | 2154462 | 2154512 |
| chr3 | 2241422 | 2241472 |
| chr3 | 3259320 | 3259370 |
| chr3 | 3847958 | 3848008 |
| chr3 | 6624254 | 6624304 |
| chr11 | 46932010 | 46932060 |
| chr3 | 11113330 | 11113380 |
| chr3 | 11136084 | 11136134 |
| chr11 | 47026052 | 47026102 |
| chr3 | 11478672 | 11478722 |
| chr3 | 11992030 | 11992080 |
| chr3 | 12728266 | 12728316 |
| chr3 | 12748811 | 12748861 |
| chr3 | 12901733 | 12901783 |
| chr3 | 13183607 | 13183657 |
| chr3 | 13183439 | 13183489 |
| chr3 | 13211184 | 13211234 |
| chr3 | 13790933 | 13790983 |
| chr3 | 14998439 | 14998489 |
| chr3 | 15126558 | 15126608 |
| chr3 | 15126499 | 15126549 |
| chr3 | 15149900 | 15149950 |
| chr3 | 15304330 | 15304380 |
| chr3 | 15304177 | 15304227 |
| chr3 | 16166469 | 16166519 |
| chr3 | 16362141 | 16362191 |
| chr3 | 16491880 | 16491930 |
| chr3 | 16491653 | 16491703 |
| chr3 | 16573343 | 16573393 |
| chr3 | 16720418 | 16720468 |
| chr3 | 17765611 | 17765661 |
| chr3 | 18093575 | 18093625 |
| chr3 | 18274759 | 18274809 |
| chr3 | 18985701 | 18985751 |
| chr3 | 19906213 | 19906263 |
| chr3 | 20604352 | 20604402 |
| chr11 | 47416833 | 47416883 |
| chr3 | 21913841 | 21913891 |
| chr3 | 22159278 | 22159328 |
| chr3 | 22392665 | 22392715 |
| chr3 | 22729631 | 22729681 |
| chr3 | 23086628 | 23086678 |
| chr3 | 23289221 | 23289271 |
| chr3 | 23452238 | 23452288 |
| chr3 | 23945061 | 23945111 |
| chr3 | 25589932 | 25589982 |
| chr3 | 26702818 | 26702868 |
| chr3 | 27525500 | 27525550 |
| chr3 | 27557442 | 27557492 |
| chr3 | 27577270 | 27577320 |
| chr3 | 29573208 | 29573258 |
| chr11 | 47583890 | 47583940 |
| chr3 | 30597679 | 30597729 |
| chr3 | 30653559 | 30653609 |
| chr3 | 30698142 | 30698192 |
| chr3 | 30698023 | 30698073 |
| chr3 | 30999988 | 31000038 |
| chr3 | 31130942 | 31130992 |
| chr3 | 31250206 | 31250256 |
| chr3 | 31378935 | 31378985 |
| chr3 | 32275028 | 32275078 |
| chr3 | 32274925 | 32274975 |
| chr3 | 32664612 | 32664662 |
| chr3 | 33093742 | 33093792 |
| chr3 | 33158655 | 33158705 |
| chr3 | 33158426 | 33158476 |
| chr3 | 33781374 | 33781424 |
| chr3 | 34655753 | 34655803 |
| chr3 | 34725721 | 34725771 |
| chr3 | 34926802 | 34926852 |
| chr3 | 35146376 | 35146426 |
| chr3 | 35492659 | 35492709 |
| chr3 | 35621701 | 35621751 |
| chr3 | 36827118 | 36827168 |
| chr3 | 36892019 | 36892069 |
| chr3 | 36906844 | 36906894 |
| chr3 | 37696737 | 37696787 |
| chr3 | 39504671 | 39504721 |
| chr3 | 39772166 | 39772216 |
| chr3 | 39968701 | 39968751 |
| chr3 | 40242696 | 40242746 |
| chr3 | 41307227 | 41307277 |
| chr3 | 41966143 | 41966193 |
| chr3 | 42586294 | 42586344 |
| chr3 | 43121963 | 43122013 |
| chr3 | 43211473 | 43211523 |
| chr3 | 43646441 | 43646491 |
| chr3 | 43860413 | 43860463 |
| chr3 | 44148089 | 44148139 |
| chr3 | 44168386 | 44168436 |
| chr3 | 44174416 | 44174466 |
| chr3 | 44415431 | 44415481 |
| chr3 | 44435106 | 44435156 |
| chr3 | 45169824 | 45169874 |
| chr3 | 45208409 | 45208459 |
| chr3 | 45339042 | 45339092 |
| chr11 | 49070741 | 49070791 |
| chr10 | 12483056 | 12483106 |
| chr3 | 46283027 | 46283077 |
| chr3 | 46490915 | 46490965 |
| chr3 | 46518320 | 46518370 |
| chr3 | 46560705 | 46560755 |
| chr3 | 46818060 | 46818110 |
| chr3 | 46837350 | 46837400 |
| chr3 | 48006886 | 48006936 |
| chr3 | 48229593 | 48229643 |
| chr3 | 48232390 | 48232440 |
| chr3 | 48295054 | 48295104 |
| chr3 | 48510709 | 48510759 |
| chr3 | 48847357 | 48847407 |
| chr11 | 54806468 | 54806518 |
| chr3 | 50079001 | 50079051 |
| chr3 | 50997473 | 50997523 |
| chr3 | 51795554 | 51795604 |
| chr3 | 51814096 | 51814146 |
| chr3 | 51861954 | 51862004 |
| chr3 | 52373953 | 52374003 |
| chr3 | 52374334 | 52374384 |
| chr11 | 56165381 | 56165431 |
| chr11 | 56183605 | 56183655 |
| chr3 | 53270082 | 53270132 |
| chr3 | 53409110 | 53409160 |
| chr3 | 53416519 | 53416569 |
| chr3 | 55791797 | 55791847 |
| chr3 | 57058513 | 57058563 |
| chr3 | 57715997 | 57716047 |
| chr3 | 58659508 | 58659558 |
| chr3 | 62465882 | 62465932 |
| chr3 | 63068263 | 63068313 |
| chr3 | 63618129 | 63618179 |
| chr3 | 66679032 | 66679082 |
| chr3 | 67063196 | 67063246 |
| chr3 | 68470120 | 68470170 |
| chr3 | 69599178 | 69599228 |
| chr11 | 57543049 | 57543099 |
| chr3 | 71859392 | 71859442 |
| chr3 | 71949171 | 71949221 |
| chr3 | 72018406 | 72018456 |
| chr3 | 72058973 | 72059023 |
| chr3 | 72158860 | 72158910 |
| chr3 | 72231466 | 72231516 |
| chr3 | 72828268 | 72828318 |
| chr3 | 72884683 | 72884733 |
| chr3 | 72884625 | 72884675 |
| chr3 | 72885516 | 72885566 |
| chr3 | 72939134 | 72939184 |
| chr3 | 73153516 | 73153566 |
| chr3 | 74516329 | 74516379 |
| chr3 | 74718463 | 74718513 |
| chr3 | 74862944 | 74862994 |
| chr3 | 76173883 | 76173933 |
| chr3 | 76173626 | 76173676 |
| chr3 | 76594533 | 76594583 |
| chr3 | 77645541 | 77645591 |
| chr11 | 58237769 | 58237819 |
| chr3 | 78079437 | 78079487 |
| chr11 | 58306356 | 58306406 |
| chr3 | 80796659 | 80796709 |
| chr3 | 81050160 | 81050210 |
| chr3 | 81205847 | 81205897 |
| chr3 | 81220660 | 81220710 |
| chr3 | 82351603 | 82351653 |
| chr3 | 82856433 | 82856483 |
| chr3 | 83273813 | 83273863 |
| chr3 | 83287373 | 83287423 |
| chr3 | 83704488 | 83704538 |
| chr3 | 83845963 | 83846013 |
| chr3 | 84560278 | 84560328 |
| chr3 | 85318069 | 85318119 |
| chr3 | 85424472 | 85424522 |
| chr3 | 86306169 | 86306219 |
| chr3 | 86348934 | 86348984 |
| chr3 | 87345355 | 87345405 |
| chr3 | 87346261 | 87346311 |
| chr3 | 88711279 | 88711329 |
| chr3 | 96309745 | 96309795 |
| chr3 | 96404726 | 96404776 |
| chr3 | 96437407 | 96437457 |
| chr3 | 97591732 | 97591782 |
| chr11 | 59139751 | 59139801 |
| chr3 | 100637825 | 100637875 |
| chr3 | 100637658 | 100637708 |
| chr3 | 101289390 | 101289440 |
| chr3 | 102658141 | 102658191 |
| chr3 | 105532960 | 105533010 |
| chr3 | 105605117 | 105605167 |
| chr3 | 106151957 | 106152007 |
| chr3 | 106644031 | 106644081 |
| chr3 | 106648615 | 106648665 |
| chr3 | 106881638 | 106881688 |
| chr3 | 107379342 | 107379392 |
| chr3 | 107690373 | 107690423 |
| chr3 | 108172717 | 108172767 |
| chr3 | 108288719 | 108288769 |
| chr3 | 108288519 | 108288569 |
| chr3 | 108807770 | 108807820 |
| chr3 | 109154382 | 109154432 |
| chr3 | 109781633 | 109781683 |
| chr3 | 110398313 | 110398363 |
| chr11 | 59918930 | 59918980 |
| chr3 | 110614107 | 110614157 |
| chr3 | 110668288 | 110668338 |
| chr3 | 114075108 | 114075158 |
| chr3 | 114880044 | 114880094 |
| chr3 | 115038221 | 115038271 |
| chr3 | 115181839 | 115181889 |
| chr3 | 115976622 | 115976672 |
| chr3 | 117234435 | 117234485 |
| chr11 | 60328225 | 60328275 |
| chr3 | 118031464 | 118031514 |
| chr3 | 118527736 | 118527786 |
| chr11 | 60349829 | 60349879 |
| chr3 | 119403971 | 119404021 |
| chr3 | 119564017 | 119564067 |
| chr3 | 120219169 | 120219219 |
| chr3 | 120652007 | 120652057 |
| chr10 | 12738935 | 12738985 |
| chr3 | 121262307 | 121262357 |
| chr3 | 121500602 | 121500652 |
| chr3 | 121567470 | 121567520 |
| chr3 | 124374986 | 124375036 |
| chr3 | 124459135 | 124459185 |
| chr3 | 124458995 | 124459045 |
| chr3 | 125003864 | 125003914 |
| chr3 | 127076108 | 127076158 |
| chr3 | 127174083 | 127174133 |
| chr3 | 127432145 | 127432195 |
| chr3 | 127873945 | 127873995 |
| chr3 | 128217716 | 128217766 |
| chr3 | 128509739 | 128509789 |
| chr3 | 128580101 | 128580151 |
| chr3 | 128580276 | 128580326 |
| chr3 | 128633027 | 128633077 |
| chr3 | 129447755 | 129447805 |
| chr3 | 129759561 | 129759611 |
| chr10 | 12792749 | 12792799 |
| chr3 | 131146964 | 131147014 |
| chr3 | 131438272 | 131438322 |
| chr3 | 131539530 | 131539580 |
| chr3 | 131876394 | 131876444 |
| chr11 | 61041191 | 61041241 |
| chr3 | 132636516 | 132636566 |
| chr3 | 132760317 | 132760367 |
| chr3 | 134541551 | 134541601 |
| chr3 | 134636906 | 134636956 |
| chr3 | 134708691 | 134708741 |
| chr3 | 135363630 | 135363680 |
| chr3 | 135687854 | 135687904 |
| chr3 | 136092810 | 136092860 |
| chr3 | 137276376 | 137276426 |
| chr3 | 137548495 | 137548545 |
| chr3 | 137619646 | 137619696 |
| chr3 | 137841199 | 137841249 |
| chr3 | 139926130 | 139926180 |
| chr3 | 139961212 | 139961262 |
| chr3 | 140795956 | 140796006 |
| chr3 | 141666931 | 141666981 |
| chr3 | 141860311 | 141860361 |
| chr3 | 141881935 | 141881985 |
| chr3 | 141950363 | 141950413 |
| chr3 | 142134853 | 142134903 |
| chr3 | 142352724 | 142352774 |
| chr3 | 143162507 | 143162557 |
| chr3 | 143268181 | 143268231 |
| chr3 | 143281307 | 143281357 |
| chr3 | 143932187 | 143932237 |
| chr3 | 144124545 | 144124595 |
| chr3 | 144297656 | 144297706 |
| chr3 | 144637003 | 144637053 |
| chr3 | 145220172 | 145220222 |
| chr3 | 145865320 | 145865370 |
| chr3 | 148009254 | 148009304 |
| chr3 | 148958405 | 148958455 |
| chr3 | 149220122 | 149220172 |
| chr3 | 149319529 | 149319579 |
| chr3 | 149709193 | 149709243 |
| chr3 | 149710458 | 149710508 |
| chr3 | 149793482 | 149793532 |
| chr3 | 149860602 | 149860652 |
| chr11 | 61443238 | 61443288 |
| chr3 | 152966638 | 152966688 |
| chr3 | 153957555 | 153957605 |
| chr3 | 154031575 | 154031625 |
| chr3 | 154745129 | 154745179 |
| chr3 | 154962170 | 154962220 |
| chr3 | 156037804 | 156037854 |
| chr3 | 157121064 | 157121114 |
| chr3 | 157144049 | 157144099 |
| chr3 | 157971760 | 157971810 |
| chr3 | 160592849 | 160592899 |
| chr3 | 161214134 | 161214184 |
| chr3 | 162921952 | 162922002 |
| chr3 | 163456801 | 163456851 |
| chr3 | 163628250 | 163628300 |
| chr3 | 164025836 | 164025886 |
| chr3 | 165035022 | 165035072 |
| chr3 | 165159473 | 165159523 |
| chr3 | 166336650 | 166336700 |
| chr3 | 166393703 | 166393753 |
| chr3 | 166484099 | 166484149 |
| chr3 | 166943374 | 166943424 |
| chr3 | 169013548 | 169013598 |
| chr3 | 169820168 | 169820218 |
| chr3 | 170102108 | 170102158 |
| chr3 | 170541269 | 170541319 |
| chr3 | 170541081 | 170541131 |
| chr3 | 171035319 | 171035369 |
| chr3 | 171035051 | 171035101 |
| chr3 | 171420074 | 171420124 |
| chr3 | 172577741 | 172577791 |
| chr3 | 172952261 | 172952311 |
| chr3 | 173038076 | 173038126 |
| chr3 | 173655590 | 173655640 |
| chr3 | 174491165 | 174491215 |
| chr3 | 175270817 | 175270867 |
| chr3 | 175272319 | 175272369 |
| chr3 | 176021815 | 176021865 |
| chr3 | 176609269 | 176609319 |
| chr3 | 176732223 | 176732273 |
| chr3 | 177251217 | 177251267 |
| chr3 | 177252690 | 177252740 |
| chr3 | 177259309 | 177259359 |
| chr3 | 178307116 | 178307166 |
| chr3 | 178309238 | 178309288 |
| chr3 | 178342233 | 178342283 |
| chr3 | 178363070 | 178363120 |
| chr3 | 178599485 | 178599535 |
| chr3 | 180135184 | 180135234 |
| chr3 | 181796123 | 181796173 |
| chr3 | 183115306 | 183115356 |
| chr10 | 12947565 | 12947615 |
| chr3 | 184226537 | 184226587 |
| chr3 | 184585549 | 184585599 |
| chr3 | 184610520 | 184610570 |
| chr3 | 185450976 | 185451026 |
| chr3 | 185488418 | 185488468 |
| chr3 | 185503717 | 185503767 |
| chr3 | 185581029 | 185581079 |
| chr3 | 186134967 | 186135017 |
| chr3 | 186919983 | 186920033 |
| chr3 | 187851359 | 187851409 |
| chr3 | 187877950 | 187878000 |
| chr3 | 188087926 | 188087976 |
| chr3 | 188148071 | 188148121 |
| chr3 | 188263334 | 188263384 |
| chr3 | 188417573 | 188417623 |
| chr11 | 63109115 | 63109165 |
| chr3 | 190403197 | 190403247 |
| chr3 | 191084870 | 191084920 |
| chr3 | 191203153 | 191203203 |
| chr3 | 191567923 | 191567973 |
| chr3 | 191789601 | 191789651 |
| chr3 | 192163079 | 192163129 |
| chr3 | 192273451 | 192273501 |
| chr3 | 192347828 | 192347878 |
| chr3 | 192959356 | 192959406 |
| chr3 | 193161935 | 193161985 |
| chr3 | 194057014 | 194057064 |
| chr3 | 194615257 | 194615307 |
| chr3 | 194898084 | 194898134 |
| chr3 | 195027278 | 195027328 |
| chr3 | 195306735 | 195306785 |
| chr3 | 195810704 | 195810754 |
| chr3 | 195892864 | 195892914 |
| chr3 | 196025398 | 196025448 |
| chr3 | 196155953 | 196156003 |
| chr3 | 196496693 | 196496743 |
| chr3 | 196902945 | 196902995 |
| chr3 | 196995464 | 196995514 |
| chr3 | 197057427 | 197057477 |
| chr3 | 197163946 | 197163996 |
| chr3 | 197514149 | 197514199 |
| chr3 | 197658103 | 197658153 |
| chr3 | 197681298 | 197681348 |
| chr3 | 198023908 | 198023958 |
| chr3 | 198645087 | 198645137 |
| chr3 | 198646440 | 198646490 |
| chr3 | 198647379 | 198647429 |
| chr3 | 198694503 | 198694553 |
| chr3 | 198959631 | 198959681 |
| chr3 | 198962930 | 198962980 |
| chr3 | 199010877 | 199010927 |
| chr3 | 199011098 | 199011148 |
| chr4 | 262844 | 262894 |
| chr4 | 439031 | 439081 |
| chr4 | 683393 | 683443 |
| chr4 | 966070 | 966120 |
| chr4 | 1254919 | 1254969 |
| chr4 | 1289256 | 1289306 |
| chr4 | 1356516 | 1356566 |
| chr4 | 2000184 | 2000234 |
| chr4 | 2773936 | 2773986 |
| chr4 | 3347791 | 3347841 |
| chr4 | 3354055 | 3354105 |
| chr4 | 3353869 | 3353919 |
| chr4 | 3580854 | 3580904 |
| chr4 | 3606791 | 3606841 |
| chr4 | 3746624 | 3746674 |
| chr4 | 3775205 | 3775255 |
| chr4 | 3777817 | 3777867 |
| chr4 | 4320893 | 4320943 |
| chr4 | 4323195 | 4323245 |
| chr4 | 4595318 | 4595368 |
| chr4 | 5053098 | 5053148 |
| chr4 | 5331240 | 5331290 |
| chr4 | 5692963 | 5693013 |
| chr4 | 5731178 | 5731228 |
| chr4 | 5744178 | 5744228 |
| chr4 | 5744025 | 5744075 |
| chr4 | 5871224 | 5871274 |
| chr4 | 5906082 | 5906132 |
| chr4 | 5935984 | 5936034 |
| chr11 | 63942753 | 63942803 |
| chr4 | 6083370 | 6083420 |
| chr4 | 6083158 | 6083208 |
| chr4 | 6084408 | 6084458 |
| chr4 | 6173853 | 6173903 |
| chr4 | 6203727 | 6203777 |
| chr11 | 63992391 | 63992441 |
| chr4 | 6375852 | 6375902 |
| chr11 | 63992749 | 63992799 |
| chr11 | 63995079 | 63995129 |
| chr11 | 63997374 | 63997424 |
| chr11 | 63999493 | 63999543 |
| chr4 | 6621795 | 6621845 |
| chr11 | 64045589 | 64045639 |
| chr4 | 6833552 | 6833602 |
| chr11 | 64064471 | 64064521 |
| chr4 | 6943473 | 6943523 |
| chr4 | 7149474 | 7149524 |
| chr4 | 7213137 | 7213187 |
| chr4 | 7221949 | 7221999 |
| chr4 | 7224898 | 7224948 |
| chr11 | 64114644 | 64114694 |
| chr4 | 7575155 | 7575205 |
| chr11 | 64141813 | 64141863 |
| chr4 | 7706769 | 7706819 |
| chr4 | 7919749 | 7919799 |
| chr4 | 8402586 | 8402636 |
| chr4 | 8455416 | 8455466 |
| chr4 | 8704014 | 8704064 |
| chr4 | 8743364 | 8743414 |
| chr4 | 8795787 | 8795837 |
| chr4 | 8904501 | 8904551 |
| chr4 | 9625483 | 9625533 |
| chr4 | 9625260 | 9625310 |
| chr4 | 9897882 | 9897932 |
| chr4 | 10617774 | 10617824 |
| chr4 | 12001718 | 12001768 |
| chr4 | 12377656 | 12377706 |
| chr4 | 12951194 | 12951244 |
| chr4 | 13211129 | 13211179 |
| chr11 | 64364847 | 64364897 |
| chr4 | 13406821 | 13406871 |
| chr4 | 13406756 | 13406806 |
| chr4 | 13463351 | 13463401 |
| chr4 | 13768057 | 13768107 |
| chr4 | 13979519 | 13979569 |
| chr4 | 14307163 | 14307213 |
| chr4 | 14926482 | 14926532 |
| chr4 | 16566743 | 16566793 |
| chr4 | 16664238 | 16664288 |
| chr4 | 17461938 | 17461988 |
| chr4 | 17498072 | 17498122 |
| chr4 | 17676139 | 17676189 |
| chr4 | 17891457 | 17891507 |
| chr4 | 18797556 | 18797606 |
| chr4 | 20474958 | 20475008 |
| chr4 | 22351979 | 22352029 |
| chr4 | 22779544 | 22779594 |
| chr4 | 22863337 | 22863387 |
| chr4 | 23072794 | 23072844 |
| chr4 | 23665149 | 23665199 |
| chr4 | 24153282 | 24153332 |
| chr4 | 24360761 | 24360811 |
| chr4 | 24364221 | 24364271 |
| chr4 | 24422495 | 24422545 |
| chr4 | 24525493 | 24525543 |
| chr4 | 24718269 | 24718319 |
| chr4 | 25053460 | 25053510 |
| chr4 | 25706985 | 25707035 |
| chr4 | 26642946 | 26642996 |
| chr4 | 27278603 | 27278653 |
| chr4 | 27309673 | 27309723 |
| chr4 | 28935983 | 28936033 |
| chr4 | 29142097 | 29142147 |
| chr4 | 29509997 | 29510047 |
| chr4 | 29653120 | 29653170 |
| chr11 | 64704210 | 64704260 |
| chr4 | 32681056 | 32681106 |
| chr4 | 32868244 | 32868294 |
| chr4 | 33257601 | 33257651 |
| chr4 | 33927045 | 33927095 |
| chr4 | 34107951 | 34108001 |
| chr4 | 34395984 | 34396034 |
| chr4 | 34593456 | 34593506 |
| chr4 | 35477560 | 35477610 |
| chr4 | 36307619 | 36307669 |
| chr4 | 36361613 | 36361663 |
| chr4 | 36868320 | 36868370 |
| chr4 | 38479178 | 38479228 |
| chr4 | 39034261 | 39034311 |
| chr4 | 39172103 | 39172153 |
| chr4 | 39402679 | 39402729 |
| chr4 | 39562913 | 39562963 |
| chr11 | 64872853 | 64872903 |
| chr4 | 39917653 | 39917703 |
| chr4 | 40150221 | 40150271 |
| chr4 | 40778487 | 40778537 |
| chr4 | 40800479 | 40800529 |
| chr10 | 13358890 | 13358940 |
| chr4 | 42112098 | 42112148 |
| chr4 | 42159278 | 42159328 |
| chr11 | 65000933 | 65000983 |
| chr11 | 65003011 | 65003061 |
| chr4 | 45082866 | 45082916 |
| chr4 | 45511568 | 45511618 |
| chr4 | 46066583 | 46066633 |
| chr4 | 46918627 | 46918677 |
| chr10 | 13367740 | 13367790 |
| chr4 | 47650448 | 47650498 |
| chr4 | 47746895 | 47746945 |
| chr4 | 47844535 | 47844585 |
| chr4 | 48219007 | 48219057 |
| chr4 | 48246533 | 48246583 |
| chr4 | 48850460 | 48850510 |
| chr4 | 53035587 | 53035637 |
| chr4 | 54017639 | 54017689 |
| chr4 | 55185003 | 55185053 |
| chr4 | 55291213 | 55291263 |
| chr4 | 55613038 | 55613088 |
| chr4 | 56352967 | 56353017 |
| chr11 | 65213837 | 65213887 |
| chr11 | 65214358 | 65214408 |
| chr4 | 58884469 | 58884519 |
| chr4 | 59153299 | 59153349 |
| chr11 | 65250440 | 65250490 |
| chr4 | 59517785 | 59517835 |
| chr4 | 60345528 | 60345578 |
| chr4 | 60833491 | 60833541 |
| chr4 | 61366061 | 61366111 |
| chr4 | 61487178 | 61487228 |
| chr4 | 61729818 | 61729868 |
| chr4 | 62297442 | 62297492 |
| chr4 | 62297235 | 62297285 |
| chr4 | 63721674 | 63721724 |
| chr4 | 63915993 | 63916043 |
| chr4 | 64886141 | 64886191 |
| chr4 | 64908564 | 64908614 |
| chr4 | 67564734 | 67564784 |
| chr4 | 67648786 | 67648836 |
| chr4 | 68022104 | 68022154 |
| chr4 | 68421004 | 68421054 |
| chr4 | 68647864 | 68647914 |
| chr4 | 70977706 | 70977756 |
| chr4 | 73020500 | 73020550 |
| chr4 | 73345511 | 73345561 |
| chr4 | 73458954 | 73459004 |
| chr4 | 74617968 | 74618018 |
| chr4 | 74905738 | 74905788 |
| chr4 | 77238673 | 77238723 |
| chr4 | 78996868 | 78996918 |
| chr4 | 79084746 | 79084796 |
| chr4 | 80812226 | 80812276 |
| chr4 | 81097952 | 81098002 |
| chr4 | 81207097 | 81207147 |
| chr4 | 82109015 | 82109065 |
| chr4 | 83293080 | 83293130 |
| chr4 | 83464640 | 83464690 |
| chr4 | 83607792 | 83607842 |
| chr4 | 83776071 | 83776121 |
| chr4 | 83796367 | 83796417 |
| chr4 | 84369941 | 84369991 |
| chr4 | 84739870 | 84739920 |
| chr4 | 84908468 | 84908518 |
| chr4 | 86920983 | 86921033 |
| chr4 | 87176510 | 87176560 |
| chr4 | 87992869 | 87992919 |
| chr4 | 88159012 | 88159062 |
| chr4 | 88708640 | 88708690 |
| chr4 | 89006727 | 89006777 |
| chr4 | 89147477 | 89147527 |
| chr4 | 89200957 | 89201007 |
| chr4 | 89200754 | 89200804 |
| chr4 | 89881624 | 89881674 |
| chr4 | 89912622 | 89912672 |
| chr4 | 91520498 | 91520548 |
| chr4 | 91637694 | 91637744 |
| chr4 | 93357607 | 93357657 |
| chr4 | 93365146 | 93365196 |
| chr4 | 94193461 | 94193511 |
| chr4 | 95536618 | 95536668 |
| chr4 | 95621406 | 95621456 |
| chr4 | 97374024 | 97374074 |
| chr4 | 97650882 | 97650932 |
| chr4 | 98350549 | 98350599 |
| chr4 | 98431277 | 98431327 |
| chr4 | 99579099 | 99579149 |
| chr4 | 99692300 | 99692350 |
| chr4 | 99900446 | 99900496 |
| chr4 | 99961842 | 99961892 |
| chr4 | 101013673 | 101013723 |
| chr4 | 101047910 | 101047960 |
| chr4 | 102542122 | 102542172 |
| chr4 | 102740584 | 102740634 |
| chr4 | 102996809 | 102996859 |
| chr4 | 104125960 | 104126010 |
| chr4 | 104224332 | 104224382 |
| chr4 | 104926179 | 104926229 |
| chr4 | 106141713 | 106141763 |
| chr4 | 106178019 | 106178069 |
| chr4 | 106234021 | 106234071 |
| chr4 | 106423606 | 106423656 |
| chr4 | 106855025 | 106855075 |
| chr4 | 110633745 | 110633795 |
| chr4 | 110740835 | 110740885 |
| chr4 | 110773598 | 110773648 |
| chr4 | 111184566 | 111184616 |
| chr4 | 111185024 | 111185074 |
| chr4 | 111594382 | 111594432 |
| chr4 | 111637872 | 111637922 |
| chr4 | 112556663 | 112556713 |
| chr4 | 113189812 | 113189862 |
| chr4 | 113576828 | 113576878 |
| chr4 | 115894994 | 115895044 |
| chr4 | 116308807 | 116308857 |
| chr4 | 117018984 | 117019034 |
| chr4 | 117308835 | 117308885 |
| chr4 | 117912241 | 117912291 |
| chr4 | 119971501 | 119971551 |
| chr4 | 120208011 | 120208061 |
| chr4 | 122028495 | 122028545 |
| chr4 | 122429264 | 122429314 |
| chr4 | 123021598 | 123021648 |
| chr4 | 123078212 | 123078262 |
| chr4 | 123490029 | 123490079 |
| chr4 | 125498823 | 125498873 |
| chr4 | 127195479 | 127195529 |
| chr4 | 127871272 | 127871322 |
| chr4 | 129808057 | 129808107 |
| chr4 | 129807924 | 129807974 |
| chr4 | 129845885 | 129845935 |
| chr4 | 129890690 | 129890740 |
| chr4 | 129892698 | 129892748 |
| chr4 | 129943795 | 129943845 |
| chr4 | 130089597 | 130089647 |
| chr11 | 66511904 | 66511954 |
| chr4 | 130924849 | 130924899 |
| chr4 | 132131127 | 132131177 |
| chr4 | 132206823 | 132206873 |
| chr4 | 132678146 | 132678196 |
| chr4 | 132770450 | 132770500 |
| chr4 | 133470625 | 133470675 |
| chr4 | 133551944 | 133551994 |
| chr4 | 133902622 | 133902672 |
| chr11 | 66583017 | 66583067 |
| chr4 | 134619222 | 134619272 |
| chr4 | 134620803 | 134620853 |
| chr4 | 134836365 | 134836415 |
| chr11 | 66592544 | 66592594 |
| chr4 | 135529961 | 135530011 |
| chr4 | 135587729 | 135587779 |
| chr4 | 136408291 | 136408341 |
| chr4 | 137017604 | 137017654 |
| chr4 | 137084618 | 137084668 |
| chr4 | 138723448 | 138723498 |
| chr4 | 138789336 | 138789386 |
| chr4 | 139626989 | 139627039 |
| chr4 | 140941457 | 140941507 |
| chr4 | 140996949 | 140996999 |
| chr4 | 141358732 | 141358782 |
| chr4 | 141708642 | 141708692 |
| chr4 | 142427875 | 142427925 |
| chr4 | 142706444 | 142706494 |
| chr4 | 142706241 | 142706291 |
| chr4 | 143394751 | 143394801 |
| chr4 | 143713168 | 143713218 |
| chr4 | 143837340 | 143837390 |
| chr4 | 144016064 | 144016114 |
| chr4 | 144015864 | 144015914 |
| chr4 | 144775597 | 144775647 |
| chr4 | 145343719 | 145343769 |
| chr4 | 145464367 | 145464417 |
| chr4 | 145926100 | 145926150 |
| chr4 | 146139065 | 146139115 |
| chr4 | 148289249 | 148289299 |
| chr4 | 149093566 | 149093616 |
| chr4 | 150163435 | 150163485 |
| chr4 | 150521451 | 150521501 |
| chr4 | 150521148 | 150521198 |
| chr4 | 150808034 | 150808084 |
| chr4 | 151053762 | 151053812 |
| chr4 | 151874069 | 151874119 |
| chr4 | 152707062 | 152707112 |
| chr4 | 152775191 | 152775241 |
| chr4 | 153514868 | 153514918 |
| chr4 | 153633265 | 153633315 |
| chr4 | 154051106 | 154051156 |
| chr4 | 154200689 | 154200739 |
| chr4 | 155171735 | 155171785 |
| chr4 | 156917967 | 156918017 |
| chr4 | 157092700 | 157092750 |
| chr11 | 67058650 | 67058700 |
| chr4 | 157443528 | 157443578 |
| chr4 | 157943725 | 157943775 |
| chr11 | 67099473 | 67099523 |
| chr4 | 158547252 | 158547302 |
| chr4 | 158549665 | 158549715 |
| chr4 | 158707921 | 158707971 |
| chr4 | 159649107 | 159649157 |
| chr4 | 160408961 | 160409011 |
| chr4 | 160517406 | 160517456 |
| chr10 | 13686047 | 13686097 |
| chr4 | 161424822 | 161424872 |
| chr4 | 161611356 | 161611406 |
| chr4 | 162013818 | 162013868 |
| chr4 | 162344051 | 162344101 |
| chr4 | 162392821 | 162392871 |
| chr10 | 13685834 | 13685884 |
| chr4 | 163603219 | 163603269 |
| chr4 | 163837193 | 163837243 |
| chr4 | 165049304 | 165049354 |
| chr4 | 165436681 | 165436731 |
| chr4 | 165436367 | 165436417 |
| chr4 | 165443068 | 165443118 |
| chr4 | 166297456 | 166297506 |
| chr4 | 166432660 | 166432710 |
| chr4 | 166432827 | 166432877 |
| chr4 | 166520004 | 166520054 |
| chr11 | 67366740 | 67366790 |
| chr4 | 166625111 | 166625161 |
| chr4 | 166625966 | 166626016 |
| chr4 | 167234601 | 167234651 |
| chr4 | 167473204 | 167473254 |
| chr4 | 167497710 | 167497760 |
| chr4 | 167874598 | 167874648 |
| chr4 | 168551215 | 168551265 |
| chr4 | 169073569 | 169073619 |
| chr4 | 169894449 | 169894499 |
| chr4 | 170639899 | 170639949 |
| chr4 | 170663504 | 170663554 |
| chr4 | 171493534 | 171493584 |
| chr4 | 175466338 | 175466388 |
| chr4 | 176025599 | 176025649 |
| chr4 | 176493588 | 176493638 |
| chr4 | 177026767 | 177026817 |
| chr4 | 177859031 | 177859081 |
| chr4 | 178430365 | 178430415 |
| chr4 | 178548636 | 178548686 |
| chr4 | 178549520 | 178549570 |
| chr4 | 179331312 | 179331362 |
| chr4 | 179345619 | 179345669 |
| chr4 | 179378967 | 179379017 |
| chr4 | 179654979 | 179655029 |
| chr4 | 179755826 | 179755876 |
| chr4 | 180225654 | 180225704 |
| chr4 | 182162455 | 182162505 |
| chr4 | 182162606 | 182162656 |
| chr4 | 182684615 | 182684665 |
| chr4 | 182755277 | 182755327 |
| chr4 | 182963851 | 182963901 |
| chr4 | 183187935 | 183187985 |
| chr4 | 184645832 | 184645882 |
| chr4 | 184681897 | 184681947 |
| chr4 | 184964214 | 184964264 |
| chr4 | 184964334 | 184964384 |
| chr4 | 184965109 | 184965159 |
| chr4 | 185184983 | 185185033 |
| chr4 | 185228517 | 185228567 |
| chr4 | 185229918 | 185229968 |
| chr4 | 185235567 | 185235617 |
| chr4 | 185235891 | 185235941 |
| chr4 | 185249851 | 185249901 |
| chr4 | 185250139 | 185250189 |
| chr4 | 185804164 | 185804214 |
| chr4 | 185942047 | 185942097 |
| chr4 | 185961090 | 185961140 |
| chr4 | 186124331 | 186124381 |
| chr4 | 186267811 | 186267861 |
| chr4 | 186620823 | 186620873 |
| chr4 | 186824022 | 186824072 |
| chr4 | 187675052 | 187675102 |
| chr4 | 187916651 | 187916701 |
| chr4 | 187976047 | 187976097 |
| chr4 | 188128051 | 188128101 |
| chr4 | 188155191 | 188155241 |
| chr4 | 188155491 | 188155541 |
| chr4 | 188155916 | 188155966 |
| chr4 | 188156277 | 188156327 |
| chr4 | 188156435 | 188156485 |
| chr4 | 188362485 | 188362535 |
| chr4 | 188362567 | 188362617 |
| chr4 | 188362796 | 188362846 |
| chr4 | 188363106 | 188363156 |
| chr4 | 188366924 | 188366974 |
| chr4 | 188512088 | 188512138 |
| chr4 | 188898341 | 188898391 |
| chr4 | 188984139 | 188984189 |
| chr4 | 189203432 | 189203482 |
| chr4 | 189443993 | 189444043 |
| chr4 | 189577663 | 189577713 |
| chr4 | 189693530 | 189693580 |
| chr4 | 189775456 | 189775506 |
| chr4 | 189776750 | 189776800 |
| chr4 | 189810150 | 189810200 |
| chr4 | 189884910 | 189884960 |
| chr4 | 189887480 | 189887530 |
| chr4 | 190013551 | 190013601 |
| chr4 | 190056762 | 190056812 |
| chr4 | 190277776 | 190277826 |
| chr4 | 190761968 | 190762018 |
| chr4 | 190906532 | 190906582 |
| chr4 | 190972775 | 190972825 |
| chr4 | 191170863 | 191170913 |
| chr4 | 191279911 | 191279961 |
| chr5 | 256482 | 256532 |
| chr5 | 399470 | 399520 |
| chr11 | 68563366 | 68563416 |
| chr11 | 68564771 | 68564821 |
| chr11 | 68571063 | 68571113 |
| chr11 | 68578384 | 68578434 |
| chr10 | 13816303 | 13816353 |
| chr5 | 1230802 | 1230852 |
| chr5 | 1231292 | 1231342 |
| chr10 | 13816183 | 13816233 |
| chr5 | 1369528 | 1369578 |
| chr5 | 1442955 | 1443005 |
| chr5 | 1580572 | 1580622 |
| chr5 | 1593694 | 1593744 |
| chr5 | 1650636 | 1650686 |
| chr5 | 1674289 | 1674339 |
| chr5 | 1676869 | 1676919 |
| chr5 | 1778206 | 1778256 |
| chr5 | 1860820 | 1860870 |
| chr5 | 2033420 | 2033470 |
| chr5 | 2208978 | 2209028 |
| chr5 | 2278185 | 2278235 |
| chr11 | 68972442 | 68972492 |
| chr5 | 3083945 | 3083995 |
| chr5 | 3682725 | 3682775 |
| chr5 | 6243629 | 6243679 |
| chr5 | 6578798 | 6578848 |
| chr5 | 6625391 | 6625441 |
| chr11 | 69119765 | 69119815 |
| chr11 | 69119657 | 69119707 |
| chr5 | 6820401 | 6820451 |
| chr5 | 6876057 | 6876107 |
| chr5 | 6875834 | 6875884 |
| chr5 | 6912016 | 6912066 |
| chr5 | 6930740 | 6930790 |
| chr5 | 6932896 | 6932946 |
| chr5 | 7090543 | 7090593 |
| chr5 | 7665555 | 7665605 |
| chr5 | 8012614 | 8012664 |
| chr5 | 8012488 | 8012538 |
| chr5 | 8502726 | 8502776 |
| chr5 | 8616246 | 8616296 |
| chr5 | 8807918 | 8807968 |
| chr11 | 69193964 | 69194014 |
| chr5 | 9551363 | 9551413 |
| chr5 | 10254793 | 10254843 |
| chr5 | 10254521 | 10254571 |
| chr5 | 10361782 | 10361832 |
| chr5 | 10910256 | 10910306 |
| chr5 | 12253733 | 12253783 |
| chr5 | 12392304 | 12392354 |
| chr5 | 12643194 | 12643244 |
| chr5 | 13469308 | 13469358 |
| chr5 | 13480101 | 13480151 |
| chr5 | 13942937 | 13942987 |
| chr5 | 14169093 | 14169143 |
| chr5 | 14966485 | 14966535 |
| chr11 | 69394399 | 69394449 |
| chr5 | 16489018 | 16489068 |
| chr5 | 16490266 | 16490316 |
| chr5 | 17175910 | 17175960 |
| chr5 | 17206260 | 17206310 |
| chr5 | 17989253 | 17989303 |
| chr5 | 18117585 | 18117635 |
| chr5 | 18156976 | 18157026 |
| chr5 | 18342099 | 18342149 |
| chr5 | 18399081 | 18399131 |
| chr5 | 18839149 | 18839199 |
| chr5 | 19473974 | 19474024 |
| chr5 | 20169105 | 20169155 |
| chr5 | 20892289 | 20892339 |
| chr5 | 21337684 | 21337734 |
| chr5 | 21573722 | 21573772 |
| chr5 | 22208423 | 22208473 |
| chr5 | 22684338 | 22684388 |
| chr5 | 22805974 | 22806024 |
| chr5 | 22924455 | 22924505 |
| chr5 | 23120757 | 23120807 |
| chr5 | 23135863 | 23135913 |
| chr5 | 23265007 | 23265057 |
| chr5 | 23271333 | 23271383 |
| chr5 | 23480741 | 23480791 |
| chr5 | 23732282 | 23732332 |
| chr5 | 24063439 | 24063489 |
| chr5 | 24094934 | 24094984 |
| chr5 | 25200234 | 25200284 |
| chr5 | 25325042 | 25325092 |
| chr5 | 25462370 | 25462420 |
| chr5 | 25938550 | 25938600 |
| chr5 | 26271413 | 26271463 |
| chr5 | 26513753 | 26513803 |
| chr5 | 26521626 | 26521676 |
| chr5 | 26747254 | 26747304 |
| chr5 | 26820059 | 26820109 |
| chr5 | 27109307 | 27109357 |
| chr5 | 27538832 | 27538882 |
| chr5 | 27625831 | 27625881 |
| chr5 | 27958909 | 27958959 |
| chr11 | 69718886 | 69718936 |
| chr11 | 69720359 | 69720409 |
| chr5 | 28896173 | 28896223 |
| chr5 | 29128043 | 29128093 |
| chr5 | 29219326 | 29219376 |
| chr5 | 29726832 | 29726882 |
| chr5 | 29831786 | 29831836 |
| chr5 | 29920260 | 29920310 |
| chr5 | 29994535 | 29994585 |
| chr5 | 30253349 | 30253399 |
| chr5 | 30475097 | 30475147 |
| chr5 | 30924089 | 30924139 |
| chr5 | 31716347 | 31716397 |
| chr11 | 69759599 | 69759649 |
| chr5 | 31848546 | 31848596 |
| chr5 | 32249406 | 32249456 |
| chr5 | 32363928 | 32363978 |
| chr5 | 32540283 | 32540333 |
| chr5 | 32607454 | 32607504 |
| chr5 | 33711979 | 33712029 |
| chr5 | 34366028 | 34366078 |
| chr5 | 35560324 | 35560374 |
| chr5 | 36079493 | 36079543 |
| chr5 | 36834733 | 36834783 |
| chr5 | 37173050 | 37173100 |
| chr5 | 37559014 | 37559064 |
| chr5 | 37558806 | 37558856 |
| chr5 | 38244379 | 38244429 |
| chr5 | 38816080 | 38816130 |
| chr5 | 38873987 | 38874037 |
| chr5 | 39144778 | 39144828 |
| chr5 | 39494380 | 39494430 |
| chr5 | 39712614 | 39712664 |
| chr5 | 40206692 | 40206742 |
| chr5 | 40303154 | 40303204 |
| chr5 | 40875544 | 40875594 |
| chr5 | 42967259 | 42967309 |
| chr5 | 43021322 | 43021372 |
| chr5 | 43196076 | 43196126 |
| chr5 | 43204176 | 43204226 |
| chr5 | 43295268 | 43295318 |
| chr5 | 44985597 | 44985647 |
| chr5 | 45884691 | 45884741 |
| chr5 | 49942088 | 49942138 |
| chr5 | 51829629 | 51829679 |
| chr5 | 52695082 | 52695132 |
| chr5 | 53762775 | 53762825 |
| chr5 | 53775371 | 53775421 |
| chr5 | 53784265 | 53784315 |
| chr5 | 53794012 | 53794062 |
| chr5 | 54022053 | 54022103 |
| chr11 | 70188073 | 70188123 |
| chr5 | 55620175 | 55620225 |
| chr5 | 55778009 | 55778059 |
| chr5 | 55777790 | 55777840 |
| chr5 | 56535353 | 56535403 |
| chr5 | 56887679 | 56887729 |
| chr5 | 57246987 | 57247037 |
| chr5 | 57823444 | 57823494 |
| chr5 | 57823224 | 57823274 |
| chr5 | 57940250 | 57940300 |
| chr5 | 58611402 | 58611452 |
| chr5 | 59711883 | 59711933 |
| chr5 | 61025428 | 61025478 |
| chr5 | 61379468 | 61379518 |
| chr5 | 61431233 | 61431283 |
| chr5 | 61512953 | 61513003 |
| chr5 | 64798423 | 64798473 |
| chr5 | 64848896 | 64848946 |
| chr5 | 65013719 | 65013769 |
| chr5 | 65180082 | 65180132 |
| chr5 | 67404186 | 67404236 |
| chr5 | 67490917 | 67490967 |
| chr5 | 68214152 | 68214202 |
| chr5 | 68214020 | 68214070 |
| chr5 | 68484850 | 68484900 |
| chr5 | 68557784 | 68557834 |
| chr5 | 68591236 | 68591286 |
| chr5 | 70775740 | 70775790 |
| chr5 | 71364334 | 71364384 |
| chr5 | 71818819 | 71818869 |
| chr5 | 73824224 | 73824274 |
| chr5 | 73996360 | 73996410 |
| chr5 | 74168616 | 74168666 |
| chr11 | 70609329 | 70609379 |
| chr5 | 74707816 | 74707866 |
| chr5 | 76115755 | 76115805 |
| chr5 | 76167067 | 76167117 |
| chr5 | 76360250 | 76360300 |
| chr5 | 76501677 | 76501727 |
| chr5 | 76757706 | 76757756 |
| chr5 | 76936282 | 76936332 |
| chr5 | 77934082 | 77934132 |
| chr5 | 77933993 | 77934043 |
| chr5 | 78611908 | 78611958 |
| chr11 | 70840290 | 70840340 |
| chr5 | 79722546 | 79722596 |
| chr5 | 79975562 | 79975612 |
| chr5 | 80373986 | 80374036 |
| chr5 | 80392512 | 80392562 |
| chr11 | 70912291 | 70912341 |
| chr10 | 14199990 | 14200040 |
| chr5 | 80967299 | 80967349 |
| chr5 | 81878264 | 81878314 |
| chr5 | 82103390 | 82103440 |
| chr5 | 82990458 | 82990508 |
| chr5 | 85421596 | 85421646 |
| chr5 | 85574344 | 85574394 |
| chr5 | 86381495 | 86381545 |
| chr5 | 86612830 | 86612880 |
| chr5 | 86612658 | 86612708 |
| chr5 | 87985272 | 87985322 |
| chr11 | 71074451 | 71074501 |
| chr5 | 88475927 | 88475977 |
| chr5 | 88697509 | 88697559 |
| chr5 | 88782968 | 88783018 |
| chr5 | 89693137 | 89693187 |
| chr5 | 89830809 | 89830859 |
| chr5 | 92471630 | 92471680 |
| chr5 | 95127471 | 95127521 |
| chr5 | 95127318 | 95127368 |
| chr5 | 96185921 | 96185971 |
| chr5 | 96185681 | 96185731 |
| chr5 | 97495687 | 97495737 |
| chr5 | 98816879 | 98816929 |
| chr5 | 99856900 | 99856950 |
| chr5 | 99992481 | 99992531 |
| chr5 | 99999729 | 99999779 |
| chr5 | 100131553 | 100131603 |
| chr5 | 100625253 | 100625303 |
| chr5 | 101174232 | 101174282 |
| chr5 | 101557060 | 101557110 |
| chr5 | 101847480 | 101847530 |
| chr5 | 104122435 | 104122485 |
| chr5 | 104323274 | 104323324 |
| chr5 | 104795983 | 104796033 |
| chr5 | 105649892 | 105649942 |
| chr5 | 105739154 | 105739204 |
| chr5 | 106157293 | 106157343 |
| chr5 | 106285518 | 106285568 |
| chr5 | 106287716 | 106287766 |
| chr5 | 106325045 | 106325095 |
| chr5 | 108114709 | 108114759 |
| chr5 | 108671146 | 108671196 |
| chr11 | 71695895 | 71695945 |
| chr5 | 111453449 | 111453499 |
| chr5 | 111643930 | 111643980 |
| chr5 | 112017852 | 112017902 |
| chr5 | 115136489 | 115136539 |
| chr5 | 115169987 | 115170037 |
| chr11 | 71886662 | 71886712 |
| chr11 | 71887819 | 71887869 |
| chr5 | 119505304 | 119505354 |
| chr5 | 121540580 | 121540630 |
| chr5 | 121895650 | 121895700 |
| chr5 | 122382726 | 122382776 |
| chr5 | 123279147 | 123279197 |
| chr5 | 123297030 | 123297080 |
| chr5 | 123465068 | 123465118 |
| chr5 | 123464870 | 123464920 |
| chr11 | 72101520 | 72101570 |
| chr5 | 124369966 | 124370016 |
| chr5 | 124463318 | 124463368 |
| chr5 | 125209670 | 125209720 |
| chr5 | 126087963 | 126088013 |
| chr5 | 126168217 | 126168267 |
| chr5 | 126534916 | 126534966 |
| chr5 | 130301407 | 130301457 |
| chr5 | 130647790 | 130647840 |
| chr5 | 131225263 | 131225313 |
| chr5 | 131229208 | 131229258 |
| chr5 | 131461571 | 131461621 |
| chr5 | 131697694 | 131697744 |
| chr5 | 131701344 | 131701394 |
| chr5 | 131772240 | 131772290 |
| chr5 | 132080473 | 132080523 |
| chr5 | 132454789 | 132454839 |
| chr5 | 133029230 | 133029280 |
| chr11 | 72688134 | 72688184 |
| chr5 | 134256938 | 134256988 |
| chr5 | 135361042 | 135361092 |
| chr5 | 138289249 | 138289299 |
| chr5 | 138621902 | 138621952 |
| chr5 | 139632166 | 139632216 |
| chr5 | 140695468 | 140695518 |
| chr5 | 141434767 | 141434817 |
| chr5 | 141669062 | 141669112 |
| chr5 | 142140523 | 142140573 |
| chr11 | 73395269 | 73395319 |
| chr5 | 143695596 | 143695646 |
| chr5 | 144065281 | 144065331 |
| chr5 | 144553386 | 144553436 |
| chr5 | 145264606 | 145264656 |
| chr5 | 148350176 | 148350226 |
| chr11 | 73890132 | 73890182 |
| chr11 | 73979736 | 73979786 |
| chr5 | 151203034 | 151203084 |
| chr5 | 151255899 | 151255949 |
| chr5 | 152031779 | 152031829 |
| chr5 | 153565323 | 153565373 |
| chr5 | 153587544 | 153587594 |
| chr5 | 157889214 | 157889264 |
| chr5 | 159621642 | 159621692 |
| chr5 | 159621539 | 159621589 |
| chr5 | 160346629 | 160346679 |
| chr5 | 160346343 | 160346393 |
| chr5 | 160403079 | 160403129 |
| chr5 | 160806418 | 160806468 |
| chr5 | 161114538 | 161114588 |
| chr5 | 162164796 | 162164846 |
| chr5 | 162323487 | 162323537 |
| chr5 | 162509915 | 162509965 |
| chr10 | 14701666 | 14701716 |
| chr5 | 162902729 | 162902779 |
| chr11 | 74794734 | 74794784 |
| chr5 | 164302417 | 164302467 |
| chr5 | 164469430 | 164469480 |
| chr10 | 14745869 | 14745919 |
| chr5 | 166142781 | 166142831 |
| chr11 | 74851651 | 74851701 |
| chr5 | 168028580 | 168028630 |
| chr5 | 168546595 | 168546645 |
| chr11 | 75018076 | 75018126 |
| chr5 | 169712656 | 169712706 |
| chr5 | 169756391 | 169756441 |
| chr5 | 170150488 | 170150538 |
| chr11 | 75151841 | 75151891 |
| chr5 | 170916339 | 170916389 |
| chr5 | 171600066 | 171600116 |
| chr5 | 171935093 | 171935143 |
| chr5 | 171963844 | 171963894 |
| chr5 | 171975107 | 171975157 |
| chr5 | 172317680 | 172317730 |
| chr5 | 172586454 | 172586504 |
| chr11 | 75478237 | 75478287 |
| chr5 | 172899110 | 172899160 |
| chr5 | 172981585 | 172981635 |
| chr5 | 173416440 | 173416490 |
| chr5 | 173498268 | 173498318 |
| chr5 | 173740399 | 173740449 |
| chr5 | 173936851 | 173936901 |
| chr5 | 174408382 | 174408432 |
| chr5 | 174613900 | 174613950 |
| chr5 | 174965181 | 174965231 |
| chr5 | 175891683 | 175891733 |
| chr5 | 175967923 | 175967973 |
| chr5 | 176309792 | 176309842 |
| chr5 | 176388024 | 176388074 |
| chr5 | 178017199 | 178017249 |
| chr5 | 178087909 | 178087959 |
| chr5 | 178087774 | 178087824 |
| chr11 | 76173418 | 76173468 |
| chr5 | 178511855 | 178511905 |
| chr5 | 178664321 | 178664371 |
| chr11 | 76312285 | 76312335 |
| chr5 | 179045018 | 179045068 |
| chr5 | 179758671 | 179758721 |
| chr5 | 179789915 | 179789965 |
| chr5 | 179840095 | 179840145 |
| chr5 | 180527226 | 180527276 |
| chr5 | 180527090 | 180527140 |
| chr5 | 180659178 | 180659228 |
| chr6 | 794680 | 794730 |
| chr6 | 883886 | 883936 |
| chr6 | 2407912 | 2407962 |
| chr6 | 2748663 | 2748713 |
| chr6 | 2750521 | 2750571 |
| chr6 | 3138909 | 3138959 |
| chr6 | 3198794 | 3198844 |
| chr6 | 3273677 | 3273727 |
| chr11 | 77084915 | 77084965 |
| chr6 | 3845039 | 3845089 |
| chr6 | 3876977 | 3877027 |
| chr6 | 4111887 | 4111937 |
| chr6 | 4185691 | 4185741 |
| chr11 | 77275148 | 77275198 |
| chr6 | 6718656 | 6718706 |
| chr6 | 6779921 | 6779971 |
| chr6 | 6938846 | 6938896 |
| chr6 | 7266313 | 7266363 |
| chr6 | 7267792 | 7267842 |
| chr11 | 77472810 | 77472860 |
| chr6 | 7906796 | 7906846 |
| chr6 | 9523798 | 9523848 |
| chr6 | 9599046 | 9599096 |
| chr6 | 9844437 | 9844487 |
| chr11 | 77590133 | 77590183 |
| chr6 | 10101594 | 10101644 |
| chr6 | 10244944 | 10244994 |
| chr6 | 10574982 | 10575032 |
| chr6 | 10686949 | 10686999 |
| chr6 | 10966242 | 10966292 |
| chr6 | 11426770 | 11426820 |
| chr6 | 11426577 | 11426627 |
| chr6 | 11606829 | 11606879 |
| chr6 | 11889422 | 11889472 |
| chr6 | 12025216 | 12025266 |
| chr6 | 13954745 | 13954795 |
| chr6 | 14124736 | 14124786 |
| chr11 | 78047790 | 78047840 |
| chr6 | 14537808 | 14537858 |
| chr6 | 14848793 | 14848843 |
| chr6 | 15647977 | 15648027 |
| chr11 | 78349472 | 78349522 |
| chr6 | 16916013 | 16916063 |
| chr6 | 17221147 | 17221197 |
| chr6 | 17374889 | 17374939 |
| chr6 | 17450880 | 17450930 |
| chr6 | 18118162 | 18118212 |
| chr11 | 79107121 | 79107171 |
| chr6 | 18424258 | 18424308 |
| chr6 | 18435214 | 18435264 |
| chr6 | 19785228 | 19785278 |
| chr6 | 19892444 | 19892494 |
| chr11 | 79577086 | 79577136 |
| chr6 | 21096792 | 21096842 |
| chr11 | 80225328 | 80225378 |
| chr11 | 80242000 | 80242050 |
| chr6 | 21466481 | 21466531 |
| chr6 | 21762273 | 21762323 |
| chr6 | 21854982 | 21855032 |
| chr6 | 22463435 | 22463485 |
| chr6 | 23047725 | 23047775 |
| chr6 | 23912217 | 23912267 |
| chr6 | 24142989 | 24143039 |
| chr6 | 25026186 | 25026236 |
| chr6 | 26253667 | 26253717 |
| chr11 | 81618370 | 81618420 |
| chr6 | 26483780 | 26483830 |
| chr6 | 26552691 | 26552741 |
| chr6 | 26552629 | 26552679 |
| chr6 | 27284369 | 27284419 |
| chr6 | 27664458 | 27664508 |
| chr6 | 27690124 | 27690174 |
| chr6 | 27739211 | 27739261 |
| chr6 | 27815049 | 27815099 |
| chr6 | 28077381 | 28077431 |
| chr6 | 28298526 | 28298576 |
| chr6 | 28419568 | 28419618 |
| chr6 | 28432162 | 28432212 |
| chr6 | 28431869 | 28431919 |
| chr6 | 28560635 | 28560685 |
| chr6 | 28629866 | 28629916 |
| chr6 | 28705039 | 28705089 |
| chr6 | 28945433 | 28945483 |
| chr6 | 28946011 | 28946061 |
| chr6 | 29680115 | 29680165 |
| chr6 | 29759374 | 29759424 |
| chr6 | 29953086 | 29953136 |
| chr6 | 29952883 | 29952933 |
| chr6 | 30007332 | 30007382 |
| chr6 | 30293039 | 30293089 |
| chr6 | 30766395 | 30766445 |
| chr6 | 31075637 | 31075687 |
| chr6 | 31112295 | 31112345 |
| chr6 | 31160130 | 31160180 |
| chr6 | 31535421 | 31535471 |
| chr6 | 31535277 | 31535327 |
| chr11 | 83181720 | 83181770 |
| chr11 | 83181457 | 83181507 |
| chr6 | 32163008 | 32163058 |
| chr6 | 32668245 | 32668295 |
| chr6 | 33509383 | 33509433 |
| chr6 | 33695868 | 33695918 |
| chr6 | 33744266 | 33744316 |
| chr6 | 33858828 | 33858878 |
| chr6 | 33882376 | 33882426 |
| chr6 | 34506113 | 34506163 |
| chr6 | 34768366 | 34768416 |
| chr6 | 34854866 | 34854916 |
| chr6 | 35552087 | 35552137 |
| chr6 | 35553552 | 35553602 |
| chr6 | 35571925 | 35571975 |
| chr6 | 35939722 | 35939772 |
| chr6 | 36564449 | 36564499 |
| chr6 | 36649234 | 36649284 |
| chr6 | 36878323 | 36878373 |
| chr6 | 36878502 | 36878552 |
| chr6 | 37115469 | 37115519 |
| chr6 | 37260285 | 37260335 |
| chr6 | 37367387 | 37367437 |
| chr6 | 37585562 | 37585612 |
| chr6 | 37591343 | 37591393 |
| chr6 | 37610136 | 37610186 |
| chr6 | 37798810 | 37798860 |
| chr6 | 38123665 | 38123715 |
| chr6 | 38811313 | 38811363 |
| chr6 | 39342917 | 39342967 |
| chr6 | 39591164 | 39591214 |
| chr6 | 39833183 | 39833233 |
| chr6 | 40300972 | 40301022 |
| chr6 | 40455810 | 40455860 |
| chr6 | 40530566 | 40530616 |
| chr6 | 41038844 | 41038894 |
| chr6 | 42183215 | 42183265 |
| chr6 | 42495594 | 42495644 |
| chr6 | 42654824 | 42654874 |
| chr6 | 43395402 | 43395452 |
| chr6 | 43397592 | 43397642 |
| chr6 | 43399112 | 43399162 |
| chr6 | 44838237 | 44838287 |
| chr6 | 47168967 | 47169017 |
| chr6 | 50961577 | 50961627 |
| chr10 | 15290849 | 15290899 |
| chr6 | 52404923 | 52404973 |
| chr6 | 52404817 | 52404867 |
| chr6 | 53062207 | 53062257 |
| chr6 | 53135285 | 53135335 |
| chr11 | 87854950 | 87855000 |
| chr11 | 88029680 | 88029730 |
| chr6 | 55878078 | 55878128 |
| chr6 | 56796640 | 56796690 |
| chr6 | 57681279 | 57681329 |
| chr6 | 62334162 | 62334212 |
| chr11 | 88705798 | 88705848 |
| chr6 | 63980683 | 63980733 |
| chr6 | 63981941 | 63981991 |
| chr6 | 64126967 | 64127017 |
| chr6 | 64291721 | 64291771 |
| chr6 | 64832193 | 64832243 |
| chr6 | 66232320 | 66232370 |
| chr11 | 89124528 | 89124578 |
| chr6 | 66940377 | 66940427 |
| chr11 | 89172265 | 89172315 |
| chr6 | 68839330 | 68839380 |
| chr11 | 89544591 | 89544641 |
| chr6 | 69281565 | 69281615 |
| chr6 | 69408524 | 69408574 |
| chr6 | 70306607 | 70306657 |
| chr6 | 71271736 | 71271786 |
| chr6 | 71296452 | 71296502 |
| chr11 | 89772110 | 89772160 |
| chr6 | 72895922 | 72895972 |
| chr10 | 15347118 | 15347168 |
| chr11 | 90082072 | 90082122 |
| chr6 | 74726824 | 74726874 |
| chr11 | 90428685 | 90428735 |
| chr10 | 15349974 | 15350024 |
| chr11 | 90689855 | 90689905 |
| chr6 | 76257273 | 76257323 |
| chr6 | 77391973 | 77392023 |
| chr6 | 77580848 | 77580898 |
| chr6 | 77582412 | 77582462 |
| chr6 | 78646575 | 78646625 |
| chr6 | 78805948 | 78805998 |
| chr6 | 79604309 | 79604359 |
| chr6 | 79604240 | 79604290 |
| chr6 | 80018768 | 80018818 |
| chr6 | 80018515 | 80018565 |
| chr11 | 91343026 | 91343076 |
| chr6 | 80609400 | 80609450 |
| chr6 | 80613912 | 80613962 |
| chr6 | 80963036 | 80963086 |
| chr6 | 81701502 | 81701552 |
| chr6 | 83084521 | 83084571 |
| chr6 | 83293368 | 83293418 |
| chr11 | 91901264 | 91901314 |
| chr6 | 85499852 | 85499902 |
| chr6 | 86622292 | 86622342 |
| chr6 | 86648964 | 86649014 |
| chr6 | 86911446 | 86911496 |
| chr6 | 87685770 | 87685820 |
| chr6 | 87763558 | 87763608 |
| chr6 | 88717307 | 88717357 |
| chr6 | 88978305 | 88978355 |
| chr6 | 89945771 | 89945821 |
| chr6 | 90250211 | 90250261 |
| chr11 | 92627531 | 92627581 |
| chr6 | 91979169 | 91979219 |
| chr11 | 92857514 | 92857564 |
| chr6 | 94547928 | 94547978 |
| chr6 | 94803351 | 94803401 |
| chr6 | 95556345 | 95556395 |
| chr6 | 95628047 | 95628097 |
| chr6 | 96870710 | 96870760 |
| chr6 | 96887954 | 96888004 |
| chr6 | 97230086 | 97230136 |
| chr6 | 97513025 | 97513075 |
| chr11 | 93343104 | 93343154 |
| chr6 | 103252161 | 103252211 |
| chr6 | 104093204 | 104093254 |
| chr6 | 104917906 | 104917956 |
| chr6 | 105214037 | 105214087 |
| chr6 | 105700076 | 105700126 |
| chr6 | 105710211 | 105710261 |
| chr6 | 105747608 | 105747658 |
| chr10 | 15438107 | 15438157 |
| chr6 | 107177781 | 107177831 |
| chr6 | 109199198 | 109199248 |
| chr11 | 94209244 | 94209294 |
| chr6 | 109730091 | 109730141 |
| chr6 | 110450587 | 110450637 |
| chr6 | 111209537 | 111209587 |
| chr6 | 111214329 | 111214379 |
| chr6 | 112511635 | 112511685 |
| chr6 | 112945363 | 112945413 |
| chr6 | 114341316 | 114341366 |
| chr6 | 114616890 | 114616940 |
| chr6 | 115011254 | 115011304 |
| chr6 | 115232969 | 115233019 |
| chr6 | 116131504 | 116131554 |
| chr6 | 116186093 | 116186143 |
| chr6 | 119153102 | 119153152 |
| chr6 | 119162040 | 119162090 |
| chr6 | 120106987 | 120107037 |
| chr6 | 121500132 | 121500182 |
| chr6 | 121835924 | 121835974 |
| chr6 | 122458270 | 122458320 |
| chr6 | 122848966 | 122849016 |
| chr6 | 122896591 | 122896641 |
| chr6 | 123867632 | 123867682 |
| chr6 | 124264382 | 124264432 |
| chr6 | 124874258 | 124874308 |
| chr6 | 127390271 | 127390321 |
| chr6 | 127541536 | 127541586 |
| chr6 | 127725475 | 127725525 |
| chr6 | 129613123 | 129613173 |
| chr11 | 96400830 | 96400880 |
| chr6 | 131069295 | 131069345 |
| chr6 | 131861203 | 131861253 |
| chr6 | 132473650 | 132473700 |
| chr6 | 132473418 | 132473468 |
| chr6 | 132906121 | 132906171 |
| chr6 | 133250655 | 133250705 |
| chr6 | 133665465 | 133665515 |
| chr11 | 97183407 | 97183457 |
| chr6 | 134369035 | 134369085 |
| chr6 | 134838039 | 134838089 |
| chr11 | 97450060 | 97450110 |
| chr6 | 135174097 | 135174147 |
| chr6 | 135380018 | 135380068 |
| chr11 | 97508624 | 97508674 |
| chr6 | 135946996 | 135947046 |
| chr6 | 137411784 | 137411834 |
| chr11 | 97770236 | 97770286 |
| chr6 | 139036981 | 139037031 |
| chr6 | 139039407 | 139039457 |
| chr6 | 139299771 | 139299821 |
| chr6 | 139299629 | 139299679 |
| chr6 | 140194195 | 140194245 |
| chr6 | 140543421 | 140543471 |
| chr11 | 98375854 | 98375904 |
| chr11 | 98499635 | 98499685 |
| chr6 | 144523463 | 144523513 |
| chr6 | 145360709 | 145360759 |
| chr6 | 146569176 | 146569226 |
| chr6 | 147401728 | 147401778 |
| chr6 | 147835252 | 147835302 |
| chr6 | 148129775 | 148129825 |
| chr6 | 149092317 | 149092367 |
| chr11 | 99456629 | 99456679 |
| chr6 | 150407323 | 150407373 |
| chr6 | 150685581 | 150685631 |
| chr6 | 151201244 | 151201294 |
| chr6 | 151536131 | 151536181 |
| chr6 | 152078240 | 152078290 |
| chr6 | 153016984 | 153017034 |
| chr6 | 153129255 | 153129305 |
| chr6 | 153503258 | 153503308 |
| chr6 | 153550052 | 153550102 |
| chr6 | 154464358 | 154464408 |
| chr6 | 154503982 | 154504032 |
| chr6 | 154870278 | 154870328 |
| chr6 | 154967986 | 154968036 |
| chr11 | 100636870 | 100636920 |
| chr11 | 100680784 | 100680834 |
| chr6 | 155678569 | 155678619 |
| chr11 | 100839799 | 100839849 |
| chr6 | 156435432 | 156435482 |
| chr6 | 156712221 | 156712271 |
| chr6 | 157001924 | 157001974 |
| chr6 | 157048197 | 157048247 |
| chr6 | 157230213 | 157230263 |
| chr11 | 101128220 | 101128270 |
| chr11 | 101258719 | 101258769 |
| chr6 | 157699280 | 157699330 |
| chr6 | 158028345 | 158028395 |
| chr11 | 101423018 | 101423068 |
| chr6 | 158814989 | 158815039 |
| chr6 | 159399357 | 159399407 |
| chr6 | 159546677 | 159546727 |
| chr6 | 160167226 | 160167276 |
| chr6 | 160421114 | 160421164 |
| chr11 | 101864842 | 101864892 |
| chr11 | 101904832 | 101904882 |
| chr6 | 161205663 | 161205713 |
| chr6 | 161686209 | 161686259 |
| chr6 | 161686398 | 161686448 |
| chr6 | 161686574 | 161686624 |
| chr6 | 161760821 | 161760871 |
| chr6 | 162308152 | 162308202 |
| chr6 | 163224049 | 163224099 |
| chr6 | 163388692 | 163388742 |
| chr6 | 164018015 | 164018065 |
| chr6 | 164409512 | 164409562 |
| chr6 | 165149529 | 165149579 |
| chr6 | 165574691 | 165574741 |
| chr6 | 165620451 | 165620501 |
| chr11 | 102791860 | 102791910 |
| chr6 | 166014491 | 166014541 |
| chr11 | 102935242 | 102935292 |
| chr11 | 102945098 | 102945148 |
| chr11 | 103302919 | 103302969 |
| chr6 | 166595161 | 166595211 |
| chr11 | 103636850 | 103636900 |
| chr6 | 166853530 | 166853580 |
| chr6 | 166881545 | 166881595 |
| chr6 | 166881337 | 166881387 |
| chr6 | 167077091 | 167077141 |
| chr6 | 167233264 | 167233314 |
| chr6 | 167387267 | 167387317 |
| chr6 | 167422645 | 167422695 |
| chr6 | 167469630 | 167469680 |
| chr6 | 167469713 | 167469763 |
| chr6 | 167469950 | 167470000 |
| chr6 | 167470298 | 167470348 |
| chr11 | 104666946 | 104666996 |
| chr6 | 167902362 | 167902412 |
| chr6 | 167964844 | 167964894 |
| chr6 | 168154279 | 168154329 |
| chr11 | 104886441 | 104886491 |
| chr6 | 168228942 | 168228992 |
| chr11 | 105704033 | 105704083 |
| chr6 | 168842830 | 168842880 |
| chr10 | 16416135 | 16416185 |
| chr6 | 169655913 | 169655963 |
| chr6 | 169731254 | 169731304 |
| chr6 | 169941359 | 169941409 |
| chr6 | 170021365 | 170021415 |
| chr6 | 170270387 | 170270437 |
| chr6 | 170270587 | 170270637 |
| chr6 | 170270844 | 170270894 |
| chr6 | 170352917 | 170352967 |
| chr6 | 170646790 | 170646840 |
| chr6 | 170650606 | 170650656 |
| chr7 | 213514 | 213564 |
| chr7 | 214270 | 214320 |
| chr7 | 249015 | 249065 |
| chr7 | 652304 | 652354 |
| chr7 | 883665 | 883715 |
| chr7 | 959254 | 959304 |
| chr7 | 963304 | 963354 |
| chr7 | 976211 | 976261 |
| chr7 | 1033185 | 1033235 |
| chr7 | 1095408 | 1095458 |
| chr7 | 1113462 | 1113512 |
| chr11 | 107363789 | 107363839 |
| chr11 | 107384696 | 107384746 |
| chr7 | 1304506 | 1304556 |
| chr7 | 1543684 | 1543734 |
| chr7 | 1859001 | 1859051 |
| chr7 | 2202097 | 2202147 |
| chr7 | 2381091 | 2381141 |
| chr7 | 2599983 | 2600033 |
| chr7 | 2609492 | 2609542 |
| chr7 | 2644733 | 2644783 |
| chr7 | 2839014 | 2839064 |
| chr7 | 2849904 | 2849954 |
| chr7 | 2953233 | 2953283 |
| chr7 | 3368976 | 3369026 |
| chr7 | 3626885 | 3626935 |
| chr7 | 3791401 | 3791451 |
| chr7 | 3976774 | 3976824 |
| chr7 | 4239032 | 4239082 |
| chr7 | 4313012 | 4313062 |
| chr7 | 4437466 | 4437516 |
| chr11 | 108760717 | 108760767 |
| chr7 | 4752613 | 4752663 |
| chr7 | 5882307 | 5882357 |
| chr7 | 6495299 | 6495349 |
| chr7 | 8619853 | 8619903 |
| chr7 | 9078817 | 9078867 |
| chr7 | 9106248 | 9106298 |
| chr7 | 9441381 | 9441431 |
| chr7 | 10645580 | 10645630 |
| chr11 | 110297106 | 110297156 |
| chr7 | 12517848 | 12517898 |
| chr7 | 13233606 | 13233656 |
| chr7 | 13287603 | 13287653 |
| chr7 | 13467906 | 13467956 |
| chr7 | 14584196 | 14584246 |
| chr7 | 14861665 | 14861715 |
| chr7 | 15486372 | 15486422 |
| chr7 | 15826907 | 15826957 |
| chr7 | 16314666 | 16314716 |
| chr7 | 16314370 | 16314420 |
| chr7 | 19029198 | 19029248 |
| chr7 | 19030908 | 19030958 |
| chr7 | 19223028 | 19223078 |
| chr7 | 19828935 | 19828985 |
| chr7 | 21880835 | 21880885 |
| chr7 | 22117888 | 22117938 |
| chr7 | 22234162 | 22234212 |
| chr7 | 22337786 | 22337836 |
| chr7 | 22718495 | 22718545 |
| chr7 | 22718365 | 22718415 |
| chr7 | 22895240 | 22895290 |
| chr7 | 22993380 | 22993430 |
| chr7 | 23196919 | 23196969 |
| chr7 | 24031433 | 24031483 |
| chr7 | 24948162 | 24948212 |
| chr7 | 26654295 | 26654345 |
| chr7 | 27558058 | 27558108 |
| chr7 | 27557856 | 27557906 |
| chr7 | 28014400 | 28014450 |
| chr7 | 28248686 | 28248736 |
| chr7 | 28248597 | 28248647 |
| chr7 | 29036122 | 29036172 |
| chr7 | 31410152 | 31410202 |
| chr7 | 32225021 | 32225071 |
| chr7 | 32532409 | 32532459 |
| chr7 | 32703958 | 32704008 |
| chr7 | 32922137 | 32922187 |
| chr7 | 32985831 | 32985881 |
| chr7 | 33384261 | 33384311 |
| chr7 | 37398535 | 37398585 |
| chr7 | 37530437 | 37530487 |
| chr7 | 37530357 | 37530407 |
| chr7 | 38859324 | 38859374 |
| chr7 | 39341824 | 39341874 |
| chr7 | 39696832 | 39696882 |
| chr7 | 39940527 | 39940577 |
| chr7 | 40987697 | 40987747 |
| chr7 | 42251464 | 42251514 |
| chr7 | 43761948 | 43761998 |
| chr11 | 113081706 | 113081756 |
| chr7 | 44124190 | 44124240 |
| chr7 | 44560407 | 44560457 |
| chr11 | 113174339 | 113174389 |
| chr7 | 44637395 | 44637445 |
| chr7 | 44832493 | 44832543 |
| chr7 | 44999757 | 44999807 |
| chr7 | 45034462 | 45034512 |
| chr7 | 45422489 | 45422539 |
| chr7 | 45860214 | 45860264 |
| chr11 | 113272824 | 113272874 |
| chr7 | 46409458 | 46409508 |
| chr7 | 46434126 | 46434176 |
| chr7 | 46988357 | 46988407 |
| chr7 | 47366209 | 47366259 |
| chr7 | 47457236 | 47457286 |
| chr7 | 48165932 | 48165982 |
| chr7 | 48165683 | 48165733 |
| chr11 | 113424814 | 113424864 |
| chr7 | 51145436 | 51145486 |
| chr7 | 52654800 | 52654850 |
| chr7 | 52684086 | 52684136 |
| chr7 | 53621070 | 53621120 |
| chr7 | 53621388 | 53621438 |
| chr7 | 55049525 | 55049575 |
| chr7 | 55217291 | 55217341 |
| chr7 | 55701804 | 55701854 |
| chr7 | 55814852 | 55814902 |
| chr7 | 57398329 | 57398379 |
| chr7 | 62058117 | 62058167 |
| chr7 | 64486710 | 64486760 |
| chr7 | 64620694 | 64620744 |
| chr7 | 64859326 | 64859376 |
| chr7 | 64859931 | 64859981 |
| chr11 | 114462889 | 114462939 |
| chr11 | 114543399 | 114543449 |
| chr7 | 65197598 | 65197648 |
| chr7 | 66787603 | 66787653 |
| chr7 | 66848441 | 66848491 |
| chr7 | 67005021 | 67005071 |
| chr7 | 67230852 | 67230902 |
| chr7 | 67397640 | 67397690 |
| chr7 | 67433571 | 67433621 |
| chr7 | 67474895 | 67474945 |
| chr7 | 67621994 | 67622044 |
| chr7 | 68368621 | 68368671 |
| chr7 | 69839805 | 69839855 |
| chr7 | 69914072 | 69914122 |
| chr7 | 69916233 | 69916283 |
| chr7 | 70023179 | 70023229 |
| chr7 | 70263783 | 70263833 |
| chr7 | 70761298 | 70761348 |
| chr7 | 70808682 | 70808732 |
| chr7 | 70855674 | 70855724 |
| chr7 | 71803805 | 71803855 |
| chr7 | 72389002 | 72389052 |
| chr7 | 72907706 | 72907756 |
| chr7 | 73536445 | 73536495 |
| chr7 | 73697381 | 73697431 |
| chr7 | 73903301 | 73903351 |
| chr7 | 74987338 | 74987388 |
| chr7 | 75006706 | 75006756 |
| chr7 | 75195014 | 75195064 |
| chr7 | 75376621 | 75376671 |
| chr7 | 75438553 | 75438603 |
| chr7 | 76984134 | 76984184 |
| chr11 | 116229932 | 116229982 |
| chr7 | 77317121 | 77317171 |
| chr7 | 79610715 | 79610765 |
| chr7 | 80394946 | 80394996 |
| chr7 | 80727622 | 80727672 |
| chr7 | 81391358 | 81391408 |
| chr7 | 81443708 | 81443758 |
| chr7 | 81755502 | 81755552 |
| chr7 | 83176543 | 83176593 |
| chr7 | 83363816 | 83363866 |
| chr7 | 84117849 | 84117899 |
| chr7 | 84117906 | 84117956 |
| chr7 | 84171107 | 84171157 |
| chr7 | 85469154 | 85469204 |
| chr7 | 89187104 | 89187154 |
| chr7 | 90775449 | 90775499 |
| chr7 | 90830875 | 90830925 |
| chr7 | 91719568 | 91719618 |
| chr7 | 92643844 | 92643894 |
| chr7 | 93027937 | 93027987 |
| chr7 | 94961541 | 94961591 |
| chr7 | 95344897 | 95344947 |
| chr7 | 97275229 | 97275279 |
| chr7 | 97281936 | 97281986 |
| chr7 | 97753959 | 97754009 |
| chr7 | 97909018 | 97909068 |
| chr7 | 97946138 | 97946188 |
| chr7 | 98435078 | 98435128 |
| chr7 | 98528803 | 98528853 |
| chr7 | 98698387 | 98698437 |
| chr7 | 99087232 | 99087282 |
| chr7 | 99150210 | 99150260 |
| chr7 | 99188629 | 99188679 |
| chr7 | 99276102 | 99276152 |
| chr7 | 99489080 | 99489130 |
| chr7 | 99563682 | 99563732 |
| chr7 | 99741950 | 99742000 |
| chr7 | 100124897 | 100124947 |
| chr7 | 100126203 | 100126253 |
| chr7 | 100127855 | 100127905 |
| chr11 | 117107796 | 117107846 |
| chr7 | 100494307 | 100494357 |
| chr10 | 17318769 | 17318819 |
| chr7 | 100544062 | 100544112 |
| chr7 | 100787374 | 100787424 |
| chr7 | 100993964 | 100994014 |
| chr7 | 102362459 | 102362509 |
| chr7 | 104622716 | 104622766 |
| chr7 | 106191516 | 106191566 |
| chr7 | 106529957 | 106530007 |
| chr7 | 107081893 | 107081943 |
| chr7 | 107222371 | 107222421 |
| chr7 | 107282126 | 107282176 |
| chr7 | 107282992 | 107283042 |
| chr7 | 107388612 | 107388662 |
| chr7 | 107594686 | 107594736 |
| chr7 | 109931413 | 109931463 |
| chr7 | 110266516 | 110266566 |
| chr7 | 110694806 | 110694856 |
| chr7 | 111992638 | 111992688 |
| chr7 | 112338636 | 112338686 |
| chr7 | 112986720 | 112986770 |
| chr7 | 115481261 | 115481311 |
| chr7 | 116070953 | 116071003 |
| chr7 | 116598271 | 116598321 |
| chr7 | 117898640 | 117898690 |
| chr7 | 118383067 | 118383117 |
| chr7 | 118884070 | 118884120 |
| chr7 | 119425125 | 119425175 |
| chr7 | 120844869 | 120844919 |
| chr7 | 122123683 | 122123733 |
| chr7 | 122154571 | 122154621 |
| chr7 | 122229530 | 122229580 |
| chr7 | 122231003 | 122231053 |
| chr7 | 122645761 | 122645811 |
| chr7 | 122711774 | 122711824 |
| chr7 | 122760060 | 122760110 |
| chr7 | 124764375 | 124764425 |
| chr10 | 17432944 | 17432994 |
| chr7 | 125026739 | 125026789 |
| chr7 | 125028182 | 125028232 |
| chr7 | 125240648 | 125240698 |
| chr7 | 125278747 | 125278797 |
| chr11 | 118116141 | 118116191 |
| chr7 | 126106333 | 126106383 |
| chr11 | 118151285 | 118151335 |
| chr10 | 17433699 | 17433749 |
| chr7 | 127302549 | 127302599 |
| chr11 | 118181558 | 118181608 |
| chr7 | 127882691 | 127882741 |
| chr11 | 118256647 | 118256697 |
| chr7 | 129162287 | 129162337 |
| chr10 | 17466616 | 17466666 |
| chr7 | 130617133 | 130617183 |
| chr7 | 130618353 | 130618403 |
| chr7 | 130619756 | 130619806 |
| chr11 | 118353449 | 118353499 |
| chr7 | 130715523 | 130715573 |
| chr7 | 130741139 | 130741189 |
| chr7 | 130765665 | 130765715 |
| chr7 | 131615325 | 131615375 |
| chr7 | 132000178 | 132000228 |
| chr7 | 132372182 | 132372232 |
| chr7 | 133434949 | 133434999 |
| chr7 | 133619782 | 133619832 |
| chr7 | 134033961 | 134034011 |
| chr7 | 134033892 | 134033942 |
| chr7 | 134360358 | 134360408 |
| chr7 | 134627117 | 134627167 |
| chr7 | 134818844 | 134818894 |
| chr7 | 135292474 | 135292524 |
| chr7 | 136415656 | 136415706 |
| chr7 | 136725685 | 136725735 |
| chr7 | 136734028 | 136734078 |
| chr7 | 137228260 | 137228310 |
| chr7 | 137296347 | 137296397 |
| chr7 | 138020637 | 138020687 |
| chr7 | 138618141 | 138618191 |
| chr7 | 139082577 | 139082627 |
| chr7 | 139082274 | 139082324 |
| chr7 | 139399572 | 139399622 |
| chr7 | 141357856 | 141357906 |
| chr7 | 141687947 | 141687997 |
| chr7 | 142289936 | 142289986 |
| chr7 | 144027389 | 144027439 |
| chr7 | 144515698 | 144515748 |
| chr7 | 144568440 | 144568490 |
| chr7 | 144603266 | 144603316 |
| chr7 | 145558436 | 145558486 |
| chr7 | 146323510 | 146323560 |
| chr7 | 146878109 | 146878159 |
| chr7 | 147540919 | 147540969 |
| chr7 | 147548429 | 147548479 |
| chr11 | 118898588 | 118898638 |
| chr7 | 148144855 | 148144905 |
| chr7 | 148146989 | 148147039 |
| chr7 | 148310182 | 148310232 |
| chr7 | 148487590 | 148487640 |
| chr7 | 148675094 | 148675144 |
| chr7 | 148674992 | 148675042 |
| chr7 | 148733340 | 148733390 |
| chr7 | 148765802 | 148765852 |
| chr7 | 149081470 | 149081520 |
| chr7 | 149569249 | 149569299 |
| chr7 | 149731357 | 149731407 |
| chr7 | 149903958 | 149904008 |
| chr7 | 149914200 | 149914250 |
| chr7 | 150019757 | 150019807 |
| chr7 | 150681301 | 150681351 |
| chr7 | 150787128 | 150787178 |
| chr7 | 150811812 | 150811862 |
| chr7 | 150843521 | 150843571 |
| chr7 | 150845493 | 150845543 |
| chr7 | 150977859 | 150977909 |
| chr7 | 151030724 | 151030774 |
| chr7 | 151602852 | 151602902 |
| chr7 | 151603781 | 151603831 |
| chr7 | 151684647 | 151684697 |
| chr7 | 151733147 | 151733197 |
| chr7 | 151976637 | 151976687 |
| chr7 | 152151054 | 152151104 |
| chr11 | 119552407 | 119552457 |
| chr7 | 152190369 | 152190419 |
| chr7 | 152450839 | 152450889 |
| chr7 | 153192272 | 153192322 |
| chr7 | 153386640 | 153386690 |
| chr11 | 119641342 | 119641392 |
| chr7 | 153639294 | 153639344 |
| chr7 | 154082737 | 154082787 |
| chr7 | 154314260 | 154314310 |
| chr7 | 154445692 | 154445742 |
| chr7 | 154777339 | 154777389 |
| chr7 | 154796737 | 154796787 |
| chr7 | 155240895 | 155240945 |
| chr7 | 155402123 | 155402173 |
| chr7 | 155528292 | 155528342 |
| chr7 | 155529079 | 155529129 |
| chr7 | 156341430 | 156341480 |
| chr7 | 156497208 | 156497258 |
| chr11 | 120022883 | 120022933 |
| chr7 | 156587986 | 156588036 |
| chr7 | 156640825 | 156640875 |
| chr7 | 157162002 | 157162052 |
| chr7 | 157352150 | 157352200 |
| chr7 | 157512016 | 157512066 |
| chr7 | 157597151 | 157597201 |
| chr7 | 157641974 | 157642024 |
| chr7 | 157779570 | 157779620 |
| chr7 | 157819504 | 157819554 |
| chr11 | 120472979 | 120473029 |
| chr7 | 158362589 | 158362639 |
| chr7 | 158400516 | 158400566 |
| chr8 | 222053 | 222103 |
| chr8 | 600706 | 600756 |
| chr8 | 642793 | 642843 |
| chr8 | 763778 | 763828 |
| chr8 | 882970 | 883020 |
| chr11 | 120771603 | 120771653 |
| chr8 | 1668885 | 1668935 |
| chr8 | 1679785 | 1679835 |
| chr8 | 1942961 | 1943011 |
| chr8 | 2131193 | 2131243 |
| chr8 | 2149053 | 2149103 |
| chr8 | 2258865 | 2258915 |
| chr8 | 2517053 | 2517103 |
| chr8 | 2760276 | 2760326 |
| chr8 | 3280431 | 3280481 |
| chr8 | 3308114 | 3308164 |
| chr8 | 4137471 | 4137521 |
| chr8 | 4322321 | 4322371 |
| chr8 | 4322230 | 4322280 |
| chr8 | 4645500 | 4645550 |
| chr8 | 4727297 | 4727347 |
| chr8 | 4741697 | 4741747 |
| chr8 | 4885555 | 4885605 |
| chr8 | 5054762 | 5054812 |
| chr8 | 5075110 | 5075160 |
| chr8 | 5501858 | 5501908 |
| chr8 | 6059943 | 6059993 |
| chr8 | 6063590 | 6063640 |
| chr8 | 6144762 | 6144812 |
| chr8 | 6152670 | 6152720 |
| chr8 | 6385744 | 6385794 |
| chr10 | 18281274 | 18281324 |
| chr8 | 9425976 | 9426026 |
| chr8 | 9779830 | 9779880 |
| chr8 | 9896942 | 9896992 |
| chr8 | 9966883 | 9966933 |
| chr8 | 10166911 | 10166961 |
| chr8 | 10382226 | 10382276 |
| chr8 | 10382151 | 10382201 |
| chr8 | 10435924 | 10435974 |
| chr8 | 10528469 | 10528519 |
| chr8 | 10528296 | 10528346 |
| chr8 | 11235401 | 11235451 |
| chr8 | 11293986 | 11294036 |
| chr11 | 122277054 | 122277104 |
| chr8 | 11885993 | 11886043 |
| chr8 | 12446641 | 12446691 |
| chr8 | 13012225 | 13012275 |
| chr8 | 13448334 | 13448384 |
| chr8 | 13754370 | 13754420 |
| chr8 | 14045686 | 14045736 |
| chr8 | 14304097 | 14304147 |
| chr8 | 14347269 | 14347319 |
| chr8 | 14814171 | 14814221 |
| chr8 | 15273499 | 15273549 |
| chr8 | 15362080 | 15362130 |
| chr8 | 15517096 | 15517146 |
| chr8 | 15744955 | 15745005 |
| chr8 | 17286865 | 17286915 |
| chr8 | 18564018 | 18564068 |
| chr8 | 19676904 | 19676954 |
| chr8 | 20479433 | 20479483 |
| chr8 | 20742579 | 20742629 |
| chr8 | 20768318 | 20768368 |
| chr8 | 20935479 | 20935529 |
| chr8 | 22916330 | 22916380 |
| chr8 | 23215788 | 23215838 |
| chr8 | 23508115 | 23508165 |
| chr8 | 25001794 | 25001844 |
| chr8 | 26301614 | 26301664 |
| chr8 | 26320112 | 26320162 |
| chr8 | 26607969 | 26608019 |
| chr8 | 26975212 | 26975262 |
| chr8 | 26985023 | 26985073 |
| chr8 | 27363244 | 27363294 |
| chr10 | 18521043 | 18521093 |
| chr8 | 27916527 | 27916577 |
| chr8 | 27970116 | 27970166 |
| chr8 | 28305838 | 28305888 |
| chr8 | 28360924 | 28360974 |
| chr8 | 29367297 | 29367347 |
| chr8 | 30115657 | 30115707 |
| chr8 | 30115580 | 30115630 |
| chr8 | 30588058 | 30588108 |
| chr8 | 31224038 | 31224088 |
| chr8 | 33090738 | 33090788 |
| chr8 | 33100860 | 33100910 |
| chr8 | 34234841 | 34234891 |
| chr8 | 35836115 | 35836165 |
| chr8 | 36624434 | 36624484 |
| chr8 | 36870831 | 36870881 |
| chr8 | 37365374 | 37365424 |
| chr8 | 37365191 | 37365241 |
| chr8 | 38059585 | 38059635 |
| chr8 | 38099130 | 38099180 |
| chr8 | 38799145 | 38799195 |
| chr8 | 39303708 | 39303758 |
| chr8 | 39535845 | 39535895 |
| chr8 | 39637119 | 39637169 |
| chr8 | 39985205 | 39985255 |
| chr8 | 40224296 | 40224346 |
| chr8 | 40854397 | 40854447 |
| chr8 | 41432667 | 41432717 |
| chr8 | 42495133 | 42495183 |
| chr8 | 50201883 | 50201933 |
| chr8 | 50364187 | 50364237 |
| chr8 | 50563311 | 50563361 |
| chr8 | 51820559 | 51820609 |
| chr8 | 52101651 | 52101701 |
| chr8 | 52607316 | 52607366 |
| chr8 | 52623676 | 52623726 |
| chr8 | 52962599 | 52962649 |
| chr8 | 53517761 | 53517811 |
| chr8 | 53727703 | 53727753 |
| chr8 | 53807252 | 53807302 |
| chr11 | 125088469 | 125088519 |
| chr8 | 54248982 | 54249032 |
| chr8 | 54477211 | 54477261 |
| chr11 | 125176544 | 125176594 |
| chr8 | 55150706 | 55150756 |
| chr8 | 55262839 | 55262889 |
| chr8 | 56997960 | 56998010 |
| chr8 | 57682367 | 57682417 |
| chr8 | 57682246 | 57682296 |
| chr8 | 58054168 | 58054218 |
| chr8 | 58094509 | 58094559 |
| chr8 | 60104683 | 60104733 |
| chr8 | 61359882 | 61359932 |
| chr8 | 62785459 | 62785509 |
| chr8 | 63832572 | 63832622 |
| chr8 | 65434872 | 65434922 |
| chr8 | 65461858 | 65461908 |
| chr8 | 66631702 | 66631752 |
| chr8 | 68364232 | 68364282 |
| chr8 | 69284985 | 69285035 |
| chr8 | 70803893 | 70803943 |
| chr8 | 70844439 | 70844489 |
| chr8 | 71050809 | 71050859 |
| chr8 | 71066722 | 71066772 |
| chr8 | 72143136 | 72143186 |
| chr8 | 74147741 | 74147791 |
| chr8 | 75077701 | 75077751 |
| chr8 | 77990898 | 77990948 |
| chr8 | 78778534 | 78778584 |
| chr8 | 78786789 | 78786839 |
| chr8 | 79443201 | 79443251 |
| chr8 | 80272702 | 80272752 |
| chr8 | 80322557 | 80322607 |
| chr8 | 81671735 | 81671785 |
| chr8 | 83052881 | 83052931 |
| chr8 | 84945126 | 84945176 |
| chr8 | 86221696 | 86221746 |
| chr8 | 87759115 | 87759165 |
| chr8 | 91618476 | 91618526 |
| chr8 | 92064428 | 92064478 |
| chr8 | 92394805 | 92394855 |
| chr8 | 93178573 | 93178623 |
| chr8 | 94534426 | 94534476 |
| chr8 | 95268153 | 95268203 |
| chr8 | 95804760 | 95804810 |
| chr8 | 96270710 | 96270760 |
| chr8 | 98264369 | 98264419 |
| chr8 | 98818862 | 98818912 |
| chr8 | 98818607 | 98818657 |
| chr8 | 99725932 | 99725982 |
| chr8 | 101171581 | 101171631 |
| chr8 | 101723109 | 101723159 |
| chr8 | 101810193 | 101810243 |
| chr8 | 101877391 | 101877441 |
| chr8 | 101982903 | 101982953 |
| chr8 | 102953051 | 102953101 |
| chr8 | 103101496 | 103101546 |
| chr8 | 103140451 | 103140501 |
| chr8 | 103410341 | 103410391 |
| chr8 | 103450798 | 103450848 |
| chr8 | 103503724 | 103503774 |
| chr8 | 103545959 | 103546009 |
| chr8 | 103898175 | 103898225 |
| chr8 | 103963068 | 103963118 |
| chr8 | 104438989 | 104439039 |
| chr8 | 105361557 | 105361607 |
| chr8 | 106409646 | 106409696 |
| chr8 | 107029534 | 107029584 |
| chr8 | 107371879 | 107371929 |
| chr8 | 107546488 | 107546538 |
| chr8 | 108248769 | 108248819 |
| chr8 | 109800655 | 109800705 |
| chr8 | 109922902 | 109922952 |
| chr8 | 111361310 | 111361360 |
| chr8 | 112133361 | 112133411 |
| chr8 | 112817743 | 112817793 |
| chr8 | 113432643 | 113432693 |
| chr8 | 113724457 | 113724507 |
| chr8 | 115782273 | 115782323 |
| chr8 | 117587957 | 117588007 |
| chr8 | 119455252 | 119455302 |
| chr8 | 119517334 | 119517384 |
| chr11 | 128899266 | 128899316 |
| chr8 | 119657936 | 119657986 |
| chr11 | 128981992 | 128982042 |
| chr8 | 120798624 | 120798674 |
| chr8 | 120939691 | 120939741 |
| chr8 | 121458309 | 121458359 |
| chr8 | 122480079 | 122480129 |
| chr8 | 122699189 | 122699239 |
| chr8 | 123895484 | 123895534 |
| chr8 | 124150828 | 124150878 |
| chr8 | 124527780 | 124527830 |
| chr8 | 124595224 | 124595274 |
| chr8 | 124686598 | 124686648 |
| chr8 | 124927652 | 124927702 |
| chr8 | 125140793 | 125140843 |
| chr8 | 125440377 | 125440427 |
| chr8 | 126551628 | 126551678 |
| chr8 | 126583373 | 126583423 |
| chr8 | 126828689 | 126828739 |
| chr8 | 127261747 | 127261797 |
| chr8 | 127366210 | 127366260 |
| chr8 | 127765825 | 127765875 |
| chr8 | 128103604 | 128103654 |
| chr8 | 128725128 | 128725178 |
| chr11 | 129426885 | 129426935 |
| chr8 | 129953432 | 129953482 |
| chr8 | 130442799 | 130442849 |
| chr8 | 133637696 | 133637746 |
| chr11 | 129580251 | 129580301 |
| chr8 | 134741678 | 134741728 |
| chr11 | 129585425 | 129585475 |
| chr8 | 134906662 | 134906712 |
| chr11 | 129598235 | 129598285 |
| chr11 | 129599652 | 129599702 |
| chr8 | 137050433 | 137050483 |
| chr8 | 137427450 | 137427500 |
| chr8 | 137513504 | 137513554 |
| chr8 | 137520868 | 137520918 |
| chr8 | 139143153 | 139143203 |
| chr8 | 139246315 | 139246365 |
| chr8 | 139920986 | 139921036 |
| chr8 | 140308834 | 140308884 |
| chr8 | 140357648 | 140357698 |
| chr8 | 140388946 | 140388996 |
| chr8 | 140438957 | 140439007 |
| chr11 | 129786485 | 129786535 |
| chr8 | 141051202 | 141051252 |
| chr8 | 141484094 | 141484144 |
| chr8 | 141488360 | 141488410 |
| chr8 | 142001483 | 142001533 |
| chr8 | 142369390 | 142369440 |
| chr8 | 142540071 | 142540121 |
| chr8 | 142569428 | 142569478 |
| chr8 | 142971896 | 142971946 |
| chr8 | 142980516 | 142980566 |
| chr8 | 142988065 | 142988115 |
| chr8 | 143071155 | 143071205 |
| chr8 | 143100617 | 143100667 |
| chr8 | 143129391 | 143129441 |
| chr8 | 143586889 | 143586939 |
| chr8 | 144295522 | 144295572 |
| chr8 | 144352829 | 144352879 |
| chr8 | 144469340 | 144469390 |
| chr8 | 144852594 | 144852644 |
| chr8 | 144862249 | 144862299 |
| chr8 | 144932498 | 144932548 |
| chr8 | 144933507 | 144933557 |
| chr8 | 144943066 | 144943116 |
| chr8 | 145030094 | 145030144 |
| chr8 | 145088937 | 145088987 |
| chr8 | 145092772 | 145092822 |
| chr8 | 145093862 | 145093912 |
| chr11 | 130955029 | 130955079 |
| chr8 | 145782579 | 145782629 |
| chr8 | 145782514 | 145782564 |
| chr8 | 145944388 | 145944438 |
| chr8 | 146054993 | 146055043 |
| chr9 | 2245436 | 2245486 |
| chr9 | 2245219 | 2245269 |
| chr9 | 3584098 | 3584148 |
| chr9 | 4875529 | 4875579 |
| chr9 | 5428906 | 5428956 |
| chr9 | 6085716 | 6085766 |
| chr9 | 6349751 | 6349801 |
| chr9 | 6697116 | 6697166 |
| chr9 | 6713465 | 6713515 |
| chr9 | 6718490 | 6718540 |
| chr9 | 7430125 | 7430175 |
| chr9 | 10624300 | 10624350 |
| chr9 | 10777000 | 10777050 |
| chr9 | 12371070 | 12371120 |
| chr9 | 12994268 | 12994318 |
| chr10 | 19936528 | 19936578 |
| chr9 | 14832661 | 14832711 |
| chr9 | 15338085 | 15338135 |
| chr9 | 15523219 | 15523269 |
| chr9 | 16134469 | 16134519 |
| chr9 | 17023371 | 17023421 |
| chr9 | 17119327 | 17119377 |
| chr9 | 19888423 | 19888473 |
| chr9 | 19955092 | 19955142 |
| chr9 | 20504875 | 20504925 |
| chr9 | 20815899 | 20815949 |
| chr9 | 20815816 | 20815866 |
| chr9 | 21971252 | 21971302 |
| chr11 | 132784548 | 132784598 |
| chr9 | 23488280 | 23488330 |
| chr9 | 23964563 | 23964613 |
| chr9 | 24714757 | 24714807 |
| chr11 | 132923956 | 132924006 |
| chr9 | 26595158 | 26595208 |
| chr9 | 28426844 | 28426894 |
| chr9 | 29502206 | 29502256 |
| chr9 | 30172183 | 30172233 |
| chr9 | 30288992 | 30289042 |
| chr9 | 30290641 | 30290691 |
| chr9 | 30550998 | 30551048 |
| chr9 | 30678947 | 30678997 |
| chr11 | 133048199 | 133048249 |
| chr9 | 33189448 | 33189498 |
| chr9 | 33207627 | 33207677 |
| chr9 | 33277888 | 33277938 |
| chr9 | 34152669 | 34152719 |
| chr9 | 34523161 | 34523211 |
| chr9 | 34553697 | 34553747 |
| chr9 | 34553414 | 34553464 |
| chr9 | 34667678 | 34667728 |
| chr9 | 34669728 | 34669778 |
| chr9 | 34929406 | 34929456 |
| chr9 | 35619948 | 35619998 |
| chr9 | 35848570 | 35848620 |
| chr9 | 36552123 | 36552173 |
| chr9 | 36552755 | 36552805 |
| chr9 | 36810686 | 36810736 |
| chr9 | 36890298 | 36890348 |
| chr9 | 37056029 | 37056079 |
| chr9 | 37351474 | 37351524 |
| chr9 | 38221307 | 38221357 |
| chr9 | 38235772 | 38235822 |
| chr9 | 38880245 | 38880295 |
| chr9 | 40757045 | 40757095 |
| chr9 | 41246676 | 41246726 |
| chr9 | 42163936 | 42163986 |
| chr9 | 42639813 | 42639863 |
| chr9 | 63063579 | 63063629 |
| chr9 | 66581964 | 66582014 |
| chr9 | 67095389 | 67095439 |
| chr9 | 67341341 | 67341391 |
| chr9 | 67714503 | 67714553 |
| chr9 | 68221395 | 68221445 |
| chr9 | 68498191 | 68498241 |
| chr9 | 68566016 | 68566066 |
| chr9 | 69133093 | 69133143 |
| chr9 | 69633208 | 69633258 |
| chr9 | 70066017 | 70066067 |
| chr9 | 70108524 | 70108574 |
| chr9 | 70579246 | 70579296 |
| chr9 | 70798044 | 70798094 |
| chr9 | 71191888 | 71191938 |
| chr9 | 73311792 | 73311842 |
| chr9 | 73352841 | 73352891 |
| chr9 | 73935023 | 73935073 |
| chr9 | 74496742 | 74496792 |
| chr9 | 74554257 | 74554307 |
| chr9 | 75018922 | 75018972 |
| chr9 | 75026755 | 75026805 |
| chr9 | 75105168 | 75105218 |
| chr9 | 76145955 | 76146005 |
| chr9 | 76145889 | 76145939 |
| chr9 | 76603008 | 76603058 |
| chr9 | 76764862 | 76764912 |
| chr9 | 76833828 | 76833878 |
| chr9 | 78047415 | 78047465 |
| chr9 | 78047267 | 78047317 |
| chr9 | 79383344 | 79383394 |
| chr9 | 79538516 | 79538566 |
| chr9 | 79845235 | 79845285 |
| chr9 | 79865181 | 79865231 |
| chr9 | 81792771 | 81792821 |
| chr9 | 82892080 | 82892130 |
| chr9 | 83906444 | 83906494 |
| chr9 | 84191694 | 84191744 |
| chr9 | 84809835 | 84809885 |
| chr9 | 85170335 | 85170385 |
| chr9 | 85242521 | 85242571 |
| chr9 | 85242834 | 85242884 |
| chr9 | 85243091 | 85243141 |
| chr9 | 85511805 | 85511855 |
| chr9 | 85545407 | 85545457 |
| chr9 | 85545711 | 85545761 |
| chr9 | 85546215 | 85546265 |
| chr9 | 85546291 | 85546341 |
| chr9 | 85826471 | 85826521 |
| chr9 | 86137877 | 86137927 |
| chr9 | 86137286 | 86137336 |
| chr11 | 133795110 | 133795160 |
| chr9 | 86311119 | 86311169 |
| chr9 | 86364222 | 86364272 |
| chr9 | 86380195 | 86380245 |
| chr9 | 86433163 | 86433213 |
| chr9 | 86471583 | 86471633 |
| chr9 | 86829649 | 86829699 |
| chr9 | 87075948 | 87075998 |
| chr9 | 87113565 | 87113615 |
| chr9 | 87359535 | 87359585 |
| chr9 | 87359651 | 87359701 |
| chr9 | 87390311 | 87390361 |
| chr9 | 88238121 | 88238171 |
| chr9 | 88522155 | 88522205 |
| chr9 | 88522340 | 88522390 |
| chr9 | 89039415 | 89039465 |
| chr9 | 89068793 | 89068843 |
| chr9 | 89069202 | 89069252 |
| chr9 | 89069301 | 89069351 |
| chr9 | 89539882 | 89539932 |
| chr9 | 89540317 | 89540367 |
| chr9 | 89540656 | 89540706 |
| chr9 | 90092279 | 90092329 |
| chr9 | 90107961 | 90108011 |
| chr11 | 134006057 | 134006107 |
| chr9 | 90753398 | 90753448 |
| chr9 | 90786194 | 90786244 |
| chr9 | 90882541 | 90882591 |
| chr9 | 90957962 | 90958012 |
| chr9 | 91152460 | 91152510 |
| chr11 | 134136562 | 134136612 |
| chr9 | 91721936 | 91721986 |
| chr9 | 92073065 | 92073115 |
| chr9 | 92072981 | 92073031 |
| chr9 | 92311028 | 92311078 |
| chr9 | 92389776 | 92389826 |
| chr11 | 134251212 | 134251262 |
| chr11 | 134289347 | 134289397 |
| chr9 | 92843421 | 92843471 |
| chr9 | 92843555 | 92843605 |
| chr9 | 93417016 | 93417066 |
| chr9 | 93537821 | 93537871 |
| chr9 | 93538074 | 93538124 |
| chr9 | 93538354 | 93538404 |
| chr9 | 93737080 | 93737130 |
| chr9 | 93746844 | 93746894 |
| chr9 | 93811988 | 93812038 |
| chr9 | 94173045 | 94173095 |
| chr9 | 94429393 | 94429443 |
| chr9 | 94803958 | 94804008 |
| chr9 | 94830443 | 94830493 |
| chr9 | 95021737 | 95021787 |
| chr9 | 95171099 | 95171149 |
| chr9 | 95216987 | 95217037 |
| chr9 | 95884785 | 95884835 |
| chr9 | 95938036 | 95938086 |
| chr9 | 96516831 | 96516881 |
| chr9 | 96587287 | 96587337 |
| chr9 | 96775847 | 96775897 |
| chr12 | 318271 | 318321 |
| chr9 | 97892216 | 97892266 |
| chr12 | 405938 | 405988 |
| chr12 | 406692 | 406742 |
| chr9 | 101483001 | 101483051 |
| chr9 | 102344332 | 102344382 |
| chr9 | 103142441 | 103142491 |
| chr9 | 103657271 | 103657321 |
| chr9 | 103838681 | 103838731 |
| chr9 | 104021084 | 104021134 |
| chr9 | 104862008 | 104862058 |
| chr9 | 105413194 | 105413244 |
| chr9 | 105476092 | 105476142 |
| chr9 | 105476006 | 105476056 |
| chr9 | 105489568 | 105489618 |
| chr9 | 105794629 | 105794679 |
| chr9 | 106491175 | 106491225 |
| chr9 | 109266936 | 109266986 |
| chr9 | 109597624 | 109597674 |
| chr12 | 688030 | 688080 |
| chr9 | 109676769 | 109676819 |
| chr9 | 109999677 | 109999727 |
| chr9 | 110093480 | 110093530 |
| chr9 | 110448133 | 110448183 |
| chr9 | 110517709 | 110517759 |
| chr9 | 110878440 | 110878490 |
| chr9 | 111048161 | 111048211 |
| chr9 | 111117436 | 111117486 |
| chr9 | 112452670 | 112452720 |
| chr9 | 112700396 | 112700446 |
| chr9 | 113485125 | 113485175 |
| chr12 | 957552 | 957602 |
| chr9 | 115102582 | 115102632 |
| chr9 | 115990107 | 115990157 |
| chr9 | 116162600 | 116162650 |
| chr12 | 1000758 | 1000808 |
| chr9 | 116907025 | 116907075 |
| chr9 | 118334246 | 118334296 |
| chr9 | 118980524 | 118980574 |
| chr9 | 119026190 | 119026240 |
| chr9 | 120491289 | 120491339 |
| chr9 | 122256542 | 122256592 |
| chr9 | 122283118 | 122283168 |
| chr9 | 122342242 | 122342292 |
| chr9 | 122764946 | 122764996 |
| chr9 | 122984062 | 122984112 |
| chr9 | 123538221 | 123538271 |
| chr9 | 123751882 | 123751932 |
| chr9 | 124153996 | 124154046 |
| chr9 | 124714204 | 124714254 |
| chr9 | 124913391 | 124913441 |
| chr9 | 124977261 | 124977311 |
| chr9 | 125035429 | 125035479 |
| chr9 | 125037159 | 125037209 |
| chr9 | 125043462 | 125043512 |
| chr9 | 125046392 | 125046442 |
| chr10 | 21551012 | 21551062 |
| chr9 | 125996229 | 125996279 |
| chr9 | 126642479 | 126642529 |
| chr9 | 126685267 | 126685317 |
| chr9 | 126998275 | 126998325 |
| chr9 | 126998807 | 126998857 |
| chr9 | 127190731 | 127190781 |
| chr9 | 127453751 | 127453801 |
| chr9 | 127906594 | 127906644 |
| chr9 | 128100286 | 128100336 |
| chr9 | 129131362 | 129131412 |
| chr9 | 129152844 | 129152894 |
| chr9 | 129155825 | 129155875 |
| chr9 | 129179777 | 129179827 |
| chr9 | 129539132 | 129539182 |
| chr9 | 129925169 | 129925219 |
| chr9 | 130629963 | 130630013 |
| chr9 | 131005886 | 131005936 |
| chr9 | 131465815 | 131465865 |
| chr9 | 131963270 | 131963320 |
| chr9 | 132024899 | 132024949 |
| chr9 | 132082816 | 132082866 |
| chr9 | 132141966 | 132142016 |
| chr9 | 132217851 | 132217901 |
| chr9 | 132317874 | 132317924 |
| chr9 | 132580615 | 132580665 |
| chr9 | 132625264 | 132625314 |
| chr9 | 132825398 | 132825448 |
| chr9 | 132825261 | 132825311 |
| chr9 | 132828175 | 132828225 |
| chr9 | 132865415 | 132865465 |
| chr9 | 132878846 | 132878896 |
| chr9 | 132894599 | 132894649 |
| chr9 | 133596915 | 133596965 |
| chr9 | 133622612 | 133622662 |
| chr9 | 133657488 | 133657538 |
| chr9 | 133711537 | 133711587 |
| chr9 | 133786668 | 133786718 |
| chr9 | 134472238 | 134472288 |
| chr9 | 134612272 | 134612322 |
| chr9 | 134744747 | 134744797 |
| chr9 | 134843242 | 134843292 |
| chr9 | 134960777 | 134960827 |
| chr9 | 135059499 | 135059549 |
| chr9 | 135375222 | 135375272 |
| chr9 | 135618992 | 135619042 |
| chr9 | 136062331 | 136062381 |
| chr9 | 136153727 | 136153777 |
| chr9 | 136175563 | 136175613 |
| chr9 | 136175297 | 136175347 |
| chr12 | 3316335 | 3316385 |
| chr12 | 3316088 | 3316138 |
| chr12 | 3347608 | 3347658 |
| chr12 | 3688143 | 3688193 |
| chr12 | 3744503 | 3744553 |
| chr12 | 5012417 | 5012467 |
| chr12 | 5162959 | 5163009 |
| chr12 | 5597144 | 5597194 |
| chr10 | 21923931 | 21923981 |
| chr12 | 6218167 | 6218217 |
| chr12 | 6385303 | 6385353 |
| chr12 | 6774751 | 6774801 |
| chr12 | 7008041 | 7008091 |
| chr12 | 7812778 | 7812828 |
| chr12 | 7816026 | 7816076 |
| chr12 | 7994516 | 7994566 |
| chr12 | 8040415 | 8040465 |
| chr12 | 8043462 | 8043512 |
| chr12 | 8157881 | 8157931 |
| chr12 | 8829758 | 8829808 |
| chr12 | 8857636 | 8857686 |
| chr12 | 9571640 | 9571690 |
| chr12 | 9920495 | 9920545 |
| chr12 | 10349704 | 10349754 |
| chr12 | 10349640 | 10349690 |
| chr12 | 10439787 | 10439837 |
| chr12 | 10469448 | 10469498 |
| chr12 | 11027207 | 11027257 |
| chr12 | 11026988 | 11027038 |
| chr12 | 11681673 | 11681723 |
| chr12 | 11757176 | 11757226 |
| chr12 | 12388569 | 12388619 |
| chr12 | 12635361 | 12635411 |
| chr10 | 23021506 | 23021556 |
| chr12 | 12790298 | 12790348 |
| chr12 | 13065507 | 13065557 |
| chr12 | 13420931 | 13420981 |
| chr12 | 13420842 | 13420892 |
| chr12 | 13922435 | 13922485 |
| chr12 | 14259273 | 14259323 |
| chr12 | 14267124 | 14267174 |
| chr12 | 14318497 | 14318547 |
| chr12 | 14342962 | 14343012 |
| chr12 | 14348933 | 14348983 |
| chr12 | 14374523 | 14374573 |
| chr12 | 15213662 | 15213712 |
| chr12 | 15213390 | 15213440 |
| chr12 | 16166769 | 16166819 |
| chr12 | 17013608 | 17013658 |
| chr12 | 17312702 | 17312752 |
| chr12 | 18164944 | 18164994 |
| chr12 | 18679440 | 18679490 |
| chr12 | 19661140 | 19661190 |
| chr12 | 20373243 | 20373293 |
| chr12 | 20840745 | 20840795 |
| chr12 | 21523399 | 21523449 |
| chr12 | 21539904 | 21539954 |
| chr12 | 21780820 | 21780870 |
| chr12 | 22816623 | 22816673 |
| chr12 | 22979813 | 22979863 |
| chr10 | 23979172 | 23979222 |
| chr12 | 24333937 | 24333987 |
| chr12 | 24758090 | 24758140 |
| chr12 | 25496369 | 25496419 |
| chr12 | 25923222 | 25923272 |
| chr12 | 26929078 | 26929128 |
| chr12 | 27106909 | 27106959 |
| chr12 | 27801210 | 27801260 |
| chr12 | 29142978 | 29143028 |
| chr12 | 30194074 | 30194124 |
| chr12 | 30195823 | 30195873 |
| chr12 | 30404482 | 30404532 |
| chr12 | 31294626 | 31294676 |
| chr12 | 31688673 | 31688723 |
| chr12 | 31767862 | 31767912 |
| chr12 | 32046203 | 32046253 |
| chr12 | 32111104 | 32111154 |
| chr12 | 32228280 | 32228330 |
| chr12 | 32510748 | 32510798 |
| chr12 | 32800570 | 32800620 |
| chr10 | 24842317 | 24842367 |
| chr10 | 24862203 | 24862253 |
| chr12 | 37635437 | 37635487 |
| chr12 | 37966202 | 37966252 |
| chr12 | 38059889 | 38059939 |
| chr12 | 38812794 | 38812844 |
| chr12 | 40264484 | 40264534 |
| chr12 | 40423906 | 40423956 |
| chr12 | 41933335 | 41933385 |
| chr12 | 42314242 | 42314292 |
| chr12 | 45948540 | 45948590 |
| chr10 | 25588631 | 25588681 |
| chr12 | 46995509 | 46995559 |
| chr12 | 47066864 | 47066914 |
| chr12 | 47261108 | 47261158 |
| chr10 | 25931911 | 25931961 |
| chr12 | 47580608 | 47580658 |
| chr12 | 47758813 | 47758863 |
| chr12 | 47837884 | 47837934 |
| chr12 | 47892752 | 47892802 |
| chr12 | 48757183 | 48757233 |
| chr12 | 49002379 | 49002429 |
| chr12 | 49438886 | 49438936 |
| chr12 | 50813925 | 50813975 |
| chr12 | 50843388 | 50843438 |
| chr12 | 51192764 | 51192814 |
| chr12 | 51517665 | 51517715 |
| chr12 | 51517349 | 51517399 |
| chr10 | 1160994 | 1161044 |
| chr12 | 54160277 | 54160327 |
| chr10 | 27269703 | 27269753 |
| chr12 | 54561454 | 54561504 |
| chr12 | 54946744 | 54946794 |
| chr12 | 56236684 | 56236734 |
| chr12 | 56358586 | 56358636 |
| chr12 | 56548124 | 56548174 |
| chr10 | 27721240 | 27721290 |
| chr12 | 56776455 | 56776505 |
| chr12 | 57437494 | 57437544 |
| chr12 | 57963795 | 57963845 |
| chr12 | 58910945 | 58910995 |
| chr12 | 59251633 | 59251683 |
| chr12 | 60206171 | 60206221 |
| chr10 | 27941492 | 27941542 |
| chr10 | 27948562 | 27948612 |
| chr12 | 62223360 | 62223410 |
| chr12 | 62418410 | 62418460 |
| chr12 | 62865265 | 62865315 |
| chr12 | 63200484 | 63200534 |
| chr12 | 63239144 | 63239194 |
| chr12 | 63451171 | 63451221 |
| chr12 | 65813316 | 65813366 |
| chr12 | 66208371 | 66208421 |
| chr12 | 66321174 | 66321224 |
| chr12 | 67020255 | 67020305 |
| chr12 | 67810793 | 67810843 |
| chr12 | 67837052 | 67837102 |
| chr10 | 28681573 | 28681623 |
| chr12 | 68806643 | 68806693 |
| chr10 | 28707379 | 28707429 |
| chr12 | 71383160 | 71383210 |
| chr10 | 28878208 | 28878258 |
| chr12 | 73380290 | 73380340 |
| chr12 | 74195049 | 74195099 |
| chr12 | 74703598 | 74703648 |
| chr12 | 75400118 | 75400168 |
| chr12 | 75785476 | 75785526 |
| chr12 | 75955923 | 75955973 |
| chr12 | 78349239 | 78349289 |
| chr12 | 78988873 | 78988923 |
| chr10 | 29120249 | 29120299 |
| chr12 | 81226341 | 81226391 |
| chr12 | 81519889 | 81519939 |
| chr12 | 82084359 | 82084409 |
| chr12 | 82119256 | 82119306 |
| chr12 | 82968880 | 82968930 |
| chr12 | 83201254 | 83201304 |
| chr12 | 83466065 | 83466115 |
| chr12 | 83543595 | 83543645 |
| chr12 | 84515765 | 84515815 |
| chr12 | 85047542 | 85047592 |
| chr12 | 85414207 | 85414257 |
| chr12 | 86591717 | 86591767 |
| chr12 | 87803914 | 87803964 |
| chr12 | 88184808 | 88184858 |
| chr12 | 88670035 | 88670085 |
| chr12 | 89113498 | 89113548 |
| chr12 | 89198972 | 89199022 |
| chr12 | 91776408 | 91776458 |
| chr12 | 91973235 | 91973285 |
| chr12 | 91978491 | 91978541 |
| chr12 | 92049543 | 92049593 |
| chr10 | 29740189 | 29740239 |
| chr12 | 94191683 | 94191733 |
| chr12 | 94981437 | 94981487 |
| chr12 | 96179753 | 96179803 |
| chr12 | 97345968 | 97346018 |
| chr12 | 97377877 | 97377927 |
| chr12 | 97484799 | 97484849 |
| chr12 | 97505100 | 97505150 |
| chr12 | 98466399 | 98466449 |
| chr10 | 29877664 | 29877714 |
| chr12 | 99125710 | 99125760 |
| chr12 | 100586100 | 100586150 |
| chr10 | 30003275 | 30003325 |
| chr12 | 102529369 | 102529419 |
| chr12 | 102687288 | 102687338 |
| chr12 | 103065833 | 103065883 |
| chr12 | 103070260 | 103070310 |
| chr12 | 104506203 | 104506253 |
| chr12 | 105073461 | 105073511 |
| chr12 | 106314005 | 106314055 |
| chr12 | 106399083 | 106399133 |
| chr12 | 106446605 | 106446655 |
| chr12 | 106856858 | 106856908 |
| chr12 | 107316726 | 107316776 |
| chr12 | 107323852 | 107323902 |
| chr12 | 107371584 | 107371634 |
| chr12 | 107455957 | 107456007 |
| chr12 | 108140379 | 108140429 |
| chr10 | 30588537 | 30588587 |
| chr12 | 108975971 | 108976021 |
| chr12 | 108980742 | 108980792 |
| chr12 | 109084551 | 109084601 |
| chr12 | 109686990 | 109687040 |
| chr10 | 30939310 | 30939360 |
| chr12 | 110579689 | 110579739 |
| chr12 | 110579530 | 110579580 |
| chr12 | 111435179 | 111435229 |
| chr12 | 112169280 | 112169330 |
| chr12 | 112297555 | 112297605 |
| chr10 | 31180246 | 31180296 |
| chr12 | 113394593 | 113394643 |
| chr10 | 31252721 | 31252771 |
| chr12 | 113868927 | 113868977 |
| chr12 | 114331616 | 114331666 |
| chr12 | 114444554 | 114444604 |
| chr12 | 115444209 | 115444259 |
| chr12 | 115592315 | 115592365 |
| chr12 | 115631343 | 115631393 |
| chr12 | 116070032 | 116070082 |
| chr12 | 116069773 | 116069823 |
| chr12 | 116078825 | 116078875 |
| chr12 | 116200471 | 116200521 |
| chr12 | 116219917 | 116219967 |
| chr12 | 117370076 | 117370126 |
| chr12 | 118434808 | 118434858 |
| chr12 | 118796113 | 118796163 |
| chr12 | 118875135 | 118875185 |
| chr12 | 119153768 | 119153818 |
| chr12 | 119155412 | 119155462 |
| chr12 | 119458129 | 119458179 |
| chr12 | 119478454 | 119478504 |
| chr10 | 32361883 | 32361933 |
| chr12 | 119781237 | 119781287 |
| chr12 | 119809664 | 119809714 |
| chr12 | 119882504 | 119882554 |
| chr12 | 119978205 | 119978255 |
| chr12 | 120123989 | 120124039 |
| chr12 | 120123865 | 120123915 |
| chr12 | 120213417 | 120213467 |
| chr12 | 120527391 | 120527441 |
| chr12 | 120697937 | 120697987 |
| chr12 | 121088873 | 121088923 |
| chr12 | 121157921 | 121157971 |
| chr12 | 121157804 | 121157854 |
| chr10 | 32731823 | 32731873 |
| chr12 | 121901610 | 121901660 |
| chr12 | 122381037 | 122381087 |
| chr12 | 122730405 | 122730455 |
| chr12 | 122730225 | 122730275 |
| chr12 | 123029942 | 123029992 |
| chr12 | 123065871 | 123065921 |
| chr12 | 123144760 | 123144810 |
| chr12 | 123266500 | 123266550 |
| chr12 | 123267038 | 123267088 |
| chr12 | 123484351 | 123484401 |
| chr12 | 123629231 | 123629281 |
| chr12 | 123813227 | 123813277 |
| chr12 | 124272184 | 124272234 |
| chr12 | 124332821 | 124332871 |
| chr12 | 124800791 | 124800841 |
| chr10 | 33874905 | 33874955 |
| chr12 | 125529778 | 125529828 |
| chr12 | 125579367 | 125579417 |
| chr12 | 125579180 | 125579230 |
| chr12 | 125738149 | 125738199 |
| chr12 | 125835508 | 125835558 |
| chr12 | 126051151 | 126051201 |
| chr12 | 126073651 | 126073701 |
| chr12 | 126536806 | 126536856 |
| chr12 | 126857744 | 126857794 |
| chr12 | 127043789 | 127043839 |
| chr12 | 127367738 | 127367788 |
| chr12 | 127492584 | 127492634 |
| chr12 | 127686575 | 127686625 |
| chr12 | 127772185 | 127772235 |
| chr12 | 127947487 | 127947537 |
| chr12 | 127991053 | 127991103 |
| chr12 | 127990901 | 127990951 |
| chr12 | 128132968 | 128133018 |
| chr10 | 34600230 | 34600280 |
| chr12 | 128148527 | 128148577 |
| chr12 | 128164711 | 128164761 |
| chr12 | 128413797 | 128413847 |
| chr12 | 128660138 | 128660188 |
| chr12 | 128773601 | 128773651 |
| chr12 | 128921263 | 128921313 |
| chr12 | 129292767 | 129292817 |
| chr12 | 129347749 | 129347799 |
| chr12 | 129349109 | 129349159 |
| chr10 | 34899259 | 34899309 |
| chr12 | 129451073 | 129451123 |
| chr10 | 34951328 | 34951378 |
| chr12 | 129598866 | 129598916 |
| chr12 | 129642208 | 129642258 |
| chr12 | 129762809 | 129762859 |
| chr12 | 129967973 | 129968023 |
| chr12 | 129968174 | 129968224 |
| chr12 | 129968741 | 129968791 |
| chr12 | 130131599 | 130131649 |
| chr12 | 130205541 | 130205591 |
| chr12 | 130208868 | 130208918 |
| chr12 | 130294882 | 130294932 |
| chr10 | 35159995 | 35160045 |
| chr12 | 130645668 | 130645718 |
| chr12 | 130653314 | 130653364 |
| chr12 | 130653142 | 130653192 |
| chr12 | 131193638 | 131193688 |
| chr12 | 131393291 | 131393341 |
| chr12 | 131715407 | 131715457 |
| chr12 | 131755718 | 131755768 |
| chr12 | 131757097 | 131757147 |
| chr12 | 131758360 | 131758410 |
| chr13 | 17464102 | 17464152 |
| chr13 | 18282043 | 18282093 |
| chr13 | 18900030 | 18900080 |
| chr13 | 19768005 | 19768055 |
| chr13 | 19849293 | 19849343 |
| chr13 | 19871366 | 19871416 |
| chr13 | 20022545 | 20022595 |
| chr13 | 21383852 | 21383902 |
| chr10 | 35962120 | 35962170 |
| chr13 | 21870532 | 21870582 |
| chr10 | 36009656 | 36009706 |
| chr13 | 22509300 | 22509350 |
| chr13 | 23453886 | 23453936 |
| chr13 | 23524887 | 23524937 |
| chr13 | 23524699 | 23524749 |
| chr13 | 23826711 | 23826761 |
| chr13 | 24576184 | 24576234 |
| chr13 | 25001165 | 25001215 |
| chr13 | 25407090 | 25407140 |
| chr13 | 25460577 | 25460627 |
| chr13 | 25863440 | 25863490 |
| chr13 | 25892907 | 25892957 |
| chr13 | 26311853 | 26311903 |
| chr10 | 36887200 | 36887250 |
| chr13 | 27003130 | 27003180 |
| chr13 | 27177944 | 27177994 |
| chr13 | 28122798 | 28122848 |
| chr13 | 28702600 | 28702650 |
| chr10 | 37023962 | 37024012 |
| chr13 | 29235435 | 29235485 |
| chr13 | 30843630 | 30843680 |
| chr13 | 31245825 | 31245875 |
| chr10 | 37167941 | 37167991 |
| chr13 | 32432318 | 32432368 |
| chr13 | 35518883 | 35518933 |
| chr13 | 35518732 | 35518782 |
| chr13 | 36558783 | 36558833 |
| chr13 | 36683426 | 36683476 |
| chr13 | 39615560 | 39615610 |
| chr13 | 40941370 | 40941420 |
| chr10 | 38084503 | 38084553 |
| chr13 | 42288179 | 42288229 |
| chr13 | 42788680 | 42788730 |
| chr13 | 43245176 | 43245226 |
| chr13 | 43497740 | 43497790 |
| chr13 | 43755945 | 43755995 |
| chr13 | 43756307 | 43756357 |
| chr13 | 44051655 | 44051705 |
| chr10 | 38381296 | 38381346 |
| chr13 | 44490054 | 44490104 |
| chr13 | 44489840 | 44489890 |
| chr13 | 44828104 | 44828154 |
| chr13 | 45972817 | 45972867 |
| chr13 | 46218125 | 46218175 |
| chr13 | 47159690 | 47159740 |
| chr13 | 47300569 | 47300619 |
| chr13 | 48133222 | 48133272 |
| chr13 | 48276674 | 48276724 |
| chr13 | 49347648 | 49347698 |
| chr10 | 41968502 | 41968552 |
| chr10 | 42034027 | 42034077 |
| chr13 | 51209007 | 51209057 |
| chr13 | 51984138 | 51984188 |
| chr13 | 52661975 | 52662025 |
| chr13 | 52748659 | 52748709 |
| chr13 | 53109958 | 53110008 |
| chr13 | 53252317 | 53252367 |
| chr13 | 53573421 | 53573471 |
| chr13 | 54005863 | 54005913 |
| chr13 | 54027032 | 54027082 |
| chr13 | 54033072 | 54033122 |
| chr13 | 58036223 | 58036273 |
| chr13 | 58869201 | 58869251 |
| chr13 | 58965679 | 58965729 |
| chr13 | 59027297 | 59027347 |
| chr13 | 59108148 | 59108198 |
| chr13 | 60161535 | 60161585 |
| chr13 | 61001781 | 61001831 |
| chr13 | 61028611 | 61028661 |
| chr13 | 61237284 | 61237334 |
| chr13 | 61489963 | 61490013 |
| chr13 | 61760917 | 61760967 |
| chr13 | 61849698 | 61849748 |
| chr13 | 62675168 | 62675218 |
| chr13 | 63006474 | 63006524 |
| chr13 | 64381530 | 64381580 |
| chr13 | 65366572 | 65366622 |
| chr10 | 42625880 | 42625930 |
| chr13 | 66171172 | 66171222 |
| chr13 | 66224552 | 66224602 |
| chr13 | 67084314 | 67084364 |
| chr13 | 67408757 | 67408807 |
| chr13 | 68851070 | 68851120 |
| chr13 | 69562460 | 69562510 |
| chr13 | 70846875 | 70846925 |
| chr13 | 72546724 | 72546774 |
| chr13 | 73091831 | 73091881 |
| chr13 | 73877253 | 73877303 |
| chr13 | 74026750 | 74026800 |
| chr13 | 75691205 | 75691255 |
| chr13 | 75749980 | 75750030 |
| chr13 | 77526035 | 77526085 |
| chr13 | 77748744 | 77748794 |
| chr13 | 78143115 | 78143165 |
| chr13 | 79571623 | 79571673 |
| chr13 | 81195226 | 81195276 |
| chr13 | 82047321 | 82047371 |
| chr13 | 82161797 | 82161847 |
| chr13 | 82836075 | 82836125 |
| chr13 | 83074527 | 83074577 |
| chr13 | 83175549 | 83175599 |
| chr13 | 83287713 | 83287763 |
| chr13 | 83456097 | 83456147 |
| chr13 | 84421961 | 84422011 |
| chr13 | 84447409 | 84447459 |
| chr13 | 85002098 | 85002148 |
| chr13 | 85474968 | 85475018 |
| chr13 | 85946525 | 85946575 |
| chr13 | 85947674 | 85947724 |
| chr13 | 86205023 | 86205073 |
| chr13 | 86318201 | 86318251 |
| chr13 | 87403628 | 87403678 |
| chr13 | 87678913 | 87678963 |
| chr13 | 88051789 | 88051839 |
| chr13 | 88556100 | 88556150 |
| chr13 | 88963769 | 88963819 |
| chr10 | 43117707 | 43117757 |
| chr13 | 90974787 | 90974837 |
| chr13 | 92981197 | 92981247 |
| chr13 | 92980944 | 92980994 |
| chr13 | 93619085 | 93619135 |
| chr13 | 93624225 | 93624275 |
| chr13 | 93793734 | 93793784 |
| chr13 | 95920161 | 95920211 |
| chr13 | 96404922 | 96404972 |
| chr13 | 96574824 | 96574874 |
| chr13 | 97081481 | 97081531 |
| chr13 | 97506068 | 97506118 |
| chr10 | 43231649 | 43231699 |
| chr13 | 97744893 | 97744943 |
| chr10 | 43231302 | 43231352 |
| chr13 | 98194285 | 98194335 |
| chr13 | 98822595 | 98822645 |
| chr13 | 100942505 | 100942555 |
| chr13 | 102656309 | 102656359 |
| chr10 | 43540235 | 43540285 |
| chr13 | 103856039 | 103856089 |
| chr13 | 103855841 | 103855891 |
| chr13 | 104664019 | 104664069 |
| chr13 | 104977392 | 104977442 |
| chr13 | 105242411 | 105242461 |
| chr13 | 105292923 | 105292973 |
| chr13 | 105292841 | 105292891 |
| chr13 | 105384951 | 105385001 |
| chr13 | 106057552 | 106057602 |
| chr13 | 107034510 | 107034560 |
| chr13 | 107128606 | 107128656 |
| chr10 | 43718493 | 43718543 |
| chr10 | 43718360 | 43718410 |
| chr13 | 107383651 | 107383701 |
| chr13 | 107558217 | 107558267 |
| chr13 | 107654863 | 107654913 |
| chr13 | 107693391 | 107693441 |
| chr13 | 108266842 | 108266892 |
| chr13 | 108422670 | 108422720 |
| chr13 | 108422530 | 108422580 |
| chr13 | 108796648 | 108796698 |
| chr13 | 108918272 | 108918322 |
| chr13 | 109060414 | 109060464 |
| chr13 | 109395606 | 109395656 |
| chr13 | 109433255 | 109433305 |
| chr10 | 44089462 | 44089512 |
| chr13 | 109462661 | 109462711 |
| chr10 | 44089692 | 44089742 |
| chr13 | 109571145 | 109571195 |
| chr13 | 109946413 | 109946463 |
| chr13 | 110504101 | 110504151 |
| chr13 | 110510871 | 110510921 |
| chr13 | 110515386 | 110515436 |
| chr13 | 110546628 | 110546678 |
| chr13 | 110638986 | 110639036 |
| chr13 | 110789808 | 110789858 |
| chr13 | 110835545 | 110835595 |
| chr10 | 44535441 | 44535491 |
| chr13 | 110850055 | 110850105 |
| chr13 | 110862863 | 110862913 |
| chr10 | 44537559 | 44537609 |
| chr13 | 111156377 | 111156427 |
| chr13 | 111156307 | 111156357 |
| chr13 | 111287134 | 111287184 |
| chr13 | 111286933 | 111286983 |
| chr13 | 111374621 | 111374671 |
| chr13 | 111560820 | 111560870 |
| chr13 | 112217111 | 112217161 |
| chr13 | 112500214 | 112500264 |
| chr13 | 112766290 | 112766340 |
| chr13 | 112819446 | 112819496 |
| chr14 | 18331843 | 18331893 |
| chr14 | 18331788 | 18331838 |
| chr14 | 18343219 | 18343269 |
| chr14 | 18382180 | 18382230 |
| chr14 | 18405338 | 18405388 |
| chr14 | 18976343 | 18976393 |
| chr14 | 19013552 | 19013602 |
| chr14 | 19595168 | 19595218 |
| chr14 | 20027533 | 20027583 |
| chr14 | 20441597 | 20441647 |
| chr14 | 21210758 | 21210808 |
| chr14 | 22393930 | 22393980 |
| chr14 | 22393844 | 22393894 |
| chr14 | 23018761 | 23018811 |
| chr14 | 23086795 | 23086845 |
| chr14 | 24989571 | 24989621 |
| chr14 | 25266245 | 25266295 |
| chr14 | 25911841 | 25911891 |
| chr14 | 28160143 | 28160193 |
| chr14 | 28410691 | 28410741 |
| chr14 | 28988650 | 28988700 |
| chr14 | 29927898 | 29927948 |
| chr14 | 32420193 | 32420243 |
| chr14 | 32539370 | 32539420 |
| chr14 | 33286669 | 33286719 |
| chr14 | 33828338 | 33828388 |
| chr14 | 33848720 | 33848770 |
| chr14 | 34361299 | 34361349 |
| chr14 | 36898760 | 36898810 |
| chr10 | 48802804 | 48802854 |
| chr14 | 39928462 | 39928512 |
| chr14 | 40060777 | 40060827 |
| chr14 | 41336008 | 41336058 |
| chr14 | 41519661 | 41519711 |
| chr14 | 41793700 | 41793750 |
| chr14 | 42116706 | 42116756 |
| chr14 | 43539658 | 43539708 |
| chr14 | 43742580 | 43742630 |
| chr14 | 45010989 | 45011039 |
| chr14 | 45011173 | 45011223 |
| chr14 | 45011366 | 45011416 |
| chr14 | 45065795 | 45065845 |
| chr14 | 45209457 | 45209507 |
| chr14 | 45453220 | 45453270 |
| chr14 | 45658056 | 45658106 |
| chr14 | 45695191 | 45695241 |
| chr14 | 47799126 | 47799176 |
| chr14 | 48027224 | 48027274 |
| chr14 | 48564480 | 48564530 |
| chr14 | 48734087 | 48734137 |
| chr14 | 48759687 | 48759737 |
| chr14 | 48872942 | 48872992 |
| chr14 | 48998462 | 48998512 |
| chr14 | 49011794 | 49011844 |
| chr14 | 49417061 | 49417111 |
| chr10 | 49875236 | 49875286 |
| chr14 | 51289156 | 51289206 |
| chr14 | 51358080 | 51358130 |
| chr14 | 52851814 | 52851864 |
| chr14 | 53546416 | 53546466 |
| chr14 | 53566891 | 53566941 |
| chr14 | 53935954 | 53936004 |
| chr14 | 54021236 | 54021286 |
| chr14 | 54171872 | 54171922 |
| chr10 | 50093323 | 50093373 |
| chr14 | 55134116 | 55134166 |
| chr14 | 55327233 | 55327283 |
| chr14 | 55602627 | 55602677 |
| chr14 | 56057064 | 56057114 |
| chr14 | 57237720 | 57237770 |
| chr14 | 57282083 | 57282133 |
| chr14 | 57623612 | 57623662 |
| chr14 | 58999891 | 58999941 |
| chr14 | 59622519 | 59622569 |
| chr14 | 60071550 | 60071600 |
| chr14 | 60513579 | 60513629 |
| chr14 | 61365643 | 61365693 |
| chr10 | 50860986 | 50861036 |
| chr14 | 62168059 | 62168109 |
| chr10 | 50861340 | 50861390 |
| chr14 | 62341801 | 62341851 |
| chr14 | 63572535 | 63572585 |
| chr14 | 66244791 | 66244841 |
| chr14 | 66245243 | 66245293 |
| chr14 | 66335889 | 66335939 |
| chr14 | 67312456 | 67312506 |
| chr14 | 68991832 | 68991882 |
| chr14 | 68991716 | 68991766 |
| chr14 | 69588903 | 69588953 |
| chr14 | 69625718 | 69625768 |
| chr14 | 69753404 | 69753454 |
| chr14 | 69771401 | 69771451 |
| chr14 | 69842721 | 69842771 |
| chr14 | 69917591 | 69917641 |
| chr14 | 69921435 | 69921485 |
| chr14 | 69970655 | 69970705 |
| chr14 | 72066958 | 72067008 |
| chr10 | 52058632 | 52058682 |
| chr14 | 72323518 | 72323568 |
| chr14 | 73030697 | 73030747 |
| chr10 | 52511442 | 52511492 |
| chr10 | 1932497 | 1932547 |
| chr14 | 74688874 | 74688924 |
| chr14 | 75775508 | 75775558 |
| chr14 | 75880879 | 75880929 |
| chr14 | 76152163 | 76152213 |
| chr14 | 76223681 | 76223731 |
| chr10 | 1949067 | 1949117 |
| chr14 | 76569795 | 76569845 |
| chr14 | 77336671 | 77336721 |
| chr14 | 78860594 | 78860644 |
| chr14 | 79211124 | 79211174 |
| chr14 | 79432601 | 79432651 |
| chr14 | 79446987 | 79447037 |
| chr14 | 79478772 | 79478822 |
| chr14 | 80803824 | 80803874 |
| chr14 | 81120912 | 81120962 |
| chr10 | 53882990 | 53883040 |
| chr14 | 81560030 | 81560080 |
| chr14 | 81884709 | 81884759 |
| chr14 | 82004118 | 82004168 |
| chr14 | 82662542 | 82662592 |
| chr14 | 82895105 | 82895155 |
| chr14 | 83494539 | 83494589 |
| chr14 | 84276655 | 84276705 |
| chr14 | 85015790 | 85015840 |
| chr10 | 54123305 | 54123355 |
| chr14 | 85587054 | 85587104 |
| chr14 | 87237244 | 87237294 |
| chr14 | 88066271 | 88066321 |
| chr14 | 88125836 | 88125886 |
| chr14 | 88283794 | 88283844 |
| chr14 | 88342508 | 88342558 |
| chr14 | 89714516 | 89714566 |
| chr14 | 89992937 | 89992987 |
| chr10 | 55050860 | 55050910 |
| chr10 | 55208723 | 55208773 |
| chr14 | 90711747 | 90711797 |
| chr14 | 91150406 | 91150456 |
| chr14 | 91359203 | 91359253 |
| chr14 | 91376114 | 91376164 |
| chr14 | 91474404 | 91474454 |
| chr14 | 91679165 | 91679215 |
| chr14 | 91796268 | 91796318 |
| chr14 | 92249703 | 92249753 |
| chr14 | 92327056 | 92327106 |
| chr14 | 92345474 | 92345524 |
| chr14 | 92622554 | 92622604 |
| chr14 | 93190033 | 93190083 |
| chr14 | 93437594 | 93437644 |
| chr14 | 93626408 | 93626458 |
| chr14 | 94011841 | 94011891 |
| chr14 | 94030877 | 94030927 |
| chr14 | 94637515 | 94637565 |
| chr14 | 95095712 | 95095762 |
| chr14 | 98100114 | 98100164 |
| chr14 | 98510862 | 98510912 |
| chr14 | 98621978 | 98622028 |
| chr14 | 98627075 | 98627125 |
| chr10 | 57552657 | 57552707 |
| chr14 | 99129424 | 99129474 |
| chr14 | 99200353 | 99200403 |
| chr14 | 99207767 | 99207817 |
| chr14 | 99228630 | 99228680 |
| chr14 | 99416683 | 99416733 |
| chr14 | 99490230 | 99490280 |
| chr10 | 57963877 | 57963927 |
| chr14 | 99633786 | 99633836 |
| chr10 | 58073242 | 58073292 |
| chr14 | 99966539 | 99966589 |
| chr14 | 100047250 | 100047300 |
| chr14 | 100047165 | 100047215 |
| chr14 | 100048830 | 100048880 |
| chr14 | 100178820 | 100178870 |
| chr14 | 100353991 | 100354041 |
| chr14 | 100389464 | 100389514 |
| chr14 | 100529975 | 100530025 |
| chr14 | 101057480 | 101057530 |
| chr14 | 101106260 | 101106310 |
| chr14 | 101217495 | 101217545 |
| chr10 | 59181833 | 59181883 |
| chr14 | 101472452 | 101472502 |
| chr14 | 101525340 | 101525390 |
| chr14 | 101717064 | 101717114 |
| chr14 | 101738835 | 101738885 |
| chr14 | 101768380 | 101768430 |
| chr14 | 101810089 | 101810139 |
| chr14 | 102204340 | 102204390 |
| chr14 | 102313111 | 102313161 |
| chr14 | 102312936 | 102312986 |
| chr14 | 102391141 | 102391191 |
| chr14 | 102509387 | 102509437 |
| chr14 | 102525042 | 102525092 |
| chr14 | 102527138 | 102527188 |
| chr14 | 102668279 | 102668329 |
| chr14 | 102750844 | 102750894 |
| chr10 | 59721874 | 59721924 |
| chr10 | 59722937 | 59722987 |
| chr14 | 102957354 | 102957404 |
| chr14 | 103062969 | 103063019 |
| chr14 | 103066584 | 103066634 |
| chr14 | 103100312 | 103100362 |
| chr14 | 103240034 | 103240084 |
| chr14 | 103260970 | 103261020 |
| chr10 | 60029085 | 60029135 |
| chr10 | 60226806 | 60226856 |
| chr14 | 103554769 | 103554819 |
| chr10 | 60259491 | 60259541 |
| chr14 | 103871137 | 103871187 |
| chr14 | 104786663 | 104786713 |
| chr14 | 104932537 | 104932587 |
| chr14 | 104963949 | 104963999 |
| chr15 | 18793313 | 18793363 |
| chr15 | 18834590 | 18834640 |
| chr10 | 60886548 | 60886598 |
| chr15 | 19941143 | 19941193 |
| chr15 | 20512941 | 20512991 |
| chr15 | 21261738 | 21261788 |
| chr15 | 21315109 | 21315159 |
| chr15 | 21333591 | 21333641 |
| chr15 | 21337938 | 21337988 |
| chr10 | 61037056 | 61037106 |
| chr10 | 61037300 | 61037350 |
| chr15 | 21542576 | 21542626 |
| chr15 | 21721523 | 21721573 |
| chr15 | 21774081 | 21774131 |
| chr15 | 22095918 | 22095968 |
| chr15 | 22432333 | 22432383 |
| chr15 | 22631056 | 22631106 |
| chr15 | 22839882 | 22839932 |
| chr15 | 23816087 | 23816137 |
| chr15 | 24614617 | 24614667 |
| chr10 | 61467438 | 61467488 |
| chr15 | 25477788 | 25477838 |
| chr15 | 26142158 | 26142208 |
| chr15 | 26163141 | 26163191 |
| chr15 | 27033848 | 27033898 |
| chr15 | 27038405 | 27038455 |
| chr15 | 27042279 | 27042329 |
| chr15 | 27993763 | 27993813 |
| chr15 | 28779327 | 28779377 |
| chr15 | 28877693 | 28877743 |
| chr10 | 62347092 | 62347142 |
| chr15 | 29249491 | 29249541 |
| chr15 | 29265912 | 29265962 |
| chr15 | 29457989 | 29458039 |
| chr15 | 29642050 | 29642100 |
| chr15 | 30161327 | 30161377 |
| chr15 | 31169853 | 31169903 |
| chr10 | 62763732 | 62763782 |
| chr10 | 62825576 | 62825626 |
| chr15 | 32033447 | 32033497 |
| chr10 | 62960391 | 62960441 |
| chr15 | 32471919 | 32471969 |
| chr15 | 32696064 | 32696114 |
| chr15 | 33008859 | 33008909 |
| chr15 | 33079590 | 33079640 |
| chr10 | 63035118 | 63035168 |
| chr15 | 34073076 | 34073126 |
| chr15 | 34479678 | 34479728 |
| chr15 | 36016013 | 36016063 |
| chr15 | 36556125 | 36556175 |
| chr15 | 36555988 | 36556038 |
| chr15 | 36790865 | 36790915 |
| chr15 | 37458973 | 37459023 |
| chr15 | 37552962 | 37553012 |
| chr15 | 37820735 | 37820785 |
| chr15 | 38576589 | 38576639 |
| chr15 | 38814309 | 38814359 |
| chr15 | 40000706 | 40000756 |
| chr15 | 40799646 | 40799696 |
| chr10 | 64234447 | 64234497 |
| chr15 | 41946204 | 41946254 |
| chr15 | 42291112 | 42291162 |
| chr15 | 42808318 | 42808368 |
| chr15 | 42942890 | 42942940 |
| chr15 | 43621850 | 43621900 |
| chr15 | 43625931 | 43625981 |
| chr15 | 43628251 | 43628301 |
| chr15 | 43630901 | 43630951 |
| chr15 | 43777961 | 43778011 |
| chr15 | 43795697 | 43795747 |
| chr15 | 43973399 | 43973449 |
| chr15 | 49609220 | 49609270 |
| chr15 | 50108731 | 50108781 |
| chr15 | 50140667 | 50140717 |
| chr15 | 50140591 | 50140641 |
| chr15 | 50545311 | 50545361 |
| chr15 | 51492229 | 51492279 |
| chr15 | 51705839 | 51705889 |
| chr15 | 51782046 | 51782096 |
| chr15 | 51919168 | 51919218 |
| chr15 | 51918908 | 51918958 |
| chr15 | 52102159 | 52102209 |
| chr15 | 52102068 | 52102118 |
| chr15 | 52155315 | 52155365 |
| chr15 | 53099777 | 53099827 |
| chr10 | 64890222 | 64890272 |
| chr15 | 53758664 | 53758714 |
| chr15 | 53798580 | 53798630 |
| chr15 | 54460645 | 54460695 |
| chr15 | 54823714 | 54823764 |
| chr15 | 54904827 | 54904877 |
| chr15 | 55739245 | 55739295 |
| chr15 | 56122109 | 56122159 |
| chr15 | 56256921 | 56256971 |
| chr15 | 57258578 | 57258628 |
| chr15 | 57539031 | 57539081 |
| chr15 | 57744246 | 57744296 |
| chr10 | 65702323 | 65702373 |
| chr15 | 59791893 | 59791943 |
| chr15 | 60113665 | 60113715 |
| chr15 | 60331970 | 60332020 |
| chr15 | 60389451 | 60389501 |
| chr15 | 61170987 | 61171037 |
| chr15 | 61459240 | 61459290 |
| chr10 | 66659968 | 66660018 |
| chr10 | 66866256 | 66866306 |
| chr15 | 62393346 | 62393396 |
| chr15 | 63527843 | 63527893 |
| chr15 | 63529569 | 63529619 |
| chr15 | 63780368 | 63780418 |
| chr15 | 63780189 | 63780239 |
| chr15 | 64121705 | 64121755 |
| chr15 | 64131222 | 64131272 |
| chr15 | 64180236 | 64180286 |
| chr15 | 64248573 | 64248623 |
| chr15 | 64610784 | 64610834 |
| chr15 | 64984973 | 64985023 |
| chr15 | 65997074 | 65997124 |
| chr15 | 66775281 | 66775331 |
| chr15 | 66852136 | 66852186 |
| chr15 | 66888512 | 66888562 |
| chr15 | 67319348 | 67319398 |
| chr15 | 67358279 | 67358329 |
| chr15 | 67521201 | 67521251 |
| chr15 | 67654822 | 67654872 |
| chr15 | 68188877 | 68188927 |
| chr15 | 68279076 | 68279126 |
| chr15 | 68487959 | 68488009 |
| chr15 | 68588526 | 68588576 |
| chr15 | 68794058 | 68794108 |
| chr10 | 68904275 | 68904325 |
| chr15 | 70711252 | 70711302 |
| chr10 | 68904619 | 68904669 |
| chr10 | 68904940 | 68904990 |
| chr15 | 71182443 | 71182493 |
| chr15 | 71431457 | 71431507 |
| chr15 | 72503511 | 72503561 |
| chr15 | 72508130 | 72508180 |
| chr15 | 72747760 | 72747810 |
| chr15 | 72988902 | 72988952 |
| chr15 | 73164521 | 73164571 |
| chr15 | 74920261 | 74920311 |
| chr15 | 74923232 | 74923282 |
| chr15 | 74924798 | 74924848 |
| chr15 | 75051622 | 75051672 |
| chr15 | 75083503 | 75083553 |
| chr15 | 76356462 | 76356512 |
| chr15 | 76411484 | 76411534 |
| chr15 | 76713900 | 76713950 |
| chr15 | 76913998 | 76914048 |
| chr15 | 77699950 | 77700000 |
| chr15 | 78247288 | 78247338 |
| chr15 | 79444909 | 79444959 |
| chr15 | 79601814 | 79601864 |
| chr15 | 81185080 | 81185130 |
| chr15 | 81714328 | 81714378 |
| chr15 | 82975774 | 82975824 |
| chr15 | 83011349 | 83011399 |
| chr15 | 83376282 | 83376332 |
| chr15 | 84034495 | 84034545 |
| chr15 | 84284241 | 84284291 |
| chr15 | 85721108 | 85721158 |
| chr15 | 86677848 | 86677898 |
| chr15 | 86703659 | 86703709 |
| chr15 | 86709743 | 86709793 |
| chr15 | 87273698 | 87273748 |
| chr15 | 88193687 | 88193737 |
| chr15 | 88783276 | 88783326 |
| chr15 | 88932073 | 88932123 |
| chr15 | 88969803 | 88969853 |
| chr15 | 90416999 | 90417049 |
| chr15 | 90551776 | 90551826 |
| chr15 | 91370024 | 91370074 |
| chr15 | 91737448 | 91737498 |
| chr15 | 92182700 | 92182750 |
| chr15 | 92194416 | 92194466 |
| chr15 | 92306831 | 92306881 |
| chr15 | 92849863 | 92849913 |
| chr15 | 93353352 | 93353402 |
| chr15 | 94022203 | 94022253 |
| chr15 | 94336774 | 94336824 |
| chr15 | 95099680 | 95099730 |
| chr15 | 95795651 | 95795701 |
| chr15 | 96500530 | 96500580 |
| chr15 | 96696009 | 96696059 |
| chr15 | 96766828 | 96766878 |
| chr10 | 69957663 | 69957713 |
| chr10 | 69957940 | 69957990 |
| chr15 | 98241534 | 98241584 |
| chr15 | 98571503 | 98571553 |
| chr15 | 99542284 | 99542334 |
| chr15 | 99683550 | 99683600 |
| chr15 | 99750086 | 99750136 |
| chr10 | 70147677 | 70147727 |
| chr16 | 484716 | 484766 |
| chr10 | 70213357 | 70213407 |
| chr10 | 70217354 | 70217404 |
| chr16 | 726160 | 726210 |
| chr16 | 751222 | 751272 |
| chr16 | 751040 | 751090 |
| chr16 | 750937 | 750987 |
| chr16 | 799691 | 799741 |
| chr16 | 896450 | 896500 |
| chr16 | 1894057 | 1894107 |
| chr16 | 1925473 | 1925523 |
| chr16 | 2196567 | 2196617 |
| chr16 | 2786249 | 2786299 |
| chr16 | 2993567 | 2993617 |
| chr16 | 3093650 | 3093700 |
| chr16 | 3180386 | 3180436 |
| chr16 | 3205198 | 3205248 |
| chr16 | 3223948 | 3223998 |
| chr16 | 3248889 | 3248939 |
| chr10 | 3015419 | 3015469 |
| chr16 | 3560809 | 3560859 |
| chr10 | 3017138 | 3017188 |
| chr16 | 3940651 | 3940701 |
| chr10 | 3024852 | 3024902 |
| chr16 | 5143793 | 5143843 |
| chr16 | 5179911 | 5179961 |
| chr16 | 5529636 | 5529686 |
| chr16 | 6049830 | 6049880 |
| chr16 | 6994071 | 6994121 |
| chr16 | 10279716 | 10279766 |
| chr16 | 10300759 | 10300809 |
| chr16 | 10339118 | 10339168 |
| chr16 | 10437615 | 10437665 |
| chr16 | 11247497 | 11247547 |
| chr16 | 11531018 | 11531068 |
| chr16 | 11596241 | 11596291 |
| chr16 | 11697535 | 11697585 |
| chr16 | 12017706 | 12017756 |
| chr16 | 12114668 | 12114718 |
| chr16 | 12221655 | 12221705 |
| chr16 | 12221468 | 12221518 |
| chr16 | 12222473 | 12222523 |
| chr16 | 12862584 | 12862634 |
| chr16 | 13494592 | 13494642 |
| chr16 | 14285640 | 14285690 |
| chr16 | 16906758 | 16906808 |
| chr16 | 18032885 | 18032935 |
| chr16 | 18044193 | 18044243 |
| chr16 | 18101507 | 18101557 |
| chr16 | 18259831 | 18259881 |
| chr16 | 19166714 | 19166764 |
| chr16 | 19257212 | 19257262 |
| chr16 | 19258937 | 19258987 |
| chr16 | 19805588 | 19805638 |
| chr16 | 19969528 | 19969578 |
| chr16 | 20241212 | 20241262 |
| chr16 | 20720540 | 20720590 |
| chr16 | 20721230 | 20721280 |
| chr16 | 20900756 | 20900806 |
| chr10 | 71757549 | 71757599 |
| chr16 | 22025403 | 22025453 |
| chr16 | 23652259 | 23652309 |
| chr16 | 23652053 | 23652103 |
| chr16 | 23721734 | 23721784 |
| chr16 | 23980598 | 23980648 |
| chr16 | 24017450 | 24017500 |
| chr16 | 24492894 | 24492944 |
| chr16 | 24640971 | 24641021 |
| chr16 | 25043347 | 25043397 |
| chr16 | 25044777 | 25044827 |
| chr16 | 25959232 | 25959282 |
| chr16 | 26242030 | 26242080 |
| chr16 | 26392043 | 26392093 |
| chr16 | 27489357 | 27489407 |
| chr16 | 27953015 | 27953065 |
| chr16 | 28013983 | 28014033 |
| chr16 | 28860446 | 28860496 |
| chr16 | 29044602 | 29044652 |
| chr16 | 29249802 | 29249852 |
| chr16 | 29689378 | 29689428 |
| chr16 | 29711162 | 29711212 |
| chr10 | 72609960 | 72610010 |
| chr16 | 30645480 | 30645530 |
| chr16 | 31161506 | 31161556 |
| chr16 | 31316426 | 31316476 |
| chr16 | 32431207 | 32431257 |
| chr16 | 33088230 | 33088280 |
| chr16 | 35547534 | 35547584 |
| chr16 | 47340977 | 47341027 |
| chr16 | 47904620 | 47904670 |
| chr16 | 48510154 | 48510204 |
| chr16 | 48916472 | 48916522 |
| chr16 | 49584112 | 49584162 |
| chr16 | 49709413 | 49709463 |
| chr16 | 50303180 | 50303230 |
| chr16 | 50314824 | 50314874 |
| chr16 | 50314726 | 50314776 |
| chr16 | 50630494 | 50630544 |
| chr16 | 51131489 | 51131539 |
| chr10 | 73026693 | 73026743 |
| chr10 | 73029971 | 73030021 |
| chr16 | 52094190 | 52094240 |
| chr16 | 52458502 | 52458552 |
| chr16 | 52627888 | 52627938 |
| chr16 | 53693173 | 53693223 |
| chr16 | 54246446 | 54246496 |
| chr10 | 73141713 | 73141763 |
| chr16 | 54982509 | 54982559 |
| chr10 | 73143055 | 73143105 |
| chr16 | 56425008 | 56425058 |
| chr16 | 56517372 | 56517422 |
| chr16 | 56641272 | 56641322 |
| chr16 | 56641035 | 56641085 |
| chr16 | 56812768 | 56812818 |
| chr16 | 57102137 | 57102187 |
| chr16 | 57302318 | 57302368 |
| chr16 | 57892858 | 57892908 |
| chr16 | 58045928 | 58045978 |
| chr16 | 58064548 | 58064598 |
| chr16 | 58827236 | 58827286 |
| chr16 | 59177054 | 59177104 |
| chr16 | 59358523 | 59358573 |
| chr16 | 60128242 | 60128292 |
| chr16 | 60377051 | 60377101 |
| chr16 | 60583346 | 60583396 |
| chr16 | 61276410 | 61276460 |
| chr16 | 62308324 | 62308374 |
| chr16 | 63476379 | 63476429 |
| chr16 | 63735085 | 63735135 |
| chr16 | 64233038 | 64233088 |
| chr16 | 65166885 | 65166935 |
| chr16 | 65774678 | 65774728 |
| chr16 | 66771987 | 66772037 |
| chr10 | 73715294 | 73715344 |
| chr16 | 68066143 | 68066193 |
| chr16 | 68134896 | 68134946 |
| chr16 | 68134692 | 68134742 |
| chr16 | 68431780 | 68431830 |
| chr16 | 68519867 | 68519917 |
| chr10 | 74162528 | 74162578 |
| chr10 | 74162678 | 74162728 |
| chr16 | 71960695 | 71960745 |
| chr16 | 72229923 | 72229973 |
| chr16 | 73715126 | 73715176 |
| chr16 | 73831487 | 73831537 |
| chr16 | 74056664 | 74056714 |
| chr16 | 74373896 | 74373946 |
| chr16 | 74424697 | 74424747 |
| chr16 | 74427189 | 74427239 |
| chr16 | 74428599 | 74428649 |
| chr16 | 75011723 | 75011773 |
| chr16 | 75206915 | 75206965 |
| chr16 | 75369771 | 75369821 |
| chr16 | 75370295 | 75370345 |
| chr16 | 75406922 | 75406972 |
| chr16 | 76606585 | 76606635 |
| chr16 | 77821794 | 77821844 |
| chr16 | 78365918 | 78365968 |
| chr16 | 78366034 | 78366084 |
| chr16 | 78584074 | 78584124 |
| chr16 | 80962889 | 80962939 |
| chr16 | 81017100 | 81017150 |
| chr16 | 81037517 | 81037567 |
| chr16 | 81100633 | 81100683 |
| chr16 | 81100461 | 81100511 |
| chr16 | 81182986 | 81183036 |
| chr16 | 81514982 | 81515032 |
| chr16 | 81527362 | 81527412 |
| chr16 | 81573361 | 81573411 |
| chr10 | 74733149 | 74733199 |
| chr10 | 74734188 | 74734238 |
| chr10 | 74734335 | 74734385 |
| chr16 | 82107637 | 82107687 |
| chr16 | 83888882 | 83888932 |
| chr16 | 83888801 | 83888851 |
| chr16 | 84011623 | 84011673 |
| chr16 | 84011832 | 84011882 |
| chr10 | 3305135 | 3305185 |
| chr16 | 84280691 | 84280741 |
| chr16 | 84280607 | 84280657 |
| chr16 | 84298435 | 84298485 |
| chr16 | 84332810 | 84332860 |
| chr16 | 84338656 | 84338706 |
| chr10 | 3304915 | 3304965 |
| chr16 | 84447812 | 84447862 |
| chr16 | 84481354 | 84481404 |
| chr16 | 84489047 | 84489097 |
| chr16 | 84491048 | 84491098 |
| chr16 | 84728664 | 84728714 |
| chr16 | 84811886 | 84811936 |
| chr16 | 85084022 | 85084072 |
| chr16 | 85512859 | 85512909 |
| chr16 | 85629175 | 85629225 |
| chr16 | 85707442 | 85707492 |
| chr16 | 85827346 | 85827396 |
| chr16 | 85848601 | 85848651 |
| chr16 | 86003288 | 86003338 |
| chr10 | 74995980 | 74996030 |
| chr16 | 86297677 | 86297727 |
| chr16 | 86509546 | 86509596 |
| chr16 | 86510181 | 86510231 |
| chr16 | 86590171 | 86590221 |
| chr16 | 86744999 | 86745049 |
| chr16 | 86960616 | 86960666 |
| chr16 | 87357200 | 87357250 |
| chr16 | 87471895 | 87471945 |
| chr16 | 87615363 | 87615413 |
| chr16 | 87982180 | 87982230 |
| chr16 | 88203517 | 88203567 |
| chr16 | 88501752 | 88501802 |
| chr16 | 88501610 | 88501660 |
| chr16 | 88565596 | 88565646 |
| chr16 | 88576639 | 88576689 |
| chr10 | 75337469 | 75337519 |
| chr16 | 88661183 | 88661233 |
| chr10 | 75378179 | 75378229 |
| chr16 | 88816640 | 88816690 |
| chr16 | 88831924 | 88831974 |
| chr16 | 89029919 | 89029969 |
| chr16 | 89108789 | 89108839 |
| chr16 | 89134294 | 89134344 |
| chr16 | 89157226 | 89157276 |
| chr17 | 150299 | 150349 |
| chr17 | 279849 | 279899 |
| chr17 | 302959 | 303009 |
| chr17 | 326904 | 326954 |
| chr17 | 449625 | 449675 |
| chr10 | 76048646 | 76048696 |
| chr17 | 876295 | 876345 |
| chr17 | 946990 | 947040 |
| chr17 | 1928094 | 1928144 |
| chr10 | 76264354 | 76264404 |
| chr17 | 2726633 | 2726683 |
| chr10 | 76471623 | 76471673 |
| chr10 | 76477370 | 76477420 |
| chr17 | 4230259 | 4230309 |
| chr17 | 4514093 | 4514143 |
| chr17 | 4707966 | 4708016 |
| chr17 | 4759712 | 4759762 |
| chr17 | 5208523 | 5208573 |
| chr17 | 5288332 | 5288382 |
| chr17 | 5322864 | 5322914 |
| chr17 | 5393709 | 5393759 |
| chr17 | 5894374 | 5894424 |
| chr17 | 5940066 | 5940116 |
| chr17 | 6033862 | 6033912 |
| chr17 | 6500221 | 6500271 |
| chr17 | 6976142 | 6976192 |
| chr17 | 7185542 | 7185592 |
| chr17 | 7198008 | 7198058 |
| chr17 | 7252371 | 7252421 |
| chr17 | 7264492 | 7264542 |
| chr17 | 8047310 | 8047360 |
| chr17 | 8157601 | 8157651 |
| chr17 | 8967704 | 8967754 |
| chr17 | 9430508 | 9430558 |
| chr17 | 9563207 | 9563257 |
| chr17 | 9597522 | 9597572 |
| chr17 | 9837645 | 9837695 |
| chr17 | 10089313 | 10089363 |
| chr17 | 10189212 | 10189262 |
| chr17 | 10368507 | 10368557 |
| chr17 | 11166569 | 11166619 |
| chr17 | 11218555 | 11218605 |
| chr17 | 11222994 | 11223044 |
| chr17 | 11711370 | 11711420 |
| chr17 | 12456603 | 12456653 |
| chr17 | 12501162 | 12501212 |
| chr17 | 12533832 | 12533882 |
| chr17 | 12963691 | 12963741 |
| chr17 | 13375774 | 13375824 |
| chr17 | 13923277 | 13923327 |
| chr17 | 14473415 | 14473465 |
| chr17 | 14626539 | 14626589 |
| chr17 | 14875668 | 14875718 |
| chr17 | 14902006 | 14902056 |
| chr17 | 14938767 | 14938817 |
| chr17 | 14939913 | 14939963 |
| chr17 | 15114820 | 15114870 |
| chr17 | 15121388 | 15121438 |
| chr17 | 15582313 | 15582363 |
| chr17 | 15582374 | 15582424 |
| chr17 | 15619490 | 15619540 |
| chr17 | 15619291 | 15619341 |
| chr10 | 79321637 | 79321687 |
| chr10 | 79412445 | 79412495 |
| chr17 | 18921594 | 18921644 |
| chr17 | 19178060 | 19178110 |
| chr17 | 19551164 | 19551214 |
| chr17 | 20050062 | 20050112 |
| chr17 | 20052359 | 20052409 |
| chr17 | 20061485 | 20061535 |
| chr17 | 21122596 | 21122646 |
| chr17 | 21151191 | 21151241 |
| chr17 | 21179866 | 21179916 |
| chr17 | 21189114 | 21189164 |
| chr17 | 25748194 | 25748244 |
| chr17 | 26390770 | 26390820 |
| chr17 | 26902719 | 26902769 |
| chr17 | 26927387 | 26927437 |
| chr17 | 26929269 | 26929319 |
| chr17 | 27276932 | 27276982 |
| chr17 | 28116413 | 28116463 |
| chr17 | 28244775 | 28244825 |
| chr17 | 28521726 | 28521776 |
| chr17 | 28807997 | 28808047 |
| chr17 | 28807937 | 28807987 |
| chr10 | 3737554 | 3737604 |
| chr17 | 29059554 | 29059604 |
| chr17 | 29253046 | 29253096 |
| chr17 | 29586799 | 29586849 |
| chr17 | 30329244 | 30329294 |
| chr17 | 31443010 | 31443060 |
| chr17 | 31442768 | 31442818 |
| chr17 | 32771119 | 32771169 |
| chr17 | 33207952 | 33208002 |
| chr17 | 33244388 | 33244438 |
| chr17 | 33880226 | 33880276 |
| chr17 | 34232256 | 34232306 |
| chr17 | 34367305 | 34367355 |
| chr17 | 34473422 | 34473472 |
| chr17 | 34473309 | 34473359 |
| chr17 | 34615934 | 34615984 |
| chr17 | 36396350 | 36396400 |

Table 15J. Repeat Category ERVL-B4

| | | |
|---|---|---|
| chr17 | 79663446 | 79663496 |
| chr17 | 81655590 | 81655640 |
| chr17 | 81656186 | 81656236 |
| chr10 | 93836012 | 93836062 |
| chr18 | 11714572 | 11714622 |
| chr18 | 50286093 | 50286143 |
| chr19 | 9703332 | 9703382 |
| chr19 | 19734668 | 19734718 |
| chr19 | 19735950 | 19736000 |
| chr19 | 38367114 | 38367164 |
| chr19 | 48561987 | 48562037 |
| chr10 | 107159974 | 107160024 |
| chr19 | 49213673 | 49213723 |
| chr19 | 49306890 | 49306940 |
| chr19 | 49307169 | 49307219 |
| chr10 | 113008190 | 113008240 |
| chr19 | 59498707 | 59498757 |
| chr19 | 59499175 | 59499225 |
| chr19 | 59498636 | 59498686 |
| chr19 | 60418163 | 60418213 |
| chr1 | 2703433 | 2703483 |
| chr1 | 21001713 | 21001763 |
| chr1 | 21003911 | 21003961 |
| chr1 | 21005323 | 21005373 |
| chr1 | 32373377 | 32373427 |
| chr1 | 88570535 | 88570585 |
| chr1 | 95190210 | 95190260 |
| chr1 | 95191644 | 95191694 |
| chr1 | 228742319 | 228742369 |
| chr20 | 62233155 | 62233205 |
| chr21 | 27035141 | 27035191 |
| chr21 | 37597268 | 37597318 |
| chr11 | 1056228 | 1056278 |
| chr21 | 46343979 | 46344029 |
| chr22 | 39559437 | 39559487 |
| chr22 | 47417932 | 47417982 |
| chr22 | 49227911 | 49227961 |
| chrX | 63508636 | 63508686 |
| chrX | 86326808 | 86326858 |
| chrX | 87580189 | 87580239 |
| chrX | 92919015 | 92919065 |
| chrX | 129503557 | 129503607 |
| chrX | 129510700 | 129510750 |
| chrX | 133454977 | 133455027 |
| chrX | 133512850 | 133512900 |
| chrX | 133547781 | 133547831 |
| chrX | 133702475 | 133702525 |
| chrY | 14172419 | 14172469 |
| chr2 | 11623095 | 11623145 |
| chr2 | 21558215 | 21558265 |
| chr2 | 62066404 | 62066454 |
| chr2 | 69520571 | 69520621 |
| chr2 | 95464653 | 95464703 |
| chr2 | 95477329 | 95477379 |
| chr2 | 107875411 | 107875461 |
| chr2 | 113474853 | 113474903 |
| chr11 | 32458605 | 32458655 |
| chr2 | 130362224 | 130362274 |
| chr2 | 159005532 | 159005582 |
| chr2 | 242834373 | 242834423 |
| chr3 | 34926802 | 34926852 |
| chr3 | 46283027 | 46283077 |
| chr3 | 82856433 | 82856483 |
| chr3 | 83845963 | 83846013 |
| chr3 | 185581029 | 185581079 |
| chr4 | 6621795 | 6621845 |
| chr4 | 112556663 | 112556713 |
| chr4 | 125498823 | 125498873 |
| chr4 | 167234601 | 167234651 |
| chr4 | 179331312 | 179331362 |
| chr4 | 188128051 | 188128101 |
| chr11 | 69193964 | 69194014 |
| chr5 | 23271333 | 23271383 |
| chr5 | 30253349 | 30253399 |
| chr5 | 49942088 | 49942138 |
| chr5 | 61025428 | 61025478 |
| chr5 | 80392512 | 80392562 |
| chr11 | 71074451 | 71074501 |
| chr11 | 78047790 | 78047840 |
| chr6 | 33695868 | 33695918 |
| chr6 | 66232320 | 66232370 |
| chr6 | 86911446 | 86911496 |
| chr6 | 94803351 | 94803401 |
| chr10 | 15438107 | 15438157 |
| chr6 | 170646790 | 170646840 |
| chr7 | 3626885 | 3626935 |
| chr7 | 22895240 | 22895290 |
| chr7 | 51145436 | 51145486 |
| chr7 | 52684086 | 52684136 |
| chr7 | 95344897 | 95344947 |
| chr7 | 122154571 | 122154621 |
| chr7 | 133619782 | 133619832 |
| chr7 | 134627117 | 134627167 |
| chr7 | 150811812 | 150811862 |
| chr7 | 152190369 | 152190419 |
| chr7 | 156587986 | 156588036 |
| chr11 | 120472979 | 120473029 |
| chr8 | 22916330 | 22916380 |
| chr8 | 23215788 | 23215838 |
| chr8 | 50201883 | 50201933 |
| chr8 | 103140451 | 103140501 |
| chr8 | 123895484 | 123895534 |
| chr8 | 144932498 | 144932548 |
| chr8 | 144933507 | 144933557 |
| chr9 | 29502206 | 29502256 |
| chr9 | 30288992 | 30289042 |
| chr9 | 30290641 | 30290691 |
| chr9 | 90092279 | 90092329 |
| chr9 | 95171099 | 95171149 |
| chr9 | 127906594 | 127906644 |
| chr12 | 7008041 | 7008091 |
| chr12 | 8829758 | 8829808 |
| chr12 | 63239144 | 63239194 |
| chr12 | 89113498 | 89113548 |
| chr12 | 102529369 | 102529419 |
| chr12 | 103070260 | 103070310 |
| chr12 | 131715407 | 131715457 |
| chr13 | 18282043 | 18282093 |
| chr13 | 27177944 | 27177994 |
| chr10 | 37023962 | 37024012 |
| chr10 | 42625880 | 42625930 |
| chr13 | 109395606 | 109395656 |
| chr10 | 52058632 | 52058682 |
| chr10 | 1932497 | 1932547 |
| chr14 | 103554769 | 103554819 |
| chr15 | 28877693 | 28877743 |
| chr16 | 3180386 | 3180436 |
| chr10 | 3015419 | 3015469 |
| chr16 | 35547534 | 35547584 |
| chr16 | 56425008 | 56425058 |
| chr16 | 86003288 | 86003338 |
| chr16 | 88203517 | 88203567 |
| chr17 | 876295 | 876345 |

Table 15K. Repeat Category GSAT

| | | |
|---|---|---|
| chr1 | 140839821 | 140839871 |
| chr1 | 140841172 | 140841222 |
| chr1 | 140842296 | 140842346 |
| chr21 | 14367127 | 14367177 |
| chr22 | 14476328 | 14476378 |
| chr22 | 14476980 | 14477030 |
| chr22 | 14477878 | 14477928 |
| chr22 | 14478232 | 14478282 |
| chrX | 57249652 | 57249702 |
| chrX | 57252055 | 57252105 |
| chrX | 57253098 | 57253148 |
| chrX | 57255768 | 57255818 |
| chrX | 57258572 | 57258622 |
| chr2 | 31403726 | 31403776 |
| chr2 | 132723394 | 132723444 |
| chr3 | 174852694 | 174852744 |
| chr4 | 7062858 | 7062908 |
| chr4 | 49434782 | 49434832 |
| chr4 | 49435470 | 49435520 |
| chr5 | 177837223 | 177837273 |
| chr8 | 43417880 | 43417930 |
| chr8 | 43418739 | 43418789 |
| chr8 | 43420187 | 43420237 |
| chr8 | 43421368 | 43421418 |
| chr8 | 43423341 | 43423391 |
| chr8 | 43436146 | 43436196 |
| chr8 | 46947006 | 46947056 |
| chr8 | 47032918 | 47032968 |
| chr8 | 47039361 | 47039411 |
| chr8 | 47042168 | 47042218 |
| chr8 | 47057029 | 47057079 |
| chr8 | 47063194 | 47063244 |
| chr8 | 47065152 | 47065202 |
| chr8 | 47069102 | 47069152 |
| chr8 | 47070617 | 47070667 |
| chr8 | 47074813 | 47074863 |
| chr8 | 47105806 | 47105856 |
| chr8 | 47110603 | 47110653 |
| chr8 | 47121169 | 47121219 |
| chr8 | 47122261 | 47122311 |
| chr8 | 47126219 | 47126269 |
| chr8 | 47129687 | 47129737 |
| chr8 | 47131011 | 47131061 |
| chr8 | 47136244 | 47136294 |
| chr8 | 47142204 | 47142254 |
| chr8 | 47144188 | 47144238 |
| chr8 | 47145885 | 47145935 |
| chr8 | 47147133 | 47147183 |
| chr8 | 47148295 | 47148345 |
| chr8 | 47157918 | 47157968 |
| chr8 | 47159194 | 47159244 |
| chr8 | 47162521 | 47162571 |
| chr8 | 47163587 | 47163637 |
| chr8 | 47164854 | 47164904 |
| chr8 | 47168722 | 47168772 |
| chr8 | 47171751 | 47171801 |
| chr8 | 47172827 | 47172877 |
| chr8 | 47177504 | 47177554 |
| chr8 | 47178335 | 47178385 |
| chr8 | 47183041 | 47183091 |
| chr8 | 47189273 | 47189323 |
| chr8 | 47189098 | 47189148 |
| chr8 | 47191211 | 47191261 |
| chr8 | 47204227 | 47204277 |
| chr8 | 47307555 | 47307605 |
| chr8 | 47335857 | 47335907 |
| chr8 | 47340466 | 47340516 |
| chr8 | 47344331 | 47344381 |
| chr8 | 47352798 | 47352848 |
| chr8 | 47359859 | 47359909 |
| chr8 | 47361658 | 47361708 |
| chr8 | 47365194 | 47365244 |
| chr8 | 47366498 | 47366548 |
| chr8 | 47375191 | 47375241 |
| chr9 | 43172902 | 43172952 |
| chr9 | 43174537 | 43174587 |
| chr9 | 43176252 | 43176302 |
| chr9 | 63427466 | 63427516 |
| chr9 | 64968296 | 64968346 |
| chr9 | 64971276 | 64971326 |
| chr12 | 34255417 | 34255467 |
| chr12 | 34333749 | 34333799 |
| chr12 | 34335653 | 34335703 |
| chr12 | 34337071 | 34337121 |
| chr12 | 34340101 | 34340151 |
| chr12 | 34344148 | 34344198 |
| chr12 | 34346821 | 34346871 |
| chr12 | 34348414 | 34348464 |
| chr12 | 34349452 | 34349502 |
| chr12 | 34351406 | 34351456 |
| chr12 | 34355131 | 34355181 |
| chr12 | 34356000 | 34356050 |
| chr12 | 34357433 | 34357483 |
| chr12 | 34361478 | 34361528 |
| chr12 | 34365891 | 34365941 |
| chr12 | 34368023 | 34368073 |
| chr12 | 34370145 | 34370195 |
| chr12 | 34371879 | 34371929 |
| chr12 | 34373142 | 34373192 |
| chr12 | 34375854 | 34375904 |
| chr12 | 34376598 | 34376648 |
| chr12 | 34381192 | 34381242 |
| chr12 | 34384507 | 34384557 |
| chr12 | 34386405 | 34386455 |
| chr12 | 34388750 | 34388800 |
| chr12 | 34391121 | 34391171 |
| chr12 | 34397175 | 34397225 |
| chr12 | 34398620 | 34398670 |
| chr12 | 34400674 | 34400724 |
| chr12 | 34402377 | 34402427 |
| chr12 | 34403978 | 34404028 |
| chr12 | 34407067 | 34407117 |
| chr12 | 34410197 | 34410247 |
| chr12 | 34411525 | 34411575 |
| chr12 | 34413190 | 34413240 |
| chr12 | 34415055 | 34415105 |
| chr12 | 34415954 | 34416004 |
| chr12 | 34418034 | 34418084 |
| chr12 | 34420630 | 34420680 |
| chr12 | 34421456 | 34421506 |
| chr12 | 34422708 | 34422758 |
| chr12 | 34423824 | 34423874 |
| chr12 | 34425483 | 34425533 |
| chr12 | 34427806 | 34427856 |
| chr12 | 34429072 | 34429122 |
| chr12 | 34436147 | 34436197 |
| chr12 | 34437394 | 34437444 |
| chr12 | 34442947 | 34442997 |
| chr12 | 34446019 | 34446069 |
| chr12 | 34447767 | 34447817 |
| chr12 | 34642395 | 34642445 |
| chr12 | 34644020 | 34644070 |
| chr12 | 34645336 | 34645386 |
| chr12 | 34646060 | 34646110 |
| chr12 | 34648711 | 34648761 |
| chr12 | 34649045 | 34649095 |
| chr12 | 34650600 | 34650650 |
| chr12 | 36832980 | 36833030 |
| chr12 | 36835270 | 36835320 |
| chr12 | 36837781 | 36837831 |
| chr12 | 36838599 | 36838649 |
| chr12 | 36840393 | 36840443 |
| chr13 | 17317122 | 17317172 |
| chr17 | 3875990 | 3876040 |

Table 15L. Repeat Category GSATII

| | | |
|---|---|---|
| chr1 | 140839821 | 140839871 |
| chr1 | 140841172 | 140841222 |
| chr1 | 140842296 | 140842346 |
| chr22 | 14476328 | 14476378 |
| chr22 | 14476980 | 14477030 |
| chr22 | 14477878 | 14477928 |
| chr22 | 14478232 | 14478282 |
| chr2 | 132723394 | 132723444 |
| chr4 | 49434782 | 49434832 |
| chr4 | 49435470 | 49435520 |
| chr9 | 43172902 | 43172952 |
| chr9 | 43174537 | 43174587 |
| chr9 | 43176252 | 43176302 |
| chr9 | 63427466 | 63427516 |
| chr9 | 64968296 | 64968346 |
| chr9 | 64971276 | 64971326 |
| chr12 | 34255417 | 34255467 |
| chr12 | 34333749 | 34333799 |
| chr12 | 34335653 | 34335703 |
| chr12 | 34337071 | 34337121 |
| chr12 | 34340101 | 34340151 |
| chr12 | 34344148 | 34344198 |
| chr12 | 34346821 | 34346871 |
| chr12 | 34348414 | 34348464 |
| chr12 | 34349452 | 34349502 |
| chr12 | 34351406 | 34351456 |
| chr12 | 34355131 | 34355181 |
| chr12 | 34356000 | 34356050 |
| chr12 | 34357433 | 34357483 |
| chr12 | 34361478 | 34361528 |
| chr12 | 34365891 | 34365941 |
| chr12 | 34368023 | 34368073 |
| chr12 | 34370145 | 34370195 |
| chr12 | 34371879 | 34371929 |
| chr12 | 34373142 | 34373192 |
| chr12 | 34375854 | 34375904 |
| chr12 | 34376598 | 34376648 |
| chr12 | 34381192 | 34381242 |
| chr12 | 34384507 | 34384557 |
| chr12 | 34386405 | 34386455 |
| chr12 | 34388750 | 34388800 |
| chr12 | 34391121 | 34391171 |
| chr12 | 34397175 | 34397225 |
| chr12 | 34398620 | 34398670 |
| chr12 | 34400674 | 34400724 |
| chr12 | 34402377 | 34402427 |
| chr12 | 34403978 | 34404028 |
| chr12 | 34407067 | 34407117 |
| chr12 | 34410197 | 34410247 |
| chr12 | 34411525 | 34411575 |
| chr12 | 34413190 | 34413240 |
| chr12 | 34415055 | 34415105 |
| chr12 | 34415954 | 34416004 |
| chr12 | 34418034 | 34418084 |
| chr12 | 34420630 | 34420680 |
| chr12 | 34421456 | 34421506 |
| chr12 | 34422708 | 34422758 |
| chr12 | 34423824 | 34423874 |
| chr12 | 34425483 | 34425533 |
| chr12 | 34427806 | 34427856 |
| chr12 | 34429072 | 34429122 |
| chr12 | 34436147 | 34436197 |
| chr12 | 34437394 | 34437444 |
| chr12 | 34442947 | 34442997 |
| chr12 | 34446019 | 34446069 |
| chr12 | 34447767 | 34447817 |
| chr12 | 34642395 | 34642445 |
| chr12 | 34644020 | 34644070 |
| chr12 | 34645336 | 34645386 |
| chr12 | 34646060 | 34646110 |
| chr12 | 34648711 | 34648761 |
| chr12 | 34649045 | 34649095 |
| chr12 | 34650600 | 34650650 |

Table 15M. Repeat Category Harlequin

| | | |
|---|---|---|
| chr17 | 41688008 | 41688058 |
| chr17 | 41692469 | 41692519 |
| chr17 | 48971947 | 48971997 |
| chr10 | 85592925 | 85592975 |
| chr10 | 85596390 | 85596440 |
| chr10 | 85596099 | 85596149 |
| chr10 | 85595842 | 85595892 |
| chr17 | 79083734 | 79083784 |
| chr17 | 79084761 | 79084811 |
| chr18 | 10593595 | 10593645 |
| chr10 | 95813106 | 95813156 |
| chr18 | 51771480 | 51771530 |
| chr18 | 67288389 | 67288439 |
| chr10 | 98985580 | 98985630 |
| chr19 | 6548612 | 6548662 |
| chr19 | 9679912 | 9679962 |
| chr19 | 19757388 | 19757438 |
| chr19 | 20217989 | 20218039 |
| chr19 | 20980348 | 20980398 |
| chr19 | 20987672 | 20987722 |
| chr19 | 20990030 | 20990080 |
| chr19 | 33790221 | 33790271 |
| chr19 | 57889039 | 57889089 |
| chr19 | 58094810 | 58094860 |
| chr19 | 58152373 | 58152423 |
| chr1 | 1301672 | 1301722 |
| chr1 | 35040144 | 35040194 |
| chr1 | 46167580 | 46167630 |
| chr1 | 91139029 | 91139079 |
| chr1 | 91141099 | 91141149 |
| chr1 | 91602320 | 91602370 |
| chr1 | 91607444 | 91607494 |
| chr1 | 165020614 | 165020664 |
| chr1 | 208944583 | 208944633 |
| chr1 | 217947911 | 217947961 |
| chr1 | 217952712 | 217952762 |
| chr1 | 224754280 | 224754330 |
| chr1 | 244966320 | 244966370 |
| chr20 | 15961266 | 15961316 |
| chr21 | 20292040 | 20292090 |
| chr21 | 32952678 | 32952728 |
| chr21 | 43248389 | 43248439 |
| chr21 | 43250115 | 43250165 |
| chrX | 9967063 | 9967113 |
| chrX | 47759915 | 47759965 |
| chrX | 47764086 | 47764136 |
| chrX | 52510059 | 52510109 |
| chrX | 52513675 | 52513725 |
| chrX | 113537475 | 113537525 |
| chrX | 113541216 | 113541266 |
| chrX | 113543646 | 113543696 |
| chrX | 113821185 | 113821235 |
| chrY | 14403477 | 14403527 |
| chrY | 17890936 | 17890986 |
| chr2 | 13687966 | 13688016 |
| chr2 | 13695140 | 13695190 |
| chr2 | 65089272 | 65089322 |
| chr2 | 65089956 | 65090006 |
| chr2 | 65090170 | 65090220 |
| chr2 | 65101094 | 65101144 |
| chr2 | 106217414 | 106217464 |
| chr2 | 188583849 | 188583899 |
| chr2 | 209182252 | 209182302 |
| chr2 | 214474051 | 214474101 |
| chr2 | 214477356 | 214477406 |
| chr3 | 865355 | 865405 |
| chr3 | 20387292 | 20387342 |
| chr3 | 121351668 | 121351718 |
| chr11 | 60639025 | 60639075 |
| chr3 | 135060147 | 135060197 |
| chr4 | 8628791 | 8628841 |
| chr4 | 8629462 | 8629512 |
| chr4 | 8633179 | 8633229 |
| chr4 | 22351979 | 22352029 |
| chr4 | 36805197 | 36805247 |
| chr4 | 70232770 | 70232820 |
| chr4 | 70232602 | 70232652 |
| chr4 | 93425519 | 93425569 |
| chr4 | 159727410 | 159727460 |
| chr4 | 174716255 | 174716305 |
| chr5 | 42859327 | 42859377 |
| chr5 | 64486100 | 64486150 |
| chr5 | 64490718 | 64490768 |
| chr5 | 81649376 | 81649426 |
| chr5 | 81653083 | 81653133 |
| chr5 | 116118192 | 116118242 |
| chr5 | 138935066 | 138935116 |
| chr11 | 74482411 | 74482461 |
| chr5 | 158774642 | 158774692 |
| chr5 | 162727134 | 162727184 |
| chr11 | 81389164 | 81389214 |
| chr6 | 26183592 | 26183642 |
| chr6 | 35016783 | 35016833 |
| chr6 | 47649941 | 47649991 |
| chr6 | 75174105 | 75174155 |
| chr6 | 111653619 | 111653669 |
| chr11 | 97805236 | 97805286 |
| chr7 | 4805043 | 4805093 |
| chr7 | 4806585 | 4806635 |
| chr7 | 4807691 | 4807741 |
| chr7 | 10711055 | 10711105 |
| chr7 | 24577109 | 24577159 |
| chr7 | 30590054 | 30590104 |
| chr7 | 100048567 | 100048617 |
| chr7 | 102353546 | 102353596 |
| chr7 | 127795349 | 127795399 |
| chr7 | 136364655 | 136364705 |
| chr7 | 136369064 | 136369114 |
| chr8 | 41468393 | 41468443 |
| chr8 | 41471654 | 41471704 |
| chr8 | 54486289 | 54486339 |
| chr8 | 54486391 | 54486441 |
| chr8 | 56675871 | 56675921 |
| chr8 | 59653843 | 59653893 |
| chr8 | 144863318 | 144863368 |
| chr8 | 144866035 | 144866085 |
| chr8 | 144868006 | 144868056 |
| chr12 | 8492990 | 8493040 |
| chr12 | 32071135 | 32071185 |
| chr12 | 48414803 | 48414853 |
| chr12 | 71498252 | 71498302 |
| chr12 | 71503639 | 71503689 |
| chr10 | 30769582 | 30769632 |
| chr13 | 21259309 | 21259359 |
| chr13 | 65771389 | 65771439 |
| chr13 | 65777169 | 65777219 |
| chr10 | 60835403 | 60835453 |
| chr10 | 60869487 | 60869537 |
| chr10 | 73044293 | 73044343 |

Table 15N. Repeat Category HERV9

| | | |
|---|---|---|
| chr17 | 73500503 | 73500553 |
| chr17 | 73502106 | 73502156 |
| chr10 | 90831550 | 90831600 |
| chr18 | 4806200 | 4806250 |
| chr18 | 5076788 | 5076838 |
| chr10 | 96197497 | 96197547 |
| chr19 | 8350946 | 8350996 |
| chr19 | 18555519 | 18555569 |
| chr19 | 44005505 | 44005555 |
| chr19 | 44005795 | 44005845 |
| chr19 | 44006124 | 44006174 |
| chr19 | 44006438 | 44006488 |
| chr19 | 45143853 | 45143903 |
| chr19 | 45145464 | 45145514 |
| chr1 | 6330271 | 6330321 |
| chr1 | 6333673 | 6333723 |
| chr10 | 117925907 | 117925957 |
| chr1 | 58003791 | 58003841 |
| chr1 | 62590722 | 62590772 |
| chr1 | 62591082 | 62591132 |
| chr1 | 62591469 | 62591519 |
| chr1 | 66262007 | 66262057 |
| chr1 | 102673199 | 102673249 |
| chr1 | 105009493 | 105009543 |
| chr1 | 119061757 | 119061807 |
| chr1 | 119063336 | 119063386 |
| chr1 | 151862317 | 151862367 |
| chr1 | 172315523 | 172315573 |
| chr1 | 196350746 | 196350796 |
| chr20 | 40225452 | 40225502 |
| chr21 | 29648303 | 29648353 |
| chrX | 25877374 | 25877424 |
| chrX | 102899430 | 102899480 |
| chrX | 113507179 | 113507229 |
| chrX | 114159564 | 114159614 |
| chrX | 116169376 | 116169426 |
| chrY | 15010206 | 15010256 |
| chrY | 15714739 | 15714789 |
| chr2 | 9058138 | 9058188 |
| chr2 | 21299682 | 21299732 |
| chr2 | 33252847 | 33252897 |
| chr2 | 33256063 | 33256113 |
| chr2 | 47302706 | 47302756 |
| chr2 | 47303895 | 47303945 |
| chr2 | 63524436 | 63524486 |
| chr2 | 63525488 | 63525538 |
| chr2 | 63527836 | 63527886 |
| chr11 | 23922340 | 23922390 |
| chr11 | 23927165 | 23927215 |
| chr2 | 164126675 | 164126725 |
| chr2 | 184953385 | 184953435 |
| chr11 | 38102714 | 38102764 |
| chr2 | 203564551 | 203564601 |
| chr11 | 42861632 | 42861682 |
| chr2 | 242127537 | 242127587 |
| chr2 | 242127273 | 242127323 |
| chr3 | 9595278 | 9595328 |
| chr3 | 9599612 | 9599662 |
| chr3 | 20287051 | 20287101 |
| chr3 | 33460626 | 33460676 |
| chr3 | 38441934 | 38441984 |
| chr3 | 49219830 | 49219880 |
| chr3 | 51843981 | 51844031 |
| chr3 | 56710325 | 56710375 |
| chr3 | 96634754 | 96634804 |
| chr3 | 149610248 | 149610298 |
| chr3 | 150323777 | 150323827 |
| chr3 | 152709083 | 152709133 |
| chr3 | 153600090 | 153600140 |
| chr3 | 163613973 | 163614023 |
| chr3 | 164841947 | 164841997 |
| chr4 | 24997332 | 24997382 |
| chr4 | 24998346 | 24998396 |
| chr4 | 45449397 | 45449447 |
| chr4 | 59240331 | 59240381 |
| chr4 | 63922678 | 63922728 |
| chr4 | 71006856 | 71006906 |
| chr4 | 77429274 | 77429324 |
| chr4 | 103010781 | 103010831 |
| chr4 | 132227868 | 132227918 |
| chr4 | 160157147 | 160157197 |
| chr4 | 160163810 | 160163860 |
| chr11 | 67163610 | 67163660 |
| chr5 | 49682936 | 49682986 |
| chr5 | 54927246 | 54927296 |
| chr5 | 96727232 | 96727282 |
| chr5 | 125633601 | 125633651 |
| chr5 | 125634821 | 125634871 |
| chr5 | 130046591 | 130046641 |
| chr5 | 144256216 | 144256266 |
| chr5 | 157288569 | 157288619 |
| chr5 | 157294830 | 157294880 |
| chr6 | 9831174 | 9831224 |
| chr6 | 19380476 | 19380526 |
| chr11 | 84534139 | 84534189 |
| chr11 | 85044385 | 85044435 |
| chr6 | 38631973 | 38632023 |
| chr6 | 38638804 | 38638854 |
| chr6 | 52873056 | 52873106 |
| chr6 | 64039538 | 64039588 |
| chr6 | 64731695 | 64731745 |
| chr6 | 74609656 | 74609706 |
| chr6 | 116458435 | 116458485 |
| chr6 | 116459467 | 116459517 |
| chr11 | 96045911 | 96045961 |
| chr6 | 141485259 | 141485309 |
| chr11 | 100648542 | 100648592 |
| chr11 | 102751565 | 102751615 |
| chr7 | 23637191 | 23637241 |
| chr7 | 36984863 | 36984913 |
| chr7 | 63552127 | 63552177 |
| chr7 | 81883546 | 81883596 |
| chr7 | 143513210 | 143513260 |
| chr8 | 4429220 | 4429270 |
| chr8 | 11177208 | 11177258 |
| chr8 | 27371989 | 27372039 |
| chr8 | 29184987 | 29185037 |
| chr8 | 50618557 | 50618607 |
| chr8 | 73350754 | 73350804 |
| chr8 | 74874887 | 74874937 |
| chr8 | 100132498 | 100132548 |
| chr9 | 22426217 | 22426267 |
| chr9 | 26816413 | 26816463 |
| chr9 | 84228794 | 84228844 |
| chr12 | 1695379 | 1695429 |
| chr12 | 8833152 | 8833202 |
| chr12 | 18083947 | 18083997 |
| chr12 | 20821865 | 20821915 |
| chr12 | 29429505 | 29429555 |
| chr12 | 31981044 | 31981094 |
| chr12 | 81269085 | 81269135 |
| chr12 | 106108037 | 106108087 |
| chr12 | 106718560 | 106718610 |
| chr10 | 31194356 | 31194406 |
| chr13 | 21898560 | 21898610 |
| chr13 | 98429093 | 98429143 |
| chr13 | 98636368 | 98636418 |
| chr13 | 106246891 | 106246941 |
| chr13 | 109546410 | 109546460 |
| chr14 | 22095947 | 22095997 |
| chr14 | 79969950 | 79970000 |
| chr10 | 55400187 | 55400237 |
| chr10 | 58945869 | 58945919 |
| chr15 | 42870466 | 42870516 |
| chr15 | 43019410 | 43019460 |
| chr10 | 65888931 | 65888981 |
| chr16 | 21608058 | 21608108 |
| chr16 | 21613682 | 21613732 |

Table 15O. Repeat Category HERVFH21

| | | |
|---|---|---|
| chr19 | 19916932 | 19916982 |
| chr1 | 6333673 | 6333723 |
| chr1 | 6334500 | 6334550 |
| chr1 | 6336594 | 6336644 |
| chr1 | 6337256 | 6337306 |
| chr1 | 6338247 | 6338297 |
| chr1 | 6338700 | 6338750 |
| chr1 | 8691734 | 8691784 |
| chr1 | 8695896 | 8695946 |
| chr1 | 8695658 | 8695708 |
| chr1 | 36224419 | 36224469 |
| chr1 | 36227399 | 36227449 |
| chr1 | 36230083 | 36230133 |
| chr1 | 144512253 | 144512303 |
| chr1 | 154460528 | 154460578 |
| chr1 | 154465550 | 154465600 |
| chr11 | 3338103 | 3338153 |
| chr11 | 3342619 | 3342669 |
| chr11 | 5915252 | 5915302 |
| chr11 | 5917222 | 5917272 |
| chr11 | 5918320 | 5918370 |
| chrY | 13412083 | 13412133 |
| chrY | 13416775 | 13416825 |
| chr2 | 62126818 | 62126868 |
| chr11 | 31594478 | 31594528 |
| chr11 | 58787567 | 58787617 |
| chr3 | 142738000 | 142738050 |
| chr3 | 142741496 | 142741546 |
| chr3 | 152845697 | 152845747 |
| chr3 | 165063501 | 165063551 |
| chr5 | 29436941 | 29436991 |
| chr5 | 165536408 | 165536458 |
| chr6 | 17428373 | 17428423 |
| chr6 | 17430481 | 17430531 |
| chr6 | 17431369 | 17431419 |
| chr6 | 17435075 | 17435125 |
| chr7 | 12373055 | 12373105 |
| chr7 | 12373469 | 12373519 |
| chr7 | 12378141 | 12378191 |
| chr7 | 63707360 | 63707410 |
| chr7 | 63710987 | 63711037 |
| chr7 | 64353237 | 64353287 |
| chr7 | 64354507 | 64354557 |
| chr7 | 152500346 | 152500396 |
| chr7 | 152501738 | 152501788 |
| chr8 | 48951898 | 48951948 |
| chr8 | 48953354 | 48953404 |
| chr8 | 48960445 | 48960495 |
| chr12 | 111641372 | 111641422 |
| chr14 | 100691478 | 100691528 |
| chr14 | 100703440 | 100703490 |
| chr14 | 100704576 | 100704626 |
| chr16 | 30590321 | 30590371 |
| chr16 | 30592221 | 30592271 |
| chr16 | 30593884 | 30593934 |
| chr10 | 73065984 | 73066034 |
| chr10 | 73066226 | 73066276 |
| chr10 | 73066517 | 73066567 |
| chr10 | 73066592 | 73066642 |
| chr10 | 73067037 | 73067087 |

Table 15P. Repeat Category HERVK11

| | | |
|---|---|---|
| chr18 | 70123608 | 70123658 |
| chr10 | 104264539 | 104264589 |
| chr10 | 104264619 | 104264669 |
| chr10 | 104264710 | 104264760 |
| chr10 | 104267695 | 104267745 |
| chr1 | 109545458 | 109545508 |
| chr1 | 109549553 | 109549603 |
| chr1 | 157877337 | 157877387 |
| chr1 | 208652478 | 208652528 |
| chr22 | 18297951 | 18298001 |
| chr22 | 18299679 | 18299729 |
| chr22 | 18302389 | 18302439 |
| chrX | 34176612 | 34176662 |
| chrX | 45394539 | 45394589 |
| chrX | 55947070 | 55947120 |
| chr2 | 64192256 | 64192306 |
| chr2 | 77315804 | 77315854 |
| chr2 | 101481794 | 101481844 |
| chr3 | 131491371 | 131491421 |
| chr3 | 168222061 | 168222111 |
| chr4 | 4113608 | 4113658 |
| chr4 | 81207097 | 81207147 |
| chr4 | 81206931 | 81206981 |
| chr4 | 141132783 | 141132833 |
| chr11 | 67242926 | 67242976 |
| chr11 | 67247088 | 67247138 |
| chr4 | 166247883 | 166247933 |
| chr4 | 166249950 | 166250000 |
| chr5 | 54688468 | 54688518 |
| chr6 | 106108380 | 106108430 |
| chr6 | 121344741 | 121344791 |
| chr7 | 50387172 | 50387222 |
| chr8 | 29195245 | 29195295 |
| chr8 | 89645119 | 89645169 |
| chr9 | 31762122 | 31762172 |
| chr9 | 110540653 | 110540703 |
| chr12 | 82998661 | 82998711 |
| chr12 | 83004551 | 83004601 |
| chr16 | 3969546 | 3969596 |

Table 15Q. Repeat Category HERVK14

| | | |
|---|---|---|
| chr17 | 73616199 | 73616249 |
| chr17 | 73621355 | 73621405 |
| chr17 | 73623410 | 73623460 |
| chr19 | 12424237 | 12424287 |
| chr19 | 24145792 | 24145842 |
| chr19 | 56594884 | 56594934 |
| chr19 | 63101807 | 63101857 |
| chr19 | 63104294 | 63104344 |
| chr19 | 63106137 | 63106187 |
| chr1 | 62014127 | 62014177 |
| chr1 | 203009281 | 203009331 |
| chr1 | 203009106 | 203009156 |
| chr1 | 222955002 | 222955052 |
| chr1 | 222959363 | 222959413 |
| chr1 | 222961831 | 222961881 |
| chr1 | 222962980 | 222963030 |
| chr1 | 244216642 | 244216692 |
| chr22 | 24135046 | 24135096 |
| chr22 | 29396210 | 29396260 |
| chr22 | 29398342 | 29398392 |
| chr22 | 29400220 | 29400270 |
| chrX | 116182682 | 116182732 |
| chrX | 116188297 | 116188347 |
| chrY | 7697268 | 7697318 |
| chrY | 13455713 | 13455763 |
| chrY | 22454225 | 22454275 |
| chrY | 22458742 | 22458792 |
| chrY | 22458853 | 22458903 |
| chr2 | 82225138 | 82225188 |
| chr2 | 128468837 | 128468887 |
| chr2 | 181124049 | 181124099 |
| chr2 | 217138311 | 217138361 |
| chr11 | 55875311 | 55875361 |
| chr3 | 173758124 | 173758174 |
| chr3 | 173759285 | 173759335 |
| chr4 | 176606965 | 176607015 |
| chr5 | 23733108 | 23733158 |
| chr5 | 24353855 | 24353905 |
| chr5 | 31440433 | 31440483 |
| chr5 | 31444280 | 31444330 |
| chr11 | 73382443 | 73382493 |
| chr6 | 48888931 | 48888981 |
| chr6 | 48892648 | 48892698 |
| chr6 | 79559788 | 79559838 |
| chr6 | 126213455 | 126213505 |
| chr6 | 134906416 | 134906466 |
| chr6 | 166668349 | 166668399 |
| chr7 | 8801179 | 8801229 |
| chr7 | 8804044 | 8804094 |
| chr11 | 120645173 | 120645223 |
| chr9 | 29649846 | 29649896 |
| chr9 | 29770178 | 29770228 |
| chr12 | 45585167 | 45585217 |
| chr12 | 47190537 | 47190587 |
| chr12 | 47190332 | 47190382 |
| chr12 | 47191786 | 47191836 |
| chr12 | 47191649 | 47191699 |
| chr10 | 42266499 | 42266549 |
| chr13 | 63081280 | 63081330 |
| chr13 | 91390124 | 91390174 |
| chr15 | 60222264 | 60222314 |
| chr15 | 74275229 | 74275279 |
| chr16 | 19379055 | 19379105 |
| chr10 | 73065984 | 73066034 |

Table 15R. Repeat Category HERVK22

| | | |
|---|---|---|
| chr18 | 25255495 | 25255545 |
| chr19 | 23532349 | 23532399 |
| chr19 | 32928882 | 32928932 |
| chr19 | 32930010 | 32930060 |
| chr19 | 32929918 | 32929968 |
| chr19 | 32932324 | 32932374 |
| chr19 | 47892385 | 47892435 |
| chr19 | 47892649 | 47892699 |
| chr19 | 47893498 | 47893548 |
| chr19 | 47893919 | 47893969 |
| chr19 | 47894016 | 47894066 |
| chr19 | 47894490 | 47894540 |
| chr19 | 57854025 | 57854075 |
| chr1 | 86396110 | 86396160 |
| chr1 | 153375315 | 153375365 |
| chr1 | 156138608 | 156138658 |
| chr1 | 158096865 | 158096915 |
| chr1 | 168460105 | 168460155 |
| chr1 | 239322614 | 239322664 |
| chr20 | 25602563 | 25602613 |
| chrX | 25598216 | 25598266 |
| chrX | 62872118 | 62872168 |
| chrX | 63282890 | 63282940 |
| chrX | 92519863 | 92519913 |
| chrX | 92519608 | 92519658 |
| chrX | 92522265 | 92522315 |
| chrX | 145018136 | 145018186 |
| chrX | 145020894 | 145020944 |
| chrX | 147470265 | 147470315 |
| chrY | 20926896 | 20926946 |
| chr2 | 71590282 | 71590332 |
| chr2 | 71595325 | 71595375 |
| chr2 | 128222110 | 128222160 |
| chr2 | 167668621 | 167668671 |
| chr2 | 167669823 | 167669873 |
| chr2 | 232163770 | 232163820 |
| chr2 | 232168171 | 232168221 |
| chr11 | 58470665 | 58470715 |
| chr11 | 58470350 | 58470400 |
| chr3 | 171116443 | 171116493 |
| chr3 | 186599533 | 186599583 |
| chr4 | 136786588 | 136786638 |
| chr4 | 136788228 | 136788278 |
| chr5 | 26892790 | 26892840 |
| chr5 | 37132931 | 37132981 |
| chr11 | 80470448 | 80470498 |
| chr11 | 80475408 | 80475458 |
| chr6 | 26367620 | 26367670 |
| chr6 | 56986133 | 56986183 |
| chr6 | 65010255 | 65010305 |
| chr6 | 101439809 | 101439859 |
| chr11 | 95355336 | 95355386 |
| chr11 | 95360550 | 95360600 |
| chr7 | 39663241 | 39663291 |
| chr7 | 106587234 | 106587284 |
| chr8 | 47583088 | 47583138 |
| chr8 | 89580634 | 89580684 |
| chr8 | 89580484 | 89580534 |
| chr9 | 98509088 | 98509138 |
| chr9 | 98510946 | 98510996 |
| chr12 | 8602302 | 8602352 |
| chr12 | 41122714 | 41122764 |
| chr12 | 41126148 | 41126198 |
| chr12 | 68011122 | 68011172 |
| chr12 | 87080333 | 87080383 |
| chr12 | 94342775 | 94342825 |
| chr12 | 99060281 | 99060331 |
| chr12 | 99060182 | 99060232 |
| chr12 | 99061198 | 99061248 |
| chr13 | 23299768 | 23299818 |
| chr13 | 23299820 | 23299870 |
| chr10 | 43084179 | 43084229 |
| chr10 | 43084465 | 43084515 |
| chr10 | 43084735 | 43084785 |
| chr10 | 43085253 | 43085303 |
| chr10 | 43085538 | 43085588 |
| chr14 | 78921071 | 78921121 |
| chr14 | 105126835 | 105126885 |
| chr14 | 105126736 | 105126786 |
| chr15 | 37021929 | 37021979 |
| chr15 | 37021655 | 37021705 |
| chr15 | 47847615 | 47847665 |
| chr15 | 52895510 | 52895560 |

Table 15S. Repeat Category HERVK9

| | | |
|---|---|---|
| chr17 | 47728182 | 47728232 |
| chr19 | 9426058 | 9426108 |
| chr19 | 44123445 | 44123495 |
| chr19 | 44123551 | 44123601 |
| chr1 | 19337955 | 19338005 |
| chr10 | 120038656 | 120038706 |
| chr1 | 48355087 | 48355137 |
| chr1 | 54466355 | 54466405 |
| chr1 | 54470375 | 54470425 |
| chr1 | 140694238 | 140694288 |
| chr1 | 141061552 | 141061602 |
| chr1 | 141331391 | 141331441 |
| chr20 | 23443196 | 23443246 |
| chrX | 73410922 | 73410972 |
| chrX | 73410727 | 73410777 |
| chrX | 73414467 | 73414517 |
| chrX | 78054225 | 78054275 |
| chrY | 14373702 | 14373752 |
| chr2 | 20107539 | 20107589 |
| chr2 | 20110072 | 20110122 |
| chr2 | 48587291 | 48587341 |
| chr2 | 56921191 | 56921241 |
| chr2 | 153237365 | 153237415 |
| chr2 | 153239665 | 153239715 |
| chr2 | 184120362 | 184120412 |
| chr2 | 219179971 | 219180021 |
| chr3 | 38423302 | 38423352 |
| chr3 | 38425563 | 38425613 |
| chr3 | 130945368 | 130945418 |
| chr3 | 130945427 | 130945477 |
| chr3 | 186056639 | 186056689 |
| chr4 | 92565890 | 92565940 |
| chr4 | 155192336 | 155192386 |
| chr4 | 186666592 | 186666642 |
| chr4 | 186668148 | 186668198 |
| chr5 | 295513 | 295563 |
| chr5 | 1636141 | 1636191 |
| chr5 | 34832555 | 34832605 |
| chr5 | 121272595 | 121272645 |
| chr6 | 6013296 | 6013346 |
| chr6 | 45250837 | 45250887 |
| chr6 | 67036583 | 67036633 |
| chr6 | 93903878 | 93903928 |
| chr7 | 42416386 | 42416436 |
| chr7 | 43644071 | 43644121 |
| chr7 | 45507361 | 45507411 |
| chr7 | 55959316 | 55959366 |
| chr7 | 154422874 | 154422924 |
| chr8 | 53946006 | 53946056 |
| chr8 | 93206494 | 93206544 |
| chr8 | 146103607 | 146103657 |
| chr9 | 129832348 | 129832398 |
| chr9 | 129833786 | 129833836 |
| chr12 | 37880721 | 37880771 |
| chr12 | 49178322 | 49178372 |
| chr12 | 50419640 | 50419690 |
| chr12 | 53957670 | 53957720 |
| chr12 | 93252455 | 93252505 |
| chr12 | 104200372 | 104200422 |
| chr12 | 104204812 | 104204862 |
| chr13 | 19056838 | 19056888 |
| chr13 | 21275891 | 21275941 |
| chr10 | 42276366 | 42276416 |
| chr14 | 55823217 | 55823267 |
| chr14 | 101691027 | 101691077 |
| chr14 | 101692650 | 101692700 |
| chr15 | 21493283 | 21493333 |
| chr15 | 87508917 | 87508967 |
| chr15 | 87512657 | 87512707 |
| chr10 | 69967970 | 69968020 |
| chr10 | 69969376 | 69969426 |
| chr10 | 69971039 | 69971089 |
| chr10 | 69972473 | 69972523 |
| chr16 | 3127347 | 3127397 |
| chr16 | 3131557 | 3131607 |
| chr17 | 21145322 | 21145372 |
| chr17 | 28459509 | 28459559 |

Table 15T. Repeat Category HERVL

| | | |
|---|---|---|
| chr17 | 42575356 | 42575406 |
| chr17 | 59112058 | 59112108 |
| chr18 | 2823661 | 2823711 |
| chr10 | 4757976 | 4758026 |
| chr18 | 27630945 | 27630995 |
| chr18 | 33712437 | 33712487 |
| chr18 | 39314666 | 39314716 |
| chr18 | 39331130 | 39331180 |
| chr18 | 39330976 | 39331026 |
| chr18 | 62808467 | 62808517 |
| chr18 | 67332499 | 67332549 |
| chr19 | 9710880 | 9710930 |
| chr19 | 12058125 | 12058175 |
| chr19 | 19834257 | 19834307 |
| chr19 | 20023775 | 20023825 |
| chr19 | 20041410 | 20041460 |
| chr19 | 20044959 | 20045009 |
| chr19 | 20125977 | 20126027 |
| chr19 | 43476713 | 43476763 |
| chr19 | 43478244 | 43478294 |
| chr19 | 54422177 | 54422227 |
| chr19 | 54476385 | 54476435 |
| chr19 | 56996534 | 56996584 |
| chr19 | 58225411 | 58225461 |
| chr19 | 58226657 | 58226707 |
| chr19 | 58701580 | 58701630 |
| chr1 | 4320728 | 4320778 |
| chr1 | 8706643 | 8706693 |
| chr1 | 15683769 | 15683819 |
| chr1 | 28673990 | 28674040 |
| chr1 | 31486513 | 31486563 |
| chr1 | 31485821 | 31485871 |
| chr1 | 35190033 | 35190083 |
| chr1 | 79938727 | 79938777 |
| chr1 | 79938965 | 79939015 |
| chr1 | 79939292 | 79939342 |
| chr1 | 79939829 | 79939879 |
| chr1 | 79940070 | 79940120 |
| chr1 | 85280755 | 85280805 |
| chr1 | 88529129 | 88529179 |
| chr1 | 107467789 | 107467839 |
| chr1 | 111132109 | 111132159 |
| chr1 | 113907964 | 113908014 |
| chr1 | 117710217 | 117710267 |
| chr1 | 117710017 | 117710067 |
| chr1 | 158619615 | 158619665 |
| chr1 | 158619006 | 158619056 |
| chr1 | 185549613 | 185549663 |
| chr1 | 196235232 | 196235282 |
| chr1 | 204680138 | 204680188 |
| chr1 | 204680639 | 204680689 |
| chr1 | 204681551 | 204681601 |
| chr1 | 223698589 | 223698639 |
| chr1 | 223705042 | 223705092 |
| chr1 | 244141539 | 244141589 |
| chr1 | 244143609 | 244143659 |
| chr1 | 244255088 | 244255138 |
| chr20 | 2651528 | 2651578 |
| chr20 | 2968225 | 2968275 |
| chr20 | 33538013 | 33538063 |
| chr10 | 133920653 | 133920703 |
| chr10 | 134663497 | 134663547 |
| chr21 | 29145010 | 29145060 |
| chr21 | 29156309 | 29156359 |
| chr22 | 17463493 | 17463543 |
| chr22 | 21067166 | 21067216 |
| chr22 | 21432174 | 21432224 |
| chr22 | 31477700 | 31477750 |
| chr22 | 40454531 | 40454581 |
| chr22 | 44231708 | 44231758 |
| chrX | 4151370 | 4151420 |
| chrX | 4153605 | 4153655 |
| chrX | 8471941 | 8471991 |
| chrX | 19416489 | 19416539 |
| chrX | 37636313 | 37636363 |
| chrX | 39716439 | 39716489 |
| chrX | 39720531 | 39720581 |
| chrX | 46518193 | 46518243 |
| chrX | 71958452 | 71958502 |
| chrX | 84928330 | 84928380 |
| chrX | 101309114 | 101309164 |
| chrX | 101885783 | 101885833 |
| chrX | 106492964 | 106493014 |
| chrX | 125725267 | 125725317 |
| chrX | 147322032 | 147322082 |
| chrX | 147321813 | 147321863 |
| chrX | 147538903 | 147538953 |
| chrY | 8716577 | 8716627 |
| chrY | 20979665 | 20979715 |
| chrY | 20981332 | 20981382 |
| chr2 | 24514470 | 24514520 |
| chr2 | 35988290 | 35988340 |
| chr11 | 21798360 | 21798410 |
| chr2 | 70405449 | 70405499 |
| chr2 | 80381322 | 80381372 |
| chr2 | 100516923 | 100516973 |
| chr2 | 113434220 | 113434270 |
| chr2 | 128683735 | 128683785 |
| chr2 | 162892086 | 162892136 |
| chr11 | 39114823 | 39114873 |
| chr11 | 39330117 | 39330167 |
| chr2 | 224369830 | 224369880 |
| chr2 | 242278366 | 242278416 |
| chr2 | 242279651 | 242279701 |
| chr3 | 3847958 | 3848008 |
| chr3 | 12728266 | 12728316 |
| chr3 | 19906213 | 19906263 |
| chr3 | 46818060 | 46818110 |
| chr3 | 48006886 | 48006936 |
| chr11 | 56165381 | 56165431 |
| chr3 | 106644031 | 106644081 |
| chr3 | 119403971 | 119404021 |
| chr4 | 966070 | 966120 |
| chr4 | 2773936 | 2773986 |
| chr4 | 17676139 | 17676189 |
| chr4 | 29509997 | 29510047 |
| chr4 | 62297442 | 62297492 |
| chr4 | 62297235 | 62297285 |
| chr4 | 67648786 | 67648836 |
| chr4 | 84369941 | 84369991 |
| chr4 | 95536618 | 95536668 |
| chr4 | 102996809 | 102996859 |
| chr4 | 104125960 | 104126010 |
| chr4 | 133470625 | 133470675 |
| chr4 | 135587729 | 135587779 |
| chr4 | 138789336 | 138789386 |
| chr4 | 139626989 | 139627039 |
| chr4 | 140941457 | 140941507 |
| chr4 | 166432660 | 166432710 |
| chr4 | 166432827 | 166432877 |
| chr4 | 166625111 | 166625161 |
| chr4 | 166625966 | 166626016 |
| chr4 | 179755826 | 179755876 |
| chr4 | 187675052 | 187675102 |
| chr5 | 12392304 | 12392354 |
| chr5 | 18342099 | 18342149 |
| chr5 | 18839149 | 18839199 |
| chr5 | 23265007 | 23265057 |
| chr5 | 26820059 | 26820109 |
| chr5 | 43196076 | 43196126 |
| chr5 | 43204176 | 43204226 |
| chr5 | 56535353 | 56535403 |
| chr5 | 74168616 | 74168666 |
| chr5 | 101174232 | 101174282 |
| chr5 | 101847480 | 101847530 |
| chr5 | 106285518 | 106285568 |
| chr5 | 106287716 | 106287766 |
| chr5 | 106325045 | 106325095 |
| chr5 | 111453449 | 111453499 |
| chr11 | 73979736 | 73979786 |
| chr5 | 152031779 | 152031829 |
| chr5 | 160346629 | 160346679 |
| chr5 | 160346343 | 160346393 |
| chr5 | 160403079 | 160403129 |
| chr6 | 3876977 | 3877027 |
| chr11 | 77590133 | 77590183 |
| chr6 | 10244944 | 10244994 |
| chr6 | 21466481 | 21466531 |
| chr6 | 28077381 | 28077431 |
| chr6 | 28432162 | 28432212 |
| chr6 | 28431869 | 28431919 |
| chr6 | 29759374 | 29759424 |
| chr11 | 87854950 | 87855000 |
| chr6 | 71271736 | 71271786 |
| chr6 | 72895922 | 72895972 |
| chr11 | 90082072 | 90082122 |
| chr6 | 80018768 | 80018818 |
| chr6 | 80018515 | 80018565 |
| chr6 | 90250211 | 90250261 |
| chr6 | 94547928 | 94547978 |
| chr6 | 105710211 | 105710261 |
| chr6 | 122896591 | 122896641 |
| chr11 | 96400830 | 96400880 |
| chr6 | 132473418 | 132473468 |
| chr6 | 133665465 | 133665515 |
| chr11 | 97183407 | 97183457 |
| chr6 | 135174097 | 135174147 |
| chr6 | 153503258 | 153503308 |
| chr6 | 167387267 | 167387317 |
| chr7 | 13233606 | 13233656 |
| chr7 | 81443708 | 81443758 |
| chr7 | 99741950 | 99742000 |
| chr7 | 116598271 | 116598321 |
| chr7 | 118383067 | 118383117 |
| chr7 | 148144855 | 148144905 |
| chr7 | 156587986 | 156588036 |
| chr8 | 2760276 | 2760326 |
| chr8 | 5075110 | 5075160 |
| chr8 | 5501858 | 5501908 |
| chr8 | 13448334 | 13448384 |
| chr8 | 55150706 | 55150756 |
| chr8 | 78786789 | 78786839 |
| chr8 | 95268153 | 95268203 |
| chr8 | 122480079 | 122480129 |
| chr9 | 20504875 | 20504925 |
| chr9 | 75026755 | 75026805 |
| chr9 | 76145955 | 76146005 |
| chr9 | 76145889 | 76145939 |
| chr9 | 87390311 | 87390361 |
| chr9 | 92073065 | 92073115 |
| chr9 | 92072981 | 92073031 |
| chr9 | 110517709 | 110517759 |
| chr9 | 132828175 | 132828225 |
| chr9 | 134843242 | 134843292 |
| chr9 | 134960777 | 134960827 |
| chr12 | 8040415 | 8040465 |
| chr12 | 8043462 | 8043512 |
| chr12 | 14342962 | 14343012 |
| chr12 | 17013608 | 17013658 |
| chr12 | 31767862 | 31767912 |
| chr12 | 49002379 | 49002429 |
| chr12 | 49438886 | 49438936 |
| chr12 | 86591717 | 86591767 |
| chr12 | 119458129 | 119458179 |
| chr10 | 41968502 | 41968552 |
| chr13 | 58869201 | 58869251 |
| chr13 | 88963769 | 88963819 |
| chr14 | 28988650 | 28988700 |
| chr14 | 69917591 | 69917641 |
| chr14 | 69921435 | 69921485 |
| chr10 | 52511442 | 52511492 |
| chr14 | 75775508 | 75775558 |
| chr10 | 55208723 | 55208773 |
| chr14 | 98627075 | 98627125 |
| chr14 | 101717064 | 101717114 |
| chr14 | 101738835 | 101738885 |
| chr10 | 59721874 | 59721924 |
| chr15 | 19941143 | 19941193 |
| chr15 | 21315109 | 21315159 |
| chr15 | 51919168 | 51919218 |
| chr15 | 51918908 | 51918958 |
| chr15 | 77699950 | 77700000 |
| chr15 | 91370024 | 91370074 |
| chr10 | 71757549 | 71757599 |
| chr16 | 25044777 | 25044827 |
| chr16 | 89029919 | 89029969 |
| chr17 | 4230259 | 4230309 |
| chr17 | 33880226 | 33880276 |

Table 15U. Repeat Category HERVL18

| | | |
|---|---|---|
| chr17 | 59112058 | 59112108 |
| chr18 | 39314666 | 39314716 |
| chr19 | 12058125 | 12058175 |
| chr1 | 79938727 | 79938777 |
| chr1 | 79938965 | 79939015 |
| chr1 | 79939292 | 79939342 |
| chr1 | 79939829 | 79939879 |
| chr1 | 79940070 | 79940120 |
| chr1 | 117710217 | 117710267 |
| chr1 | 117710017 | 117710067 |
| chr1 | 204680138 | 204680188 |
| chr1 | 204680639 | 204680689 |
| chr1 | 204681551 | 204681601 |
| chr2 | 35988290 | 35988340 |
| chr11 | 21798360 | 21798410 |
| chr2 | 70405449 | 70405499 |
| chr2 | 128683735 | 128683785 |
| chr11 | 39330117 | 39330167 |
| chr4 | 62297442 | 62297492 |
| chr4 | 62297235 | 62297285 |
| chr4 | 179755826 | 179755876 |
| chr5 | 18839149 | 18839199 |
| chr5 | 101174232 | 101174282 |
| chr5 | 106285518 | 106285568 |
| chr5 | 106287716 | 106287766 |
| chr11 | 73979736 | 73979786 |
| chr5 | 160346629 | 160346679 |
| chr5 | 160346343 | 160346393 |
| chr6 | 28077381 | 28077431 |
| chr6 | 28432162 | 28432212 |
| chr6 | 28431869 | 28431919 |
| chr11 | 87854950 | 87855000 |
| chr6 | 80018768 | 80018818 |
| chr6 | 80018515 | 80018565 |
| chr6 | 135174097 | 135174147 |
| chr6 | 153503258 | 153503308 |
| chr7 | 81443708 | 81443758 |
| chr13 | 88963769 | 88963819 |
| chr14 | 28988650 | 28988700 |
| chr10 | 59721874 | 59721924 |
| chr15 | 51919168 | 51919218 |
| chr15 | 51918908 | 51918958 |

Table 15V. Repeat Category HERVP71A

| | | |
|---|---|---|
| chr10 | 103617621 | 103617671 |
| chr10 | 103618933 | 103618983 |
| chr10 | 103621736 | 103621786 |
| chr10 | 105492007 | 105492057 |
| chr1 | 71341164 | 71341214 |
| chr1 | 74230932 | 74230982 |
| chr1 | 244329852 | 244329902 |
| chr1 | 244470329 | 244470379 |
| chrX | 98597408 | 98597458 |
| chrX | 98603436 | 98603486 |
| chrX | 116773411 | 116773461 |
| chrX | 116778083 | 116778133 |
| chr2 | 18251662 | 18251712 |
| chr2 | 18254293 | 18254343 |
| chr3 | 5165886 | 5165936 |
| chr5 | 85027506 | 85027556 |
| chr5 | 125336503 | 125336553 |
| chr6 | 43406107 | 43406157 |
| chr6 | 43413274 | 43413324 |
| chr6 | 80231919 | 80231969 |
| chr6 | 165380628 | 165380678 |
| chr6 | 165387274 | 165387324 |
| chr6 | 165388524 | 165388574 |
| chr8 | 76324035 | 76324085 |
| chr8 | 126958920 | 126958970 |
| chr9 | 41232777 | 41232827 |
| chr12 | 87165 | 87215 |
| chr9 | 133622612 | 133622662 |
| chr9 | 133624416 | 133624466 |
| chr9 | 133626195 | 133626245 |
| chr12 | 3496740 | 3496790 |
| chr12 | 7635369 | 7635419 |
| chr10 | 30855141 | 30855191 |
| chr13 | 109286998 | 109287048 |
| chr10 | 48887289 | 48887339 |
| chr15 | 73072346 | 73072396 |
| chr17 | 31443010 | 31443060 |
| chr17 | 31442768 | 31442818 |

Table 15W. Repeat Category HUERS-P1

| | | |
|---|---|---|
| chr17 | 75396020 | 75396070 |
| chr1 | 1339937 | 1339987 |
| chr1 | 19644411 | 19644461 |
| chr1 | 20311744 | 20311794 |
| chr1 | 91021109 | 91021159 |
| chr1 | 244491072 | 244491122 |
| chr21 | 15101931 | 15101981 |
| chrX | 8859278 | 8859328 |
| chrX | 92036559 | 92036609 |
| chr11 | 10086850 | 10086900 |
| chr11 | 10086677 | 10086727 |
| chr11 | 14162704 | 14162754 |
| chrY | 18474070 | 18474120 |
| chrY | 20510881 | 20510931 |
| chr2 | 36830885 | 36830935 |
| chr2 | 50079611 | 50079661 |
| chr2 | 109334729 | 109334779 |
| chr2 | 134840943 | 134840993 |
| chr2 | 154316635 | 154316685 |
| chr3 | 9649208 | 9649258 |
| chr3 | 9651861 | 9651911 |
| chr3 | 9652843 | 9652893 |
| chr3 | 9654111 | 9654161 |
| chr3 | 9658743 | 9658793 |
| chr3 | 40554525 | 40554575 |
| chr3 | 44465848 | 44465898 |
| chr3 | 44467363 | 44467413 |
| chr3 | 185741472 | 185741522 |
| chr4 | 84477183 | 84477233 |
| chr4 | 101110929 | 101110979 |
| chr5 | 87098704 | 87098754 |
| chr6 | 68115376 | 68115426 |
| chr6 | 147840044 | 147840094 |
| chr11 | 118264175 | 118264225 |
| chr7 | 137120893 | 137120943 |
| chr8 | 91586803 | 91586853 |
| chr8 | 91586500 | 91586550 |
| chr8 | 129070264 | 129070314 |
| chr15 | 67341648 | 67341698 |
| chr15 | 87195445 | 87195495 |
| chr16 | 8890489 | 8890539 |
| chr17 | 259399 | 259449 |

Table 15X. Repeat Category HUERS-P2

| | | |
|---|---|---|
| chr17 | 42472936 | 42472986 |
| chr17 | 43118925 | 43118975 |
| chr1 | 149570165 | 149570215 |
| chr1 | 179724329 | 179724379 |
| chr21 | 22414962 | 22415012 |
| chr21 | 22417515 | 22417565 |
| chr21 | 44762618 | 44762668 |
| chrX | 141948295 | 141948345 |
| chrX | 142111394 | 142111444 |
| chr11 | 26135587 | 26135637 |
| chr2 | 163386958 | 163387008 |
| chr11 | 39953860 | 39953910 |
| chr3 | 97742553 | 97742603 |
| chr3 | 181907134 | 181907184 |
| chr4 | 119212090 | 119212140 |
| chr4 | 119214821 | 119214871 |
| chr6 | 15447666 | 15447716 |
| chr6 | 99745335 | 99745385 |
| chr11 | 115553969 | 115554019 |
| chr7 | 144838835 | 144838885 |
| chr7 | 144841923 | 144841973 |
| chr8 | 111029916 | 111029966 |
| chr9 | 31798136 | 31798186 |
| chr12 | 40982413 | 40982463 |
| chr12 | 73068993 | 73069043 |
| chr13 | 21015943 | 21015993 |
| chr13 | 57231476 | 57231526 |
| chr13 | 60999534 | 60999584 |
| chr13 | 86160799 | 86160849 |
| chr14 | 39184578 | 39184628 |
| chr10 | 54045544 | 54045594 |
| chr10 | 56368379 | 56368429 |

Table 15Y. Repeat Category HUERS-P3

| | | |
|---|---|---|
| chr17 | 52185310 | 52185360 |
| chr17 | 64825485 | 64825535 |
| chr17 | 64826927 | 64826977 |
| chr17 | 73126561 | 73126611 |
| chr17 | 80977296 | 80977346 |
| chr18 | 16910157 | 16910207 |
| chr18 | 29145825 | 29145875 |
| chr18 | 45226069 | 45226119 |
| chr18 | 75871924 | 75871974 |
| chr19 | 23697617 | 23697667 |
| chr19 | 58203562 | 58203612 |
| chr19 | 58203272 | 58203322 |
| chr19 | 59878543 | 59878593 |
| chr19 | 61552513 | 61552563 |
| chr1 | 27132689 | 27132739 |
| chr1 | 32384474 | 32384524 |
| chr1 | 102363651 | 102363701 |
| chr1 | 223656270 | 223656320 |
| chr1 | 242390069 | 242390119 |
| chr1 | 242390126 | 242390176 |
| chr1 | 243201130 | 243201180 |
| chr1 | 243645834 | 243645884 |
| chr20 | 46837506 | 46837556 |
| chr21 | 19976407 | 19976457 |
| chr21 | 36391827 | 36391877 |
| chr22 | 24342784 | 24342834 |
| chrX | 26033665 | 26033715 |
| chrX | 87789407 | 87789457 |
| chrX | 107984908 | 107984958 |
| chrY | 2981340 | 2981390 |
| chrY | 21956934 | 21956984 |
| chr2 | 31890284 | 31890334 |
| chr2 | 108481066 | 108481116 |
| chr11 | 34884517 | 34884567 |
| chr11 | 36658507 | 36658557 |
| chr11 | 40087924 | 40087974 |
| chr2 | 209462598 | 209462648 |
| chr2 | 219417030 | 219417080 |
| chr2 | 219417931 | 219417981 |
| chr2 | 219846914 | 219846964 |
| chr2 | 229375634 | 229375684 |
| chr11 | 59887612 | 59887662 |
| chr3 | 143320860 | 143320910 |
| chr3 | 146297358 | 146297408 |
| chr3 | 173712732 | 173712782 |
| chr3 | 197577793 | 197577843 |
| chr3 | 197900167 | 197900217 |
| chr4 | 36793379 | 36793429 |
| chr4 | 164687848 | 164687898 |
| chr4 | 164689075 | 164689125 |
| chr4 | 164689386 | 164689436 |
| chr5 | 20382016 | 20382066 |
| chr5 | 54518737 | 54518787 |
| chr5 | 80276520 | 80276570 |
| chr5 | 126153252 | 126153302 |
| chr5 | 140864272 | 140864322 |
| chr5 | 142661945 | 142661995 |
| chr11 | 74320868 | 74320918 |
| chr5 | 157127970 | 157128020 |
| chr5 | 180514759 | 180514809 |
| chr6 | 66920360 | 66920410 |
| chr6 | 118946792 | 118946842 |
| chr7 | 8973781 | 8973831 |
| chr7 | 63492843 | 63492893 |
| chr7 | 63495234 | 63495284 |
| chr8 | 34810262 | 34810312 |
| chr11 | 125281717 | 125281767 |
| chr8 | 74993381 | 74993431 |
| chr8 | 88014200 | 88014250 |
| chr9 | 14820611 | 14820661 |
| chr9 | 14821819 | 14821869 |
| chr9 | 15222877 | 15222927 |
| chr9 | 71698633 | 71698683 |
| chr5 | 69006205 | 69006255 |
| chr5 | 69006560 | 69006610 |
| chr5 | 69006678 | 69006728 |
| chr5 | 70521542 | 70521592 |
| chr5 | 70521999 | 70522049 |
| chr5 | 70522244 | 70522294 |
| chr5 | 70522471 | 70522521 |
| chr12 | 7687110 | 7687160 |
| chr10 | 22580498 | 22580548 |
| chr12 | 30888859 | 30888909 |
| chr12 | 103133770 | 103133820 |
| chr12 | 107670719 | 107670769 |
| chr13 | 23368182 | 23368232 |
| chr13 | 29762328 | 29762378 |
| chr13 | 34739212 | 34739262 |
| chr10 | 37618144 | 37618194 |
| chr13 | 58658405 | 58658455 |
| chr13 | 60904274 | 60904324 |
| chr13 | 79890439 | 79890489 |
| chr16 | 29760636 | 29760686 |
| chr17 | 16649836 | 16649886 |

Table 15Z. Repeat Category L1M3f

| | | |
|---|---|---|
| chr10 | 93169362 | 93169412 |
| chr18 | 47332147 | 47332197 |
| chr18 | 58198596 | 58198646 |
| chr18 | 67500685 | 67500735 |
| chr19 | 40082048 | 40082098 |
| chr19 | 41913088 | 41913138 |
| chr19 | 59503551 | 59503601 |
| chr1 | 150109199 | 150109249 |
| chr1 | 157503847 | 157503897 |
| chr1 | 172118239 | 172118289 |
| chr1 | 213541410 | 213541460 |
| chr21 | 43895296 | 43895346 |
| chr22 | 15931991 | 15932041 |
| chrX | 29997181 | 29997231 |
| chrX | 29996942 | 29996992 |
| chrX | 72101369 | 72101419 |
| chrX | 74362894 | 74362944 |
| chrX | 76433936 | 76433986 |
| chrX | 119043458 | 119043508 |
| chrX | 125731983 | 125732033 |
| chrY | 2428455 | 2428505 |
| chr2 | 15106266 | 15106316 |
| chr11 | 20118143 | 20118193 |
| chr11 | 20117954 | 20118004 |
| chr2 | 141796631 | 141796681 |
| chr11 | 34799775 | 34799825 |
| chr11 | 38187209 | 38187259 |
| chr2 | 187849548 | 187849598 |
| chr2 | 195190317 | 195190367 |
| chr2 | 219796563 | 219796613 |
| chr3 | 28178932 | 28178982 |
| chr3 | 175411275 | 175411325 |
| chr4 | 2151462 | 2151512 |
| chr4 | 83317451 | 83317501 |
| chr4 | 90725576 | 90725626 |
| chr4 | 115175721 | 115175771 |
| chr4 | 146489913 | 146489963 |
| chr4 | 165506205 | 165506255 |
| chr5 | 62555177 | 62555227 |
| chr5 | 82359970 | 82360020 |
| chr5 | 93423230 | 93423280 |
| chr5 | 118503048 | 118503098 |
| chr5 | 156258224 | 156258274 |
| chr6 | 29093781 | 29093831 |
| chr11 | 82653958 | 82654008 |
| chr11 | 82657767 | 82657817 |
| chr6 | 70200833 | 70200883 |
| chr6 | 114298275 | 114298325 |
| chr6 | 122546082 | 122546132 |
| chr6 | 127906547 | 127906597 |
| chr11 | 97201180 | 97201230 |
| chr7 | 158146810 | 158146860 |
| chr8 | 6893801 | 6893851 |
| chr8 | 60793858 | 60793908 |
| chr8 | 65023325 | 65023375 |
| chr8 | 104549953 | 104550003 |
| chr9 | 6856383 | 6856433 |
| chr9 | 6857337 | 6857387 |
| chr9 | 35582804 | 35582854 |
| chr9 | 75561423 | 75561473 |
| chr9 | 120734474 | 120734524 |
| chr12 | 3818299 | 3818349 |
| chr12 | 45986388 | 45986438 |
| chr12 | 103819071 | 103819121 |
| chr12 | 108319593 | 108319643 |
| chr12 | 130358782 | 130358832 |
| chr13 | 86917615 | 86917665 |
| chr14 | 32982246 | 32982296 |
| chr10 | 48138279 | 48138329 |
| chr15 | 38498286 | 38498336 |
| chr15 | 59697282 | 59697332 |
| chr15 | 59904050 | 59904100 |
| chr16 | 3388760 | 3388810 |
| chr16 | 75589928 | 75589978 |
| chr10 | 75465252 | 75465302 |
| chr17 | 21214505 | 21214555 |
| chr17 | 21214305 | 21214355 |

Table 15AA. Repeat Category L1MA1

| | | |
|---|---|---|
| chr10 | 81771076 | 81771126 |
| chr10 | 81770669 | 81770719 |
| chr10 | 83613460 | 83613510 |
| chr17 | 48087778 | 48087828 |
| chr17 | 52098872 | 52098922 |
| chr17 | 59590004 | 59590054 |
| chr17 | 59794974 | 59795024 |
| chr17 | 67420693 | 67420743 |
| chr17 | 76489363 | 76489413 |
| chr18 | 7429723 | 7429773 |
| chr18 | 11638686 | 11638736 |
| chr18 | 13054250 | 13054300 |
| chr18 | 25000673 | 25000723 |
| chr10 | 96255336 | 96255386 |
| chr18 | 33901594 | 33901644 |
| chr18 | 58591582 | 58591632 |
| chr18 | 64152121 | 64152171 |
| chr18 | 72539223 | 72539273 |
| chr18 | 72539067 | 72539117 |
| chr19 | 2045674 | 2045724 |
| chr19 | 8208827 | 8208877 |
| chr19 | 9776521 | 9776571 |
| chr19 | 12169020 | 12169070 |
| chr19 | 17066286 | 17066336 |
| chr10 | 103637241 | 103637291 |
| chr19 | 19106662 | 19106712 |
| chr19 | 33900734 | 33900784 |
| chr19 | 44043498 | 44043548 |
| chr19 | 50167812 | 50167862 |
| chr19 | 50604758 | 50604808 |
| chr19 | 50605703 | 50605753 |
| chr19 | 51766075 | 51766125 |
| chr10 | 111878083 | 111878133 |
| chr19 | 54204947 | 54204997 |
| chr19 | 58441445 | 58441495 |
| chr19 | 59600853 | 59600903 |
| chr19 | 59684475 | 59684525 |
| chr19 | 59742363 | 59742413 |
| chr19 | 61380295 | 61380345 |
| chr19 | 62996597 | 62996647 |
| chr19 | 63072622 | 63072672 |
| chr19 | 63127040 | 63127090 |
| chr19 | 63212625 | 63212675 |
| chr1 | 8139853 | 8139903 |
| chr1 | 11137594 | 11137644 |
| chr1 | 11884710 | 11884760 |
| chr1 | 14692310 | 14692360 |
| chr1 | 18822641 | 18822691 |
| chr1 | 28500574 | 28500624 |
| chr1 | 30085553 | 30085603 |
| chr1 | 33181608 | 33181658 |
| chr1 | 42096532 | 42096582 |
| chr1 | 49847847 | 49847897 |
| chr1 | 54687981 | 54688031 |
| chr1 | 63596268 | 63596318 |
| chr1 | 71546664 | 71546714 |
| chr1 | 81080704 | 81080754 |
| chr1 | 81550129 | 81550179 |
| chr10 | 6707061 | 6707111 |
| chr10 | 125618608 | 125618658 |
| chr1 | 100454553 | 100454603 |
| chr1 | 100673040 | 100673090 |
| chr1 | 100845210 | 100845260 |
| chr1 | 104263309 | 104263359 |
| chr1 | 116751098 | 116751148 |
| chr1 | 140642544 | 140642594 |
| chr1 | 141130625 | 141130675 |
| chr1 | 149101991 | 149102041 |
| chr1 | 154206725 | 154206775 |
| chr1 | 154931370 | 154931420 |
| chr1 | 155009110 | 155009160 |
| chr1 | 161292981 | 161293031 |
| chr1 | 161513309 | 161513359 |
| chr1 | 165111513 | 165111563 |
| chr1 | 168252976 | 168253026 |
| chr1 | 171490144 | 171490194 |
| chr1 | 173606513 | 173606563 |
| chr1 | 177918084 | 177918134 |
| chr1 | 178276685 | 178276735 |
| chr1 | 180188165 | 180188215 |
| chr1 | 181519142 | 181519192 |
| chr1 | 184425956 | 184426006 |
| chr1 | 189991703 | 189991753 |
| chr1 | 191908143 | 191908193 |
| chr1 | 192125819 | 192125869 |
| chr1 | 194145689 | 194145739 |
| chr1 | 199787537 | 199787587 |
| chr1 | 199787212 | 199787262 |
| chr1 | 200139108 | 200139158 |
| chr1 | 202968721 | 202968771 |
| chr1 | 210376216 | 210376266 |
| chr1 | 210383109 | 210383159 |
| chr1 | 216138589 | 216138639 |
| chr1 | 221607376 | 221607426 |
| chr1 | 238204433 | 238204483 |
| chr1 | 241978987 | 241979037 |
| chr1 | 242244752 | 242244802 |
| chr1 | 244656223 | 244656273 |
| chr1 | 244717207 | 244717257 |
| chr10 | 133241280 | 133241330 |
| chr20 | 21863251 | 21863301 |
| chr20 | 41038151 | 41038201 |
| chr20 | 44042453 | 44042503 |
| chr20 | 44919808 | 44919858 |
| chr20 | 46802945 | 46802995 |
| chr10 | 134099395 | 134099445 |
| chr20 | 52784186 | 52784236 |
| chr20 | 54793262 | 54793312 |
| chr20 | 56644484 | 56644534 |
| chr20 | 61644189 | 61644239 |
| chr21 | 30796226 | 30796276 |
| chr21 | 33680614 | 33680664 |
| chr21 | 35042487 | 35042537 |
| chr22 | 19406704 | 19406754 |
| chr22 | 19406770 | 19406820 |
| chr22 | 20610320 | 20610370 |
| chr22 | 20766708 | 20766758 |
| chr22 | 23817293 | 23817343 |
| chr22 | 27160284 | 27160334 |
| chr22 | 28549951 | 28550001 |
| chr22 | 32451827 | 32451877 |
| chr22 | 33261197 | 33261247 |
| chr11 | 2067037 | 2067087 |
| chrX | 8056752 | 8056802 |
| chr11 | 5462868 | 5462918 |
| chrX | 22928822 | 22928872 |
| chrX | 23606381 | 23606431 |
| chrX | 31140711 | 31140761 |
| chrX | 33173422 | 33173472 |
| chrX | 33187273 | 33187323 |
| chrX | 33213000 | 33213050 |
| chrX | 36931109 | 36931159 |
| chrX | 40529545 | 40529595 |
| chrX | 47003616 | 47003666 |
| chrX | 53583673 | 53583723 |
| chrX | 63146131 | 63146181 |
| chrX | 72734940 | 72734990 |
| chrX | 76247630 | 76247680 |
| chrX | 76598061 | 76598111 |
| chrX | 77344119 | 77344169 |
| chrX | 77681120 | 77681170 |
| chrX | 82449928 | 82449978 |
| chrX | 82573468 | 82573518 |
| chrX | 84313364 | 84313414 |
| chrX | 84322641 | 84322691 |
| chrX | 85909004 | 85909054 |
| chrX | 92957723 | 92957773 |
| chrX | 94091581 | 94091631 |
| chrX | 99419975 | 99420025 |
| chrX | 100309146 | 100309196 |
| chrX | 100431512 | 100431562 |
| chrX | 111596830 | 111596880 |
| chr11 | 10232050 | 10232100 |
| chrX | 115246208 | 115246258 |
| chrX | 118482309 | 118482359 |
| chrX | 120362724 | 120362774 |
| chrX | 124762385 | 124762435 |
| chrX | 125018885 | 125018935 |
| chrX | 130735538 | 130735588 |
| chrX | 133945371 | 133945421 |
| chrX | 133945037 | 133945087 |
| chrX | 134309482 | 134309532 |
| chr11 | 12235972 | 12236022 |
| chrX | 139222855 | 139222905 |
| chrX | 141311946 | 141311996 |
| chrX | 141576135 | 141576185 |
| chrX | 142866165 | 142866215 |
| chrX | 145492726 | 145492776 |
| chrY | 15190110 | 15190160 |
| chrY | 21762252 | 21762302 |
| chr2 | 14982695 | 14982745 |
| chr2 | 24121271 | 24121321 |
| chr2 | 26469692 | 26469742 |
| chr2 | 27788568 | 27788618 |
| chr2 | 30636170 | 30636220 |
| chr2 | 34879554 | 34879604 |
| chr2 | 35057830 | 35057880 |
| chr11 | 27840968 | 27841018 |
| chr2 | 95013707 | 95013757 |
| chr2 | 96527170 | 96527220 |
| chr11 | 30599869 | 30599919 |
| chr2 | 103769162 | 103769212 |
| chr2 | 104577650 | 104577700 |
| chr2 | 105960587 | 105960637 |
| chr11 | 31964446 | 31964496 |
| chr2 | 113394579 | 113394629 |
| chr2 | 117429976 | 117430026 |
| chr2 | 124804138 | 124804188 |
| chr2 | 127857596 | 127857646 |
| chr2 | 135881211 | 135881261 |
| chr2 | 136996542 | 136996592 |
| chr11 | 33995641 | 33995691 |
| chr2 | 139603289 | 139603339 |
| chr2 | 141690133 | 141690183 |
| chr2 | 170625012 | 170625062 |
| chr2 | 188590323 | 188590373 |
| chr11 | 38767697 | 38767747 |
| chr2 | 194846998 | 194847048 |
| chr2 | 200129822 | 200129872 |
| chr11 | 40368467 | 40368517 |
| chr11 | 42120354 | 42120404 |
| chr2 | 215589753 | 215589803 |
| chr11 | 43688772 | 43688822 |
| chr2 | 226331579 | 226331629 |
| chr2 | 232254670 | 232254720 |
| chr2 | 236485370 | 236485420 |
| chr2 | 242658365 | 242658415 |
| chr2 | 243290609 | 243290659 |
| chr3 | 4684919 | 4684969 |
| chr11 | 47002531 | 47002581 |
| chr3 | 26797675 | 26797725 |
| chr3 | 30779548 | 30779598 |
| chr3 | 33781374 | 33781424 |
| chr3 | 34687998 | 34688048 |
| chr11 | 49961912 | 49961962 |
| chr3 | 49580613 | 49580663 |
| chr3 | 49581959 | 49582009 |
| chr3 | 50768729 | 50768779 |
| chr3 | 56400291 | 56400341 |
| chr3 | 67131081 | 67131131 |
| chr3 | 73209812 | 73209862 |
| chr3 | 79757795 | 79757845 |
| chr3 | 84921649 | 84921699 |
| chr3 | 86668620 | 86668670 |
| chr3 | 87655079 | 87655129 |
| chr3 | 103503475 | 103503525 |
| chr11 | 59596795 | 59596845 |
| chr3 | 110662434 | 110662484 |
| chr3 | 111497749 | 111497799 |
| chr11 | 60127596 | 60127646 |
| chr3 | 119431532 | 119431582 |
| chr3 | 124826133 | 124826183 |
| chr3 | 138911732 | 138911782 |
| chr3 | 146083846 | 146083896 |
| chr3 | 156507106 | 156507156 |
| chr3 | 162982713 | 162982763 |
| chr3 | 164115921 | 164115971 |
| chr3 | 164342029 | 164342079 |
| chr3 | 164341916 | 164341966 |
| chr3 | 164505142 | 164505192 |
| chr3 | 167293881 | 167293931 |
| chr3 | 167849583 | 167849633 |
| chr3 | 170380072 | 170380122 |
| chr3 | 173610009 | 173610059 |
| chr3 | 175936327 | 175936377 |
| chr3 | 179655695 | 179655745 |
| chr3 | 180330420 | 180330470 |
| chr3 | 181301446 | 181301496 |
| chr3 | 187599679 | 187599729 |
| chr3 | 192075885 | 192075935 |
| chr3 | 197645829 | 197645879 |
| chr4 | 8639258 | 8639308 |
| chr4 | 10237446 | 10237496 |
| chr4 | 20093016 | 20093066 |
| chr4 | 23560473 | 23560523 |
| chr4 | 34695621 | 34695671 |
| chr4 | 77147238 | 77147288 |
| chr4 | 102133275 | 102133325 |
| chr4 | 110056319 | 110056369 |
| chr4 | 112512759 | 112512809 |
| chr4 | 112518068 | 112518118 |
| chr4 | 116657231 | 116657281 |
| chr4 | 123901302 | 123901352 |
| chr4 | 124132784 | 124132834 |
| chr4 | 156341146 | 156341196 |
| chr4 | 160004500 | 160004550 |
| chr4 | 160929009 | 160929059 |
| chr4 | 171790517 | 171790567 |
| chr4 | 189502026 | 189502076 |
| chr5 | 28721989 | 28722039 |
| chr5 | 31723478 | 31723528 |
| chr5 | 37565317 | 37565367 |
| chr5 | 41365724 | 41365774 |
| chr5 | 43518386 | 43518436 |
| chr5 | 50575668 | 50575718 |
| chr11 | 70145602 | 70145652 |
| chr5 | 56220535 | 56220585 |
| chr5 | 58180120 | 58180170 |
| chr5 | 62720865 | 62720915 |
| chr5 | 63504070 | 63504120 |
| chr5 | 66799360 | 66799410 |
| chr5 | 80164167 | 80164217 |
| chr5 | 93574460 | 93574510 |
| chr5 | 99702805 | 99702855 |
| chr5 | 104778613 | 104778663 |
| chr5 | 109688437 | 109688487 |
| chr5 | 118620407 | 118620457 |
| chr5 | 130822727 | 130822777 |
| chr5 | 133149849 | 133149899 |
| chr5 | 144040757 | 144040807 |
| chr5 | 151961785 | 151961835 |
| chr5 | 154512279 | 154512329 |
| chr5 | 178194743 | 178194793 |
| chr5 | 178771930 | 178771980 |
| chr5 | 179913231 | 179913281 |
| chr5 | 180453373 | 180453423 |
| chr6 | 13167934 | 13167984 |
| chr6 | 15420709 | 15420759 |
| chr10 | 15088803 | 15088853 |
| chr6 | 28813592 | 28813642 |
| chr6 | 30313779 | 30313829 |
| chr6 | 30827964 | 30828014 |
| chr6 | 31054581 | 31054631 |
| chr6 | 37206577 | 37206627 |
| chr6 | 41639559 | 41639609 |
| chr6 | 42487923 | 42487973 |
| chr6 | 78849435 | 78849485 |
| chr6 | 79593045 | 79593095 |
| chr6 | 81701502 | 81701552 |
| chr6 | 82710600 | 82710650 |
| chr6 | 85879054 | 85879104 |
| chr6 | 93312200 | 93312250 |
| chr6 | 101833145 | 101833195 |
| chr6 | 103132727 | 103132777 |
| chr11 | 93925178 | 93925228 |
| chr6 | 111282857 | 111282907 |
| chr6 | 113887705 | 113887755 |
| chr6 | 114390247 | 114390297 |
| chr6 | 117793448 | 117793498 |
| chr6 | 119352831 | 119352881 |
| chr6 | 130960654 | 130960704 |
| chr6 | 142350076 | 142350126 |
| chr11 | 98747850 | 98747900 |
| chr6 | 147588496 | 147588546 |
| chr6 | 162891427 | 162891477 |
| chr6 | 162893180 | 162893230 |
| chr11 | 104895418 | 104895468 |
| chr7 | 648581 | 648631 |
| chr7 | 2146513 | 2146563 |
| chr7 | 2555383 | 2555433 |
| chr11 | 108669281 | 108669331 |
| chr7 | 7890931 | 7890981 |
| chr7 | 13376499 | 13376549 |
| chr7 | 14321680 | 14321730 |
| chr7 | 24291064 | 24291114 |
| chr7 | 39628403 | 39628453 |
| chr7 | 47087542 | 47087592 |
| chr7 | 73028625 | 73028675 |
| chr7 | 78938001 | 78938051 |
| chr7 | 80041549 | 80041599 |
| chr7 | 89010471 | 89010521 |
| chr7 | 94810707 | 94810757 |
| chr7 | 96828040 | 96828090 |
| chr7 | 108340480 | 108340530 |
| chr7 | 111898941 | 111898991 |
| chr11 | 118004117 | 118004167 |
| chr7 | 124487947 | 124487997 |
| chr7 | 125026739 | 125026789 |
| chr7 | 128278711 | 128278761 |
| chr7 | 131630987 | 131631037 |
| chr7 | 145647887 | 145647937 |
| chr7 | 154334050 | 154334100 |
| chr7 | 156011917 | 156011967 |
| chr8 | 3531291 | 3531341 |
| chr8 | 8203962 | 8204012 |
| chr8 | 14014781 | 14014831 |
| chr8 | 19643861 | 19643911 |
| chr8 | 20415928 | 20415978 |
| chr8 | 23689304 | 23689354 |
| chr8 | 28182159 | 28182209 |
| chr8 | 55017659 | 55017709 |
| chr8 | 59601959 | 59602009 |
| chr8 | 92669081 | 92669131 |
| chr8 | 100904235 | 100904285 |
| chr8 | 107745747 | 107745797 |
| chr8 | 120521578 | 120521628 |
| chr10 | 19196079 | 19196129 |
| chr8 | 143050292 | 143050342 |
| chr10 | 19526060 | 19526110 |
| chr11 | 130972445 | 130972495 |
| chr8 | 146222648 | 146222698 |
| chr9 | 4065122 | 4065172 |
| chr9 | 8470826 | 8470876 |
| chr9 | 12683319 | 12683369 |
| chr9 | 17542337 | 17542387 |
| chr9 | 22285815 | 22285865 |
| chr9 | 22855678 | 22855728 |
| chr9 | 25934001 | 25934051 |
| chr9 | 26473423 | 26473473 |
| chr9 | 26475580 | 26475630 |
| chr9 | 26482072 | 26482122 |
| chr9 | 30089964 | 30090014 |
| chr9 | 32683725 | 32683775 |
| chr9 | 34233563 | 34233613 |
| chr9 | 37627054 | 37627104 |
| chr9 | 37627280 | 37627330 |
| chr9 | 85553088 | 85553138 |
| chr9 | 92626918 | 92626968 |
| chr9 | 109987279 | 109987329 |
| chr9 | 109987957 | 109988007 |
| chr9 | 111756562 | 111756612 |
| chr9 | 114733933 | 114733983 |
| chr9 | 123931303 | 123931353 |
| chr9 | 133745564 | 133745614 |
| chr12 | 3713765 | 3713815 |
| chr12 | 10386936 | 10386986 |
| chr12 | 23364428 | 23364478 |
| chr12 | 26218643 | 26218693 |
| chr12 | 27045054 | 27045104 |
| chr12 | 30457769 | 30457819 |
| chr12 | 40829830 | 40829880 |
| chr10 | 26553600 | 26553650 |
| chr10 | 26684662 | 26684712 |
| chr12 | 64810982 | 64811032 |
| chr12 | 69671475 | 69671525 |
| chr12 | 89779197 | 89779247 |
| chr12 | 105734164 | 105734214 |
| chr12 | 110839619 | 110839669 |
| chr12 | 118270791 | 118270841 |
| chr12 | 118270650 | 118270700 |
| chr12 | 118875135 | 118875185 |
| chr12 | 119151244 | 119151294 |
| chr12 | 119796065 | 119796115 |
| chr12 | 122521829 | 122521879 |
| chr10 | 34005051 | 34005101 |
| chr12 | 126747577 | 126747627 |
| chr10 | 34472928 | 34472978 |
| chr12 | 130103030 | 130103080 |
| chr13 | 18592994 | 18593044 |
| chr13 | 22063792 | 22063842 |
| chr13 | 31801892 | 31801942 |
| chr13 | 39318178 | 39318228 |
| chr13 | 43811287 | 43811337 |
| chr13 | 48139639 | 48139689 |
| chr13 | 60702285 | 60702335 |
| chr13 | 75704504 | 75704554 |
| chr13 | 79863010 | 79863060 |
| chr13 | 81405630 | 81405680 |
| chr13 | 85290690 | 85290740 |
| chr13 | 87612605 | 87612655 |
| chr13 | 93368175 | 93368225 |
| chr13 | 96500562 | 96500612 |
| chr13 | 99609967 | 99610017 |
| chr14 | 18795614 | 18795664 |
| chr14 | 18828107 | 18828157 |
| chr10 | 45347803 | 45347853 |
| chr14 | 24465441 | 24465491 |
| chr14 | 24995530 | 24995580 |
| chr14 | 33386668 | 33386718 |
| chr14 | 39634501 | 39634551 |
| chr14 | 49139244 | 49139294 |
| chr14 | 50147499 | 50147549 |
| chr14 | 62697354 | 62697404 |
| chr14 | 68567876 | 68567926 |
| chr14 | 78741830 | 78741880 |
| chr14 | 82555168 | 82555218 |
| chr14 | 86971134 | 86971184 |
| chr10 | 54505368 | 54505418 |
| chr14 | 90279011 | 90279061 |
| chr14 | 93943739 | 93943789 |
| chr14 | 94308759 | 94308809 |
| chr14 | 98966281 | 98966331 |
| chr10 | 58144743 | 58144793 |
| chr14 | 100259728 | 100259778 |
| chr14 | 101844714 | 101844764 |
| chr14 | 103775281 | 103775331 |
| chr15 | 20836042 | 20836092 |
| chr15 | 25716038 | 25716088 |
| chr15 | 31276822 | 31276872 |
| chr15 | 37575128 | 37575178 |
| chr10 | 66195734 | 66195784 |
| chr15 | 62779301 | 62779351 |
| chr15 | 63801566 | 63801616 |
| chr15 | 69707299 | 69707349 |
| chr15 | 92217667 | 92217717 |
| chr16 | 1006769 | 1006819 |
| chr16 | 5015635 | 5015685 |
| chr16 | 5668544 | 5668594 |
| chr16 | 12807997 | 12808047 |
| chr16 | 14620157 | 14620207 |
| chr16 | 17386720 | 17386770 |
| chr16 | 17386661 | 17386711 |
| chr16 | 22351730 | 22351780 |
| chr16 | 24868735 | 24868785 |
| chr16 | 26821519 | 26821569 |
| chr16 | 26822711 | 26822761 |
| chr16 | 31584958 | 31585008 |
| chr16 | 59135564 | 59135614 |
| chr16 | 60377051 | 60377101 |
| chr16 | 60476341 | 60476391 |
| chr16 | 61650638 | 61650688 |
| chr16 | 68539388 | 68539438 |
| chr16 | 70029738 | 70029788 |
| chr10 | 74418490 | 74418540 |
| chr16 | 87388740 | 87388790 |
| chr16 | 89083325 | 89083375 |
| chr17 | 3941560 | 3941610 |
| chr17 | 4417922 | 4417972 |
| chr17 | 4915129 | 4915179 |
| chr17 | 5358342 | 5358392 |
| chr17 | 8083468 | 8083518 |
| chr17 | 8896882 | 8896932 |
| chr17 | 10793014 | 10793064 |
| chr17 | 12177580 | 12177630 |

Table 15AB. Repeat Category L1MA7

| | | |
|---|---|---|
| chr10 | 83209575 | 83209625 |
| chr10 | 83210059 | 83210109 |
| chr17 | 46649462 | 46649512 |
| chr17 | 51777550 | 51777600 |
| chr18 | 4312645 | 4312695 |
| chr18 | 10108922 | 10108972 |
| chr18 | 11668056 | 11668106 |
| chr18 | 27566828 | 27566878 |
| chr10 | 95939590 | 95939640 |
| chr18 | 31813101 | 31813151 |
| chr18 | 32635201 | 32635251 |
| chr18 | 46656179 | 46656229 |
| chr18 | 48530558 | 48530608 |
| chr18 | 63934048 | 63934098 |
| chr18 | 65696759 | 65696809 |
| chr18 | 71907355 | 71907405 |
| chr18 | 73572363 | 73572413 |
| chr19 | 6942096 | 6942146 |
| chr19 | 7141659 | 7141709 |
| chr19 | 9000313 | 9000363 |
| chr19 | 9001692 | 9001742 |
| chr19 | 9039972 | 9040022 |
| chr19 | 15854112 | 15854162 |
| chr19 | 22071291 | 22071341 |
| chr19 | 22849766 | 22849816 |
| chr19 | 23688049 | 23688099 |
| chr19 | 38038125 | 38038175 |
| chr19 | 38039254 | 38039304 |
| chr19 | 39589522 | 39589572 |
| chr19 | 41682823 | 41682873 |
| chr19 | 41862072 | 41862122 |
| chr19 | 42962384 | 42962434 |
| chr10 | 106170753 | 106170803 |
| chr19 | 53230284 | 53230334 |
| chr10 | 113623780 | 113623830 |
| chr19 | 61103560 | 61103610 |
| chr19 | 62199771 | 62199821 |
| chr10 | 116792719 | 116792769 |
| chr10 | 116813287 | 116813337 |
| chr1 | 22201630 | 22201680 |
| chr10 | 120273024 | 120273074 |
| chr1 | 29013079 | 29013129 |
| chr1 | 34475519 | 34475569 |
| chr1 | 35060150 | 35060200 |
| chr1 | 45296971 | 45297021 |
| chr1 | 47104738 | 47104788 |
| chr1 | 50527762 | 50527812 |
| chr1 | 66262007 | 66262057 |
| chr1 | 79471241 | 79471291 |
| chr1 | 119131780 | 119131830 |
| chr1 | 149781473 | 149781523 |
| chr1 | 171380149 | 171380199 |
| chr1 | 187841372 | 187841422 |
| chr1 | 192221205 | 192221255 |
| chr1 | 193550108 | 193550158 |
| chr1 | 201023797 | 201023847 |
| chr1 | 201077789 | 201077839 |
| chr10 | 131684610 | 131684660 |
| chr1 | 239576606 | 239576656 |
| chr1 | 244364811 | 244364861 |
| chr20 | 242513 | 242563 |
| chr10 | 132809618 | 132809668 |
| chr20 | 8486434 | 8486484 |
| chr20 | 17781090 | 17781140 |
| chr20 | 19450657 | 19450707 |
| chr20 | 43352778 | 43352828 |
| chr20 | 44885677 | 44885727 |
| chr21 | 15106539 | 15106589 |
| chr21 | 27715117 | 27715167 |
| chr21 | 28839196 | 28839246 |
| chr21 | 44208327 | 44208377 |
| chr21 | 46825927 | 46825977 |
| chr21 | 46825866 | 46825916 |
| chr22 | 22157976 | 22158026 |
| chr22 | 27326915 | 27326965 |
| chr22 | 30234033 | 30234083 |
| chr22 | 34712128 | 34712178 |
| chr22 | 37616452 | 37616502 |
| chr22 | 46242515 | 46242565 |
| chrX | 13623647 | 13623697 |
| chrX | 13623489 | 13623539 |
| chrX | 14732970 | 14733020 |
| chrX | 23046420 | 23046470 |
| chrX | 27276479 | 27276529 |
| chrX | 27277838 | 27277888 |
| chrX | 28387997 | 28388047 |
| chrX | 39627461 | 39627511 |
| chrX | 43899101 | 43899151 |
| chrX | 55277557 | 55277607 |
| chrX | 64239574 | 64239624 |
| chrX | 70306563 | 70306613 |
| chrX | 71107436 | 71107486 |
| chrX | 71109494 | 71109544 |
| chrX | 72058539 | 72058589 |
| chrX | 72910886 | 72910936 |
| chrX | 75477266 | 75477316 |
| chrX | 76143638 | 76143688 |
| chrX | 91624098 | 91624148 |
| chrX | 99245274 | 99245324 |
| chrX | 99322824 | 99322874 |
| chrX | 102047218 | 102047268 |
| chrX | 109708267 | 109708317 |
| chrX | 120220776 | 120220826 |
| chrX | 126222544 | 126222594 |
| chrX | 128106379 | 128106429 |
| chr11 | 11752635 | 11752685 |
| chrX | 146622357 | 146622407 |
| chrX | 148641559 | 148641609 |
| chrY | 2820953 | 2821003 |
| chrY | 6443553 | 6443603 |
| chrY | 8864325 | 8864375 |
| chrY | 16019709 | 16019759 |
| chrY | 22275330 | 22275380 |
| chr2 | 1960852 | 1960902 |
| chr2 | 35012377 | 35012427 |
| chr2 | 39682714 | 39682764 |
| chr2 | 48547553 | 48547603 |
| chr2 | 57665488 | 57665538 |
| chr2 | 63468385 | 63468435 |
| chr2 | 67086874 | 67086924 |
| chr11 | 24949967 | 24950017 |
| chr2 | 82036248 | 82036298 |
| chr11 | 29710195 | 29710245 |
| chr2 | 99264620 | 99264670 |
| chr2 | 102451010 | 102451060 |
| chr2 | 145877403 | 145877453 |
| chr2 | 148476654 | 148476704 |
| chr2 | 160280025 | 160280075 |
| chr2 | 170683926 | 170683976 |
| chr2 | 173341015 | 173341065 |
| chr2 | 184306394 | 184306444 |
| chr2 | 184402499 | 184402549 |
| chr2 | 186096695 | 186096745 |
| chr2 | 187178594 | 187178644 |
| chr2 | 191200693 | 191200743 |
| chr2 | 201276033 | 201276083 |
| chr11 | 41618493 | 41618543 |
| chr2 | 221207418 | 221207468 |
| chr3 | 4475585 | 4475635 |
| chr3 | 11925816 | 11925866 |
| chr3 | 11925621 | 11925671 |
| chr3 | 11930169 | 11930219 |
| chr3 | 24592763 | 24592813 |
| chr3 | 26111616 | 26111666 |
| chr3 | 32860390 | 32860440 |
| chr3 | 41503896 | 41503946 |
| chr3 | 46560705 | 46560755 |
| chr3 | 46953355 | 46953405 |
| chr3 | 51226858 | 51226908 |
| chr3 | 51291229 | 51291279 |
| chr3 | 87753212 | 87753262 |
| chr3 | 130497960 | 130498010 |
| chr3 | 130958398 | 130958448 |
| chr3 | 130958217 | 130958267 |
| chr3 | 139566847 | 139566897 |
| chr3 | 176229362 | 176229412 |
| chr3 | 188126441 | 188126491 |
| chr3 | 188129108 | 188129158 |
| chr4 | 635963 | 636013 |
| chr4 | 3323607 | 3323657 |
| chr4 | 4142127 | 4142177 |
| chr4 | 4968519 | 4968569 |
| chr4 | 9270669 | 9270719 |
| chr4 | 14745418 | 14745468 |
| chr4 | 14745574 | 14745624 |
| chr4 | 31172222 | 31172272 |
| chr4 | 37276957 | 37277007 |
| chr4 | 76859476 | 76859526 |
| chr4 | 83336949 | 83336999 |
| chr4 | 85555334 | 85555384 |
| chr4 | 89833227 | 89833277 |
| chr4 | 92309832 | 92309882 |
| chr4 | 108137125 | 108137175 |
| chr11 | 66342557 | 66342607 |
| chr4 | 137263406 | 137263456 |
| chr4 | 164293879 | 164293929 |
| chr4 | 184661976 | 184662026 |
| chr4 | 184663704 | 184663754 |
| chr4 | 191339686 | 191339736 |
| chr4 | 191339781 | 191339831 |
| chr4 | 191340027 | 191340077 |
| chr4 | 191340267 | 191340317 |
| chr5 | 3732704 | 3732754 |
| chr5 | 13890872 | 13890922 |
| chr5 | 28580990 | 28581040 |
| chr5 | 33554859 | 33554909 |
| chr5 | 41601983 | 41602033 |
| chr5 | 57967780 | 57967830 |
| chr5 | 81109717 | 81109767 |
| chr5 | 87136721 | 87136771 |
| chr5 | 92040951 | 92041001 |
| chr5 | 92042472 | 92042522 |
| chr11 | 71308715 | 71308765 |
| chr5 | 105471321 | 105471371 |
| chr5 | 123331168 | 123331218 |
| chr5 | 125064102 | 125064152 |
| chr5 | 125494342 | 125494392 |
| chr5 | 125497064 | 125497114 |
| chr5 | 130167242 | 130167292 |
| chr5 | 134136097 | 134136147 |
| chr11 | 73367780 | 73367830 |
| chr5 | 148392299 | 148392349 |
| chr5 | 154391654 | 154391704 |
| chr5 | 160412711 | 160412761 |
| chr5 | 171695755 | 171695805 |
| chr5 | 180259665 | 180259715 |
| chr5 | 180502746 | 180502796 |
| chr11 | 77402115 | 77402165 |
| chr6 | 17494150 | 17494200 |
| chr6 | 21409903 | 21409953 |
| chr6 | 24142989 | 24143039 |
| chr6 | 27609680 | 27609730 |
| chr6 | 30613708 | 30613758 |
| chr6 | 31903847 | 31903897 |
| chr6 | 32621369 | 32621419 |
| chr6 | 32670388 | 32670438 |
| chr6 | 32670511 | 32670561 |
| chr6 | 34278963 | 34279013 |
| chr6 | 44040799 | 44040849 |
| chr6 | 48384900 | 48384950 |
| chr6 | 71154337 | 71154387 |
| chr6 | 72350694 | 72350744 |
| chr6 | 74477510 | 74477560 |
| chr6 | 74494538 | 74494588 |
| chr6 | 88231900 | 88231950 |
| chr6 | 94191771 | 94191821 |
| chr6 | 95647283 | 95647333 |
| chr6 | 95654438 | 95654488 |
| chr6 | 103305087 | 103305137 |
| chr6 | 108683589 | 108683639 |
| chr6 | 111786902 | 111786952 |
| chr6 | 119927959 | 119928009 |
| chr6 | 123212321 | 123212371 |
| chr6 | 150195012 | 150195062 |
| chr6 | 158318218 | 158318268 |
| chr6 | 161169488 | 161169538 |
| chr6 | 161169553 | 161169603 |
| chr6 | 161169907 | 161169957 |
| chr6 | 161438725 | 161438775 |
| chr6 | 161439080 | 161439130 |
| chr6 | 161439198 | 161439248 |
| chr6 | 164294186 | 164294236 |
| chr6 | 164294328 | 164294378 |
| chr6 | 168130458 | 168130508 |
| chr6 | 170311683 | 170311733 |
| chr11 | 107015524 | 107015574 |
| chr7 | 1556009 | 1556059 |
| chr7 | 2653220 | 2653270 |
| chr7 | 3979398 | 3979448 |
| chr7 | 13233606 | 13233656 |
| chr7 | 14062802 | 14062852 |
| chr7 | 22420423 | 22420473 |
| chr7 | 28688303 | 28688353 |
| chr7 | 37218507 | 37218557 |
| chr7 | 39741862 | 39741912 |
| chr11 | 112863406 | 112863456 |
| chr7 | 55655177 | 55655227 |
| chr7 | 55709561 | 55709611 |
| chr7 | 63725392 | 63725442 |
| chr7 | 63725445 | 63725495 |
| chr7 | 65802600 | 65802650 |
| chr7 | 67950775 | 67950825 |
| chr7 | 80050425 | 80050475 |
| chr7 | 96487577 | 96487627 |
| chr7 | 107103857 | 107103907 |
| chr7 | 136282292 | 136282342 |
| chr7 | 145647887 | 145647937 |
| chr7 | 151831618 | 151831668 |
| chr7 | 155752028 | 155752078 |
| chr8 | 834907 | 834957 |
| chr8 | 2033156 | 2033206 |
| chr8 | 5349139 | 5349189 |
| chr8 | 6903315 | 6903365 |
| chr8 | 6903935 | 6903985 |
| chr8 | 11951100 | 11951150 |
| chr8 | 12437456 | 12437506 |
| chr8 | 12436680 | 12436730 |
| chr8 | 12876889 | 12876939 |
| chr11 | 122445321 | 122445371 |
| chr8 | 16908112 | 16908162 |
| chr8 | 17232192 | 17232242 |
| chr8 | 38975406 | 38975456 |
| chr8 | 57522335 | 57522385 |
| chr8 | 57521994 | 57522044 |
| chr8 | 65836640 | 65836690 |
| chr8 | 66591579 | 66591629 |
| chr8 | 74390950 | 74391000 |
| chr8 | 89491775 | 89491825 |
| chr8 | 90279395 | 90279445 |
| chr8 | 90336668 | 90336718 |
| chr8 | 91023089 | 91023139 |
| chr8 | 97118687 | 97118737 |
| chr8 | 97622262 | 97622312 |
| chr8 | 99211113 | 99211163 |
| chr8 | 103101496 | 103101546 |
| chr8 | 132336492 | 132336542 |
| chr8 | 141300107 | 141300157 |
| chr11 | 130572650 | 130572700 |
| chr9 | 1127264 | 1127314 |
| chr9 | 4947610 | 4947660 |
| chr9 | 6678577 | 6678627 |
| chr9 | 9148201 | 9148251 |
| chr9 | 15870108 | 15870158 |
| chr9 | 21815308 | 21815358 |
| chr9 | 29649846 | 29649896 |
| chr9 | 38468265 | 38468315 |
| chr9 | 66957699 | 66957749 |
| chr9 | 72670216 | 72670266 |
| chr9 | 84636915 | 84636965 |
| chr9 | 86339384 | 86339434 |
| chr9 | 89335347 | 89335397 |
| chr9 | 92609153 | 92609203 |
| chr9 | 93826033 | 93826083 |
| chr9 | 94795031 | 94795081 |
| chr9 | 94795545 | 94795595 |
| chr9 | 95209693 | 95209743 |
| chr9 | 101517914 | 101517964 |
| chr9 | 109741047 | 109741097 |
| chr9 | 109741216 | 109741266 |
| chr9 | 111228811 | 111228861 |
| chr12 | 1535895 | 1535945 |
| chr9 | 127166810 | 127166860 |
| chr9 | 127167564 | 127167614 |
| chr12 | 2984616 | 2984666 |
| chr12 | 3082145 | 3082195 |
| chr12 | 9792713 | 9792763 |
| chr12 | 24017923 | 24017973 |
| chr12 | 24018133 | 24018183 |
| chr12 | 27217688 | 27217738 |
| chr12 | 95652299 | 95652349 |
| chr12 | 100800118 | 100800168 |
| chr10 | 30559218 | 30559268 |
| chr12 | 113366830 | 113366880 |
| chr12 | 121478490 | 121478540 |
| chr10 | 34408114 | 34408164 |
| chr13 | 20987187 | 20987237 |
| chr13 | 36333253 | 36333303 |
| chr13 | 44565163 | 44565213 |
| chr13 | 56461789 | 56461839 |
| chr13 | 67136503 | 67136553 |
| chr13 | 81824455 | 81824505 |
| chr13 | 82238632 | 82238682 |
| chr13 | 104619850 | 104619900 |
| chr13 | 111528594 | 111528644 |
| chr13 | 111869692 | 111869742 |
| chr14 | 18670218 | 18670268 |
| chr14 | 20032249 | 20032299 |
| chr14 | 33434071 | 33434121 |
| chr14 | 35076050 | 35076100 |
| chr14 | 46229206 | 46229256 |
| chr14 | 46516128 | 46516178 |
| chr14 | 51155231 | 51155281 |
| chr14 | 77906467 | 77906517 |
| chr14 | 86011597 | 86011647 |
| chr14 | 97134833 | 97134883 |
| chr14 | 102520495 | 102520545 |
| chr14 | 104819282 | 104819332 |
| chr15 | 19266489 | 19266539 |
| chr15 | 19763976 | 19764026 |
| chr15 | 21592628 | 21592678 |
| chr15 | 21748934 | 21748984 |
| chr15 | 21894279 | 21894329 |
| chr15 | 22270328 | 22270378 |
| chr15 | 23626101 | 23626151 |
| chr15 | 30989900 | 30989950 |
| chr15 | 38698643 | 38698693 |
| chr15 | 50358067 | 50358117 |
| chr15 | 53169043 | 53169093 |
| chr10 | 68976094 | 68976144 |
| chr10 | 68976807 | 68976857 |
| chr15 | 88397088 | 88397138 |
| chr15 | 99127361 | 99127411 |
| chr16 | 26886043 | 26886093 |
| chr16 | 34892671 | 34892721 |
| chr16 | 53561964 | 53562014 |
| chr16 | 80652544 | 80652594 |
| chr16 | 87167095 | 87167145 |
| chr10 | 75252045 | 75252095 |
| chr17 | 2553752 | 2553802 |
| chr17 | 8772966 | 8773016 |
| chr17 | 10754150 | 10754200 |
| chr17 | 12105194 | 12105244 |
| chr17 | 14078969 | 14079019 |

Table 15AC. Repeat Category L1PA10

| | | |
|---|---|---|
| chr17 | 50252538 | 50252588 |
| chr17 | 64117804 | 64117854 |
| chr18 | 4079513 | 4079563 |
| chr10 | 94415335 | 94415385 |
| chr18 | 12456556 | 12456606 |
| chr18 | 29220905 | 29220955 |
| chr18 | 30726612 | 30726662 |
| chr18 | 31091641 | 31091691 |
| chr18 | 31091750 | 31091800 |
| chr18 | 53750610 | 53750660 |
| chr18 | 54250097 | 54250147 |
| chr18 | 61488063 | 61488113 |
| chr18 | 61487950 | 61488000 |
| chr18 | 66585508 | 66585558 |
| chr18 | 72470759 | 72470809 |
| chr18 | 73023138 | 73023188 |
| chr19 | 2455985 | 2456035 |
| chr19 | 5329739 | 5329789 |
| chr19 | 6535638 | 6535688 |
| chr19 | 15717376 | 15717426 |
| chr19 | 15826061 | 15826111 |
| chr19 | 16185589 | 16185639 |
| chr19 | 21017509 | 21017559 |
| chr19 | 21710382 | 21710432 |
| chr19 | 22387325 | 22387375 |
| chr19 | 22770247 | 22770297 |
| chr19 | 35681603 | 35681653 |
| chr19 | 46698982 | 46699032 |
| chr19 | 50057613 | 50057663 |
| chr19 | 50057768 | 50057818 |
| chr19 | 53241724 | 53241774 |
| chr19 | 56059362 | 56059412 |
| chr19 | 59498707 | 59498757 |
| chr19 | 59498636 | 59498686 |
| chr19 | 60223142 | 60223192 |
| chr19 | 61561616 | 61561666 |
| chr19 | 62662541 | 62662591 |
| chr19 | 62663313 | 62663363 |
| chr1 | 9823107 | 9823157 |
| chr10 | 121552460 | 121552510 |
| chr1 | 41845770 | 41845820 |
| chr1 | 56206883 | 56206933 |
| chr1 | 62253220 | 62253270 |
| chr1 | 69396699 | 69396749 |
| chr1 | 91159585 | 91159635 |
| chr1 | 152965514 | 152965564 |
| chr1 | 152965426 | 152965476 |
| chr1 | 159651111 | 159651161 |
| chr1 | 170471915 | 170471965 |
| chr1 | 186026359 | 186026409 |
| chr1 | 186070465 | 186070515 |
| chr1 | 186072147 | 186072197 |
| chr1 | 192037347 | 192037397 |
| chr1 | 211723853 | 211723903 |
| chr1 | 236049393 | 236049443 |
| chr1 | 238720922 | 238720972 |
| chr1 | 244274822 | 244274872 |
| chr20 | 5309735 | 5309785 |
| chr20 | 45008622 | 45008672 |
| chr20 | 53337013 | 53337063 |
| chr20 | 53576552 | 53576602 |
| chr20 | 60591086 | 60591136 |
| chr21 | 16815403 | 16815453 |
| chr21 | 17173591 | 17173641 |
| chr21 | 23368632 | 23368682 |
| chr21 | 26837781 | 26837831 |
| chr21 | 29684534 | 29684584 |
| chr11 | 435468 | 435518 |
| chr21 | 36192720 | 36192770 |
| chr21 | 45592947 | 45592997 |
| chr22 | 15715877 | 15715927 |
| chr22 | 22297851 | 22297901 |
| chr22 | 25011253 | 25011303 |
| chr22 | 25508924 | 25508974 |
| chr22 | 30865552 | 30865602 |
| chr22 | 32741046 | 32741096 |
| chr22 | 34908292 | 34908342 |
| chr22 | 43694077 | 43694127 |
| chrX | 2954666 | 2954716 |
| chrX | 3076500 | 3076550 |
| chrX | 4191602 | 4191652 |
| chrX | 7255205 | 7255255 |
| chrX | 20253720 | 20253770 |
| chrX | 33895785 | 33895835 |
| chrX | 34630488 | 34630538 |
| chrX | 39883260 | 39883310 |
| chrX | 44121086 | 44121136 |
| chrX | 49395823 | 49395873 |
| chrX | 54046144 | 54046194 |
| chrX | 55107485 | 55107535 |
| chrX | 62105939 | 62105989 |
| chrX | 63644037 | 63644087 |
| chrX | 71467591 | 71467641 |
| chrX | 76886030 | 76886080 |
| chrX | 81466896 | 81466946 |
| chrX | 85136796 | 85136846 |
| chrX | 86727887 | 86727937 |
| chrX | 91523785 | 91523835 |
| chrX | 100694030 | 100694080 |
| chrX | 100703245 | 100703295 |
| chrX | 106678951 | 106679001 |
| chrX | 106862532 | 106862582 |
| chrX | 106866712 | 106866762 |
| chrX | 111351040 | 111351090 |
| chrX | 121997400 | 121997450 |
| chrX | 126437759 | 126437809 |
| chr11 | 13568968 | 13569018 |
| chrX | 152357970 | 152358020 |
| chrY | 7125685 | 7125735 |
| chr11 | 15325856 | 15325906 |
| chr2 | 3277555 | 3277605 |
| chr2 | 12230618 | 12230668 |
| chr2 | 28528510 | 28528560 |
| chr11 | 18930183 | 18930233 |
| chr2 | 52173576 | 52173626 |
| chr2 | 55912514 | 55912564 |
| chr2 | 57351994 | 57352044 |
| chr10 | 11730751 | 11730801 |
| chr2 | 63756402 | 63756452 |
| chr2 | 70668251 | 70668301 |
| chr2 | 75925176 | 75925226 |
| chr2 | 83969639 | 83969689 |
| chr2 | 95950686 | 95950736 |
| chr2 | 95977329 | 95977379 |
| chr2 | 98599521 | 98599571 |
| chr2 | 98605334 | 98605384 |
| chr2 | 98605388 | 98605438 |
| chr2 | 103934530 | 103934580 |
| chr2 | 136303402 | 136303452 |
| chr2 | 137945960 | 137946010 |
| chr11 | 35433970 | 35434020 |
| chr2 | 168877826 | 168877876 |
| chr2 | 170895419 | 170895469 |
| chr2 | 173176591 | 173176641 |
| chr2 | 178420623 | 178420673 |
| chr2 | 179348838 | 179348888 |
| chr2 | 184773627 | 184773677 |
| chr2 | 194811863 | 194811913 |
| chr2 | 196645647 | 196645697 |
| chr2 | 224020361 | 224020411 |
| chr2 | 231409748 | 231409798 |
| chr2 | 238467594 | 238467644 |
| chr3 | 23061476 | 23061526 |
| chr3 | 52948323 | 52948373 |
| chr3 | 73701793 | 73701843 |
| chr3 | 74955989 | 74956039 |
| chr3 | 82081017 | 82081067 |
| chr3 | 84219795 | 84219845 |
| chr3 | 89845581 | 89845631 |
| chr3 | 95717230 | 95717280 |
| chr3 | 119639148 | 119639198 |
| chr3 | 121276915 | 121276965 |
| chr3 | 122152164 | 122152214 |
| chr3 | 127811245 | 127811295 |
| chr3 | 138991620 | 138991670 |
| chr3 | 154288327 | 154288377 |
| chr3 | 159154087 | 159154137 |
| chr3 | 159153965 | 159154015 |
| chr11 | 62632600 | 62632650 |
| chr4 | 8849074 | 8849124 |
| chr4 | 12544457 | 12544507 |
| chr4 | 17437746 | 17437796 |
| chr4 | 25037130 | 25037180 |
| chr4 | 28651559 | 28651609 |
| chr4 | 29386853 | 29386903 |
| chr4 | 37360323 | 37360373 |
| chr4 | 37358771 | 37358821 |
| chr4 | 42045510 | 42045560 |
| chr4 | 43086551 | 43086601 |
| chr4 | 48053538 | 48053588 |
| chr4 | 48053470 | 48053520 |
| chr4 | 56419008 | 56419058 |
| chr4 | 68842604 | 68842654 |
| chr4 | 81722842 | 81722892 |
| chr11 | 65596523 | 65596573 |
| chr4 | 108959144 | 108959194 |
| chr4 | 129101735 | 129101785 |
| chr4 | 129101547 | 129101597 |
| chr4 | 133724610 | 133724660 |
| chr4 | 135139711 | 135139761 |
| chr4 | 154403250 | 154403300 |
| chr4 | 157071766 | 157071816 |
| chr4 | 167633948 | 167633998 |
| chr4 | 176463337 | 176463387 |
| chr4 | 188888579 | 188888629 |
| chr4 | 189339316 | 189339366 |
| chr5 | 994927 | 994977 |
| chr5 | 3550564 | 3550614 |
| chr5 | 45065653 | 45065703 |
| chr5 | 63603420 | 63603470 |
| chr5 | 71655759 | 71655809 |
| chr5 | 75231846 | 75231896 |
| chr5 | 94129429 | 94129479 |
| chr5 | 96784007 | 96784057 |
| chr5 | 102025234 | 102025284 |
| chr5 | 102024214 | 102024264 |
| chr5 | 117838679 | 117838729 |
| chr5 | 130308709 | 130308759 |
| chr5 | 142939442 | 142939492 |
| chr5 | 151789792 | 151789842 |
| chr5 | 154733518 | 154733568 |
| chr5 | 160231293 | 160231343 |
| chr5 | 166161133 | 166161183 |
| chr5 | 168779892 | 168779942 |
| chr5 | 171729269 | 171729319 |
| chr6 | 18615023 | 18615073 |
| chr6 | 49396411 | 49396461 |
| chr6 | 49817521 | 49817571 |
| chr6 | 54921254 | 54921304 |
| chr11 | 88532330 | 88532380 |
| chr6 | 64291721 | 64291771 |
| chr11 | 90401194 | 90401244 |
| chr6 | 74747115 | 74747165 |
| chr6 | 84191454 | 84191504 |
| chr11 | 92557848 | 92557898 |
| chr6 | 108390913 | 108390963 |
| chr6 | 120158951 | 120159001 |
| chr11 | 96607504 | 96607554 |
| chr6 | 137335810 | 137335860 |
| chr6 | 137867341 | 137867391 |
| chr6 | 154908871 | 154908921 |
| chr6 | 156878108 | 156878158 |
| chr11 | 104895418 | 104895468 |
| chr11 | 105369288 | 105369338 |
| chr7 | 4174729 | 4174779 |
| chr7 | 9474833 | 9474883 |
| chr7 | 23845278 | 23845328 |
| chr7 | 38727087 | 38727137 |
| chr7 | 49629483 | 49629533 |
| chr7 | 70627633 | 70627683 |
| chr7 | 76576139 | 76576189 |
| chr7 | 79570788 | 79570838 |
| chr7 | 81341103 | 81341153 |
| chr7 | 90940717 | 90940767 |
| chr7 | 96299412 | 96299462 |
| chr7 | 109534682 | 109534732 |
| chr7 | 111747858 | 111747908 |
| chr7 | 118415833 | 118415883 |
| chr7 | 130099509 | 130099559 |
| chr7 | 130694642 | 130694692 |
| chr7 | 133276883 | 133276933 |
| chr7 | 143129491 | 143129541 |
| chr7 | 156755506 | 156755556 |
| chr8 | 5759543 | 5759593 |
| chr8 | 9061830 | 9061880 |
| chr8 | 12021480 | 12021530 |
| chr8 | 13993941 | 13993991 |
| chr8 | 19075861 | 19075911 |
| chr8 | 27939134 | 27939184 |
| chr8 | 31205423 | 31205473 |
| chr8 | 42638494 | 42638544 |
| chr8 | 88042896 | 88042946 |
| chr8 | 104602371 | 104602421 |
| chr8 | 110299555 | 110299605 |
| chr8 | 111530610 | 111530660 |
| chr8 | 118745802 | 118745852 |
| chr8 | 118976138 | 118976188 |
| chr11 | 129068945 | 129068995 |
| chr11 | 129067730 | 129067780 |
| chr8 | 126880020 | 126880070 |
| chr8 | 136842059 | 136842109 |
| chr8 | 141588892 | 141588942 |
| chr8 | 144293928 | 144293978 |
| chr9 | 10818414 | 10818464 |
| chr9 | 22219347 | 22219397 |
| chr11 | 132916409 | 132916459 |
| chr9 | 28107906 | 28107956 |
| chr9 | 75776286 | 75776336 |
| chr9 | 80136978 | 80137028 |
| chr9 | 82198898 | 82198948 |
| chr9 | 85810549 | 85810599 |
| chr9 | 91521400 | 91521450 |
| chr9 | 92634124 | 92634174 |
| chr9 | 92634716 | 92634766 |
| chr9 | 92634904 | 92634954 |
| chr9 | 100746438 | 100746488 |
| chr9 | 106249708 | 106249758 |
| chr9 | 111134285 | 111134335 |
| chr9 | 118502866 | 118502916 |
| chr10 | 22192963 | 22193013 |
| chr10 | 22203324 | 22203374 |
| chr12 | 8571561 | 8571611 |
| chr10 | 23064880 | 23064930 |
| chr12 | 18005478 | 18005528 |
| chr12 | 58482225 | 58482275 |
| chr12 | 62843530 | 62843580 |
| chr12 | 63232943 | 63232993 |
| chr12 | 63674690 | 63674740 |
| chr12 | 79469345 | 79469395 |
| chr12 | 92455143 | 92455193 |
| chr12 | 92587902 | 92587952 |
| chr12 | 97324023 | 97324073 |
| chr12 | 114737322 | 114737372 |
| chr12 | 116532463 | 116532513 |
| chr10 | 33077282 | 33077332 |
| chr13 | 36377542 | 36377592 |
| chr13 | 37492442 | 37492492 |
| chr13 | 54818244 | 54818294 |
| chr13 | 56500371 | 56500421 |
| chr13 | 86816615 | 86816665 |
| chr13 | 88428448 | 88428498 |
| chr13 | 89991753 | 89991803 |
| chr13 | 101169940 | 101169990 |
| chr13 | 101842647 | 101842697 |
| chr14 | 36907001 | 36907051 |
| chr14 | 42116706 | 42116756 |
| chr14 | 57386257 | 57386307 |
| chr14 | 65513765 | 65513815 |
| chr10 | 53954142 | 53954192 |
| chr14 | 85344044 | 85344094 |
| chr14 | 90014834 | 90014884 |
| chr10 | 61422310 | 61422360 |
| chr15 | 25486223 | 25486273 |
| chr15 | 27036149 | 27036199 |
| chr15 | 28743454 | 28743504 |
| chr15 | 31586315 | 31586365 |
| chr15 | 31585352 | 31585402 |
| chr15 | 35167266 | 35167316 |
| chr15 | 37013914 | 37013964 |
| chr15 | 57521773 | 57521823 |
| chr15 | 61456669 | 61456719 |
| chr15 | 62278458 | 62278508 |
| chr15 | 77627701 | 77627751 |
| chr15 | 94715304 | 94715354 |
| chr15 | 94715171 | 94715221 |
| chr16 | 6135854 | 6135904 |
| chr16 | 7224649 | 7224699 |
| chr16 | 14228681 | 14228731 |
| chr16 | 27873816 | 27873866 |
| chr16 | 28975028 | 28975078 |
| chr16 | 30206560 | 30206610 |
| chr16 | 47519498 | 47519548 |
| chr16 | 61932828 | 61932878 |
| chr16 | 62826156 | 62826206 |
| chr16 | 67920095 | 67920145 |
| chr16 | 67922983 | 67923033 |
| chr16 | 81218942 | 81218992 |
| chr16 | 82404524 | 82404574 |
| chr16 | 84287665 | 84287715 |
| chr16 | 84369434 | 84369484 |
| chr16 | 85869268 | 85869318 |
| chr17 | 29416809 | 29416859 |

Table 15AD. Repeat Category L1PA12

| | | |
|---|---|---|
| chr18 | 46199029 | 46199079 |
| chr19 | 23272870 | 23272920 |
| chr19 | 44807064 | 44807114 |
| chr10 | 108291957 | 108292007 |
| chr10 | 110674802 | 110674852 |
| chr19 | 57328505 | 57328555 |
| chr10 | 117120268 | 117120318 |
| chr1 | 20641116 | 20641166 |
| chr1 | 49315162 | 49315212 |
| chr1 | 78301519 | 78301569 |
| chr1 | 78301148 | 78301198 |
| chr1 | 102545920 | 102545970 |
| chr1 | 158411184 | 158411234 |
| chr1 | 158411565 | 158411615 |
| chr1 | 166703864 | 166703914 |
| chr1 | 166703135 | 166703185 |
| chr1 | 171017150 | 171017200 |
| chr1 | 172067233 | 172067283 |
| chr1 | 187084486 | 187084536 |
| chr1 | 187088643 | 187088693 |
| chr1 | 195050782 | 195050832 |
| chr1 | 204666890 | 204666940 |
| chr1 | 217483013 | 217483063 |
| chr1 | 222231440 | 222231490 |
| chr1 | 222236615 | 222236665 |
| chr20 | 23924943 | 23924993 |
| chr10 | 134432443 | 134432493 |
| chr21 | 19170283 | 19170333 |
| chr21 | 23855081 | 23855131 |
| chr21 | 34760579 | 34760629 |
| chr22 | 27369731 | 27369781 |
| chr11 | 2225128 | 2225178 |
| chrX | 27405708 | 27405758 |
| chrX | 33794689 | 33794739 |
| chrX | 52807949 | 52807999 |
| chrX | 55907603 | 55907653 |
| chrX | 74094454 | 74094504 |
| chrX | 77543283 | 77543333 |
| chrX | 81846023 | 81846073 |
| chrX | 86814406 | 86814456 |
| chrX | 102102950 | 102103000 |
| chrX | 103391156 | 103391206 |
| chrX | 110400203 | 110400253 |
| chrX | 145061491 | 145061541 |
| chrY | 20468938 | 20468988 |
| chr2 | 49032613 | 49032663 |
| chr11 | 22007891 | 22007941 |
| chr2 | 53974216 | 53974266 |
| chr2 | 72514685 | 72514735 |
| chr2 | 95393385 | 95393435 |
| chr2 | 95978371 | 95978421 |
| chr2 | 95977329 | 95977379 |
| chr2 | 97729789 | 97729839 |
| chr2 | 117881546 | 117881596 |
| chr2 | 157773445 | 157773495 |
| chr3 | 957334 | 957384 |
| chr3 | 3656825 | 3656875 |
| chr3 | 33542740 | 33542790 |
| chr11 | 48415638 | 48415688 |
| chr11 | 49145062 | 49145112 |
| chr3 | 55232032 | 55232082 |
| chr3 | 99968580 | 99968630 |
| chr3 | 105143736 | 105143786 |
| chr3 | 106335848 | 106335898 |
| chr3 | 117584672 | 117584722 |
| chr3 | 120545567 | 120545617 |
| chr3 | 141536531 | 141536581 |
| chr3 | 142233691 | 142233741 |
| chr3 | 148189761 | 148189811 |
| chr11 | 62897523 | 62897573 |
| chr3 | 190062049 | 190062099 |
| chr4 | 70275199 | 70275249 |
| chr4 | 94632494 | 94632544 |
| chr4 | 118209521 | 118209571 |
| chr4 | 133724610 | 133724660 |
| chr4 | 145082770 | 145082820 |
| chr4 | 172920518 | 172920568 |
| chr4 | 190618920 | 190618970 |
| chr5 | 17706614 | 17706664 |
| chr5 | 23756624 | 23756674 |
| chr5 | 54998119 | 54998169 |
| chr5 | 55696299 | 55696349 |
| chr5 | 60407963 | 60408013 |
| chr5 | 97063991 | 97064041 |
| chr5 | 126571703 | 126571753 |
| chr5 | 146250823 | 146250873 |
| chr5 | 156816031 | 156816081 |
| chr11 | 86321267 | 86321317 |
| chr11 | 86321053 | 86321103 |
| chr6 | 64187918 | 64187968 |
| chr6 | 71054158 | 71054208 |
| chr6 | 77236872 | 77236922 |
| chr6 | 79334988 | 79335038 |
| chr6 | 94213910 | 94213960 |
| chr6 | 95633808 | 95633858 |
| chr6 | 101776325 | 101776375 |
| chr6 | 145798799 | 145798849 |
| chr6 | 161231654 | 161231704 |
| chr11 | 110251821 | 110251871 |
| chr7 | 62000829 | 62000879 |
| chr7 | 96944463 | 96944513 |
| chr7 | 112200554 | 112200604 |
| chr7 | 150703910 | 150703960 |
| chr7 | 150703637 | 150703687 |
| chr7 | 151735665 | 151735715 |
| chr8 | 39576240 | 39576290 |
| chr8 | 47977137 | 47977187 |
| chr8 | 71663523 | 71663573 |
| chr8 | 89453741 | 89453791 |
| chr8 | 91171978 | 91172028 |
| chr8 | 116108333 | 116108383 |
| chr8 | 118182122 | 118182172 |
| chr9 | 12737263 | 12737313 |
| chr9 | 15345955 | 15346005 |
| chr9 | 30290641 | 30290691 |
| chr10 | 20554847 | 20554897 |
| chr12 | 7743841 | 7743891 |
| chr12 | 19827970 | 19828020 |
| chr12 | 85238799 | 85238849 |
| chr13 | 50840233 | 50840283 |
| chr13 | 69406663 | 69406713 |
| chr13 | 72258592 | 72258642 |
| chr13 | 85197118 | 85197168 |
| chr13 | 87778528 | 87778578 |
| chr14 | 19626673 | 19626723 |
| chr14 | 26723679 | 26723729 |
| chr14 | 65125805 | 65125855 |
| chr10 | 51598494 | 51598544 |
| chr15 | 29122117 | 29122167 |
| chr15 | 32055613 | 32055663 |
| chr15 | 37799915 | 37799965 |
| chr15 | 37818035 | 37818085 |
| chr15 | 44271632 | 44271682 |
| chr15 | 52650190 | 52650240 |
| chr10 | 74028636 | 74028686 |
| chr16 | 84617007 | 84617057 |

Table 15AE. Repeat Category L1PA15-16

| | | |
|---|---|---|
| chr18 | 11422702 | 11422752 |
| chr19 | 41682823 | 41682873 |
| chr1 | 74144671 | 74144721 |
| chr1 | 105276452 | 105276502 |
| chr1 | 155715641 | 155715691 |
| chr20 | 33447122 | 33447172 |
| chr21 | 40506888 | 40506938 |
| chr22 | 33354409 | 33354459 |
| chrX | 33785012 | 33785062 |
| chrX | 56422241 | 56422291 |
| chrX | 61524868 | 61524918 |
| chrX | 74774697 | 74774747 |
| chrX | 79424014 | 79424064 |
| chrX | 81874559 | 81874609 |
| chrX | 86609318 | 86609368 |
| chrX | 87993717 | 87993767 |
| chrX | 141318714 | 141318764 |
| chrY | 3193220 | 3193270 |
| chr2 | 128115572 | 128115622 |
| chr2 | 128117464 | 128117514 |
| chr2 | 132947303 | 132947353 |
| chr2 | 165387330 | 165387380 |
| chr2 | 219192377 | 219192427 |
| chr2 | 219192916 | 219192966 |
| chr2 | 219192978 | 219193028 |
| chr11 | 49092742 | 49092792 |
| chr3 | 138160521 | 138160571 |
| chr3 | 197913520 | 197913570 |
| chr4 | 11867802 | 11867852 |
| chr4 | 33354901 | 33354951 |
| chr10 | 794252 | 794302 |
| chr4 | 67341126 | 67341176 |
| chr10 | 800533 | 800583 |
| chr4 | 81036713 | 81036763 |
| chr4 | 122861549 | 122861599 |
| chr4 | 128968843 | 128968893 |
| chr4 | 152376078 | 152376128 |
| chr5 | 33148491 | 33148541 |
| chr5 | 86399411 | 86399461 |
| chr5 | 144151334 | 144151384 |
| chr11 | 81932315 | 81932365 |
| chr11 | 89149415 | 89149465 |
| chr6 | 69062422 | 69062472 |
| chr6 | 77831860 | 77831910 |
| chr6 | 83989354 | 83989404 |
| chr6 | 93154335 | 93154385 |
| chr6 | 93724630 | 93724680 |
| chr6 | 109959505 | 109959555 |
| chr6 | 120244873 | 120244923 |
| chr7 | 55638059 | 55638109 |
| chr7 | 80369034 | 80369084 |
| chr7 | 81799084 | 81799134 |
| chr8 | 89654440 | 89654490 |
| chr12 | 45665176 | 45665226 |
| chr12 | 87611105 | 87611155 |
| chr13 | 23344692 | 23344742 |
| chr14 | 24621008 | 24621058 |
| chr14 | 40571928 | 40571978 |
| chr14 | 90217577 | 90217627 |
| chr14 | 90218239 | 90218289 |
| chr14 | 105144891 | 105144941 |
| chr15 | 31227724 | 31227774 |
| chr15 | 33089198 | 33089248 |
| chr15 | 84608074 | 84608124 |

Table 15AF. Repeat Category L1PB1

| | | |
|---|---|---|
| chr17 | 43755737 | 43755787 |
| chr17 | 46001374 | 46001424 |
| chr17 | 50660721 | 50660771 |
| chr17 | 57191266 | 57191316 |
| chr17 | 59661102 | 59661152 |
| chr17 | 64166439 | 64166489 |
| chr17 | 69541296 | 69541346 |
| chr10 | 91212534 | 91212584 |
| chr18 | 2236460 | 2236510 |
| chr18 | 2847981 | 2848031 |
| chr18 | 4682178 | 4682228 |
| chr18 | 7324183 | 7324233 |
| chr18 | 7336584 | 7336634 |
| chr18 | 8895530 | 8895580 |
| chr18 | 10983990 | 10984040 |
| chr18 | 11598689 | 11598739 |
| chr18 | 12910973 | 12911023 |
| chr18 | 13155807 | 13155857 |
| chr18 | 13161119 | 13161169 |
| chr18 | 13566053 | 13566103 |
| chr18 | 13566108 | 13566158 |
| chr18 | 17548485 | 17548535 |
| chr18 | 19861327 | 19861377 |
| chr18 | 23700432 | 23700482 |
| chr18 | 26298451 | 26298501 |
| chr18 | 44181934 | 44181984 |
| chr18 | 51975422 | 51975472 |
| chr18 | 52699455 | 52699505 |
| chr18 | 52913805 | 52913855 |
| chr18 | 57081932 | 57081982 |
| chr10 | 98202047 | 98202097 |
| chr18 | 67578731 | 67578781 |
| chr18 | 72799786 | 72799836 |
| chr19 | 2075531 | 2075581 |
| chr19 | 7187721 | 7187771 |
| chr19 | 11962301 | 11962351 |
| chr19 | 13985281 | 13985331 |
| chr19 | 14845753 | 14845803 |
| chr19 | 15826789 | 15826839 |
| chr19 | 15828452 | 15828502 |
| chr19 | 20573288 | 20573338 |
| chr19 | 21325372 | 21325422 |
| chr19 | 23331413 | 23331463 |
| chr19 | 23674870 | 23674920 |
| chr19 | 34831840 | 34831890 |
| chr19 | 49316765 | 49316815 |
| chr19 | 49758824 | 49758874 |
| chr19 | 51508649 | 51508699 |
| chr10 | 113238050 | 113238100 |
| chr19 | 61834161 | 61834211 |
| chr19 | 61833746 | 61833796 |
| chr1 | 5445208 | 5445258 |
| chr1 | 15666552 | 15666602 |
| chr1 | 17731011 | 17731061 |
| chr10 | 120719350 | 120719400 |
| chr1 | 31427906 | 31427956 |
| chr1 | 38827855 | 38827905 |
| chr1 | 38898322 | 38898372 |
| chr1 | 42772858 | 42772908 |
| chr1 | 49186187 | 49186237 |
| chr1 | 51614761 | 51614811 |
| chr1 | 51614642 | 51614692 |
| chr1 | 53649268 | 53649318 |
| chr1 | 57405900 | 57405950 |
| chr1 | 58324350 | 58324400 |
| chr10 | 123533483 | 123533533 |
| chr1 | 59149373 | 59149423 |
| chr1 | 62303635 | 62303685 |
| chr1 | 62579861 | 62579911 |
| chr1 | 78659185 | 78659235 |
| chr1 | 79566669 | 79566719 |
| chr1 | 81571985 | 81572035 |
| chr1 | 83188625 | 83188675 |
| chr1 | 86233589 | 86233639 |
| chr1 | 86435272 | 86435322 |
| chr10 | 6707061 | 6707111 |
| chr1 | 92038421 | 92038471 |
| chr1 | 93871275 | 93871325 |
| chr1 | 114046013 | 114046063 |
| chr1 | 115856007 | 115856057 |
| chr1 | 115936482 | 115936532 |
| chr1 | 116435726 | 116435776 |
| chr1 | 116677552 | 116677602 |
| chr1 | 144070160 | 144070210 |
| chr1 | 144807769 | 144807819 |
| chr10 | 128018174 | 128018224 |
| chr1 | 158032935 | 158032985 |
| chr1 | 160000209 | 160000259 |
| chr1 | 160025795 | 160025845 |
| chr1 | 160025945 | 160025995 |
| chr1 | 160026187 | 160026237 |
| chr1 | 160026297 | 160026347 |
| chr1 | 160026485 | 160026535 |
| chr1 | 163253446 | 163253496 |
| chr1 | 170949271 | 170949321 |
| chr1 | 176884082 | 176884132 |
| chr1 | 195688142 | 195688192 |
| chr1 | 196262538 | 196262588 |
| chr1 | 211342635 | 211342685 |
| chr1 | 225493194 | 225493244 |
| chr1 | 231408349 | 231408399 |
| chr1 | 233170893 | 233170943 |
| chr1 | 233407225 | 233407275 |
| chr1 | 233594634 | 233594684 |
| chr1 | 234488099 | 234488149 |
| chr1 | 235249412 | 235249462 |
| chr20 | 12748493 | 12748543 |
| chr20 | 15676080 | 15676130 |
| chr20 | 15676002 | 15676052 |
| chr20 | 18206219 | 18206269 |
| chr20 | 20103164 | 20103214 |
| chr20 | 20101860 | 20101910 |
| chr10 | 133332059 | 133332109 |
| chr20 | 22160382 | 22160432 |
| chr20 | 24666744 | 24666794 |
| chr20 | 52703480 | 52703530 |
| chr20 | 58215431 | 58215481 |
| chr20 | 59746536 | 59746586 |
| chr21 | 15891494 | 15891544 |
| chr21 | 18189300 | 18189350 |
| chr21 | 28219920 | 28219970 |
| chr21 | 29133737 | 29133787 |
| chr21 | 29912214 | 29912264 |
| chr21 | 32152091 | 32152141 |
| chr21 | 36124695 | 36124745 |
| chr21 | 43956269 | 43956319 |
| chr21 | 46865594 | 46865644 |
| chr21 | 46864097 | 46864147 |
| chr22 | 14677823 | 14677873 |
| chr22 | 14702360 | 14702410 |
| chr11 | 1347341 | 1347391 |
| chr22 | 21114065 | 21114115 |
| chr22 | 23521405 | 23521455 |
| chr22 | 25218413 | 25218463 |
| chr22 | 27275106 | 27275156 |
| chr22 | 30488722 | 30488772 |
| chr22 | 32172514 | 32172564 |
| chr22 | 33276234 | 33276284 |
| chr22 | 34728810 | 34728860 |
| chrX | 2422990 | 2423040 |
| chrX | 8163949 | 8163999 |
| chrX | 8396565 | 8396615 |
| chrX | 14514370 | 14514420 |
| chrX | 18601684 | 18601734 |
| chrX | 18601290 | 18601340 |
| chrX | 20198245 | 20198295 |
| chrX | 27384656 | 27384706 |
| chrX | 44660816 | 44660866 |
| chrX | 52665125 | 52665175 |
| chrX | 53627305 | 53627355 |
| chrX | 54036977 | 54037027 |
| chrX | 55717598 | 55717648 |
| chrX | 61391121 | 61391171 |
| chrX | 64833815 | 64833865 |
| chrX | 67357280 | 67357330 |
| chrX | 72723253 | 72723303 |
| chrX | 72729012 | 72729062 |
| chrX | 73254840 | 73254890 |
| chrX | 73318379 | 73318429 |
| chrX | 73715385 | 73715435 |
| chrX | 74306383 | 74306433 |
| chrX | 76200194 | 76200244 |
| chrX | 76789312 | 76789362 |
| chrX | 89135662 | 89135712 |
| chrX | 89903043 | 89903093 |
| chrX | 90186963 | 90187013 |
| chrX | 98349864 | 98349914 |
| chrX | 98630087 | 98630137 |
| chrX | 98783587 | 98783637 |
| chrX | 99353498 | 99353548 |
| chrX | 100938954 | 100939004 |
| chrX | 106012830 | 106012880 |
| chrX | 106855149 | 106855199 |
| chrX | 107052624 | 107052674 |
| chrX | 110596825 | 110596875 |
| chrX | 113717947 | 113717997 |
| chrX | 113719331 | 113719381 |
| chrX | 114023797 | 114023847 |
| chrX | 117979860 | 117979910 |
| chrX | 118731220 | 118731270 |
| chrX | 118894812 | 118894862 |
| chrX | 124762385 | 124762435 |
| chrX | 125655802 | 125655852 |
| chrX | 127811312 | 127811362 |
| chrX | 129769069 | 129769119 |
| chrX | 135619490 | 135619540 |
| chrX | 135624673 | 135624723 |
| chrX | 136108680 | 136108730 |
| chrX | 136252962 | 136253012 |
| chrX | 136841805 | 136841855 |
| chrX | 140505968 | 140506018 |
| chrX | 151811316 | 151811366 |
| chr11 | 15075443 | 15075493 |
| chr2 | 1416488 | 1416538 |
| chr2 | 6262206 | 6262256 |
| chr2 | 26166515 | 26166565 |
| chr2 | 29227178 | 29227228 |
| chr2 | 41762031 | 41762081 |
| chr2 | 53869561 | 53869611 |
| chr2 | 55279506 | 55279556 |
| chr2 | 63340913 | 63340963 |
| chr2 | 63483809 | 63483859 |
| chr2 | 65601667 | 65601717 |
| chr2 | 66230722 | 66230772 |
| chr11 | 24731470 | 24731520 |
| chr11 | 24730100 | 24730150 |
| chr2 | 68650600 | 68650650 |
| chr2 | 69099004 | 69099054 |
| chr2 | 70607662 | 70607712 |
| chr2 | 70610560 | 70610610 |
| chr11 | 25678668 | 25678718 |
| chr2 | 75733118 | 75733168 |
| chr2 | 79095235 | 79095285 |
| chr2 | 88358557 | 88358607 |
| chr2 | 95381111 | 95381161 |
| chr2 | 95518596 | 95518646 |
| chr2 | 99251489 | 99251539 |
| chr2 | 99255311 | 99255361 |
| chr2 | 112539179 | 112539229 |
| chr2 | 113949003 | 113949053 |
| chr2 | 128898223 | 128898273 |
| chr2 | 128904333 | 128904383 |
| chr2 | 132521352 | 132521402 |
| chr2 | 132543365 | 132543415 |
| chr2 | 135401904 | 135401954 |
| chr2 | 136258392 | 136258442 |
| chr2 | 136266830 | 136266880 |
| chr2 | 140382066 | 140382116 |
| chr2 | 149058988 | 149059038 |
| chr2 | 156008278 | 156008328 |
| chr2 | 158178082 | 158178132 |
| chr2 | 159026405 | 159026455 |
| chr2 | 159148908 | 159148958 |
| chr2 | 159203474 | 159203524 |
| chr2 | 159207973 | 159208023 |
| chr2 | 160989569 | 160989619 |
| chr2 | 168908072 | 168908122 |
| chr11 | 36156001 | 36156051 |
| chr2 | 173219620 | 173219670 |
| chr11 | 39059466 | 39059516 |
| chr2 | 196762315 | 196762365 |
| chr2 | 205577184 | 205577234 |
| chr2 | 207099436 | 207099486 |
| chr2 | 207651371 | 207651421 |
| chr2 | 209680011 | 209680061 |
| chr2 | 215785885 | 215785935 |
| chr2 | 216231881 | 216231931 |
| chr2 | 222644470 | 222644520 |
| chr2 | 228302818 | 228302868 |
| chr2 | 234712622 | 234712672 |
| chr2 | 236267806 | 236267856 |
| chr3 | 2833604 | 2833654 |
| chr3 | 19339631 | 19339681 |
| chr3 | 19871507 | 19871557 |
| chr3 | 20937962 | 20938012 |
| chr3 | 27169798 | 27169848 |
| chr3 | 28417759 | 28417809 |
| chr11 | 48409596 | 48409646 |
| chr3 | 44700339 | 44700389 |
| chr3 | 45376323 | 45376373 |
| chr3 | 50785237 | 50785287 |
| chr11 | 55695474 | 55695524 |
| chr11 | 55695159 | 55695209 |
| chr3 | 59008740 | 59008790 |
| chr3 | 72850990 | 72851040 |
| chr3 | 75034946 | 75034996 |
| chr3 | 75037648 | 75037698 |
| chr3 | 99091887 | 99091937 |
| chr3 | 99515231 | 99515281 |
| chr3 | 115310082 | 115310132 |
| chr3 | 119611698 | 119611748 |
| chr3 | 120084847 | 120084897 |
| chr3 | 123489659 | 123489709 |
| chr3 | 126843500 | 126843550 |
| chr3 | 132675941 | 132675991 |
| chr3 | 139592863 | 139592913 |
| chr3 | 143759607 | 143759657 |
| chr3 | 148672969 | 148673019 |
| chr3 | 152324205 | 152324255 |
| chr3 | 156760801 | 156760851 |
| chr3 | 159535650 | 159535700 |
| chr3 | 159538919 | 159538969 |
| chr3 | 177865952 | 177866002 |
| chr3 | 177929670 | 177929720 |
| chr3 | 191276997 | 191277047 |
| chr3 | 197228609 | 197228659 |
| chr4 | 9324336 | 9324386 |
| chr4 | 14084717 | 14084767 |
| chr4 | 14232860 | 14232910 |
| chr4 | 22227934 | 22227984 |
| chr4 | 39258041 | 39258091 |
| chr4 | 42691070 | 42691120 |
| chr4 | 45345615 | 45345665 |
| chr4 | 54873676 | 54873726 |
| chr4 | 67692210 | 67692260 |
| chr4 | 71326740 | 71326790 |
| chr4 | 81156810 | 81156860 |
| chr4 | 82793544 | 82793594 |
| chr4 | 87897280 | 87897330 |
| chr4 | 102763344 | 102763394 |
| chr4 | 103865331 | 103865381 |
| chr4 | 104117335 | 104117385 |
| chr4 | 106189552 | 106189602 |
| chr4 | 115521050 | 115521100 |
| chr4 | 116579222 | 116579272 |
| chr4 | 122358442 | 122358492 |
| chr4 | 124107099 | 124107149 |
| chr4 | 124166475 | 124166525 |
| chr4 | 129239899 | 129239949 |
| chr4 | 129505411 | 129505461 |
| chr4 | 133778329 | 133778379 |
| chr4 | 145181923 | 145181973 |
| chr4 | 147178271 | 147178321 |
| chr4 | 153229381 | 153229431 |
| chr4 | 154340329 | 154340379 |
| chr4 | 163788117 | 163788167 |
| chr4 | 163897128 | 163897178 |
| chr4 | 172596714 | 172596764 |
| chr4 | 175389140 | 175389190 |
| chr4 | 187999230 | 187999280 |
| chr4 | 189288590 | 189288640 |
| chr4 | 189547165 | 189547215 |
| chr4 | 189936423 | 189936473 |
| chr4 | 189936703 | 189936753 |
| chr4 | 189939739 | 189939789 |
| chr4 | 190390211 | 190390261 |
| chr4 | 190393381 | 190393431 |
| chr4 | 190739029 | 190739079 |
| chr5 | 9855769 | 9855819 |
| chr5 | 17050133 | 17050183 |
| chr5 | 17706614 | 17706664 |
| chr5 | 22580273 | 22580323 |
| chr5 | 25413578 | 25413628 |
| chr5 | 29693226 | 29693276 |
| chr10 | 14013027 | 14013077 |
| chr5 | 35792089 | 35792139 |
| chr5 | 35791614 | 35791664 |
| chr5 | 38052198 | 38052248 |
| chr5 | 54880452 | 54880502 |
| chr5 | 57464141 | 57464191 |
| chr5 | 59968543 | 59968593 |
| chr5 | 67067716 | 67067766 |
| chr5 | 76925310 | 76925360 |
| chr5 | 81340110 | 81340160 |
| chr5 | 81396870 | 81396920 |
| chr5 | 93178193 | 93178243 |
| chr5 | 98232040 | 98232090 |
| chr5 | 98295344 | 98295394 |
| chr5 | 103475119 | 103475169 |
| chr5 | 108647406 | 108647456 |
| chr5 | 111926933 | 111926983 |
| chr5 | 114562310 | 114562360 |
| chr11 | 71992986 | 71993036 |
| chr5 | 124174921 | 124174971 |
| chr5 | 133033910 | 133033960 |
| chr5 | 135857659 | 135857709 |
| chr5 | 135863809 | 135863859 |
| chr5 | 143224210 | 143224260 |
| chr5 | 145023685 | 145023735 |
| chr5 | 145297332 | 145297382 |
| chr11 | 73555479 | 73555529 |
| chr5 | 161760151 | 161760201 |
| chr5 | 169869670 | 169869720 |
| chr5 | 174081126 | 174081176 |
| chr5 | 175913778 | 175913828 |
| chr6 | 4543460 | 4543510 |
| chr6 | 4783093 | 4783143 |
| chr11 | 78568832 | 78568882 |
| chr6 | 28032241 | 28032291 |
| chr6 | 28555101 | 28555151 |
| chr6 | 29036152 | 29036202 |
| chr6 | 29039151 | 29039201 |
| chr6 | 29145861 | 29145911 |
| chr6 | 30545563 | 30545613 |
| chr6 | 30558998 | 30559048 |
| chr6 | 32309993 | 32310043 |
| chr6 | 33111365 | 33111415 |
| chr6 | 42509159 | 42509209 |
| chr6 | 44970396 | 44970446 |
| chr6 | 45311537 | 45311587 |
| chr6 | 45707305 | 45707355 |
| chr6 | 46795022 | 46795072 |
| chr6 | 47637383 | 47637433 |
| chr6 | 56804762 | 56804812 |
| chr6 | 64534791 | 64534841 |
| chr6 | 65413514 | 65413564 |
| chr11 | 90906773 | 90906823 |
| chr6 | 85040591 | 85040641 |
| chr6 | 87376520 | 87376570 |
| chr6 | 91584694 | 91584744 |
| chr6 | 102237346 | 102237396 |
| chr11 | 94421798 | 94421848 |
| chr6 | 111602739 | 111602789 |
| chr6 | 117381576 | 117381626 |
| chr6 | 117739390 | 117739440 |
| chr6 | 119793243 | 119793293 |
| chr6 | 119792990 | 119793040 |
| chr6 | 126670584 | 126670634 |
| chr11 | 96997256 | 96997306 |
| chr6 | 134509904 | 134509954 |
| chr6 | 135666863 | 135666913 |
| chr6 | 138358435 | 138358485 |
| chr11 | 97960274 | 97960324 |
| chr6 | 143947052 | 143947102 |
| chr6 | 148304728 | 148304778 |
| chr11 | 100061137 | 100061187 |
| chr6 | 152001290 | 152001340 |
| chr6 | 155689744 | 155689794 |
| chr6 | 160451934 | 160451984 |
| chr6 | 167394090 | 167394140 |
| chr6 | 169529605 | 169529655 |
| chr6 | 169529697 | 169529747 |
| chr11 | 107168599 | 107168649 |
| chr11 | 107182177 | 107182227 |
| chr11 | 107538184 | 107538234 |
| chr7 | 4169290 | 4169340 |
| chr7 | 4896093 | 4896143 |
| chr11 | 108986376 | 108986426 |
| chr7 | 6811043 | 6811093 |
| chr7 | 11293871 | 11293921 |
| chr7 | 17819588 | 17819638 |
| chr7 | 25811936 | 25811986 |
| chr7 | 35464247 | 35464297 |
| chr7 | 36921174 | 36921224 |
| chr7 | 37060033 | 37060083 |
| chr7 | 41081366 | 41081416 |
| chr7 | 42215216 | 42215266 |
| chr11 | 112894632 | 112894682 |
| chr7 | 46889747 | 46889797 |
| chr7 | 47701973 | 47702023 |
| chr7 | 55917478 | 55917528 |
| chr7 | 63661619 | 63661669 |
| chr7 | 63717138 | 63717188 |
| chr7 | 67250919 | 67250969 |
| chr7 | 76005825 | 76005875 |
| chr7 | 80615869 | 80615919 |
| chr7 | 86984141 | 86984191 |
| chr7 | 89676151 | 89676201 |
| chr7 | 90775449 | 90775499 |
| chr7 | 94109542 | 94109592 |
| chr7 | 94418375 | 94418425 |
| chr7 | 94417781 | 94417831 |
| chr7 | 96315498 | 96315548 |
| chr7 | 96343547 | 96343597 |
| chr11 | 117598984 | 117599034 |
| chr11 | 117733877 | 117733927 |
| chr7 | 108973647 | 108973697 |
| chr7 | 116301055 | 116301105 |
| chr7 | 120006665 | 120006715 |
| chr7 | 122827023 | 122827073 |
| chr7 | 123040070 | 123040120 |
| chr7 | 125377973 | 125378023 |
| chr11 | 118183527 | 118183577 |
| chr7 | 132439665 | 132439715 |
| chr7 | 138006195 | 138006245 |
| chr7 | 138006019 | 138006069 |
| chr7 | 138941922 | 138941972 |
| chr7 | 140128601 | 140128651 |
| chr7 | 144315895 | 144315945 |
| chr7 | 146992429 | 146992479 |
| chr7 | 148223992 | 148224042 |
| chr7 | 151779333 | 151779383 |
| chr11 | 120624272 | 120624322 |
| chr8 | 3850282 | 3850332 |
| chr8 | 4004287 | 4004337 |
| chr8 | 4385082 | 4385132 |
| chr8 | 12431405 | 12431455 |
| chr8 | 12488148 | 12488198 |
| chr8 | 14300477 | 14300527 |
| chr8 | 17721016 | 17721066 |
| chr8 | 30767563 | 30767613 |
| chr8 | 37602685 | 37602735 |
| chr8 | 38469552 | 38469602 |
| chr8 | 39249213 | 39249263 |
| chr8 | 40482302 | 40482352 |
| chr8 | 53033718 | 53033768 |
| chr8 | 61118250 | 61118300 |
| chr8 | 62332846 | 62332896 |
| chr8 | 70084301 | 70084351 |
| chr8 | 81698805 | 81698855 |
| chr8 | 82389086 | 82389136 |
| chr8 | 85769127 | 85769177 |
| chr8 | 90436847 | 90436897 |
| chr11 | 127248997 | 127249047 |
| chr8 | 98469092 | 98469142 |
| chr8 | 98955033 | 98955083 |
| chr8 | 110267524 | 110267574 |
| chr8 | 110280018 | 110280068 |
| chr8 | 112562538 | 112562588 |
| chr8 | 116011722 | 116011772 |
| chr8 | 118544862 | 118544912 |
| chr8 | 118971229 | 118971279 |
| chr11 | 129173272 | 129173322 |
| chr11 | 129210517 | 129210567 |
| chr8 | 136232812 | 136232862 |
| chr8 | 142181817 | 142181867 |
| chr9 | 4638385 | 4638435 |
| chr9 | 5533223 | 5533273 |
| chr9 | 6306634 | 6306684 |
| chr9 | 12097039 | 12097089 |
| chr9 | 15404397 | 15404447 |
| chr9 | 20251522 | 20251572 |
| chr9 | 21835345 | 21835395 |
| chr9 | 24981607 | 24981657 |
| chr9 | 26417981 | 26418031 |
| chr9 | 27411019 | 27411069 |
| chr9 | 31121206 | 31121256 |
| chr9 | 33337370 | 33337420 |
| chr9 | 34203518 | 34203568 |
| chr9 | 35042210 | 35042260 |
| chr9 | 38011027 | 38011077 |
| chr9 | 81680958 | 81681008 |
| chr9 | 85597157 | 85597207 |
| chr10 | 20663720 | 20663770 |
| chr9 | 90658650 | 90658700 |
| chr9 | 90662332 | 90662382 |
| chr9 | 96879206 | 96879256 |
| chr9 | 97170774 | 97170824 |
| chr9 | 104148264 | 104148314 |
| chr9 | 106374455 | 106374505 |
| chr9 | 110492024 | 110492074 |
| chr9 | 116015475 | 116015525 |
| chr12 | 1123819 | 1123869 |
| chr9 | 129305795 | 129305845 |
| chr9 | 136050246 | 136050296 |
| chr12 | 5535567 | 5535617 |
| chr12 | 8396205 | 8396255 |
| chr12 | 10757152 | 10757202 |
| chr12 | 10757048 | 10757098 |
| chr12 | 15920356 | 15920406 |
| chr12 | 19819663 | 19819713 |
| chr12 | 29688948 | 29688998 |
| chr12 | 38626887 | 38626937 |
| chr12 | 38677964 | 38678014 |
| chr12 | 44201829 | 44201879 |
| chr10 | 25814368 | 25814418 |
| chr12 | 48294859 | 48294909 |
| chr12 | 49552247 | 49552297 |
| chr12 | 50287660 | 50287710 |
| chr10 | 26882533 | 26882583 |
| chr10 | 26941800 | 26941850 |
| chr10 | 27229406 | 27229456 |
| chr12 | 55954722 | 55954772 |
| chr12 | 56640809 | 56640859 |
| chr12 | 56640103 | 56640153 |
| chr12 | 67731903 | 67731953 |
| chr12 | 73436589 | 73436639 |
| chr12 | 75628125 | 75628175 |
| chr12 | 75633574 | 75633624 |
| chr12 | 76227516 | 76227566 |
| chr12 | 81967751 | 81967801 |
| chr12 | 87191902 | 87191952 |
| chr12 | 91209699 | 91209749 |
| chr12 | 91219318 | 91219368 |
| chr12 | 98749826 | 98749876 |
| chr12 | 103685990 | 103686040 |
| chr12 | 111185097 | 111185147 |
| chr12 | 114757353 | 114757403 |
| chr12 | 115609705 | 115609755 |
| chr12 | 115614175 | 115614225 |
| chr12 | 118295263 | 118295313 |
| chr12 | 121295132 | 121295182 |
| chr10 | 33452095 | 33452145 |
| chr12 | 125816420 | 125816470 |
| chr12 | 125839614 | 125839664 |
| chr12 | 126703409 | 126703459 |
| chr13 | 19539585 | 19539635 |
| chr13 | 20234551 | 20234601 |
| chr10 | 36415680 | 36415730 |
| chr13 | 25346667 | 25346717 |
| chr10 | 36957945 | 36957995 |
| chr13 | 30492540 | 30492590 |
| chr13 | 43618657 | 43618707 |
| chr13 | 45714995 | 45715045 |
| chr13 | 46293582 | 46293632 |
| chr13 | 50044511 | 50044561 |
| chr13 | 62943365 | 62943415 |
| chr13 | 63433129 | 63433179 |
| chr13 | 63434625 | 63434675 |
| chr13 | 68069865 | 68069915 |
| chr13 | 82764219 | 82764269 |
| chr13 | 83644821 | 83644871 |
| chr13 | 85351454 | 85351504 |
| chr13 | 85911036 | 85911086 |
| chr13 | 88282518 | 88282568 |
| chr13 | 90641224 | 90641274 |
| chr13 | 90648299 | 90648349 |
| chr13 | 96345693 | 96345743 |
| chr13 | 98538648 | 98538698 |
| chr13 | 100892110 | 100892160 |
| chr13 | 106000597 | 106000647 |
| chr13 | 111007495 | 111007545 |
| chr13 | 111988753 | 111988803 |
| chr14 | 18343219 | 18343269 |
| chr14 | 21662333 | 21662383 |
| chr14 | 21960461 | 21960511 |
| chr14 | 23670760 | 23670810 |
| chr14 | 27035994 | 27036044 |
| chr10 | 47032628 | 47032678 |
| chr14 | 40581301 | 40581351 |
| chr14 | 54905407 | 54905457 |
| chr14 | 56908720 | 56908770 |
| chr14 | 58364946 | 58364996 |
| chr14 | 61527384 | 61527434 |
| chr14 | 61795563 | 61795613 |
| chr14 | 66379074 | 66379124 |
| chr14 | 92909280 | 92909330 |
| chr14 | 97234246 | 97234296 |
| chr14 | 99333394 | 99333444 |
| chr10 | 58359349 | 58359399 |
| chr14 | 100632926 | 100632976 |
| chr14 | 102204340 | 102204390 |
| chr14 | 104926463 | 104926513 |
| chr15 | 21598705 | 21598755 |
| chr15 | 25973539 | 25973589 |
| chr15 | 29618531 | 29618581 |
| chr15 | 29677714 | 29677764 |
| chr15 | 30899447 | 30899497 |
| chr10 | 63625136 | 63625186 |
| chr15 | 43823338 | 43823388 |
| chr15 | 48182218 | 48182268 |
| chr15 | 53464704 | 53464754 |
| chr15 | 53468239 | 53468289 |
| chr15 | 53571562 | 53571612 |
| chr15 | 59436840 | 59436890 |
| chr10 | 66083679 | 66083729 |
| chr10 | 66569482 | 66569532 |
| chr15 | 65451859 | 65451909 |
| chr10 | 68333934 | 68333984 |
| chr15 | 70492597 | 70492647 |
| chr15 | 71230940 | 71230990 |
| chr15 | 90955162 | 90955212 |
| chr15 | 97764461 | 97764511 |
| chr15 | 99306726 | 99306776 |
| chr15 | 99867416 | 99867466 |
| chr16 | 5089906 | 5089956 |
| chr16 | 5325975 | 5326025 |
| chr10 | 71127189 | 71127239 |
| chr16 | 9429169 | 9429219 |
| chr16 | 10469693 | 10469743 |
| chr16 | 17608301 | 17608351 |
| chr16 | 58112778 | 58112828 |
| chr16 | 61161423 | 61161473 |
| chr16 | 70720060 | 70720110 |
| chr16 | 71409891 | 71409941 |
| chr16 | 75495898 | 75495948 |
| chr16 | 75641486 | 75641536 |
| chr16 | 81236619 | 81236669 |
| chr16 | 84245192 | 84245242 |
| chr16 | 86555580 | 86555630 |
| chr17 | 3482778 | 3482828 |
| chr17 | 25569914 | 25569964 |
| chr10 | 81547378 | 81547428 |

Table 15AG. Repeat Category L1PB4

| | | |
|---|---|---|
| chr17 | 38581903 | 38581953 |
| chr17 | 59444388 | 59444438 |
| chr17 | 79523759 | 79523809 |
| chr18 | 5123279 | 5123329 |
| chr18 | 5123109 | 5123159 |
| chr18 | 6021888 | 6021938 |
| chr18 | 8053452 | 8053502 |
| chr18 | 8716666 | 8716716 |
| chr18 | 12398727 | 12398777 |
| chr18 | 13232354 | 13232404 |
| chr18 | 19253945 | 19253995 |
| chr18 | 46759150 | 46759200 |
| chr18 | 48428682 | 48428732 |
| chr18 | 51382497 | 51382547 |
| chr18 | 51428880 | 51428930 |
| chr19 | 2210370 | 2210420 |
| chr19 | 6250321 | 6250371 |
| chr19 | 9001692 | 9001742 |
| chr19 | 17071360 | 17071410 |
| chr19 | 21046143 | 21046193 |
| chr19 | 37364866 | 37364916 |
| chr19 | 47728580 | 47728630 |
| chr19 | 50058264 | 50058314 |
| chr19 | 50058545 | 50058595 |
| chr19 | 50674763 | 50674813 |
| chr19 | 56099169 | 56099219 |
| chr19 | 62594913 | 62594963 |
| chr10 | 117933757 | 117933807 |
| chr1 | 21377715 | 21377765 |
| chr1 | 34472468 | 34472518 |
| chr1 | 42954681 | 42954731 |
| chr1 | 49807896 | 49807946 |
| chr1 | 52529338 | 52529388 |
| chr1 | 60241006 | 60241056 |
| chr1 | 88844852 | 88844902 |
| chr1 | 110909691 | 110909741 |
| chr1 | 114989101 | 114989151 |
| chr10 | 128063981 | 128064031 |
| chr1 | 156708152 | 156708202 |
| chr1 | 157988499 | 157988549 |
| chr1 | 187209369 | 187209419 |
| chr1 | 190258438 | 190258488 |
| chr1 | 190259855 | 190259905 |
| chr1 | 191357550 | 191357600 |
| chr1 | 193738662 | 193738712 |
| chr10 | 129923309 | 129923359 |
| chr1 | 207244589 | 207244639 |
| chr1 | 218286845 | 218286895 |
| chr1 | 224355671 | 224355721 |
| chr1 | 226871287 | 226871337 |
| chr1 | 228043173 | 228043223 |
| chr1 | 244588804 | 244588854 |
| chr1 | 244917095 | 244917145 |
| chr10 | 132663538 | 132663588 |
| chr20 | 10890743 | 10890793 |
| chr20 | 18318790 | 18318840 |
| chr20 | 18424532 | 18424582 |
| chr20 | 44832844 | 44832894 |
| chr20 | 53139999 | 53140049 |
| chr20 | 61691421 | 61691471 |
| chr21 | 18015648 | 18015698 |
| chr21 | 42659867 | 42659917 |
| chr21 | 42659758 | 42659808 |
| chrX | 3178226 | 3178276 |
| chrX | 18474015 | 18474065 |
| chrX | 18473733 | 18473783 |
| chrX | 23014562 | 23014612 |
| chrX | 32376662 | 32376712 |
| chrX | 37375718 | 37375768 |
| chrX | 40385785 | 40385835 |
| chrX | 46088912 | 46088962 |
| chrX | 46308210 | 46308260 |
| chrX | 47597451 | 47597501 |
| chrX | 53107455 | 53107505 |
| chrX | 53696224 | 53696274 |
| chrX | 94354445 | 94354495 |
| chrX | 95467954 | 95468004 |
| chrX | 98893479 | 98893529 |
| chrX | 105423659 | 105423709 |
| chr11 | 9951015 | 9951065 |
| chrX | 119456614 | 119456664 |
| chrX | 124153327 | 124153377 |
| chrX | 134910153 | 134910203 |
| chrX | 139236111 | 139236161 |
| chrX | 151771128 | 151771178 |
| chrY | 17492112 | 17492162 |
| chr2 | 644195 | 644245 |
| chr2 | 643851 | 643901 |
| chr2 | 2757802 | 2757852 |
| chr2 | 14120165 | 14120215 |
| chr2 | 14540219 | 14540269 |
| chr2 | 31199513 | 31199563 |
| chr2 | 33567398 | 33567448 |
| chr11 | 21508460 | 21508510 |
| chr2 | 52783190 | 52783240 |
| chr11 | 25518476 | 25518526 |
| chr2 | 109621520 | 109621570 |
| chr2 | 109621392 | 109621442 |
| chr2 | 133099963 | 133100013 |
| chr2 | 133802550 | 133802600 |
| chr2 | 158692205 | 158692255 |
| chr2 | 182557052 | 182557102 |
| chr2 | 184772276 | 184772326 |
| chr2 | 194748838 | 194748888 |
| chr11 | 41477614 | 41477664 |
| chr3 | 2816072 | 2816122 |
| chr3 | 11049480 | 11049530 |
| chr10 | 12392856 | 12392906 |
| chr3 | 34150538 | 34150588 |
| chr3 | 46056514 | 46056564 |
| chr3 | 58488560 | 58488610 |
| chr3 | 60415307 | 60415357 |
| chr3 | 82186034 | 82186084 |
| chr3 | 83263423 | 83263473 |
| chr10 | 12647366 | 12647416 |
| chr3 | 95072939 | 95072989 |
| chr3 | 123416540 | 123416590 |
| chr3 | 163945018 | 163945068 |
| chr3 | 164801229 | 164801279 |
| chr3 | 169182658 | 169182708 |
| chr3 | 174320387 | 174320437 |
| chr3 | 175559957 | 175560007 |
| chr3 | 175666880 | 175666930 |
| chr3 | 178049699 | 178049749 |
| chr3 | 197669612 | 197669662 |
| chr4 | 5872630 | 5872680 |
| chr4 | 17015719 | 17015769 |
| chr4 | 25058244 | 25058294 |
| chr4 | 25189586 | 25189636 |
| chr4 | 43912913 | 43912963 |
| chr4 | 48717693 | 48717743 |
| chr4 | 63012330 | 63012380 |
| chr4 | 75713557 | 75713607 |
| chr4 | 77431894 | 77431944 |
| chr4 | 94280544 | 94280594 |
| chr4 | 103703408 | 103703458 |
| chr4 | 108453105 | 108453155 |
| chr4 | 108454526 | 108454576 |
| chr4 | 109394444 | 109394494 |
| chr4 | 109394390 | 109394440 |
| chr4 | 110919510 | 110919560 |
| chr4 | 114339366 | 114339416 |
| chr4 | 115231995 | 115232045 |
| chr4 | 120251875 | 120251925 |
| chr4 | 121741119 | 121741169 |
| chr4 | 127793709 | 127793759 |
| chr4 | 143055189 | 143055239 |
| chr4 | 163248618 | 163248668 |
| chr4 | 187854494 | 187854544 |
| chr4 | 189190536 | 189190586 |
| chr4 | 191654000 | 191654050 |
| chr5 | 841636 | 841686 |
| chr5 | 14691226 | 14691276 |
| chr5 | 25880446 | 25880496 |
| chr5 | 28409845 | 28409895 |
| chr5 | 32034534 | 32034584 |
| chr5 | 40743327 | 40743377 |
| chr5 | 40959554 | 40959604 |
| chr5 | 54535046 | 54535096 |
| chr5 | 79582624 | 79582674 |
| chr5 | 100498637 | 100498687 |
| chr5 | 100543869 | 100543919 |
| chr5 | 115513112 | 115513162 |
| chr5 | 117849419 | 117849469 |
| chr5 | 126149928 | 126149978 |
| chr5 | 137045887 | 137045937 |
| chr5 | 161217176 | 161217226 |
| chr5 | 163072100 | 163072150 |
| chr5 | 168308824 | 168308874 |
| chr5 | 168768166 | 168768216 |
| chr5 | 180314715 | 180314765 |
| chr6 | 1249750 | 1249800 |
| chr6 | 3334186 | 3334236 |
| chr6 | 8828684 | 8828734 |
| chr6 | 17739865 | 17739915 |
| chr6 | 17946554 | 17946604 |
| chr11 | 79241342 | 79241392 |
| chr6 | 24183549 | 24183599 |
| chr6 | 27947165 | 27947215 |
| chr6 | 35880783 | 35880833 |
| chr11 | 85874704 | 85874754 |
| chr11 | 85874432 | 85874482 |
| chr6 | 42962299 | 42962349 |
| chr6 | 50657432 | 50657482 |
| chr6 | 73666963 | 73667013 |
| chr6 | 74417285 | 74417335 |
| chr6 | 76624564 | 76624614 |
| chr6 | 86756166 | 86756216 |
| chr6 | 105514483 | 105514533 |
| chr6 | 106406218 | 106406268 |
| chr6 | 109954469 | 109954519 |
| chr6 | 138777209 | 138777259 |
| chr6 | 144228786 | 144228836 |
| chr6 | 144232370 | 144232420 |
| chr6 | 152076156 | 152076206 |
| chr6 | 165999239 | 165999289 |
| chr6 | 169465958 | 169466008 |
| chr11 | 106770183 | 106770233 |
| chr7 | 576986 | 577036 |
| chr7 | 4385792 | 4385842 |
| chr7 | 7823217 | 7823267 |
| chr7 | 20372730 | 20372780 |
| chr7 | 20372671 | 20372721 |
| chr7 | 44368046 | 44368096 |
| chr7 | 46828289 | 46828339 |
| chr7 | 50448810 | 50448860 |
| chr7 | 53071229 | 53071279 |
| chr7 | 73127355 | 73127405 |
| chr7 | 80364532 | 80364582 |
| chr7 | 81783033 | 81783083 |
| chr7 | 86330017 | 86330067 |
| chr7 | 87838574 | 87838624 |
| chr7 | 89179566 | 89179616 |
| chr7 | 89667774 | 89667824 |
| chr7 | 97512378 | 97512428 |
| chr7 | 100648877 | 100648927 |
| chr7 | 114696302 | 114696352 |
| chr7 | 123144509 | 123144559 |
| chr11 | 118801906 | 118801956 |
| chr7 | 151976734 | 151976784 |
| chr7 | 157205799 | 157205849 |
| chr8 | 287326 | 287376 |
| chr8 | 1188363 | 1188413 |
| chr8 | 6625266 | 6625316 |
| chr11 | 121975793 | 121975843 |
| chr8 | 9181930 | 9181980 |
| chr8 | 16644864 | 16644914 |
| chr8 | 30072051 | 30072101 |
| chr8 | 30578547 | 30578597 |
| chr8 | 31690666 | 31690716 |
| chr8 | 41603520 | 41603570 |
| chr8 | 42660344 | 42660394 |
| chr8 | 43097624 | 43097674 |
| chr8 | 50825907 | 50825957 |
| chr8 | 60897970 | 60898020 |
| chr8 | 89654440 | 89654490 |
| chr8 | 93003796 | 93003846 |
| chr8 | 93408761 | 93408811 |
| chr8 | 103573744 | 103573794 |
| chr8 | 122662930 | 122662980 |
| chr8 | 126521512 | 126521562 |
| chr8 | 141657731 | 141657781 |
| chr9 | 4810019 | 4810069 |
| chr9 | 10354281 | 10354331 |
| chr9 | 15653889 | 15653939 |
| chr9 | 21607256 | 21607306 |
| chr9 | 22221116 | 22221166 |
| chr9 | 70360314 | 70360364 |
| chr9 | 72805129 | 72805179 |
| chr9 | 74881363 | 74881413 |
| chr9 | 86216046 | 86216096 |
| chr9 | 91042555 | 91042605 |
| chr9 | 98460886 | 98460936 |
| chr9 | 108500284 | 108500334 |
| chr9 | 110906105 | 110906155 |
| chr10 | 21610057 | 21610107 |
| chr9 | 130204370 | 130204420 |
| chr12 | 6183956 | 6184006 |
| chr12 | 6183449 | 6183499 |
| chr12 | 6403148 | 6403198 |
| chr12 | 10923227 | 10923277 |
| chr12 | 11281871 | 11281921 |
| chr12 | 11611573 | 11611623 |
| chr12 | 15583648 | 15583698 |
| chr12 | 43441639 | 43441689 |
| chr12 | 48704535 | 48704585 |
| chr12 | 54020468 | 54020518 |
| chr10 | 27941492 | 27941542 |
| chr12 | 68534826 | 68534876 |
| chr10 | 28709304 | 28709354 |
| chr12 | 72992702 | 72992752 |
| chr12 | 78987552 | 78987602 |
| chr12 | 80715804 | 80715854 |
| chr10 | 29328022 | 29328072 |
| chr10 | 29327879 | 29327929 |
| chr12 | 85160720 | 85160770 |
| chr12 | 104725158 | 104725208 |
| chr10 | 31500531 | 31500581 |
| chr12 | 121508468 | 121508518 |
| chr12 | 123410454 | 123410504 |
| chr12 | 123412669 | 123412719 |
| chr12 | 126625317 | 126625367 |
| chr12 | 127923689 | 127923739 |
| chr10 | 35223269 | 35223319 |
| chr12 | 130984052 | 130984102 |
| chr12 | 131515297 | 131515347 |
| chr12 | 131994853 | 131994903 |
| chr13 | 40596009 | 40596059 |
| chr13 | 46237015 | 46237065 |
| chr13 | 54027032 | 54027082 |
| chr13 | 72714829 | 72714879 |
| chr13 | 81132245 | 81132295 |
| chr13 | 102859181 | 102859231 |
| chr13 | 112688873 | 112688923 |
| chr14 | 27958240 | 27958290 |
| chr10 | 47852635 | 47852685 |
| chr14 | 41397411 | 41397461 |
| chr14 | 51819816 | 51819866 |
| chr14 | 60403169 | 60403219 |
| chr14 | 80792408 | 80792458 |
| chr14 | 82886135 | 82886185 |
| chr10 | 54460542 | 54460592 |
| chr14 | 90595430 | 90595480 |
| chr14 | 99571949 | 99571999 |
| chr15 | 21211794 | 21211844 |
| chr15 | 33581629 | 33581679 |
| chr15 | 35435131 | 35435181 |
| chr15 | 42251400 | 42251450 |
| chr15 | 52913382 | 52913432 |
| chr15 | 53211959 | 53212009 |
| chr15 | 68634766 | 68634816 |
| chr15 | 87806034 | 87806084 |
| chr15 | 91821777 | 91821827 |
| chr15 | 92402497 | 92402547 |
| chr16 | 2913096 | 2913146 |
| chr16 | 17453812 | 17453862 |
| chr16 | 19983582 | 19983632 |
| chr16 | 20176074 | 20176124 |
| chr16 | 27321563 | 27321613 |
| chr16 | 31622793 | 31622843 |
| chr16 | 31886326 | 31886376 |
| chr16 | 58473192 | 58473242 |
| chr16 | 68629764 | 68629814 |
| chr16 | 83165738 | 83165788 |
| chr16 | 88421930 | 88421980 |
| chr17 | 9470551 | 9470601 |
| chr17 | 12049626 | 12049676 |
| chr17 | 20978594 | 20978644 |
| chr17 | 29485880 | 29485930 |

Table 15AH. Repeat Category L1PBa1

| | | |
|---|---|---|
| chr18 | 52702152 | 52702202 |
| chr1 | 212053833 | 212053883 |
| chr1 | 242674122 | 242674172 |
| chr20 | 55578370 | 55578420 |
| chr22 | 22683906 | 22683956 |
| chrX | 39684884 | 39684934 |
| chrX | 41540414 | 41540464 |
| chrX | 73341126 | 73341176 |
| chrX | 76169416 | 76169466 |
| chrX | 120066320 | 120066370 |
| chrX | 141101318 | 141101368 |
| chr2 | 83719617 | 83719667 |
| chr2 | 129971588 | 129971638 |
| chr2 | 140255001 | 140255051 |
| chr11 | 36163651 | 36163701 |
| chr11 | 36163483 | 36163533 |
| chr4 | 9161394 | 9161444 |
| chr4 | 69438269 | 69438319 |
| chr4 | 78247579 | 78247629 |
| chr4 | 155359931 | 155359981 |
| chr4 | 169513106 | 169513156 |
| chr4 | 184594709 | 184594759 |
| chr4 | 189979861 | 189979911 |
| chr4 | 189979591 | 189979641 |
| chr5 | 53150156 | 53150206 |
| chr5 | 56411882 | 56411932 |
| chr11 | 71280157 | 71280207 |
| chr6 | 104228705 | 104228755 |
| chr6 | 115049679 | 115049729 |
| chr6 | 166458144 | 166458194 |
| chr7 | 156082285 | 156082335 |
| chr7 | 156083947 | 156083997 |
| chr11 | 121963934 | 121963984 |
| chr8 | 122986543 | 122986593 |
| chr8 | 122987406 | 122987456 |
| chr9 | 29750824 | 29750874 |
| chr9 | 102868749 | 102868799 |
| chr12 | 15918590 | 15918640 |
| chr12 | 17198287 | 17198337 |
| chr12 | 19819663 | 19819713 |
| chr12 | 20136289 | 20136339 |
| chr12 | 31070458 | 31070508 |
| chr10 | 38912633 | 38912683 |
| chr14 | 65174788 | 65174838 |
| chr15 | 33365732 | 33365782 |
| chr15 | 68737773 | 68737823 |
| chr17 | 6957302 | 6957352 |
| chr17 | 11067285 | 11067335 |

Table 15AI. Repeat Category LTR10B

| | | |
|---|---|---|
| chr17 | 75761332 | 75761382 |
| chr17 | 75767813 | 75767863 |
| chr18 | 42702251 | 42702301 |
| chr18 | 42709465 | 42709515 |
| chr19 | 11786268 | 11786318 |
| chr19 | 11787190 | 11787240 |
| chr19 | 11787333 | 11787383 |
| chr19 | 19838683 | 19838733 |
| chr19 | 19873351 | 19873401 |
| chr19 | 19873136 | 19873186 |
| chr19 | 20050616 | 20050666 |
| chr19 | 20050315 | 20050365 |
| chr19 | 20140106 | 20140156 |
| chr19 | 20547126 | 20547176 |
| chr19 | 20635101 | 20635151 |
| chr19 | 20635493 | 20635543 |
| chr19 | 23546170 | 23546220 |
| chr1 | 3257777 | 3257827 |
| chr1 | 96839370 | 96839420 |
| chr1 | 238141441 | 238141491 |
| chr20 | 1443812 | 1443862 |
| chr20 | 48584794 | 48584844 |
| chr20 | 48588906 | 48588956 |
| chr20 | 48588684 | 48588734 |
| chr2 | 27362976 | 27363026 |
| chr3 | 11267068 | 11267118 |
| chr3 | 121529902 | 121529952 |
| chr3 | 177972676 | 177972726 |
| chr6 | 27255487 | 27255537 |
| chr6 | 27949213 | 27949263 |
| chr6 | 35581077 | 35581127 |
| chr6 | 118945482 | 118945532 |
| chr7 | 137289407 | 137289457 |
| chr8 | 15968483 | 15968533 |
| chr9 | 131183772 | 131183822 |
| chr9 | 131189678 | 131189728 |
| chr12 | 17127233 | 17127283 |
| chr15 | 78011415 | 78011465 |
| chr16 | 30145781 | 30145831 |
| chr16 | 30146412 | 30146462 |
| chr16 | 30147966 | 30148016 |
| chr16 | 30150310 | 30150360 |
| chr16 | 71874243 | 71874293 |

Table 15AJ. Repeat Category LTR10B1

| | | |
|---|---|---|
| chr17 | 75761332 | 75761382 |
| chr17 | 75767813 | 75767863 |
| chr18 | 42702251 | 42702301 |
| chr18 | 42709465 | 42709515 |
| chr19 | 11786268 | 11786318 |
| chr19 | 11787190 | 11787240 |
| chr19 | 11787333 | 11787383 |
| chr19 | 19838683 | 19838733 |
| chr19 | 19873351 | 19873401 |
| chr19 | 19873136 | 19873186 |
| chr19 | 20050616 | 20050666 |
| chr19 | 20050315 | 20050365 |
| chr19 | 20140106 | 20140156 |
| chr19 | 20547126 | 20547176 |
| chr19 | 20635101 | 20635151 |
| chr19 | 20635493 | 20635543 |
| chr19 | 23546170 | 23546220 |
| chr1 | 3257777 | 3257827 |
| chr1 | 238141441 | 238141491 |
| chr20 | 48584794 | 48584844 |
| chr20 | 48588906 | 48588956 |
| chr20 | 48588684 | 48588734 |
| chr2 | 27362976 | 27363026 |
| chr3 | 11267068 | 11267118 |
| chr3 | 121529902 | 121529952 |
| chr6 | 27255487 | 27255537 |
| chr6 | 27949213 | 27949263 |
| chr6 | 118945482 | 118945532 |
| chr7 | 137289407 | 137289457 |
| chr12 | 17127233 | 17127283 |
| chr15 | 78011415 | 78011465 |
| chr16 | 30145781 | 30145831 |
| chr16 | 30146412 | 30146462 |
| chr16 | 30147966 | 30148016 |
| chr16 | 30150310 | 30150360 |

Table 15AK. Repeat Category LTR14

| | | |
|---|---|---|
| chr10 | 82134312 | 82134362 |
| chr17 | 51513647 | 51513697 |
| chr10 | 88445843 | 88445893 |
| chr17 | 73616199 | 73616249 |
| chr17 | 73623410 | 73623460 |
| chr17 | 80813179 | 80813229 |
| chr18 | 5237408 | 5237458 |
| chr18 | 25876242 | 25876292 |
| chr10 | 98211645 | 98211695 |
| chr18 | 75693206 | 75693256 |
| chr19 | 7762730 | 7762780 |
| chr19 | 7772243 | 7772293 |
| chr19 | 21070381 | 21070431 |
| chr19 | 23851270 | 23851320 |
| chr19 | 24145792 | 24145842 |
| chr19 | 41819158 | 41819208 |
| chr19 | 42247649 | 42247699 |
| chr19 | 43521410 | 43521460 |
| chr19 | 43521721 | 43521771 |
| chr19 | 56426247 | 56426297 |
| chr19 | 57661067 | 57661117 |
| chr19 | 63107915 | 63107965 |
| chr1 | 17009439 | 17009489 |
| chr1 | 62014127 | 62014177 |
| chr1 | 75242614 | 75242664 |
| chr1 | 152885526 | 152885576 |
| chr1 | 192979207 | 192979257 |
| chr1 | 192980939 | 192980989 |
| chr1 | 222953314 | 222953364 |
| chr1 | 222962980 | 222963030 |
| chr20 | 2784928 | 2784978 |
| chr21 | 43974691 | 43974741 |
| chr21 | 44015635 | 44015685 |
| chr22 | 29396210 | 29396260 |
| chr22 | 29400220 | 29400270 |
| chr22 | 39367811 | 39367861 |
| chr22 | 43861367 | 43861417 |
| chr22 | 43861504 | 43861554 |
| chrX | 10413820 | 10413870 |
| chr11 | 6998874 | 6998924 |
| chrX | 72745716 | 72745766 |
| chrX | 81092602 | 81092652 |
| chrX | 99210080 | 99210130 |
| chrX | 116188297 | 116188347 |
| chrX | 152294009 | 152294059 |
| chrY | 7697268 | 7697318 |
| chrY | 20224249 | 20224299 |
| chrY | 22458742 | 22458792 |
| chrY | 22458853 | 22458903 |
| chr11 | 24215940 | 24215990 |
| chr2 | 82225138 | 82225188 |
| chr2 | 97979228 | 97979278 |
| chr2 | 98203135 | 98203185 |
| chr2 | 125877021 | 125877071 |
| chr2 | 139829274 | 139829324 |
| chr2 | 164144919 | 164144969 |
| chr2 | 181124049 | 181124099 |
| chr2 | 181128659 | 181128709 |
| chr2 | 193555207 | 193555257 |
| chr11 | 41151076 | 41151126 |
| chr2 | 211293321 | 211293371 |
| chr2 | 228806829 | 228806879 |
| chr3 | 58667893 | 58667943 |
| chr3 | 81299971 | 81300021 |
| chr3 | 129868624 | 129868674 |
| chr3 | 129960517 | 129960567 |
| chr3 | 161283877 | 161283927 |
| chr3 | 173758124 | 173758174 |
| chr11 | 62854917 | 62854967 |
| chr4 | 1673290 | 1673340 |
| chr4 | 8636228 | 8636278 |
| chr4 | 84945480 | 84945530 |
| chr4 | 167773271 | 167773321 |
| chr4 | 182747168 | 182747218 |
| chr5 | 23733108 | 23733158 |
| chr5 | 24353855 | 24353905 |
| chr5 | 24796617 | 24796667 |
| chr5 | 31439340 | 31439390 |
| chr5 | 31444280 | 31444330 |
| chr5 | 141497348 | 141497398 |
| chr5 | 178214377 | 178214427 |
| chr5 | 178283380 | 178283430 |
| chr5 | 178449849 | 178449899 |
| chr6 | 27263871 | 27263921 |
| chr6 | 32062308 | 32062358 |
| chr6 | 48888931 | 48888981 |
| chr11 | 87323330 | 87323380 |
| chr6 | 79559788 | 79559838 |
| chr6 | 83003392 | 83003442 |
| chr6 | 108357622 | 108357672 |
| chr6 | 166668349 | 166668399 |
| chr6 | 169241030 | 169241080 |
| chr7 | 8801179 | 8801229 |
| chr7 | 14294040 | 14294090 |
| chr7 | 47985412 | 47985462 |
| chr7 | 82505201 | 82505251 |
| chr7 | 108824900 | 108824950 |
| chr11 | 120645173 | 120645223 |
| chr11 | 120689921 | 120689971 |
| chr8 | 42964621 | 42964671 |
| chr9 | 22221116 | 22221166 |
| chr9 | 28049344 | 28049394 |
| chr9 | 29649846 | 29649896 |
| chr9 | 29770178 | 29770228 |
| chr9 | 34529880 | 34529930 |
| chr9 | 99269305 | 99269355 |
| chr9 | 118635803 | 118635853 |
| chr12 | 10180831 | 10180881 |
| chr12 | 17516688 | 17516738 |
| chr12 | 36824555 | 36824605 |
| chr12 | 45585167 | 45585217 |
| chr12 | 47191786 | 47191836 |
| chr12 | 47191649 | 47191699 |
| chr13 | 36691902 | 36691952 |
| chr13 | 63081280 | 63081330 |
| chr13 | 91390124 | 91390174 |
| chr13 | 111180394 | 111180444 |
| chr14 | 46470996 | 46471046 |
| chr14 | 78163281 | 78163331 |
| chr15 | 43242720 | 43242770 |
| chr15 | 56122109 | 56122159 |
| chr15 | 76630404 | 76630454 |
| chr16 | 20694489 | 20694539 |
| chr16 | 63569578 | 63569628 |
| chr10 | 74766673 | 74766723 |
| chr17 | 8160981 | 8161031 |
| chr17 | 8160859 | 8160909 |

Table 15AL. Repeat Category LTR14B

| | | |
|---|---|---|
| chr17 | 80813179 | 80813229 |
| chr18 | 5237408 | 5237458 |
| chr18 | 25876242 | 25876292 |
| chr19 | 21070381 | 21070431 |
| chr19 | 24145792 | 24145842 |
| chr19 | 42247649 | 42247699 |
| chr19 | 43521410 | 43521460 |
| chr19 | 43521721 | 43521771 |
| chr19 | 56426247 | 56426297 |
| chr19 | 63107915 | 63107965 |
| chr1 | 222953314 | 222953364 |
| chr1 | 222962980 | 222963030 |
| chr22 | 39367811 | 39367861 |
| chr11 | 6998874 | 6998924 |
| chrX | 72745716 | 72745766 |
| chrX | 152294009 | 152294059 |
| chr11 | 24215940 | 24215990 |
| chr2 | 97979228 | 97979278 |
| chr2 | 164144919 | 164144969 |
| chr2 | 181124049 | 181124099 |
| chr2 | 181128659 | 181128709 |
| chr11 | 41151076 | 41151126 |
| chr2 | 211293321 | 211293371 |
| chr2 | 228806829 | 228806879 |
| chr3 | 129960517 | 129960567 |
| chr3 | 161283877 | 161283927 |
| chr3 | 173758124 | 173758174 |
| chr4 | 182747168 | 182747218 |
| chr5 | 23733108 | 23733158 |
| chr5 | 24796617 | 24796667 |
| chr5 | 31439340 | 31439390 |
| chr5 | 31444280 | 31444330 |
| chr5 | 178214377 | 178214427 |
| chr5 | 178283380 | 178283430 |
| chr5 | 178449849 | 178449899 |
| chr6 | 79559788 | 79559838 |
| chr6 | 166668349 | 166668399 |
| chr7 | 8801179 | 8801229 |
| chr7 | 82505201 | 82505251 |
| chr7 | 108824900 | 108824950 |
| chr11 | 120645173 | 120645223 |
| chr11 | 120689921 | 120689971 |
| chr9 | 22221116 | 22221166 |
| chr9 | 28049344 | 28049394 |
| chr9 | 29649846 | 29649896 |
| chr9 | 29770178 | 29770228 |
| chr9 | 99269305 | 99269355 |
| chr9 | 118635803 | 118635853 |
| chr12 | 10180831 | 10180881 |
| chr12 | 17516688 | 17516738 |
| chr12 | 45585167 | 45585217 |
| chr12 | 47191786 | 47191836 |
| chr12 | 47191649 | 47191699 |
| chr13 | 63081280 | 63081330 |
| chr13 | 91390124 | 91390174 |
| chr13 | 111180394 | 111180444 |
| chr15 | 56122109 | 56122159 |
| chr15 | 76630404 | 76630454 |
| chr16 | 20694489 | 20694539 |

Table 15AM. Repeat Category LTR14C

| | | |
|---|---|---|
| chr17 | 51513647 | 51513697 |
| chr19 | 7762730 | 7762780 |
| chr19 | 7772243 | 7772293 |
| chr1 | 192979207 | 192979257 |
| chr1 | 192980939 | 192980989 |
| chr20 | 2784928 | 2784978 |
| chrX | 10413820 | 10413870 |
| chrY | 7697268 | 7697318 |
| chrY | 20224249 | 20224299 |
| chrY | 22458742 | 22458792 |
| chrY | 22458853 | 22458903 |
| chr2 | 82225138 | 82225188 |
| chr2 | 98203135 | 98203185 |
| chr2 | 125877021 | 125877071 |
| chr2 | 139829274 | 139829324 |
| chr2 | 193555207 | 193555257 |
| chr3 | 81299971 | 81300021 |
| chr4 | 84945480 | 84945530 |
| chr4 | 167773271 | 167773321 |
| chr6 | 48888931 | 48888981 |
| chr6 | 83003392 | 83003442 |
| chr6 | 169241030 | 169241080 |
| chr7 | 14294040 | 14294090 |
| chr7 | 47985412 | 47985462 |
| chr8 | 42964621 | 42964671 |
| chr12 | 36824555 | 36824605 |
| chr13 | 36691902 | 36691952 |
| chr14 | 46470996 | 46471046 |
| chr15 | 43242720 | 43242770 |
| chr16 | 63569578 | 63569628 |

Table 15AN. Repeat Category LTR17

| | | |
|---|---|---|
| chr17 | 45658618 | 45658668 |
| chr10 | 83255290 | 83255340 |
| chr17 | 48951322 | 48951372 |
| chr17 | 53570257 | 53570307 |
| chr10 | 85949723 | 85949773 |
| chr10 | 85989986 | 85990036 |
| chr10 | 88036489 | 88036539 |
| chr17 | 69170841 | 69170891 |
| chr17 | 71231705 | 71231755 |
| chr18 | 4682178 | 4682228 |
| chr18 | 25231092 | 25231142 |
| chr18 | 35191349 | 35191399 |
| chr18 | 41996588 | 41996638 |
| chr18 | 50165618 | 50165668 |
| chr18 | 53551447 | 53551497 |
| chr10 | 98714916 | 98714966 |
| chr18 | 71777429 | 71777479 |
| chr19 | 20072902 | 20072952 |
| chr19 | 43872618 | 43872668 |
| chr19 | 43872736 | 43872786 |
| chr19 | 43873380 | 43873430 |
| chr19 | 45166413 | 45166463 |
| chr19 | 59627585 | 59627635 |
| chr10 | 120023871 | 120023921 |
| chr1 | 54746573 | 54746623 |
| chr1 | 54754695 | 54754745 |
| chr1 | 55619664 | 55619714 |
| chr1 | 66983805 | 66983855 |
| chr1 | 68227690 | 68227740 |
| chr1 | 68227945 | 68227995 |
| chr1 | 80480933 | 80480983 |
| chr1 | 83627454 | 83627504 |
| chr1 | 88857722 | 88857772 |
| chr1 | 119071816 | 119071866 |
| chr1 | 175020867 | 175020917 |
| chr1 | 175350090 | 175350140 |
| chr1 | 204033835 | 204033885 |
| chr1 | 204998449 | 204998499 |
| chr1 | 218781096 | 218781146 |
| chr1 | 224787299 | 224787349 |
| chr1 | 226039857 | 226039907 |
| chr20 | 25567242 | 25567292 |
| chr20 | 38012756 | 38012806 |
| chr20 | 40900314 | 40900364 |
| chr21 | 19053961 | 19054011 |
| chr21 | 19971628 | 19971678 |
| chr21 | 20342865 | 20342915 |
| chr21 | 22654305 | 22654355 |
| chr21 | 25412957 | 25413007 |
| chr21 | 27149320 | 27149370 |
| chr21 | 40038137 | 40038187 |
| chr21 | 40038539 | 40038589 |
| chr21 | 41715562 | 41715612 |
| chr22 | 27482081 | 27482131 |
| chr22 | 32578459 | 32578509 |
| chrX | 7082806 | 7082856 |
| chrX | 20351787 | 20351837 |
| chrX | 26135420 | 26135470 |
| chrX | 27700607 | 27700657 |
| chrX | 27702543 | 27702593 |
| chrX | 67534924 | 67534974 |
| chrX | 70300047 | 70300097 |
| chr11 | 9083663 | 9083713 |
| chrX | 87761833 | 87761883 |
| chrX | 88978739 | 88978789 |
| chrX | 92008451 | 92008501 |
| chrX | 101123629 | 101123679 |
| chrX | 101142058 | 101142108 |
| chrX | 103663052 | 103663102 |
| chrX | 105066921 | 105066971 |
| chrX | 111554428 | 111554478 |
| chrX | 111903857 | 111903907 |
| chrX | 117197325 | 117197375 |
| chrX | 119188270 | 119188320 |
| chrX | 125655802 | 125655852 |
| chrX | 130259762 | 130259812 |
| chrX | 135701274 | 135701324 |
| chrX | 144770216 | 144770266 |
| chrX | 146693725 | 146693775 |
| chrY | 4098322 | 4098372 |
| chrY | 14925140 | 14925190 |
| chr2 | 150370 | 150420 |
| chr2 | 17505085 | 17505135 |
| chr2 | 26455967 | 26456017 |
| chr2 | 30466759 | 30466809 |
| chr2 | 30713843 | 30713893 |
| chr2 | 30717341 | 30717391 |
| chr2 | 31482499 | 31482549 |
| chr2 | 33857360 | 33857410 |
| chr2 | 34932350 | 34932400 |
| chr2 | 43033625 | 43033675 |
| chr2 | 53958434 | 53958484 |
| chr11 | 22303684 | 22303734 |
| chr2 | 58477538 | 58477588 |
| chr11 | 23250755 | 23250805 |
| chr11 | 23427094 | 23427144 |
| chr11 | 25952001 | 25952051 |
| chr11 | 26576805 | 26576855 |
| chr11 | 26582972 | 26583022 |
| chr2 | 76073721 | 76073771 |
| chr2 | 78748363 | 78748413 |
| chr2 | 81073326 | 81073376 |
| chr2 | 96367996 | 96368046 |
| chr2 | 106453087 | 106453137 |
| chr2 | 112897177 | 112897227 |
| chr2 | 139241831 | 139241881 |
| chr2 | 166027869 | 166027919 |
| chr2 | 172618972 | 172619022 |
| chr11 | 36311198 | 36311248 |
| chr2 | 183620343 | 183620393 |
| chr11 | 38489858 | 38489908 |
| chr2 | 189251849 | 189251899 |
| chr2 | 196407284 | 196407334 |
| chr2 | 207007232 | 207007282 |
| chr2 | 210373857 | 210373907 |
| chr2 | 239709445 | 239709495 |
| chr3 | 19892375 | 19892425 |
| chr3 | 38291516 | 38291566 |
| chr3 | 38298973 | 38299023 |
| chr3 | 43487872 | 43487922 |
| chr3 | 48338101 | 48338151 |
| chr3 | 58382900 | 58382950 |
| chr3 | 70599576 | 70599626 |
| chr3 | 70600051 | 70600101 |
| chr3 | 75609014 | 75609064 |
| chr3 | 78314305 | 78314355 |
| chr3 | 83485658 | 83485708 |
| chr3 | 86382005 | 86382055 |
| chr3 | 87915848 | 87915898 |
| chr3 | 88856901 | 88856951 |
| chr3 | 95004507 | 95004557 |
| chr3 | 103772202 | 103772252 |
| chr3 | 107006203 | 107006253 |
| chr3 | 107526849 | 107526899 |
| chr11 | 59910876 | 59910926 |
| chr3 | 112537135 | 112537185 |
| chr3 | 113830487 | 113830537 |
| chr3 | 144182411 | 144182461 |
| chr3 | 147055456 | 147055506 |
| chr3 | 151893743 | 151893793 |
| chr3 | 163705686 | 163705736 |
| chr3 | 180093425 | 180093475 |
| chr3 | 186303767 | 186303817 |
| chr3 | 189977269 | 189977319 |
| chr3 | 192698140 | 192698190 |
| chr4 | 5341069 | 5341119 |
| chr4 | 18950631 | 18950681 |
| chr4 | 42075729 | 42075779 |
| chr4 | 42203712 | 42203762 |
| chr4 | 42209456 | 42209506 |
| chr4 | 47468563 | 47468613 |
| chr4 | 59278271 | 59278321 |
| chr4 | 63391671 | 63391721 |
| chr4 | 63395615 | 63395665 |
| chr4 | 74257863 | 74257913 |
| chr4 | 83859199 | 83859249 |
| chr4 | 87336318 | 87336368 |
| chr4 | 96072171 | 96072221 |
| chr4 | 97151285 | 97151335 |
| chr4 | 99869248 | 99869298 |
| chr4 | 100489317 | 100489367 |
| chr4 | 111061728 | 111061778 |
| chr4 | 111663860 | 111663910 |
| chr4 | 115424467 | 115424517 |
| chr4 | 127743716 | 127743766 |
| chr4 | 133470625 | 133470675 |
| chr4 | 136752614 | 136752664 |
| chr4 | 139112101 | 139112151 |
| chr4 | 155691275 | 155691325 |
| chr4 | 163603219 | 163603269 |
| chr4 | 173246525 | 173246575 |
| chr4 | 179025323 | 179025373 |
| chr4 | 189382622 | 189382672 |
| chr5 | 12713567 | 12713617 |
| chr5 | 14901464 | 14901514 |
| chr5 | 18778859 | 18778909 |
| chr5 | 18845879 | 18845929 |
| chr5 | 21660607 | 21660657 |
| chr5 | 23456994 | 23457044 |
| chr5 | 26706083 | 26706133 |
| chr5 | 27861787 | 27861837 |
| chr5 | 35553812 | 35553862 |
| chr5 | 36378746 | 36378796 |
| chr5 | 55499292 | 55499342 |
| chr5 | 58227113 | 58227163 |
| chr5 | 59977841 | 59977891 |
| chr5 | 60107317 | 60107367 |
| chr11 | 70330819 | 70330869 |
| chr5 | 64342598 | 64342648 |
| chr5 | 84545287 | 84545337 |
| chr11 | 71591751 | 71591801 |
| chr5 | 111855398 | 111855448 |
| chr5 | 115230822 | 115230872 |
| chr5 | 119163301 | 119163351 |
| chr5 | 143453581 | 143453631 |
| chr5 | 143706012 | 143706062 |
| chr5 | 157375317 | 157375367 |
| chr5 | 172313520 | 172313570 |
| chr6 | 11757234 | 11757284 |
| chr6 | 14176842 | 14176892 |
| chr11 | 80137036 | 80137086 |
| chr6 | 24790853 | 24790903 |
| chr6 | 29634362 | 29634412 |
| chr6 | 38440742 | 38440792 |
| chr6 | 52833260 | 52833310 |
| chr6 | 57741091 | 57741141 |
| chr6 | 65369549 | 65369599 |
| chr6 | 65710923 | 65710973 |
| chr6 | 67907146 | 67907196 |
| chr6 | 82043229 | 82043279 |
| chr6 | 82056775 | 82056825 |
| chr6 | 85417365 | 85417415 |
| chr6 | 89126628 | 89126678 |
| chr6 | 103415113 | 103415163 |
| chr6 | 106729336 | 106729386 |
| chr6 | 110246491 | 110246541 |
| chr6 | 111497975 | 111498025 |
| chr6 | 119054664 | 119054714 |
| chr6 | 119343390 | 119343440 |
| chr6 | 135549736 | 135549786 |
| chr6 | 143389193 | 143389243 |
| chr6 | 144423057 | 144423107 |
| chr6 | 158800404 | 158800454 |
| chr11 | 103053217 | 103053267 |
| chr6 | 167214310 | 167214360 |
| chr11 | 107198009 | 107198059 |
| chr11 | 107389829 | 107389879 |
| chr11 | 109736688 | 109736738 |
| chr7 | 16849885 | 16849935 |
| chr7 | 24913330 | 24913380 |
| chr7 | 27057802 | 27057852 |
| chr7 | 39953344 | 39953394 |
| chr7 | 42991201 | 42991251 |
| chr7 | 46317127 | 46317177 |
| chr11 | 113372834 | 113372884 |
| chr7 | 47824574 | 47824624 |
| chr7 | 68360015 | 68360065 |
| chr7 | 82916121 | 82916171 |
| chr7 | 91710219 | 91710269 |
| chr7 | 91718779 | 91718829 |
| chr7 | 91719568 | 91719618 |
| chr7 | 95600042 | 95600092 |
| chr7 | 96315498 | 96315548 |
| chr7 | 106173732 | 106173782 |
| chr7 | 107538795 | 107538845 |
| chr7 | 108652794 | 108652844 |
| chr7 | 111421108 | 111421158 |
| chr7 | 113580624 | 113580674 |
| chr7 | 121371968 | 121372018 |
| chr7 | 133007927 | 133007977 |
| chr7 | 148765802 | 148765852 |
| chr7 | 151777545 | 151777595 |
| chr8 | 15184535 | 15184585 |
| chr8 | 18142411 | 18142461 |
| chr8 | 20684749 | 20684799 |
| chr8 | 38742904 | 38742954 |
| chr11 | 124498634 | 124498684 |
| chr8 | 51906806 | 51906856 |
| chr8 | 62127752 | 62127802 |
| chr8 | 98743318 | 98743368 |
| chr8 | 105437778 | 105437828 |
| chr8 | 107808106 | 107808156 |
| chr8 | 124380690 | 124380740 |
| chr8 | 132312046 | 132312096 |
| chr9 | 2254585 | 2254635 |
| chr9 | 21717768 | 21717818 |
| chr9 | 22867411 | 22867461 |
| chr9 | 36255164 | 36255214 |
| chr9 | 74541634 | 74541684 |
| chr9 | 77984123 | 77984173 |
| chr9 | 77983950 | 77984000 |
| chr9 | 100578866 | 100578916 |
| chr12 | 8805684 | 8805734 |
| chr12 | 15139554 | 15139604 |
| chr12 | 19418547 | 19418597 |
| chr10 | 23594763 | 23594813 |
| chr10 | 23806290 | 23806340 |
| chr12 | 32319543 | 32319593 |
| chr12 | 45933103 | 45933153 |
| chr12 | 49582459 | 49582509 |
| chr12 | 57532706 | 57532756 |
| chr12 | 57538843 | 57538893 |
| chr12 | 62999997 | 63000047 |
| chr12 | 71506337 | 71506387 |
| chr12 | 75897636 | 75897686 |
| chr12 | 77355926 | 77355976 |
| chr12 | 83690491 | 83690541 |
| chr12 | 101052072 | 101052122 |
| chr12 | 122396379 | 122396429 |
| chr12 | 122943625 | 122943675 |
| chr10 | 33884906 | 33884956 |
| chr12 | 130852526 | 130852576 |
| chr13 | 41418998 | 41419048 |
| chr13 | 53433275 | 53433325 |
| chr13 | 54418429 | 54418479 |
| chr13 | 58869201 | 58869251 |
| chr13 | 59930094 | 59930144 |
| chr13 | 61877697 | 61877747 |
| chr13 | 62898016 | 62898066 |
| chr13 | 64120480 | 64120530 |
| chr13 | 76431614 | 76431664 |
| chr13 | 78564562 | 78564612 |
| chr13 | 80829823 | 80829873 |
| chr13 | 81811722 | 81811772 |
| chr13 | 81957048 | 81957098 |
| chr13 | 87962344 | 87962394 |
| chr13 | 91557394 | 91557444 |
| chr13 | 92653272 | 92653322 |
| chr13 | 109859683 | 109859733 |
| chr14 | 24719699 | 24719749 |
| chr14 | 35003289 | 35003339 |
| chr14 | 47206019 | 47206069 |
| chr14 | 51565171 | 51565221 |
| chr14 | 51819816 | 51819866 |
| chr14 | 64381891 | 64381941 |
| chr14 | 70228566 | 70228616 |
| chr14 | 78860594 | 78860644 |
| chr14 | 81682161 | 81682211 |
| chr10 | 54764210 | 54764260 |
| chr10 | 57455931 | 57455981 |
| chr10 | 57456545 | 57456595 |
| chr14 | 104380402 | 104380452 |
| chr15 | 23449543 | 23449593 |
| chr15 | 27532980 | 27533030 |
| chr10 | 62138094 | 62138144 |
| chr10 | 62143801 | 62143851 |
| chr15 | 33053237 | 33053287 |
| chr15 | 43555536 | 43555586 |
| chr15 | 49269282 | 49269332 |
| chr15 | 49269612 | 49269662 |
| chr15 | 49663752 | 49663802 |
| chr15 | 50447205 | 50447255 |
| chr15 | 51634570 | 51634620 |
| chr10 | 64768945 | 64768995 |
| chr15 | 53320135 | 53320185 |
| chr10 | 65149622 | 65149672 |
| chr15 | 64984973 | 64985023 |
| chr15 | 76247442 | 76247492 |
| chr15 | 90571221 | 90571271 |
| chr15 | 92430725 | 92430775 |
| chr15 | 92924343 | 92924393 |
| chr16 | 57011286 | 57011336 |
| chr16 | 82604893 | 82604943 |

Table 15AO. Repeat Category LTR19A

| | | |
|---|---|---|
| chr17 | 47677535 | 47677585 |
| chr17 | 79909235 | 79909285 |
| chr19 | 4938834 | 4938884 |
| chr19 | 8773925 | 8773975 |
| chr19 | 12201376 | 12201426 |
| chr19 | 23147388 | 23147438 |
| chr1 | 2391717 | 2391767 |
| chr1 | 8865108 | 8865158 |
| chr1 | 11845292 | 11845342 |
| chr1 | 45202748 | 45202798 |
| chr10 | 132565859 | 132565909 |
| chr21 | 36355142 | 36355192 |
| chr22 | 22927121 | 22927171 |
| chrX | 139379742 | 139379792 |
| chrX | 140619233 | 140619283 |
| chrY | 3274581 | 3274631 |
| chr2 | 10364955 | 10365005 |
| chr2 | 53920964 | 53921014 |
| chr2 | 101494934 | 101494984 |
| chr2 | 239400787 | 239400837 |
| chr11 | 62389984 | 62390034 |
| chr4 | 33149821 | 33149871 |
| chr4 | 33152981 | 33153031 |
| chr4 | 33154101 | 33154151 |
| chr4 | 37378924 | 37378974 |
| chr4 | 134978789 | 134978839 |
| chr7 | 54835488 | 54835538 |
| chr8 | 2077147 | 2077197 |
| chr8 | 15402213 | 15402263 |
| chr9 | 127728957 | 127729007 |
| chr9 | 127729901 | 127729951 |
| chr9 | 127730909 | 127730959 |
| chr9 | 127732340 | 127732390 |
| chr12 | 33027763 | 33027813 |
| chr10 | 42159726 | 42159776 |
| chr13 | 112532205 | 112532255 |
| chr14 | 58555302 | 58555352 |

Table 15AP. Repeat Category LTR1B

| | | |
|---|---|---|
| chr18 | 31758976 | 31759026 |
| chr19 | 48162548 | 48162598 |
| chr1 | 4131184 | 4131234 |
| chr1 | 35099514 | 35099564 |
| chr1 | 38691569 | 38691619 |
| chr1 | 70542234 | 70542284 |
| chr1 | 159135644 | 159135694 |
| chr1 | 173665588 | 173665638 |
| chr1 | 193908027 | 193908077 |
| chr1 | 193907942 | 193907992 |
| chr1 | 194154572 | 194154622 |
| chr1 | 213382532 | 213382582 |
| chr1 | 226331919 | 226331969 |
| chr1 | 229993556 | 229993606 |
| chr10 | 131670828 | 131670878 |
| chr1 | 236247591 | 236247641 |
| chr20 | 42803404 | 42803454 |
| chr20 | 52512114 | 52512164 |
| chr20 | 61302668 | 61302718 |
| chr21 | 15669373 | 15669423 |
| chr21 | 28219920 | 28219970 |
| chr21 | 29415458 | 29415508 |
| chr21 | 30422188 | 30422238 |
| chrX | 4094035 | 4094085 |
| chrX | 4931722 | 4931772 |
| chr11 | 4554884 | 4554934 |
| chrX | 18212796 | 18212846 |
| chrX | 25642827 | 25642877 |
| chrX | 39695790 | 39695840 |
| chrX | 96318456 | 96318506 |
| chr2 | 4687440 | 4687490 |
| chr2 | 21299682 | 21299732 |
| chr2 | 61021684 | 61021734 |
| chr11 | 25501812 | 25501862 |
| chr2 | 81380224 | 81380274 |
| chr2 | 107957674 | 107957724 |
| chr11 | 32871659 | 32871709 |
| chr2 | 140715701 | 140715751 |
| chr11 | 34861570 | 34861620 |
| chr2 | 163997619 | 163997669 |
| chr2 | 174374565 | 174374615 |
| chr2 | 174376460 | 174376510 |
| chr2 | 190016522 | 190016572 |
| chr2 | 205745752 | 205745802 |
| chr11 | 42859152 | 42859202 |
| chr3 | 2803508 | 2803558 |
| chr11 | 56545054 | 56545104 |
| chr3 | 96280846 | 96280896 |
| chr3 | 113173478 | 113173528 |
| chr3 | 113173689 | 113173739 |
| chr3 | 118170736 | 118170786 |
| chr3 | 150326478 | 150326528 |
| chr3 | 176452367 | 176452417 |
| chr4 | 45739600 | 45739650 |
| chr4 | 47647420 | 47647470 |
| chr4 | 59086046 | 59086096 |
| chr4 | 71166923 | 71166973 |
| chr4 | 126307615 | 126307665 |
| chr4 | 126902812 | 126902862 |
| chr4 | 134759921 | 134759971 |
| chr4 | 188667400 | 188667450 |
| chr4 | 189801568 | 189801618 |
| chr4 | 189813895 | 189813945 |
| chr4 | 189813948 | 189813998 |
| chr4 | 190023732 | 190023782 |
| chr5 | 15438485 | 15438535 |
| chr5 | 15523341 | 15523391 |
| chr5 | 42871507 | 42871557 |
| chr5 | 42871448 | 42871498 |
| chr5 | 51210887 | 51210937 |
| chr11 | 70749905 | 70749955 |
| chr5 | 125547096 | 125547146 |
| chr5 | 127378416 | 127378466 |
| chr5 | 132367138 | 132367188 |
| chr5 | 165946248 | 165946298 |
| chr6 | 47391933 | 47391983 |
| chr6 | 77337398 | 77337448 |
| chr6 | 77515790 | 77515840 |
| chr6 | 87886864 | 87886914 |
| chr6 | 121255077 | 121255127 |
| chr6 | 129044830 | 129044880 |
| chr11 | 101437929 | 101437979 |
| chr6 | 159446387 | 159446437 |
| chr7 | 4679403 | 4679453 |
| chr7 | 21748943 | 21748993 |
| chr7 | 34091613 | 34091663 |
| chr11 | 112729119 | 112729169 |
| chr7 | 96567310 | 96567360 |
| chr7 | 124012683 | 124012733 |
| chr7 | 144976415 | 144976465 |
| chr8 | 80322557 | 80322607 |
| chr9 | 2278127 | 2278177 |
| chr9 | 15278037 | 15278087 |
| chr9 | 30722552 | 30722602 |
| chr12 | 23042591 | 23042641 |
| chr12 | 70826946 | 70826996 |
| chr12 | 94957684 | 94957734 |
| chr10 | 36985494 | 36985544 |
| chr13 | 59898916 | 59898966 |
| chr13 | 62835634 | 62835684 |
| chr13 | 64632633 | 64632683 |
| chr13 | 75524840 | 75524890 |
| chr13 | 87068336 | 87068386 |
| chr14 | 39184578 | 39184628 |
| chr14 | 40975691 | 40975741 |
| chr14 | 63830219 | 63830269 |
| chr14 | 94132535 | 94132585 |
| chr14 | 94132354 | 94132404 |
| chr15 | 95180603 | 95180653 |
| chr17 | 37547462 | 37547512 |

Table 15AQ. Repeat Category LTR2

| | | |
|---|---|---|
| chr17 | 39028619 | 39028669 |
| chr17 | 39478863 | 39478913 |
| chr17 | 39946227 | 39946277 |
| chr17 | 41685175 | 41685225 |
| chr17 | 41692469 | 41692519 |
| chr17 | 47390019 | 47390069 |
| chr17 | 47630768 | 47630818 |
| chr10 | 83965962 | 83966012 |
| chr17 | 48971947 | 48971997 |
| chr10 | 84388160 | 84388210 |
| chr17 | 51703256 | 51703306 |
| chr17 | 53483866 | 53483916 |
| chr10 | 85533341 | 85533391 |
| chr10 | 85596390 | 85596440 |
| chr10 | 85596099 | 85596149 |
| chr17 | 57412405 | 57412455 |
| chr17 | 61985429 | 61985479 |
| chr17 | 65851151 | 65851201 |
| chr17 | 67240401 | 67240451 |
| chr17 | 69614713 | 69614763 |
| chr10 | 88398356 | 88398406 |
| chr17 | 75355285 | 75355335 |
| chr17 | 75597374 | 75597424 |
| chr17 | 76114760 | 76114810 |
| chr17 | 79071119 | 79071169 |
| chr17 | 79087451 | 79087501 |
| chr17 | 79371613 | 79371663 |
| chr17 | 79371457 | 79371507 |
| chr17 | 80932387 | 80932437 |
| chr17 | 81618059 | 81618109 |
| chr10 | 94076853 | 94076903 |
| chr18 | 10593595 | 10593645 |
| chr18 | 10596801 | 10596851 |
| chr18 | 11283367 | 11283417 |
| chr18 | 11935591 | 11935641 |
| chr18 | 12218754 | 12218804 |
| chr10 | 94552386 | 94552436 |
| chr18 | 13666878 | 13666928 |
| chr18 | 14771469 | 14771519 |
| chr18 | 18870150 | 18870200 |
| chr18 | 20350600 | 20350650 |
| chr18 | 23350636 | 23350686 |
| chr18 | 25255495 | 25255545 |
| chr18 | 26194652 | 26194702 |
| chr18 | 27788984 | 27789034 |
| chr18 | 31364486 | 31364536 |
| chr18 | 36714601 | 36714651 |
| chr18 | 41989894 | 41989944 |
| chr18 | 45275578 | 45275628 |
| chr18 | 45582197 | 45582247 |
| chr18 | 48526349 | 48526399 |
| chr18 | 49404836 | 49404886 |
| chr18 | 51774084 | 51774134 |
| chr18 | 53990546 | 53990596 |
| chr18 | 57081932 | 57081982 |
| chr18 | 57434154 | 57434204 |
| chr18 | 61023297 | 61023347 |
| chr18 | 67288389 | 67288439 |
| chr18 | 70350021 | 70350071 |
| chr18 | 70350428 | 70350478 |
| chr10 | 98979740 | 98979790 |
| chr10 | 98985580 | 98985630 |
| chr10 | 98986394 | 98986444 |
| chr19 | 1394000 | 1394050 |
| chr19 | 2920479 | 2920529 |
| chr19 | 5373418 | 5373468 |
| chr19 | 6140402 | 6140452 |
| chr10 | 101801489 | 101801539 |
| chr19 | 6548612 | 6548662 |
| chr10 | 5560121 | 5560171 |
| chr19 | 9679912 | 9679962 |
| chr19 | 11565111 | 11565161 |
| chr19 | 11681755 | 11681805 |
| chr19 | 11709629 | 11709679 |
| chr19 | 11710173 | 11710223 |
| chr19 | 11739028 | 11739078 |
| chr19 | 11739091 | 11739141 |
| chr19 | 11770833 | 11770883 |
| chr19 | 11771260 | 11771310 |
| chr19 | 11900230 | 11900280 |
| chr19 | 12032938 | 12032988 |
| chr19 | 12266223 | 12266273 |
| chr19 | 12336320 | 12336370 |
| chr19 | 12336879 | 12336929 |
| chr19 | 12372005 | 12372055 |
| chr19 | 12372805 | 12372855 |
| chr19 | 12412582 | 12412632 |
| chr19 | 13628057 | 13628107 |
| chr19 | 13653674 | 13653724 |
| chr19 | 17369327 | 17369377 |
| chr19 | 18357211 | 18357261 |
| chr19 | 19640096 | 19640146 |
| chr19 | 19757388 | 19757438 |
| chr19 | 21376987 | 21377037 |
| chr19 | 32928882 | 32928932 |
| chr19 | 38822936 | 38822986 |
| chr19 | 40158577 | 40158627 |
| chr19 | 40565690 | 40565740 |
| chr19 | 41110320 | 41110370 |
| chr19 | 41955259 | 41955309 |
| chr10 | 105303232 | 105303282 |
| chr19 | 46565057 | 46565107 |
| chr19 | 46714435 | 46714485 |
| chr19 | 46715236 | 46715286 |
| chr19 | 47892385 | 47892435 |
| chr19 | 47892649 | 47892699 |
| chr19 | 48609622 | 48609672 |
| chr19 | 50053270 | 50053320 |
| chr19 | 50064066 | 50064116 |
| chr19 | 50064209 | 50064259 |
| chr19 | 50078294 | 50078344 |
| chr19 | 50109581 | 50109631 |
| chr19 | 50585399 | 50585449 |
| chr19 | 50585635 | 50585685 |
| chr19 | 50585965 | 50586015 |
| chr19 | 51610271 | 51610321 |
| chr19 | 52780334 | 52780384 |
| chr19 | 52780993 | 52781043 |
| chr19 | 53727515 | 53727565 |
| chr19 | 55541854 | 55541904 |
| chr19 | 57719926 | 57719976 |
| chr19 | 57763097 | 57763147 |
| chr19 | 57854025 | 57854075 |
| chr19 | 57930997 | 57931047 |
| chr19 | 57933409 | 57933459 |
| chr19 | 58054452 | 58054502 |
| chr19 | 58121449 | 58121499 |
| chr19 | 58139364 | 58139414 |
| chr19 | 58152373 | 58152423 |
| chr19 | 58587523 | 58587573 |
| chr19 | 59483914 | 59483964 |
| chr19 | 60096805 | 60096855 |
| chr19 | 60154816 | 60154866 |
| chr19 | 60156862 | 60156912 |
| chr19 | 60162663 | 60162713 |
| chr19 | 62390683 | 62390733 |
| chr19 | 62640591 | 62640641 |
| chr19 | 63018268 | 63018318 |
| chr19 | 63018568 | 63018618 |
| chr1 | 6330271 | 6330321 |
| chr1 | 9781260 | 9781310 |
| chr10 | 117952906 | 117952956 |
| chr1 | 11671821 | 11671871 |
| chr1 | 12396086 | 12396136 |
| chr1 | 15491504 | 15491554 |
| chr10 | 118884419 | 118884469 |
| chr1 | 19950302 | 19950352 |
| chr1 | 20330029 | 20330079 |
| chr1 | 21181175 | 21181225 |
| chr1 | 28539266 | 28539316 |
| chr1 | 28795544 | 28795594 |
| chr1 | 31119950 | 31120000 |
| chr1 | 31504573 | 31504623 |
| chr1 | 35040144 | 35040194 |
| chr1 | 35042932 | 35042982 |
| chr1 | 36230083 | 36230133 |
| chr1 | 37449091 | 37449141 |
| chr10 | 121396854 | 121396904 |
| chr1 | 42494554 | 42494604 |
| chr1 | 42499910 | 42499960 |
| chr10 | 122418270 | 122418320 |
| chr1 | 46160842 | 46160892 |
| chr1 | 46167580 | 46167630 |
| chr1 | 48031919 | 48031969 |
| chr1 | 61923197 | 61923247 |
| chr1 | 68747431 | 68747481 |
| chr1 | 70352948 | 70352998 |
| chr1 | 84938478 | 84938528 |
| chr1 | 89115507 | 89115557 |
| chr1 | 91599728 | 91599778 |
| chr1 | 91607444 | 91607494 |
| chr1 | 94635003 | 94635053 |
| chr1 | 101499633 | 101499683 |
| chr1 | 108864439 | 108864489 |
| chr1 | 109294144 | 109294194 |
| chr1 | 110171815 | 110171865 |
| chr1 | 112329707 | 112329757 |
| chr1 | 112711044 | 112711094 |
| chr1 | 117104818 | 117104868 |
| chr1 | 143138973 | 143139023 |
| chr1 | 144204100 | 144204150 |
| chr1 | 144204586 | 144204636 |
| chr1 | 145495128 | 145495178 |
| chr10 | 127063354 | 127063404 |
| chr1 | 151560957 | 151561007 |
| chr1 | 151651081 | 151651131 |
| chr1 | 152826889 | 152826939 |
| chr1 | 153370515 | 153370565 |
| chr1 | 153488208 | 153488258 |
| chr10 | 127836298 | 127836348 |
| chr1 | 154460528 | 154460578 |
| chr1 | 158583796 | 158583846 |
| chr1 | 158590629 | 158590679 |
| chr10 | 128376080 | 128376130 |
| chr1 | 164876979 | 164877029 |
| chr1 | 165026379 | 165026429 |
| chr1 | 166209285 | 166209335 |
| chr1 | 168460105 | 168460155 |
| chr1 | 168582047 | 168582097 |
| chr1 | 180799392 | 180799442 |
| chr1 | 180799206 | 180799256 |
| chr1 | 190494975 | 190495025 |
| chr1 | 190653142 | 190653192 |
| chr1 | 199425162 | 199425212 |
| chr1 | 200686949 | 200686999 |
| chr1 | 201162718 | 201162768 |
| chr1 | 202285654 | 202285704 |
| chr1 | 204672681 | 204672731 |
| chr1 | 206360577 | 206360627 |
| chr1 | 209445107 | 209445157 |
| chr1 | 217611296 | 217611346 |
| chr1 | 217947911 | 217947961 |
| chr1 | 221770585 | 221770635 |
| chr1 | 222624864 | 222624914 |
| chr1 | 223012954 | 223013004 |
| chr1 | 223609677 | 223609727 |
| chr1 | 226787766 | 226787816 |
| chr1 | 229134333 | 229134383 |
| chr1 | 229134392 | 229134442 |
| chr1 | 235949708 | 235949758 |
| chr1 | 236387703 | 236387753 |
| chr1 | 237134782 | 237134832 |
| chr1 | 238312275 | 238312325 |
| chr1 | 240031834 | 240031884 |
| chr1 | 242235032 | 242235082 |
| chr1 | 242234817 | 242234867 |
| chr1 | 243700264 | 243700314 |
| chr1 | 243856550 | 243856600 |
| chr1 | 243864247 | 243864297 |
| chr1 | 244480603 | 244480653 |
| chr1 | 244959264 | 244959314 |
| chr1 | 244966320 | 244966370 |
| chr1 | 245039049 | 245039099 |
| chr1 | 245105374 | 245105424 |
| chr1 | 245756160 | 245756210 |
| chr20 | 8818304 | 8818354 |
| chr20 | 15783857 | 15783907 |
| chr20 | 15961266 | 15961316 |
| chr20 | 16594607 | 16594657 |
| chr20 | 16782255 | 16782305 |
| chr20 | 24895512 | 24895562 |
| chr20 | 24929333 | 24929383 |
| chr20 | 25629099 | 25629149 |
| chr20 | 33635124 | 33635174 |
| chr20 | 34451466 | 34451516 |
| chr20 | 34451828 | 34451878 |
| chr20 | 35916384 | 35916434 |
| chr20 | 42617721 | 42617771 |
| chr20 | 58819322 | 58819372 |
| chr20 | 62587734 | 62587784 |
| chr20 | 63283441 | 63283491 |
| chr21 | 13549685 | 13549735 |
| chr21 | 16932929 | 16932979 |
| chr21 | 20293673 | 20293723 |
| chr21 | 22492031 | 22492081 |
| chr21 | 22856440 | 22856490 |
| chr21 | 24036376 | 24036426 |
| chr21 | 28243281 | 28243331 |
| chr21 | 30326358 | 30326408 |
| chr21 | 30529734 | 30529784 |
| chr21 | 31317559 | 31317609 |
| chr21 | 32952678 | 32952728 |
| chr21 | 33910458 | 33910508 |
| chr21 | 38465673 | 38465723 |
| chr21 | 39427282 | 39427332 |
| chr11 | 799708 | 799758 |
| chr21 | 43241599 | 43241649 |
| chr21 | 43273409 | 43273459 |
| chr21 | 43422050 | 43422100 |
| chr21 | 43432644 | 43432694 |
| chr21 | 43439482 | 43439532 |
| chr21 | 43447498 | 43447548 |
| chr21 | 44054787 | 44054837 |
| chr21 | 45152007 | 45152057 |
| chr21 | 46167919 | 46167969 |
| chr21 | 46246189 | 46246239 |
| chr21 | 46870421 | 46870471 |
| chr22 | 16995482 | 16995532 |
| chr22 | 17867323 | 17867373 |
| chr22 | 19716208 | 19716258 |
| chr22 | 28136508 | 28136558 |
| chr22 | 29074434 | 29074484 |
| chr10 | 9425346 | 9425396 |
| chr22 | 34084277 | 34084327 |
| chr22 | 34918325 | 34918375 |
| chr22 | 35615523 | 35615573 |
| chr22 | 36231410 | 36231460 |
| chr22 | 39387177 | 39387227 |
| chr22 | 39392658 | 39392708 |
| chr22 | 40399586 | 40399636 |
| chr22 | 41366555 | 41366605 |
| chr22 | 42612276 | 42612326 |
| chr22 | 43421174 | 43421224 |
| chr22 | 44113957 | 44114007 |
| chr11 | 3007894 | 3007944 |
| chr11 | 3338103 | 3338153 |
| chr11 | 3458888 | 3458938 |
| chr22 | 49104351 | 49104401 |
| chr11 | 3629871 | 3629921 |
| chr22 | 49211354 | 49211404 |
| chr11 | 3629991 | 3630041 |
| chr11 | 3630577 | 3630627 |
| chrX | 4067913 | 4067963 |
| chrX | 4627084 | 4627134 |
| chrX | 5687488 | 5687538 |
| chrX | 9964303 | 9964353 |
| chrX | 9969428 | 9969478 |
| chrX | 18244648 | 18244698 |
| chrX | 19135136 | 19135186 |
| chrX | 22874287 | 22874337 |
| chrX | 23078841 | 23078891 |
| chrX | 23079764 | 23079814 |
| chr11 | 5676681 | 5676731 |
| chrX | 34878077 | 34878127 |
| chrX | 36849294 | 36849344 |
| chrX | 39741848 | 39741898 |
| chrX | 42073388 | 42073438 |
| chrX | 43679421 | 43679471 |
| chrX | 52464224 | 52464274 |
| chrX | 52502437 | 52502487 |
| chrX | 55024806 | 55024856 |
| chrX | 61503162 | 61503212 |
| chrX | 63282890 | 63282940 |
| chrX | 72004917 | 72004967 |
| chrX | 72336913 | 72336963 |
| chrX | 72429315 | 72429365 |
| chrX | 81671718 | 81671768 |
| chrX | 89305745 | 89305795 |
| chrX | 90838663 | 90838713 |
| chrX | 91034983 | 91035033 |
| chrX | 91456533 | 91456583 |
| chrX | 91517518 | 91517568 |
| chrX | 91898017 | 91898067 |
| chrX | 91902038 | 91902088 |
| chrX | 92731936 | 92731986 |
| chrX | 93687606 | 93687656 |
| chrX | 96316297 | 96316347 |
| chrX | 101210726 | 101210776 |
| chrX | 101885783 | 101885833 |
| chrX | 101892345 | 101892395 |
| chrX | 104123657 | 104123707 |
| chr11 | 9905057 | 9905107 |
| chrX | 111818994 | 111819044 |
| chrX | 111993667 | 111993717 |
| chrX | 112065813 | 112065863 |
| chrX | 113536277 | 113536327 |
| chrX | 113543646 | 113543696 |
| chrX | 117670386 | 117670436 |
| chrX | 117780088 | 117780138 |
| chrX | 118401778 | 118401828 |
| chrX | 118916368 | 118916418 |
| chrX | 125013315 | 125013365 |
| chrX | 125757667 | 125757717 |
| chrX | 140047110 | 140047160 |
| chrX | 140768038 | 140768088 |
| chrX | 144505686 | 144505736 |
| chrX | 145018136 | 145018186 |
| chrX | 145020894 | 145020944 |
| chrX | 147342261 | 147342311 |
| chrX | 147470265 | 147470315 |
| chrY | 3932711 | 3932761 |
| chrY | 5695388 | 5695438 |
| chrY | 12921017 | 12921067 |
| chrY | 13412083 | 13412133 |
| chrY | 13821906 | 13821956 |
| chrY | 13821731 | 13821781 |
| chrY | 14403477 | 14403527 |
| chrY | 17871676 | 17871726 |
| chrY | 20410730 | 20410780 |
| chrY | 21567580 | 21567630 |
| chr2 | 588312 | 588362 |
| chr2 | 588190 | 588240 |
| chr2 | 5002838 | 5002888 |
| chr2 | 7747326 | 7747376 |
| chr2 | 9960860 | 9960910 |
| chr2 | 13446752 | 13446802 |
| chr2 | 13687966 | 13688016 |
| chr2 | 13695140 | 13695190 |
| chr2 | 13695235 | 13695285 |
| chr2 | 14958986 | 14959036 |
| chr2 | 21007473 | 21007523 |
| chr2 | 21008189 | 21008239 |
| chr2 | 25909581 | 25909631 |
| chr11 | 18613976 | 18614026 |
| chr11 | 18621921 | 18621971 |
| chr2 | 34056458 | 34056508 |
| chr2 | 38871144 | 38871194 |
| chr2 | 40601916 | 40601966 |
| chr2 | 41241795 | 41241845 |
| chr2 | 42831146 | 42831196 |
| chr2 | 48407905 | 48407955 |
| chr2 | 51288343 | 51288393 |
| chr2 | 52639544 | 52639594 |
| chr2 | 55473500 | 55473550 |
| chr2 | 57223550 | 57223600 |
| chr2 | 62126818 | 62126868 |
| chr2 | 65089272 | 65089322 |
| chr2 | 65101094 | 65101144 |
| chr11 | 24503029 | 24503079 |
| chr2 | 71248028 | 71248078 |
| chr2 | 71590282 | 71590332 |
| chr2 | 75661610 | 75661660 |
| chr2 | 77862406 | 77862456 |
| chr2 | 86758307 | 86758357 |
| chr2 | 95701938 | 95701988 |
| chr2 | 97069591 | 97069641 |
| chr2 | 98272831 | 98272881 |
| chr2 | 100608776 | 100608826 |
| chr2 | 106217414 | 106217464 |
| chr2 | 106716520 | 106716570 |
| chr2 | 106765290 | 106765340 |
| chr2 | 108761618 | 108761668 |
| chr2 | 108769207 | 108769257 |
| chr2 | 109356730 | 109356780 |
| chr2 | 111532769 | 111532819 |
| chr2 | 113703067 | 113703117 |
| chr2 | 117982939 | 117982989 |
| chr2 | 117988174 | 117988224 |
| chr2 | 122539220 | 122539270 |
| chr2 | 123245158 | 123245208 |
| chr2 | 128022307 | 128022357 |
| chr2 | 128365154 | 128365204 |
| chr2 | 128534227 | 128534277 |
| chr2 | 128534793 | 128534843 |
| chr2 | 128832618 | 128832668 |
| chr2 | 129534141 | 129534191 |
| chr2 | 132062687 | 132062737 |
| chr2 | 132442114 | 132442164 |
| chr2 | 133526207 | 133526257 |
| chr2 | 136112166 | 136112216 |
| chr2 | 139505991 | 139506041 |
| chr2 | 142562144 | 142562194 |
| chr2 | 157477584 | 157477634 |
| chr2 | 159940655 | 159940705 |
| chr2 | 167668621 | 167668671 |
| chr2 | 167826266 | 167826316 |
| chr2 | 171272273 | 171272323 |
| chr2 | 171973724 | 171973774 |
| chr2 | 173604336 | 173604386 |
| chr2 | 174376460 | 174376510 |
| chr2 | 177087324 | 177087374 |
| chr2 | 182557052 | 182557102 |
| chr2 | 187308758 | 187308808 |
| chr2 | 188583849 | 188583899 |
| chr2 | 188633405 | 188633455 |
| chr11 | 39638352 | 39638402 |
| chr2 | 200943737 | 200943787 |
| chr2 | 206421512 | 206421562 |
| chr2 | 212397308 | 212397358 |
| chr2 | 215903950 | 215904000 |
| chr2 | 224271102 | 224271152 |
| chr2 | 228789477 | 228789527 |
| chr2 | 228872995 | 228873045 |
| chr2 | 231741744 | 231741794 |
| chr2 | 232168171 | 232168221 |
| chr2 | 232468472 | 232468522 |
| chr2 | 232476101 | 232476151 |
| chr2 | 235053053 | 235053103 |
| chr2 | 235116680 | 235116730 |
| chr2 | 238473878 | 238473928 |
| chr3 | 3218941 | 3218991 |
| chr3 | 3663713 | 3663763 |
| chr3 | 5239468 | 5239518 |
| chr3 | 9607356 | 9607406 |
| chr3 | 10061654 | 10061704 |
| chr3 | 15405995 | 15406045 |
| chr3 | 19498405 | 19498455 |
| chr3 | 20387292 | 20387342 |
| chr3 | 32093173 | 32093223 |
| chr11 | 47633579 | 47633629 |
| chr11 | 47755537 | 47755587 |
| chr3 | 36677325 | 36677375 |
| chr3 | 39920284 | 39920334 |
| chr3 | 43680816 | 43680866 |
| chr11 | 48987867 | 48987917 |
| chr3 | 47182135 | 47182185 |
| chr11 | 54965097 | 54965147 |
| chr3 | 52278595 | 52278645 |
| chr3 | 53339339 | 53339389 |
| chr3 | 54166345 | 54166395 |
| chr3 | 63916588 | 63916638 |
| chr3 | 70830143 | 70830193 |
| chr3 | 76581063 | 76581113 |
| chr3 | 82429291 | 82429341 |
| chr3 | 99178056 | 99178106 |
| chr3 | 102652658 | 102652708 |
| chr3 | 105584413 | 105584463 |
| chr3 | 110264129 | 110264179 |
| chr3 | 110433040 | 110433090 |
| chr3 | 111684064 | 111684114 |
| chr3 | 115143053 | 115143103 |
| chr3 | 120295590 | 120295640 |
| chr3 | 120741078 | 120741128 |
| chr3 | 121351668 | 121351718 |
| chr3 | 122958720 | 122958770 |
| chr11 | 60636237 | 60636287 |
| chr3 | 125770962 | 125771012 |
| chr3 | 127106844 | 127106894 |
| chr3 | 133342776 | 133342826 |
| chr3 | 133350175 | 133350225 |
| chr3 | 135060147 | 135060197 |
| chr3 | 139463817 | 139463867 |
| chr3 | 145215577 | 145215627 |
| chr3 | 147051172 | 147051222 |
| chr3 | 149564090 | 149564140 |
| chr3 | 151637389 | 151637439 |
| chr3 | 153752600 | 153752650 |
| chr3 | 155873727 | 155873777 |
| chr3 | 158626196 | 158626246 |
| chr3 | 160221378 | 160221428 |
| chr3 | 162982713 | 162982763 |
| chr3 | 163748181 | 163748231 |
| chr3 | 164092710 | 164092760 |
| chr3 | 171116443 | 171116493 |
| chr3 | 174310222 | 174310272 |
| chr3 | 176659137 | 176659187 |
| chr3 | 183829667 | 183829717 |
| chr3 | 187972204 | 187972254 |
| chr3 | 187972142 | 187972192 |
| chr3 | 196986684 | 196986734 |
| chr3 | 197110571 | 197110621 |
| chr4 | 2011788 | 2011838 |
| chr4 | 2021515 | 2021565 |
| chr10 | 13196866 | 13196916 |
| chr4 | 3661548 | 3661598 |
| chr4 | 3662498 | 3662548 |
| chr4 | 4414658 | 4414708 |
| chr4 | 5678869 | 5678919 |
| chr4 | 8288791 | 8288841 |
| chr4 | 8628791 | 8628841 |
| chr4 | 8710528 | 8710578 |
| chr4 | 13180233 | 13180283 |
| chr4 | 15243603 | 15243653 |
| chr4 | 17320151 | 17320201 |
| chr4 | 18440654 | 18440704 |
| chr4 | 18945698 | 18945748 |
| chr4 | 22351979 | 22352029 |
| chr4 | 22357975 | 22358025 |
| chr4 | 24835719 | 24835769 |
| chr4 | 25358912 | 25358962 |
| chr4 | 28033977 | 28034027 |
| chr4 | 28039035 | 28039085 |
| chr4 | 29979336 | 29979386 |
| chr4 | 33128852 | 33128902 |
| chr4 | 36805197 | 36805247 |
| chr4 | 37498058 | 37498108 |
| chr4 | 37550764 | 37550814 |
| chr11 | 64803238 | 64803288 |
| chr4 | 39899227 | 39899277 |
| chr4 | 45138564 | 45138614 |
| chr4 | 47832360 | 47832410 |
| chr4 | 53483617 | 53483667 |
| chr4 | 53495607 | 53495657 |
| chr4 | 57190198 | 57190248 |
| chr4 | 60049441 | 60049491 |
| chr4 | 61124947 | 61124997 |
| chr4 | 65927420 | 65927470 |
| chr4 | 70232770 | 70232820 |
| chr4 | 70232602 | 70232652 |
| chr4 | 70963685 | 70963735 |
| chr4 | 75666261 | 75666311 |
| chr4 | 79227806 | 79227856 |
| chr4 | 84574672 | 84574722 |
| chr4 | 92738984 | 92739034 |
| chr4 | 93425519 | 93425569 |
| chr4 | 100403699 | 100403749 |
| chr4 | 101492283 | 101492333 |
| chr4 | 110697888 | 110697938 |
| chr4 | 124065534 | 124065584 |
| chr4 | 124816217 | 124816267 |
| chr4 | 125322358 | 125322408 |
| chr4 | 126926330 | 126926380 |
| chr4 | 130070888 | 130070938 |
| chr4 | 130084180 | 130084230 |
| chr4 | 135855090 | 135855140 |
| chr4 | 135926320 | 135926370 |
| chr4 | 148025224 | 148025274 |
| chr11 | 67108350 | 67108400 |
| chr11 | 67109507 | 67109557 |
| chr11 | 67111258 | 67111308 |
| chr11 | 67112798 | 67112848 |
| chr4 | 159727410 | 159727460 |
| chr11 | 67133681 | 67133731 |
| chr11 | 67145556 | 67145606 |
| chr4 | 164392232 | 164392282 |
| chr4 | 164395794 | 164395844 |
| chr4 | 169513106 | 169513156 |
| chr11 | 67552406 | 67552456 |
| chr4 | 171526735 | 171526785 |
| chr4 | 172040296 | 172040346 |
| chr4 | 173128612 | 173128662 |
| chr4 | 173194836 | 173194886 |
| chr4 | 174032461 | 174032511 |
| chr4 | 174343047 | 174343097 |
| chr4 | 174716255 | 174716305 |
| chr4 | 174723263 | 174723313 |
| chr4 | 177807252 | 177807302 |
| chr4 | 178997835 | 178997885 |
| chr4 | 179019694 | 179019744 |
| chr4 | 180429805 | 180429855 |
| chr4 | 184472398 | 184472448 |
| chr4 | 184994715 | 184994765 |
| chr4 | 185045332 | 185045382 |
| chr4 | 186127705 | 186127755 |
| chr4 | 186238592 | 186238642 |
| chr4 | 188077113 | 188077163 |
| chr11 | 68427881 | 68427931 |
| chr4 | 191335863 | 191335913 |
| chr5 | 227094 | 227144 |
| chr5 | 1613359 | 1613409 |
| chr5 | 7095436 | 7095486 |
| chr5 | 7141189 | 7141239 |
| chr5 | 18845879 | 18845929 |
| chr5 | 26892790 | 26892840 |
| chr5 | 27166782 | 27166832 |
| chr5 | 29442113 | 29442163 |
| chr5 | 31645228 | 31645278 |
| chr5 | 36158968 | 36159018 |
| chr11 | 69962547 | 69962597 |
| chr5 | 39542540 | 39542590 |
| chr5 | 39711015 | 39711065 |
| chr5 | 42859327 | 42859377 |
| chr5 | 43125518 | 43125568 |
| chr5 | 52860959 | 52861009 |
| chr5 | 55659135 | 55659185 |
| chr5 | 58474512 | 58474562 |
| chr5 | 59941893 | 59941943 |
| chr5 | 64486100 | 64486150 |
| chr5 | 68607874 | 68607924 |
| chr5 | 74054181 | 74054231 |
| chr5 | 74057147 | 74057197 |
| chr5 | 78595338 | 78595388 |
| chr5 | 79752149 | 79752199 |
| chr5 | 81653083 | 81653133 |
| chr5 | 81797942 | 81797992 |
| chr5 | 85840494 | 85840544 |
| chr5 | 85950481 | 85950531 |
| chr5 | 104206301 | 104206351 |
| chr11 | 71862318 | 71862368 |
| chr5 | 116187803 | 116187853 |
| chr5 | 116448700 | 116448750 |
| chr11 | 72002084 | 72002134 |
| chr5 | 138643314 | 138643364 |
| chr5 | 138651247 | 138651297 |
| chr5 | 141644499 | 141644549 |
| chr11 | 73390859 | 73390909 |
| chr11 | 73968586 | 73968636 |
| chr11 | 73971842 | 73971892 |
| chr5 | 157027162 | 157027212 |
| chr5 | 158774642 | 158774692 |
| chr5 | 162727134 | 162727184 |
| chr5 | 164625597 | 164625647 |
| chr5 | 165536408 | 165536458 |
| chr5 | 167886974 | 167887024 |
| chr5 | 169858482 | 169858532 |
| chr5 | 171332618 | 171332668 |
| chr5 | 177644195 | 177644245 |
| chr5 | 177816809 | 177816859 |
| chr5 | 178319823 | 178319873 |
| chr5 | 178596404 | 178596454 |
| chr5 | 180057778 | 180057828 |
| chr5 | 180318862 | 180318912 |
| chr5 | 180404795 | 180404845 |
| chr5 | 180518630 | 180518680 |
| chr6 | 3800847 | 3800897 |
| chr6 | 10799433 | 10799483 |
| chr6 | 12634531 | 12634581 |
| chr6 | 15084582 | 15084632 |
| chr6 | 15677537 | 15677587 |
| chr6 | 17435075 | 17435125 |
| chr11 | 80092298 | 80092348 |
| chr6 | 21574229 | 21574279 |
| chr11 | 80470448 | 80470498 |
| chr11 | 81389164 | 81389214 |
| chr6 | 26074279 | 26074329 |
| chr11 | 81394145 | 81394195 |
| chr6 | 26183592 | 26183642 |
| chr6 | 26670219 | 26670269 |
| chr6 | 27960286 | 27960336 |
| chr6 | 28411705 | 28411755 |
| chr6 | 29768715 | 29768765 |
| chr11 | 82784357 | 82784407 |
| chr6 | 31678079 | 31678129 |
| chr6 | 31924180 | 31924230 |
| chr6 | 31925025 | 31925075 |
| chr11 | 85368382 | 85368432 |
| chr6 | 38811313 | 38811363 |
| chr6 | 42788729 | 42788779 |
| chr6 | 43657957 | 43658007 |
| chr6 | 47649941 | 47649991 |
| chr6 | 56986133 | 56986183 |
| chr6 | 63555999 | 63556049 |
| chr6 | 65010255 | 65010305 |
| chr6 | 66334642 | 66334692 |
| chr6 | 66643839 | 66643889 |
| chr6 | 68885928 | 68885978 |
| chr6 | 69251312 | 69251362 |
| chr11 | 90197308 | 90197358 |
| chr6 | 75174105 | 75174155 |
| chr6 | 75179909 | 75179959 |
| chr6 | 89368118 | 89368168 |
| chr6 | 93552569 | 93552619 |
| chr10 | 912747 | 912797 |
| chr6 | 101445672 | 101445722 |
| chr6 | 107116179 | 107116229 |
| chr6 | 108640785 | 108640835 |
| chr6 | 111398568 | 111398618 |
| chr6 | 111530203 | 111530253 |
| chr10 | 914729 | 914779 |
| chr6 | 116688533 | 116688583 |
| chr11 | 95355336 | 95355386 |
| chr6 | 121706665 | 121706715 |
| chr6 | 123057462 | 123057512 |
| chr6 | 131287208 | 131287258 |
| chr6 | 131688049 | 131688099 |
| chr6 | 139591211 | 139591261 |
| chr6 | 141772157 | 141772207 |
| chr6 | 147754972 | 147755022 |
| chr6 | 153802926 | 153802976 |
| chr6 | 161778185 | 161778235 |
| chr6 | 164343114 | 164343164 |
| chr6 | 166878666 | 166878716 |
| chr6 | 167281024 | 167281074 |
| chr6 | 169812278 | 169812328 |
| chr6 | 169813003 | 169813053 |
| chr7 | 581860 | 581910 |
| chr7 | 1111718 | 1111768 |
| chr11 | 107559488 | 107559538 |
| chr7 | 1914999 | 1915049 |
| chr11 | 107813092 | 107813142 |
| chr7 | 4805043 | 4805093 |
| chr7 | 6734197 | 6734247 |
| chr7 | 7580027 | 7580077 |
| chr7 | 12373055 | 12373105 |
| chr7 | 23584344 | 23584394 |
| chr7 | 24507279 | 24507329 |
| chr7 | 24577109 | 24577159 |
| chr7 | 30590054 | 30590104 |
| chr7 | 32544032 | 32544082 |
| chr7 | 32544934 | 32544984 |
| chr7 | 32863621 | 32863671 |
| chr7 | 36415370 | 36415420 |
| chr7 | 39513963 | 39514013 |
| chr7 | 39663241 | 39663291 |
| chr7 | 43605111 | 43605161 |
| chr7 | 43699001 | 43699051 |
| chr7 | 43701354 | 43701404 |
| chr7 | 44628855 | 44628905 |
| chr7 | 48263214 | 48263264 |
| chr7 | 51923255 | 51923305 |
| chr7 | 54689646 | 54689696 |
| chr7 | 55715185 | 55715235 |
| chr7 | 57214790 | 57214840 |
| chr7 | 57345133 | 57345183 |
| chr7 | 63382628 | 63382678 |
| chr7 | 63386794 | 63386844 |
| chr7 | 63467287 | 63467337 |
| chr7 | 63661619 | 63661669 |
| chr7 | 64189290 | 64189340 |
| chr7 | 65498298 | 65498348 |
| chr7 | 66401858 | 66401908 |
| chr7 | 67230852 | 67230902 |
| chr7 | 70313913 | 70313963 |
| chr7 | 72965786 | 72965836 |
| chr7 | 72984858 | 72984908 |
| chr7 | 75120415 | 75120465 |
| chr7 | 75262888 | 75262938 |
| chr7 | 91779609 | 91779659 |
| chr7 | 97504904 | 97504954 |
| chr7 | 100048567 | 100048617 |
| chr7 | 100679139 | 100679189 |
| chr7 | 100679018 | 100679068 |
| chr7 | 102353546 | 102353596 |
| chr7 | 104429609 | 104429659 |
| chr7 | 104716150 | 104716200 |
| chr7 | 106587234 | 106587284 |
| chr7 | 114778681 | 114778731 |
| chr7 | 125853544 | 125853594 |
| chr7 | 136369064 | 136369114 |
| chr7 | 138225573 | 138225623 |
| chr11 | 118610680 | 118610730 |
| chr7 | 144448326 | 144448376 |
| chr11 | 119552407 | 119552457 |
| chr7 | 152936797 | 152936847 |
| chr7 | 153338632 | 153338682 |
| chr7 | 154112101 | 154112151 |
| chr7 | 154111917 | 154111967 |
| chr7 | 156308736 | 156308786 |
| chr8 | 236845 | 236895 |
| chr8 | 419731 | 419781 |
| chr8 | 2943936 | 2943986 |
| chr8 | 5664060 | 5664110 |
| chr8 | 6913386 | 6913436 |
| chr8 | 9144681 | 9144731 |
| chr8 | 11806400 | 11806450 |
| chr8 | 11808195 | 11808245 |
| chr8 | 11830995 | 11831045 |
| chr8 | 12409569 | 12409619 |
| chr8 | 12580440 | 12580490 |
| chr8 | 15418429 | 15418479 |
| chr8 | 18479985 | 18480035 |
| chr8 | 24115056 | 24115106 |
| chr8 | 28475553 | 28475603 |
| chr8 | 30451685 | 30451735 |
| chr8 | 33112574 | 33112624 |
| chr8 | 33574521 | 33574571 |
| chr8 | 41471654 | 41471704 |
| chr8 | 48951898 | 48951948 |
| chr8 | 49043557 | 49043607 |
| chr8 | 56663509 | 56663559 |
| chr8 | 56738736 | 56738786 |
| chr8 | 57864910 | 57864960 |
| chr8 | 57864660 | 57864710 |
| chr8 | 59653843 | 59653893 |
| chr8 | 67221695 | 67221745 |
| chr8 | 71497749 | 71497799 |
| chr8 | 74994651 | 74994701 |
| chr8 | 78583485 | 78583535 |
| chr8 | 95246983 | 95247033 |
| chr8 | 103402296 | 103402346 |
| chr8 | 103970520 | 103970570 |
| chr8 | 108139249 | 108139299 |
| chr8 | 112794021 | 112794071 |
| chr8 | 116041190 | 116041240 |
| chr8 | 122774184 | 122774234 |
| chr8 | 124455023 | 124455073 |
| chr8 | 134577670 | 134577720 |
| chr8 | 139954482 | 139954532 |
| chr8 | 141200394 | 141200444 |
| chr8 | 144223084 | 144223134 |
| chr8 | 144525565 | 144525615 |
| chr8 | 144734822 | 144734872 |
| chr8 | 144815880 | 144815930 |
| chr8 | 144868006 | 144868056 |
| chr8 | 144870176 | 144870226 |
| chr8 | 146299504 | 146299554 |
| chr9 | 4398875 | 4398925 |
| chr9 | 5221232 | 5221282 |
| chr9 | 5707479 | 5707529 |
| chr9 | 6995119 | 6995169 |
| chr9 | 7223834 | 7223884 |
| chr9 | 15129441 | 15129491 |
| chr9 | 19188085 | 19188135 |
| chr9 | 32297420 | 32297470 |
| chr9 | 34630320 | 34630370 |
| chr9 | 37108937 | 37108987 |
| chr9 | 37452896 | 37452946 |
| chr9 | 64750083 | 64750133 |
| chr9 | 73779268 | 73779318 |
| chr9 | 74434560 | 74434610 |
| chr9 | 82414103 | 82414153 |
| chr9 | 84588809 | 84588859 |
| chr9 | 85537862 | 85537912 |
| chr9 | 86164713 | 86164763 |
| chr9 | 86165447 | 86165497 |
| chr9 | 86278149 | 86278199 |
| chr9 | 86492458 | 86492508 |
| chr9 | 89431631 | 89431681 |
| chr9 | 92764974 | 92765024 |
| chr9 | 96256903 | 96256953 |
| chr9 | 98510946 | 98510996 |
| chr9 | 101445869 | 101445919 |
| chr9 | 101904997 | 101905047 |
| chr9 | 102777072 | 102777122 |
| chr9 | 103696290 | 103696340 |
| chr12 | 557741 | 557791 |
| chr12 | 668516 | 668566 |
| chr9 | 125842875 | 125842925 |
| chr9 | 126187445 | 126187495 |
| chr9 | 131559432 | 131559482 |
| chr9 | 131560295 | 131560345 |
| chr9 | 131623764 | 131623814 |
| chr9 | 131624234 | 131624284 |
| chr9 | 132107939 | 132107989 |
| chr9 | 133768441 | 133768491 |
| chr9 | 133775083 | 133775133 |
| chr9 | 134011420 | 134011470 |
| chr9 | 135379180 | 135379230 |
| chr9 | 135845549 | 135845599 |
| chr12 | 3433959 | 3434009 |
| chr10 | 22165642 | 22165692 |
| chr12 | 7586337 | 7586387 |
| chr12 | 8117084 | 8117134 |
| chr12 | 8326207 | 8326257 |
| chr12 | 12151599 | 12151649 |
| chr12 | 17328085 | 17328135 |
| chr12 | 31794390 | 31794440 |
| chr12 | 31918372 | 31918422 |
| chr12 | 32456617 | 32456667 |
| chr12 | 39269476 | 39269526 |
| chr12 | 41122714 | 41122764 |
| chr12 | 48420535 | 48420585 |
| chr12 | 50945002 | 50945052 |
| chr12 | 55406124 | 55406174 |
| chr12 | 59258225 | 59258275 |
| chr12 | 63674690 | 63674740 |
| chr12 | 67850931 | 67850981 |
| chr12 | 71498252 | 71498302 |
| chr12 | 72944885 | 72944935 |
| chr12 | 72952953 | 72953003 |
| chr12 | 86149346 | 86149396 |
| chr12 | 87080333 | 87080383 |
| chr12 | 91836829 | 91836879 |
| chr10 | 29653393 | 29653443 |
| chr12 | 93713349 | 93713399 |
| chr12 | 94284872 | 94284922 |
| chr12 | 94457521 | 94457571 |
| chr12 | 99061198 | 99061248 |
| chr12 | 99529322 | 99529372 |
| chr12 | 106075761 | 106075811 |
| chr10 | 30769582 | 30769632 |
| chr12 | 112006296 | 112006346 |
| chr12 | 118805078 | 118805128 |
| chr12 | 123664698 | 123664748 |
| chr12 | 124945329 | 124945379 |
| chr10 | 34116294 | 34116344 |
| chr12 | 126842488 | 126842538 |
| chr10 | 35262240 | 35262290 |
| chr12 | 131255695 | 131255745 |
| chr12 | 131776885 | 131776935 |
| chr13 | 17555358 | 17555408 |
| chr13 | 18906462 | 18906512 |
| chr13 | 19129242 | 19129292 |
| chr13 | 21255392 | 21255442 |
| chr13 | 23299820 | 23299870 |
| chr13 | 23309965 | 23310015 |
| chr13 | 23760934 | 23760984 |
| chr13 | 26497864 | 26497914 |
| chr10 | 37049645 | 37049695 |
| chr13 | 29949747 | 29949797 |
| chr13 | 29973437 | 29973487 |
| chr13 | 33069039 | 33069089 |
| chr13 | 37510399 | 37510449 |
| chr13 | 39673030 | 39673080 |
| chr13 | 39828061 | 39828111 |
| chr13 | 41378934 | 41378984 |
| chr13 | 47613614 | 47613664 |
| chr13 | 50531518 | 50531568 |
| chr13 | 53978335 | 53978385 |
| chr13 | 54234495 | 54234545 |
| chr13 | 54350050 | 54350100 |
| chr13 | 60766651 | 60766701 |
| chr13 | 66141950 | 66142000 |
| chr13 | 68174999 | 68175049 |
| chr13 | 69398473 | 69398523 |
| chr13 | 79762676 | 79762726 |
| chr13 | 82088346 | 82088396 |
| chr13 | 82787309 | 82787359 |
| chr13 | 84402822 | 84402872 |
| chr13 | 86994951 | 86995001 |
| chr10 | 43085538 | 43085588 |
| chr10 | 43148067 | 43148117 |
| chr10 | 43149330 | 43149380 |
| chr10 | 43148997 | 43149047 |
| chr10 | 43149450 | 43149500 |
| chr13 | 93720916 | 93720966 |
| chr13 | 100790321 | 100790371 |
| chr13 | 101054652 | 101054702 |
| chr13 | 101054501 | 101054551 |
| chr13 | 106261452 | 106261502 |
| chr13 | 111894512 | 111894562 |
| chr13 | 111896321 | 111896371 |
| chr13 | 112916909 | 112916959 |
| chr14 | 29699751 | 29699801 |
| chr14 | 32422564 | 32422614 |
| chr14 | 38222090 | 38222140 |
| chr14 | 42384193 | 42384243 |
| chr14 | 46394305 | 46394355 |
| chr14 | 47264521 | 47264571 |
| chr14 | 48076600 | 48076650 |
| chr14 | 48076925 | 48076975 |
| chr14 | 54430030 | 54430080 |
| chr14 | 62106310 | 62106360 |
| chr14 | 63914479 | 63914529 |
| chr14 | 64413606 | 64413656 |
| chr14 | 72007549 | 72007599 |
| chr14 | 72040326 | 72040376 |
| chr14 | 78921071 | 78921121 |
| chr14 | 81799144 | 81799194 |
| chr14 | 82051375 | 82051425 |
| chr10 | 54112943 | 54112993 |
| chr14 | 88414597 | 88414647 |
| chr10 | 54741706 | 54741756 |
| chr14 | 91205145 | 91205195 |
| chr14 | 100704576 | 100704626 |
| chr10 | 59198618 | 59198668 |
| chr14 | 101710619 | 101710669 |
| chr14 | 103476977 | 103477027 |
| chr15 | 21421670 | 21421720 |
| chr15 | 23774934 | 23774984 |
| chr15 | 32130203 | 32130253 |
| chr15 | 32270808 | 32270858 |
| chr15 | 34336963 | 34337013 |
| chr15 | 51839385 | 51839435 |
| chr15 | 52895510 | 52895560 |
| chr15 | 53518942 | 53518992 |
| chr15 | 58570041 | 58570091 |
| chr10 | 65836185 | 65836235 |
| chr15 | 59452602 | 59452652 |
| chr15 | 60303393 | 60303443 |
| chr10 | 66899702 | 66899752 |
| chr15 | 67367632 | 67367682 |
| chr10 | 68867417 | 68867467 |
| chr15 | 72889555 | 72889605 |
| chr10 | 69267666 | 69267716 |
| chr15 | 84074086 | 84074136 |
| chr15 | 87252886 | 87252936 |
| chr15 | 87790755 | 87790805 |
| chr15 | 92194416 | 92194466 |
| chr10 | 70416347 | 70416397 |
| chr10 | 70451684 | 70451734 |
| chr16 | 10035785 | 10035835 |
| chr16 | 10358587 | 10358637 |
| chr16 | 12033503 | 12033553 |
| chr10 | 71420401 | 71420451 |
| chr10 | 71420010 | 71420060 |
| chr16 | 14723269 | 14723319 |
| chr16 | 17575064 | 17575114 |
| chr10 | 71729257 | 71729307 |
| chr16 | 28191231 | 28191281 |
| chr16 | 28997042 | 28997092 |
| chr16 | 29738949 | 29738999 |
| chr16 | 29757131 | 29757181 |
| chr16 | 30588126 | 30588176 |
| chr16 | 30593884 | 30593934 |
| chr16 | 30606589 | 30606639 |
| chr16 | 31103357 | 31103407 |
| chr16 | 31332504 | 31332554 |
| chr16 | 35930163 | 35930213 |
| chr16 | 46679745 | 46679795 |
| chr16 | 47185122 | 47185172 |
| chr10 | 73044293 | 73044343 |
| chr16 | 52842471 | 52842521 |
| chr10 | 73067037 | 73067087 |
| chr16 | 56349692 | 56349742 |
| chr16 | 56674389 | 56674439 |
| chr16 | 61053487 | 61053537 |
| chr16 | 62218111 | 62218161 |
| chr16 | 62457890 | 62457940 |
| chr16 | 65674146 | 65674196 |
| chr10 | 73648695 | 73648745 |
| chr10 | 73864084 | 73864134 |
| chr10 | 74008605 | 74008655 |
| chr16 | 70225335 | 70225385 |
| chr10 | 74145595 | 74145645 |
| chr16 | 73913231 | 73913281 |
| chr16 | 77873660 | 77873710 |
| chr16 | 83149237 | 83149287 |
| chr16 | 84155681 | 84155731 |
| chr16 | 87195010 | 87195060 |
| chr17 | 2657301 | 2657351 |
| chr17 | 2895707 | 2895757 |
| chr17 | 6822033 | 6822083 |
| chr10 | 77999698 | 77999748 |
| chr17 | 8172404 | 8172454 |
| chr17 | 8354869 | 8354919 |
| chr17 | 8355875 | 8355925 |
| chr17 | 14006847 | 14006897 |
| chr17 | 14078969 | 14079019 |
| chr17 | 19497434 | 19497484 |
| chr17 | 19611606 | 19611656 |
| chr17 | 21130643 | 21130693 |
| chr17 | 21175835 | 21175885 |
| chr17 | 25792985 | 25793035 |
| chr17 | 26702909 | 26702959 |
| chr17 | 26711366 | 26711416 |
| chr17 | 27309410 | 27309460 |
| chr17 | 28713650 | 28713700 |
| chr17 | 31143847 | 31143897 |
| chr17 | 32241037 | 32241087 |
| chr17 | 32403847 | 32403897 |
| chr17 | 33527835 | 33527885 |
| chr17 | 33876060 | 33876110 |
| chr17 | 37586734 | 37586784 |
| chr17 | 37678539 | 37678589 |

Table 15AR. Repeat Category LTR22

| | | |
|---|---|---|
| chr17 | 39478863 | 39478913 |
| chr17 | 67240401 | 67240451 |
| chr17 | 75355285 | 75355335 |
| chr17 | 80932387 | 80932437 |
| chr18 | 14771469 | 14771519 |
| chr18 | 25255495 | 25255545 |
| chr19 | 5373418 | 5373468 |
| chr19 | 32928882 | 32928932 |
| chr19 | 47892385 | 47892435 |
| chr19 | 47892649 | 47892699 |
| chr19 | 57854025 | 57854075 |
| chr19 | 60096805 | 60096855 |
| chr19 | 62390683 | 62390733 |
| chr1 | 12396086 | 12396136 |
| chr1 | 20330029 | 20330079 |
| chr1 | 94635003 | 94635053 |
| chr1 | 153370515 | 153370565 |
| chr1 | 168460105 | 168460155 |
| chr1 | 190494975 | 190495025 |
| chr1 | 190653142 | 190653192 |
| chr1 | 200686949 | 200686999 |
| chr1 | 223012954 | 223013004 |
| chr21 | 13549685 | 13549735 |
| chr21 | 28243281 | 28243331 |
| chr10 | 9425346 | 9425396 |
| chrX | 36849294 | 36849344 |
| chrX | 63282890 | 63282940 |
| chrX | 101210726 | 101210776 |
| chrX | 118916368 | 118916418 |
| chrX | 140768038 | 140768088 |
| chrX | 145018136 | 145018186 |
| chrX | 145020894 | 145020944 |
| chrX | 147470265 | 147470315 |
| chr2 | 71590282 | 71590332 |
| chr2 | 86758307 | 86758357 |
| chr2 | 109356730 | 109356780 |
| chr2 | 167668621 | 167668671 |
| chr2 | 232168171 | 232168221 |
| chr3 | 111684064 | 111684114 |
| chr3 | 127106844 | 127106894 |
| chr3 | 149564090 | 149564140 |
| chr3 | 171116443 | 171116493 |
| chr4 | 70963685 | 70963735 |
| chr4 | 174343047 | 174343097 |
| chr5 | 26892790 | 26892840 |
| chr5 | 27166782 | 27166832 |
| chr5 | 169858482 | 169858532 |
| chr5 | 178319823 | 178319873 |
| chr11 | 80470448 | 80470498 |
| chr11 | 85368382 | 85368432 |
| chr6 | 56986133 | 56986183 |
| chr6 | 63555999 | 63556049 |
| chr6 | 65010255 | 65010305 |
| chr6 | 69251312 | 69251362 |
| chr6 | 101445672 | 101445722 |
| chr6 | 107116179 | 107116229 |
| chr11 | 95355336 | 95355386 |
| chr6 | 123057462 | 123057512 |
| chr11 | 107813092 | 107813142 |
| chr7 | 39663241 | 39663291 |
| chr7 | 70313913 | 70313963 |
| chr7 | 106587234 | 106587284 |
| chr7 | 125853544 | 125853594 |
| chr7 | 152936797 | 152936847 |
| chr8 | 5664060 | 5664110 |
| chr8 | 33112574 | 33112624 |
| chr8 | 134577670 | 134577720 |
| chr9 | 5221232 | 5221282 |
| chr9 | 6995119 | 6995169 |
| chr9 | 98510946 | 98510996 |
| chr12 | 17328085 | 17328135 |
| chr12 | 39269476 | 39269526 |
| chr12 | 41122714 | 41122764 |
| chr12 | 59258225 | 59258275 |
| chr12 | 87080333 | 87080383 |
| chr12 | 99061198 | 99061248 |
| chr12 | 124945329 | 124945379 |
| chr13 | 18906462 | 18906512 |
| chr13 | 23299820 | 23299870 |
| chr13 | 23309965 | 23310015 |
| chr13 | 69398473 | 69398523 |
| chr10 | 43085538 | 43085588 |
| chr14 | 47264521 | 47264571 |
| chr14 | 78921071 | 78921121 |
| chr15 | 21421670 | 21421720 |
| chr15 | 52895510 | 52895560 |
| chr15 | 87252886 | 87252936 |
| chr16 | 87195010 | 87195060 |

Table 15AS. Repeat Category LTR28

| | | |
|---|---|---|
| chr18 | 20350600 | 20350650 |
| chr18 | 26194652 | 26194702 |
| chr18 | 49404836 | 49404886 |
| chr18 | 61023297 | 61023347 |
| chr1 | 61923197 | 61923247 |
| chr1 | 117104818 | 117104868 |
| chr20 | 33635124 | 33635174 |
| chr21 | 22856440 | 22856490 |
| chr21 | 46870421 | 46870471 |
| chr2 | 136112166 | 136112216 |
| chr11 | 39638352 | 39638402 |
| chr3 | 110264129 | 110264179 |
| chr4 | 45138564 | 45138614 |
| chr4 | 135926320 | 135926370 |
| chr4 | 178997835 | 178997885 |
| chr5 | 7141189 | 7141239 |
| chr5 | 39711015 | 39711065 |
| chr6 | 21574229 | 21574279 |
| chr6 | 141772157 | 141772207 |
| chr6 | 169813003 | 169813053 |
| chr7 | 72965786 | 72965836 |
| chr7 | 104429609 | 104429659 |
| chr8 | 49043557 | 49043607 |
| chr8 | 95246983 | 95247033 |
| chr9 | 84588809 | 84588859 |
| chr12 | 12151599 | 12151649 |
| chr14 | 88414597 | 88414647 |
| chr15 | 32270808 | 32270858 |
| chr17 | 31143847 | 31143897 |
| chr17 | 32241037 | 32241087 |

Table 15AT. Repeat Category LTR29

| | | |
|---|---|---|
| chr17 | 47630768 | 47630818 |
| chr18 | 70350021 | 70350071 |
| chr18 | 70350428 | 70350478 |
| chr1 | 37449091 | 37449141 |
| chr1 | 48031919 | 48031969 |
| chr20 | 35916384 | 35916434 |
| chr22 | 41366555 | 41366605 |
| chrX | 4067913 | 4067963 |
| chrX | 5687488 | 5687538 |
| chrX | 42073388 | 42073438 |
| chrX | 52502437 | 52502487 |
| chrX | 90838663 | 90838713 |
| chrX | 96316297 | 96316347 |
| chrX | 111993667 | 111993717 |
| chr2 | 117982939 | 117982989 |
| chr2 | 117988174 | 117988224 |
| chr2 | 157477584 | 157477634 |
| chr2 | 187308758 | 187308808 |
| chr2 | 228789477 | 228789527 |
| chr3 | 15405995 | 15406045 |
| chr11 | 47633579 | 47633629 |
| chr3 | 54166345 | 54166395 |
| chr3 | 115143053 | 115143103 |
| chr3 | 183829667 | 183829717 |
| chr10 | 13196866 | 13196916 |
| chr4 | 25358912 | 25358962 |
| chr4 | 29979336 | 29979386 |
| chr11 | 68427881 | 68427931 |
| chr11 | 69962547 | 69962597 |
| chr5 | 55659135 | 55659185 |
| chr5 | 78595338 | 78595388 |
| chr5 | 167886974 | 167887024 |
| chr6 | 26074279 | 26074329 |
| chr6 | 27960286 | 27960336 |
| chr6 | 42788729 | 42788779 |
| chr6 | 131688049 | 131688099 |
| chr7 | 48263214 | 48263264 |
| chr7 | 100679139 | 100679189 |
| chr7 | 100679018 | 100679068 |
| chr8 | 56663509 | 56663559 |
| chr8 | 144223084 | 144223134 |
| chr9 | 92764974 | 92765024 |
| chr9 | 132107939 | 132107989 |
| chr12 | 31794390 | 31794440 |
| chr12 | 32456617 | 32456667 |
| chr13 | 26497864 | 26497914 |
| chr13 | 41378934 | 41378984 |
| chr13 | 79762676 | 79762726 |
| chr14 | 29699751 | 29699801 |
| chr10 | 54112943 | 54112993 |
| chr10 | 70416347 | 70416397 |
| chr10 | 70451684 | 70451734 |
| chr16 | 12033503 | 12033553 |
| chr10 | 74145595 | 74145645 |

Table 15AU. Repeat Category LTR30

| | | |
|---|---|---|
| chr18 | 39927387 | 39927437 |
| chr18 | 46176954 | 46177004 |
| chr19 | 467128 | 467178 |
| chr1 | 146964366 | 146964416 |
| chr11 | 299259 | 299309 |
| chrX | 79817252 | 79817302 |
| chrX | 79822965 | 79823015 |
| chrX | 85708031 | 85708081 |
| chrX | 153302858 | 153302908 |
| chr3 | 6084037 | 6084087 |
| chr3 | 99748399 | 99748449 |
| chr3 | 104594079 | 104594129 |
| chr3 | 147057055 | 147057105 |
| chr3 | 155406717 | 155406767 |
| chr3 | 192143533 | 192143583 |
| chr4 | 43335826 | 43335876 |
| chr4 | 63843319 | 63843369 |
| chr4 | 70558024 | 70558074 |
| chr4 | 130841681 | 130841731 |
| chr4 | 136002782 | 136002832 |
| chr4 | 156920679 | 156920729 |
| chr4 | 156921015 | 156921065 |
| chr4 | 179406691 | 179406741 |
| chr5 | 28332941 | 28332991 |
| chr5 | 105284982 | 105285032 |
| chr11 | 82653958 | 82654008 |
| chr11 | 82657767 | 82657817 |
| chr6 | 32636170 | 32636220 |
| chr6 | 38812510 | 38812560 |
| chr6 | 68745751 | 68745801 |
| chr6 | 118984435 | 118984485 |
| chr12 | 14759905 | 14759955 |
| chr10 | 28792964 | 28793014 |
| chr13 | 45483153 | 45483203 |
| chr13 | 58639895 | 58639945 |
| chr13 | 91381860 | 91381910 |
| chr14 | 41705838 | 41705888 |
| chr14 | 41992258 | 41992308 |
| chr14 | 41993984 | 41994034 |
| chr14 | 41994336 | 41994386 |
| chr14 | 41994528 | 41994578 |
| chr14 | 81842247 | 81842297 |
| chr10 | 54243508 | 54243558 |

Table 15AV. Repeat Category LTR33A

| | | |
|---|---|---|
| chr17 | 39094931 | 39094981 |
| chr17 | 41664501 | 41664551 |
| chr17 | 72818570 | 72818620 |
| chr17 | 75886905 | 75886955 |
| chr10 | 92585098 | 92585148 |
| chr18 | 47323488 | 47323538 |
| chr19 | 3517224 | 3517274 |
| chr10 | 101552627 | 101552677 |
| chr19 | 11286591 | 11286641 |
| chr19 | 12944228 | 12944278 |
| chr19 | 46447598 | 46447648 |
| chr19 | 49669421 | 49669471 |
| chr19 | 51685203 | 51685253 |
| chr19 | 52610298 | 52610348 |
| chr19 | 59572389 | 59572439 |
| chr19 | 60008278 | 60008328 |
| chr1 | 23822547 | 23822597 |
| chr1 | 24549677 | 24549727 |
| chr1 | 30805080 | 30805130 |
| chr1 | 32889254 | 32889304 |
| chr1 | 36631178 | 36631228 |
| chr1 | 56885089 | 56885139 |
| chr1 | 58990315 | 58990365 |
| chr1 | 198839665 | 198839715 |
| chr1 | 242244752 | 242244802 |
| chr20 | 46162025 | 46162075 |
| chr20 | 49474599 | 49474649 |
| chr20 | 50250050 | 50250100 |
| chr21 | 26125060 | 26125110 |
| chr22 | 16158992 | 16159042 |
| chr22 | 43338093 | 43338143 |
| chr22 | 46556098 | 46556148 |
| chr11 | 3403708 | 3403758 |
| chr11 | 3403613 | 3403663 |
| chrX | 16506687 | 16506737 |
| chrX | 21552576 | 21552626 |
| chrX | 21552335 | 21552385 |
| chrX | 24638372 | 24638422 |
| chr11 | 6351533 | 6351583 |
| chrX | 45673269 | 45673319 |
| chrX | 75225019 | 75225069 |
| chrX | 117419902 | 117419952 |
| chrX | 121602113 | 121602163 |
| chr11 | 12573813 | 12573863 |
| chrY | 7812612 | 7812662 |
| chr2 | 471654 | 471704 |
| chr2 | 4883365 | 4883415 |
| chr2 | 42143114 | 42143164 |
| chr2 | 55705088 | 55705138 |
| chr2 | 73368258 | 73368308 |
| chr2 | 86019989 | 86020039 |
| chr2 | 114558648 | 114558698 |
| chr11 | 32545341 | 32545391 |
| chr2 | 131707434 | 131707484 |
| chr2 | 139303651 | 139303701 |
| chr2 | 139303577 | 139303627 |
| chr2 | 174023621 | 174023671 |
| chr2 | 181907969 | 181908019 |
| chr2 | 182319616 | 182319666 |
| chr2 | 185233466 | 185233516 |
| chr2 | 189235924 | 189235974 |
| chr11 | 42807628 | 42807678 |
| chr2 | 217674089 | 217674139 |
| chr2 | 220104294 | 220104344 |
| chr2 | 222983630 | 222983680 |
| chr3 | 17765611 | 17765661 |
| chr3 | 39504671 | 39504721 |
| chr3 | 44174416 | 44174466 |
| chr3 | 88711279 | 88711329 |
| chr11 | 59139751 | 59139801 |
| chr3 | 108807770 | 108807820 |
| chr3 | 117234435 | 117234485 |
| chr3 | 131146964 | 131147014 |
| chr3 | 141950363 | 141950413 |
| chr3 | 160592849 | 160592899 |
| chr3 | 195306735 | 195306785 |
| chr3 | 197514149 | 197514199 |
| chr4 | 6083370 | 6083420 |
| chr4 | 6083158 | 6083208 |
| chr4 | 6084408 | 6084458 |
| chr4 | 7575155 | 7575205 |
| chr11 | 64141813 | 64141863 |
| chr4 | 10617774 | 10617824 |
| chr4 | 73345511 | 73345561 |
| chr4 | 79084746 | 79084796 |
| chr4 | 102542122 | 102542172 |
| chr4 | 129892698 | 129892748 |
| chr4 | 137017604 | 137017654 |
| chr4 | 142706444 | 142706494 |
| chr4 | 185961090 | 185961140 |
| chr4 | 189443993 | 189444043 |
| chr4 | 190056762 | 190056812 |
| chr4 | 191170863 | 191170913 |
| chr5 | 6876057 | 6876107 |
| chr5 | 6875834 | 6875884 |
| chr11 | 69720359 | 69720409 |
| chr5 | 30924089 | 30924139 |
| chr5 | 40303154 | 40303204 |
| chr5 | 57823444 | 57823494 |
| chr5 | 57823224 | 57823274 |
| chr5 | 61379468 | 61379518 |
| chr5 | 65013719 | 65013769 |
| chr10 | 14199990 | 14200040 |
| chr5 | 123297030 | 123297080 |
| chr5 | 145264606 | 145264656 |
| chr5 | 148350176 | 148350226 |
| chr11 | 78349472 | 78349522 |
| chr11 | 83181720 | 83181770 |
| chr11 | 83181457 | 83181507 |
| chr6 | 34768366 | 34768416 |
| chr6 | 38811313 | 38811363 |
| chr6 | 39591164 | 39591214 |
| chr6 | 69408524 | 69408574 |
| chr6 | 95628047 | 95628097 |
| chr6 | 127725475 | 127725525 |
| chr6 | 133250655 | 133250705 |
| chr11 | 98499635 | 98499685 |
| chr6 | 160167226 | 160167276 |
| chr11 | 105704033 | 105704083 |
| chr7 | 976211 | 976261 |
| chr7 | 90775449 | 90775499 |
| chr7 | 112986720 | 112986770 |
| chr7 | 150019757 | 150019807 |
| chr7 | 150845493 | 150845543 |
| chr8 | 4741697 | 4741747 |
| chr8 | 26607969 | 26608019 |
| chr8 | 87759115 | 87759165 |
| chr8 | 127366210 | 127366260 |
| chr8 | 141488360 | 141488410 |
| chr9 | 2245219 | 2245269 |
| chr9 | 67095389 | 67095439 |
| chr9 | 69633208 | 69633258 |
| chr9 | 95021737 | 95021787 |
| chr9 | 123538221 | 123538271 |
| chr9 | 127190731 | 127190781 |
| chr12 | 25496369 | 25496419 |
| chr12 | 40264484 | 40264534 |
| chr12 | 45948540 | 45948590 |
| chr12 | 48757183 | 48757233 |
| chr12 | 50843388 | 50843438 |
| chr10 | 27948562 | 27948612 |
| chr12 | 120213417 | 120213467 |
| chr12 | 120527391 | 120527441 |
| chr12 | 124272184 | 124272234 |
| chr12 | 125579367 | 125579417 |
| chr12 | 125579180 | 125579230 |
| chr12 | 127991053 | 127991103 |
| chr12 | 127990901 | 127990951 |
| chr12 | 130131599 | 130131649 |
| chr12 | 130208868 | 130208918 |
| chr12 | 130294882 | 130294932 |
| chr13 | 52748659 | 52748709 |
| chr13 | 81195226 | 81195276 |
| chr13 | 107383651 | 107383701 |
| chr13 | 111156377 | 111156427 |
| chr13 | 111156307 | 111156357 |
| chr14 | 21210758 | 21210808 |
| chr14 | 32420193 | 32420243 |
| chr14 | 45065795 | 45065845 |
| chr14 | 69771401 | 69771451 |
| chr14 | 90711747 | 90711797 |
| chr14 | 94030877 | 94030927 |
| chr14 | 98100114 | 98100164 |
| chr15 | 29265912 | 29265962 |
| chr15 | 57539031 | 57539081 |
| chr15 | 60331970 | 60332020 |
| chr15 | 70711252 | 70711302 |
| chr15 | 92306831 | 92306881 |
| chr15 | 98571503 | 98571553 |
| chr16 | 5143793 | 5143843 |
| chr16 | 26242030 | 26242080 |
| chr16 | 52458502 | 52458552 |
| chr16 | 59177054 | 59177104 |
| chr17 | 12456603 | 12456653 |

Table 15AW. Repeat Category LTR36

| | | |
|---|---|---|
| chr19 | 14240490 | 14240540 |
| chr19 | 46937792 | 46937842 |
| chr1 | 48697941 | 48697991 |
| chr1 | 61875506 | 61875556 |
| chr1 | 107719601 | 107719651 |
| chr1 | 245078798 | 245078848 |
| chr21 | 26174140 | 26174190 |
| chr21 | 42244634 | 42244684 |
| chr21 | 44193736 | 44193786 |
| chr22 | 43038571 | 43038621 |
| chrX | 3270375 | 3270425 |
| chrX | 50138582 | 50138632 |
| chrX | 52018605 | 52018655 |
| chr2 | 47176491 | 47176541 |
| chr2 | 140715701 | 140715751 |
| chr2 | 170909583 | 170909633 |
| chr2 | 232481702 | 232481752 |
| chr3 | 30101478 | 30101528 |
| chr3 | 37380559 | 37380609 |
| chr3 | 52710061 | 52710111 |
| chr3 | 190890346 | 190890396 |
| chr4 | 4534587 | 4534637 |
| chr4 | 41083368 | 41083418 |
| chr4 | 169550949 | 169550999 |
| chr4 | 190074769 | 190074819 |
| chr4 | 190075321 | 190075371 |
| chr4 | 190236932 | 190236982 |
| chr5 | 51210887 | 51210937 |
| chr5 | 177717796 | 177717846 |
| chr6 | 11030566 | 11030616 |
| chr6 | 161105726 | 161105776 |
| chr7 | 21794497 | 21794547 |
| chr11 | 111650778 | 111650828 |
| chr11 | 113671995 | 113672045 |
| chr7 | 96683857 | 96683907 |
| chr10 | 18383321 | 18383371 |
| chr9 | 67117678 | 67117728 |
| chr9 | 92241682 | 92241732 |
| chr9 | 107263773 | 107263823 |
| chr12 | 698565 | 698615 |
| chr12 | 4815043 | 4815093 |
| chr12 | 44254350 | 44254400 |
| chr12 | 44782648 | 44782698 |
| chr12 | 67850931 | 67850981 |
| chr13 | 28427818 | 28427868 |
| chr14 | 53658221 | 53658271 |
| chr15 | 21322944 | 21322994 |
| chr15 | 21324469 | 21324519 |
| chr15 | 43478158 | 43478208 |
| chr16 | 80956986 | 80957036 |

Table 15AX. Repeat Category LTR45B

| | | |
|---|---|---|
| chr17 | 71536945 | 71536995 |
| chr17 | 71536884 | 71536934 |
| chr10 | 97543300 | 97543350 |
| chr19 | 8131777 | 8131827 |
| chr19 | 33626504 | 33626554 |
| chr19 | 49770911 | 49770961 |
| chr1 | 225019719 | 225019769 |
| chr1 | 226282767 | 226282817 |
| chr20 | 56718264 | 56718314 |
| chr21 | 23025494 | 23025544 |
| chr22 | 22587467 | 22587517 |
| chr11 | 5576540 | 5576590 |
| chr2 | 9872410 | 9872460 |
| chr3 | 5991047 | 5991097 |
| chr3 | 105727307 | 105727357 |
| chr3 | 123624609 | 123624659 |
| chr11 | 67192878 | 67192928 |
| chr11 | 67193429 | 67193479 |
| chr5 | 1824488 | 1824538 |
| chr7 | 3824292 | 3824342 |
| chr7 | 6494428 | 6494478 |
| chr7 | 23150968 | 23151018 |
| chr7 | 48076272 | 48076322 |
| chr7 | 65852113 | 65852163 |
| chr7 | 99536327 | 99536377 |
| chr8 | 97427883 | 97427933 |
| chr12 | 27558921 | 27558971 |
| chr12 | 64327527 | 64327577 |
| chr12 | 92537836 | 92537886 |
| chr10 | 30054828 | 30054878 |
| chr13 | 69398473 | 69398523 |
| chr14 | 25339929 | 25339979 |
| chr14 | 75174051 | 75174101 |
| chr10 | 70158564 | 70158614 |

Table 15AY. Repeat Category LTR45C

| | | |
|---|---|---|
| chr17 | 37840905 | 37840955 |
| chr10 | 102632566 | 102632616 |
| chr19 | 60979460 | 60979510 |
| chr1 | 3451431 | 3451481 |
| chr20 | 31184057 | 31184107 |
| chr20 | 47582146 | 47582196 |
| chr21 | 25300956 | 25301006 |
| chr22 | 43765928 | 43765978 |
| chr2 | 104535742 | 104535792 |
| chr3 | 130332571 | 130332621 |
| chr3 | 190371417 | 190371467 |
| chr4 | 141203863 | 141203913 |
| chr5 | 912356 | 912406 |
| chr5 | 31839444 | 31839494 |
| chr5 | 93423230 | 93423280 |
| chr5 | 140676464 | 140676514 |
| chr6 | 14588120 | 14588170 |
| chr6 | 168341544 | 168341594 |
| chr7 | 4360548 | 4360598 |
| chr7 | 71496427 | 71496477 |
| chr7 | 72952260 | 72952310 |
| chr7 | 151044429 | 151044479 |
| chr9 | 27697310 | 27697360 |
| chr9 | 33161868 | 33161918 |
| chr12 | 97976341 | 97976391 |
| chr12 | 116694534 | 116694584 |
| chr12 | 131850894 | 131850944 |
| chr13 | 44068136 | 44068186 |
| chr13 | 44074326 | 44074376 |
| chr13 | 62785215 | 62785265 |
| chr13 | 63526352 | 63526402 |
| chr15 | 25391384 | 25391434 |
| chr16 | 16885017 | 16885067 |
| chr17 | 4767595 | 4767645 |

Table 15AZ. Repeat Category LTR46

| | | |
|---|---|---|
| chr17 | 42482324 | 42482374 |
| chr17 | 79087451 | 79087501 |
| chr18 | 9687455 | 9687505 |
| chr19 | 14725069 | 14725119 |
| chr19 | 37743375 | 37743425 |
| chr19 | 40052996 | 40053046 |
| chr19 | 54663365 | 54663415 |
| chr19 | 62565411 | 62565461 |
| chr1 | 1014584 | 1014634 |
| chr1 | 39580304 | 39580354 |
| chr1 | 157338607 | 157338657 |
| chr22 | 43833974 | 43834024 |
| chrX | 73291486 | 73291536 |
| chrX | 138790465 | 138790515 |
| chrX | 139479401 | 139479451 |
| chrX | 141495800 | 141495850 |
| chrY | 3357464 | 3357514 |
| chr2 | 89449668 | 89449718 |
| chr2 | 89731498 | 89731548 |
| chr2 | 99476209 | 99476259 |
| chr2 | 201932701 | 201932751 |
| chr2 | 201933265 | 201933315 |
| chr4 | 33366577 | 33366627 |
| chr11 | 68535566 | 68535616 |
| chr11 | 95921253 | 95921303 |
| chr11 | 95928706 | 95928756 |
| chr11 | 110566207 | 110566257 |
| chr11 | 115903812 | 115903862 |
| chr7 | 77958420 | 77958470 |
| chr7 | 97993177 | 97993227 |
| chr8 | 143865047 | 143865097 |
| chr9 | 5490959 | 5491009 |
| chrX | 139366249 | 139366299 |
| chrX | 139366339 | 139366389 |
| chr12 | 83807340 | 83807390 |
| chr12 | 131976388 | 131976438 |
| chr14 | 102380087 | 102380137 |
| chr16 | 75495898 | 75495948 |
| chr17 | 33866426 | 33866476 |

Table 15BA. Repeat Category LTR47A

| | | |
|---|---|---|
| chr10 | 88437821 | 88437871 |
| chr18 | 9748034 | 9748084 |
| chr18 | 35417984 | 35418034 |
| chr18 | 59263778 | 59263828 |
| chr18 | 74012476 | 74012526 |
| chr19 | 42188319 | 42188369 |
| chr19 | 44459047 | 44459097 |
| chr19 | 54929211 | 54929261 |
| chr1 | 141717898 | 141717948 |
| chr1 | 198193523 | 198193573 |
| chr1 | 227269693 | 227269743 |
| chr1 | 230159655 | 230159705 |
| chr10 | 134251419 | 134251469 |
| chr22 | 37765871 | 37765921 |
| chr22 | 41298475 | 41298525 |
| chr22 | 41371678 | 41371728 |
| chrX | 46996197 | 46996247 |
| chrX | 109550892 | 109550942 |
| chrX | 134676196 | 134676246 |
| chr2 | 797109 | 797159 |
| chr2 | 60925446 | 60925496 |
| chr11 | 38491161 | 38491211 |
| chr2 | 207240404 | 207240454 |
| chr2 | 223890228 | 223890278 |
| chr2 | 225818525 | 225818575 |
| chr2 | 230461139 | 230461189 |
| chr3 | 45208409 | 45208459 |
| chr3 | 51861954 | 51862004 |
| chr3 | 108172717 | 108172767 |
| chr11 | 66511904 | 66511954 |
| chr4 | 187916651 | 187916701 |
| chr5 | 115169987 | 115170037 |
| chr5 | 125209670 | 125209720 |
| chr5 | 131461571 | 131461621 |
| chr5 | 139632166 | 139632216 |
| chr11 | 76173418 | 76173468 |
| chr6 | 26253667 | 26253717 |
| chr6 | 31075637 | 31075687 |
| chr6 | 163388692 | 163388742 |
| chr7 | 70761298 | 70761348 |
| chr7 | 74987338 | 74987388 |
| chr7 | 156640825 | 156640875 |
| chr7 | 157162002 | 157162052 |
| chr8 | 20768318 | 20768368 |
| chr8 | 144295522 | 144295572 |
| chr8 | 144862249 | 144862299 |
| chr9 | 103838681 | 103838731 |
| chr12 | 19661140 | 19661190 |
| chr12 | 129642208 | 129642258 |
| chr13 | 25892907 | 25892957 |
| chr13 | 61001781 | 61001831 |
| chr13 | 108918272 | 108918322 |
| chr14 | 63572535 | 63572585 |
| chr15 | 32696064 | 32696114 |
| chr16 | 14285640 | 14285690 |
| chr16 | 68066143 | 68066193 |
| chr17 | 326904 | 326954 |
| chr17 | 4759712 | 4759762 |
| chr17 | 14626539 | 14626589 |

Table 15BC. Repeat Category LTR48

| | | |
|---|---|---|
| chr10 | 85992953 | 85993003 |
| chr17 | 65990673 | 65990723 |
| chr17 | 66301781 | 66301831 |
| chr17 | 66303100 | 66303150 |
| chr17 | 78936307 | 78936357 |
| chr18 | 20250868 | 20250918 |
| chr18 | 27658717 | 27658767 |
| chr18 | 59067516 | 59067566 |
| chr19 | 13714376 | 13714426 |
| chr19 | 45491551 | 45491601 |
| chr19 | 45493111 | 45493161 |
| chr19 | 45495804 | 45495854 |
| chr19 | 45497455 | 45497505 |
| chr19 | 45501660 | 45501710 |
| chr10 | 113903581 | 113903631 |
| chr1 | 3358006 | 3358056 |
| chr1 | 7845181 | 7845231 |
| chr1 | 9407954 | 9408004 |
| chr1 | 34754419 | 34754469 |
| chr1 | 104788206 | 104788256 |
| chr1 | 112707296 | 112707346 |
| chr1 | 184499204 | 184499254 |
| chr1 | 198815657 | 198815707 |
| chr1 | 206360577 | 206360627 |
| chr1 | 206452325 | 206452375 |
| chr1 | 206452217 | 206452267 |
| chr1 | 217268950 | 217269000 |
| chr1 | 225638433 | 225638483 |
| chr1 | 244930893 | 244930943 |
| chr20 | 31064004 | 31064054 |
| chr21 | 17927081 | 17927131 |
| chrX | 8072165 | 8072215 |
| chrX | 27702543 | 27702593 |
| chrX | 41365260 | 41365310 |
| chrX | 52732420 | 52732470 |
| chrX | 107578791 | 107578841 |
| chrX | 109434157 | 109434207 |
| chrX | 111816968 | 111817018 |
| chrX | 112045793 | 112045843 |
| chr2 | 2876140 | 2876190 |
| chr2 | 11318389 | 11318439 |
| chr11 | 20152409 | 20152459 |
| chr2 | 44263336 | 44263386 |
| chr2 | 48875420 | 48875470 |
| chr2 | 58315050 | 58315100 |
| chr2 | 65022818 | 65022868 |
| chr2 | 78066424 | 78066474 |
| chr11 | 30840018 | 30840068 |
| chr2 | 113435500 | 113435550 |
| chr2 | 125260480 | 125260530 |
| chr2 | 164855114 | 164855164 |
| chr2 | 189343679 | 189343729 |
| chr2 | 231470809 | 231470859 |
| chr3 | 16667365 | 16667415 |
| chr3 | 18862739 | 18862789 |
| chr3 | 77443094 | 77443144 |
| chr3 | 137795862 | 137795912 |
| chr3 | 164445099 | 164445149 |
| chr4 | 57533616 | 57533666 |
| chr4 | 84865911 | 84865961 |
| chr4 | 91007165 | 91007215 |
| chr4 | 97922037 | 97922087 |
| chr4 | 121114209 | 121114259 |
| chr4 | 130058115 | 130058165 |
| chr4 | 152236221 | 152236271 |
| chr4 | 160186307 | 160186357 |
| chr5 | 5135821 | 5135871 |
| chr5 | 68232572 | 68232622 |
| chr5 | 76302357 | 76302407 |
| chr5 | 105213189 | 105213239 |
| chr5 | 130134327 | 130134377 |
| chr5 | 137773859 | 137773909 |
| chr5 | 141301187 | 141301237 |
| chr5 | 177667040 | 177667090 |
| chr5 | 178389283 | 178389333 |
| chr6 | 14494024 | 14494074 |
| chr6 | 15071326 | 15071376 |
| chr6 | 26142481 | 26142531 |
| chr6 | 27702588 | 27702638 |
| chr6 | 27929112 | 27929162 |
| chr6 | 53116649 | 53116699 |
| chr6 | 107367209 | 107367259 |
| chr6 | 136228076 | 136228126 |
| chr6 | 142138041 | 142138091 |
| chr6 | 158787186 | 158787236 |
| chr7 | 84577091 | 84577141 |
| chr11 | 116672036 | 116672086 |
| chr7 | 99037161 | 99037211 |
| chr7 | 110845006 | 110845056 |
| chr7 | 154112101 | 154112151 |
| chr7 | 154111917 | 154111967 |
| chr8 | 6546819 | 6546869 |
| chr8 | 8972811 | 8972861 |
| chr8 | 29257775 | 29257825 |
| chr8 | 39782594 | 39782644 |
| chr8 | 62129993 | 62130043 |
| chr8 | 116360637 | 116360687 |
| chr8 | 120939691 | 120939741 |
| chr9 | 11090325 | 11090375 |
| chr9 | 32805406 | 32805456 |
| chr9 | 41213594 | 41213644 |
| chr9 | 71390395 | 71390445 |
| chr9 | 80454672 | 80454722 |
| chr9 | 86605059 | 86605109 |
| chr9 | 92767059 | 92767109 |
| chr9 | 127702445 | 127702495 |
| chr12 | 5570223 | 5570273 |
| chr12 | 127937241 | 127937291 |
| chr13 | 24903239 | 24903289 |
| chr13 | 32121780 | 32121830 |
| chr13 | 43799802 | 43799852 |
| chr10 | 41984579 | 41984629 |
| chr13 | 72292849 | 72292899 |
| chr13 | 72292668 | 72292718 |
| chr14 | 66064461 | 66064511 |
| chr14 | 79420434 | 79420484 |
| chr15 | 32378171 | 32378221 |
| chr15 | 57422872 | 57422922 |
| chr15 | 61909551 | 61909601 |
| chr15 | 89107239 | 89107289 |
| chr15 | 89773235 | 89773285 |
| chr16 | 10337191 | 10337241 |
| chr16 | 10358587 | 10358637 |
| chr16 | 14373254 | 14373304 |
| chr16 | 18950563 | 18950613 |
| chr16 | 60019869 | 60019919 |
| chr10 | 73434940 | 73434990 |

Table 15BD. Repeat Category LTR48B

| | | |
|---|---|---|
| chr17 | 65990673 | 65990723 |
| chr18 | 20250868 | 20250918 |
| chr18 | 27658717 | 27658767 |
| chr1 | 34754419 | 34754469 |
| chr1 | 112707296 | 112707346 |
| chr1 | 198815657 | 198815707 |
| chr1 | 206360577 | 206360627 |
| chr1 | 225638433 | 225638483 |
| chr1 | 244930893 | 244930943 |
| chrX | 8072165 | 8072215 |
| chrX | 27702543 | 27702593 |
| chrX | 41365260 | 41365310 |
| chrX | 52732420 | 52732470 |
| chrX | 112045793 | 112045843 |
| chr2 | 2876140 | 2876190 |
| chr2 | 48875420 | 48875470 |
| chr2 | 65022818 | 65022868 |
| chr2 | 125260480 | 125260530 |
| chr2 | 164855114 | 164855164 |
| chr2 | 189343679 | 189343729 |
| chr3 | 18862739 | 18862789 |
| chr3 | 77443094 | 77443144 |
| chr3 | 164445099 | 164445149 |
| chr4 | 57533616 | 57533666 |
| chr4 | 84865911 | 84865961 |
| chr4 | 121114209 | 121114259 |
| chr4 | 152236221 | 152236271 |
| chr4 | 160186307 | 160186357 |
| chr5 | 5135821 | 5135871 |
| chr5 | 68232572 | 68232622 |
| chr5 | 141301187 | 141301237 |
| chr6 | 14494024 | 14494074 |
| chr6 | 15071326 | 15071376 |
| chr6 | 27929112 | 27929162 |
| chr6 | 53116649 | 53116699 |
| chr6 | 136228076 | 136228126 |
| chr6 | 142138041 | 142138091 |
| chr8 | 8972811 | 8972861 |
| chr8 | 29257775 | 29257825 |
| chr8 | 39782594 | 39782644 |
| chr8 | 116360637 | 116360687 |
| chr8 | 120939691 | 120939741 |
| chr9 | 86605059 | 86605109 |
| chr9 | 127702445 | 127702495 |
| chr12 | 5570223 | 5570273 |
| chr12 | 127937241 | 127937291 |
| chr13 | 24903239 | 24903289 |
| chr13 | 32121780 | 32121830 |
| chr13 | 43799802 | 43799852 |
| chr14 | 79420434 | 79420484 |
| chr15 | 57422872 | 57422922 |
| chr15 | 89107239 | 89107289 |
| chr16 | 10337191 | 10337241 |
| chr16 | 10358587 | 10358637 |
| chr16 | 14373254 | 14373304 |
| chr16 | 60019869 | 60019919 |

Table 15BE. Repeat Category LTR5

| | | |
|---|---|---|
| chr17 | 38212629 | 38212679 |
| chr17 | 38950887 | 38950937 |
| chr17 | 41916256 | 41916306 |
| chr17 | 42159421 | 42159471 |
| chr17 | 43596128 | 43596178 |
| chr17 | 47210829 | 47210879 |
| chr17 | 47213443 | 47213493 |
| chr17 | 47215152 | 47215202 |
| chr17 | 53176089 | 53176139 |
| chr17 | 57841643 | 57841693 |
| chr17 | 58087489 | 58087539 |
| chr17 | 58119386 | 58119436 |
| chr10 | 86582408 | 86582458 |
| chr17 | 73229072 | 73229122 |
| chr17 | 74174621 | 74174671 |
| chr17 | 74455813 | 74455863 |
| chr17 | 74657289 | 74657339 |
| chr17 | 74657221 | 74657271 |
| chr17 | 77260496 | 77260546 |
| chr17 | 77261469 | 77261519 |
| chr10 | 91128615 | 91128665 |
| chr18 | 7262926 | 7262976 |
| chr18 | 11935591 | 11935641 |
| chr18 | 22759338 | 22759388 |
| chr18 | 26785077 | 26785127 |
| chr18 | 27643414 | 27643464 |
| chr18 | 29034028 | 29034078 |
| chr18 | 30087080 | 30087130 |
| chr18 | 33032404 | 33032454 |
| chr18 | 33036041 | 33036091 |
| chr18 | 37016758 | 37016808 |
| chr18 | 41919995 | 41920045 |
| chr18 | 43598178 | 43598228 |
| chr18 | 51448579 | 51448629 |
| chr18 | 62892958 | 62893008 |
| chr18 | 63999808 | 63999858 |
| chr18 | 75923806 | 75923856 |
| chr10 | 100731341 | 100731391 |
| chr19 | 3956579 | 3956629 |
| chr10 | 101245038 | 101245088 |
| chr19 | 8358173 | 8358223 |
| chr19 | 9179990 | 9180040 |
| chr19 | 9558728 | 9558778 |
| chr19 | 9597460 | 9597510 |
| chr19 | 9760951 | 9761001 |
| chr19 | 11583758 | 11583808 |
| chr19 | 12099896 | 12099946 |
| chr19 | 12173944 | 12173994 |
| chr19 | 12229801 | 12229851 |
| chr19 | 12232261 | 12232311 |
| chr19 | 12457118 | 12457168 |
| chr19 | 12582109 | 12582159 |
| chr19 | 13636202 | 13636252 |
| chr19 | 14608060 | 14608110 |
| chr19 | 18374953 | 18375003 |
| chr19 | 19675509 | 19675559 |
| chr19 | 19927249 | 19927299 |
| chr19 | 19928049 | 19928099 |
| chr19 | 20053946 | 20053996 |
| chr19 | 22550554 | 22550604 |
| chr19 | 39014444 | 39014494 |
| chr19 | 39097157 | 39097207 |
| chr19 | 39210799 | 39210849 |
| chr19 | 39298718 | 39298768 |
| chr19 | 40104065 | 40104115 |
| chr19 | 40312519 | 40312569 |
| chr19 | 41416208 | 41416258 |
| chr19 | 41983444 | 41983494 |
| chr19 | 42297826 | 42297876 |
| chr19 | 42434816 | 42434866 |
| chr19 | 42927372 | 42927422 |
| chr19 | 43051386 | 43051436 |
| chr19 | 43814405 | 43814455 |
| chr19 | 44661844 | 44661894 |
| chr19 | 45157085 | 45157135 |
| chr19 | 46020142 | 46020192 |
| chr19 | 47224570 | 47224620 |
| chr19 | 48096167 | 48096217 |
| chr19 | 48528031 | 48528081 |
| chr19 | 49476942 | 49476992 |
| chr19 | 49790227 | 49790277 |
| chr19 | 50064723 | 50064773 |
| chr19 | 50064941 | 50064991 |
| chr19 | 54023193 | 54023243 |
| chr19 | 54084423 | 54084473 |
| chr19 | 54128856 | 54128906 |
| chr19 | 54409083 | 54409133 |
| chr19 | 54572471 | 54572521 |
| chr19 | 54573662 | 54573712 |
| chr19 | 54912714 | 54912764 |
| chr19 | 55201725 | 55201775 |
| chr19 | 55850247 | 55850297 |
| chr10 | 112819591 | 112819641 |
| chr19 | 56990359 | 56990409 |
| chr19 | 56990162 | 56990212 |
| chr19 | 57101744 | 57101794 |
| chr19 | 57223274 | 57223324 |
| chr19 | 57534103 | 57534153 |
| chr19 | 57583805 | 57583855 |
| chr19 | 57678883 | 57678933 |
| chr19 | 57715328 | 57715378 |
| chr19 | 57936032 | 57936082 |
| chr19 | 58067902 | 58067952 |
| chr19 | 58131094 | 58131144 |
| chr19 | 58139364 | 58139414 |
| chr19 | 58139848 | 58139898 |
| chr19 | 58524305 | 58524355 |
| chr19 | 58655200 | 58655250 |
| chr19 | 58663290 | 58663340 |
| chr19 | 58693704 | 58693754 |
| chr19 | 60147270 | 60147320 |
| chr19 | 60264098 | 60264148 |
| chr19 | 60921908 | 60921958 |
| chr19 | 61889467 | 61889517 |
| chr19 | 62908476 | 62908526 |
| chr19 | 62979314 | 62979364 |
| chr19 | 63014267 | 63014317 |
| chr19 | 63041136 | 63041186 |
| chr19 | 63081082 | 63081132 |
| chr1 | 4082911 | 4082961 |
| chr1 | 8772419 | 8772469 |
| chr1 | 9768472 | 9768522 |
| chr1 | 9922438 | 9922488 |
| chr1 | 10197485 | 10197535 |
| chr1 | 11660596 | 11660646 |
| chr1 | 14824498 | 14824548 |
| chr1 | 16841185 | 16841235 |
| chr1 | 16899241 | 16899291 |
| chr1 | 16898581 | 16898631 |
| chr1 | 24164941 | 24164991 |
| chr1 | 24820455 | 24820505 |
| chr1 | 25518926 | 25518976 |
| chr1 | 26741394 | 26741444 |
| chr1 | 29352010 | 29352060 |
| chr1 | 29374465 | 29374515 |
| chr10 | 6308540 | 6308590 |
| chr1 | 30505961 | 30506011 |
| chr1 | 30875004 | 30875054 |
| chr1 | 31132939 | 31132989 |
| chr1 | 32421253 | 32421303 |
| chr1 | 34925638 | 34925688 |
| chr10 | 121231194 | 121231244 |
| chr1 | 38893291 | 38893341 |
| chr1 | 39594422 | 39594472 |
| chr1 | 40452176 | 40452226 |
| chr1 | 50867854 | 50867904 |
| chr10 | 123353996 | 123354046 |
| chr10 | 124214281 | 124214331 |
| chr1 | 70297151 | 70297201 |
| chr1 | 72392831 | 72392881 |
| chr1 | 79591418 | 79591468 |
| chr1 | 87979260 | 87979310 |
| chr1 | 88999425 | 88999475 |
| chr1 | 89000532 | 89000582 |
| chr1 | 91136245 | 91136295 |
| chr1 | 99722492 | 99722542 |
| chr1 | 99764281 | 99764331 |
| chr10 | 125933926 | 125933976 |
| chr10 | 6878733 | 6878783 |
| chr1 | 109607654 | 109607704 |
| chr1 | 109609184 | 109609234 |
| chr1 | 109704713 | 109704763 |
| chr1 | 142422815 | 142422865 |
| chr1 | 143848963 | 143849013 |
| chr1 | 144219839 | 144219889 |
| chr1 | 149644103 | 149644153 |
| chr10 | 127203590 | 127203640 |
| chr1 | 157877337 | 157877387 |
| chr1 | 158133402 | 158133452 |
| chr1 | 158502271 | 158502321 |
| chr1 | 158599498 | 158599548 |
| chr10 | 530890 | 530940 |
| chr1 | 163766609 | 163766659 |
| chr1 | 166138524 | 166138574 |
| chr1 | 170683770 | 170683820 |
| chr1 | 179933430 | 179933480 |
| chr1 | 184282640 | 184282690 |
| chr1 | 185214345 | 185214395 |
| chr1 | 210113833 | 210113883 |
| chr1 | 210268275 | 210268325 |
| chr1 | 225300517 | 225300567 |
| chr1 | 232206612 | 232206662 |
| chr1 | 236392854 | 236392904 |
| chr10 | 132083157 | 132083207 |
| chr10 | 132158267 | 132158317 |
| chr1 | 243193452 | 243193502 |
| chr20 | 4078369 | 4078419 |
| chr20 | 4080502 | 4080552 |
| chr20 | 4082818 | 4082868 |
| chr20 | 5724614 | 5724664 |
| chr20 | 5860895 | 5860945 |
| chr20 | 11386842 | 11386892 |
| chr20 | 14508301 | 14508351 |
| chr20 | 14607546 | 14607596 |
| chr10 | 133294432 | 133294482 |
| chr20 | 20871044 | 20871094 |
| chr20 | 22982692 | 22982742 |
| chr20 | 36282036 | 36282086 |
| chr20 | 37267363 | 37267413 |
| chr20 | 37268504 | 37268554 |
| chr20 | 45783368 | 45783418 |
| chr20 | 47194089 | 47194139 |
| chr20 | 49811822 | 49811872 |
| chr10 | 134140180 | 134140230 |
| chr20 | 53116671 | 53116721 |
| chr20 | 54833663 | 54833713 |
| chr20 | 62620801 | 62620851 |
| chr20 | 63017220 | 63017270 |
| chr20 | 63018381 | 63018431 |
| chr21 | 14575322 | 14575372 |
| chr11 | 177578 | 177628 |
| chr11 | 191665 | 191715 |
| chr21 | 17960114 | 17960164 |
| chr21 | 17988104 | 17988154 |
| chr21 | 18418060 | 18418110 |
| chr21 | 26125060 | 26125110 |
| chr21 | 33290009 | 33290059 |
| chr21 | 37536121 | 37536171 |
| chr21 | 37535965 | 37536015 |
| chr21 | 37823869 | 37823919 |
| chr21 | 41713057 | 41713107 |
| chr21 | 41714151 | 41714201 |
| chr21 | 43461083 | 43461133 |
| chr21 | 44558906 | 44558956 |
| chr21 | 44928959 | 44929009 |
| chr22 | 15773725 | 15773775 |
| chr22 | 15915572 | 15915622 |
| chr22 | 15914400 | 15914450 |
| chr22 | 16538232 | 16538282 |
| chr22 | 17266672 | 17266722 |
| chr22 | 19773175 | 19773225 |
| chr22 | 22203765 | 22203815 |
| chr22 | 22573782 | 22573832 |
| chr22 | 22574875 | 22574925 |
| chr22 | 22623362 | 22623412 |
| chr22 | 30888012 | 30888062 |
| chr22 | 30936393 | 30936443 |
| chr22 | 32255216 | 32255266 |
| chr22 | 34619426 | 34619476 |
| chr22 | 34848163 | 34848213 |
| chr22 | 34934904 | 34934954 |
| chr22 | 37651766 | 37651816 |
| chr11 | 2456544 | 2456594 |
| chr22 | 40350458 | 40350508 |
| chr22 | 45121278 | 45121328 |
| chr22 | 48338196 | 48338246 |
| chr11 | 3441612 | 3441662 |
| chrX | 2435691 | 2435741 |
| chrX | 4916362 | 4916412 |
| chr10 | 10581271 | 10581321 |
| chrX | 18360369 | 18360419 |
| chr11 | 5071229 | 5071279 |
| chrX | 22687586 | 22687636 |
| chrX | 25598216 | 25598266 |
| chrX | 30001173 | 30001223 |
| chrX | 37680962 | 37681012 |
| chrX | 39219479 | 39219529 |
| chrX | 40325645 | 40325695 |
| chrX | 43457122 | 43457172 |
| chrX | 43622446 | 43622496 |
| chrX | 50318105 | 50318155 |
| chrX | 50474127 | 50474177 |
| chrX | 54829011 | 54829061 |
| chrX | 56576723 | 56576773 |
| chrX | 56912124 | 56912174 |
| chrX | 60828851 | 60828901 |
| chrX | 61595332 | 61595382 |
| chrX | 62389251 | 62389301 |
| chrX | 70820814 | 70820864 |
| chrX | 76112402 | 76112452 |
| chrX | 85999669 | 85999719 |
| chrX | 86586306 | 86586356 |
| chrX | 90186963 | 90187013 |
| chrX | 106392702 | 106392752 |
| chrX | 112966741 | 112966791 |
| chr11 | 10178787 | 10178837 |
| chrX | 120574243 | 120574293 |
| chrX | 121949911 | 121949961 |
| chrX | 123239105 | 123239155 |
| chrX | 128110094 | 128110144 |
| chrX | 132777701 | 132777751 |
| chrX | 136122200 | 136122250 |
| chrX | 147342261 | 147342311 |
| chrX | 147345673 | 147345723 |
| chrX | 147681116 | 147681166 |
| chrX | 150826296 | 150826346 |
| chrY | 14224373 | 14224423 |
| chr2 | 803011 | 803061 |
| chr2 | 1386924 | 1386974 |
| chr2 | 1386770 | 1386820 |
| chr2 | 1653587 | 1653637 |
| chr2 | 7590938 | 7590988 |
| chr2 | 7590758 | 7590808 |
| chr2 | 8855284 | 8855334 |
| chr2 | 10375995 | 10376045 |
| chr2 | 14721153 | 14721203 |
| chr2 | 20542197 | 20542247 |
| chr11 | 18303058 | 18303108 |
| chr2 | 26213571 | 26213621 |
| chr2 | 26948363 | 26948413 |
| chr2 | 27367431 | 27367481 |
| chr2 | 30293756 | 30293806 |
| chr2 | 32476162 | 32476212 |
| chr2 | 33276483 | 33276533 |
| chr2 | 35009255 | 35009305 |
| chr2 | 38480197 | 38480247 |
| chr2 | 43051909 | 43051959 |
| chr2 | 47968310 | 47968360 |
| chr2 | 54520656 | 54520706 |
| chr11 | 22936248 | 22936298 |
| chr11 | 23078326 | 23078376 |
| chr2 | 62129354 | 62129404 |
| chr2 | 62629146 | 62629196 |
| chr2 | 64271607 | 64271657 |
| chr2 | 73321932 | 73321982 |
| chr2 | 76030916 | 76030966 |
| chr2 | 86019756 | 86019806 |
| chr2 | 86019989 | 86020039 |
| chr2 | 89068246 | 89068296 |
| chr2 | 91236795 | 91236845 |
| chr2 | 105748503 | 105748553 |
| chr2 | 113527152 | 113527202 |
| chr2 | 113787792 | 113787842 |
| chr2 | 114612118 | 114612168 |
| chr2 | 120233747 | 120233797 |
| chr2 | 120382380 | 120382430 |
| chr2 | 122028884 | 122028934 |
| chr2 | 128302671 | 128302721 |
| chr2 | 128777913 | 128777963 |
| chr2 | 131890722 | 131890772 |
| chr2 | 133227915 | 133227965 |
| chr2 | 135936087 | 135936137 |
| chr2 | 142367141 | 142367191 |
| chr2 | 153086980 | 153087030 |
| chr2 | 154890591 | 154890641 |
| chr2 | 162226587 | 162226637 |
| chr2 | 167164919 | 167164969 |
| chr2 | 181977849 | 181977899 |
| chr2 | 187588348 | 187588398 |
| chr2 | 199868786 | 199868836 |
| chr2 | 199944855 | 199944905 |
| chr2 | 201899765 | 201899815 |
| chr2 | 202207405 | 202207455 |
| chr2 | 204125383 | 204125433 |
| chr2 | 219163193 | 219163243 |
| chr2 | 221389386 | 221389436 |
| chr2 | 228789477 | 228789527 |
| chr2 | 229435141 | 229435191 |
| chr2 | 230248061 | 230248111 |
| chr2 | 231240337 | 231240387 |
| chr2 | 231240075 | 231240125 |
| chr2 | 232645112 | 232645162 |
| chr2 | 232710689 | 232710739 |
| chr2 | 233460964 | 233461014 |
| chr2 | 235569011 | 235569061 |
| chr2 | 238710706 | 238710756 |
| chr3 | 5097995 | 5098045 |
| chr3 | 5099285 | 5099335 |
| chr3 | 9663170 | 9663220 |
| chr3 | 9870157 | 9870207 |
| chr3 | 12748811 | 12748861 |
| chr3 | 23452238 | 23452288 |
| chr11 | 47760530 | 47760580 |
| chr3 | 38137127 | 38137177 |
| chr3 | 38138628 | 38138678 |
| chr3 | 39421348 | 39421398 |
| chr3 | 39780790 | 39780840 |
| chr3 | 42868634 | 42868684 |
| chr3 | 43261356 | 43261406 |
| chr3 | 44435106 | 44435156 |
| chr3 | 44514297 | 44514347 |
| chr3 | 45758782 | 45758832 |
| chr3 | 46081060 | 46081110 |
| chr3 | 48115327 | 48115377 |
| chr11 | 55560301 | 55560351 |
| chr3 | 65803559 | 65803609 |
| chr3 | 71949171 | 71949221 |
| chr3 | 72058973 | 72059023 |
| chr3 | 75363945 | 75363995 |
| chr3 | 75521334 | 75521384 |
| chr3 | 81050160 | 81050210 |
| chr11 | 58678998 | 58679048 |
| chr11 | 58680134 | 58680184 |
| chr3 | 97591732 | 97591782 |
| chr3 | 100459853 | 100459903 |
| chr3 | 101376436 | 101376486 |
| chr3 | 105532960 | 105533010 |
| chr11 | 59918930 | 59918980 |
| chr3 | 111407124 | 111407174 |
| chr3 | 114075108 | 114075158 |
| chr3 | 114880044 | 114880094 |
| chr11 | 60255915 | 60255965 |
| chr11 | 60302788 | 60302838 |
| chr3 | 125782712 | 125782762 |
| chr3 | 126931118 | 126931168 |
| chr3 | 129731695 | 129731745 |
| chr11 | 60924835 | 60924885 |
| chr3 | 131098184 | 131098234 |
| chr3 | 131162886 | 131162936 |
| chr3 | 131162790 | 131162840 |
| chr3 | 147561904 | 147561954 |
| chr3 | 152718256 | 152718306 |
| chr3 | 157121064 | 157121114 |
| chr11 | 61738520 | 61738570 |
| chr11 | 61870485 | 61870535 |
| chr11 | 61914174 | 61914224 |
| chr11 | 61925022 | 61925072 |
| chr3 | 178363070 | 178363120 |
| chr3 | 180532846 | 180532896 |
| chr3 | 181796123 | 181796173 |
| chr3 | 184560025 | 184560075 |
| chr11 | 62403018 | 62403068 |
| chr3 | 188263334 | 188263384 |
| chr3 | 196025398 | 196025448 |
| chr3 | 197658103 | 197658153 |
| chr3 | 198989054 | 198989104 |
| chr4 | 226019 | 226069 |
| chr4 | 1985442 | 1985492 |
| chr4 | 2759844 | 2759894 |
| chr4 | 3354055 | 3354105 |
| chr4 | 3353869 | 3353919 |
| chr4 | 3689621 | 3689671 |
| chr4 | 4043501 | 4043551 |
| chr4 | 4052072 | 4052122 |
| chr4 | 5827767 | 5827817 |
| chr11 | 64114644 | 64114694 |
| chr4 | 9503863 | 9503913 |
| chr4 | 9879847 | 9879897 |
| chr11 | 64566820 | 64566870 |
| chr4 | 29986235 | 29986285 |
| chr4 | 34395984 | 34396034 |
| chr4 | 35477560 | 35477610 |
| chr4 | 40159666 | 40159716 |
| chr4 | 40800479 | 40800529 |
| chr4 | 48203041 | 48203091 |
| chr4 | 60345528 | 60345578 |
| chr4 | 65225603 | 65225653 |
| chr4 | 69150697 | 69150747 |
| chr4 | 78482962 | 78483012 |
| chr4 | 83293080 | 83293130 |
| chr4 | 89881624 | 89881674 |
| chr4 | 97374024 | 97374074 |
| chr4 | 97904176 | 97904226 |
| chr4 | 109108881 | 109108931 |
| chr4 | 130089597 | 130089647 |
| chr4 | 136918281 | 136918331 |
| chr4 | 146139065 | 146139115 |
| chr4 | 157885104 | 157885154 |
| chr10 | 13686047 | 13686097 |
| chr4 | 162157466 | 162157516 |
| chr10 | 13685834 | 13685884 |
| chr4 | 163918000 | 163918050 |
| chr4 | 166520004 | 166520054 |
| chr4 | 166572656 | 166572706 |
| chr4 | 167592424 | 167592474 |
| chr4 | 173599653 | 173599703 |
| chr4 | 176018754 | 176018804 |
| chr4 | 183347153 | 183347203 |
| chr4 | 185343383 | 185343433 |
| chr4 | 186743116 | 186743166 |
| chr4 | 188512088 | 188512138 |
| chr4 | 188984139 | 188984189 |
| chr4 | 190556677 | 190556727 |
| chr4 | 190972775 | 190972825 |
| chr4 | 191131919 | 191131969 |
| chr5 | 250030 | 250080 |
| chr5 | 1648794 | 1648844 |
| chr5 | 1860820 | 1860870 |
| chr5 | 2955624 | 2955674 |
| chr5 | 4977684 | 4977734 |
| chr5 | 10254793 | 10254843 |
| chr5 | 10254521 | 10254571 |
| chr5 | 11315236 | 11315286 |
| chr5 | 16676943 | 16676993 |
| chr5 | 18156976 | 18157026 |
| chr5 | 19367338 | 19367388 |
| chr5 | 21337684 | 21337734 |
| chr5 | 37717404 | 37717454 |
| chr5 | 38816080 | 38816130 |
| chr5 | 42967259 | 42967309 |
| chr5 | 43021322 | 43021372 |
| chr5 | 55339846 | 55339896 |
| chr5 | 55469507 | 55469557 |
| chr5 | 76936282 | 76936332 |
| chr5 | 78289643 | 78289693 |
| chr5 | 78969219 | 78969269 |
| chr5 | 79491046 | 79491096 |
| chr5 | 79491922 | 79491972 |
| chr11 | 70912291 | 70912341 |
| chr5 | 92187827 | 92187877 |
| chr5 | 92867637 | 92867687 |
| chr5 | 105671781 | 105671831 |
| chr5 | 105739154 | 105739204 |
| chr11 | 71624551 | 71624601 |
| chr11 | 71624085 | 71624135 |
| chr11 | 71624161 | 71624211 |
| chr5 | 118476844 | 118476894 |
| chr11 | 72232937 | 72232987 |
| chr5 | 130301407 | 130301457 |
| chr5 | 131697694 | 131697744 |
| chr5 | 132218624 | 132218674 |
| chr5 | 133709258 | 133709308 |
| chr11 | 73068908 | 73068958 |
| chr5 | 141669062 | 141669112 |
| chr5 | 149173636 | 149173686 |
| chr5 | 153565323 | 153565373 |
| chr5 | 154045071 | 154045121 |
| chr5 | 154101609 | 154101659 |
| chr5 | 156075016 | 156075066 |
| chr5 | 159610319 | 159610369 |
| chr5 | 159737184 | 159737234 |
| chr5 | 161114538 | 161114588 |
| chr5 | 163845088 | 163845138 |
| chr10 | 14752833 | 14752883 |
| chr5 | 169017888 | 169017938 |
| chr5 | 169756391 | 169756441 |
| chr5 | 171467209 | 171467259 |
| chr5 | 172586454 | 172586504 |
| chr5 | 174982399 | 174982449 |
| chr5 | 177596414 | 177596464 |
| chr5 | 179758671 | 179758721 |
| chr5 | 180365018 | 180365068 |
| chr5 | 180659178 | 180659228 |
| chr5 | 180803497 | 180803547 |
| chr6 | 2524571 | 2524621 |
| chr6 | 2854841 | 2854891 |
| chr6 | 5585635 | 5585685 |
| chr6 | 5585498 | 5585548 |
| chr6 | 10574982 | 10575032 |
| chr6 | 10693170 | 10693220 |
| chr6 | 19785228 | 19785278 |
| chr11 | 80225328 | 80225378 |
| chr6 | 22581376 | 22581426 |
| chr6 | 24770751 | 24770801 |
| chr6 | 25629663 | 25629713 |
| chr6 | 26313993 | 26314043 |
| chr6 | 26864200 | 26864250 |
| chr6 | 27672853 | 27672903 |
| chr6 | 27901757 | 27901807 |
| chr6 | 28759257 | 28759307 |
| chr6 | 28763699 | 28763749 |
| chr6 | 28945433 | 28945483 |
| chr6 | 28946011 | 28946061 |
| chr6 | 32555394 | 32555444 |
| chr6 | 33580245 | 33580295 |
| chr6 | 35344242 | 35344292 |
| chr6 | 36649234 | 36649284 |
| chr6 | 37367387 | 37367437 |
| chr6 | 42909285 | 42909335 |
| chr6 | 42917246 | 42917296 |
| chr6 | 44775175 | 44775225 |
| chr6 | 64832193 | 64832243 |
| chr6 | 69185129 | 69185179 |
| chr11 | 90428685 | 90428735 |
| chr6 | 78431752 | 78431802 |
| chr6 | 83084521 | 83084571 |
| chr11 | 91901264 | 91901314 |
| chr6 | 90596642 | 90596692 |
| chr11 | 93343104 | 93343154 |
| chr6 | 110222353 | 110222403 |
| chr6 | 110228704 | 110228754 |
| chr6 | 111185814 | 111185864 |
| chr6 | 111408743 | 111408793 |
| chr6 | 127541536 | 127541586 |
| chr6 | 135380018 | 135380068 |
| chr6 | 135634057 | 135634107 |
| chr6 | 151533824 | 151533874 |
| chr6 | 153016984 | 153017034 |
| chr11 | 100636870 | 100636920 |
| chr11 | 101103329 | 101103379 |
| chr6 | 159399357 | 159399407 |
| chr6 | 160413126 | 160413176 |
| chr6 | 160414437 | 160414487 |
| chr6 | 162298701 | 162298751 |
| chr11 | 102791860 | 102791910 |
| chr6 | 165823852 | 165823902 |
| chr11 | 102927867 | 102927917 |
| chr11 | 102935242 | 102935292 |
| chr6 | 166853530 | 166853580 |
| chr6 | 170650606 | 170650656 |
| chr7 | 1859001 | 1859051 |
| chr7 | 4752613 | 4752663 |
| chr7 | 5368745 | 5368795 |
| chr7 | 21190287 | 21190337 |
| chr7 | 22234162 | 22234212 |
| chr7 | 23196919 | 23196969 |
| chr7 | 23539081 | 23539131 |
| chr7 | 27524638 | 27524688 |
| chr7 | 32297009 | 32297059 |
| chr7 | 32313423 | 32313473 |
| chr7 | 44637395 | 44637445 |
| chr7 | 47770789 | 47770839 |
| chr7 | 49411593 | 49411643 |
| chr7 | 50991381 | 50991431 |
| chr7 | 50991154 | 50991204 |
| chr7 | 65495540 | 65495590 |
| chr7 | 74994612 | 74994662 |
| chr7 | 96559666 | 96559716 |
| chr7 | 98641772 | 98641822 |
| chr7 | 100680721 | 100680771 |
| chr11 | 117681634 | 117681684 |
| chr7 | 112338636 | 112338686 |
| chr7 | 116070953 | 116071003 |
| chr7 | 130411018 | 130411068 |
| chr11 | 118447704 | 118447754 |
| chr7 | 136415656 | 136415706 |
| chr7 | 136980756 | 136980806 |
| chr7 | 136980696 | 136980746 |
| chr7 | 137891411 | 137891461 |
| chr7 | 139660490 | 139660540 |
| chr7 | 140863241 | 140863291 |
| chr7 | 148156215 | 148156265 |
| chr7 | 148774038 | 148774088 |
| chr7 | 148777194 | 148777244 |
| chr7 | 150115663 | 150115713 |
| chr7 | 150115776 | 150115826 |
| chr7 | 151839062 | 151839112 |
| chr7 | 153639294 | 153639344 |
| chr7 | 156133528 | 156133578 |
| chr7 | 156304969 | 156305019 |
| chr7 | 156502968 | 156503018 |
| chr8 | 600706 | 600756 |
| chr8 | 5054762 | 5054812 |
| chr8 | 7392473 | 7392523 |
| chr8 | 7667950 | 7668000 |
| chr8 | 7668627 | 7668677 |
| chr8 | 8101666 | 8101716 |
| chr8 | 13448334 | 13448384 |
| chr8 | 14045686 | 14045736 |
| chr8 | 15273499 | 15273549 |
| chr8 | 20651626 | 20651676 |
| chr11 | 122688337 | 122688387 |
| chr8 | 38059585 | 38059635 |
| chr8 | 47359859 | 47359909 |
| chr8 | 50364187 | 50364237 |
| chr8 | 52962599 | 52962649 |
| chr8 | 53727703 | 53727753 |
| chr8 | 59634378 | 59634428 |
| chr8 | 59635828 | 59635878 |
| chr8 | 60104683 | 60104733 |
| chr8 | 62813507 | 62813557 |
| chr8 | 74147741 | 74147791 |
| chr8 | 94534426 | 94534476 |
| chr8 | 98818862 | 98818912 |
| chr8 | 100912384 | 100912434 |
| chr8 | 103399565 | 103399615 |
| chr8 | 107546488 | 107546538 |
| chr8 | 113432643 | 113432693 |
| chr8 | 120886933 | 120886983 |
| chr8 | 127261747 | 127261797 |
| chr8 | 129953432 | 129953482 |
| chr8 | 134532339 | 134532389 |
| chr8 | 134532409 | 134532459 |
| chr8 | 140034628 | 140034678 |
| chr8 | 140200399 | 140200449 |
| chr8 | 144469340 | 144469390 |
| chr8 | 146083556 | 146083606 |
| chr8 | 146180661 | 146180711 |
| chr8 | 146257865 | 146257915 |
| chr9 | 6085716 | 6085766 |
| chr9 | 6686338 | 6686388 |
| chr9 | 26705109 | 26705159 |
| chr9 | 30678947 | 30678997 |
| chr9 | 40608390 | 40608440 |
| chr9 | 71159213 | 71159263 |
| chr9 | 73352841 | 73352891 |
| chr9 | 74665595 | 74665645 |
| chr9 | 75105168 | 75105218 |
| chr9 | 79383344 | 79383394 |
| chr9 | 82439898 | 82439948 |
| chr9 | 89417021 | 89417071 |
| chr9 | 90957962 | 90958012 |
| chr9 | 93373976 | 93374026 |
| chr9 | 94514620 | 94514670 |
| chr9 | 94515309 | 94515359 |
| chr9 | 111348528 | 111348578 |
| chr9 | 111518356 | 111518406 |
| chr9 | 112717357 | 112717407 |
| chr9 | 125035429 | 125035479 |
| chr9 | 126811035 | 126811085 |
| chr9 | 126995797 | 126995847 |
| chr9 | 129509269 | 129509319 |
| chr9 | 135033112 | 135033162 |
| chr9 | 135049672 | 135049722 |
| chr9 | 135369186 | 135369236 |
| chr12 | 5516406 | 5516456 |
| chr12 | 6778219 | 6778269 |
| chr12 | 6866209 | 6866259 |
| chr12 | 7692419 | 7692469 |
| chr12 | 8263998 | 8264048 |
| chr12 | 8680450 | 8680500 |
| chr12 | 9855911 | 9855961 |
| chr12 | 10615103 | 10615153 |
| chr12 | 12977866 | 12977916 |
| chr12 | 14348933 | 14348983 |
| chr12 | 20117288 | 20117338 |
| chr12 | 21689893 | 21689943 |
| chr12 | 27818870 | 27818920 |
| chr12 | 34259795 | 34259845 |
| chr12 | 34259704 | 34259754 |
| chr12 | 40423906 | 40423956 |
| chr12 | 41011150 | 41011200 |
| chr12 | 41068802 | 41068852 |
| chr12 | 49805389 | 49805439 |
| chr12 | 49805481 | 49805531 |
| chr12 | 51192764 | 51192814 |
| chr12 | 54748754 | 54748804 |
| chr12 | 55503152 | 55503202 |
| chr12 | 57016996 | 57017046 |
| chr12 | 59869047 | 59869097 |
| chr12 | 66208371 | 66208421 |
| chr12 | 67362124 | 67362174 |
| chr12 | 67837052 | 67837102 |
| chr12 | 71688735 | 71688785 |
| chr10 | 28878208 | 28878258 |
| chr12 | 84365534 | 84365584 |
| chr12 | 85414207 | 85414257 |
| chr12 | 89271681 | 89271731 |
| chr12 | 89669396 | 89669446 |
| chr12 | 91776408 | 91776458 |
| chr12 | 91970617 | 91970667 |
| chr12 | 95771213 | 95771263 |
| chr10 | 1293416 | 1293466 |
| chr10 | 32653876 | 32653926 |
| chr12 | 121673137 | 121673187 |
| chr12 | 123029942 | 123029992 |
| chr12 | 126057309 | 126057359 |
| chr12 | 127061202 | 127061252 |
| chr12 | 127492584 | 127492634 |
| chr12 | 127727856 | 127727906 |
| chr10 | 34951328 | 34951378 |
| chr10 | 35065409 | 35065459 |
| chr10 | 35390026 | 35390076 |
| chr13 | 18294370 | 18294420 |
| chr13 | 18493512 | 18493562 |
| chr13 | 18944891 | 18944941 |
| chr13 | 19776102 | 19776152 |
| chr13 | 19849293 | 19849343 |
| chr13 | 21185979 | 21186029 |
| chr13 | 26311853 | 26311903 |
| chr13 | 41412887 | 41412937 |
| chr13 | 47972108 | 47972158 |
| chr13 | 52639040 | 52639090 |
| chr13 | 61237284 | 61237334 |
| chr13 | 65366572 | 65366622 |
| chr13 | 66171172 | 66171222 |
| chr13 | 68851070 | 68851120 |
| chr13 | 69562460 | 69562510 |
| chr13 | 73091831 | 73091881 |
| chr13 | 78143115 | 78143165 |
| chr13 | 86205023 | 86205073 |
| chr10 | 43117707 | 43117757 |
| chr13 | 93619085 | 93619135 |
| chr13 | 95850463 | 95850513 |
| chr13 | 96404922 | 96404972 |
| chr10 | 43231649 | 43231699 |
| chr10 | 43231302 | 43231352 |
| chr10 | 43540235 | 43540285 |
| chr13 | 108796648 | 108796698 |
| chr13 | 110546628 | 110546678 |
| chr13 | 111609992 | 111610042 |
| chr13 | 111611289 | 111611339 |
| chr13 | 111982820 | 111982870 |
| chr13 | 112385790 | 112385840 |
| chr13 | 112865395 | 112865445 |
| chr14 | 22469566 | 22469616 |
| chr14 | 24252092 | 24252142 |
| chr14 | 36822964 | 36823014 |
| chr14 | 48745203 | 48745253 |
| chr14 | 49011794 | 49011844 |
| chr14 | 51200028 | 51200078 |
| chr14 | 53209126 | 53209176 |
| chr10 | 50899856 | 50899906 |
| chr14 | 68991832 | 68991882 |
| chr14 | 68991716 | 68991766 |
| chr14 | 69625718 | 69625768 |
| chr14 | 71485098 | 71485148 |
| chr14 | 76091376 | 76091426 |
| chr14 | 76117748 | 76117798 |
| chr14 | 76152163 | 76152213 |
| chr14 | 82004118 | 82004168 |
| chr14 | 85477331 | 85477381 |
| chr14 | 88283794 | 88283844 |
| chr14 | 93082450 | 93082500 |
| chr14 | 93082268 | 93082318 |
| chr14 | 94900977 | 94901027 |
| chr14 | 101472452 | 101472502 |
| chr14 | 101889761 | 101889811 |
| chr14 | 103661657 | 103661707 |
| chr14 | 103711185 | 103711235 |
| chr14 | 104171193 | 104171243 |
| chr15 | 23816087 | 23816137 |
| chr15 | 24614617 | 24614667 |
| chr15 | 26142158 | 26142208 |
| chr15 | 27557906 | 27557956 |
| chr15 | 39623078 | 39623128 |
| chr15 | 42808318 | 42808368 |
| chr15 | 51492229 | 51492279 |
| chr15 | 68383158 | 68383208 |
| chr15 | 71182443 | 71182493 |
| chr10 | 68955555 | 68955605 |
| chr15 | 72894311 | 72894361 |
| chr15 | 76229398 | 76229448 |
| chr15 | 76229450 | 76229500 |
| chr15 | 79444909 | 79444959 |
| chr15 | 82552541 | 82552591 |
| chr15 | 89247277 | 89247327 |
| chr15 | 100135410 | 100135460 |
| chr16 | 799691 | 799741 |
| chr16 | 1465411 | 1465461 |
| chr16 | 3093650 | 3093700 |
| chr16 | 3550533 | 3550583 |
| chr10 | 3057865 | 3057915 |
| chr16 | 10396311 | 10396361 |
| chr16 | 17738062 | 17738112 |
| chr16 | 18937337 | 18937387 |
| chr16 | 21054833 | 21054883 |
| chr16 | 21193947 | 21193997 |
| chr16 | 25043347 | 25043397 |
| chr16 | 48243212 | 48243262 |
| chr16 | 64233038 | 64233088 |
| chr16 | 68134896 | 68134946 |
| chr16 | 68134692 | 68134742 |
| chr16 | 75628703 | 75628753 |
| chr16 | 83692170 | 83692220 |
| chr16 | 84715560 | 84715610 |
| chr16 | 89027035 | 89027085 |
| chr16 | 89051255 | 89051305 |
| chr16 | 89859686 | 89859736 |
| chr17 | 1928094 | 1928144 |
| chr10 | 76313405 | 76313455 |
| chr10 | 76313622 | 76313672 |
| chr10 | 76314057 | 76314107 |
| chr17 | 2985591 | 2985641 |
| chr17 | 6279672 | 6279722 |
| chr17 | 6751001 | 6751051 |
| chr17 | 6976142 | 6976192 |
| chr17 | 8429697 | 8429747 |
| chr17 | 9563207 | 9563257 |
| chr17 | 11194088 | 11194138 |
| chr17 | 11218555 | 11218605 |
| chr17 | 11711370 | 11711420 |
| chr17 | 15376515 | 15376565 |
| chr17 | 15375773 | 15375823 |
| chr17 | 15582313 | 15582363 |
| chr17 | 15582374 | 15582424 |
| chr17 | 19570267 | 19570317 |
| chr17 | 19585172 | 19585222 |
| chr17 | 29067387 | 29067437 |
| chr17 | 29173707 | 29173757 |
| chr17 | 30329244 | 30329294 |

Table 15BF. Repeat Category LTR51

| | | |
|---|---|---|
| chr19 | 14608060 | 14608110 |
| chr19 | 39097157 | 39097207 |
| chr1 | 9768472 | 9768522 |
| chr1 | 16899241 | 16899291 |
| chr1 | 16898581 | 16898631 |
| chr10 | 530890 | 530940 |
| chr20 | 37267363 | 37267413 |
| chr20 | 37268504 | 37268554 |
| chr20 | 53116671 | 53116721 |
| chr21 | 37823869 | 37823919 |
| chr22 | 30936393 | 30936443 |
| chrX | 4916362 | 4916412 |
| chr2 | 142367141 | 142367191 |
| chr4 | 183347153 | 183347203 |
| chr4 | 186743116 | 186743166 |
| chr5 | 92187827 | 92187877 |
| chr11 | 71624551 | 71624601 |
| chr11 | 71624085 | 71624135 |
| chr11 | 71624161 | 71624211 |
| chr11 | 72232937 | 72232987 |
| chr5 | 133709258 | 133709308 |
| chr10 | 14752833 | 14752883 |
| chr5 | 174982399 | 174982449 |
| chr6 | 25629663 | 25629713 |
| chr6 | 26313993 | 26314043 |
| chr6 | 35344242 | 35344292 |
| chr6 | 69185129 | 69185179 |
| chr7 | 49411593 | 49411643 |
| chr12 | 9855911 | 9855961 |
| chr12 | 12977866 | 12977916 |
| chr12 | 127061202 | 127061252 |
| chr13 | 95850463 | 95850513 |
| chr15 | 23816087 | 23816137 |
| chr16 | 18937337 | 18937387 |

Table 15BG. Repeat Category LTR52

| | | |
|---|---|---|
| chr17 | 41916256 | 41916306 |
| chr17 | 42159421 | 42159471 |
| chr17 | 47210829 | 47210879 |
| chr17 | 47213443 | 47213493 |
| chr17 | 47215152 | 47215202 |
| chr17 | 53176089 | 53176139 |
| chr18 | 26785077 | 26785127 |
| chr18 | 63999808 | 63999858 |
| chr10 | 100731341 | 100731391 |
| chr19 | 12173944 | 12173994 |
| chr19 | 12229801 | 12229851 |
| chr19 | 12232261 | 12232311 |
| chr19 | 12457118 | 12457168 |
| chr19 | 12582109 | 12582159 |
| chr19 | 19675509 | 19675559 |
| chr19 | 42927372 | 42927422 |
| chr19 | 55201725 | 55201775 |
| chr10 | 112819591 | 112819641 |
| chr19 | 57583805 | 57583855 |
| chr1 | 30505961 | 30506011 |
| chr1 | 38893291 | 38893341 |
| chr20 | 4078369 | 4078419 |
| chr20 | 4080502 | 4080552 |
| chr20 | 4082818 | 4082868 |
| chr22 | 48338196 | 48338246 |
| chrX | 2435691 | 2435741 |
| chrX | 50474127 | 50474177 |
| chrX | 120574243 | 120574293 |
| chrX | 123239105 | 123239155 |
| chrX | 128110094 | 128110144 |
| chr2 | 14721153 | 14721203 |
| chr2 | 76030916 | 76030966 |
| chr2 | 153086980 | 153087030 |
| chr2 | 231240337 | 231240387 |
| chr2 | 231240075 | 231240125 |
| chr3 | 72058973 | 72059023 |
| chr3 | 105532960 | 105533010 |
| chr3 | 114880044 | 114880094 |
| chr4 | 35477560 | 35477610 |
| chr4 | 89881624 | 89881674 |
| chr4 | 97374024 | 97374074 |
| chr4 | 166520004 | 166520054 |
| chr4 | 188984139 | 188984189 |
| chr5 | 76936282 | 76936332 |
| chr5 | 105739154 | 105739204 |
| chr6 | 64832193 | 64832243 |
| chr6 | 83084521 | 83084571 |
| chr11 | 100636870 | 100636920 |
| chr7 | 4752613 | 4752663 |
| chr8 | 5054762 | 5054812 |
| chr8 | 13448334 | 13448384 |
| chr8 | 50364187 | 50364237 |
| chr8 | 113432643 | 113432693 |
| chr9 | 30678947 | 30678997 |
| chr9 | 90957962 | 90958012 |
| chr12 | 40423906 | 40423956 |
| chr12 | 51192764 | 51192814 |
| chr12 | 85414207 | 85414257 |
| chr13 | 66171172 | 66171222 |
| chr13 | 68851070 | 68851120 |
| chr13 | 69562460 | 69562510 |
| chr13 | 78143115 | 78143165 |
| chr10 | 43231649 | 43231699 |
| chr10 | 43231302 | 43231352 |
| chr15 | 24614617 | 24614667 |
| chr16 | 3093650 | 3093700 |
| chr16 | 64233038 | 64233088 |
| chr17 | 15582313 | 15582363 |
| chr17 | 15582374 | 15582424 |

Table 15BH. Repeat Category LTR54B

| | | |
|---|---|---|
| chr17 | 38212629 | 38212679 |
| chr17 | 38950887 | 38950937 |
| chr19 | 11583758 | 11583808 |
| chr19 | 12099896 | 12099946 |
| chr19 | 39210799 | 39210849 |
| chr19 | 48096167 | 48096217 |
| chr1 | 179933430 | 179933480 |
| chr1 | 210113833 | 210113883 |
| chr1 | 210268275 | 210268325 |
| chr1 | 225300517 | 225300567 |
| chr20 | 14508301 | 14508351 |
| chr20 | 14607546 | 14607596 |
| chr11 | 177578 | 177628 |
| chr22 | 30888012 | 30888062 |
| chr11 | 5071229 | 5071279 |
| chrX | 30001173 | 30001223 |
| chrX | 40325645 | 40325695 |
| chrX | 43622446 | 43622496 |
| chrX | 86586306 | 86586356 |
| chrX | 90186963 | 90187013 |
| chrX | 132777701 | 132777751 |
| chrX | 147681116 | 147681166 |
| chr2 | 133227915 | 133227965 |
| chr2 | 235569011 | 235569061 |
| chr3 | 44514297 | 44514347 |
| chr3 | 65803559 | 65803609 |
| chr3 | 129731695 | 129731745 |
| chr4 | 78482962 | 78483012 |
| chr5 | 2955624 | 2955674 |
| chr5 | 11315236 | 11315286 |
| chr5 | 78969219 | 78969269 |
| chr5 | 159610319 | 159610369 |
| chr5 | 159737184 | 159737234 |
| chr5 | 163845088 | 163845138 |
| chr6 | 2524571 | 2524621 |
| chr6 | 22581376 | 22581426 |
| chr6 | 110228704 | 110228754 |
| chr11 | 102927867 | 102927917 |
| chr7 | 96559666 | 96559716 |
| chr7 | 100680721 | 100680771 |
| chr9 | 6686338 | 6686388 |
| chr9 | 89417021 | 89417071 |
| chr12 | 34259795 | 34259845 |
| chr12 | 34259704 | 34259754 |
| chr12 | 67362124 | 67362174 |
| chr12 | 95771213 | 95771263 |
| chr13 | 18493512 | 18493562 |
| chr14 | 51200028 | 51200078 |
| chr14 | 101889761 | 101889811 |
| chr15 | 27557906 | 27557956 |
| chr16 | 3550533 | 3550583 |
| chr16 | 21054833 | 21054883 |
| chr16 | 84715560 | 84715610 |

Table 15BI. Repeat Category LTR56

| | | |
|---|---|---|
| chr17 | 58087489 | 58087539 |
| chr10 | 86582408 | 86582458 |
| chr17 | 73229072 | 73229122 |
| chr18 | 33032404 | 33032454 |
| chr18 | 33036041 | 33036091 |
| chr19 | 50064723 | 50064773 |
| chr19 | 50064941 | 50064991 |
| chr19 | 54023193 | 54023243 |
| chr1 | 16841185 | 16841235 |
| chr10 | 124214281 | 124214331 |
| chr1 | 91136245 | 91136295 |
| chr1 | 109704713 | 109704763 |
| chr1 | 142422815 | 142422865 |
| chr1 | 236392854 | 236392904 |
| chr10 | 133294432 | 133294482 |
| chr20 | 20871044 | 20871094 |
| chr10 | 134140180 | 134140230 |
| chr21 | 37536121 | 37536171 |
| chr21 | 37535965 | 37536015 |
| chr22 | 17266672 | 17266722 |
| chrX | 61595332 | 61595382 |
| chrX | 76112402 | 76112452 |
| chr2 | 1653587 | 1653637 |
| chr11 | 18303058 | 18303108 |
| chr2 | 47968310 | 47968360 |
| chr2 | 62629146 | 62629196 |
| chr2 | 64271607 | 64271657 |
| chr2 | 114612118 | 114612168 |
| chr2 | 120382380 | 120382430 |
| chr2 | 131890722 | 131890772 |
| chr2 | 162226587 | 162226637 |
| chr2 | 232710689 | 232710739 |
| chr3 | 46081060 | 46081110 |
| chr3 | 71949171 | 71949221 |
| chr3 | 125782712 | 125782762 |
| chr11 | 60924835 | 60924885 |
| chr3 | 152718256 | 152718306 |
| chr4 | 2759844 | 2759894 |
| chr4 | 3689621 | 3689671 |
| chr4 | 40159666 | 40159716 |
| chr4 | 191131919 | 191131969 |
| chr5 | 19367338 | 19367388 |
| chr5 | 55339846 | 55339896 |
| chr5 | 118476844 | 118476894 |
| chr11 | 73068908 | 73068958 |
| chr5 | 154101609 | 154101659 |
| chr6 | 5585635 | 5585685 |
| chr6 | 5585498 | 5585548 |
| chr6 | 44775175 | 44775225 |
| chr6 | 111185814 | 111185864 |
| chr7 | 5368745 | 5368795 |
| chr7 | 32313423 | 32313473 |
| chr7 | 50991381 | 50991431 |
| chr7 | 50991154 | 50991204 |
| chr7 | 130411018 | 130411068 |
| chr7 | 137891411 | 137891461 |
| chr7 | 148774038 | 148774088 |
| chr7 | 148777194 | 148777244 |
| chr7 | 151839062 | 151839112 |
| chr8 | 20651626 | 20651676 |
| chr8 | 59635828 | 59635878 |
| chr8 | 62813507 | 62813557 |
| chr8 | 140200399 | 140200449 |
| chr9 | 40608390 | 40608440 |
| chr9 | 94514620 | 94514670 |
| chr9 | 94515309 | 94515359 |
| chr9 | 126811035 | 126811085 |
| chr12 | 7692419 | 7692469 |
| chr12 | 20117288 | 20117338 |
| chr12 | 54748754 | 54748804 |
| chr10 | 32653876 | 32653926 |
| chr10 | 35065409 | 35065459 |
| chr13 | 18294370 | 18294420 |
| chr13 | 18944891 | 18944941 |
| chr13 | 111982820 | 111982870 |
| chr13 | 112385790 | 112385840 |
| chr14 | 103661657 | 103661707 |
| chr15 | 72894311 | 72894361 |
| chr16 | 1465411 | 1465461 |
| chr16 | 10396311 | 10396361 |
| chr16 | 21193947 | 21193997 |
| chr17 | 2985591 | 2985641 |
| chr17 | 8429697 | 8429747 |

Table 15BJ. Repeat Category LTR5A

| | | |
|---|---|---|
| chr19 | 58524305 | 58524355 |
| chr19 | 60264098 | 60264148 |
| chr22 | 15915572 | 15915622 |
| chr22 | 15914400 | 15914450 |
| chr11 | 3441612 | 3441662 |
| chr11 | 23078326 | 23078376 |
| chr2 | 135936087 | 135936137 |
| chr3 | 75521334 | 75521384 |
| chr3 | 131162886 | 131162936 |
| chr3 | 131162790 | 131162840 |
| chr4 | 4043501 | 4043551 |
| chr4 | 4052072 | 4052122 |
| chr4 | 9503863 | 9503913 |
| chr4 | 29986235 | 29986285 |
| chr4 | 157885104 | 157885154 |
| chr4 | 185343383 | 185343433 |
| chr5 | 250030 | 250080 |
| chr5 | 154045071 | 154045121 |
| chr6 | 160413126 | 160413176 |
| chr6 | 160414437 | 160414487 |
| chr8 | 7392473 | 7392523 |
| chr8 | 7667950 | 7668000 |
| chr8 | 7668627 | 7668677 |
| chr8 | 8101666 | 8101716 |
| chr11 | 122688337 | 122688387 |
| chr8 | 47359859 | 47359909 |
| chr8 | 146257865 | 146257915 |
| chr12 | 8263998 | 8264048 |
| chr12 | 55503152 | 55503202 |
| chr13 | 112865395 | 112865445 |
| chr14 | 22469566 | 22469616 |
| chr14 | 53209126 | 53209176 |
| chr14 | 93082450 | 93082500 |
| chr14 | 93082268 | 93082318 |

Table 15BK. Repeat Category LTR65

| | | |
|---|---|---|
| chr17 | 58679296 | 58679346 |
| chr19 | 2366379 | 2366429 |
| chr19 | 47054260 | 47054310 |
| chr19 | 53468256 | 53468306 |
| chr1 | 29924046 | 29924096 |
| chr1 | 147063494 | 147063544 |
| chr1 | 233536900 | 233536950 |
| chr20 | 30778714 | 30778764 |
| chr20 | 55704857 | 55704907 |
| chr11 | 4662221 | 4662271 |
| chrX | 46268177 | 46268227 |
| chr2 | 37398453 | 37398503 |
| chr2 | 37398863 | 37398913 |
| chr2 | 101578855 | 101578905 |
| chr3 | 111395145 | 111395195 |
| chr11 | 62385503 | 62385553 |
| chr4 | 68572670 | 68572720 |
| chr6 | 24755917 | 24755967 |
| chr6 | 26546561 | 26546611 |
| chr6 | 26546667 | 26546717 |
| chr6 | 74081386 | 74081436 |
| chr7 | 22963657 | 22963707 |
| chr8 | 42973385 | 42973435 |
| chr8 | 98611214 | 98611264 |
| chr8 | 124012294 | 124012344 |
| chr12 | 63193804 | 63193854 |
| chr12 | 108975571 | 108975621 |
| chr10 | 32350313 | 32350363 |
| chr13 | 98944739 | 98944789 |
| chr14 | 32956461 | 32956511 |
| chr16 | 62102874 | 62102924 |
| chr17 | 3843718 | 3843768 |
| chr17 | 3844580 | 3844630 |
| chr17 | 21121035 | 21121085 |

Table 15BL. Repeat Category LTR66

| | | |
|---|---|---|
| chr17 | 42525744 | 42525794 |
| chr17 | 42575356 | 42575406 |
| chr17 | 42576034 | 42576084 |
| chr19 | 9217326 | 9217376 |
| chr19 | 19834257 | 19834307 |
| chr19 | 20038058 | 20038108 |
| chr19 | 20044959 | 20045009 |
| chr19 | 20125977 | 20126027 |
| chr10 | 121195738 | 121195788 |
| chr1 | 111132469 | 111132519 |
| chr1 | 111132109 | 111132159 |
| chr1 | 212089602 | 212089652 |
| chr1 | 223698589 | 223698639 |
| chr1 | 223705206 | 223705256 |
| chr1 | 223705042 | 223705092 |
| chr20 | 19627186 | 19627236 |
| chr22 | 16142004 | 16142054 |
| chrX | 4151370 | 4151420 |
| chrX | 40185787 | 40185837 |
| chr2 | 108839271 | 108839321 |
| chr11 | 56165381 | 56165431 |
| chr4 | 262844 | 262894 |
| chr4 | 439031 | 439081 |
| chr4 | 187976047 | 187976097 |
| chr5 | 40875544 | 40875594 |
| chr5 | 99999729 | 99999779 |
| chr8 | 27970116 | 27970166 |
| chr8 | 51820559 | 51820609 |
| chr9 | 21971252 | 21971302 |
| chr11 | 133048199 | 133048249 |
| chr9 | 86137877 | 86137927 |
| chr9 | 86137286 | 86137336 |
| chr9 | 110878440 | 110878490 |
| chr12 | 63200484 | 63200534 |
| chr12 | 125738149 | 125738199 |
| chr13 | 88556100 | 88556150 |
| chr14 | 80803824 | 80803874 |
| chr14 | 101717064 | 101717114 |
| chr16 | 20720540 | 20720590 |
| chr16 | 20721230 | 20721280 |
| chr16 | 23721734 | 23721784 |
| chr16 | 29044602 | 29044652 |
| chr16 | 75369771 | 75369821 |
| chr16 | 75370295 | 75370345 |

Table 15BM. Repeat Category LTR6A

| | | |
|---|---|---|
| chr17 | 76865531 | 76865581 |
| chr17 | 80811888 | 80811938 |
| chr17 | 80848148 | 80848198 |
| chr10 | 93457709 | 93457759 |
| chr19 | 6607293 | 6607343 |
| chr19 | 6612172 | 6612222 |
| chr19 | 17387418 | 17387468 |
| chr19 | 20334390 | 20334440 |
| chr19 | 21671956 | 21672006 |
| chr19 | 21831147 | 21831197 |
| chr19 | 38178985 | 38179035 |
| chr19 | 54025367 | 54025417 |
| chr19 | 55945378 | 55945428 |
| chr19 | 61550226 | 61550276 |
| chr19 | 63090568 | 63090618 |
| chr19 | 63457951 | 63458001 |
| chr19 | 63457808 | 63457858 |
| chr1 | 32779858 | 32779908 |
| chr1 | 89131112 | 89131162 |
| chr1 | 89131200 | 89131250 |
| chr1 | 89131276 | 89131326 |
| chr1 | 141306950 | 141307000 |
| chr1 | 228382395 | 228382445 |
| chr21 | 14152879 | 14152929 |
| chr21 | 14191313 | 14191363 |
| chr21 | 43283299 | 43283349 |
| chr22 | 23979345 | 23979395 |
| chr22 | 28153185 | 28153235 |
| chrX | 85731204 | 85731254 |
| chrX | 85731111 | 85731161 |
| chrY | 7402638 | 7402688 |
| chrY | 9553205 | 9553255 |
| chrY | 13599141 | 13599191 |
| chr2 | 35376023 | 35376073 |
| chr2 | 97949754 | 97949804 |
| chr2 | 97950249 | 97950299 |
| chr2 | 97950616 | 97950666 |
| chr2 | 111541407 | 111541457 |
| chr2 | 113704633 | 113704683 |
| chr2 | 232965021 | 232965071 |
| chr3 | 47844896 | 47844946 |
| chr3 | 75657517 | 75657567 |
| chr3 | 129716285 | 129716335 |
| chr3 | 180719847 | 180719897 |
| chr3 | 187936483 | 187936533 |
| chr5 | 79491046 | 79491096 |
| chr5 | 179149670 | 179149720 |
| chr6 | 2935949 | 2935999 |
| chr6 | 68885928 | 68885978 |
| chr11 | 107242740 | 107242790 |
| chr7 | 101534025 | 101534075 |
| chr7 | 133649977 | 133650027 |
| chr8 | 30009411 | 30009461 |
| chr8 | 98747519 | 98747569 |
| chr9 | 86396222 | 86396272 |
| chr9 | 86396456 | 86396506 |
| chr9 | 123333858 | 123333908 |
| chr9 | 125606080 | 125606130 |
| chr9 | 135241528 | 135241578 |
| chr12 | 9452497 | 9452547 |
| chr12 | 18240111 | 18240161 |
| chr13 | 19333225 | 19333275 |
| chr10 | 38228378 | 38228428 |
| chr10 | 54059815 | 54059865 |
| chr10 | 54059971 | 54060021 |
| chr10 | 54060368 | 54060418 |
| chr14 | 101831459 | 101831509 |
| chr14 | 104634957 | 104635007 |
| chr14 | 104963949 | 104963999 |
| chr15 | 43480475 | 43480525 |
| chr16 | 10322351 | 10322401 |
| chr16 | 35631024 | 35631074 |
| chr16 | 89422025 | 89422075 |

Table 15BN. Repeat Category LTR6B

| | | |
|---|---|---|
| chr19 | 8173809 | 8173859 |
| chr19 | 20229475 | 20229525 |
| chr19 | 41513125 | 41513175 |
| chr19 | 58969545 | 58969595 |
| chr19 | 63033640 | 63033690 |
| chr1 | 69148146 | 69148196 |
| chr1 | 99888558 | 99888608 |
| chr20 | 46514718 | 46514768 |
| chr21 | 40869833 | 40869883 |
| chr21 | 42020284 | 42020334 |
| chr21 | 42020433 | 42020483 |
| chr21 | 42391390 | 42391440 |
| chr21 | 42391462 | 42391512 |
| chrX | 2404210 | 2404260 |
| chrX | 101411671 | 101411721 |
| chrY | 7023532 | 7023582 |
| chr2 | 47899899 | 47899949 |
| chr2 | 124544225 | 124544275 |
| chr2 | 202193112 | 202193162 |
| chr2 | 212659410 | 212659460 |
| chr3 | 75476646 | 75476696 |
| chr3 | 126872281 | 126872331 |
| chr3 | 131223375 | 131223425 |
| chr3 | 147450182 | 147450232 |
| chr3 | 165380052 | 165380102 |
| chr3 | 188049743 | 188049793 |
| chr4 | 9352921 | 9352971 |
| chr11 | 67424875 | 67424925 |
| chr11 | 67431729 | 67431779 |
| chr4 | 177090399 | 177090449 |
| chr7 | 6779170 | 6779220 |
| chr7 | 138190863 | 138190913 |
| chr8 | 6992924 | 6992974 |
| chr8 | 17040286 | 17040336 |
| chr8 | 17040150 | 17040200 |
| chr8 | 42705163 | 42705213 |
| chr8 | 138002465 | 138002515 |
| chr8 | 145925947 | 145925997 |
| chr12 | 22745308 | 22745358 |
| chr13 | 67835085 | 67835135 |
| chr14 | 100521088 | 100521138 |
| chr15 | 86811697 | 86811747 |
| chr15 | 86819392 | 86819442 |
| chr16 | 87599119 | 87599169 |

Table 15BO. Repeat Category LTR7

| | | |
|---|---|---|
| chr17 | 41458920 | 41458970 |
| chr17 | 53570257 | 53570307 |
| chr17 | 54019740 | 54019790 |
| chr17 | 58109200 | 58109250 |
| chr17 | 63705550 | 63705600 |
| chr17 | 65970811 | 65970861 |
| chr17 | 75204469 | 75204519 |
| chr17 | 78931863 | 78931913 |
| chr17 | 79074224 | 79074274 |
| chr10 | 91725026 | 91725076 |
| chr10 | 92237746 | 92237796 |
| chr10 | 92769228 | 92769278 |
| chr18 | 11668056 | 11668106 |
| chr18 | 14117968 | 14118018 |
| chr18 | 19122011 | 19122061 |
| chr18 | 27658717 | 27658767 |
| chr18 | 28113604 | 28113654 |
| chr18 | 31048632 | 31048682 |
| chr18 | 31193724 | 31193774 |
| chr10 | 96653888 | 96653938 |
| chr10 | 96655229 | 96655279 |
| chr18 | 38574279 | 38574329 |
| chr18 | 48585619 | 48585669 |
| chr18 | 52232209 | 52232259 |
| chr18 | 60497901 | 60497951 |
| chr18 | 69141295 | 69141345 |
| chr18 | 73753954 | 73754004 |
| chr10 | 98986394 | 98986444 |
| chr18 | 74397382 | 74397432 |
| chr19 | 320411 | 320461 |
| chr10 | 5472459 | 5472509 |
| chr19 | 2163171 | 2163221 |
| chr19 | 2852265 | 2852315 |
| chr19 | 5797840 | 5797890 |
| chr19 | 5808683 | 5808733 |
| chr19 | 10005395 | 10005445 |
| chr19 | 12341118 | 12341168 |
| chr19 | 12342474 | 12342524 |
| chr19 | 12543359 | 12543409 |
| chr19 | 15801176 | 15801226 |
| chr19 | 15801049 | 15801099 |
| chr10 | 103244268 | 103244318 |
| chr19 | 17869895 | 17869945 |
| chr10 | 103617621 | 103617671 |
| chr19 | 20003389 | 20003439 |
| chr19 | 20238122 | 20238172 |
| chr19 | 21291041 | 21291091 |
| chr19 | 21841739 | 21841789 |
| chr19 | 22408793 | 22408843 |
| chr19 | 22420613 | 22420663 |
| chr19 | 23000189 | 23000239 |
| chr19 | 23000636 | 23000686 |
| chr19 | 23554551 | 23554601 |
| chr19 | 33618498 | 33618548 |
| chr19 | 33618447 | 33618497 |
| chr19 | 39696735 | 39696785 |
| chr19 | 40787816 | 40787866 |
| chr19 | 41442157 | 41442207 |
| chr19 | 42019143 | 42019193 |
| chr19 | 42617725 | 42617775 |
| chr19 | 44723633 | 44723683 |
| chr19 | 45157085 | 45157135 |
| chr10 | 107447434 | 107447484 |
| chr19 | 51473497 | 51473547 |
| chr10 | 110023910 | 110023960 |
| chr10 | 110987213 | 110987263 |
| chr19 | 52244705 | 52244755 |
| chr19 | 52587809 | 52587859 |
| chr19 | 52593911 | 52593961 |
| chr19 | 52594048 | 52594098 |
| chr19 | 57373376 | 57373426 |
| chr19 | 57823038 | 57823088 |
| chr19 | 59032669 | 59032719 |
| chr19 | 59441078 | 59441128 |
| chr19 | 59441135 | 59441185 |
| chr19 | 59596809 | 59596859 |
| chr19 | 59748223 | 59748273 |
| chr19 | 60215651 | 60215701 |
| chr19 | 60332108 | 60332158 |
| chr19 | 62966742 | 62966792 |
| chr1 | 4775024 | 4775074 |
| chr10 | 117904962 | 117905012 |
| chr1 | 12700193 | 12700243 |
| chr1 | 13249804 | 13249854 |
| chr1 | 16604266 | 16604316 |
| chr1 | 20408931 | 20408981 |
| chr1 | 23170475 | 23170525 |
| chr1 | 25842410 | 25842460 |
| chr1 | 40838589 | 40838639 |
| chr1 | 41704211 | 41704261 |
| chr1 | 42075344 | 42075394 |
| chr10 | 122004355 | 122004405 |
| chr1 | 48050121 | 48050171 |
| chr1 | 53285438 | 53285488 |
| chr10 | 123345115 | 123345165 |
| chr1 | 54858123 | 54858173 |
| chr1 | 57579138 | 57579188 |
| chr1 | 58539268 | 58539318 |
| chr1 | 64191436 | 64191486 |
| chr1 | 64973506 | 64973556 |
| chr1 | 64981409 | 64981459 |
| chr1 | 65478775 | 65478825 |
| chr1 | 65483613 | 65483663 |
| chr1 | 68221505 | 68221555 |
| chr1 | 68227690 | 68227740 |
| chr1 | 72821073 | 72821123 |
| chr1 | 72829291 | 72829341 |
| chr1 | 73207164 | 73207214 |
| chr1 | 74230932 | 74230982 |
| chr1 | 81961181 | 81961231 |
| chr1 | 83947197 | 83947247 |
| chr10 | 6675476 | 6675526 |
| chr1 | 91510808 | 91510858 |
| chr1 | 94595290 | 94595340 |
| chr10 | 6848897 | 6848947 |
| chr1 | 108362484 | 108362534 |
| chr1 | 112661402 | 112661452 |
| chr1 | 113878663 | 113878713 |
| chr1 | 116751098 | 116751148 |
| chr1 | 116924916 | 116924966 |
| chr1 | 144219839 | 144219889 |
| chr1 | 145434524 | 145434574 |
| chr1 | 145504231 | 145504281 |
| chr1 | 146555801 | 146555851 |
| chr1 | 146586234 | 146586284 |
| chr1 | 146729526 | 146729576 |
| chr1 | 149094901 | 149094951 |
| chr1 | 170333388 | 170333438 |
| chr1 | 178129472 | 178129522 |
| chr1 | 180821684 | 180821734 |
| chr1 | 183156718 | 183156768 |
| chr1 | 183162799 | 183162849 |
| chr1 | 184973575 | 184973625 |
| chr1 | 184979006 | 184979056 |
| chr1 | 190237348 | 190237398 |
| chr1 | 193472613 | 193472663 |
| chr1 | 200259625 | 200259675 |
| chr1 | 207440302 | 207440352 |
| chr1 | 210138031 | 210138081 |
| chr1 | 210144500 | 210144550 |
| chr1 | 212640042 | 212640092 |
| chr1 | 212645171 | 212645221 |
| chr1 | 213237948 | 213237998 |
| chr1 | 213243798 | 213243848 |
| chr1 | 213382532 | 213382582 |
| chr1 | 219208696 | 219208746 |
| chr1 | 220253922 | 220253972 |
| chr1 | 222002013 | 222002063 |
| chr1 | 224803468 | 224803518 |
| chr1 | 226277275 | 226277325 |
| chr1 | 226793704 | 226793754 |
| chr1 | 227900599 | 227900649 |
| chr1 | 229006381 | 229006431 |
| chr1 | 229288686 | 229288736 |
| chr1 | 229294903 | 229294953 |
| chr1 | 234142406 | 234142456 |
| chr1 | 234693440 | 234693490 |
| chr1 | 235337658 | 235337708 |
| chr1 | 235480380 | 235480430 |
| chr1 | 235584318 | 235584368 |
| chr1 | 236013819 | 236013869 |
| chr1 | 237968428 | 237968478 |
| chr1 | 241460664 | 241460714 |
| chr1 | 243528719 | 243528769 |
| chr1 | 243795101 | 243795151 |
| chr1 | 243796303 | 243796353 |
| chr1 | 244189592 | 244189642 |
| chr1 | 244470329 | 244470379 |
| chr20 | 742482 | 742532 |
| chr20 | 4635833 | 4635883 |
| chr20 | 12321818 | 12321868 |
| chr20 | 12735264 | 12735314 |
| chr20 | 13338506 | 13338556 |
| chr20 | 13876308 | 13876358 |
| chr20 | 18492481 | 18492531 |
| chr20 | 18498196 | 18498246 |
| chr20 | 19727641 | 19727691 |
| chr20 | 21646042 | 21646092 |
| chr20 | 32272146 | 32272196 |
| chr20 | 36304577 | 36304627 |
| chr20 | 39407139 | 39407189 |
| chr20 | 39413065 | 39413115 |
| chr20 | 42696340 | 42696390 |
| chr20 | 43839348 | 43839398 |
| chr20 | 53701485 | 53701535 |
| chr20 | 55854040 | 55854090 |
| chr20 | 59976389 | 59976439 |
| chr20 | 61136928 | 61136978 |
| chr21 | 14326082 | 14326132 |
| chr21 | 19415400 | 19415450 |
| chr21 | 21028477 | 21028527 |
| chr21 | 24121515 | 24121565 |
| chr21 | 24216753 | 24216803 |
| chr21 | 26522033 | 26522083 |
| chr21 | 30043571 | 30043621 |
| chr21 | 37884951 | 37885001 |
| chr21 | 40990842 | 40990892 |
| chr21 | 41246842 | 41246892 |
| chr21 | 41253213 | 41253263 |
| chr21 | 42101473 | 42101523 |
| chr21 | 43114939 | 43114989 |
| chr21 | 44618204 | 44618254 |
| chr21 | 44759565 | 44759615 |
| chr11 | 1314631 | 1314681 |
| chr22 | 18398743 | 18398793 |
| chr22 | 18658204 | 18658254 |
| chr22 | 19187029 | 19187079 |
| chr22 | 19187947 | 19187997 |
| chr22 | 23412040 | 23412090 |
| chr22 | 26462786 | 26462836 |
| chr22 | 32555208 | 32555258 |
| chr22 | 37678648 | 37678698 |
| chr22 | 37701491 | 37701541 |
| chr22 | 37769940 | 37769990 |
| chr22 | 41192174 | 41192224 |
| chr22 | 42291160 | 42291210 |
| chr22 | 43803544 | 43803594 |
| chr22 | 44286645 | 44286695 |
| chr22 | 46694833 | 46694883 |
| chrX | 3066239 | 3066289 |
| chrX | 3103984 | 3104034 |
| chrX | 4276859 | 4276909 |
| chrX | 5687488 | 5687538 |
| chrX | 12319325 | 12319375 |
| chr11 | 5669400 | 5669450 |
| chr11 | 6055258 | 6055308 |
| chr11 | 6061449 | 6061499 |
| chrX | 29409331 | 29409381 |
| chrX | 29483767 | 29483817 |
| chrX | 29834387 | 29834437 |
| chrX | 30757139 | 30757189 |
| chrX | 30762825 | 30762875 |
| chrX | 32483243 | 32483293 |
| chr11 | 6351533 | 6351583 |
| chrX | 39723070 | 39723120 |
| chrX | 41897661 | 41897711 |
| chrX | 46188728 | 46188778 |
| chrX | 47494834 | 47494884 |
| chrX | 69351664 | 69351714 |
| chrX | 72238335 | 72238385 |
| chrX | 85714508 | 85714558 |
| chrX | 85715676 | 85715726 |
| chrX | 87611397 | 87611447 |
| chrX | 87613151 | 87613201 |
| chrX | 87783484 | 87783534 |
| chrX | 90127018 | 90127068 |
| chrX | 92725642 | 92725692 |
| chrX | 92731936 | 92731986 |
| chrX | 96316297 | 96316347 |
| chrX | 98603436 | 98603486 |
| chrX | 100760285 | 100760335 |
| chrX | 101455806 | 101455856 |
| chrX | 107836776 | 107836826 |
| chrX | 109550892 | 109550942 |
| chrX | 111187735 | 111187785 |
| chrX | 112054385 | 112054435 |
| chrX | 112465245 | 112465295 |
| chrX | 115172922 | 115172972 |
| chrX | 117205817 | 117205867 |
| chrX | 120059532 | 120059582 |
| chrX | 121608817 | 121608867 |
| chrX | 123228928 | 123228978 |
| chrX | 123333255 | 123333305 |
| chrX | 132785064 | 132785114 |
| chrX | 137701174 | 137701224 |
| chrX | 140204738 | 140204788 |
| chrX | 140212967 | 140213017 |
| chrX | 143475247 | 143475297 |
| chrY | 2753777 | 2753827 |
| chrY | 2756111 | 2756161 |
| chrY | 4900878 | 4900928 |
| chr11 | 14510814 | 14510864 |
| chrY | 19702078 | 19702128 |
| chr2 | 20468938 | 20468988 |
| chr2 | 22051998 | 22052048 |
| chr2 | 797109 | 797159 |
| chr2 | 8040816 | 8040866 |
| chr2 | 16957702 | 16957752 |
| chr2 | 16963579 | 16963629 |
| chr2 | 17990089 | 17990139 |
| chr2 | 27941470 | 27941520 |
| chr2 | 35641735 | 35641785 |
| chr2 | 37025262 | 37025312 |
| chr2 | 38590343 | 38590393 |
| chr2 | 44226357 | 44226407 |
| chr2 | 45241595 | 45241645 |
| chr11 | 20553729 | 20553779 |
| chr2 | 47899899 | 47899949 |
| chr2 | 58315050 | 58315100 |
| chr2 | 58320785 | 58320835 |
| chr2 | 60619064 | 60619114 |
| chr11 | 23169493 | 23169543 |
| chr2 | 64459301 | 64459351 |
| chr2 | 69650337 | 69650387 |
| chr11 | 25472316 | 25472366 |
| chr2 | 70822185 | 70822235 |
| chr2 | 70828102 | 70828152 |
| chr2 | 71287939 | 71287989 |
| chr2 | 71292495 | 71292545 |
| chr2 | 75415360 | 75415410 |
| chr2 | 76775249 | 76775299 |
| chr2 | 77290465 | 77290515 |
| chr2 | 78102254 | 78102304 |
| chr2 | 78965979 | 78966029 |
| chr2 | 81081809 | 81081859 |
| chr11 | 27620810 | 27620860 |
| chr2 | 85266720 | 85266770 |
| chr2 | 99848100 | 99848150 |
| chr2 | 101416235 | 101416285 |
| chr11 | 32129805 | 32129855 |
| chr11 | 32135280 | 32135330 |
| chr2 | 110712182 | 110712232 |
| chr2 | 111599503 | 111599553 |
| chr2 | 115643625 | 115643675 |
| chr2 | 138657080 | 138657130 |
| chr2 | 138790890 | 138790940 |
| chr2 | 154593483 | 154593533 |
| chr2 | 166766568 | 166766618 |
| chr2 | 173232361 | 173232411 |
| chr11 | 37760541 | 37760591 |
| chr2 | 184111025 | 184111075 |
| chr2 | 184128127 | 184128177 |
| chr2 | 184597144 | 184597194 |
| chr2 | 188633405 | 188633455 |
| chr2 | 188743059 | 188743109 |
| chr2 | 189100605 | 189100655 |
| chr2 | 189235924 | 189235974 |
| chr2 | 189241624 | 189241674 |
| chr2 | 193197623 | 193197673 |
| chr2 | 200319991 | 200320041 |
| chr11 | 39720435 | 39720485 |
| chr2 | 207049639 | 207049689 |
| chr2 | 208084452 | 208084502 |
| chr2 | 209961245 | 209961295 |
| chr2 | 210151784 | 210151834 |
| chr2 | 210366552 | 210366602 |
| chr2 | 210372381 | 210372431 |
| chr11 | 42111217 | 42111267 |
| chr2 | 216995419 | 216995469 |
| chr11 | 43974323 | 43974373 |
| chr2 | 223915799 | 223915849 |
| chr2 | 232637365 | 232637415 |
| chr2 | 236326913 | 236326963 |
| chr2 | 238808029 | 238808079 |
| chr2 | 239420221 | 239420271 |
| chr3 | 5165886 | 5165936 |
| chr3 | 6738091 | 6738141 |
| chr3 | 10747574 | 10747624 |
| chr3 | 11287847 | 11287897 |
| chr3 | 14288629 | 14288679 |
| chr3 | 16760664 | 16760714 |
| chr3 | 27616047 | 27616097 |
| chr3 | 32473898 | 32473948 |
| chr3 | 32479540 | 32479590 |
| chr3 | 32809601 | 32809651 |
| chr3 | 38085537 | 38085587 |
| chr3 | 41966905 | 41966955 |
| chr3 | 45315474 | 45315524 |
| chr3 | 46246893 | 46246943 |
| chr3 | 46246379 | 46246429 |
| chr3 | 46558940 | 46558990 |
| chr3 | 48232390 | 48232440 |
| chr3 | 48726495 | 48726545 |
| chr3 | 51795554 | 51795604 |
| chr3 | 54625825 | 54625875 |
| chr3 | 54631560 | 54631610 |
| chr3 | 57295022 | 57295072 |
| chr3 | 61635405 | 61635455 |
| chr3 | 71894904 | 71894954 |
| chr3 | 75445268 | 75445318 |
| chr3 | 78559109 | 78559159 |
| chr3 | 95868837 | 95868887 |
| chr3 | 97249984 | 97250034 |
| chr3 | 99284120 | 99284170 |
| chr3 | 102658141 | 102658191 |
| chr3 | 106077332 | 106077382 |
| chr3 | 113458126 | 113458176 |
| chr3 | 113463226 | 113463276 |
| chr3 | 119773564 | 119773614 |
| chr3 | 120165128 | 120165178 |
| chr3 | 125797321 | 125797371 |
| chr3 | 125867722 | 125867772 |
| chr3 | 129874226 | 129874276 |
| chr3 | 129881265 | 129881315 |
| chr3 | 131104699 | 131104749 |
| chr3 | 134474740 | 134474790 |
| chr3 | 144112238 | 144112288 |
| chr3 | 146595058 | 146595108 |
| chr3 | 149452834 | 149452884 |
| chr3 | 149458833 | 149458883 |
| chr3 | 150235942 | 150235992 |
| chr3 | 153635679 | 153635729 |
| chr11 | 61587529 | 61587579 |
| chr3 | 154264772 | 154264822 |
| chr3 | 154271240 | 154271290 |
| chr3 | 154867467 | 154867517 |
| chr3 | 157083847 | 157083897 |
| chr3 | 157083481 | 157083531 |
| chr3 | 165386064 | 165386114 |
| chr3 | 171717038 | 171717088 |
| chr3 | 175270817 | 175270867 |
| chr3 | 177100576 | 177100626 |
| chr3 | 177365415 | 177365465 |
| chr3 | 178704449 | 178704499 |
| chr3 | 178709974 | 178710024 |
| chr3 | 180555561 | 180555611 |
| chr3 | 180564544 | 180564594 |
| chr3 | 183781376 | 183781426 |
| chr3 | 187702397 | 187702447 |
| chr3 | 188224308 | 188224358 |
| chr3 | 192934349 | 192934399 |
| chr3 | 193109909 | 193109959 |
| chr3 | 193115542 | 193115592 |
| chr3 | 196763750 | 196763800 |
| chr3 | 196763450 | 196763500 |
| chr4 | 45068 | 45118 |
| chr4 | 112371 | 112421 |
| chr4 | 283783 | 283833 |
| chr4 | 324789 | 324839 |
| chr4 | 5115780 | 5115830 |
| chr4 | 5521949 | 5521999 |
| chr4 | 6596559 | 6596609 |
| chr4 | 13928461 | 13928511 |
| chr4 | 16727142 | 16727192 |
| chr4 | 16756591 | 16756641 |
| chr4 | 18769231 | 18769281 |
| chr4 | 23475191 | 23475241 |
| chr4 | 24080699 | 24080749 |
| chr4 | 24253307 | 24253357 |
| chr4 | 24259449 | 24259499 |
| chr4 | 30017200 | 30017250 |
| chr4 | 33948271 | 33948321 |
| chr4 | 47685906 | 47685956 |
| chr4 | 54802023 | 54802073 |
| chr4 | 54807245 | 54807295 |
| chr4 | 62639104 | 62639154 |
| chr4 | 63224036 | 63224086 |
| chr4 | 64854425 | 64854475 |
| chr4 | 77836731 | 77836781 |
| chr4 | 80589844 | 80589894 |
| chr4 | 80933231 | 80933281 |
| chr4 | 80939356 | 80939406 |
| chr4 | 82800313 | 82800363 |
| chr4 | 83669442 | 83669492 |
| chr4 | 85016892 | 85016942 |
| chr4 | 85230161 | 85230211 |
| chr4 | 89315394 | 89315444 |
| chr4 | 89532039 | 89532089 |
| chr4 | 91350011 | 91350061 |
| chr4 | 93405249 | 93405299 |
| chr4 | 93410676 | 93410726 |
| chr4 | 93650984 | 93651034 |
| chr4 | 93818464 | 93818514 |
| chr4 | 93824180 | 93824230 |
| chr4 | 95546322 | 95546372 |
| chr4 | 95678094 | 95678144 |
| chr4 | 98519383 | 98519433 |
| chr4 | 104939518 | 104939568 |
| chr11 | 66119805 | 66119855 |
| chr4 | 110094867 | 110094917 |
| chr4 | 118424332 | 118424382 |
| chr4 | 121379625 | 121379675 |
| chr4 | 124392873 | 124392923 |
| chr4 | 127530500 | 127530550 |
| chr4 | 130026112 | 130026162 |
| chr4 | 132147347 | 132147397 |
| chr4 | 133498126 | 133498176 |
| chr4 | 133504043 | 133504093 |
| chr4 | 134648395 | 134648445 |
| chr4 | 136553172 | 136553222 |
| chr4 | 139611876 | 139611926 |
| chr4 | 140426584 | 140426634 |
| chr4 | 140656729 | 140656779 |
| chr4 | 147202267 | 147202317 |
| chr4 | 147337526 | 147337576 |
| chr4 | 154245912 | 154245962 |
| chr4 | 157608004 | 157608054 |
| chr4 | 158099132 | 158099182 |
| chr4 | 158104885 | 158104935 |
| chr4 | 159095522 | 159095572 |
| chr4 | 160923873 | 160923923 |
| chr4 | 161424822 | 161424872 |
| chr11 | 67192878 | 67192928 |
| chr11 | 67193429 | 67193479 |
| chr4 | 164591811 | 164591861 |
| chr4 | 164596397 | 164596447 |
| chr4 | 168332962 | 168333012 |
| chr4 | 169057998 | 169058048 |
| chr4 | 169635601 | 169635651 |
| chr4 | 176105575 | 176105625 |
| chr4 | 176137194 | 176137244 |
| chr4 | 177911249 | 177911299 |
| chr4 | 179124771 | 179124821 |
| chr4 | 180787874 | 180787924 |
| chr4 | 181021088 | 181021138 |
| chr4 | 183567576 | 183567626 |
| chr4 | 184436135 | 184436185 |
| chr4 | 186284161 | 186284211 |
| chr4 | 187660968 | 187661018 |
| chr4 | 187859292 | 187859342 |
| chr5 | 912356 | 912406 |
| chr5 | 5743960 | 5744010 |
| chr5 | 7316324 | 7316374 |
| chr5 | 7322196 | 7322246 |
| chr5 | 8467325 | 8467375 |
| chr5 | 11643289 | 11643339 |
| chr5 | 12542807 | 12542857 |
| chr5 | 12547376 | 12547426 |
| chr5 | 12721243 | 12721293 |
| chr5 | 17119212 | 17119262 |
| chr5 | 18580782 | 18580832 |
| chr5 | 18585098 | 18585148 |
| chr5 | 18589610 | 18589660 |
| chr5 | 18720260 | 18720310 |
| chr5 | 20382016 | 20382066 |
| chr5 | 25491202 | 25491252 |
| chr5 | 28975751 | 28975801 |
| chr5 | 66709229 | 66709279 |
| chr5 | 75743206 | 75743256 |
| chr11 | 70775900 | 70775950 |
| chr5 | 79563182 | 79563232 |
| chr5 | 79565564 | 79565614 |
| chr11 | 70943339 | 70943389 |
| chr5 | 84084130 | 84084180 |
| chr5 | 84244676 | 84244726 |
| chr5 | 85027506 | 85027556 |
| chr5 | 91124264 | 91124314 |
| chr5 | 100568126 | 100568176 |
| chr5 | 103443513 | 103443563 |
| chr5 | 106396051 | 106396101 |
| chr5 | 107966923 | 107966973 |
| chr5 | 107979518 | 107979568 |
| chr5 | 116027447 | 116027497 |
| chr5 | 116234548 | 116234598 |
| chr5 | 116357235 | 116357285 |
| chr5 | 116464191 | 116464241 |
| chr5 | 118364360 | 118364410 |
| chr5 | 120109423 | 120109473 |
| chr5 | 121886011 | 121886061 |
| chr5 | 121890851 | 121890901 |
| chr5 | 122036268 | 122036318 |
| chr5 | 126085557 | 126085607 |
| chr5 | 126251216 | 126251266 |
| chr5 | 126257770 | 126257820 |
| chr5 | 128082581 | 128082631 |
| chr5 | 130984055 | 130984105 |
| chr5 | 133274584 | 133274634 |
| chr5 | 135149379 | 135149429 |
| chr5 | 152863481 | 152863531 |
| chr5 | 162741949 | 162741999 |
| chr5 | 166705983 | 166706033 |
| chr5 | 170169927 | 170169977 |
| chr5 | 170416099 | 170416149 |
| chr5 | 170777459 | 170777509 |
| chr5 | 178730265 | 178730315 |
| chr5 | 179957273 | 179957323 |
| chr6 | 5973003 | 5973053 |
| chr11 | 77517974 | 77518024 |
| chr6 | 14411081 | 14411131 |
| chr6 | 16367130 | 16367180 |
| chr6 | 21470246 | 21470296 |
| chr6 | 21475468 | 21475518 |
| chr6 | 26136987 | 26137037 |
| chr6 | 28852995 | 28853045 |
| chr6 | 29057048 | 29057098 |
| chr6 | 31843729 | 31843779 |
| chr6 | 33895038 | 33895088 |
| chr6 | 38936351 | 38936401 |
| chr6 | 39150625 | 39150675 |
| chr6 | 43385251 | 43385301 |
| chr6 | 43406107 | 43406157 |
| chr6 | 44838237 | 44838287 |
| chr11 | 87471184 | 87471234 |
| chr6 | 63499024 | 63499074 |
| chr6 | 66554993 | 66555043 |
| chr6 | 68579259 | 68579309 |
| chr6 | 68594377 | 68594427 |
| chr6 | 68594473 | 68594523 |
| chr6 | 77266307 | 77266357 |
| chr6 | 78363667 | 78363717 |
| chr6 | 88567143 | 88567193 |
| chr6 | 91380742 | 91380792 |
| chr6 | 94536117 | 94536167 |
| chr6 | 94541254 | 94541304 |
| chr6 | 95259116 | 95259166 |
| chr6 | 95264979 | 95265029 |
| chr6 | 110222353 | 110222403 |
| chr11 | 94730706 | 94730756 |
| chr6 | 114794998 | 114795048 |
| chr6 | 120764033 | 120764083 |
| chr6 | 120769836 | 120769886 |
| chr6 | 123147437 | 123147487 |
| chr6 | 123889807 | 123889857 |
| chr6 | 125199519 | 125199569 |
| chr6 | 126009568 | 126009618 |
| chr6 | 129048307 | 129048357 |
| chr6 | 131013775 | 131013825 |
| chr6 | 131597688 | 131597738 |
| chr6 | 131603056 | 131603106 |
| chr6 | 131640988 | 131641038 |
| chr6 | 131810907 | 131810957 |
| chr6 | 137361814 | 137361864 |
| chr6 | 137368815 | 137368865 |
| chr6 | 142323680 | 142323730 |
| chr6 | 142950986 | 142951036 |
| chr6 | 145230567 | 145230617 |
| chr6 | 145410544 | 145410594 |
| chr6 | 154274812 | 154274862 |
| chr6 | 157328521 | 157328571 |
| chr6 | 158030869 | 158030919 |
| chr6 | 159210674 | 159210724 |
| chr6 | 160564770 | 160564820 |
| chr6 | 165380628 | 165380678 |
| chr6 | 165388524 | 165388574 |
| chr11 | 107287554 | 107287604 |
| chr7 | 944810 | 944860 |
| chr7 | 3857917 | 3857967 |
| chr7 | 6745920 | 6745970 |
| chr7 | 9142563 | 9142613 |
| chr7 | 19905094 | 19905144 |
| chr7 | 20330595 | 20330645 |
| chr7 | 24178459 | 24178509 |
| chr7 | 24183918 | 24183968 |
| chr7 | 25811936 | 25811986 |
| chr7 | 26053306 | 26053356 |
| chr7 | 32297009 | 32297059 |
| chr7 | 32620141 | 32620191 |
| chr7 | 32847359 | 32847409 |
| chr7 | 34081861 | 34081911 |
| chr7 | 42650927 | 42650977 |
| chr7 | 42656407 | 42656457 |
| chr7 | 42983531 | 42983581 |
| chr7 | 50914770 | 50914820 |
| chr7 | 64731010 | 64731060 |
| chr7 | 67266410 | 67266460 |
| chr7 | 67415464 | 67415514 |
| chr7 | 70051472 | 70051522 |
| chr7 | 75020685 | 75020735 |
| chr7 | 76581874 | 76581924 |
| chr7 | 76587031 | 76587081 |
| chr7 | 82706787 | 82706837 |
| chr7 | 99017947 | 99017997 |
| chr7 | 100136893 | 100136943 |
| chr7 | 105768288 | 105768338 |
| chr7 | 111051718 | 111051768 |
| chr7 | 112434981 | 112435031 |
| chr7 | 112439865 | 112439915 |
| chr7 | 119977531 | 119977581 |
| chr7 | 124459177 | 124459227 |
| chr7 | 125104244 | 125104294 |
| chr7 | 128727639 | 128727689 |
| chr7 | 137429128 | 137429178 |
| chr7 | 138192994 | 138193044 |
| chr7 | 138560808 | 138560858 |
| chr7 | 146698049 | 146698099 |
| chr7 | 151059258 | 151059308 |
| chr10 | 17664563 | 17664613 |
| chr8 | 1737344 | 1737394 |
| chr11 | 121045678 | 121045728 |
| chr8 | 2156882 | 2156932 |
| chr8 | 5281562 | 5281612 |
| chr8 | 7238340 | 7238390 |
| chr11 | 121963934 | 121963984 |
| chr8 | 8953796 | 8953846 |
| chr8 | 9698422 | 9698472 |
| chr11 | 123206002 | 123206052 |
| chr11 | 123273850 | 123273900 |
| chr11 | 123279090 | 123279140 |
| chr8 | 27752817 | 27752867 |
| chr8 | 30008238 | 30008288 |
| chr8 | 33541974 | 33542024 |
| chr8 | 34775340 | 34775390 |
| chr8 | 42997700 | 42997750 |
| chr8 | 58836871 | 58836921 |
| chr8 | 60838329 | 60838379 |
| chr8 | 68682129 | 68682179 |
| chr8 | 72642702 | 72642752 |
| chr8 | 76179650 | 76179700 |
| chr8 | 80353864 | 80353914 |
| chr8 | 82881597 | 82881647 |
| chr8 | 84472759 | 84472809 |
| chr8 | 89711218 | 89711268 |
| chr8 | 93384807 | 93384857 |
| chr8 | 98143793 | 98143843 |
| chr8 | 98169472 | 98169522 |
| chr8 | 98175453 | 98175503 |
| chr8 | 98796663 | 98796713 |
| chr8 | 99559150 | 99559200 |
| chr8 | 100912384 | 100912434 |
| chr8 | 101464852 | 101464902 |
| chr8 | 101877391 | 101877441 |
| chr8 | 104438989 | 104439039 |
| chr8 | 109278996 | 109279046 |
| chr8 | 114484292 | 114484342 |
| chr8 | 115210934 | 115210984 |
| chr8 | 116783713 | 116783763 |
| chr8 | 116788284 | 116788334 |
| chr8 | 122987406 | 122987456 |
| chr8 | 124448649 | 124448699 |
| chr8 | 125497285 | 125497335 |
| chr8 | 129586445 | 129586495 |
| chr8 | 129883261 | 129883311 |
| chr8 | 132715467 | 132715517 |
| chr8 | 136756471 | 136756521 |
| chr11 | 129977196 | 129977246 |
| chr11 | 130161291 | 130161341 |
| chr11 | 130166667 | 130166717 |
| chr8 | 144225651 | 144225701 |
| chr8 | 144225523 | 144225573 |
| chr8 | 144331505 | 144331555 |
| chr8 | 144362178 | 144362228 |
| chr8 | 145922018 | 145922068 |
| chr9 | 2266226 | 2266276 |
| chr9 | 3740055 | 3740105 |
| chr9 | 3806137 | 3806187 |
| chr9 | 3891195 | 3891245 |
| chr9 | 3891098 | 3891148 |
| chr9 | 6706607 | 6706657 |
| chr9 | 6707491 | 6707541 |
| chr9 | 10880158 | 10880208 |
| chr9 | 11092415 | 11092465 |
| chr9 | 12431051 | 12431101 |
| chr9 | 12944109 | 12944159 |
| chr9 | 21477966 | 21478016 |
| chr9 | 21481096 | 21481146 |
| chr9 | 25278328 | 25278378 |
| chr9 | 31316087 | 31316137 |
| chr9 | 33367328 | 33367378 |
| chr9 | 33476124 | 33476174 |
| chr9 | 35110654 | 35110704 |
| chr9 | 41232777 | 41232827 |
| chr9 | 67482073 | 67482123 |
| chr9 | 75026755 | 75026805 |
| chr9 | 80778301 | 80778351 |
| chr9 | 80961619 | 80961669 |
| chr9 | 83539045 | 83539095 |
| chr9 | 84661072 | 84661122 |
| chr9 | 85479862 | 85479912 |
| chr9 | 85480482 | 85480532 |
| chr10 | 20663720 | 20663770 |
| chr9 | 86404273 | 86404323 |
| chr9 | 88516171 | 88516221 |
| chr9 | 88522155 | 88522205 |
| chr9 | 90175920 | 90175970 |
| chr9 | 92648983 | 92649033 |
| chr9 | 94929155 | 94929205 |
| chr9 | 95356579 | 95356629 |
| chr9 | 96407215 | 96407265 |
| chr9 | 96417384 | 96417434 |
| chr9 | 96596118 | 96596168 |
| chr9 | 97811822 | 97811872 |
| chr9 | 98288046 | 98288096 |
| chr9 | 113611254 | 113611304 |
| chr9 | 113977435 | 113977485 |
| chr9 | 113982690 | 113982740 |
| chr9 | 116623922 | 116623972 |
| chr9 | 119935335 | 119935385 |
| chr9 | 120674529 | 120674579 |
| chr9 | 124212720 | 124212770 |
| chr9 | 124218772 | 124218822 |
| chr9 | 129703599 | 129703649 |
| chr12 | 3496740 | 3496790 |
| chr10 | 22044281 | 22044331 |
| chr12 | 9878250 | 9878300 |
| chr12 | 10376784 | 10376834 |
| chr12 | 12102633 | 12102683 |
| chr12 | 13420931 | 13420981 |
| chr12 | 17071615 | 17071665 |
| chr12 | 17514646 | 17514696 |
| chr12 | 18240111 | 18240161 |
| chr12 | 18248000 | 18248050 |
| chr12 | 20866800 | 20866850 |
| chr10 | 23749942 | 23749992 |
| chr12 | 29184416 | 29184466 |
| chr12 | 29319814 | 29319864 |
| chr12 | 31876768 | 31876818 |
| chr12 | 43130641 | 43130691 |
| chr10 | 26061688 | 26061738 |
| chr10 | 26068046 | 26068096 |
| chr12 | 53627988 | 53628038 |
| chr12 | 65178259 | 65178309 |
| chr12 | 66452379 | 66452429 |
| chr12 | 71383160 | 71383210 |
| chr12 | 71389001 | 71389051 |
| chr12 | 71498252 | 71498302 |
| chr12 | 71506337 | 71506387 |
| chr12 | 78436041 | 78436091 |
| chr12 | 78441858 | 78441908 |
| chr12 | 102118271 | 102118321 |
| chr12 | 107340789 | 107340839 |
| chr12 | 112913349 | 112913399 |
| chr12 | 119337103 | 119337153 |
| chr12 | 119345589 | 119345639 |
| chr12 | 119345823 | 119345873 |
| chr12 | 126857744 | 126857794 |
| chr12 | 130271912 | 130271962 |
| chr12 | 130574418 | 130574468 |
| chr12 | 131705337 | 131705387 |
| chr12 | 131772354 | 131772404 |
| chr12 | 131772964 | 131773014 |
| chr13 | 23392028 | 23392078 |
| chr13 | 24885234 | 24885284 |
| chr13 | 28973530 | 28973580 |
| chr13 | 31232180 | 31232230 |
| chr13 | 33171348 | 33171398 |
| chr13 | 33175567 | 33175617 |
| chr13 | 41431715 | 41431765 |
| chr10 | 38084503 | 38084553 |
| chr13 | 43171764 | 43171814 |
| chr13 | 44666918 | 44666968 |
| chr13 | 46428340 | 46428390 |
| chr13 | 47835474 | 47835524 |
| chr13 | 49541967 | 49542017 |
| chr13 | 49547155 | 49547205 |
| chr13 | 52505058 | 52505108 |
| chr13 | 53956347 | 53956397 |
| chr13 | 54027032 | 54027082 |
| chr13 | 54033072 | 54033122 |
| chr13 | 54418429 | 54418479 |
| chr13 | 59577703 | 59577753 |
| chr13 | 61585295 | 61585345 |
| chr13 | 64598227 | 64598277 |
| chr13 | 64604546 | 64604596 |
| chr13 | 67569632 | 67569682 |
| chr13 | 76305325 | 76305375 |
| chr13 | 77646177 | 77646227 |
| chr13 | 77652117 | 77652167 |
| chr13 | 78562086 | 78562136 |
| chr13 | 80244312 | 80244362 |
| chr13 | 82285955 | 82286005 |
| chr13 | 83789886 | 83789936 |
| chr13 | 84115741 | 84115791 |
| chr13 | 89190230 | 89190280 |
| chr13 | 94351831 | 94351881 |
| chr13 | 96412027 | 96412077 |
| chr13 | 107615852 | 107615902 |
| chr13 | 107621559 | 107621609 |
| chr10 | 44535441 | 44535491 |
| chr10 | 44537559 | 44537609 |
| chr14 | 19273328 | 19273378 |
| chr14 | 22399362 | 22399412 |
| chr14 | 22399170 | 22399220 |
| chr14 | 23890038 | 23890088 |
| chr14 | 24989571 | 24989621 |
| chr14 | 29710654 | 29710704 |
| chr10 | 78646 | 78696 |
| chr14 | 36655915 | 36655965 |
| chr10 | 48887289 | 48887339 |
| chr14 | 44471582 | 44471632 |
| chr14 | 44477325 | 44477375 |
| chr14 | 45568223 | 45568273 |
| chr14 | 46727495 | 46727545 |
| chr14 | 48503541 | 48503591 |
| chr14 | 49425524 | 49425574 |
| chr14 | 59595494 | 59595544 |
| chr14 | 68758260 | 68758310 |
| chr14 | 69581419 | 69581469 |
| chr14 | 74042400 | 74042450 |
| chr10 | 53143958 | 53144008 |
| chr14 | 81682161 | 81682211 |
| chr14 | 84188743 | 84188793 |
| chr14 | 84194958 | 84195008 |
| chr14 | 87896544 | 87896594 |
| chr10 | 54596850 | 54596900 |
| chr10 | 54609192 | 54609242 |
| chr10 | 55276843 | 55276893 |
| chr14 | 92021451 | 92021501 |
| chr14 | 97449301 | 97449351 |
| chr14 | 99767723 | 99767773 |
| chr10 | 59198618 | 59198668 |
| chr10 | 59965264 | 59965314 |
| chr10 | 59971866 | 59971916 |
| chr15 | 25781138 | 25781188 |
| chr15 | 33263303 | 33263353 |
| chr15 | 44688314 | 44688364 |
| chr10 | 64536434 | 64536484 |
| chr15 | 51435015 | 51435065 |
| chr15 | 51440809 | 51440859 |
| chr15 | 53726040 | 53726090 |
| chr15 | 60305177 | 60305227 |
| chr15 | 61307738 | 61307788 |
| chr15 | 63527843 | 63527893 |
| chr15 | 63529569 | 63529619 |
| chr15 | 72257326 | 72257376 |
| chr15 | 73072346 | 73072396 |
| chr15 | 77886395 | 77886445 |
| chr15 | 77886175 | 77886225 |
| chr15 | 86103939 | 86103989 |
| chr15 | 86110020 | 86110070 |
| chr15 | 86820205 | 86820255 |
| chr15 | 88411340 | 88411390 |
| chr15 | 91052683 | 91052733 |
| chr15 | 95144767 | 95144817 |
| chr15 | 95150647 | 95150697 |
| chr15 | 98715686 | 98715736 |
| chr15 | 99348380 | 99348430 |
| chr16 | 65084 | 65134 |
| chr10 | 70158564 | 70158614 |
| chr16 | 3214500 | 3214550 |
| chr16 | 3304655 | 3304705 |
| chr16 | 7864320 | 7864370 |
| chr16 | 8894168 | 8894218 |
| chr16 | 8905961 | 8906011 |
| chr16 | 9731512 | 9731562 |
| chr16 | 10422510 | 10422560 |
| chr16 | 13412812 | 13412862 |
| chr16 | 13418459 | 13418509 |
| chr16 | 18968603 | 18968653 |
| chr16 | 19886301 | 19886351 |
| chr16 | 24469265 | 24469315 |
| chr16 | 25544758 | 25544808 |
| chr16 | 46982071 | 46982121 |
| chr10 | 73011958 | 73012008 |
| chr16 | 56483472 | 56483522 |
| chr16 | 59332285 | 59332335 |
| chr16 | 59893668 | 59893718 |
| chr16 | 65044363 | 65044413 |
| chr16 | 65088145 | 65088195 |
| chr16 | 71518632 | 71518682 |
| chr16 | 82924124 | 82924174 |
| chr16 | 84194441 | 84194491 |
| chr16 | 86093144 | 86093194 |
| chr16 | 86096590 | 86096640 |
| chr16 | 86556838 | 86556888 |
| chr16 | 89060522 | 89060572 |
| chr16 | 89234080 | 89234130 |
| chr17 | 5343692 | 5343742 |
| chr17 | 6841059 | 6841109 |
| chr17 | 7246205 | 7246255 |
| chr10 | 77922992 | 77923042 |
| chr17 | 8456399 | 8456449 |
| chr17 | 12080812 | 12080862 |
| chr17 | 17304534 | 17304584 |
| chr17 | 19958586 | 19958636 |
| chr17 | 32656351 | 32656401 |

Table 15BP. Repeat Category LTR7B

| | | |
|---|---|---|
| chr17 | 53570257 | 53570307 |
| chr17 | 65970811 | 65970861 |
| chr17 | 79074224 | 79074274 |
| chr10 | 92769228 | 92769278 |
| chr10 | 98986394 | 98986444 |
| chr19 | 320411 | 320461 |
| chr19 | 2852265 | 2852315 |
| chr19 | 5797840 | 5797890 |
| chr19 | 5808683 | 5808733 |
| chr19 | 41442157 | 41442207 |
| chr19 | 42617725 | 42617775 |
| chr10 | 110023910 | 110023960 |
| chr19 | 52244705 | 52244755 |
| chr19 | 59748223 | 59748273 |
| chr19 | 60332108 | 60332158 |
| chr1 | 13249804 | 13249854 |
| chr1 | 25842410 | 25842460 |
| chr1 | 40838589 | 40838639 |
| chr1 | 41704211 | 41704261 |
| chr1 | 57579138 | 57579188 |
| chr1 | 58539268 | 58539318 |
| chr1 | 64973506 | 64973556 |
| chr1 | 64981409 | 64981459 |
| chr1 | 91510808 | 91510858 |
| chr1 | 116751098 | 116751148 |
| chr1 | 200259625 | 200259675 |
| chr1 | 207440302 | 207440352 |
| chr1 | 234142406 | 234142456 |
| chr1 | 234693440 | 234693490 |
| chr1 | 235480380 | 235480430 |
| chr1 | 243528719 | 243528769 |
| chr1 | 243796303 | 243796353 |
| chr1 | 244189592 | 244189642 |
| chr20 | 4635833 | 4635883 |
| chr20 | 18492481 | 18492531 |
| chr20 | 18498196 | 18498246 |
| chr20 | 19727641 | 19727691 |
| chr20 | 55854040 | 55854090 |
| chr22 | 37701491 | 37701541 |
| chr22 | 41192174 | 41192224 |
| chr22 | 43803544 | 43803594 |
| chrX | 69351664 | 69351714 |
| chrX | 72238335 | 72238385 |
| chrX | 96316297 | 96316347 |
| chrX | 107836776 | 107836826 |
| chrX | 111187735 | 111187785 |
| chrX | 112054385 | 112054435 |
| chrX | 132785064 | 132785114 |
| chrX | 140212967 | 140213017 |
| chr11 | 14510814 | 14510864 |
| chr2 | 45241595 | 45241645 |
| chr2 | 99848100 | 99848150 |
| chr2 | 101416235 | 101416285 |
| chr2 | 138790890 | 138790940 |
| chr11 | 37760541 | 37760591 |
| chr2 | 184128127 | 184128177 |
| chr11 | 39720435 | 39720485 |
| chr2 | 223915799 | 223915849 |
| chr3 | 11287847 | 11287897 |
| chr3 | 27616047 | 27616097 |
| chr3 | 41966905 | 41966955 |
| chr3 | 99284120 | 99284170 |
| chr3 | 120165128 | 120165178 |
| chr3 | 129874226 | 129874276 |
| chr3 | 129881265 | 129881315 |
| chr3 | 154867467 | 154867517 |
| chr3 | 183781376 | 183781426 |
| chr4 | 30017200 | 30017250 |
| chr4 | 54802023 | 54802073 |
| chr4 | 54807245 | 54807295 |
| chr4 | 63224036 | 63224086 |
| chr4 | 77836731 | 77836781 |
| chr4 | 80589844 | 80589894 |
| chr4 | 83669442 | 83669492 |
| chr4 | 85016892 | 85016942 |
| chr4 | 164591811 | 164591861 |
| chr4 | 164596397 | 164596447 |
| chr5 | 11643289 | 11643339 |
| chr5 | 28975751 | 28975801 |
| chr5 | 130984055 | 130984105 |
| chr5 | 170416099 | 170416149 |
| chr5 | 170777459 | 170777509 |
| chr5 | 178730265 | 178730315 |
| chr6 | 29057048 | 29057098 |
| chr6 | 31843729 | 31843779 |
| chr6 | 33895038 | 33895088 |
| chr6 | 43385251 | 43385301 |
| chr11 | 87471184 | 87471234 |
| chr6 | 66554993 | 66555043 |
| chr6 | 68594377 | 68594427 |
| chr6 | 68594473 | 68594523 |
| chr6 | 78363667 | 78363717 |
| chr6 | 157328521 | 157328571 |
| chr6 | 159210674 | 159210724 |
| chr11 | 107287554 | 107287604 |
| chr7 | 944810 | 944860 |
| chr7 | 26053306 | 26053356 |
| chr7 | 70051472 | 70051522 |
| chr7 | 105768288 | 105768338 |
| chr7 | 137429128 | 137429178 |
| chr8 | 89711218 | 89711268 |
| chr8 | 98796663 | 98796713 |
| chr8 | 116788284 | 116788334 |
| chr8 | 122987406 | 122987456 |
| chr9 | 3891195 | 3891245 |
| chr9 | 3891098 | 3891148 |
| chr9 | 11092415 | 11092465 |
| chr9 | 33367328 | 33367378 |
| chr9 | 35110654 | 35110704 |
| chr9 | 86404273 | 86404323 |
| chr9 | 90175920 | 90175970 |
| chr9 | 92648983 | 92649033 |
| chr12 | 17071615 | 17071665 |
| chr10 | 23749942 | 23749992 |
| chr12 | 71389001 | 71389051 |
| chr12 | 107340789 | 107340839 |
| chr12 | 112913349 | 112913399 |
| chr12 | 131772354 | 131772404 |
| chr12 | 131772964 | 131773014 |
| chr13 | 23392028 | 23392078 |
| chr13 | 28973530 | 28973580 |
| chr13 | 41431715 | 41431765 |
| chr13 | 82285955 | 82286005 |
| chr13 | 96412027 | 96412077 |
| chr14 | 19273328 | 19273378 |
| chr14 | 74042400 | 74042450 |
| chr15 | 88411340 | 88411390 |
| chr16 | 8894168 | 8894218 |
| chr16 | 8905961 | 8906011 |
| chr16 | 18968603 | 18968653 |
| chr16 | 56483472 | 56483522 |
| chr16 | 84194441 | 84194491 |
| chr17 | 5343692 | 5343742 |
| chr17 | 6841059 | 6841109 |
| chr17 | 7246205 | 7246255 |
| chr17 | 8456399 | 8456449 |
| chr17 | 19958586 | 19958636 |

Table 15BQ. Repeat Category LTR7Y

| | | |
|---|---|---|
| chr10 | 91725026 | 91725076 |
| chr18 | 69141295 | 69141345 |
| chr19 | 5808683 | 5808733 |
| chr19 | 10005395 | 10005445 |
| chr19 | 15801176 | 15801226 |
| chr19 | 15801049 | 15801099 |
| chr19 | 21841739 | 21841789 |
| chr10 | 110987213 | 110987263 |
| chr1 | 12700193 | 12700243 |
| chr1 | 20408931 | 20408981 |
| chr1 | 81961181 | 81961231 |
| chr1 | 83947197 | 83947247 |
| chr1 | 94595290 | 94595340 |
| chr1 | 108362484 | 108362534 |
| chr1 | 184979006 | 184979056 |
| chr1 | 243795101 | 243795151 |
| chr20 | 12321818 | 12321868 |
| chr20 | 36304577 | 36304627 |
| chr20 | 39407139 | 39407189 |
| chr20 | 39413065 | 39413115 |
| chr22 | 19187029 | 19187079 |
| chr22 | 19187947 | 19187997 |
| chr22 | 26462786 | 26462836 |
| chr22 | 37678648 | 37678698 |
| chr22 | 37769940 | 37769990 |
| chrX | 3066239 | 3066289 |
| chrX | 115172922 | 115172972 |
| chrX | 117205817 | 117205867 |
| chr2 | 44226357 | 44226407 |
| chr11 | 20553729 | 20553779 |
| chr2 | 58315050 | 58315100 |
| chr2 | 58320785 | 58320835 |
| chr2 | 193197623 | 193197673 |
| chr11 | 42111217 | 42111267 |
| chr3 | 48726495 | 48726545 |
| chr3 | 125797321 | 125797371 |
| chr11 | 61587529 | 61587579 |
| chr3 | 157083847 | 157083897 |
| chr3 | 157083481 | 157083531 |
| chr3 | 188224308 | 188224358 |
| chr4 | 93405249 | 93405299 |
| chr4 | 93410676 | 93410726 |
| chr4 | 95678094 | 95678144 |
| chr4 | 104939518 | 104939568 |
| chr4 | 127530500 | 127530550 |
| chr4 | 139611876 | 139611926 |
| chr4 | 154245912 | 154245962 |
| chr4 | 169057998 | 169058048 |
| chr4 | 176105575 | 176105625 |
| chr4 | 184436135 | 184436185 |
| chr5 | 8467325 | 8467375 |
| chr5 | 12542807 | 12542857 |
| chr5 | 12547376 | 12547426 |
| chr5 | 79563182 | 79563232 |
| chr5 | 100568126 | 100568176 |
| chr5 | 121890851 | 121890901 |
| chr5 | 179957273 | 179957323 |
| chr11 | 77517974 | 77518024 |
| chr6 | 39150625 | 39150675 |
| chr11 | 94730706 | 94730756 |
| chr6 | 120764033 | 120764083 |
| chr6 | 125199519 | 125199569 |
| chr6 | 131810907 | 131810957 |
| chr6 | 145230567 | 145230617 |
| chr6 | 154274812 | 154274862 |
| chr7 | 25811936 | 25811986 |
| chr7 | 34081861 | 34081911 |
| chr7 | 112434981 | 112435031 |
| chr7 | 112439865 | 112439915 |
| chr8 | 80353864 | 80353914 |
| chr8 | 98143793 | 98143843 |
| chr8 | 99559150 | 99559200 |
| chr8 | 116783713 | 116783763 |
| chr8 | 136756471 | 136756521 |
| chr11 | 129977196 | 129977246 |
| chr9 | 21481096 | 21481146 |
| chr9 | 75026755 | 75026805 |
| chr9 | 80778301 | 80778351 |
| chr9 | 84661072 | 84661122 |
| chr12 | 17514646 | 17514696 |
| chr12 | 126857744 | 126857794 |
| chr13 | 33171348 | 33171398 |
| chr13 | 33175567 | 33175617 |
| chr13 | 89190230 | 89190280 |
| chr13 | 107615852 | 107615902 |
| chr13 | 107621559 | 107621609 |
| chr14 | 22399362 | 22399412 |
| chr14 | 22399170 | 22399220 |
| chr14 | 29710654 | 29710704 |
| chr14 | 81682161 | 81682211 |
| chr15 | 25781138 | 25781188 |
| chr15 | 33263303 | 33263353 |
| chr15 | 51435015 | 51435065 |
| chr15 | 51440809 | 51440859 |
| chr15 | 53726040 | 53726090 |
| chr15 | 91052683 | 91052733 |
| chr16 | 86093144 | 86093194 |
| chr16 | 86096590 | 86096640 |
| chr17 | 5343692 | 5343742 |
| chr10 | 77922992 | 77923042 |
| chr17 | 12080812 | 12080862 |
| chr17 | 32656351 | 32656401 |

Table 15BR. Repeat Category LTR8

| | | |
|---|---|---|
| chr17 | 38479086 | 38479136 |
| chr17 | 43389204 | 43389254 |
| chr17 | 47630768 | 47630818 |
| chr10 | 84099718 | 84099768 |
| chr17 | 66301781 | 66301831 |
| chr17 | 67940971 | 67941021 |
| chr17 | 71466373 | 71466423 |
| chr17 | 74576553 | 74576603 |
| chr17 | 76967249 | 76967299 |
| chr17 | 78838348 | 78838398 |
| chr17 | 80300784 | 80300834 |
| chr17 | 81389895 | 81389945 |
| chr10 | 93826357 | 93826407 |
| chr10 | 4863106 | 4863156 |
| chr18 | 53654288 | 53654338 |
| chr18 | 59994660 | 59994710 |
| chr18 | 66200360 | 66200410 |
| chr18 | 73429174 | 73429224 |
| chr19 | 211332 | 211382 |
| chr19 | 2109709 | 2109759 |
| chr19 | 2113827 | 2113877 |
| chr19 | 2417602 | 2417652 |
| chr19 | 2419597 | 2419647 |
| chr19 | 2904015 | 2904065 |
| chr19 | 7052532 | 7052582 |
| chr19 | 12008845 | 12008895 |
| chr19 | 12036997 | 12037047 |
| chr19 | 12038223 | 12038273 |
| chr19 | 12267023 | 12267073 |
| chr19 | 12268678 | 12268728 |
| chr19 | 12855135 | 12855185 |
| chr19 | 13636202 | 13636252 |
| chr19 | 14767399 | 14767449 |
| chr19 | 17387418 | 17387468 |
| chr19 | 18293716 | 18293766 |
| chr19 | 44047905 | 44047955 |
| chr19 | 44048422 | 44048472 |
| chr19 | 44048600 | 44048650 |
| chr19 | 44049077 | 44049127 |
| chr19 | 45375634 | 45375684 |
| chr10 | 105608127 | 105608177 |
| chr19 | 46997083 | 46997133 |
| chr19 | 49505457 | 49505507 |
| chr19 | 50773479 | 50773529 |
| chr19 | 50777525 | 50777575 |
| chr19 | 50778188 | 50778238 |
| chr19 | 53631010 | 53631060 |
| chr19 | 57098875 | 57098925 |
| chr19 | 60208510 | 60208560 |
| chr19 | 63022959 | 63023009 |
| chr19 | 63119545 | 63119595 |
| chr1 | 9281222 | 9281272 |
| chr1 | 10819727 | 10819777 |
| chr1 | 20317475 | 20317525 |
| chr1 | 24910672 | 24910722 |
| chr1 | 24911038 | 24911088 |
| chr1 | 31147815 | 31147865 |
| chr1 | 32604453 | 32604503 |
| chr10 | 6440911 | 6440961 |
| chr1 | 45193098 | 45193148 |
| chr1 | 62014127 | 62014177 |
| chr1 | 65134045 | 65134095 |
| chr1 | 75673274 | 75673324 |
| chr1 | 75673868 | 75673918 |
| chr1 | 75674105 | 75674155 |
| chr1 | 75674428 | 75674478 |
| chr1 | 89000532 | 89000582 |
| chr1 | 89000705 | 89000755 |
| chr1 | 89441007 | 89441057 |
| chr1 | 91159585 | 91159635 |
| chr1 | 99696143 | 99696193 |
| chr1 | 110109997 | 110110047 |
| chr1 | 158155769 | 158155819 |
| chr1 | 179647472 | 179647522 |
| chr1 | 198424803 | 198424853 |
| chr1 | 220253922 | 220253972 |
| chr1 | 220528469 | 220528519 |
| chr1 | 226330435 | 226330485 |
| chr1 | 231628722 | 231628772 |
| chr1 | 238339949 | 238339999 |
| chr1 | 241846302 | 241846352 |
| chr1 | 241850323 | 241850373 |
| chr1 | 243193452 | 243193502 |
| chr1 | 244214412 | 244214462 |
| chr1 | 244485834 | 244485884 |
| chr20 | 753042 | 753092 |
| chr20 | 5595008 | 5595058 |
| chr20 | 5599935 | 5599985 |
| chr20 | 13995802 | 13995852 |
| chr20 | 16599338 | 16599388 |
| chr10 | 133236728 | 133236778 |
| chr10 | 133469033 | 133469083 |
| chr10 | 133468972 | 133469022 |
| chr20 | 36878178 | 36878228 |
| chr20 | 42971427 | 42971477 |
| chr20 | 43841592 | 43841642 |
| chr20 | 43843584 | 43843634 |
| chr20 | 44759601 | 44759651 |
| chr20 | 44759384 | 44759434 |
| chr20 | 47775676 | 47775726 |
| chr20 | 52445703 | 52445753 |
| chr20 | 53157668 | 53157718 |
| chr20 | 57196000 | 57196050 |
| chr10 | 134630449 | 134630499 |
| chr10 | 134632230 | 134632280 |
| chr21 | 19277888 | 19277938 |
| chr11 | 306405 | 306455 |
| chr21 | 24930151 | 24930201 |
| chr21 | 36391827 | 36391877 |
| chr21 | 39828507 | 39828557 |
| chr21 | 39872516 | 39872566 |
| chr11 | 738590 | 738640 |
| chr21 | 43242867 | 43242917 |
| chr21 | 43242980 | 43243030 |
| chr21 | 43244130 | 43244180 |
| chr21 | 43275985 | 43276035 |
| chr22 | 14693356 | 14693406 |
| chr22 | 23003129 | 23003179 |
| chr22 | 28302489 | 28302539 |
| chr22 | 28303424 | 28303474 |
| chr22 | 34392887 | 34392937 |
| chr22 | 34393443 | 34393493 |
| chr22 | 34395099 | 34395149 |
| chr22 | 39346774 | 39346824 |
| chr22 | 41298475 | 41298525 |
| chr22 | 46080758 | 46080808 |
| chr22 | 46535717 | 46535767 |
| chr22 | 48127187 | 48127237 |
| chr22 | 49213880 | 49213930 |
| chrX | 2315697 | 2315747 |
| chrX | 2317816 | 2317866 |
| chrX | 2483102 | 2483152 |
| chrX | 2686180 | 2686230 |
| chrX | 3918521 | 3918571 |
| chrX | 4779081 | 4779131 |
| chrX | 12811361 | 12811411 |
| chrX | 16147428 | 16147478 |
| chrX | 30162570 | 30162620 |
| chrX | 39697101 | 39697151 |
| chrX | 43388947 | 43388997 |
| chrX | 43525237 | 43525287 |
| chrX | 45164723 | 45164773 |
| chrX | 45437701 | 45437751 |
| chrX | 46283909 | 46283959 |
| chr11 | 7956114 | 7956164 |
| chr11 | 7955709 | 7955759 |
| chrX | 63250412 | 63250462 |
| chrX | 69423204 | 69423254 |
| chrX | 96391570 | 96391620 |
| chrX | 99098551 | 99098601 |
| chrX | 99100106 | 99100156 |
| chrX | 109566684 | 109566734 |
| chrX | 119232011 | 119232061 |
| chrX | 122066625 | 122066675 |
| chr11 | 14162704 | 14162754 |
| chrY | 13568592 | 13568642 |
| chrY | 13804461 | 13804511 |
| chrY | 17208727 | 17208777 |
| chr2 | 11313885 | 11313935 |
| chr2 | 16116695 | 16116745 |
| chr2 | 24585626 | 24585676 |
| chr2 | 25379603 | 25379653 |
| chr11 | 18644797 | 18644847 |
| chr2 | 33805759 | 33805809 |
| chr2 | 36847108 | 36847158 |
| chr2 | 43092909 | 43092959 |
| chr2 | 45792265 | 45792315 |
| chr2 | 47529103 | 47529153 |
| chr2 | 47899899 | 47899949 |
| chr2 | 50079611 | 50079661 |
| chr2 | 81186594 | 81186644 |
| chr2 | 96585806 | 96585856 |
| chr2 | 96591337 | 96591387 |
| chr2 | 106397094 | 106397144 |
| chr2 | 108481066 | 108481116 |
| chr2 | 108633115 | 108633165 |
| chr2 | 109334729 | 109334779 |
| chr2 | 122555188 | 122555238 |
| chr2 | 128928168 | 128928218 |
| chr2 | 134840943 | 134840993 |
| chr2 | 151936778 | 151936828 |
| chr2 | 157492741 | 157492791 |
| chr2 | 164144919 | 164144969 |
| chr2 | 172971467 | 172971517 |
| chr2 | 174374565 | 174374615 |
| chr2 | 174376460 | 174376510 |
| chr2 | 175751791 | 175751841 |
| chr2 | 212657826 | 212657876 |
| chr2 | 228820072 | 228820122 |
| chr2 | 238661398 | 238661448 |
| chr11 | 46553554 | 46553604 |
| chr3 | 5127176 | 5127226 |
| chr3 | 5276446 | 5276496 |
| chr3 | 7748688 | 7748738 |
| chr3 | 9658743 | 9658793 |
| chr3 | 9860757 | 9860807 |
| chr3 | 15413956 | 15414006 |
| chr3 | 27621952 | 27622002 |
| chr3 | 31997632 | 31997682 |
| chr3 | 36677325 | 36677375 |
| chr3 | 38347087 | 38347137 |
| chr3 | 40554525 | 40554575 |
| chr3 | 42798928 | 42798978 |
| chr3 | 47481513 | 47481563 |
| chr3 | 48242223 | 48242273 |
| chr3 | 67754767 | 67754817 |
| chr11 | 59088252 | 59088302 |
| chr3 | 101622392 | 101622442 |
| chr3 | 123349358 | 123349408 |
| chr3 | 133316318 | 133316368 |
| chr3 | 143296932 | 143296982 |
| chr3 | 156992685 | 156992735 |
| chr11 | 61891217 | 61891267 |
| chr3 | 173934752 | 173934802 |
| chr3 | 195615410 | 195615460 |
| chr3 | 197176409 | 197176459 |
| chr3 | 197200014 | 197200064 |
| chr3 | 197220210 | 197220260 |
| chr3 | 197356836 | 197356886 |
| chr3 | 197396619 | 197396669 |
| chr3 | 197497119 | 197497169 |
| chr3 | 197499422 | 197499472 |
| chr3 | 197897775 | 197897825 |
| chr4 | 8686281 | 8686331 |
| chr4 | 10304685 | 10304735 |
| chr4 | 17325347 | 17325397 |
| chr4 | 24615859 | 24615909 |
| chr4 | 25370726 | 25370776 |
| chr4 | 29142097 | 29142147 |
| chr4 | 32548735 | 32548785 |
| chr4 | 57541876 | 57541926 |
| chr4 | 63843319 | 63843369 |
| chr4 | 84107540 | 84107590 |
| chr4 | 86169587 | 86169637 |
| chr4 | 88801606 | 88801656 |
| chr4 | 90664378 | 90664428 |
| chr4 | 90664171 | 90664221 |
| chr4 | 97764135 | 97764185 |
| chr4 | 100403699 | 100403749 |
| chr4 | 101166999 | 101167049 |
| chr4 | 104083333 | 104083383 |
| chr4 | 108857512 | 108857562 |
| chr4 | 126940607 | 126940657 |
| chr4 | 131310251 | 131310301 |
| chr4 | 165740357 | 165740407 |
| chr4 | 176961241 | 176961291 |
| chr4 | 186226357 | 186226407 |
| chr11 | 68206349 | 68206399 |
| chr4 | 187708047 | 187708097 |
| chr4 | 191457244 | 191457294 |
| chr5 | 8049675 | 8049725 |
| chr5 | 17468688 | 17468738 |
| chr5 | 18599684 | 18599734 |
| chr5 | 19384198 | 19384248 |
| chr5 | 27141582 | 27141632 |
| chr5 | 36637185 | 36637235 |
| chr5 | 43118264 | 43118314 |
| chr5 | 53324958 | 53325008 |
| chr5 | 53817100 | 53817150 |
| chr5 | 56513013 | 56513063 |
| chr5 | 77131402 | 77131452 |
| chr5 | 79514033 | 79514083 |
| chr5 | 81327129 | 81327179 |
| chr5 | 84847406 | 84847456 |
| chr5 | 103475119 | 103475169 |
| chr5 | 108836390 | 108836440 |
| chr10 | 14422378 | 14422428 |
| chr5 | 127168179 | 127168229 |
| chr5 | 150709491 | 150709541 |
| chr5 | 150709816 | 150709866 |
| chr5 | 157328767 | 157328817 |
| chr5 | 172216424 | 172216474 |
| chr5 | 172449811 | 172449861 |
| chr5 | 175020076 | 175020126 |
| chr5 | 180803497 | 180803547 |
| chr6 | 804608 | 804658 |
| chr11 | 77075747 | 77075797 |
| chr6 | 3894186 | 3894236 |
| chr6 | 10844331 | 10844381 |
| chr6 | 15311067 | 15311117 |
| chr6 | 15312756 | 15312806 |
| chr6 | 15802362 | 15802412 |
| chr6 | 16325023 | 16325073 |
| chr6 | 21653634 | 21653684 |
| chr6 | 27722850 | 27722900 |
| chr6 | 27956148 | 27956198 |
| chr6 | 29045975 | 29046025 |
| chr6 | 30396548 | 30396598 |
| chr6 | 31563106 | 31563156 |
| chr6 | 56628499 | 56628549 |
| chr6 | 56941147 | 56941197 |
| chr6 | 71630463 | 71630513 |
| chr6 | 76590178 | 76590228 |
| chr6 | 107344884 | 107344934 |
| chr6 | 127675438 | 127675488 |
| chr6 | 132163918 | 132163968 |
| chr11 | 97474790 | 97474840 |
| chr6 | 142271154 | 142271204 |
| chr6 | 147750043 | 147750093 |
| chr6 | 151565426 | 151565476 |
| chr6 | 158175191 | 158175241 |
| chr6 | 160226996 | 160227046 |
| chr11 | 101881604 | 101881654 |
| chr11 | 101974556 | 101974606 |
| chr6 | 162861843 | 162861893 |
| chr7 | 1185198 | 1185248 |
| chr7 | 2993174 | 2993224 |
| chr7 | 5008567 | 5008617 |
| chr7 | 6353292 | 6353342 |
| chr7 | 6354450 | 6354500 |
| chr7 | 26120268 | 26120318 |
| chr7 | 29105569 | 29105619 |
| chr7 | 37443951 | 37444001 |
| chr7 | 39931293 | 39931343 |
| chr7 | 44768957 | 44769007 |
| chr7 | 46168987 | 46169037 |
| chr7 | 55193681 | 55193731 |
| chr7 | 67551002 | 67551052 |
| chr7 | 69979513 | 69979563 |
| chr7 | 70312790 | 70312840 |
| chr7 | 71440322 | 71440372 |
| chr7 | 72217223 | 72217273 |
| chr7 | 73277695 | 73277745 |
| chr7 | 73279639 | 73279689 |
| chr7 | 75046828 | 75046878 |
| chr7 | 75427206 | 75427256 |
| chr7 | 75428470 | 75428520 |
| chr7 | 77777815 | 77777865 |
| chr7 | 78857920 | 78857970 |
| chr7 | 100142002 | 100142052 |
| chr7 | 100476168 | 100476218 |
| chr10 | 17435115 | 17435165 |
| chr11 | 118264175 | 118264225 |
| chr7 | 129418230 | 129418280 |
| chr7 | 137307934 | 137307984 |
| chr7 | 137358824 | 137358874 |
| chr7 | 137412552 | 137412602 |
| chr7 | 139593021 | 139593071 |
| chr7 | 141101242 | 141101292 |
| chr7 | 147241919 | 147241969 |
| chr7 | 154218861 | 154218911 |
| chr7 | 156303862 | 156303912 |
| chr10 | 17966490 | 17966540 |
| chr8 | 1736253 | 1736303 |
| chr8 | 1736186 | 1736236 |
| chr8 | 1737344 | 1737394 |
| chr10 | 18213400 | 18213450 |
| chr8 | 2987047 | 2987097 |
| chr8 | 3970418 | 3970468 |
| chr8 | 6612418 | 6612468 |
| chr8 | 26548620 | 26548670 |
| chr8 | 30163171 | 30163221 |
| chr8 | 41686359 | 41686409 |
| chr8 | 48949646 | 48949696 |
| chr8 | 81352205 | 81352255 |
| chr8 | 90881101 | 90881151 |
| chr8 | 91586803 | 91586853 |
| chr8 | 91586500 | 91586550 |
| chr8 | 98740673 | 98740723 |
| chr8 | 129070264 | 129070314 |
| chr8 | 144233820 | 144233870 |
| chr9 | 15126155 | 15126205 |
| chr9 | 24107772 | 24107822 |
| chr9 | 37393585 | 37393635 |
| chr9 | 42396698 | 42396748 |
| chr9 | 62609127 | 62609177 |
| chr9 | 82462270 | 82462320 |
| chr9 | 86426032 | 86426082 |
| chr9 | 86448503 | 86448553 |
| chr9 | 86583986 | 86584036 |
| chr9 | 108412234 | 108412284 |
| chr9 | 109960080 | 109960130 |
| chr9 | 110993525 | 110993575 |
| chr9 | 111048161 | 111048211 |
| chr9 | 115266322 | 115266372 |
| chr9 | 133800282 | 133800332 |
| chr10 | 21686937 | 21686987 |
| chr9 | 135692103 | 135692153 |
| chr9 | 135692276 | 135692326 |
| chr12 | 8864704 | 8864754 |
| chr12 | 8875406 | 8875456 |
| chr12 | 12919904 | 12919954 |
| chr12 | 23408640 | 23408690 |
| chr12 | 38856834 | 38856884 |
| chr12 | 45981834 | 45981884 |
| chr12 | 46642368 | 46642418 |
| chr12 | 51791449 | 51791499 |
| chr12 | 66047339 | 66047389 |
| chr12 | 74427811 | 74427861 |
| chr12 | 89113498 | 89113548 |
| chr12 | 92927593 | 92927643 |
| chr10 | 30571889 | 30571939 |
| chr12 | 116967454 | 116967504 |
| chr12 | 118280951 | 118281001 |
| chr12 | 121614687 | 121614737 |
| chr12 | 121632499 | 121632549 |
| chr12 | 121837260 | 121837310 |
| chr12 | 122907697 | 122907747 |
| chr12 | 123757609 | 123757659 |
| chr12 | 126582794 | 126582844 |
| chr12 | 126784590 | 126784640 |
| chr12 | 131260958 | 131261008 |
| chr13 | 21255392 | 21255442 |
| chr13 | 22900809 | 22900859 |
| chr13 | 28345729 | 28345779 |
| chr13 | 41251960 | 41252010 |
| chr13 | 43100113 | 43100163 |
| chr13 | 44378379 | 44378429 |
| chr13 | 47974986 | 47975036 |
| chr13 | 58956760 | 58956810 |
| chr13 | 67305995 | 67306045 |
| chr13 | 67977245 | 67977295 |
| chr13 | 75385665 | 75385715 |
| chr13 | 82285955 | 82286005 |
| chr13 | 83958651 | 83958701 |
| chr13 | 84049778 | 84049828 |
| chr13 | 84732843 | 84732893 |
| chr13 | 96836049 | 96836099 |
| chr13 | 112528322 | 112528372 |
| chr14 | 19132231 | 19132281 |
| chr14 | 19656490 | 19656540 |
| chr14 | 21187554 | 21187604 |
| chr14 | 25211335 | 25211385 |
| chr14 | 32817041 | 32817091 |
| chr14 | 48339272 | 48339322 |
| chr14 | 48339865 | 48339915 |
| chr14 | 50578785 | 50578835 |
| chr14 | 52930468 | 52930518 |
| chr14 | 62105065 | 62105115 |
| chr14 | 62821335 | 62821385 |
| chr14 | 75548271 | 75548321 |
| chr14 | 80073311 | 80073361 |
| chr10 | 54128381 | 54128431 |
| chr14 | 90055443 | 90055493 |
| chr14 | 91035870 | 91035920 |
| chr15 | 23816087 | 23816137 |
| chr15 | 25064407 | 25064457 |
| chr15 | 29139022 | 29139072 |
| chr15 | 33069427 | 33069477 |
| chr15 | 39159919 | 39159969 |
| chr15 | 39602416 | 39602466 |
| chr15 | 40851693 | 40851743 |
| chr15 | 41731703 | 41731753 |
| chr15 | 48797600 | 48797650 |
| chr15 | 53435645 | 53435695 |
| chr15 | 61389910 | 61389960 |
| chr15 | 61391052 | 61391102 |
| chr15 | 65910930 | 65910980 |
| chr15 | 66031287 | 66031337 |
| chr15 | 73155124 | 73155174 |
| chr15 | 87263415 | 87263465 |
| chr15 | 99439815 | 99439865 |
| chr16 | 1934427 | 1934477 |
| chr16 | 1934980 | 1935030 |
| chr10 | 70409128 | 70409178 |
| chr16 | 2847001 | 2847051 |
| chr16 | 8162325 | 8162375 |
| chr16 | 12870144 | 12870194 |
| chr10 | 71493102 | 71493152 |
| chr16 | 17722017 | 17722067 |
| chr16 | 20540738 | 20540788 |
| chr16 | 25551808 | 25551858 |
| chr16 | 26996996 | 26997046 |
| chr16 | 27060510 | 27060560 |
| chr16 | 31316426 | 31316476 |
| chr16 | 46465470 | 46465520 |
| chr16 | 64527680 | 64527730 |
| chr16 | 69604050 | 69604100 |
| chr16 | 78878402 | 78878452 |
| chr16 | 80909534 | 80909584 |
| chr16 | 81703347 | 81703397 |
| chr16 | 85787634 | 85787684 |
| chr10 | 407098 | 407148 |
| chr17 | 259399 | 259449 |
| chr17 | 316603 | 316653 |
| chr17 | 5030486 | 5030536 |
| chr17 | 7246205 | 7246255 |
| chr17 | 7642629 | 7642679 |
| chr17 | 8087569 | 8087619 |
| chr17 | 9437562 | 9437612 |
| chr17 | 9520537 | 9520587 |
| chr17 | 16348340 | 16348390 |
| chr10 | 3737554 | 3737604 |
| chr17 | 30633815 | 30633865 |
| chr17 | 31909319 | 31909369 |
| chr17 | 37586734 | 37586784 |

Table 15BS. Repeat Category LTR8A

| | | |
|---|---|---|
| chr17 | 47630768 | 47630818 |
| chr10 | 84099718 | 84099768 |
| chr17 | 67940971 | 67941021 |
| chr17 | 74576553 | 74576603 |
| chr17 | 78838348 | 78838398 |
| chr17 | 81389895 | 81389945 |
| chr19 | 2417602 | 2417652 |
| chr19 | 2419597 | 2419647 |
| chr19 | 2904015 | 2904065 |
| chr19 | 12036997 | 12037047 |
| chr19 | 12038223 | 12038273 |
| chr19 | 12267023 | 12267073 |
| chr19 | 12268678 | 12268728 |
| chr19 | 12855135 | 12855185 |
| chr19 | 17387418 | 17387468 |
| chr19 | 45375634 | 45375684 |
| chr19 | 46997083 | 46997133 |
| chr1 | 24910672 | 24910722 |
| chr1 | 24911038 | 24911088 |
| chr1 | 110109997 | 110110047 |
| chr1 | 158155769 | 158155819 |
| chr1 | 198424803 | 198424853 |
| chr1 | 220253922 | 220253972 |
| chr1 | 220528469 | 220528519 |
| chr1 | 226330435 | 226330485 |
| chr1 | 238339949 | 238339999 |
| chr1 | 243193452 | 243193502 |
| chr1 | 244485834 | 244485884 |
| chr20 | 753042 | 753092 |
| chr20 | 5595008 | 5595058 |
| chr20 | 13995802 | 13995852 |
| chr20 | 36878178 | 36878228 |
| chr20 | 57196000 | 57196050 |
| chr21 | 19277888 | 19277938 |
| chr11 | 306405 | 306455 |
| chr11 | 738590 | 738640 |
| chr21 | 43244130 | 43244180 |
| chr22 | 39346774 | 39346824 |
| chr22 | 48127187 | 48127237 |
| chrX | 3918521 | 3918571 |
| chrX | 12811361 | 12811411 |
| chrX | 45437701 | 45437751 |
| chr11 | 7956114 | 7956164 |
| chr11 | 7955709 | 7955759 |
| chr2 | 24585626 | 24585676 |
| chr2 | 81186594 | 81186644 |
| chr2 | 106397094 | 106397144 |
| chr2 | 128928168 | 128928218 |
| chr2 | 151936778 | 151936828 |
| chr2 | 164144919 | 164144969 |
| chr2 | 238661398 | 238661448 |
| chr11 | 46553554 | 46553604 |
| chr3 | 5276446 | 5276496 |
| chr3 | 7748688 | 7748738 |
| chr3 | 9860757 | 9860807 |
| chr3 | 15413956 | 15414006 |
| chr3 | 27621952 | 27622002 |
| chr3 | 31997632 | 31997682 |
| chr3 | 40554525 | 40554575 |
| chr3 | 47481513 | 47481563 |
| chr3 | 48242223 | 48242273 |
| chr11 | 61891217 | 61891267 |
| chr3 | 197200014 | 197200064 |
| chr3 | 197220210 | 197220260 |
| chr3 | 197499422 | 197499472 |
| chr4 | 8686281 | 8686331 |
| chr4 | 10304685 | 10304735 |
| chr4 | 29142097 | 29142147 |
| chr4 | 57541876 | 57541926 |
| chr4 | 84107540 | 84107590 |
| chr4 | 90664378 | 90664428 |
| chr4 | 90664171 | 90664221 |
| chr4 | 101166999 | 101167049 |
| chr4 | 165740357 | 165740407 |
| chr4 | 176961241 | 176961291 |
| chr11 | 68206349 | 68206399 |
| chr4 | 191457244 | 191457294 |
| chr5 | 27141582 | 27141632 |
| chr5 | 43118264 | 43118314 |
| chr5 | 53324958 | 53325008 |
| chr5 | 53817100 | 53817150 |
| chr5 | 56513013 | 56513063 |
| chr5 | 157328767 | 157328817 |
| chr5 | 172216424 | 172216474 |
| chr5 | 175020076 | 175020126 |
| chr6 | 804608 | 804658 |
| chr6 | 3894186 | 3894236 |
| chr6 | 16325023 | 16325073 |
| chr6 | 21653634 | 21653684 |
| chr6 | 29045975 | 29046025 |
| chr6 | 31563106 | 31563156 |
| chr6 | 56628499 | 56628549 |
| chr6 | 71630463 | 71630513 |
| chr6 | 107344884 | 107344934 |
| chr6 | 127675438 | 127675488 |
| chr6 | 142271154 | 142271204 |
| chr6 | 147750043 | 147750093 |
| chr6 | 151565426 | 151565476 |
| chr11 | 101974556 | 101974606 |
| chr7 | 1185198 | 1185248 |
| chr7 | 2993174 | 2993224 |
| chr7 | 6354450 | 6354500 |
| chr7 | 29105569 | 29105619 |
| chr7 | 55193681 | 55193731 |
| chr7 | 69979513 | 69979563 |
| chr7 | 71440322 | 71440372 |
| chr7 | 73277695 | 73277745 |
| chr7 | 73279639 | 73279689 |
| chr7 | 75046828 | 75046878 |
| chr7 | 75427206 | 75427256 |
| chr7 | 75428470 | 75428520 |
| chr7 | 100476168 | 100476218 |
| chr7 | 141101242 | 141101292 |
| chr7 | 147241919 | 147241969 |
| chr7 | 154218861 | 154218911 |
| chr7 | 156303862 | 156303912 |
| chr8 | 1736253 | 1736303 |
| chr8 | 1736186 | 1736236 |
| chr8 | 1737344 | 1737394 |
| chr8 | 26548620 | 26548670 |
| chr8 | 41686359 | 41686409 |
| chr8 | 81352205 | 81352255 |
| chr8 | 90881101 | 90881151 |
| chr8 | 91586803 | 91586853 |
| chr8 | 91586500 | 91586550 |
| chr8 | 98740673 | 98740723 |
| chr8 | 129070264 | 129070314 |
| chr9 | 135692103 | 135692153 |
| chr9 | 135692276 | 135692326 |
| chr12 | 23408640 | 23408690 |
| chr12 | 38856834 | 38856884 |
| chr12 | 89113498 | 89113548 |
| chr12 | 126784590 | 126784640 |
| chr13 | 22900809 | 22900859 |
| chr13 | 41251960 | 41252010 |
| chr13 | 47974986 | 47975036 |
| chr13 | 82285955 | 82286005 |
| chr14 | 25211335 | 25211385 |
| chr14 | 50578785 | 50578835 |
| chr14 | 75548271 | 75548321 |
| chr14 | 80073311 | 80073361 |
| chr15 | 40851693 | 40851743 |
| chr15 | 66031287 | 66031337 |
| chr15 | 87263415 | 87263465 |
| chr16 | 17722017 | 17722067 |
| chr16 | 20540738 | 20540788 |
| chr16 | 25551808 | 25551858 |
| chr16 | 27060510 | 27060560 |
| chr16 | 69604050 | 69604100 |
| chr16 | 78878402 | 78878452 |
| chr16 | 81703347 | 81703397 |
| chr17 | 9520537 | 9520587 |
| chr17 | 30633815 | 30633865 |
| chr17 | 31909319 | 31909369 |

Table 15BT. Repeat Category LTR9

| | | |
|---|---|---|
| chr17 | 52185310 | 52185360 |
| chr17 | 65868183 | 65868233 |
| chr17 | 69674264 | 69674314 |
| chr10 | 90983283 | 90983333 |
| chr10 | 91779633 | 91779683 |
| chr10 | 92237746 | 92237796 |
| chr18 | 3320719 | 3320769 |
| chr18 | 3320816 | 3320866 |
| chr10 | 93837233 | 93837283 |
| chr18 | 6918643 | 6918693 |
| chr18 | 8926006 | 8926056 |
| chr18 | 13660988 | 13661038 |
| chr18 | 16910157 | 16910207 |
| chr18 | 27576432 | 27576482 |
| chr18 | 44030534 | 44030584 |
| chr18 | 45232942 | 45232992 |
| chr18 | 53647462 | 53647512 |
| chr18 | 67104173 | 67104223 |
| chr18 | 71905396 | 71905446 |
| chr19 | 6553460 | 6553510 |
| chr19 | 6555861 | 6555911 |
| chr19 | 6836367 | 6836417 |
| chr19 | 6835869 | 6835919 |
| chr19 | 6881254 | 6881304 |
| chr19 | 9204350 | 9204400 |
| chr19 | 13510005 | 13510055 |
| chr19 | 14627649 | 14627699 |
| chr19 | 14647844 | 14647894 |
| chr19 | 14648466 | 14648516 |
| chr19 | 14648655 | 14648705 |
| chr19 | 14730921 | 14730971 |
| chr19 | 14775974 | 14776024 |
| chr19 | 16109033 | 16109083 |
| chr10 | 103610338 | 103610388 |
| chr19 | 18936286 | 18936336 |
| chr19 | 18937390 | 18937440 |
| chr19 | 21674557 | 21674607 |
| chr19 | 24128575 | 24128625 |
| chr19 | 37445850 | 37445900 |
| chr19 | 39085948 | 39085998 |
| chr19 | 49307995 | 49308045 |
| chr19 | 49308182 | 49308232 |
| chr19 | 49308398 | 49308448 |
| chr19 | 54923054 | 54923104 |
| chr19 | 55787269 | 55787319 |
| chr19 | 56442846 | 56442896 |
| chr19 | 61082548 | 61082598 |
| chr19 | 61601326 | 61601376 |
| chr1 | 7846387 | 7846437 |
| chr10 | 116714434 | 116714484 |
| chr1 | 9281222 | 9281272 |
| chr1 | 12376074 | 12376124 |
| chr1 | 14093112 | 14093162 |
| chr1 | 15198503 | 15198553 |
| chr1 | 27132689 | 27132739 |
| chr1 | 39857947 | 39857997 |
| chr1 | 48043929 | 48043979 |
| chr1 | 51846084 | 51846134 |
| chr10 | 123410780 | 123410830 |
| chr1 | 55292584 | 55292634 |
| chr1 | 75611654 | 75611704 |
| chr1 | 112693661 | 112693711 |
| chr1 | 113866366 | 113866416 |
| chr1 | 158008800 | 158008850 |
| chr1 | 158162413 | 158162463 |
| chr1 | 185772310 | 185772360 |
| chr1 | 193394775 | 193394825 |
| chr1 | 204998449 | 204998499 |
| chr1 | 205083107 | 205083157 |
| chr1 | 205082948 | 205082998 |
| chr1 | 207466474 | 207466524 |
| chr1 | 216500736 | 216500786 |
| chr1 | 216500430 | 216500480 |
| chr1 | 218944298 | 218944348 |
| chr1 | 218944174 | 218944224 |
| chr1 | 220264991 | 220265041 |
| chr1 | 225819978 | 225820028 |
| chr1 | 226128214 | 226128264 |
| chr1 | 227794895 | 227794945 |
| chr1 | 229309751 | 229309801 |
| chr1 | 232632953 | 232633003 |
| chr1 | 233492800 | 233492850 |
| chr1 | 233721937 | 233721987 |
| chr10 | 131792040 | 131792090 |
| chr1 | 242390069 | 242390119 |
| chr1 | 242390126 | 242390176 |
| chr1 | 243201130 | 243201180 |
| chr20 | 4181311 | 4181361 |
| chr20 | 11387711 | 11387761 |
| chr20 | 11448629 | 11448679 |
| chr20 | 12676491 | 12676541 |
| chr20 | 12775675 | 12775725 |
| chr20 | 34665989 | 34666039 |
| chr20 | 40987783 | 40987833 |
| chr20 | 42609553 | 42609603 |
| chr20 | 42610221 | 42610271 |
| chr20 | 46837506 | 46837556 |
| chr20 | 47118528 | 47118578 |
| chr20 | 51639041 | 51639091 |
| chr20 | 52971069 | 52971119 |
| chr20 | 52971713 | 52971763 |
| chr21 | 17587571 | 17587621 |
| chr21 | 25136360 | 25136410 |
| chr21 | 31486787 | 31486837 |
| chr21 | 31869165 | 31869215 |
| chr21 | 31934483 | 31934533 |
| chr21 | 33654906 | 33654956 |
| chr21 | 34993531 | 34993581 |
| chr21 | 38413983 | 38414033 |
| chr21 | 38413841 | 38413891 |
| chr21 | 39198026 | 39198076 |
| chr21 | 44476192 | 44476242 |
| chr22 | 42670580 | 42670630 |
| chrX | 2559458 | 2559508 |
| chrX | 3105577 | 3105627 |
| chrX | 6201892 | 6201942 |
| chrX | 12581371 | 12581421 |
| chrX | 13739636 | 13739686 |
| chrX | 15127494 | 15127544 |
| chrX | 26033665 | 26033715 |
| chrX | 30393950 | 30394000 |
| chr11 | 6308826 | 6308876 |
| chrX | 45164723 | 45164773 |
| chrX | 87783484 | 87783534 |
| chrX | 87789407 | 87789457 |
| chrX | 92465470 | 92465520 |
| chrX | 101884039 | 101884089 |
| chr11 | 9758708 | 9758758 |
| chrX | 107984908 | 107984958 |
| chrX | 111624328 | 111624378 |
| chrX | 121651307 | 121651357 |
| chrX | 138718853 | 138718903 |
| chrY | 2532649 | 2532699 |
| chrY | 2981340 | 2981390 |
| chrY | 4174253 | 4174303 |
| chrY | 20100026 | 20100076 |
| chr2 | 24975120 | 24975170 |
| chr2 | 30388405 | 30388455 |
| chr2 | 38309454 | 38309504 |
| chr2 | 53848462 | 53848512 |
| chr2 | 64282881 | 64282931 |
| chr2 | 65933219 | 65933269 |
| chr2 | 70822185 | 70822235 |
| chr2 | 74393201 | 74393251 |
| chr2 | 78359108 | 78359158 |
| chr2 | 81600536 | 81600586 |
| chr2 | 89153971 | 89154021 |
| chr2 | 102989106 | 102989156 |
| chr2 | 111541407 | 111541457 |
| chr2 | 119414340 | 119414390 |
| chr2 | 128676950 | 128677000 |
| chr2 | 143675794 | 143675844 |
| chr2 | 143717275 | 143717325 |
| chr2 | 155362324 | 155362374 |
| chr2 | 166052823 | 166052873 |
| chr2 | 191186576 | 191186626 |
| chr2 | 192545092 | 192545142 |
| chr2 | 192571583 | 192571633 |
| chr2 | 203746179 | 203746229 |
| chr2 | 207007232 | 207007282 |
| chr11 | 43940243 | 43940293 |
| chr2 | 224791065 | 224791115 |
| chr2 | 231229906 | 231229956 |
| chr3 | 2997588 | 2997638 |
| chr3 | 3259320 | 3259370 |
| chr10 | 12408246 | 12408296 |
| chr3 | 25960832 | 25960882 |
| chr3 | 43419361 | 43419411 |
| chr3 | 45686387 | 45686437 |
| chr3 | 48242223 | 48242273 |
| chr3 | 52160154 | 52160204 |
| chr3 | 77443094 | 77443144 |
| chr11 | 58438926 | 58438976 |
| chr3 | 80372275 | 80372325 |
| chr11 | 58502192 | 58502242 |
| chr11 | 58597031 | 58597081 |
| chr11 | 58640822 | 58640872 |
| chr3 | 97697523 | 97697573 |
| chr3 | 99364901 | 99364951 |
| chr3 | 121304032 | 121304082 |
| chr3 | 130487602 | 130487652 |
| chr3 | 136630758 | 136630808 |
| chr3 | 141755168 | 141755218 |
| chr3 | 155101775 | 155101825 |
| chr3 | 173715449 | 173715499 |
| chr3 | 181920034 | 181920084 |
| chr4 | 8245500 | 8245550 |
| chr4 | 13456119 | 13456169 |
| chr4 | 40159666 | 40159716 |
| chr4 | 40169083 | 40169133 |
| chr4 | 56118613 | 56118663 |
| chr4 | 74230105 | 74230155 |
| chr4 | 84513306 | 84513356 |
| chr4 | 84574672 | 84574722 |
| chr4 | 84575486 | 84575536 |
| chr4 | 110767182 | 110767232 |
| chr4 | 111014484 | 111014534 |
| chr4 | 129111573 | 129111623 |
| chr4 | 130548050 | 130548100 |
| chr4 | 134756686 | 134756736 |
| chr4 | 146033725 | 146033775 |
| chr4 | 157736172 | 157736222 |
| chr4 | 162750729 | 162750779 |
| chr4 | 169315695 | 169315745 |
| chr4 | 174159601 | 174159651 |
| chr4 | 177519833 | 177519883 |
| chr4 | 178242771 | 178242821 |
| chr4 | 185868320 | 185868370 |
| chr4 | 185868686 | 185868736 |
| chr4 | 185868922 | 185868972 |
| chr4 | 186552758 | 186552808 |
| chr4 | 186552855 | 186552905 |
| chr4 | 190618920 | 190618970 |
| chr5 | 12714391 | 12714441 |
| chr5 | 12715678 | 12715728 |
| chr5 | 13888563 | 13888613 |
| chr5 | 14950750 | 14950800 |
| chr5 | 23271333 | 23271383 |
| chr5 | 31633753 | 31633803 |
| chr5 | 32958586 | 32958636 |
| chr5 | 38706031 | 38706081 |
| chr5 | 54518737 | 54518787 |
| chr5 | 72517017 | 72517067 |
| chr5 | 76268908 | 76268958 |
| chr5 | 93774659 | 93774709 |
| chr5 | 109800039 | 109800089 |
| chr11 | 71858634 | 71858684 |
| chr5 | 126153252 | 126153302 |
| chr5 | 131654018 | 131654068 |
| chr5 | 133753936 | 133753986 |
| chr5 | 140863161 | 140863211 |
| chr5 | 151117545 | 151117595 |
| chr5 | 151526252 | 151526302 |
| chr11 | 74320868 | 74320918 |
| chr5 | 157127398 | 157127448 |
| chr5 | 157127970 | 157128020 |
| chr5 | 157374093 | 157374143 |
| chr5 | 159150381 | 159150431 |
| chr5 | 173372337 | 173372387 |
| chr11 | 77189673 | 77189723 |
| chr11 | 77189811 | 77189861 |
| chr6 | 10784560 | 10784610 |
| chr6 | 15142299 | 15142349 |
| chr6 | 18615023 | 18615073 |
| chr6 | 21762273 | 21762323 |
| chr6 | 30949092 | 30949142 |
| chr6 | 30949432 | 30949482 |
| chr6 | 30949826 | 30949876 |
| chr6 | 37439162 | 37439212 |
| chr6 | 54305013 | 54305063 |
| chr6 | 64100292 | 64100342 |
| chr6 | 70327681 | 70327731 |
| chr6 | 118946792 | 118946842 |
| chr6 | 119868884 | 119868934 |
| chr6 | 123152233 | 123152283 |
| chr11 | 97382143 | 97382193 |
| chr6 | 138257269 | 138257319 |
| chr6 | 141450396 | 141450446 |
| chr6 | 147750043 | 147750093 |
| chr6 | 151869920 | 151869970 |
| chr6 | 156712221 | 156712271 |
| chr6 | 159238556 | 159238606 |
| chr10 | 15936087 | 15936137 |
| chr7 | 3824292 | 3824342 |
| chr7 | 4315517 | 4315567 |
| chr7 | 6994552 | 6994602 |
| chr7 | 7500601 | 7500651 |
| chr7 | 7500936 | 7500986 |
| chr7 | 8973781 | 8973831 |
| chr7 | 10692589 | 10692639 |
| chr7 | 21880835 | 21880885 |
| chr7 | 32689416 | 32689466 |
| chr7 | 34906222 | 34906272 |
| chr7 | 43450030 | 43450080 |
| chr7 | 56394087 | 56394137 |
| chr7 | 63604713 | 63604763 |
| chr7 | 66269495 | 66269545 |
| chr7 | 66755146 | 66755196 |
| chr7 | 66937518 | 66937568 |
| chr7 | 67188081 | 67188131 |
| chr7 | 67192109 | 67192159 |
| chr7 | 69770542 | 69770592 |
| chr7 | 69963982 | 69964032 |
| chr7 | 70871785 | 70871835 |
| chr7 | 72989261 | 72989311 |
| chr7 | 78210486 | 78210536 |
| chr7 | 101269111 | 101269161 |
| chr7 | 105745327 | 105745377 |
| chr7 | 120631326 | 120631376 |
| chr7 | 122110183 | 122110233 |
| chr7 | 140575899 | 140575949 |
| chr7 | 141357856 | 141357906 |
| chr7 | 144174277 | 144174327 |
| chr7 | 147590764 | 147590814 |
| chr7 | 148079549 | 148079599 |
| chr7 | 152519241 | 152519291 |
| chr8 | 16396670 | 16396720 |
| chr8 | 26548620 | 26548670 |
| chr8 | 26975212 | 26975262 |
| chr8 | 29949657 | 29949707 |
| chr8 | 34331248 | 34331298 |
| chr8 | 36011683 | 36011733 |
| chr8 | 39182303 | 39182353 |
| chr8 | 50581777 | 50581827 |
| chr8 | 52312750 | 52312800 |
| chr11 | 125281717 | 125281767 |
| chr8 | 58172206 | 58172256 |
| chr8 | 58172838 | 58172888 |
| chr8 | 58176618 | 58176668 |
| chr8 | 59414931 | 59414981 |
| chr8 | 69612615 | 69612665 |
| chr8 | 69810987 | 69811037 |
| chr8 | 74993381 | 74993431 |
| chr8 | 91560020 | 91560070 |
| chr8 | 105252639 | 105252689 |
| chr8 | 112572399 | 112572449 |
| chr10 | 19046603 | 19046653 |
| chr8 | 121524096 | 121524146 |
| chr8 | 124448649 | 124448699 |
| chr8 | 127896769 | 127896819 |
| chr8 | 132478742 | 132478792 |
| chr8 | 134741678 | 134741728 |
| chr9 | 11055094 | 11055144 |
| chr9 | 13228452 | 13228502 |
| chr9 | 14820611 | 14820661 |
| chr9 | 71697387 | 71697437 |
| chr9 | 74614968 | 74615018 |
| chr9 | 75065584 | 75065634 |
| chr9 | 75658626 | 75658676 |
| chr9 | 88994622 | 88994672 |
| chr9 | 96015668 | 96015718 |
| chr9 | 100782936 | 100782986 |
| chr9 | 110011174 | 110011224 |
| chr9 | 129509269 | 129509319 |
| chr10 | 21680676 | 21680726 |
| chr5 | 69006205 | 69006255 |
| chr5 | 69006678 | 69006728 |
| chr5 | 69007232 | 69007282 |
| chr5 | 69007287 | 69007337 |
| chr5 | 70521542 | 70521592 |
| chr5 | 70522244 | 70522294 |
| chr5 | 70522471 | 70522521 |
| chr10 | 22580498 | 22580548 |
| chr12 | 9677235 | 9677285 |
| chr12 | 9827420 | 9827470 |
| chr12 | 12132185 | 12132235 |
| chr12 | 14710205 | 14710255 |
| chr12 | 28874937 | 28874987 |
| chr12 | 31696108 | 31696158 |
| chr12 | 32066592 | 32066642 |
| chr12 | 33027763 | 33027813 |
| chr12 | 39687416 | 39687466 |
| chr12 | 48916670 | 48916720 |
| chr12 | 62417673 | 62417723 |
| chr12 | 67796149 | 67796199 |
| chr12 | 75768963 | 75769013 |
| chr12 | 84843656 | 84843706 |
| chr12 | 91073369 | 91073419 |
| chr12 | 103986948 | 103986998 |
| chr12 | 103989301 | 103989351 |
| chr10 | 30446725 | 30446775 |
| chr12 | 107670719 | 107670769 |
| chr12 | 118227816 | 118227866 |
| chr10 | 32525560 | 32525610 |
| chr12 | 126842488 | 126842538 |
| chr10 | 34476656 | 34476706 |
| chr12 | 129786354 | 129786404 |
| chr13 | 19297681 | 19297731 |
| chr13 | 21271904 | 21271954 |
| chr10 | 35993736 | 35993786 |
| chr13 | 23370363 | 23370413 |
| chr13 | 29760631 | 29760681 |
| chr13 | 34739212 | 34739262 |
| chr13 | 35134322 | 35134372 |
| chr13 | 44849427 | 44849477 |
| chr13 | 48371143 | 48371193 |
| chr13 | 58658405 | 58658455 |
| chr13 | 63830569 | 63830619 |
| chr13 | 64140727 | 64140777 |
| chr13 | 69394667 | 69394717 |
| chr13 | 76140648 | 76140698 |
| chr13 | 95392261 | 95392311 |
| chr13 | 108920104 | 108920154 |
| chr14 | 19766835 | 19766885 |
| chr14 | 36681991 | 36682041 |
| chr14 | 44522908 | 44522958 |
| chr14 | 48011068 | 48011118 |
| chr14 | 50929235 | 50929285 |
| chr14 | 59757895 | 59757945 |
| chr14 | 60315992 | 60316042 |
| chr14 | 62205887 | 62205937 |
| chr14 | 64383618 | 64383668 |
| chr14 | 66385315 | 66385365 |
| chr14 | 80708678 | 80708728 |
| chr14 | 94151850 | 94151900 |
| chr10 | 57926554 | 57926604 |
| chr10 | 2318838 | 2318888 |
| chr15 | 43557565 | 43557615 |
| chr15 | 50753758 | 50753808 |
| chr15 | 78394988 | 78395038 |
| chr15 | 81126123 | 81126173 |
| chr15 | 83050979 | 83051029 |
| chr15 | 89641435 | 89641485 |
| chr15 | 91743094 | 91743144 |
| chr15 | 96672873 | 96672923 |
| chr16 | 7864320 | 7864370 |
| chr16 | 8159255 | 8159305 |
| chr16 | 11696445 | 11696495 |
| chr16 | 11697535 | 11697585 |
| chr16 | 13840226 | 13840276 |
| chr16 | 24456875 | 24456925 |
| chr16 | 24473054 | 24473104 |
| chr16 | 35309526 | 35309576 |
| chr16 | 59177054 | 59177104 |
| chr16 | 63846061 | 63846111 |
| chr16 | 74681162 | 74681212 |
| chr16 | 74967722 | 74967772 |
| chr16 | 79670224 | 79670274 |
| chr16 | 83660181 | 83660231 |
| chr17 | 7244614 | 7244664 |
| chr17 | 12324096 | 12324146 |
| chr17 | 12823133 | 12823183 |
| chr17 | 12824588 | 12824638 |
| chr17 | 16649836 | 16649886 |
| chr17 | 21579439 | 21579489 |
| chr17 | 26410035 | 26410085 |
| chr17 | 30114599 | 30114649 |

Table 15BU. Repeat Category LTR9B

| | | |
|---|---|---|
| chr10 | 90983283 | 90983333 |
| chr18 | 13660988 | 13661038 |
| chr18 | 16910157 | 16910207 |
| chr18 | 27576432 | 27576482 |
| chr1 | 39857947 | 39857997 |
| chr1 | 55292584 | 55292634 |
| chr1 | 185772310 | 185772360 |
| chr1 | 193394775 | 193394825 |
| chr1 | 204998449 | 204998499 |
| chr1 | 205083107 | 205083157 |
| chr1 | 205082948 | 205082998 |
| chr1 | 225819978 | 225820028 |
| chr1 | 229309751 | 229309801 |
| chr1 | 233721937 | 233721987 |
| chr1 | 242390069 | 242390119 |
| chr1 | 242390126 | 242390176 |
| chr20 | 12676491 | 12676541 |
| chr20 | 34665989 | 34666039 |
| chr20 | 42609553 | 42609603 |
| chr20 | 42610221 | 42610271 |
| chr20 | 46837506 | 46837556 |
| chr20 | 47118528 | 47118578 |
| chr20 | 52971069 | 52971119 |
| chr20 | 52971713 | 52971763 |
| chr21 | 17587571 | 17587621 |
| chr21 | 25136360 | 25136410 |
| chr21 | 31934483 | 31934533 |
| chr21 | 39198026 | 39198076 |
| chr21 | 44476192 | 44476242 |
| chrX | 3105577 | 3105627 |
| chrX | 26033665 | 26033715 |
| chrX | 30393950 | 30394000 |
| chrX | 87783484 | 87783534 |
| chrX | 87789407 | 87789457 |
| chrX | 101884039 | 101884089 |
| chrX | 107984908 | 107984958 |
| chrX | 111624328 | 111624378 |
| chrX | 121651307 | 121651357 |
| chrX | 138718853 | 138718903 |
| chrY | 2532649 | 2532699 |
| chrY | 2981340 | 2981390 |
| chrY | 4174253 | 4174303 |
| chrY | 20100026 | 20100076 |
| chr2 | 30388405 | 30388455 |
| chr2 | 53848462 | 53848512 |
| chr2 | 89153971 | 89154021 |
| chr2 | 111541407 | 111541457 |
| chr2 | 143675794 | 143675844 |
| chr2 | 191186576 | 191186626 |
| chr2 | 192571583 | 192571633 |
| chr2 | 203746179 | 203746229 |
| chr2 | 207007232 | 207007282 |
| chr10 | 12408246 | 12408296 |
| chr3 | 48242223 | 48242273 |
| chr3 | 97697523 | 97697573 |
| chr3 | 121304032 | 121304082 |
| chr3 | 136630758 | 136630808 |
| chr3 | 155101775 | 155101825 |
| chr3 | 173715449 | 173715499 |
| chr4 | 8245500 | 8245550 |
| chr4 | 74230105 | 74230155 |
| chr4 | 84574672 | 84574722 |
| chr4 | 84575486 | 84575536 |
| chr4 | 110767182 | 110767232 |
| chr4 | 111014484 | 111014534 |
| chr4 | 134756686 | 134756736 |
| chr4 | 162750729 | 162750779 |
| chr4 | 169315695 | 169315745 |
| chr4 | 185868320 | 185868370 |
| chr4 | 185868686 | 185868736 |
| chr4 | 185868922 | 185868972 |
| chr4 | 186552758 | 186552808 |
| chr4 | 186552855 | 186552905 |
| chr5 | 12714391 | 12714441 |
| chr5 | 12715678 | 12715728 |
| chr5 | 13888563 | 13888613 |
| chr5 | 23271333 | 23271383 |
| chr5 | 76268908 | 76268958 |
| chr11 | 71858634 | 71858684 |
| chr5 | 140863161 | 140863211 |
| chr5 | 157127398 | 157127448 |
| chr5 | 157127970 | 157128020 |
| chr5 | 157374093 | 157374143 |
| chr5 | 159150381 | 159150431 |
| chr6 | 21762273 | 21762323 |
| chr6 | 118946792 | 118946842 |
| chr6 | 123152233 | 123152283 |
| chr6 | 138257269 | 138257319 |
| chr6 | 141450396 | 141450446 |
| chr6 | 147750043 | 147750093 |
| chr10 | 15936087 | 15936137 |
| chr7 | 6994552 | 6994602 |
| chr7 | 10692589 | 10692639 |
| chr7 | 21880835 | 21880885 |
| chr7 | 34906222 | 34906272 |
| chr7 | 43450030 | 43450080 |
| chr7 | 63604713 | 63604763 |
| chr7 | 101269111 | 101269161 |
| chr7 | 120631326 | 120631376 |
| chr7 | 144174277 | 144174327 |
| chr7 | 147590764 | 147590814 |
| chr7 | 152519241 | 152519291 |
| chr8 | 29949657 | 29949707 |
| chr8 | 69810987 | 69811037 |
| chr8 | 124448649 | 124448699 |
| chr9 | 14820611 | 14820661 |
| chr9 | 96015668 | 96015718 |
| chr12 | 9827420 | 9827470 |
| chr12 | 28874937 | 28874987 |
| chr12 | 32066592 | 32066642 |
| chr12 | 48916670 | 48916720 |
| chr12 | 67796149 | 67796199 |
| chr12 | 75768963 | 75769013 |
| chr12 | 84843656 | 84843706 |
| chr12 | 103986948 | 103986998 |
| chr12 | 103989301 | 103989351 |
| chr10 | 34476656 | 34476706 |
| chr10 | 35993736 | 35993786 |
| chr13 | 23370363 | 23370413 |
| chr13 | 29760631 | 29760681 |
| chr13 | 34739212 | 34739262 |
| chr13 | 58658405 | 58658455 |
| chr13 | 64140727 | 64140777 |
| chr14 | 19766835 | 19766885 |
| chr14 | 64383618 | 64383668 |
| chr14 | 66385315 | 66385365 |
| chr14 | 80708678 | 80708728 |
| chr15 | 83050979 | 83051029 |
| chr15 | 91743094 | 91743144 |
| chr16 | 7864320 | 7864370 |
| chr16 | 74967722 | 74967772 |
| chr16 | 79670224 | 79670274 |
| chr17 | 12324096 | 12324146 |
| chr17 | 16649836 | 16649886 |
| chr17 | 26410035 | 26410085 |

Table 15BV. Repeat Category MER105

| | | |
|---|---|---|
| chr17 | 47464984 | 47465034 |
| chr17 | 76677171 | 76677221 |
| chr10 | 104986758 | 104986808 |
| chr19 | 60584278 | 60584328 |
| chr1 | 8151882 | 8151932 |
| chr10 | 123865333 | 123865383 |
| chr1 | 77854526 | 77854576 |
| chr1 | 232399307 | 232399357 |
| chr21 | 21543126 | 21543176 |
| chrX | 69046204 | 69046254 |
| chrX | 123955784 | 123955834 |
| chr2 | 131934903 | 131934953 |
| chr3 | 20792302 | 20792352 |
| chr3 | 79152643 | 79152693 |
| chr4 | 114180583 | 114180633 |
| chr5 | 5527186 | 5527236 |
| chr5 | 84028691 | 84028741 |
| chr5 | 176325998 | 176326048 |
| chr6 | 101178565 | 101178615 |
| chr7 | 1539411 | 1539461 |
| chr7 | 55030827 | 55030877 |
| chr7 | 70975239 | 70975289 |
| chr8 | 124893423 | 124893473 |
| chr9 | 34030443 | 34030493 |
| chr9 | 125157153 | 125157203 |
| chr9 | 136153727 | 136153777 |
| chr12 | 9571640 | 9571690 |
| chr12 | 9913335 | 9913385 |
| chr12 | 9913101 | 9913151 |
| chr12 | 31207018 | 31207068 |
| chr13 | 69927340 | 69927390 |
| chr13 | 76140648 | 76140698 |
| chr14 | 81009416 | 81009466 |
| chr10 | 74656398 | 74656448 |
| chr17 | 3707437 | 3707487 |
| chr17 | 3844580 | 3844630 |
| chr17 | 4678606 | 4678656 |

Table 15BW. Repeat Category MER115

| | | |
|---|---|---|
| chr17 | 40321357 | 40321407 |
| chr17 | 47237392 | 47237442 |
| chr17 | 48490858 | 48490908 |
| chr17 | 71985441 | 71985491 |
| chr17 | 73707140 | 73707190 |
| chr17 | 75901543 | 75901593 |
| chr18 | 2274999 | 2275049 |
| chr18 | 4825187 | 4825237 |
| chr18 | 5038026 | 5038076 |
| chr18 | 12293149 | 12293199 |
| chr18 | 12296070 | 12296120 |
| chr18 | 53396917 | 53396967 |
| chr18 | 53396586 | 53396636 |
| chr18 | 70064237 | 70064287 |
| chr18 | 73951577 | 73951627 |
| chr19 | 44129557 | 44129607 |
| chr10 | 107792814 | 107792864 |
| chr1 | 23569183 | 23569233 |
| chr10 | 122368143 | 122368193 |
| chr10 | 122778365 | 122778415 |
| chr1 | 45980402 | 45980452 |
| chr1 | 151547886 | 151547936 |
| chr1 | 154789294 | 154789344 |
| chr1 | 158916456 | 158916506 |
| chr1 | 178252869 | 178252919 |
| chr1 | 198608493 | 198608543 |
| chr1 | 208787046 | 208787096 |
| chr1 | 229119488 | 229119538 |
| chr1 | 230722606 | 230722656 |
| chr10 | 131462310 | 131462360 |
| chr1 | 245793997 | 245794047 |
| chr20 | 15003484 | 15003534 |
| chr20 | 15003191 | 15003241 |
| chr10 | 133108795 | 133108845 |
| chr20 | 25856108 | 25856158 |
| chr20 | 31266702 | 31266752 |
| chr20 | 31267687 | 31267737 |
| chr20 | 31267463 | 31267513 |
| chr20 | 36878178 | 36878228 |
| chr20 | 55586314 | 55586364 |
| chr20 | 58579067 | 58579117 |
| chr20 | 58819322 | 58819372 |
| chr21 | 33350187 | 33350237 |
| chr21 | 34858834 | 34858884 |
| chr21 | 46576181 | 46576231 |
| chr22 | 15615315 | 15615365 |
| chr22 | 20497056 | 20497106 |
| chr22 | 35198392 | 35198442 |
| chrX | 138437754 | 138437804 |
| chr2 | 803011 | 803061 |
| chr2 | 8490417 | 8490467 |
| chr2 | 10375995 | 10376045 |
| chr2 | 11563286 | 11563336 |
| chr2 | 15852580 | 15852630 |
| chr11 | 18742927 | 18742977 |
| chr2 | 47542655 | 47542705 |
| chr2 | 54915041 | 54915091 |
| chr10 | 11789642 | 11789692 |
| chr2 | 68948350 | 68948400 |
| chr2 | 73052612 | 73052662 |
| chr2 | 85782669 | 85782719 |
| chr2 | 114025362 | 114025412 |
| chr2 | 118378078 | 118378128 |
| chr2 | 169804603 | 169804653 |
| chr2 | 188389269 | 188389319 |
| chr2 | 193922667 | 193922717 |
| chr2 | 199825281 | 199825331 |
| chr2 | 217166079 | 217166129 |
| chr2 | 217166000 | 217166050 |
| chr2 | 240418962 | 240419012 |
| chr2 | 240540928 | 240540978 |
| chr3 | 24005272 | 24005322 |
| chr11 | 48987867 | 48987917 |
| chr3 | 56440532 | 56440582 |
| chr3 | 82545325 | 82545375 |
| chr3 | 85655475 | 85655525 |
| chr3 | 101299974 | 101300024 |
| chr3 | 137416811 | 137416861 |
| chr3 | 142496080 | 142496130 |
| chr3 | 187568478 | 187568528 |
| chr3 | 194119402 | 194119452 |
| chr3 | 198052923 | 198052973 |
| chr4 | 80812226 | 80812276 |
| chr4 | 90078346 | 90078396 |
| chr4 | 110014998 | 110015048 |
| chr4 | 180358689 | 180358739 |
| chr5 | 15775473 | 15775523 |
| chr5 | 66648730 | 66648780 |
| chr5 | 130524278 | 130524328 |
| chr5 | 173198883 | 173198933 |
| chr6 | 8620125 | 8620175 |
| chr6 | 11416871 | 11416921 |
| chr6 | 31160130 | 31160180 |
| chr11 | 85120211 | 85120261 |
| chr6 | 158318218 | 158318268 |
| chr7 | 1212987 | 1213037 |
| chr7 | 26567365 | 26567415 |
| chr11 | 118068431 | 118068481 |
| chr11 | 118264175 | 118264225 |
| chr7 | 147575622 | 147575672 |
| chr8 | 27801189 | 27801239 |
| chr11 | 124419065 | 124419115 |
| chr8 | 39908626 | 39908676 |
| chr8 | 83973237 | 83973287 |
| chr8 | 95992948 | 95992998 |
| chr8 | 105446505 | 105446555 |
| chr8 | 126391075 | 126391125 |
| chr8 | 140744817 | 140744867 |
| chr9 | 4023200 | 4023250 |
| chr11 | 132201658 | 132201708 |
| chr9 | 36869836 | 36869886 |
| chr9 | 37475850 | 37475900 |
| chr9 | 90654106 | 90654156 |
| chr12 | 3288951 | 3289001 |
| chr12 | 78325177 | 78325227 |
| chr12 | 103802354 | 103802404 |
| chr12 | 105810948 | 105810998 |
| chr12 | 111394884 | 111394934 |
| chr12 | 114001951 | 114002001 |
| chr12 | 117993932 | 117993982 |
| chr12 | 117993804 | 117993854 |
| chr13 | 46870549 | 46870599 |
| chr13 | 47972108 | 47972158 |
| chr14 | 22548547 | 22548597 |
| chr14 | 41546017 | 41546067 |
| chr10 | 49729290 | 49729340 |
| chr14 | 62717282 | 62717332 |
| chr14 | 103259210 | 103259260 |
| chr14 | 103258930 | 103258980 |
| chr14 | 103361622 | 103361672 |
| chr14 | 103361278 | 103361328 |
| chr15 | 47088059 | 47088109 |
| chr15 | 48811201 | 48811251 |
| chr15 | 51839385 | 51839435 |
| chr15 | 75589502 | 75589552 |
| chr15 | 77054920 | 77054970 |
| chr15 | 87965106 | 87965156 |
| chr10 | 73415586 | 73415636 |
| chr16 | 85150908 | 85150958 |
| chr10 | 76307542 | 76307592 |
| chr10 | 77704876 | 77704926 |

Table 15BX. Repeat Category MER11A*

| | | |
|---|---|---|
| chr18 | 395798 | 395848 |
| chr18 | 9493375 | 9493425 |
| chr18 | 70123608 | 70123658 |
| chr1 | 46736143 | 46736193 |
| chr10 | 131391175 | 131391225 |
| chr20 | 63274055 | 63274105 |
| chr22 | 18297951 | 18298001 |
| chrX | 96535519 | 96535569 |
| chr2 | 106473008 | 106473058 |
| chr2 | 242786781 | 242786831 |
| chr11 | 47861546 | 47861596 |
| chr3 | 95004507 | 95004557 |
| chr3 | 110623912 | 110623962 |
| chr3 | 113882475 | 113882525 |
| chr3 | 150361875 | 150361925 |
| chr3 | 197586983 | 197587033 |
| chr3 | 199020693 | 199020743 |
| chr4 | 15897951 | 15898001 |
| chr4 | 99792162 | 99792212 |
| chr4 | 125843647 | 125843697 |
| chr4 | 132337813 | 132337863 |
| chr4 | 136563840 | 136563890 |
| chr4 | 157396576 | 157396626 |
| chr4 | 188199329 | 188199379 |
| chr5 | 108699221 | 108699271 |
| chr5 | 125485237 | 125485287 |
| chr5 | 163734727 | 163734777 |
| chr5 | 180365018 | 180365068 |
| chr5 | 180714324 | 180714374 |
| chr6 | 13936942 | 13936992 |
| chr6 | 25273072 | 25273122 |
| chr6 | 90489024 | 90489074 |
| chr7 | 52563479 | 52563529 |
| chr7 | 127303810 | 127303860 |
| chr7 | 127485952 | 127486002 |
| chr9 | 99527397 | 99527447 |
| chr9 | 99527074 | 99527124 |
| chr12 | 55532980 | 55533030 |
| chr13 | 43446756 | 43446806 |
| chr10 | 38275869 | 38275919 |
| chr14 | 48272762 | 48272812 |
| chr14 | 89280584 | 89280634 |
| chr17 | 3442720 | 3442770 |

Table 15BY. Repeat Category MER11B

| | | |
|---|---|---|
| chr17 | 66926261 | 66926311 |
| chr18 | 67239436 | 67239486 |
| chr18 | 73025241 | 73025291 |
| chr10 | 100682273 | 100682323 |
| chr10 | 101725121 | 101725171 |
| chr19 | 12340427 | 12340477 |
| chr19 | 24049860 | 24049910 |
| chr1 | 45456542 | 45456592 |
| chr1 | 93889011 | 93889061 |
| chr1 | 108374887 | 108374937 |
| chr1 | 169306448 | 169306498 |
| chr1 | 204560439 | 204560489 |
| chr1 | 222132360 | 222132410 |
| chr1 | 240188047 | 240188097 |
| chr22 | 44469227 | 44469277 |
| chrX | 6044455 | 6044505 |
| chrX | 15802783 | 15802833 |
| chr11 | 6061449 | 6061499 |
| chrX | 123389151 | 123389201 |
| chr2 | 115689111 | 115689161 |
| chr11 | 40856827 | 40856877 |
| chr3 | 15755242 | 15755292 |
| chr3 | 20005014 | 20005064 |
| chr5 | 127859815 | 127859865 |
| chr6 | 119421111 | 119421161 |
| chr6 | 158669263 | 158669313 |
| chr6 | 160411457 | 160411507 |
| chr6 | 160411752 | 160411802 |
| chr7 | 96382598 | 96382648 |
| chr7 | 99352952 | 99353002 |
| chr7 | 99353674 | 99353724 |
| chr7 | 127824910 | 127824960 |
| chr7 | 155800315 | 155800365 |
| chr8 | 79995327 | 79995377 |
| chr8 | 108865069 | 108865119 |
| chr9 | 23379907 | 23379957 |
| chr13 | 19507397 | 19507447 |
| chr10 | 44812206 | 44812256 |
| chr15 | 66985597 | 66985647 |
| chr10 | 71772615 | 71772665 |
| chr16 | 57249633 | 57249683 |

Table 15BZ. Repeat Category MER11C

| | | |
|---|---|---|
| chr17 | 40215259 | 40215309 |
| chr17 | 63057560 | 63057610 |
| chr10 | 88278929 | 88278979 |
| chr18 | 8612792 | 8612842 |
| chr18 | 26647254 | 26647304 |
| chr18 | 31482049 | 31482099 |
| chr18 | 41589423 | 41589473 |
| chr18 | 65973109 | 65973159 |
| chr18 | 69333811 | 69333861 |
| chr19 | 23693636 | 23693686 |
| chr19 | 34380519 | 34380569 |
| chr19 | 40104065 | 40104115 |
| chr19 | 43804696 | 43804746 |
| chr19 | 43805380 | 43805430 |
| chr19 | 51362454 | 51362504 |
| chr19 | 57101744 | 57101794 |
| chr1 | 5520901 | 5520951 |
| chr1 | 45763285 | 45763335 |
| chr1 | 103941933 | 103941983 |
| chr1 | 108430247 | 108430297 |
| chr1 | 155379372 | 155379422 |
| chr10 | 128154670 | 128154720 |
| chr1 | 217643443 | 217643493 |
| chr20 | 25506559 | 25506609 |
| chr21 | 27319436 | 27319486 |
| chr21 | 36391827 | 36391877 |
| chr21 | 39368465 | 39368515 |
| chr22 | 22623362 | 22623412 |
| chr22 | 22651922 | 22651972 |
| chr22 | 25334962 | 25335012 |
| chrX | 15802783 | 15802833 |
| chrX | 18627589 | 18627639 |
| chrX | 34176612 | 34176662 |
| chrX | 45603992 | 45604042 |
| chrX | 51568206 | 51568256 |
| chrX | 106012830 | 106012880 |
| chrX | 120632211 | 120632261 |
| chrX | 126692793 | 126692843 |
| chrX | 147351121 | 147351171 |
| chrY | 14994656 | 14994706 |
| chr2 | 4811586 | 4811636 |
| chr2 | 64008260 | 64008310 |
| chr2 | 163586144 | 163586194 |
| chr2 | 203878570 | 203878620 |
| chr2 | 211124870 | 211124920 |
| chr2 | 211126480 | 211126530 |
| chr3 | 47508017 | 47508067 |
| chr3 | 131493202 | 131493252 |
| chr3 | 184939170 | 184939220 |
| chr11 | 62854917 | 62854967 |
| chr11 | 62855792 | 62855842 |
| chr3 | 197736445 | 197736495 |
| chr4 | 6183356 | 6183406 |
| chr4 | 11732812 | 11732862 |
| chr4 | 77533636 | 77533686 |
| chr4 | 77534774 | 77534824 |
| chr4 | 98853128 | 98853178 |
| chr4 | 105228450 | 105228500 |
| chr4 | 110833796 | 110833846 |
| chr4 | 154200689 | 154200739 |
| chr11 | 67124496 | 67124546 |
| chr11 | 67386064 | 67386114 |
| chr4 | 169112576 | 169112626 |
| chr4 | 182747168 | 182747218 |
| chr5 | 19720869 | 19720919 |
| chr5 | 33890040 | 33890090 |
| chr5 | 43071095 | 43071145 |
| chr5 | 44284681 | 44284731 |
| chr5 | 54187198 | 54187248 |
| chr5 | 73374153 | 73374203 |
| chr11 | 71188457 | 71188507 |
| chr5 | 107686727 | 107686777 |
| chr5 | 142827330 | 142827380 |
| chr6 | 2873253 | 2873303 |
| chr6 | 11036445 | 11036495 |
| chr6 | 30838385 | 30838435 |
| chr6 | 33931078 | 33931128 |
| chr6 | 57431704 | 57431754 |
| chr6 | 106108380 | 106108430 |
| chr11 | 94021425 | 94021475 |
| chr6 | 114665355 | 114665405 |
| chr6 | 114665210 | 114665260 |
| chr6 | 115056135 | 115056185 |
| chr6 | 115807598 | 115807648 |
| chr6 | 119856578 | 119856628 |
| chr6 | 145998698 | 145998748 |
| chr7 | 7411305 | 7411355 |
| chr7 | 12545347 | 12545397 |
| chr7 | 35757640 | 35757690 |
| chr7 | 50655591 | 50655641 |
| chr7 | 56073557 | 56073607 |
| chr7 | 63253664 | 63253714 |
| chr7 | 79817993 | 79818043 |
| chr7 | 97114553 | 97114603 |
| chr7 | 104898292 | 104898342 |
| chr7 | 108468537 | 108468587 |
| chr7 | 111450719 | 111450769 |
| chr8 | 13566180 | 13566230 |
| chr8 | 56035916 | 56035966 |
| chr8 | 63035667 | 63035717 |
| chr8 | 80032401 | 80032451 |
| chr8 | 82709657 | 82709707 |
| chr8 | 144227485 | 144227535 |
| chr9 | 23379197 | 23379247 |
| chr9 | 23379907 | 23379957 |
| chr9 | 24229494 | 24229544 |
| chr9 | 63360136 | 63360186 |
| chr9 | 78444516 | 78444566 |
| chr9 | 82114052 | 82114102 |
| chr9 | 89732147 | 89732197 |
| chr12 | 30218365 | 30218415 |
| chr12 | 82998661 | 82998711 |
| chr12 | 83004551 | 83004601 |
| chr12 | 120131073 | 120131123 |
| chr13 | 24043113 | 24043163 |
| chr13 | 46580598 | 46580648 |
| chr10 | 38720528 | 38720578 |
| chr13 | 86994951 | 86995001 |
| chr14 | 20189565 | 20189615 |
| chr14 | 39999597 | 39999647 |
| chr14 | 53630908 | 53630958 |
| chr14 | 61238264 | 61238314 |
| chr10 | 56103894 | 56103944 |
| chr14 | 105157464 | 105157514 |
| chr15 | 22126981 | 22127031 |
| chr15 | 22297882 | 22297932 |
| chr15 | 22335025 | 22335075 |
| chr15 | 22966262 | 22966312 |
| chr15 | 85052003 | 85052053 |
| chr15 | 90249504 | 90249554 |
| chr15 | 98825703 | 98825753 |
| chr15 | 98827228 | 98827278 |
| chr15 | 98826963 | 98827013 |
| chr16 | 3969546 | 3969596 |
| chr10 | 71146877 | 71146927 |
| chr16 | 14025531 | 14025581 |
| chr16 | 21306847 | 21306897 |
| chr10 | 78841864 | 78841914 |

Table 15CA. Repeat Category MER11D

| | | |
|---|---|---|
| chr10 | 96363355 | 96363405 |
| chr10 | 96363504 | 96363554 |
| chr19 | 48096167 | 48096217 |
| chrX | 7533178 | 7533228 |
| chrX | 41918905 | 41918955 |
| chrX | 147339823 | 147339873 |
| chr2 | 101481794 | 101481844 |
| chr2 | 211498285 | 211498335 |
| chr3 | 20937962 | 20938012 |
| chr4 | 59600431 | 59600481 |
| chr4 | 88671024 | 88671074 |
| chr4 | 137244525 | 137244575 |
| chr4 | 161755105 | 161755155 |
| chr5 | 153480858 | 153480908 |
| chr5 | 176353627 | 176353677 |
| chr6 | 49515803 | 49515853 |
| chr6 | 49523035 | 49523085 |
| chr6 | 66395799 | 66395849 |
| chr6 | 121344741 | 121344791 |
| chr6 | 121351123 | 121351173 |
| chr6 | 145435536 | 145435586 |
| chr6 | 158230742 | 158230792 |
| chr7 | 2212051 | 2212101 |
| chr7 | 30290910 | 30290960 |
| chr7 | 86955980 | 86956030 |
| chr7 | 86956387 | 86956437 |
| chr7 | 93112121 | 93112171 |
| chr7 | 96838755 | 96838805 |
| chr8 | 125935178 | 125935228 |
| chr8 | 135257317 | 135257367 |
| chr9 | 3100923 | 3100973 |
| chr12 | 110844055 | 110844105 |
| chr13 | 21356076 | 21356126 |
| chr14 | 45989898 | 45989948 |
| chr10 | 54621911 | 54621961 |
| chr14 | 94878274 | 94878324 |
| chr17 | 7272463 | 7272513 |

Table 15CB. Repeat Category MER34B

| | | |
|---|---|---|
| chr17 | 50772018 | 50772068 |
| chr10 | 85528575 | 85528625 |
| chr17 | 57252259 | 57252309 |
| chr17 | 62889836 | 62889886 |
| chr17 | 75209408 | 75209458 |
| chr17 | 79478135 | 79478185 |
| chr18 | 55210168 | 55210218 |
| chr19 | 7461027 | 7461077 |
| chr19 | 7461625 | 7461675 |
| chr10 | 118414968 | 118415018 |
| chr1 | 17188473 | 17188523 |
| chr1 | 23828344 | 23828394 |
| chr1 | 30900522 | 30900572 |
| chr1 | 45310060 | 45310110 |
| chr1 | 61877136 | 61877186 |
| chr1 | 112733012 | 112733062 |
| chr1 | 112862899 | 112862949 |
| chr1 | 201129540 | 201129590 |
| chr1 | 225634096 | 225634146 |
| chr20 | 23045760 | 23045810 |
| chr10 | 133729744 | 133729794 |
| chr11 | 328409 | 328459 |
| chr21 | 44550269 | 44550319 |
| chr22 | 19716208 | 19716258 |
| chr22 | 31819188 | 31819238 |
| chrX | 2633634 | 2633684 |
| chrX | 15301217 | 15301267 |
| chrX | 15348726 | 15348776 |
| chrX | 35358799 | 35358849 |
| chrX | 42427669 | 42427719 |
| chrX | 88072742 | 88072792 |
| chrX | 88082370 | 88082420 |
| chrX | 111818994 | 111819044 |
| chrX | 144620279 | 144620329 |
| chr2 | 1366082 | 1366132 |
| chr2 | 1365917 | 1365967 |
| chr2 | 27203643 | 27203693 |
| chr2 | 42702253 | 42702303 |
| chr2 | 47266649 | 47266699 |
| chr2 | 47653771 | 47653821 |
| chr2 | 62553370 | 62553420 |
| chr2 | 70456953 | 70457003 |
| chr2 | 70458976 | 70459026 |
| chr2 | 82305011 | 82305061 |
| chr2 | 233657661 | 233657711 |
| chr2 | 239143385 | 239143435 |
| chr2 | 243024320 | 243024370 |
| chr3 | 20086557 | 20086607 |
| chr3 | 40266232 | 40266282 |
| chr3 | 61224280 | 61224330 |
| chr3 | 63790429 | 63790479 |
| chr3 | 83263423 | 83263473 |
| chr3 | 99178056 | 99178106 |
| chr3 | 105560607 | 105560657 |
| chr3 | 149844097 | 149844147 |
| chr3 | 161143719 | 161143769 |
| chr3 | 168907006 | 168907056 |
| chr3 | 173064249 | 173064299 |
| chr3 | 176366136 | 176366186 |
| chr4 | 764196 | 764246 |
| chr4 | 2807281 | 2807331 |
| chr4 | 2808633 | 2808683 |
| chr4 | 4271387 | 4271437 |
| chr4 | 16756591 | 16756641 |
| chr11 | 65027004 | 65027054 |
| chr4 | 57394558 | 57394608 |
| chr4 | 57417782 | 57417832 |
| chr4 | 61590133 | 61590183 |
| chr4 | 68216689 | 68216739 |
| chr4 | 144736093 | 144736143 |
| chr4 | 162370813 | 162370863 |
| chr4 | 189385448 | 189385498 |
| chr4 | 189386833 | 189386883 |
| chr5 | 105536991 | 105537041 |
| chr5 | 116194075 | 116194125 |
| chr5 | 119011110 | 119011160 |
| chr5 | 142055119 | 142055169 |
| chr5 | 149296808 | 149296858 |
| chr5 | 178555574 | 178555624 |
| chr6 | 27895395 | 27895445 |
| chr6 | 80114928 | 80114978 |
| chr6 | 110950036 | 110950086 |
| chr6 | 144629720 | 144629770 |
| chr6 | 160443747 | 160443797 |
| chr6 | 160444042 | 160444092 |
| chr6 | 166747426 | 166747476 |
| chr7 | 77220567 | 77220617 |
| chr7 | 140077267 | 140077317 |
| chr7 | 142289936 | 142289986 |
| chr7 | 147695838 | 147695888 |
| chr8 | 23059240 | 23059290 |
| chr8 | 30008238 | 30008288 |
| chr8 | 53913511 | 53913561 |
| chr10 | 19806204 | 19806254 |
| chr10 | 19808787 | 19808837 |
| chr9 | 68875769 | 68875819 |
| chr9 | 86385437 | 86385487 |
| chr9 | 110835742 | 110835792 |
| chr12 | 31389923 | 31389973 |
| chr12 | 31918372 | 31918422 |
| chr12 | 74452319 | 74452369 |
| chr12 | 116041042 | 116041092 |
| chr13 | 28345729 | 28345779 |
| chr13 | 47798908 | 47798958 |
| chr10 | 44658317 | 44658367 |
| chr10 | 44658680 | 44658730 |
| chr10 | 44658834 | 44658884 |
| chr10 | 44659150 | 44659200 |
| chr14 | 19298418 | 19298468 |
| chr14 | 63827248 | 63827298 |
| chr14 | 65575053 | 65575103 |
| chr14 | 67315861 | 67315911 |
| chr14 | 88598977 | 88599027 |
| chr16 | 9210344 | 9210394 |
| chr16 | 48295737 | 48295787 |
| chr16 | 84826354 | 84826404 |

Table 15CC. Repeat Category MER34B-int

| | | |
|---|---|---|
| chr10 | 85528575 | 85528625 |
| chr10 | 118414968 | 118415018 |
| chr1 | 30900522 | 30900572 |
| chr1 | 45310060 | 45310110 |
| chr1 | 112733012 | 112733062 |
| chr11 | 328409 | 328459 |
| chr22 | 19716208 | 19716258 |
| chrX | 15348726 | 15348776 |
| chrX | 88082370 | 88082420 |
| chr3 | 40266232 | 40266282 |
| chr3 | 63790429 | 63790479 |
| chr3 | 83263423 | 83263473 |
| chr3 | 99178056 | 99178106 |
| chr3 | 105560607 | 105560657 |
| chr3 | 149844097 | 149844147 |
| chr3 | 161143719 | 161143769 |
| chr3 | 173064249 | 173064299 |
| chr4 | 2807281 | 2807331 |
| chr4 | 2808633 | 2808683 |
| chr4 | 61590133 | 61590183 |
| chr4 | 144736093 | 144736143 |
| chr4 | 189385448 | 189385498 |
| chr4 | 189386833 | 189386883 |
| chr5 | 105536991 | 105537041 |
| chr5 | 149296808 | 149296858 |
| chr6 | 110950036 | 110950086 |
| chr7 | 140077267 | 140077317 |
| chr7 | 142289936 | 142289986 |
| chr8 | 23059240 | 23059290 |
| chr10 | 19806204 | 19806254 |
| chr10 | 19808787 | 19808837 |

Table 15CD. Repeat Category MER34C

| | | |
|---|---|---|
| chr18 | 27651163 | 27651213 |
| chr18 | 32807414 | 32807464 |
| chr19 | 505773 | 505823 |
| chr19 | 48590743 | 48590793 |
| chr1 | 2185443 | 2185493 |
| chr1 | 2185916 | 2185966 |
| chr1 | 3358006 | 3358056 |
| chr1 | 9421164 | 9421214 |
| chr10 | 121298838 | 121298888 |
| chr1 | 178048424 | 178048474 |
| chr1 | 225800182 | 225800232 |
| chr1 | 235676212 | 235676262 |
| chr1 | 244905815 | 244905865 |
| chr20 | 20793502 | 20793552 |
| chr20 | 25219964 | 25220014 |
| chr20 | 31048153 | 31048203 |
| chr21 | 33672436 | 33672486 |
| chr21 | 40038539 | 40038589 |
| chr21 | 40039052 | 40039102 |
| chr21 | 41801469 | 41801519 |
| chr21 | 42920610 | 42920660 |
| chr21 | 43273409 | 43273459 |
| chr11 | 1887412 | 1887462 |
| chr22 | 46199928 | 46199978 |
| chrX | 2409324 | 2409374 |
| chrX | 14329409 | 14329459 |
| chrX | 42073388 | 42073438 |
| chrX | 42233616 | 42233666 |
| chrX | 66549011 | 66549061 |
| chrX | 72638840 | 72638890 |
| chr2 | 2881570 | 2881620 |
| chr2 | 33603088 | 33603138 |
| chr2 | 62077281 | 62077331 |
| chr2 | 64651812 | 64651862 |
| chr2 | 64651543 | 64651593 |
| chr2 | 65334024 | 65334074 |
| chr2 | 116687468 | 116687518 |
| chr2 | 128773443 | 128773493 |
| chr2 | 231613616 | 231613666 |
| chr3 | 48102261 | 48102311 |
| chr3 | 78291680 | 78291730 |
| chr3 | 96302937 | 96302987 |
| chr3 | 106648615 | 106648665 |
| chr11 | 63512975 | 63513025 |
| chr4 | 139611876 | 139611926 |
| chr4 | 188077113 | 188077163 |
| chr4 | 188103705 | 188103755 |
| chr5 | 79637631 | 79637681 |
| chr5 | 79638442 | 79638492 |
| chr6 | 3749470 | 3749520 |
| chr6 | 10767197 | 10767247 |
| chr6 | 29670908 | 29670958 |
| chr10 | 912747 | 912797 |
| chr6 | 139614599 | 139614649 |
| chr6 | 145498033 | 145498083 |
| chr6 | 158792646 | 158792696 |
| chr7 | 283585 | 283635 |
| chr7 | 32716676 | 32716726 |
| chr7 | 32722959 | 32723009 |
| chr7 | 44773074 | 44773124 |
| chr7 | 96567310 | 96567360 |
| chr7 | 154228662 | 154228712 |
| chr7 | 158544874 | 158544924 |
| chr8 | 42133638 | 42133688 |
| chr8 | 125328895 | 125328945 |
| chr9 | 6656012 | 6656062 |
| chr9 | 75346148 | 75346198 |
| chr9 | 129840359 | 129840409 |
| chr12 | 11661097 | 11661147 |
| chr10 | 23749942 | 23749992 |
| chr12 | 25996105 | 25996155 |
| chr12 | 69180898 | 69180948 |
| chr12 | 96089450 | 96089500 |
| chr13 | 57373738 | 57373788 |
| chr10 | 48936967 | 48937017 |
| chr14 | 42271864 | 42271914 |
| chr15 | 23548830 | 23548880 |
| chr10 | 64381632 | 64381682 |
| chr10 | 64381964 | 64382014 |
| chr15 | 72004387 | 72004437 |
| chr15 | 73039491 | 73039541 |
| chr15 | 90416999 | 90417049 |
| chr10 | 69817367 | 69817417 |
| chr15 | 99003367 | 99003417 |
| chr16 | 11639845 | 11639895 |
| chr10 | 72420232 | 72420282 |
| chr16 | 65582870 | 65582920 |
| chr16 | 85148745 | 85148795 |
| chr17 | 19021621 | 19021671 |

Table 15CE. Repeat Category MER41E

| | | |
|---|---|---|
| chr18 | 40429096 | 40429146 |
| chr19 | 6093344 | 6093394 |
| chr19 | 9909357 | 9909407 |
| chr19 | 54072893 | 54072943 |
| chr19 | 54919464 | 54919514 |
| chr1 | 8853924 | 8853974 |
| chr1 | 12399761 | 12399811 |
| chr1 | 26953633 | 26953683 |
| chr1 | 26959682 | 26959732 |
| chr1 | 156263561 | 156263611 |
| chr1 | 156263349 | 156263399 |
| chr1 | 173671809 | 173671859 |
| chr1 | 173671518 | 173671568 |
| chr20 | 62865293 | 62865343 |
| chr11 | 294389 | 294439 |
| chr21 | 45290705 | 45290755 |
| chr11 | 26339496 | 26339546 |
| chr2 | 219846914 | 219846964 |
| chr3 | 101387573 | 101387623 |
| chr4 | 26230853 | 26230903 |
| chr4 | 179137300 | 179137350 |
| chr5 | 32549469 | 32549519 |
| chr5 | 108034929 | 108034979 |
| chr11 | 93760921 | 93760971 |
| chr6 | 150278259 | 150278309 |
| chr6 | 150278742 | 150278792 |
| chr6 | 153773856 | 153773906 |
| chr8 | 8788828 | 8788878 |
| chr9 | 112720735 | 112720785 |
| chr9 | 119358141 | 119358191 |
| chr12 | 82092778 | 82092828 |
| chr12 | 116967454 | 116967504 |
| chr13 | 77131165 | 77131215 |
| chr16 | 28481543 | 28481593 |
| chr16 | 28481664 | 28481714 |
| chr16 | 28494480 | 28494530 |

Table 15CF. Repeat Category MER44C

| | | |
|---|---|---|
| chr17 | 57338431 | 57338481 |
| chr17 | 66986565 | 66986615 |
| chr18 | 13172025 | 13172075 |
| chr19 | 43014963 | 43015013 |
| chr19 | 44692815 | 44692865 |
| chr19 | 58773213 | 58773263 |
| chr1 | 216980108 | 216980158 |
| chr20 | 43512813 | 43512863 |
| chr20 | 46168018 | 46168068 |
| chr20 | 61549805 | 61549855 |
| chr21 | 16202420 | 16202470 |
| chr21 | 24077042 | 24077092 |
| chrX | 70444460 | 70444510 |
| chr2 | 855052 | 855102 |
| chr2 | 32623921 | 32623971 |
| chr2 | 108965316 | 108965366 |
| chr2 | 175045479 | 175045529 |
| chr2 | 219607366 | 219607416 |
| chr2 | 232024654 | 232024704 |
| chr3 | 124478624 | 124478674 |
| chr10 | 750879 | 750929 |
| chr3 | 184229947 | 184229997 |
| chr4 | 24601112 | 24601162 |
| chr4 | 33532047 | 33532097 |
| chr4 | 107678276 | 107678326 |
| chr5 | 72129201 | 72129251 |
| chr5 | 124501633 | 124501683 |
| chr5 | 179641912 | 179641962 |
| chr6 | 414730 | 414780 |
| chr6 | 76241121 | 76241171 |
| chr6 | 167817173 | 167817223 |
| chr11 | 111352203 | 111352253 |
| chr7 | 36228486 | 36228536 |
| chr7 | 37111702 | 37111752 |
| chr11 | 115852809 | 115852859 |
| chr7 | 158017852 | 158017902 |
| chr7 | 158018711 | 158018761 |
| chr8 | 6349354 | 6349404 |
| chr8 | 61693573 | 61693623 |
| chr8 | 61693064 | 61693114 |
| chr11 | 128550972 | 128551022 |
| chr8 | 143050292 | 143050342 |
| chr9 | 70858163 | 70858213 |
| chr10 | 24433461 | 24433511 |
| chr12 | 36236507 | 36236557 |
| chr12 | 75959292 | 75959342 |
| chr12 | 130527959 | 130528009 |
| chr14 | 89681331 | 89681381 |
| chr14 | 89683688 | 89683738 |
| chr15 | 25316700 | 25316750 |
| chr15 | 29139022 | 29139072 |
| chr15 | 41788718 | 41788768 |
| chr15 | 68798226 | 68798276 |
| chr15 | 99950295 | 99950345 |
| chr16 | 59177054 | 59177104 |
| chr17 | 3087684 | 3087734 |
| chr17 | 7877570 | 7877620 |

Table 15CG. Repeat Category MER44D

| | | |
|---|---|---|
| chr17 | 54862299 | 54862349 |
| chr19 | 36679035 | 36679085 |
| chr1 | 151620384 | 151620434 |
| chr1 | 158302000 | 158302050 |
| chr20 | 377409 | 377459 |
| chrX | 19089812 | 19089862 |
| chr2 | 480144 | 480194 |
| chr2 | 5464063 | 5464113 |
| chr2 | 113655772 | 113655822 |
| chr2 | 158731338 | 158731388 |
| chr11 | 47951940 | 47951990 |
| chr3 | 121012704 | 121012754 |
| chr4 | 68337394 | 68337444 |
| chr4 | 68337298 | 68337348 |
| chr4 | 99697671 | 99697721 |
| chr4 | 182162455 | 182162505 |
| chr4 | 182162606 | 182162656 |
| chr5 | 175477133 | 175477183 |
| chr5 | 177555509 | 177555559 |
| chr6 | 158952653 | 158952703 |
| chr7 | 32379009 | 32379059 |
| chr8 | 70739998 | 70740048 |
| chr8 | 122745740 | 122745790 |
| chr9 | 28780615 | 28780665 |
| chr9 | 30012363 | 30012413 |
| chr12 | 49945837 | 49945887 |
| chr16 | 50530109 | 50530159 |
| chr16 | 88404077 | 88404127 |
| chr10 | 80137221 | 80137271 |
| chr10 | 80555868 | 80555918 |

Table 15CH. Repeat Category MER4A1

| | | |
|---|---|---|
| chr17 | 49870706 | 49870756 |
| chr17 | 51481669 | 51481719 |
| chr10 | 86302391 | 86302441 |
| chr10 | 88455635 | 88455685 |
| chr17 | 73317152 | 73317202 |
| chr17 | 75698321 | 75698371 |
| chr17 | 80704216 | 80704266 |
| chr17 | 80811888 | 80811938 |
| chr17 | 80813179 | 80813229 |
| chr18 | 18902000 | 18902050 |
| chr18 | 55496602 | 55496652 |
| chr18 | 72616008 | 72616058 |
| chr19 | 455691 | 455741 |
| chr19 | 9026003 | 9026053 |
| chr19 | 16279737 | 16279787 |
| chr19 | 41612521 | 41612571 |
| chr19 | 43051386 | 43051436 |
| chr10 | 105353082 | 105353132 |
| chr19 | 48640952 | 48641002 |
| chr19 | 56115985 | 56116035 |
| chr19 | 56572072 | 56572122 |
| chr19 | 57988082 | 57988132 |
| chr19 | 63021095 | 63021145 |
| chr19 | 63121278 | 63121328 |
| chr1 | 13188928 | 13188978 |
| chr1 | 22778772 | 22778822 |
| chr1 | 44059871 | 44059921 |
| chr1 | 45184264 | 45184314 |
| chr1 | 66749264 | 66749314 |
| chr1 | 105753294 | 105753344 |
| chr1 | 106420268 | 106420318 |
| chr1 | 114203059 | 114203109 |
| chr10 | 128103409 | 128103459 |
| chr1 | 177743560 | 177743610 |
| chr1 | 199241864 | 199241914 |
| chr1 | 215245164 | 215245214 |
| chr1 | 217947911 | 217947961 |
| chr1 | 217952712 | 217952762 |
| chr1 | 220253922 | 220253972 |
| chr1 | 224010707 | 224010757 |
| chr1 | 226331919 | 226331969 |
| chr1 | 244215327 | 244215377 |
| chr20 | 8745891 | 8745941 |
| chr20 | 25615219 | 25615269 |
| chr20 | 53962566 | 53962616 |
| chr21 | 19045384 | 19045434 |
| chr21 | 19277888 | 19277938 |
| chr21 | 24177822 | 24177872 |
| chr21 | 32453303 | 32453353 |
| chr21 | 43239933 | 43239983 |
| chr21 | 43241599 | 43241649 |
| chr21 | 43244130 | 43244180 |
| chr21 | 45023202 | 45023252 |
| chr22 | 14693356 | 14693406 |
| chr11 | 3097660 | 3097710 |
| chr22 | 46187365 | 46187415 |
| chr22 | 47419707 | 47419757 |
| chr22 | 49126260 | 49126310 |
| chr22 | 49216557 | 49216607 |
| chrX | 2377667 | 2377717 |
| chrX | 5007930 | 5007980 |
| chrX | 22615549 | 22615599 |
| chrX | 27702543 | 27702593 |
| chrX | 31908873 | 31908923 |
| chrX | 42514171 | 42514221 |
| chr11 | 7955709 | 7955759 |
| chrX | 70784660 | 70784710 |
| chrX | 77462256 | 77462306 |
| chrX | 78416582 | 78416632 |
| chrX | 86495599 | 86495649 |
| chrX | 90160577 | 90160627 |
| chrX | 91597227 | 91597277 |
| chrX | 92077828 | 92077878 |
| chrX | 98517246 | 98517296 |
| chrX | 113140416 | 113140466 |
| chrX | 114835369 | 114835419 |
| chrX | 118121177 | 118121227 |
| chrX | 121680850 | 121680900 |
| chrX | 144189263 | 144189313 |
| chrX | 144610443 | 144610493 |
| chrX | 144615032 | 144615082 |
| chrY | 3576941 | 3576991 |
| chrY | 13522685 | 13522735 |
| chrY | 13599141 | 13599191 |
| chrY | 13710408 | 13710458 |
| chrY | 13804461 | 13804511 |
| chr2 | 721783 | 721833 |
| chr2 | 1793728 | 1793778 |
| chr2 | 2898828 | 2898878 |
| chr2 | 2953485 | 2953535 |
| chr2 | 2955905 | 2955955 |
| chr2 | 3041161 | 3041211 |
| chr2 | 8864628 | 8864678 |
| chr2 | 20484867 | 20484917 |
| chr2 | 23811196 | 23811246 |
| chr2 | 25282122 | 25282172 |
| chr2 | 42975071 | 42975121 |
| chr2 | 42976583 | 42976633 |
| chr2 | 84733253 | 84733303 |
| chr2 | 85584112 | 85584162 |
| chr11 | 31283484 | 31283534 |
| chr2 | 108633115 | 108633165 |
| chr2 | 108634777 | 108634827 |
| chr2 | 110194303 | 110194353 |
| chr2 | 113703067 | 113703117 |
| chr2 | 113709135 | 113709185 |
| chr2 | 127792644 | 127792694 |
| chr2 | 131103790 | 131103840 |
| chr2 | 132363106 | 132363156 |
| chr2 | 209211056 | 209211106 |
| chr2 | 235134244 | 235134294 |
| chr2 | 243039826 | 243039876 |
| chr2 | 243044475 | 243044525 |
| chr3 | 16692885 | 16692935 |
| chr3 | 27507094 | 27507144 |
| chr3 | 50309759 | 50309809 |
| chr3 | 53270082 | 53270132 |
| chr3 | 67639578 | 67639628 |
| chr3 | 67639388 | 67639438 |
| chr11 | 58370735 | 58370785 |
| chr3 | 116223969 | 116224019 |
| chr3 | 127305289 | 127305339 |
| chr11 | 60915634 | 60915684 |
| chr10 | 12813826 | 12813876 |
| chr3 | 159666818 | 159666868 |
| chr3 | 163282145 | 163282195 |
| chr3 | 198453535 | 198453585 |
| chr4 | 479935 | 479985 |
| chr4 | 480108 | 480158 |
| chr4 | 482044 | 482094 |
| chr4 | 3095514 | 3095564 |
| chr4 | 5826248 | 5826298 |
| chr4 | 7224898 | 7224948 |
| chr4 | 15453891 | 15453941 |
| chr4 | 20389016 | 20389066 |
| chr4 | 43800982 | 43801032 |
| chr4 | 93405249 | 93405299 |
| chr4 | 93410676 | 93410726 |
| chr4 | 97310234 | 97310284 |
| chr4 | 109273090 | 109273140 |
| chr4 | 141176834 | 141176884 |
| chr4 | 162765339 | 162765389 |
| chr11 | 67216071 | 67216121 |
| chr4 | 166921791 | 166921841 |
| chr4 | 169198637 | 169198687 |
| chr4 | 185942047 | 185942097 |
| chr4 | 189392162 | 189392212 |
| chr5 | 6437024 | 6437074 |
| chr5 | 13573632 | 13573682 |
| chr5 | 21162078 | 21162128 |
| chr5 | 27338460 | 27338510 |
| chr5 | 43032960 | 43033010 |
| chr5 | 43233756 | 43233806 |
| chr5 | 74665846 | 74665896 |
| chr5 | 175838814 | 175838864 |
| chr5 | 180659955 | 180660005 |
| chr6 | 30588169 | 30588219 |
| chr11 | 85372152 | 85372202 |
| chr6 | 63532392 | 63532442 |
| chr6 | 131297278 | 131297328 |
| chr6 | 153761283 | 153761333 |
| chr6 | 167637195 | 167637245 |
| chr6 | 167637718 | 167637768 |
| chr6 | 167637769 | 167637819 |
| chr6 | 169998023 | 169998073 |
| chr7 | 38328301 | 38328351 |
| chr7 | 56043416 | 56043466 |
| chr7 | 63775192 | 63775242 |
| chr7 | 68357469 | 68357519 |
| chr7 | 69894851 | 69894901 |
| chr7 | 70819583 | 70819633 |
| chr7 | 72670749 | 72670799 |
| chr7 | 112819615 | 112819665 |
| chr7 | 139596381 | 139596431 |
| chr7 | 148669600 | 148669650 |
| chr7 | 157648787 | 157648837 |
| chr8 | 3970418 | 3970468 |
| chr8 | 4539613 | 4539663 |
| chr8 | 6724752 | 6724802 |
| chr8 | 6725029 | 6725079 |
| chr8 | 23119513 | 23119563 |
| chr8 | 39966635 | 39966685 |
| chr8 | 59414931 | 59414981 |
| chr8 | 64313726 | 64313776 |
| chr8 | 82456770 | 82456820 |
| chr8 | 120686232 | 120686282 |
| chr8 | 122552822 | 122552872 |
| chr8 | 134204317 | 134204367 |
| chr8 | 143789526 | 143789576 |
| chr9 | 93433855 | 93433905 |
| chr9 | 97152388 | 97152438 |
| chr9 | 108456446 | 108456496 |
| chr12 | 670818 | 670868 |
| chr9 | 128841920 | 128841970 |
| chr12 | 26129464 | 26129514 |
| chr12 | 32056411 | 32056461 |
| chr12 | 38870377 | 38870427 |
| chr12 | 94284872 | 94284922 |
| chr12 | 99558590 | 99558640 |
| chr10 | 36737830 | 36737880 |
| chr13 | 28122798 | 28122848 |
| chr13 | 39644975 | 39645025 |
| chr10 | 38154790 | 38154840 |
| chr13 | 43820304 | 43820354 |
| chr13 | 48366584 | 48366634 |
| chr13 | 60644354 | 60644404 |
| chr13 | 65753901 | 65753951 |
| chr13 | 76494235 | 76494285 |
| chr13 | 97020784 | 97020834 |
| chr13 | 97536085 | 97536135 |
| chr13 | 97537238 | 97537288 |
| chr10 | 43237791 | 43237841 |
| chr14 | 21286271 | 21286321 |
| chr14 | 21662333 | 21662383 |
| chr10 | 50884375 | 50884425 |
| chr14 | 65713847 | 65713897 |
| chr14 | 68196947 | 68196997 |
| chr14 | 68203701 | 68203751 |
| chr14 | 68770504 | 68770554 |
| chr14 | 78347776 | 78347826 |
| chr14 | 88804268 | 88804318 |
| chr14 | 90055443 | 90055493 |
| chr14 | 98456404 | 98456454 |
| chr14 | 99767723 | 99767773 |
| chr14 | 100134095 | 100134145 |
| chr14 | 100135688 | 100135738 |
| chr14 | 100513259 | 100513309 |
| chr14 | 103497584 | 103497634 |
| chr14 | 104410535 | 104410585 |
| chr15 | 23927470 | 23927520 |
| chr15 | 31600999 | 31601049 |
| chr15 | 43478158 | 43478208 |
| chr15 | 49435332 | 49435382 |
| chr15 | 64472013 | 64472063 |
| chr15 | 64477549 | 64477599 |
| chr15 | 68787964 | 68788014 |
| chr15 | 91450470 | 91450520 |
| chr16 | 10377029 | 10377079 |
| chr16 | 12674886 | 12674936 |
| chr16 | 17003180 | 17003230 |
| chr16 | 19932804 | 19932854 |
| chr16 | 30582293 | 30582343 |
| chr16 | 31731702 | 31731752 |
| chr16 | 60010678 | 60010728 |
| chr10 | 3171148 | 3171198 |
| chr16 | 63401241 | 63401291 |
| chr16 | 71856279 | 71856329 |
| chr16 | 74934900 | 74934950 |
| chr16 | 74942548 | 74942598 |
| chr16 | 83774144 | 83774194 |
| chr16 | 83775916 | 83775966 |
| chr16 | 85750104 | 85750154 |
| chr16 | 85750812 | 85750862 |
| chr16 | 87598236 | 87598286 |
| chr10 | 76311073 | 76311123 |
| chr10 | 77922992 | 77923042 |
| chr10 | 3655613 | 3655663 |

Table 15CI. Repeat Category MER4D

| | | |
|---|---|---|
| chr10 | 82621816 | 82621866 |
| chr17 | 49870706 | 49870756 |
| chr10 | 85586493 | 85586543 |
| chr17 | 74958003 | 74958053 |
| chr17 | 77188634 | 77188684 |
| chr17 | 78828710 | 78828760 |
| chr17 | 81391764 | 81391814 |
| chr18 | 646397 | 646447 |
| chr18 | 8209095 | 8209145 |
| chr18 | 8930095 | 8930145 |
| chr18 | 11580210 | 11580260 |
| chr18 | 19487725 | 19487775 |
| chr18 | 25749634 | 25749684 |
| chr18 | 32778894 | 32778944 |
| chr18 | 58501240 | 58501290 |
| chr18 | 60229610 | 60229660 |
| chr18 | 70768005 | 70768055 |
| chr10 | 98986394 | 98986444 |
| chr10 | 99058382 | 99058432 |
| chr19 | 9146080 | 9146130 |
| chr19 | 9773837 | 9773887 |
| chr19 | 11787797 | 11787847 |
| chr19 | 11860077 | 11860127 |
| chr19 | 11897388 | 11897438 |
| chr19 | 11937330 | 11937380 |
| chr19 | 11937572 | 11937622 |
| chr19 | 11962301 | 11962351 |
| chr19 | 12127291 | 12127341 |
| chr19 | 12472046 | 12472096 |
| chr19 | 15045182 | 15045232 |
| chr19 | 40722445 | 40722495 |
| chr10 | 104642279 | 104642329 |
| chr19 | 51372795 | 51372845 |
| chr19 | 56239835 | 56239885 |
| chr19 | 62375436 | 62375486 |
| chr1 | 227716 | 227766 |
| chr1 | 3498101 | 3498151 |
| chr1 | 20034698 | 20034748 |
| chr1 | 32779858 | 32779908 |
| chr1 | 75950522 | 75950572 |
| chr1 | 109129891 | 109129941 |
| chr1 | 171958676 | 171958726 |
| chr1 | 200125039 | 200125089 |
| chr10 | 131670828 | 131670878 |
| chr10 | 131994969 | 131995019 |
| chr1 | 239356917 | 239356967 |
| chr1 | 245993147 | 245993197 |
| chr20 | 3352300 | 3352350 |
| chr20 | 5730321 | 5730371 |
| chr10 | 133573984 | 133574034 |
| chr20 | 35241564 | 35241614 |
| chr20 | 49945522 | 49945572 |
| chr10 | 134140180 | 134140230 |
| chr10 | 134199156 | 134199206 |
| chr20 | 53936656 | 53936706 |
| chr10 | 134251419 | 134251469 |
| chr20 | 62586059 | 62586109 |
| chr20 | 63482161 | 63482211 |
| chr11 | 291673 | 291723 |
| chr11 | 294389 | 294439 |
| chr11 | 296451 | 296501 |
| chr21 | 44491513 | 44491563 |
| chr21 | 44528618 | 44528668 |
| chr21 | 45023202 | 45023252 |
| chr11 | 1749675 | 1749725 |
| chr11 | 1750237 | 1750287 |
| chr11 | 1751395 | 1751445 |
| chr22 | 30892716 | 30892766 |
| chr22 | 37544103 | 37544153 |
| chr11 | 2384001 | 2384051 |
| chr22 | 47304715 | 47304765 |
| chr11 | 3225141 | 3225191 |
| chr11 | 3232339 | 3232389 |
| chrX | 2424897 | 2424947 |
| chrX | 2554743 | 2554793 |
| chrX | 2559458 | 2559508 |
| chrX | 4099109 | 4099159 |
| chr11 | 4144267 | 4144317 |
| chrX | 45457127 | 45457177 |
| chrX | 73201982 | 73202032 |
| chrX | 78723449 | 78723499 |
| chrX | 101035525 | 101035575 |
| chrX | 151195123 | 151195173 |
| chr2 | 9339807 | 9339857 |
| chr2 | 27362976 | 27363026 |
| chr2 | 32271956 | 32272006 |
| chr2 | 39566945 | 39566995 |
| chr2 | 58513645 | 58513695 |
| chr2 | 70394704 | 70394754 |
| chr2 | 71575369 | 71575419 |
| chr2 | 89153971 | 89154021 |
| chr2 | 109342265 | 109342315 |
| chr2 | 113435500 | 113435550 |
| chr2 | 125518807 | 125518857 |
| chr2 | 127343481 | 127343531 |
| chr2 | 129071332 | 129071382 |
| chr2 | 161185602 | 161185652 |
| chr2 | 189496924 | 189496974 |
| chr2 | 202850093 | 202850143 |
| chr2 | 207467114 | 207467164 |
| chr2 | 218277236 | 218277286 |
| chr2 | 229681901 | 229681951 |
| chr2 | 232637365 | 232637415 |
| chr2 | 234096260 | 234096310 |
| chr2 | 242102644 | 242102694 |
| chr3 | 8185018 | 8185068 |
| chr3 | 40477875 | 40477925 |
| chr3 | 40478741 | 40478791 |
| chr3 | 40493720 | 40493770 |
| chr3 | 41966143 | 41966193 |
| chr3 | 41966905 | 41966955 |
| chr3 | 48116258 | 48116308 |
| chr3 | 80803645 | 80803695 |
| chr3 | 97799904 | 97799954 |
| chr3 | 126632907 | 126632957 |
| chr3 | 129700217 | 129700267 |
| chr3 | 130438662 | 130438712 |
| chr3 | 139460807 | 139460857 |
| chr3 | 143908454 | 143908504 |
| chr3 | 150277209 | 150277259 |
| chr11 | 61645572 | 61645622 |
| chr3 | 155873727 | 155873777 |
| chr3 | 180507235 | 180507285 |
| chr3 | 186302236 | 186302286 |
| chr3 | 188059131 | 188059181 |
| chr4 | 8558870 | 8558920 |
| chr4 | 18982233 | 18982283 |
| chr4 | 31858852 | 31858902 |
| chr4 | 57039387 | 57039437 |
| chr4 | 65138773 | 65138823 |
| chr4 | 78463019 | 78463069 |
| chr4 | 85028201 | 85028251 |
| chr11 | 65992489 | 65992539 |
| chr4 | 109566311 | 109566361 |
| chr4 | 115837183 | 115837233 |
| chr4 | 136752614 | 136752664 |
| chr4 | 149022633 | 149022683 |
| chr4 | 152591322 | 152591372 |
| chr4 | 173261016 | 173261066 |
| chr4 | 184573979 | 184574029 |
| chr4 | 188100756 | 188100806 |
| chr5 | 922738 | 922788 |
| chr5 | 1200075 | 1200125 |
| chr5 | 18580782 | 18580832 |
| chr5 | 21133342 | 21133392 |
| chr5 | 79684054 | 79684104 |
| chr5 | 85190623 | 85190673 |
| chr5 | 89922844 | 89922894 |
| chr5 | 104579739 | 104579789 |
| chr5 | 128806264 | 128806314 |
| chr10 | 14696007 | 14696057 |
| chr6 | 15674542 | 15674592 |
| chr11 | 79339197 | 79339247 |
| chr6 | 21826055 | 21826105 |
| chr6 | 26584520 | 26584570 |
| chr6 | 27891793 | 27891843 |
| chr6 | 123889807 | 123889857 |
| chr6 | 136369977 | 136370027 |
| chr6 | 158028345 | 158028395 |
| chr6 | 158518206 | 158518256 |
| chr6 | 167561007 | 167561057 |
| chr7 | 233703 | 233753 |
| chr7 | 12313752 | 12313802 |
| chr7 | 23953975 | 23954025 |
| chr7 | 39501112 | 39501162 |
| chr7 | 44893002 | 44893052 |
| chr7 | 66143928 | 66143978 |
| chr7 | 66396087 | 66396137 |
| chr7 | 66636502 | 66636552 |
| chr7 | 71425048 | 71425098 |
| chr7 | 74728462 | 74728512 |
| chr7 | 100136893 | 100136943 |
| chr7 | 110843993 | 110844043 |
| chr7 | 128177499 | 128177549 |
| chr7 | 137984278 | 137984328 |
| chr11 | 118608749 | 118608799 |
| chr7 | 147615956 | 147616006 |
| chr7 | 156138911 | 156138961 |
| chr7 | 156315764 | 156315814 |
| chr7 | 156314878 | 156314928 |
| chr7 | 158357113 | 158357163 |
| chr8 | 510959 | 511009 |
| chr8 | 1694880 | 1694930 |
| chr8 | 2218028 | 2218078 |
| chr8 | 2217974 | 2218024 |
| chr8 | 11885993 | 11886043 |
| chr8 | 18259074 | 18259124 |
| chr8 | 18258935 | 18258985 |
| chr8 | 41586701 | 41586751 |
| chr11 | 124943555 | 124943605 |
| chr8 | 54723539 | 54723589 |
| chr8 | 75468140 | 75468190 |
| chr8 | 101800524 | 101800574 |
| chr8 | 114484292 | 114484342 |
| chr8 | 120134224 | 120134274 |
| chr8 | 144387871 | 144387921 |
| chr8 | 145333224 | 145333274 |
| chr9 | 37741692 | 37741742 |
| chr9 | 37743608 | 37743658 |
| chr9 | 67095389 | 67095439 |
| chr9 | 69667346 | 69667396 |
| chr9 | 72164587 | 72164637 |
| chr9 | 94422176 | 94422226 |
| chr9 | 96992635 | 96992685 |
| chr9 | 97810507 | 97810557 |
| chr9 | 126222179 | 126222229 |
| chr9 | 132224508 | 132224558 |
| chr9 | 132225400 | 132225450 |
| chr12 | 31923319 | 31923369 |
| chr12 | 55382715 | 55382765 |
| chr12 | 74551747 | 74551797 |
| chr12 | 97385071 | 97385121 |
| chr12 | 101052072 | 101052122 |
| chr12 | 104524039 | 104524089 |
| chr12 | 122983724 | 122983774 |
| chr12 | 126918758 | 126918808 |
| chr12 | 130059965 | 130060015 |
| chr10 | 35238757 | 35238807 |
| chr13 | 34660434 | 34660484 |
| chr13 | 35134322 | 35134372 |
| chr13 | 39464835 | 39464885 |
| chr13 | 111322774 | 111322824 |
| chr13 | 111883466 | 111883516 |
| chr13 | 112387742 | 112387792 |
| chr13 | 112879941 | 112879991 |
| chr14 | 32280572 | 32280622 |
| chr14 | 48009425 | 48009475 |
| chr15 | 87211979 | 87212029 |
| chr16 | 743625 | 743675 |
| chr16 | 767689 | 767739 |
| chr16 | 3769164 | 3769214 |
| chr16 | 10851196 | 10851246 |
| chr16 | 24423044 | 24423094 |
| chr16 | 48322132 | 48322182 |
| chr10 | 397536 | 397586 |
| chr16 | 65767611 | 65767661 |
| chr16 | 68051722 | 68051772 |
| chr16 | 68508183 | 68508233 |
| chr16 | 74804551 | 74804601 |
| chr16 | 84828681 | 84828731 |
| chr16 | 87676103 | 87676153 |
| chr16 | 87676000 | 87676050 |
| chr17 | 7045807 | 7045857 |
| chr17 | 19575870 | 19575920 |
| chr17 | 21175835 | 21175885 |
| chr17 | 26927387 | 26927437 |
| chr10 | 81487637 | 81487687 |

Table 15CJ. Repeat Category MER4D1

| | | |
|---|---|---|
| chr10 | 85586493 | 85586543 |
| chr17 | 77188634 | 77188684 |
| chr17 | 81391764 | 81391814 |
| chr18 | 646397 | 646447 |
| chr18 | 8930095 | 8930145 |
| chr18 | 32778894 | 32778944 |
| chr10 | 99058382 | 99058432 |
| chr19 | 9146080 | 9146130 |
| chr19 | 9773837 | 9773887 |
| chr19 | 15045182 | 15045232 |
| chr19 | 40722445 | 40722495 |
| chr10 | 104642279 | 104642329 |
| chr19 | 62375436 | 62375486 |
| chr1 | 227716 | 227766 |
| chr1 | 3498101 | 3498151 |
| chr1 | 20034698 | 20034748 |
| chr1 | 75950522 | 75950572 |
| chr1 | 109129891 | 109129941 |
| chr1 | 171958676 | 171958726 |
| chr10 | 131994969 | 131995019 |
| chr20 | 3352300 | 3352350 |
| chr20 | 35241564 | 35241614 |
| chr10 | 134140180 | 134140230 |
| chr20 | 53936656 | 53936706 |
| chr10 | 134251419 | 134251469 |
| chr20 | 62586059 | 62586109 |
| chr22 | 30892716 | 30892766 |
| chr11 | 2384001 | 2384051 |
| chr11 | 3225141 | 3225191 |
| chr11 | 3232339 | 3232389 |
| chrX | 2554743 | 2554793 |
| chrX | 2559458 | 2559508 |
| chrX | 78723449 | 78723499 |
| chrX | 151195123 | 151195173 |
| chr2 | 9339807 | 9339857 |
| chr2 | 32271956 | 32272006 |
| chr2 | 39566945 | 39566995 |
| chr2 | 127343481 | 127343531 |
| chr2 | 129071332 | 129071382 |
| chr2 | 161185602 | 161185652 |
| chr2 | 207467114 | 207467164 |
| chr2 | 229681901 | 229681951 |
| chr2 | 234096260 | 234096310 |
| chr2 | 242102644 | 242102694 |
| chr3 | 40477875 | 40477925 |
| chr3 | 40478741 | 40478791 |
| chr3 | 41966143 | 41966193 |
| chr3 | 41966905 | 41966955 |
| chr3 | 48116258 | 48116308 |
| chr3 | 97799904 | 97799954 |
| chr3 | 126632907 | 126632957 |
| chr3 | 130438662 | 130438712 |
| chr3 | 139460807 | 139460857 |
| chr3 | 143908454 | 143908504 |
| chr3 | 150277209 | 150277259 |
| chr3 | 180507235 | 180507285 |
| chr4 | 18982233 | 18982283 |
| chr4 | 31858852 | 31858902 |
| chr4 | 57039387 | 57039437 |
| chr4 | 78463019 | 78463069 |
| chr11 | 65992489 | 65992539 |
| chr4 | 115837183 | 115837233 |
| chr4 | 152591322 | 152591372 |
| chr4 | 173261016 | 173261066 |
| chr5 | 18580782 | 18580832 |
| chr5 | 79684054 | 79684104 |
| chr5 | 85190623 | 85190673 |
| chr10 | 14696007 | 14696057 |
| chr11 | 79339197 | 79339247 |
| chr6 | 21826055 | 21826105 |
| chr6 | 26584520 | 26584570 |
| chr6 | 136369977 | 136370027 |
| chr6 | 158028345 | 158028395 |
| chr6 | 158518206 | 158518256 |
| chr7 | 23953975 | 23954025 |
| chr7 | 66143928 | 66143978 |
| chr7 | 66636502 | 66636552 |
| chr7 | 71425048 | 71425098 |
| chr7 | 100136893 | 100136943 |
| chr11 | 118608749 | 118608799 |
| chr7 | 147615956 | 147616006 |
| chr7 | 156138911 | 156138961 |
| chr7 | 156315764 | 156315814 |
| chr7 | 156314878 | 156314928 |
| chr8 | 1694880 | 1694930 |
| chr11 | 124943555 | 124943605 |
| chr8 | 75468140 | 75468190 |
| chr9 | 67095389 | 67095439 |
| chr9 | 69667346 | 69667396 |
| chr9 | 94422176 | 94422226 |
| chr9 | 126222179 | 126222229 |
| chr12 | 74551747 | 74551797 |
| chr12 | 97385071 | 97385121 |
| chr12 | 101052072 | 101052122 |
| chr12 | 104524039 | 104524089 |
| chr12 | 126918758 | 126918808 |
| chr10 | 35238757 | 35238807 |
| chr13 | 35134322 | 35134372 |
| chr13 | 39464835 | 39464885 |
| chr13 | 112879941 | 112879991 |
| chr14 | 32280572 | 32280622 |
| chr16 | 3769164 | 3769214 |
| chr16 | 10851196 | 10851246 |
| chr10 | 397536 | 397586 |
| chr16 | 68051722 | 68051772 |
| chr16 | 84828681 | 84828731 |
| chr17 | 7045807 | 7045857 |
| chr17 | 19575870 | 19575920 |
| chr17 | 26927387 | 26927437 |
| chr10 | 81487637 | 81487687 |

Table 15CK. Repeat Category MER50

| | | |
|---|---|---|
| chr17 | 40475514 | 40475564 |
| chr17 | 53884816 | 53884866 |
| chr17 | 58107096 | 58107146 |
| chr17 | 62870332 | 62870382 |
| chr10 | 87963270 | 87963320 |
| chr17 | 67083099 | 67083149 |
| chr17 | 67085464 | 67085514 |
| chr17 | 67086764 | 67086814 |
| chr17 | 74975548 | 74975598 |
| chr17 | 77211706 | 77211756 |
| chr18 | 566669 | 566719 |
| chr18 | 11574668 | 11574718 |
| chr18 | 11893485 | 11893535 |
| chr18 | 27621381 | 27621431 |
| chr18 | 47434648 | 47434698 |
| chr10 | 97647539 | 97647589 |
| chr18 | 54624629 | 54624679 |
| chr18 | 56875379 | 56875429 |
| chr18 | 57784078 | 57784128 |
| chr18 | 58260787 | 58260837 |
| chr18 | 66066645 | 66066695 |
| chr18 | 66258530 | 66258580 |
| chr18 | 71804744 | 71804794 |
| chr10 | 5477194 | 5477244 |
| chr19 | 7745018 | 7745068 |
| chr19 | 34823147 | 34823197 |
| chr19 | 35085381 | 35085431 |
| chr10 | 104315655 | 104315705 |
| chr19 | 43056431 | 43056481 |
| chr10 | 5820538 | 5820588 |
| chr10 | 109024049 | 109024099 |
| chr10 | 110812158 | 110812208 |
| chr19 | 53193503 | 53193553 |
| chr10 | 113528085 | 113528135 |
| chr19 | 59041021 | 59041071 |
| chr1 | 953470 | 953520 |
| chr1 | 955570 | 955620 |
| chr1 | 4142427 | 4142477 |
| chr1 | 4142178 | 4142228 |
| chr1 | 5588551 | 5588601 |
| chr1 | 16879489 | 16879539 |
| chr1 | 21181175 | 21181225 |
| chr10 | 122195791 | 122195841 |
| chr10 | 122195339 | 122195389 |
| chr1 | 51443031 | 51443081 |
| chr1 | 75540050 | 75540100 |
| chr1 | 91092173 | 91092223 |
| chr1 | 113878663 | 113878713 |
| chr1 | 166324713 | 166324763 |
| chr1 | 166324524 | 166324574 |
| chr1 | 168634712 | 168634762 |
| chr1 | 170880426 | 170880476 |
| chr1 | 171313647 | 171313697 |
| chr1 | 180102022 | 180102072 |
| chr1 | 181371002 | 181371052 |
| chr1 | 209379148 | 209379198 |
| chr1 | 221770585 | 221770635 |
| chr1 | 226097278 | 226097328 |
| chr1 | 231740697 | 231740747 |
| chr1 | 231880229 | 231880279 |
| chr1 | 231880683 | 231880733 |
| chr1 | 231880831 | 231880881 |
| chr1 | 233162664 | 233162714 |
| chr1 | 238262350 | 238262400 |
| chr1 | 238366933 | 238366983 |
| chr20 | 36868801 | 36868851 |
| chr20 | 37657210 | 37657260 |
| chr20 | 46088016 | 46088066 |
| chr20 | 50559574 | 50559624 |
| chr20 | 53754419 | 53754469 |
| chr20 | 59797353 | 59797403 |
| chr21 | 18019066 | 18019116 |
| chr21 | 20996422 | 20996472 |
| chr21 | 21375745 | 21375795 |
| chr21 | 22873116 | 22873166 |
| chr21 | 24958013 | 24958063 |
| chr21 | 29486042 | 29486092 |
| chr21 | 29486429 | 29486479 |
| chr21 | 36406379 | 36406429 |
| chr21 | 42915781 | 42915831 |
| chr21 | 44493151 | 44493201 |
| chr21 | 44751827 | 44751877 |
| chr22 | 21702098 | 21702148 |
| chr22 | 22389230 | 22389280 |
| chr22 | 22390769 | 22390819 |
| chr22 | 31279946 | 31279996 |
| chr22 | 31281147 | 31281197 |
| chr22 | 34833896 | 34833946 |
| chr10 | 9924602 | 9924652 |
| chr22 | 43536939 | 43536989 |
| chr22 | 43541654 | 43541704 |
| chrX | 4800824 | 4800874 |
| chrX | 8346003 | 8346053 |
| chrX | 14429276 | 14429326 |
| chrX | 15407411 | 15407461 |
| chrX | 46285807 | 46285857 |
| chrX | 86117641 | 86117691 |
| chrX | 86129602 | 86129652 |
| chrX | 86586306 | 86586356 |
| chrX | 88602663 | 88602713 |
| chrX | 96318456 | 96318506 |
| chr11 | 9992783 | 9992833 |
| chrX | 124980125 | 124980175 |
| chrX | 129601377 | 129601427 |
| chrX | 143048066 | 143048116 |
| chrX | 143694000 | 143694050 |
| chrX | 144926632 | 144926682 |
| chrX | 150986787 | 150986837 |
| chrY | 3724548 | 3724598 |
| chr2 | 813473 | 813523 |
| chr2 | 7255905 | 7255955 |
| chr2 | 7259220 | 7259270 |
| chr2 | 10713571 | 10713621 |
| chr2 | 12372624 | 12372674 |
| chr2 | 13036364 | 13036414 |
| chr2 | 29288848 | 29288898 |
| chr2 | 30308884 | 30308934 |
| chr2 | 37626244 | 37626294 |
| chr11 | 20772386 | 20772436 |
| chr11 | 21768694 | 21768744 |
| chr2 | 56542682 | 56542732 |
| chr2 | 61107046 | 61107096 |
| chr2 | 61993416 | 61993466 |
| chr11 | 26088559 | 26088609 |
| chr2 | 72291892 | 72291942 |
| chr2 | 76616916 | 76616966 |
| chr2 | 77982071 | 77982121 |
| chr2 | 101597281 | 101597331 |
| chr2 | 101923598 | 101923648 |
| chr2 | 116700997 | 116701047 |
| chr2 | 121943323 | 121943373 |
| chr2 | 129070564 | 129070614 |
| chr2 | 129071332 | 129071382 |
| chr2 | 138630897 | 138630947 |
| chr2 | 139205273 | 139205323 |
| chr2 | 140648849 | 140648899 |
| chr2 | 169846117 | 169846167 |
| chr2 | 169845855 | 169845905 |
| chr2 | 186562952 | 186563002 |
| chr2 | 194557380 | 194557430 |
| chr2 | 196984680 | 196984730 |
| chr2 | 198439386 | 198439436 |
| chr2 | 201934867 | 201934917 |
| chr2 | 204999799 | 204999849 |
| chr2 | 217182228 | 217182278 |
| chr2 | 228239538 | 228239588 |
| chr2 | 228848729 | 228848779 |
| chr2 | 232507178 | 232507228 |
| chr3 | 9605152 | 9605202 |
| chr3 | 16117645 | 16117695 |
| chr3 | 16233185 | 16233235 |
| chr3 | 19147900 | 19147950 |
| chr3 | 33471139 | 33471189 |
| chr3 | 45319611 | 45319661 |
| chr3 | 47706294 | 47706344 |
| chr3 | 50031815 | 50031865 |
| chr3 | 53904824 | 53904874 |
| chr3 | 57904887 | 57904937 |
| chr3 | 60674238 | 60674288 |
| chr11 | 57407129 | 57407179 |
| chr3 | 100016954 | 100017004 |
| chr3 | 104206050 | 104206100 |
| chr3 | 104206790 | 104206840 |
| chr3 | 105560607 | 105560657 |
| chr3 | 127061100 | 127061150 |
| chr3 | 127613424 | 127613474 |
| chr3 | 134503235 | 134503285 |
| chr3 | 143946141 | 143946191 |
| chr3 | 149458833 | 149458883 |
| chr3 | 163625887 | 163625937 |
| chr3 | 194924619 | 194924669 |
| chr3 | 195969315 | 195969365 |
| chr3 | 196194851 | 196194901 |
| chr3 | 197390941 | 197390991 |
| chr3 | 197391066 | 197391116 |
| chr4 | 5659821 | 5659871 |
| chr4 | 8674719 | 8674769 |
| chr4 | 9707683 | 9707733 |
| chr4 | 15672767 | 15672817 |
| chr4 | 33784887 | 33784937 |
| chr4 | 37953700 | 37953750 |
| chr4 | 57625192 | 57625242 |
| chr4 | 66323064 | 66323114 |
| chr4 | 78469120 | 78469170 |
| chr4 | 79061569 | 79061619 |
| chr4 | 89315394 | 89315444 |
| chr4 | 110105944 | 110105994 |
| chr4 | 115704843 | 115704893 |
| chr4 | 132588188 | 132588238 |
| chr4 | 136007219 | 136007269 |
| chr4 | 146385129 | 146385179 |
| chr4 | 147206181 | 147206231 |
| chr4 | 151666715 | 151666765 |
| chr4 | 156921015 | 156921065 |
| chr4 | 156921119 | 156921169 |
| chr4 | 156921601 | 156921651 |
| chr4 | 156921661 | 156921711 |
| chr4 | 162325233 | 162325283 |
| chr4 | 172360765 | 172360815 |
| chr4 | 173292494 | 173292544 |
| chr4 | 174159601 | 174159651 |
| chr4 | 177261252 | 177261302 |
| chr4 | 186362152 | 186362202 |
| chr4 | 189431979 | 189432029 |
| chr5 | 230617 | 230667 |
| chr5 | 2970627 | 2970677 |
| chr5 | 3059054 | 3059104 |
| chr5 | 13460833 | 13460883 |
| chr5 | 14609839 | 14609889 |
| chr5 | 18589610 | 18589660 |
| chr5 | 23756624 | 23756674 |
| chr5 | 29110961 | 29111011 |
| chr5 | 29110665 | 29110715 |
| chr5 | 38648614 | 38648664 |
| chr5 | 43562065 | 43562115 |
| chr5 | 51680922 | 51680972 |
| chr5 | 55193132 | 55193182 |
| chr5 | 68232572 | 68232622 |
| chr5 | 68497399 | 68497449 |
| chr5 | 68557784 | 68557834 |
| chr5 | 108032405 | 108032455 |
| chr5 | 112022411 | 112022461 |
| chr5 | 114807957 | 114808007 |
| chr5 | 119286423 | 119286473 |
| chr5 | 119447740 | 119447790 |
| chr5 | 126161998 | 126162048 |
| chr5 | 151732275 | 151732325 |
| chr5 | 174954406 | 174954456 |
| chr11 | 76352926 | 76352976 |
| chr6 | 2884781 | 2884831 |
| chr6 | 6231265 | 6231315 |
| chr11 | 77486368 | 77486418 |
| chr6 | 11211938 | 11211988 |
| chr6 | 11212433 | 11212483 |
| chr6 | 11212748 | 11212798 |
| chr6 | 11219727 | 11219777 |
| chr11 | 78032918 | 78032968 |
| chr6 | 15094559 | 15094609 |
| chr6 | 28937722 | 28937772 |
| chr6 | 28937622 | 28937672 |
| chr6 | 28938505 | 28938555 |
| chr11 | 88994865 | 88994915 |
| chr6 | 65387152 | 65387202 |
| chr6 | 66271660 | 66271710 |
| chr6 | 79183274 | 79183324 |
| chr6 | 85387403 | 85387453 |
| chr11 | 92351129 | 92351179 |
| chr6 | 109964713 | 109964763 |
| chr6 | 110860280 | 110860330 |
| chr6 | 111363795 | 111363845 |
| chr6 | 123138363 | 123138413 |
| chr6 | 123279225 | 123279275 |
| chr6 | 131688049 | 131688099 |
| chr6 | 133124888 | 133124938 |
| chr6 | 137603072 | 137603122 |
| chr6 | 151242532 | 151242582 |
| chr6 | 158037804 | 158037854 |
| chr11 | 101878159 | 101878209 |
| chr6 | 164281775 | 164281825 |
| chr6 | 166448032 | 166448082 |
| chr6 | 167888561 | 167888611 |
| chr6 | 167888721 | 167888771 |
| chr6 | 167888898 | 167888948 |
| chr6 | 167889392 | 167889442 |
| chr6 | 167889576 | 167889626 |
| chr6 | 167889858 | 167889908 |
| chr6 | 167890047 | 167890097 |
| chr11 | 107190533 | 107190583 |
| chr7 | 2997245 | 2997295 |
| chr7 | 7427513 | 7427563 |
| chr7 | 7469628 | 7469678 |
| chr7 | 9869259 | 9869309 |
| chr7 | 21173104 | 21173154 |
| chr7 | 21194663 | 21194713 |
| chr7 | 22995847 | 22995897 |
| chr7 | 38472857 | 38472907 |
| chr7 | 39501112 | 39501162 |
| chr7 | 42499293 | 42499343 |
| chr7 | 64859931 | 64859981 |
| chr11 | 114990576 | 114990626 |
| chr7 | 68404656 | 68404706 |
| chr7 | 72226086 | 72226136 |
| chr7 | 77251116 | 77251166 |
| chr7 | 88987751 | 88987801 |
| chr7 | 89575707 | 89575757 |
| chr7 | 100127855 | 100127905 |
| chr7 | 110842571 | 110842621 |
| chr7 | 130457591 | 130457641 |
| chr7 | 133932604 | 133932654 |
| chr7 | 137524888 | 137524938 |
| chr7 | 144873247 | 144873297 |
| chr7 | 149260926 | 149260976 |
| chr7 | 156137421 | 156137471 |
| chr8 | 2339733 | 2339783 |
| chr8 | 3799720 | 3799770 |
| chr8 | 8677276 | 8677326 |
| chr8 | 9084007 | 9084057 |
| chr8 | 10366578 | 10366628 |
| chr8 | 19338318 | 19338368 |
| chr8 | 22957298 | 22957348 |
| chr8 | 22987369 | 22987419 |
| chr8 | 23037501 | 23037551 |
| chr8 | 31396983 | 31397033 |
| chr8 | 58165155 | 58165205 |
| chr8 | 58166699 | 58166749 |
| chr8 | 58168596 | 58168646 |
| chr8 | 58172838 | 58172888 |
| chr8 | 58178500 | 58178550 |
| chr8 | 58183164 | 58183214 |
| chr8 | 62865385 | 62865435 |
| chr8 | 107948675 | 107948725 |
| chr8 | 114494083 | 114494133 |
| chr8 | 115311075 | 115311125 |
| chr8 | 115541567 | 115541617 |
| chr8 | 121579312 | 121579362 |
| chr8 | 125931758 | 125931808 |
| chr8 | 141961852 | 141961902 |
| chr8 | 142520013 | 142520063 |
| chr8 | 146238004 | 146238054 |
| chr10 | 19979247 | 19979297 |
| chr9 | 20273788 | 20273838 |
| chr9 | 21920449 | 21920499 |
| chr9 | 21920252 | 21920302 |
| chr9 | 35478526 | 35478576 |
| chr9 | 110490314 | 110490364 |
| chr9 | 113882503 | 113882553 |
| chr9 | 116459500 | 116459550 |
| chr9 | 124455240 | 124455290 |
| chr12 | 12971558 | 12971608 |
| chr12 | 19733833 | 19733883 |
| chr10 | 23683554 | 23683604 |
| chr12 | 29265161 | 29265211 |
| chr12 | 30113908 | 30113958 |
| chr12 | 30114951 | 30115001 |
| chr12 | 39426722 | 39426772 |
| chr10 | 26727781 | 26727831 |
| chr10 | 27000579 | 27000629 |
| chr12 | 81555801 | 81555851 |
| chr12 | 91978491 | 91978541 |
| chr12 | 94734691 | 94734741 |
| chr10 | 29889872 | 29889922 |
| chr10 | 29890075 | 29890125 |
| chr12 | 103335448 | 103335498 |
| chr12 | 104340511 | 104340561 |
| chr12 | 117582591 | 117582641 |
| chr10 | 32250821 | 32250871 |
| chr12 | 120203636 | 120203686 |
| chr12 | 120596532 | 120596582 |
| chr12 | 122506037 | 122506087 |
| chr12 | 125485678 | 125485728 |
| chr12 | 126249765 | 126249815 |
| chr12 | 128868938 | 128868988 |
| chr13 | 40988367 | 40988417 |
| chr13 | 42080060 | 42080110 |
| chr13 | 68924762 | 68924812 |
| chr13 | 75846298 | 75846348 |
| chr13 | 78804261 | 78804311 |
| chr13 | 86534256 | 86534306 |
| chr14 | 21187554 | 21187604 |
| chr14 | 29698765 | 29698815 |
| chr14 | 30700038 | 30700088 |
| chr14 | 41298867 | 41298917 |
| chr14 | 46653531 | 46653581 |
| chr14 | 51624752 | 51624802 |
| chr14 | 52950024 | 52950074 |
| chr10 | 1769451 | 1769501 |
| chr10 | 1772658 | 1772708 |
| chr14 | 59887314 | 59887364 |
| chr14 | 69970655 | 69970705 |
| chr14 | 72040326 | 72040376 |
| chr14 | 76091376 | 76091426 |
| chr14 | 81104366 | 81104416 |
| chr14 | 85611558 | 85611608 |
| chr10 | 54128381 | 54128431 |
| chr14 | 91028550 | 91028600 |
| chr14 | 94572110 | 94572160 |
| chr14 | 98139119 | 98139169 |
| chr14 | 101573197 | 101573247 |
| chr10 | 2203728 | 2203778 |
| chr14 | 103553155 | 103553205 |
| chr14 | 104385493 | 104385543 |
| chr15 | 20631255 | 20631305 |
| chr15 | 31257720 | 31257770 |
| chr15 | 48820304 | 48820354 |
| chr15 | 48821225 | 48821275 |
| chr10 | 65501634 | 65501684 |
| chr15 | 60227200 | 60227250 |
| chr15 | 79339851 | 79339901 |
| chr15 | 87282589 | 87282639 |
| chr15 | 92797150 | 92797200 |
| chr16 | 1281367 | 1281417 |
| chr16 | 1281875 | 1281925 |
| chr16 | 11364432 | 11364482 |
| chr16 | 13653758 | 13653808 |
| chr16 | 13667863 | 13667913 |
| chr16 | 17544138 | 17544188 |
| chr16 | 21925854 | 21925904 |
| chr16 | 28869672 | 28869722 |
| chr16 | 46750827 | 46750877 |
| chr10 | 73016901 | 73016951 |
| chr16 | 59142958 | 59143008 |
| chr16 | 62261077 | 62261127 |
| chr16 | 63213009 | 63213059 |
| chr16 | 73791391 | 73791441 |
| chr16 | 84775253 | 84775303 |
| chr16 | 86020734 | 86020784 |
| chr17 | 7200812 | 7200862 |
| chr17 | 8304225 | 8304275 |

Table 15CL. Repeat Category MER50B

| | | |
|---|---|---|
| chr17 | 40475514 | 40475564 |
| chr17 | 58107096 | 58107146 |
| chr18 | 11893485 | 11893535 |
| chr18 | 57784078 | 57784128 |
| chr18 | 58260787 | 58260837 |
| chr18 | 66258530 | 66258580 |
| chr10 | 110812158 | 110812208 |
| chr10 | 113528085 | 113528135 |
| chr1 | 168634712 | 168634762 |
| chr1 | 231880229 | 231880279 |
| chr1 | 231880683 | 231880733 |
| chr1 | 231880831 | 231880881 |
| chr20 | 50559574 | 50559624 |
| chr21 | 20996422 | 20996472 |
| chr21 | 29486042 | 29486092 |
| chr21 | 29486429 | 29486479 |
| chr21 | 42915781 | 42915831 |
| chr22 | 21702098 | 21702148 |
| chr22 | 22389230 | 22389280 |
| chr22 | 22390769 | 22390819 |
| chr10 | 9924602 | 9924652 |
| chrX | 8346003 | 8346053 |
| chrX | 15407411 | 15407461 |
| chrX | 86117641 | 86117691 |
| chrX | 96318456 | 96318506 |
| chrX | 124980125 | 124980175 |
| chrX | 129601377 | 129601427 |
| chrX | 143048066 | 143048116 |
| chrX | 144926632 | 144926682 |
| chrX | 150986787 | 150986837 |
| chr11 | 21768694 | 21768744 |
| chr11 | 26088559 | 26088609 |
| chr2 | 72291892 | 72291942 |
| chr2 | 76616916 | 76616966 |
| chr2 | 121943323 | 121943373 |
| chr2 | 129070564 | 129070614 |
| chr2 | 129071332 | 129071382 |
| chr2 | 140648849 | 140648899 |
| chr2 | 186562952 | 186563002 |
| chr2 | 204999799 | 204999849 |
| chr3 | 16117645 | 16117695 |
| chr3 | 19147900 | 19147950 |
| chr3 | 33471139 | 33471189 |
| chr3 | 53904824 | 53904874 |
| chr4 | 15672767 | 15672817 |
| chr4 | 115704843 | 115704893 |
| chr5 | 230617 | 230667 |
| chr5 | 29110961 | 29111011 |
| chr5 | 29110665 | 29110715 |
| chr5 | 38648614 | 38648664 |
| chr5 | 43562065 | 43562115 |
| chr5 | 68557784 | 68557834 |
| chr5 | 119286423 | 119286473 |
| chr5 | 126161998 | 126162048 |
| chr11 | 76352926 | 76352976 |
| chr11 | 88994865 | 88994915 |
| chr6 | 65387152 | 65387202 |
| chr6 | 79183274 | 79183324 |
| chr6 | 110860280 | 110860330 |
| chr6 | 123279225 | 123279275 |
| chr6 | 133124888 | 133124938 |
| chr6 | 158037804 | 158037854 |
| chr6 | 167888561 | 167888611 |
| chr6 | 167888721 | 167888771 |
| chr6 | 167888898 | 167888948 |
| chr6 | 167889392 | 167889442 |
| chr6 | 167889576 | 167889626 |
| chr6 | 167889858 | 167889908 |
| chr6 | 167890047 | 167890097 |
| chr7 | 7427513 | 7427563 |
| chr7 | 68404656 | 68404706 |
| chr7 | 88987751 | 88987801 |
| chr7 | 89575707 | 89575757 |
| chr7 | 110842571 | 110842621 |
| chr7 | 133932604 | 133932654 |
| chr7 | 144873247 | 144873297 |
| chr7 | 149260926 | 149260976 |
| chr8 | 3799720 | 3799770 |
| chr8 | 9084007 | 9084057 |
| chr8 | 19338318 | 19338368 |
| chr8 | 22987369 | 22987419 |
| chr8 | 107948675 | 107948725 |
| chr8 | 115311075 | 115311125 |
| chr9 | 20273788 | 20273838 |
| chr9 | 35478526 | 35478576 |
| chr9 | 113882503 | 113882553 |
| chr9 | 116459500 | 116459550 |
| chr10 | 29889872 | 29889922 |
| chr10 | 29890075 | 29890125 |
| chr13 | 68924762 | 68924812 |
| chr13 | 78804261 | 78804311 |
| chr13 | 86534256 | 86534306 |
| chr14 | 41298867 | 41298917 |
| chr14 | 52950024 | 52950074 |
| chr14 | 72040326 | 72040376 |
| chr10 | 2203728 | 2203778 |
| chr16 | 13653758 | 13653808 |
| chr16 | 13667863 | 13667913 |
| chr16 | 21925854 | 21925904 |
| chr16 | 46750827 | 46750877 |
| chr16 | 62261077 | 62261127 |
| chr17 | 7200812 | 7200862 |

Table 15CM. Repeat Category MER51A

| | | |
|---|---|---|
| chr18 | 13128507 | 13128557 |
| chr18 | 13451298 | 13451348 |
| chr18 | 13451194 | 13451244 |
| chr18 | 54644218 | 54644268 |
| chr19 | 23641507 | 23641557 |
| chr19 | 33631977 | 33632027 |
| chr19 | 33636370 | 33636420 |
| chr19 | 39584959 | 39585009 |
| chr19 | 45041301 | 45041351 |
| chr19 | 63460502 | 63460552 |
| chr1 | 3441400 | 3441450 |
| chr1 | 9788735 | 9788785 |
| chr1 | 88825390 | 88825440 |
| chr1 | 92979420 | 92979470 |
| chr1 | 95080303 | 95080353 |
| chr1 | 152837627 | 152837677 |
| chr1 | 197122950 | 197123000 |
| chr1 | 226814532 | 226814582 |
| chr20 | 50528292 | 50528342 |
| chr20 | 62139297 | 62139347 |
| chr20 | 62140316 | 62140366 |
| chr21 | 37858415 | 37858465 |
| chr21 | 42085048 | 42085098 |
| chr21 | 46129945 | 46129995 |
| chr22 | 27454517 | 27454567 |
| chr22 | 45504901 | 45504951 |
| chrX | 2440265 | 2440315 |
| chrX | 16153038 | 16153088 |
| chrX | 17756325 | 17756375 |
| chrX | 39859197 | 39859247 |
| chrX | 39923048 | 39923098 |
| chrX | 117075723 | 117075773 |
| chrX | 117251510 | 117251560 |
| chrX | 117858647 | 117858697 |
| chrX | 129566370 | 129566420 |
| chr2 | 44472360 | 44472410 |
| chr2 | 117121876 | 117121926 |
| chr11 | 33165743 | 33165793 |
| chr2 | 137292398 | 137292448 |
| chr3 | 44753207 | 44753257 |
| chr3 | 49584281 | 49584331 |
| chr11 | 58213235 | 58213285 |
| chr11 | 58787567 | 58787617 |
| chr3 | 130976233 | 130976283 |
| chr3 | 186294700 | 186294750 |
| chr4 | 3715489 | 3715539 |
| chr4 | 42097523 | 42097573 |
| chr4 | 89833227 | 89833277 |
| chr4 | 155265272 | 155265322 |
| chr5 | 70967020 | 70967070 |
| chr5 | 70967098 | 70967148 |
| chr5 | 72508920 | 72508970 |
| chr5 | 150406150 | 150406200 |
| chr5 | 150658336 | 150658386 |
| chr5 | 154972859 | 154972909 |
| chr5 | 166992531 | 166992581 |
| chr11 | 76280215 | 76280265 |
| chr11 | 77094352 | 77094402 |
| chr6 | 24776747 | 24776797 |
| chr10 | 15280948 | 15280998 |
| chr6 | 53272990 | 53273040 |
| chr6 | 104939867 | 104939917 |
| chr6 | 105318610 | 105318660 |
| chr11 | 94073642 | 94073692 |
| chr7 | 43073657 | 43073707 |
| chr7 | 73512196 | 73512246 |
| chr7 | 107854039 | 107854089 |
| chr7 | 111992638 | 111992688 |
| chr8 | 31092964 | 31093014 |
| chr11 | 125450297 | 125450347 |
| chr8 | 82814839 | 82814889 |
| chr11 | 133253322 | 133253372 |
| chr9 | 35478526 | 35478576 |
| chr9 | 37458208 | 37458258 |
| chr9 | 37458995 | 37459045 |
| chr9 | 108340671 | 108340721 |
| chr9 | 128841920 | 128841970 |
| chr12 | 6880388 | 6880438 |
| chr12 | 12794253 | 12794303 |
| chr12 | 32009471 | 32009521 |
| chr12 | 32710607 | 32710657 |
| chr12 | 50259195 | 50259245 |
| chr12 | 86149346 | 86149396 |
| chr12 | 100959205 | 100959255 |
| chr13 | 18493512 | 18493562 |
| chr13 | 75228906 | 75228956 |
| chr14 | 51191313 | 51191363 |
| chr14 | 51293738 | 51293788 |
| chr14 | 59145480 | 59145530 |
| chr15 | 38590889 | 38590939 |
| chr15 | 62835162 | 62835212 |
| chr15 | 67917232 | 67917282 |
| chr16 | 1629987 | 1630037 |
| chr16 | 3518969 | 3519019 |
| chr16 | 3518839 | 3518889 |
| chr16 | 8159255 | 8159305 |
| chr16 | 49798300 | 49798350 |
| chr16 | 78716343 | 78716393 |
| chr17 | 15237562 | 15237612 |
| chr17 | 29989357 | 29989407 |
| chr17 | 31393734 | 31393784 |
| chr17 | 31396394 | 31396444 |
| chr17 | 31399124 | 31399174 |
| chr17 | 33905512 | 33905562 |

Table 15CN. Repeat Category MER51B

| | | |
|---|---|---|
| chr18 | 42621314 | 42621364 |
| chr18 | 42621070 | 42621120 |
| chr18 | 46175076 | 46175126 |
| chr19 | 9046086 | 9046136 |
| chr1 | 27117829 | 27117879 |
| chr1 | 46839910 | 46839960 |
| chr1 | 46839776 | 46839826 |
| chr1 | 52676846 | 52676896 |
| chr1 | 80003542 | 80003592 |
| chr1 | 80006532 | 80006582 |
| chr1 | 91139029 | 91139079 |
| chr1 | 152493527 | 152493577 |
| chr1 | 163711434 | 163711484 |
| chr1 | 207821617 | 207821667 |
| chr1 | 239696896 | 239696946 |
| chr10 | 133256240 | 133256290 |
| chr10 | 133261722 | 133261772 |
| chr20 | 36274397 | 36274447 |
| chr10 | 134660820 | 134660870 |
| chr21 | 45869408 | 45869458 |
| chr21 | 46129945 | 46129995 |
| chrX | 75974047 | 75974097 |
| chrX | 109550892 | 109550942 |
| chrX | 118910669 | 118910719 |
| chrX | 118916368 | 118916418 |
| chrX | 121653168 | 121653218 |
| chr2 | 137292398 | 137292448 |
| chr2 | 196504924 | 196504974 |
| chr2 | 237879687 | 237879737 |
| chr11 | 57168685 | 57168735 |
| chr11 | 58787567 | 58787617 |
| chr3 | 122994234 | 122994284 |
| chr3 | 134835210 | 134835260 |
| chr3 | 170852608 | 170852658 |
| chr10 | 12923712 | 12923762 |
| chr4 | 123132 | 123182 |
| chr4 | 122867 | 122917 |
| chr4 | 24394633 | 24394683 |
| chr4 | 25343454 | 25343504 |
| chr4 | 84513306 | 84513356 |
| chr4 | 115934383 | 115934433 |
| chr4 | 140733946 | 140733996 |
| chr4 | 140735296 | 140735346 |
| chr4 | 140737199 | 140737249 |
| chr4 | 140739007 | 140739057 |
| chr4 | 140740735 | 140740785 |
| chr4 | 187712639 | 187712689 |
| chr4 | 187712729 | 187712779 |
| chr5 | 33711979 | 33712029 |
| chr5 | 43227812 | 43227862 |
| chr5 | 43230241 | 43230291 |
| chr11 | 70901543 | 70901593 |
| chr5 | 150650151 | 150650201 |
| chr6 | 39767599 | 39767649 |
| chr6 | 42965775 | 42965825 |
| chr6 | 42967989 | 42968039 |
| chr6 | 42968703 | 42968753 |
| chr7 | 1356073 | 1356123 |
| chr7 | 47916601 | 47916651 |
| chr7 | 70826814 | 70826864 |
| chr7 | 80790080 | 80790130 |
| chr7 | 105534829 | 105534879 |
| chr7 | 151800784 | 151800834 |
| chr7 | 152043172 | 152043222 |
| chr8 | 26993015 | 26993065 |
| chr8 | 101781290 | 101781340 |
| chr8 | 121503949 | 121503999 |
| chr9 | 134945018 | 134945068 |
| chr9 | 135017098 | 135017148 |
| chr9 | 135017151 | 135017201 |
| chr10 | 22571035 | 22571085 |
| chr12 | 61140321 | 61140371 |
| chr13 | 18128959 | 18129009 |
| chr13 | 18130048 | 18130098 |
| chr13 | 67555939 | 67555989 |
| chr14 | 25679839 | 25679889 |
| chr14 | 32901443 | 32901493 |
| chr14 | 45041736 | 45041786 |
| chr14 | 45045484 | 45045534 |
| chr14 | 101703348 | 101703398 |
| chr15 | 39153821 | 39153871 |
| chr15 | 70692488 | 70692538 |
| chr10 | 3048073 | 3048123 |
| chr16 | 10826739 | 10826789 |
| chr16 | 46464711 | 46464761 |
| chr16 | 46464632 | 46464682 |
| chr16 | 46465470 | 46465520 |
| chr16 | 46634350 | 46634400 |
| chr10 | 73088875 | 73088925 |
| chr16 | 87598236 | 87598286 |

Table 15CO. Repeat Category MER57A-int

| | | |
|---|---|---|
| chr17 | 71650885 | 71650935 |
| chr10 | 89054688 | 89054738 |
| chr18 | 51633492 | 51633542 |
| chr18 | 51633214 | 51633264 |
| chr19 | 19794057 | 19794107 |
| chr19 | 19832922 | 19832972 |
| chr19 | 20447698 | 20447748 |
| chr19 | 20571559 | 20571609 |
| chr19 | 21985933 | 21985983 |
| chr19 | 22190440 | 22190490 |
| chr19 | 22802989 | 22803039 |
| chr19 | 23087435 | 23087485 |
| chr19 | 23086715 | 23086765 |
| chr19 | 23370300 | 23370350 |
| chr19 | 23372156 | 23372206 |
| chr19 | 23764904 | 23764954 |
| chr19 | 47777169 | 47777219 |
| chr19 | 47777386 | 47777436 |
| chr19 | 47778183 | 47778233 |
| chr19 | 48133627 | 48133677 |
| chr19 | 48258991 | 48259041 |
| chr19 | 48261399 | 48261449 |
| chr10 | 107096851 | 107096901 |
| chr19 | 50570168 | 50570218 |
| chr1 | 77060879 | 77060929 |
| chr1 | 81528340 | 81528390 |
| chr1 | 150913487 | 150913537 |
| chr1 | 158114298 | 158114348 |
| chr1 | 173115742 | 173115792 |
| chr1 | 179933430 | 179933480 |
| chr1 | 202276010 | 202276060 |
| chr1 | 210268275 | 210268325 |
| chr1 | 225800182 | 225800232 |
| chr1 | 225802953 | 225803003 |
| chr20 | 24195235 | 24195285 |
| chr20 | 30581384 | 30581434 |
| chr20 | 33451278 | 33451328 |
| chr20 | 42803404 | 42803454 |
| chr20 | 47180930 | 47180980 |
| chr10 | 134829903 | 134829953 |
| chr21 | 32852866 | 32852916 |
| chr22 | 15704972 | 15705022 |
| chr11 | 1883069 | 1883119 |
| chr11 | 1887412 | 1887462 |
| chr11 | 1889686 | 1889736 |
| chrX | 26135420 | 26135470 |
| chrX | 88202876 | 88202926 |
| chrX | 89435851 | 89435901 |
| chrX | 125771444 | 125771494 |
| chrY | 3316496 | 3316546 |
| chr2 | 69655094 | 69655144 |
| chr2 | 85693928 | 85693978 |
| chr2 | 207041579 | 207041629 |
| chr2 | 219857800 | 219857850 |
| chr2 | 236491263 | 236491313 |
| chr3 | 33469000 | 33469050 |
| chr3 | 126459975 | 126460025 |
| chr3 | 130413623 | 130413673 |
| chr3 | 151890279 | 151890329 |
| chr3 | 164275494 | 164275544 |
| chr3 | 169600014 | 169600064 |
| chr4 | 72182187 | 72182237 |
| chr4 | 124175490 | 124175540 |
| chr5 | 37805368 | 37805418 |
| chr5 | 102458904 | 102458954 |
| chr5 | 110297575 | 110297625 |
| chr5 | 110304838 | 110304888 |
| chr5 | 152137696 | 152137746 |
| chr5 | 171704461 | 171704511 |
| chr5 | 171706637 | 171706687 |
| chr5 | 174965181 | 174965231 |
| chr5 | 174966747 | 174966797 |
| chr5 | 174975174 | 174975224 |
| chr5 | 175786623 | 175786673 |
| chr5 | 175787006 | 175787056 |
| chr6 | 33152778 | 33152828 |
| chr6 | 85004326 | 85004376 |
| chr6 | 132832115 | 132832165 |
| chr6 | 137679565 | 137679615 |
| chr6 | 142403775 | 142403825 |
| chr6 | 145498033 | 145498083 |
| chr6 | 153761283 | 153761333 |
| chr7 | 13549564 | 13549614 |
| chr7 | 32722959 | 32723009 |
| chr7 | 32953863 | 32953913 |
| chr7 | 32955588 | 32955638 |
| chr7 | 55442030 | 55442080 |
| chr7 | 64095746 | 64095796 |
| chr7 | 64243980 | 64244030 |
| chr7 | 96559666 | 96559716 |
| chr7 | 96561265 | 96561315 |
| chr7 | 103231387 | 103231437 |
| chr7 | 158525233 | 158525283 |
| chr8 | 18070627 | 18070677 |
| chr11 | 124965214 | 124965264 |
| chr8 | 66835944 | 66835994 |
| chr8 | 74879262 | 74879312 |
| chr8 | 103954219 | 103954269 |
| chr8 | 125328895 | 125328945 |
| chr8 | 125332371 | 125332421 |
| chr8 | 144870176 | 144870226 |
| chr9 | 72531694 | 72531744 |
| chr9 | 72532974 | 72533024 |
| chr9 | 129840359 | 129840409 |
| chr9 | 129841253 | 129841303 |
| chr10 | 260703 | 260753 |
| chr12 | 25996105 | 25996155 |
| chr12 | 29569903 | 29569953 |
| chr12 | 29569724 | 29569774 |
| chr12 | 34244293 | 34244343 |
| chr13 | 36798213 | 36798263 |
| chr10 | 42151059 | 42151109 |
| chr10 | 46447419 | 46447469 |
| chr14 | 30226176 | 30226226 |
| chr14 | 36681991 | 36682041 |
| chr14 | 36683226 | 36683276 |
| chr10 | 48936967 | 48937017 |
| chr14 | 69334788 | 69334838 |
| chr14 | 83370269 | 83370319 |
| chr14 | 85575100 | 85575150 |
| chr15 | 23888365 | 23888415 |
| chr15 | 91039462 | 91039512 |
| chr16 | 2780566 | 2780616 |
| chr16 | 27965328 | 27965378 |
| chr16 | 74373896 | 74373946 |
| chr17 | 14516084 | 14516134 |
| chr17 | 14518717 | 14518767 |

Table 15CP. Repeat Category MER6

| | | |
|---|---|---|
| chr17 | 38344004 | 38344054 |
| chr10 | 81859646 | 81859696 |
| chr10 | 81859236 | 81859286 |
| chr10 | 81987068 | 81987118 |
| chr10 | 81996443 | 81996493 |
| chr10 | 81996779 | 81996829 |
| chr10 | 81996902 | 81996952 |
| chr17 | 42159421 | 42159471 |
| chr17 | 42338242 | 42338292 |
| chr17 | 42610400 | 42610450 |
| chr17 | 42806831 | 42806881 |
| chr10 | 82378155 | 82378205 |
| chr17 | 44792287 | 44792337 |
| chr17 | 46041043 | 46041093 |
| chr17 | 46294369 | 46294419 |
| chr17 | 47504094 | 47504144 |
| chr17 | 47945390 | 47945440 |
| chr17 | 48146243 | 48146293 |
| chr10 | 83919766 | 83919816 |
| chr17 | 48326788 | 48326838 |
| chr17 | 48606392 | 48606442 |
| chr17 | 49841124 | 49841174 |
| chr17 | 50076663 | 50076713 |
| chr17 | 50076545 | 50076595 |
| chr17 | 51379827 | 51379877 |
| chr17 | 51474424 | 51474474 |
| chr17 | 53982663 | 53982713 |
| chr17 | 55371841 | 55371891 |
| chr17 | 55383852 | 55383902 |
| chr10 | 85805403 | 85805453 |
| chr10 | 85805945 | 85805995 |
| chr10 | 85949723 | 85949773 |
| chr17 | 59595918 | 59595968 |
| chr17 | 65148923 | 65148973 |
| chr17 | 71139590 | 71139640 |
| chr17 | 71270125 | 71270175 |
| chr17 | 72848106 | 72848156 |
| chr10 | 88647234 | 88647284 |
| chr17 | 73801340 | 73801390 |
| chr17 | 74953388 | 74953438 |
| chr17 | 75176876 | 75176926 |
| chr17 | 75380250 | 75380300 |
| chr17 | 75886905 | 75886955 |
| chr17 | 75888082 | 75888132 |
| chr17 | 76150864 | 76150914 |
| chr17 | 76385061 | 76385111 |
| chr17 | 77036236 | 77036286 |
| chr17 | 77036010 | 77036060 |
| chr17 | 77209288 | 77209338 |
| chr17 | 77265523 | 77265573 |
| chr17 | 77982432 | 77982482 |
| chr17 | 78525870 | 78525920 |
| chr17 | 78527383 | 78527433 |
| chr17 | 78847153 | 78847203 |
| chr17 | 78912489 | 78912539 |
| chr17 | 78912378 | 78912428 |
| chr10 | 91502212 | 91502262 |
| chr17 | 79277553 | 79277603 |
| chr17 | 79459896 | 79459946 |
| chr17 | 79617097 | 79617147 |
| chr17 | 79912643 | 79912693 |
| chr10 | 92494429 | 92494479 |
| chr17 | 80633910 | 80633960 |
| chr17 | 80671667 | 80671717 |
| chr10 | 4545080 | 4545130 |
| chr18 | 3647883 | 3647933 |
| chr18 | 4891166 | 4891216 |
| chr18 | 6275226 | 6275276 |
| chr18 | 8659822 | 8659872 |
| chr18 | 8843767 | 8843817 |
| chr18 | 9425093 | 9425143 |
| chr18 | 10916207 | 10916257 |
| chr18 | 12397116 | 12397166 |
| chr18 | 13561912 | 13561962 |
| chr10 | 94757308 | 94757358 |
| chr18 | 15326824 | 15326874 |
| chr18 | 18708053 | 18708103 |
| chr18 | 24452956 | 24453006 |
| chr18 | 25891941 | 25891991 |
| chr18 | 26200976 | 26201026 |
| chr18 | 26823808 | 26823858 |
| chr18 | 27385061 | 27385111 |
| chr18 | 27660595 | 27660645 |
| chr18 | 27998944 | 27998994 |
| chr18 | 29395873 | 29395923 |
| chr18 | 35631797 | 35631847 |
| chr18 | 41932712 | 41932762 |
| chr18 | 42621314 | 42621364 |
| chr18 | 42621070 | 42621120 |
| chr18 | 42958118 | 42958168 |
| chr10 | 5129359 | 5129409 |
| chr18 | 46554807 | 46554857 |
| chr18 | 50165618 | 50165668 |
| chr18 | 54338110 | 54338160 |
| chr18 | 54819342 | 54819392 |
| chr18 | 55125069 | 55125119 |
| chr18 | 55339400 | 55339450 |
| chr18 | 56314587 | 56314637 |
| chr18 | 56665314 | 56665364 |
| chr18 | 58471019 | 58471069 |
| chr18 | 58690757 | 58690807 |
| chr18 | 58938483 | 58938533 |
| chr18 | 59367070 | 59367120 |
| chr18 | 60142566 | 60142616 |
| chr18 | 62321171 | 62321221 |
| chr18 | 63005517 | 63005567 |
| chr18 | 64670156 | 64670206 |
| chr18 | 65443448 | 65443498 |
| chr18 | 66772560 | 66772610 |
| chr18 | 67314825 | 67314875 |
| chr18 | 67600759 | 67600809 |
| chr18 | 68508443 | 68508493 |
| chr18 | 68510276 | 68510326 |
| chr18 | 69857096 | 69857146 |
| chr18 | 70206558 | 70206608 |
| chr18 | 72581730 | 72581780 |
| chr18 | 72587569 | 72587619 |
| chr18 | 72895069 | 72895119 |
| chr18 | 72894895 | 72894945 |
| chr18 | 73759407 | 73759457 |
| chr18 | 73783026 | 73783076 |
| chr18 | 74579864 | 74579914 |
| chr18 | 75775885 | 75775935 |
| chr18 | 75847633 | 75847683 |
| chr18 | 75847987 | 75848037 |
| chr18 | 76004501 | 76004551 |
| chr18 | 76005154 | 76005204 |
| chr19 | 330893 | 330943 |
| chr19 | 438082 | 438132 |
| chr10 | 99838254 | 99838304 |
| chr10 | 99838182 | 99838232 |
| chr10 | 100049969 | 100050019 |
| chr19 | 2075531 | 2075581 |
| chr19 | 2799091 | 2799141 |
| chr19 | 2896558 | 2896608 |
| chr19 | 2918744 | 2918794 |
| chr19 | 3150092 | 3150142 |
| chr19 | 4085549 | 4085599 |
| chr19 | 4209367 | 4209417 |
| chr19 | 4595931 | 4595981 |
| chr19 | 4704468 | 4704518 |
| chr19 | 4738227 | 4738277 |
| chr19 | 5727319 | 5727369 |
| chr19 | 6756116 | 6756166 |
| chr19 | 7079091 | 7079141 |
| chr19 | 7135546 | 7135596 |
| chr19 | 7721357 | 7721407 |
| chr19 | 7722794 | 7722844 |
| chr19 | 7917845 | 7917895 |
| chr19 | 8969662 | 8969712 |
| chr19 | 9030711 | 9030761 |
| chr19 | 9032426 | 9032476 |
| chr19 | 9135767 | 9135817 |
| chr19 | 11748558 | 11748608 |
| chr19 | 11913797 | 11913847 |
| chr19 | 12045258 | 12045308 |
| chr19 | 12237854 | 12237904 |
| chr19 | 13630328 | 13630378 |
| chr19 | 13653674 | 13653724 |
| chr19 | 14292747 | 14292797 |
| chr10 | 102974097 | 102974147 |
| chr19 | 15033996 | 15034046 |
| chr19 | 16580710 | 16580760 |
| chr19 | 16581183 | 16581233 |
| chr19 | 18036599 | 18036649 |
| chr19 | 18156781 | 18156831 |
| chr19 | 20057352 | 20057402 |
| chr19 | 20262196 | 20262246 |
| chr19 | 20352774 | 20352824 |
| chr19 | 22272329 | 22272379 |
| chr19 | 22399725 | 22399775 |
| chr19 | 22559892 | 22559942 |
| chr19 | 22616724 | 22616774 |
| chr19 | 37744604 | 37744654 |
| chr19 | 37756027 | 37756077 |
| chr19 | 38213021 | 38213071 |
| chr19 | 39024517 | 39024567 |
| chr19 | 39324871 | 39324921 |
| chr19 | 39419557 | 39419607 |
| chr19 | 39801207 | 39801257 |
| chr19 | 39807847 | 39807897 |
| chr19 | 40384870 | 40384920 |
| chr19 | 42870509 | 42870559 |
| chr19 | 42871851 | 42871901 |
| chr19 | 42872894 | 42872944 |
| chr19 | 42878521 | 42878571 |
| chr19 | 43752065 | 43752115 |
| chr19 | 43909308 | 43909358 |
| chr19 | 44206013 | 44206063 |
| chr19 | 44214716 | 44214766 |
| chr19 | 44215126 | 44215176 |
| chr19 | 44216030 | 44216080 |
| chr19 | 44216311 | 44216361 |
| chr19 | 44216779 | 44216829 |
| chr19 | 44216859 | 44216909 |
| chr19 | 44220917 | 44220967 |
| chr19 | 44226379 | 44226429 |
| chr19 | 44229185 | 44229235 |
| chr19 | 44312811 | 44312861 |
| chr19 | 45313274 | 45313324 |
| chr19 | 45365521 | 45365571 |
| chr19 | 45365588 | 45365638 |
| chr19 | 46924570 | 46924620 |
| chr19 | 46925002 | 46925052 |
| chr19 | 46925274 | 46925324 |
| chr19 | 46925554 | 46925604 |
| chr19 | 46925864 | 46925914 |
| chr19 | 46964552 | 46964602 |
| chr19 | 46964978 | 46965028 |
| chr19 | 46965165 | 46965215 |
| chr19 | 46965363 | 46965413 |
| chr19 | 47813530 | 47813580 |
| chr19 | 47866984 | 47867034 |
| chr10 | 106643402 | 106643452 |
| chr19 | 48189349 | 48189399 |
| chr19 | 48540615 | 48540665 |
| chr19 | 48540321 | 48540371 |
| chr19 | 48654504 | 48654554 |
| chr19 | 49386611 | 49386661 |
| chr19 | 50148168 | 50148218 |
| chr19 | 50363451 | 50363501 |
| chr19 | 50363638 | 50363688 |
| chr19 | 50507087 | 50507137 |
| chr19 | 50507255 | 50507305 |
| chr10 | 109025149 | 109025199 |
| chr19 | 51679736 | 51679786 |
| chr19 | 51680044 | 51680094 |
| chr19 | 51680181 | 51680231 |
| chr19 | 52459396 | 52459446 |
| chr19 | 52459695 | 52459745 |
| chr19 | 52485290 | 52485340 |
| chr19 | 52540881 | 52540931 |
| chr19 | 53450073 | 53450123 |
| chr19 | 54499741 | 54499791 |
| chr19 | 55191494 | 55191544 |
| chr19 | 55636124 | 55636174 |
| chr19 | 56094980 | 56095030 |
| chr19 | 56503997 | 56504047 |
| chr19 | 56549951 | 56550001 |
| chr19 | 56556545 | 56556595 |
| chr19 | 56672956 | 56673006 |
| chr19 | 57834593 | 57834643 |
| chr19 | 57921962 | 57922012 |
| chr19 | 57933409 | 57933459 |
| chr19 | 58054452 | 58054502 |
| chr19 | 58139364 | 58139414 |
| chr19 | 58192274 | 58192324 |
| chr19 | 58551674 | 58551724 |
| chr19 | 59022879 | 59022929 |
| chr19 | 59520855 | 59520905 |
| chr19 | 59521103 | 59521153 |
| chr19 | 59791392 | 59791442 |
| chr10 | 6059521 | 6059571 |
| chr19 | 60145473 | 60145523 |
| chr10 | 114275315 | 114275365 |
| chr10 | 114275170 | 114275220 |
| chr19 | 60931278 | 60931328 |
| chr19 | 61172002 | 61172052 |
| chr19 | 61369087 | 61369137 |
| chr19 | 61673912 | 61673962 |
| chr19 | 61673765 | 61673815 |
| chr19 | 62206915 | 62206965 |
| chr19 | 63624475 | 63624525 |
| chr1 | 1219130 | 1219180 |
| chr1 | 1225629 | 1225679 |
| chr1 | 1463120 | 1463170 |
| chr1 | 1959049 | 1959099 |
| chr1 | 2222080 | 2222130 |
| chr10 | 115289131 | 115289181 |
| chr1 | 3958458 | 3958508 |
| chr1 | 4390316 | 4390366 |
| chr10 | 115879925 | 115879975 |
| chr1 | 8853924 | 8853974 |
| chr1 | 8886972 | 8887022 |
| chr1 | 9179315 | 9179365 |
| chr10 | 117046557 | 117046607 |
| chr1 | 9297577 | 9297627 |
| chr1 | 10696981 | 10697031 |
| chr1 | 11042462 | 11042512 |
| chr1 | 11395225 | 11395275 |
| chr1 | 13900746 | 13900796 |
| chr1 | 14530795 | 14530845 |
| chr1 | 17395065 | 17395115 |
| chr1 | 19584604 | 19584654 |
| chr1 | 19693410 | 19693460 |
| chr1 | 21945039 | 21945089 |
| chr10 | 119668846 | 119668896 |
| chr1 | 22727204 | 22727254 |
| chr1 | 23062260 | 23062310 |
| chr1 | 24033094 | 24033144 |
| chr1 | 24064272 | 24064322 |
| chr1 | 26953633 | 26953683 |
| chr1 | 30150643 | 30150693 |
| chr1 | 32282590 | 32282640 |
| chr1 | 32605274 | 32605324 |
| chr1 | 36098518 | 36098568 |
| chr1 | 37416193 | 37416243 |
| chr1 | 39126439 | 39126489 |
| chr1 | 39601783 | 39601833 |
| chr1 | 39798956 | 39799006 |
| chr1 | 39879966 | 39880016 |
| chr1 | 43212208 | 43212258 |
| chr1 | 43248074 | 43248124 |
| chr1 | 43933310 | 43933360 |
| chr1 | 43937538 | 43937588 |
| chr1 | 45456542 | 45456592 |
| chr1 | 45776159 | 45776209 |
| chr1 | 46160842 | 46160892 |
| chr1 | 46858225 | 46858275 |
| chr1 | 49623259 | 49623309 |
| chr1 | 50188781 | 50188831 |
| chr1 | 52065869 | 52065919 |
| chr1 | 57666817 | 57666867 |
| chr1 | 61572991 | 61573041 |
| chr1 | 62160833 | 62160883 |
| chr1 | 62563329 | 62563379 |
| chr1 | 62868674 | 62868724 |
| chr1 | 63031228 | 63031278 |
| chr1 | 66911631 | 66911681 |
| chr10 | 6581662 | 6581712 |
| chr1 | 69048209 | 69048259 |
| chr10 | 124286273 | 124286323 |
| chr1 | 75928156 | 75928206 |
| chr1 | 79318898 | 79318948 |
| chr1 | 81768927 | 81768977 |
| chr1 | 81769070 | 81769120 |
| chr1 | 81769354 | 81769404 |
| chr1 | 82665820 | 82665870 |
| chr1 | 86088966 | 86089016 |
| chr1 | 86089283 | 86089333 |
| chr1 | 89101819 | 89101869 |
| chr1 | 90821623 | 90821673 |
| chr1 | 101254657 | 101254707 |
| chr1 | 101549591 | 101549641 |
| chr1 | 106534127 | 106534177 |
| chr1 | 107441342 | 107441392 |
| chr10 | 126161462 | 126161512 |
| chr1 | 113035091 | 113035141 |
| chr1 | 113615663 | 113615713 |
| chr1 | 114743350 | 114743400 |
| chr1 | 115767146 | 115767196 |
| chr1 | 116751098 | 116751148 |
| chr1 | 119827966 | 119828016 |
| chr1 | 144545295 | 144545345 |
| chr1 | 146301886 | 146301936 |
| chr10 | 126558072 | 126558122 |
| chr1 | 147248224 | 147248274 |
| chr1 | 147394506 | 147394556 |
| chr1 | 147965385 | 147965435 |
| chr1 | 149137045 | 149137095 |
| chr1 | 149149284 | 149149334 |
| chr1 | 149875317 | 149875367 |
| chr1 | 149875082 | 149875132 |
| chr10 | 127063354 | 127063404 |
| chr1 | 150894801 | 150894851 |
| chr10 | 127124121 | 127124171 |
| chr1 | 151439811 | 151439861 |
| chr1 | 153351320 | 153351370 |
| chr1 | 153416440 | 153416490 |
| chr1 | 154089424 | 154089474 |
| chr1 | 164732488 | 164732538 |
| chr1 | 168781942 | 168781992 |
| chr1 | 168867429 | 168867479 |
| chr1 | 172605308 | 172605358 |
| chr1 | 173925499 | 173925549 |
| chr1 | 176913658 | 176913708 |
| chr1 | 185754121 | 185754171 |
| chr1 | 187276631 | 187276681 |
| chr1 | 187341491 | 187341541 |
| chr1 | 191824637 | 191824687 |
| chr1 | 192481463 | 192481513 |
| chr1 | 192740517 | 192740567 |
| chr1 | 193433524 | 193433574 |
| chr1 | 197896466 | 197896516 |
| chr10 | 130052346 | 130052396 |
| chr1 | 200863454 | 200863504 |
| chr1 | 201033088 | 201033138 |
| chr1 | 201200664 | 201200714 |
| chr1 | 201953707 | 201953757 |
| chr1 | 201953594 | 201953644 |
| chr1 | 206210941 | 206210991 |
| chr1 | 207597559 | 207597609 |
| chr1 | 208377402 | 208377452 |
| chr1 | 208587073 | 208587123 |
| chr1 | 208689514 | 208689564 |
| chr1 | 209940709 | 209940759 |
| chr1 | 217611296 | 217611346 |
| chr1 | 217977905 | 217977955 |
| chr1 | 221305685 | 221305735 |
| chr1 | 221305737 | 221305787 |
| chr1 | 223777431 | 223777481 |
| chr1 | 223778863 | 223778913 |
| chr1 | 224597813 | 224597863 |
| chr1 | 225272829 | 225272879 |
| chr1 | 225283938 | 225283988 |
| chr1 | 225283678 | 225283728 |
| chr1 | 225293282 | 225293332 |
| chr1 | 225300517 | 225300567 |
| chr1 | 225382647 | 225382697 |
| chr1 | 225747345 | 225747395 |
| chr1 | 225747174 | 225747224 |
| chr1 | 227900599 | 227900649 |
| chr1 | 228032360 | 228032410 |
| chr1 | 228456118 | 228456168 |
| chr1 | 228550874 | 228550924 |
| chr1 | 230959049 | 230959099 |
| chr1 | 231333978 | 231334028 |
| chr1 | 232610909 | 232610959 |
| chr1 | 232729835 | 232729885 |
| chr1 | 233736058 | 233736108 |
| chr1 | 233736976 | 233737026 |
| chr1 | 235473221 | 235473271 |
| chr1 | 235690014 | 235690064 |
| chr1 | 235689725 | 235689775 |
| chr1 | 236442530 | 236442580 |
| chr10 | 131844589 | 131844639 |
| chr1 | 239052461 | 239052511 |
| chr1 | 239920809 | 239920859 |
| chr1 | 239986913 | 239986963 |
| chr1 | 241661772 | 241661822 |
| chr1 | 241803821 | 241803871 |
| chr1 | 241809257 | 241809307 |
| chr1 | 241846302 | 241846352 |
| chr1 | 242390069 | 242390119 |
| chr1 | 242390126 | 242390176 |
| chr1 | 243818801 | 243818851 |
| chr1 | 245891792 | 245891842 |
| chr1 | 245935745 | 245935795 |
| chr1 | 245936730 | 245936780 |
| chr20 | 1639037 | 1639087 |
| chr20 | 2422840 | 2422890 |
| chr20 | 4175265 | 4175315 |
| chr20 | 4794544 | 4794594 |
| chr20 | 6039693 | 6039743 |
| chr20 | 8352707 | 8352757 |
| chr20 | 8785045 | 8785095 |
| chr20 | 11841858 | 11841908 |
| chr20 | 12744038 | 12744088 |
| chr20 | 15219245 | 15219295 |
| chr20 | 16051417 | 16051467 |
| chr20 | 17886196 | 17886246 |
| chr20 | 18167373 | 18167423 |
| chr20 | 19256468 | 19256518 |
| chr20 | 20243404 | 20243454 |
| chr20 | 23335972 | 23336022 |
| chr20 | 23422199 | 23422249 |
| chr20 | 25016578 | 25016628 |
| chr10 | 133428372 | 133428422 |
| chr20 | 30581384 | 30581434 |
| chr20 | 30751197 | 30751247 |
| chr20 | 32753740 | 32753790 |
| chr20 | 33853027 | 33853077 |
| chr20 | 33981949 | 33981999 |
| chr20 | 37358484 | 37358534 |
| chr20 | 37942315 | 37942365 |
| chr20 | 42661734 | 42661784 |
| chr20 | 42758827 | 42758877 |
| chr20 | 44181864 | 44181914 |
| chr20 | 44664779 | 44664829 |
| chr20 | 45782257 | 45782307 |
| chr20 | 49260463 | 49260513 |
| chr20 | 49475854 | 49475904 |
| chr10 | 134096423 | 134096473 |
| chr20 | 50477256 | 50477306 |
| chr20 | 51279747 | 51279797 |
| chr20 | 51896460 | 51896510 |
| chr20 | 51901415 | 51901465 |
| chr20 | 53137589 | 53137639 |
| chr20 | 53138979 | 53139029 |
| chr20 | 55773201 | 55773251 |
| chr20 | 56722814 | 56722864 |
| chr20 | 58741009 | 58741059 |
| chr10 | 134482282 | 134482332 |
| chr10 | 134484047 | 134484097 |
| chr20 | 61697029 | 61697079 |
| chr20 | 62138028 | 62138078 |
| chr20 | 62214453 | 62214503 |
| chr21 | 13527355 | 13527405 |
| chr21 | 16298966 | 16299016 |
| chr21 | 18205559 | 18205609 |
| chr21 | 18713696 | 18713746 |
| chr21 | 19282559 | 19282609 |
| chr11 | 296451 | 296501 |
| chr21 | 27325006 | 27325056 |
| chr21 | 28219920 | 28219970 |
| chr21 | 28795879 | 28795929 |
| chr21 | 30498133 | 30498183 |
| chr21 | 31486787 | 31486837 |
| chr21 | 31772150 | 31772200 |
| chr11 | 503059 | 503109 |
| chr21 | 34454130 | 34454180 |
| chr21 | 34703931 | 34703981 |
| chr21 | 34703667 | 34703717 |
| chr21 | 35314962 | 35315012 |
| chr21 | 35314819 | 35314869 |
| chr21 | 36357715 | 36357765 |
| chr21 | 37224689 | 37224739 |
| chr21 | 39948282 | 39948332 |
| chr21 | 39948080 | 39948130 |
| chr21 | 39948173 | 39948223 |
| chr21 | 40375173 | 40375223 |
| chr21 | 40909024 | 40909074 |
| chr21 | 41477600 | 41477650 |
| chr21 | 41477372 | 41477422 |
| chr21 | 41654572 | 41654622 |
| chr21 | 41654818 | 41654868 |
| chr21 | 41936038 | 41936088 |
| chr21 | 42380336 | 42380386 |
| chr21 | 42380123 | 42380173 |
| chr21 | 42394531 | 42394581 |
| chr21 | 42499339 | 42499389 |
| chr21 | 43111213 | 43111263 |
| chr21 | 43454585 | 43454635 |
| chr21 | 43579489 | 43579539 |
| chr21 | 43580417 | 43580467 |
| chr21 | 43620913 | 43620963 |
| chr21 | 44476192 | 44476242 |
| chr21 | 44878855 | 44878905 |
| chr21 | 44928959 | 44929009 |
| chr21 | 45172222 | 45172272 |
| chr21 | 45172062 | 45172112 |
| chr21 | 45308514 | 45308564 |
| chr21 | 45554593 | 45554643 |
| chr21 | 45644766 | 45644816 |
| chr21 | 46405158 | 46405208 |
| chr21 | 46698938 | 46698988 |
| chr22 | 15279166 | 15279216 |
| chr22 | 16032014 | 16032064 |
| chr22 | 16838133 | 16838183 |
| chr22 | 16838034 | 16838084 |
| chr22 | 17946807 | 17946857 |
| chr22 | 18306254 | 18306304 |
| chr22 | 18308023 | 18308073 |
| chr22 | 18313807 | 18313857 |
| chr22 | 20691028 | 20691078 |
| chr22 | 21248399 | 21248449 |
| chr22 | 23843183 | 23843233 |
| chr22 | 23950631 | 23950681 |
| chr22 | 23968411 | 23968461 |
| chr22 | 27485279 | 27485329 |
| chr22 | 30222092 | 30222142 |
| chr22 | 32092206 | 32092256 |
| chr22 | 32094548 | 32094598 |
| chr11 | 2006284 | 2006334 |
| chr22 | 33952404 | 33952454 |
| chr22 | 33974246 | 33974296 |
| chr22 | 34118405 | 34118455 |
| chr22 | 34212852 | 34212902 |
| chr22 | 34945205 | 34945255 |
| chr22 | 36399697 | 36399747 |
| chr22 | 37402223 | 37402273 |
| chr22 | 37693764 | 37693814 |
| chr22 | 37945312 | 37945362 |
| chr22 | 38350185 | 38350235 |
| chr22 | 38438682 | 38438732 |
| chr10 | 9924602 | 9924652 |
| chr22 | 40442172 | 40442222 |
| chr22 | 40445175 | 40445225 |
| chr22 | 41890927 | 41890977 |
| chr22 | 44216198 | 44216248 |
| chr22 | 45222376 | 45222426 |
| chr22 | 45222121 | 45222171 |
| chr22 | 45290559 | 45290609 |
| chr22 | 45600005 | 45600055 |
| chr22 | 45600670 | 45600720 |
| chr22 | 46175915 | 46175965 |
| chr22 | 46175829 | 46175879 |
| chr22 | 46581440 | 46581490 |
| chr22 | 47274931 | 47274981 |
| chr22 | 47706854 | 47706904 |
| chr22 | 48149617 | 48149667 |
| chr11 | 3419617 | 3419667 |
| chr11 | 3420732 | 3420782 |
| chr22 | 48519005 | 48519055 |
| chr22 | 48695800 | 48695850 |
| chr22 | 48865760 | 48865810 |
| chr22 | 48958556 | 48958606 |
| chr22 | 49106701 | 49106751 |
| chrX | 3103984 | 3104034 |
| chr11 | 3994157 | 3994207 |
| chrX | 11555492 | 11555542 |
| chrX | 12178927 | 12178977 |
| chrX | 12178763 | 12178813 |
| chrX | 13301473 | 13301523 |
| chr11 | 4274291 | 4274341 |
| chrX | 14880461 | 14880511 |
| chrX | 18220299 | 18220349 |
| chrX | 18759297 | 18759347 |
| chrX | 20920809 | 20920859 |
| chr11 | 5410204 | 5410254 |
| chrX | 23329497 | 23329547 |
| chrX | 24011092 | 24011142 |
| chrX | 24013248 | 24013298 |
| chrX | 26823249 | 26823299 |
| chrX | 28582994 | 28583044 |
| chrX | 30468831 | 30468881 |
| chr11 | 6298286 | 6298336 |
| chrX | 33851654 | 33851704 |
| chrX | 36198961 | 36199011 |
| chrX | 36444161 | 36444211 |
| chrX | 38684803 | 38684853 |
| chrX | 41682982 | 41683032 |
| chrX | 42067191 | 42067241 |
| chrX | 42067067 | 42067117 |
| chrX | 43096256 | 43096306 |
| chrX | 46410325 | 46410375 |
| chrX | 46465327 | 46465377 |
| chrX | 46767981 | 46768031 |
| chrX | 47112601 | 47112651 |
| chrX | 47512052 | 47512102 |
| chrX | 47892128 | 47892178 |
| chrX | 48978516 | 48978566 |
| chrX | 49641527 | 49641577 |
| chrX | 50271543 | 50271593 |
| chrX | 51019934 | 51019984 |
| chrX | 51800013 | 51800063 |
| chrX | 51816777 | 51816827 |
| chrX | 52440167 | 52440217 |
| chrX | 52453656 | 52453706 |
| chr11 | 8044274 | 8044324 |
| chr11 | 8044169 | 8044219 |
| chrX | 53298965 | 53299015 |
| chrX | 54640989 | 54641039 |
| chrX | 56927997 | 56928047 |
| chrX | 61925053 | 61925103 |
| chrX | 66415982 | 66416032 |
| chrX | 68459317 | 68459367 |
| chrX | 68529915 | 68529965 |
| chrX | 70352118 | 70352168 |
| chrX | 80044639 | 80044689 |
| chrX | 85755076 | 85755126 |
| chrX | 86890711 | 86890761 |
| chrX | 89135662 | 89135712 |
| chrX | 91489211 | 91489261 |
| chrX | 91628640 | 91628690 |
| chrX | 94402626 | 94402676 |
| chrX | 94431177 | 94431227 |
| chrX | 94539704 | 94539754 |
| chrX | 94669760 | 94669810 |
| chrX | 94689115 | 94689165 |
| chrX | 96407080 | 96407130 |
| chrX | 99434415 | 99434465 |
| chr11 | 9758708 | 9758758 |
| chrX | 108601103 | 108601153 |
| chrX | 116832124 | 116832174 |
| chrX | 117392119 | 117392169 |
| chrX | 117393347 | 117393397 |
| chr11 | 10468092 | 10468142 |
| chr11 | 10467949 | 10467999 |
| chrX | 118833010 | 118833060 |
| chrX | 119059210 | 119059260 |
| chrX | 120059532 | 120059582 |
| chrX | 121777001 | 121777051 |
| chrX | 123625197 | 123625247 |
| chr11 | 10775367 | 10775417 |
| chrX | 124460430 | 124460480 |
| chrX | 127443677 | 127443727 |
| chrX | 131626045 | 131626095 |
| chrX | 133702475 | 133702525 |
| chrX | 134605305 | 134605355 |
| chrX | 134789452 | 134789502 |
| chrX | 138749052 | 138749102 |
| chrX | 143188096 | 143188146 |
| chrX | 147345673 | 147345723 |
| chrX | 147589546 | 147589596 |
| chrX | 150586068 | 150586118 |
| chrX | 150855912 | 150855962 |
| chrX | 150866042 | 150866092 |
| chrX | 151545850 | 151545900 |
| chrX | 151545792 | 151545842 |
| chrX | 152195137 | 152195187 |
| chrX | 152678366 | 152678416 |
| chrY | 3274581 | 3274631 |
| chrY | 6176528 | 6176578 |
| chrY | 7876146 | 7876196 |
| chrY | 17607035 | 17607085 |
| chr2 | 721783 | 721833 |
| chr2 | 2083196 | 2083246 |
| chr2 | 2083087 | 2083137 |
| chr2 | 3145169 | 3145219 |
| chr2 | 4416961 | 4417011 |
| chr2 | 8708283 | 8708333 |
| chr2 | 9363992 | 9364042 |
| chr2 | 9534966 | 9535016 |
| chr11 | 17260512 | 17260562 |
| chr2 | 11250782 | 11250832 |
| chr2 | 11896840 | 11896890 |
| chr2 | 13234301 | 13234351 |
| chr2 | 16393967 | 16394017 |
| chr2 | 18449196 | 18449246 |
| chr2 | 21963665 | 21963715 |
| chr11 | 18094249 | 18094299 |
| chr2 | 24157335 | 24157385 |
| chr2 | 28539646 | 28539696 |
| chr2 | 32250060 | 32250110 |
| chr2 | 32252011 | 32252061 |
| chr2 | 34330027 | 34330077 |
| chr2 | 34333508 | 34333558 |
| chr2 | 38332582 | 38332632 |
| chr2 | 39593484 | 39593534 |
| chr2 | 41198646 | 41198696 |
| chr2 | 47897667 | 47897717 |
| chr11 | 21730809 | 21730859 |
| chr2 | 52641697 | 52641747 |
| chr2 | 53966566 | 53966616 |
| chr2 | 53966387 | 53966437 |
| chr2 | 54881521 | 54881571 |
| chr2 | 54888298 | 54888348 |
| chr2 | 56141084 | 56141134 |
| chr11 | 22817421 | 22817471 |
| chr2 | 61059735 | 61059785 |
| chr2 | 61079326 | 61079376 |
| chr2 | 61522111 | 61522161 |
| chr2 | 61742370 | 61742420 |
| chr2 | 61902549 | 61902599 |
| chr2 | 64108613 | 64108663 |
| chr2 | 65267889 | 65267939 |
| chr2 | 73909042 | 73909092 |
| chr2 | 73923028 | 73923078 |
| chr2 | 75032818 | 75032868 |
| chr2 | 75913248 | 75913298 |
| chr2 | 75914672 | 75914722 |
| chr2 | 76646860 | 76646910 |
| chr2 | 77900691 | 77900741 |
| chr2 | 78874346 | 78874396 |
| chr2 | 78878620 | 78878670 |
| chr2 | 80220066 | 80220116 |
| chr2 | 85159349 | 85159399 |
| chr2 | 85451994 | 85452044 |
| chr2 | 86160451 | 86160501 |
| chr2 | 86548260 | 86548310 |
| chr2 | 87368705 | 87368755 |
| chr2 | 90025987 | 90026037 |
| chr2 | 91321660 | 91321710 |
| chr2 | 91551068 | 91551118 |
| chr11 | 28567205 | 28567255 |
| chr2 | 94875133 | 94875183 |
| chr2 | 96585806 | 96585856 |
| chr2 | 97206283 | 97206333 |
| chr2 | 97905579 | 97905629 |
| chr2 | 97944141 | 97944191 |
| chr2 | 98235747 | 98235797 |
| chr2 | 101677777 | 101677827 |
| chr2 | 101778175 | 101778225 |
| chr2 | 106830104 | 106830154 |
| chr2 | 107773049 | 107773099 |
| chr2 | 107772966 | 107773016 |
| chr2 | 108439140 | 108439190 |
| chr2 | 112651090 | 112651140 |
| chr2 | 113553922 | 113553972 |
| chr2 | 115200059 | 115200109 |
| chr10 | 11930284 | 11930334 |
| chr2 | 121729282 | 121729332 |
| chr2 | 122681427 | 122681477 |
| chr2 | 124259716 | 124259766 |
| chr2 | 126001901 | 126001951 |
| chr2 | 127420107 | 127420157 |
| chr2 | 129760002 | 129760052 |
| chr2 | 130830586 | 130830636 |
| chr2 | 132067385 | 132067435 |
| chr2 | 132110033 | 132110083 |
| chr2 | 132332374 | 132332424 |
| chr2 | 133138575 | 133138625 |
| chr2 | 135408846 | 135408896 |
| chr2 | 135642594 | 135642644 |
| chr2 | 136063678 | 136063728 |
| chr2 | 143474129 | 143474179 |
| chr2 | 151600316 | 151600366 |
| chr2 | 154568410 | 154568460 |
| chr2 | 157209241 | 157209291 |
| chr2 | 158255464 | 158255514 |
| chr2 | 164149831 | 164149881 |
| chr2 | 164151287 | 164151337 |
| chr2 | 166687797 | 166687847 |
| chr2 | 171492085 | 171492135 |
| chr2 | 174111817 | 174111867 |
| chr2 | 174361547 | 174361597 |
| chr11 | 36526546 | 36526596 |
| chr2 | 178988419 | 178988469 |
| chr2 | 179046776 | 179046826 |
| chr2 | 180737244 | 180737294 |
| chr2 | 181500026 | 181500076 |
| chr2 | 187397091 | 187397141 |
| chr2 | 188021552 | 188021602 |
| chr2 | 188901929 | 188901979 |
| chr2 | 188943352 | 188943402 |
| chr2 | 194568693 | 194568743 |
| chr2 | 196762315 | 196762365 |
| chr2 | 197192251 | 197192301 |
| chr2 | 197701075 | 197701125 |
| chr11 | 39455224 | 39455274 |
| chr2 | 199045946 | 199045996 |
| chr11 | 39662792 | 39662842 |
| chr2 | 200868561 | 200868611 |
| chr2 | 201364123 | 201364173 |
| chr2 | 202265224 | 202265274 |
| chr2 | 203041019 | 203041069 |
| chr2 | 203095986 | 203096036 |
| chr2 | 203516005 | 203516055 |
| chr11 | 40409480 | 40409530 |
| chr2 | 206690687 | 206690737 |
| chr2 | 207330731 | 207330781 |
| chr2 | 207331706 | 207331756 |
| chr2 | 207618146 | 207618196 |
| chr2 | 208866270 | 208866320 |
| chr2 | 212561548 | 212561598 |
| chr2 | 219120353 | 219120403 |
| chr2 | 220465513 | 220465563 |
| chr2 | 220752896 | 220752946 |
| chr2 | 229551575 | 229551625 |
| chr2 | 230717019 | 230717069 |
| chr2 | 232444962 | 232445012 |
| chr2 | 232924839 | 232924889 |
| chr2 | 232943331 | 232943381 |
| chr2 | 233413519 | 233413569 |
| chr2 | 233413431 | 233413481 |
| chr2 | 236793626 | 236793676 |
| chr2 | 237388877 | 237388927 |
| chr2 | 237753762 | 237753812 |
| chr2 | 237753591 | 237753641 |
| chr2 | 238728083 | 238728133 |
| chr2 | 238858390 | 238858440 |
| chr2 | 240417573 | 240417623 |
| chr2 | 240638557 | 240638607 |
| chr2 | 240988857 | 240988907 |
| chr2 | 241969920 | 241969970 |
| chr2 | 242113098 | 242113148 |
| chr11 | 46409574 | 46409624 |
| chr3 | 2241422 | 2241472 |
| chr3 | 5078949 | 5078999 |
| chr3 | 5079989 | 5080039 |
| chr3 | 10197906 | 10197956 |
| chr10 | 12367846 | 12367896 |
| chr3 | 13468527 | 13468577 |
| chr3 | 13491612 | 13491662 |
| chr3 | 13528671 | 13528721 |
| chr3 | 14627750 | 14627800 |
| chr3 | 14981541 | 14981591 |
| chr3 | 15394903 | 15394953 |
| chr3 | 19251241 | 19251291 |
| chr3 | 21559420 | 21559470 |
| chr3 | 24005272 | 24005322 |
| chr3 | 27480084 | 27480134 |
| chr3 | 27481248 | 27481298 |
| chr3 | 29437246 | 29437296 |
| chr3 | 30108131 | 30108181 |
| chr3 | 32768192 | 32768242 |
| chr3 | 33485601 | 33485651 |
| chr3 | 33490986 | 33491036 |
| chr3 | 33972706 | 33972756 |
| chr3 | 35786197 | 35786247 |
| chr3 | 35786085 | 35786135 |
| chr3 | 36827118 | 36827168 |
| chr3 | 39456485 | 39456535 |
| chr3 | 39458357 | 39458407 |
| chr3 | 39528768 | 39528818 |
| chr3 | 39528612 | 39528662 |
| chr3 | 42450826 | 42450876 |
| chr3 | 43073401 | 43073451 |
| chr3 | 43072935 | 43072985 |
| chr3 | 44687659 | 44687709 |
| chr10 | 12483056 | 12483106 |
| chr11 | 55228251 | 55228301 |
| chr11 | 55746988 | 55747038 |
| chr11 | 55749298 | 55749348 |
| chr3 | 51772761 | 51772811 |
| chr3 | 52753970 | 52754020 |
| chr3 | 52763104 | 52763154 |
| chr3 | 53172979 | 53173029 |
| chr3 | 55338444 | 55338494 |
| chr3 | 60718822 | 60718872 |
| chr3 | 60853704 | 60853754 |
| chr3 | 62349113 | 62349163 |
| chr3 | 65768491 | 65768541 |
| chr3 | 67063196 | 67063246 |
| chr3 | 70462822 | 70462872 |
| chr3 | 75414990 | 75415040 |
| chr11 | 58217662 | 58217712 |
| chr3 | 81957206 | 81957256 |
| chr3 | 85438381 | 85438431 |
| chr3 | 89905854 | 89905904 |
| chr3 | 96404726 | 96404776 |
| chr3 | 97252892 | 97252942 |
| chr3 | 101374368 | 101374418 |
| chr3 | 102658141 | 102658191 |
| chr3 | 106495358 | 106495408 |
| chr3 | 109297348 | 109297398 |
| chr3 | 110060170 | 110060220 |
| chr3 | 110381634 | 110381684 |
| chr3 | 113397077 | 113397127 |
| chr3 | 114479562 | 114479612 |
| chr3 | 114617897 | 114617947 |
| chr3 | 117467151 | 117467201 |
| chr3 | 121500602 | 121500652 |
| chr3 | 121633722 | 121633772 |
| chr3 | 122601051 | 122601101 |
| chr11 | 60582669 | 60582719 |
| chr3 | 126944775 | 126944825 |
| chr3 | 128126644 | 128126694 |
| chr3 | 130110962 | 130111012 |
| chr3 | 131340630 | 131340680 |
| chr3 | 133026334 | 133026384 |
| chr3 | 133324564 | 133324614 |
| chr3 | 133399293 | 133399343 |
| chr3 | 134306500 | 134306550 |
| chr3 | 137735584 | 137735634 |
| chr3 | 137735396 | 137735446 |
| chr3 | 137961827 | 137961877 |
| chr3 | 141768396 | 141768446 |
| chr3 | 142352724 | 142352774 |
| chr3 | 142677472 | 142677522 |
| chr3 | 147960623 | 147960673 |
| chr3 | 154745129 | 154745179 |
| chr3 | 156851531 | 156851581 |
| chr3 | 161111645 | 161111695 |
| chr3 | 161214134 | 161214184 |
| chr3 | 164953738 | 164953788 |
| chr3 | 165027497 | 165027547 |
| chr3 | 172832364 | 172832414 |
| chr3 | 173238168 | 173238218 |
| chr3 | 173833773 | 173833823 |
| chr3 | 175744173 | 175744223 |
| chr3 | 176933788 | 176933838 |
| chr3 | 177826688 | 177826738 |
| chr3 | 181172359 | 181172409 |
| chr3 | 181937467 | 181937517 |
| chr10 | 13003536 | 13003586 |
| chr3 | 186759221 | 186759271 |
| chr3 | 190263915 | 190263965 |
| chr3 | 191546590 | 191546640 |
| chr3 | 191592445 | 191592495 |
| chr3 | 191615997 | 191616047 |
| chr3 | 193102415 | 193102465 |
| chr3 | 195486668 | 195486718 |
| chr4 | 269034 | 269084 |
| chr4 | 378714 | 378764 |
| chr4 | 632936 | 632986 |
| chr4 | 1811455 | 1811505 |
| chr4 | 3013029 | 3013079 |
| chr4 | 3347791 | 3347841 |
| chr4 | 3380234 | 3380284 |
| chr4 | 3580854 | 3580904 |
| chr4 | 4222064 | 4222114 |
| chr4 | 4405851 | 4405901 |
| chr4 | 5591448 | 5591498 |
| chr4 | 5591361 | 5591411 |
| chr4 | 5629895 | 5629945 |
| chr4 | 5744178 | 5744228 |
| chr4 | 5744025 | 5744075 |
| chr4 | 5977763 | 5977813 |
| chr4 | 6083158 | 6083208 |
| chr4 | 6086114 | 6086164 |
| chr4 | 6429501 | 6429551 |
| chr4 | 7190666 | 7190716 |
| chr4 | 8210971 | 8211021 |
| chr4 | 8743364 | 8743414 |
| chr4 | 9433459 | 9433509 |
| chr4 | 10302606 | 10302656 |
| chr4 | 17891457 | 17891507 |
| chr4 | 19676635 | 19676685 |
| chr4 | 24064180 | 24064230 |
| chr4 | 25284538 | 25284588 |
| chr4 | 25867838 | 25867888 |
| chr4 | 31760439 | 31760489 |
| chr4 | 33743107 | 33743157 |
| chr4 | 36316214 | 36316264 |
| chr4 | 37430378 | 37430428 |
| chr4 | 38617257 | 38617307 |
| chr4 | 38687965 | 38688015 |
| chr4 | 39172103 | 39172153 |
| chr4 | 40075662 | 40075712 |
| chr4 | 41986183 | 41986233 |
| chr4 | 43458321 | 43458371 |
| chr4 | 43882990 | 43883040 |
| chr4 | 44591443 | 44591493 |
| chr4 | 46426251 | 46426301 |
| chr4 | 46782153 | 46782203 |
| chr4 | 57416323 | 57416373 |
| chr11 | 65229711 | 65229761 |
| chr4 | 59523525 | 59523575 |
| chr11 | 65268598 | 65268648 |
| chr11 | 65271986 | 65272036 |
| chr4 | 65949075 | 65949125 |
| chr4 | 69226257 | 69226307 |
| chr4 | 72162987 | 72163037 |
| chr4 | 73458954 | 73459004 |
| chr4 | 77296608 | 77296658 |
| chr4 | 77304481 | 77304531 |
| chr4 | 77352013 | 77352063 |
| chr4 | 78554724 | 78554774 |
| chr4 | 79926832 | 79926882 |
| chr4 | 81355750 | 81355800 |
| chr11 | 65577159 | 65577209 |
| chr4 | 82793544 | 82793594 |
| chr4 | 83776071 | 83776121 |
| chr4 | 84111918 | 84111968 |
| chr4 | 86769251 | 86769301 |
| chr4 | 87992869 | 87992919 |
| chr4 | 98289148 | 98289198 |
| chr4 | 98591577 | 98591627 |
| chr4 | 99710973 | 99711023 |
| chr4 | 100320943 | 100320993 |
| chr4 | 100329483 | 100329533 |
| chr4 | 102882169 | 102882219 |
| chr4 | 104217720 | 104217770 |
| chr4 | 110762298 | 110762348 |
| chr4 | 111059573 | 111059623 |
| chr4 | 117969564 | 117969614 |
| chr4 | 124513038 | 124513088 |
| chr4 | 124731975 | 124732025 |
| chr11 | 66435761 | 66435811 |
| chr4 | 130080378 | 130080428 |
| chr4 | 133184206 | 133184256 |
| chr4 | 133189495 | 133189545 |
| chr4 | 133498126 | 133498176 |
| chr4 | 134756686 | 134756736 |
| chr4 | 135529961 | 135530011 |
| chr4 | 136007219 | 136007269 |
| chr4 | 140000884 | 140000934 |
| chr4 | 140661433 | 140661483 |
| chr4 | 140668253 | 140668303 |
| chr4 | 142515534 | 142515584 |
| chr4 | 142515263 | 142515313 |
| chr4 | 144158335 | 144158385 |
| chr4 | 147748273 | 147748323 |
| chr4 | 152052447 | 152052497 |
| chr4 | 154619764 | 154619814 |
| chr4 | 158890212 | 158890262 |
| chr4 | 160186307 | 160186357 |
| chr4 | 160919662 | 160919712 |
| chr4 | 162228125 | 162228175 |
| chr4 | 163496714 | 163496764 |
| chr11 | 67205161 | 67205211 |
| chr4 | 164863422 | 164863472 |
| chr4 | 166532817 | 166532867 |
| chr4 | 167213535 | 167213585 |
| chr4 | 169647002 | 169647052 |
| chr4 | 171947817 | 171947867 |
| chr4 | 171961991 | 171962041 |
| chr4 | 174159601 | 174159651 |
| chr4 | 174223258 | 174223308 |
| chr4 | 174905436 | 174905486 |
| chr4 | 176200658 | 176200708 |
| chr4 | 176546670 | 176546720 |
| chr4 | 176815046 | 176815096 |
| chr4 | 179124771 | 179124821 |
| chr4 | 179654979 | 179655029 |
| chr4 | 179892171 | 179892221 |
| chr4 | 179979455 | 179979505 |
| chr4 | 181282297 | 181282347 |
| chr4 | 182182875 | 182182925 |
| chr4 | 184372933 | 184372983 |
| chr4 | 185023448 | 185023498 |
| chr4 | 185170015 | 185170065 |
| chr4 | 185171342 | 185171392 |
| chr4 | 185171569 | 185171619 |
| chr4 | 185333536 | 185333586 |
| chr4 | 185364069 | 185364119 |
| chr4 | 185364243 | 185364293 |
| chr4 | 185364500 | 185364550 |
| chr4 | 186894580 | 186894630 |
| chr11 | 68303328 | 68303378 |
| chr4 | 188362110 | 188362160 |
| chr4 | 188362485 | 188362535 |
| chr4 | 188362567 | 188362617 |
| chr4 | 188888579 | 188888629 |
| chr4 | 188984139 | 188984189 |
| chr4 | 189593944 | 189593994 |
| chr4 | 189808416 | 189808466 |
| chr4 | 190247440 | 190247490 |
| chr4 | 190248113 | 190248163 |
| chr4 | 190556677 | 190556727 |
| chr4 | 191221781 | 191221831 |
| chr5 | 256482 | 256532 |
| chr11 | 68517783 | 68517833 |
| chr5 | 698725 | 698775 |
| chr5 | 1243146 | 1243196 |
| chr5 | 1243461 | 1243511 |
| chr5 | 1428571 | 1428621 |
| chr5 | 2320972 | 2321022 |
| chr5 | 2366984 | 2367034 |
| chr5 | 3710941 | 3710991 |
| chr5 | 3875646 | 3875696 |
| chr5 | 5419486 | 5419536 |
| chr5 | 5621277 | 5621327 |
| chr5 | 5752189 | 5752239 |
| chr5 | 6562729 | 6562779 |
| chr5 | 6564930 | 6564980 |
| chr5 | 12025015 | 12025065 |
| chr5 | 13942937 | 13942987 |
| chr5 | 14558084 | 14558134 |
| chr5 | 16831453 | 16831503 |
| chr5 | 20662007 | 20662057 |
| chr5 | 21061501 | 21061551 |
| chr5 | 21315852 | 21315902 |
| chr11 | 69591881 | 69591931 |
| chr11 | 69591678 | 69591728 |
| chr11 | 69667991 | 69668041 |
| chr5 | 25309264 | 25309314 |
| chr5 | 25430145 | 25430195 |
| chr5 | 25898770 | 25898820 |
| chr5 | 25904291 | 25904341 |
| chr5 | 26521626 | 26521676 |
| chr5 | 26826732 | 26826782 |
| chr5 | 27958909 | 27958959 |
| chr5 | 28748974 | 28749024 |
| chr5 | 29341077 | 29341127 |
| chr5 | 29342489 | 29342539 |
| chr5 | 33292158 | 33292208 |
| chr5 | 34566635 | 34566685 |
| chr5 | 35767875 | 35767925 |
| chr5 | 36491949 | 36491999 |
| chr5 | 37173050 | 37173100 |
| chr5 | 37301032 | 37301082 |
| chr5 | 37303886 | 37303936 |
| chr5 | 37308658 | 37308708 |
| chr5 | 40907236 | 40907286 |
| chr5 | 43053871 | 43053921 |
| chr5 | 43110470 | 43110520 |
| chr5 | 52894591 | 52894641 |
| chr5 | 53204858 | 53204908 |
| chr5 | 55485193 | 55485243 |
| chr5 | 55743123 | 55743173 |
| chr5 | 57246987 | 57247037 |
| chr5 | 58207802 | 58207852 |
| chr5 | 59093596 | 59093646 |
| chr5 | 62636397 | 62636447 |
| chr5 | 66182604 | 66182654 |
| chr5 | 66182420 | 66182470 |
| chr5 | 67129551 | 67129601 |
| chr5 | 68557784 | 68557834 |
| chr5 | 73844173 | 73844223 |
| chr5 | 76190645 | 76190695 |
| chr5 | 76741345 | 76741395 |
| chr5 | 78969219 | 78969269 |
| chr5 | 80159780 | 80159830 |
| chr5 | 80967299 | 80967349 |
| chr5 | 85612020 | 85612070 |
| chr5 | 86620199 | 86620249 |
| chr5 | 86620495 | 86620545 |
| chr11 | 71212075 | 71212125 |
| chr11 | 71213262 | 71213312 |
| chr5 | 93944972 | 93945022 |
| chr5 | 98251474 | 98251524 |
| chr5 | 99911734 | 99911784 |
| chr5 | 104080994 | 104081044 |
| chr5 | 105996596 | 105996646 |
| chr11 | 71600566 | 71600616 |
| chr5 | 118442364 | 118442414 |
| chr5 | 122039996 | 122040046 |
| chr5 | 124372161 | 124372211 |
| chr5 | 124384988 | 124385038 |
| chr11 | 72237070 | 72237120 |
| chr5 | 129714940 | 129714990 |
| chr5 | 131593827 | 131593877 |
| chr5 | 133889878 | 133889928 |
| chr5 | 138029222 | 138029272 |
| chr5 | 138643314 | 138643364 |
| chr5 | 139652667 | 139652717 |
| chr5 | 140695468 | 140695518 |
| chr5 | 145245185 | 145245235 |
| chr11 | 73582975 | 73583025 |
| chr11 | 73971842 | 73971892 |
| chr5 | 151255899 | 151255949 |
| chr5 | 157928299 | 157928349 |
| chr5 | 157978767 | 157978817 |
| chr5 | 164376827 | 164376877 |
| chr5 | 165010850 | 165010900 |
| chr5 | 168927991 | 168928041 |
| chr5 | 171065026 | 171065076 |
| chr5 | 171463468 | 171463518 |
| chr5 | 172017491 | 172017541 |
| chr5 | 173498268 | 173498318 |
| chr5 | 174009991 | 174010041 |
| chr5 | 174469992 | 174470042 |
| chr5 | 174613900 | 174613950 |
| chr5 | 175137457 | 175137507 |
| chr10 | 14861303 | 14861353 |
| chr5 | 178379857 | 178379907 |
| chr5 | 178996603 | 178996653 |
| chr5 | 179348060 | 179348110 |
| chr5 | 180367875 | 180367925 |
| chr5 | 180368012 | 180368062 |
| chr5 | 180587841 | 180587891 |
| chr5 | 180600261 | 180600311 |
| chr6 | 728494 | 728544 |
| chr6 | 913365 | 913415 |
| chr11 | 77084915 | 77084965 |
| chr6 | 9609436 | 9609486 |
| chr6 | 10421183 | 10421233 |
| chr6 | 10913493 | 10913543 |
| chr6 | 14163046 | 14163096 |
| chr6 | 14998714 | 14998764 |
| chr6 | 15320745 | 15320795 |
| chr6 | 16503239 | 16503289 |
| chr6 | 17337986 | 17338036 |
| chr6 | 18198419 | 18198469 |
| chr6 | 20553687 | 20553737 |
| chr6 | 23408690 | 23408740 |
| chr6 | 25456615 | 25456665 |
| chr6 | 26096118 | 26096168 |
| chr6 | 26425471 | 26425521 |
| chr6 | 27202998 | 27203048 |
| chr6 | 27284369 | 27284419 |
| chr6 | 27739211 | 27739261 |
| chr6 | 27975954 | 27976004 |
| chr6 | 28942460 | 28942510 |
| chr6 | 28942969 | 28943019 |
| chr6 | 29759374 | 29759424 |
| chr6 | 30105403 | 30105453 |
| chr6 | 30530038 | 30530088 |
| chr6 | 34336789 | 34336839 |
| chr10 | 888625 | 888675 |
| chr6 | 35115605 | 35115655 |
| chr6 | 37008654 | 37008704 |
| chr6 | 37946584 | 37946634 |
| chr6 | 38174002 | 38174052 |
| chr6 | 42178912 | 42178962 |
| chr6 | 42407656 | 42407706 |
| chr6 | 44435516 | 44435566 |
| chr6 | 52699458 | 52699508 |
| chr6 | 52997389 | 52997439 |
| chr6 | 53000995 | 53001045 |
| chr11 | 87838099 | 87838149 |
| chr6 | 56963629 | 56963679 |
| chr6 | 57681279 | 57681329 |
| chr6 | 62689045 | 62689095 |
| chr6 | 63822591 | 63822641 |
| chr6 | 64171815 | 64171865 |
| chr6 | 64611756 | 64611806 |
| chr6 | 66920360 | 66920410 |
| chr6 | 66948363 | 66948413 |
| chr6 | 69744895 | 69744945 |
| chr6 | 70245610 | 70245660 |
| chr6 | 70439639 | 70439689 |
| chr6 | 71477016 | 71477066 |
| chr6 | 73869114 | 73869164 |
| chr6 | 80465814 | 80465864 |
| chr6 | 80475146 | 80475196 |
| chr6 | 81764187 | 81764237 |
| chr11 | 91615420 | 91615470 |
| chr6 | 84785142 | 84785192 |
| chr6 | 84790714 | 84790764 |
| chr6 | 96013592 | 96013642 |
| chr6 | 101525035 | 101525085 |
| chr6 | 101824305 | 101824355 |
| chr6 | 103403562 | 103403612 |
| chr6 | 106398279 | 106398329 |
| chr6 | 108332251 | 108332301 |
| chr6 | 110246491 | 110246541 |
| chr6 | 111398568 | 111398618 |
| chr11 | 94557072 | 94557122 |
| chr6 | 112729491 | 112729541 |
| chr11 | 94857374 | 94857424 |
| chr6 | 116186093 | 116186143 |
| chr6 | 123110279 | 123110329 |
| chr6 | 126628282 | 126628332 |
| chr6 | 127894901 | 127894951 |
| chr6 | 128522920 | 128522970 |
| chr6 | 128804568 | 128804618 |
| chr6 | 131603056 | 131603106 |
| chr11 | 96788761 | 96788811 |
| chr11 | 97263542 | 97263592 |
| chr11 | 97382143 | 97382193 |
| chr11 | 97474790 | 97474840 |
| chr11 | 97477923 | 97477973 |
| chr11 | 97480148 | 97480198 |
| chr6 | 136892550 | 136892600 |
| chr6 | 138310191 | 138310241 |
| chr6 | 139796646 | 139796696 |
| chr11 | 98172579 | 98172629 |
| chr6 | 142031461 | 142031511 |
| chr6 | 150520153 | 150520203 |
| chr6 | 152295686 | 152295736 |
| chr6 | 154139444 | 154139494 |
| chr6 | 157699280 | 157699330 |
| chr6 | 158848861 | 158848911 |
| chr6 | 158880657 | 158880707 |
| chr6 | 159382256 | 159382306 |
| chr6 | 160430103 | 160430153 |
| chr6 | 163224049 | 163224099 |
| chr6 | 163384604 | 163384654 |
| chr6 | 163384804 | 163384854 |
| chr6 | 167006595 | 167006645 |
| chr6 | 167038839 | 167038889 |
| chr6 | 167039107 | 167039157 |
| chr6 | 167039337 | 167039387 |
| chr6 | 167303587 | 167303637 |
| chr6 | 168314321 | 168314371 |
| chr6 | 168328009 | 168328059 |
| chr6 | 168328246 | 168328296 |
| chr6 | 168410745 | 168410795 |
| chr6 | 168860792 | 168860842 |
| chr11 | 105728036 | 105728086 |
| chr11 | 105740918 | 105740968 |
| chr6 | 169039921 | 169039971 |
| chr6 | 169040786 | 169040836 |
| chr6 | 169041097 | 169041147 |
| chr6 | 169041324 | 169041374 |
| chr10 | 16405961 | 16406011 |
| chr6 | 169934354 | 169934404 |
| chr10 | 16433826 | 16433876 |
| chr6 | 170597993 | 170598043 |
| chr7 | 145377 | 145427 |
| chr7 | 1184279 | 1184329 |
| chr11 | 107379019 | 107379069 |
| chr7 | 1565481 | 1565531 |
| chr7 | 2086044 | 2086094 |
| chr7 | 3264213 | 3264263 |
| chr7 | 4198710 | 4198760 |
| chr7 | 4279874 | 4279924 |
| chr7 | 4748760 | 4748810 |
| chr7 | 4826829 | 4826879 |
| chr7 | 5049792 | 5049842 |
| chr7 | 5056113 | 5056163 |
| chr7 | 6255463 | 6255513 |
| chr7 | 6510762 | 6510812 |
| chr7 | 11776211 | 11776261 |
| chr7 | 12313752 | 12313802 |
| chr7 | 14856258 | 14856308 |
| chr7 | 15820233 | 15820283 |
| chr7 | 19124216 | 19124266 |
| chr7 | 20088989 | 20089039 |
| chr7 | 26759479 | 26759529 |
| chr7 | 29199923 | 29199973 |
| chr7 | 32896464 | 32896514 |
| chr7 | 32922137 | 32922187 |
| chr11 | 111790318 | 111790368 |
| chr7 | 38890776 | 38890826 |
| chr7 | 39940527 | 39940577 |
| chr7 | 40497790 | 40497840 |
| chr7 | 42487148 | 42487198 |
| chr7 | 42712888 | 42712938 |
| chr7 | 42857075 | 42857125 |
| chr7 | 42964743 | 42964793 |
| chr7 | 44767423 | 44767473 |
| chr7 | 44768334 | 44768384 |
| chr7 | 46864426 | 46864476 |
| chr7 | 47277063 | 47277113 |
| chr7 | 47276843 | 47276893 |
| chr7 | 50868184 | 50868234 |
| chr7 | 51228350 | 51228400 |
| chr7 | 51835806 | 51835856 |
| chr7 | 55418979 | 55419029 |
| chr7 | 65936082 | 65936132 |
| chr7 | 66233982 | 66234032 |
| chr7 | 66811954 | 66812004 |
| chr7 | 66893714 | 66893764 |
| chr7 | 67220930 | 67220980 |
| chr7 | 67474895 | 67474945 |
| chr7 | 71208500 | 71208550 |
| chr7 | 72716643 | 72716693 |
| chr7 | 72954538 | 72954588 |
| chr7 | 75195014 | 75195064 |
| chr7 | 75637427 | 75637477 |
| chr7 | 76984134 | 76984184 |
| chr7 | 77317121 | 77317171 |
| chr7 | 78535856 | 78535906 |
| chr7 | 80222553 | 80222603 |
| chr7 | 87469330 | 87469380 |
| chr7 | 90204974 | 90205024 |
| chr7 | 91668428 | 91668478 |
| chr7 | 97719940 | 97719990 |
| chr7 | 98027856 | 98027906 |
| chr7 | 99036964 | 99037014 |
| chr7 | 99216896 | 99216946 |
| chr7 | 100537350 | 100537400 |
| chr7 | 100616621 | 100616671 |
| chr7 | 101309865 | 101309915 |
| chr7 | 102124333 | 102124383 |
| chr7 | 103374725 | 103374775 |
| chr7 | 103382448 | 103382498 |
| chr7 | 105745327 | 105745377 |
| chr7 | 108105961 | 108106011 |
| chr7 | 108105660 | 108105710 |
| chr7 | 118884070 | 118884120 |
| chr7 | 120032332 | 120032382 |
| chr7 | 121120544 | 121120594 |
| chr7 | 122108392 | 122108442 |
| chr7 | 124669591 | 124669641 |
| chr7 | 124837681 | 124837731 |
| chr7 | 126708221 | 126708271 |
| chr7 | 127787213 | 127787263 |
| chr7 | 129916822 | 129916872 |
| chr7 | 132326854 | 132326904 |
| chr7 | 133976471 | 133976521 |
| chr7 | 136725685 | 136725735 |
| chr7 | 139506939 | 139506989 |
| chr7 | 143748226 | 143748276 |
| chr7 | 147303681 | 147303731 |
| chr7 | 147615956 | 147616006 |
| chr7 | 147746302 | 147746352 |
| chr7 | 148412379 | 148412429 |
| chr7 | 148415243 | 148415293 |
| chr7 | 149474201 | 149474251 |
| chr7 | 149811195 | 149811245 |
| chr7 | 150325271 | 150325321 |
| chr7 | 150355749 | 150355799 |
| chr7 | 150373286 | 150373336 |
| chr7 | 150561164 | 150561214 |
| chr11 | 119503110 | 119503160 |
| chr7 | 154088238 | 154088288 |
| chr7 | 156535150 | 156535200 |
| chr7 | 156739834 | 156739884 |
| chr7 | 157682380 | 157682430 |
| chr8 | 395160 | 395210 |
| chr8 | 837621 | 837671 |
| chr8 | 852451 | 852501 |
| chr8 | 882970 | 883020 |
| chr8 | 1300077 | 1300127 |
| chr8 | 1842158 | 1842208 |
| chr8 | 2688992 | 2689042 |
| chr8 | 2728861 | 2728911 |
| chr8 | 4436771 | 4436821 |
| chr8 | 4876287 | 4876337 |
| chr8 | 5133978 | 5134028 |
| chr8 | 5624542 | 5624592 |
| chr8 | 6663239 | 6663289 |
| chr8 | 9301392 | 9301442 |
| chr8 | 9425976 | 9426026 |
| chr8 | 9779830 | 9779880 |
| chr8 | 11397772 | 11397822 |
| chr8 | 11448707 | 11448757 |
| chr8 | 11692038 | 11692088 |
| chr8 | 12770563 | 12770613 |
| chr8 | 14309135 | 14309185 |
| chr8 | 15450176 | 15450226 |
| chr8 | 19037273 | 19037323 |
| chr8 | 19853889 | 19853939 |
| chr8 | 21771256 | 21771306 |
| chr8 | 22697120 | 22697170 |
| chr8 | 22979711 | 22979761 |
| chr8 | 23047055 | 23047105 |
| chr8 | 24606508 | 24606558 |
| chr8 | 25510992 | 25511042 |
| chr8 | 26083838 | 26083888 |
| chr8 | 26083748 | 26083798 |
| chr8 | 27045286 | 27045336 |
| chr8 | 27770505 | 27770555 |
| chr8 | 28284000 | 28284050 |
| chr8 | 28498904 | 28498954 |
| chr8 | 30050171 | 30050221 |
| chr8 | 30992710 | 30992760 |
| chr8 | 34494590 | 34494640 |
| chr8 | 34877997 | 34878047 |
| chr8 | 36329118 | 36329168 |
| chr8 | 38078450 | 38078500 |
| chr8 | 39535845 | 39535895 |
| chr8 | 40081174 | 40081224 |
| chr8 | 42050981 | 42051031 |
| chr8 | 42589204 | 42589254 |
| chr11 | 124779706 | 124779756 |
| chr8 | 43077062 | 43077112 |
| chr8 | 48144236 | 48144286 |
| chr11 | 124866300 | 124866350 |
| chr8 | 52944963 | 52945013 |
| chr8 | 54024766 | 54024816 |
| chr8 | 54376459 | 54376509 |
| chr8 | 56035916 | 56035966 |
| chr8 | 56038231 | 56038281 |
| chr8 | 59625641 | 59625691 |
| chr8 | 64061827 | 64061877 |
| chr8 | 65046081 | 65046131 |
| chr8 | 66844433 | 66844483 |
| chr8 | 68591979 | 68592029 |
| chr8 | 69805035 | 69805085 |
| chr8 | 69892379 | 69892429 |
| chr8 | 75309197 | 75309247 |
| chr8 | 85113704 | 85113754 |
| chr8 | 87038216 | 87038266 |
| chr8 | 89786569 | 89786619 |
| chr8 | 93384807 | 93384857 |
| chr8 | 98515789 | 98515839 |
| chr8 | 98581455 | 98581505 |
| chr8 | 101184624 | 101184674 |
| chr8 | 103507373 | 103507423 |
| chr8 | 105356661 | 105356711 |
| chr8 | 105356415 | 105356465 |
| chr8 | 112791436 | 112791486 |
| chr8 | 113820262 | 113820312 |
| chr11 | 128752469 | 128752519 |
| chr11 | 128819874 | 128819924 |
| chr8 | 118119215 | 118119265 |
| chr8 | 120656542 | 120656592 |
| chr8 | 120939691 | 120939741 |
| chr8 | 122902045 | 122902095 |
| chr8 | 123409029 | 123409079 |
| chr10 | 19173537 | 19173587 |
| chr8 | 124015769 | 124015819 |
| chr11 | 129469593 | 129469643 |
| chr8 | 131001847 | 131001897 |
| chr8 | 132193387 | 132193437 |
| chr8 | 132545627 | 132545677 |
| chr8 | 133186232 | 133186282 |
| chr8 | 134278512 | 134278562 |
| chr8 | 137401134 | 137401184 |
| chr8 | 137437248 | 137437298 |
| chr8 | 139246315 | 139246365 |
| chr8 | 140215165 | 140215215 |
| chr8 | 140438957 | 140439007 |
| chr8 | 140909259 | 140909309 |
| chr11 | 129855764 | 129855814 |
| chr8 | 142017431 | 142017481 |
| chr8 | 142130704 | 142130754 |
| chr8 | 142214170 | 142214220 |
| chr8 | 142385247 | 142385297 |
| chr8 | 142442457 | 142442507 |
| chr11 | 130353664 | 130353714 |
| chr8 | 144327759 | 144327809 |
| chr9 | 3567979 | 3568029 |
| chr9 | 3891195 | 3891245 |
| chr9 | 3891098 | 3891148 |
| chr9 | 3915610 | 3915660 |
| chr9 | 5711451 | 5711501 |
| chr9 | 6713465 | 6713515 |
| chr9 | 6718490 | 6718540 |
| chr9 | 9701044 | 9701094 |
| chr9 | 11090325 | 11090375 |
| chr9 | 11295401 | 11295451 |
| chr9 | 12157161 | 12157211 |
| chr9 | 12449787 | 12449837 |
| chr9 | 19108028 | 19108078 |
| chr9 | 26136016 | 26136066 |
| chr9 | 26351897 | 26351947 |
| chr9 | 26595158 | 26595208 |
| chr9 | 27626074 | 27626124 |
| chr9 | 27993147 | 27993197 |
| chr9 | 29341931 | 29341981 |
| chr9 | 30335756 | 30335806 |
| chr9 | 31839437 | 31839487 |
| chr9 | 32174842 | 32174892 |
| chr9 | 32909794 | 32909844 |
| chr9 | 34118817 | 34118867 |
| chr9 | 34147287 | 34147337 |
| chr11 | 133253322 | 133253372 |
| chr11 | 133252830 | 133252880 |
| chr9 | 36527451 | 36527501 |
| chr9 | 37050530 | 37050580 |
| chr9 | 39311485 | 39311535 |
| chr9 | 41246676 | 41246726 |
| chr9 | 42163936 | 42163986 |
| chr9 | 63599087 | 63599137 |
| chr9 | 67686928 | 67686978 |
| chr9 | 67879966 | 67880016 |
| chr9 | 67882339 | 67882389 |
| chr9 | 68289256 | 68289306 |
| chr9 | 69133093 | 69133143 |
| chr9 | 72114832 | 72114882 |
| chr9 | 72280891 | 72280941 |
| chr9 | 73591487 | 73591537 |
| chr9 | 74090361 | 74090411 |
| chr9 | 74440279 | 74440329 |
| chr9 | 75673047 | 75673097 |
| chr11 | 133642690 | 133642740 |
| chr9 | 83906444 | 83906494 |
| chr9 | 84009162 | 84009212 |
| chr9 | 84009271 | 84009321 |
| chr9 | 84009646 | 84009696 |
| chr9 | 86440297 | 86440347 |
| chr9 | 86644643 | 86644693 |
| chr9 | 86644557 | 86644607 |
| chr9 | 87037441 | 87037491 |
| chr11 | 133858765 | 133858815 |
| chr9 | 88567294 | 88567344 |
| chr9 | 89665152 | 89665202 |
| chr9 | 91086460 | 91086510 |
| chr9 | 92026656 | 92026706 |
| chr9 | 92389776 | 92389826 |
| chr9 | 92773216 | 92773266 |
| chr9 | 92804767 | 92804817 |
| chr9 | 92804936 | 92804986 |
| chr9 | 93525460 | 93525510 |
| chr9 | 93823868 | 93823918 |
| chr9 | 93847190 | 93847240 |
| chr9 | 94519257 | 94519307 |
| chr9 | 94853978 | 94854028 |
| chr9 | 95216987 | 95217037 |
| chr10 | 21129479 | 21129529 |
| chr9 | 95871806 | 95871856 |
| chr12 | 206235 | 206285 |
| chr9 | 96261816 | 96261866 |
| chr9 | 96262259 | 96262309 |
| chr9 | 96262410 | 96262460 |
| chr9 | 96516831 | 96516881 |
| chr9 | 96896361 | 96896411 |
| chr12 | 318271 | 318321 |
| chr9 | 99635436 | 99635486 |
| chr9 | 99635515 | 99635565 |
| chr9 | 101265608 | 101265658 |
| chr9 | 101271803 | 101271853 |
| chr9 | 101468331 | 101468381 |
| chr9 | 109597624 | 109597674 |
| chr12 | 698565 | 698615 |
| chr9 | 109833274 | 109833324 |
| chr9 | 112700396 | 112700446 |
| chr9 | 112940965 | 112941015 |
| chr9 | 113598091 | 113598141 |
| chr9 | 118657528 | 118657578 |
| chr9 | 119401902 | 119401952 |
| chr9 | 120674529 | 120674579 |
| chr9 | 122554918 | 122554968 |
| chr9 | 124809363 | 124809413 |
| chr9 | 125990956 | 125991006 |
| chr9 | 127645306 | 127645356 |
| chr9 | 128890372 | 128890422 |
| chr12 | 2265067 | 2265117 |
| chr9 | 130013737 | 130013787 |
| chr9 | 131373806 | 131373856 |
| chr9 | 131383844 | 131383894 |
| chr9 | 131871970 | 131872020 |
| chr9 | 131908284 | 131908334 |
| chr9 | 132637684 | 132637734 |
| chr9 | 132938734 | 132938784 |
| chr9 | 133866710 | 133866760 |
| chr9 | 134563427 | 134563477 |
| chr9 | 134887453 | 134887503 |
| chr9 | 135126728 | 135126778 |
| chr9 | 135389171 | 135389221 |
| chr9 | 135789816 | 135789866 |
| chr10 | 21769230 | 21769280 |
| chr12 | 6080881 | 6080931 |
| chr12 | 6822395 | 6822445 |
| chr12 | 7148199 | 7148249 |
| chr12 | 8296307 | 8296357 |
| chr12 | 8317135 | 8317185 |
| chr12 | 9651616 | 9651666 |
| chr12 | 10081318 | 10081368 |
| chr12 | 10175816 | 10175866 |
| chr12 | 10176215 | 10176265 |
| chr12 | 10176354 | 10176404 |
| chr12 | 10273838 | 10273888 |
| chr12 | 11636891 | 11636941 |
| chr10 | 23124399 | 23124449 |
| chr10 | 23673113 | 23673163 |
| chr12 | 22419838 | 22419888 |
| chr12 | 24442689 | 24442739 |
| chr12 | 25861147 | 25861197 |
| chr12 | 29833202 | 29833252 |
| chr12 | 30861731 | 30861781 |
| chr12 | 30861449 | 30861499 |
| chr12 | 31774148 | 31774198 |
| chr12 | 31774976 | 31775026 |
| chr12 | 32468012 | 32468062 |
| chr12 | 33728005 | 33728055 |
| chr12 | 40613466 | 40613516 |
| chr12 | 43865354 | 43865404 |
| chr12 | 44567983 | 44568033 |
| chr10 | 26068046 | 26068096 |
| chr12 | 47815136 | 47815186 |
| chr12 | 47824452 | 47824502 |
| chr12 | 48771175 | 48771225 |
| chr12 | 48965188 | 48965238 |
| chr12 | 49631606 | 49631656 |
| chr12 | 51217048 | 51217098 |
| chr12 | 56376938 | 56376988 |
| chr12 | 59068082 | 59068132 |
| chr12 | 60730709 | 60730759 |
| chr12 | 67658951 | 67659001 |
| chr12 | 68865794 | 68865844 |
| chr12 | 69149355 | 69149405 |
| chr12 | 73974344 | 73974394 |
| chr12 | 76078861 | 76078911 |
| chr10 | 29285682 | 29285732 |
| chr10 | 29300672 | 29300722 |
| chr12 | 86284044 | 86284094 |
| chr10 | 1211921 | 1211971 |
| chr12 | 93435796 | 93435846 |
| chr12 | 94524482 | 94524532 |
| chr12 | 103177935 | 103177985 |
| chr12 | 103917345 | 103917395 |
| chr12 | 106105920 | 106105970 |
| chr12 | 106685400 | 106685450 |
| chr12 | 106812813 | 106812863 |
| chr12 | 108591345 | 108591395 |
| chr12 | 112462044 | 112462094 |
| chr12 | 113426875 | 113426925 |
| chr12 | 114760939 | 114760989 |
| chr12 | 115739174 | 115739224 |
| chr12 | 118282932 | 118282982 |
| chr12 | 118477844 | 118477894 |
| chr12 | 118557227 | 118557277 |
| chr12 | 119820254 | 119820304 |
| chr12 | 120697937 | 120697987 |
| chr12 | 122383187 | 122383237 |
| chr12 | 122404178 | 122404228 |
| chr12 | 122730405 | 122730455 |
| chr12 | 122730225 | 122730275 |
| chr12 | 123246905 | 123246955 |
| chr12 | 123629231 | 123629281 |
| chr12 | 123773287 | 123773337 |
| chr12 | 123813227 | 123813277 |
| chr12 | 124819822 | 124819872 |
| chr12 | 126559727 | 126559777 |
| chr12 | 126736419 | 126736469 |
| chr12 | 126782831 | 126782881 |
| chr12 | 127043789 | 127043839 |
| chr12 | 127285303 | 127285353 |
| chr12 | 127367738 | 127367788 |
| chr12 | 127770661 | 127770711 |
| chr12 | 128390825 | 128390875 |
| chr12 | 128660138 | 128660188 |
| chr12 | 128796120 | 128796170 |
| chr12 | 129087427 | 129087477 |
| chr12 | 129087084 | 129087134 |
| chr12 | 129292767 | 129292817 |
| chr10 | 34967818 | 34967868 |
| chr12 | 130054855 | 130054905 |
| chr12 | 130131599 | 130131649 |
| chr12 | 130645668 | 130645718 |
| chr12 | 130671637 | 130671687 |
| chr12 | 130791990 | 130792040 |
| chr12 | 130798380 | 130798430 |
| chr12 | 130975650 | 130975700 |
| chr12 | 131393291 | 131393341 |
| chr12 | 131653164 | 131653214 |
| chr12 | 131653341 | 131653391 |
| chr12 | 131863091 | 131863141 |
| chr10 | 35498550 | 35498600 |
| chr13 | 18176464 | 18176514 |
| chr13 | 18187302 | 18187352 |
| chr13 | 20110580 | 20110630 |
| chr13 | 20110509 | 20110559 |
| chr13 | 21664136 | 21664186 |
| chr13 | 22078920 | 22078970 |
| chr13 | 22861829 | 22861879 |
| chr13 | 23012346 | 23012396 |
| chr13 | 23055379 | 23055429 |
| chr13 | 23659742 | 23659792 |
| chr10 | 36624838 | 36624888 |
| chr13 | 25460577 | 25460627 |
| chr13 | 29712913 | 29712963 |
| chr13 | 29949747 | 29949797 |
| chr13 | 33175567 | 33175617 |
| chr13 | 34830722 | 34830772 |
| chr13 | 35188419 | 35188469 |
| chr13 | 41453453 | 41453503 |
| chr13 | 43204755 | 43204805 |
| chr10 | 38219594 | 38219644 |
| chr13 | 43487308 | 43487358 |
| chr10 | 38381296 | 38381346 |
| chr13 | 47676700 | 47676750 |
| chr13 | 47677638 | 47677688 |
| chr13 | 47945447 | 47945497 |
| chr13 | 48199533 | 48199583 |
| chr10 | 38933647 | 38933697 |
| chr13 | 49347648 | 49347698 |
| chr13 | 50241942 | 50241992 |
| chr13 | 53956347 | 53956397 |
| chr13 | 55343182 | 55343232 |
| chr13 | 58956760 | 58956810 |
| chr13 | 60043392 | 60043442 |
| chr13 | 62246390 | 62246440 |
| chr13 | 62898016 | 62898066 |
| chr13 | 63006474 | 63006524 |
| chr13 | 64990503 | 64990553 |
| chr13 | 64990328 | 64990378 |
| chr13 | 65743396 | 65743446 |
| chr13 | 66629427 | 66629477 |
| chr13 | 67080688 | 67080738 |
| chr13 | 67144550 | 67144600 |
| chr13 | 67709234 | 67709284 |
| chr13 | 70495916 | 70495966 |
| chr13 | 74754723 | 74754773 |
| chr13 | 77748744 | 77748794 |
| chr13 | 80411688 | 80411738 |
| chr13 | 80882686 | 80882736 |
| chr13 | 80970219 | 80970269 |
| chr13 | 82187806 | 82187856 |
| chr13 | 82368725 | 82368775 |
| chr13 | 83413791 | 83413841 |
| chr13 | 85093609 | 85093659 |
| chr13 | 86267888 | 86267938 |
| chr13 | 89276974 | 89277024 |
| chr13 | 89276767 | 89276817 |
| chr13 | 90380964 | 90381014 |
| chr13 | 93053669 | 93053719 |
| chr13 | 98822595 | 98822645 |
| chr13 | 99044495 | 99044545 |
| chr13 | 99114079 | 99114129 |
| chr13 | 100021462 | 100021512 |
| chr13 | 101043270 | 101043320 |
| chr13 | 103423595 | 103423645 |
| chr13 | 107236910 | 107236960 |
| chr13 | 108913909 | 108913959 |
| chr13 | 109946413 | 109946463 |
| chr13 | 110464198 | 110464248 |
| chr13 | 110811501 | 110811551 |
| chr13 | 111386027 | 111386077 |
| chr13 | 111410141 | 111410191 |
| chr13 | 111894512 | 111894562 |
| chr13 | 112766290 | 112766340 |
| chr14 | 19132231 | 19132281 |
| chr14 | 19279058 | 19279108 |
| chr14 | 22748871 | 22748921 |
| chr14 | 24792501 | 24792551 |
| chr14 | 32952870 | 32952920 |
| chr14 | 33008776 | 33008826 |
| chr14 | 42845289 | 42845339 |
| chr14 | 42845428 | 42845478 |
| chr14 | 43114922 | 43114972 |
| chr14 | 43582701 | 43582751 |
| chr14 | 46394305 | 46394355 |
| chr14 | 48255303 | 48255353 |
| chr10 | 49660196 | 49660246 |
| chr14 | 49417061 | 49417111 |
| chr14 | 49623174 | 49623224 |
| chr14 | 53514748 | 53514798 |
| chr14 | 53515273 | 53515323 |
| chr10 | 50093323 | 50093373 |
| chr14 | 55449233 | 55449283 |
| chr14 | 55449822 | 55449872 |
| chr14 | 61365643 | 61365693 |
| chr14 | 61435048 | 61435098 |
| chr14 | 61570986 | 61571036 |
| chr14 | 63653198 | 63653248 |
| chr14 | 64305800 | 64305850 |
| chr14 | 66089400 | 66089450 |
| chr14 | 66244791 | 66244841 |
| chr14 | 66245243 | 66245293 |
| chr14 | 68680518 | 68680568 |
| chr14 | 68873746 | 68873796 |
| chr14 | 69322721 | 69322771 |
| chr10 | 51779035 | 51779085 |
| chr14 | 71689608 | 71689658 |
| chr14 | 78958170 | 78958220 |
| chr14 | 79558200 | 79558250 |
| chr14 | 81098904 | 81098954 |
| chr14 | 82314936 | 82314986 |
| chr14 | 87273683 | 87273733 |
| chr14 | 88513881 | 88513931 |
| chr10 | 54680577 | 54680627 |
| chr14 | 89992937 | 89992987 |
| chr14 | 92543604 | 92543654 |
| chr14 | 93824686 | 93824736 |
| chr10 | 56326610 | 56326660 |
| chr14 | 95252876 | 95252926 |
| chr14 | 95679620 | 95679670 |
| chr14 | 98423240 | 98423290 |
| chr14 | 101802263 | 101802313 |
| chr14 | 102527138 | 102527188 |
| chr14 | 102652806 | 102652856 |
| chr10 | 2221657 | 2221707 |
| chr14 | 102957354 | 102957404 |
| chr14 | 103066584 | 103066634 |
| chr14 | 104099626 | 104099676 |
| chr14 | 104358675 | 104358725 |
| chr14 | 104767669 | 104767719 |
| chr14 | 105231481 | 105231531 |
| chr15 | 20488360 | 20488410 |
| chr15 | 21269772 | 21269822 |
| chr15 | 21271178 | 21271228 |
| chr10 | 61977266 | 61977316 |
| chr15 | 28911720 | 28911770 |
| chr15 | 29097759 | 29097809 |
| chr15 | 29098150 | 29098200 |
| chr15 | 32273533 | 32273583 |
| chr15 | 33444412 | 33444462 |
| chr15 | 34592468 | 34592518 |
| chr15 | 37753331 | 37753381 |
| chr15 | 39804196 | 39804246 |
| chr15 | 40792929 | 40792979 |
| chr15 | 40946542 | 40946592 |
| chr15 | 41227887 | 41227937 |
| chr15 | 41886567 | 41886617 |
| chr15 | 42921959 | 42922009 |
| chr15 | 43844288 | 43844338 |
| chr15 | 43912895 | 43912945 |
| chr15 | 47811049 | 47811099 |
| chr15 | 48154585 | 48154635 |
| chr15 | 48161872 | 48161922 |
| chr15 | 49086008 | 49086058 |
| chr10 | 64661455 | 64661505 |
| chr15 | 49992843 | 49992893 |
| chr15 | 50125464 | 50125514 |
| chr15 | 53821616 | 53821666 |
| chr15 | 54460645 | 54460695 |
| chr15 | 54587507 | 54587557 |
| chr10 | 67240546 | 67240596 |
| chr15 | 65526366 | 65526416 |
| chr10 | 68551564 | 68551614 |
| chr15 | 68798226 | 68798276 |
| chr15 | 72992071 | 72992121 |
| chr15 | 73183032 | 73183082 |
| chr10 | 69228407 | 69228457 |
| chr15 | 79122770 | 79122820 |
| chr15 | 81849256 | 81849306 |
| chr15 | 83729756 | 83729806 |
| chr15 | 86897975 | 86898025 |
| chr15 | 87806034 | 87806084 |
| chr15 | 89679408 | 89679458 |
| chr10 | 69773072 | 69773122 |
| chr10 | 2891965 | 2892015 |
| chr10 | 69957663 | 69957713 |
| chr10 | 69957940 | 69957990 |
| chr15 | 99627216 | 99627266 |
| chr15 | 99626942 | 99626992 |
| chr15 | 99846757 | 99846807 |
| chr16 | 294072 | 294122 |
| chr16 | 2993567 | 2993617 |
| chr16 | 3994014 | 3994064 |
| chr16 | 4161040 | 4161090 |
| chr16 | 4408762 | 4408812 |
| chr16 | 4408662 | 4408712 |
| chr16 | 5085809 | 5085859 |
| chr16 | 5159218 | 5159268 |
| chr16 | 5794972 | 5795022 |
| chr16 | 6395243 | 6395293 |
| chr16 | 8605259 | 8605309 |
| chr16 | 9535599 | 9535649 |
| chr10 | 71563404 | 71563454 |
| chr10 | 71563622 | 71563672 |
| chr10 | 71563827 | 71563877 |
| chr16 | 16677540 | 16677590 |
| chr16 | 17329171 | 17329221 |
| chr16 | 21553583 | 21553633 |
| chr16 | 23326355 | 23326405 |
| chr16 | 23652259 | 23652309 |
| chr16 | 23652053 | 23652103 |
| chr16 | 24500904 | 24500954 |
| chr16 | 25275696 | 25275746 |
| chr16 | 25345931 | 25345981 |
| chr16 | 27121443 | 27121493 |
| chr16 | 27355940 | 27355990 |
| chr16 | 28997042 | 28997092 |
| chr16 | 29341255 | 29341305 |
| chr16 | 29804198 | 29804248 |
| chr16 | 30153978 | 30154028 |
| chr16 | 31968876 | 31968926 |
| chr16 | 32431207 | 32431257 |
| chr16 | 47955794 | 47955844 |
| chr16 | 47955886 | 47955936 |
| chr16 | 48744432 | 48744482 |
| chr16 | 53331985 | 53332035 |
| chr10 | 73135375 | 73135425 |
| chr16 | 54790634 | 54790684 |
| chr16 | 57422028 | 57422078 |
| chr16 | 60436687 | 60436737 |
| chr16 | 60719023 | 60719073 |
| chr16 | 61066278 | 61066328 |
| chr16 | 62533813 | 62533863 |
| chr16 | 62535158 | 62535208 |
| chr16 | 63597178 | 63597228 |
| chr16 | 65530260 | 65530310 |
| chr16 | 67841247 | 67841297 |
| chr16 | 68755477 | 68755527 |
| chr16 | 70251058 | 70251108 |
| chr16 | 74054463 | 74054513 |
| chr16 | 74125051 | 74125101 |
| chr16 | 77728239 | 77728289 |
| chr16 | 78599619 | 78599669 |
| chr16 | 81145331 | 81145381 |
| chr16 | 83888882 | 83888932 |
| chr16 | 83888801 | 83888851 |
| chr16 | 84058635 | 84058685 |
| chr16 | 84298435 | 84298485 |
| chr16 | 84544601 | 84544651 |
| chr10 | 3319173 | 3319223 |
| chr16 | 85707442 | 85707492 |
| chr16 | 86205946 | 86205996 |
| chr16 | 86435063 | 86435113 |
| chr16 | 88082341 | 88082391 |
| chr16 | 88376360 | 88376410 |
| chr16 | 88661183 | 88661233 |
| chr16 | 89019672 | 89019722 |
| chr16 | 89108789 | 89108839 |
| chr16 | 89859686 | 89859736 |
| chr17 | 1690348 | 1690398 |
| chr17 | 3139463 | 3139513 |
| chr17 | 3634038 | 3634088 |
| chr17 | 4632158 | 4632208 |
| chr17 | 5019479 | 5019529 |
| chr17 | 5591913 | 5591963 |
| chr17 | 6528819 | 6528869 |
| chr17 | 6529802 | 6529852 |
| chr17 | 6812177 | 6812227 |
| chr17 | 7045807 | 7045857 |
| chr17 | 7082884 | 7082934 |
| chr17 | 7248036 | 7248086 |
| chr17 | 7556256 | 7556306 |
| chr17 | 7631704 | 7631754 |
| chr17 | 9821572 | 9821622 |
| chr17 | 9842486 | 9842536 |
| chr17 | 9950959 | 9951009 |
| chr17 | 10074695 | 10074745 |
| chr17 | 10238641 | 10238691 |
| chr17 | 14852141 | 14852191 |
| chr17 | 17654261 | 17654311 |
| chr17 | 19801094 | 19801144 |
| chr10 | 80053063 | 80053113 |
| chr17 | 28120190 | 28120240 |
| chr17 | 28258819 | 28258869 |
| chr17 | 28278019 | 28278069 |
| chr17 | 30393270 | 30393320 |
| chr17 | 36831448 | 36831498 |
| chr10 | 81563691 | 81563741 |

Table 15CQ. Repeat Category MER63A

| | | |
|---|---|---|
| chr17 | 38344004 | 38344054 |
| chr18 | 27998944 | 27998994 |
| chr18 | 54338110 | 54338160 |
| chr18 | 58938483 | 58938533 |
| chr18 | 59367070 | 59367120 |
| chr18 | 62321171 | 62321221 |
| chr19 | 438082 | 438132 |
| chr19 | 5727319 | 5727369 |
| chr19 | 14292747 | 14292797 |
| chr19 | 40384870 | 40384920 |
| chr19 | 60145473 | 60145523 |
| chr19 | 61673912 | 61673962 |
| chr19 | 61673765 | 61673815 |
| chr1 | 13900746 | 13900796 |
| chr1 | 17395065 | 17395115 |
| chr1 | 21945039 | 21945089 |
| chr1 | 24033094 | 24033144 |
| chr1 | 39601783 | 39601833 |
| chr10 | 124286273 | 124286323 |
| chr1 | 90821623 | 90821673 |
| chr1 | 113615663 | 113615713 |
| chr1 | 147965385 | 147965435 |
| chr1 | 154089424 | 154089474 |
| chr1 | 201953707 | 201953757 |
| chr1 | 201953594 | 201953644 |
| chr1 | 208377402 | 208377452 |
| chr1 | 217611296 | 217611346 |
| chr1 | 231333978 | 231334028 |
| chr20 | 1639037 | 1639087 |
| chr20 | 4794544 | 4794594 |
| chr20 | 19256468 | 19256518 |
| chr20 | 25016578 | 25016628 |
| chr21 | 31772150 | 31772200 |
| chr21 | 40375173 | 40375223 |
| chr22 | 23950631 | 23950681 |
| chr11 | 2006284 | 2006334 |
| chr22 | 41890927 | 41890977 |
| chrX | 11555492 | 11555542 |
| chrX | 38684803 | 38684853 |
| chrX | 94539704 | 94539754 |
| chrX | 121777001 | 121777051 |
| chr2 | 3145169 | 3145219 |
| chr2 | 8708283 | 8708333 |
| chr2 | 39593484 | 39593534 |
| chr2 | 61902549 | 61902599 |
| chr2 | 64108613 | 64108663 |
| chr11 | 28567205 | 28567255 |
| chr2 | 203095986 | 203096036 |
| chr2 | 206690687 | 206690737 |
| chr2 | 207618146 | 207618196 |
| chr2 | 230717019 | 230717069 |
| chr2 | 240638557 | 240638607 |
| chr3 | 33972706 | 33972756 |
| chr3 | 35786197 | 35786247 |
| chr3 | 35786085 | 35786135 |
| chr3 | 53172979 | 53173029 |
| chr3 | 62349113 | 62349163 |
| chr3 | 65768491 | 65768541 |
| chr3 | 114617897 | 114617947 |
| chr3 | 128126644 | 128126694 |
| chr3 | 133026334 | 133026384 |
| chr3 | 190263915 | 190263965 |
| chr3 | 191615997 | 191616047 |
| chr4 | 5629895 | 5629945 |
| chr4 | 6429501 | 6429551 |
| chr4 | 46426251 | 46426301 |
| chr4 | 77296608 | 77296658 |
| chr4 | 110762298 | 110762348 |
| chr4 | 158890212 | 158890262 |
| chr4 | 167213535 | 167213585 |
| chr4 | 188362110 | 188362160 |
| chr4 | 188362485 | 188362535 |
| chr4 | 188362567 | 188362617 |
| chr4 | 191221781 | 191221831 |
| chr11 | 69591881 | 69591931 |
| chr11 | 69591678 | 69591728 |
| chr5 | 53204858 | 53204908 |
| chr5 | 58207802 | 58207852 |
| chr5 | 80159780 | 80159830 |
| chr5 | 157928299 | 157928349 |
| chr5 | 157978767 | 157978817 |
| chr5 | 172017491 | 172017541 |
| chr5 | 174009991 | 174010041 |
| chr5 | 180600261 | 180600311 |
| chr6 | 37008654 | 37008704 |
| chr6 | 42178912 | 42178962 |
| chr11 | 94557072 | 94557122 |
| chr11 | 94857374 | 94857424 |
| chr6 | 123110279 | 123110329 |
| chr7 | 4279874 | 4279924 |
| chr11 | 111790318 | 111790368 |
| chr7 | 67220930 | 67220980 |
| chr7 | 120032332 | 120032382 |
| chr7 | 124669591 | 124669641 |
| chr7 | 157682380 | 157682430 |
| chr8 | 21771256 | 21771306 |
| chr8 | 139246315 | 139246365 |
| chr9 | 3891195 | 3891245 |
| chr9 | 3891098 | 3891148 |
| chr9 | 68289256 | 68289306 |
| chr9 | 93525460 | 93525510 |
| chr9 | 130013737 | 130013787 |
| chr9 | 131908284 | 131908334 |
| chr9 | 135389171 | 135389221 |
| chr10 | 21769230 | 21769280 |
| chr12 | 6080881 | 6080931 |
| chr12 | 6822395 | 6822445 |
| chr12 | 25861147 | 25861197 |
| chr12 | 49631606 | 49631656 |
| chr10 | 29300672 | 29300722 |
| chr12 | 103917345 | 103917395 |
| chr12 | 119820254 | 119820304 |
| chr12 | 128390825 | 128390875 |
| chr13 | 22078920 | 22078970 |
| chr10 | 36624838 | 36624888 |
| chr13 | 29949747 | 29949797 |
| chr14 | 22748871 | 22748921 |
| chr10 | 50093323 | 50093373 |
| chr14 | 61435048 | 61435098 |
| chr10 | 61977266 | 61977316 |
| chr15 | 32273533 | 32273583 |
| chr15 | 39804196 | 39804246 |
| chr15 | 49992843 | 49992893 |
| chr16 | 5794972 | 5795022 |
| chr16 | 25275696 | 25275746 |
| chr16 | 84058635 | 84058685 |
| chr17 | 9842486 | 9842536 |

Table 15CR. Repeat Category MER66C

| | | |
|---|---|---|
| chr17 | 78847153 | 78847203 |
| chr10 | 4545080 | 4545130 |
| chr19 | 7721357 | 7721407 |
| chr19 | 7722794 | 7722844 |
| chr19 | 18156781 | 18156831 |
| chr19 | 44229185 | 44229235 |
| chr19 | 56094980 | 56095030 |
| chr1 | 82665820 | 82665870 |
| chr1 | 173925499 | 173925549 |
| chr1 | 206210941 | 206210991 |
| chr1 | 228456118 | 228456168 |
| chr1 | 241661772 | 241661822 |
| chr20 | 2422840 | 2422890 |
| chr20 | 17886196 | 17886246 |
| chr20 | 32753740 | 32753790 |
| chrX | 51019934 | 51019984 |
| chr2 | 13234301 | 13234351 |
| chr2 | 76646860 | 76646910 |
| chr2 | 97206283 | 97206333 |
| chr2 | 188021552 | 188021602 |
| chr11 | 46409574 | 46409624 |
| chr3 | 15394903 | 15394953 |
| chr3 | 52753970 | 52754020 |
| chr3 | 130110962 | 130111012 |
| chr3 | 147960623 | 147960673 |
| chr4 | 5591448 | 5591498 |
| chr4 | 5591361 | 5591411 |
| chr4 | 46782153 | 46782203 |
| chr4 | 72162987 | 72163037 |
| chr4 | 185333536 | 185333586 |
| chr4 | 189808416 | 189808466 |
| chr5 | 1428571 | 1428621 |
| chr11 | 105728036 | 105728086 |
| chr7 | 55418979 | 55419029 |
| chr7 | 72954538 | 72954588 |
| chr7 | 147615956 | 147616006 |
| chr8 | 34494590 | 34494640 |
| chr8 | 40081174 | 40081224 |
| chr9 | 19108028 | 19108078 |
| chr9 | 26351897 | 26351947 |
| chr11 | 133253322 | 133253372 |
| chr11 | 133252830 | 133252880 |
| chr9 | 67879966 | 67880016 |
| chr9 | 67882339 | 67882389 |
| chr10 | 23673113 | 23673163 |
| chr12 | 47824452 | 47824502 |
| chr13 | 47945447 | 47945497 |
| chr13 | 48199533 | 48199583 |
| chr13 | 99044495 | 99044545 |
| chr16 | 63597178 | 63597228 |
| chr16 | 89019672 | 89019722 |
| chr17 | 5019479 | 5019529 |
| chr17 | 6528819 | 6528869 |
| chr17 | 7248036 | 7248086 |

Table 15CS. Repeat Category MER67A

| | | |
|---|---|---|
| chr17 | 55383852 | 55383902 |
| chr18 | 42958118 | 42958168 |
| chr1 | 239052461 | 239052511 |
| chr1 | 245891792 | 245891842 |
| chr22 | 18306254 | 18306304 |
| chr22 | 18308023 | 18308073 |
| chr22 | 18313807 | 18313857 |
| chr22 | 34118405 | 34118455 |
| chr22 | 46581440 | 46581490 |
| chr22 | 48519005 | 48519055 |
| chr2 | 61079326 | 61079376 |
| chr4 | 43882990 | 43883040 |
| chr4 | 44591443 | 44591493 |
| chr4 | 69226257 | 69226307 |
| chr4 | 77304481 | 77304531 |
| chr4 | 166532817 | 166532867 |
| chr4 | 176200658 | 176200708 |
| chr4 | 188984139 | 188984189 |
| chr5 | 105996596 | 105996646 |
| chr5 | 178379857 | 178379907 |
| chr6 | 26425471 | 26425521 |
| chr6 | 27202998 | 27203048 |
| chr6 | 37946584 | 37946634 |
| chr7 | 4748760 | 4748810 |
| chr7 | 20088989 | 20089039 |
| chr7 | 51835806 | 51835856 |
| chr8 | 27770505 | 27770555 |
| chr8 | 59625641 | 59625691 |
| chr9 | 34147287 | 34147337 |
| chr9 | 113598091 | 113598141 |
| chr13 | 18187302 | 18187352 |
| chr13 | 80882686 | 80882736 |
| chr14 | 43582701 | 43582751 |
| chr10 | 49660196 | 49660246 |
| chr16 | 17329171 | 17329221 |
| chr10 | 73135375 | 73135425 |
| chr16 | 62533813 | 62533863 |
| chr16 | 62535158 | 62535208 |
| chr17 | 14852141 | 14852191 |

Table 15CT. Repeat Category MER67D

| | | |
|---|---|---|
| chr17 | 50076663 | 50076713 |
| chr17 | 50076545 | 50076595 |
| chr18 | 26200976 | 26201026 |
| chr18 | 56314587 | 56314637 |
| chr18 | 56665314 | 56665364 |
| chr18 | 65443448 | 65443498 |
| chr18 | 69857096 | 69857146 |
| chr18 | 72895069 | 72895119 |
| chr18 | 72894895 | 72894945 |
| chr10 | 109025149 | 109025199 |
| chr1 | 191824637 | 191824687 |
| chr1 | 192481463 | 192481513 |
| chr20 | 42661734 | 42661784 |
| chr21 | 46405158 | 46405208 |
| chr22 | 44216198 | 44216248 |
| chrX | 28582994 | 28583044 |
| chr2 | 721783 | 721833 |
| chr2 | 52641697 | 52641747 |
| chr2 | 94875133 | 94875183 |
| chr2 | 129760002 | 129760052 |
| chr2 | 130830586 | 130830636 |
| chr2 | 132332374 | 132332424 |
| chr2 | 187397091 | 187397141 |
| chr11 | 39455224 | 39455274 |
| chr4 | 43458321 | 43458371 |
| chr4 | 133498126 | 133498176 |
| chr4 | 135529961 | 135530011 |
| chr4 | 162228125 | 162228175 |
| chr4 | 163496714 | 163496764 |
| chr4 | 171947817 | 171947867 |
| chr5 | 25430145 | 25430195 |
| chr5 | 28748974 | 28749024 |
| chr5 | 29341077 | 29341127 |
| chr5 | 29342489 | 29342539 |
| chr6 | 116186093 | 116186143 |
| chr6 | 142031461 | 142031511 |
| chr11 | 105740918 | 105740968 |
| chr7 | 66893714 | 66893764 |
| chr8 | 1300077 | 1300127 |
| chr8 | 87038216 | 87038266 |
| chr8 | 137401134 | 137401184 |
| chr9 | 12449787 | 12449837 |
| chr9 | 120674529 | 120674579 |
| chr12 | 40613466 | 40613516 |
| chr12 | 126782831 | 126782881 |
| chr13 | 62898016 | 62898066 |
| chr13 | 80970219 | 80970269 |
| chr13 | 85093609 | 85093659 |
| chr13 | 86267888 | 86267938 |

Table 15CU. Repeat Category MER6A

| | | |
|---|---|---|
| chr17 | 55371841 | 55371891 |
| chr18 | 18708053 | 18708103 |
| chr18 | 58690757 | 58690807 |
| chr19 | 50363451 | 50363501 |
| chr19 | 50363638 | 50363688 |
| chr1 | 185754121 | 185754171 |
| chr1 | 187276631 | 187276681 |
| chr1 | 197896466 | 197896516 |
| chr1 | 228550874 | 228550924 |
| chr20 | 6039693 | 6039743 |
| chr21 | 16298966 | 16299016 |
| chr21 | 37224689 | 37224739 |
| chr21 | 41936038 | 41936088 |
| chr22 | 30222092 | 30222142 |
| chr10 | 9924602 | 9924652 |
| chr11 | 3994157 | 3994207 |
| chrX | 24011092 | 24011142 |
| chrX | 24013248 | 24013298 |
| chrX | 30468831 | 30468881 |
| chrX | 33851654 | 33851704 |
| chr2 | 9534966 | 9535016 |
| chr2 | 54881521 | 54881571 |
| chr2 | 65267889 | 65267939 |
| chr2 | 86160451 | 86160501 |
| chr2 | 97944141 | 97944191 |
| chr2 | 107773049 | 107773099 |
| chr2 | 107772966 | 107773016 |
| chr2 | 127420107 | 127420157 |
| chr2 | 132110033 | 132110083 |
| chr2 | 197701075 | 197701125 |
| chr2 | 199045946 | 199045996 |
| chr2 | 236793626 | 236793676 |
| chr3 | 5078949 | 5078999 |
| chr3 | 5079989 | 5080039 |
| chr3 | 13528671 | 13528721 |
| chr3 | 19251241 | 19251291 |
| chr11 | 55228251 | 55228301 |
| chr3 | 70462822 | 70462872 |
| chr3 | 85438381 | 85438431 |
| chr3 | 175744173 | 175744223 |
| chr3 | 195486668 | 195486718 |
| chr4 | 6083158 | 6083208 |
| chr4 | 38617257 | 38617307 |
| chr4 | 98591577 | 98591627 |
| chr4 | 182182875 | 182182925 |
| chr4 | 185170015 | 185170065 |
| chr4 | 185171342 | 185171392 |
| chr4 | 185171569 | 185171619 |
| chr5 | 698725 | 698775 |
| chr5 | 3875646 | 3875696 |
| chr5 | 52894591 | 52894641 |
| chr5 | 55743123 | 55743173 |
| chr6 | 10913493 | 10913543 |
| chr6 | 20553687 | 20553737 |
| chr6 | 66948363 | 66948413 |
| chr6 | 106398279 | 106398329 |
| chr6 | 136892550 | 136892600 |
| chr6 | 159382256 | 159382306 |
| chr6 | 167038839 | 167038889 |
| chr6 | 167039107 | 167039157 |
| chr6 | 167039337 | 167039387 |
| chr6 | 168410745 | 168410795 |
| chr7 | 50868184 | 50868234 |
| chr7 | 127787213 | 127787263 |
| chr7 | 143748226 | 143748276 |
| chr8 | 142130704 | 142130754 |
| chr11 | 130353664 | 130353714 |
| chr9 | 3915610 | 3915660 |
| chr9 | 9701044 | 9701094 |
| chr9 | 32909794 | 32909844 |
| chr12 | 7148199 | 7148249 |
| chr12 | 68865794 | 68865844 |
| chr12 | 106812813 | 106812863 |
| chr14 | 53514748 | 53514798 |
| chr14 | 53515273 | 53515323 |
| chr14 | 79558200 | 79558250 |
| chr14 | 89992937 | 89992987 |
| chr15 | 37753331 | 37753381 |
| chr15 | 40946542 | 40946592 |
| chr15 | 68798226 | 68798276 |
| chr15 | 99846757 | 99846807 |
| chr16 | 24500904 | 24500954 |
| chr16 | 54790634 | 54790684 |
| chr17 | 6812177 | 6812227 |
| chr17 | 9821572 | 9821622 |

Table 15CV. Repeat Category MER70A

| | | |
|---|---|---|
| chr17 | 49028352 | 49028402 |
| chr17 | 49029549 | 49029599 |
| chr19 | 2844874 | 2844924 |
| chr19 | 36206063 | 36206113 |
| chr1 | 214702420 | 214702470 |
| chr1 | 244143609 | 244143659 |
| chr20 | 4178279 | 4178329 |
| chr20 | 41029425 | 41029475 |
| chr21 | 22190325 | 22190375 |
| chr21 | 22392827 | 22392877 |
| chr21 | 22392663 | 22392713 |
| chrX | 15167186 | 15167236 |
| chr2 | 65878198 | 65878248 |
| chr2 | 89187161 | 89187211 |
| chr2 | 90025987 | 90026037 |
| chr2 | 140817822 | 140817872 |
| chr2 | 211944927 | 211944977 |
| chr2 | 232490643 | 232490693 |
| chr4 | 64886141 | 64886191 |
| chr4 | 117912241 | 117912291 |
| chr5 | 36079493 | 36079543 |
| chr11 | 70188073 | 70188123 |
| chr5 | 111643930 | 111643980 |
| chr6 | 2748663 | 2748713 |
| chr6 | 2750521 | 2750571 |
| chr11 | 88029680 | 88029730 |
| chr6 | 78646575 | 78646625 |
| chr6 | 156712221 | 156712271 |
| chr11 | 103636850 | 103636900 |
| chr7 | 125278747 | 125278797 |
| chr9 | 95884785 | 95884835 |
| chr14 | 20441597 | 20441647 |
| chr14 | 22393930 | 22393980 |
| chr14 | 22393844 | 22393894 |
| chr14 | 72066958 | 72067008 |
| chr10 | 65702323 | 65702373 |
| chr16 | 18259831 | 18259881 |
| chr10 | 3305135 | 3305185 |
| chr10 | 3304915 | 3304965 |

Table 15CW. Repeat Category MER91C

| | | |
|---|---|---|
| chr17 | 79249366 | 79249416 |
| chr18 | 27283995 | 27284045 |
| chr10 | 102278766 | 102278816 |
| chr19 | 10423266 | 10423316 |
| chr19 | 14546057 | 14546107 |
| chr1 | 40709068 | 40709118 |
| chr1 | 40708963 | 40709013 |
| chr1 | 84418440 | 84418490 |
| chr1 | 114306751 | 114306801 |
| chr1 | 218042807 | 218042857 |
| chr20 | 49748527 | 49748577 |
| chr21 | 26224437 | 26224487 |
| chr21 | 41440257 | 41440307 |
| chr22 | 15922229 | 15922279 |
| chr22 | 44257847 | 44257897 |
| chrX | 39189265 | 39189315 |
| chrX | 128819807 | 128819857 |
| chr2 | 234021660 | 234021710 |
| chr3 | 64717860 | 64717910 |
| chr3 | 161350757 | 161350807 |
| chr4 | 47647420 | 47647470 |
| chr11 | 66678301 | 66678351 |
| chr4 | 148700551 | 148700601 |
| chr5 | 75094719 | 75094769 |
| chr5 | 75094792 | 75094842 |
| chr6 | 90326547 | 90326597 |
| chr11 | 96096607 | 96096657 |
| chr6 | 131090917 | 131090967 |
| chr6 | 167338450 | 167338500 |
| chr6 | 167338363 | 167338413 |
| chr11 | 118942385 | 118942435 |
| chr11 | 119877086 | 119877136 |
| chr9 | 77759196 | 77759246 |
| chr9 | 123956146 | 123956196 |
| chr9 | 127839512 | 127839562 |
| chr12 | 88291980 | 88292030 |
| chr12 | 93060962 | 93061012 |
| chr14 | 89034619 | 89034669 |
| chr14 | 89034471 | 89034521 |
| chr14 | 91414691 | 91414741 |
| chr15 | 72117868 | 72117918 |
| chr15 | 73334369 | 73334419 |
| chr16 | 23296035 | 23296085 |
| chr16 | 84369434 | 84369484 |

Table 15CX. Repeat Category MER92B

| | | |
|---|---|---|
| chr18 | 60093475 | 60093525 |
| chr19 | 11573890 | 11573940 |
| chr19 | 12261802 | 12261852 |
| chr19 | 12331811 | 12331861 |
| chr19 | 12518151 | 12518201 |
| chr19 | 19874828 | 19874878 |
| chr19 | 19875997 | 19876047 |
| chr19 | 20141234 | 20141284 |
| chr19 | 20142444 | 20142494 |
| chr19 | 20397413 | 20397463 |
| chr19 | 20999109 | 20999159 |
| chr19 | 21120161 | 21120211 |
| chr19 | 23767442 | 23767492 |
| chr19 | 24009324 | 24009374 |
| chr19 | 58196749 | 58196799 |
| chr1 | 3685744 | 3685794 |
| chr1 | 244186563 | 244186613 |
| chr1 | 244392331 | 244392381 |
| chr20 | 23180294 | 23180344 |
| chr20 | 23284166 | 23284216 |
| chr20 | 36262386 | 36262436 |
| chr20 | 38227723 | 38227773 |
| chr20 | 60213963 | 60214013 |
| chr21 | 30166800 | 30166850 |
| chr21 | 38252030 | 38252080 |
| chr21 | 41843126 | 41843176 |
| chr21 | 43255981 | 43256031 |
| chr21 | 43381929 | 43381979 |
| chr21 | 43509824 | 43509874 |
| chr10 | 601716 | 601766 |
| chrX | 141389174 | 141389224 |
| chrX | 141389603 | 141389653 |
| chrX | 150782676 | 150782726 |
| chrY | 27454781 | 27454831 |
| chr2 | 627506 | 627556 |
| chr2 | 24137717 | 24137767 |
| chr2 | 24137533 | 24137583 |
| chr11 | 24703597 | 24703647 |
| chr2 | 95550831 | 95550881 |
| chr2 | 96158888 | 96158938 |
| chr2 | 127802962 | 127803012 |
| chr2 | 198432701 | 198432751 |
| chr2 | 199673167 | 199673217 |
| chr2 | 230690606 | 230690656 |
| chr3 | 11287847 | 11287897 |
| chr3 | 149325158 | 149325208 |
| chr3 | 181791360 | 181791410 |
| chr4 | 4404190 | 4404240 |
| chr4 | 87847349 | 87847399 |
| chr4 | 118913897 | 118913947 |
| chr6 | 80475146 | 80475196 |
| chr6 | 154120105 | 154120155 |
| chr6 | 164999271 | 164999321 |
| chr11 | 107269280 | 107269330 |
| chr7 | 2995204 | 2995254 |
| chr7 | 34434499 | 34434549 |
| chr7 | 63340085 | 63340135 |
| chr7 | 64254638 | 64254688 |
| chr7 | 98047417 | 98047467 |
| chr8 | 32825100 | 32825150 |
| chr8 | 143270260 | 143270310 |
| chr8 | 143270046 | 143270096 |
| chr9 | 74527285 | 74527335 |
| chr12 | 31673502 | 31673552 |
| chr12 | 127831449 | 127831499 |
| chr13 | 53762636 | 53762686 |
| chr13 | 101454108 | 101454158 |
| chr13 | 104664019 | 104664069 |
| chr14 | 102175034 | 102175084 |
| chr14 | 102761115 | 102761165 |
| chr15 | 67656495 | 67656545 |
| chr16 | 87637388 | 87637438 |

Table 15CY. Repeat Category MLT1A

| | | |
|---|---|---|
| chr10 | 81611942 | 81611992 |
| chr10 | 81975981 | 81976031 |
| chr17 | 42389181 | 42389231 |
| chr10 | 82047624 | 82047674 |
| chr17 | 44792287 | 44792337 |
| chr17 | 45652048 | 45652098 |
| chr10 | 83225068 | 83225118 |
| chr17 | 46976620 | 46976670 |
| chr17 | 47822557 | 47822607 |
| chr17 | 49571045 | 49571095 |
| chr17 | 53252158 | 53252208 |
| chr17 | 55581142 | 55581192 |
| chr17 | 60775221 | 60775271 |
| chr17 | 61210321 | 61210371 |
| chr17 | 62949457 | 62949507 |
| chr17 | 64026522 | 64026572 |
| chr17 | 66826329 | 66826379 |
| chr17 | 66954372 | 66954422 |
| chr17 | 68401794 | 68401844 |
| chr17 | 70642640 | 70642690 |
| chr17 | 71736042 | 71736092 |
| chr17 | 72359287 | 72359337 |
| chr17 | 72531357 | 72531407 |
| chr17 | 73223441 | 73223491 |
| chr17 | 73232506 | 73232556 |
| chr10 | 88661731 | 88661781 |
| chr10 | 88757703 | 88757753 |
| chr17 | 75363032 | 75363082 |
| chr17 | 75443452 | 75443502 |
| chr17 | 75613512 | 75613562 |
| chr17 | 76177819 | 76177869 |
| chr17 | 76889972 | 76890022 |
| chr17 | 76903220 | 76903270 |
| chr17 | 77230554 | 77230604 |
| chr17 | 77230477 | 77230527 |
| chr17 | 78579280 | 78579330 |
| chr17 | 79310647 | 79310697 |
| chr17 | 79652646 | 79652696 |
| chr10 | 93320659 | 93320709 |
| chr17 | 81683706 | 81683756 |
| chr18 | 2954315 | 2954365 |
| chr18 | 3519165 | 3519215 |
| chr18 | 3518978 | 3519028 |
| chr18 | 3608626 | 3608676 |
| chr18 | 4559148 | 4559198 |
| chr18 | 4959385 | 4959435 |
| chr18 | 5017849 | 5017899 |
| chr18 | 5038026 | 5038076 |
| chr18 | 7128794 | 7128844 |
| chr18 | 7341710 | 7341760 |
| chr18 | 7381839 | 7381889 |
| chr18 | 8716666 | 8716716 |
| chr18 | 10647798 | 10647848 |
| chr10 | 94722546 | 94722596 |
| chr18 | 13833454 | 13833504 |
| chr18 | 13935237 | 13935287 |
| chr18 | 17539715 | 17539765 |
| chr18 | 17913844 | 17913894 |
| chr18 | 18628985 | 18629035 |
| chr18 | 19016706 | 19016756 |
| chr18 | 19236242 | 19236292 |
| chr18 | 19423005 | 19423055 |
| chr18 | 20671551 | 20671601 |
| chr18 | 21836914 | 21836964 |
| chr10 | 95605602 | 95605652 |
| chr18 | 22474861 | 22474911 |
| chr10 | 95760596 | 95760646 |
| chr18 | 25074663 | 25074713 |
| chr10 | 96724400 | 96724450 |
| chr18 | 38348176 | 38348226 |
| chr18 | 45244783 | 45244833 |
| chr18 | 46493576 | 46493626 |
| chr18 | 46493517 | 46493567 |
| chr18 | 47518863 | 47518913 |
| chr18 | 48923753 | 48923803 |
| chr18 | 49828173 | 49828223 |
| chr18 | 51774084 | 51774134 |
| chr18 | 53456341 | 53456391 |
| chr18 | 57656031 | 57656081 |
| chr10 | 98244194 | 98244244 |
| chr18 | 60409439 | 60409489 |
| chr18 | 63126120 | 63126170 |
| chr18 | 63723225 | 63723275 |
| chr18 | 64694846 | 64694896 |
| chr18 | 65801419 | 65801469 |
| chr18 | 66218240 | 66218290 |
| chr18 | 67167446 | 67167496 |
| chr18 | 67364975 | 67365025 |
| chr18 | 67364821 | 67364871 |
| chr18 | 67564979 | 67565029 |
| chr18 | 68015471 | 68015521 |
| chr18 | 68594111 | 68594161 |
| chr18 | 68794859 | 68794909 |
| chr18 | 69892343 | 69892393 |
| chr18 | 70215324 | 70215374 |
| chr18 | 70615032 | 70615082 |
| chr18 | 71939462 | 71939512 |
| chr18 | 74012476 | 74012526 |
| chr10 | 98994548 | 98994598 |
| chr18 | 74786938 | 74786988 |
| chr18 | 74808282 | 74808332 |
| chr19 | 310891 | 310941 |
| chr19 | 2422245 | 2422295 |
| chr19 | 3056906 | 3056956 |
| chr19 | 5858131 | 5858181 |
| chr19 | 6385257 | 6385307 |
| chr10 | 102054584 | 102054634 |
| chr19 | 8759702 | 8759752 |
| chr19 | 10512264 | 10512314 |
| chr19 | 11687350 | 11687400 |
| chr19 | 11688645 | 11688695 |
| chr19 | 11726812 | 11726862 |
| chr19 | 12254092 | 12254142 |
| chr19 | 12533347 | 12533397 |
| chr19 | 13098307 | 13098357 |
| chr19 | 13275759 | 13275809 |
| chr19 | 13535918 | 13535968 |
| chr19 | 13606603 | 13606653 |
| chr19 | 14420236 | 14420286 |
| chr19 | 16646692 | 16646742 |
| chr19 | 16647855 | 16647905 |
| chr19 | 16758682 | 16758732 |
| chr19 | 16771851 | 16771901 |
| chr19 | 16771760 | 16771810 |
| chr19 | 16941728 | 16941778 |
| chr10 | 103319368 | 103319418 |
| chr10 | 103597586 | 103597636 |
| chr19 | 18790945 | 18790995 |
| chr19 | 34809321 | 34809371 |
| chr19 | 35909737 | 35909787 |
| chr19 | 36311614 | 36311664 |
| chr19 | 36311413 | 36311463 |
| chr19 | 37617490 | 37617540 |
| chr19 | 38965314 | 38965364 |
| chr19 | 45365521 | 45365571 |
| chr19 | 45365588 | 45365638 |
| chr19 | 45876484 | 45876534 |
| chr19 | 49121414 | 49121464 |
| chr19 | 49121314 | 49121364 |
| chr19 | 49178403 | 49178453 |
| chr10 | 107792814 | 107792864 |
| chr19 | 49361007 | 49361057 |
| chr19 | 49788468 | 49788518 |
| chr10 | 108184472 | 108184522 |
| chr10 | 108303197 | 108303247 |
| chr19 | 51362454 | 51362504 |
| chr10 | 110043280 | 110043330 |
| chr19 | 52149177 | 52149227 |
| chr19 | 52148809 | 52148859 |
| chr19 | 52481083 | 52481133 |
| chr19 | 53209375 | 53209425 |
| chr19 | 53434483 | 53434533 |
| chr10 | 112161847 | 112161897 |
| chr19 | 55268566 | 55268616 |
| chr19 | 56403657 | 56403707 |
| chr19 | 56930240 | 56930290 |
| chr19 | 57357731 | 57357781 |
| chr19 | 57678883 | 57678933 |
| chr19 | 57904690 | 57904740 |
| chr19 | 58129969 | 58130019 |
| chr19 | 61281807 | 61281857 |
| chr19 | 61348792 | 61348842 |
| chr19 | 61642757 | 61642807 |
| chr19 | 61880909 | 61880959 |
| chr19 | 62143366 | 62143416 |
| chr19 | 62176096 | 62176146 |
| chr19 | 62191428 | 62191478 |
| chr1 | 2422896 | 2422946 |
| chr1 | 3239901 | 3239951 |
| chr1 | 3508591 | 3508641 |
| chr1 | 4861387 | 4861437 |
| chr1 | 5617136 | 5617186 |
| chr1 | 6988998 | 6989048 |
| chr1 | 7026200 | 7026250 |
| chr10 | 116568218 | 116568268 |
| chr1 | 8611849 | 8611899 |
| chr1 | 11676008 | 11676058 |
| chr1 | 13170889 | 13170939 |
| chr10 | 118119298 | 118119348 |
| chr1 | 13329594 | 13329644 |
| chr1 | 13498819 | 13498869 |
| chr1 | 13509434 | 13509484 |
| chr1 | 13509267 | 13509317 |
| chr1 | 17194457 | 17194507 |
| chr1 | 18582376 | 18582426 |
| chr1 | 21199715 | 21199765 |
| chr1 | 22803523 | 22803573 |
| chr1 | 23457487 | 23457537 |
| chr10 | 120277329 | 120277379 |
| chr1 | 26264385 | 26264435 |
| chr10 | 6296629 | 6296679 |
| chr1 | 27573683 | 27573733 |
| chr1 | 31822718 | 31822768 |
| chr10 | 120915467 | 120915517 |
| chr1 | 35177854 | 35177904 |
| chr1 | 35705258 | 35705308 |
| chr10 | 121029097 | 121029147 |
| chr10 | 121188378 | 121188428 |
| chr1 | 38623270 | 38623320 |
| chr1 | 39916475 | 39916525 |
| chr1 | 42245641 | 42245691 |
| chr1 | 42598225 | 42598275 |
| chr1 | 42906324 | 42906374 |
| chr1 | 45126460 | 45126510 |
| chr1 | 50552740 | 50552790 |
| chr1 | 52401142 | 52401192 |
| chr1 | 53240211 | 53240261 |
| chr1 | 54347852 | 54347902 |
| chr1 | 55771665 | 55771715 |
| chr1 | 60438978 | 60439028 |
| chr1 | 63061742 | 63061792 |
| chr1 | 63172408 | 63172458 |
| chr1 | 63172931 | 63172981 |
| chr1 | 64956952 | 64957002 |
| chr1 | 65125223 | 65125273 |
| chr1 | 66943454 | 66943504 |
| chr1 | 67020448 | 67020498 |
| chr1 | 69999586 | 69999636 |
| chr1 | 78659185 | 78659235 |
| chr1 | 79154137 | 79154187 |
| chr1 | 79208612 | 79208662 |
| chr1 | 79237605 | 79237655 |
| chr1 | 81416608 | 81416658 |
| chr1 | 81484641 | 81484691 |
| chr1 | 82025148 | 82025198 |
| chr1 | 82298900 | 82298950 |
| chr1 | 83268350 | 83268400 |
| chr1 | 83577899 | 83577949 |
| chr1 | 87979260 | 87979310 |
| chr1 | 89491487 | 89491537 |
| chr1 | 89490800 | 89490850 |
| chr1 | 91971378 | 91971428 |
| chr1 | 92765587 | 92765637 |
| chr1 | 92923914 | 92923964 |
| chr1 | 92923775 | 92923825 |
| chr1 | 93360323 | 93360373 |
| chr1 | 93360776 | 93360826 |
| chr1 | 94948623 | 94948673 |
| chr1 | 97915599 | 97915649 |
| chr1 | 100110375 | 100110425 |
| chr1 | 103414281 | 103414331 |
| chr1 | 105056441 | 105056491 |
| chr10 | 125889077 | 125889127 |
| chr1 | 107820276 | 107820326 |
| chr1 | 109807413 | 109807463 |
| chr1 | 110287077 | 110287127 |
| chr1 | 112336971 | 112337021 |
| chr1 | 112340213 | 112340263 |
| chr1 | 112507004 | 112507054 |
| chr1 | 112828223 | 112828273 |
| chr1 | 112882515 | 112882565 |
| chr10 | 126253295 | 126253345 |
| chr1 | 115095593 | 115095643 |
| chr10 | 126349833 | 126349883 |
| chr10 | 126523450 | 126523500 |
| chr1 | 146651960 | 146652010 |
| chr1 | 146891346 | 146891396 |
| chr10 | 7179541 | 7179591 |
| chr1 | 151972137 | 151972187 |
| chr10 | 128186662 | 128186712 |
| chr1 | 157294714 | 157294764 |
| chr1 | 157979341 | 157979391 |
| chr1 | 158747332 | 158747382 |
| chr1 | 161204657 | 161204707 |
| chr1 | 162893018 | 162893068 |
| chr1 | 163017873 | 163017923 |
| chr1 | 163039703 | 163039753 |
| chr10 | 128667547 | 128667597 |
| chr1 | 171441372 | 171441422 |
| chr1 | 180967122 | 180967172 |
| chr1 | 181386209 | 181386259 |
| chr1 | 186091563 | 186091613 |
| chr1 | 188268696 | 188268746 |
| chr1 | 188619808 | 188619858 |
| chr1 | 188769788 | 188769838 |
| chr1 | 189100184 | 189100234 |
| chr1 | 191820693 | 191820743 |
| chr1 | 193859848 | 193859898 |
| chr1 | 195391224 | 195391274 |
| chr1 | 199404186 | 199404236 |
| chr1 | 201779602 | 201779652 |
| chr1 | 202793549 | 202793599 |
| chr10 | 130653697 | 130653747 |
| chr1 | 208450031 | 208450081 |
| chr1 | 208747164 | 208747214 |
| chr1 | 208747294 | 208747344 |
| chr1 | 209181441 | 209181491 |
| chr10 | 130770527 | 130770577 |
| chr1 | 211503770 | 211503820 |
| chr1 | 212924432 | 212924482 |
| chr1 | 215347907 | 215347957 |
| chr1 | 215461268 | 215461318 |
| chr1 | 217987457 | 217987507 |
| chr1 | 223789014 | 223789064 |
| chr1 | 225092592 | 225092642 |
| chr1 | 225613996 | 225614046 |
| chr1 | 228452409 | 228452459 |
| chr1 | 229158965 | 229159015 |
| chr1 | 229999961 | 230000011 |
| chr1 | 230253751 | 230253801 |
| chr1 | 231909179 | 231909229 |
| chr1 | 232149826 | 232149876 |
| chr1 | 234181411 | 234181461 |
| chr10 | 131784494 | 131784544 |
| chr1 | 234311698 | 234311748 |
| chr1 | 234693440 | 234693490 |
| chr10 | 131821853 | 131821903 |
| chr1 | 237502167 | 237502217 |
| chr1 | 237981750 | 237981800 |
| chr10 | 132168598 | 132168648 |
| chr1 | 241978987 | 241979037 |
| chr1 | 242240847 | 242240897 |
| chr1 | 242631008 | 242631058 |
| chr1 | 242914268 | 242914318 |
| chr1 | 243295197 | 243295247 |
| chr1 | 244499146 | 244499196 |
| chr1 | 244500202 | 244500252 |
| chr10 | 132476331 | 132476381 |
| chr20 | 1310001 | 1310051 |
| chr20 | 1309684 | 1309734 |
| chr20 | 1380176 | 1380226 |
| chr20 | 3456069 | 3456119 |
| chr20 | 4643710 | 4643760 |
| chr20 | 5693133 | 5693183 |
| chr10 | 132892314 | 132892364 |
| chr20 | 10419827 | 10419877 |
| chr20 | 17425983 | 17426033 |
| chr20 | 17484658 | 17484708 |
| chr20 | 18414129 | 18414179 |
| chr20 | 19056353 | 19056403 |
| chr20 | 19486777 | 19486827 |
| chr20 | 21785618 | 21785668 |
| chr20 | 25108238 | 25108288 |
| chr20 | 25214568 | 25214618 |
| chr20 | 25214677 | 25214727 |
| chr20 | 25410958 | 25411008 |
| chr20 | 33134289 | 33134339 |
| chr20 | 33555072 | 33555122 |
| chr10 | 133682067 | 133682117 |
| chr10 | 133733423 | 133733473 |
| chr20 | 38756863 | 38756913 |
| chr20 | 40242328 | 40242378 |
| chr20 | 41723146 | 41723196 |
| chr20 | 43128178 | 43128228 |
| chr20 | 44437862 | 44437912 |
| chr20 | 46202878 | 46202928 |
| chr10 | 133965188 | 133965238 |
| chr20 | 50312196 | 50312246 |
| chr20 | 51901415 | 51901465 |
| chr20 | 53441138 | 53441188 |
| chr20 | 56413968 | 56414018 |
| chr20 | 56413906 | 56413956 |
| chr20 | 56864773 | 56864823 |
| chr10 | 134384914 | 134384964 |
| chr20 | 61352702 | 61352752 |
| chr20 | 61559507 | 61559557 |
| chr20 | 62032798 | 62032848 |
| chr10 | 134647407 | 134647457 |
| chr20 | 62945179 | 62945229 |
| chr20 | 62974274 | 62974324 |
| chr20 | 62975462 | 62975512 |
| chr20 | 62993513 | 62993563 |
| chr20 | 63002219 | 63002269 |
| chr21 | 16313687 | 16313737 |
| chr21 | 17865156 | 17865206 |
| chr21 | 18496273 | 18496323 |
| chr21 | 19669574 | 19669624 |
| chr21 | 20057641 | 20057691 |
| chr21 | 21751522 | 21751572 |
| chr21 | 22301691 | 22301741 |
| chr21 | 22626598 | 22626648 |
| chr21 | 25249060 | 25249110 |
| chr21 | 25547992 | 25548042 |
| chr21 | 26208640 | 26208690 |
| chr21 | 26554072 | 26554122 |
| chr21 | 27762911 | 27762961 |
| chr21 | 29188453 | 29188503 |
| chr21 | 29370672 | 29370722 |
| chr21 | 31662159 | 31662209 |
| chr21 | 31788138 | 31788188 |
| chr21 | 32178136 | 32178186 |
| chr21 | 34703931 | 34703981 |
| chr21 | 37179223 | 37179273 |
| chr21 | 37597268 | 37597318 |
| chr21 | 39002331 | 39002381 |
| chr21 | 41170710 | 41170760 |
| chr21 | 42526742 | 42526792 |
| chr21 | 42940476 | 42940526 |
| chr21 | 43098080 | 43098130 |
| chr21 | 43549330 | 43549380 |
| chr21 | 43555204 | 43555254 |
| chr11 | 878649 | 878699 |
| chr21 | 43761456 | 43761506 |
| chr21 | 44028131 | 44028181 |
| chr11 | 950819 | 950869 |
| chr21 | 45065655 | 45065705 |
| chr21 | 45284617 | 45284667 |
| chr11 | 1044225 | 1044275 |
| chr21 | 46221250 | 46221300 |
| chr21 | 46703510 | 46703560 |
| chr11 | 1351881 | 1351931 |
| chr22 | 19140078 | 19140128 |
| chr22 | 20581099 | 20581149 |
| chr22 | 21687007 | 21687057 |
| chr22 | 22238340 | 22238390 |
| chr22 | 22766221 | 22766271 |
| chr11 | 1594504 | 1594554 |
| chr11 | 1594345 | 1594395 |
| chr22 | 23538245 | 23538295 |
| chr22 | 23539287 | 23539337 |
| chr22 | 23785817 | 23785867 |
| chr22 | 32146304 | 32146354 |
| chr22 | 32607869 | 32607919 |
| chr22 | 32646888 | 32646938 |
| chr22 | 33442151 | 33442201 |
| chr22 | 33724834 | 33724884 |
| chr22 | 35078060 | 35078110 |
| chr22 | 35528365 | 35528415 |
| chr22 | 35694072 | 35694122 |
| chr22 | 36169523 | 36169573 |
| chr22 | 36828976 | 36829026 |
| chr11 | 2314129 | 2314179 |
| chr11 | 2338162 | 2338212 |
| chr11 | 2338614 | 2338664 |
| chr22 | 37972796 | 37972846 |
| chr22 | 38370087 | 38370137 |
| chr22 | 39032518 | 39032568 |
| chr22 | 39546152 | 39546202 |
| chr22 | 40042161 | 40042211 |
| chr11 | 2592794 | 2592844 |
| chr22 | 41232035 | 41232085 |
| chr22 | 42646366 | 42646416 |
| chr22 | 43287757 | 43287807 |
| chr22 | 43513340 | 43513390 |
| chr22 | 44784989 | 44785039 |
| chr22 | 44900119 | 44900169 |
| chr22 | 44932858 | 44932908 |
| chr22 | 45049461 | 45049511 |
| chr22 | 45746292 | 45746342 |
| chr22 | 46078808 | 46078858 |
| chr22 | 46080758 | 46080808 |
| chr22 | 46227780 | 46227830 |
| chr22 | 46228591 | 46228641 |
| chr22 | 46282690 | 46282740 |
| chr22 | 46817268 | 46817318 |
| chr22 | 47166765 | 47166815 |
| chr22 | 47287788 | 47287838 |
| chr22 | 47305008 | 47305058 |
| chr22 | 47403855 | 47403905 |
| chr22 | 47417932 | 47417982 |
| chr22 | 47519185 | 47519235 |
| chr22 | 47658403 | 47658453 |
| chr22 | 47790413 | 47790463 |
| chr22 | 47792067 | 47792117 |
| chr22 | 48097979 | 48098029 |
| chr22 | 48808960 | 48809010 |
| chrX | 3166567 | 3166617 |
| chrX | 3218639 | 3218689 |
| chrX | 3693682 | 3693732 |
| chrX | 5040638 | 5040688 |
| chrX | 6069324 | 6069374 |
| chrX | 6070646 | 6070696 |
| chrX | 6275503 | 6275553 |
| chrX | 8764124 | 8764174 |
| chrX | 9209951 | 9210001 |
| chrX | 9385793 | 9385843 |
| chr11 | 3918268 | 3918318 |
| chrX | 9963176 | 9963226 |
| chrX | 10511435 | 10511485 |
| chrX | 11334801 | 11334851 |
| chrX | 12186608 | 12186658 |
| chrX | 15137566 | 15137616 |
| chrX | 15726887 | 15726937 |
| chrX | 15828443 | 15828493 |
| chrX | 18041565 | 18041615 |
| chrX | 27110709 | 27110759 |
| chrX | 28047694 | 28047744 |
| chrX | 30333009 | 30333059 |
| chr11 | 6298286 | 6298336 |
| chrX | 33923431 | 33923481 |
| chrX | 34424980 | 34425030 |
| chrX | 38745528 | 38745578 |
| chrX | 39592389 | 39592439 |
| chrX | 40347585 | 40347635 |
| chr11 | 6732140 | 6732190 |
| chr11 | 6731923 | 6731973 |
| chrX | 43354950 | 43355000 |
| chrX | 43564155 | 43564205 |
| chrX | 49249955 | 49250005 |
| chrX | 49250339 | 49250389 |
| chrX | 53120497 | 53120547 |
| chrX | 56378890 | 56378940 |
| chrX | 62485450 | 62485500 |
| chrX | 66991661 | 66991711 |
| chrX | 70339313 | 70339363 |
| chr11 | 8838905 | 8838955 |
| chrX | 88566071 | 88566121 |
| chrX | 92964870 | 92964920 |
| chrX | 93904203 | 93904253 |
| chrX | 94820214 | 94820264 |
| chrX | 96345770 | 96345820 |
| chrX | 96474223 | 96474273 |
| chrX | 101121222 | 101121272 |
| chrX | 104123657 | 104123707 |
| chrX | 111518906 | 111518956 |
| chrX | 112216226 | 112216276 |
| chrX | 113679021 | 113679071 |
| chrX | 115685448 | 115685498 |
| chrX | 117279575 | 117279625 |
| chrX | 117534168 | 117534218 |
| chrX | 117659874 | 117659924 |
| chrX | 118283500 | 118283550 |
| chrX | 120903830 | 120903880 |
| chrX | 126492679 | 126492729 |
| chrX | 127992987 | 127993037 |
| chr11 | 11221798 | 11221848 |
| chrX | 131904454 | 131904504 |
| chrX | 132306486 | 132306536 |
| chrX | 132365114 | 132365164 |
| chr11 | 11571545 | 11571595 |
| chrX | 134676196 | 134676246 |
| chrX | 136577930 | 136577980 |
| chrX | 136679133 | 136679183 |
| chrX | 138091288 | 138091338 |
| chrX | 144073935 | 144073985 |
| chrX | 144677977 | 144678027 |
| chrX | 149399945 | 149399995 |
| chrX | 149463560 | 149463610 |
| chrX | 150270932 | 150270982 |
| chrX | 150652116 | 150652166 |
| chrX | 150651952 | 150652002 |
| chr11 | 13769871 | 13769921 |
| chrY | 3541769 | 3541819 |
| chrY | 3688002 | 3688052 |
| chrY | 9052531 | 9052581 |
| chrY | 9419459 | 9419509 |
| chrY | 16330374 | 16330424 |
| chrY | 17215880 | 17215930 |
| chr2 | 284574 | 284624 |
| chr2 | 385960 | 386010 |
| chr11 | 15298355 | 15298405 |
| chr2 | 1070590 | 1070640 |
| chr2 | 1146344 | 1146394 |
| chr11 | 15498379 | 15498429 |
| chr2 | 1521082 | 1521132 |
| chr2 | 1764358 | 1764408 |
| chr2 | 2579496 | 2579546 |
| chr2 | 2826705 | 2826755 |
| chr2 | 3148523 | 3148573 |
| chr2 | 3182823 | 3182873 |
| chr2 | 3245915 | 3245965 |
| chr2 | 3346018 | 3346068 |
| chr2 | 4023543 | 4023593 |
| chr2 | 5587007 | 5587057 |
| chr11 | 17084650 | 17084700 |
| chr2 | 9840685 | 9840735 |
| chr2 | 14754314 | 14754364 |
| chr2 | 15748891 | 15748941 |
| chr2 | 18031329 | 18031379 |
| chr2 | 21785684 | 21785734 |
| chr2 | 24564515 | 24564565 |
| chr2 | 28114831 | 28114881 |
| chr11 | 18716030 | 18716080 |
| chr2 | 29802542 | 29802592 |
| chr2 | 30350587 | 30350637 |
| chr2 | 30522311 | 30522361 |
| chr2 | 31095126 | 31095176 |
| chr2 | 32903249 | 32903299 |
| chr2 | 36348079 | 36348129 |
| chr2 | 38102124 | 38102174 |
| chr10 | 636818 | 636868 |
| chr2 | 39246423 | 39246473 |
| chr2 | 42217298 | 42217348 |
| chr2 | 43183865 | 43183915 |
| chr2 | 47317603 | 47317653 |
| chr2 | 48767006 | 48767056 |
| chr2 | 48804122 | 48804172 |
| chr2 | 51905688 | 51905738 |
| chr11 | 22125717 | 22125767 |
| chr2 | 54218673 | 54218723 |
| chr11 | 22421686 | 22421736 |
| chr2 | 55902936 | 55902986 |
| chr2 | 55907293 | 55907343 |
| chr2 | 56326920 | 56326970 |
| chr2 | 58927586 | 58927636 |
| chr2 | 59654211 | 59654261 |
| chr2 | 62466065 | 62466115 |
| chr2 | 64804357 | 64804407 |
| chr2 | 64990865 | 64990915 |
| chr2 | 69495081 | 69495131 |
| chr2 | 69834922 | 69834972 |
| chr10 | 11805059 | 11805109 |
| chr2 | 71293417 | 71293467 |
| chr2 | 72415843 | 72415893 |
| chr2 | 73580220 | 73580270 |
| chr2 | 76966495 | 76966545 |
| chr2 | 80164377 | 80164427 |
| chr2 | 86755280 | 86755330 |
| chr2 | 87312463 | 87312513 |
| chr2 | 87399101 | 87399151 |
| chr11 | 28484301 | 28484351 |
| chr2 | 94867598 | 94867648 |
| chr11 | 28856682 | 28856732 |
| chr2 | 100216551 | 100216601 |
| chr2 | 100216389 | 100216439 |
| chr2 | 100891723 | 100891773 |
| chr2 | 101033894 | 101033944 |
| chr2 | 101665805 | 101665855 |
| chr2 | 104107871 | 104107921 |
| chr2 | 104406101 | 104406151 |
| chr2 | 105528900 | 105528950 |
| chr2 | 105696530 | 105696580 |
| chr2 | 105788667 | 105788717 |
| chr2 | 109269484 | 109269534 |
| chr2 | 109484189 | 109484239 |
| chr2 | 112131838 | 112131888 |
| chr2 | 112539179 | 112539229 |
| chr2 | 112620962 | 112621012 |
| chr11 | 32769707 | 32769757 |
| chr2 | 122371275 | 122371325 |
| chr2 | 122803434 | 122803484 |
| chr2 | 128909857 | 128909907 |
| chr2 | 129058858 | 129058908 |
| chr2 | 129366289 | 129366339 |
| chr2 | 130316228 | 130316278 |
| chr2 | 135600950 | 135601000 |
| chr2 | 140885458 | 140885508 |
| chr2 | 150406171 | 150406221 |
| chr2 | 150618504 | 150618554 |
| chr2 | 152070570 | 152070620 |
| chr2 | 153448657 | 153448707 |
| chr2 | 158561622 | 158561672 |
| chr2 | 158893522 | 158893572 |
| chr2 | 159728955 | 159729005 |
| chr2 | 159970429 | 159970479 |
| chr2 | 164354863 | 164354913 |
| chr2 | 168567188 | 168567238 |
| chr2 | 170593329 | 170593379 |
| chr2 | 170780631 | 170780681 |
| chr2 | 170902111 | 170902161 |
| chr11 | 36185477 | 36185527 |
| chr2 | 174875027 | 174875077 |
| chr2 | 177276011 | 177276061 |
| chr2 | 181446697 | 181446747 |
| chr2 | 185274141 | 185274191 |
| chr2 | 186997185 | 186997235 |
| chr2 | 188311216 | 188311266 |
| chr2 | 190360894 | 190360944 |
| chr2 | 190464390 | 190464440 |
| chr11 | 39073247 | 39073297 |
| chr2 | 196412535 | 196412585 |
| chr2 | 198419777 | 198419827 |
| chr2 | 201863185 | 201863235 |
| chr2 | 201902010 | 201902060 |
| chr11 | 40147681 | 40147731 |
| chr2 | 203318852 | 203318902 |
| chr2 | 203573852 | 203573902 |
| chr2 | 204114085 | 204114135 |
| chr2 | 204818883 | 204818933 |
| chr2 | 208729015 | 208729065 |
| chr2 | 209445754 | 209445804 |
| chr2 | 210058441 | 210058491 |
| chr2 | 213618425 | 213618475 |
| chr2 | 215419112 | 215419162 |
| chr2 | 215921092 | 215921142 |
| chr2 | 216626792 | 216626842 |
| chr2 | 217592477 | 217592527 |
| chr11 | 44047219 | 44047269 |
| chr2 | 225515562 | 225515612 |
| chr2 | 226175822 | 226175872 |
| chr2 | 232029091 | 232029141 |
| chr2 | 233997498 | 233997548 |
| chr2 | 234426729 | 234426779 |
| chr2 | 235714871 | 235714921 |
| chr2 | 236256659 | 236256709 |
| chr2 | 236948295 | 236948345 |
| chr2 | 236948579 | 236948629 |
| chr2 | 237300901 | 237300951 |
| chr2 | 238236795 | 238236845 |
| chr11 | 45717457 | 45717507 |
| chr2 | 238835953 | 238836003 |
| chr2 | 238858390 | 238858440 |
| chr11 | 45798215 | 45798265 |
| chr2 | 239749814 | 239749864 |
| chr2 | 239750109 | 239750159 |
| chr2 | 239996012 | 239996062 |
| chr2 | 241479402 | 241479452 |
| chr2 | 241504874 | 241504924 |
| chr2 | 241587406 | 241587456 |
| chr2 | 241633758 | 241633808 |
| chr2 | 243080706 | 243080756 |
| chr3 | 2811950 | 2812000 |
| chr3 | 3022512 | 3022562 |
| chr3 | 4580965 | 4581015 |
| chr3 | 5594478 | 5594528 |
| chr3 | 5904818 | 5904868 |
| chr3 | 6243256 | 6243306 |
| chr3 | 7184754 | 7184804 |
| chr3 | 9258999 | 9259049 |
| chr3 | 10747574 | 10747624 |
| chr3 | 11606318 | 11606368 |
| chr3 | 11757313 | 11757363 |
| chr3 | 11791248 | 11791298 |
| chr3 | 13572806 | 13572856 |
| chr3 | 14935771 | 14935821 |
| chr3 | 17131930 | 17131980 |
| chr3 | 23152294 | 23152344 |
| chr3 | 23902000 | 23902050 |
| chr3 | 25873074 | 25873124 |
| chr3 | 27754063 | 27754113 |
| chr3 | 29132794 | 29132844 |
| chr3 | 30028531 | 30028581 |
| chr3 | 31851455 | 31851505 |
| chr3 | 32811215 | 32811265 |
| chr3 | 32811877 | 32811927 |
| chr3 | 35375834 | 35375884 |
| chr3 | 37186933 | 37186983 |
| chr3 | 38018676 | 38018726 |
| chr3 | 39256408 | 39256458 |
| chr11 | 48058072 | 48058122 |
| chr3 | 44496232 | 44496282 |
| chr3 | 45433145 | 45433195 |
| chr3 | 45589721 | 45589771 |
| chr3 | 45658418 | 45658468 |
| chr3 | 47234755 | 47234805 |
| chr3 | 52125602 | 52125652 |
| chr3 | 52983102 | 52983152 |
| chr3 | 57645414 | 57645464 |
| chr3 | 58095856 | 58095906 |
| chr3 | 63618129 | 63618179 |
| chr3 | 69035014 | 69035064 |
| chr11 | 58010587 | 58010637 |
| chr3 | 76894807 | 76894857 |
| chr3 | 78914744 | 78914794 |
| chr3 | 79990168 | 79990218 |
| chr3 | 81149478 | 81149528 |
| chr3 | 86019685 | 86019735 |
| chr3 | 86655220 | 86655270 |
| chr3 | 88446965 | 88447015 |
| chr3 | 89351303 | 89351353 |
| chr3 | 95573782 | 95573832 |
| chr3 | 96296775 | 96296825 |
| chr3 | 97063933 | 97063983 |
| chr3 | 101444828 | 101444878 |
| chr3 | 102658141 | 102658191 |
| chr3 | 103556591 | 103556641 |
| chr3 | 104602332 | 104602382 |
| chr3 | 105270010 | 105270060 |
| chr3 | 108879852 | 108879902 |
| chr3 | 110388685 | 110388735 |
| chr3 | 110456527 | 110456577 |
| chr3 | 110623912 | 110623962 |
| chr3 | 114991901 | 114991951 |
| chr3 | 116814508 | 116814558 |
| chr3 | 117717428 | 117717478 |
| chr3 | 119211147 | 119211197 |
| chr3 | 119212344 | 119212394 |
| chr3 | 120426357 | 120426407 |
| chr3 | 121452504 | 121452554 |
| chr10 | 12768049 | 12768099 |
| chr3 | 129743515 | 129743565 |
| chr3 | 130776902 | 130776952 |
| chr3 | 133828739 | 133828789 |
| chr3 | 135433419 | 135433469 |
| chr3 | 139058402 | 139058452 |
| chr3 | 142349181 | 142349231 |
| chr3 | 143039830 | 143039880 |
| chr3 | 144547246 | 144547296 |
| chr3 | 145789345 | 145789395 |
| chr3 | 145790891 | 145790941 |
| chr3 | 151624751 | 151624801 |
| chr3 | 151733942 | 151733992 |
| chr3 | 152324205 | 152324255 |
| chr3 | 152657110 | 152657160 |
| chr3 | 154879078 | 154879128 |
| chr3 | 156099843 | 156099893 |
| chr3 | 158351515 | 158351565 |
| chr3 | 159776801 | 159776851 |
| chr3 | 161184707 | 161184757 |
| chr3 | 163162516 | 163162566 |
| chr3 | 169785059 | 169785109 |
| chr3 | 172967705 | 172967755 |
| chr3 | 175400616 | 175400666 |
| chr3 | 175561778 | 175561828 |
| chr3 | 176021815 | 176021865 |
| chr3 | 176289003 | 176289053 |
| chr3 | 178245259 | 178245309 |
| chr3 | 180143027 | 180143077 |
| chr3 | 180148641 | 180148691 |
| chr3 | 182488050 | 182488100 |
| chr3 | 184502437 | 184502487 |
| chr3 | 184728547 | 184728597 |
| chr3 | 186107482 | 186107532 |
| chr3 | 187066477 | 187066527 |
| chr3 | 187870294 | 187870344 |
| chr3 | 194414717 | 194414767 |
| chr3 | 194875262 | 194875312 |
| chr11 | 63471638 | 63471688 |
| chr3 | 198334327 | 198334377 |
| chr4 | 697614 | 697664 |
| chr4 | 909006 | 909056 |
| chr4 | 1048419 | 1048469 |
| chr4 | 1609924 | 1609974 |
| chr4 | 3680591 | 3680641 |
| chr4 | 4652175 | 4652225 |
| chr4 | 4651920 | 4651970 |
| chr4 | 5471508 | 5471558 |
| chr4 | 6621795 | 6621845 |
| chr4 | 6691082 | 6691132 |
| chr4 | 6692774 | 6692824 |
| chr4 | 7003045 | 7003095 |
| chr4 | 7224898 | 7224948 |
| chr4 | 7236218 | 7236268 |
| chr4 | 8323576 | 8323626 |
| chr4 | 8448890 | 8448940 |
| chr4 | 8838583 | 8838633 |
| chr4 | 10581814 | 10581864 |
| chr4 | 13422068 | 13422118 |
| chr4 | 13607805 | 13607855 |
| chr4 | 15161058 | 15161108 |
| chr4 | 18652069 | 18652119 |
| chr4 | 19174707 | 19174757 |
| chr4 | 19715211 | 19715261 |
| chr4 | 25658397 | 25658447 |
| chr4 | 26863646 | 26863696 |
| chr4 | 28966347 | 28966397 |
| chr4 | 31399089 | 31399139 |
| chr11 | 64700890 | 64700940 |
| chr4 | 37033869 | 37033919 |
| chr4 | 38628292 | 38628342 |
| chr4 | 38826368 | 38826418 |
| chr4 | 39294338 | 39294388 |
| chr4 | 39486197 | 39486247 |
| chr4 | 42992684 | 42992734 |
| chr4 | 45395172 | 45395222 |
| chr4 | 45519823 | 45519873 |
| chr4 | 47493774 | 47493824 |
| chr4 | 53871027 | 53871077 |
| chr4 | 55268569 | 55268619 |
| chr4 | 55398398 | 55398448 |
| chr4 | 56995897 | 56995947 |
| chr4 | 57138589 | 57138639 |
| chr4 | 57552554 | 57552604 |
| chr11 | 65218662 | 65218712 |
| chr11 | 65286404 | 65286454 |
| chr4 | 63361730 | 63361780 |
| chr4 | 63395615 | 63395665 |
| chr4 | 63602380 | 63602430 |
| chr4 | 63915993 | 63916043 |
| chr4 | 65749782 | 65749832 |
| chr4 | 67171406 | 67171456 |
| chr4 | 67429475 | 67429525 |
| chr4 | 67552484 | 67552534 |
| chr4 | 67864885 | 67864935 |
| chr4 | 69163433 | 69163483 |
| chr4 | 76836604 | 76836654 |
| chr4 | 84167857 | 84167907 |
| chr4 | 87775577 | 87775627 |
| chr4 | 94193263 | 94193313 |
| chr4 | 96900427 | 96900477 |
| chr4 | 97351441 | 97351491 |
| chr4 | 100340653 | 100340703 |
| chr4 | 101492283 | 101492333 |
| chr4 | 102701865 | 102701915 |
| chr4 | 103537099 | 103537149 |
| chr4 | 110010863 | 110010913 |
| chr4 | 110992770 | 110992820 |
| chr4 | 112143589 | 112143639 |
| chr4 | 112504259 | 112504309 |
| chr11 | 66194568 | 66194618 |
| chr4 | 114860281 | 114860331 |
| chr4 | 115894994 | 115895044 |
| chr4 | 117710246 | 117710296 |
| chr11 | 66312473 | 66312523 |
| chr4 | 124460007 | 124460057 |
| chr11 | 66485955 | 66486005 |
| chr4 | 130006317 | 130006367 |
| chr4 | 130046926 | 130046976 |
| chr4 | 130092495 | 130092545 |
| chr4 | 131684430 | 131684480 |
| chr4 | 131804841 | 131804891 |
| chr4 | 135350975 | 135351025 |
| chr11 | 66644406 | 66644456 |
| chr4 | 140386792 | 140386842 |
| chr4 | 140395544 | 140395594 |
| chr4 | 140426584 | 140426634 |
| chr4 | 144828129 | 144828179 |
| chr4 | 147682827 | 147682877 |
| chr4 | 148125040 | 148125090 |
| chr4 | 149194409 | 149194459 |
| chr4 | 154068479 | 154068529 |
| chr4 | 154737589 | 154737639 |
| chr4 | 155316431 | 155316481 |
| chr4 | 155316301 | 155316351 |
| chr4 | 157773495 | 157773545 |
| chr4 | 162201067 | 162201117 |
| chr4 | 167546643 | 167546693 |
| chr4 | 170865875 | 170865925 |
| chr4 | 174185874 | 174185924 |
| chr4 | 176413262 | 176413312 |
| chr4 | 177200435 | 177200485 |
| chr4 | 177549385 | 177549435 |
| chr4 | 178027984 | 178028034 |
| chr4 | 180364150 | 180364200 |
| chr4 | 180860950 | 180861000 |
| chr4 | 181278905 | 181278955 |
| chr4 | 183424077 | 183424127 |
| chr4 | 183705996 | 183706046 |
| chr4 | 184523044 | 184523094 |
| chr4 | 184523285 | 184523335 |
| chr4 | 184523433 | 184523483 |
| chr4 | 186989358 | 186989408 |
| chr11 | 68203518 | 68203568 |
| chr4 | 188156277 | 188156327 |
| chr4 | 188156435 | 188156485 |
| chr4 | 188919134 | 188919184 |
| chr4 | 189190536 | 189190586 |
| chr4 | 189298899 | 189298949 |
| chr4 | 189675471 | 189675521 |
| chr4 | 191203857 | 191203907 |
| chr11 | 68557276 | 68557326 |
| chr5 | 777926 | 777976 |
| chr5 | 862246 | 862296 |
| chr5 | 1147906 | 1147956 |
| chr5 | 1433465 | 1433515 |
| chr5 | 1712924 | 1712974 |
| chr5 | 1847279 | 1847329 |
| chr5 | 1880981 | 1881031 |
| chr5 | 3083945 | 3083995 |
| chr5 | 3125550 | 3125600 |
| chr5 | 3152339 | 3152389 |
| chr5 | 6075254 | 6075304 |
| chr5 | 6075012 | 6075062 |
| chr5 | 6575603 | 6575653 |
| chr5 | 6848267 | 6848317 |
| chr5 | 6908181 | 6908231 |
| chr5 | 9470759 | 9470809 |
| chr5 | 10345753 | 10345803 |
| chr5 | 10361782 | 10361832 |
| chr5 | 10593174 | 10593224 |
| chr5 | 10665516 | 10665566 |
| chr5 | 10912750 | 10912800 |
| chr5 | 12034007 | 12034057 |
| chr5 | 12239006 | 12239056 |
| chr5 | 17194276 | 17194326 |
| chr5 | 18465138 | 18465188 |
| chr5 | 18746772 | 18746822 |
| chr5 | 18776817 | 18776867 |
| chr5 | 20739594 | 20739644 |
| chr5 | 20815949 | 20815999 |
| chr5 | 20816003 | 20816053 |
| chr5 | 22842417 | 22842467 |
| chr5 | 23252956 | 23253006 |
| chr5 | 24018565 | 24018615 |
| chr5 | 26248755 | 26248805 |
| chr5 | 28233868 | 28233918 |
| chr5 | 28360930 | 28360980 |
| chr5 | 29346919 | 29346969 |
| chr5 | 29485164 | 29485214 |
| chr5 | 29973586 | 29973636 |
| chr5 | 31940504 | 31940554 |
| chr5 | 39144778 | 39144828 |
| chr5 | 43235815 | 43235865 |
| chr5 | 50601352 | 50601402 |
| chr5 | 50884057 | 50884107 |
| chr5 | 51447251 | 51447301 |
| chr5 | 52933871 | 52933921 |
| chr5 | 56102439 | 56102489 |
| chr5 | 57211149 | 57211199 |
| chr5 | 59494808 | 59494858 |
| chr11 | 70437659 | 70437709 |
| chr11 | 70548001 | 70548051 |
| chr5 | 72205071 | 72205121 |
| chr5 | 77572039 | 77572089 |
| chr5 | 78022066 | 78022116 |
| chr5 | 78022206 | 78022256 |
| chr5 | 78139794 | 78139844 |
| chr5 | 78464455 | 78464505 |
| chr5 | 79733733 | 79733783 |
| chr5 | 86014941 | 86014991 |
| chr5 | 86223367 | 86223417 |
| chr5 | 92884588 | 92884638 |
| chr5 | 95417751 | 95417801 |
| chr5 | 96484259 | 96484309 |
| chr11 | 71379655 | 71379705 |
| chr5 | 97303752 | 97303802 |
| chr5 | 99282775 | 99282825 |
| chr5 | 102082978 | 102083028 |
| chr5 | 103393283 | 103393333 |
| chr5 | 108101089 | 108101139 |
| chr5 | 108808701 | 108808751 |
| chr5 | 110886759 | 110886809 |
| chr5 | 113696881 | 113696931 |
| chr5 | 115442130 | 115442180 |
| chr5 | 116047655 | 116047705 |
| chr5 | 124793979 | 124794029 |
| chr5 | 125620982 | 125621032 |
| chr5 | 130011690 | 130011740 |
| chr5 | 130705553 | 130705603 |
| chr5 | 131603059 | 131603109 |
| chr5 | 133427436 | 133427486 |
| chr5 | 134816979 | 134817029 |
| chr11 | 72795695 | 72795745 |
| chr5 | 136708348 | 136708398 |
| chr5 | 136782054 | 136782104 |
| chr5 | 137994043 | 137994093 |
| chr10 | 14594752 | 14594802 |
| chr5 | 141480312 | 141480362 |
| chr5 | 143748499 | 143748549 |
| chr5 | 145107083 | 145107133 |
| chr5 | 145619040 | 145619090 |
| chr5 | 145927213 | 145927263 |
| chr5 | 146607337 | 146607387 |
| chr11 | 73994890 | 73994940 |
| chr5 | 151170991 | 151171041 |
| chr5 | 152241936 | 152241986 |
| chr5 | 162763781 | 162763831 |
| chr5 | 164347721 | 164347771 |
| chr5 | 164922120 | 164922170 |
| chr5 | 165418224 | 165418274 |
| chr5 | 165683010 | 165683060 |
| chr5 | 171249875 | 171249925 |
| chr5 | 171733637 | 171733687 |
| chr5 | 173231340 | 173231390 |
| chr5 | 176398135 | 176398185 |
| chr5 | 177361949 | 177361999 |
| chr5 | 177988629 | 177988679 |
| chr5 | 178852453 | 178852503 |
| chr5 | 179009843 | 179009893 |
| chr11 | 76481368 | 76481418 |
| chr5 | 180370282 | 180370332 |
| chr5 | 180600261 | 180600311 |
| chr5 | 180641034 | 180641084 |
| chr6 | 2116431 | 2116481 |
| chr6 | 3441172 | 3441222 |
| chr6 | 3911509 | 3911559 |
| chr6 | 4036412 | 4036462 |
| chr11 | 77155761 | 77155811 |
| chr6 | 4756240 | 4756290 |
| chr6 | 7267792 | 7267842 |
| chr6 | 7649893 | 7649943 |
| chr6 | 7846279 | 7846329 |
| chr6 | 7879782 | 7879832 |
| chr6 | 9071316 | 9071366 |
| chr6 | 9290298 | 9290348 |
| chr11 | 77602056 | 77602106 |
| chr6 | 10808596 | 10808646 |
| chr6 | 11197828 | 11197878 |
| chr6 | 13320864 | 13320914 |
| chr6 | 13609268 | 13609318 |
| chr6 | 13885006 | 13885056 |
| chr6 | 14176842 | 14176892 |
| chr6 | 14494024 | 14494074 |
| chr6 | 15126278 | 15126328 |
| chr6 | 15638844 | 15638894 |
| chr6 | 16799779 | 16799829 |
| chr6 | 17509126 | 17509176 |
| chr11 | 80295986 | 80296036 |
| chr6 | 23212789 | 23212839 |
| chr11 | 81202525 | 81202575 |
| chr11 | 81375698 | 81375748 |
| chr6 | 27540814 | 27540864 |
| chr6 | 27880006 | 27880056 |
| chr6 | 28258790 | 28258840 |
| chr6 | 29141233 | 29141283 |
| chr6 | 32636170 | 32636220 |
| chr6 | 33244973 | 33245023 |
| chr11 | 83858300 | 83858350 |
| chr6 | 37828214 | 37828264 |
| chr6 | 37856401 | 37856451 |
| chr6 | 38410709 | 38410759 |
| chr6 | 39084428 | 39084478 |
| chr6 | 39843570 | 39843620 |
| chr6 | 42481945 | 42481995 |
| chr6 | 45270899 | 45270949 |
| chr6 | 47389001 | 47389051 |
| chr6 | 56219731 | 56219781 |
| chr6 | 57315527 | 57315577 |
| chr6 | 66751990 | 66752040 |
| chr6 | 69185129 | 69185179 |
| chr6 | 70163184 | 70163234 |
| chr6 | 79183274 | 79183324 |
| chr6 | 80443774 | 80443824 |
| chr6 | 83708015 | 83708065 |
| chr11 | 92627531 | 92627581 |
| chr6 | 94530234 | 94530284 |
| chr6 | 97833505 | 97833555 |
| chr6 | 99463169 | 99463219 |
| chr6 | 107220430 | 107220480 |
| chr6 | 107490894 | 107490944 |
| chr6 | 109873160 | 109873210 |
| chr6 | 111893885 | 111893935 |
| chr6 | 114960965 | 114961015 |
| chr6 | 119293784 | 119293834 |
| chr6 | 122118195 | 122118245 |
| chr6 | 124479787 | 124479837 |
| chr6 | 124725475 | 124725525 |
| chr6 | 125187748 | 125187798 |
| chr11 | 96206549 | 96206599 |
| chr6 | 131255497 | 131255547 |
| chr6 | 132642644 | 132642694 |
| chr6 | 135747843 | 135747893 |
| chr6 | 136618302 | 136618352 |
| chr6 | 139492745 | 139492795 |
| chr6 | 139635285 | 139635335 |
| chr6 | 139948162 | 139948212 |
| chr6 | 141062625 | 141062675 |
| chr6 | 145596648 | 145596698 |
| chr6 | 147959356 | 147959406 |
| chr6 | 151842009 | 151842059 |
| chr6 | 154987666 | 154987716 |
| chr6 | 155502927 | 155502977 |
| chr6 | 156280242 | 156280292 |
| chr6 | 158194965 | 158195015 |
| chr6 | 158355412 | 158355462 |
| chr6 | 159076567 | 159076617 |
| chr6 | 160049792 | 160049842 |
| chr11 | 101862778 | 101862828 |
| chr6 | 160601776 | 160601826 |
| chr6 | 161256909 | 161256959 |
| chr6 | 161257307 | 161257357 |
| chr6 | 161257548 | 161257598 |
| chr11 | 102056840 | 102056890 |
| chr6 | 161931711 | 161931761 |
| chr6 | 164255458 | 164255508 |
| chr11 | 102935242 | 102935292 |
| chr11 | 103269781 | 103269831 |
| chr6 | 166581842 | 166581892 |
| chr6 | 166845192 | 166845242 |
| chr6 | 167839019 | 167839069 |
| chr11 | 104683558 | 104683608 |
| chr6 | 167937651 | 167937701 |
| chr6 | 168145501 | 168145551 |
| chr6 | 168145801 | 168145851 |
| chr6 | 168352323 | 168352373 |
| chr6 | 168352795 | 168352845 |
| chr6 | 168441729 | 168441779 |
| chr6 | 168897615 | 168897665 |
| chr6 | 169000339 | 169000389 |
| chr6 | 169000587 | 169000637 |
| chr6 | 169000863 | 169000913 |
| chr6 | 169784417 | 169784467 |
| chr6 | 170249349 | 170249399 |
| chr11 | 106984380 | 106984430 |
| chr6 | 170650606 | 170650656 |
| chr7 | 1479814 | 1479864 |
| chr7 | 1638082 | 1638132 |
| chr7 | 1638917 | 1638967 |
| chr7 | 2523973 | 2524023 |
| chr7 | 2524713 | 2524763 |
| chr7 | 3884934 | 3884984 |
| chr7 | 4112694 | 4112744 |
| chr7 | 4433335 | 4433385 |
| chr7 | 4732835 | 4732885 |
| chr7 | 5967977 | 5968027 |
| chr7 | 11004196 | 11004246 |
| chr7 | 13637005 | 13637055 |
| chr7 | 13862019 | 13862069 |
| chr7 | 14028302 | 14028352 |
| chr7 | 15356821 | 15356871 |
| chr7 | 16949412 | 16949462 |
| chr7 | 23411080 | 23411130 |
| chr7 | 23410802 | 23410852 |
| chr7 | 23669707 | 23669757 |
| chr7 | 23707879 | 23707929 |
| chr7 | 25888826 | 25888876 |
| chr7 | 27435310 | 27435360 |
| chr7 | 29451979 | 29452029 |
| chr7 | 32525446 | 32525496 |
| chr7 | 33213725 | 33213775 |
| chr7 | 43106098 | 43106148 |
| chr7 | 43332609 | 43332659 |
| chr7 | 43738532 | 43738582 |
| chr11 | 113019929 | 113019979 |
| chr7 | 44896111 | 44896161 |
| chr7 | 44900805 | 44900855 |
| chr7 | 45032493 | 45032543 |
| chr7 | 45266056 | 45266106 |
| chr11 | 113337980 | 113338030 |
| chr7 | 47398572 | 47398622 |
| chr7 | 47436861 | 47436911 |
| chr7 | 48022704 | 48022754 |
| chr7 | 49011312 | 49011362 |
| chr7 | 50677319 | 50677369 |
| chr7 | 50834675 | 50834725 |
| chr7 | 70887096 | 70887146 |
| chr7 | 72357940 | 72357990 |
| chr7 | 74824923 | 74824973 |
| chr7 | 79780658 | 79780708 |
| chr7 | 84044841 | 84044891 |
| chr7 | 99790839 | 99790889 |
| chr7 | 100395349 | 100395399 |
| chr7 | 103764666 | 103764716 |
| chr7 | 104632041 | 104632091 |
| chr7 | 106930896 | 106930946 |
| chr7 | 117656162 | 117656212 |
| chr7 | 124636855 | 124636905 |
| chr7 | 128018715 | 128018765 |
| chr7 | 128727639 | 128727689 |
| chr7 | 135314001 | 135314051 |
| chr7 | 137714541 | 137714591 |
| chr7 | 138997089 | 138997139 |
| chr7 | 140850214 | 140850264 |
| chr10 | 17612669 | 17612719 |
| chr7 | 143645153 | 143645203 |
| chr7 | 144533638 | 144533688 |
| chr7 | 144533581 | 144533631 |
| chr7 | 146183546 | 146183596 |
| chr11 | 118845396 | 118845446 |
| chr11 | 118849783 | 118849833 |
| chr7 | 147876480 | 147876530 |
| chr7 | 149612898 | 149612948 |
| chr7 | 150543396 | 150543446 |
| chr11 | 119287375 | 119287425 |
| chr7 | 150807307 | 150807357 |
| chr7 | 152672227 | 152672277 |
| chr7 | 152887138 | 152887188 |
| chr7 | 153017792 | 153017842 |
| chr7 | 154231213 | 154231263 |
| chr7 | 154791724 | 154791774 |
| chr11 | 119980741 | 119980791 |
| chr7 | 157481674 | 157481724 |
| chr7 | 157520136 | 157520186 |
| chr7 | 158043621 | 158043671 |
| chr7 | 158044757 | 158044807 |
| chr7 | 158249517 | 158249567 |
| chr7 | 158249299 | 158249349 |
| chr10 | 17966490 | 17966540 |
| chr8 | 1583404 | 1583454 |
| chr8 | 1719367 | 1719417 |
| chr8 | 1719962 | 1720012 |
| chr8 | 1962273 | 1962323 |
| chr8 | 1979526 | 1979576 |
| chr10 | 18213400 | 18213450 |
| chr8 | 2615962 | 2616012 |
| chr8 | 2767365 | 2767415 |
| chr8 | 3308114 | 3308164 |
| chr8 | 3634111 | 3634161 |
| chr8 | 4079638 | 4079688 |
| chr8 | 5240776 | 5240826 |
| chr8 | 6240425 | 6240475 |
| chr8 | 9434670 | 9434720 |
| chr8 | 10057045 | 10057095 |
| chr8 | 10056913 | 10056963 |
| chr8 | 11031481 | 11031531 |
| chr8 | 11303063 | 11303113 |
| chr8 | 13599853 | 13599903 |
| chr8 | 20869616 | 20869666 |
| chr8 | 23489096 | 23489146 |
| chr8 | 24904119 | 24904169 |
| chr8 | 25045804 | 25045854 |
| chr8 | 27315035 | 27315085 |
| chr8 | 28674673 | 28674723 |
| chr11 | 123845138 | 123845188 |
| chr8 | 30185916 | 30185966 |
| chr8 | 30290841 | 30290891 |
| chr11 | 124112011 | 124112061 |
| chr8 | 38650059 | 38650109 |
| chr8 | 39182303 | 39182353 |
| chr8 | 39274628 | 39274678 |
| chr8 | 41478100 | 41478150 |
| chr8 | 42159046 | 42159096 |
| chr8 | 42750055 | 42750105 |
| chr8 | 49135824 | 49135874 |
| chr8 | 50605392 | 50605442 |
| chr8 | 52366310 | 52366360 |
| chr8 | 52367752 | 52367802 |
| chr8 | 52939732 | 52939782 |
| chr8 | 53897879 | 53897929 |
| chr8 | 55474525 | 55474575 |
| chr8 | 55955159 | 55955209 |
| chr8 | 56960287 | 56960337 |
| chr8 | 57833923 | 57833973 |
| chr8 | 58970597 | 58970647 |
| chr8 | 59123588 | 59123638 |
| chr11 | 125483315 | 125483365 |
| chr10 | 18737079 | 18737129 |
| chr8 | 65614653 | 65614703 |
| chr8 | 65969801 | 65969851 |
| chr8 | 67966299 | 67966349 |
| chr8 | 70668833 | 70668883 |
| chr8 | 74178911 | 74178961 |
| chr8 | 74353006 | 74353056 |
| chr11 | 125856573 | 125856623 |
| chr8 | 83188834 | 83188884 |
| chr8 | 88064800 | 88064850 |
| chr8 | 88904237 | 88904287 |
| chr8 | 91427126 | 91427176 |
| chr8 | 92912995 | 92913045 |
| chr8 | 95784891 | 95784941 |
| chr8 | 96906702 | 96906752 |
| chr8 | 97567005 | 97567055 |
| chr8 | 100813728 | 100813778 |
| chr8 | 102481532 | 102481582 |
| chr8 | 105087841 | 105087891 |
| chr8 | 106046427 | 106046477 |
| chr11 | 128338515 | 128338565 |
| chr8 | 107821067 | 107821117 |
| chr11 | 128348352 | 128348402 |
| chr8 | 109802643 | 109802693 |
| chr8 | 110811751 | 110811801 |
| chr8 | 112577804 | 112577854 |
| chr8 | 114430185 | 114430235 |
| chr8 | 114930000 | 114930050 |
| chr8 | 117756124 | 117756174 |
| chr8 | 120092048 | 120092098 |
| chr8 | 120118514 | 120118564 |
| chr8 | 120932954 | 120933004 |
| chr8 | 127699054 | 127699104 |
| chr8 | 128354167 | 128354217 |
| chr8 | 130989970 | 130990020 |
| chr8 | 131776504 | 131776554 |
| chr8 | 132739701 | 132739751 |
| chr8 | 134719620 | 134719670 |
| chr8 | 134849448 | 134849498 |
| chr8 | 135927902 | 135927952 |
| chr8 | 136829502 | 136829552 |
| chr8 | 140613285 | 140613335 |
| chr8 | 141098143 | 141098193 |
| chr8 | 142881525 | 142881575 |
| chr8 | 142939718 | 142939768 |
| chr8 | 142990309 | 142990359 |
| chr8 | 143357322 | 143357372 |
| chr8 | 143858253 | 143858303 |
| chr8 | 144016000 | 144016050 |
| chr8 | 144015752 | 144015802 |
| chr8 | 144384107 | 144384157 |
| chr8 | 144689582 | 144689632 |
| chr8 | 144732070 | 144732120 |
| chr8 | 145327350 | 145327400 |
| chr8 | 145861459 | 145861509 |
| chr8 | 145974883 | 145974933 |
| chr11 | 131168592 | 131168642 |
| chr9 | 799204 | 799254 |
| chr9 | 6684291 | 6684341 |
| chr9 | 6904458 | 6904508 |
| chr9 | 7118221 | 7118271 |
| chr9 | 7118120 | 7118170 |
| chr9 | 12967494 | 12967544 |
| chr9 | 15256636 | 15256686 |
| chr9 | 15799243 | 15799293 |
| chr9 | 16390427 | 16390477 |
| chr9 | 22853319 | 22853369 |
| chr9 | 24561548 | 24561598 |
| chr11 | 133125404 | 133125454 |
| chr9 | 33687572 | 33687622 |
| chr9 | 33687318 | 33687368 |
| chr9 | 36364756 | 36364806 |
| chr9 | 38368829 | 38368879 |
| chr9 | 39044443 | 39044493 |
| chr9 | 43277675 | 43277725 |
| chr9 | 43374178 | 43374228 |
| chr9 | 69765980 | 69766030 |
| chr9 | 70180374 | 70180424 |
| chr9 | 70386210 | 70386260 |
| chr9 | 70493932 | 70493982 |
| chr9 | 70600222 | 70600272 |
| chr9 | 77354998 | 77355048 |
| chr9 | 78819849 | 78819899 |
| chr9 | 83083855 | 83083905 |
| chr9 | 86017377 | 86017427 |
| chr9 | 87313580 | 87313630 |
| chr9 | 87315133 | 87315183 |
| chr9 | 87390311 | 87390361 |
| chr9 | 89910194 | 89910244 |
| chr10 | 20824768 | 20824818 |
| chr11 | 134025879 | 134025929 |
| chr11 | 134038896 | 134038946 |
| chr11 | 134039170 | 134039220 |
| chr11 | 134039734 | 134039784 |
| chr9 | 90776925 | 90776975 |
| chr9 | 90777231 | 90777281 |
| chr9 | 90809618 | 90809668 |
| chr9 | 91878885 | 91878935 |
| chr9 | 92223237 | 92223287 |
| chr9 | 93488821 | 93488871 |
| chr9 | 94257525 | 94257575 |
| chr9 | 94257588 | 94257638 |
| chr9 | 94309238 | 94309288 |
| chr9 | 96417384 | 96417434 |
| chr9 | 98259583 | 98259633 |
| chr9 | 98522581 | 98522631 |
| chr9 | 102019993 | 102020043 |
| chr9 | 103295004 | 103295054 |
| chr9 | 104801964 | 104802014 |
| chr9 | 107430323 | 107430373 |
| chr9 | 110621683 | 110621733 |
| chr9 | 110866344 | 110866394 |
| chr9 | 110867319 | 110867369 |
| chr9 | 112124437 | 112124487 |
| chr9 | 113083066 | 113083116 |
| chr9 | 116859020 | 116859070 |
| chr9 | 117091728 | 117091778 |
| chr9 | 117148295 | 117148345 |
| chr9 | 121197902 | 121197952 |
| chr12 | 1393540 | 1393590 |
| chr9 | 122288132 | 122288182 |
| chr12 | 1537752 | 1537802 |
| chr12 | 1538352 | 1538402 |
| chr9 | 126557199 | 126557249 |
| chr9 | 127713084 | 127713134 |
| chr9 | 127937322 | 127937372 |
| chr9 | 129403868 | 129403918 |
| chr9 | 129467695 | 129467745 |
| chr9 | 129519829 | 129519879 |
| chr9 | 130641227 | 130641277 |
| chr9 | 133107744 | 133107794 |
| chr9 | 133171727 | 133171777 |
| chr9 | 133500141 | 133500191 |
| chr9 | 133711537 | 133711587 |
| chr9 | 133744111 | 133744161 |
| chr9 | 134053809 | 134053859 |
| chr9 | 134955317 | 134955367 |
| chr9 | 135252594 | 135252644 |
| chr9 | 135800449 | 135800499 |
| chr12 | 3690294 | 3690344 |
| chr12 | 4313314 | 4313364 |
| chr10 | 22196439 | 22196489 |
| chr12 | 7789761 | 7789811 |
| chr12 | 7796596 | 7796646 |
| chr12 | 7797555 | 7797605 |
| chr12 | 7919404 | 7919454 |
| chr12 | 8556416 | 8556466 |
| chr12 | 12333686 | 12333736 |
| chr12 | 12471664 | 12471714 |
| chr10 | 1096642 | 1096692 |
| chr12 | 14332264 | 14332314 |
| chr12 | 14363928 | 14363978 |
| chr12 | 14780592 | 14780642 |
| chr12 | 16166769 | 16166819 |
| chr12 | 17253795 | 17253845 |
| chr12 | 19578326 | 19578376 |
| chr12 | 21053440 | 21053490 |
| chr12 | 22743767 | 22743817 |
| chr12 | 22799996 | 22800046 |
| chr12 | 31882106 | 31882156 |
| chr12 | 32267328 | 32267378 |
| chr12 | 41040841 | 41040891 |
| chr12 | 42135594 | 42135644 |
| chr12 | 43822556 | 43822606 |
| chr12 | 45596122 | 45596172 |
| chr12 | 46097142 | 46097192 |
| chr12 | 47317207 | 47317257 |
| chr12 | 47327056 | 47327106 |
| chr12 | 50562628 | 50562678 |
| chr10 | 26904179 | 26904229 |
| chr10 | 27192759 | 27192809 |
| chr12 | 58234923 | 58234973 |
| chr12 | 58809799 | 58809849 |
| chr12 | 64256465 | 64256515 |
| chr10 | 28812081 | 28812131 |
| chr12 | 72034788 | 72034838 |
| chr12 | 73685801 | 73685851 |
| chr10 | 1205870 | 1205920 |
| chr12 | 82119256 | 82119306 |
| chr10 | 29300672 | 29300722 |
| chr12 | 90875344 | 90875394 |
| chr12 | 92519973 | 92520023 |
| chr12 | 92942700 | 92942750 |
| chr12 | 94972803 | 94972853 |
| chr12 | 94983620 | 94983670 |
| chr12 | 95001476 | 95001526 |
| chr12 | 95050865 | 95050915 |
| chr12 | 97508857 | 97508907 |
| chr12 | 98664696 | 98664746 |
| chr12 | 100317739 | 100317789 |
| chr12 | 100461814 | 100461864 |
| chr12 | 101929708 | 101929758 |
| chr12 | 103145870 | 103145920 |
| chr12 | 103326196 | 103326246 |
| chr12 | 103874168 | 103874218 |
| chr12 | 105215436 | 105215486 |
| chr12 | 107378215 | 107378265 |
| chr12 | 115473906 | 115473956 |
| chr12 | 118776381 | 118776431 |
| chr12 | 119868187 | 119868237 |
| chr12 | 120116182 | 120116232 |
| chr12 | 120443279 | 120443329 |
| chr12 | 123476318 | 123476368 |
| chr12 | 123480952 | 123481002 |
| chr12 | 123569190 | 123569240 |
| chr12 | 124245896 | 124245946 |
| chr12 | 124539849 | 124539899 |
| chr12 | 127623242 | 127623292 |
| chr12 | 128713301 | 128713351 |
| chr12 | 129761044 | 129761094 |
| chr12 | 130366071 | 130366121 |
| chr12 | 131416057 | 131416107 |
| chr10 | 35399720 | 35399770 |
| chr12 | 131786851 | 131786901 |
| chr12 | 131844408 | 131844458 |
| chr12 | 131999569 | 131999619 |
| chr13 | 18993068 | 18993118 |
| chr13 | 19148845 | 19148895 |
| chr13 | 19476914 | 19476964 |
| chr13 | 19482651 | 19482701 |
| chr13 | 19868779 | 19868829 |
| chr13 | 20715923 | 20715973 |
| chr13 | 21333662 | 21333712 |
| chr13 | 21350492 | 21350542 |
| chr13 | 22480241 | 22480291 |
| chr13 | 22758755 | 22758805 |
| chr13 | 22758704 | 22758754 |
| chr13 | 23199912 | 23199962 |
| chr10 | 36658059 | 36658109 |
| chr13 | 25078283 | 25078333 |
| chr13 | 25269115 | 25269165 |
| chr13 | 25305442 | 25305492 |
| chr13 | 25865336 | 25865386 |
| chr13 | 27182945 | 27182995 |
| chr13 | 28623492 | 28623542 |
| chr13 | 28962663 | 28962713 |
| chr13 | 29035450 | 29035500 |
| chr13 | 30564190 | 30564240 |
| chr13 | 37476619 | 37476669 |
| chr13 | 38728265 | 38728315 |
| chr13 | 39176590 | 39176640 |
| chr13 | 40377103 | 40377153 |
| chr13 | 41181593 | 41181643 |
| chr10 | 37995435 | 37995485 |
| chr10 | 37995149 | 37995199 |
| chr13 | 43459903 | 43459953 |
| chr13 | 45923400 | 45923450 |
| chr13 | 46216481 | 46216531 |
| chr13 | 49834829 | 49834879 |
| chr13 | 49862648 | 49862698 |
| chr13 | 50371988 | 50372038 |
| chr13 | 53707291 | 53707341 |
| chr13 | 57341338 | 57341388 |
| chr10 | 42366038 | 42366088 |
| chr13 | 60997521 | 60997571 |
| chr13 | 61283907 | 61283957 |
| chr13 | 61389286 | 61389336 |
| chr13 | 62274430 | 62274480 |
| chr13 | 64066618 | 64066668 |
| chr13 | 69134246 | 69134296 |
| chr13 | 71873223 | 71873273 |
| chr10 | 42732349 | 42732399 |
| chr13 | 76746786 | 76746836 |
| chr13 | 77836863 | 77836913 |
| chr13 | 78655728 | 78655778 |
| chr13 | 83848301 | 83848351 |
| chr13 | 85240987 | 85241037 |
| chr13 | 85532201 | 85532251 |
| chr13 | 86771074 | 86771124 |
| chr13 | 90782739 | 90782789 |
| chr13 | 91645966 | 91646016 |
| chr13 | 93019902 | 93019952 |
| chr13 | 95392261 | 95392311 |
| chr13 | 97969087 | 97969137 |
| chr13 | 98062290 | 98062340 |
| chr13 | 104745758 | 104745808 |
| chr13 | 104805482 | 104805532 |
| chr13 | 104920898 | 104920948 |
| chr13 | 106587316 | 106587366 |
| chr13 | 106634365 | 106634415 |
| chr13 | 107448770 | 107448820 |
| chr13 | 108580440 | 108580490 |
| chr13 | 109616557 | 109616607 |
| chr10 | 44113143 | 44113193 |
| chr13 | 109758072 | 109758122 |
| chr13 | 110957396 | 110957446 |
| chr13 | 111622964 | 111623014 |
| chr13 | 111829903 | 111829953 |
| chr10 | 45227712 | 45227762 |
| chr14 | 19251435 | 19251485 |
| chr14 | 21708164 | 21708214 |
| chr10 | 45441452 | 45441502 |
| chr14 | 21916565 | 21916615 |
| chr14 | 22116007 | 22116057 |
| chr14 | 25625906 | 25625956 |
| chr14 | 25650268 | 25650318 |
| chr14 | 27133030 | 27133080 |
| chr14 | 29276004 | 29276054 |
| chr14 | 30769360 | 30769410 |
| chr14 | 34104664 | 34104714 |
| chr14 | 37654106 | 37654156 |
| chr14 | 38604806 | 38604856 |
| chr14 | 41747354 | 41747404 |
| chr14 | 43346977 | 43347027 |
| chr14 | 43347312 | 43347362 |
| chr14 | 45968465 | 45968515 |
| chr14 | 46220196 | 46220246 |
| chr14 | 49928480 | 49928530 |
| chr14 | 51269886 | 51269936 |
| chr10 | 50093323 | 50093373 |
| chr14 | 55134116 | 55134166 |
| chr14 | 56597995 | 56598045 |
| chr14 | 56598159 | 56598209 |
| chr14 | 63719275 | 63719325 |
| chr14 | 63901595 | 63901645 |
| chr10 | 51080674 | 51080724 |
| chr14 | 69426430 | 69426480 |
| chr14 | 69600075 | 69600125 |
| chr14 | 69599905 | 69599955 |
| chr14 | 73939807 | 73939857 |
| chr14 | 74199890 | 74199940 |
| chr14 | 75534603 | 75534653 |
| chr14 | 76569795 | 76569845 |
| chr14 | 77026322 | 77026372 |
| chr14 | 78750139 | 78750189 |
| chr14 | 79275972 | 79276022 |
| chr14 | 79478772 | 79478822 |
| chr14 | 80544929 | 80544979 |
| chr14 | 80976521 | 80976571 |
| chr14 | 81678713 | 81678763 |
| chr14 | 82176040 | 82176090 |
| chr14 | 82569731 | 82569781 |
| chr14 | 83710423 | 83710473 |
| chr14 | 84765513 | 84765563 |
| chr14 | 87363378 | 87363428 |
| chr14 | 87588979 | 87589029 |
| chr14 | 87661952 | 87662002 |
| chr14 | 87748580 | 87748630 |
| chr14 | 88168864 | 88168914 |
| chr14 | 88949531 | 88949581 |
| chr14 | 89568308 | 89568358 |
| chr14 | 90848986 | 90849036 |
| chr14 | 90849827 | 90849877 |
| chr14 | 91044551 | 91044601 |
| chr10 | 55300999 | 55301049 |
| chr14 | 92327056 | 92327106 |
| chr14 | 94017472 | 94017522 |
| chr14 | 99214087 | 99214137 |
| chr14 | 99700013 | 99700063 |
| chr14 | 100122939 | 100122989 |
| chr14 | 100122772 | 100122822 |
| chr14 | 100396278 | 100396328 |
| chr14 | 101031609 | 101031659 |
| chr10 | 59346366 | 59346416 |
| chr14 | 102314118 | 102314168 |
| chr14 | 102853647 | 102853697 |
| chr14 | 102853592 | 102853642 |
| chr14 | 103308182 | 103308232 |
| chr14 | 103742588 | 103742638 |
| chr14 | 103973354 | 103973404 |
| chr14 | 104031130 | 104031180 |
| chr14 | 104274141 | 104274191 |
| chr14 | 104288916 | 104288966 |
| chr15 | 18396801 | 18396851 |
| chr15 | 20432757 | 20432807 |
| chr15 | 20765506 | 20765556 |
| chr15 | 22700248 | 22700298 |
| chr15 | 23495470 | 23495520 |
| chr10 | 61880041 | 61880091 |
| chr15 | 27394543 | 27394593 |
| chr15 | 29356786 | 29356836 |
| chr15 | 29413401 | 29413451 |
| chr15 | 29663999 | 29664049 |
| chr15 | 33733022 | 33733072 |
| chr15 | 36341117 | 36341167 |
| chr15 | 36340993 | 36341043 |
| chr15 | 42399105 | 42399155 |
| chr10 | 2527319 | 2527369 |
| chr15 | 51424299 | 51424349 |
| chr15 | 52039484 | 52039534 |
| chr15 | 53501071 | 53501121 |
| chr15 | 53600647 | 53600697 |
| chr10 | 65903931 | 65903981 |
| chr15 | 59826949 | 59826999 |
| chr15 | 62068896 | 62068946 |
| chr15 | 67367632 | 67367682 |
| chr15 | 76656241 | 76656291 |
| chr15 | 79422219 | 79422269 |
| chr15 | 79872020 | 79872070 |
| chr15 | 80102059 | 80102109 |
| chr15 | 80119673 | 80119723 |
| chr15 | 81315284 | 81315334 |
| chr15 | 81314781 | 81314831 |
| chr15 | 83293665 | 83293715 |
| chr15 | 84306899 | 84306949 |
| chr15 | 85052003 | 85052053 |
| chr15 | 88380808 | 88380858 |
| chr15 | 91705653 | 91705703 |
| chr15 | 96990183 | 96990233 |
| chr15 | 98237763 | 98237813 |
| chr15 | 98251702 | 98251752 |
| chr16 | 411232 | 411282 |
| chr16 | 1075700 | 1075750 |
| chr16 | 1167543 | 1167593 |
| chr16 | 1167367 | 1167417 |
| chr16 | 1273475 | 1273525 |
| chr16 | 2875784 | 2875834 |
| chr16 | 4044254 | 4044304 |
| chr16 | 5248174 | 5248224 |
| chr16 | 5380161 | 5380211 |
| chr16 | 5834873 | 5834923 |
| chr16 | 6084208 | 6084258 |
| chr16 | 7315657 | 7315707 |
| chr16 | 7512964 | 7513014 |
| chr16 | 8348374 | 8348424 |
| chr16 | 8348115 | 8348165 |
| chr10 | 71196312 | 71196362 |
| chr10 | 71196111 | 71196161 |
| chr16 | 9082351 | 9082401 |
| chr16 | 9240077 | 9240127 |
| chr16 | 9635214 | 9635264 |
| chr16 | 11859406 | 11859456 |
| chr16 | 15589703 | 15589753 |
| chr16 | 17931782 | 17931832 |
| chr16 | 18013410 | 18013460 |
| chr16 | 18921356 | 18921406 |
| chr10 | 71624333 | 71624383 |
| chr16 | 19734793 | 19734843 |
| chr16 | 21293295 | 21293345 |
| chr16 | 21615688 | 21615738 |
| chr16 | 22903640 | 22903690 |
| chr16 | 24017450 | 24017500 |
| chr16 | 24314443 | 24314493 |
| chr10 | 72105072 | 72105122 |
| chr16 | 25664752 | 25664802 |
| chr16 | 28208462 | 28208512 |
| chr16 | 28314359 | 28314409 |
| chr16 | 29276055 | 29276105 |
| chr16 | 46961913 | 46961963 |
| chr16 | 48167565 | 48167615 |
| chr16 | 48167453 | 48167503 |
| chr16 | 52807808 | 52807858 |
| chr16 | 53693173 | 53693223 |
| chr16 | 55890375 | 55890425 |
| chr16 | 56523367 | 56523417 |
| chr16 | 58244086 | 58244136 |
| chr16 | 58597482 | 58597532 |
| chr16 | 58995414 | 58995464 |
| chr16 | 61793372 | 61793422 |
| chr16 | 62315749 | 62315799 |
| chr16 | 63485274 | 63485324 |
| chr16 | 65564315 | 65564365 |
| chr16 | 66497775 | 66497825 |
| chr16 | 66762598 | 66762648 |
| chr10 | 73864084 | 73864134 |
| chr10 | 74052020 | 74052070 |
| chr16 | 73886506 | 73886556 |
| chr16 | 73886385 | 73886435 |
| chr16 | 73987014 | 73987064 |
| chr16 | 75279095 | 75279145 |
| chr16 | 75333135 | 75333185 |
| chr16 | 75401787 | 75401837 |
| chr16 | 77291255 | 77291305 |
| chr16 | 77589290 | 77589340 |
| chr16 | 80121277 | 80121327 |
| chr16 | 80121150 | 80121200 |
| chr16 | 80193745 | 80193795 |
| chr16 | 80281369 | 80281419 |
| chr16 | 80724640 | 80724690 |
| chr16 | 81356174 | 81356224 |
| chr16 | 82071310 | 82071360 |
| chr16 | 82742980 | 82743030 |
| chr10 | 74814408 | 74814458 |
| chr10 | 74814672 | 74814722 |
| chr10 | 3305135 | 3305185 |
| chr10 | 3304915 | 3304965 |
| chr16 | 88006803 | 88006853 |
| chr16 | 88373737 | 88373787 |
| chr16 | 89155353 | 89155403 |
| chr16 | 89155217 | 89155267 |
| chr10 | 75790824 | 75790874 |
| chr10 | 75790960 | 75791010 |
| chr17 | 259399 | 259449 |
| chr17 | 1436954 | 1437004 |
| chr17 | 6279672 | 6279722 |
| chr17 | 6280400 | 6280450 |
| chr17 | 7160274 | 7160324 |
| chr10 | 77704876 | 77704926 |
| chr17 | 8456399 | 8456449 |
| chr17 | 8753813 | 8753863 |
| chr17 | 9077327 | 9077377 |
| chr17 | 10116628 | 10116678 |
| chr17 | 10132106 | 10132156 |
| chr10 | 78848227 | 78848277 |
| chr17 | 11179830 | 11179880 |
| chr17 | 12321590 | 12321640 |
| chr17 | 15823865 | 15823915 |
| chr17 | 16021779 | 16021829 |
| chr17 | 16589602 | 16589652 |
| chr17 | 18187997 | 18188047 |
| chr10 | 79356589 | 79356639 |
| chr10 | 79362760 | 79362810 |
| chr17 | 18370892 | 18370942 |
| chr17 | 19016137 | 19016187 |
| chr17 | 19486515 | 19486565 |
| chr17 | 27799718 | 27799768 |
| chr17 | 32249238 | 32249288 |

Table 15CZ. Repeat Category MLT1A0

| | | |
|---|---|---|
| chr10 | 81611942 | 81611992 |
| chr10 | 83225068 | 83225118 |
| chr17 | 46976620 | 46976670 |
| chr17 | 53252158 | 53252208 |
| chr17 | 61210321 | 61210371 |
| chr17 | 62949457 | 62949507 |
| chr17 | 64026522 | 64026572 |
| chr17 | 70642640 | 70642690 |
| chr17 | 71736042 | 71736092 |
| chr17 | 73232506 | 73232556 |
| chr17 | 75443452 | 75443502 |
| chr17 | 75613512 | 75613562 |
| chr17 | 76177819 | 76177869 |
| chr17 | 76889972 | 76890022 |
| chr17 | 77230554 | 77230604 |
| chr17 | 77230477 | 77230527 |
| chr17 | 78579280 | 78579330 |
| chr17 | 79310647 | 79310697 |
| chr10 | 93320659 | 93320709 |
| chr17 | 81683706 | 81683756 |
| chr18 | 3519165 | 3519215 |
| chr18 | 3518978 | 3519028 |
| chr18 | 4959385 | 4959435 |
| chr18 | 7381839 | 7381889 |
| chr10 | 94722546 | 94722596 |
| chr18 | 13833454 | 13833504 |
| chr18 | 13935237 | 13935287 |
| chr18 | 17913844 | 17913894 |
| chr18 | 19016706 | 19016756 |
| chr18 | 19236242 | 19236292 |
| chr18 | 20671551 | 20671601 |
| chr18 | 21836914 | 21836964 |
| chr18 | 25074663 | 25074713 |
| chr10 | 96724400 | 96724450 |
| chr18 | 45244783 | 45244833 |
| chr18 | 46493576 | 46493626 |
| chr18 | 46493517 | 46493567 |
| chr18 | 48923753 | 48923803 |
| chr18 | 51774084 | 51774134 |
| chr18 | 53456341 | 53456391 |
| chr18 | 57656031 | 57656081 |
| chr18 | 60409439 | 60409489 |
| chr18 | 63723225 | 63723275 |
| chr18 | 64694846 | 64694896 |
| chr18 | 66218240 | 66218290 |
| chr18 | 67167446 | 67167496 |
| chr18 | 67364975 | 67365025 |
| chr18 | 67364821 | 67364871 |
| chr18 | 68594111 | 68594161 |
| chr18 | 69892343 | 69892393 |
| chr18 | 70215324 | 70215374 |
| chr18 | 70615032 | 70615082 |
| chr18 | 71939462 | 71939512 |
| chr18 | 74012476 | 74012526 |
| chr18 | 74786938 | 74786988 |
| chr18 | 74808282 | 74808332 |
| chr19 | 310891 | 310941 |
| chr19 | 3056906 | 3056956 |
| chr19 | 8759702 | 8759752 |
| chr19 | 13535918 | 13535968 |
| chr19 | 16646692 | 16646742 |
| chr19 | 16647855 | 16647905 |
| chr19 | 16758682 | 16758732 |
| chr19 | 16771851 | 16771901 |
| chr19 | 16771760 | 16771810 |
| chr19 | 16941728 | 16941778 |
| chr19 | 34809321 | 34809371 |
| chr19 | 35909737 | 35909787 |
| chr19 | 36311614 | 36311664 |
| chr19 | 36311413 | 36311463 |
| chr19 | 38965314 | 38965364 |
| chr19 | 45365521 | 45365571 |
| chr19 | 45365588 | 45365638 |
| chr19 | 49178403 | 49178453 |
| chr19 | 49361007 | 49361057 |
| chr19 | 49788468 | 49788518 |
| chr10 | 108303197 | 108303247 |
| chr10 | 110043280 | 110043330 |
| chr19 | 52481083 | 52481133 |
| chr19 | 53209375 | 53209425 |
| chr19 | 53434483 | 53434533 |
| chr10 | 112161847 | 112161897 |
| chr19 | 55268566 | 55268616 |
| chr19 | 56930240 | 56930290 |
| chr19 | 57678883 | 57678933 |
| chr19 | 58129969 | 58130019 |
| chr19 | 61880909 | 61880959 |
| chr19 | 62143366 | 62143416 |
| chr19 | 62191428 | 62191478 |
| chr1 | 3239901 | 3239951 |
| chr1 | 5617136 | 5617186 |
| chr1 | 7026200 | 7026250 |
| chr1 | 8611849 | 8611899 |
| chr1 | 11676008 | 11676058 |
| chr1 | 17194457 | 17194507 |
| chr1 | 18582376 | 18582426 |
| chr1 | 21199715 | 21199765 |
| chr1 | 23457487 | 23457537 |
| chr10 | 120277329 | 120277379 |
| chr10 | 120915467 | 120915517 |
| chr1 | 35705258 | 35705308 |
| chr10 | 121029097 | 121029147 |
| chr10 | 121188378 | 121188428 |
| chr1 | 42245641 | 42245691 |
| chr1 | 45126460 | 45126510 |
| chr1 | 52401142 | 52401192 |
| chr1 | 54347852 | 54347902 |
| chr1 | 55771665 | 55771715 |
| chr1 | 60438978 | 60439028 |
| chr1 | 63061742 | 63061792 |
| chr1 | 63172408 | 63172458 |
| chr1 | 63172931 | 63172981 |
| chr1 | 64956952 | 64957002 |
| chr1 | 66943454 | 66943504 |
| chr1 | 67020448 | 67020498 |
| chr1 | 78659185 | 78659235 |
| chr1 | 79154137 | 79154187 |
| chr1 | 81416608 | 81416658 |
| chr1 | 81484641 | 81484691 |
| chr1 | 82025148 | 82025198 |
| chr1 | 82298900 | 82298950 |
| chr1 | 83268350 | 83268400 |
| chr1 | 83577899 | 83577949 |
| chr1 | 87979260 | 87979310 |
| chr1 | 92765587 | 92765637 |
| chr1 | 94948623 | 94948673 |
| chr1 | 100110375 | 100110425 |
| chr1 | 103414281 | 103414331 |
| chr10 | 125889077 | 125889127 |
| chr1 | 107820276 | 107820326 |
| chr1 | 110287077 | 110287127 |
| chr1 | 112507004 | 112507054 |
| chr1 | 112828223 | 112828273 |
| chr1 | 112882515 | 112882565 |
| chr1 | 115095593 | 115095643 |
| chr10 | 126349833 | 126349883 |
| chr1 | 146651960 | 146652010 |
| chr1 | 146891346 | 146891396 |
| chr10 | 7179541 | 7179591 |
| chr10 | 128186662 | 128186712 |
| chr1 | 157294714 | 157294764 |
| chr1 | 158747332 | 158747382 |
| chr1 | 162893018 | 162893068 |
| chr1 | 163017873 | 163017923 |
| chr10 | 128667547 | 128667597 |
| chr1 | 171441372 | 171441422 |
| chr1 | 180967122 | 180967172 |
| chr1 | 181386209 | 181386259 |
| chr1 | 188268696 | 188268746 |
| chr1 | 188769788 | 188769838 |
| chr1 | 193859848 | 193859898 |
| chr1 | 195391224 | 195391274 |
| chr1 | 199404186 | 199404236 |
| chr1 | 202793549 | 202793599 |
| chr1 | 208747164 | 208747214 |
| chr1 | 208747294 | 208747344 |
| chr10 | 130770527 | 130770577 |
| chr1 | 211503770 | 211503820 |
| chr1 | 215461268 | 215461318 |
| chr1 | 225092592 | 225092642 |
| chr1 | 225613996 | 225614046 |
| chr1 | 229158965 | 229159015 |
| chr1 | 229999961 | 230000011 |
| chr1 | 230253751 | 230253801 |
| chr1 | 232149826 | 232149876 |
| chr10 | 131784494 | 131784544 |
| chr1 | 234311698 | 234311748 |
| chr1 | 234693440 | 234693490 |
| chr10 | 131821853 | 131821903 |
| chr1 | 237502167 | 237502217 |
| chr1 | 237981750 | 237981800 |
| chr1 | 241978987 | 241979037 |
| chr1 | 242240847 | 242240897 |
| chr1 | 242631008 | 242631058 |
| chr1 | 244499146 | 244499196 |
| chr1 | 244500202 | 244500252 |
| chr10 | 132476331 | 132476381 |
| chr20 | 1310001 | 1310051 |
| chr20 | 1309684 | 1309734 |
| chr20 | 1380176 | 1380226 |
| chr20 | 3456069 | 3456119 |
| chr20 | 5693133 | 5693183 |
| chr10 | 132892314 | 132892364 |
| chr20 | 10419827 | 10419877 |
| chr20 | 18414129 | 18414179 |
| chr20 | 19056353 | 19056403 |
| chr20 | 21785618 | 21785668 |
| chr20 | 25108238 | 25108288 |
| chr20 | 33555072 | 33555122 |
| chr10 | 133682067 | 133682117 |
| chr10 | 133733423 | 133733473 |
| chr20 | 40242328 | 40242378 |
| chr20 | 41723146 | 41723196 |
| chr20 | 43128178 | 43128228 |
| chr20 | 44437862 | 44437912 |
| chr20 | 53441138 | 53441188 |
| chr20 | 56413968 | 56414018 |
| chr20 | 56413906 | 56413956 |
| chr20 | 56864773 | 56864823 |
| chr10 | 134384914 | 134384964 |
| chr20 | 61352702 | 61352752 |
| chr10 | 134647407 | 134647457 |
| chr20 | 62945179 | 62945229 |
| chr20 | 63002219 | 63002269 |
| chr21 | 16313687 | 16313737 |
| chr21 | 17865156 | 17865206 |
| chr21 | 19669574 | 19669624 |
| chr21 | 20057641 | 20057691 |
| chr21 | 21751522 | 21751572 |
| chr21 | 22626598 | 22626648 |
| chr21 | 25249060 | 25249110 |
| chr21 | 27762911 | 27762961 |
| chr21 | 29370672 | 29370722 |
| chr21 | 31662159 | 31662209 |
| chr21 | 32178136 | 32178186 |
| chr21 | 37179223 | 37179273 |
| chr21 | 37597268 | 37597318 |
| chr21 | 39002331 | 39002381 |
| chr21 | 41170710 | 41170760 |
| chr21 | 42526742 | 42526792 |
| chr21 | 43098080 | 43098130 |
| chr21 | 43549330 | 43549380 |
| chr21 | 43555204 | 43555254 |
| chr21 | 43761456 | 43761506 |
| chr21 | 45065655 | 45065705 |
| chr11 | 1044225 | 1044275 |
| chr11 | 1351881 | 1351931 |
| chr22 | 20581099 | 20581149 |
| chr22 | 22238340 | 22238390 |
| chr22 | 23538245 | 23538295 |
| chr22 | 23539287 | 23539337 |
| chr22 | 32607869 | 32607919 |
| chr22 | 33724834 | 33724884 |
| chr22 | 35694072 | 35694122 |
| chr22 | 36169523 | 36169573 |
| chr22 | 36828976 | 36829026 |
| chr22 | 37972796 | 37972846 |
| chr22 | 38370087 | 38370137 |
| chr22 | 40042161 | 40042211 |
| chr11 | 2592794 | 2592844 |
| chr22 | 41232035 | 41232085 |
| chr22 | 43513340 | 43513390 |
| chr22 | 44784989 | 44785039 |
| chr22 | 44900119 | 44900169 |
| chr22 | 44932858 | 44932908 |
| chr22 | 45746292 | 45746342 |
| chr22 | 46227780 | 46227830 |
| chr22 | 46228591 | 46228641 |
| chr22 | 46282690 | 46282740 |
| chr22 | 46817268 | 46817318 |
| chr22 | 47287788 | 47287838 |
| chr22 | 47305008 | 47305058 |
| chr22 | 47417932 | 47417982 |
| chr22 | 47519185 | 47519235 |
| chr22 | 47658403 | 47658453 |
| chr22 | 47790413 | 47790463 |
| chr22 | 47792067 | 47792117 |
| chrX | 3166567 | 3166617 |
| chrX | 3218639 | 3218689 |
| chrX | 5040638 | 5040688 |
| chrX | 6275503 | 6275553 |
| chrX | 9385793 | 9385843 |
| chr11 | 3918268 | 3918318 |
| chrX | 10511435 | 10511485 |
| chrX | 11334801 | 11334851 |
| chrX | 15137566 | 15137616 |
| chrX | 18041565 | 18041615 |
| chrX | 28047694 | 28047744 |
| chrX | 30333009 | 30333059 |
| chr11 | 6298286 | 6298336 |
| chrX | 33923431 | 33923481 |
| chrX | 34424980 | 34425030 |
| chrX | 38745528 | 38745578 |
| chrX | 40347585 | 40347635 |
| chrX | 43354950 | 43355000 |
| chrX | 43564155 | 43564205 |
| chrX | 53120497 | 53120547 |
| chr11 | 8838905 | 8838955 |
| chrX | 96345770 | 96345820 |
| chrX | 96474223 | 96474273 |
| chrX | 111518906 | 111518956 |
| chrX | 112216226 | 112216276 |
| chrX | 117659874 | 117659924 |
| chrX | 118283500 | 118283550 |
| chrX | 120903830 | 120903880 |
| chr11 | 11221798 | 11221848 |
| chr11 | 11571545 | 11571595 |
| chrX | 134676196 | 134676246 |
| chrX | 144073935 | 144073985 |
| chrX | 144677977 | 144678027 |
| chrX | 149399945 | 149399995 |
| chrX | 150652116 | 150652166 |
| chrX | 150651952 | 150652002 |
| chr11 | 13769871 | 13769921 |
| chrY | 3541769 | 3541819 |
| chrY | 9419459 | 9419509 |
| chr2 | 284574 | 284624 |
| chr11 | 15298355 | 15298405 |
| chr11 | 15498379 | 15498429 |
| chr2 | 1521082 | 1521132 |
| chr2 | 2579496 | 2579546 |
| chr2 | 3182823 | 3182873 |
| chr2 | 3346018 | 3346068 |
| chr2 | 5587007 | 5587057 |
| chr2 | 9840685 | 9840735 |
| chr2 | 14754314 | 14754364 |
| chr2 | 24564515 | 24564565 |
| chr2 | 28114831 | 28114881 |
| chr11 | 18716030 | 18716080 |
| chr2 | 30350587 | 30350637 |
| chr2 | 30522311 | 30522361 |
| chr2 | 31095126 | 31095176 |
| chr2 | 32903249 | 32903299 |
| chr2 | 36348079 | 36348129 |
| chr2 | 38102124 | 38102174 |
| chr10 | 636818 | 636868 |
| chr2 | 42217298 | 42217348 |
| chr2 | 43183865 | 43183915 |
| chr2 | 48767006 | 48767056 |
| chr2 | 48804122 | 48804172 |
| chr2 | 51905688 | 51905738 |
| chr11 | 22125717 | 22125767 |
| chr11 | 22421686 | 22421736 |
| chr2 | 56326920 | 56326970 |
| chr2 | 58927586 | 58927636 |
| chr2 | 59654211 | 59654261 |
| chr2 | 64804357 | 64804407 |
| chr2 | 69495081 | 69495131 |
| chr2 | 69834922 | 69834972 |
| chr10 | 11805059 | 11805109 |
| chr2 | 71293417 | 71293467 |
| chr2 | 72415843 | 72415893 |
| chr2 | 80164377 | 80164427 |
| chr2 | 86755280 | 86755330 |
| chr2 | 87312463 | 87312513 |
| chr2 | 94867598 | 94867648 |
| chr2 | 100216551 | 100216601 |
| chr2 | 100216389 | 100216439 |
| chr2 | 100891723 | 100891773 |
| chr2 | 101033894 | 101033944 |
| chr2 | 101665805 | 101665855 |
| chr2 | 104406101 | 104406151 |
| chr2 | 105696530 | 105696580 |
| chr2 | 109484189 | 109484239 |
| chr2 | 112131838 | 112131888 |
| chr2 | 112539179 | 112539229 |
| chr11 | 32769707 | 32769757 |
| chr2 | 128909857 | 128909907 |
| chr2 | 129058858 | 129058908 |
| chr2 | 135600950 | 135601000 |
| chr2 | 140885458 | 140885508 |
| chr2 | 150406171 | 150406221 |
| chr2 | 152070570 | 152070620 |
| chr2 | 153448657 | 153448707 |
| chr2 | 158561622 | 158561672 |
| chr2 | 158893522 | 158893572 |
| chr2 | 159728955 | 159729005 |
| chr2 | 159970429 | 159970479 |
| chr2 | 164354863 | 164354913 |
| chr2 | 168567188 | 168567238 |
| chr2 | 174875027 | 174875077 |
| chr2 | 177276011 | 177276061 |
| chr2 | 185274141 | 185274191 |
| chr2 | 186997185 | 186997235 |
| chr2 | 188311216 | 188311266 |
| chr2 | 190360894 | 190360944 |
| chr2 | 190464390 | 190464440 |
| chr11 | 39073247 | 39073297 |
| chr2 | 198419777 | 198419827 |
| chr2 | 201863185 | 201863235 |
| chr2 | 201902010 | 201902060 |
| chr2 | 203573852 | 203573902 |
| chr2 | 204818883 | 204818933 |
| chr2 | 209445754 | 209445804 |
| chr2 | 210058441 | 210058491 |
| chr2 | 213618425 | 213618475 |
| chr2 | 215419112 | 215419162 |
| chr2 | 215921092 | 215921142 |
| chr2 | 216626792 | 216626842 |
| chr2 | 217592477 | 217592527 |
| chr11 | 44047219 | 44047269 |
| chr2 | 225515562 | 225515612 |
| chr2 | 226175822 | 226175872 |
| chr2 | 233997498 | 233997548 |
| chr2 | 235714871 | 235714921 |
| chr2 | 236948295 | 236948345 |
| chr2 | 236948579 | 236948629 |
| chr2 | 237300901 | 237300951 |
| chr2 | 238858390 | 238858440 |
| chr11 | 45798215 | 45798265 |
| chr2 | 239749814 | 239749864 |
| chr2 | 239750109 | 239750159 |
| chr2 | 241504874 | 241504924 |
| chr2 | 241587406 | 241587456 |
| chr2 | 241633758 | 241633808 |
| chr2 | 243080706 | 243080756 |
| chr3 | 2811950 | 2812000 |
| chr3 | 3022512 | 3022562 |
| chr3 | 4580965 | 4581015 |
| chr3 | 5594478 | 5594528 |
| chr3 | 5904818 | 5904868 |
| chr3 | 6243256 | 6243306 |
| chr3 | 10747574 | 10747624 |
| chr3 | 11791248 | 11791298 |
| chr3 | 14935771 | 14935821 |
| chr3 | 17131930 | 17131980 |
| chr3 | 23902000 | 23902050 |
| chr3 | 27754063 | 27754113 |
| chr3 | 29132794 | 29132844 |
| chr3 | 30028531 | 30028581 |
| chr3 | 31851455 | 31851505 |
| chr3 | 32811215 | 32811265 |
| chr3 | 32811877 | 32811927 |
| chr3 | 35375834 | 35375884 |
| chr3 | 37186933 | 37186983 |
| chr3 | 39256408 | 39256458 |
| chr3 | 45589721 | 45589771 |
| chr3 | 45658418 | 45658468 |
| chr3 | 47234755 | 47234805 |
| chr3 | 52983102 | 52983152 |
| chr3 | 63618129 | 63618179 |
| chr3 | 69035014 | 69035064 |
| chr11 | 58010587 | 58010637 |
| chr3 | 76894807 | 76894857 |
| chr3 | 81149478 | 81149528 |
| chr3 | 86019685 | 86019735 |
| chr3 | 88446965 | 88447015 |
| chr3 | 89351303 | 89351353 |
| chr3 | 97063933 | 97063983 |
| chr3 | 101444828 | 101444878 |
| chr3 | 102658141 | 102658191 |
| chr3 | 103556591 | 103556641 |
| chr3 | 104602332 | 104602382 |
| chr3 | 114991901 | 114991951 |
| chr3 | 116814508 | 116814558 |
| chr3 | 119211147 | 119211197 |
| chr3 | 119212344 | 119212394 |
| chr3 | 120426357 | 120426407 |
| chr10 | 12768049 | 12768099 |
| chr3 | 130776902 | 130776952 |
| chr3 | 135433419 | 135433469 |
| chr3 | 143039830 | 143039880 |
| chr3 | 144547246 | 144547296 |
| chr3 | 145789345 | 145789395 |
| chr3 | 145790891 | 145790941 |
| chr3 | 151733942 | 151733992 |
| chr3 | 152324205 | 152324255 |
| chr3 | 154879078 | 154879128 |
| chr3 | 156099843 | 156099893 |
| chr3 | 161184707 | 161184757 |
| chr3 | 163162516 | 163162566 |
| chr3 | 172967705 | 172967755 |
| chr3 | 175561778 | 175561828 |
| chr3 | 176021815 | 176021865 |
| chr3 | 180143027 | 180143077 |
| chr3 | 182488050 | 182488100 |
| chr3 | 184502437 | 184502487 |
| chr3 | 184728547 | 184728597 |
| chr3 | 187066477 | 187066527 |
| chr3 | 187870294 | 187870344 |
| chr3 | 194414717 | 194414767 |
| chr4 | 697614 | 697664 |
| chr4 | 909006 | 909056 |
| chr4 | 3680591 | 3680641 |
| chr4 | 4652175 | 4652225 |
| chr4 | 4651920 | 4651970 |
| chr4 | 5471508 | 5471558 |
| chr4 | 6621795 | 6621845 |
| chr4 | 6691082 | 6691132 |
| chr4 | 6692774 | 6692824 |
| chr4 | 7003045 | 7003095 |
| chr4 | 7224898 | 7224948 |
| chr4 | 8323576 | 8323626 |
| chr4 | 8448890 | 8448940 |
| chr4 | 10581814 | 10581864 |
| chr4 | 13422068 | 13422118 |
| chr4 | 13607805 | 13607855 |
| chr4 | 18652069 | 18652119 |
| chr4 | 19174707 | 19174757 |
| chr4 | 19715211 | 19715261 |
| chr4 | 26863646 | 26863696 |
| chr4 | 28966347 | 28966397 |
| chr4 | 31399089 | 31399139 |
| chr4 | 37033869 | 37033919 |
| chr4 | 38628292 | 38628342 |
| chr4 | 38826368 | 38826418 |
| chr4 | 39294338 | 39294388 |
| chr4 | 42992684 | 42992734 |
| chr4 | 45519823 | 45519873 |
| chr4 | 47493774 | 47493824 |
| chr4 | 53871027 | 53871077 |
| chr4 | 55268569 | 55268619 |
| chr4 | 56995897 | 56995947 |
| chr4 | 57138589 | 57138639 |
| chr4 | 57552554 | 57552604 |
| chr11 | 65286404 | 65286454 |
| chr4 | 63361730 | 63361780 |
| chr4 | 63602380 | 63602430 |
| chr4 | 65749782 | 65749832 |
| chr4 | 67171406 | 67171456 |
| chr4 | 67552484 | 67552534 |
| chr4 | 76836604 | 76836654 |
| chr4 | 84167857 | 84167907 |
| chr4 | 94193263 | 94193313 |
| chr4 | 97351441 | 97351491 |
| chr4 | 101492283 | 101492333 |
| chr4 | 102701865 | 102701915 |
| chr4 | 103537099 | 103537149 |
| chr4 | 112143589 | 112143639 |
| chr11 | 66194568 | 66194618 |
| chr4 | 115894994 | 115895044 |
| chr4 | 117710246 | 117710296 |
| chr4 | 130046926 | 130046976 |
| chr4 | 131804841 | 131804891 |
| chr4 | 135350975 | 135351025 |
| chr11 | 66644406 | 66644456 |
| chr4 | 140386792 | 140386842 |
| chr4 | 140426584 | 140426634 |
| chr4 | 144828129 | 144828179 |
| chr4 | 147682827 | 147682877 |
| chr4 | 157773495 | 157773545 |
| chr4 | 162201067 | 162201117 |
| chr4 | 167546643 | 167546693 |
| chr4 | 174185874 | 174185924 |
| chr4 | 176413262 | 176413312 |
| chr4 | 177200435 | 177200485 |
| chr4 | 178027984 | 178028034 |
| chr4 | 180364150 | 180364200 |
| chr4 | 180860950 | 180861000 |
| chr4 | 181278905 | 181278955 |
| chr4 | 183705996 | 183706046 |
| chr4 | 184523044 | 184523094 |
| chr4 | 184523285 | 184523335 |
| chr4 | 184523433 | 184523483 |
| chr4 | 188919134 | 188919184 |
| chr4 | 189190536 | 189190586 |
| chr4 | 191203857 | 191203907 |
| chr11 | 68557276 | 68557326 |
| chr5 | 777926 | 777976 |
| chr5 | 862246 | 862296 |
| chr5 | 1433465 | 1433515 |
| chr5 | 1847279 | 1847329 |
| chr5 | 1880981 | 1881031 |
| chr5 | 3152339 | 3152389 |
| chr5 | 6075254 | 6075304 |
| chr5 | 6075012 | 6075062 |
| chr5 | 6848267 | 6848317 |
| chr5 | 6908181 | 6908231 |
| chr5 | 9470759 | 9470809 |
| chr5 | 10345753 | 10345803 |
| chr5 | 10361782 | 10361832 |
| chr5 | 10665516 | 10665566 |
| chr5 | 12034007 | 12034057 |
| chr5 | 20815949 | 20815999 |
| chr5 | 20816003 | 20816053 |
| chr5 | 22842417 | 22842467 |
| chr5 | 23252956 | 23253006 |
| chr5 | 24018565 | 24018615 |
| chr5 | 28360930 | 28360980 |
| chr5 | 29346919 | 29346969 |
| chr5 | 29485164 | 29485214 |
| chr5 | 29973586 | 29973636 |
| chr5 | 31940504 | 31940554 |
| chr5 | 39144778 | 39144828 |
| chr5 | 50601352 | 50601402 |
| chr5 | 50884057 | 50884107 |
| chr5 | 51447251 | 51447301 |
| chr5 | 56102439 | 56102489 |
| chr11 | 70437659 | 70437709 |
| chr11 | 70548001 | 70548051 |
| chr5 | 72205071 | 72205121 |
| chr5 | 77572039 | 77572089 |
| chr5 | 78464455 | 78464505 |
| chr5 | 79733733 | 79733783 |
| chr5 | 86014941 | 86014991 |
| chr5 | 92884588 | 92884638 |
| chr11 | 71379655 | 71379705 |
| chr5 | 97303752 | 97303802 |
| chr5 | 102082978 | 102083028 |
| chr5 | 103393283 | 103393333 |
| chr5 | 116047655 | 116047705 |
| chr5 | 130011690 | 130011740 |
| chr5 | 130705553 | 130705603 |
| chr5 | 131603059 | 131603109 |
| chr5 | 133427436 | 133427486 |
| chr11 | 72795695 | 72795745 |
| chr5 | 136708348 | 136708398 |
| chr5 | 141480312 | 141480362 |
| chr5 | 145107083 | 145107133 |
| chr5 | 145619040 | 145619090 |
| chr5 | 146607337 | 146607387 |
| chr5 | 151170991 | 151171041 |
| chr5 | 162763781 | 162763831 |
| chr5 | 164347721 | 164347771 |
| chr5 | 165418224 | 165418274 |
| chr5 | 165683010 | 165683060 |
| chr5 | 171249875 | 171249925 |
| chr5 | 173231340 | 173231390 |
| chr5 | 176398135 | 176398185 |
| chr5 | 178852453 | 178852503 |
| chr5 | 179009843 | 179009893 |
| chr11 | 76481368 | 76481418 |
| chr5 | 180370282 | 180370332 |
| chr6 | 3911509 | 3911559 |
| chr6 | 4036412 | 4036462 |
| chr6 | 4756240 | 4756290 |
| chr6 | 7649893 | 7649943 |
| chr6 | 7846279 | 7846329 |
| chr6 | 7879782 | 7879832 |
| chr6 | 9071316 | 9071366 |
| chr11 | 77602056 | 77602106 |
| chr6 | 10808596 | 10808646 |
| chr6 | 11197828 | 11197878 |
| chr6 | 13885006 | 13885056 |
| chr6 | 14494024 | 14494074 |
| chr6 | 16799779 | 16799829 |
| chr6 | 17509126 | 17509176 |
| chr11 | 80295986 | 80296036 |
| chr6 | 23212789 | 23212839 |
| chr11 | 81202525 | 81202575 |
| chr11 | 81375698 | 81375748 |
| chr6 | 27880006 | 27880056 |
| chr6 | 29141233 | 29141283 |
| chr6 | 32636170 | 32636220 |
| chr6 | 33244973 | 33245023 |
| chr11 | 83858300 | 83858350 |
| chr6 | 37828214 | 37828264 |
| chr6 | 37856401 | 37856451 |
| chr6 | 38410709 | 38410759 |
| chr6 | 42481945 | 42481995 |
| chr6 | 45270899 | 45270949 |
| chr6 | 47389001 | 47389051 |
| chr6 | 66751990 | 66752040 |
| chr6 | 69185129 | 69185179 |
| chr6 | 70163184 | 70163234 |
| chr6 | 79183274 | 79183324 |
| chr6 | 80443774 | 80443824 |
| chr11 | 92627531 | 92627581 |
| chr6 | 94530234 | 94530284 |
| chr6 | 99463169 | 99463219 |
| chr6 | 109873160 | 109873210 |
| chr6 | 111893885 | 111893935 |
| chr6 | 119293784 | 119293834 |
| chr6 | 124479787 | 124479837 |
| chr6 | 125187748 | 125187798 |
| chr6 | 139492745 | 139492795 |
| chr6 | 139948162 | 139948212 |
| chr6 | 145596648 | 145596698 |
| chr6 | 147959356 | 147959406 |
| chr6 | 151842009 | 151842059 |
| chr6 | 154987666 | 154987716 |
| chr6 | 155502927 | 155502977 |
| chr6 | 158355412 | 158355462 |
| chr6 | 159076567 | 159076617 |
| chr6 | 160049792 | 160049842 |
| chr11 | 101862778 | 101862828 |
| chr6 | 160601776 | 160601826 |
| chr11 | 102056840 | 102056890 |
| chr6 | 161931711 | 161931761 |
| chr6 | 164255458 | 164255508 |
| chr11 | 102935242 | 102935292 |
| chr11 | 103269781 | 103269831 |
| chr6 | 166845192 | 166845242 |
| chr11 | 104683558 | 104683608 |
| chr6 | 167937651 | 167937701 |
| chr6 | 168441729 | 168441779 |
| chr6 | 168897615 | 168897665 |
| chr6 | 169000339 | 169000389 |
| chr6 | 169000587 | 169000637 |
| chr6 | 169000863 | 169000913 |
| chr6 | 169784417 | 169784467 |
| chr11 | 106984380 | 106984430 |
| chr7 | 1638082 | 1638132 |
| chr7 | 1638917 | 1638967 |
| chr7 | 3884934 | 3884984 |
| chr7 | 4112694 | 4112744 |
| chr7 | 4433335 | 4433385 |
| chr7 | 4732835 | 4732885 |
| chr7 | 11004196 | 11004246 |
| chr7 | 13862019 | 13862069 |
| chr7 | 14028302 | 14028352 |
| chr7 | 15356821 | 15356871 |
| chr7 | 23411080 | 23411130 |
| chr7 | 23410802 | 23410852 |
| chr7 | 23707879 | 23707929 |
| chr7 | 25888826 | 25888876 |
| chr7 | 27435310 | 27435360 |
| chr7 | 33213725 | 33213775 |
| chr7 | 43106098 | 43106148 |
| chr7 | 43332609 | 43332659 |
| chr7 | 44896111 | 44896161 |
| chr7 | 44900805 | 44900855 |
| chr7 | 45032493 | 45032543 |
| chr7 | 47398572 | 47398622 |
| chr7 | 47436861 | 47436911 |
| chr7 | 48022704 | 48022754 |
| chr7 | 49011312 | 49011362 |
| chr7 | 50834675 | 50834725 |
| chr7 | 70887096 | 70887146 |
| chr7 | 79780658 | 79780708 |
| chr7 | 99790839 | 99790889 |
| chr7 | 100395349 | 100395399 |
| chr7 | 103764666 | 103764716 |
| chr7 | 104632041 | 104632091 |
| chr7 | 106930896 | 106930946 |
| chr7 | 128018715 | 128018765 |
| chr7 | 135314001 | 135314051 |
| chr7 | 137714541 | 137714591 |
| chr7 | 138997089 | 138997139 |
| chr7 | 143645153 | 143645203 |
| chr7 | 144533638 | 144533688 |
| chr7 | 144533581 | 144533631 |
| chr7 | 146183546 | 146183596 |
| chr7 | 149612898 | 149612948 |
| chr11 | 119287375 | 119287425 |
| chr7 | 152672227 | 152672277 |
| chr7 | 153017792 | 153017842 |
| chr7 | 154231213 | 154231263 |
| chr7 | 154791724 | 154791774 |
| chr11 | 119980741 | 119980791 |
| chr7 | 157481674 | 157481724 |
| chr7 | 157520136 | 157520186 |
| chr7 | 158249517 | 158249567 |
| chr7 | 158249299 | 158249349 |
| chr8 | 1583404 | 1583454 |
| chr8 | 1979526 | 1979576 |
| chr8 | 2615962 | 2616012 |
| chr8 | 3308114 | 3308164 |
| chr8 | 3634111 | 3634161 |
| chr8 | 5240776 | 5240826 |
| chr8 | 6240425 | 6240475 |
| chr8 | 9434670 | 9434720 |
| chr8 | 10057045 | 10057095 |
| chr8 | 10056913 | 10056963 |
| chr8 | 13599853 | 13599903 |
| chr8 | 25045804 | 25045854 |
| chr11 | 123845138 | 123845188 |
| chr8 | 38650059 | 38650109 |
| chr8 | 41478100 | 41478150 |
| chr8 | 42750055 | 42750105 |
| chr8 | 49135824 | 49135874 |
| chr8 | 52366310 | 52366360 |
| chr8 | 52367752 | 52367802 |
| chr8 | 52939732 | 52939782 |
| chr8 | 53897879 | 53897929 |
| chr8 | 55955159 | 55955209 |
| chr8 | 56960287 | 56960337 |
| chr8 | 58970597 | 58970647 |
| chr11 | 125483315 | 125483365 |
| chr10 | 18737079 | 18737129 |
| chr8 | 65614653 | 65614703 |
| chr8 | 65969801 | 65969851 |
| chr8 | 70668833 | 70668883 |
| chr8 | 74353006 | 74353056 |
| chr8 | 88904237 | 88904287 |
| chr8 | 91427126 | 91427176 |
| chr8 | 92912995 | 92913045 |
| chr8 | 95784891 | 95784941 |
| chr8 | 96906702 | 96906752 |
| chr8 | 97567005 | 97567055 |
| chr8 | 100813728 | 100813778 |
| chr8 | 102481532 | 102481582 |
| chr8 | 105087841 | 105087891 |
| chr8 | 107821067 | 107821117 |
| chr8 | 109802643 | 109802693 |
| chr8 | 112577804 | 112577854 |
| chr8 | 114430185 | 114430235 |
| chr8 | 114930000 | 114930050 |
| chr8 | 117756124 | 117756174 |
| chr8 | 120092048 | 120092098 |
| chr8 | 120932954 | 120933004 |
| chr8 | 128354167 | 128354217 |
| chr8 | 130989970 | 130990020 |
| chr8 | 134719620 | 134719670 |
| chr8 | 135927902 | 135927952 |
| chr8 | 136829502 | 136829552 |
| chr8 | 142881525 | 142881575 |
| chr8 | 142990309 | 142990359 |
| chr8 | 143357322 | 143357372 |
| chr8 | 144016000 | 144016050 |
| chr8 | 144015752 | 144015802 |
| chr8 | 144732070 | 144732120 |
| chr8 | 145327350 | 145327400 |
| chr9 | 12967494 | 12967544 |
| chr9 | 15799243 | 15799293 |
| chr9 | 22853319 | 22853369 |
| chr9 | 24561548 | 24561598 |
| chr11 | 133125404 | 133125454 |
| chr9 | 43374178 | 43374228 |
| chr9 | 69765980 | 69766030 |
| chr9 | 70386210 | 70386260 |
| chr9 | 70600222 | 70600272 |
| chr9 | 77354998 | 77355048 |
| chr9 | 86017377 | 86017427 |
| chr9 | 87313580 | 87313630 |
| chr9 | 87315133 | 87315183 |
| chr9 | 89910194 | 89910244 |
| chr10 | 20824768 | 20824818 |
| chr11 | 134025879 | 134025929 |
| chr11 | 134038896 | 134038946 |
| chr11 | 134039170 | 134039220 |
| chr11 | 134039734 | 134039784 |
| chr9 | 90809618 | 90809668 |
| chr9 | 94309238 | 94309288 |
| chr9 | 98259583 | 98259633 |
| chr9 | 103295004 | 103295054 |
| chr9 | 104801964 | 104802014 |
| chr9 | 107430323 | 107430373 |
| chr9 | 110621683 | 110621733 |
| chr9 | 116859020 | 116859070 |
| chr9 | 117091728 | 117091778 |
| chr9 | 117148295 | 117148345 |
| chr9 | 121197902 | 121197952 |
| chr12 | 1393540 | 1393590 |
| chr9 | 122288132 | 122288182 |
| chr9 | 126557199 | 126557249 |
| chr9 | 127713084 | 127713134 |
| chr9 | 129403868 | 129403918 |
| chr9 | 129467695 | 129467745 |
| chr9 | 129519829 | 129519879 |
| chr9 | 133107744 | 133107794 |
| chr9 | 133171727 | 133171777 |
| chr9 | 133500141 | 133500191 |
| chr9 | 133711537 | 133711587 |
| chr9 | 134955317 | 134955367 |
| chr9 | 135800449 | 135800499 |
| chr12 | 3690294 | 3690344 |
| chr12 | 4313314 | 4313364 |
| chr12 | 7796596 | 7796646 |
| chr12 | 7797555 | 7797605 |
| chr12 | 7919404 | 7919454 |
| chr12 | 12333686 | 12333736 |
| chr12 | 12471664 | 12471714 |
| chr12 | 14332264 | 14332314 |
| chr12 | 14363928 | 14363978 |
| chr12 | 19578326 | 19578376 |
| chr12 | 21053440 | 21053490 |
| chr12 | 22799996 | 22800046 |
| chr12 | 31882106 | 31882156 |
| chr12 | 41040841 | 41040891 |
| chr12 | 43822556 | 43822606 |
| chr12 | 45596122 | 45596172 |
| chr12 | 46097142 | 46097192 |
| chr12 | 47327056 | 47327106 |
| chr12 | 50562628 | 50562678 |
| chr10 | 26904179 | 26904229 |
| chr12 | 58234923 | 58234973 |
| chr12 | 58809799 | 58809849 |
| chr12 | 73685801 | 73685851 |
| chr10 | 1205870 | 1205920 |
| chr12 | 82119256 | 82119306 |
| chr12 | 94983620 | 94983670 |
| chr12 | 95001476 | 95001526 |
| chr12 | 97508857 | 97508907 |
| chr12 | 98664696 | 98664746 |
| chr12 | 100461814 | 100461864 |
| chr12 | 101929708 | 101929758 |
| chr12 | 103145870 | 103145920 |
| chr12 | 103326196 | 103326246 |
| chr12 | 103874168 | 103874218 |
| chr12 | 105215436 | 105215486 |
| chr12 | 107378215 | 107378265 |
| chr12 | 115473906 | 115473956 |
| chr12 | 118776381 | 118776431 |
| chr12 | 119868187 | 119868237 |
| chr12 | 120116182 | 120116232 |
| chr12 | 123476318 | 123476368 |
| chr12 | 123480952 | 123481002 |
| chr12 | 123569190 | 123569240 |
| chr12 | 124245896 | 124245946 |
| chr12 | 124539849 | 124539899 |
| chr12 | 127623242 | 127623292 |
| chr12 | 128713301 | 128713351 |
| chr12 | 129761044 | 129761094 |
| chr12 | 130366071 | 130366121 |
| chr10 | 35399720 | 35399770 |
| chr13 | 18993068 | 18993118 |
| chr13 | 19476914 | 19476964 |
| chr13 | 20715923 | 20715973 |
| chr13 | 21333662 | 21333712 |
| chr13 | 22758755 | 22758805 |
| chr13 | 22758704 | 22758754 |
| chr10 | 36658059 | 36658109 |
| chr13 | 25305442 | 25305492 |
| chr13 | 27182945 | 27182995 |
| chr13 | 28623492 | 28623542 |
| chr13 | 28962663 | 28962713 |
| chr13 | 29035450 | 29035500 |
| chr13 | 30564190 | 30564240 |
| chr13 | 40377103 | 40377153 |
| chr13 | 41181593 | 41181643 |
| chr10 | 37995435 | 37995485 |
| chr10 | 37995149 | 37995199 |
| chr13 | 43459903 | 43459953 |
| chr13 | 45923400 | 45923450 |
| chr13 | 46216481 | 46216531 |
| chr13 | 49834829 | 49834879 |
| chr13 | 50371988 | 50372038 |
| chr13 | 61283907 | 61283957 |
| chr13 | 61389286 | 61389336 |
| chr13 | 64066618 | 64066668 |
| chr13 | 69134246 | 69134296 |
| chr13 | 71873223 | 71873273 |
| chr10 | 42732349 | 42732399 |
| chr13 | 77836863 | 77836913 |
| chr13 | 83848301 | 83848351 |
| chr13 | 85240987 | 85241037 |
| chr13 | 86771074 | 86771124 |
| chr13 | 91645966 | 91646016 |
| chr13 | 93019902 | 93019952 |
| chr13 | 95392261 | 95392311 |
| chr13 | 98062290 | 98062340 |
| chr13 | 104745758 | 104745808 |
| chr13 | 104805482 | 104805532 |
| chr13 | 104920898 | 104920948 |
| chr13 | 106587316 | 106587366 |
| chr13 | 106634365 | 106634415 |
| chr13 | 107448770 | 107448820 |
| chr13 | 108580440 | 108580490 |
| chr13 | 109616557 | 109616607 |
| chr10 | 44113143 | 44113193 |
| chr13 | 111622964 | 111623014 |
| chr13 | 111829903 | 111829953 |
| chr14 | 25650268 | 25650318 |
| chr14 | 34104664 | 34104714 |
| chr14 | 37654106 | 37654156 |
| chr14 | 38604806 | 38604856 |
| chr14 | 41747354 | 41747404 |
| chr14 | 43346977 | 43347027 |
| chr14 | 43347312 | 43347362 |
| chr14 | 45968465 | 45968515 |
| chr14 | 46220196 | 46220246 |
| chr14 | 55134116 | 55134166 |
| chr14 | 56597995 | 56598045 |
| chr14 | 56598159 | 56598209 |
| chr14 | 63719275 | 63719325 |
| chr10 | 51080674 | 51080724 |
| chr14 | 75534603 | 75534653 |
| chr14 | 76569795 | 76569845 |
| chr14 | 77026322 | 77026372 |
| chr14 | 78750139 | 78750189 |
| chr14 | 79275972 | 79276022 |
| chr14 | 79478772 | 79478822 |
| chr14 | 80976521 | 80976571 |
| chr14 | 82176040 | 82176090 |
| chr14 | 83710423 | 83710473 |
| chr14 | 87588979 | 87589029 |
| chr14 | 87661952 | 87662002 |
| chr14 | 88168864 | 88168914 |
| chr14 | 88949531 | 88949581 |
| chr14 | 89568308 | 89568358 |
| chr14 | 90848986 | 90849036 |
| chr14 | 90849827 | 90849877 |
| chr10 | 55300999 | 55301049 |
| chr14 | 94017472 | 94017522 |
| chr14 | 99214087 | 99214137 |
| chr14 | 102314118 | 102314168 |
| chr14 | 103742588 | 103742638 |
| chr14 | 104031130 | 104031180 |
| chr14 | 104274141 | 104274191 |
| chr15 | 18396801 | 18396851 |
| chr15 | 20432757 | 20432807 |
| chr15 | 20765506 | 20765556 |
| chr15 | 29356786 | 29356836 |
| chr15 | 29413401 | 29413451 |
| chr15 | 29663999 | 29664049 |
| chr15 | 33733022 | 33733072 |
| chr15 | 36341117 | 36341167 |
| chr15 | 36340993 | 36341043 |
| chr15 | 51424299 | 51424349 |
| chr15 | 53501071 | 53501121 |
| chr10 | 65903931 | 65903981 |
| chr15 | 59826949 | 59826999 |
| chr15 | 62068896 | 62068946 |
| chr15 | 67367632 | 67367682 |
| chr15 | 76656241 | 76656291 |
| chr15 | 79422219 | 79422269 |
| chr15 | 79872020 | 79872070 |
| chr15 | 80102059 | 80102109 |
| chr15 | 81315284 | 81315334 |
| chr15 | 81314781 | 81314831 |
| chr15 | 83293665 | 83293715 |
| chr15 | 85052003 | 85052053 |
| chr15 | 88380808 | 88380858 |
| chr15 | 91705653 | 91705703 |
| chr15 | 96990183 | 96990233 |
| chr15 | 98237763 | 98237813 |
| chr16 | 1075700 | 1075750 |
| chr16 | 1273475 | 1273525 |
| chr16 | 4044254 | 4044304 |
| chr16 | 5248174 | 5248224 |
| chr16 | 5380161 | 5380211 |
| chr16 | 5834873 | 5834923 |
| chr16 | 7512964 | 7513014 |
| chr10 | 71196312 | 71196362 |
| chr10 | 71196111 | 71196161 |
| chr16 | 9240077 | 9240127 |
| chr16 | 15589703 | 15589753 |
| chr16 | 17931782 | 17931832 |
| chr16 | 18013410 | 18013460 |
| chr16 | 18921356 | 18921406 |
| chr10 | 71624333 | 71624383 |
| chr16 | 19734793 | 19734843 |
| chr16 | 24314443 | 24314493 |
| chr10 | 72105072 | 72105122 |
| chr16 | 28208462 | 28208512 |
| chr16 | 29276055 | 29276105 |
| chr16 | 53693173 | 53693223 |
| chr16 | 55890375 | 55890425 |
| chr16 | 58597482 | 58597532 |
| chr16 | 58995414 | 58995464 |
| chr16 | 62315749 | 62315799 |
| chr16 | 63485274 | 63485324 |
| chr16 | 66497775 | 66497825 |
| chr16 | 66762598 | 66762648 |
| chr10 | 74052020 | 74052070 |
| chr16 | 73886506 | 73886556 |
| chr16 | 73886385 | 73886435 |
| chr16 | 75401787 | 75401837 |
| chr16 | 77589290 | 77589340 |
| chr16 | 80121277 | 80121327 |
| chr16 | 80121150 | 80121200 |
| chr16 | 82742980 | 82743030 |
| chr10 | 74814408 | 74814458 |
| chr10 | 74814672 | 74814722 |
| chr17 | 259399 | 259449 |
| chr17 | 1436954 | 1437004 |
| chr17 | 6279672 | 6279722 |
| chr17 | 6280400 | 6280450 |
| chr17 | 7160274 | 7160324 |
| chr17 | 9077327 | 9077377 |
| chr17 | 10116628 | 10116678 |
| chr10 | 78848227 | 78848277 |
| chr17 | 12321590 | 12321640 |
| chr17 | 15823865 | 15823915 |
| chr17 | 16021779 | 16021829 |
| chr17 | 16589602 | 16589652 |
| chr10 | 79356589 | 79356639 |
| chr17 | 18370892 | 18370942 |
| chr17 | 19016137 | 19016187 |
| chr17 | 19486515 | 19486565 |
| chr17 | 27799718 | 27799768 |
| chr17 | 32249238 | 32249288 |

Table 15DA. Repeat Category MLT1A1

| | | |
|---|---|---|
| chr17 | 42389181 | 42389231 |
| chr10 | 82047624 | 82047674 |
| chr17 | 47822557 | 47822607 |
| chr17 | 49571045 | 49571095 |
| chr17 | 55581142 | 55581192 |
| chr17 | 60775221 | 60775271 |
| chr17 | 79652646 | 79652696 |
| chr18 | 5017849 | 5017899 |
| chr18 | 7128794 | 7128844 |
| chr18 | 7341710 | 7341760 |
| chr10 | 95605602 | 95605652 |
| chr10 | 95760596 | 95760646 |
| chr18 | 38348176 | 38348226 |
| chr18 | 49828173 | 49828223 |
| chr10 | 98244194 | 98244244 |
| chr18 | 65801419 | 65801469 |
| chr18 | 68015471 | 68015521 |
| chr10 | 102054584 | 102054634 |
| chr19 | 13606603 | 13606653 |
| chr19 | 57357731 | 57357781 |
| chr1 | 3508591 | 3508641 |
| chr10 | 118119298 | 118119348 |
| chr1 | 13329594 | 13329644 |
| chr1 | 22803523 | 22803573 |
| chr10 | 6296629 | 6296679 |
| chr1 | 31822718 | 31822768 |
| chr1 | 38623270 | 38623320 |
| chr1 | 39916475 | 39916525 |
| chr1 | 69999586 | 69999636 |
| chr1 | 89491487 | 89491537 |
| chr1 | 89490800 | 89490850 |
| chr1 | 91971378 | 91971428 |
| chr1 | 93360323 | 93360373 |
| chr1 | 93360776 | 93360826 |
| chr1 | 112336971 | 112337021 |
| chr1 | 112340213 | 112340263 |
| chr10 | 126523450 | 126523500 |
| chr1 | 157979341 | 157979391 |
| chr1 | 163039703 | 163039753 |
| chr1 | 186091563 | 186091613 |
| chr1 | 201779602 | 201779652 |
| chr10 | 130653697 | 130653747 |
| chr1 | 215347907 | 215347957 |
| chr1 | 228452409 | 228452459 |
| chr1 | 231909179 | 231909229 |
| chr1 | 234181411 | 234181461 |
| chr10 | 132168598 | 132168648 |
| chr1 | 242914268 | 242914318 |
| chr20 | 17425983 | 17426033 |
| chr20 | 25214568 | 25214618 |
| chr20 | 25214677 | 25214727 |
| chr20 | 25410958 | 25411008 |
| chr20 | 38756863 | 38756913 |
| chr20 | 46202878 | 46202928 |
| chr20 | 61559507 | 61559557 |
| chr20 | 62993513 | 62993563 |
| chr21 | 22301691 | 22301741 |
| chr21 | 26208640 | 26208690 |
| chr21 | 26554072 | 26554122 |
| chr21 | 31788138 | 31788188 |
| chr21 | 44028131 | 44028181 |
| chr21 | 46221250 | 46221300 |
| chr22 | 21687007 | 21687057 |
| chr22 | 23785817 | 23785867 |
| chr22 | 32646888 | 32646938 |
| chr22 | 35078060 | 35078110 |
| chr22 | 35528365 | 35528415 |
| chr11 | 2314129 | 2314179 |
| chr11 | 2338162 | 2338212 |
| chr11 | 2338614 | 2338664 |
| chr22 | 39032518 | 39032568 |
| chr22 | 39546152 | 39546202 |
| chr22 | 46078808 | 46078858 |
| chr22 | 46080758 | 46080808 |
| chrX | 6069324 | 6069374 |
| chrX | 6070646 | 6070696 |
| chrX | 9963176 | 9963226 |
| chrX | 12186608 | 12186658 |
| chr11 | 6732140 | 6732190 |
| chr11 | 6731923 | 6731973 |
| chrX | 56378890 | 56378940 |
| chrX | 62485450 | 62485500 |
| chrX | 101121222 | 101121272 |
| chrX | 104123657 | 104123707 |
| chrX | 115685448 | 115685498 |
| chrX | 117279575 | 117279625 |
| chrX | 117534168 | 117534218 |
| chrX | 126492679 | 126492729 |
| chrX | 131904454 | 131904504 |
| chrY | 9052531 | 9052581 |
| chrY | 16330374 | 16330424 |
| chr2 | 1070590 | 1070640 |
| chr2 | 1764358 | 1764408 |
| chr2 | 15748891 | 15748941 |
| chr2 | 18031329 | 18031379 |
| chr2 | 39246423 | 39246473 |
| chr2 | 47317603 | 47317653 |
| chr2 | 54218673 | 54218723 |
| chr11 | 28484301 | 28484351 |
| chr2 | 109269484 | 109269534 |
| chr2 | 130316228 | 130316278 |
| chr2 | 150618504 | 150618554 |
| chr2 | 170593329 | 170593379 |
| chr2 | 170780631 | 170780681 |
| chr2 | 170902111 | 170902161 |
| chr11 | 40147681 | 40147731 |
| chr2 | 204114085 | 204114135 |
| chr2 | 208729015 | 208729065 |
| chr2 | 232029091 | 232029141 |
| chr2 | 236256659 | 236256709 |
| chr2 | 238236795 | 238236845 |
| chr11 | 45717457 | 45717507 |
| chr2 | 239996012 | 239996062 |
| chr3 | 11757313 | 11757363 |
| chr3 | 23152294 | 23152344 |
| chr11 | 48058072 | 48058122 |
| chr3 | 52125602 | 52125652 |
| chr3 | 95573782 | 95573832 |
| chr3 | 105270010 | 105270060 |
| chr3 | 110388685 | 110388735 |
| chr3 | 117717428 | 117717478 |
| chr3 | 121452504 | 121452554 |
| chr3 | 129743515 | 129743565 |
| chr3 | 139058402 | 139058452 |
| chr3 | 142349181 | 142349231 |
| chr3 | 143039830 | 143039880 |
| chr3 | 158351515 | 158351565 |
| chr3 | 186107482 | 186107532 |
| chr11 | 63471638 | 63471688 |
| chr3 | 198334327 | 198334377 |
| chr4 | 1609924 | 1609974 |
| chr11 | 64700890 | 64700940 |
| chr11 | 65218662 | 65218712 |
| chr4 | 63395615 | 63395665 |
| chr4 | 63915993 | 63916043 |
| chr4 | 67429475 | 67429525 |
| chr4 | 67552484 | 67552534 |
| chr4 | 69163433 | 69163483 |
| chr4 | 100340653 | 100340703 |
| chr4 | 112504259 | 112504309 |
| chr4 | 114860281 | 114860331 |
| chr4 | 130006317 | 130006367 |
| chr4 | 130092495 | 130092545 |
| chr4 | 131684430 | 131684480 |
| chr4 | 140395544 | 140395594 |
| chr4 | 148125040 | 148125090 |
| chr4 | 149194409 | 149194459 |
| chr4 | 154068479 | 154068529 |
| chr4 | 155316431 | 155316481 |
| chr4 | 155316301 | 155316351 |
| chr4 | 170865875 | 170865925 |
| chr4 | 177549385 | 177549435 |
| chr4 | 183424077 | 183424127 |
| chr4 | 186989358 | 186989408 |
| chr4 | 188156277 | 188156327 |
| chr4 | 188156435 | 188156485 |
| chr4 | 189675471 | 189675521 |
| chr5 | 1147906 | 1147956 |
| chr5 | 10912750 | 10912800 |
| chr5 | 12239006 | 12239056 |
| chr5 | 18465138 | 18465188 |
| chr5 | 18746772 | 18746822 |
| chr5 | 18776817 | 18776867 |
| chr5 | 20739594 | 20739644 |
| chr5 | 26248755 | 26248805 |
| chr5 | 28233868 | 28233918 |
| chr5 | 78139794 | 78139844 |
| chr5 | 99282775 | 99282825 |
| chr5 | 108808701 | 108808751 |
| chr5 | 124793979 | 124794029 |
| chr5 | 145927213 | 145927263 |
| chr5 | 164922120 | 164922170 |
| chr5 | 171733637 | 171733687 |
| chr5 | 177988629 | 177988679 |
| chr6 | 3441172 | 3441222 |
| chr6 | 7267792 | 7267842 |
| chr6 | 9290298 | 9290348 |
| chr6 | 13320864 | 13320914 |
| chr6 | 14176842 | 14176892 |
| chr6 | 39084428 | 39084478 |
| chr6 | 39843570 | 39843620 |
| chr6 | 56219731 | 56219781 |
| chr6 | 83708015 | 83708065 |
| chr6 | 124725475 | 124725525 |
| chr6 | 131255497 | 131255547 |
| chr6 | 132642644 | 132642694 |
| chr6 | 158194965 | 158195015 |
| chr6 | 168145501 | 168145551 |
| chr6 | 168145801 | 168145851 |
| chr6 | 168352323 | 168352373 |
| chr6 | 168352795 | 168352845 |
| chr6 | 170650606 | 170650656 |
| chr7 | 29451979 | 29452029 |
| chr7 | 32525446 | 32525496 |
| chr7 | 43738532 | 43738582 |
| chr7 | 50677319 | 50677369 |
| chr7 | 72357940 | 72357990 |
| chr7 | 124636855 | 124636905 |
| chr10 | 17612669 | 17612719 |
| chr11 | 118845396 | 118845446 |
| chr7 | 150543396 | 150543446 |
| chr7 | 158043621 | 158043671 |
| chr7 | 158044757 | 158044807 |
| chr10 | 17966490 | 17966540 |
| chr8 | 1719367 | 1719417 |
| chr8 | 1719962 | 1720012 |
| chr10 | 18213400 | 18213450 |
| chr8 | 2767365 | 2767415 |
| chr8 | 11303063 | 11303113 |
| chr8 | 23489096 | 23489146 |
| chr8 | 24904119 | 24904169 |
| chr8 | 30185916 | 30185966 |
| chr8 | 39182303 | 39182353 |
| chr8 | 42159046 | 42159096 |
| chr8 | 55474525 | 55474575 |
| chr8 | 67966299 | 67966349 |
| chr8 | 74178911 | 74178961 |
| chr8 | 83188834 | 83188884 |
| chr11 | 128348352 | 128348402 |
| chr8 | 120118514 | 120118564 |
| chr8 | 127699054 | 127699104 |
| chr8 | 131776504 | 131776554 |
| chr8 | 141098143 | 141098193 |
| chr8 | 142939718 | 142939768 |
| chr8 | 144384107 | 144384157 |
| chr8 | 145861459 | 145861509 |
| chr9 | 6684291 | 6684341 |
| chr9 | 6904458 | 6904508 |
| chr9 | 7118221 | 7118271 |
| chr9 | 7118120 | 7118170 |
| chr9 | 16390427 | 16390477 |
| chr9 | 33687572 | 33687622 |
| chr9 | 33687318 | 33687368 |
| chr9 | 70493932 | 70493982 |
| chr9 | 78819849 | 78819899 |
| chr9 | 87390311 | 87390361 |
| chr9 | 92223237 | 92223287 |
| chr9 | 93488821 | 93488871 |
| chr9 | 96417384 | 96417434 |
| chr9 | 110866344 | 110866394 |
| chr9 | 110867319 | 110867369 |
| chr12 | 1537752 | 1537802 |
| chr12 | 1538352 | 1538402 |
| chr9 | 127937322 | 127937372 |
| chr9 | 130641227 | 130641277 |
| chr10 | 22196439 | 22196489 |
| chr12 | 16166769 | 16166819 |
| chr12 | 32267328 | 32267378 |
| chr12 | 42135594 | 42135644 |
| chr12 | 47317207 | 47317257 |
| chr10 | 27192759 | 27192809 |
| chr10 | 28812081 | 28812131 |
| chr10 | 29300672 | 29300722 |
| chr12 | 90875344 | 90875394 |
| chr12 | 92519973 | 92520023 |
| chr12 | 92942700 | 92942750 |
| chr12 | 94972803 | 94972853 |
| chr12 | 100317739 | 100317789 |
| chr12 | 120443279 | 120443329 |
| chr12 | 131786851 | 131786901 |
| chr12 | 131999569 | 131999619 |
| chr13 | 19148845 | 19148895 |
| chr13 | 19482651 | 19482701 |
| chr13 | 22480241 | 22480291 |
| chr13 | 23199912 | 23199962 |
| chr13 | 25865336 | 25865386 |
| chr13 | 38728265 | 38728315 |
| chr13 | 49862648 | 49862698 |
| chr13 | 53707291 | 53707341 |
| chr10 | 42366038 | 42366088 |
| chr13 | 62274430 | 62274480 |
| chr13 | 78655728 | 78655778 |
| chr13 | 85532201 | 85532251 |
| chr13 | 97969087 | 97969137 |
| chr10 | 45227712 | 45227762 |
| chr14 | 21708164 | 21708214 |
| chr14 | 21916565 | 21916615 |
| chr14 | 22116007 | 22116057 |
| chr14 | 25625906 | 25625956 |
| chr14 | 27133030 | 27133080 |
| chr14 | 49928480 | 49928530 |
| chr14 | 63901595 | 63901645 |
| chr14 | 69426430 | 69426480 |
| chr14 | 69600075 | 69600125 |
| chr14 | 69599905 | 69599955 |
| chr14 | 73939807 | 73939857 |
| chr14 | 80544929 | 80544979 |
| chr14 | 82569731 | 82569781 |
| chr14 | 87363378 | 87363428 |
| chr14 | 87748580 | 87748630 |
| chr14 | 91044551 | 91044601 |
| chr14 | 92327056 | 92327106 |
| chr14 | 100396278 | 100396328 |
| chr14 | 101031609 | 101031659 |
| chr14 | 103308182 | 103308232 |
| chr14 | 103973354 | 103973404 |
| chr15 | 22700248 | 22700298 |
| chr15 | 42399105 | 42399155 |
| chr15 | 52039484 | 52039534 |
| chr15 | 53600647 | 53600697 |
| chr15 | 80119673 | 80119723 |
| chr15 | 84306899 | 84306949 |
| chr16 | 6084208 | 6084258 |
| chr16 | 9082351 | 9082401 |
| chr16 | 22903640 | 22903690 |
| chr16 | 28314359 | 28314409 |
| chr16 | 48167565 | 48167615 |
| chr16 | 48167453 | 48167503 |
| chr16 | 52807808 | 52807858 |
| chr16 | 56523367 | 56523417 |
| chr16 | 58244086 | 58244136 |
| chr16 | 75279095 | 75279145 |
| chr16 | 75333135 | 75333185 |
| chr16 | 77291255 | 77291305 |
| chr16 | 89155353 | 89155403 |
| chr16 | 89155217 | 89155267 |
| chr10 | 75790824 | 75790874 |
| chr10 | 75790960 | 75791010 |
| chr17 | 8456399 | 8456449 |
| chr17 | 10132106 | 10132156 |
| chr10 | 79362760 | 79362810 |

Table 15DB. Repeat Category MLT1D-int

| | | |
|---|---|---|
| chr19 | 3685474 | 3685524 |
| chr1 | 24722027 | 24722077 |
| chr1 | 56328589 | 56328639 |
| chr1 | 158518464 | 158518514 |
| chr1 | 158520497 | 158520547 |
| chr1 | 230795152 | 230795202 |
| chr21 | 23355943 | 23355993 |
| chr21 | 44649714 | 44649764 |
| chr21 | 44650783 | 44650833 |
| chr21 | 44651638 | 44651688 |
| chr11 | 1454013 | 1454063 |
| chr11 | 1455289 | 1455339 |
| chr22 | 22680954 | 22681004 |
| chr22 | 39258564 | 39258614 |
| chr22 | 39261089 | 39261139 |
| chrX | 50505235 | 50505285 |
| chrX | 104987939 | 104987989 |
| chr2 | 57317578 | 57317628 |
| chr11 | 28788279 | 28788329 |
| chr2 | 123209273 | 123209323 |
| chr2 | 135078099 | 135078149 |
| chr2 | 135079567 | 135079617 |
| chr2 | 141639929 | 141639979 |
| chr2 | 231439665 | 231439715 |
| chr3 | 138239288 | 138239338 |
| chr4 | 30994830 | 30994880 |
| chr11 | 70003455 | 70003505 |
| chr6 | 66671001 | 66671051 |
| chr7 | 6002660 | 6002710 |
| chr7 | 108487440 | 108487490 |
| chr7 | 128762156 | 128762206 |
| chr8 | 42208643 | 42208693 |
| chr8 | 144772578 | 144772628 |
| chr8 | 145142425 | 145142475 |
| chr9 | 100455867 | 100455917 |
| chr12 | 127724608 | 127724658 |
| chr13 | 64467757 | 64467807 |
| chr16 | 59565312 | 59565362 |

Table 15DC. Repeat Category MLT1E

| | | |
|---|---|---|
| chr17 | 47831363 | 47831413 |
| chr17 | 47833546 | 47833596 |
| chr17 | 55740556 | 55740606 |
| chr17 | 59393504 | 59393554 |
| chr17 | 64715790 | 64715840 |
| chr17 | 65536725 | 65536775 |
| chr17 | 67187772 | 67187822 |
| chr17 | 68490863 | 68490913 |
| chr17 | 75657698 | 75657748 |
| chr17 | 76579015 | 76579065 |
| chr17 | 78800988 | 78801038 |
| chr17 | 79642713 | 79642763 |
| chr18 | 3109833 | 3109883 |
| chr18 | 8433646 | 8433696 |
| chr18 | 12966642 | 12966692 |
| chr18 | 18698217 | 18698267 |
| chr18 | 30045015 | 30045065 |
| chr18 | 30607603 | 30607653 |
| chr18 | 31622376 | 31622426 |
| chr18 | 32443928 | 32443978 |
| chr18 | 32655270 | 32655320 |
| chr18 | 32871641 | 32871691 |
| chr18 | 32894687 | 32894737 |
| chr18 | 34074086 | 34074136 |
| chr10 | 5013846 | 5013896 |
| chr18 | 36514156 | 36514206 |
| chr18 | 41972856 | 41972906 |
| chr18 | 42353991 | 42354041 |
| chr18 | 46584992 | 46585042 |
| chr18 | 49525078 | 49525128 |
| chr18 | 50021958 | 50022008 |
| chr18 | 52856287 | 52856337 |
| chr18 | 56276892 | 56276942 |
| chr18 | 56810555 | 56810605 |
| chr18 | 58496275 | 58496325 |
| chr18 | 63077178 | 63077228 |
| chr18 | 65396154 | 65396204 |
| chr18 | 66710209 | 66710259 |
| chr18 | 73517328 | 73517378 |
| chr18 | 74265236 | 74265286 |
| chr19 | 329657 | 329707 |
| chr19 | 1118055 | 1118105 |
| chr19 | 1290155 | 1290205 |
| chr19 | 1691821 | 1691871 |
| chr19 | 2278146 | 2278196 |
| chr19 | 3502015 | 3502065 |
| chr19 | 4290134 | 4290184 |
| chr19 | 4290360 | 4290410 |
| chr19 | 4618040 | 4618090 |
| chr19 | 6594897 | 6594947 |
| chr19 | 9418830 | 9418880 |
| chr19 | 13191417 | 13191467 |
| chr19 | 14260698 | 14260748 |
| chr19 | 19578424 | 19578474 |
| chr19 | 21557352 | 21557402 |
| chr19 | 21556880 | 21556930 |
| chr19 | 23187662 | 23187712 |
| chr19 | 37671585 | 37671635 |
| chr19 | 40156262 | 40156312 |
| chr10 | 105862554 | 105862604 |
| chr19 | 47698558 | 47698608 |
| chr19 | 49750219 | 49750269 |
| chr19 | 59148441 | 59148491 |
| chr19 | 59678885 | 59678935 |
| chr19 | 60673203 | 60673253 |
| chr19 | 60674467 | 60674517 |
| chr19 | 60990494 | 60990544 |
| chr19 | 63361953 | 63362003 |
| chr19 | 63690504 | 63690554 |
| chr1 | 3716912 | 3716962 |
| chr1 | 3825639 | 3825689 |
| chr1 | 7883054 | 7883104 |
| chr1 | 9000468 | 9000518 |
| chr1 | 13148248 | 13148298 |
| chr1 | 23841184 | 23841234 |
| chr1 | 26757461 | 26757511 |
| chr1 | 27010871 | 27010921 |
| chr1 | 37686990 | 37687040 |
| chr1 | 39494845 | 39494895 |
| chr1 | 41138946 | 41138996 |
| chr1 | 59489448 | 59489498 |
| chr1 | 63234548 | 63234598 |
| chr1 | 64594611 | 64594661 |
| chr1 | 70284792 | 70284842 |
| chr1 | 73172795 | 73172845 |
| chr1 | 85407219 | 85407269 |
| chr1 | 85407141 | 85407191 |
| chr10 | 6684743 | 6684793 |
| chr1 | 94913420 | 94913470 |
| chr1 | 94913735 | 94913785 |
| chr1 | 94914117 | 94914167 |
| chr1 | 105674740 | 105674790 |
| chr1 | 119421025 | 119421075 |
| chr1 | 119420802 | 119420852 |
| chr1 | 144829958 | 144830008 |
| chr1 | 157982695 | 157982745 |
| chr1 | 160580408 | 160580458 |
| chr1 | 164685897 | 164685947 |
| chr1 | 165297962 | 165298012 |
| chr1 | 166644595 | 166644645 |
| chr1 | 172765596 | 172765646 |
| chr1 | 172765343 | 172765393 |
| chr10 | 129675692 | 129675742 |
| chr1 | 189103439 | 189103489 |
| chr1 | 191914983 | 191915033 |
| chr1 | 195544555 | 195544605 |
| chr1 | 216740268 | 216740318 |
| chr1 | 223197391 | 223197441 |
| chr1 | 223705206 | 223705256 |
| chr1 | 225239001 | 225239051 |
| chr1 | 225238803 | 225238853 |
| chr1 | 233176681 | 233176731 |
| chr10 | 131636869 | 131636919 |
| chr1 | 241770014 | 241770064 |
| chr1 | 243813655 | 243813705 |
| chr1 | 244547850 | 244547900 |
| chr20 | 669783 | 669833 |
| chr20 | 3043556 | 3043606 |
| chr20 | 5642968 | 5643018 |
| chr20 | 9185330 | 9185380 |
| chr20 | 17168886 | 17168936 |
| chr20 | 17836425 | 17836475 |
| chr20 | 17836216 | 17836266 |
| chr20 | 20259800 | 20259850 |
| chr20 | 30766345 | 30766395 |
| chr10 | 133469033 | 133469083 |
| chr10 | 133468972 | 133469022 |
| chr10 | 133841192 | 133841242 |
| chr20 | 44125679 | 44125729 |
| chr20 | 50616436 | 50616486 |
| chr20 | 53230999 | 53231049 |
| chr20 | 53919098 | 53919148 |
| chr20 | 54221081 | 54221131 |
| chr20 | 55005483 | 55005533 |
| chr20 | 55801676 | 55801726 |
| chr20 | 56353794 | 56353844 |
| chr20 | 56578425 | 56578475 |
| chr20 | 58396502 | 58396552 |
| chr20 | 59910057 | 59910107 |
| chr20 | 62140316 | 62140366 |
| chr20 | 62265276 | 62265326 |
| chr20 | 62863613 | 62863663 |
| chr21 | 10087446 | 10087496 |
| chr21 | 18272127 | 18272177 |
| chr21 | 18422182 | 18422232 |
| chr21 | 22562168 | 22562218 |
| chr21 | 31130187 | 31130237 |
| chr21 | 31324199 | 31324249 |
| chr21 | 31324811 | 31324861 |
| chr21 | 31746148 | 31746198 |
| chr21 | 33640408 | 33640458 |
| chr21 | 34802071 | 34802121 |
| chr21 | 34802604 | 34802654 |
| chr21 | 37571180 | 37571230 |
| chr21 | 44430106 | 44430156 |
| chr21 | 44440619 | 44440669 |
| chr21 | 44454007 | 44454057 |
| chr21 | 44454910 | 44454960 |
| chr21 | 44454737 | 44454787 |
| chr21 | 45073476 | 45073526 |
| chr21 | 45409810 | 45409860 |
| chr21 | 45525063 | 45525113 |
| chr21 | 46221250 | 46221300 |
| chr21 | 46222157 | 46222207 |
| chr21 | 46286537 | 46286587 |
| chr21 | 46357185 | 46357235 |
| chr21 | 46472555 | 46472605 |
| chr21 | 46473125 | 46473175 |
| chr22 | 18147335 | 18147385 |
| chr22 | 19189270 | 19189320 |
| chr22 | 28317612 | 28317662 |
| chr22 | 29098859 | 29098909 |
| chr22 | 31668958 | 31669008 |
| chr22 | 31823553 | 31823603 |
| chr22 | 32137870 | 32137920 |
| chr22 | 34097825 | 34097875 |
| chr22 | 35154982 | 35155032 |
| chr22 | 43732857 | 43732907 |
| chr22 | 47854320 | 47854370 |
| chr22 | 48160123 | 48160173 |
| chr22 | 48182691 | 48182741 |
| chrX | 6809179 | 6809229 |
| chrX | 6809112 | 6809162 |
| chrX | 13363565 | 13363615 |
| chrX | 14620027 | 14620077 |
| chrX | 14739785 | 14739835 |
| chrX | 21791067 | 21791117 |
| chrX | 23199210 | 23199260 |
| chrX | 23501868 | 23501918 |
| chrX | 26004641 | 26004691 |
| chrX | 35056983 | 35057033 |
| chrX | 35290601 | 35290651 |
| chr11 | 6732140 | 6732190 |
| chrX | 43494342 | 43494392 |
| chrX | 43696223 | 43696273 |
| chrX | 44964935 | 44964985 |
| chrX | 49663992 | 49664042 |
| chrX | 55961522 | 55961572 |
| chrX | 64062729 | 64062779 |
| chrX | 72004917 | 72004967 |
| chrX | 86355721 | 86355771 |
| chrX | 86946042 | 86946092 |
| chrX | 92270844 | 92270894 |
| chrX | 94608237 | 94608287 |
| chrX | 105149088 | 105149138 |
| chrX | 110026541 | 110026591 |
| chr11 | 10556227 | 10556277 |
| chr11 | 10556684 | 10556734 |
| chr11 | 10556750 | 10556800 |
| chrX | 128774648 | 128774698 |
| chrX | 130089934 | 130089984 |
| chrX | 130408767 | 130408817 |
| chrX | 140290782 | 140290832 |
| chrX | 144308906 | 144308956 |
| chrX | 152949555 | 152949605 |
| chrY | 4552353 | 4552403 |
| chrY | 15599217 | 15599267 |
| chr2 | 2022813 | 2022863 |
| chr2 | 2832036 | 2832086 |
| chr2 | 4816808 | 4816858 |
| chr2 | 4816632 | 4816682 |
| chr2 | 5543347 | 5543397 |
| chr11 | 17678055 | 17678105 |
| chr2 | 19225058 | 19225108 |
| chr2 | 19520116 | 19520166 |
| chr2 | 20895353 | 20895403 |
| chr2 | 23326847 | 23326897 |
| chr10 | 11814038 | 11814088 |
| chr2 | 81902922 | 81902972 |
| chr11 | 31030017 | 31030067 |
| chr2 | 121160429 | 121160479 |
| chr2 | 122247202 | 122247252 |
| chr2 | 132882192 | 132882242 |
| chr2 | 146157555 | 146157605 |
| chr2 | 147798908 | 147798958 |
| chr2 | 166993330 | 166993380 |
| chr11 | 35907226 | 35907276 |
| chr2 | 170762171 | 170762221 |
| chr2 | 171933491 | 171933541 |
| chr2 | 191819513 | 191819563 |
| chr2 | 191852250 | 191852300 |
| chr2 | 195726434 | 195726484 |
| chr2 | 196582938 | 196582988 |
| chr2 | 196584147 | 196584197 |
| chr11 | 39333875 | 39333925 |
| chr2 | 202628331 | 202628381 |
| chr2 | 208508603 | 208508653 |
| chr11 | 42117643 | 42117693 |
| chr11 | 42337461 | 42337511 |
| chr2 | 225166052 | 225166102 |
| chr2 | 226019697 | 226019747 |
| chr2 | 229535737 | 229535787 |
| chr2 | 229621126 | 229621176 |
| chr2 | 236301651 | 236301701 |
| chr2 | 239833185 | 239833235 |
| chr2 | 240546021 | 240546071 |
| chr2 | 240896499 | 240896549 |
| chr11 | 46137660 | 46137710 |
| chr3 | 3685495 | 3685545 |
| chr3 | 21627246 | 21627296 |
| chr3 | 21700082 | 21700132 |
| chr3 | 22197795 | 22197845 |
| chr3 | 23722812 | 23722862 |
| chr3 | 37166996 | 37167046 |
| chr11 | 47871256 | 47871306 |
| chr3 | 42412715 | 42412765 |
| chr3 | 42412465 | 42412515 |
| chr3 | 42498317 | 42498367 |
| chr3 | 46758851 | 46758901 |
| chr3 | 59382295 | 59382345 |
| chr3 | 59382192 | 59382242 |
| chr11 | 57172786 | 57172836 |
| chr3 | 66582899 | 66582949 |
| chr3 | 77739751 | 77739801 |
| chr3 | 81846521 | 81846571 |
| chr3 | 86881111 | 86881161 |
| chr3 | 95632183 | 95632233 |
| chr3 | 102850422 | 102850472 |
| chr3 | 102908967 | 102909017 |
| chr3 | 127171332 | 127171382 |
| chr3 | 130057005 | 130057055 |
| chr3 | 131233285 | 131233335 |
| chr3 | 132690194 | 132690244 |
| chr3 | 137705213 | 137705263 |
| chr3 | 142968468 | 142968518 |
| chr3 | 142968378 | 142968428 |
| chr3 | 152952931 | 152952981 |
| chr3 | 156279560 | 156279610 |
| chr3 | 156279468 | 156279518 |
| chr3 | 164930849 | 164930899 |
| chr3 | 172320890 | 172320940 |
| chr3 | 175749358 | 175749408 |
| chr3 | 178311695 | 178311745 |
| chr3 | 195873836 | 195873886 |
| chr11 | 63525125 | 63525175 |
| chr4 | 2249702 | 2249752 |
| chr4 | 3775205 | 3775255 |
| chr4 | 4078325 | 4078375 |
| chr4 | 4204483 | 4204533 |
| chr4 | 9255641 | 9255691 |
| chr4 | 16434193 | 16434243 |
| chr4 | 16496468 | 16496518 |
| chr4 | 17743014 | 17743064 |
| chr4 | 21937470 | 21937520 |
| chr4 | 21982637 | 21982687 |
| chr4 | 22497116 | 22497166 |
| chr4 | 26770351 | 26770401 |
| chr4 | 26770292 | 26770342 |
| chr4 | 33558999 | 33559049 |
| chr4 | 35295748 | 35295798 |
| chr4 | 38273593 | 38273643 |
| chr4 | 39929681 | 39929731 |
| chr4 | 55108506 | 55108556 |
| chr4 | 68344529 | 68344579 |
| chr4 | 78363184 | 78363234 |
| chr4 | 81743745 | 81743795 |
| chr4 | 92045141 | 92045191 |
| chr4 | 96028975 | 96029025 |
| chr4 | 96780225 | 96780275 |
| chr4 | 131700058 | 131700108 |
| chr4 | 132974327 | 132974377 |
| chr4 | 142706444 | 142706494 |
| chr4 | 142706241 | 142706291 |
| chr4 | 160937083 | 160937133 |
| chr4 | 160937262 | 160937312 |
| chr4 | 163844864 | 163844914 |
| chr4 | 168460292 | 168460342 |
| chr4 | 168460405 | 168460455 |
| chr4 | 168460636 | 168460686 |
| chr4 | 168460856 | 168460906 |
| chr4 | 169125144 | 169125194 |
| chr4 | 171177177 | 171177227 |
| chr4 | 174542348 | 174542398 |
| chr4 | 177807252 | 177807302 |
| chr4 | 180740683 | 180740733 |
| chr4 | 184837525 | 184837575 |
| chr11 | 67969001 | 67969051 |
| chr4 | 186207917 | 186207967 |
| chr4 | 186209986 | 186210036 |
| chr4 | 186210138 | 186210188 |
| chr4 | 189498010 | 189498060 |
| chr4 | 189851035 | 189851085 |
| chr11 | 68590792 | 68590842 |
| chr5 | 1436087 | 1436137 |
| chr5 | 8346930 | 8346980 |
| chr5 | 15920994 | 15921044 |
| chr5 | 16866919 | 16866969 |
| chr5 | 17550893 | 17550943 |
| chr5 | 17674466 | 17674516 |
| chr5 | 17698190 | 17698240 |
| chr5 | 20650808 | 20650858 |
| chr5 | 21188635 | 21188685 |
| chr5 | 22161951 | 22162001 |
| chr5 | 23370499 | 23370549 |
| chr5 | 33279928 | 33279978 |
| chr5 | 36834733 | 36834783 |
| chr5 | 37426668 | 37426718 |
| chr5 | 44995165 | 44995215 |
| chr5 | 49777809 | 49777859 |
| chr5 | 58937478 | 58937528 |
| chr5 | 68149252 | 68149302 |
| chr5 | 68591236 | 68591286 |
| chr5 | 70768204 | 70768254 |
| chr5 | 74281084 | 74281134 |
| chr5 | 78280975 | 78281025 |
| chr5 | 83930019 | 83930069 |
| chr5 | 84359062 | 84359112 |
| chr5 | 96223920 | 96223970 |
| chr5 | 96223831 | 96223881 |
| chr5 | 97894081 | 97894131 |
| chr5 | 99659307 | 99659357 |
| chr5 | 99876197 | 99876247 |
| chr5 | 108095800 | 108095850 |
| chr5 | 110087810 | 110087860 |
| chr5 | 114656062 | 114656112 |
| chr5 | 117365367 | 117365417 |
| chr5 | 123354510 | 123354560 |
| chr5 | 128976009 | 128976059 |
| chr10 | 14612842 | 14612892 |
| chr11 | 73994890 | 73994940 |
| chr10 | 14656032 | 14656082 |
| chr5 | 172417971 | 172418021 |
| chr5 | 176817836 | 176817886 |
| chr5 | 176818537 | 176818587 |
| chr5 | 177703831 | 177703881 |
| chr5 | 178017199 | 178017249 |
| chr5 | 179728503 | 179728553 |
| chr5 | 179739647 | 179739697 |
| chr11 | 76750950 | 76751000 |
| chr11 | 76755216 | 76755266 |
| chr6 | 2302240 | 2302290 |
| chr11 | 76916593 | 76916643 |
| chr6 | 3120230 | 3120280 |
| chr6 | 5922901 | 5922951 |
| chr6 | 6226915 | 6226965 |
| chr6 | 8393893 | 8393943 |
| chr6 | 10279733 | 10279783 |
| chr6 | 15107176 | 15107226 |
| chr6 | 15329248 | 15329298 |
| chr6 | 18402408 | 18402458 |
| chr11 | 80427901 | 80427951 |
| chr6 | 30013905 | 30013955 |
| chr6 | 48613004 | 48613054 |
| chr6 | 54297963 | 54298013 |
| chr6 | 65357455 | 65357505 |
| chr6 | 66948363 | 66948413 |
| chr6 | 84668687 | 84668737 |
| chr6 | 84668443 | 84668493 |
| chr6 | 94439856 | 94439906 |
| chr6 | 98763579 | 98763629 |
| chr6 | 103294987 | 103295037 |
| chr6 | 103462261 | 103462311 |
| chr6 | 107130051 | 107130101 |
| chr6 | 110893963 | 110894013 |
| chr6 | 113480283 | 113480333 |
| chr6 | 114904747 | 114904797 |
| chr6 | 115232969 | 115233019 |
| chr6 | 123177975 | 123178025 |
| chr6 | 130973613 | 130973663 |
| chr6 | 140543421 | 140543471 |
| chr6 | 146472859 | 146472909 |
| chr6 | 146472598 | 146472648 |
| chr6 | 150935692 | 150935742 |
| chr6 | 157931284 | 157931334 |
| chr6 | 162618483 | 162618533 |
| chr6 | 162618970 | 162619020 |
| chr6 | 162618710 | 162618760 |
| chr6 | 163974095 | 163974145 |
| chr6 | 163974177 | 163974227 |
| chr6 | 164696499 | 164696549 |
| chr6 | 165343844 | 165343894 |
| chr6 | 165883566 | 165883616 |
| chr6 | 166580567 | 166580617 |
| chr6 | 166581842 | 166581892 |
| chr11 | 105567462 | 105567512 |
| chr7 | 227093 | 227143 |
| chr7 | 2009656 | 2009706 |
| chr7 | 2541050 | 2541100 |
| chr7 | 2542692 | 2542742 |
| chr7 | 2602788 | 2602838 |
| chr7 | 3739170 | 3739220 |
| chr11 | 108553805 | 108553855 |
| chr7 | 4598522 | 4598572 |
| chr7 | 4921226 | 4921276 |
| chr7 | 7365966 | 7366016 |
| chr7 | 24478207 | 24478257 |
| chr7 | 35194389 | 35194439 |
| chr7 | 44999757 | 44999807 |
| chr7 | 46766467 | 46766517 |
| chr7 | 48099671 | 48099721 |
| chr7 | 49297205 | 49297255 |
| chr7 | 49886350 | 49886400 |
| chr7 | 51711497 | 51711547 |
| chr7 | 56959364 | 56959414 |
| chr7 | 63174518 | 63174568 |
| chr7 | 63378302 | 63378352 |
| chr7 | 67727538 | 67727588 |
| chr7 | 68988411 | 68988461 |
| chr7 | 81776591 | 81776641 |
| chr7 | 90277525 | 90277575 |
| chr7 | 90698367 | 90698417 |
| chr7 | 109179048 | 109179098 |
| chr7 | 117833906 | 117833956 |
| chr7 | 135550670 | 135550720 |
| chr7 | 140941959 | 140942009 |
| chr7 | 145016385 | 145016435 |
| chr11 | 119180074 | 119180124 |
| chr7 | 152109282 | 152109332 |
| chr7 | 158367986 | 158368036 |
| chr8 | 600387 | 600437 |
| chr8 | 600706 | 600756 |
| chr8 | 2918923 | 2918973 |
| chr8 | 4040892 | 4040942 |
| chr8 | 4616838 | 4616888 |
| chr8 | 5452171 | 5452221 |
| chr11 | 124059447 | 124059497 |
| chr8 | 32451750 | 32451800 |
| chr8 | 34602931 | 34602981 |
| chr8 | 41363422 | 41363472 |
| chr8 | 61489135 | 61489185 |
| chr11 | 125514648 | 125514698 |
| chr11 | 125819610 | 125819660 |
| chr8 | 80353864 | 80353914 |
| chr8 | 84923672 | 84923722 |
| chr8 | 91391603 | 91391653 |
| chr8 | 104758130 | 104758180 |
| chr11 | 128313590 | 128313640 |
| chr8 | 107914805 | 107914855 |
| chr8 | 109899259 | 109899309 |
| chr8 | 112720251 | 112720301 |
| chr8 | 114627581 | 114627631 |
| chr8 | 114775405 | 114775455 |
| chr8 | 117372070 | 117372120 |
| chr8 | 118485921 | 118485971 |
| chr10 | 19196079 | 19196129 |
| chr8 | 135728319 | 135728369 |
| chr8 | 139902855 | 139902905 |
| chr8 | 142592164 | 142592214 |
| chr8 | 144314346 | 144314396 |
| chr8 | 144621426 | 144621476 |
| chr8 | 144943066 | 144943116 |
| chr9 | 2738496 | 2738546 |
| chr9 | 15083012 | 15083062 |
| chr11 | 132392491 | 132392541 |
| chr11 | 132392372 | 132392422 |
| chr9 | 15552274 | 15552324 |
| chr9 | 23469176 | 23469226 |
| chr11 | 132823075 | 132823125 |
| chr9 | 27622227 | 27622277 |
| chr9 | 30080616 | 30080666 |
| chr11 | 133203912 | 133203962 |
| chr9 | 34669728 | 34669778 |
| chr9 | 38473512 | 38473562 |
| chr9 | 73253139 | 73253189 |
| chr9 | 76436141 | 76436191 |
| chr9 | 81140121 | 81140171 |
| chr9 | 82198898 | 82198948 |
| chr9 | 92687906 | 92687956 |
| chr9 | 98261589 | 98261639 |
| chr9 | 103455573 | 103455623 |
| chr9 | 106050739 | 106050789 |
| chr9 | 119808920 | 119808970 |
| chr9 | 130941445 | 130941495 |
| chr12 | 2755981 | 2756031 |
| chr9 | 132812854 | 132812904 |
| chr9 | 134060962 | 134061012 |
| chr9 | 134280729 | 134280779 |
| chr9 | 135706488 | 135706538 |
| chr10 | 21987921 | 21987971 |
| chr10 | 23606945 | 23606995 |
| chr10 | 1103757 | 1103807 |
| chr12 | 22488703 | 22488753 |
| chr12 | 25870042 | 25870092 |
| chr12 | 30457769 | 30457819 |
| chr12 | 30806774 | 30806824 |
| chr12 | 41380329 | 41380379 |
| chr12 | 46901752 | 46901802 |
| chr12 | 50205742 | 50205792 |
| chr12 | 56591105 | 56591155 |
| chr12 | 62960866 | 62960916 |
| chr12 | 73238994 | 73239044 |
| chr12 | 74349511 | 74349561 |
| chr12 | 84624791 | 84624841 |
| chr10 | 29463578 | 29463628 |
| chr12 | 88190443 | 88190493 |
| chr12 | 88757561 | 88757611 |
| chr12 | 89858467 | 89858517 |
| chr12 | 93824643 | 93824693 |
| chr12 | 107741709 | 107741759 |
| chr12 | 115490829 | 115490879 |
| chr12 | 116976780 | 116976830 |
| chr10 | 32517247 | 32517297 |
| chr12 | 123559989 | 123560039 |
| chr12 | 123572104 | 123572154 |
| chr12 | 124482877 | 124482927 |
| chr12 | 124489955 | 124490005 |
| chr12 | 124988593 | 124988643 |
| chr12 | 124990006 | 124990056 |
| chr12 | 126281682 | 126281732 |
| chr12 | 126881269 | 126881319 |
| chr12 | 128649374 | 128649424 |
| chr12 | 131166680 | 131166730 |
| chr13 | 29863915 | 29863965 |
| chr10 | 37212001 | 37212051 |
| chr13 | 32090042 | 32090092 |
| chr13 | 32462730 | 32462780 |
| chr13 | 34514930 | 34514980 |
| chr13 | 41019904 | 41019954 |
| chr10 | 38172937 | 38172987 |
| chr13 | 44949681 | 44949731 |
| chr13 | 44949176 | 44949226 |
| chr13 | 46310985 | 46311035 |
| chr13 | 49087193 | 49087243 |
| chr13 | 49242585 | 49242635 |
| chr13 | 64163906 | 64163956 |
| chr13 | 66060461 | 66060511 |
| chr13 | 76150254 | 76150304 |
| chr13 | 79571623 | 79571673 |
| chr13 | 84853777 | 84853827 |
| chr13 | 87688039 | 87688089 |
| chr13 | 88850597 | 88850647 |
| chr13 | 105537151 | 105537201 |
| chr13 | 109663122 | 109663172 |
| chr13 | 111275971 | 111276021 |
| chr10 | 47846115 | 47846165 |
| chr14 | 38561207 | 38561257 |
| chr14 | 43508332 | 43508382 |
| chr14 | 51358080 | 51358130 |
| chr14 | 62538575 | 62538625 |
| chr10 | 1866112 | 1866162 |
| chr10 | 1866058 | 1866108 |
| chr14 | 76218967 | 76219017 |
| chr14 | 76427788 | 76427838 |
| chr10 | 54803155 | 54803205 |
| chr14 | 89500945 | 89500995 |
| chr10 | 55777194 | 55777244 |
| chr14 | 93307052 | 93307102 |
| chr14 | 99713793 | 99713843 |
| chr14 | 99713554 | 99713604 |
| chr14 | 102669904 | 102669954 |
| chr14 | 102882202 | 102882252 |
| chr14 | 103480824 | 103480874 |
| chr14 | 103611335 | 103611385 |
| chr14 | 103726056 | 103726106 |
| chr15 | 20306201 | 20306251 |
| chr15 | 20783835 | 20783885 |
| chr15 | 22688962 | 22689012 |
| chr15 | 23695006 | 23695056 |
| chr15 | 23694805 | 23694855 |
| chr15 | 27394543 | 27394593 |
| chr15 | 29203465 | 29203515 |
| chr15 | 30654356 | 30654406 |
| chr15 | 36118709 | 36118759 |
| chr15 | 49313440 | 49313490 |
| chr15 | 53833211 | 53833261 |
| chr15 | 54621186 | 54621236 |
| chr10 | 66573792 | 66573842 |
| chr15 | 69048660 | 69048710 |
| chr15 | 88846391 | 88846441 |
| chr15 | 89300669 | 89300719 |
| chr16 | 439124 | 439174 |
| chr16 | 440001 | 440051 |
| chr10 | 70241432 | 70241482 |
| chr10 | 70241669 | 70241719 |
| chr16 | 5079413 | 5079463 |
| chr16 | 5879045 | 5879095 |
| chr16 | 6188804 | 6188854 |
| chr16 | 7512964 | 7513014 |
| chr16 | 16541151 | 16541201 |
| chr16 | 18020489 | 18020539 |
| chr16 | 18291725 | 18291775 |
| chr16 | 25297275 | 25297325 |
| chr16 | 25551808 | 25551858 |
| chr16 | 48213334 | 48213384 |
| chr16 | 56032184 | 56032234 |
| chr10 | 73265644 | 73265694 |
| chr16 | 59492617 | 59492667 |
| chr16 | 62512910 | 62512960 |
| chr16 | 62765086 | 62765136 |
| chr16 | 76448548 | 76448598 |
| chr16 | 77232267 | 77232317 |
| chr16 | 85819599 | 85819649 |
| chr10 | 75098284 | 75098334 |
| chr10 | 76069850 | 76069900 |
| chr10 | 76345327 | 76345377 |
| chr10 | 76442077 | 76442127 |
| chr10 | 79039069 | 79039119 |
| chr17 | 26390770 | 26390820 |
| chr17 | 29473237 | 29473287 |

Table 15DD. Repeat Category MLT1E2

| | | |
|---|---|---|
| chr17 | 75657698 | 75657748 |
| chr17 | 78800988 | 78801038 |
| chr17 | 79642713 | 79642763 |
| chr18 | 30045015 | 30045065 |
| chr18 | 30607603 | 30607653 |
| chr18 | 32655270 | 32655320 |
| chr18 | 32871641 | 32871691 |
| chr18 | 36514156 | 36514206 |
| chr18 | 49525078 | 49525128 |
| chr18 | 63077178 | 63077228 |
| chr18 | 65396154 | 65396204 |
| chr18 | 66710209 | 66710259 |
| chr18 | 73517328 | 73517378 |
| chr18 | 74265236 | 74265286 |
| chr19 | 329657 | 329707 |
| chr19 | 1118055 | 1118105 |
| chr19 | 1290155 | 1290205 |
| chr19 | 4290134 | 4290184 |
| chr19 | 4290360 | 4290410 |
| chr19 | 14260698 | 14260748 |
| chr19 | 21557352 | 21557402 |
| chr19 | 21556880 | 21556930 |
| chr19 | 47698558 | 47698608 |
| chr19 | 59678885 | 59678935 |
| chr1 | 9000468 | 9000518 |
| chr1 | 23841184 | 23841234 |
| chr1 | 37686990 | 37687040 |
| chr1 | 59489448 | 59489498 |
| chr1 | 64594611 | 64594661 |
| chr1 | 70284792 | 70284842 |
| chr1 | 85407219 | 85407269 |
| chr1 | 85407141 | 85407191 |
| chr1 | 160580408 | 160580458 |
| chr1 | 165297962 | 165298012 |
| chr1 | 166644595 | 166644645 |
| chr1 | 195544555 | 195544605 |
| chr1 | 216740268 | 216740318 |
| chr1 | 241770014 | 241770064 |
| chr1 | 243813655 | 243813705 |
| chr1 | 244547850 | 244547900 |
| chr20 | 669783 | 669833 |
| chr20 | 5642968 | 5643018 |
| chr20 | 44125679 | 44125729 |
| chr20 | 50616436 | 50616486 |
| chr20 | 56353794 | 56353844 |
| chr20 | 58396502 | 58396552 |
| chr20 | 59910057 | 59910107 |
| chr20 | 62140316 | 62140366 |
| chr21 | 10087446 | 10087496 |
| chr21 | 18272127 | 18272177 |
| chr21 | 22562168 | 22562218 |
| chr21 | 31324199 | 31324249 |
| chr21 | 31324811 | 31324861 |
| chr21 | 37571180 | 37571230 |
| chr21 | 44454007 | 44454057 |
| chr21 | 44454910 | 44454960 |
| chr21 | 44454737 | 44454787 |
| chr21 | 45073476 | 45073526 |
| chr21 | 45525063 | 45525113 |
| chr22 | 19189270 | 19189320 |
| chr22 | 31668958 | 31669008 |
| chr22 | 32137870 | 32137920 |
| chr22 | 34097825 | 34097875 |
| chr22 | 47854320 | 47854370 |
| chrX | 6809179 | 6809229 |
| chrX | 6809112 | 6809162 |
| chrX | 35290601 | 35290651 |
| chrX | 49663992 | 49664042 |
| chrX | 55961522 | 55961572 |
| chrX | 86355721 | 86355771 |
| chrX | 105149088 | 105149138 |
| chr11 | 10556227 | 10556277 |
| chr11 | 10556684 | 10556734 |
| chr11 | 10556750 | 10556800 |
| chrX | 130089934 | 130089984 |
| chrX | 130408767 | 130408817 |
| chrY | 4552353 | 4552403 |
| chr2 | 5543347 | 5543397 |
| chr11 | 17678055 | 17678105 |
| chr2 | 20895353 | 20895403 |
| chr2 | 81902922 | 81902972 |
| chr11 | 31030017 | 31030067 |
| chr2 | 132882192 | 132882242 |
| chr2 | 171933491 | 171933541 |
| chr2 | 196582938 | 196582988 |
| chr2 | 196584147 | 196584197 |
| chr2 | 208508603 | 208508653 |
| chr11 | 42337461 | 42337511 |
| chr2 | 225166052 | 225166102 |
| chr2 | 226019697 | 226019747 |
| chr2 | 229535737 | 229535787 |
| chr2 | 229621126 | 229621176 |
| chr3 | 3685495 | 3685545 |
| chr3 | 21700082 | 21700132 |
| chr3 | 42412715 | 42412765 |
| chr3 | 42412465 | 42412515 |
| chr3 | 59382295 | 59382345 |
| chr3 | 59382192 | 59382242 |
| chr3 | 77739751 | 77739801 |
| chr3 | 86881111 | 86881161 |
| chr3 | 95632183 | 95632233 |
| chr3 | 102908967 | 102909017 |
| chr3 | 127171332 | 127171382 |
| chr3 | 130057005 | 130057055 |
| chr3 | 137705213 | 137705263 |
| chr3 | 142968468 | 142968518 |
| chr3 | 142968378 | 142968428 |
| chr3 | 172320890 | 172320940 |
| chr11 | 63525125 | 63525175 |
| chr4 | 3775205 | 3775255 |
| chr4 | 21982637 | 21982687 |
| chr4 | 22497116 | 22497166 |
| chr4 | 33558999 | 33559049 |
| chr4 | 68344529 | 68344579 |
| chr4 | 81743745 | 81743795 |
| chr4 | 92045141 | 92045191 |
| chr4 | 131700058 | 131700108 |
| chr4 | 142706444 | 142706494 |
| chr4 | 142706241 | 142706291 |
| chr4 | 163844864 | 163844914 |
| chr4 | 169125144 | 169125194 |
| chr4 | 174542348 | 174542398 |
| chr4 | 177807252 | 177807302 |
| chr4 | 180740683 | 180740733 |
| chr4 | 184837525 | 184837575 |
| chr11 | 68590792 | 68590842 |
| chr5 | 15920994 | 15921044 |
| chr5 | 16866919 | 16866969 |
| chr5 | 22161951 | 22162001 |
| chr5 | 23370499 | 23370549 |
| chr5 | 37426668 | 37426718 |
| chr5 | 68149252 | 68149302 |
| chr5 | 68591236 | 68591286 |
| chr5 | 70768204 | 70768254 |
| chr5 | 78280975 | 78281025 |
| chr5 | 97894081 | 97894131 |
| chr5 | 108095800 | 108095850 |
| chr5 | 110087810 | 110087860 |
| chr5 | 114656062 | 114656112 |
| chr5 | 117365367 | 117365417 |
| chr5 | 123354510 | 123354560 |
| chr5 | 128976009 | 128976059 |
| chr5 | 177703831 | 177703881 |
| chr6 | 2302240 | 2302290 |
| chr6 | 5922901 | 5922951 |
| chr6 | 6226915 | 6226965 |
| chr6 | 10279733 | 10279783 |
| chr6 | 66948363 | 66948413 |
| chr6 | 84668687 | 84668737 |
| chr6 | 84668443 | 84668493 |
| chr6 | 103294987 | 103295037 |
| chr6 | 103462261 | 103462311 |
| chr6 | 113480283 | 113480333 |
| chr6 | 115232969 | 115233019 |
| chr6 | 146472859 | 146472909 |
| chr6 | 146472598 | 146472648 |
| chr6 | 150935692 | 150935742 |
| chr6 | 157931284 | 157931334 |
| chr6 | 164696499 | 164696549 |
| chr6 | 165343844 | 165343894 |
| chr6 | 165883566 | 165883616 |
| chr7 | 227093 | 227143 |
| chr7 | 3739170 | 3739220 |
| chr7 | 4598522 | 4598572 |
| chr7 | 4921226 | 4921276 |
| chr7 | 24478207 | 24478257 |
| chr7 | 44999757 | 44999807 |
| chr7 | 49886350 | 49886400 |
| chr7 | 63174518 | 63174568 |
| chr7 | 67727538 | 67727588 |
| chr7 | 90698367 | 90698417 |
| chr7 | 109179048 | 109179098 |
| chr7 | 135550670 | 135550720 |
| chr7 | 140941959 | 140942009 |
| chr8 | 4616838 | 4616888 |
| chr8 | 5452171 | 5452221 |
| chr11 | 124059447 | 124059497 |
| chr8 | 34602931 | 34602981 |
| chr8 | 41363422 | 41363472 |
| chr8 | 61489135 | 61489185 |
| chr11 | 125514648 | 125514698 |
| chr11 | 125819610 | 125819660 |
| chr8 | 107914805 | 107914855 |
| chr8 | 112720251 | 112720301 |
| chr8 | 118485921 | 118485971 |
| chr8 | 139902855 | 139902905 |
| chr8 | 142592164 | 142592214 |
| chr8 | 144314346 | 144314396 |
| chr9 | 2738496 | 2738546 |
| chr9 | 15083012 | 15083062 |
| chr9 | 23469176 | 23469226 |
| chr11 | 132823075 | 132823125 |
| chr9 | 30080616 | 30080666 |
| chr9 | 73253139 | 73253189 |
| chr9 | 92687906 | 92687956 |
| chr9 | 106050739 | 106050789 |
| chr9 | 119808920 | 119808970 |
| chr9 | 130941445 | 130941495 |
| chr9 | 134060962 | 134061012 |
| chr9 | 135706488 | 135706538 |
| chr10 | 21987921 | 21987971 |
| chr12 | 22488703 | 22488753 |
| chr12 | 25870042 | 25870092 |
| chr12 | 56591105 | 56591155 |
| chr12 | 88757561 | 88757611 |
| chr10 | 32517247 | 32517297 |
| chr12 | 124482877 | 124482927 |
| chr12 | 124489955 | 124490005 |
| chr12 | 124988593 | 124988643 |
| chr12 | 124990006 | 124990056 |
| chr12 | 128649374 | 128649424 |
| chr12 | 131166680 | 131166730 |
| chr10 | 37212001 | 37212051 |
| chr13 | 32090042 | 32090092 |
| chr13 | 46310985 | 46311035 |
| chr13 | 49087193 | 49087243 |
| chr13 | 84853777 | 84853827 |
| chr13 | 88850597 | 88850647 |
| chr13 | 105537151 | 105537201 |
| chr14 | 43508332 | 43508382 |
| chr14 | 51358080 | 51358130 |
| chr10 | 1866112 | 1866162 |
| chr10 | 1866058 | 1866108 |
| chr14 | 89500945 | 89500995 |
| chr14 | 102669904 | 102669954 |
| chr14 | 103480824 | 103480874 |
| chr14 | 103726056 | 103726106 |
| chr15 | 20783835 | 20783885 |
| chr15 | 49313440 | 49313490 |
| chr15 | 53833211 | 53833261 |
| chr16 | 5079413 | 5079463 |
| chr16 | 7512964 | 7513014 |
| chr16 | 48213334 | 48213384 |
| chr16 | 56032184 | 56032234 |
| chr10 | 73265644 | 73265694 |
| chr16 | 62512910 | 62512960 |
| chr16 | 85819599 | 85819649 |
| chr10 | 76069850 | 76069900 |
| chr10 | 79039069 | 79039119 |

Table 15DE. Repeat Category MLT1F

| | | |
|---|---|---|
| chr17 | 39071686 | 39071736 |
| chr17 | 45871163 | 45871213 |
| chr17 | 47838548 | 47838598 |
| chr17 | 59104693 | 59104743 |
| chr17 | 59217273 | 59217323 |
| chr17 | 67224841 | 67224891 |
| chr17 | 71743866 | 71743916 |
| chr17 | 72666581 | 72666631 |
| chr17 | 74034896 | 74034946 |
| chr17 | 74580745 | 74580795 |
| chr17 | 75164482 | 75164532 |
| chr17 | 76835361 | 76835411 |
| chr17 | 76836755 | 76836805 |
| chr17 | 80086590 | 80086640 |
| chr10 | 92621546 | 92621596 |
| chr17 | 80621480 | 80621530 |
| chr10 | 93909856 | 93909906 |
| chr18 | 11922659 | 11922709 |
| chr10 | 94974781 | 94974831 |
| chr18 | 22826965 | 22827015 |
| chr18 | 24382754 | 24382804 |
| chr10 | 95796346 | 95796396 |
| chr18 | 36675429 | 36675479 |
| chr18 | 36866944 | 36866994 |
| chr18 | 42028491 | 42028541 |
| chr18 | 46104346 | 46104396 |
| chr18 | 53440600 | 53440650 |
| chr18 | 53594732 | 53594782 |
| chr18 | 58068017 | 58068067 |
| chr18 | 58631226 | 58631276 |
| chr18 | 73817063 | 73817113 |
| chr18 | 75834630 | 75834680 |
| chr19 | 463625 | 463675 |
| chr19 | 2317879 | 2317929 |
| chr19 | 2611249 | 2611299 |
| chr19 | 2692173 | 2692223 |
| chr19 | 3612026 | 3612076 |
| chr19 | 4770977 | 4771027 |
| chr10 | 101648692 | 101648742 |
| chr10 | 101870440 | 101870490 |
| chr19 | 8795787 | 8795837 |
| chr19 | 8998136 | 8998186 |
| chr19 | 9183205 | 9183255 |
| chr19 | 12795021 | 12795071 |
| chr19 | 13712633 | 13712683 |
| chr19 | 17446363 | 17446413 |
| chr19 | 39558813 | 39558863 |
| chr19 | 41353556 | 41353606 |
| chr19 | 42727104 | 42727154 |
| chr19 | 45848729 | 45848779 |
| chr19 | 48593167 | 48593217 |
| chr19 | 48633585 | 48633635 |
| chr19 | 48633735 | 48633785 |
| chr10 | 107983467 | 107983517 |
| chr19 | 50398924 | 50398974 |
| chr19 | 51772260 | 51772310 |
| chr19 | 51772894 | 51772944 |
| chr19 | 53844730 | 53844780 |
| chr19 | 53973015 | 53973065 |
| chr19 | 55018409 | 55018459 |
| chr19 | 55039196 | 55039246 |
| chr19 | 59559100 | 59559150 |
| chr19 | 59602322 | 59602372 |
| chr19 | 60415782 | 60415832 |
| chr19 | 61598756 | 61598806 |
| chr19 | 63363298 | 63363348 |
| chr19 | 63568373 | 63568423 |
| chr1 | 3738472 | 3738522 |
| chr10 | 116031715 | 116031765 |
| chr1 | 6580096 | 6580146 |
| chr1 | 7633036 | 7633086 |
| chr1 | 9450768 | 9450818 |
| chr1 | 11551069 | 11551119 |
| chr1 | 14216346 | 14216396 |
| chr1 | 14400976 | 14401026 |
| chr1 | 14400777 | 14400827 |
| chr1 | 15440057 | 15440107 |
| chr1 | 16172507 | 16172557 |
| chr1 | 16172800 | 16172850 |
| chr1 | 22664179 | 22664229 |
| chr1 | 23571768 | 23571818 |
| chr1 | 24369619 | 24369669 |
| chr1 | 25784989 | 25785039 |
| chr1 | 27927355 | 27927405 |
| chr10 | 120555242 | 120555292 |
| chr1 | 29893133 | 29893183 |
| chr1 | 32500238 | 32500288 |
| chr1 | 63144111 | 63144161 |
| chr1 | 64232586 | 64232636 |
| chr10 | 6610343 | 6610393 |
| chr1 | 72722194 | 72722244 |
| chr1 | 73031388 | 73031438 |
| chr1 | 88978115 | 88978165 |
| chr1 | 89016961 | 89017011 |
| chr1 | 102593718 | 102593768 |
| chr1 | 111518427 | 111518477 |
| chr1 | 113009981 | 113010031 |
| chr1 | 114342224 | 114342274 |
| chr1 | 117852193 | 117852243 |
| chr10 | 127655663 | 127655713 |
| chr1 | 176475850 | 176475900 |
| chr1 | 187254490 | 187254540 |
| chr1 | 187924409 | 187924459 |
| chr1 | 200937916 | 200937966 |
| chr1 | 201094375 | 201094425 |
| chr1 | 210311915 | 210311965 |
| chr1 | 215853149 | 215853199 |
| chr1 | 216826289 | 216826339 |
| chr1 | 228974382 | 228974432 |
| chr1 | 235647318 | 235647368 |
| chr1 | 242105657 | 242105707 |
| chr1 | 242806821 | 242806871 |
| chr20 | 296177 | 296227 |
| chr20 | 802400 | 802450 |
| chr20 | 2559772 | 2559822 |
| chr20 | 5615696 | 5615746 |
| chr20 | 5797290 | 5797340 |
| chr20 | 5938312 | 5938362 |
| chr20 | 11533963 | 11534013 |
| chr20 | 13615816 | 13615866 |
| chr10 | 133069071 | 133069121 |
| chr20 | 15878700 | 15878750 |
| chr20 | 20389642 | 20389692 |
| chr20 | 31580933 | 31580983 |
| chr10 | 133624391 | 133624441 |
| chr20 | 36653150 | 36653200 |
| chr20 | 40223237 | 40223287 |
| chr20 | 43584565 | 43584615 |
| chr20 | 45812921 | 45812971 |
| chr20 | 49973245 | 49973295 |
| chr20 | 50636234 | 50636284 |
| chr20 | 53154699 | 53154749 |
| chr20 | 55595415 | 55595465 |
| chr20 | 62062694 | 62062744 |
| chr20 | 62346837 | 62346887 |
| chr21 | 15083139 | 15083189 |
| chr21 | 15939883 | 15939933 |
| chr21 | 20233422 | 20233472 |
| chr21 | 29393901 | 29393951 |
| chr21 | 30038454 | 30038504 |
| chr10 | 8342517 | 8342567 |
| chr21 | 32770629 | 32770679 |
| chr21 | 33228169 | 33228219 |
| chr21 | 36192720 | 36192770 |
| chr21 | 41906570 | 41906620 |
| chr21 | 43159949 | 43159999 |
| chr21 | 43160065 | 43160115 |
| chr21 | 43168233 | 43168283 |
| chr21 | 44121637 | 44121687 |
| chr21 | 44215130 | 44215180 |
| chr21 | 46307911 | 46307961 |
| chr22 | 16032014 | 16032064 |
| chr22 | 16309519 | 16309569 |
| chr22 | 23830018 | 23830068 |
| chr22 | 25947641 | 25947691 |
| chr22 | 29865663 | 29865713 |
| chr22 | 34759928 | 34759978 |
| chr22 | 37775243 | 37775293 |
| chr22 | 40138192 | 40138242 |
| chr22 | 41298475 | 41298525 |
| chr22 | 41803769 | 41803819 |
| chr22 | 42841855 | 42841905 |
| chr22 | 46526694 | 46526744 |
| chr22 | 48011758 | 48011808 |
| chr22 | 48075796 | 48075846 |
| chr22 | 48305361 | 48305411 |
| chrX | 13478055 | 13478105 |
| chrX | 14177519 | 14177569 |
| chrX | 17142311 | 17142361 |
| chrX | 23042867 | 23042917 |
| chrX | 23841302 | 23841352 |
| chrX | 37585931 | 37585981 |
| chrX | 39345216 | 39345266 |
| chrX | 39950710 | 39950760 |
| chrX | 42487008 | 42487058 |
| chr11 | 6998874 | 6998924 |
| chrX | 45576963 | 45577013 |
| chr11 | 7224408 | 7224458 |
| chr11 | 7224265 | 7224315 |
| chrX | 46789669 | 46789719 |
| chrX | 47467972 | 47468022 |
| chrX | 47905550 | 47905600 |
| chrX | 49068576 | 49068626 |
| chrX | 52184140 | 52184190 |
| chrX | 53166751 | 53166801 |
| chrX | 68240643 | 68240693 |
| chrX | 75982250 | 75982300 |
| chrX | 95565023 | 95565073 |
| chrX | 95709686 | 95709736 |
| chrX | 95760737 | 95760787 |
| chrX | 99473450 | 99473500 |
| chrX | 109817441 | 109817491 |
| chrX | 112745887 | 112745937 |
| chrX | 114849536 | 114849586 |
| chr11 | 10829493 | 10829543 |
| chrX | 147054063 | 147054113 |
| chrX | 148811891 | 148811941 |
| chrX | 151740030 | 151740080 |
| chrX | 152532071 | 152532121 |
| chr2 | 3267055 | 3267105 |
| chr2 | 8713031 | 8713081 |
| chr2 | 8712900 | 8712950 |
| chr2 | 25552423 | 25552473 |
| chr2 | 25552257 | 25552307 |
| chr2 | 32434102 | 32434152 |
| chr2 | 39347781 | 39347831 |
| chr2 | 39513076 | 39513126 |
| chr2 | 43038139 | 43038189 |
| chr2 | 43210108 | 43210158 |
| chr2 | 43209868 | 43209918 |
| chr2 | 43804502 | 43804552 |
| chr2 | 48815338 | 48815388 |
| chr2 | 53861149 | 53861199 |
| chr2 | 58589343 | 58589393 |
| chr2 | 61012801 | 61012851 |
| chr2 | 62610051 | 62610101 |
| chr11 | 23996085 | 23996135 |
| chr11 | 24400944 | 24400994 |
| chr11 | 24706487 | 24706537 |
| chr2 | 74335998 | 74336048 |
| chr2 | 74854815 | 74854865 |
| chr2 | 75843543 | 75843593 |
| chr11 | 26906526 | 26906576 |
| chr2 | 77329427 | 77329477 |
| chr2 | 78773086 | 78773136 |
| chr2 | 81729245 | 81729295 |
| chr2 | 85281450 | 85281500 |
| chr11 | 27783359 | 27783409 |
| chr2 | 86900893 | 86900943 |
| chr2 | 87303336 | 87303386 |
| chr2 | 88928806 | 88928856 |
| chr2 | 95369412 | 95369462 |
| chr2 | 96900864 | 96900914 |
| chr11 | 30123860 | 30123910 |
| chr11 | 31992190 | 31992240 |
| chr2 | 110474129 | 110474179 |
| chr2 | 110962301 | 110962351 |
| chr2 | 113111019 | 113111069 |
| chr2 | 113302667 | 113302717 |
| chr2 | 113784854 | 113784904 |
| chr2 | 119625761 | 119625811 |
| chr2 | 122662146 | 122662196 |
| chr2 | 129216610 | 129216660 |
| chr2 | 135054116 | 135054166 |
| chr2 | 136140851 | 136140901 |
| chr2 | 140962477 | 140962527 |
| chr2 | 141134765 | 141134815 |
| chr2 | 145205977 | 145206027 |
| chr2 | 152384223 | 152384273 |
| chr2 | 160006282 | 160006332 |
| chr2 | 170765919 | 170765969 |
| chr2 | 181085617 | 181085667 |
| chr2 | 193960514 | 193960564 |
| chr2 | 200971337 | 200971387 |
| chr2 | 203744139 | 203744189 |
| chr2 | 204384541 | 204384591 |
| chr2 | 216166647 | 216166697 |
| chr2 | 216959021 | 216959071 |
| chr2 | 220207353 | 220207403 |
| chr2 | 220207448 | 220207498 |
| chr2 | 222409950 | 222410000 |
| chr2 | 232352055 | 232352105 |
| chr2 | 234434348 | 234434398 |
| chr2 | 238054837 | 238054887 |
| chr2 | 239331752 | 239331802 |
| chr2 | 239421836 | 239421886 |
| chr2 | 241876510 | 241876560 |
| chr3 | 5250591 | 5250641 |
| chr3 | 9559873 | 9559923 |
| chr3 | 15668855 | 15668905 |
| chr3 | 26379414 | 26379464 |
| chr3 | 30439007 | 30439057 |
| chr3 | 38987586 | 38987636 |
| chr3 | 40525481 | 40525531 |
| chr3 | 44902314 | 44902364 |
| chr3 | 44902084 | 44902134 |
| chr3 | 46010834 | 46010884 |
| chr3 | 47926805 | 47926855 |
| chr3 | 49920556 | 49920606 |
| chr3 | 51612041 | 51612091 |
| chr3 | 57878943 | 57878993 |
| chr3 | 61224280 | 61224330 |
| chr3 | 62067849 | 62067899 |
| chr3 | 62067620 | 62067670 |
| chr3 | 65785787 | 65785837 |
| chr3 | 67697260 | 67697310 |
| chr3 | 68984356 | 68984406 |
| chr3 | 68984421 | 68984471 |
| chr3 | 101449845 | 101449895 |
| chr3 | 106260175 | 106260225 |
| chr3 | 107499192 | 107499242 |
| chr3 | 110461564 | 110461614 |
| chr3 | 112627691 | 112627741 |
| chr3 | 112691317 | 112691367 |
| chr3 | 114867932 | 114867982 |
| chr3 | 114867746 | 114867796 |
| chr3 | 125582950 | 125583000 |
| chr3 | 128911181 | 128911231 |
| chr3 | 129747404 | 129747454 |
| chr3 | 147103290 | 147103340 |
| chr3 | 147103117 | 147103167 |
| chr3 | 151407114 | 151407164 |
| chr3 | 159626951 | 159627001 |
| chr3 | 161518181 | 161518231 |
| chr3 | 162076618 | 162076668 |
| chr3 | 162309553 | 162309603 |
| chr10 | 12887684 | 12887734 |
| chr3 | 178298415 | 178298465 |
| chr3 | 184939170 | 184939220 |
| chr3 | 184940529 | 184940579 |
| chr3 | 188120135 | 188120185 |
| chr11 | 62987799 | 62987849 |
| chr3 | 192279985 | 192280035 |
| chr3 | 193877524 | 193877574 |
| chr4 | 694204 | 694254 |
| chr4 | 1613199 | 1613249 |
| chr4 | 2791685 | 2791735 |
| chr4 | 3338388 | 3338438 |
| chr4 | 4652175 | 4652225 |
| chr4 | 4651920 | 4651970 |
| chr4 | 7780612 | 7780662 |
| chr4 | 18028303 | 18028353 |
| chr4 | 20846736 | 20846786 |
| chr4 | 25454091 | 25454141 |
| chr4 | 36540280 | 36540330 |
| chr4 | 38493723 | 38493773 |
| chr4 | 39540115 | 39540165 |
| chr4 | 44781742 | 44781792 |
| chr4 | 46650075 | 46650125 |
| chr4 | 48047970 | 48048020 |
| chr4 | 54854841 | 54854891 |
| chr4 | 55921132 | 55921182 |
| chr4 | 57510914 | 57510964 |
| chr4 | 59077222 | 59077272 |
| chr4 | 60928132 | 60928182 |
| chr4 | 82109015 | 82109065 |
| chr4 | 84283546 | 84283596 |
| chr11 | 65716047 | 65716097 |
| chr4 | 86003460 | 86003510 |
| chr4 | 96101259 | 96101309 |
| chr4 | 97108007 | 97108057 |
| chr4 | 107045807 | 107045857 |
| chr4 | 107667990 | 107668040 |
| chr4 | 112504259 | 112504309 |
| chr4 | 114258881 | 114258931 |
| chr4 | 115231995 | 115232045 |
| chr4 | 121540640 | 121540690 |
| chr4 | 126352019 | 126352069 |
| chr4 | 129056103 | 129056153 |
| chr4 | 130092495 | 130092545 |
| chr4 | 130162847 | 130162897 |
| chr11 | 66511904 | 66511954 |
| chr4 | 133391388 | 133391438 |
| chr4 | 135297915 | 135297965 |
| chr4 | 151710205 | 151710255 |
| chr4 | 151709982 | 151710032 |
| chr4 | 152136944 | 152136994 |
| chr11 | 67089836 | 67089886 |
| chr4 | 160872855 | 160872905 |
| chr4 | 161477188 | 161477238 |
| chr4 | 166336872 | 166336922 |
| chr4 | 175532466 | 175532516 |
| chr4 | 178080590 | 178080640 |
| chr4 | 182706449 | 182706499 |
| chr4 | 188118314 | 188118364 |
| chr4 | 188118752 | 188118802 |
| chr4 | 188716207 | 188716257 |
| chr4 | 190254153 | 190254203 |
| chr5 | 245287 | 245337 |
| chr5 | 1014793 | 1014843 |
| chr5 | 1986979 | 1987029 |
| chr5 | 6075012 | 6075062 |
| chr5 | 8616246 | 8616296 |
| chr5 | 10657413 | 10657463 |
| chr5 | 21144909 | 21144959 |
| chr5 | 29312782 | 29312832 |
| chr5 | 32222282 | 32222332 |
| chr5 | 34978124 | 34978174 |
| chr5 | 35578581 | 35578631 |
| chr5 | 38546889 | 38546939 |
| chr5 | 39371066 | 39371116 |
| chr5 | 45483914 | 45483964 |
| chr5 | 53794012 | 53794062 |
| chr5 | 58180120 | 58180170 |
| chr11 | 70471508 | 70471558 |
| chr5 | 71002822 | 71002872 |
| chr5 | 74061942 | 74061992 |
| chr5 | 74917467 | 74917517 |
| chr5 | 76304865 | 76304915 |
| chr5 | 76501677 | 76501727 |
| chr5 | 84377335 | 84377385 |
| chr5 | 85513802 | 85513852 |
| chr5 | 103113882 | 103113932 |
| chr5 | 106157293 | 106157343 |
| chr5 | 112068206 | 112068256 |
| chr5 | 112480144 | 112480194 |
| chr5 | 119159793 | 119159843 |
| chr5 | 122147062 | 122147112 |
| chr5 | 126218141 | 126218191 |
| chr5 | 130569114 | 130569164 |
| chr5 | 130921674 | 130921724 |
| chr5 | 132426356 | 132426406 |
| chr5 | 134225133 | 134225183 |
| chr5 | 138132013 | 138132063 |
| chr5 | 138133653 | 138133703 |
| chr5 | 138199022 | 138199072 |
| chr5 | 138347842 | 138347892 |
| chr5 | 138668032 | 138668082 |
| chr5 | 139387481 | 139387531 |
| chr5 | 139488762 | 139488812 |
| chr5 | 139501048 | 139501098 |
| chr5 | 139609129 | 139609179 |
| chr5 | 140111602 | 140111652 |
| chr5 | 141459637 | 141459687 |
| chr5 | 156497695 | 156497745 |
| chr5 | 171892389 | 171892439 |
| chr5 | 172965556 | 172965606 |
| chr5 | 174626794 | 174626844 |
| chr5 | 175327127 | 175327177 |
| chr5 | 176483897 | 176483947 |
| chr5 | 179933852 | 179933902 |
| chr5 | 180689794 | 180689844 |
| chr6 | 1191585 | 1191635 |
| chr6 | 2902347 | 2902397 |
| chr6 | 3817648 | 3817698 |
| chr6 | 5012208 | 5012258 |
| chr6 | 6552255 | 6552305 |
| chr6 | 13609268 | 13609318 |
| chr6 | 13872648 | 13872698 |
| chr6 | 21178283 | 21178333 |
| chr6 | 21391929 | 21391979 |
| chr6 | 27177635 | 27177685 |
| chr6 | 27801900 | 27801950 |
| chr6 | 27935743 | 27935793 |
| chr10 | 15172921 | 15172971 |
| chr6 | 33695868 | 33695918 |
| chr6 | 35367103 | 35367153 |
| chr6 | 35772061 | 35772111 |
| chr6 | 36016047 | 36016097 |
| chr6 | 41103097 | 41103147 |
| chr6 | 41462319 | 41462369 |
| chr11 | 86629243 | 86629293 |
| chr6 | 64300189 | 64300239 |
| chr6 | 66023691 | 66023741 |
| chr6 | 68982595 | 68982645 |
| chr6 | 69334461 | 69334511 |
| chr6 | 70306607 | 70306657 |
| chr11 | 89874411 | 89874461 |
| chr6 | 74026423 | 74026473 |
| chr11 | 91307903 | 91307953 |
| chr6 | 108217708 | 108217758 |
| chr6 | 115056135 | 115056185 |
| chr6 | 116842471 | 116842521 |
| chr6 | 128076336 | 128076386 |
| chr6 | 139430899 | 139430949 |
| chr6 | 163964464 | 163964514 |
| chr6 | 164756639 | 164756689 |
| chr6 | 167307759 | 167307809 |
| chr7 | 1613084 | 1613134 |
| chr11 | 107707516 | 107707566 |
| chr7 | 4456539 | 4456589 |
| chr7 | 5861127 | 5861177 |
| chr7 | 25929316 | 25929366 |
| chr7 | 26654295 | 26654345 |
| chr7 | 35052296 | 35052346 |
| chr7 | 35761667 | 35761717 |
| chr7 | 38595062 | 38595112 |
| chr7 | 54791547 | 54791597 |
| chr7 | 56597365 | 56597415 |
| chr7 | 65009545 | 65009595 |
| chr7 | 71164829 | 71164879 |
| chr7 | 72952260 | 72952310 |
| chr7 | 82311705 | 82311755 |
| chr7 | 92846028 | 92846078 |
| chr7 | 97882914 | 97882964 |
| chr7 | 99718424 | 99718474 |
| chr7 | 105303415 | 105303465 |
| chr7 | 105372233 | 105372283 |
| chr7 | 125051716 | 125051766 |
| chr7 | 132326854 | 132326904 |
| chr7 | 138782480 | 138782530 |
| chr7 | 139576426 | 139576476 |
| chr7 | 139577352 | 139577402 |
| chr7 | 147978484 | 147978534 |
| chr7 | 148137297 | 148137347 |
| chr7 | 152822916 | 152822966 |
| chr7 | 153186761 | 153186811 |
| chr7 | 153811053 | 153811103 |
| chr7 | 156330857 | 156330907 |
| chr7 | 156360651 | 156360701 |
| chr7 | 157189184 | 157189234 |
| chr8 | 23599 | 23649 |
| chr8 | 1914259 | 1914309 |
| chr8 | 9101514 | 9101564 |
| chr8 | 9654580 | 9654630 |
| chr8 | 10042674 | 10042724 |
| chr8 | 13250066 | 13250116 |
| chr8 | 15018941 | 15018991 |
| chr11 | 122488344 | 122488394 |
| chr8 | 22974244 | 22974294 |
| chr8 | 23239401 | 23239451 |
| chr8 | 28644731 | 28644781 |
| chr8 | 30685352 | 30685402 |
| chr8 | 34877997 | 34878047 |
| chr8 | 37978050 | 37978100 |
| chr8 | 42914443 | 42914493 |
| chr8 | 42917019 | 42917069 |
| chr8 | 60448890 | 60448940 |
| chr11 | 125584444 | 125584494 |
| chr8 | 65807715 | 65807765 |
| chr8 | 66602636 | 66602686 |
| chr8 | 66920969 | 66921019 |
| chr8 | 74797544 | 74797594 |
| chr8 | 89732082 | 89732132 |
| chr8 | 90676737 | 90676787 |
| chr8 | 95246983 | 95247033 |
| chr8 | 95936910 | 95936960 |
| chr8 | 102444287 | 102444337 |
| chr8 | 103586082 | 103586132 |
| chr8 | 119815714 | 119815764 |
| chr8 | 127565096 | 127565146 |
| chr8 | 128425710 | 128425760 |
| chr8 | 131516408 | 131516458 |
| chr8 | 140383523 | 140383573 |
| chr8 | 142227483 | 142227533 |
| chr8 | 142369390 | 142369440 |
| chr8 | 142715550 | 142715600 |
| chr8 | 144357831 | 144357881 |
| chr8 | 144423722 | 144423772 |
| chr8 | 144423433 | 144423483 |
| chr8 | 145358253 | 145358303 |
| chr9 | 483208 | 483258 |
| chr9 | 1799869 | 1799919 |
| chr9 | 2346635 | 2346685 |
| chr9 | 6796826 | 6796876 |
| chr11 | 132143103 | 132143153 |
| chr9 | 12944109 | 12944159 |
| chr9 | 17830955 | 17831005 |
| chr9 | 20319218 | 20319268 |
| chr9 | 38367567 | 38367617 |
| chr9 | 38368829 | 38368879 |
| chr9 | 72976522 | 72976572 |
| chr9 | 77808855 | 77808905 |
| chr9 | 85491422 | 85491472 |
| chr9 | 86654526 | 86654576 |
| chr9 | 91193443 | 91193493 |
| chr9 | 94577013 | 94577063 |
| chr9 | 96960735 | 96960785 |
| chr9 | 105259066 | 105259116 |
| chr9 | 115990107 | 115990157 |
| chr9 | 115991545 | 115991595 |
| chr12 | 1359635 | 1359685 |
| chr9 | 124895644 | 124895694 |
| chr9 | 126154431 | 126154481 |
| chr9 | 129105052 | 129105102 |
| chr9 | 129995736 | 129995786 |
| chr9 | 131297963 | 131298013 |
| chr9 | 133171727 | 133171777 |
| chr9 | 133887157 | 133887207 |
| chr12 | 6129080 | 6129130 |
| chr12 | 6761610 | 6761660 |
| chr12 | 7692419 | 7692469 |
| chr12 | 8317135 | 8317185 |
| chr12 | 25408389 | 25408439 |
| chr12 | 29746873 | 29746923 |
| chr10 | 24988124 | 24988174 |
| chr12 | 40959838 | 40959888 |
| chr12 | 45056630 | 45056680 |
| chr12 | 45128295 | 45128345 |
| chr12 | 47418403 | 47418453 |
| chr12 | 50539003 | 50539053 |
| chr12 | 56968840 | 56968890 |
| chr12 | 60986043 | 60986093 |
| chr12 | 64837889 | 64837939 |
| chr12 | 67213853 | 67213903 |
| chr12 | 75231144 | 75231194 |
| chr12 | 78844699 | 78844749 |
| chr12 | 89693542 | 89693592 |
| chr12 | 92860780 | 92860830 |
| chr12 | 98170282 | 98170332 |
| chr12 | 101985034 | 101985084 |
| chr12 | 103293857 | 103293907 |
| chr12 | 107656363 | 107656413 |
| chr12 | 107816070 | 107816120 |
| chr12 | 107815796 | 107815846 |
| chr12 | 110249371 | 110249421 |
| chr12 | 113381681 | 113381731 |
| chr12 | 115906409 | 115906459 |
| chr12 | 116927846 | 116927896 |
| chr12 | 118258205 | 118258255 |
| chr10 | 32625956 | 32626006 |
| chr12 | 121602372 | 121602422 |
| chr12 | 124327960 | 124328010 |
| chr10 | 35647061 | 35647111 |
| chr13 | 34067414 | 34067464 |
| chr13 | 34068287 | 34068337 |
| chr13 | 37636524 | 37636574 |
| chr13 | 43194669 | 43194719 |
| chr13 | 47798908 | 47798958 |
| chr13 | 48870349 | 48870399 |
| chr13 | 50416978 | 50417028 |
| chr13 | 50497385 | 50497435 |
| chr13 | 58134482 | 58134532 |
| chr13 | 58491566 | 58491616 |
| chr13 | 64357981 | 64358031 |
| chr13 | 66004607 | 66004657 |
| chr13 | 83519773 | 83519823 |
| chr13 | 88837668 | 88837718 |
| chr13 | 94696885 | 94696935 |
| chr13 | 96489160 | 96489210 |
| chr13 | 111049333 | 111049383 |
| chr13 | 112203444 | 112203494 |
| chr13 | 112208088 | 112208138 |
| chr14 | 21317056 | 21317106 |
| chr14 | 32444317 | 32444367 |
| chr14 | 35663309 | 35663359 |
| chr14 | 42727447 | 42727497 |
| chr14 | 43806534 | 43806584 |
| chr14 | 48832161 | 48832211 |
| chr14 | 52829932 | 52829982 |
| chr14 | 53641855 | 53641905 |
| chr14 | 57067371 | 57067421 |
| chr14 | 59195724 | 59195774 |
| chr14 | 71096027 | 71096077 |
| chr14 | 81288037 | 81288087 |
| chr14 | 81538508 | 81538558 |
| chr14 | 82083942 | 82083992 |
| chr14 | 83809998 | 83810048 |
| chr14 | 93057642 | 93057692 |
| chr14 | 94345214 | 94345264 |
| chr14 | 98772271 | 98772321 |
| chr10 | 57963877 | 57963927 |
| chr14 | 100998909 | 100998959 |
| chr14 | 103089988 | 103090038 |
| chr15 | 21712040 | 21712090 |
| chr15 | 25987247 | 25987297 |
| chr15 | 27093839 | 27093889 |
| chr15 | 27858164 | 27858214 |
| chr15 | 29693425 | 29693475 |
| chr15 | 30752514 | 30752564 |
| chr15 | 30771380 | 30771430 |
| chr10 | 63292902 | 63292952 |
| chr15 | 39044626 | 39044676 |
| chr15 | 39171556 | 39171606 |
| chr15 | 43217726 | 43217776 |
| chr15 | 48849597 | 48849647 |
| chr15 | 49985725 | 49985775 |
| chr15 | 61025214 | 61025264 |
| chr15 | 61151883 | 61151933 |
| chr15 | 63242930 | 63242980 |
| chr15 | 63572807 | 63572857 |
| chr15 | 69550711 | 69550761 |
| chr15 | 70816629 | 70816679 |
| chr15 | 73861885 | 73861935 |
| chr15 | 75647642 | 75647692 |
| chr15 | 83638845 | 83638895 |
| chr15 | 85494079 | 85494129 |
| chr15 | 87793860 | 87793910 |
| chr15 | 88214441 | 88214491 |
| chr15 | 88622338 | 88622388 |
| chr10 | 69759189 | 69759239 |
| chr15 | 94654294 | 94654344 |
| chr15 | 97230038 | 97230088 |
| chr10 | 70104088 | 70104138 |
| chr16 | 226967 | 227017 |
| chr16 | 1027708 | 1027758 |
| chr16 | 1027451 | 1027501 |
| chr16 | 2391658 | 2391708 |
| chr16 | 2406338 | 2406388 |
| chr16 | 2725773 | 2725823 |
| chr16 | 3180386 | 3180436 |
| chr16 | 4696365 | 4696415 |
| chr16 | 5535686 | 5535736 |
| chr16 | 5879045 | 5879095 |
| chr16 | 7007810 | 7007860 |
| chr16 | 9131602 | 9131652 |
| chr16 | 9460990 | 9461040 |
| chr16 | 10427595 | 10427645 |
| chr16 | 10586268 | 10586318 |
| chr16 | 10858574 | 10858624 |
| chr16 | 11915889 | 11915939 |
| chr16 | 12436426 | 12436476 |
| chr16 | 23522525 | 23522575 |
| chr16 | 29966171 | 29966221 |
| chr16 | 29966305 | 29966355 |
| chr16 | 31103357 | 31103407 |
| chr16 | 31149141 | 31149191 |
| chr16 | 46656260 | 46656310 |
| chr16 | 46662249 | 46662299 |
| chr16 | 56411872 | 56411922 |
| chr16 | 58081119 | 58081169 |
| chr16 | 60113353 | 60113403 |
| chr16 | 62140586 | 62140636 |
| chr16 | 63155437 | 63155487 |
| chr16 | 65500059 | 65500109 |
| chr16 | 66912749 | 66912799 |
| chr16 | 68404424 | 68404474 |
| chr16 | 69314198 | 69314248 |
| chr10 | 73953280 | 73953330 |
| chr16 | 75212195 | 75212245 |
| chr16 | 76723118 | 76723168 |
| chr16 | 80441908 | 80441958 |
| chr16 | 80441609 | 80441659 |
| chr16 | 85095814 | 85095864 |
| chr16 | 85215597 | 85215647 |
| chr16 | 87107657 | 87107707 |
| chr16 | 87107326 | 87107376 |
| chr16 | 87652684 | 87652734 |
| chr16 | 89140641 | 89140691 |
| chr16 | 89312607 | 89312657 |
| chr16 | 89671823 | 89671873 |
| chr17 | 307644 | 307694 |
| chr17 | 309190 | 309240 |
| chr10 | 76307542 | 76307592 |
| chr17 | 3726180 | 3726230 |
| chr17 | 4754733 | 4754783 |
| chr17 | 6279672 | 6279722 |
| chr17 | 6280400 | 6280450 |
| chr17 | 8820911 | 8820961 |
| chr17 | 10094251 | 10094301 |
| chr17 | 11533046 | 11533096 |
| chr17 | 14948401 | 14948451 |
| chr17 | 15529594 | 15529644 |
| chr17 | 16562294 | 16562344 |
| chr17 | 17547553 | 17547603 |
| chr17 | 32185059 | 32185109 |

Table 15DF. Repeat Category MLT1F1

| | | |
|---|---|---|
| chr17 | 47838548 | 47838598 |
| chr17 | 59104693 | 59104743 |
| chr17 | 75164482 | 75164532 |
| chr10 | 92621546 | 92621596 |
| chr17 | 80621480 | 80621530 |
| chr10 | 95796346 | 95796396 |
| chr18 | 36675429 | 36675479 |
| chr18 | 42028491 | 42028541 |
| chr18 | 58631226 | 58631276 |
| chr18 | 73817063 | 73817113 |
| chr19 | 463625 | 463675 |
| chr19 | 2317879 | 2317929 |
| chr19 | 2611249 | 2611299 |
| chr10 | 101648692 | 101648742 |
| chr19 | 12795021 | 12795071 |
| chr10 | 107983467 | 107983517 |
| chr19 | 51772260 | 51772310 |
| chr19 | 51772894 | 51772944 |
| chr19 | 60415782 | 60415832 |
| chr19 | 63568373 | 63568423 |
| chr10 | 116031715 | 116031765 |
| chr1 | 7633036 | 7633086 |
| chr1 | 9450768 | 9450818 |
| chr1 | 23571768 | 23571818 |
| chr1 | 24369619 | 24369669 |
| chr1 | 25784989 | 25785039 |
| chr10 | 120555242 | 120555292 |
| chr1 | 63144111 | 63144161 |
| chr10 | 127655663 | 127655713 |
| chr1 | 187254490 | 187254540 |
| chr1 | 228974382 | 228974432 |
| chr20 | 296177 | 296227 |
| chr20 | 5615696 | 5615746 |
| chr20 | 5938312 | 5938362 |
| chr10 | 133069071 | 133069121 |
| chr20 | 20389642 | 20389692 |
| chr10 | 133624391 | 133624441 |
| chr20 | 36653150 | 36653200 |
| chr20 | 40223237 | 40223287 |
| chr20 | 43584565 | 43584615 |
| chr20 | 55595415 | 55595465 |
| chr20 | 62346837 | 62346887 |
| chr21 | 15939883 | 15939933 |
| chr21 | 44121637 | 44121687 |
| chr21 | 44215130 | 44215180 |
| chr22 | 29865663 | 29865713 |
| chr22 | 40138192 | 40138242 |
| chr22 | 41803769 | 41803819 |
| chr22 | 48075796 | 48075846 |
| chr22 | 48305361 | 48305411 |
| chrX | 14177519 | 14177569 |
| chrX | 17142311 | 17142361 |
| chrX | 23841302 | 23841352 |
| chrX | 39950710 | 39950760 |
| chrX | 42487008 | 42487058 |
| chrX | 45576963 | 45577013 |
| chrX | 47905550 | 47905600 |
| chrX | 68240643 | 68240693 |
| chrX | 75982250 | 75982300 |
| chrX | 99473450 | 99473500 |
| chrX | 151740030 | 151740080 |
| chrX | 152532071 | 152532121 |
| chr2 | 32434102 | 32434152 |
| chr2 | 48815338 | 48815388 |
| chr2 | 53861149 | 53861199 |
| chr2 | 74335998 | 74336048 |
| chr2 | 78773086 | 78773136 |
| chr2 | 86900893 | 86900943 |
| chr2 | 113111019 | 113111069 |
| chr2 | 129216610 | 129216660 |
| chr2 | 160006282 | 160006332 |
| chr2 | 170765919 | 170765969 |
| chr2 | 200971337 | 200971387 |
| chr2 | 216166647 | 216166697 |
| chr2 | 220207353 | 220207403 |
| chr2 | 220207448 | 220207498 |
| chr2 | 234434348 | 234434398 |
| chr2 | 238054837 | 238054887 |
| chr2 | 239331752 | 239331802 |
| chr3 | 9559873 | 9559923 |
| chr3 | 26379414 | 26379464 |
| chr3 | 46010834 | 46010884 |
| chr3 | 61224280 | 61224330 |
| chr3 | 68984356 | 68984406 |
| chr3 | 68984421 | 68984471 |
| chr3 | 107499192 | 107499242 |
| chr3 | 110461564 | 110461614 |
| chr3 | 112627691 | 112627741 |
| chr3 | 125582950 | 125583000 |
| chr3 | 128911181 | 128911231 |
| chr3 | 162076618 | 162076668 |
| chr11 | 62987799 | 62987849 |
| chr3 | 193877524 | 193877574 |
| chr4 | 1613199 | 1613249 |
| chr4 | 7780612 | 7780662 |
| chr4 | 25454091 | 25454141 |
| chr4 | 84283546 | 84283596 |
| chr4 | 96101259 | 96101309 |
| chr4 | 107045807 | 107045857 |
| chr4 | 115231995 | 115232045 |
| chr4 | 121540640 | 121540690 |
| chr4 | 129056103 | 129056153 |
| chr4 | 130092495 | 130092545 |
| chr4 | 133391388 | 133391438 |
| chr11 | 67089836 | 67089886 |
| chr4 | 161477188 | 161477238 |
| chr4 | 175532466 | 175532516 |
| chr4 | 178080590 | 178080640 |
| chr4 | 190254153 | 190254203 |
| chr5 | 1014793 | 1014843 |
| chr5 | 6075012 | 6075062 |
| chr5 | 32222282 | 32222332 |
| chr5 | 53794012 | 53794062 |
| chr5 | 58180120 | 58180170 |
| chr5 | 74061942 | 74061992 |
| chr5 | 74917467 | 74917517 |
| chr5 | 76501677 | 76501727 |
| chr5 | 85513802 | 85513852 |
| chr5 | 103113882 | 103113932 |
| chr5 | 112068206 | 112068256 |
| chr5 | 132426356 | 132426406 |
| chr5 | 138132013 | 138132063 |
| chr5 | 138199022 | 138199072 |
| chr5 | 138668032 | 138668082 |
| chr5 | 139488762 | 139488812 |
| chr5 | 139501048 | 139501098 |
| chr5 | 141459637 | 141459687 |
| chr5 | 172965556 | 172965606 |
| chr5 | 174626794 | 174626844 |
| chr5 | 175327127 | 175327177 |
| chr5 | 176483897 | 176483947 |
| chr5 | 180689794 | 180689844 |
| chr11 | 86629243 | 86629293 |
| chr6 | 70306607 | 70306657 |
| chr6 | 74026423 | 74026473 |
| chr11 | 91307903 | 91307953 |
| chr6 | 115056135 | 115056185 |
| chr6 | 116842471 | 116842521 |
| chr11 | 107707516 | 107707566 |
| chr7 | 38595062 | 38595112 |
| chr7 | 65009545 | 65009595 |
| chr7 | 71164829 | 71164879 |
| chr7 | 82311705 | 82311755 |
| chr7 | 105303415 | 105303465 |
| chr7 | 125051716 | 125051766 |
| chr7 | 139576426 | 139576476 |
| chr7 | 139577352 | 139577402 |
| chr7 | 156330857 | 156330907 |
| chr7 | 157189184 | 157189234 |
| chr8 | 23599 | 23649 |
| chr11 | 122488344 | 122488394 |
| chr8 | 30685352 | 30685402 |
| chr8 | 127565096 | 127565146 |
| chr8 | 144423722 | 144423772 |
| chr8 | 144423433 | 144423483 |
| chr9 | 1799869 | 1799919 |
| chr9 | 6796826 | 6796876 |
| chr9 | 12944109 | 12944159 |
| chr9 | 129105052 | 129105102 |
| chr9 | 129995736 | 129995786 |
| chr9 | 133171727 | 133171777 |
| chr9 | 133887157 | 133887207 |
| chr12 | 47418403 | 47418453 |
| chr12 | 50539003 | 50539053 |
| chr12 | 92860780 | 92860830 |
| chr12 | 110249371 | 110249421 |
| chr12 | 116927846 | 116927896 |
| chr10 | 32625956 | 32626006 |
| chr12 | 121602372 | 121602422 |
| chr12 | 124327960 | 124328010 |
| chr13 | 88837668 | 88837718 |
| chr14 | 48832161 | 48832211 |
| chr14 | 81538508 | 81538558 |
| chr14 | 82083942 | 82083992 |
| chr14 | 93057642 | 93057692 |
| chr14 | 94345214 | 94345264 |
| chr14 | 98772271 | 98772321 |
| chr14 | 100998909 | 100998959 |
| chr15 | 27858164 | 27858214 |
| chr15 | 30752514 | 30752564 |
| chr10 | 63292902 | 63292952 |
| chr15 | 39171556 | 39171606 |
| chr15 | 43217726 | 43217776 |
| chr15 | 63242930 | 63242980 |
| chr15 | 69550711 | 69550761 |
| chr10 | 69759189 | 69759239 |
| chr15 | 94654294 | 94654344 |
| chr16 | 226967 | 227017 |
| chr16 | 1027708 | 1027758 |
| chr16 | 1027451 | 1027501 |
| chr16 | 10586268 | 10586318 |
| chr16 | 11915889 | 11915939 |
| chr16 | 46656260 | 46656310 |
| chr16 | 46662249 | 46662299 |
| chr16 | 58081119 | 58081169 |
| chr16 | 66912749 | 66912799 |
| chr16 | 85095814 | 85095864 |
| chr16 | 87652684 | 87652734 |
| chr16 | 89140641 | 89140691 |
| chr16 | 89671823 | 89671873 |
| chr10 | 76307542 | 76307592 |
| chr17 | 8820911 | 8820961 |
| chr17 | 15529594 | 15529644 |
| chr17 | 16562294 | 16562344 |

Table 15DG. Repeat Category MLT1G1

| | | |
|---|---|---|
| chr17 | 58954765 | 58954815 |
| chr10 | 86187299 | 86187349 |
| chr17 | 72717269 | 72717319 |
| chr17 | 75547923 | 75547973 |
| chr17 | 75595937 | 75595987 |
| chr17 | 77557056 | 77557106 |
| chr18 | 11547510 | 11547560 |
| chr10 | 95129757 | 95129807 |
| chr10 | 95413321 | 95413371 |
| chr18 | 23406852 | 23406902 |
| chr18 | 36866944 | 36866994 |
| chr10 | 97933975 | 97934025 |
| chr18 | 69075067 | 69075117 |
| chr18 | 69794363 | 69794413 |
| chr18 | 73331526 | 73331576 |
| chr18 | 73331270 | 73331320 |
| chr10 | 100251131 | 100251181 |
| chr19 | 2838149 | 2838199 |
| chr10 | 101102945 | 101102995 |
| chr19 | 6848179 | 6848229 |
| chr19 | 6847789 | 6847839 |
| chr19 | 15530607 | 15530657 |
| chr19 | 44208004 | 44208054 |
| chr19 | 45216299 | 45216349 |
| chr19 | 45510386 | 45510436 |
| chr19 | 45848729 | 45848779 |
| chr19 | 46645050 | 46645100 |
| chr19 | 53719229 | 53719279 |
| chr1 | 9956135 | 9956185 |
| chr1 | 9958461 | 9958511 |
| chr1 | 10973375 | 10973425 |
| chr1 | 22117570 | 22117620 |
| chr1 | 24890233 | 24890283 |
| chr1 | 32673587 | 32673637 |
| chr1 | 38485694 | 38485744 |
| chr1 | 40198973 | 40199023 |
| chr1 | 40933098 | 40933148 |
| chr1 | 57315426 | 57315476 |
| chr1 | 63063337 | 63063387 |
| chr1 | 63063549 | 63063599 |
| chr1 | 63063932 | 63063982 |
| chr1 | 63064161 | 63064211 |
| chr1 | 93723638 | 93723688 |
| chr1 | 158073478 | 158073528 |
| chr1 | 167406656 | 167406706 |
| chr1 | 167407601 | 167407651 |
| chr1 | 179318425 | 179318475 |
| chr1 | 226348786 | 226348836 |
| chr1 | 244068010 | 244068060 |
| chr20 | 20052080 | 20052130 |
| chr20 | 20051962 | 20052012 |
| chr20 | 24784053 | 24784103 |
| chr20 | 24894030 | 24894080 |
| chr20 | 34373693 | 34373743 |
| chr20 | 58189827 | 58189877 |
| chr20 | 59507667 | 59507717 |
| chr20 | 62346837 | 62346887 |
| chr20 | 62749255 | 62749305 |
| chr21 | 21474200 | 21474250 |
| chr21 | 21473974 | 21474024 |
| chr21 | 26165546 | 26165596 |
| chr21 | 31890529 | 31890579 |
| chr21 | 34834766 | 34834816 |
| chr21 | 39706626 | 39706676 |
| chr21 | 40334791 | 40334841 |
| chr21 | 45349143 | 45349193 |
| chr21 | 46113059 | 46113109 |
| chr21 | 46113659 | 46113709 |
| chr22 | 24767594 | 24767644 |
| chr22 | 36789048 | 36789098 |
| chr22 | 36873158 | 36873208 |
| chr11 | 2561822 | 2561872 |
| chr11 | 2863193 | 2863243 |
| chrX | 17796167 | 17796217 |
| chrX | 21713834 | 21713884 |
| chrX | 23194469 | 23194519 |
| chrX | 38873474 | 38873524 |
| chr11 | 8654721 | 8654771 |
| chr2 | 645217 | 645267 |
| chr2 | 7194792 | 7194842 |
| chr2 | 8864628 | 8864678 |
| chr2 | 15233171 | 15233221 |
| chr2 | 16048807 | 16048857 |
| chr2 | 19652981 | 19653031 |
| chr2 | 25837239 | 25837289 |
| chr2 | 41526171 | 41526221 |
| chr2 | 69866988 | 69867038 |
| chr2 | 75799728 | 75799778 |
| chr2 | 112051140 | 112051190 |
| chr2 | 113784854 | 113784904 |
| chr2 | 120666259 | 120666309 |
| chr2 | 159225768 | 159225818 |
| chr2 | 159225520 | 159225570 |
| chr2 | 208574556 | 208574606 |
| chr2 | 215958477 | 215958527 |
| chr2 | 233696531 | 233696581 |
| chr2 | 236326913 | 236326963 |
| chr3 | 6587683 | 6587733 |
| chr3 | 14902067 | 14902117 |
| chr3 | 15362495 | 15362545 |
| chr3 | 20332646 | 20332696 |
| chr3 | 39173219 | 39173269 |
| chr3 | 46010834 | 46010884 |
| chr3 | 51612041 | 51612091 |
| chr3 | 78193364 | 78193414 |
| chr3 | 100845901 | 100845951 |
| chr3 | 100845236 | 100845286 |
| chr3 | 126257021 | 126257071 |
| chr3 | 127097883 | 127097933 |
| chr3 | 141758515 | 141758565 |
| chr3 | 166958907 | 166958957 |
| chr4 | 6734614 | 6734664 |
| chr4 | 7659924 | 7659974 |
| chr4 | 19466589 | 19466639 |
| chr4 | 29850644 | 29850694 |
| chr4 | 37615942 | 37615992 |
| chr4 | 37618923 | 37618973 |
| chr4 | 40841592 | 40841642 |
| chr4 | 44492212 | 44492262 |
| chr4 | 57767143 | 57767193 |
| chr4 | 89326161 | 89326211 |
| chr4 | 102210187 | 102210237 |
| chr4 | 110061801 | 110061851 |
| chr4 | 141088996 | 141089046 |
| chr4 | 144598972 | 144599022 |
| chr4 | 157281132 | 157281182 |
| chr4 | 161645228 | 161645278 |
| chr4 | 166237474 | 166237524 |
| chr4 | 171564258 | 171564308 |
| chr4 | 191071761 | 191071811 |
| chr5 | 6075254 | 6075304 |
| chr5 | 6075012 | 6075062 |
| chr5 | 6437024 | 6437074 |
| chr5 | 10171631 | 10171681 |
| chr5 | 25452937 | 25452987 |
| chr5 | 37802862 | 37802912 |
| chr5 | 38265439 | 38265489 |
| chr5 | 56464643 | 56464693 |
| chr5 | 78591185 | 78591235 |
| chr5 | 81327129 | 81327179 |
| chr5 | 103818213 | 103818263 |
| chr11 | 71889108 | 71889158 |
| chr10 | 14521697 | 14521747 |
| chr5 | 132010239 | 132010289 |
| chr5 | 143589416 | 143589466 |
| chr5 | 157379428 | 157379478 |
| chr5 | 163762160 | 163762210 |
| chr5 | 163761885 | 163761935 |
| chr5 | 169800405 | 169800455 |
| chr5 | 173790037 | 173790087 |
| chr5 | 177776936 | 177776986 |
| chr6 | 24846609 | 24846659 |
| chr6 | 27417204 | 27417254 |
| chr11 | 84450242 | 84450292 |
| chr6 | 49482828 | 49482878 |
| chr11 | 89010640 | 89010690 |
| chr6 | 82773885 | 82773935 |
| chr6 | 93542478 | 93542528 |
| chr6 | 133187616 | 133187666 |
| chr10 | 15808906 | 15808956 |
| chr11 | 105157151 | 105157201 |
| chr6 | 170573016 | 170573066 |
| chr6 | 170572746 | 170572796 |
| chr6 | 170573098 | 170573148 |
| chr7 | 764166 | 764216 |
| chr7 | 764361 | 764411 |
| chr7 | 1304506 | 1304556 |
| chr7 | 2499463 | 2499513 |
| chr7 | 2499279 | 2499329 |
| chr7 | 2782798 | 2782848 |
| chr7 | 4195740 | 4195790 |
| chr7 | 4363467 | 4363517 |
| chr7 | 22144728 | 22144778 |
| chr7 | 47271517 | 47271567 |
| chr7 | 47271430 | 47271480 |
| chr7 | 55588403 | 55588453 |
| chr7 | 99220910 | 99220960 |
| chr7 | 129353402 | 129353452 |
| chr7 | 138549900 | 138549950 |
| chr7 | 145539469 | 145539519 |
| chr11 | 121297297 | 121297347 |
| chr8 | 17139164 | 17139214 |
| chr8 | 17702176 | 17702226 |
| chr8 | 88865698 | 88865748 |
| chr11 | 128156948 | 128156998 |
| chr8 | 103212414 | 103212464 |
| chr8 | 103212500 | 103212550 |
| chr8 | 109971604 | 109971654 |
| chr8 | 140647303 | 140647353 |
| chr9 | 3469781 | 3469831 |
| chr9 | 12259100 | 12259150 |
| chr9 | 33161868 | 33161918 |
| chr11 | 133223176 | 133223226 |
| chr11 | 133222861 | 133222911 |
| chr9 | 74567171 | 74567221 |
| chr9 | 86380195 | 86380245 |
| chr9 | 93738245 | 93738295 |
| chr9 | 110920241 | 110920291 |
| chr9 | 133382001 | 133382051 |
| chr9 | 133383010 | 133383060 |
| chr10 | 22500800 | 22500850 |
| chr12 | 15801880 | 15801930 |
| chr12 | 27248979 | 27249029 |
| chr12 | 47592583 | 47592633 |
| chr12 | 84275159 | 84275209 |
| chr10 | 29416911 | 29416961 |
| chr12 | 88239050 | 88239100 |
| chr12 | 111249132 | 111249182 |
| chr12 | 112566569 | 112566619 |
| chr12 | 112913349 | 112913399 |
| chr12 | 113927157 | 113927207 |
| chr12 | 113926953 | 113927003 |
| chr12 | 115418466 | 115418516 |
| chr12 | 122348956 | 122349006 |
| chr13 | 70313039 | 70313089 |
| chr13 | 71014871 | 71014921 |
| chr13 | 85976622 | 85976672 |
| chr13 | 101221176 | 101221226 |
| chr13 | 101221110 | 101221160 |
| chr14 | 21259955 | 21260005 |
| chr14 | 56853950 | 56854000 |
| chr14 | 57949844 | 57949894 |
| chr14 | 72057569 | 72057619 |
| chr14 | 74616673 | 74616723 |
| chr14 | 89708039 | 89708089 |
| chr14 | 101217495 | 101217545 |
| chr14 | 102073153 | 102073203 |
| chr14 | 103096635 | 103096685 |
| chr15 | 25950421 | 25950471 |
| chr15 | 35413729 | 35413779 |
| chr10 | 69359082 | 69359132 |
| chr15 | 99815043 | 99815093 |
| chr16 | 16794897 | 16794947 |
| chr16 | 18602324 | 18602374 |
| chr16 | 51408954 | 51409004 |
| chr10 | 73899939 | 73899989 |
| chr16 | 75212195 | 75212245 |
| chr16 | 85517429 | 85517479 |
| chr16 | 86447566 | 86447616 |
| chr16 | 86596769 | 86596819 |
| chr16 | 88258867 | 88258917 |
| chr16 | 89513481 | 89513531 |
| chr17 | 540372 | 540422 |
| chr17 | 7969867 | 7969917 |
| chr17 | 16863762 | 16863812 |
| chr17 | 19176037 | 19176087 |
| chr17 | 36058731 | 36058781 |

Table 15DH. Repeat Category MLT2A2

| | | |
|---|---|---|
| chr17 | 51756645 | 51756695 |
| chr17 | 61506008 | 61506058 |
| chr17 | 68455881 | 68455931 |
| chr18 | 38016939 | 38016989 |
| chr18 | 38016466 | 38016516 |
| chr18 | 42958118 | 42958168 |
| chr10 | 98244194 | 98244244 |
| chr18 | 67484894 | 67484944 |
| chr10 | 106907327 | 106907377 |
| chr19 | 51405439 | 51405489 |
| chr19 | 51405283 | 51405333 |
| chr19 | 59443366 | 59443416 |
| chr1 | 3909422 | 3909472 |
| chr1 | 8906545 | 8906595 |
| chr1 | 9279370 | 9279420 |
| chr1 | 9297577 | 9297627 |
| chr1 | 12273663 | 12273713 |
| chr1 | 63073556 | 63073606 |
| chr1 | 88529129 | 88529179 |
| chr1 | 96184470 | 96184520 |
| chr10 | 6825607 | 6825657 |
| chr10 | 6825186 | 6825236 |
| chr10 | 125859633 | 125859683 |
| chr1 | 192561402 | 192561452 |
| chr10 | 131335709 | 131335759 |
| chr20 | 47842721 | 47842771 |
| chr21 | 20737501 | 20737551 |
| chr21 | 28595088 | 28595138 |
| chr21 | 29145010 | 29145060 |
| chr21 | 46615321 | 46615371 |
| chr22 | 15312379 | 15312429 |
| chrX | 4764063 | 4764113 |
| chrX | 6372101 | 6372151 |
| chrX | 17784709 | 17784759 |
| chrX | 19416489 | 19416539 |
| chrX | 88082370 | 88082420 |
| chrX | 101263683 | 101263733 |
| chrX | 114708023 | 114708073 |
| chrY | 3305969 | 3306019 |
| chr2 | 5087716 | 5087766 |
| chr2 | 6982850 | 6982900 |
| chr11 | 18195154 | 18195204 |
| chr11 | 18194906 | 18194956 |
| chr11 | 18440395 | 18440445 |
| chr2 | 37866238 | 37866288 |
| chr11 | 20553729 | 20553779 |
| chr2 | 56825740 | 56825790 |
| chr11 | 27753445 | 27753495 |
| chr2 | 103118135 | 103118185 |
| chr2 | 133559628 | 133559678 |
| chr11 | 33932843 | 33932893 |
| chr2 | 170920350 | 170920400 |
| chr2 | 199607850 | 199607900 |
| chr2 | 203969901 | 203969951 |
| chr2 | 224369830 | 224369880 |
| chr2 | 224937948 | 224937998 |
| chr3 | 14998439 | 14998489 |
| chr3 | 34725721 | 34725771 |
| chr3 | 35621701 | 35621751 |
| chr3 | 68470120 | 68470170 |
| chr3 | 74516329 | 74516379 |
| chr3 | 77645541 | 77645591 |
| chr3 | 105605117 | 105605167 |
| chr3 | 106151957 | 106152007 |
| chr3 | 109781633 | 109781683 |
| chr3 | 169820168 | 169820218 |
| chr3 | 194057014 | 194057064 |
| chr4 | 32868244 | 32868294 |
| chr4 | 34107951 | 34108001 |
| chr4 | 59153299 | 59153349 |
| chr4 | 89912622 | 89912672 |
| chr4 | 117018984 | 117019034 |
| chr4 | 132678146 | 132678196 |
| chr4 | 140941457 | 140941507 |
| chr4 | 161611356 | 161611406 |
| chr4 | 175466338 | 175466388 |
| chr5 | 2033420 | 2033470 |
| chr5 | 2278185 | 2278235 |
| chr5 | 6932896 | 6932946 |
| chr5 | 22684338 | 22684388 |
| chr5 | 26747254 | 26747304 |
| chr11 | 69718886 | 69718936 |
| chr5 | 44985597 | 44985647 |
| chr5 | 64798423 | 64798473 |
| chr5 | 121540580 | 121540630 |
| chr5 | 122382726 | 122382776 |
| chr5 | 162323487 | 162323537 |
| chr6 | 11889422 | 11889472 |
| chr6 | 50961577 | 50961627 |
| chr11 | 90082072 | 90082122 |
| chr6 | 103252161 | 103252211 |
| chr6 | 105710211 | 105710261 |
| chr11 | 97450060 | 97450110 |
| chr6 | 146569176 | 146569226 |
| chr6 | 148129775 | 148129825 |
| chr11 | 104666946 | 104666996 |
| chr7 | 69914072 | 69914122 |
| chr7 | 69916233 | 69916283 |
| chr7 | 104622716 | 104622766 |
| chr7 | 110266516 | 110266566 |
| chr7 | 115481261 | 115481311 |
| chr7 | 135292474 | 135292524 |
| chr8 | 2131193 | 2131243 |
| chr8 | 14347269 | 14347319 |
| chr8 | 40224296 | 40224346 |
| chr9 | 12371070 | 12371120 |
| chr9 | 20815899 | 20815949 |
| chr9 | 20815816 | 20815866 |
| chr9 | 113485125 | 113485175 |
| chr12 | 3744503 | 3744553 |
| chr12 | 54946744 | 54946794 |
| chr12 | 56236684 | 56236734 |
| chr12 | 59251633 | 59251683 |
| chr12 | 104506203 | 104506253 |
| chr12 | 106314005 | 106314055 |
| chr12 | 111435179 | 111435229 |
| chr12 | 125835508 | 125835558 |
| chr10 | 34899259 | 34899309 |
| chr10 | 42034027 | 42034077 |
| chr13 | 52661975 | 52662025 |
| chr13 | 103856039 | 103856089 |
| chr13 | 103855841 | 103855891 |
| chr14 | 59622519 | 59622569 |
| chr14 | 60513579 | 60513629 |
| chr14 | 69842721 | 69842771 |
| chr14 | 93626408 | 93626458 |
| chr14 | 100353991 | 100354041 |
| chr14 | 102525042 | 102525092 |
| chr14 | 104932537 | 104932587 |
| chr15 | 22839882 | 22839932 |
| chr15 | 37820735 | 37820785 |
| chr15 | 81714328 | 81714378 |
| chr16 | 13494592 | 13494642 |
| chr16 | 89134294 | 89134344 |
| chr17 | 4230259 | 4230309 |
| chr17 | 21179866 | 21179916 |

Table 15DI. Repeat Category MLT2B1

| | | |
|---|---|---|
| chr17 | 64813207 | 64813257 |
| chr18 | 869566 | 869616 |
| chr18 | 12891931 | 12891981 |
| chr18 | 16993075 | 16993125 |
| chr18 | 46624091 | 46624141 |
| chr18 | 68910954 | 68911004 |
| chr18 | 71015702 | 71015752 |
| chr18 | 74481918 | 74481968 |
| chr19 | 14296119 | 14296169 |
| chr19 | 18793111 | 18793161 |
| chr19 | 45408107 | 45408157 |
| chr19 | 59274379 | 59274429 |
| chr19 | 60674467 | 60674517 |
| chr1 | 9294515 | 9294565 |
| chr1 | 11154981 | 11155031 |
| chr1 | 23626682 | 23626732 |
| chr1 | 39483892 | 39483942 |
| chr1 | 49242874 | 49242924 |
| chr1 | 119973468 | 119973518 |
| chr10 | 7537343 | 7537393 |
| chr1 | 240975151 | 240975201 |
| chr1 | 241508439 | 241508489 |
| chr1 | 246005048 | 246005098 |
| chr20 | 2154318 | 2154368 |
| chr20 | 11372729 | 11372779 |
| chr20 | 38742153 | 38742203 |
| chr20 | 46773608 | 46773658 |
| chr21 | 25200111 | 25200161 |
| chr21 | 33118597 | 33118647 |
| chr21 | 43747724 | 43747774 |
| chr21 | 43747504 | 43747554 |
| chr11 | 1601856 | 1601906 |
| chr22 | 34302719 | 34302769 |
| chr22 | 39381226 | 39381276 |
| chr22 | 42710174 | 42710224 |
| chr22 | 45935208 | 45935258 |
| chr22 | 46239906 | 46239956 |
| chr22 | 46239663 | 46239713 |
| chr22 | 47538539 | 47538589 |
| chr22 | 47913364 | 47913414 |
| chrX | 29879581 | 29879631 |
| chrX | 29905010 | 29905060 |
| chrX | 30730952 | 30731002 |
| chrX | 117591018 | 117591068 |
| chrY | 6144298 | 6144348 |
| chr2 | 22843654 | 22843704 |
| chr2 | 43092909 | 43092959 |
| chr2 | 62475076 | 62475126 |
| chr2 | 78874346 | 78874396 |
| chr2 | 79584169 | 79584219 |
| chr2 | 137666784 | 137666834 |
| chr2 | 155679366 | 155679416 |
| chr2 | 175333644 | 175333694 |
| chr2 | 203754217 | 203754267 |
| chr2 | 206946511 | 206946561 |
| chr2 | 237602498 | 237602548 |
| chr2 | 241947266 | 241947316 |
| chr3 | 15149900 | 15149950 |
| chr3 | 16362141 | 16362191 |
| chr11 | 49070741 | 49070791 |
| chr3 | 71859392 | 71859442 |
| chr11 | 58237769 | 58237819 |
| chr3 | 124459135 | 124459185 |
| chr3 | 124458995 | 124459045 |
| chr3 | 156037804 | 156037854 |
| chr3 | 174491165 | 174491215 |
| chr11 | 63109115 | 63109165 |
| chr4 | 8743364 | 8743414 |
| chr4 | 36868320 | 36868370 |
| chr4 | 39562913 | 39562963 |
| chr4 | 42159278 | 42159328 |
| chr4 | 45511568 | 45511618 |
| chr11 | 65250440 | 65250490 |
| chr4 | 91637694 | 91637744 |
| chr4 | 110633745 | 110633795 |
| chr4 | 111594382 | 111594432 |
| chr4 | 127195479 | 127195529 |
| chr4 | 166297456 | 166297506 |
| chr10 | 13816303 | 13816353 |
| chr10 | 13816183 | 13816233 |
| chr5 | 8807918 | 8807968 |
| chr5 | 21573722 | 21573772 |
| chr5 | 25938550 | 25938600 |
| chr5 | 39712614 | 39712664 |
| chr5 | 61431233 | 61431283 |
| chr5 | 86612830 | 86612880 |
| chr5 | 86612658 | 86612708 |
| chr11 | 72688134 | 72688184 |
| chr10 | 14745869 | 14745919 |
| chr5 | 172981585 | 172981635 |
| chr6 | 10966242 | 10966292 |
| chr11 | 80242000 | 80242050 |
| chr6 | 28705039 | 28705089 |
| chr11 | 89172265 | 89172315 |
| chr6 | 71296452 | 71296502 |
| chr6 | 105747608 | 105747658 |
| chr6 | 124874258 | 124874308 |
| chr6 | 131861203 | 131861253 |
| chr6 | 154870278 | 154870328 |
| chr6 | 169731254 | 169731304 |
| chr7 | 3976774 | 3976824 |
| chr7 | 4437466 | 4437516 |
| chr7 | 8619853 | 8619903 |
| chr7 | 15486372 | 15486422 |
| chr7 | 16314666 | 16314716 |
| chr7 | 16314370 | 16314420 |
| chr7 | 37398535 | 37398585 |
| chr7 | 46434126 | 46434176 |
| chr7 | 64486710 | 64486760 |
| chr7 | 97275229 | 97275279 |
| chr7 | 122760060 | 122760110 |
| chr7 | 126106333 | 126106383 |
| chr7 | 134818844 | 134818894 |
| chr7 | 155240895 | 155240945 |
| chr7 | 156341430 | 156341480 |
| chr7 | 157819504 | 157819554 |
| chr8 | 222053 | 222103 |
| chr8 | 12446641 | 12446691 |
| chr8 | 23508115 | 23508165 |
| chr8 | 26975212 | 26975262 |
| chr8 | 52607316 | 52607366 |
| chr8 | 83052881 | 83052931 |
| chr8 | 103450798 | 103450848 |
| chr8 | 137520868 | 137520918 |
| chr9 | 15338085 | 15338135 |
| chr9 | 42639813 | 42639863 |
| chr9 | 76603008 | 76603058 |
| chr9 | 126685267 | 126685317 |
| chr9 | 132082816 | 132082866 |
| chr9 | 132625264 | 132625314 |
| chr9 | 132894599 | 132894649 |
| chr9 | 133596915 | 133596965 |
| chr9 | 133711537 | 133711587 |
| chr12 | 13420931 | 13420981 |
| chr12 | 13420842 | 13420892 |
| chr12 | 15213662 | 15213712 |
| chr12 | 15213390 | 15213440 |
| chr12 | 54160277 | 54160327 |
| chr12 | 56358586 | 56358636 |
| chr12 | 88184808 | 88184858 |
| chr12 | 89198972 | 89199022 |
| chr12 | 100586100 | 100586150 |
| chr12 | 127686575 | 127686625 |
| chr12 | 128413797 | 128413847 |
| chr10 | 36009656 | 36009706 |
| chr13 | 25407090 | 25407140 |
| chr13 | 42788680 | 42788730 |
| chr13 | 58036223 | 58036273 |
| chr14 | 19013552 | 19013602 |
| chr14 | 19595168 | 19595218 |
| chr14 | 36898760 | 36898810 |
| chr14 | 57623612 | 57623662 |
| chr14 | 72323518 | 72323568 |
| chr14 | 81120912 | 81120962 |
| chr10 | 57552657 | 57552707 |
| chr15 | 21261738 | 21261788 |
| chr15 | 91737448 | 91737498 |
| chr15 | 92194416 | 92194466 |
| chr16 | 11247497 | 11247547 |
| chr16 | 12114668 | 12114718 |
| chr16 | 19805588 | 19805638 |
| chr16 | 20900756 | 20900806 |
| chr16 | 49584112 | 49584162 |
| chr16 | 50630494 | 50630544 |
| chr16 | 54982509 | 54982559 |
| chr16 | 84011623 | 84011673 |
| chr16 | 84011832 | 84011882 |
| chr17 | 15114820 | 15114870 |
| chr17 | 26902719 | 26902769 |

Table 15DJ. Repeat Category MLT2B2

| | | |
|---|---|---|
| chr17 | 42324152 | 42324202 |
| chr17 | 51513647 | 51513697 |
| chr18 | 2472063 | 2472113 |
| chr18 | 47025108 | 47025158 |
| chr19 | 1842578 | 1842628 |
| chr19 | 5913243 | 5913293 |
| chr1 | 25520823 | 25520873 |
| chr1 | 40216538 | 40216588 |
| chr1 | 103974751 | 103974801 |
| chr1 | 164040876 | 164040926 |
| chr1 | 183918698 | 183918748 |
| chr1 | 192085481 | 192085531 |
| chr10 | 131824919 | 131824969 |
| chr10 | 132134256 | 132134306 |
| chr1 | 244499146 | 244499196 |
| chr20 | 4985317 | 4985367 |
| chr20 | 22127357 | 22127407 |
| chr20 | 40945275 | 40945325 |
| chr20 | 55952755 | 55952805 |
| chr21 | 18211675 | 18211725 |
| chr21 | 18211411 | 18211461 |
| chr21 | 22863933 | 22863983 |
| chr22 | 47519185 | 47519235 |
| chrX | 17859254 | 17859304 |
| chrX | 43622446 | 43622496 |
| chrX | 44176902 | 44176952 |
| chrX | 98507925 | 98507975 |
| chrX | 110257160 | 110257210 |
| chrY | 7125685 | 7125735 |
| chr2 | 8444248 | 8444298 |
| chr2 | 30501804 | 30501854 |
| chr2 | 53958434 | 53958484 |
| chr2 | 116810202 | 116810252 |
| chr2 | 153280475 | 153280525 |
| chr2 | 186283894 | 186283944 |
| chr2 | 204295646 | 204295696 |
| chr2 | 228727285 | 228727335 |
| chr2 | 240711975 | 240712025 |
| chr3 | 53270082 | 53270132 |
| chr3 | 108288719 | 108288769 |
| chr3 | 108288519 | 108288569 |
| chr3 | 128217716 | 128217766 |
| chr3 | 145865320 | 145865370 |
| chr3 | 185450976 | 185451026 |
| chr4 | 5692963 | 5693013 |
| chr11 | 65213837 | 65213887 |
| chr11 | 65214358 | 65214408 |
| chr4 | 153633265 | 153633315 |
| chr4 | 184964214 | 184964264 |
| chr4 | 184964334 | 184964384 |
| chr4 | 184965109 | 184965159 |
| chr4 | 185184983 | 185185033 |
| chr4 | 188898341 | 188898391 |
| chr5 | 14169093 | 14169143 |
| chr5 | 76757706 | 76757756 |
| chr5 | 130647790 | 130647840 |
| chr5 | 131229208 | 131229258 |
| chr5 | 131772240 | 131772290 |
| chr5 | 176388024 | 176388074 |
| chr6 | 21096792 | 21096842 |
| chr11 | 89124528 | 89124578 |
| chr6 | 137411784 | 137411834 |
| chr7 | 47366209 | 47366259 |
| chr7 | 47457236 | 47457286 |
| chr11 | 114462889 | 114462939 |
| chr10 | 17433699 | 17433749 |
| chr8 | 2760276 | 2760326 |
| chr11 | 122277054 | 122277104 |
| chr8 | 93178573 | 93178623 |
| chr8 | 142540071 | 142540121 |
| chr8 | 142971896 | 142971946 |
| chr9 | 38880245 | 38880295 |
| chr9 | 73935023 | 73935073 |
| chr9 | 105476092 | 105476142 |
| chr9 | 105476006 | 105476056 |
| chr12 | 82119256 | 82119306 |
| chr12 | 103065833 | 103065883 |
| chr12 | 125529778 | 125529828 |
| chr10 | 44089462 | 44089512 |
| chr10 | 44089692 | 44089742 |
| chr14 | 85015790 | 85015840 |
| chr14 | 103100312 | 103100362 |
| chr16 | 10300759 | 10300809 |
| chr16 | 16906758 | 16906808 |
| chr16 | 27489357 | 27489407 |
| chr16 | 60377051 | 60377101 |
| chr16 | 68431780 | 68431830 |
| chr17 | 13923277 | 13923327 |
| chr17 | 26927387 | 26927437 |
| chr17 | 27276932 | 27276982 |
| chr10 | 3737554 | 3737604 |

Table 15DK. Repeat Category MLT2B3

| | | |
|---|---|---|
| chr17 | 49888862 | 49888912 |
| chr10 | 95264677 | 95264727 |
| chr18 | 22041995 | 22042045 |
| chr18 | 37083225 | 37083275 |
| chr18 | 42490825 | 42490875 |
| chr10 | 97590744 | 97590794 |
| chr18 | 66218240 | 66218290 |
| chr18 | 68944895 | 68944945 |
| chr19 | 47728580 | 47728630 |
| chr19 | 47729535 | 47729585 |
| chr19 | 53255201 | 53255251 |
| chr10 | 112854925 | 112854975 |
| chr19 | 59648218 | 59648268 |
| chr1 | 13155265 | 13155315 |
| chr1 | 19095012 | 19095062 |
| chr1 | 23845639 | 23845689 |
| chr1 | 31977074 | 31977124 |
| chr1 | 35144722 | 35144772 |
| chr1 | 36134155 | 36134205 |
| chr1 | 49757364 | 49757414 |
| chr1 | 91068049 | 91068099 |
| chr1 | 91068263 | 91068313 |
| chr1 | 91068479 | 91068529 |
| chr1 | 91068828 | 91068878 |
| chr1 | 108845441 | 108845491 |
| chr1 | 118919477 | 118919527 |
| chr1 | 140876147 | 140876197 |
| chr10 | 127045012 | 127045062 |
| chr1 | 212251208 | 212251258 |
| chr1 | 220036870 | 220036920 |
| chr1 | 221999851 | 221999901 |
| chr21 | 26101449 | 26101499 |
| chr22 | 39303942 | 39303992 |
| chrX | 7545724 | 7545774 |
| chrX | 24901828 | 24901878 |
| chrX | 79745570 | 79745620 |
| chrX | 81092602 | 81092652 |
| chrX | 88483053 | 88483103 |
| chrX | 91309953 | 91310003 |
| chrX | 92919015 | 92919065 |
| chrX | 101263683 | 101263733 |
| chrX | 137506868 | 137506918 |
| chr2 | 924683 | 924733 |
| chr2 | 15066592 | 15066642 |
| chr2 | 77632501 | 77632551 |
| chr2 | 123251438 | 123251488 |
| chr2 | 144889273 | 144889323 |
| chr2 | 166290295 | 166290345 |
| chr2 | 168884380 | 168884430 |
| chr3 | 42586294 | 42586344 |
| chr11 | 54806468 | 54806518 |
| chr3 | 107690373 | 107690423 |
| chr3 | 110614107 | 110614157 |
| chr3 | 141881935 | 141881985 |
| chr3 | 154031575 | 154031625 |
| chr3 | 166336650 | 166336700 |
| chr3 | 192959356 | 192959406 |
| chr4 | 6375852 | 6375902 |
| chr11 | 64045589 | 64045639 |
| chr4 | 13211129 | 13211179 |
| chr4 | 61366061 | 61366111 |
| chr4 | 95536618 | 95536668 |
| chr4 | 129845885 | 129845935 |
| chr4 | 142706241 | 142706291 |
| chr4 | 166625966 | 166626016 |
| chr4 | 184645832 | 184645882 |
| chr4 | 186824022 | 186824072 |
| chr5 | 39494380 | 39494430 |
| chr5 | 45884691 | 45884741 |
| chr5 | 52695082 | 52695132 |
| chr11 | 70609329 | 70609379 |
| chr5 | 89693137 | 89693187 |
| chr5 | 100625253 | 100625303 |
| chr6 | 10101594 | 10101644 |
| chr6 | 21762273 | 21762323 |
| chr6 | 38123665 | 38123715 |
| chr6 | 68839330 | 68839380 |
| chr6 | 80963036 | 80963086 |
| chr6 | 116131504 | 116131554 |
| chr6 | 122458270 | 122458320 |
| chr6 | 137411784 | 137411834 |
| chr11 | 99456629 | 99456679 |
| chr7 | 1304506 | 1304556 |
| chr7 | 19029198 | 19029248 |
| chr7 | 19030908 | 19030958 |
| chr7 | 42251464 | 42251514 |
| chr7 | 53621070 | 53621120 |
| chr7 | 53621388 | 53621438 |
| chr7 | 109931413 | 109931463 |
| chr7 | 110694806 | 110694856 |
| chr7 | 146878109 | 146878159 |
| chr7 | 149081470 | 149081520 |
| chr7 | 153192272 | 153192322 |
| chr7 | 158362589 | 158362639 |
| chr8 | 6059943 | 6059993 |
| chr8 | 13012225 | 13012275 |
| chr8 | 14814171 | 14814221 |
| chr8 | 30115657 | 30115707 |
| chr8 | 30115580 | 30115630 |
| chr8 | 33100860 | 33100910 |
| chr8 | 38099130 | 38099180 |
| chr8 | 40224296 | 40224346 |
| chr8 | 103101496 | 103101546 |
| chr8 | 122699189 | 122699239 |
| chr8 | 127765825 | 127765875 |
| chr8 | 141051202 | 141051252 |
| chr9 | 14832661 | 14832711 |
| chr9 | 28426844 | 28426894 |
| chr9 | 33189448 | 33189498 |
| chr9 | 36552123 | 36552173 |
| chr9 | 36552755 | 36552805 |
| chr9 | 85511805 | 85511855 |
| chr11 | 134289347 | 134289397 |
| chr9 | 126998275 | 126998325 |
| chr9 | 126998807 | 126998857 |
| chr9 | 132024899 | 132024949 |
| chr9 | 132141966 | 132142016 |
| chr9 | 132317874 | 132317924 |
| chr12 | 10469448 | 10469498 |
| chr12 | 22979813 | 22979863 |
| chr13 | 82836075 | 82836125 |
| chr14 | 33286669 | 33286719 |
| chr15 | 21774081 | 21774131 |
| chr15 | 62393346 | 62393396 |
| chr16 | 799691 | 799741 |
| chr16 | 19258937 | 19258987 |
| chr16 | 57302318 | 57302368 |
| chr16 | 58064548 | 58064598 |
| chr16 | 73715126 | 73715176 |
| chr17 | 279849 | 279899 |
| chr17 | 8157601 | 8157651 |
| chr17 | 28244775 | 28244825 |

Table 15DL. Repeat Category MLT2B4

| | | |
|---|---|---|
| chr17 | 76057738 | 76057788 |
| chr17 | 76114760 | 76114810 |
| chr17 | 76116883 | 76116933 |
| chr18 | 3619827 | 3619877 |
| chr18 | 75730292 | 75730342 |
| chr19 | 54705615 | 54705665 |
| chr10 | 118349143 | 118349193 |
| chr1 | 21217328 | 21217378 |
| chr1 | 62253220 | 62253270 |
| chr1 | 82052362 | 82052412 |
| chr10 | 6800392 | 6800442 |
| chr1 | 152351055 | 152351105 |
| chr1 | 162047623 | 162047673 |
| chr1 | 175962612 | 175962662 |
| chr1 | 190665709 | 190665759 |
| chr1 | 242419138 | 242419188 |
| chr20 | 3748977 | 3749027 |
| chr20 | 36103118 | 36103168 |
| chr20 | 38390453 | 38390503 |
| chr20 | 47209101 | 47209151 |
| chr20 | 47212140 | 47212190 |
| chr20 | 47211823 | 47211873 |
| chr20 | 47435851 | 47435901 |
| chr20 | 47785934 | 47785984 |
| chr20 | 47787367 | 47787417 |
| chr20 | 47818208 | 47818258 |
| chr21 | 17922576 | 17922626 |
| chr21 | 22016893 | 22016943 |
| chr21 | 31741048 | 31741098 |
| chr21 | 40782219 | 40782269 |
| chr22 | 35432316 | 35432366 |
| chr22 | 39935942 | 39935992 |
| chr22 | 41077304 | 41077354 |
| chr22 | 41153900 | 41153950 |
| chr22 | 41155029 | 41155079 |
| chr22 | 41326511 | 41326561 |
| chr22 | 43633345 | 43633395 |
| chr22 | 44532882 | 44532932 |
| chrX | 3066239 | 3066289 |
| chrX | 3166567 | 3166617 |
| chrX | 22501900 | 22501950 |
| chrX | 33526748 | 33526798 |
| chrX | 105389184 | 105389234 |
| chrX | 111031038 | 111031088 |
| chrX | 116934192 | 116934242 |
| chrX | 132790269 | 132790319 |
| chrX | 143266890 | 143266940 |
| chr2 | 10591259 | 10591309 |
| chr2 | 10592453 | 10592503 |
| chr2 | 13743753 | 13743803 |
| chr2 | 15246971 | 15247021 |
| chr2 | 32519841 | 32519891 |
| chr2 | 47480090 | 47480140 |
| chr2 | 55955226 | 55955276 |
| chr2 | 72599467 | 72599517 |
| chr2 | 112160061 | 112160111 |
| chr11 | 33932843 | 33932893 |
| chr2 | 147000420 | 147000470 |
| chr2 | 157253723 | 157253773 |
| chr2 | 164844199 | 164844249 |
| chr2 | 188936364 | 188936414 |
| chr2 | 225686623 | 225686673 |
| chr2 | 241364100 | 241364150 |
| chr2 | 243279464 | 243279514 |
| chr11 | 47416833 | 47416883 |
| chr3 | 23289221 | 23289271 |
| chr3 | 27557442 | 27557492 |
| chr3 | 35492659 | 35492709 |
| chr3 | 44435106 | 44435156 |
| chr3 | 50997473 | 50997523 |
| chr11 | 57543049 | 57543099 |
| chr3 | 85318069 | 85318119 |
| chr10 | 12792749 | 12792799 |
| chr3 | 171420074 | 171420124 |
| chr3 | 176609269 | 176609319 |
| chr3 | 178342233 | 178342283 |
| chr3 | 190403197 | 190403247 |
| chr3 | 196995464 | 196995514 |
| chr3 | 197057427 | 197057477 |
| chr3 | 198694503 | 198694553 |
| chr4 | 1289256 | 1289306 |
| chr4 | 3746624 | 3746674 |
| chr4 | 25706985 | 25707035 |
| chr4 | 36361613 | 36361663 |
| chr4 | 46066583 | 46066633 |
| chr4 | 55291213 | 55291263 |
| chr4 | 77238673 | 77238723 |
| chr4 | 116308807 | 116308857 |
| chr4 | 185942047 | 185942097 |
| chr4 | 189810150 | 189810200 |
| chr4 | 191279911 | 191279961 |
| chr5 | 1650636 | 1650686 |
| chr5 | 1674289 | 1674339 |
| chr5 | 1676869 | 1676919 |
| chr5 | 3083945 | 3083995 |
| chr5 | 25462370 | 25462420 |
| chr5 | 26271413 | 26271463 |
| chr5 | 26513753 | 26513803 |
| chr5 | 29831786 | 29831836 |
| chr5 | 30475097 | 30475147 |
| chr5 | 43295268 | 43295318 |
| chr5 | 76167067 | 76167117 |
| chr6 | 11426770 | 11426820 |
| chr6 | 11426577 | 11426627 |
| chr6 | 52404923 | 52404973 |
| chr6 | 52404817 | 52404867 |
| chr6 | 76257273 | 76257323 |
| chr6 | 150407323 | 150407373 |
| chr6 | 158814989 | 158815039 |
| chr6 | 166014491 | 166014541 |
| chr6 | 166881545 | 166881595 |
| chr6 | 166881337 | 166881387 |
| chr6 | 170021365 | 170021415 |
| chr7 | 2202097 | 2202147 |
| chr11 | 113272824 | 113272874 |
| chr11 | 113424814 | 113424864 |
| chr7 | 71803805 | 71803855 |
| chr7 | 92643844 | 92643894 |
| chr7 | 97753959 | 97754009 |
| chr7 | 98528803 | 98528853 |
| chr7 | 106529957 | 106530007 |
| chr7 | 122760060 | 122760110 |
| chr7 | 146323510 | 146323560 |
| chr8 | 6144762 | 6144812 |
| chr8 | 10166911 | 10166961 |
| chr8 | 10435924 | 10435974 |
| chr8 | 25001794 | 25001844 |
| chr8 | 80272702 | 80272752 |
| chr8 | 83052881 | 83052931 |
| chr8 | 86221696 | 86221746 |
| chr8 | 103410341 | 103410391 |
| chr9 | 76833828 | 76833878 |
| chr9 | 85826471 | 85826521 |
| chr9 | 90786194 | 90786244 |
| chr9 | 95938036 | 95938086 |
| chr9 | 104862008 | 104862058 |
| chr9 | 112452670 | 112452720 |
| chr9 | 122764946 | 122764996 |
| chr9 | 132580615 | 132580665 |
| chr9 | 134472238 | 134472288 |
| chr9 | 134744747 | 134744797 |
| chr12 | 31688673 | 31688723 |
| chr12 | 47580608 | 47580658 |
| chr12 | 84515765 | 84515815 |
| chr12 | 88670035 | 88670085 |
| chr12 | 94981437 | 94981487 |
| chr12 | 128132968 | 128133018 |
| chr13 | 28122798 | 28122848 |
| chr13 | 109462661 | 109462711 |
| chr14 | 79478772 | 79478822 |
| chr14 | 81560030 | 81560080 |
| chr14 | 94637515 | 94637565 |
| chr16 | 3248889 | 3248939 |
| chr16 | 65166885 | 65166935 |
| chr16 | 74373896 | 74373946 |
| chr16 | 87615363 | 87615413 |
| chr17 | 29059554 | 29059604 |

Table 15DM. Repeat Category MLT2D

| | | |
|---|---|---|
| chr17 | 42892627 | 42892677 |
| chr17 | 72581475 | 72581525 |
| chr18 | 20271821 | 20271871 |
| chr18 | 26386186 | 26386236 |
| chr18 | 35294094 | 35294144 |
| chr18 | 69101334 | 69101384 |
| chr19 | 6508751 | 6508801 |
| chr19 | 38948511 | 38948561 |
| chr19 | 52619852 | 52619902 |
| chr19 | 59238897 | 59238947 |
| chr1 | 1792237 | 1792287 |
| chr1 | 2732525 | 2732575 |
| chr1 | 3891642 | 3891692 |
| chr1 | 5614386 | 5614436 |
| chr1 | 7106875 | 7106925 |
| chr1 | 7675012 | 7675062 |
| chr1 | 13374220 | 13374270 |
| chr1 | 15202424 | 15202474 |
| chr1 | 30646775 | 30646825 |
| chr1 | 64425593 | 64425643 |
| chr1 | 66660569 | 66660619 |
| chr1 | 82896747 | 82896797 |
| chr1 | 93434106 | 93434156 |
| chr1 | 100673040 | 100673090 |
| chr1 | 190665709 | 190665759 |
| chr1 | 226702304 | 226702354 |
| chr1 | 230815266 | 230815316 |
| chr10 | 131932323 | 131932373 |
| chr1 | 238027964 | 238028014 |
| chr1 | 244446259 | 244446309 |
| chr20 | 13668055 | 13668105 |
| chr20 | 17789508 | 17789558 |
| chr20 | 34412449 | 34412499 |
| chr20 | 35953103 | 35953153 |
| chr10 | 133682067 | 133682117 |
| chr20 | 36391236 | 36391286 |
| chr20 | 41754886 | 41754936 |
| chr20 | 55956411 | 55956461 |
| chr20 | 60658952 | 60659002 |
| chr11 | 249897 | 249947 |
| chr21 | 25959866 | 25959916 |
| chr21 | 44755927 | 44755977 |
| chr22 | 21274136 | 21274186 |
| chr22 | 23908895 | 23908945 |
| chr22 | 23910026 | 23910076 |
| chr22 | 44201421 | 44201471 |
| chr22 | 46242729 | 46242779 |
| chrX | 3066239 | 3066289 |
| chrX | 23495332 | 23495382 |
| chrX | 40216681 | 40216731 |
| chrX | 51816777 | 51816827 |
| chrX | 54640989 | 54641039 |
| chrX | 88476179 | 88476229 |
| chrX | 132294706 | 132294756 |
| chrX | 132432650 | 132432700 |
| chr2 | 2937024 | 2937074 |
| chr2 | 48354622 | 48354672 |
| chr2 | 75011422 | 75011472 |
| chr2 | 78359108 | 78359158 |
| chr2 | 103118135 | 103118185 |
| chr2 | 113005174 | 113005224 |
| chr2 | 113004961 | 113005011 |
| chr2 | 113005822 | 113005872 |
| chr11 | 37232568 | 37232618 |
| chr2 | 186420932 | 186420982 |
| chr2 | 231303416 | 231303466 |
| chr11 | 45364619 | 45364669 |
| chr2 | 243020202 | 243020252 |
| chr3 | 636641 | 636691 |
| chr3 | 3259320 | 3259370 |
| chr3 | 15304330 | 15304380 |
| chr3 | 15304177 | 15304227 |
| chr3 | 44168386 | 44168436 |
| chr3 | 57058513 | 57058563 |
| chr3 | 128580101 | 128580151 |
| chr3 | 128580276 | 128580326 |
| chr3 | 131539530 | 131539580 |
| chr3 | 136092810 | 136092860 |
| chr3 | 139961212 | 139961262 |
| chr3 | 191789601 | 191789651 |
| chr4 | 3775205 | 3775255 |
| chr4 | 6173853 | 6173903 |
| chr4 | 7213137 | 7213187 |
| chr4 | 7221949 | 7221999 |
| chr11 | 64704210 | 64704260 |
| chr4 | 42112098 | 42112148 |
| chr4 | 46918627 | 46918677 |
| chr4 | 89006727 | 89006777 |
| chr4 | 98350549 | 98350599 |
| chr4 | 99692300 | 99692350 |
| chr4 | 132131127 | 132131177 |
| chr11 | 67099473 | 67099523 |
| chr4 | 160408961 | 160409011 |
| chr4 | 185228517 | 185228567 |
| chr4 | 185229918 | 185229968 |
| chr4 | 186620823 | 186620873 |
| chr5 | 17989253 | 17989303 |
| chr5 | 22805974 | 22806024 |
| chr5 | 76360250 | 76360300 |
| chr5 | 95127318 | 95127368 |
| chr5 | 121895650 | 121895700 |
| chr5 | 141434767 | 141434817 |
| chr5 | 168028580 | 168028630 |
| chr5 | 170150488 | 170150538 |
| chr5 | 173936851 | 173936901 |
| chr6 | 19892444 | 19892494 |
| chr6 | 27815049 | 27815099 |
| chr6 | 39833183 | 39833233 |
| chr6 | 42654824 | 42654874 |
| chr10 | 15290849 | 15290899 |
| chr6 | 115011254 | 115011304 |
| chr6 | 147835252 | 147835302 |
| chr11 | 101258719 | 101258769 |
| chr6 | 158814989 | 158815039 |
| chr6 | 168228942 | 168228992 |
| chr6 | 168842830 | 168842880 |
| chr7 | 10645580 | 10645630 |
| chr7 | 12517848 | 12517898 |
| chr7 | 55217291 | 55217341 |
| chr7 | 69839805 | 69839855 |
| chr11 | 118353449 | 118353499 |
| chr7 | 130765665 | 130765715 |
| chr7 | 131615325 | 131615375 |
| chr7 | 134360358 | 134360408 |
| chr7 | 148487590 | 148487640 |
| chr7 | 150977859 | 150977909 |
| chr7 | 153386640 | 153386690 |
| chr8 | 9425976 | 9426026 |
| chr8 | 26301614 | 26301664 |
| chr8 | 42495133 | 42495183 |
| chr8 | 52623676 | 52623726 |
| chr8 | 139920986 | 139921036 |
| chr8 | 144352829 | 144352879 |
| chr9 | 16134469 | 16134519 |
| chr9 | 67714503 | 67714553 |
| chr9 | 94830443 | 94830493 |
| chr9 | 136062331 | 136062381 |
| chr12 | 7994516 | 7994566 |
| chr12 | 8857636 | 8857686 |
| chr12 | 11757176 | 11757226 |
| chr12 | 32510748 | 32510798 |
| chr12 | 54160277 | 54160327 |
| chr12 | 56776455 | 56776505 |
| chr12 | 74195049 | 74195099 |
| chr12 | 75400118 | 75400168 |
| chr12 | 97505100 | 97505150 |
| chr10 | 31252721 | 31252771 |
| chr13 | 54027032 | 54027082 |
| chr13 | 54033072 | 54033122 |
| chr13 | 105292923 | 105292973 |
| chr13 | 105292841 | 105292891 |
| chr13 | 112500214 | 112500264 |
| chr14 | 28160143 | 28160193 |
| chr10 | 49875236 | 49875286 |
| chr14 | 53935954 | 53936004 |
| chr14 | 62168059 | 62168109 |
| chr14 | 88125836 | 88125886 |
| chr14 | 88342508 | 88342558 |
| chr14 | 102204340 | 102204390 |
| chr14 | 102391141 | 102391191 |
| chr14 | 102750844 | 102750894 |
| chr10 | 60029085 | 60029135 |
| chr15 | 43621850 | 43621900 |
| chr15 | 53798580 | 53798630 |
| chr15 | 94022203 | 94022253 |
| chr10 | 70147677 | 70147727 |
| chr10 | 3024852 | 3024902 |
| chr16 | 52094190 | 52094240 |
| chr16 | 56425008 | 56425058 |
| chr16 | 84489047 | 84489097 |
| chr17 | 2726633 | 2726683 |
| chr17 | 5940066 | 5940116 |
| chr17 | 12501162 | 12501212 |
| chr17 | 26929269 | 26929319 |

Table 15DN. Repeat Category MSR1

| | | |
|---|---|---|
| chr19 | 7718302 | 7718352 |
| chr19 | 7734193 | 7734243 |
| chr19 | 40259425 | 40259475 |
| chr19 | 41039038 | 41039088 |
| chr19 | 41136985 | 41137035 |
| chr19 | 43926467 | 43926517 |
| chr19 | 44379455 | 44379505 |
| chr19 | 44379385 | 44379435 |
| chr19 | 44585311 | 44585361 |
| chr19 | 44585656 | 44585706 |
| chr19 | 44585913 | 44585963 |
| chr19 | 46009354 | 46009404 |
| chr19 | 46009572 | 46009622 |
| chr19 | 46332686 | 46332736 |
| chr19 | 46332881 | 46332931 |
| chr19 | 47090604 | 47090654 |
| chr19 | 47089980 | 47090030 |
| chr19 | 47098641 | 47098691 |
| chr19 | 47180762 | 47180812 |
| chr19 | 47261173 | 47261223 |
| chr19 | 47261422 | 47261472 |
| chr19 | 47261098 | 47261148 |
| chr19 | 47566101 | 47566151 |
| chr19 | 48610867 | 48610917 |
| chr19 | 48611065 | 48611115 |
| chr19 | 48611301 | 48611351 |
| chr19 | 48675115 | 48675165 |
| chr19 | 48693386 | 48693436 |
| chr19 | 48702733 | 48702783 |
| chr19 | 48821598 | 48821648 |
| chr19 | 48823304 | 48823354 |
| chr19 | 49973223 | 49973273 |
| chr19 | 50004302 | 50004352 |
| chr19 | 50007509 | 50007559 |
| chr19 | 50008464 | 50008514 |
| chr19 | 50069337 | 50069387 |
| chr19 | 50068960 | 50069010 |
| chr19 | 53586677 | 53586727 |
| chr19 | 53641833 | 53641883 |
| chr19 | 53645623 | 53645673 |
| chr19 | 53664906 | 53664956 |
| chr19 | 53794639 | 53794689 |
| chr19 | 53909623 | 53909673 |
| chr19 | 53954543 | 53954593 |
| chr19 | 54031956 | 54032006 |
| chr19 | 54032859 | 54032909 |
| chr19 | 54067777 | 54067827 |
| chr19 | 54091621 | 54091671 |
| chr19 | 54095191 | 54095241 |
| chr19 | 54095326 | 54095376 |
| chr19 | 54128856 | 54128906 |
| chr19 | 54128659 | 54128709 |
| chr19 | 54128716 | 54128766 |
| chr19 | 54180973 | 54181023 |
| chr19 | 54180791 | 54180841 |
| chr19 | 54189157 | 54189207 |
| chr19 | 54253689 | 54253739 |
| chr19 | 54259965 | 54260015 |
| chr19 | 54260071 | 54260121 |
| chr19 | 54320824 | 54320874 |
| chr19 | 54353062 | 54353112 |
| chr19 | 54363706 | 54363756 |
| chr19 | 54555046 | 54555096 |
| chr19 | 54557467 | 54557517 |
| chr19 | 54621154 | 54621204 |
| chr19 | 54631785 | 54631835 |
| chr19 | 54707872 | 54707922 |
| chr19 | 54815658 | 54815708 |
| chr19 | 54835803 | 54835853 |
| chr19 | 54836265 | 54836315 |
| chr19 | 54841283 | 54841333 |
| chr19 | 54861140 | 54861190 |
| chr19 | 54887260 | 54887310 |
| chr19 | 54977783 | 54977833 |
| chr19 | 54977525 | 54977575 |
| chr19 | 54994844 | 54994894 |
| chr19 | 55013853 | 55013903 |
| chr19 | 55646529 | 55646579 |
| chr19 | 55662622 | 55662672 |
| chr19 | 55820361 | 55820411 |
| chr19 | 55824307 | 55824357 |
| chr19 | 56022427 | 56022477 |
| chr19 | 56163740 | 56163790 |
| chr19 | 56194330 | 56194380 |
| chr19 | 56196216 | 56196266 |
| chr19 | 56196129 | 56196179 |
| chr19 | 56272488 | 56272538 |
| chr19 | 56274637 | 56274687 |
| chr19 | 56274540 | 56274590 |
| chr19 | 56532902 | 56532952 |
| chr19 | 56583200 | 56583250 |
| chr19 | 56585644 | 56585694 |
| chr19 | 56797010 | 56797060 |
| chr19 | 56884480 | 56884530 |
| chr19 | 56887532 | 56887582 |
| chr19 | 56887722 | 56887772 |
| chr19 | 56887796 | 56887846 |
| chr19 | 59063370 | 59063420 |
| chr19 | 59064702 | 59064752 |
| chr19 | 59137020 | 59137070 |
| chr19 | 59290874 | 59290924 |
| chr19 | 59310010 | 59310060 |
| chr19 | 59365023 | 59365073 |
| chr19 | 59618579 | 59618629 |
| chr19 | 60316982 | 60317032 |
| chr19 | 60340229 | 60340279 |
| chr19 | 60365007 | 60365057 |
| chr19 | 60427830 | 60427880 |
| chr19 | 60427702 | 60427752 |
| chr19 | 60505696 | 60505746 |
| chr19 | 60523332 | 60523382 |
| chr19 | 60565462 | 60565512 |
| chr19 | 60572607 | 60572657 |
| chr19 | 60577642 | 60577692 |
| chr19 | 60640878 | 60640928 |
| chr19 | 60640702 | 60640752 |
| chr19 | 60739403 | 60739453 |
| chr19 | 60851697 | 60851747 |

Table 15DO. Repeat Category MSTA

| | | |
|---|---|---|
| chr17 | 45888066 | 45888116 |
| chr17 | 55581142 | 55581192 |
| chr17 | 57371424 | 57371474 |
| chr10 | 86045933 | 86045983 |
| chr17 | 62577665 | 62577715 |
| chr17 | 64354869 | 64354919 |
| chr17 | 65298382 | 65298432 |
| chr17 | 67042860 | 67042910 |
| chr17 | 71324294 | 71324344 |
| chr17 | 73478286 | 73478336 |
| chr17 | 75584998 | 75585048 |
| chr10 | 92395332 | 92395382 |
| chr17 | 80912139 | 80912189 |
| chr17 | 80913287 | 80913337 |
| chr17 | 81101333 | 81101383 |
| chr17 | 81697356 | 81697406 |
| chr18 | 516583 | 516633 |
| chr18 | 4418917 | 4418967 |
| chr18 | 5631183 | 5631233 |
| chr18 | 8411910 | 8411960 |
| chr18 | 19097151 | 19097201 |
| chr18 | 27532763 | 27532813 |
| chr18 | 28036231 | 28036281 |
| chr18 | 28036017 | 28036067 |
| chr18 | 28388101 | 28388151 |
| chr10 | 5014356 | 5014406 |
| chr10 | 5013846 | 5013896 |
| chr18 | 35450240 | 35450290 |
| chr18 | 35498079 | 35498129 |
| chr18 | 43746242 | 43746292 |
| chr18 | 50113828 | 50113878 |
| chr18 | 53882989 | 53883039 |
| chr18 | 54596391 | 54596441 |
| chr18 | 55399822 | 55399872 |
| chr18 | 62284049 | 62284099 |
| chr18 | 67368054 | 67368104 |
| chr18 | 67466511 | 67466561 |
| chr18 | 68222456 | 68222506 |
| chr18 | 68935572 | 68935622 |
| chr18 | 70615032 | 70615082 |
| chr18 | 75838445 | 75838495 |
| chr19 | 4993783 | 4993833 |
| chr19 | 8998136 | 8998186 |
| chr19 | 9119370 | 9119420 |
| chr19 | 12608461 | 12608511 |
| chr19 | 14142639 | 14142689 |
| chr19 | 14774019 | 14774069 |
| chr19 | 14773946 | 14773996 |
| chr19 | 15033996 | 15034046 |
| chr19 | 19728690 | 19728740 |
| chr19 | 34438741 | 34438791 |
| chr10 | 5720358 | 5720408 |
| chr19 | 37514377 | 37514427 |
| chr19 | 39261074 | 39261124 |
| chr19 | 39462351 | 39462401 |
| chr19 | 40201046 | 40201096 |
| chr10 | 104650009 | 104650059 |
| chr19 | 44246012 | 44246062 |
| chr19 | 45303470 | 45303520 |
| chr19 | 45348294 | 45348344 |
| chr19 | 46902543 | 46902593 |
| chr10 | 105726634 | 105726684 |
| chr10 | 5888108 | 5888158 |
| chr19 | 47876848 | 47876898 |
| chr10 | 107234376 | 107234426 |
| chr10 | 111510822 | 111510872 |
| chr19 | 54661536 | 54661586 |
| chr19 | 57259576 | 57259626 |
| chr19 | 57259638 | 57259688 |
| chr19 | 57368812 | 57368862 |
| chr10 | 113412970 | 113413020 |
| chr19 | 60418163 | 60418213 |
| chr19 | 60977531 | 60977581 |
| chr19 | 61673912 | 61673962 |
| chr19 | 62853071 | 62853121 |
| chr19 | 63326067 | 63326117 |
| chr19 | 63325829 | 63325879 |
| chr10 | 115206836 | 115206886 |
| chr1 | 2524079 | 2524129 |
| chr1 | 3571643 | 3571693 |
| chr1 | 4827952 | 4828002 |
| chr1 | 5200128 | 5200178 |
| chr1 | 5529622 | 5529672 |
| chr1 | 7898773 | 7898823 |
| chr1 | 7961784 | 7961834 |
| chr1 | 12435248 | 12435298 |
| chr1 | 14345870 | 14345920 |
| chr10 | 118575158 | 118575208 |
| chr1 | 16858564 | 16858614 |
| chr1 | 17009439 | 17009489 |
| chr1 | 17153095 | 17153145 |
| chr1 | 18516242 | 18516292 |
| chr1 | 18539263 | 18539313 |
| chr10 | 118822737 | 118822787 |
| chr1 | 20230080 | 20230130 |
| chr10 | 119183648 | 119183698 |
| chr1 | 21616192 | 21616242 |
| chr1 | 24478767 | 24478817 |
| chr10 | 121467587 | 121467637 |
| chr1 | 40985577 | 40985627 |
| chr1 | 42985078 | 42985128 |
| chr1 | 62190833 | 62190883 |
| chr1 | 67103364 | 67103414 |
| chr1 | 68497279 | 68497329 |
| chr1 | 70297151 | 70297201 |
| chr1 | 72083574 | 72083624 |
| chr1 | 72138274 | 72138324 |
| chr1 | 79285858 | 79285908 |
| chr1 | 81017631 | 81017681 |
| chr1 | 82386403 | 82386453 |
| chr1 | 83622755 | 83622805 |
| chr1 | 85130152 | 85130202 |
| chr1 | 86233589 | 86233639 |
| chr1 | 92028569 | 92028619 |
| chr1 | 110884704 | 110884754 |
| chr10 | 126185829 | 126185879 |
| chr1 | 112628853 | 112628903 |
| chr1 | 152614436 | 152614486 |
| chr1 | 153203479 | 153203529 |
| chr1 | 153203869 | 153203919 |
| chr1 | 175867474 | 175867524 |
| chr1 | 193173421 | 193173471 |
| chr1 | 200359027 | 200359077 |
| chr1 | 201186284 | 201186334 |
| chr1 | 201186822 | 201186872 |
| chr1 | 204138933 | 204138983 |
| chr1 | 204489468 | 204489518 |
| chr1 | 206286596 | 206286646 |
| chr1 | 210173229 | 210173279 |
| chr1 | 212443612 | 212443662 |
| chr1 | 215706938 | 215706988 |
| chr1 | 216210168 | 216210218 |
| chr1 | 217975141 | 217975191 |
| chr1 | 219405209 | 219405259 |
| chr1 | 221776796 | 221776846 |
| chr1 | 227660466 | 227660516 |
| chr1 | 228014767 | 228014817 |
| chr1 | 230969101 | 230969151 |
| chr1 | 232786093 | 232786143 |
| chr10 | 131804646 | 131804696 |
| chr1 | 236724634 | 236724684 |
| chr1 | 238449796 | 238449846 |
| chr1 | 239153469 | 239153519 |
| chr1 | 239161866 | 239161916 |
| chr1 | 242455444 | 242455494 |
| chr1 | 243569426 | 243569476 |
| chr20 | 1599328 | 1599378 |
| chr20 | 9587813 | 9587863 |
| chr20 | 10419827 | 10419877 |
| chr20 | 11741957 | 11742007 |
| chr20 | 12564601 | 12564651 |
| chr20 | 25214568 | 25214618 |
| chr20 | 25214677 | 25214727 |
| chr10 | 133507886 | 133507936 |
| chr20 | 33443400 | 33443450 |
| chr20 | 39407139 | 39407189 |
| chr20 | 39413065 | 39413115 |
| chr20 | 43062274 | 43062324 |
| chr20 | 46064724 | 46064774 |
| chr20 | 46064837 | 46064887 |
| chr20 | 46065723 | 46065773 |
| chr20 | 46159502 | 46159552 |
| chr20 | 47842721 | 47842771 |
| chr20 | 51560862 | 51560912 |
| chr20 | 52851641 | 52851691 |
| chr20 | 55962954 | 55963004 |
| chr20 | 56756651 | 56756701 |
| chr20 | 61370607 | 61370657 |
| chr20 | 63121198 | 63121248 |
| chr21 | 14152879 | 14152929 |
| chr21 | 17719211 | 17719261 |
| chr21 | 17997455 | 17997505 |
| chr21 | 20185894 | 20185944 |
| chr21 | 23113678 | 23113728 |
| chr21 | 23785087 | 23785137 |
| chr21 | 27012980 | 27013030 |
| chr21 | 27118714 | 27118764 |
| chr21 | 29292769 | 29292819 |
| chr21 | 30780124 | 30780174 |
| chr21 | 30779987 | 30780037 |
| chr21 | 33469512 | 33469562 |
| chr21 | 34986548 | 34986598 |
| chr21 | 36372195 | 36372245 |
| chr21 | 39043469 | 39043519 |
| chr21 | 39043790 | 39043840 |
| chr21 | 39410826 | 39410876 |
| chr21 | 39616467 | 39616517 |
| chr21 | 39872516 | 39872566 |
| chr21 | 41049265 | 41049315 |
| chr21 | 41913997 | 41914047 |
| chr21 | 42989815 | 42989865 |
| chr21 | 44937529 | 44937579 |
| chr21 | 44948841 | 44948891 |
| chr21 | 44954095 | 44954145 |
| chr21 | 45573639 | 45573689 |
| chr21 | 45573785 | 45573835 |
| chr21 | 45873464 | 45873514 |
| chr21 | 46239884 | 46239934 |
| chr22 | 16172293 | 16172343 |
| chr22 | 16370831 | 16370881 |
| chr22 | 18030983 | 18031033 |
| chr11 | 1363113 | 1363163 |
| chr22 | 24135046 | 24135096 |
| chr22 | 26348850 | 26348900 |
| chr22 | 27260849 | 27260899 |
| chr22 | 32080385 | 32080435 |
| chr22 | 32081435 | 32081485 |
| chr22 | 40186292 | 40186342 |
| chr22 | 41150166 | 41150216 |
| chr22 | 41408584 | 41408634 |
| chr22 | 43231655 | 43231705 |
| chr10 | 10128867 | 10128917 |
| chr22 | 43790731 | 43790781 |
| chr22 | 45585874 | 45585924 |
| chr22 | 45648763 | 45648813 |
| chr11 | 3177908 | 3177958 |
| chr22 | 47701737 | 47701787 |
| chr22 | 48270471 | 48270521 |
| chr22 | 48519005 | 48519055 |
| chrX | 2684622 | 2684672 |
| chrX | 3987288 | 3987338 |
| chrX | 11654542 | 11654592 |
| chrX | 15873075 | 15873125 |
| chrX | 17839781 | 17839831 |
| chrX | 23808855 | 23808905 |
| chrX | 23860859 | 23860909 |
| chrX | 26252159 | 26252209 |
| chrX | 34622260 | 34622310 |
| chrX | 38589527 | 38589577 |
| chrX | 45118395 | 45118445 |
| chrX | 46155942 | 46155992 |
| chrX | 46263953 | 46264003 |
| chrX | 52586295 | 52586345 |
| chrX | 55875396 | 55875446 |
| chrX | 74927522 | 74927572 |
| chrX | 76534776 | 76534826 |
| chrX | 87673683 | 87673733 |
| chrX | 88975494 | 88975544 |
| chrX | 88978739 | 88978789 |
| chrX | 90283563 | 90283613 |
| chrX | 91489211 | 91489261 |
| chrX | 92067282 | 92067332 |
| chrX | 98270218 | 98270268 |
| chrX | 101446981 | 101447031 |
| chrX | 104114023 | 104114073 |
| chr11 | 9758708 | 9758758 |
| chrX | 109423715 | 109423765 |
| chrX | 109644277 | 109644327 |
| chrX | 115282502 | 115282552 |
| chrX | 115284223 | 115284273 |
| chrX | 118930248 | 118930298 |
| chrX | 119261973 | 119262023 |
| chrX | 125201551 | 125201601 |
| chrX | 128413316 | 128413366 |
| chrX | 129714867 | 129714917 |
| chrX | 138959582 | 138959632 |
| chrX | 139810344 | 139810394 |
| chrX | 143283619 | 143283669 |
| chrX | 147627879 | 147627929 |
| chrX | 147630067 | 147630117 |
| chrX | 148332296 | 148332346 |
| chrX | 150986787 | 150986837 |
| chr11 | 13451280 | 13451330 |
| chrX | 151740030 | 151740080 |
| chrX | 153124337 | 153124387 |
| chrY | 5275229 | 5275279 |
| chrY | 22280806 | 22280856 |
| chr2 | 855052 | 855102 |
| chr2 | 1327377 | 1327427 |
| chr2 | 2051572 | 2051622 |
| chr11 | 16062323 | 16062373 |
| chr11 | 16062979 | 16063029 |
| chr2 | 4953172 | 4953222 |
| chr2 | 5543347 | 5543397 |
| chr2 | 6656045 | 6656095 |
| chr2 | 11561259 | 11561309 |
| chr2 | 11843539 | 11843589 |
| chr2 | 21384804 | 21384854 |
| chr2 | 27228368 | 27228418 |
| chr2 | 37943678 | 37943728 |
| chr2 | 38513946 | 38513996 |
| chr2 | 42302034 | 42302084 |
| chr2 | 46408570 | 46408620 |
| chr2 | 53909678 | 53909728 |
| chr2 | 61853286 | 61853336 |
| chr2 | 61975369 | 61975419 |
| chr11 | 23624442 | 23624492 |
| chr2 | 63955323 | 63955373 |
| chr2 | 65214840 | 65214890 |
| chr2 | 68695855 | 68695905 |
| chr11 | 25286792 | 25286842 |
| chr2 | 70620206 | 70620256 |
| chr2 | 73940191 | 73940241 |
| chr2 | 75927671 | 75927721 |
| chr2 | 79204062 | 79204112 |
| chr2 | 81768009 | 81768059 |
| chr2 | 86761525 | 86761575 |
| chr2 | 86845934 | 86845984 |
| chr2 | 97142279 | 97142329 |
| chr2 | 101375431 | 101375481 |
| chr2 | 104537746 | 104537796 |
| chr2 | 106535639 | 106535689 |
| chr2 | 107255191 | 107255241 |
| chr2 | 115423706 | 115423756 |
| chr2 | 117881546 | 117881596 |
| chr2 | 120543299 | 120543349 |
| chr2 | 129467410 | 129467460 |
| chr2 | 132247539 | 132247589 |
| chr2 | 132435344 | 132435394 |
| chr2 | 132781839 | 132781889 |
| chr2 | 134059821 | 134059871 |
| chr2 | 135045643 | 135045693 |
| chr2 | 143654440 | 143654490 |
| chr2 | 158435665 | 158435715 |
| chr2 | 158561622 | 158561672 |
| chr11 | 35539748 | 35539798 |
| chr2 | 173750757 | 173750807 |
| chr2 | 176107921 | 176107971 |
| chr2 | 180134858 | 180134908 |
| chr2 | 180187590 | 180187640 |
| chr2 | 180376220 | 180376270 |
| chr2 | 183445032 | 183445082 |
| chr11 | 38518519 | 38518569 |
| chr2 | 189623995 | 189624045 |
| chr2 | 190825264 | 190825314 |
| chr2 | 191468339 | 191468389 |
| chr2 | 194041993 | 194042043 |
| chr2 | 201241943 | 201241993 |
| chr2 | 203411046 | 203411096 |
| chr2 | 204326801 | 204326851 |
| chr11 | 40716816 | 40716866 |
| chr2 | 205710604 | 205710654 |
| chr2 | 205897391 | 205897441 |
| chr2 | 209127422 | 209127472 |
| chr2 | 212783315 | 212783365 |
| chr2 | 216245556 | 216245606 |
| chr2 | 221868986 | 221869036 |
| chr2 | 225788560 | 225788610 |
| chr2 | 228752354 | 228752404 |
| chr2 | 229665053 | 229665103 |
| chr2 | 231495633 | 231495683 |
| chr2 | 232039565 | 232039615 |
| chr2 | 237140789 | 237140839 |
| chr2 | 237450368 | 237450418 |
| chr11 | 45826640 | 45826690 |
| chr2 | 240177162 | 240177212 |
| chr2 | 240258521 | 240258571 |
| chr2 | 240687231 | 240687281 |
| chr2 | 240955994 | 240956044 |
| chr2 | 241452682 | 241452732 |
| chr2 | 241790134 | 241790184 |
| chr2 | 242990764 | 242990814 |
| chr2 | 243250171 | 243250221 |
| chr3 | 3138199 | 3138249 |
| chr3 | 8761838 | 8761888 |
| chr3 | 8761533 | 8761583 |
| chr3 | 14988961 | 14989011 |
| chr3 | 23116559 | 23116609 |
| chr3 | 24005272 | 24005322 |
| chr3 | 32714680 | 32714730 |
| chr10 | 12458739 | 12458789 |
| chr3 | 45240122 | 45240172 |
| chr3 | 47815106 | 47815156 |
| chr3 | 50734107 | 50734157 |
| chr3 | 54093250 | 54093300 |
| chr11 | 56991717 | 56991767 |
| chr3 | 75381757 | 75381807 |
| chr3 | 77709533 | 77709583 |
| chr3 | 80629199 | 80629249 |
| chr3 | 86019685 | 86019735 |
| chr3 | 88990148 | 88990198 |
| chr3 | 96783608 | 96783658 |
| chr3 | 100845901 | 100845951 |
| chr3 | 101219199 | 101219249 |
| chr11 | 59243434 | 59243484 |
| chr3 | 103318236 | 103318286 |
| chr3 | 106096590 | 106096640 |
| chr3 | 116223969 | 116224019 |
| chr3 | 118991853 | 118991903 |
| chr3 | 120152768 | 120152818 |
| chr3 | 123478029 | 123478079 |
| chr3 | 138605672 | 138605722 |
| chr3 | 142022022 | 142022072 |
| chr3 | 147084997 | 147085047 |
| chr3 | 154870852 | 154870902 |
| chr3 | 159181223 | 159181273 |
| chr3 | 164527020 | 164527070 |
| chr3 | 173574851 | 173574901 |
| chr3 | 176977474 | 176977524 |
| chr3 | 179039437 | 179039487 |
| chr3 | 184510826 | 184510876 |
| chr10 | 13003536 | 13003586 |
| chr3 | 192143533 | 192143583 |
| chr3 | 193256818 | 193256868 |
| chr3 | 193669201 | 193669251 |
| chr3 | 194807740 | 194807790 |
| chr3 | 195950575 | 195950625 |
| chr3 | 199104385 | 199104435 |
| chr4 | 450692 | 450742 |
| chr4 | 2000184 | 2000234 |
| chr4 | 3727039 | 3727089 |
| chr4 | 5158919 | 5158969 |
| chr4 | 5471508 | 5471558 |
| chr4 | 10596341 | 10596391 |
| chr4 | 13826446 | 13826496 |
| chr4 | 15486246 | 15486296 |
| chr4 | 15640723 | 15640773 |
| chr4 | 19911333 | 19911383 |
| chr4 | 22618991 | 22619041 |
| chr4 | 24422495 | 24422545 |
| chr4 | 28670052 | 28670102 |
| chr4 | 36572828 | 36572878 |
| chr4 | 38169964 | 38170014 |
| chr4 | 43313853 | 43313903 |
| chr4 | 44160312 | 44160362 |
| chr4 | 53374935 | 53374985 |
| chr4 | 53374657 | 53374707 |
| chr4 | 54038863 | 54038913 |
| chr4 | 62007396 | 62007446 |
| chr4 | 77469265 | 77469315 |
| chr4 | 77616808 | 77616858 |
| chr4 | 77836731 | 77836781 |
| chr4 | 78234526 | 78234576 |
| chr10 | 13437896 | 13437946 |
| chr4 | 84411789 | 84411839 |
| chr4 | 84479609 | 84479659 |
| chr4 | 84867263 | 84867313 |
| chr4 | 87928272 | 87928322 |
| chr4 | 90660950 | 90661000 |
| chr4 | 95513132 | 95513182 |
| chr4 | 95776780 | 95776830 |
| chr4 | 108017000 | 108017050 |
| chr4 | 109868345 | 109868395 |
| chr4 | 120062619 | 120062669 |
| chr4 | 128246962 | 128247012 |
| chr11 | 66515968 | 66516018 |
| chr4 | 132448019 | 132448069 |
| chr4 | 134330513 | 134330563 |
| chr4 | 137075973 | 137076023 |
| chr4 | 137321225 | 137321275 |
| chr4 | 138218022 | 138218072 |
| chr4 | 144489594 | 144489644 |
| chr4 | 149641092 | 149641142 |
| chr4 | 154214420 | 154214470 |
| chr4 | 157952073 | 157952123 |
| chr4 | 158549665 | 158549715 |
| chr4 | 159769355 | 159769405 |
| chr4 | 164038975 | 164039025 |
| chr4 | 164727053 | 164727103 |
| chr4 | 166271186 | 166271236 |
| chr4 | 166297456 | 166297506 |
| chr4 | 173240078 | 173240128 |
| chr4 | 182150208 | 182150258 |
| chr4 | 183681750 | 183681800 |
| chr4 | 183682808 | 183682858 |
| chr4 | 185950684 | 185950734 |
| chr4 | 186738753 | 186738803 |
| chr4 | 186739076 | 186739126 |
| chr11 | 68505537 | 68505587 |
| chr5 | 2320972 | 2321022 |
| chr5 | 2575289 | 2575339 |
| chr5 | 4331870 | 4331920 |
| chr5 | 4745567 | 4745617 |
| chr11 | 69110189 | 69110239 |
| chr5 | 7990087 | 7990137 |
| chr5 | 12819201 | 12819251 |
| chr5 | 12919543 | 12919593 |
| chr5 | 17215200 | 17215250 |
| chr5 | 20064688 | 20064738 |
| chr5 | 20954939 | 20954989 |
| chr11 | 69570417 | 69570467 |
| chr5 | 23290344 | 23290394 |
| chr5 | 24214950 | 24215000 |
| chr5 | 24215873 | 24215923 |
| chr5 | 24215682 | 24215732 |
| chr5 | 25767537 | 25767587 |
| chr5 | 25932853 | 25932903 |
| chr5 | 26081418 | 26081468 |
| chr5 | 26322417 | 26322467 |
| chr5 | 34340955 | 34341005 |
| chr5 | 35994756 | 35994806 |
| chr5 | 52917983 | 52918033 |
| chr5 | 55985412 | 55985462 |
| chr5 | 56594539 | 56594589 |
| chr5 | 56887679 | 56887729 |
| chr11 | 70531815 | 70531865 |
| chr5 | 77202934 | 77202984 |
| chr5 | 78547584 | 78547634 |
| chr11 | 70854433 | 70854483 |
| chr11 | 70898692 | 70898742 |
| chr5 | 81327129 | 81327179 |
| chr5 | 86700779 | 86700829 |
| chr5 | 86700589 | 86700639 |
| chr5 | 88708465 | 88708515 |
| chr5 | 94908683 | 94908733 |
| chr5 | 101902448 | 101902498 |
| chr5 | 101903695 | 101903745 |
| chr5 | 103022756 | 103022806 |
| chr5 | 104511195 | 104511245 |
| chr5 | 108647406 | 108647456 |
| chr5 | 109396103 | 109396153 |
| chr5 | 110563659 | 110563709 |
| chr5 | 110998474 | 110998524 |
| chr5 | 114157001 | 114157051 |
| chr5 | 114212915 | 114212965 |
| chr5 | 116234548 | 116234598 |
| chr11 | 71946042 | 71946092 |
| chr5 | 119558610 | 119558660 |
| chr5 | 125568950 | 125569000 |
| chr5 | 125950085 | 125950135 |
| chr5 | 127591699 | 127591749 |
| chr5 | 131595596 | 131595646 |
| chr5 | 139028053 | 139028103 |
| chr5 | 152241936 | 152241986 |
| chr5 | 162752278 | 162752328 |
| chr5 | 171334396 | 171334446 |
| chr5 | 175616164 | 175616214 |
| chr10 | 14867515 | 14867565 |
| chr5 | 177242404 | 177242454 |
| chr5 | 178542374 | 178542424 |
| chr5 | 178979782 | 178979832 |
| chr6 | 1433847 | 1433897 |
| chr6 | 1491119 | 1491169 |
| chr6 | 1490930 | 1490980 |
| chr6 | 1503767 | 1503817 |
| chr6 | 3408935 | 3408985 |
| chr6 | 3509322 | 3509372 |
| chr6 | 3817648 | 3817698 |
| chr6 | 3911509 | 3911559 |
| chr11 | 77288589 | 77288639 |
| chr6 | 10454752 | 10454802 |
| chr6 | 10715648 | 10715698 |
| chr6 | 11552771 | 11552821 |
| chr11 | 77951053 | 77951103 |
| chr11 | 78164420 | 78164470 |
| chr6 | 16197533 | 16197583 |
| chr6 | 19755953 | 19756003 |
| chr6 | 26619380 | 26619430 |
| chr6 | 27073075 | 27073125 |
| chr6 | 28717235 | 28717285 |
| chr6 | 30937665 | 30937715 |
| chr11 | 84207794 | 84207844 |
| chr6 | 34182240 | 34182290 |
| chr6 | 37826106 | 37826156 |
| chr11 | 85546999 | 85547049 |
| chr11 | 86055967 | 86056017 |
| chr6 | 52568954 | 52569004 |
| chr11 | 88641909 | 88641959 |
| chr6 | 62552326 | 62552376 |
| chr6 | 79274651 | 79274701 |
| chr6 | 82556299 | 82556349 |
| chr6 | 95047640 | 95047690 |
| chr6 | 104432659 | 104432709 |
| chr11 | 94421798 | 94421848 |
| chr11 | 94420956 | 94421006 |
| chr6 | 113304952 | 113305002 |
| chr6 | 114278635 | 114278685 |
| chr6 | 117183167 | 117183217 |
| chr6 | 122740995 | 122741045 |
| chr6 | 122740806 | 122740856 |
| chr6 | 124185296 | 124185346 |
| chr6 | 131719698 | 131719748 |
| chr6 | 135779034 | 135779084 |
| chr6 | 139867581 | 139867631 |
| chr6 | 141441368 | 141441418 |
| chr6 | 141440987 | 141441037 |
| chr11 | 98874697 | 98874747 |
| chr6 | 147962352 | 147962402 |
| chr6 | 155340213 | 155340263 |
| chr6 | 155642194 | 155642244 |
| chr6 | 158725212 | 158725262 |
| chr6 | 160157758 | 160157808 |
| chr6 | 160209918 | 160209968 |
| chr6 | 161530351 | 161530401 |
| chr6 | 163446845 | 163446895 |
| chr6 | 163949624 | 163949674 |
| chr6 | 163949816 | 163949866 |
| chr6 | 165883566 | 165883616 |
| chr11 | 103629525 | 103629575 |
| chr6 | 167979835 | 167979885 |
| chr6 | 168947624 | 168947674 |
| chr7 | 1996521 | 1996571 |
| chr7 | 4194131 | 4194181 |
| chr7 | 4278482 | 4278532 |
| chr7 | 4363467 | 4363517 |
| chr7 | 4932902 | 4932952 |
| chr7 | 6495299 | 6495349 |
| chr11 | 109940997 | 109941047 |
| chr7 | 10250728 | 10250778 |
| chr7 | 21510479 | 21510529 |
| chr7 | 21740931 | 21740981 |
| chr7 | 24709939 | 24709989 |
| chr7 | 25136717 | 25136767 |
| chr7 | 25574649 | 25574699 |
| chr10 | 16809104 | 16809154 |
| chr11 | 112401160 | 112401210 |
| chr7 | 47691443 | 47691493 |
| chr7 | 55949102 | 55949152 |
| chr7 | 56286433 | 56286483 |
| chr7 | 62058117 | 62058167 |
| chr7 | 64938783 | 64938833 |
| chr7 | 66455221 | 66455271 |
| chr7 | 66556504 | 66556554 |
| chr7 | 66636502 | 66636552 |
| chr7 | 66820367 | 66820417 |
| chr7 | 67061555 | 67061605 |
| chr7 | 68293984 | 68294034 |
| chr7 | 68411773 | 68411823 |
| chr7 | 69711767 | 69711817 |
| chr7 | 70603526 | 70603576 |
| chr7 | 70745414 | 70745464 |
| chr7 | 71368032 | 71368082 |
| chr7 | 74766342 | 74766392 |
| chr7 | 85589638 | 85589688 |
| chr7 | 86451381 | 86451431 |
| chr7 | 87272616 | 87272666 |
| chr7 | 89579608 | 89579658 |
| chr7 | 89683151 | 89683201 |
| chr7 | 93570334 | 93570384 |
| chr7 | 96838755 | 96838805 |
| chr10 | 17331718 | 17331768 |
| chr10 | 17383144 | 17383194 |
| chr7 | 112559594 | 112559644 |
| chr7 | 116426563 | 116426613 |
| chr7 | 119161639 | 119161689 |
| chr7 | 120844869 | 120844919 |
| chr7 | 123724604 | 123724654 |
| chr7 | 124899660 | 124899710 |
| chr7 | 124928372 | 124928422 |
| chr7 | 127507809 | 127507859 |
| chr7 | 128710159 | 128710209 |
| chr10 | 17466616 | 17466666 |
| chr7 | 129424449 | 129424499 |
| chr7 | 130666503 | 130666553 |
| chr7 | 137493426 | 137493476 |
| chr7 | 143550047 | 143550097 |
| chr7 | 145931468 | 145931518 |
| chr7 | 147706126 | 147706176 |
| chr7 | 151642736 | 151642786 |
| chr7 | 152109282 | 152109332 |
| chr7 | 154777339 | 154777389 |
| chr7 | 155680694 | 155680744 |
| chr7 | 158525233 | 158525283 |
| chr8 | 564441 | 564491 |
| chr8 | 2421727 | 2421777 |
| chr8 | 2697980 | 2698030 |
| chr8 | 4762611 | 4762661 |
| chr8 | 8468663 | 8468713 |
| chr8 | 10324180 | 10324230 |
| chr8 | 10407752 | 10407802 |
| chr8 | 11159932 | 11159982 |
| chr8 | 11160137 | 11160187 |
| chr8 | 11378525 | 11378575 |
| chr8 | 14325851 | 14325901 |
| chr8 | 14423074 | 14423124 |
| chr8 | 18275906 | 18275956 |
| chr8 | 22835811 | 22835861 |
| chr8 | 23068626 | 23068676 |
| chr8 | 25024667 | 25024717 |
| chr8 | 27380149 | 27380199 |
| chr8 | 27380000 | 27380050 |
| chr10 | 18528522 | 18528572 |
| chr8 | 28133796 | 28133846 |
| chr8 | 28133717 | 28133767 |
| chr8 | 30045092 | 30045142 |
| chr8 | 33630490 | 33630540 |
| chr8 | 39862493 | 39862543 |
| chr8 | 42552931 | 42552981 |
| chr8 | 43144505 | 43144555 |
| chr8 | 57525596 | 57525646 |
| chr8 | 61489135 | 61489185 |
| chr8 | 62503295 | 62503345 |
| chr8 | 67684792 | 67684842 |
| chr10 | 18785239 | 18785289 |
| chr8 | 68351326 | 68351376 |
| chr8 | 70883033 | 70883083 |
| chr8 | 77410301 | 77410351 |
| chr8 | 79678603 | 79678653 |
| chr8 | 88198468 | 88198518 |
| chr8 | 90163152 | 90163202 |
| chr8 | 94630400 | 94630450 |
| chr8 | 98360963 | 98361013 |
| chr8 | 98639547 | 98639597 |
| chr8 | 102302911 | 102302961 |
| chr8 | 108985598 | 108985648 |
| chr8 | 110379395 | 110379445 |
| chr8 | 115083089 | 115083139 |
| chr8 | 119483625 | 119483675 |
| chr8 | 129178137 | 129178187 |
| chr8 | 136795721 | 136795771 |
| chr8 | 138469726 | 138469776 |
| chr8 | 139014008 | 139014058 |
| chr8 | 139927234 | 139927284 |
| chr8 | 140228895 | 140228945 |
| chr8 | 140774429 | 140774479 |
| chr8 | 141998125 | 141998175 |
| chr8 | 143845465 | 143845515 |
| chr8 | 145339557 | 145339607 |
| chr9 | 649916 | 649966 |
| chr9 | 2190948 | 2190998 |
| chr9 | 2756222 | 2756272 |
| chr9 | 2853515 | 2853565 |
| chr9 | 11378373 | 11378423 |
| chr9 | 14481895 | 14481945 |
| chr9 | 15377166 | 15377216 |
| chr9 | 19156244 | 19156294 |
| chr9 | 23319152 | 23319202 |
| chr9 | 34156467 | 34156517 |
| chr11 | 133273539 | 133273589 |
| chr9 | 34921059 | 34921109 |
| chr9 | 35193696 | 35193746 |
| chr9 | 70940246 | 70940296 |
| chr9 | 71331324 | 71331374 |
| chr9 | 73317957 | 73318007 |
| chr9 | 74993718 | 74993768 |
| chr9 | 76603008 | 76603058 |
| chr9 | 79564681 | 79564731 |
| chr9 | 79807762 | 79807812 |
| chr9 | 82260135 | 82260185 |
| chr9 | 82271253 | 82271303 |
| chr9 | 85039294 | 85039344 |
| chr9 | 87763093 | 87763143 |
| chr9 | 89412988 | 89413038 |
| chr9 | 90553439 | 90553489 |
| chr9 | 91993325 | 91993375 |
| chr9 | 93533183 | 93533233 |
| chr9 | 102446592 | 102446642 |
| chr9 | 106985243 | 106985293 |
| chr9 | 115060157 | 115060207 |
| chr9 | 115059518 | 115059568 |
| chr9 | 117604438 | 117604488 |
| chr12 | 1373860 | 1373910 |
| chr9 | 123930463 | 123930513 |
| chr9 | 124612046 | 124612096 |
| chr9 | 127713084 | 127713134 |
| chr12 | 2648458 | 2648508 |
| chr12 | 5122384 | 5122434 |
| chr12 | 8829758 | 8829808 |
| chr12 | 8840278 | 8840328 |
| chr12 | 11661097 | 11661147 |
| chr12 | 13446042 | 13446092 |
| chr10 | 24150143 | 24150193 |
| chr12 | 32985218 | 32985268 |
| chr12 | 40701875 | 40701925 |
| chr12 | 47888917 | 47888967 |
| chr12 | 64164429 | 64164479 |
| chr12 | 65487403 | 65487453 |
| chr12 | 67267458 | 67267508 |
| chr12 | 70530676 | 70530726 |
| chr12 | 80076193 | 80076243 |
| chr12 | 82004602 | 82004652 |
| chr12 | 83452288 | 83452338 |
| chr12 | 88425222 | 88425272 |
| chr12 | 89743894 | 89743944 |
| chr12 | 90019051 | 90019101 |
| chr12 | 90865580 | 90865630 |
| chr12 | 91935766 | 91935816 |
| chr10 | 1218632 | 1218682 |
| chr12 | 94952700 | 94952750 |
| chr12 | 102994840 | 102994890 |
| chr12 | 108462314 | 108462364 |
| chr10 | 31353643 | 31353693 |
| chr12 | 116743998 | 116744048 |
| chr12 | 120451082 | 120451132 |
| chr10 | 32902882 | 32902932 |
| chr12 | 122730405 | 122730455 |
| chr12 | 122730225 | 122730275 |
| chr10 | 33560209 | 33560259 |
| chr12 | 124427256 | 124427306 |
| chr12 | 125695426 | 125695476 |
| chr12 | 125878837 | 125878887 |
| chr12 | 126495411 | 126495461 |
| chr12 | 127777331 | 127777381 |
| chr12 | 128849060 | 128849110 |
| chr12 | 128895101 | 128895151 |
| chr12 | 129406973 | 129407023 |
| chr12 | 129888528 | 129888578 |
| chr12 | 130103030 | 130103080 |
| chr12 | 130366071 | 130366121 |
| chr12 | 130699586 | 130699636 |
| chr12 | 131823845 | 131823895 |
| chr12 | 131823914 | 131823964 |
| chr12 | 131824375 | 131824425 |
| chr10 | 35538681 | 35538731 |
| chr10 | 35538922 | 35538972 |
| chr13 | 19402414 | 19402464 |
| chr13 | 20853529 | 20853579 |
| chr13 | 28461102 | 28461152 |
| chr13 | 31733579 | 31733629 |
| chr13 | 32431006 | 32431056 |
| chr13 | 32967730 | 32967780 |
| chr13 | 35010477 | 35010527 |
| chr13 | 37809657 | 37809707 |
| chr13 | 39577745 | 39577795 |
| chr13 | 43000090 | 43000140 |
| chr13 | 43736929 | 43736979 |
| chr13 | 45704636 | 45704686 |
| chr13 | 48030212 | 48030262 |
| chr13 | 49541967 | 49542017 |
| chr13 | 49547155 | 49547205 |
| chr13 | 53707291 | 53707341 |
| chr13 | 54472803 | 54472853 |
| chr13 | 61471614 | 61471664 |
| chr13 | 61530051 | 61530101 |
| chr13 | 73708215 | 73708265 |
| chr13 | 74091482 | 74091532 |
| chr13 | 76224118 | 76224168 |
| chr13 | 77124547 | 77124597 |
| chr13 | 77652117 | 77652167 |
| chr13 | 78795969 | 78796019 |
| chr13 | 80459857 | 80459907 |
| chr13 | 83579699 | 83579749 |
| chr13 | 84830590 | 84830640 |
| chr13 | 88613062 | 88613112 |
| chr13 | 90772051 | 90772101 |
| chr13 | 91381860 | 91381910 |
| chr13 | 93754404 | 93754454 |
| chr13 | 99776196 | 99776246 |
| chr13 | 101063117 | 101063167 |
| chr13 | 102073633 | 102073683 |
| chr13 | 104143792 | 104143842 |
| chr13 | 107979735 | 107979785 |
| chr13 | 109723080 | 109723130 |
| chr13 | 111854214 | 111854264 |
| chr13 | 112673322 | 112673372 |
| chr13 | 112808979 | 112809029 |
| chr14 | 24719699 | 24719749 |
| chr14 | 29317806 | 29317856 |
| chr14 | 29860446 | 29860496 |
| chr14 | 32764595 | 32764645 |
| chr14 | 35515904 | 35515954 |
| chr14 | 36469551 | 36469601 |
| chr10 | 49892592 | 49892642 |
| chr14 | 52293911 | 52293961 |
| chr14 | 61795563 | 61795613 |
| chr14 | 62136518 | 62136568 |
| chr14 | 62260629 | 62260679 |
| chr14 | 63572535 | 63572585 |
| chr14 | 65008028 | 65008078 |
| chr14 | 70535491 | 70535541 |
| chr14 | 70534968 | 70535018 |
| chr14 | 70751724 | 70751774 |
| chr14 | 70783276 | 70783326 |
| chr14 | 73208700 | 73208750 |
| chr14 | 83804588 | 83804638 |
| chr10 | 54112943 | 54112993 |
| chr14 | 85934537 | 85934587 |
| chr14 | 93456762 | 93456812 |
| chr14 | 93700748 | 93700798 |
| chr10 | 56302678 | 56302728 |
| chr14 | 97429849 | 97429899 |
| chr14 | 99628899 | 99628949 |
| chr14 | 100610040 | 100610090 |
| chr10 | 60096274 | 60096324 |
| chr14 | 104814637 | 104814687 |
| chr14 | 105171630 | 105171680 |
| chr15 | 18522283 | 18522333 |
| chr15 | 19534114 | 19534164 |
| chr15 | 21595347 | 21595397 |
| chr15 | 21721523 | 21721573 |
| chr15 | 22126981 | 22127031 |
| chr15 | 24310105 | 24310155 |
| chr15 | 24896180 | 24896230 |
| chr15 | 35413729 | 35413779 |
| chr15 | 36461146 | 36461196 |
| chr15 | 40121049 | 40121099 |
| chr15 | 56225581 | 56225631 |
| chr15 | 56876836 | 56876886 |
| chr15 | 61180372 | 61180422 |
| chr15 | 62250393 | 62250443 |
| chr15 | 75222816 | 75222866 |
| chr15 | 89225910 | 89225960 |
| chr15 | 89736782 | 89736832 |
| chr15 | 89968575 | 89968625 |
| chr15 | 91062044 | 91062094 |
| chr15 | 91387142 | 91387192 |
| chr15 | 91428888 | 91428938 |
| chr16 | 2345203 | 2345253 |
| chr16 | 5423762 | 5423812 |
| chr16 | 5459396 | 5459446 |
| chr16 | 5834873 | 5834923 |
| chr16 | 6016690 | 6016740 |
| chr16 | 7315657 | 7315707 |
| chr16 | 9240767 | 9240817 |
| chr16 | 15623946 | 15623996 |
| chr16 | 15942405 | 15942455 |
| chr16 | 16702587 | 16702637 |
| chr16 | 18694550 | 18694600 |
| chr16 | 48737667 | 48737717 |
| chr16 | 51517247 | 51517297 |
| chr16 | 52451181 | 52451231 |
| chr16 | 64580474 | 64580524 |
| chr16 | 73460518 | 73460568 |
| chr16 | 77189268 | 77189318 |
| chr16 | 86805809 | 86805859 |
| chr17 | 5947356 | 5947406 |
| chr17 | 6827909 | 6827959 |
| chr17 | 7186914 | 7186964 |
| chr17 | 13161443 | 13161493 |
| chr17 | 15619490 | 15619540 |
| chr17 | 20677860 | 20677910 |
| chr17 | 30905573 | 30905623 |
| chr17 | 33281662 | 33281712 |
| chr17 | 35270809 | 35270859 |

Table 15DP. Repeat Category MSTB

| | | |
|---|---|---|
| chr10 | 82267400 | 82267450 |
| chr17 | 48567557 | 48567607 |
| chr17 | 54421646 | 54421696 |
| chr17 | 55351216 | 55351266 |
| chr17 | 56159826 | 56159876 |
| chr17 | 57505478 | 57505528 |
| chr10 | 86464464 | 86464514 |
| chr17 | 66575703 | 66575753 |
| chr17 | 67741387 | 67741437 |
| chr17 | 70585186 | 70585236 |
| chr17 | 75729938 | 75729988 |
| chr17 | 76052877 | 76052927 |
| chr17 | 78945383 | 78945433 |
| chr17 | 80912139 | 80912189 |
| chr17 | 80913287 | 80913337 |
| chr17 | 81608104 | 81608154 |
| chr18 | 993795 | 993845 |
| chr18 | 2701766 | 2701816 |
| chr10 | 95386206 | 95386256 |
| chr18 | 27389520 | 27389570 |
| chr18 | 29626733 | 29626783 |
| chr18 | 46096167 | 46096217 |
| chr18 | 49314504 | 49314554 |
| chr10 | 5241164 | 5241214 |
| chr18 | 58320407 | 58320457 |
| chr10 | 5291100 | 5291150 |
| chr18 | 61887606 | 61887656 |
| chr18 | 64211715 | 64211765 |
| chr18 | 67370922 | 67370972 |
| chr18 | 69950420 | 69950470 |
| chr18 | 72270481 | 72270531 |
| chr18 | 72492632 | 72492682 |
| chr18 | 74944223 | 74944273 |
| chr18 | 74944003 | 74944053 |
| chr19 | 339779 | 339829 |
| chr19 | 605054 | 605104 |
| chr19 | 1656176 | 1656226 |
| chr19 | 5719746 | 5719796 |
| chr19 | 6087882 | 6087932 |
| chr19 | 10987160 | 10987210 |
| chr19 | 11090057 | 11090107 |
| chr19 | 11607484 | 11607534 |
| chr19 | 14394306 | 14394356 |
| chr19 | 14957053 | 14957103 |
| chr19 | 17473222 | 17473272 |
| chr19 | 20376437 | 20376487 |
| chr19 | 33636370 | 33636420 |
| chr19 | 34631099 | 34631149 |
| chr19 | 34630959 | 34631009 |
| chr19 | 34721205 | 34721255 |
| chr19 | 34753498 | 34753548 |
| chr19 | 35538073 | 35538123 |
| chr19 | 39048190 | 39048240 |
| chr19 | 39249812 | 39249862 |
| chr19 | 40166551 | 40166601 |
| chr19 | 40418709 | 40418759 |
| chr10 | 105192813 | 105192863 |
| chr10 | 105193243 | 105193293 |
| chr10 | 105193529 | 105193579 |
| chr10 | 109471526 | 109471576 |
| chr10 | 110237634 | 110237684 |
| chr10 | 111367999 | 111368049 |
| chr19 | 53296313 | 53296363 |
| chr19 | 54387119 | 54387169 |
| chr19 | 55937974 | 55938024 |
| chr19 | 56327657 | 56327707 |
| chr19 | 63277426 | 63277476 |
| chr19 | 63404110 | 63404160 |
| chr1 | 5261741 | 5261791 |
| chr1 | 5268962 | 5269012 |
| chr1 | 7766425 | 7766475 |
| chr1 | 10224409 | 10224459 |
| chr1 | 11686179 | 11686229 |
| chr1 | 13647332 | 13647382 |
| chr1 | 14216346 | 14216396 |
| chr1 | 14276778 | 14276828 |
| chr1 | 23391555 | 23391605 |
| chr1 | 26126487 | 26126537 |
| chr1 | 27638503 | 27638553 |
| chr1 | 38623270 | 38623320 |
| chr10 | 121306349 | 121306399 |
| chr1 | 40368769 | 40368819 |
| chr1 | 43120082 | 43120132 |
| chr1 | 46207384 | 46207434 |
| chr1 | 46839776 | 46839826 |
| chr1 | 47083044 | 47083094 |
| chr1 | 51412978 | 51413028 |
| chr1 | 57886437 | 57886487 |
| chr1 | 58028929 | 58028979 |
| chr1 | 58989326 | 58989376 |
| chr1 | 78623180 | 78623230 |
| chr1 | 90255661 | 90255711 |
| chr10 | 125379054 | 125379104 |
| chr10 | 125378842 | 125378892 |
| chr1 | 98585743 | 98585793 |
| chr1 | 105259936 | 105259986 |
| chr1 | 108556221 | 108556271 |
| chr10 | 6980480 | 6980530 |
| chr1 | 119147565 | 119147615 |
| chr1 | 141840777 | 141840827 |
| chr10 | 126851506 | 126851556 |
| chr1 | 155238863 | 155238913 |
| chr1 | 156186582 | 156186632 |
| chr1 | 156186356 | 156186406 |
| chr10 | 128567589 | 128567639 |
| chr1 | 164029617 | 164029667 |
| chr10 | 128713181 | 128713231 |
| chr1 | 173130455 | 173130505 |
| chr1 | 181966118 | 181966168 |
| chr1 | 181966035 | 181966085 |
| chr1 | 182616085 | 182616135 |
| chr1 | 186117807 | 186117857 |
| chr1 | 191578364 | 191578414 |
| chr1 | 198937535 | 198937585 |
| chr1 | 199618553 | 199618603 |
| chr1 | 203798896 | 203798946 |
| chr1 | 210127515 | 210127565 |
| chr1 | 210126499 | 210126549 |
| chr1 | 222762174 | 222762224 |
| chr1 | 225213113 | 225213163 |
| chr1 | 225589050 | 225589100 |
| chr1 | 227455774 | 227455824 |
| chr1 | 227477120 | 227477170 |
| chr1 | 228770137 | 228770187 |
| chr1 | 229273225 | 229273275 |
| chr1 | 231470261 | 231470311 |
| chr1 | 232564147 | 232564197 |
| chr1 | 240132156 | 240132206 |
| chr1 | 241782545 | 241782595 |
| chr1 | 242675596 | 242675646 |
| chr20 | 669783 | 669833 |
| chr20 | 3248753 | 3248803 |
| chr20 | 5164861 | 5164911 |
| chr20 | 5164726 | 5164776 |
| chr20 | 9103845 | 9103895 |
| chr20 | 13668055 | 13668105 |
| chr20 | 21870781 | 21870831 |
| chr20 | 21978702 | 21978752 |
| chr20 | 31221057 | 31221107 |
| chr20 | 31502272 | 31502322 |
| chr20 | 37725300 | 37725350 |
| chr20 | 37725964 | 37726014 |
| chr20 | 38049078 | 38049128 |
| chr20 | 43892597 | 43892647 |
| chr20 | 46760257 | 46760307 |
| chr20 | 50497274 | 50497324 |
| chr20 | 54930398 | 54930448 |
| chr20 | 55089450 | 55089500 |
| chr20 | 59860107 | 59860157 |
| chr20 | 59860021 | 59860071 |
| chr20 | 60226148 | 60226198 |
| chr20 | 61107948 | 61107998 |
| chr20 | 61363223 | 61363273 |
| chr20 | 61513008 | 61513058 |
| chr10 | 134609524 | 134609574 |
| chr20 | 62858940 | 62858990 |
| chr20 | 63479487 | 63479537 |
| chr21 | 16014761 | 16014811 |
| chr21 | 17042328 | 17042378 |
| chr21 | 17767707 | 17767757 |
| chr21 | 19315579 | 19315629 |
| chr21 | 20978345 | 20978395 |
| chr21 | 24879692 | 24879742 |
| chr21 | 31685436 | 31685486 |
| chr21 | 32601489 | 32601539 |
| chr21 | 33072578 | 33072628 |
| chr21 | 33956644 | 33956694 |
| chr21 | 34859767 | 34859817 |
| chr21 | 35742576 | 35742626 |
| chr21 | 40849941 | 40849991 |
| chr21 | 41524978 | 41525028 |
| chr21 | 42822247 | 42822297 |
| chr21 | 43983344 | 43983394 |
| chr21 | 45821826 | 45821876 |
| chr21 | 45960424 | 45960474 |
| chr21 | 46132711 | 46132761 |
| chr22 | 16104214 | 16104264 |
| chr22 | 16526760 | 16526810 |
| chr22 | 17352086 | 17352136 |
| chr22 | 18408047 | 18408097 |
| chr22 | 20433256 | 20433306 |
| chr22 | 21234838 | 21234888 |
| chr10 | 9167306 | 9167356 |
| chr22 | 22389230 | 22389280 |
| chr11 | 1586938 | 1586988 |
| chr22 | 31477700 | 31477750 |
| chr22 | 33968898 | 33968948 |
| chr10 | 9806118 | 9806168 |
| chr22 | 37851018 | 37851068 |
| chr22 | 41846210 | 41846260 |
| chr22 | 43333786 | 43333836 |
| chr22 | 44991005 | 44991055 |
| chr22 | 46201419 | 46201469 |
| chr22 | 48952030 | 48952080 |
| chrX | 6228634 | 6228684 |
| chrX | 15071533 | 15071583 |
| chrX | 15446093 | 15446143 |
| chrX | 16180541 | 16180591 |
| chr11 | 4786972 | 4787022 |
| chrX | 17782088 | 17782138 |
| chrX | 22043748 | 22043798 |
| chrX | 22612177 | 22612227 |
| chrX | 23247450 | 23247500 |
| chrX | 23832987 | 23833037 |
| chrX | 34433193 | 34433243 |
| chrX | 38739694 | 38739744 |
| chrX | 41806189 | 41806239 |
| chrX | 44839188 | 44839238 |
| chrX | 48016926 | 48016976 |
| chrX | 50608314 | 50608364 |
| chr11 | 7947910 | 7947960 |
| chrX | 52716567 | 52716617 |
| chrX | 53083196 | 53083246 |
| chrX | 53858167 | 53858217 |
| chrX | 55907603 | 55907653 |
| chrX | 70244447 | 70244497 |
| chrX | 71612683 | 71612733 |
| chrX | 71991359 | 71991409 |
| chrX | 72254796 | 72254846 |
| chrX | 81863466 | 81863516 |
| chrX | 85626898 | 85626948 |
| chrX | 86766044 | 86766094 |
| chrX | 87993717 | 87993767 |
| chrX | 88852622 | 88852672 |
| chrX | 93600896 | 93600946 |
| chr11 | 9357816 | 9357866 |
| chrX | 96919060 | 96919110 |
| chrX | 103115935 | 103115985 |
| chrX | 105379088 | 105379138 |
| chrX | 112493158 | 112493208 |
| chrX | 117231013 | 117231063 |
| chrX | 117608871 | 117608921 |
| chrX | 117654850 | 117654900 |
| chrX | 120043842 | 120043892 |
| chrX | 122021593 | 122021643 |
| chrX | 130147829 | 130147879 |
| chrX | 131646562 | 131646612 |
| chrX | 140212967 | 140213017 |
| chr11 | 12490331 | 12490381 |
| chr11 | 12490184 | 12490234 |
| chrX | 147342261 | 147342311 |
| chrY | 6122806 | 6122856 |
| chrY | 7238496 | 7238546 |
| chr2 | 1026934 | 1026984 |
| chr2 | 1765947 | 1765997 |
| chr11 | 15672337 | 15672387 |
| chr2 | 2560762 | 2560812 |
| chr2 | 3178777 | 3178827 |
| chr2 | 12153123 | 12153173 |
| chr2 | 19077283 | 19077333 |
| chr2 | 23378566 | 23378616 |
| chr11 | 18423863 | 18423913 |
| chr2 | 30051458 | 30051508 |
| chr2 | 30408202 | 30408252 |
| chr2 | 43183865 | 43183915 |
| chr2 | 43218810 | 43218860 |
| chr2 | 44953257 | 44953307 |
| chr2 | 46146609 | 46146659 |
| chr2 | 56334979 | 56335029 |
| chr2 | 61543248 | 61543298 |
| chr2 | 61969776 | 61969826 |
| chr11 | 25574739 | 25574789 |
| chr11 | 25574558 | 25574608 |
| chr2 | 85903251 | 85903301 |
| chr2 | 88310080 | 88310130 |
| chr2 | 98044846 | 98044896 |
| chr2 | 99142709 | 99142759 |
| chr2 | 99958083 | 99958133 |
| chr2 | 100031065 | 100031115 |
| chr2 | 104721484 | 104721534 |
| chr2 | 105500461 | 105500511 |
| chr2 | 113642954 | 113643004 |
| chr2 | 115439544 | 115439594 |
| chr2 | 116278574 | 116278624 |
| chr11 | 32532462 | 32532512 |
| chr2 | 120446181 | 120446231 |
| chr2 | 121437900 | 121437950 |
| chr2 | 121457261 | 121457311 |
| chr2 | 129438915 | 129438965 |
| chr2 | 130479931 | 130479981 |
| chr2 | 138594488 | 138594538 |
| chr2 | 143495291 | 143495341 |
| chr2 | 151718328 | 151718378 |
| chr2 | 160619956 | 160620006 |
| chr2 | 166078750 | 166078800 |
| chr2 | 172753827 | 172753877 |
| chr2 | 190743157 | 190743207 |
| chr2 | 194582206 | 194582256 |
| chr2 | 201873073 | 201873123 |
| chr2 | 202178389 | 202178439 |
| chr2 | 202178325 | 202178375 |
| chr2 | 203129931 | 203129981 |
| chr2 | 216982642 | 216982692 |
| chr2 | 220271259 | 220271309 |
| chr2 | 221467729 | 221467779 |
| chr2 | 222615443 | 222615493 |
| chr2 | 231957341 | 231957391 |
| chr2 | 233852119 | 233852169 |
| chr2 | 234980103 | 234980153 |
| chr2 | 235355260 | 235355310 |
| chr2 | 239349756 | 239349806 |
| chr11 | 45801643 | 45801693 |
| chr2 | 240958737 | 240958787 |
| chr2 | 240958652 | 240958702 |
| chr2 | 241454760 | 241454810 |
| chr2 | 241844409 | 241844459 |
| chr2 | 242813698 | 242813748 |
| chr3 | 9192880 | 9192930 |
| chr10 | 12364228 | 12364278 |
| chr3 | 16491880 | 16491930 |
| chr3 | 16491653 | 16491703 |
| chr3 | 17923921 | 17923971 |
| chr3 | 18952998 | 18953048 |
| chr3 | 21631326 | 21631376 |
| chr3 | 28913683 | 28913733 |
| chr3 | 41847584 | 41847634 |
| chr3 | 45140512 | 45140562 |
| chr3 | 49814426 | 49814476 |
| chr3 | 50975974 | 50976024 |
| chr3 | 57884381 | 57884431 |
| chr3 | 60219380 | 60219430 |
| chr3 | 63988270 | 63988320 |
| chr3 | 67052185 | 67052235 |
| chr3 | 80430511 | 80430561 |
| chr3 | 81411685 | 81411735 |
| chr3 | 88176564 | 88176614 |
| chr3 | 99907830 | 99907880 |
| chr3 | 115147980 | 115148030 |
| chr3 | 121040965 | 121041015 |
| chr3 | 129627533 | 129627583 |
| chr3 | 144250831 | 144250881 |
| chr3 | 147400213 | 147400263 |
| chr3 | 150741973 | 150742023 |
| chr3 | 154264772 | 154264822 |
| chr3 | 158176367 | 158176417 |
| chr3 | 168015550 | 168015600 |
| chr3 | 176081746 | 176081796 |
| chr3 | 180812955 | 180813005 |
| chr3 | 182983482 | 182983532 |
| chr3 | 186256268 | 186256318 |
| chr3 | 186287720 | 186287770 |
| chr3 | 186422313 | 186422363 |
| chr3 | 187851359 | 187851409 |
| chr3 | 189553527 | 189553577 |
| chr3 | 192445644 | 192445694 |
| chr3 | 196286041 | 196286091 |
| chr4 | 508270 | 508320 |
| chr4 | 5026171 | 5026221 |
| chr4 | 11753452 | 11753502 |
| chr4 | 14996354 | 14996404 |
| chr4 | 15867733 | 15867783 |
| chr4 | 17136944 | 17136994 |
| chr4 | 18575341 | 18575391 |
| chr4 | 24540849 | 24540899 |
| chr4 | 31192895 | 31192945 |
| chr4 | 46750403 | 46750453 |
| chr4 | 47575168 | 47575218 |
| chr4 | 54937449 | 54937499 |
| chr4 | 67868869 | 67868919 |
| chr4 | 70977706 | 70977756 |
| chr4 | 72208133 | 72208183 |
| chr4 | 90725576 | 90725626 |
| chr4 | 104009332 | 104009382 |
| chr4 | 106744571 | 106744621 |
| chr4 | 117383824 | 117383874 |
| chr4 | 119965655 | 119965705 |
| chr4 | 124762191 | 124762241 |
| chr4 | 125671442 | 125671492 |
| chr4 | 126541857 | 126541907 |
| chr4 | 132366697 | 132366747 |
| chr4 | 134017331 | 134017381 |
| chr4 | 135054156 | 135054206 |
| chr4 | 139626989 | 139627039 |
| chr4 | 140166425 | 140166475 |
| chr4 | 163640181 | 163640231 |
| chr4 | 165130903 | 165130953 |
| chr4 | 168343862 | 168343912 |
| chr4 | 169337258 | 169337308 |
| chr4 | 169337659 | 169337709 |
| chr4 | 177354751 | 177354801 |
| chr4 | 179018082 | 179018132 |
| chr4 | 179237751 | 179237801 |
| chr4 | 180437491 | 180437541 |
| chr4 | 181203613 | 181203663 |
| chr4 | 185075468 | 185075518 |
| chr4 | 185935433 | 185935483 |
| chr4 | 190931367 | 190931417 |
| chr5 | 767762 | 767812 |
| chr5 | 852060 | 852110 |
| chr5 | 2341977 | 2342027 |
| chr5 | 3297205 | 3297255 |
| chr5 | 9184142 | 9184192 |
| chr5 | 15010471 | 15010521 |
| chr5 | 16962395 | 16962445 |
| chr5 | 20852747 | 20852797 |
| chr5 | 26979439 | 26979489 |
| chr5 | 31975672 | 31975722 |
| chr5 | 32230998 | 32231048 |
| chr5 | 34442340 | 34442390 |
| chr5 | 37286089 | 37286139 |
| chr11 | 70015849 | 70015899 |
| chr5 | 43321614 | 43321664 |
| chr5 | 43620931 | 43620981 |
| chr5 | 43620708 | 43620758 |
| chr5 | 55084043 | 55084093 |
| chr5 | 55144305 | 55144355 |
| chr5 | 62116687 | 62116737 |
| chr5 | 62116753 | 62116803 |
| chr5 | 67129551 | 67129601 |
| chr11 | 70553759 | 70553809 |
| chr11 | 70553692 | 70553742 |
| chr5 | 74305828 | 74305878 |
| chr5 | 76127961 | 76128011 |
| chr11 | 70775900 | 70775950 |
| chr5 | 92010868 | 92010918 |
| chr5 | 92195265 | 92195315 |
| chr5 | 93644209 | 93644259 |
| chr5 | 98686461 | 98686511 |
| chr11 | 71946042 | 71946092 |
| chr5 | 118876968 | 118877018 |
| chr5 | 124274859 | 124274909 |
| chr5 | 124276306 | 124276356 |
| chr5 | 133429849 | 133429899 |
| chr11 | 73111150 | 73111200 |
| chr5 | 139672162 | 139672212 |
| chr5 | 154069475 | 154069525 |
| chr5 | 171284437 | 171284487 |
| chr5 | 171284271 | 171284321 |
| chr5 | 172429660 | 172429710 |
| chr5 | 175848410 | 175848460 |
| chr11 | 75898361 | 75898411 |
| chr5 | 177509254 | 177509304 |
| chr5 | 177734065 | 177734115 |
| chr5 | 178484311 | 178484361 |
| chr6 | 3511935 | 3511985 |
| chr6 | 6742591 | 6742641 |
| chr6 | 11484584 | 11484634 |
| chr6 | 14695949 | 14695999 |
| chr6 | 16049543 | 16049593 |
| chr6 | 17279170 | 17279220 |
| chr6 | 17408463 | 17408513 |
| chr6 | 17466319 | 17466369 |
| chr6 | 18134481 | 18134531 |
| chr6 | 22642952 | 22643002 |
| chr6 | 23679967 | 23680017 |
| chr6 | 27446473 | 27446523 |
| chr6 | 32796124 | 32796174 |
| chr10 | 15241373 | 15241423 |
| chr6 | 45408463 | 45408513 |
| chr6 | 47630144 | 47630194 |
| chr6 | 49510392 | 49510442 |
| chr11 | 88237218 | 88237268 |
| chr6 | 63965557 | 63965607 |
| chr11 | 89684981 | 89685031 |
| chr6 | 74398785 | 74398835 |
| chr6 | 76474844 | 76474894 |
| chr6 | 85758144 | 85758194 |
| chr6 | 86022358 | 86022408 |
| chr11 | 92325486 | 92325536 |
| chr6 | 90333708 | 90333758 |
| chr6 | 103686484 | 103686534 |
| chr6 | 107629014 | 107629064 |
| chr6 | 109186186 | 109186236 |
| chr6 | 115624784 | 115624834 |
| chr6 | 116960864 | 116960914 |
| chr6 | 120361677 | 120361727 |
| chr6 | 124817572 | 124817622 |
| chr6 | 124817407 | 124817457 |
| chr6 | 126457377 | 126457427 |
| chr6 | 129824969 | 129825019 |
| chr6 | 131445396 | 131445446 |
| chr6 | 134431790 | 134431840 |
| chr6 | 135074792 | 135074842 |
| chr6 | 135114416 | 135114466 |
| chr6 | 138301200 | 138301250 |
| chr6 | 140255093 | 140255143 |
| chr6 | 143743612 | 143743662 |
| chr6 | 144578458 | 144578508 |
| chr6 | 146916571 | 146916621 |
| chr11 | 100825792 | 100825842 |
| chr6 | 158640582 | 158640632 |
| chr6 | 160156726 | 160156776 |
| chr11 | 101798229 | 101798279 |
| chr11 | 101812586 | 101812636 |
| chr6 | 161345981 | 161346031 |
| chr6 | 166012383 | 166012433 |
| chr7 | 1648753 | 1648803 |
| chr7 | 1916827 | 1916877 |
| chr7 | 3687404 | 3687454 |
| chr11 | 109777172 | 109777222 |
| chr7 | 10859779 | 10859829 |
| chr7 | 15545104 | 15545154 |
| chr7 | 21742240 | 21742290 |
| chr7 | 24507279 | 24507329 |
| chr7 | 26203804 | 26203854 |
| chr11 | 111438717 | 111438767 |
| chr7 | 30166593 | 30166643 |
| chr7 | 30166673 | 30166723 |
| chr11 | 111635965 | 111636015 |
| chr7 | 37470800 | 37470850 |
| chr7 | 44451749 | 44451799 |
| chr7 | 45193199 | 45193249 |
| chr7 | 47457236 | 47457286 |
| chr7 | 63572288 | 63572338 |
| chr7 | 65275522 | 65275572 |
| chr7 | 68411773 | 68411823 |
| chr7 | 70119658 | 70119708 |
| chr7 | 70432871 | 70432921 |
| chr7 | 70659204 | 70659254 |
| chr7 | 70658923 | 70658973 |
| chr7 | 71026673 | 71026723 |
| chr7 | 71276649 | 71276699 |
| chr7 | 72608830 | 72608880 |
| chr7 | 74957472 | 74957522 |
| chr7 | 78741810 | 78741860 |
| chr7 | 89633972 | 89634022 |
| chr7 | 97475261 | 97475311 |
| chr7 | 97570944 | 97570994 |
| chr7 | 98034044 | 98034094 |
| chr7 | 98576035 | 98576085 |
| chr7 | 100267427 | 100267477 |
| chr7 | 100648877 | 100648927 |
| chr7 | 101473407 | 101473457 |
| chr7 | 104234760 | 104234810 |
| chr7 | 113328809 | 113328859 |
| chr7 | 123204395 | 123204445 |
| chr7 | 123930634 | 123930684 |
| chr11 | 118116141 | 118116191 |
| chr7 | 130222718 | 130222768 |
| chr7 | 135138367 | 135138417 |
| chr7 | 143039189 | 143039239 |
| chr11 | 118849783 | 118849833 |
| chr7 | 148058302 | 148058352 |
| chr7 | 150787128 | 150787178 |
| chr7 | 150986282 | 150986332 |
| chr7 | 151059258 | 151059308 |
| chr7 | 151714738 | 151714788 |
| chr7 | 152190369 | 152190419 |
| chr7 | 152238506 | 152238556 |
| chr7 | 154390167 | 154390217 |
| chr7 | 154967890 | 154967940 |
| chr7 | 155463705 | 155463755 |
| chr7 | 156038532 | 156038582 |
| chr7 | 157800037 | 157800087 |
| chr7 | 157799859 | 157799909 |
| chr8 | 1672947 | 1672997 |
| chr8 | 2039844 | 2039894 |
| chr11 | 121402439 | 121402489 |
| chr8 | 4699197 | 4699247 |
| chr8 | 6147929 | 6147979 |
| chr11 | 121705789 | 121705839 |
| chr10 | 18281274 | 18281324 |
| chr8 | 9984662 | 9984712 |
| chr8 | 13686403 | 13686453 |
| chr8 | 16570215 | 16570265 |
| chr11 | 122549858 | 122549908 |
| chr8 | 21283134 | 21283184 |
| chr11 | 122849124 | 122849174 |
| chr11 | 123170295 | 123170345 |
| chr10 | 18536301 | 18536351 |
| chr8 | 32734976 | 32735026 |
| chr8 | 33933912 | 33933962 |
| chr8 | 36267203 | 36267253 |
| chr8 | 37825367 | 37825417 |
| chr8 | 39346162 | 39346212 |
| chr8 | 39617457 | 39617507 |
| chr8 | 40224296 | 40224346 |
| chr8 | 49718688 | 49718738 |
| chr8 | 50121037 | 50121087 |
| chr8 | 55790827 | 55790877 |
| chr8 | 57023351 | 57023401 |
| chr8 | 64126579 | 64126629 |
| chr8 | 72728256 | 72728306 |
| chr8 | 76541573 | 76541623 |
| chr8 | 77022855 | 77022905 |
| chr8 | 81063922 | 81063972 |
| chr8 | 81607390 | 81607440 |
| chr8 | 83576394 | 83576444 |
| chr8 | 94528535 | 94528585 |
| chr8 | 96416556 | 96416606 |
| chr8 | 98401755 | 98401805 |
| chr8 | 98717863 | 98717913 |
| chr8 | 101044009 | 101044059 |
| chr8 | 103428828 | 103428878 |
| chr8 | 112772098 | 112772148 |
| chr8 | 118020662 | 118020712 |
| chr8 | 123648457 | 123648507 |
| chr8 | 126970044 | 126970094 |
| chr8 | 130318850 | 130318900 |
| chr8 | 131500714 | 131500764 |
| chr8 | 134859343 | 134859393 |
| chr8 | 137294881 | 137294931 |
| chr8 | 141924114 | 141924164 |
| chr8 | 144252762 | 144252812 |
| chr8 | 144252687 | 144252737 |
| chr8 | 144441241 | 144441291 |
| chr8 | 144441942 | 144441992 |
| chr8 | 144951500 | 144951550 |
| chr11 | 130929079 | 130929129 |
| chr11 | 131062555 | 131062605 |
| chr8 | 145861459 | 145861509 |
| chr10 | 19806204 | 19806254 |
| chr9 | 6580065 | 6580115 |
| chr11 | 132408352 | 132408402 |
| chr9 | 20713474 | 20713524 |
| chr9 | 36402501 | 36402551 |
| chr9 | 38085827 | 38085877 |
| chr9 | 67341341 | 67341391 |
| chr9 | 81492799 | 81492849 |
| chr9 | 82987343 | 82987393 |
| chr9 | 83622817 | 83622867 |
| chr9 | 83622669 | 83622719 |
| chr9 | 83945559 | 83945609 |
| chr9 | 83946163 | 83946213 |
| chr9 | 83946297 | 83946347 |
| chr9 | 86094921 | 86094971 |
| chr9 | 86095174 | 86095224 |
| chr9 | 86220786 | 86220836 |
| chr9 | 87287030 | 87287080 |
| chr11 | 134025879 | 134025929 |
| chr9 | 103627572 | 103627622 |
| chr9 | 110486938 | 110486988 |
| chr9 | 110881909 | 110881959 |
| chr9 | 116834074 | 116834124 |
| chr9 | 120975059 | 120975109 |
| chr9 | 122052502 | 122052552 |
| chr9 | 128220736 | 128220786 |
| chr9 | 128660480 | 128660530 |
| chr9 | 130046797 | 130046847 |
| chr9 | 130046656 | 130046706 |
| chr10 | 21641642 | 21641692 |
| chr9 | 133167436 | 133167486 |
| chr12 | 4154188 | 4154238 |
| chr12 | 4207661 | 4207711 |
| chr12 | 5690728 | 5690778 |
| chr12 | 7787855 | 7787905 |
| chr12 | 10883590 | 10883640 |
| chr10 | 22870670 | 22870720 |
| chr12 | 21238279 | 21238329 |
| chr10 | 24039681 | 24039731 |
| chr10 | 24057720 | 24057770 |
| chr12 | 26387101 | 26387151 |
| chr12 | 26618966 | 26619016 |
| chr12 | 27357528 | 27357578 |
| chr12 | 33236546 | 33236596 |
| chr12 | 34254285 | 34254335 |
| chr12 | 54563810 | 54563860 |
| chr12 | 61303385 | 61303435 |
| chr12 | 67979557 | 67979607 |
| chr10 | 28926586 | 28926636 |
| chr12 | 76921358 | 76921408 |
| chr12 | 78325177 | 78325227 |
| chr12 | 83876692 | 83876742 |
| chr12 | 84127839 | 84127889 |
| chr12 | 98359524 | 98359574 |
| chr12 | 102853073 | 102853123 |
| chr12 | 112386976 | 112387026 |
| chr12 | 115191144 | 115191194 |
| chr10 | 31447112 | 31447162 |
| chr12 | 116927846 | 116927896 |
| chr12 | 124427256 | 124427306 |
| chr12 | 124640271 | 124640321 |
| chr12 | 126755663 | 126755713 |
| chr12 | 128351001 | 128351051 |
| chr12 | 129609979 | 129610029 |
| chr13 | 18249389 | 18249439 |
| chr13 | 27048486 | 27048536 |
| chr13 | 28646991 | 28647041 |
| chr13 | 29447391 | 29447441 |
| chr13 | 31917515 | 31917565 |
| chr13 | 35106082 | 35106132 |
| chr13 | 39076798 | 39076848 |
| chr13 | 44604251 | 44604301 |
| chr13 | 46799072 | 46799122 |
| chr13 | 46798806 | 46798856 |
| chr13 | 50262066 | 50262116 |
| chr13 | 53462193 | 53462243 |
| chr13 | 53840773 | 53840823 |
| chr13 | 55501386 | 55501436 |
| chr13 | 59800054 | 59800104 |
| chr13 | 65370217 | 65370267 |
| chr13 | 72012773 | 72012823 |
| chr13 | 74499453 | 74499503 |
| chr13 | 76022076 | 76022126 |
| chr13 | 77739914 | 77739964 |
| chr13 | 83837723 | 83837773 |
| chr13 | 84150559 | 84150609 |
| chr13 | 87785426 | 87785476 |
| chr13 | 96701986 | 96702036 |
| chr13 | 97027878 | 97027928 |
| chr13 | 97754562 | 97754612 |
| chr10 | 43522029 | 43522079 |
| chr13 | 106257692 | 106257742 |
| chr13 | 106257604 | 106257654 |
| chr13 | 111649032 | 111649082 |
| chr14 | 22393930 | 22393980 |
| chr14 | 22393844 | 22393894 |
| chr14 | 22658231 | 22658281 |
| chr14 | 32552191 | 32552241 |
| chr14 | 39184578 | 39184628 |
| chr14 | 46510486 | 46510536 |
| chr14 | 49632226 | 49632276 |
| chr14 | 51652487 | 51652537 |
| chr10 | 50861784 | 50861834 |
| chr14 | 66800897 | 66800947 |
| chr14 | 71878938 | 71878988 |
| chr14 | 73514365 | 73514415 |
| chr14 | 73515627 | 73515677 |
| chr10 | 52923500 | 52923550 |
| chr14 | 75527757 | 75527807 |
| chr14 | 79773603 | 79773653 |
| chr14 | 79785522 | 79785572 |
| chr14 | 80264639 | 80264689 |
| chr14 | 83809998 | 83810048 |
| chr14 | 84851761 | 84851811 |
| chr14 | 90218239 | 90218289 |
| chr14 | 93860396 | 93860446 |
| chr14 | 94249122 | 94249172 |
| chr14 | 98418533 | 98418583 |
| chr14 | 100842001 | 100842051 |
| chr14 | 104880917 | 104880967 |
| chr15 | 21727893 | 21727943 |
| chr15 | 27487486 | 27487536 |
| chr15 | 38538232 | 38538282 |
| chr15 | 53356617 | 53356667 |
| chr10 | 65518292 | 65518342 |
| chr15 | 57763922 | 57763972 |
| chr15 | 65933522 | 65933572 |
| chr15 | 72919310 | 72919360 |
| chr15 | 72921328 | 72921378 |
| chr15 | 74756056 | 74756106 |
| chr15 | 84997327 | 84997377 |
| chr15 | 90837864 | 90837914 |
| chr15 | 91869794 | 91869844 |
| chr15 | 96698975 | 96699025 |
| chr16 | 38494 | 38544 |
| chr16 | 1281875 | 1281925 |
| chr16 | 1803005 | 1803055 |
| chr16 | 11443726 | 11443776 |
| chr16 | 12221655 | 12221705 |
| chr16 | 12222473 | 12222523 |
| chr16 | 12476621 | 12476671 |
| chr16 | 15263834 | 15263884 |
| chr16 | 15623946 | 15623996 |
| chr16 | 16553085 | 16553135 |
| chr16 | 18118433 | 18118483 |
| chr16 | 22644299 | 22644349 |
| chr16 | 22714231 | 22714281 |
| chr16 | 22942215 | 22942265 |
| chr16 | 26559308 | 26559358 |
| chr16 | 30203275 | 30203325 |
| chr16 | 46677611 | 46677661 |
| chr16 | 50308674 | 50308724 |
| chr16 | 53156359 | 53156409 |
| chr16 | 54108194 | 54108244 |
| chr16 | 54427736 | 54427786 |
| chr10 | 73260659 | 73260709 |
| chr10 | 73315096 | 73315146 |
| chr16 | 59018412 | 59018462 |
| chr16 | 59056896 | 59056946 |
| chr16 | 81209664 | 81209714 |
| chr16 | 85443893 | 85443943 |
| chr16 | 86967473 | 86967523 |
| chr16 | 87615363 | 87615413 |
| chr16 | 87994456 | 87994506 |
| chr16 | 89434528 | 89434578 |
| chr17 | 3810475 | 3810525 |
| chr17 | 6145797 | 6145847 |
| chr17 | 6956749 | 6956799 |
| chr17 | 6957302 | 6957352 |
| chr17 | 8879079 | 8879129 |
| chr17 | 12646700 | 12646750 |
| chr17 | 14566587 | 14566637 |
| chr17 | 15229635 | 15229685 |
| chr17 | 16684482 | 16684532 |
| chr17 | 17760955 | 17761005 |
| chr17 | 35714619 | 35714669 |

Table 15DQ. Repeat Category MSTB-int

| | | |
|---|---|---|
| chr17 | 48567557 | 48567607 |
| chr17 | 54421646 | 54421696 |
| chr18 | 61887606 | 61887656 |
| chr1 | 46839776 | 46839826 |
| chr11 | 4786972 | 4787022 |
| chrX | 71612683 | 71612733 |
| chrX | 86766044 | 86766094 |
| chrX | 93600896 | 93600946 |
| chr2 | 113642954 | 113643004 |
| chr3 | 147400213 | 147400263 |
| chr4 | 18575341 | 18575391 |
| chr4 | 47575168 | 47575218 |
| chr4 | 124762191 | 124762241 |
| chr4 | 126541857 | 126541907 |
| chr4 | 190931367 | 190931417 |
| chr5 | 98686461 | 98686511 |
| chr11 | 71946042 | 71946092 |
| chr6 | 14695949 | 14695999 |
| chr6 | 76474844 | 76474894 |
| chr6 | 86022358 | 86022408 |
| chr6 | 126457377 | 126457427 |
| chr7 | 101473407 | 101473457 |
| chr11 | 123170295 | 123170345 |
| chr8 | 77022855 | 77022905 |
| chr8 | 144252762 | 144252812 |
| chr8 | 144252687 | 144252737 |
| chr11 | 134025879 | 134025929 |
| chr9 | 120975059 | 120975109 |
| chr12 | 21238279 | 21238329 |
| chr13 | 39076798 | 39076848 |
| chr13 | 74499453 | 74499503 |
| chr14 | 66800897 | 66800947 |
| chr14 | 104880917 | 104880967 |
| chr15 | 90837864 | 90837914 |

Table 15DR. Repeat Category MSTB1

| | | |
|---|---|---|
| chr10 | 82267400 | 82267450 |
| chr17 | 55351216 | 55351266 |
| chr17 | 57505478 | 57505528 |
| chr17 | 67741387 | 67741437 |
| chr17 | 70585186 | 70585236 |
| chr17 | 80912139 | 80912189 |
| chr17 | 80913287 | 80913337 |
| chr17 | 81608104 | 81608154 |
| chr18 | 2701766 | 2701816 |
| chr18 | 29626733 | 29626783 |
| chr18 | 46096167 | 46096217 |
| chr18 | 49314504 | 49314554 |
| chr18 | 58320407 | 58320457 |
| chr18 | 69950420 | 69950470 |
| chr19 | 1656176 | 1656226 |
| chr19 | 6087882 | 6087932 |
| chr19 | 10987160 | 10987210 |
| chr19 | 17473222 | 17473272 |
| chr19 | 33636370 | 33636420 |
| chr10 | 105192813 | 105192863 |
| chr10 | 105193243 | 105193293 |
| chr10 | 105193529 | 105193579 |
| chr19 | 53296313 | 53296363 |
| chr19 | 56327657 | 56327707 |
| chr1 | 5268962 | 5269012 |
| chr1 | 13647332 | 13647382 |
| chr1 | 14216346 | 14216396 |
| chr1 | 26126487 | 26126537 |
| chr1 | 57886437 | 57886487 |
| chr1 | 58028929 | 58028979 |
| chr1 | 58989326 | 58989376 |
| chr1 | 90255661 | 90255711 |
| chr10 | 125379054 | 125379104 |
| chr10 | 125378842 | 125378892 |
| chr1 | 108556221 | 108556271 |
| chr1 | 164029617 | 164029667 |
| chr1 | 173130455 | 173130505 |
| chr1 | 191578364 | 191578414 |
| chr1 | 225213113 | 225213163 |
| chr1 | 225589050 | 225589100 |
| chr1 | 227455774 | 227455824 |
| chr1 | 231470261 | 231470311 |
| chr1 | 240132156 | 240132206 |
| chr20 | 31502272 | 31502322 |
| chr10 | 134609524 | 134609574 |
| chr20 | 63479487 | 63479537 |
| chr21 | 16014761 | 16014811 |
| chr21 | 32601489 | 32601539 |
| chr21 | 33956644 | 33956694 |
| chr21 | 40849941 | 40849991 |
| chr21 | 42822247 | 42822297 |
| chr22 | 16104214 | 16104264 |
| chr22 | 16526760 | 16526810 |
| chr22 | 18408047 | 18408097 |
| chr22 | 41846210 | 41846260 |
| chrX | 15071533 | 15071583 |
| chrX | 23832987 | 23833037 |
| chrX | 34433193 | 34433243 |
| chrX | 48016926 | 48016976 |
| chrX | 50608314 | 50608364 |
| chrX | 53083196 | 53083246 |
| chrX | 81863466 | 81863516 |
| chrX | 117231013 | 117231063 |
| chrX | 117654850 | 117654900 |
| chrX | 131646562 | 131646612 |
| chrX | 140212967 | 140213017 |
| chrY | 6122806 | 6122856 |
| chr2 | 3178777 | 3178827 |
| chr2 | 30408202 | 30408252 |
| chr2 | 61969776 | 61969826 |
| chr11 | 25574739 | 25574789 |
| chr11 | 25574558 | 25574608 |
| chr2 | 85903251 | 85903301 |
| chr2 | 98044846 | 98044896 |
| chr2 | 115439544 | 115439594 |
| chr2 | 121437900 | 121437950 |
| chr2 | 129438915 | 129438965 |
| chr2 | 138594488 | 138594538 |
| chr2 | 143495291 | 143495341 |
| chr2 | 151718328 | 151718378 |
| chr2 | 202178389 | 202178439 |
| chr2 | 202178325 | 202178375 |
| chr2 | 203129931 | 203129981 |
| chr2 | 216982642 | 216982692 |
| chr2 | 231957341 | 231957391 |
| chr11 | 45801643 | 45801693 |
| chr2 | 241454760 | 241454810 |
| chr2 | 241844409 | 241844459 |
| chr3 | 16491880 | 16491930 |
| chr3 | 16491653 | 16491703 |
| chr3 | 17923921 | 17923971 |
| chr3 | 41847584 | 41847634 |
| chr3 | 45140512 | 45140562 |
| chr3 | 49814426 | 49814476 |
| chr3 | 80430511 | 80430561 |
| chr3 | 88176564 | 88176614 |
| chr3 | 158176367 | 158176417 |
| chr3 | 176081746 | 176081796 |
| chr3 | 186422313 | 186422363 |
| chr4 | 11753452 | 11753502 |
| chr4 | 17136944 | 17136994 |
| chr4 | 67868869 | 67868919 |
| chr4 | 72208133 | 72208183 |
| chr4 | 119965655 | 119965705 |
| chr4 | 179237751 | 179237801 |
| chr4 | 181203613 | 181203663 |
| chr4 | 185935433 | 185935483 |
| chr5 | 767762 | 767812 |
| chr5 | 852060 | 852110 |
| chr5 | 3297205 | 3297255 |
| chr5 | 16962395 | 16962445 |
| chr5 | 20852747 | 20852797 |
| chr5 | 26979439 | 26979489 |
| chr5 | 34442340 | 34442390 |
| chr11 | 70015849 | 70015899 |
| chr5 | 55084043 | 55084093 |
| chr5 | 62116687 | 62116737 |
| chr5 | 62116753 | 62116803 |
| chr5 | 67129551 | 67129601 |
| chr5 | 171284437 | 171284487 |
| chr5 | 171284271 | 171284321 |
| chr5 | 175848410 | 175848460 |
| chr11 | 75898361 | 75898411 |
| chr5 | 177509254 | 177509304 |
| chr6 | 17466319 | 17466369 |
| chr6 | 22642952 | 22643002 |
| chr6 | 23679967 | 23680017 |
| chr6 | 27446473 | 27446523 |
| chr6 | 49510392 | 49510442 |
| chr11 | 88237218 | 88237268 |
| chr11 | 89684981 | 89685031 |
| chr6 | 74398785 | 74398835 |
| chr11 | 92325486 | 92325536 |
| chr6 | 90333708 | 90333758 |
| chr6 | 103686484 | 103686534 |
| chr6 | 116960864 | 116960914 |
| chr6 | 124817572 | 124817622 |
| chr6 | 124817407 | 124817457 |
| chr6 | 134431790 | 134431840 |
| chr6 | 143743612 | 143743662 |
| chr6 | 144578458 | 144578508 |
| chr11 | 100825792 | 100825842 |
| chr6 | 161345981 | 161346031 |
| chr6 | 166012383 | 166012433 |
| chr7 | 1648753 | 1648803 |
| chr7 | 1916827 | 1916877 |
| chr7 | 3687404 | 3687454 |
| chr7 | 15545104 | 15545154 |
| chr7 | 21742240 | 21742290 |
| chr7 | 30166593 | 30166643 |
| chr7 | 30166673 | 30166723 |
| chr7 | 44451749 | 44451799 |
| chr7 | 70119658 | 70119708 |
| chr7 | 71026673 | 71026723 |
| chr7 | 78741810 | 78741860 |
| chr7 | 97475261 | 97475311 |
| chr7 | 150986282 | 150986332 |
| chr8 | 6147929 | 6147979 |
| chr8 | 16570215 | 16570265 |
| chr8 | 21283134 | 21283184 |
| chr10 | 18536301 | 18536351 |
| chr8 | 32734976 | 32735026 |
| chr8 | 37825367 | 37825417 |
| chr8 | 39346162 | 39346212 |
| chr8 | 49718688 | 49718738 |
| chr8 | 55790827 | 55790877 |
| chr8 | 76541573 | 76541623 |
| chr8 | 83576394 | 83576444 |
| chr8 | 98717863 | 98717913 |
| chr8 | 137294881 | 137294931 |
| chr11 | 130929079 | 130929129 |
| chr9 | 6580065 | 6580115 |
| chr9 | 81492799 | 81492849 |
| chr9 | 82987343 | 82987393 |
| chr9 | 133167436 | 133167486 |
| chr12 | 4154188 | 4154238 |
| chr12 | 4207661 | 4207711 |
| chr10 | 22870670 | 22870720 |
| chr10 | 24039681 | 24039731 |
| chr10 | 24057720 | 24057770 |
| chr12 | 34254285 | 34254335 |
| chr12 | 76921358 | 76921408 |
| chr12 | 78325177 | 78325227 |
| chr12 | 102853073 | 102853123 |
| chr12 | 115191144 | 115191194 |
| chr10 | 31447112 | 31447162 |
| chr12 | 116927846 | 116927896 |
| chr12 | 129609979 | 129610029 |
| chr13 | 18249389 | 18249439 |
| chr13 | 29447391 | 29447441 |
| chr13 | 31917515 | 31917565 |
| chr13 | 50262066 | 50262116 |
| chr13 | 53462193 | 53462243 |
| chr13 | 53840773 | 53840823 |
| chr13 | 59800054 | 59800104 |
| chr13 | 72012773 | 72012823 |
| chr13 | 76022076 | 76022126 |
| chr10 | 43522029 | 43522079 |
| chr14 | 32552191 | 32552241 |
| chr14 | 49632226 | 49632276 |
| chr10 | 50861784 | 50861834 |
| chr14 | 73514365 | 73514415 |
| chr14 | 73515627 | 73515677 |
| chr10 | 52923500 | 52923550 |
| chr14 | 79785522 | 79785572 |
| chr14 | 84851761 | 84851811 |
| chr14 | 98418533 | 98418583 |
| chr15 | 21727893 | 21727943 |
| chr15 | 53356617 | 53356667 |
| chr15 | 72919310 | 72919360 |
| chr15 | 72921328 | 72921378 |
| chr15 | 74756056 | 74756106 |
| chr16 | 1803005 | 1803055 |
| chr16 | 15263834 | 15263884 |
| chr16 | 15623946 | 15623996 |
| chr16 | 16553085 | 16553135 |
| chr16 | 22714231 | 22714281 |
| chr16 | 22942215 | 22942265 |
| chr16 | 46677611 | 46677661 |
| chr16 | 53156359 | 53156409 |
| chr16 | 87994456 | 87994506 |
| chr16 | 89434528 | 89434578 |
| chr17 | 8879079 | 8879129 |
| chr17 | 16684482 | 16684532 |

Table 15DS. Repeat Category MSTC

| | | |
|---|---|---|
| chr17 | 49223574 | 49223624 |
| chr17 | 58265918 | 58265968 |
| chr17 | 75176876 | 75176926 |
| chr17 | 77265523 | 77265573 |
| chr18 | 6319433 | 6319483 |
| chr18 | 7935504 | 7935554 |
| chr18 | 7935063 | 7935113 |
| chr10 | 94645208 | 94645258 |
| chr18 | 39054226 | 39054276 |
| chr18 | 45254771 | 45254821 |
| chr18 | 47025108 | 47025158 |
| chr19 | 2752743 | 2752793 |
| chr19 | 7996493 | 7996543 |
| chr19 | 51224142 | 51224192 |
| chr10 | 112101943 | 112101993 |
| chr19 | 59229744 | 59229794 |
| chr19 | 62654555 | 62654605 |
| chr1 | 20160557 | 20160607 |
| chr1 | 23841184 | 23841234 |
| chr1 | 39857947 | 39857997 |
| chr1 | 45267386 | 45267436 |
| chr1 | 46209492 | 46209542 |
| chr1 | 87428705 | 87428755 |
| chr1 | 109279029 | 109279079 |
| chr1 | 112843013 | 112843063 |
| chr1 | 114419057 | 114419107 |
| chr10 | 127358135 | 127358185 |
| chr1 | 180554469 | 180554519 |
| chr1 | 184956525 | 184956575 |
| chr1 | 198067310 | 198067360 |
| chr1 | 205954407 | 205954457 |
| chr1 | 215685991 | 215686041 |
| chr1 | 228043173 | 228043223 |
| chr10 | 131829430 | 131829480 |
| chr1 | 244076008 | 244076058 |
| chr1 | 244426531 | 244426581 |
| chr20 | 4027071 | 4027121 |
| chr20 | 4306659 | 4306709 |
| chr20 | 5197893 | 5197943 |
| chr20 | 25353621 | 25353671 |
| chr20 | 31707957 | 31708007 |
| chr10 | 7930179 | 7930229 |
| chr20 | 50683379 | 50683429 |
| chr20 | 57196000 | 57196050 |
| chr21 | 15101931 | 15101981 |
| chr21 | 22921165 | 22921215 |
| chr11 | 869773 | 869823 |
| chr22 | 16526760 | 16526810 |
| chr22 | 19048340 | 19048390 |
| chr22 | 25002284 | 25002334 |
| chr22 | 29740400 | 29740450 |
| chr22 | 37902423 | 37902473 |
| chr22 | 40095366 | 40095416 |
| chr22 | 42379202 | 42379252 |
| chr22 | 42875195 | 42875245 |
| chr22 | 48955466 | 48955516 |
| chrX | 39753937 | 39753987 |
| chrX | 46738835 | 46738885 |
| chrX | 53093966 | 53094016 |
| chrX | 53103070 | 53103120 |
| chrX | 61386426 | 61386476 |
| chrX | 62045450 | 62045500 |
| chrX | 117223119 | 117223169 |
| chrX | 138756298 | 138756348 |
| chrX | 153122807 | 153122857 |
| chr2 | 3584474 | 3584524 |
| chr2 | 9324308 | 9324358 |
| chr2 | 10423470 | 10423520 |
| chr2 | 21012226 | 21012276 |
| chr2 | 38035945 | 38035995 |
| chr2 | 63841612 | 63841662 |
| chr2 | 65844411 | 65844461 |
| chr2 | 66263669 | 66263719 |
| chr2 | 83210236 | 83210286 |
| chr2 | 101679313 | 101679363 |
| chr2 | 104127966 | 104128016 |
| chr2 | 127723082 | 127723132 |
| chr11 | 33178388 | 33178438 |
| chr2 | 179321318 | 179321368 |
| chr2 | 218715874 | 218715924 |
| chr2 | 240591870 | 240591920 |
| chr3 | 28804440 | 28804490 |
| chr3 | 86523315 | 86523365 |
| chr3 | 107085198 | 107085248 |
| chr3 | 121011643 | 121011693 |
| chr3 | 127060492 | 127060542 |
| chr3 | 154271240 | 154271290 |
| chr3 | 175465669 | 175465719 |
| chr3 | 178139841 | 178139891 |
| chr3 | 188246468 | 188246518 |
| chr3 | 188246264 | 188246314 |
| chr11 | 63115209 | 63115259 |
| chr4 | 36418577 | 36418627 |
| chr4 | 74383772 | 74383822 |
| chr4 | 84413645 | 84413695 |
| chr11 | 65992489 | 65992539 |
| chr4 | 102355966 | 102356016 |
| chr4 | 134128027 | 134128077 |
| chr4 | 139195747 | 139195797 |
| chr4 | 149209592 | 149209642 |
| chr4 | 179545333 | 179545383 |
| chr5 | 26997896 | 26997946 |
| chr5 | 40506098 | 40506148 |
| chr5 | 42221006 | 42221056 |
| chr5 | 42220756 | 42220806 |
| chr5 | 53381788 | 53381838 |
| chr5 | 55363659 | 55363709 |
| chr11 | 70600025 | 70600075 |
| chr5 | 74443459 | 74443509 |
| chr5 | 118871576 | 118871626 |
| chr5 | 129990024 | 129990074 |
| chr5 | 165683010 | 165683060 |
| chr5 | 168745591 | 168745641 |
| chr5 | 180025883 | 180025933 |
| chr6 | 393123 | 393173 |
| chr6 | 6744907 | 6744957 |
| chr11 | 78349472 | 78349522 |
| chr6 | 30567379 | 30567429 |
| chr6 | 32400938 | 32400988 |
| chr6 | 42725398 | 42725448 |
| chr6 | 146448076 | 146448126 |
| chr6 | 155240929 | 155240979 |
| chr6 | 158725212 | 158725262 |
| chr6 | 161760821 | 161760871 |
| chr11 | 107236880 | 107236930 |
| chr7 | 4317485 | 4317535 |
| chr7 | 5510871 | 5510921 |
| chr7 | 7011368 | 7011418 |
| chr7 | 19142316 | 19142366 |
| chr7 | 69816616 | 69816666 |
| chr7 | 69916233 | 69916283 |
| chr7 | 86231719 | 86231769 |
| chr7 | 90239855 | 90239905 |
| chr7 | 96288395 | 96288445 |
| chr11 | 117950493 | 117950543 |
| chr7 | 147377214 | 147377264 |
| chr7 | 152250288 | 152250338 |
| chr7 | 156441007 | 156441057 |
| chr7 | 156440791 | 156440841 |
| chr8 | 23068626 | 23068676 |
| chr8 | 81352205 | 81352255 |
| chr8 | 131891567 | 131891617 |
| chr8 | 144846403 | 144846453 |
| chr9 | 8900388 | 8900438 |
| chr9 | 34663243 | 34663293 |
| chr9 | 37744942 | 37744992 |
| chr9 | 67112392 | 67112442 |
| chr9 | 71076714 | 71076764 |
| chr9 | 85796398 | 85796448 |
| chr9 | 111351760 | 111351810 |
| chr9 | 130991079 | 130991129 |
| chr9 | 135214605 | 135214655 |
| chr12 | 7999618 | 7999668 |
| chr12 | 31945285 | 31945335 |
| chr12 | 39575608 | 39575658 |
| chr12 | 52320280 | 52320330 |
| chr12 | 52321612 | 52321662 |
| chr12 | 100325057 | 100325107 |
| chr12 | 111483638 | 111483688 |
| chr12 | 115418466 | 115418516 |
| chr12 | 131850894 | 131850944 |
| chr13 | 17910936 | 17910986 |
| chr13 | 39573871 | 39573921 |
| chr13 | 60786063 | 60786113 |
| chr13 | 76746786 | 76746836 |
| chr13 | 87557235 | 87557285 |
| chr14 | 70028797 | 70028847 |
| chr14 | 71827127 | 71827177 |
| chr14 | 79377960 | 79378010 |
| chr14 | 84341952 | 84342002 |
| chr14 | 87918997 | 87919047 |
| chr14 | 90393525 | 90393575 |
| chr14 | 94832153 | 94832203 |
| chr14 | 98642422 | 98642472 |
| chr14 | 98645152 | 98645202 |
| chr14 | 100170221 | 100170271 |
| chr14 | 100170284 | 100170334 |
| chr15 | 22095918 | 22095968 |
| chr15 | 22386634 | 22386684 |
| chr10 | 63625136 | 63625186 |
| chr10 | 63624530 | 63624580 |
| chr15 | 61988772 | 61988822 |
| chr15 | 75075059 | 75075109 |
| chr15 | 85849930 | 85849980 |
| chr15 | 85849863 | 85849913 |
| chr15 | 87852749 | 87852799 |
| chr15 | 98876768 | 98876818 |
| chr15 | 99236593 | 99236643 |
| chr16 | 136066 | 136116 |
| chr16 | 413009 | 413059 |
| chr10 | 70280865 | 70280915 |
| chr16 | 2210094 | 2210144 |
| chr16 | 9210344 | 9210394 |
| chr16 | 12169904 | 12169954 |
| chr16 | 19879657 | 19879707 |
| chr16 | 25794237 | 25794287 |
| chr16 | 66343915 | 66343965 |
| chr16 | 75476527 | 75476577 |
| chr16 | 75575684 | 75575734 |
| chr16 | 83831461 | 83831511 |
| chr10 | 3319173 | 3319223 |
| chr16 | 87359110 | 87359160 |
| chr10 | 76012564 | 76012614 |
| chr10 | 76421584 | 76421634 |
| chr17 | 7135486 | 7135536 |
| chr17 | 7136669 | 7136719 |
| chr17 | 8642561 | 8642611 |
| chr17 | 9920205 | 9920255 |
| chr17 | 17739429 | 17739479 |
| chr17 | 20061485 | 20061535 |
| chr17 | 30815617 | 30815667 |

Table 15DT. Repeat Category MSTD

| | | |
|---|---|---|
| chr17 | 43738723 | 43738773 |
| chr17 | 45532986 | 45533036 |
| chr17 | 49348289 | 49348339 |
| chr17 | 50076545 | 50076595 |
| chr17 | 64354869 | 64354919 |
| chr17 | 70447280 | 70447330 |
| chr10 | 91061511 | 91061561 |
| chr17 | 79431375 | 79431425 |
| chr17 | 79431253 | 79431303 |
| chr18 | 30045015 | 30045065 |
| chr10 | 97806683 | 97806733 |
| chr18 | 58695927 | 58695977 |
| chr18 | 70339103 | 70339153 |
| chr19 | 1924561 | 1924611 |
| chr19 | 6746974 | 6747024 |
| chr19 | 18002336 | 18002386 |
| chr19 | 41423915 | 41423965 |
| chr19 | 45017121 | 45017171 |
| chr19 | 45017455 | 45017505 |
| chr19 | 45459348 | 45459398 |
| chr19 | 52412887 | 52412937 |
| chr19 | 52412992 | 52413042 |
| chr19 | 52413861 | 52413911 |
| chr19 | 52447915 | 52447965 |
| chr1 | 1066451 | 1066501 |
| chr1 | 3743795 | 3743845 |
| chr10 | 116234529 | 116234579 |
| chr1 | 7348055 | 7348105 |
| chr1 | 16729622 | 16729672 |
| chr1 | 20256799 | 20256849 |
| chr1 | 23730576 | 23730626 |
| chr1 | 30386744 | 30386794 |
| chr1 | 31843743 | 31843793 |
| chr1 | 39463055 | 39463105 |
| chr1 | 42683834 | 42683884 |
| chr1 | 45148590 | 45148640 |
| chr1 | 47193023 | 47193073 |
| chr1 | 65138075 | 65138125 |
| chr10 | 124312216 | 124312266 |
| chr10 | 124314004 | 124314054 |
| chr1 | 75888168 | 75888218 |
| chr1 | 80833521 | 80833571 |
| chr1 | 82407150 | 82407200 |
| chr1 | 99301370 | 99301420 |
| chr1 | 102470555 | 102470605 |
| chr1 | 102829815 | 102829865 |
| chr1 | 105056441 | 105056491 |
| chr1 | 141906328 | 141906378 |
| chr1 | 141906931 | 141906981 |
| chr1 | 162897100 | 162897150 |
| chr1 | 163667791 | 163667841 |
| chr1 | 170627883 | 170627933 |
| chr1 | 170627498 | 170627548 |
| chr1 | 173809339 | 173809389 |
| chr1 | 176279453 | 176279503 |
| chr1 | 179647472 | 179647522 |
| chr1 | 197716439 | 197716489 |
| chr1 | 214539452 | 214539502 |
| chr1 | 239772333 | 239772383 |
| chr10 | 132297213 | 132297263 |
| chr1 | 243449738 | 243449788 |
| chr20 | 4030829 | 4030879 |
| chr20 | 34729575 | 34729625 |
| chr20 | 53115309 | 53115359 |
| chr20 | 54290571 | 54290621 |
| chr20 | 55725553 | 55725603 |
| chr20 | 62858940 | 62858990 |
| chr21 | 18106682 | 18106732 |
| chr21 | 18418060 | 18418110 |
| chr21 | 24273192 | 24273242 |
| chr21 | 35119635 | 35119685 |
| chr21 | 35119017 | 35119067 |
| chr21 | 43466253 | 43466303 |
| chr21 | 43714375 | 43714425 |
| chr22 | 31779866 | 31779916 |
| chr22 | 37307168 | 37307218 |
| chr22 | 43770699 | 43770749 |
| chr22 | 47403855 | 47403905 |
| chr22 | 48220341 | 48220391 |
| chrX | 5462786 | 5462836 |
| chrX | 12142713 | 12142763 |
| chrX | 23795630 | 23795680 |
| chrX | 24665184 | 24665234 |
| chrX | 30385238 | 30385288 |
| chrX | 36851284 | 36851334 |
| chrX | 40037922 | 40037972 |
| chrX | 42899401 | 42899451 |
| chrX | 47992094 | 47992144 |
| chrX | 54365148 | 54365198 |
| chr11 | 8266660 | 8266710 |
| chrX | 81777222 | 81777272 |
| chrX | 85708031 | 85708081 |
| chrX | 85714508 | 85714558 |
| chrX | 93239943 | 93239993 |
| chrX | 93290733 | 93290783 |
| chrX | 114334364 | 114334414 |
| chrX | 126583601 | 126583651 |
| chrX | 131058099 | 131058149 |
| chrX | 137638369 | 137638419 |
| chrX | 137645373 | 137645423 |
| chrX | 139612067 | 139612117 |
| chr11 | 12423796 | 12423846 |
| chrX | 150782133 | 150782183 |
| chrX | 150782676 | 150782726 |
| chrY | 3024715 | 3024765 |
| chrY | 21815090 | 21815140 |
| chr2 | 5620456 | 5620506 |
| chr11 | 17179388 | 17179438 |
| chr2 | 16116695 | 16116745 |
| chr2 | 19841006 | 19841056 |
| chr2 | 24057529 | 24057579 |
| chr2 | 39073747 | 39073797 |
| chr2 | 40153961 | 40154011 |
| chr2 | 63890153 | 63890203 |
| chr2 | 65316675 | 65316725 |
| chr2 | 70205880 | 70205930 |
| chr11 | 25814475 | 25814525 |
| chr2 | 72079481 | 72079531 |
| chr2 | 75499248 | 75499298 |
| chr2 | 83050040 | 83050090 |
| chr2 | 85423647 | 85423697 |
| chr2 | 91639134 | 91639184 |
| chr11 | 28610723 | 28610773 |
| chr2 | 98778873 | 98778923 |
| chr2 | 105590583 | 105590633 |
| chr11 | 31691064 | 31691114 |
| chr2 | 109170435 | 109170485 |
| chr2 | 113386749 | 113386799 |
| chr2 | 114666213 | 114666263 |
| chr2 | 138570986 | 138571036 |
| chr2 | 140775550 | 140775600 |
| chr2 | 149256417 | 149256467 |
| chr2 | 171293511 | 171293561 |
| chr2 | 172135643 | 172135693 |
| chr2 | 175674694 | 175674744 |
| chr2 | 179296325 | 179296375 |
| chr2 | 182319616 | 182319666 |
| chr2 | 186325045 | 186325095 |
| chr2 | 193697445 | 193697495 |
| chr2 | 194128026 | 194128076 |
| chr2 | 196564591 | 196564641 |
| chr2 | 219300124 | 219300174 |
| chr2 | 232042152 | 232042202 |
| chr2 | 241404409 | 241404459 |
| chr2 | 242786781 | 242786831 |
| chr3 | 3676706 | 3676756 |
| chr3 | 24001280 | 24001330 |
| chr3 | 33397439 | 33397489 |
| chr11 | 47749644 | 47749694 |
| chr3 | 60980262 | 60980312 |
| chr3 | 97520834 | 97520884 |
| chr3 | 104227089 | 104227139 |
| chr11 | 60127596 | 60127646 |
| chr3 | 132371115 | 132371165 |
| chr3 | 134209206 | 134209256 |
| chr3 | 148009254 | 148009304 |
| chr3 | 161218343 | 161218393 |
| chr3 | 171217151 | 171217201 |
| chr3 | 173356286 | 173356336 |
| chr3 | 173912627 | 173912677 |
| chr3 | 178655690 | 178655740 |
| chr11 | 62262407 | 62262457 |
| chr4 | 2806392 | 2806442 |
| chr4 | 3722738 | 3722788 |
| chr4 | 5900462 | 5900512 |
| chr4 | 6662281 | 6662331 |
| chr4 | 21267829 | 21267879 |
| chr4 | 28801115 | 28801165 |
| chr4 | 38320725 | 38320775 |
| chr4 | 65103738 | 65103788 |
| chr4 | 67072324 | 67072374 |
| chr4 | 69226257 | 69226307 |
| chr4 | 103542545 | 103542595 |
| chr4 | 111032984 | 111033034 |
| chr4 | 111057630 | 111057680 |
| chr4 | 111059573 | 111059623 |
| chr4 | 115757127 | 115757177 |
| chr4 | 125274904 | 125274954 |
| chr4 | 138040067 | 138040117 |
| chr4 | 140254634 | 140254684 |
| chr4 | 146976983 | 146977033 |
| chr4 | 149148078 | 149148128 |
| chr4 | 153514868 | 153514918 |
| chr4 | 183461044 | 183461094 |
| chr4 | 183461165 | 183461215 |
| chr4 | 183461415 | 183461465 |
| chr4 | 184706031 | 184706081 |
| chr4 | 186732212 | 186732262 |
| chr5 | 1615936 | 1615986 |
| chr5 | 12822453 | 12822503 |
| chr5 | 15131992 | 15132042 |
| chr5 | 21578232 | 21578282 |
| chr5 | 22148192 | 22148242 |
| chr5 | 22632364 | 22632414 |
| chr5 | 23559092 | 23559142 |
| chr5 | 33331587 | 33331637 |
| chr5 | 58993631 | 58993681 |
| chr5 | 64848896 | 64848946 |
| chr5 | 66499603 | 66499653 |
| chr5 | 72899463 | 72899513 |
| chr11 | 70960369 | 70960419 |
| chr11 | 71022050 | 71022100 |
| chr5 | 126895014 | 126895064 |
| chr5 | 131931958 | 131932008 |
| chr11 | 72907546 | 72907596 |
| chr5 | 138360702 | 138360752 |
| chr5 | 142886871 | 142886921 |
| chr5 | 148886376 | 148886426 |
| chr5 | 157324900 | 157324950 |
| chr5 | 157338016 | 157338066 |
| chr5 | 161079161 | 161079211 |
| chr5 | 165621557 | 165621607 |
| chr5 | 171447268 | 171447318 |
| chr5 | 173652167 | 173652217 |
| chr6 | 7320875 | 7320925 |
| chr6 | 10215228 | 10215278 |
| chr6 | 13678216 | 13678266 |
| chr6 | 15609887 | 15609937 |
| chr6 | 17450880 | 17450930 |
| chr6 | 23793761 | 23793811 |
| chr6 | 30567379 | 30567429 |
| chr6 | 37058334 | 37058384 |
| chr6 | 37127691 | 37127741 |
| chr6 | 71774224 | 71774274 |
| chr6 | 72350694 | 72350744 |
| chr6 | 80935768 | 80935818 |
| chr6 | 107256215 | 107256265 |
| chr6 | 110966109 | 110966159 |
| chr6 | 134445871 | 134445921 |
| chr6 | 140025662 | 140025712 |
| chr6 | 142018072 | 142018122 |
| chr6 | 144560003 | 144560053 |
| chr6 | 153311845 | 153311895 |
| chr6 | 159933524 | 159933574 |
| chr6 | 159933845 | 159933895 |
| chr6 | 161256490 | 161256540 |
| chr6 | 162181882 | 162181932 |
| chr6 | 170253920 | 170253970 |
| chr6 | 170322679 | 170322729 |
| chr6 | 170322289 | 170322339 |
| chr7 | 15934339 | 15934389 |
| chr7 | 32872814 | 32872864 |
| chr7 | 35094830 | 35094880 |
| chr7 | 36533292 | 36533342 |
| chr7 | 63899329 | 63899379 |
| chr7 | 81779845 | 81779895 |
| chr7 | 88609021 | 88609071 |
| chr10 | 971772 | 971822 |
| chr11 | 117478034 | 117478084 |
| chr7 | 129260636 | 129260686 |
| chr7 | 134646503 | 134646553 |
| chr7 | 142192010 | 142192060 |
| chr7 | 146308980 | 146309030 |
| chr7 | 148777194 | 148777244 |
| chr7 | 149016006 | 149016056 |
| chr8 | 13242512 | 13242562 |
| chr8 | 13686403 | 13686453 |
| chr8 | 30242500 | 30242550 |
| chr8 | 33549778 | 33549828 |
| chr8 | 58204850 | 58204900 |
| chr11 | 125656200 | 125656250 |
| chr8 | 81181083 | 81181133 |
| chr8 | 83159889 | 83159939 |
| chr8 | 96317756 | 96317806 |
| chr8 | 98175453 | 98175503 |
| chr8 | 103769219 | 103769269 |
| chr10 | 18986048 | 18986098 |
| chr8 | 117930368 | 117930418 |
| chr8 | 124068231 | 124068281 |
| chr8 | 124147936 | 124147986 |
| chr8 | 124147788 | 124147838 |
| chr8 | 126658306 | 126658356 |
| chr8 | 142330104 | 142330154 |
| chr9 | 6236705 | 6236755 |
| chr9 | 17091118 | 17091168 |
| chr9 | 20251522 | 20251572 |
| chr9 | 25389724 | 25389774 |
| chr9 | 31262357 | 31262407 |
| chr9 | 33197992 | 33198042 |
| chr9 | 76776257 | 76776307 |
| chr9 | 83972525 | 83972575 |
| chr9 | 119232210 | 119232260 |
| chr9 | 123524649 | 123524699 |
| chr9 | 128152483 | 128152533 |
| chr9 | 129451181 | 129451231 |
| chr9 | 132293098 | 132293148 |
| chr12 | 2958931 | 2958981 |
| chr12 | 4378655 | 4378705 |
| chr12 | 6482883 | 6482933 |
| chr12 | 14398174 | 14398224 |
| chr12 | 17508576 | 17508626 |
| chr12 | 20300200 | 20300250 |
| chr12 | 25028702 | 25028752 |
| chr12 | 26943508 | 26943558 |
| chr12 | 43888213 | 43888263 |
| chr12 | 50841873 | 50841923 |
| chr10 | 27444500 | 27444550 |
| chr10 | 28117270 | 28117320 |
| chr10 | 28808079 | 28808129 |
| chr12 | 75813744 | 75813794 |
| chr12 | 100313075 | 100313125 |
| chr12 | 105956867 | 105956917 |
| chr12 | 118148833 | 118148883 |
| chr12 | 123110571 | 123110621 |
| chr12 | 131133988 | 131134038 |
| chr13 | 18609269 | 18609319 |
| chr13 | 23446581 | 23446631 |
| chr10 | 36703731 | 36703781 |
| chr13 | 25863440 | 25863490 |
| chr13 | 31402114 | 31402164 |
| chr13 | 52707833 | 52707883 |
| chr13 | 61389286 | 61389336 |
| chr13 | 73889015 | 73889065 |
| chr13 | 79671458 | 79671508 |
| chr13 | 111562838 | 111562888 |
| chr14 | 19251435 | 19251485 |
| chr14 | 24114849 | 24114899 |
| chr14 | 35751436 | 35751486 |
| chr14 | 43397426 | 43397476 |
| chr14 | 43397944 | 43397994 |
| chr14 | 43397539 | 43397589 |
| chr14 | 52928696 | 52928746 |
| chr14 | 54246808 | 54246858 |
| chr14 | 56371593 | 56371643 |
| chr14 | 59630337 | 59630387 |
| chr10 | 51476484 | 51476534 |
| chr14 | 68218035 | 68218085 |
| chr14 | 73918164 | 73918214 |
| chr14 | 91213061 | 91213111 |
| chr14 | 100619643 | 100619693 |
| chr14 | 101031609 | 101031659 |
| chr14 | 103973354 | 103973404 |
| chr15 | 23066720 | 23066770 |
| chr15 | 27248673 | 27248723 |
| chr15 | 33231483 | 33231533 |
| chr15 | 43752861 | 43752911 |
| chr15 | 48501306 | 48501356 |
| chr15 | 51200865 | 51200915 |
| chr15 | 58424712 | 58424762 |
| chr15 | 62949040 | 62949090 |
| chr15 | 64957485 | 64957535 |
| chr15 | 72508130 | 72508180 |
| chr15 | 81315284 | 81315334 |
| chr15 | 81314781 | 81314831 |
| chr15 | 99264746 | 99264796 |
| chr15 | 99264648 | 99264698 |
| chr10 | 70285607 | 70285657 |
| chr16 | 7248087 | 7248137 |
| chr10 | 3051755 | 3051805 |
| chr10 | 71616565 | 71616615 |
| chr16 | 20906968 | 20907018 |
| chr16 | 29063507 | 29063557 |
| chr16 | 35674914 | 35674964 |
| chr16 | 68527548 | 68527598 |
| chr16 | 69591980 | 69592030 |
| chr16 | 74628452 | 74628502 |
| chr16 | 87776616 | 87776666 |
| chr16 | 89880459 | 89880509 |
| chr17 | 276139 | 276189 |
| chr17 | 552917 | 552967 |
| chr17 | 16700384 | 16700434 |
| chr17 | 19030795 | 19030845 |
| chr17 | 19762274 | 19762324 |
| chr17 | 21559575 | 21559625 |
| chr17 | 30815617 | 30815667 |

Table 15DU. Repeat Category PABL_A

| | | |
|---|---|---|
| chr17 | 72568231 | 72568281 |
| chr17 | 77261469 | 77261519 |
| chr17 | 80981555 | 80981605 |
| chr18 | 75923806 | 75923856 |
| chr10 | 99786992 | 99787042 |
| chr19 | 19670682 | 19670732 |
| chr19 | 34809321 | 34809371 |
| chr19 | 56442846 | 56442896 |
| chr1 | 39801573 | 39801623 |
| chr1 | 143790728 | 143790778 |
| chr1 | 231283070 | 231283120 |
| chrX | 2455769 | 2455819 |
| chrX | 6724490 | 6724540 |
| chrX | 16108953 | 16109003 |
| chrX | 16112159 | 16112209 |
| chrX | 16588750 | 16588800 |
| chrX | 68159818 | 68159868 |
| chrX | 109566684 | 109566734 |
| chrX | 109686451 | 109686501 |
| chrX | 124825702 | 124825752 |
| chrX | 124829612 | 124829662 |
| chrY | 4927929 | 4927979 |
| chr2 | 133451 | 133501 |
| chr2 | 30283438 | 30283488 |
| chr2 | 127848432 | 127848482 |
| chr2 | 243070936 | 243070986 |
| chr3 | 3144325 | 3144375 |
| chr3 | 3144439 | 3144489 |
| chr3 | 3145469 | 3145519 |
| chr3 | 46960831 | 46960881 |
| chr3 | 123509622 | 123509672 |
| chr3 | 129710293 | 129710343 |
| chr4 | 142212094 | 142212144 |
| chr4 | 164060926 | 164060976 |
| chr4 | 164061119 | 164061169 |
| chr4 | 164061270 | 164061320 |
| chr4 | 164061519 | 164061569 |
| chr11 | 71588207 | 71588257 |
| chr6 | 107368702 | 107368752 |
| chr11 | 97480148 | 97480198 |
| chr6 | 169495542 | 169495592 |
| chr7 | 34532025 | 34532075 |
| chr7 | 34533818 | 34533868 |
| chr7 | 57345133 | 57345183 |
| chr8 | 22932018 | 22932068 |
| chr8 | 23119513 | 23119563 |
| chr8 | 23121541 | 23121591 |
| chr8 | 26089194 | 26089244 |
| chr9 | 92370890 | 92370940 |
| chr9 | 136045910 | 136045960 |
| chr12 | 48662383 | 48662433 |
| chr12 | 51791449 | 51791499 |
| chr13 | 21255392 | 21255442 |
| chr13 | 48064778 | 48064828 |
| chr14 | 19468645 | 19468695 |
| chr10 | 59239398 | 59239448 |
| chr15 | 20006983 | 20007033 |
| chr16 | 2249820 | 2249870 |
| chr16 | 2252152 | 2252202 |
| chr16 | 10342661 | 10342711 |
| chr16 | 12378743 | 12378793 |
| chr16 | 84020451 | 84020501 |

Table 15DV. Repeat Category PABL_B

| | | |
|---|---|---|
| chr10 | 85531577 | 85531627 |
| chr18 | 46230633 | 46230683 |
| chr18 | 63151271 | 63151321 |
| chr19 | 61162672 | 61162722 |
| chr1 | 16907790 | 16907840 |
| chr21 | 33705168 | 33705218 |
| chr22 | 48266608 | 48266658 |
| chrX | 2451150 | 2451200 |
| chr11 | 5624905 | 5624955 |
| chrX | 92036559 | 92036609 |
| chrX | 95869474 | 95869524 |
| chrX | 95873751 | 95873801 |
| chrX | 129543009 | 129543059 |
| chrY | 13469834 | 13469884 |
| chr2 | 30191351 | 30191401 |
| chr2 | 54232197 | 54232247 |
| chr3 | 42806657 | 42806707 |
| chr3 | 42807416 | 42807466 |
| chr4 | 103542545 | 103542595 |
| chr4 | 188045924 | 188045974 |
| chr4 | 188045864 | 188045914 |
| chr6 | 26424403 | 26424453 |
| chr6 | 32179199 | 32179249 |
| chr6 | 53481562 | 53481612 |
| chr11 | 107188156 | 107188206 |
| chr7 | 4799187 | 4799237 |
| chr7 | 134425891 | 134425941 |
| chr8 | 54315147 | 54315197 |
| chr8 | 144252687 | 144252737 |
| chr9 | 133981791 | 133981841 |
| chr9 | 133990061 | 133990111 |
| chr9 | 133991258 | 133991308 |
| chr9 | 133994121 | 133994171 |
| chr12 | 30563067 | 30563117 |
| chr16 | 9305240 | 9305290 |
| chr17 | 11013127 | 11013177 |

Table 15DW. Repeat Category SST1

| | | |
|---|---|---|
| chr18 | 11179136 | 11179186 |
| chr19 | 41439574 | 41439624 |
| chr19 | 41442157 | 41442207 |
| chr19 | 41450941 | 41450991 |
| chr19 | 41454843 | 41454893 |
| chr19 | 42190779 | 42190829 |
| chr19 | 42192956 | 42193006 |
| chr19 | 42195250 | 42195300 |
| chr19 | 42197755 | 42197805 |
| chr19 | 42238521 | 42238571 |
| chr19 | 42240257 | 42240307 |
| chr19 | 42241761 | 42241811 |
| chr19 | 42243830 | 42243880 |
| chr19 | 42452388 | 42452438 |
| chr19 | 42464712 | 42464762 |
| chr19 | 42467493 | 42467543 |
| chr19 | 42487646 | 42487696 |
| chr19 | 42732769 | 42732819 |
| chr19 | 42734420 | 42734470 |
| chr19 | 42734152 | 42734202 |
| chr20 | 25829405 | 25829455 |
| chr20 | 25832323 | 25832373 |
| chr20 | 25834137 | 25834187 |
| chr20 | 25834860 | 25834910 |
| chr20 | 25836178 | 25836228 |
| chr20 | 25837963 | 25838013 |
| chr20 | 25839822 | 25839872 |
| chr20 | 25841510 | 25841560 |
| chr20 | 25843969 | 25844019 |
| chr20 | 25960261 | 25960311 |
| chr20 | 25961445 | 25961495 |
| chr20 | 25962189 | 25962239 |
| chr20 | 25962783 | 25962833 |
| chr20 | 25963950 | 25964000 |
| chr20 | 25965913 | 25965963 |
| chr20 | 25967555 | 25967605 |
| chr20 | 26227263 | 26227313 |
| chr20 | 29411493 | 29411543 |
| chr20 | 29413930 | 29413980 |
| chr20 | 29414479 | 29414529 |
| chr20 | 29416037 | 29416087 |
| chr20 | 29465353 | 29465403 |
| chr22 | 15603677 | 15603727 |
| chrY | 8244873 | 8244923 |
| chrY | 8586928 | 8586978 |
| chrY | 8594455 | 8594505 |
| chrY | 8599812 | 8599862 |
| chrY | 8802608 | 8802658 |
| chrY | 8832966 | 8833016 |
| chrY | 22603191 | 22603241 |
| chrY | 22604888 | 22604938 |
| chrY | 22612413 | 22612463 |
| chrY | 22811477 | 22811527 |
| chrY | 22816605 | 22816655 |
| chrY | 22816405 | 22816455 |
| chr4 | 133105673 | 133105723 |
| chr4 | 133108807 | 133108857 |
| chr4 | 133114432 | 133114482 |
| chr4 | 133119447 | 133119497 |
| chr4 | 133121931 | 133121981 |
| chr4 | 133128547 | 133128597 |
| chr4 | 133144611 | 133144661 |
| chr4 | 133356425 | 133356475 |
| chr6 | 2582761 | 2582811 |
| chr6 | 2584919 | 2584969 |
| chr6 | 2584618 | 2584668 |
| chr7 | 57323292 | 57323342 |
| chr7 | 57484576 | 57484626 |
| chr7 | 57489252 | 57489302 |
| chr7 | 57491668 | 57491718 |
| chr7 | 57493544 | 57493594 |
| chr7 | 57608476 | 57608526 |
| chr7 | 57611298 | 57611348 |
| chr7 | 57612957 | 57613007 |
| chr7 | 57614872 | 57614922 |
| chr7 | 57657592 | 57657642 |
| chr7 | 57658603 | 57658653 |
| chr7 | 57660994 | 57661044 |
| chr7 | 63740873 | 63740923 |
| chr7 | 63742146 | 63742196 |
| chr7 | 63742406 | 63742456 |
| chr7 | 63743064 | 63743114 |
| chr7 | 63745004 | 63745054 |
| chr7 | 63747477 | 63747527 |
| chr7 | 63750110 | 63750160 |
| chr7 | 63752506 | 63752556 |
| chr7 | 63755151 | 63755201 |
| chr7 | 63754991 | 63755041 |
| chr7 | 63756844 | 63756894 |
| chr9 | 42227834 | 42227884 |
| chr9 | 42278036 | 42278086 |
| chr9 | 42279994 | 42280044 |
| chr9 | 42281227 | 42281277 |
| chr9 | 42282424 | 42282474 |
| chr9 | 42283864 | 42283914 |
| chr9 | 42285059 | 42285109 |
| chr9 | 42286324 | 42286374 |
| chr9 | 43656896 | 43656946 |
| chr9 | 63427466 | 63427516 |
| chr9 | 63959998 | 63960048 |
| chr9 | 64968296 | 64968346 |
| chr12 | 36221876 | 36221926 |
| chr12 | 36223975 | 36224025 |
| chr12 | 36226413 | 36226463 |
| chr12 | 36227743 | 36227793 |
| chr16 | 35295200 | 35295250 |
| chr16 | 35456280 | 35456330 |
| chr16 | 35458498 | 35458548 |
| chr16 | 36108845 | 36108895 |
| chr16 | 36111212 | 36111262 |
| chr17 | 21639309 | 21639359 |
| chr17 | 21639450 | 21639500 |
| chr17 | 21971245 | 21971295 |
| chr17 | 21971093 | 21971143 |
| chr17 | 21973380 | 21973430 |
| chr17 | 21975111 | 21975161 |
| chr17 | 21976921 | 21976971 |
| chr17 | 22047600 | 22047650 |
| chr17 | 22049889 | 22049939 |
| chr17 | 22051925 | 22051975 |
| chr17 | 22053384 | 22053434 |
| chr17 | 22054809 | 22054859 |
| chr17 | 22056570 | 22056620 |
| chr17 | 22337670 | 22337720 |
| chr17 | 25628588 | 25628638 |
| chr17 | 25631116 | 25631166 |

Table 15DX. Repeat Category SVA

| | | |
|---|---|---|
| chr17 | 37864620 | 37864670 |
| chr17 | 37868936 | 37868986 |
| chr10 | 81959991 | 81960041 |
| chr17 | 41685175 | 41685225 |
| chr17 | 42004319 | 42004369 |
| chr17 | 42734324 | 42734374 |
| chr17 | 43076361 | 43076411 |
| chr17 | 45948984 | 45949034 |
| chr17 | 47677535 | 47677585 |
| chr17 | 47701957 | 47702007 |
| chr17 | 47703054 | 47703104 |
| chr10 | 85605701 | 85605751 |
| chr17 | 56397987 | 56398037 |
| chr17 | 56627221 | 56627271 |
| chr17 | 56695603 | 56695653 |
| chr17 | 56761362 | 56761412 |
| chr17 | 59102423 | 59102473 |
| chr17 | 59104693 | 59104743 |
| chr17 | 59528917 | 59528967 |
| chr17 | 60573292 | 60573342 |
| chr17 | 62007246 | 62007296 |
| chr17 | 64030006 | 64030056 |
| chr17 | 64382861 | 64382911 |
| chr17 | 64482980 | 64483030 |
| chr10 | 87878662 | 87878712 |
| chr17 | 76370870 | 76370920 |
| chr17 | 77057926 | 77057976 |
| chr17 | 77059787 | 77059837 |
| chr17 | 78833919 | 78833969 |
| chr17 | 78920815 | 78920865 |
| chr17 | 78921614 | 78921664 |
| chr10 | 92748136 | 92748186 |
| chr17 | 81013243 | 81013293 |
| chr18 | 9420600 | 9420650 |
| chr18 | 10497097 | 10497147 |
| chr10 | 94398851 | 94398901 |
| chr18 | 11598689 | 11598739 |
| chr18 | 11604243 | 11604293 |
| chr18 | 11638686 | 11638736 |
| chr18 | 12233072 | 12233122 |
| chr18 | 12971495 | 12971545 |
| chr18 | 13089283 | 13089333 |
| chr18 | 14002100 | 14002150 |
| chr18 | 14844429 | 14844479 |
| chr18 | 16970446 | 16970496 |
| chr10 | 95394816 | 95394866 |
| chr18 | 28980431 | 28980481 |
| chr18 | 31910823 | 31910873 |
| chr18 | 43605810 | 43605860 |
| chr18 | 44859908 | 44859958 |
| chr10 | 97234931 | 97234981 |
| chr18 | 45357576 | 45357626 |
| chr18 | 45577496 | 45577546 |
| chr18 | 46197026 | 46197076 |
| chr18 | 46387762 | 46387812 |
| chr18 | 50122968 | 50123018 |
| chr18 | 60425731 | 60425781 |
| chr18 | 62943894 | 62943944 |
| chr10 | 98366801 | 98366851 |
| chr18 | 65919396 | 65919446 |
| chr18 | 71840174 | 71840224 |
| chr10 | 98954242 | 98954292 |
| chr10 | 99265275 | 99265325 |
| chr10 | 99576058 | 99576108 |
| chr19 | 3949538 | 3949588 |
| chr10 | 101516416 | 101516466 |
| chr10 | 101700609 | 101700659 |
| chr19 | 8710247 | 8710297 |
| chr19 | 10449737 | 10449787 |
| chr19 | 11274598 | 11274648 |
| chr19 | 11649906 | 11649956 |
| chr19 | 12333644 | 12333694 |
| chr19 | 12438908 | 12438958 |
| chr19 | 12594362 | 12594412 |
| chr19 | 13674463 | 13674513 |
| chr19 | 15334075 | 15334125 |
| chr19 | 17978509 | 17978559 |
| chr10 | 103726614 | 103726664 |
| chr19 | 20036101 | 20036151 |
| chr19 | 20108932 | 20108982 |
| chr19 | 20148629 | 20148679 |
| chr19 | 20506124 | 20506174 |
| chr19 | 21023513 | 21023563 |
| chr19 | 21188097 | 21188147 |
| chr19 | 21234798 | 21234848 |
| chr19 | 21236700 | 21236750 |
| chr19 | 21419107 | 21419157 |
| chr19 | 21461933 | 21461983 |
| chr19 | 21487005 | 21487055 |
| chr19 | 21582097 | 21582147 |
| chr19 | 21589414 | 21589464 |
| chr19 | 21681938 | 21681988 |
| chr19 | 21694870 | 21694920 |
| chr19 | 21841739 | 21841789 |
| chr19 | 22079302 | 22079352 |
| chr19 | 22083508 | 22083558 |
| chr19 | 22296495 | 22296545 |
| chr19 | 22675294 | 22675344 |
| chr19 | 22677851 | 22677901 |
| chr19 | 22900009 | 22900059 |
| chr19 | 23108961 | 23109011 |
| chr19 | 23136141 | 23136191 |
| chr19 | 23147388 | 23147438 |
| chr19 | 23265473 | 23265523 |
| chr19 | 23344675 | 23344725 |
| chr19 | 23394402 | 23394452 |
| chr19 | 23508371 | 23508421 |
| chr19 | 23911260 | 23911310 |
| chr19 | 35131898 | 35131948 |
| chr19 | 39261074 | 39261124 |
| chr19 | 39263427 | 39263477 |
| chr19 | 39314605 | 39314655 |
| chr19 | 40104065 | 40104115 |
| chr19 | 42870509 | 42870559 |
| chr19 | 43305581 | 43305631 |
| chr19 | 43547246 | 43547296 |
| chr19 | 43547461 | 43547511 |
| chr19 | 43981578 | 43981628 |
| chr19 | 43990271 | 43990321 |
| chr10 | 105032521 | 105032571 |
| chr19 | 45307583 | 45307633 |
| chr19 | 45999128 | 45999178 |
| chr19 | 46163266 | 46163316 |
| chr19 | 46165258 | 46165308 |
| chr19 | 46377437 | 46377487 |
| chr19 | 46964552 | 46964602 |
| chr10 | 106209953 | 106210003 |
| chr19 | 47807284 | 47807334 |
| chr19 | 47807539 | 47807589 |
| chr19 | 47836528 | 47836578 |
| chr19 | 47836738 | 47836788 |
| chr19 | 48743784 | 48743834 |
| chr19 | 49684062 | 49684112 |
| chr10 | 112192597 | 112192647 |
| chr19 | 55586783 | 55586833 |
| chr19 | 55777747 | 55777797 |
| chr19 | 57147868 | 57147918 |
| chr19 | 57217319 | 57217369 |
| chr19 | 57870942 | 57870992 |
| chr19 | 57914686 | 57914736 |
| chr19 | 58115312 | 58115362 |
| chr19 | 58417135 | 58417185 |
| chr19 | 58464391 | 58464441 |
| chr19 | 58465975 | 58466025 |
| chr19 | 58782007 | 58782057 |
| chr19 | 59410613 | 59410663 |
| chr10 | 6101851 | 6101901 |
| chr19 | 63609643 | 63609693 |
| chr1 | 6075915 | 6075965 |
| chr1 | 6485917 | 6485967 |
| chr10 | 116211014 | 116211064 |
| chr1 | 7723387 | 7723437 |
| chr1 | 8269871 | 8269921 |
| chr1 | 8681661 | 8681711 |
| chr1 | 8682579 | 8682629 |
| chr1 | 11076259 | 11076309 |
| chr10 | 6253552 | 6253602 |
| chr1 | 20820932 | 20820982 |
| chr1 | 21297955 | 21298005 |
| chr1 | 24478767 | 24478817 |
| chr1 | 24509715 | 24509765 |
| chr1 | 24512635 | 24512685 |
| chr1 | 26528785 | 26528835 |
| chr1 | 26750546 | 26750596 |
| chr1 | 26994783 | 26994833 |
| chr1 | 27786309 | 27786359 |
| chr1 | 30321610 | 30321660 |
| chr10 | 120686024 | 120686074 |
| chr1 | 32941385 | 32941435 |
| chr1 | 32942019 | 32942069 |
| chr1 | 32944060 | 32944110 |
| chr1 | 33325049 | 33325099 |
| chr1 | 34050727 | 34050777 |
| chr1 | 34840779 | 34840829 |
| chr1 | 36972969 | 36973019 |
| chr10 | 121147198 | 121147248 |
| chr1 | 38527309 | 38527359 |
| chr1 | 39114566 | 39114616 |
| chr1 | 39748084 | 39748134 |
| chr1 | 39791216 | 39791266 |
| chr10 | 121640164 | 121640214 |
| chr1 | 44960171 | 44960221 |
| chr1 | 44983957 | 44984007 |
| chr1 | 44995084 | 44995134 |
| chr1 | 45102917 | 45102967 |
| chr1 | 45126460 | 45126510 |
| chr1 | 45376458 | 45376508 |
| chr1 | 45572936 | 45572986 |
| chr1 | 46075784 | 46075834 |
| chr1 | 48220000 | 48220050 |
| chr1 | 48314226 | 48314276 |
| chr1 | 50217707 | 50217757 |
| chr1 | 50662437 | 50662487 |
| chr1 | 50787686 | 50787736 |
| chr1 | 51548093 | 51548143 |
| chr1 | 52706478 | 52706528 |
| chr1 | 54068147 | 54068197 |
| chr10 | 123628332 | 123628382 |
| chr1 | 76001630 | 76001680 |
| chr1 | 77575987 | 77576037 |
| chr1 | 77759401 | 77759451 |
| chr1 | 80923529 | 80923579 |
| chr1 | 86789105 | 86789155 |
| chr1 | 86852333 | 86852383 |
| chr1 | 89730933 | 89730983 |
| chr1 | 91221796 | 91221846 |
| chr1 | 91964013 | 91964063 |
| chr1 | 91964210 | 91964260 |
| chr1 | 96407451 | 96407501 |
| chr1 | 96408015 | 96408065 |
| chr1 | 96700970 | 96701020 |
| chr1 | 98442195 | 98442245 |
| chr1 | 98442407 | 98442457 |
| chr1 | 99007962 | 99008012 |
| chr1 | 99436588 | 99436638 |
| chr1 | 99436839 | 99436889 |
| chr1 | 99786467 | 99786517 |
| chr1 | 106309087 | 106309137 |
| chr1 | 108058362 | 108058412 |
| chr1 | 108426146 | 108426196 |
| chr1 | 108564251 | 108564301 |
| chr1 | 111074871 | 111074921 |
| chr1 | 111371416 | 111371466 |
| chr1 | 111450444 | 111450494 |
| chr1 | 111641250 | 111641300 |
| chr1 | 112481075 | 112481125 |
| chr1 | 112569776 | 112569826 |
| chr1 | 112740854 | 112740904 |
| chr1 | 114084359 | 114084409 |
| chr1 | 114321118 | 114321168 |
| chr1 | 114646723 | 114646773 |
| chr1 | 115202666 | 115202716 |
| chr1 | 117317734 | 117317784 |
| chr1 | 117320217 | 117320267 |
| chr1 | 141723087 | 141723137 |
| chr1 | 143416461 | 143416511 |
| chr1 | 143419906 | 143419956 |
| chr1 | 144039781 | 144039831 |
| chr1 | 146561408 | 146561458 |
| chr10 | 126780633 | 126780683 |
| chr1 | 147918894 | 147918944 |
| chr1 | 148790802 | 148790852 |
| chr1 | 148841397 | 148841447 |
| chr1 | 148958259 | 148958309 |
| chr1 | 149267901 | 149267951 |
| chr1 | 149334791 | 149334841 |
| chr1 | 149385870 | 149385920 |
| chr1 | 149445795 | 149445845 |
| chr1 | 149662123 | 149662173 |
| chr10 | 127233754 | 127233804 |
| chr1 | 151477705 | 151477755 |
| chr1 | 151568711 | 151568761 |
| chr1 | 152887053 | 152887103 |
| chr1 | 153745730 | 153745780 |
| chr1 | 156635625 | 156635675 |
| chr10 | 128266789 | 128266839 |
| chr1 | 157717997 | 157718047 |
| chr1 | 158119199 | 158119249 |
| chr1 | 158216159 | 158216209 |
| chr1 | 158469005 | 158469055 |
| chr1 | 158608730 | 158608780 |
| chr1 | 159608960 | 159609010 |
| chr1 | 159614599 | 159614649 |
| chr1 | 159624064 | 159624114 |
| chr1 | 160655110 | 160655160 |
| chr1 | 160656709 | 160656759 |
| chr1 | 164186577 | 164186627 |
| chr1 | 164928750 | 164928800 |
| chr1 | 164963491 | 164963541 |
| chr1 | 165365335 | 165365385 |
| chr1 | 165993319 | 165993369 |
| chr1 | 167576429 | 167576479 |
| chr1 | 168902433 | 168902483 |
| chr1 | 169780288 | 169780338 |
| chr1 | 171969996 | 171970046 |
| chr1 | 173265792 | 173265842 |
| chr1 | 176249568 | 176249618 |
| chr1 | 179647472 | 179647522 |
| chr1 | 179951552 | 179951602 |
| chr1 | 180635497 | 180635547 |
| chr1 | 183667543 | 183667593 |
| chr1 | 184823767 | 184823817 |
| chr1 | 186397756 | 186397806 |
| chr1 | 194662399 | 194662449 |
| chr1 | 195841881 | 195841931 |
| chr1 | 199448114 | 199448164 |
| chr1 | 199998056 | 199998106 |
| chr1 | 200524238 | 200524288 |
| chr1 | 200987630 | 200987680 |
| chr1 | 206381019 | 206381069 |
| chr1 | 208364987 | 208365037 |
| chr1 | 208450031 | 208450081 |
| chr1 | 208712267 | 208712317 |
| chr1 | 209527408 | 209527458 |
| chr1 | 210008207 | 210008257 |
| chr1 | 210383109 | 210383159 |
| chr1 | 212100487 | 212100537 |
| chr1 | 214754763 | 214754813 |
| chr1 | 217697475 | 217697525 |
| chr1 | 221512399 | 221512449 |
| chr1 | 222777223 | 222777273 |
| chr1 | 222959363 | 222959413 |
| chr1 | 224294417 | 224294467 |
| chr1 | 224874141 | 224874191 |
| chr1 | 228624252 | 228624302 |
| chr1 | 229551038 | 229551088 |
| chr1 | 229552484 | 229552534 |
| chr1 | 231488680 | 231488730 |
| chr1 | 233632164 | 233632214 |
| chr1 | 234332911 | 234332961 |
| chr1 | 234873194 | 234873244 |
| chr1 | 235530678 | 235530728 |
| chr1 | 238620763 | 238620813 |
| chr1 | 242885219 | 242885269 |
| chr1 | 243473979 | 243474029 |
| chr1 | 244141539 | 244141589 |
| chr1 | 244329852 | 244329902 |
| chr1 | 244632046 | 244632096 |
| chr1 | 245774636 | 245774686 |
| chr20 | 3102191 | 3102241 |
| chr20 | 4007499 | 4007549 |
| chr20 | 5428499 | 5428549 |
| chr20 | 5615696 | 5615746 |
| chr20 | 8619097 | 8619147 |
| chr20 | 9361136 | 9361186 |
| chr20 | 11489840 | 11489890 |
| chr20 | 13735794 | 13735844 |
| chr20 | 16204293 | 16204343 |
| chr20 | 19654518 | 19654568 |
| chr10 | 133277383 | 133277433 |
| chr20 | 24600970 | 24601020 |
| chr20 | 25287625 | 25287675 |
| chr20 | 29422434 | 29422484 |
| chr20 | 31453944 | 31453994 |
| chr20 | 31510455 | 31510505 |
| chr20 | 32418550 | 32418600 |
| chr20 | 32489817 | 32489867 |
| chr20 | 32841002 | 32841052 |
| chr20 | 33002555 | 33002605 |
| chr20 | 33005345 | 33005395 |
| chr20 | 36325661 | 36325711 |
| chr20 | 36464063 | 36464113 |
| chr20 | 37694101 | 37694151 |
| chr20 | 37694043 | 37694093 |
| chr20 | 37826042 | 37826092 |
| chr20 | 40870445 | 40870495 |
| chr20 | 42921658 | 42921708 |
| chr20 | 44960382 | 44960432 |
| chr20 | 44961126 | 44961176 |
| chr20 | 49225279 | 49225329 |
| chr20 | 53551561 | 53551611 |
| chr20 | 57674894 | 57674944 |
| chr21 | 15966999 | 15967049 |
| chr21 | 17587571 | 17587621 |
| chr21 | 25989075 | 25989125 |
| chr21 | 28857115 | 28857165 |
| chr21 | 36684328 | 36684378 |
| chr21 | 37959115 | 37959165 |
| chr21 | 46060422 | 46060472 |
| chr10 | 8914307 | 8914357 |
| chr22 | 16899855 | 16899905 |
| chr22 | 17263086 | 17263136 |
| chr22 | 17445438 | 17445488 |
| chr22 | 20356744 | 20356794 |
| chr22 | 21120114 | 21120164 |
| chr22 | 22227124 | 22227174 |
| chr22 | 22786667 | 22786717 |
| chr22 | 23039036 | 23039086 |
| chr22 | 23999369 | 23999419 |
| chr22 | 27275106 | 27275156 |
| chr10 | 9320215 | 9320265 |
| chr22 | 28272240 | 28272290 |
| chr22 | 28364660 | 28364710 |
| chr22 | 28780217 | 28780267 |
| chr22 | 29746663 | 29746713 |
| chr22 | 29748744 | 29748794 |
| chr22 | 29905657 | 29905707 |
| chr22 | 33404992 | 33405042 |
| chr22 | 34959787 | 34959837 |
| chr22 | 37620291 | 37620341 |
| chr22 | 37620133 | 37620183 |
| chr22 | 37650110 | 37650160 |
| chr10 | 9901903 | 9901953 |
| chr22 | 39932992 | 39933042 |
| chr22 | 40271217 | 40271267 |
| chr22 | 41599274 | 41599324 |
| chr11 | 3383795 | 3383845 |
| chr22 | 48029596 | 48029646 |
| chr22 | 48610304 | 48610354 |
| chrX | 6987354 | 6987404 |
| chrX | 6989970 | 6990020 |
| chrX | 7881727 | 7881777 |
| chr11 | 4123633 | 4123683 |
| chrX | 12992230 | 12992280 |
| chr11 | 4222421 | 4222471 |
| chrX | 16345668 | 16345718 |
| chrX | 16349210 | 16349260 |
| chrX | 16625550 | 16625600 |
| chrX | 22902646 | 22902696 |
| chrX | 24089807 | 24089857 |
| chrX | 25284717 | 25284767 |
| chrX | 26838354 | 26838404 |
| chr11 | 5920275 | 5920325 |
| chr11 | 5945739 | 5945789 |
| chrX | 28714872 | 28714922 |
| chrX | 35640551 | 35640601 |
| chrX | 36598374 | 36598424 |
| chrX | 39753937 | 39753987 |
| chrX | 40275031 | 40275081 |
| chrX | 41175076 | 41175126 |
| chrX | 41365260 | 41365310 |
| chrX | 43815528 | 43815578 |
| chrX | 43947517 | 43947567 |
| chrX | 44410035 | 44410085 |
| chrX | 45302502 | 45302552 |
| chrX | 47019969 | 47020019 |
| chrX | 49779322 | 49779372 |
| chrX | 51823646 | 51823696 |
| chrX | 52714800 | 52714850 |
| chrX | 53208387 | 53208437 |
| chrX | 53729273 | 53729323 |
| chrX | 54365148 | 54365198 |
| chrX | 54741759 | 54741809 |
| chrX | 55373737 | 55373787 |
| chrX | 61277107 | 61277157 |
| chrX | 61913176 | 61913226 |
| chrX | 64062729 | 64062779 |
| chrX | 66412501 | 66412551 |
| chrX | 66565428 | 66565478 |
| chrX | 67508653 | 67508703 |
| chr11 | 8508575 | 8508625 |
| chrX | 68209070 | 68209120 |
| chrX | 68436975 | 68437025 |
| chrX | 68459317 | 68459367 |
| chrX | 68767264 | 68767314 |
| chrX | 69387244 | 69387294 |
| chrX | 69386927 | 69386977 |
| chrX | 69732487 | 69732537 |
| chrX | 69762844 | 69762894 |
| chrX | 70266731 | 70266781 |
| chrX | 70769609 | 70769659 |
| chrX | 71107436 | 71107486 |
| chrX | 71109494 | 71109544 |
| chrX | 71153900 | 71153950 |
| chrX | 71968944 | 71968994 |
| chrX | 72254796 | 72254846 |
| chrX | 72499987 | 72500037 |
| chrX | 73420418 | 73420468 |
| chrX | 73422875 | 73422925 |
| chrX | 75153113 | 75153163 |
| chrX | 75367287 | 75367337 |
| chr11 | 8963146 | 8963196 |
| chrX | 75944307 | 75944357 |
| chrX | 78409296 | 78409346 |
| chrX | 78723449 | 78723499 |
| chrX | 85060151 | 85060201 |
| chrX | 85492630 | 85492680 |
| chrX | 94078171 | 94078221 |
| chrX | 98749981 | 98750031 |
| chrX | 99000336 | 99000386 |
| chrX | 99766108 | 99766158 |
| chrX | 120487623 | 120487673 |
| chrX | 121419432 | 121419482 |
| chrX | 121565043 | 121565093 |
| chrX | 121960814 | 121960864 |
| chrX | 124480096 | 124480146 |
| chrX | 124514656 | 124514706 |
| chrX | 129166861 | 129166911 |
| chrX | 132126485 | 132126535 |
| chrX | 132799266 | 132799316 |
| chrX | 134789452 | 134789502 |
| chrX | 136071210 | 136071260 |
| chrX | 143798909 | 143798959 |
| chrX | 145741311 | 145741361 |
| chrX | 149386401 | 149386451 |
| chrX | 152469870 | 152469920 |
| chrX | 152875190 | 152875240 |
| chrY | 3866599 | 3866649 |
| chrY | 8489041 | 8489091 |
| chrY | 13574876 | 13574926 |
| chrY | 13999269 | 13999319 |
| chrY | 20592134 | 20592184 |
| chrY | 21559578 | 21559628 |
| chr2 | 117882 | 117932 |
| chr11 | 15672337 | 15672387 |
| chr2 | 3204573 | 3204623 |
| chr2 | 4979181 | 4979231 |
| chr2 | 7499488 | 7499538 |
| chr11 | 17037506 | 17037556 |
| chr2 | 11066028 | 11066078 |
| chr2 | 15382130 | 15382180 |
| chr2 | 15775102 | 15775152 |
| chr2 | 15841628 | 15841678 |
| chr11 | 18108009 | 18108059 |
| chr2 | 24598129 | 24598179 |
| chr2 | 25732866 | 25732916 |
| chr2 | 25734107 | 25734157 |
| chr2 | 25830525 | 25830575 |
| chr2 | 25919797 | 25919847 |
| chr2 | 26569299 | 26569349 |
| chr2 | 26863096 | 26863146 |
| chr2 | 27738824 | 27738874 |
| chr2 | 27756436 | 27756486 |
| chr2 | 27792529 | 27792579 |
| chr2 | 32648272 | 32648322 |
| chr2 | 32955231 | 32955281 |
| chr2 | 33961641 | 33961691 |
| chr2 | 34333508 | 34333558 |
| chr2 | 38135630 | 38135680 |
| chr2 | 38165915 | 38165965 |
| chr2 | 38179276 | 38179326 |
| chr11 | 19847158 | 19847208 |
| chr2 | 39830910 | 39830960 |
| chr2 | 41947661 | 41947711 |
| chr2 | 41950404 | 41950454 |
| chr2 | 42196472 | 42196522 |
| chr2 | 42217298 | 42217348 |
| chr2 | 42808785 | 42808835 |
| chr2 | 43265845 | 43265895 |
| chr2 | 43498271 | 43498321 |
| chr2 | 43500315 | 43500365 |
| chr2 | 43880430 | 43880480 |
| chr2 | 44263336 | 44263386 |
| chr2 | 44791739 | 44791789 |
| chr11 | 20849978 | 20850028 |
| chr2 | 47636478 | 47636528 |
| chr2 | 48756212 | 48756262 |
| chr2 | 48815338 | 48815388 |
| chr2 | 50614095 | 50614145 |
| chr2 | 53525354 | 53525404 |
| chr2 | 55907293 | 55907343 |
| chr2 | 55976416 | 55976466 |
| chr2 | 62832413 | 62832463 |
| chr2 | 63925042 | 63925092 |
| chr2 | 64011757 | 64011807 |
| chr2 | 64242968 | 64243018 |
| chr2 | 64713834 | 64713884 |
| chr2 | 65803506 | 65803556 |
| chr2 | 74263993 | 74264043 |
| chr2 | 74325177 | 74325227 |
| chr2 | 75927671 | 75927721 |
| chr2 | 86548260 | 86548310 |
| chr2 | 86748035 | 86748085 |
| chr2 | 86751223 | 86751273 |
| chr11 | 28415051 | 28415101 |
| chr2 | 89128081 | 89128131 |
| chr2 | 95393385 | 95393435 |
| chr2 | 98340411 | 98340461 |
| chr2 | 105488366 | 105488416 |
| chr2 | 108965316 | 108965366 |
| chr2 | 110558671 | 110558721 |
| chr2 | 113949003 | 113949053 |
| chr2 | 120698151 | 120698201 |
| chr2 | 126130693 | 126130743 |
| chr2 | 127971499 | 127971549 |
| chr2 | 128203487 | 128203537 |
| chr2 | 129058858 | 129058908 |
| chr2 | 129270462 | 129270512 |
| chr2 | 130659025 | 130659075 |
| chr2 | 130683880 | 130683930 |
| chr2 | 135032003 | 135032053 |
| chr2 | 135936087 | 135936137 |
| chr2 | 136243700 | 136243750 |
| chr2 | 136675380 | 136675430 |
| chr11 | 33971889 | 33971939 |
| chr2 | 144959683 | 144959733 |
| chr2 | 152935240 | 152935290 |
| chr2 | 153218611 | 153218661 |
| chr2 | 157351779 | 157351829 |
| chr2 | 157511773 | 157511823 |
| chr2 | 159370579 | 159370629 |
| chr2 | 161499322 | 161499372 |
| chr2 | 163528626 | 163528676 |
| chr2 | 170385154 | 170385204 |
| chr2 | 170387761 | 170387811 |
| chr2 | 170563960 | 170564010 |
| chr2 | 170565362 | 170565412 |
| chr2 | 170635216 | 170635266 |
| chr2 | 171208056 | 171208106 |
| chr2 | 172834746 | 172834796 |
| chr2 | 172833072 | 172833122 |
| chr2 | 172960183 | 172960233 |
| chr2 | 173604336 | 173604386 |
| chr2 | 173767165 | 173767215 |
| chr2 | 176174649 | 176174699 |
| chr2 | 178913322 | 178913372 |
| chr11 | 37418083 | 37418133 |
| chr2 | 183453258 | 183453308 |
| chr2 | 187222385 | 187222435 |
| chr2 | 188705461 | 188705511 |
| chr2 | 190720813 | 190720863 |
| chr2 | 191499257 | 191499307 |
| chr2 | 192466848 | 192466898 |
| chr2 | 192808333 | 192808383 |
| chr2 | 193960514 | 193960564 |
| chr2 | 198764906 | 198764956 |
| chr2 | 198989211 | 198989261 |
| chr2 | 198991529 | 198991579 |
| chr2 | 201752061 | 201752111 |
| chr2 | 202109174 | 202109224 |
| chr2 | 202233824 | 202233874 |
| chr2 | 202349139 | 202349189 |
| chr2 | 202630818 | 202630868 |
| chr2 | 203601228 | 203601278 |
| chr2 | 203783341 | 203783391 |
| chr2 | 204439048 | 204439098 |
| chr11 | 41288808 | 41288858 |
| chr2 | 207425287 | 207425337 |
| chr2 | 207467114 | 207467164 |
| chr2 | 209445754 | 209445804 |
| chr2 | 212017257 | 212017307 |
| chr2 | 217295015 | 217295065 |
| chr2 | 220819339 | 220819389 |
| chr2 | 222958714 | 222958764 |
| chr2 | 223884739 | 223884789 |
| chr2 | 230809338 | 230809388 |
| chr2 | 233974469 | 233974519 |
| chr2 | 234920756 | 234920806 |
| chr10 | 12273291 | 12273341 |
| chr2 | 240172343 | 240172393 |
| chr2 | 240582472 | 240582522 |
| chr2 | 243320065 | 243320115 |
| chr3 | 2933957 | 2934007 |
| chr3 | 5130570 | 5130620 |
| chr3 | 5260197 | 5260247 |
| chr3 | 5300631 | 5300681 |
| chr3 | 8342595 | 8342645 |
| chr3 | 8457932 | 8457982 |
| chr3 | 9575852 | 9575902 |
| chr3 | 9587518 | 9587568 |
| chr11 | 46884329 | 46884379 |
| chr3 | 10087134 | 10087184 |
| chr3 | 10433363 | 10433413 |
| chr11 | 47082844 | 47082894 |
| chr3 | 11504319 | 11504369 |
| chr3 | 12865423 | 12865473 |
| chr3 | 15077438 | 15077488 |
| chr3 | 15178450 | 15178500 |
| chr3 | 15758154 | 15758204 |
| chr3 | 16013215 | 16013265 |
| chr3 | 16997646 | 16997696 |
| chr3 | 20565198 | 20565248 |
| chr3 | 23889024 | 23889074 |
| chr3 | 32182170 | 32182220 |
| chr3 | 38054848 | 38054898 |
| chr3 | 38067523 | 38067573 |
| chr11 | 47877944 | 47877994 |
| chr3 | 39229007 | 39229057 |
| chr3 | 39657831 | 39657881 |
| chr3 | 41307227 | 41307277 |
| chr3 | 41843453 | 41843503 |
| chr3 | 41985386 | 41985436 |
| chr3 | 42798928 | 42798978 |
| chr3 | 42912502 | 42912552 |
| chr11 | 48942104 | 48942154 |
| chr3 | 44824130 | 44824180 |
| chr3 | 47259349 | 47259399 |
| chr3 | 47311155 | 47311205 |
| chr11 | 50048250 | 50048300 |
| chr3 | 50082426 | 50082476 |
| chr11 | 55687513 | 55687563 |
| chr3 | 50800243 | 50800293 |
| chr3 | 51244702 | 51244752 |
| chr3 | 51304612 | 51304662 |
| chr3 | 51601378 | 51601428 |
| chr11 | 56039982 | 56040032 |
| chr3 | 56576597 | 56576647 |
| chr3 | 57330787 | 57330837 |
| chr3 | 57689952 | 57690002 |
| chr3 | 58233798 | 58233848 |
| chr3 | 58834519 | 58834569 |
| chr10 | 12566958 | 12567008 |
| chr3 | 60870782 | 60870832 |
| chr3 | 61336734 | 61336784 |
| chr10 | 12569773 | 12569823 |
| chr3 | 61378741 | 61378791 |
| chr3 | 61613918 | 61613968 |
| chr11 | 57260710 | 57260760 |
| chr3 | 67426687 | 67426737 |
| chr11 | 57487558 | 57487608 |
| chr3 | 71898403 | 71898453 |
| chr3 | 72058973 | 72059023 |
| chr3 | 77134394 | 77134444 |
| chr3 | 82553872 | 82553922 |
| chr3 | 84888267 | 84888317 |
| chr3 | 95084603 | 95084653 |
| chr3 | 96783608 | 96783658 |
| chr11 | 59127738 | 59127788 |
| chr11 | 59243434 | 59243484 |
| chr3 | 101519040 | 101519090 |
| chr3 | 102478471 | 102478521 |
| chr3 | 102591681 | 102591731 |
| chr3 | 104295845 | 104295895 |
| chr3 | 104571369 | 104571419 |
| chr3 | 107115684 | 107115734 |
| chr3 | 107151613 | 107151663 |
| chr11 | 59744942 | 59744992 |
| chr3 | 109065331 | 109065381 |
| chr3 | 110164908 | 110164958 |
| chr11 | 59993343 | 59993393 |
| chr11 | 60033643 | 60033693 |
| chr11 | 60052632 | 60052682 |
| chr3 | 114087325 | 114087375 |
| chr3 | 114497959 | 114498009 |
| chr3 | 114888903 | 114888953 |
| chr3 | 117558333 | 117558383 |
| chr3 | 118091316 | 118091366 |
| chr3 | 119759055 | 119759105 |
| chr3 | 120282325 | 120282375 |
| chr3 | 121454943 | 121454993 |
| chr3 | 123203406 | 123203456 |
| chr3 | 123242235 | 123242285 |
| chr3 | 123821259 | 123821309 |
| chr3 | 125791162 | 125791212 |
| chr11 | 60710165 | 60710215 |
| chr3 | 128333637 | 128333687 |
| chr3 | 128349536 | 128349586 |
| chr3 | 129812448 | 129812498 |
| chr3 | 130380868 | 130380918 |
| chr3 | 130404254 | 130404304 |
| chr3 | 131906916 | 131906966 |
| chr3 | 132991237 | 132991287 |
| chr3 | 133511983 | 133512033 |
| chr3 | 137764369 | 137764419 |
| chr3 | 138239288 | 138239338 |
| chr3 | 138911732 | 138911782 |
| chr3 | 139561450 | 139561500 |
| chr3 | 139825401 | 139825451 |
| chr3 | 142070326 | 142070376 |
| chr3 | 144113304 | 144113354 |
| chr3 | 144127650 | 144127700 |
| chr3 | 146521762 | 146521812 |
| chr3 | 150420105 | 150420155 |
| chr3 | 150483743 | 150483793 |
| chr3 | 157001239 | 157001289 |
| chr3 | 158218436 | 158218486 |
| chr3 | 161218343 | 161218393 |
| chr3 | 165822502 | 165822552 |
| chr3 | 176126000 | 176126050 |
| chr3 | 176171693 | 176171743 |
| chr3 | 178452418 | 178452468 |
| chr3 | 178931306 | 178931356 |
| chr3 | 178952919 | 178952969 |
| chr3 | 180070735 | 180070785 |
| chr3 | 180330420 | 180330470 |
| chr3 | 180767784 | 180767834 |
| chr3 | 182965395 | 182965445 |
| chr3 | 184645997 | 184646047 |
| chr3 | 187851359 | 187851409 |
| chr3 | 187911898 | 187911948 |
| chr3 | 187924312 | 187924362 |
| chr3 | 191512025 | 191512075 |
| chr3 | 192163079 | 192163129 |
| chr3 | 195440429 | 195440479 |
| chr4 | 305760 | 305810 |
| chr4 | 378714 | 378764 |
| chr4 | 535300 | 535350 |
| chr4 | 575899 | 575949 |
| chr4 | 1790346 | 1790396 |
| chr4 | 3127986 | 3128036 |
| chr4 | 6903951 | 6904001 |
| chr11 | 64244542 | 64244592 |
| chr4 | 13514780 | 13514830 |
| chr4 | 16146889 | 16146939 |
| chr4 | 25002162 | 25002212 |
| chr4 | 28268326 | 28268376 |
| chr4 | 37566942 | 37566992 |
| chr4 | 38705577 | 38705627 |
| chr4 | 38707069 | 38707119 |
| chr4 | 38705526 | 38705576 |
| chr4 | 40040961 | 40041011 |
| chr4 | 40047464 | 40047514 |
| chr4 | 41408307 | 41408357 |
| chr4 | 41409959 | 41410009 |
| chr4 | 45124453 | 45124503 |
| chr4 | 45126888 | 45126938 |
| chr4 | 48104192 | 48104242 |
| chr4 | 48210433 | 48210483 |
| chr4 | 48282449 | 48282499 |
| chr4 | 53774448 | 53774498 |
| chr4 | 54021168 | 54021218 |
| chr4 | 54038863 | 54038913 |
| chr4 | 54813739 | 54813789 |
| chr4 | 54974765 | 54974815 |
| chr4 | 55108506 | 55108556 |
| chr4 | 57476050 | 57476100 |
| chr4 | 57954046 | 57954096 |
| chr4 | 58007002 | 58007052 |
| chr11 | 65305886 | 65305936 |
| chr4 | 64787918 | 64787968 |
| chr4 | 65769243 | 65769293 |
| chr4 | 68249466 | 68249516 |
| chr4 | 70942426 | 70942476 |
| chr4 | 70944355 | 70944405 |
| chr4 | 70942485 | 70942535 |
| chr4 | 71218992 | 71219042 |
| chr4 | 79364394 | 79364444 |
| chr4 | 79387535 | 79387585 |
| chr4 | 79926832 | 79926882 |
| chr4 | 80625236 | 80625286 |
| chr4 | 83456560 | 83456610 |
| chr4 | 84566567 | 84566617 |
| chr4 | 85324228 | 85324278 |
| chr4 | 92548149 | 92548199 |
| chr4 | 95536618 | 95536668 |
| chr4 | 99710973 | 99711023 |
| chr4 | 106490235 | 106490285 |
| chr4 | 106881271 | 106881321 |
| chr4 | 107701499 | 107701549 |
| chr4 | 110983061 | 110983111 |
| chr4 | 111057630 | 111057680 |
| chr4 | 111059573 | 111059623 |
| chr4 | 115175721 | 115175771 |
| chr4 | 120173124 | 120173174 |
| chr4 | 122617937 | 122617987 |
| chr4 | 140238915 | 140238965 |
| chr4 | 147192912 | 147192962 |
| chr4 | 148857810 | 148857860 |
| chr4 | 152159877 | 152159927 |
| chr4 | 152452363 | 152452413 |
| chr4 | 152463170 | 152463220 |
| chr4 | 152666410 | 152666460 |
| chr4 | 152714116 | 152714166 |
| chr4 | 152754814 | 152754864 |
| chr4 | 153024266 | 153024316 |
| chr4 | 154264872 | 154264922 |
| chr4 | 154366316 | 154366366 |
| chr11 | 67021005 | 67021055 |
| chr4 | 159135764 | 159135814 |
| chr4 | 159136799 | 159136849 |
| chr4 | 160244262 | 160244312 |
| chr4 | 160257882 | 160257932 |
| chr4 | 160380069 | 160380119 |
| chr4 | 160381966 | 160382016 |
| chr11 | 67141506 | 67141556 |
| chr4 | 161439611 | 161439661 |
| chr4 | 166246269 | 166246319 |
| chr4 | 166520004 | 166520054 |
| chr4 | 171186399 | 171186449 |
| chr4 | 171296972 | 171297022 |
| chr4 | 171446308 | 171446358 |
| chr4 | 171784156 | 171784206 |
| chr4 | 174800413 | 174800463 |
| chr4 | 175912782 | 175912832 |
| chr4 | 176203954 | 176204004 |
| chr4 | 176308166 | 176308216 |
| chr4 | 182562933 | 182562983 |
| chr4 | 184658760 | 184658810 |
| chr4 | 185354845 | 185354895 |
| chr4 | 185436887 | 185436937 |
| chr11 | 68019296 | 68019346 |
| chr4 | 186858654 | 186858704 |
| chr4 | 189274453 | 189274503 |
| chr4 | 189684997 | 189685047 |
| chr5 | 1638927 | 1638977 |
| chr5 | 16330731 | 16330781 |
| chr5 | 32085228 | 32085278 |
| chr5 | 34353384 | 34353434 |
| chr11 | 69869284 | 69869334 |
| chr5 | 37059846 | 37059896 |
| chr5 | 37565317 | 37565367 |
| chr5 | 37610192 | 37610242 |
| chr5 | 39319088 | 39319138 |
| chr5 | 39379033 | 39379083 |
| chr5 | 40845960 | 40846010 |
| chr11 | 70029414 | 70029464 |
| chr5 | 43024757 | 43024807 |
| chr5 | 43134762 | 43134812 |
| chr5 | 43556883 | 43556933 |
| chr5 | 43567004 | 43567054 |
| chr5 | 43624754 | 43624804 |
| chr5 | 43712487 | 43712537 |
| chr5 | 43792676 | 43792726 |
| chr5 | 43869392 | 43869442 |
| chr5 | 45936559 | 45936609 |
| chr5 | 49919632 | 49919682 |
| chr5 | 50087211 | 50087261 |
| chr5 | 53692253 | 53692303 |
| chr5 | 53733108 | 53733158 |
| chr5 | 55351451 | 55351501 |
| chr5 | 55570573 | 55570623 |
| chr5 | 55710450 | 55710500 |
| chr5 | 56830192 | 56830242 |
| chr5 | 57876806 | 57876856 |
| chr5 | 59743627 | 59743677 |
| chr5 | 61826334 | 61826384 |
| chr5 | 62394801 | 62394851 |
| chr5 | 63537275 | 63537325 |
| chr5 | 65270541 | 65270591 |
| chr5 | 65289682 | 65289732 |
| chr5 | 67636126 | 67636176 |
| chr5 | 70906132 | 70906182 |
| chr5 | 70932735 | 70932785 |
| chr11 | 70606443 | 70606493 |
| chr5 | 74652201 | 74652251 |
| chr5 | 75716644 | 75716694 |
| chr5 | 75931791 | 75931841 |
| chr5 | 76365845 | 76365895 |
| chr5 | 76424330 | 76424380 |
| chr5 | 77140242 | 77140292 |
| chr5 | 77174987 | 77175037 |
| chr5 | 77771976 | 77772026 |
| chr5 | 80204338 | 80204388 |
| chr5 | 80323798 | 80323848 |
| chr5 | 81518850 | 81518900 |
| chr5 | 81521351 | 81521401 |
| chr5 | 82817681 | 82817731 |
| chr5 | 83692597 | 83692647 |
| chr5 | 87994980 | 87995030 |
| chr5 | 93396334 | 93396384 |
| chr5 | 100198261 | 100198311 |
| chr5 | 105270759 | 105270809 |
| chr5 | 107972342 | 107972392 |
| chr5 | 108755363 | 108755413 |
| chr5 | 109245612 | 109245662 |
| chr5 | 111772160 | 111772210 |
| chr5 | 112367724 | 112367774 |
| chr5 | 117012722 | 117012772 |
| chr5 | 117693901 | 117693951 |
| chr5 | 118376343 | 118376393 |
| chr11 | 71940955 | 71941005 |
| chr5 | 118534935 | 118534985 |
| chr11 | 72346401 | 72346451 |
| chr5 | 133269558 | 133269608 |
| chr5 | 134294759 | 134294809 |
| chr5 | 134821989 | 134822039 |
| chr5 | 137098703 | 137098753 |
| chr5 | 138188367 | 138188417 |
| chr5 | 138223846 | 138223896 |
| chr5 | 138468282 | 138468332 |
| chr5 | 138525334 | 138525384 |
| chr5 | 138637318 | 138637368 |
| chr5 | 139836744 | 139836794 |
| chr5 | 141485397 | 141485447 |
| chr11 | 73335602 | 73335652 |
| chr11 | 73375081 | 73375131 |
| chr11 | 73567595 | 73567645 |
| chr11 | 73765747 | 73765797 |
| chr5 | 149079492 | 149079542 |
| chr5 | 151122935 | 151122985 |
| chr5 | 151888965 | 151889015 |
| chr5 | 152445451 | 152445501 |
| chr5 | 152706035 | 152706085 |
| chr5 | 154733518 | 154733568 |
| chr5 | 156347832 | 156347882 |
| chr5 | 157445895 | 157445945 |
| chr5 | 162992490 | 162992540 |
| chr5 | 165327207 | 165327257 |
| chr5 | 167266580 | 167266630 |
| chr5 | 167269126 | 167269176 |
| chr5 | 170555625 | 170555675 |
| chr5 | 170812871 | 170812921 |
| chr11 | 75316270 | 75316320 |
| chr5 | 175660174 | 175660224 |
| chr5 | 176368289 | 176368339 |
| chr11 | 76371315 | 76371365 |
| chr5 | 179179616 | 179179666 |
| chr11 | 76534761 | 76534811 |
| chr5 | 180308543 | 180308593 |
| chr6 | 4982114 | 4982164 |
| chr6 | 5781917 | 5781967 |
| chr6 | 13241543 | 13241593 |
| chr6 | 15296543 | 15296593 |
| chr6 | 15525144 | 15525194 |
| chr6 | 15835798 | 15835848 |
| chr11 | 78316902 | 78316952 |
| chr11 | 78629649 | 78629699 |
| chr6 | 17842796 | 17842846 |
| chr11 | 78936415 | 78936465 |
| chr11 | 79241342 | 79241392 |
| chr6 | 20046727 | 20046777 |
| chr6 | 21906600 | 21906650 |
| chr6 | 21960749 | 21960799 |
| chr6 | 22943720 | 22943770 |
| chr6 | 24522959 | 24523009 |
| chr6 | 25695119 | 25695169 |
| chr11 | 81618370 | 81618420 |
| chr6 | 27562392 | 27562442 |
| chr6 | 27605851 | 27605901 |
| chr6 | 27904250 | 27904300 |
| chr6 | 28578886 | 28578936 |
| chr11 | 82569779 | 82569829 |
| chr6 | 29793396 | 29793446 |
| chr6 | 29969993 | 29970043 |
| chr6 | 30007332 | 30007382 |
| chr6 | 30296612 | 30296662 |
| chr11 | 82755217 | 82755267 |
| chr6 | 31068038 | 31068088 |
| chr6 | 31142316 | 31142366 |
| chr6 | 31927680 | 31927730 |
| chr6 | 32047682 | 32047732 |
| chr6 | 32425122 | 32425172 |
| chr6 | 33075498 | 33075548 |
| chr6 | 33372672 | 33372722 |
| chr6 | 33846472 | 33846522 |
| chr6 | 34411459 | 34411509 |
| chr6 | 34800143 | 34800193 |
| chr6 | 35354218 | 35354268 |
| chr6 | 35355721 | 35355771 |
| chr11 | 85546999 | 85547049 |
| chr6 | 38979060 | 38979110 |
| chr6 | 40830583 | 40830633 |
| chr6 | 41630683 | 41630733 |
| chr6 | 42481945 | 42481995 |
| chr11 | 86208950 | 86209000 |
| chr6 | 43439030 | 43439080 |
| chr6 | 44331347 | 44331397 |
| chr6 | 47459498 | 47459548 |
| chr6 | 47521404 | 47521454 |
| chr6 | 50825357 | 50825407 |
| chr6 | 51892806 | 51892856 |
| chr6 | 53218016 | 53218066 |
| chr6 | 55822642 | 55822692 |
| chr6 | 56735593 | 56735643 |
| chr6 | 57222629 | 57222679 |
| chr6 | 57959000 | 57959050 |
| chr6 | 58563155 | 58563205 |
| chr6 | 58630865 | 58630915 |
| chr6 | 63870774 | 63870824 |
| chr6 | 69827387 | 69827437 |
| chr6 | 70137007 | 70137057 |
| chr6 | 70139399 | 70139449 |
| chr6 | 70671601 | 70671651 |
| chr6 | 71971793 | 71971843 |
| chr6 | 74306245 | 74306295 |
| chr6 | 74557303 | 74557353 |
| chr6 | 74620521 | 74620571 |
| chr6 | 74631362 | 74631412 |
| chr6 | 74960358 | 74960408 |
| chr11 | 90485942 | 90485992 |
| chr6 | 76148174 | 76148224 |
| chr6 | 84949859 | 84949909 |
| chr6 | 87943856 | 87943906 |
| chr6 | 88440400 | 88440450 |
| chr6 | 88442417 | 88442467 |
| chr6 | 88457425 | 88457475 |
| chr6 | 100144530 | 100144580 |
| chr11 | 93369556 | 93369606 |
| chr6 | 105483836 | 105483886 |
| chr6 | 108169879 | 108169929 |
| chr6 | 108301147 | 108301197 |
| chr6 | 108320119 | 108320169 |
| chr6 | 109412841 | 109412891 |
| chr6 | 109414914 | 109414964 |
| chr6 | 111384995 | 111385045 |
| chr6 | 112415165 | 112415215 |
| chr6 | 112875966 | 112876016 |
| chr6 | 112895991 | 112896041 |
| chr6 | 114298275 | 114298325 |
| chr11 | 94743675 | 94743725 |
| chr11 | 94903240 | 94903290 |
| chr6 | 122031226 | 122031276 |
| chr6 | 123152233 | 123152283 |
| chr6 | 126071661 | 126071711 |
| chr6 | 129389416 | 129389466 |
| chr6 | 131103305 | 131103355 |
| chr6 | 131446015 | 131446065 |
| chr6 | 131445396 | 131445446 |
| chr6 | 135666863 | 135666913 |
| chr11 | 98179198 | 98179248 |
| chr6 | 144736050 | 144736100 |
| chr6 | 144772891 | 144772941 |
| chr6 | 148481686 | 148481736 |
| chr6 | 150138187 | 150138237 |
| chr6 | 151801723 | 151801773 |
| chr6 | 154616129 | 154616179 |
| chr6 | 155318861 | 155318911 |
| chr6 | 158458359 | 158458409 |
| chr6 | 158678351 | 158678401 |
| chr6 | 159737485 | 159737535 |
| chr6 | 159738545 | 159738595 |
| chr6 | 162668401 | 162668451 |
| chr11 | 105725765 | 105725815 |
| chr6 | 170013381 | 170013431 |
| chr11 | 107320113 | 107320163 |
| chr7 | 1945044 | 1945094 |
| chr7 | 2053522 | 2053572 |
| chr7 | 2086044 | 2086094 |
| chr7 | 2319330 | 2319380 |
| chr11 | 108553805 | 108553855 |
| chr7 | 6007030 | 6007080 |
| chr7 | 6759932 | 6759982 |
| chr7 | 8675270 | 8675320 |
| chr7 | 16575222 | 16575272 |
| chr7 | 20451686 | 20451736 |
| chr7 | 22828634 | 22828684 |
| chr7 | 24799709 | 24799759 |
| chr7 | 25929316 | 25929366 |
| chr7 | 25959456 | 25959506 |
| chr7 | 26043031 | 26043081 |
| chr7 | 28621555 | 28621605 |
| chr7 | 29045465 | 29045515 |
| chr7 | 29742896 | 29742946 |
| chr11 | 111614658 | 111614708 |
| chr7 | 30935952 | 30936002 |
| chr7 | 32169759 | 32169809 |
| chr7 | 32536766 | 32536816 |
| chr7 | 33085329 | 33085379 |
| chr7 | 33086237 | 33086287 |
| chr7 | 35977068 | 35977118 |
| chr7 | 37074698 | 37074748 |
| chr11 | 112931818 | 112931868 |
| chr7 | 44652531 | 44652581 |
| chr7 | 44773074 | 44773124 |
| chr7 | 47701973 | 47702023 |
| chr7 | 47738995 | 47739045 |
| chr7 | 50268181 | 50268231 |
| chr7 | 51018528 | 51018578 |
| chr7 | 54252876 | 54252926 |
| chr7 | 55030827 | 55030877 |
| chr7 | 55959316 | 55959366 |
| chr7 | 62746739 | 62746789 |
| chr7 | 63611692 | 63611742 |
| chr7 | 63682520 | 63682570 |
| chr7 | 63841399 | 63841449 |
| chr7 | 65491294 | 65491344 |
| chr7 | 66959479 | 66959529 |
| chr11 | 115754724 | 115754774 |
| chr7 | 75195014 | 75195064 |
| chr7 | 82365882 | 82365932 |
| chr7 | 87508754 | 87508804 |
| chr7 | 89394175 | 89394225 |
| chr7 | 89986298 | 89986348 |
| chr7 | 91129839 | 91129889 |
| chr7 | 91267518 | 91267568 |
| chr7 | 94090765 | 94090815 |
| chr7 | 96401185 | 96401235 |
| chr7 | 99733707 | 99733757 |
| chr7 | 99949080 | 99949130 |
| chr7 | 99951893 | 99951943 |
| chr7 | 102270270 | 102270320 |
| chr7 | 107050030 | 107050080 |
| chr7 | 107456084 | 107456134 |
| chr7 | 110809782 | 110809832 |
| chr7 | 111751609 | 111751659 |
| chr7 | 115832583 | 115832633 |
| chr7 | 127656826 | 127656876 |
| chr7 | 127771776 | 127771826 |
| chr7 | 127882691 | 127882741 |
| chr7 | 128410119 | 128410169 |
| chr7 | 128668004 | 128668054 |
| chr7 | 128856947 | 128856997 |
| chr7 | 130856943 | 130856993 |
| chr7 | 132084922 | 132084972 |
| chr7 | 132277862 | 132277912 |
| chr11 | 118400170 | 118400220 |
| chr7 | 135314001 | 135314051 |
| chr7 | 137478490 | 137478540 |
| chr7 | 138323984 | 138324034 |
| chr7 | 138325521 | 138325571 |
| chr7 | 138529099 | 138529149 |
| chr7 | 139221997 | 139222047 |
| chr7 | 141724082 | 141724132 |
| chr7 | 142231577 | 142231627 |
| chr7 | 142231257 | 142231307 |
| chr7 | 146891648 | 146891698 |
| chr11 | 118917246 | 118917296 |
| chr7 | 148369249 | 148369299 |
| chr7 | 148422005 | 148422055 |
| chr7 | 148642197 | 148642247 |
| chr7 | 149594275 | 149594325 |
| chr7 | 151676285 | 151676335 |
| chr10 | 17676024 | 17676074 |
| chr7 | 154218861 | 154218911 |
| chr7 | 154279852 | 154279902 |
| chr8 | 90802 | 90852 |
| chr8 | 913745 | 913795 |
| chr8 | 5569478 | 5569528 |
| chr8 | 9049806 | 9049856 |
| chr8 | 9260307 | 9260357 |
| chr8 | 9260370 | 9260420 |
| chr8 | 15314670 | 15314720 |
| chr8 | 21579310 | 21579360 |
| chr8 | 21824766 | 21824816 |
| chr11 | 122870912 | 122870962 |
| chr8 | 24982546 | 24982596 |
| chr11 | 123811680 | 123811730 |
| chr8 | 30163171 | 30163221 |
| chr11 | 124112011 | 124112061 |
| chr8 | 33574521 | 33574571 |
| chr8 | 34442947 | 34442997 |
| chr8 | 34810262 | 34810312 |
| chr8 | 35009474 | 35009524 |
| chr8 | 39214928 | 39214978 |
| chr8 | 40372107 | 40372157 |
| chr8 | 42792417 | 42792467 |
| chr8 | 43053908 | 43053958 |
| chr8 | 43293109 | 43293159 |
| chr8 | 43561357 | 43561407 |
| chr8 | 48066608 | 48066658 |
| chr8 | 49111125 | 49111175 |
| chr8 | 50004901 | 50004951 |
| chr8 | 52122748 | 52122798 |
| chr8 | 52681243 | 52681293 |
| chr8 | 53536435 | 53536485 |
| chr8 | 55049110 | 55049160 |
| chr8 | 57235757 | 57235807 |
| chr8 | 57983608 | 57983658 |
| chr8 | 58034874 | 58034924 |
| chr8 | 59639792 | 59639842 |
| chr8 | 61512102 | 61512152 |
| chr8 | 62716767 | 62716817 |
| chr8 | 63357664 | 63357714 |
| chr8 | 64143646 | 64143696 |
| chr8 | 65647631 | 65647681 |
| chr8 | 66578304 | 66578354 |
| chr8 | 67193684 | 67193734 |
| chr8 | 68130536 | 68130586 |
| chr8 | 68272211 | 68272261 |
| chr8 | 68274453 | 68274503 |
| chr8 | 68423058 | 68423108 |
| chr8 | 70817719 | 70817769 |
| chr8 | 74807040 | 74807090 |
| chr8 | 87359490 | 87359540 |
| chr8 | 87392904 | 87392954 |
| chr8 | 90279395 | 90279445 |
| chr8 | 94007310 | 94007360 |
| chr8 | 94802987 | 94803037 |
| chr8 | 94939631 | 94939681 |
| chr8 | 95309660 | 95309710 |
| chr8 | 98573994 | 98574044 |
| chr8 | 98809396 | 98809446 |
| chr8 | 99021152 | 99021202 |
| chr8 | 99998632 | 99998682 |
| chr8 | 100896929 | 100896979 |
| chr8 | 101860209 | 101860259 |
| chr8 | 103898175 | 103898225 |
| chr11 | 128537274 | 128537324 |
| chr8 | 117267367 | 117267417 |
| chr8 | 117649872 | 117649922 |
| chr8 | 118395610 | 118395660 |
| chr8 | 118745802 | 118745852 |
| chr8 | 122626152 | 122626202 |
| chr11 | 129254868 | 129254918 |
| chr8 | 124283125 | 124283175 |
| chr8 | 124285606 | 124285656 |
| chr8 | 125401788 | 125401838 |
| chr8 | 128598924 | 128598974 |
| chr8 | 129088391 | 129088441 |
| chr8 | 129088470 | 129088520 |
| chr8 | 129796003 | 129796053 |
| chr8 | 131891567 | 131891617 |
| chr8 | 134725470 | 134725520 |
| chr8 | 143972289 | 143972339 |
| chr11 | 130492741 | 130492791 |
| chr8 | 144001172 | 144001222 |
| chr9 | 5422566 | 5422616 |
| chr9 | 5424193 | 5424243 |
| chr9 | 6612209 | 6612259 |
| chr9 | 6796826 | 6796876 |
| chr9 | 6939844 | 6939894 |
| chr9 | 10438754 | 10438804 |
| chr9 | 15433194 | 15433244 |
| chr9 | 16966537 | 16966587 |
| chr9 | 19423098 | 19423148 |
| chr9 | 26205941 | 26205991 |
| chr9 | 26482072 | 26482122 |
| chr9 | 26654616 | 26654666 |
| chr9 | 27332388 | 27332438 |
| chr9 | 28140416 | 28140466 |
| chr9 | 29180047 | 29180097 |
| chr9 | 32683725 | 32683775 |
| chr9 | 35173889 | 35173939 |
| chr9 | 35174621 | 35174671 |
| chr9 | 37881172 | 37881222 |
| chr9 | 67871577 | 67871627 |
| chr9 | 70879296 | 70879346 |
| chr9 | 70914608 | 70914658 |
| chr9 | 70923881 | 70923931 |
| chr9 | 75026755 | 75026805 |
| chr9 | 75133827 | 75133877 |
| chr9 | 83645073 | 83645123 |
| chr9 | 84383507 | 84383557 |
| chr9 | 85832215 | 85832265 |
| chr9 | 86318262 | 86318312 |
| chr9 | 87064208 | 87064258 |
| chr9 | 90209393 | 90209443 |
| chr9 | 90331814 | 90331864 |
| chr9 | 90332049 | 90332099 |
| chr9 | 90933510 | 90933560 |
| chr9 | 94849984 | 94850034 |
| chr9 | 94850112 | 94850162 |
| chr9 | 94858637 | 94858687 |
| chr9 | 95641048 | 95641098 |
| chr9 | 95641130 | 95641180 |
| chr9 | 95938036 | 95938086 |
| chr9 | 96605676 | 96605726 |
| chr9 | 96605872 | 96605922 |
| chr9 | 98209195 | 98209245 |
| chr9 | 98534277 | 98534327 |
| chr9 | 98534441 | 98534491 |
| chr9 | 100745241 | 100745291 |
| chr9 | 100746438 | 100746488 |
| chr9 | 102377741 | 102377791 |
| chr9 | 102458408 | 102458458 |
| chr9 | 102631264 | 102631314 |
| chr9 | 105400664 | 105400714 |
| chr9 | 105413194 | 105413244 |
| chr9 | 105920431 | 105920481 |
| chr9 | 108240280 | 108240330 |
| chr9 | 109760450 | 109760500 |
| chr9 | 110658031 | 110658081 |
| chr9 | 114979963 | 114980013 |
| chr9 | 116817455 | 116817505 |
| chr9 | 118635803 | 118635853 |
| chr9 | 118775000 | 118775050 |
| chr9 | 120989652 | 120989702 |
| chr9 | 121196388 | 121196438 |
| chr9 | 123467552 | 123467602 |
| chr9 | 123598178 | 123598228 |
| chr9 | 123716865 | 123716915 |
| chr9 | 128658826 | 128658876 |
| chr9 | 130058904 | 130058954 |
| chr9 | 131533811 | 131533861 |
| chr9 | 131952600 | 131952650 |
| chr9 | 132102501 | 132102551 |
| chr9 | 132283480 | 132283530 |
| chr10 | 21723109 | 21723159 |
| chr12 | 4669323 | 4669373 |
| chr12 | 6455793 | 6455843 |
| chr12 | 7242914 | 7242964 |
| chr12 | 8496022 | 8496072 |
| chr12 | 8668093 | 8668143 |
| chr12 | 9392903 | 9392953 |
| chr12 | 9995855 | 9995905 |
| chr12 | 9995804 | 9995854 |
| chr12 | 11502542 | 11502592 |
| chr12 | 12459818 | 12459868 |
| chr12 | 12663361 | 12663411 |
| chr12 | 14398174 | 14398224 |
| chr12 | 19384479 | 19384529 |
| chr12 | 24718263 | 24718313 |
| chr12 | 24971168 | 24971218 |
| chr12 | 28246451 | 28246501 |
| chr12 | 32608405 | 32608455 |
| chr12 | 37009491 | 37009541 |
| chr12 | 39034079 | 39034129 |
| chr12 | 41031030 | 41031080 |
| chr12 | 44265768 | 44265818 |
| chr12 | 44681193 | 44681243 |
| chr12 | 45850566 | 45850616 |
| chr12 | 47181585 | 47181635 |
| chr12 | 47201642 | 47201692 |
| chr12 | 47322573 | 47322623 |
| chr12 | 47596738 | 47596788 |
| chr12 | 47963728 | 47963778 |
| chr12 | 47963802 | 47963852 |
| chr12 | 48115392 | 48115442 |
| chr12 | 50324537 | 50324587 |
| chr12 | 50326226 | 50326276 |
| chr12 | 51090801 | 51090851 |
| chr12 | 53168476 | 53168526 |
| chr12 | 54160277 | 54160327 |
| chr12 | 54329295 | 54329345 |
| chr12 | 54756803 | 54756853 |
| chr12 | 55332429 | 55332479 |
| chr12 | 55464978 | 55465028 |
| chr12 | 55470652 | 55470702 |
| chr12 | 55560307 | 55560357 |
| chr10 | 27590438 | 27590488 |
| chr12 | 56176032 | 56176082 |
| chr12 | 56188739 | 56188789 |
| chr12 | 56344607 | 56344657 |
| chr12 | 60690212 | 60690262 |
| chr12 | 60691842 | 60691892 |
| chr12 | 64840874 | 64840924 |
| chr12 | 64841114 | 64841164 |
| chr12 | 64854457 | 64854507 |
| chr12 | 68438754 | 68438804 |
| chr12 | 68486670 | 68486720 |
| chr12 | 68881105 | 68881155 |
| chr12 | 73269005 | 73269055 |
| chr10 | 29065758 | 29065808 |
| chr10 | 29227381 | 29227431 |
| chr12 | 88400938 | 88400988 |
| chr12 | 88764115 | 88764165 |
| chr12 | 89306818 | 89306868 |
| chr12 | 94132693 | 94132743 |
| chr12 | 94738767 | 94738817 |
| chr12 | 94804907 | 94804957 |
| chr12 | 97551979 | 97552029 |
| chr12 | 99192185 | 99192235 |
| chr12 | 100986007 | 100986057 |
| chr12 | 101147487 | 101147537 |
| chr12 | 102540024 | 102540074 |
| chr12 | 103218909 | 103218959 |
| chr12 | 107316726 | 107316776 |
| chr12 | 107551344 | 107551394 |
| chr12 | 109715997 | 109716047 |
| chr12 | 109801604 | 109801654 |
| chr12 | 110573855 | 110573905 |
| chr12 | 112721510 | 112721560 |
| chr10 | 31655050 | 31655100 |
| chr12 | 116581655 | 116581705 |
| chr12 | 118210960 | 118211010 |
| chr10 | 32264631 | 32264681 |
| chr10 | 32271413 | 32271463 |
| chr12 | 119127047 | 119127097 |
| chr12 | 119128750 | 119128800 |
| chr10 | 32525560 | 32525610 |
| chr10 | 32793443 | 32793493 |
| chr12 | 123891250 | 123891300 |
| chr10 | 34645164 | 34645214 |
| chr12 | 129835954 | 129836004 |
| chr10 | 35269148 | 35269198 |
| chr13 | 18150467 | 18150517 |
| chr13 | 18153150 | 18153200 |
| chr10 | 35655860 | 35655910 |
| chr13 | 19819222 | 19819272 |
| chr13 | 23392028 | 23392078 |
| chr13 | 23853347 | 23853397 |
| chr13 | 23886147 | 23886197 |
| chr13 | 25096987 | 25097037 |
| chr13 | 25178527 | 25178577 |
| chr13 | 28641011 | 28641061 |
| chr13 | 30373477 | 30373527 |
| chr13 | 31087728 | 31087778 |
| chr13 | 31094474 | 31094524 |
| chr13 | 31614992 | 31615042 |
| chr13 | 32911782 | 32911832 |
| chr13 | 32913879 | 32913929 |
| chr13 | 35651609 | 35651659 |
| chr13 | 36736001 | 36736051 |
| chr13 | 39943337 | 39943387 |
| chr13 | 39945132 | 39945182 |
| chr13 | 40253388 | 40253438 |
| chr13 | 40848367 | 40848417 |
| chr13 | 41337783 | 41337833 |
| chr13 | 41788073 | 41788123 |
| chr13 | 43053772 | 43053822 |
| chr13 | 44535964 | 44536014 |
| chr13 | 44887929 | 44887979 |
| chr13 | 47937126 | 47937176 |
| chr13 | 48133222 | 48133272 |
| chr13 | 50451281 | 50451331 |
| chr13 | 51191001 | 51191051 |
| chr10 | 42625880 | 42625930 |
| chr13 | 71948442 | 71948492 |
| chr13 | 74667316 | 74667366 |
| chr13 | 79474134 | 79474184 |
| chr13 | 79518935 | 79518985 |
| chr13 | 83712078 | 83712128 |
| chr10 | 43021917 | 43021967 |
| chr13 | 85911036 | 85911086 |
| chr13 | 86327920 | 86327970 |
| chr13 | 94042622 | 94042672 |
| chr13 | 94196148 | 94196198 |
| chr13 | 94279680 | 94279730 |
| chr13 | 96125101 | 96125151 |
| chr10 | 43202584 | 43202634 |
| chr10 | 43226578 | 43226628 |
| chr13 | 97593377 | 97593427 |
| chr13 | 106132342 | 106132392 |
| chr13 | 108957655 | 108957705 |
| chr13 | 112443952 | 112444002 |
| chr10 | 45125753 | 45125803 |
| chr10 | 45127879 | 45127929 |
| chr14 | 18670218 | 18670268 |
| chr14 | 18901435 | 18901485 |
| chr14 | 19646447 | 19646497 |
| chr10 | 45290795 | 45290845 |
| chr14 | 20156340 | 20156390 |
| chr14 | 20559441 | 20559491 |
| chr14 | 21098152 | 21098202 |
| chr14 | 21567514 | 21567564 |
| chr14 | 22195455 | 22195505 |
| chr14 | 22358544 | 22358594 |
| chr14 | 29897533 | 29897583 |
| chr14 | 30018612 | 30018662 |
| chr14 | 30587748 | 30587798 |
| chr14 | 32444317 | 32444367 |
| chr14 | 33497728 | 33497778 |
| chr14 | 33612065 | 33612115 |
| chr14 | 34437910 | 34437960 |
| chr14 | 36015784 | 36015834 |
| chr14 | 37745688 | 37745738 |
| chr14 | 40134862 | 40134912 |
| chr14 | 43059916 | 43059966 |
| chr14 | 43848168 | 43848218 |
| chr14 | 43850302 | 43850352 |
| chr14 | 44094152 | 44094202 |
| chr14 | 44094745 | 44094795 |
| chr14 | 48277208 | 48277258 |
| chr14 | 49414281 | 49414331 |
| chr14 | 49823063 | 49823113 |
| chr14 | 49924157 | 49924207 |
| chr14 | 52659173 | 52659223 |
| chr14 | 53913324 | 53913374 |
| chr14 | 54376795 | 54376845 |
| chr14 | 61621604 | 61621654 |
| chr14 | 61980535 | 61980585 |
| chr14 | 62495720 | 62495770 |
| chr14 | 62640744 | 62640794 |
| chr14 | 63353560 | 63353610 |
| chr14 | 64150557 | 64150607 |
| chr14 | 65002647 | 65002697 |
| chr14 | 68359326 | 68359376 |
| chr14 | 68933729 | 68933779 |
| chr14 | 69834765 | 69834815 |
| chr14 | 70693616 | 70693666 |
| chr14 | 71642570 | 71642620 |
| chr10 | 52497811 | 52497861 |
| chr10 | 52497995 | 52498045 |
| chr14 | 75277927 | 75277977 |
| chr14 | 76204463 | 76204513 |
| chr14 | 76450964 | 76451014 |
| chr14 | 79701595 | 79701645 |
| chr14 | 79919736 | 79919786 |
| chr14 | 81271688 | 81271738 |
| chr14 | 82555168 | 82555218 |
| chr14 | 83809998 | 83810048 |
| chr14 | 84287070 | 84287120 |
| chr14 | 90014834 | 90014884 |
| chr14 | 90644482 | 90644532 |
| chr14 | 91369269 | 91369319 |
| chr14 | 93115455 | 93115505 |
| chr14 | 100629249 | 100629299 |
| chr10 | 59348591 | 59348641 |
| chr15 | 24812598 | 24812648 |
| chr15 | 29192091 | 29192141 |
| chr15 | 32954594 | 32954644 |
| chr15 | 33663776 | 33663826 |
| chr15 | 37903549 | 37903599 |
| chr15 | 39392511 | 39392561 |
| chr15 | 39970716 | 39970766 |
| chr10 | 64033294 | 64033344 |
| chr10 | 64053500 | 64053550 |
| chr15 | 40609868 | 40609918 |
| chr15 | 41176502 | 41176552 |
| chr15 | 43302398 | 43302448 |
| chr15 | 44107034 | 44107084 |
| chr15 | 47559523 | 47559573 |
| chr15 | 48182218 | 48182268 |
| chr15 | 49893233 | 49893283 |
| chr15 | 50468909 | 50468959 |
| chr15 | 52790412 | 52790462 |
| chr15 | 53453930 | 53453980 |
| chr10 | 64915612 | 64915662 |
| chr10 | 64916068 | 64916118 |
| chr10 | 64916861 | 64916911 |
| chr15 | 54549117 | 54549167 |
| chr15 | 60227200 | 60227250 |
| chr15 | 60341617 | 60341667 |
| chr10 | 66155772 | 66155822 |
| chr10 | 66155932 | 66155982 |
| chr15 | 60699306 | 60699356 |
| chr15 | 61106455 | 61106505 |
| chr15 | 66141657 | 66141707 |
| chr15 | 66866763 | 66866813 |
| chr15 | 66869293 | 66869343 |
| chr15 | 66973811 | 66973861 |
| chr15 | 68880567 | 68880617 |
| chr10 | 68795183 | 68795233 |
| chr15 | 69776945 | 69776995 |
| chr15 | 72738079 | 72738129 |
| chr15 | 73488837 | 73488887 |
| chr15 | 78655892 | 78655942 |
| chr10 | 69383639 | 69383689 |
| chr15 | 82211845 | 82211895 |
| chr15 | 82945019 | 82945069 |
| chr10 | 2803222 | 2803272 |
| chr15 | 86798037 | 86798087 |
| chr15 | 88126493 | 88126543 |
| chr15 | 88139945 | 88139995 |
| chr15 | 88142660 | 88142710 |
| chr15 | 90880648 | 90880698 |
| chr15 | 91042469 | 91042519 |
| chr15 | 99050468 | 99050518 |
| chr16 | 1588642 | 1588692 |
| chr16 | 2210094 | 2210144 |
| chr16 | 2390927 | 2390977 |
| chr16 | 2853159 | 2853209 |
| chr16 | 3321835 | 3321885 |
| chr10 | 71098249 | 71098299 |
| chr10 | 71098476 | 71098526 |
| chr16 | 7952462 | 7952512 |
| chr16 | 9411886 | 9411936 |
| chr16 | 11396349 | 11396399 |
| chr16 | 13949321 | 13949371 |
| chr10 | 71512902 | 71512952 |
| chr16 | 14593842 | 14593892 |
| chr10 | 71734796 | 71734846 |
| chr10 | 71793315 | 71793365 |
| chr10 | 71793421 | 71793471 |
| chr16 | 30216836 | 30216886 |
| chr16 | 31150915 | 31150965 |
| chr16 | 31153208 | 31153258 |
| chr16 | 31279128 | 31279178 |
| chr16 | 31883300 | 31883350 |
| chr16 | 46469163 | 46469213 |
| chr16 | 46509889 | 46509939 |
| chr16 | 46814287 | 46814337 |
| chr16 | 47871451 | 47871501 |
| chr16 | 48227930 | 48227980 |
| chr16 | 48295737 | 48295787 |
| chr16 | 48762926 | 48762976 |
| chr16 | 49023177 | 49023227 |
| chr10 | 72981158 | 72981208 |
| chr10 | 72981303 | 72981353 |
| chr16 | 53350948 | 53350998 |
| chr16 | 53437819 | 53437869 |
| chr16 | 53510930 | 53510980 |
| chr16 | 53512182 | 53512232 |
| chr16 | 55799000 | 55799050 |
| chr16 | 56550325 | 56550375 |
| chr16 | 57011286 | 57011336 |
| chr16 | 58207131 | 58207181 |
| chr16 | 58656372 | 58656422 |
| chr16 | 60167127 | 60167177 |
| chr16 | 66324250 | 66324300 |
| chr16 | 67974562 | 67974612 |
| chr16 | 69861995 | 69862045 |
| chr16 | 71398847 | 71398897 |
| chr16 | 72306832 | 72306882 |
| chr16 | 76313675 | 76313725 |
| chr16 | 80058285 | 80058335 |
| chr10 | 74820058 | 74820108 |
| chr16 | 89272919 | 89272969 |
| chr17 | 1800608 | 1800658 |
| chr17 | 2616089 | 2616139 |
| chr17 | 3221653 | 3221703 |
| chr17 | 3482778 | 3482828 |
| chr10 | 76438594 | 76438644 |
| chr10 | 76855725 | 76855775 |
| chr17 | 5825875 | 5825925 |
| chr17 | 8476946 | 8476996 |
| chr17 | 8655083 | 8655133 |
| chr17 | 10160440 | 10160490 |
| chr17 | 13770226 | 13770276 |
| chr17 | 16084032 | 16084082 |
| chr10 | 79180836 | 79180886 |
| chr17 | 18992705 | 18992755 |
| chr10 | 79574109 | 79574159 |
| chr17 | 19694535 | 19694585 |
| chr17 | 19750158 | 19750208 |
| chr17 | 20623429 | 20623479 |
| chr17 | 21049199 | 21049249 |
| chr17 | 25683858 | 25683908 |
| chr17 | 26929269 | 26929319 |
| chr17 | 27077855 | 27077905 |
| chr17 | 27132707 | 27132757 |
| chr17 | 28005198 | 28005248 |
| chr17 | 28996952 | 28997002 |
| chr17 | 28995257 | 28995307 |
| chr17 | 29009873 | 29009923 |
| chr17 | 29209120 | 29209170 |
| chr17 | 29453937 | 29453987 |
| chr10 | 80384278 | 80384328 |
| chr17 | 31396394 | 31396444 |
| chr17 | 33668878 | 33668928 |
| chr17 | 36120972 | 36121022 |
| chr17 | 37161394 | 37161444 |
| chr17 | 37462476 | 37462526 |

Table 15DY. Repeat Category K.standard.SVA_A

| | | |
|---|---|---|
| chr17 | 56761362 | 56761412 |
| chr18 | 9420600 | 9420650 |
| chr18 | 10497097 | 10497147 |
| chr18 | 11604243 | 11604293 |
| chr18 | 11638686 | 11638736 |
| chr18 | 12233072 | 12233122 |
| chr18 | 14211912 | 14211962 |
| chr18 | 16970446 | 16970496 |
| chr10 | 97234931 | 97234981 |
| chr18 | 46197026 | 46197076 |
| chr18 | 65919396 | 65919446 |
| chr10 | 101265324 | 101265374 |
| chr19 | 21234798 | 21234848 |
| chr19 | 21236700 | 21236750 |
| chr19 | 35085381 | 35085431 |
| chr19 | 43990271 | 43990321 |
| chr19 | 50840274 | 50840324 |
| chr19 | 55777747 | 55777797 |
| chr19 | 57914686 | 57914736 |
| chr1 | 30321610 | 30321660 |
| chr1 | 33018906 | 33018956 |
| chr1 | 39879966 | 39880016 |
| chr1 | 44983957 | 44984007 |
| chr1 | 45572936 | 45572986 |
| chr1 | 46075784 | 46075834 |
| chr1 | 99436588 | 99436638 |
| chr1 | 99436839 | 99436889 |
| chr1 | 112569776 | 112569826 |
| chr1 | 114084359 | 114084409 |
| chr1 | 115199896 | 115199946 |
| chr1 | 141723087 | 141723137 |
| chr1 | 149267901 | 149267951 |
| chr1 | 151568711 | 151568761 |
| chr1 | 157717997 | 157718047 |
| chr1 | 159608960 | 159609010 |
| chr1 | 160655110 | 160655160 |
| chr1 | 160656709 | 160656759 |
| chr1 | 164928750 | 164928800 |
| chr1 | 164963491 | 164963541 |
| chr1 | 171969996 | 171970046 |
| chr1 | 180635497 | 180635547 |
| chr1 | 184823767 | 184823817 |
| chr1 | 195841881 | 195841931 |
| chr1 | 222959363 | 222959413 |
| chr1 | 235530678 | 235530728 |
| chr20 | 3102191 | 3102241 |
| chr20 | 9361136 | 9361186 |
| chr21 | 14227516 | 14227566 |
| chr21 | 15966999 | 15967049 |
| chr21 | 18015648 | 18015698 |
| chr21 | 29684534 | 29684584 |
| chr11 | 508667 | 508717 |
| chr22 | 37620291 | 37620341 |
| chr22 | 37620133 | 37620183 |
| chr22 | 37650110 | 37650160 |
| chr22 | 48610304 | 48610354 |
| chrX | 22902646 | 22902696 |
| chrX | 26838354 | 26838404 |
| chrX | 54365148 | 54365198 |
| chrX | 68436975 | 68437025 |
| chrX | 69387244 | 69387294 |
| chrX | 69386927 | 69386977 |
| chrX | 69761106 | 69761156 |
| chrX | 69762844 | 69762894 |
| chrX | 71107436 | 71107486 |
| chrX | 71109494 | 71109544 |
| chrX | 73420418 | 73420468 |
| chrX | 73422875 | 73422925 |
| chrX | 75153113 | 75153163 |
| chrX | 99766108 | 99766158 |
| chrX | 120487623 | 120487673 |
| chrX | 131324699 | 131324749 |
| chrY | 13574876 | 13574926 |
| chrY | 21559578 | 21559628 |
| chr2 | 24598129 | 24598179 |
| chr2 | 25732866 | 25732916 |
| chr2 | 25734107 | 25734157 |
| chr2 | 27792529 | 27792579 |
| chr2 | 44263336 | 44263386 |
| chr2 | 86548260 | 86548310 |
| chr2 | 86748035 | 86748085 |
| chr2 | 86751223 | 86751273 |
| chr2 | 98340411 | 98340461 |
| chr2 | 170387761 | 170387811 |
| chr11 | 37418083 | 37418133 |
| chr2 | 188705461 | 188705511 |
| chr2 | 204134699 | 204134749 |
| chr2 | 232605106 | 232605156 |
| chr2 | 243320065 | 243320115 |
| chr3 | 41843453 | 41843503 |
| chr3 | 72058973 | 72059023 |
| chr3 | 75369099 | 75369149 |
| chr3 | 95084603 | 95084653 |
| chr3 | 102591681 | 102591731 |
| chr3 | 158218436 | 158218486 |
| chr4 | 305760 | 305810 |
| chr4 | 28268326 | 28268376 |
| chr4 | 41408307 | 41408357 |
| chr4 | 41409959 | 41410009 |
| chr4 | 45126888 | 45126938 |
| chr4 | 68249466 | 68249516 |
| chr4 | 92548149 | 92548199 |
| chr4 | 99710973 | 99711023 |
| chr4 | 106881271 | 106881321 |
| chr4 | 111057630 | 111057680 |
| chr4 | 111059573 | 111059623 |
| chr4 | 160257882 | 160257932 |
| chr5 | 32085228 | 32085278 |
| chr5 | 32088284 | 32088334 |
| chr5 | 37565317 | 37565367 |
| chr11 | 70029414 | 70029464 |
| chr5 | 49919632 | 49919682 |
| chr5 | 50087211 | 50087261 |
| chr5 | 56830192 | 56830242 |
| chr5 | 57876806 | 57876856 |
| chr5 | 63537275 | 63537325 |
| chr5 | 74991734 | 74991784 |
| chr5 | 77140242 | 77140292 |
| chr5 | 82817681 | 82817731 |
| chr5 | 100198261 | 100198311 |
| chr5 | 133269558 | 133269608 |
| chr11 | 78316902 | 78316952 |
| chr6 | 30296612 | 30296662 |
| chr6 | 33372672 | 33372722 |
| chr6 | 57222629 | 57222679 |
| chr6 | 63870774 | 63870824 |
| chr6 | 69827387 | 69827437 |
| chr6 | 70137007 | 70137057 |
| chr6 | 70139399 | 70139449 |
| chr6 | 158678351 | 158678401 |
| chr6 | 170013381 | 170013431 |
| chr7 | 6007030 | 6007080 |
| chr7 | 25957311 | 25957361 |
| chr7 | 25959456 | 25959506 |
| chr11 | 111614658 | 111614708 |
| chr7 | 30935952 | 30936002 |
| chr7 | 33086237 | 33086287 |
| chr11 | 112931818 | 112931868 |
| chr7 | 44652531 | 44652581 |
| chr7 | 54252876 | 54252926 |
| chr7 | 66959479 | 66959529 |
| chr7 | 76778790 | 76778840 |
| chr7 | 91129839 | 91129889 |
| chr7 | 91267518 | 91267568 |
| chr7 | 94090765 | 94090815 |
| chr7 | 96401185 | 96401235 |
| chr7 | 99736285 | 99736335 |
| chr7 | 102270270 | 102270320 |
| chr7 | 115832583 | 115832633 |
| chr7 | 128409087 | 128409137 |
| chr7 | 128410119 | 128410169 |
| chr7 | 128856947 | 128856997 |
| chr7 | 138323984 | 138324034 |
| chr7 | 138325521 | 138325571 |
| chr7 | 154279852 | 154279902 |
| chr10 | 17678461 | 17678511 |
| chr8 | 48066608 | 48066658 |
| chr8 | 53536435 | 53536485 |
| chr8 | 55049110 | 55049160 |
| chr8 | 63357664 | 63357714 |
| chr8 | 70817719 | 70817769 |
| chr8 | 94802987 | 94803037 |
| chr8 | 98809396 | 98809446 |
| chr8 | 118395610 | 118395660 |
| chr9 | 5422566 | 5422616 |
| chr9 | 5424193 | 5424243 |
| chr9 | 26482072 | 26482122 |
| chr9 | 28140416 | 28140466 |
| chr9 | 35173889 | 35173939 |
| chr9 | 35174621 | 35174671 |
| chr9 | 67871577 | 67871627 |
| chr9 | 90209393 | 90209443 |
| chr9 | 96605676 | 96605726 |
| chr9 | 96605872 | 96605922 |
| chr9 | 98533965 | 98534015 |
| chr9 | 98534277 | 98534327 |
| chr9 | 98534441 | 98534491 |
| chr9 | 102377741 | 102377791 |
| chr9 | 102458408 | 102458458 |
| chr9 | 132324195 | 132324245 |
| chr12 | 11502542 | 11502592 |
| chr12 | 40906831 | 40906881 |
| chr12 | 44265768 | 44265818 |
| chr12 | 44681193 | 44681243 |
| chr12 | 47322573 | 47322623 |
| chr12 | 47525993 | 47526043 |
| chr12 | 60690212 | 60690262 |
| chr12 | 60691842 | 60691892 |
| chr12 | 64840874 | 64840924 |
| chr12 | 64841114 | 64841164 |
| chr12 | 64854457 | 64854507 |
| chr12 | 68486670 | 68486720 |
| chr12 | 100986007 | 100986057 |
| chr10 | 31655050 | 31655100 |
| chr12 | 119127047 | 119127097 |
| chr12 | 119128750 | 119128800 |
| chr12 | 119129274 | 119129324 |
| chr10 | 32800211 | 32800261 |
| chr13 | 17206811 | 17206861 |
| chr10 | 35655860 | 35655910 |
| chr13 | 40848367 | 40848417 |
| chr13 | 41788073 | 41788123 |
| chr13 | 76031516 | 76031566 |
| chr10 | 43021917 | 43021967 |
| chr13 | 88282518 | 88282568 |
| chr13 | 96125101 | 96125151 |
| chr10 | 45125753 | 45125803 |
| chr10 | 45127879 | 45127929 |
| chr14 | 20559441 | 20559491 |
| chr14 | 21567514 | 21567564 |
| chr14 | 44094152 | 44094202 |
| chr14 | 44094745 | 44094795 |
| chr14 | 49823063 | 49823113 |
| chr14 | 64172573 | 64172623 |
| chr14 | 90644482 | 90644532 |
| chr10 | 59348591 | 59348641 |
| chr14 | 103553155 | 103553205 |
| chr10 | 64033294 | 64033344 |
| chr15 | 53453930 | 53453980 |
| chr15 | 54549117 | 54549167 |
| chr16 | 2212473 | 2212523 |
| chr16 | 2213402 | 2213452 |
| chr16 | 2214399 | 2214449 |
| chr16 | 2216275 | 2216325 |
| chr16 | 9411886 | 9411936 |
| chr10 | 71345498 | 71345548 |
| chr16 | 47871451 | 47871501 |
| chr16 | 55799000 | 55799050 |
| chr16 | 60167127 | 60167177 |
| chr17 | 8480455 | 8480505 |
| chr17 | 13770226 | 13770276 |
| chr17 | 27132707 | 27132757 |

Table 15DZ. Repeat Category K.standard.SVA_B

| | | |
|---|---|---|
| chr17 | 37864620 | 37864670 |
| chr10 | 85605701 | 85605751 |
| chr17 | 56397987 | 56398037 |
| chr17 | 59528917 | 59528967 |
| chr17 | 64382861 | 64382911 |
| chr10 | 87878662 | 87878712 |
| chr17 | 81411705 | 81411755 |
| chr18 | 28980431 | 28980481 |
| chr18 | 43605810 | 43605860 |
| chr18 | 46387762 | 46387812 |
| chr18 | 74933112 | 74933162 |
| chr19 | 11649906 | 11649956 |
| chr19 | 12333644 | 12333694 |
| chr10 | 103726614 | 103726664 |
| chr19 | 21023513 | 21023563 |
| chr19 | 21188097 | 21188147 |
| chr19 | 21419107 | 21419157 |
| chr19 | 21589414 | 21589464 |
| chr19 | 22296495 | 22296545 |
| chr19 | 22675294 | 22675344 |
| chr19 | 22677851 | 22677901 |
| chr19 | 22900009 | 22900059 |
| chr19 | 23136141 | 23136191 |
| chr19 | 23265473 | 23265523 |
| chr19 | 49398958 | 49399008 |
| chr19 | 55586783 | 55586833 |
| chr19 | 60112838 | 60112888 |
| chr1 | 32774148 | 32774198 |
| chr1 | 33325049 | 33325099 |
| chr1 | 34050727 | 34050777 |
| chr1 | 34840779 | 34840829 |
| chr1 | 39748084 | 39748134 |
| chr1 | 44995084 | 44995134 |
| chr1 | 45126460 | 45126510 |
| chr1 | 45602161 | 45602211 |
| chr1 | 50787686 | 50787736 |
| chr1 | 80923529 | 80923579 |
| chr1 | 91221796 | 91221846 |
| chr1 | 91964013 | 91964063 |
| chr1 | 91964210 | 91964260 |
| chr1 | 91964585 | 91964635 |
| chr1 | 117190189 | 117190239 |
| chr1 | 144039781 | 144039831 |
| chr1 | 148958259 | 148958309 |
| chr1 | 149334791 | 149334841 |
| chr10 | 127233754 | 127233804 |
| chr10 | 128266789 | 128266839 |
| chr1 | 158119199 | 158119249 |
| chr1 | 158216159 | 158216209 |
| chr1 | 160702335 | 160702385 |
| chr1 | 164186577 | 164186627 |
| chr1 | 165993319 | 165993369 |
| chr1 | 186397756 | 186397806 |
| chr1 | 200524238 | 200524288 |
| chr1 | 200525208 | 200525258 |
| chr1 | 200987630 | 200987680 |
| chr1 | 209243993 | 209244043 |
| chr1 | 221512399 | 221512449 |
| chr1 | 222777223 | 222777273 |
| chr1 | 228624252 | 228624302 |
| chr1 | 233632164 | 233632214 |
| chr1 | 242885219 | 242885269 |
| chr20 | 36464063 | 36464113 |
| chr20 | 37826042 | 37826092 |
| chr21 | 25989075 | 25989125 |
| chr21 | 36684328 | 36684378 |
| chr21 | 44096750 | 44096800 |
| chr10 | 9320215 | 9320265 |
| chr22 | 29905657 | 29905707 |
| chr10 | 9901903 | 9901953 |
| chr22 | 41235579 | 41235629 |
| chr22 | 41599274 | 41599324 |
| chrX | 12992230 | 12992280 |
| chr11 | 4222421 | 4222471 |
| chr11 | 4322037 | 4322087 |
| chrX | 25284717 | 25284767 |
| chr11 | 5945739 | 5945789 |
| chrX | 44410035 | 44410085 |
| chrX | 49779322 | 49779372 |
| chrX | 52714800 | 52714850 |
| chrX | 53208387 | 53208437 |
| chrX | 53729273 | 53729323 |
| chrX | 61913176 | 61913226 |
| chrX | 64062729 | 64062779 |
| chrX | 67508653 | 67508703 |
| chr11 | 8508575 | 8508625 |
| chrX | 68209070 | 68209120 |
| chrX | 69732487 | 69732537 |
| chrX | 71968944 | 71968994 |
| chrX | 85060151 | 85060201 |
| chrX | 132126485 | 132126535 |
| chrX | 136071210 | 136071260 |
| chrX | 143798909 | 143798959 |
| chrX | 149386401 | 149386451 |
| chrX | 152875190 | 152875240 |
| chrY | 3866599 | 3866649 |
| chrY | 20592134 | 20592184 |
| chr11 | 15672337 | 15672387 |
| chr2 | 26863096 | 26863146 |
| chr2 | 34333508 | 34333558 |
| chr2 | 38179276 | 38179326 |
| chr2 | 50614095 | 50614145 |
| chr2 | 70469813 | 70469863 |
| chr2 | 74325177 | 74325227 |
| chr2 | 89122948 | 89122998 |
| chr11 | 31957916 | 31957966 |
| chr2 | 110558671 | 110558721 |
| chr2 | 126130693 | 126130743 |
| chr2 | 130659025 | 130659075 |
| chr2 | 136243700 | 136243750 |
| chr2 | 159370579 | 159370629 |
| chr2 | 173767165 | 173767215 |
| chr2 | 190720813 | 190720863 |
| chr2 | 193960514 | 193960564 |
| chr2 | 202109174 | 202109224 |
| chr2 | 216231881 | 216231931 |
| chr2 | 220819339 | 220819389 |
| chr10 | 12273291 | 12273341 |
| chr2 | 240172343 | 240172393 |
| chr3 | 2933957 | 2934007 |
| chr3 | 5260197 | 5260247 |
| chr3 | 16013215 | 16013265 |
| chr3 | 20565198 | 20565248 |
| chr3 | 38054848 | 38054898 |
| chr3 | 38067523 | 38067573 |
| chr11 | 47877944 | 47877994 |
| chr3 | 39657831 | 39657881 |
| chr3 | 42912502 | 42912552 |
| chr11 | 48942104 | 48942154 |
| chr3 | 50800243 | 50800293 |
| chr3 | 51601378 | 51601428 |
| chr11 | 56039982 | 56040032 |
| chr3 | 60870782 | 60870832 |
| chr3 | 61336734 | 61336784 |
| chr11 | 57487558 | 57487608 |
| chr3 | 98521242 | 98521292 |
| chr11 | 60052632 | 60052682 |
| chr3 | 114497959 | 114498009 |
| chr3 | 117558333 | 117558383 |
| chr3 | 120282325 | 120282375 |
| chr3 | 128333637 | 128333687 |
| chr3 | 128349536 | 128349586 |
| chr3 | 139561450 | 139561500 |
| chr3 | 139825401 | 139825451 |
| chr3 | 165822502 | 165822552 |
| chr3 | 176126000 | 176126050 |
| chr3 | 180070735 | 180070785 |
| chr3 | 192163079 | 192163129 |
| chr4 | 3127986 | 3128036 |
| chr4 | 6903951 | 6904001 |
| chr4 | 25002162 | 25002212 |
| chr4 | 37406979 | 37407029 |
| chr4 | 48104192 | 48104242 |
| chr4 | 53774448 | 53774498 |
| chr4 | 53922258 | 53922308 |
| chr4 | 71218992 | 71219042 |
| chr4 | 79387535 | 79387585 |
| chr4 | 80625236 | 80625286 |
| chr4 | 83456560 | 83456610 |
| chr4 | 107701499 | 107701549 |
| chr4 | 110983061 | 110983111 |
| chr4 | 115175721 | 115175771 |
| chr4 | 122617937 | 122617987 |
| chr4 | 152452363 | 152452413 |
| chr4 | 152463170 | 152463220 |
| chr4 | 153229381 | 153229431 |
| chr4 | 154264872 | 154264922 |
| chr4 | 159137232 | 159137282 |
| chr4 | 171296972 | 171297022 |
| chr4 | 171446308 | 171446358 |
| chr4 | 171784156 | 171784206 |
| chr4 | 174800413 | 174800463 |
| chr5 | 43712487 | 43712537 |
| chr5 | 43792676 | 43792726 |
| chr5 | 43869392 | 43869442 |
| chr5 | 55570573 | 55570623 |
| chr5 | 55710450 | 55710500 |
| chr5 | 75931791 | 75931841 |
| chr5 | 93396334 | 93396384 |
| chr5 | 109245612 | 109245662 |
| chr5 | 111772160 | 111772210 |
| chr5 | 119558610 | 119558660 |
| chr11 | 73765747 | 73765797 |
| chr5 | 156347832 | 156347882 |
| chr5 | 157443683 | 157443733 |
| chr5 | 157445895 | 157445945 |
| chr5 | 162992490 | 162992540 |
| chr11 | 74815284 | 74815334 |
| chr6 | 13241543 | 13241593 |
| chr11 | 78936415 | 78936465 |
| chr6 | 24522959 | 24523009 |
| chr6 | 25695119 | 25695169 |
| chr6 | 27605851 | 27605901 |
| chr6 | 28578886 | 28578936 |
| chr6 | 31142316 | 31142366 |
| chr6 | 31401822 | 31401872 |
| chr6 | 31927680 | 31927730 |
| chr6 | 40830583 | 40830633 |
| chr6 | 42702506 | 42702556 |
| chr6 | 47459498 | 47459548 |
| chr6 | 57959000 | 57959050 |
| chr6 | 58563155 | 58563205 |
| chr6 | 70671601 | 70671651 |
| chr6 | 74631362 | 74631412 |
| chr6 | 114298275 | 114298325 |
| chr6 | 122031226 | 122031276 |
| chr11 | 98179198 | 98179248 |
| chr6 | 144772891 | 144772941 |
| chr6 | 159737485 | 159737535 |
| chr6 | 159738545 | 159738595 |
| chr7 | 1945044 | 1945094 |
| chr11 | 108553805 | 108553855 |
| chr7 | 27770474 | 27770524 |
| chr7 | 32536766 | 32536816 |
| chr7 | 44773074 | 44773124 |
| chr7 | 47701973 | 47702023 |
| chr7 | 76780254 | 76780304 |
| chr7 | 91785287 | 91785337 |
| chr7 | 103911520 | 103911570 |
| chr7 | 107456084 | 107456134 |
| chr7 | 127656826 | 127656876 |
| chr11 | 118400170 | 118400220 |
| chr7 | 137478490 | 137478540 |
| chr7 | 141724082 | 141724132 |
| chr7 | 148422005 | 148422055 |
| chr8 | 15314670 | 15314720 |
| chr8 | 21824766 | 21824816 |
| chr8 | 24982546 | 24982596 |
| chr8 | 34810262 | 34810312 |
| chr8 | 39214928 | 39214978 |
| chr8 | 40372107 | 40372157 |
| chr8 | 40890180 | 40890230 |
| chr8 | 42792417 | 42792467 |
| chr8 | 50004901 | 50004951 |
| chr8 | 59740450 | 59740500 |
| chr8 | 74807040 | 74807090 |
| chr8 | 87359490 | 87359540 |
| chr8 | 90279395 | 90279445 |
| chr8 | 94939631 | 94939681 |
| chr8 | 99021152 | 99021202 |
| chr8 | 101321606 | 101321656 |
| chr11 | 128537274 | 128537324 |
| chr8 | 117267367 | 117267417 |
| chr8 | 118745802 | 118745852 |
| chr8 | 143972289 | 143972339 |
| chr9 | 15433194 | 15433244 |
| chr9 | 26205941 | 26205991 |
| chr9 | 26654616 | 26654666 |
| chr9 | 27332388 | 27332438 |
| chr9 | 75776286 | 75776336 |
| chr9 | 94858637 | 94858687 |
| chr9 | 94858981 | 94859031 |
| chr9 | 94859352 | 94859402 |
| chr9 | 95641048 | 95641098 |
| chr9 | 95641130 | 95641180 |
| chr9 | 100745241 | 100745291 |
| chr9 | 100746438 | 100746488 |
| chr9 | 102631264 | 102631314 |
| chr9 | 116817455 | 116817505 |
| chr9 | 118775000 | 118775050 |
| chr9 | 132283480 | 132283530 |
| chr12 | 12663361 | 12663411 |
| chr12 | 24718263 | 24718313 |
| chr12 | 41031030 | 41031080 |
| chr12 | 45850566 | 45850616 |
| chr12 | 54329295 | 54329345 |
| chr12 | 55560307 | 55560357 |
| chr12 | 56176032 | 56176082 |
| chr12 | 56176903 | 56176953 |
| chr12 | 68056520 | 68056570 |
| chr12 | 68534826 | 68534876 |
| chr12 | 73269005 | 73269055 |
| chr10 | 29065758 | 29065808 |
| chr12 | 101147487 | 101147537 |
| chr12 | 107316726 | 107316776 |
| chr12 | 116581655 | 116581705 |
| chr10 | 32271413 | 32271463 |
| chr12 | 119130771 | 119130821 |
| chr10 | 32525560 | 32525610 |
| chr10 | 32793443 | 32793493 |
| chr10 | 34645164 | 34645214 |
| chr10 | 35269148 | 35269198 |
| chr13 | 31094474 | 31094524 |
| chr13 | 31614992 | 31615042 |
| chr13 | 34340336 | 34340386 |
| chr13 | 36736001 | 36736051 |
| chr13 | 39943337 | 39943387 |
| chr13 | 39945132 | 39945182 |
| chr13 | 51191001 | 51191051 |
| chr13 | 74667316 | 74667366 |
| chr13 | 83712078 | 83712128 |
| chr13 | 85911036 | 85911086 |
| chr13 | 94196148 | 94196198 |
| chr13 | 94279680 | 94279730 |
| chr10 | 43226578 | 43226628 |
| chr10 | 43227149 | 43227199 |
| chr10 | 43227801 | 43227851 |
| chr14 | 18901435 | 18901485 |
| chr14 | 20156340 | 20156390 |
| chr14 | 22195455 | 22195505 |
| chr14 | 22358544 | 22358594 |
| chr14 | 30018612 | 30018662 |
| chr14 | 30587748 | 30587798 |
| chr14 | 32444317 | 32444367 |
| chr14 | 49414281 | 49414331 |
| chr14 | 49415032 | 49415082 |
| chr14 | 49924157 | 49924207 |
| chr14 | 54376795 | 54376845 |
| chr10 | 51461449 | 51461499 |
| chr14 | 69834765 | 69834815 |
| chr14 | 76204463 | 76204513 |
| chr14 | 79701595 | 79701645 |
| chr14 | 79919736 | 79919786 |
| chr15 | 40609868 | 40609918 |
| chr15 | 44107034 | 44107084 |
| chr15 | 49893233 | 49893283 |
| chr15 | 50468909 | 50468959 |
| chr15 | 52790412 | 52790462 |
| chr15 | 68880567 | 68880617 |
| chr15 | 78655892 | 78655942 |
| chr15 | 86798037 | 86798087 |
| chr16 | 13949321 | 13949371 |
| chr16 | 16677540 | 16677590 |
| chr16 | 48227930 | 48227980 |
| chr16 | 48295737 | 48295787 |
| chr16 | 53437819 | 53437869 |
| chr16 | 66324250 | 66324300 |
| chr16 | 76313675 | 76313725 |
| chr10 | 74820058 | 74820108 |
| chr10 | 76855725 | 76855775 |
| chr10 | 76856010 | 76856060 |
| chr17 | 8476946 | 8476996 |
| chr17 | 8479472 | 8479522 |
| chr10 | 79180836 | 79180886 |
| chr17 | 20623429 | 20623479 |
| chr10 | 80384278 | 80384328 |

Table 15EA. Repeat Category K.standard.SVA_C

| | | |
|---|---|---|
| chr17 | 41685175 | 41685225 |
| chr17 | 66711119 | 66711169 |
| chr18 | 11598689 | 11598739 |
| chr18 | 66387013 | 66387063 |
| chr19 | 21681938 | 21681988 |
| chr19 | 22079302 | 22079352 |
| chr19 | 23394402 | 23394452 |
| chr19 | 23508371 | 23508421 |
| chr19 | 23937139 | 23937189 |
| chr19 | 35131898 | 35131948 |
| chr19 | 47836528 | 47836578 |
| chr19 | 47836738 | 47836788 |
| chr19 | 48743784 | 48743834 |
| chr19 | 62687387 | 62687437 |
| chr1 | 11076259 | 11076309 |
| chr1 | 21297955 | 21298005 |
| chr10 | 120985417 | 120985467 |
| chr1 | 86852333 | 86852383 |
| chr1 | 99007962 | 99008012 |
| chr1 | 99008339 | 99008389 |
| chr1 | 99786467 | 99786517 |
| chr1 | 114321118 | 114321168 |
| chr1 | 114646723 | 114646773 |
| chr1 | 143419906 | 143419956 |
| chr1 | 156635625 | 156635675 |
| chr1 | 165365335 | 165365385 |
| chr1 | 168902433 | 168902483 |
| chr1 | 177918084 | 177918134 |
| chr1 | 244632046 | 244632096 |
| chr20 | 5615696 | 5615746 |
| chr20 | 8619097 | 8619147 |
| chr20 | 11489840 | 11489890 |
| chr20 | 18350266 | 18350316 |
| chr20 | 32693778 | 32693828 |
| chr22 | 20356744 | 20356794 |
| chr22 | 28272240 | 28272290 |
| chr22 | 29354386 | 29354436 |
| chrX | 13681062 | 13681112 |
| chrX | 16625550 | 16625600 |
| chrX | 35640551 | 35640601 |
| chrX | 43815528 | 43815578 |
| chrX | 55373737 | 55373787 |
| chrX | 72499987 | 72500037 |
| chrX | 85492630 | 85492680 |
| chrX | 124514656 | 124514706 |
| chr2 | 33961641 | 33961691 |
| chr2 | 38165915 | 38165965 |
| chr2 | 53525354 | 53525404 |
| chr2 | 62832413 | 62832463 |
| chr2 | 183453258 | 183453308 |
| chr2 | 202233824 | 202233874 |
| chr2 | 209445754 | 209445804 |
| chr2 | 212017257 | 212017307 |
| chr2 | 219417030 | 219417080 |
| chr3 | 8342595 | 8342645 |
| chr3 | 41985386 | 41985436 |
| chr3 | 47311155 | 47311205 |
| chr11 | 49785412 | 49785462 |
| chr11 | 50048250 | 50048300 |
| chr3 | 56576597 | 56576647 |
| chr3 | 84888267 | 84888317 |
| chr11 | 60033074 | 60033124 |
| chr11 | 60033643 | 60033693 |
| chr3 | 129812448 | 129812498 |
| chr3 | 138911732 | 138911782 |
| chr3 | 142070326 | 142070376 |
| chr3 | 144113304 | 144113354 |
| chr3 | 147134224 | 147134274 |
| chr3 | 150483743 | 150483793 |
| chr3 | 157001239 | 157001289 |
| chr3 | 178452418 | 178452468 |
| chr3 | 197299068 | 197299118 |
| chr4 | 13514780 | 13514830 |
| chr4 | 48210433 | 48210483 |
| chr4 | 140238915 | 140238965 |
| chr4 | 160380069 | 160380119 |
| chr4 | 160381966 | 160382016 |
| chr4 | 160382612 | 160382662 |
| chr11 | 67141506 | 67141556 |
| chr4 | 176203954 | 176204004 |
| chr4 | 185436887 | 185436937 |
| chr11 | 68019296 | 68019346 |
| chr5 | 43556883 | 43556933 |
| chr5 | 45936559 | 45936609 |
| chr5 | 53733108 | 53733158 |
| chr5 | 65270541 | 65270591 |
| chr5 | 80204338 | 80204388 |
| chr5 | 105270759 | 105270809 |
| chr5 | 117693901 | 117693951 |
| chr5 | 118376343 | 118376393 |
| chr5 | 134821989 | 134822039 |
| chr5 | 138525334 | 138525384 |
| chr5 | 151122935 | 151122985 |
| chr5 | 151888965 | 151889015 |
| chr11 | 76021087 | 76021137 |
| chr6 | 29969993 | 29970043 |
| chr6 | 32005671 | 32005721 |
| chr11 | 85292010 | 85292060 |
| chr11 | 85292396 | 85292446 |
| chr11 | 86208950 | 86209000 |
| chr6 | 43439030 | 43439080 |
| chr6 | 50825357 | 50825407 |
| chr6 | 58630865 | 58630915 |
| chr6 | 66803405 | 66803455 |
| chr6 | 74960358 | 74960408 |
| chr6 | 86486068 | 86486118 |
| chr6 | 87943856 | 87943906 |
| chr6 | 88442417 | 88442467 |
| chr6 | 105483836 | 105483886 |
| chr6 | 111384995 | 111385045 |
| chr6 | 118115817 | 118115867 |
| chr6 | 129389416 | 129389466 |
| chr6 | 140676975 | 140677025 |
| chr6 | 165067882 | 165067932 |
| chr6 | 165068088 | 165068138 |
| chr7 | 5519695 | 5519745 |
| chr7 | 76778003 | 76778053 |
| chr7 | 107050030 | 107050080 |
| chr7 | 111898941 | 111898991 |
| chr7 | 127882691 | 127882741 |
| chr7 | 128668004 | 128668054 |
| chr7 | 139221997 | 139222047 |
| chr7 | 154218861 | 154218911 |
| chr8 | 913745 | 913795 |
| chr8 | 11226468 | 11226518 |
| chr11 | 123811680 | 123811730 |
| chr8 | 52681243 | 52681293 |
| chr8 | 66578304 | 66578354 |
| chr8 | 68130536 | 68130586 |
| chr8 | 87392904 | 87392954 |
| chr8 | 100896929 | 100896979 |
| chr9 | 6939844 | 6939894 |
| chr9 | 33656621 | 33656671 |
| chr9 | 34030443 | 34030493 |
| chr9 | 37881172 | 37881222 |
| chr9 | 103017608 | 103017658 |
| chr9 | 109760450 | 109760500 |
| chr12 | 4669323 | 4669373 |
| chr12 | 6455793 | 6455843 |
| chr12 | 14398174 | 14398224 |
| chr12 | 24971168 | 24971218 |
| chr12 | 33068276 | 33068326 |
| chr12 | 37009491 | 37009541 |
| chr12 | 39034079 | 39034129 |
| chr12 | 47201642 | 47201692 |
| chr12 | 68438754 | 68438804 |
| chr10 | 29227381 | 29227431 |
| chr12 | 88400938 | 88400988 |
| chr12 | 107551344 | 107551394 |
| chr12 | 123891250 | 123891300 |
| chr13 | 44535964 | 44536014 |
| chr13 | 48133222 | 48133272 |
| chr13 | 79518935 | 79518985 |
| chr14 | 33612065 | 33612115 |
| chr14 | 37745688 | 37745738 |
| chr14 | 43059916 | 43059966 |
| chr14 | 62640744 | 62640794 |
| chr14 | 64150557 | 64150607 |
| chr14 | 68359326 | 68359376 |
| chr14 | 68933729 | 68933779 |
| chr14 | 82555168 | 82555218 |
| chr15 | 18664321 | 18664371 |
| chr10 | 66155260 | 66155310 |
| chr10 | 66155446 | 66155496 |
| chr10 | 66155772 | 66155822 |
| chr10 | 66155932 | 66155982 |
| chr10 | 68794843 | 68794893 |
| chr16 | 17567629 | 17567679 |
| chr16 | 31154953 | 31155003 |
| chr17 | 10160440 | 10160490 |
| chr17 | 16417159 | 16417209 |
| chr17 | 29009873 | 29009923 |
| chr17 | 33668878 | 33668928 |

Table 15EB. Repeat Category K.standard.SVA_D

| | | |
|---|---|---|
| chr17 | 37868936 | 37868986 |
| chr10 | 81959991 | 81960041 |
| chr10 | 81960244 | 81960294 |
| chr10 | 81960665 | 81960715 |
| chr10 | 81960944 | 81960994 |
| chr17 | 42004319 | 42004369 |
| chr17 | 43076361 | 43076411 |
| chr17 | 44804359 | 44804409 |
| chr17 | 45948984 | 45949034 |
| chr17 | 56627221 | 56627271 |
| chr17 | 56695603 | 56695653 |
| chr17 | 57947352 | 57947402 |
| chr17 | 60573292 | 60573342 |
| chr17 | 62007246 | 62007296 |
| chr17 | 64030006 | 64030056 |
| chr17 | 78921614 | 78921664 |
| chr17 | 79210018 | 79210068 |
| chr17 | 79212344 | 79212394 |
| chr10 | 92748136 | 92748186 |
| chr18 | 6818687 | 6818737 |
| chr18 | 9013011 | 9013061 |
| chr10 | 94398851 | 94398901 |
| chr18 | 12971495 | 12971545 |
| chr18 | 13089283 | 13089333 |
| chr18 | 14002100 | 14002150 |
| chr18 | 14373968 | 14374018 |
| chr18 | 31910823 | 31910873 |
| chr18 | 44859908 | 44859958 |
| chr18 | 53450752 | 53450802 |
| chr18 | 60425731 | 60425781 |
| chr18 | 62943894 | 62943944 |
| chr18 | 71840174 | 71840224 |
| chr18 | 72868729 | 72868779 |
| chr10 | 98954242 | 98954292 |
| chr10 | 99265275 | 99265325 |
| chr10 | 99576058 | 99576108 |
| chr19 | 1451859 | 1451909 |
| chr10 | 101700609 | 101700659 |
| chr19 | 5655128 | 5655178 |
| chr19 | 8710247 | 8710297 |
| chr19 | 11274598 | 11274648 |
| chr19 | 12438908 | 12438958 |
| chr19 | 15334075 | 15334125 |
| chr19 | 17978509 | 17978559 |
| chr19 | 17979127 | 17979177 |
| chr19 | 20036101 | 20036151 |
| chr19 | 20108932 | 20108982 |
| chr19 | 20123339 | 20123389 |
| chr19 | 20148629 | 20148679 |
| chr19 | 20506124 | 20506174 |
| chr19 | 21461933 | 21461983 |
| chr19 | 21487005 | 21487055 |
| chr19 | 21582097 | 21582147 |
| chr19 | 23147388 | 23147438 |
| chr19 | 23911260 | 23911310 |
| chr19 | 39314605 | 39314655 |
| chr19 | 39317296 | 39317346 |
| chr19 | 40104065 | 40104115 |
| chr19 | 41672182 | 41672232 |
| chr19 | 41672866 | 41672916 |
| chr19 | 41675457 | 41675507 |
| chr19 | 43547246 | 43547296 |
| chr19 | 43547461 | 43547511 |
| chr19 | 43547601 | 43547651 |
| chr19 | 43547800 | 43547850 |
| chr19 | 43548061 | 43548111 |
| chr19 | 43981578 | 43981628 |
| chr10 | 105032521 | 105032571 |
| chr19 | 46163266 | 46163316 |
| chr19 | 46165258 | 46165308 |
| chr19 | 46964552 | 46964602 |
| chr19 | 46964978 | 46965028 |
| chr19 | 46965165 | 46965215 |
| chr19 | 46965363 | 46965413 |
| chr19 | 47807284 | 47807334 |
| chr19 | 47807539 | 47807589 |
| chr19 | 49684062 | 49684112 |
| chr19 | 50840843 | 50840893 |
| chr19 | 50841209 | 50841259 |
| chr19 | 50841348 | 50841398 |
| chr19 | 50841695 | 50841745 |
| chr10 | 112192597 | 112192647 |
| chr19 | 57147868 | 57147918 |
| chr19 | 58417135 | 58417185 |
| chr19 | 58464391 | 58464441 |
| chr19 | 58465975 | 58466025 |
| chr19 | 58782007 | 58782057 |
| chr19 | 59410613 | 59410663 |
| chr19 | 63609643 | 63609693 |
| chr1 | 6075915 | 6075965 |
| chr1 | 6485917 | 6485967 |
| chr10 | 116211014 | 116211064 |
| chr1 | 7723387 | 7723437 |
| chr1 | 8269871 | 8269921 |
| chr1 | 8681661 | 8681711 |
| chr1 | 9797590 | 9797640 |
| chr10 | 6253552 | 6253602 |
| chr1 | 26528785 | 26528835 |
| chr10 | 120686024 | 120686074 |
| chr1 | 33476375 | 33476425 |
| chr1 | 34862119 | 34862169 |
| chr10 | 121220529 | 121220579 |
| chr1 | 38527309 | 38527359 |
| chr1 | 39114566 | 39114616 |
| chr1 | 39791216 | 39791266 |
| chr10 | 121640164 | 121640214 |
| chr1 | 44960171 | 44960221 |
| chr1 | 45376458 | 45376508 |
| chr1 | 48029195 | 48029245 |
| chr1 | 48314226 | 48314276 |
| chr1 | 50217707 | 50217757 |
| chr1 | 51548093 | 51548143 |
| chr1 | 52706478 | 52706528 |
| chr10 | 123628332 | 123628382 |
| chr1 | 76001630 | 76001680 |
| chr1 | 76002005 | 76002055 |
| chr1 | 76002671 | 76002721 |
| chr1 | 86789105 | 86789155 |
| chr1 | 86789450 | 86789500 |
| chr1 | 89730933 | 89730983 |
| chr1 | 96407451 | 96407501 |
| chr1 | 96408015 | 96408065 |
| chr1 | 96408356 | 96408406 |
| chr1 | 96700970 | 96701020 |
| chr1 | 98442195 | 98442245 |
| chr1 | 98442407 | 98442457 |
| chr1 | 106309087 | 106309137 |
| chr1 | 108058362 | 108058412 |
| chr1 | 108426146 | 108426196 |
| chr1 | 111074871 | 111074921 |
| chr1 | 111371416 | 111371466 |
| chr1 | 112481075 | 112481125 |
| chr1 | 114633908 | 114633958 |
| chr1 | 143416461 | 143416511 |
| chr1 | 146561408 | 146561458 |
| chr10 | 126780633 | 126780683 |
| chr1 | 148841397 | 148841447 |
| chr1 | 149385870 | 149385920 |
| chr1 | 149445795 | 149445845 |
| chr1 | 149662123 | 149662173 |
| chr1 | 151477705 | 151477755 |
| chr1 | 158469005 | 158469055 |
| chr1 | 158608730 | 158608780 |
| chr1 | 167576429 | 167576479 |
| chr1 | 173265792 | 173265842 |
| chr1 | 173606513 | 173606563 |
| chr1 | 176249568 | 176249618 |
| chr1 | 176628698 | 176628748 |
| chr1 | 179647472 | 179647522 |
| chr1 | 179951552 | 179951602 |
| chr1 | 194662399 | 194662449 |
| chr1 | 199448114 | 199448164 |
| chr1 | 199998056 | 199998106 |
| chr1 | 206381019 | 206381069 |
| chr1 | 209552410 | 209552460 |
| chr1 | 210008207 | 210008257 |
| chr1 | 210383109 | 210383159 |
| chr1 | 214754763 | 214754813 |
| chr1 | 217697475 | 217697525 |
| chr1 | 224294417 | 224294467 |
| chr1 | 224874141 | 224874191 |
| chr1 | 228300473 | 228300523 |
| chr1 | 229551038 | 229551088 |
| chr1 | 229552484 | 229552534 |
| chr1 | 234332911 | 234332961 |
| chr1 | 234873194 | 234873244 |
| chr1 | 243473979 | 243474029 |
| chr1 | 244141539 | 244141589 |
| chr1 | 245774636 | 245774686 |
| chr20 | 2996265 | 2996315 |
| chr20 | 3418043 | 3418093 |
| chr20 | 13735794 | 13735844 |
| chr20 | 17986966 | 17987016 |
| chr20 | 19654518 | 19654568 |
| chr20 | 24600970 | 24601020 |
| chr20 | 29422434 | 29422484 |
| chr20 | 31453944 | 31453994 |
| chr20 | 32489817 | 32489867 |
| chr20 | 32841002 | 32841052 |
| chr20 | 33002555 | 33002605 |
| chr20 | 33005345 | 33005395 |
| chr20 | 33779746 | 33779796 |
| chr20 | 36292374 | 36292424 |
| chr20 | 36325661 | 36325711 |
| chr20 | 36438601 | 36438651 |
| chr20 | 36441351 | 36441401 |
| chr20 | 37694101 | 37694151 |
| chr20 | 37694043 | 37694093 |
| chr20 | 37776992 | 37777042 |
| chr20 | 40870445 | 40870495 |
| chr20 | 42921658 | 42921708 |
| chr20 | 44960382 | 44960432 |
| chr20 | 44961126 | 44961176 |
| chr20 | 46841293 | 46841343 |
| chr20 | 48238876 | 48238926 |
| chr20 | 57674894 | 57674944 |
| chr20 | 60915301 | 60915351 |
| chr10 | 134702818 | 134702868 |
| chr21 | 28857115 | 28857165 |
| chr21 | 39448555 | 39448605 |
| chr21 | 46060422 | 46060472 |
| chr10 | 8914307 | 8914357 |
| chr22 | 16899855 | 16899905 |
| chr22 | 17263086 | 17263136 |
| chr22 | 17445438 | 17445488 |
| chr22 | 22227124 | 22227174 |
| chr22 | 22786667 | 22786717 |
| chr22 | 23039036 | 23039086 |
| chr22 | 23999369 | 23999419 |
| chr22 | 27275106 | 27275156 |
| chr22 | 28364660 | 28364710 |
| chr22 | 28580093 | 28580143 |
| chr22 | 33347827 | 33347877 |
| chr22 | 33404992 | 33405042 |
| chr22 | 34957881 | 34957931 |
| chr22 | 40271217 | 40271267 |
| chr22 | 40272363 | 40272413 |
| chr22 | 41378973 | 41379023 |
| chr11 | 3383795 | 3383845 |
| chrX | 6987354 | 6987404 |
| chrX | 6989970 | 6990020 |
| chrX | 12344840 | 12344890 |
| chr11 | 4123633 | 4123683 |
| chrX | 16345668 | 16345718 |
| chrX | 16349210 | 16349260 |
| chr11 | 5920275 | 5920325 |
| chrX | 28714872 | 28714922 |
| chrX | 36598374 | 36598424 |
| chrX | 40102942 | 40102992 |
| chrX | 40501423 | 40501473 |
| chrX | 41177142 | 41177192 |
| chrX | 41175076 | 41175126 |
| chrX | 43947517 | 43947567 |
| chrX | 45302502 | 45302552 |
| chrX | 47019969 | 47020019 |
| chrX | 47126800 | 47126850 |
| chrX | 51824554 | 51824604 |
| chrX | 54741759 | 54741809 |
| chrX | 68459317 | 68459367 |
| chrX | 68767264 | 68767314 |
| chrX | 69429912 | 69429962 |
| chrX | 72254796 | 72254846 |
| chrX | 72654158 | 72654208 |
| chrX | 75367287 | 75367337 |
| chrX | 75944307 | 75944357 |
| chrX | 76278215 | 76278265 |
| chrX | 78409296 | 78409346 |
| chrX | 78723449 | 78723499 |
| chrX | 94078171 | 94078221 |
| chrX | 101132432 | 101132482 |
| chrX | 117251510 | 117251560 |
| chrX | 121960814 | 121960864 |
| chrX | 124480096 | 124480146 |
| chrX | 129166861 | 129166911 |
| chrX | 132799266 | 132799316 |
| chrX | 134789452 | 134789502 |
| chrY | 8489041 | 8489091 |
| chrY | 13999269 | 13999319 |
| chr2 | 3204573 | 3204623 |
| chr2 | 4979181 | 4979231 |
| chr2 | 7499488 | 7499538 |
| chr2 | 11066028 | 11066078 |
| chr2 | 15702967 | 15703017 |
| chr2 | 15775102 | 15775152 |
| chr2 | 15841628 | 15841678 |
| chr2 | 25830525 | 25830575 |
| chr2 | 25919797 | 25919847 |
| chr2 | 26569299 | 26569349 |
| chr2 | 27738824 | 27738874 |
| chr2 | 32955231 | 32955281 |
| chr2 | 38135630 | 38135680 |
| chr11 | 19847158 | 19847208 |
| chr2 | 39830910 | 39830960 |
| chr2 | 42196472 | 42196522 |
| chr2 | 42217298 | 42217348 |
| chr2 | 43265845 | 43265895 |
| chr2 | 43880430 | 43880480 |
| chr2 | 44270329 | 44270379 |
| chr11 | 20849978 | 20850028 |
| chr2 | 47636478 | 47636528 |
| chr2 | 48815338 | 48815388 |
| chr2 | 51031762 | 51031812 |
| chr2 | 54298864 | 54298914 |
| chr2 | 63925042 | 63925092 |
| chr2 | 64011757 | 64011807 |
| chr2 | 64242968 | 64243018 |
| chr2 | 65803506 | 65803556 |
| chr2 | 74263993 | 74264043 |
| chr2 | 75927671 | 75927721 |
| chr2 | 95393385 | 95393435 |
| chr2 | 105488366 | 105488416 |
| chr2 | 113949003 | 113949053 |
| chr2 | 128203487 | 128203537 |
| chr2 | 133188017 | 133188067 |
| chr2 | 135032003 | 135032053 |
| chr2 | 135936087 | 135936137 |
| chr2 | 136675380 | 136675430 |
| chr11 | 33971889 | 33971939 |
| chr2 | 144889273 | 144889323 |
| chr2 | 144959683 | 144959733 |
| chr2 | 152935240 | 152935290 |
| chr2 | 153218611 | 153218661 |
| chr2 | 157351779 | 157351829 |
| chr2 | 157511773 | 157511823 |
| chr2 | 161499322 | 161499372 |
| chr2 | 170385154 | 170385204 |
| chr2 | 170563960 | 170564010 |
| chr2 | 170565362 | 170565412 |
| chr2 | 170635216 | 170635266 |
| chr2 | 171208056 | 171208106 |
| chr2 | 172927182 | 172927232 |
| chr2 | 172960183 | 172960233 |
| chr2 | 173604336 | 173604386 |
| chr2 | 178913322 | 178913372 |
| chr2 | 191499257 | 191499307 |
| chr2 | 192808333 | 192808383 |
| chr2 | 198764906 | 198764956 |
| chr2 | 202306974 | 202307024 |
| chr2 | 202630818 | 202630868 |
| chr2 | 203601228 | 203601278 |
| chr2 | 203783341 | 203783391 |
| chr2 | 204439048 | 204439098 |
| chr2 | 207240404 | 207240454 |
| chr2 | 207425287 | 207425337 |
| chr2 | 222958714 | 222958764 |
| chr2 | 223965719 | 223965769 |
| chr2 | 223966940 | 223966990 |
| chr2 | 230809338 | 230809388 |
| chr2 | 233974469 | 233974519 |
| chr2 | 240582472 | 240582522 |
| chr3 | 5130570 | 5130620 |
| chr3 | 5300631 | 5300681 |
| chr3 | 8457932 | 8457982 |
| chr3 | 9575852 | 9575902 |
| chr11 | 46884329 | 46884379 |
| chr3 | 10087134 | 10087184 |
| chr11 | 47026052 | 47026102 |
| chr3 | 12865423 | 12865473 |
| chr3 | 15077438 | 15077488 |
| chr3 | 15178450 | 15178500 |
| chr3 | 15758154 | 15758204 |
| chr3 | 16997646 | 16997696 |
| chr3 | 20884603 | 20884653 |
| chr3 | 23889024 | 23889074 |
| chr3 | 32182170 | 32182220 |
| chr3 | 39229007 | 39229057 |
| chr3 | 41307227 | 41307277 |
| chr3 | 44824130 | 44824180 |
| chr3 | 47955138 | 47955188 |
| chr3 | 50082426 | 50082476 |
| chr3 | 50309759 | 50309809 |
| chr11 | 55687513 | 55687563 |
| chr3 | 51304612 | 51304662 |
| chr3 | 51850498 | 51850548 |
| chr3 | 58233798 | 58233848 |
| chr10 | 12566958 | 12567008 |
| chr10 | 12569773 | 12569823 |
| chr3 | 61378741 | 61378791 |
| chr3 | 61613918 | 61613968 |
| chr3 | 61616094 | 61616144 |
| chr11 | 57260710 | 57260760 |
| chr3 | 71898403 | 71898453 |
| chr3 | 82553872 | 82553922 |
| chr3 | 96783608 | 96783658 |
| chr11 | 59127738 | 59127788 |
| chr11 | 59243434 | 59243484 |
| chr3 | 101519040 | 101519090 |
| chr3 | 104295845 | 104295895 |
| chr3 | 104571369 | 104571419 |
| chr3 | 107115684 | 107115734 |
| chr11 | 59744942 | 59744992 |
| chr3 | 109065331 | 109065381 |
| chr3 | 110164908 | 110164958 |
| chr3 | 113597058 | 113597108 |
| chr3 | 114087325 | 114087375 |
| chr3 | 114888903 | 114888953 |
| chr3 | 118091316 | 118091366 |
| chr3 | 119759055 | 119759105 |
| chr3 | 121454943 | 121454993 |
| chr3 | 123242235 | 123242285 |
| chr3 | 123821259 | 123821309 |
| chr3 | 125791162 | 125791212 |
| chr11 | 60710165 | 60710215 |
| chr3 | 130380868 | 130380918 |
| chr3 | 130404254 | 130404304 |
| chr3 | 132991237 | 132991287 |
| chr3 | 137764369 | 137764419 |
| chr3 | 138239288 | 138239338 |
| chr3 | 144127650 | 144127700 |
| chr3 | 146521762 | 146521812 |
| chr3 | 150420105 | 150420155 |
| chr3 | 151861016 | 151861066 |
| chr3 | 161218343 | 161218393 |
| chr3 | 176171693 | 176171743 |
| chr3 | 178931306 | 178931356 |
| chr3 | 180767784 | 180767834 |
| chr3 | 184645997 | 184646047 |
| chr3 | 186043662 | 186043712 |
| chr3 | 187851359 | 187851409 |
| chr3 | 187911898 | 187911948 |
| chr3 | 187924312 | 187924362 |
| chr3 | 191512025 | 191512075 |
| chr3 | 197597571 | 197597621 |
| chr4 | 378714 | 378764 |
| chr4 | 535300 | 535350 |
| chr4 | 961158 | 961208 |
| chr11 | 64244542 | 64244592 |
| chr4 | 16146889 | 16146939 |
| chr4 | 37566942 | 37566992 |
| chr4 | 38705577 | 38705627 |
| chr4 | 38707069 | 38707119 |
| chr4 | 38705526 | 38705576 |
| chr4 | 40040961 | 40041011 |
| chr4 | 40047464 | 40047514 |
| chr4 | 54017639 | 54017689 |
| chr4 | 54021168 | 54021218 |
| chr4 | 54038863 | 54038913 |
| chr4 | 54813739 | 54813789 |
| chr4 | 55108506 | 55108556 |
| chr4 | 57476050 | 57476100 |
| chr4 | 57954046 | 57954096 |
| chr4 | 58007002 | 58007052 |
| chr11 | 65305886 | 65305936 |
| chr4 | 70942426 | 70942476 |
| chr4 | 70944355 | 70944405 |
| chr4 | 70942485 | 70942535 |
| chr4 | 79364394 | 79364444 |
| chr4 | 84566567 | 84566617 |
| chr4 | 85324228 | 85324278 |
| chr4 | 95536618 | 95536668 |
| chr4 | 114339366 | 114339416 |
| chr4 | 119916655 | 119916705 |
| chr4 | 148857810 | 148857860 |
| chr4 | 152714116 | 152714166 |
| chr4 | 152754814 | 152754864 |
| chr4 | 152755106 | 152755156 |
| chr4 | 153024266 | 153024316 |
| chr4 | 154366316 | 154366366 |
| chr11 | 67021005 | 67021055 |
| chr4 | 166246269 | 166246319 |
| chr4 | 166520004 | 166520054 |
| chr4 | 171186399 | 171186449 |
| chr4 | 175912782 | 175912832 |
| chr4 | 176308166 | 176308216 |
| chr4 | 184658760 | 184658810 |
| chr4 | 186858654 | 186858704 |
| chr4 | 186858885 | 186858935 |
| chr4 | 189274453 | 189274503 |
| chr5 | 1638927 | 1638977 |
| chr5 | 16330731 | 16330781 |
| chr11 | 69869284 | 69869334 |
| chr5 | 37059846 | 37059896 |
| chr5 | 37610192 | 37610242 |
| chr5 | 39319088 | 39319138 |
| chr5 | 39379033 | 39379083 |
| chr5 | 43024757 | 43024807 |
| chr5 | 43134762 | 43134812 |
| chr5 | 43567004 | 43567054 |
| chr5 | 43624754 | 43624804 |
| chr5 | 53692253 | 53692303 |
| chr5 | 55009303 | 55009353 |
| chr5 | 55328011 | 55328061 |
| chr5 | 55351451 | 55351501 |
| chr5 | 59743627 | 59743677 |
| chr5 | 61826334 | 61826384 |
| chr5 | 62394801 | 62394851 |
| chr5 | 65289682 | 65289732 |
| chr5 | 67636126 | 67636176 |
| chr5 | 68376198 | 68376248 |
| chr5 | 70906132 | 70906182 |
| chr5 | 70932735 | 70932785 |
| chr11 | 70606443 | 70606493 |
| chr5 | 74649079 | 74649129 |
| chr5 | 74652201 | 74652251 |
| chr5 | 75716644 | 75716694 |
| chr5 | 76194250 | 76194300 |
| chr5 | 76365845 | 76365895 |
| chr5 | 77771976 | 77772026 |
| chr5 | 79682758 | 79682808 |
| chr5 | 80323798 | 80323848 |
| chr5 | 83692597 | 83692647 |
| chr5 | 87994980 | 87995030 |
| chr5 | 107972342 | 107972392 |
| chr5 | 108403271 | 108403321 |
| chr5 | 112367724 | 112367774 |
| chr5 | 117012722 | 117012772 |
| chr5 | 118534935 | 118534985 |
| chr5 | 134294759 | 134294809 |
| chr5 | 138188367 | 138188417 |
| chr5 | 138190326 | 138190376 |
| chr5 | 138223846 | 138223896 |
| chr5 | 138468282 | 138468332 |
| chr5 | 138637318 | 138637368 |
| chr5 | 139836744 | 139836794 |
| chr11 | 73335602 | 73335652 |
| chr11 | 73375081 | 73375131 |
| chr11 | 73567595 | 73567645 |
| chr5 | 149079492 | 149079542 |
| chr5 | 152445451 | 152445501 |
| chr5 | 152706035 | 152706085 |
| chr11 | 74849112 | 74849162 |
| chr11 | 75316270 | 75316320 |
| chr5 | 175660174 | 175660224 |
| chr11 | 76371315 | 76371365 |
| chr5 | 179179616 | 179179666 |
| chr5 | 180308543 | 180308593 |
| chr6 | 5781917 | 5781967 |
| chr6 | 15525144 | 15525194 |
| chr6 | 15835798 | 15835848 |
| chr11 | 78629649 | 78629699 |
| chr6 | 17842796 | 17842846 |
| chr6 | 20046727 | 20046777 |
| chr6 | 21906600 | 21906650 |
| chr6 | 22943720 | 22943770 |
| chr6 | 27901757 | 27901807 |
| chr6 | 27904250 | 27904300 |
| chr11 | 82569779 | 82569829 |
| chr6 | 30007332 | 30007382 |
| chr11 | 82755217 | 82755267 |
| chr6 | 31068038 | 31068088 |
| chr6 | 31529046 | 31529096 |
| chr6 | 32047682 | 32047732 |
| chr6 | 32425122 | 32425172 |
| chr6 | 33075498 | 33075548 |
| chr6 | 33846472 | 33846522 |
| chr11 | 85546999 | 85547049 |
| chr6 | 41630683 | 41630733 |
| chr6 | 42481945 | 42481995 |
| chr11 | 86168516 | 86168566 |
| chr6 | 48384900 | 48384950 |
| chr6 | 52568954 | 52569004 |
| chr6 | 53218016 | 53218066 |
| chr6 | 55822642 | 55822692 |
| chr6 | 56735593 | 56735643 |
| chr6 | 71971793 | 71971843 |
| chr6 | 74306245 | 74306295 |
| chr6 | 74557303 | 74557353 |
| chr6 | 74620521 | 74620571 |
| chr11 | 90485942 | 90485992 |
| chr6 | 76148174 | 76148224 |
| chr6 | 84949859 | 84949909 |
| chr6 | 88457425 | 88457475 |
| chr6 | 100144530 | 100144580 |
| chr6 | 106348914 | 106348964 |
| chr6 | 108169879 | 108169929 |
| chr6 | 108301147 | 108301197 |
| chr6 | 108320119 | 108320169 |
| chr6 | 112415165 | 112415215 |
| chr6 | 112895991 | 112896041 |
| chr11 | 94903240 | 94903290 |
| chr6 | 135666863 | 135666913 |
| chr6 | 144736050 | 144736100 |
| chr6 | 148481686 | 148481736 |
| chr6 | 150138187 | 150138237 |
| chr6 | 151801723 | 151801773 |
| chr6 | 154616129 | 154616179 |
| chr6 | 155318861 | 155318911 |
| chr6 | 158640582 | 158640632 |
| chr11 | 105725765 | 105725815 |
| chr11 | 107320113 | 107320163 |
| chr7 | 2086044 | 2086094 |
| chr7 | 4174729 | 4174779 |
| chr7 | 6456810 | 6456860 |
| chr7 | 6759932 | 6759982 |
| chr7 | 16575222 | 16575272 |
| chr7 | 22828634 | 22828684 |
| chr7 | 25929316 | 25929366 |
| chr7 | 26043031 | 26043081 |
| chr7 | 28621555 | 28621605 |
| chr7 | 35977068 | 35977118 |
| chr7 | 47738995 | 47739045 |
| chr7 | 50268181 | 50268231 |
| chr7 | 55122036 | 55122086 |
| chr7 | 55213641 | 55213691 |
| chr7 | 55959316 | 55959366 |
| chr7 | 62746739 | 62746789 |
| chr7 | 63611692 | 63611742 |
| chr7 | 63682520 | 63682570 |
| chr7 | 63841399 | 63841449 |
| chr7 | 75195014 | 75195064 |
| chr7 | 75195590 | 75195640 |
| chr7 | 76776743 | 76776793 |
| chr7 | 87508754 | 87508804 |
| chr7 | 89986298 | 89986348 |
| chr7 | 98638080 | 98638130 |
| chr7 | 99098634 | 99098684 |
| chr7 | 99733707 | 99733757 |
| chr7 | 110809782 | 110809832 |
| chr7 | 111751609 | 111751659 |
| chr7 | 127771776 | 127771826 |
| chr7 | 132084922 | 132084972 |
| chr7 | 132277862 | 132277912 |
| chr7 | 135314001 | 135314051 |
| chr7 | 138529099 | 138529149 |
| chr7 | 146891648 | 146891698 |
| chr7 | 148369249 | 148369299 |
| chr7 | 148642197 | 148642247 |
| chr7 | 149594275 | 149594325 |
| chr8 | 9049806 | 9049856 |
| chr8 | 9260307 | 9260357 |
| chr8 | 9260370 | 9260420 |
| chr8 | 21579310 | 21579360 |
| chr11 | 124112011 | 124112061 |
| chr8 | 33574521 | 33574571 |
| chr8 | 34442947 | 34442997 |
| chr8 | 34445769 | 34445819 |
| chr8 | 37841030 | 37841080 |
| chr8 | 42814240 | 42814290 |
| chr8 | 43293109 | 43293159 |
| chr8 | 43561357 | 43561407 |
| chr8 | 57235757 | 57235807 |
| chr8 | 58034874 | 58034924 |
| chr8 | 59639792 | 59639842 |
| chr8 | 61512102 | 61512152 |
| chr8 | 64143646 | 64143696 |
| chr8 | 66623455 | 66623505 |
| chr8 | 67193684 | 67193734 |
| chr8 | 68423058 | 68423108 |
| chr8 | 87664553 | 87664603 |
| chr8 | 94007310 | 94007360 |
| chr8 | 101860209 | 101860259 |
| chr8 | 103898175 | 103898225 |
| chr8 | 122626152 | 122626202 |
| chr11 | 129254868 | 129254918 |
| chr8 | 124285606 | 124285656 |
| chr8 | 125401788 | 125401838 |
| chr8 | 129088391 | 129088441 |
| chr8 | 129088470 | 129088520 |
| chr8 | 131891567 | 131891617 |
| chr8 | 134725470 | 134725520 |
| chr8 | 142184759 | 142184809 |
| chr9 | 6612209 | 6612259 |
| chr9 | 16966537 | 16966587 |
| chr9 | 19423098 | 19423148 |
| chr9 | 29180047 | 29180097 |
| chr9 | 70879296 | 70879346 |
| chr9 | 70923881 | 70923931 |
| chr9 | 75026755 | 75026805 |
| chr9 | 75133827 | 75133877 |
| chr9 | 83645073 | 83645123 |
| chr9 | 84383507 | 84383557 |
| chr9 | 85832215 | 85832265 |
| chr9 | 86318262 | 86318312 |
| chr9 | 87064208 | 87064258 |
| chr9 | 90331814 | 90331864 |
| chr9 | 90332049 | 90332099 |
| chr9 | 90933510 | 90933560 |
| chr9 | 95938036 | 95938086 |
| chr9 | 98209195 | 98209245 |
| chr9 | 105413194 | 105413244 |
| chr9 | 108240280 | 108240330 |
| chr9 | 114979963 | 114980013 |
| chr9 | 120989652 | 120989702 |
| chr9 | 123598178 | 123598228 |
| chr9 | 123920214 | 123920264 |
| chr9 | 128658826 | 128658876 |
| chr12 | 7242914 | 7242964 |
| chr12 | 7635369 | 7635419 |
| chr12 | 8668093 | 8668143 |
| chr12 | 12459818 | 12459868 |
| chr12 | 12609163 | 12609213 |
| chr12 | 19384479 | 19384529 |
| chr12 | 28246451 | 28246501 |
| chr12 | 32608405 | 32608455 |
| chr12 | 47181585 | 47181635 |
| chr12 | 47524001 | 47524051 |
| chr12 | 47596738 | 47596788 |
| chr12 | 47963728 | 47963778 |
| chr12 | 47963802 | 47963852 |
| chr12 | 50326226 | 50326276 |
| chr12 | 51090801 | 51090851 |
| chr12 | 53168476 | 53168526 |
| chr12 | 54160277 | 54160327 |
| chr12 | 55464978 | 55465028 |
| chr12 | 55470652 | 55470702 |
| chr12 | 56188739 | 56188789 |
| chr12 | 74620705 | 74620755 |
| chr12 | 79205675 | 79205725 |
| chr12 | 94132693 | 94132743 |
| chr12 | 94804907 | 94804957 |
| chr12 | 95055588 | 95055638 |
| chr12 | 95055532 | 95055582 |
| chr12 | 99192185 | 99192235 |
| chr12 | 109072525 | 109072575 |
| chr12 | 109075119 | 109075169 |
| chr12 | 109801604 | 109801654 |
| chr12 | 110573855 | 110573905 |
| chr12 | 118210960 | 118211010 |
| chr12 | 121632499 | 121632549 |
| chr12 | 122937497 | 122937547 |
| chr12 | 129835954 | 129836004 |
| chr10 | 35698339 | 35698389 |
| chr13 | 21232371 | 21232421 |
| chr13 | 23344692 | 23344742 |
| chr13 | 23853347 | 23853397 |
| chr13 | 23886147 | 23886197 |
| chr13 | 25096987 | 25097037 |
| chr13 | 25178527 | 25178577 |
| chr13 | 28641011 | 28641061 |
| chr13 | 29213112 | 29213162 |
| chr13 | 30373477 | 30373527 |
| chr13 | 31087728 | 31087778 |
| chr13 | 35651609 | 35651659 |
| chr13 | 40253388 | 40253438 |
| chr13 | 43053772 | 43053822 |
| chr13 | 50451281 | 50451331 |
| chr10 | 42625880 | 42625930 |
| chr13 | 79474134 | 79474184 |
| chr13 | 86327920 | 86327970 |
| chr13 | 94042622 | 94042672 |
| chr13 | 97593377 | 97593427 |
| chr13 | 106132342 | 106132392 |
| chr13 | 108957655 | 108957705 |
| chr13 | 112443952 | 112444002 |
| chr10 | 45111597 | 45111647 |
| chr14 | 18670218 | 18670268 |
| chr14 | 18963225 | 18963275 |
| chr10 | 45290795 | 45290845 |
| chr14 | 29897533 | 29897583 |
| chr14 | 32539370 | 32539420 |
| chr14 | 34285635 | 34285685 |
| chr14 | 34285988 | 34286038 |
| chr14 | 34437910 | 34437960 |
| chr14 | 36015784 | 36015834 |
| chr14 | 40131951 | 40132001 |
| chr14 | 40134862 | 40134912 |
| chr14 | 40135220 | 40135270 |
| chr14 | 53913324 | 53913374 |
| chr14 | 57514566 | 57514616 |
| chr14 | 61621604 | 61621654 |
| chr14 | 62495720 | 62495770 |
| chr14 | 65002647 | 65002697 |
| chr14 | 70693616 | 70693666 |
| chr14 | 71642570 | 71642620 |
| chr10 | 52497811 | 52497861 |
| chr10 | 52497995 | 52498045 |
| chr14 | 74382855 | 74382905 |
| chr14 | 75189882 | 75189932 |
| chr14 | 75277927 | 75277977 |
| chr14 | 76450964 | 76451014 |
| chr14 | 78741830 | 78741880 |
| chr14 | 81271688 | 81271738 |
| chr14 | 83809998 | 83810048 |
| chr14 | 84287070 | 84287120 |
| chr14 | 90014834 | 90014884 |
| chr14 | 100629249 | 100629299 |
| chr14 | 101134995 | 101135045 |
| chr14 | 103518394 | 103518444 |
| chr15 | 24812598 | 24812648 |
| chr15 | 32954594 | 32954644 |
| chr15 | 41176502 | 41176552 |
| chr15 | 43302398 | 43302448 |
| chr15 | 47559523 | 47559573 |
| chr10 | 64915612 | 64915662 |
| chr10 | 64916068 | 64916118 |
| chr10 | 64916861 | 64916911 |
| chr10 | 64917206 | 64917256 |
| chr15 | 60341617 | 60341667 |
| chr15 | 60699306 | 60699356 |
| chr15 | 66141657 | 66141707 |
| chr15 | 66145040 | 66145090 |
| chr15 | 66866763 | 66866813 |
| chr15 | 66869293 | 66869343 |
| chr15 | 66973811 | 66973861 |
| chr10 | 68794384 | 68794434 |
| chr10 | 68795183 | 68795233 |
| chr15 | 70816629 | 70816679 |
| chr15 | 72738079 | 72738129 |
| chr15 | 82211845 | 82211895 |
| chr15 | 82945019 | 82945069 |
| chr15 | 87568839 | 87568889 |
| chr15 | 88250004 | 88250054 |
| chr15 | 90880648 | 90880698 |
| chr15 | 91042469 | 91042519 |
| chr15 | 99050468 | 99050518 |
| chr16 | 2210094 | 2210144 |
| chr16 | 2211173 | 2211223 |
| chr16 | 2390927 | 2390977 |
| chr16 | 3321835 | 3321885 |
| chr16 | 7952462 | 7952512 |
| chr16 | 8795301 | 8795351 |
| chr16 | 11396349 | 11396399 |
| chr10 | 71512902 | 71512952 |
| chr10 | 71513053 | 71513103 |
| chr16 | 14593842 | 14593892 |
| chr10 | 71734796 | 71734846 |
| chr16 | 31150915 | 31150965 |
| chr16 | 31279128 | 31279178 |
| chr16 | 31415480 | 31415530 |
| chr16 | 31883300 | 31883350 |
| chr16 | 46469163 | 46469213 |
| chr16 | 46814287 | 46814337 |
| chr16 | 48762926 | 48762976 |
| chr16 | 49023177 | 49023227 |
| chr10 | 72975383 | 72975433 |
| chr10 | 72975897 | 72975947 |
| chr10 | 72976020 | 72976070 |
| chr10 | 72981158 | 72981208 |
| chr10 | 72981303 | 72981353 |
| chr16 | 53350948 | 53350998 |
| chr16 | 53510930 | 53510980 |
| chr16 | 57011286 | 57011336 |
| chr16 | 58207131 | 58207181 |
| chr16 | 58656372 | 58656422 |
| chr16 | 67974562 | 67974612 |
| chr16 | 69091625 | 69091675 |
| chr16 | 69859654 | 69859704 |
| chr16 | 71398847 | 71398897 |
| chr16 | 80058285 | 80058335 |
| chr16 | 89272919 | 89272969 |
| chr17 | 1800608 | 1800658 |
| chr17 | 2616089 | 2616139 |
| chr17 | 3221653 | 3221703 |
| chr17 | 3482778 | 3482828 |
| chr17 | 5246313 | 5246363 |
| chr17 | 5825875 | 5825925 |
| chr17 | 8655083 | 8655133 |
| chr17 | 16084032 | 16084082 |
| chr17 | 16198066 | 16198116 |
| chr17 | 17547553 | 17547603 |
| chr17 | 18017576 | 18017626 |
| chr10 | 79574109 | 79574159 |
| chr10 | 79574395 | 79574445 |
| chr17 | 27077855 | 27077905 |
| chr17 | 28005198 | 28005248 |
| chr17 | 28996952 | 28997002 |
| chr17 | 28995257 | 28995307 |
| chr17 | 29209120 | 29209170 |
| chr17 | 37161394 | 37161444 |
| chr17 | 37462476 | 37462526 |

Table 15EC. Repeat Category K.standard.SVA_E

| | | |
|---|---|---|
| chr17 | 76370870 | 76370920 |
| chr10 | 98366801 | 98366851 |
| chr19 | 21694870 | 21694920 |
| chr19 | 23108961 | 23109011 |
| chr19 | 58004902 | 58004952 |
| chr19 | 58115312 | 58115362 |
| chr19 | 58384220 | 58384270 |
| chr1 | 7723985 | 7724035 |
| chr1 | 24509715 | 24509765 |
| chr1 | 24512635 | 24512685 |
| chr1 | 26994783 | 26994833 |
| chr1 | 32940987 | 32941037 |
| chr1 | 32941385 | 32941435 |
| chr1 | 32942019 | 32942069 |
| chr1 | 32944060 | 32944110 |
| chr1 | 42629117 | 42629167 |
| chr1 | 45102917 | 45102967 |
| chr1 | 48220000 | 48220050 |
| chr1 | 77759401 | 77759451 |
| chr1 | 148790802 | 148790852 |
| chr1 | 153745730 | 153745780 |
| chr1 | 209527408 | 209527458 |
| chr1 | 231488680 | 231488730 |
| chr20 | 2798081 | 2798131 |
| chr20 | 4007499 | 4007549 |
| chr20 | 53551561 | 53551611 |
| chr21 | 43092298 | 43092348 |
| chr22 | 29746663 | 29746713 |
| chr22 | 29748744 | 29748794 |
| chrX | 52766633 | 52766683 |
| chrX | 66412501 | 66412551 |
| chrX | 66415982 | 66416032 |
| chrX | 70266731 | 70266781 |
| chr11 | 8963146 | 8963196 |
| chrX | 90848034 | 90848084 |
| chr2 | 117882 | 117932 |
| chr2 | 15382130 | 15382180 |
| chr2 | 27756436 | 27756486 |
| chr2 | 32648272 | 32648322 |
| chr2 | 44791739 | 44791789 |
| chr2 | 129270462 | 129270512 |
| chr2 | 133191550 | 133191600 |
| chr2 | 192466848 | 192466898 |
| chr2 | 201752061 | 201752111 |
| chr2 | 202349139 | 202349189 |
| chr2 | 215903950 | 215904000 |
| chr2 | 217295015 | 217295065 |
| chr3 | 38085537 | 38085587 |
| chr3 | 131906916 | 131906966 |
| chr3 | 182965395 | 182965445 |
| chr4 | 106490235 | 106490285 |
| chr4 | 185354845 | 185354895 |
| chr5 | 40845960 | 40846010 |
| chr5 | 137098703 | 137098753 |
| chr6 | 4982114 | 4982164 |
| chr6 | 21960749 | 21960799 |
| chr6 | 51892806 | 51892856 |
| chr6 | 56804762 | 56804812 |
| chr6 | 131446015 | 131446065 |
| chr6 | 131445396 | 131445446 |
| chr7 | 2319330 | 2319380 |
| chr7 | 20451686 | 20451736 |
| chr7 | 32169759 | 32169809 |
| chr7 | 151676285 | 151676335 |
| chr8 | 30163171 | 30163221 |
| chr8 | 43053908 | 43053958 |
| chr8 | 49111125 | 49111175 |
| chr8 | 99998632 | 99998682 |
| chr8 | 117649872 | 117649922 |
| chr8 | 128598924 | 128598974 |
| chr11 | 130492741 | 130492791 |
| chr9 | 132101873 | 132101923 |
| chr9 | 132102501 | 132102551 |
| chr9 | 132103362 | 132103412 |
| chr9 | 132104232 | 132104282 |
| chr12 | 8496022 | 8496072 |
| chr12 | 8671060 | 8671110 |
| chr12 | 9392903 | 9392953 |
| chr12 | 68881105 | 68881155 |
| chr12 | 88764115 | 88764165 |
| chr12 | 94738767 | 94738817 |
| chr10 | 32264631 | 32264681 |
| chr10 | 43202584 | 43202634 |
| chr14 | 21098152 | 21098202 |
| chr14 | 63350373 | 63350423 |
| chr14 | 63353560 | 63353610 |
| chr10 | 64053500 | 64053550 |
| chr10 | 2803222 | 2803272 |
| chr10 | 71098249 | 71098299 |
| chr10 | 71098476 | 71098526 |
| chr16 | 31153208 | 31153258 |
| chr16 | 69861995 | 69862045 |
| chr16 | 72306832 | 72306882 |
| chr17 | 25683858 | 25683908 |
| chr17 | 29453937 | 29453987 |
| chr17 | 31393734 | 31393784 |
| chr17 | 31396394 | 31396444 |

Table 15ED. Repeat Category K.standard.SVA_F

| | | |
|---|---|---|
| chr17 | 42734324 | 42734374 |
| chr17 | 59104693 | 59104743 |
| chr17 | 79274921 | 79274971 |
| chr17 | 79277553 | 79277603 |
| chr18 | 45357576 | 45357626 |
| chr18 | 45577496 | 45577546 |
| chr18 | 50122968 | 50123018 |
| chr10 | 101516416 | 101516466 |
| chr10 | 101519286 | 101519336 |
| chr19 | 12594362 | 12594412 |
| chr19 | 13674463 | 13674513 |
| chr19 | 21841739 | 21841789 |
| chr19 | 22083508 | 22083558 |
| chr19 | 39261074 | 39261124 |
| chr19 | 39263427 | 39263477 |
| chr10 | 104552012 | 104552062 |
| chr19 | 45307583 | 45307633 |
| chr19 | 45307871 | 45307921 |
| chr10 | 106209953 | 106210003 |
| chr10 | 6101851 | 6101901 |
| chr1 | 20820932 | 20820982 |
| chr1 | 24478767 | 24478817 |
| chr1 | 26750546 | 26750596 |
| chr1 | 27786309 | 27786359 |
| chr10 | 121147198 | 121147248 |
| chr1 | 54068147 | 54068197 |
| chr1 | 54396246 | 54396296 |
| chr1 | 108564251 | 108564301 |
| chr1 | 111450444 | 111450494 |
| chr1 | 111641250 | 111641300 |
| chr1 | 112616833 | 112616883 |
| chr1 | 112740854 | 112740904 |
| chr1 | 112913061 | 112913111 |
| chr1 | 117317734 | 117317784 |
| chr1 | 117320217 | 117320267 |
| chr1 | 147916626 | 147916676 |
| chr1 | 147918894 | 147918944 |
| chr1 | 152887053 | 152887103 |
| chr1 | 171556599 | 171556649 |
| chr1 | 212100487 | 212100537 |
| chr1 | 238620763 | 238620813 |
| chr20 | 16204293 | 16204343 |
| chr20 | 31510455 | 31510505 |
| chr20 | 34455925 | 34455975 |
| chr21 | 17587571 | 17587621 |
| chr21 | 45543355 | 45543405 |
| chr22 | 25494761 | 25494811 |
| chr22 | 30316215 | 30316265 |
| chrX | 24089807 | 24089857 |
| chrX | 41365260 | 41365310 |
| chrX | 51823646 | 51823696 |
| chrX | 61277107 | 61277157 |
| chrX | 70769609 | 70769659 |
| chrX | 74306383 | 74306433 |
| chrX | 99000336 | 99000386 |
| chrX | 113347469 | 113347519 |
| chrX | 145741311 | 145741361 |
| chrX | 152469870 | 152469920 |
| chr11 | 17037506 | 17037556 |
| chr2 | 41947661 | 41947711 |
| chr2 | 41950404 | 41950454 |
| chr2 | 42808785 | 42808835 |
| chr2 | 43498271 | 43498321 |
| chr2 | 43500315 | 43500365 |
| chr2 | 48756212 | 48756262 |
| chr2 | 48760079 | 48760129 |
| chr2 | 55976416 | 55976466 |
| chr2 | 64713834 | 64713884 |
| chr11 | 28415051 | 28415101 |
| chr2 | 96957427 | 96957477 |
| chr2 | 108965316 | 108965366 |
| chr2 | 120698151 | 120698201 |
| chr2 | 127971499 | 127971549 |
| chr2 | 187222385 | 187222435 |
| chr2 | 198989211 | 198989261 |
| chr2 | 198991529 | 198991579 |
| chr2 | 207467114 | 207467164 |
| chr2 | 223884739 | 223884789 |
| chr11 | 44650882 | 44650932 |
| chr2 | 234920756 | 234920806 |
| chr11 | 45725549 | 45725599 |
| chr3 | 10433363 | 10433413 |
| chr11 | 47082844 | 47082894 |
| chr3 | 11504319 | 11504369 |
| chr3 | 17722705 | 17722755 |
| chr3 | 42798928 | 42798978 |
| chr3 | 51244702 | 51244752 |
| chr3 | 57689952 | 57690002 |
| chr3 | 58834519 | 58834569 |
| chr3 | 67426687 | 67426737 |
| chr3 | 77134394 | 77134444 |
| chr3 | 99879137 | 99879187 |
| chr3 | 102478471 | 102478521 |
| chr3 | 107151613 | 107151663 |
| chr3 | 123203406 | 123203456 |
| chr3 | 123206645 | 123206695 |
| chr3 | 125409128 | 125409178 |
| chr3 | 129992950 | 129993000 |
| chr3 | 133511983 | 133512033 |
| chr3 | 134823890 | 134823940 |
| chr3 | 180330420 | 180330470 |
| chr4 | 1790346 | 1790396 |
| chr4 | 48282449 | 48282499 |
| chr4 | 53244584 | 53244634 |
| chr4 | 64787918 | 64787968 |
| chr4 | 147192912 | 147192962 |
| chr4 | 152159877 | 152159927 |
| chr4 | 159135764 | 159135814 |
| chr4 | 159136799 | 159136849 |
| chr4 | 161439611 | 161439661 |
| chr4 | 182562933 | 182562983 |
| chr5 | 34353384 | 34353434 |
| chr5 | 77174987 | 77175037 |
| chr5 | 81518850 | 81518900 |
| chr5 | 81521351 | 81521401 |
| chr5 | 108755363 | 108755413 |
| chr11 | 71940955 | 71941005 |
| chr5 | 141485397 | 141485447 |
| chr5 | 167266580 | 167266630 |
| chr5 | 168052697 | 168052747 |
| chr5 | 170555625 | 170555675 |
| chr5 | 170812871 | 170812921 |
| chr5 | 175711689 | 175711739 |
| chr5 | 176368109 | 176368159 |
| chr5 | 176368289 | 176368339 |
| chr5 | 178060017 | 178060067 |
| chr11 | 76534761 | 76534811 |
| chr11 | 76537673 | 76537723 |
| chr6 | 15296543 | 15296593 |
| chr6 | 27562392 | 27562442 |
| chr6 | 31925025 | 31925075 |
| chr6 | 32668245 | 32668295 |
| chr6 | 34411459 | 34411509 |
| chr6 | 35354218 | 35354268 |
| chr6 | 35355721 | 35355771 |
| chr6 | 35356410 | 35356460 |
| chr6 | 35357115 | 35357165 |
| chr6 | 38979060 | 38979110 |
| chr6 | 43596909 | 43596959 |
| chr6 | 47521404 | 47521454 |
| chr6 | 87716483 | 87716533 |
| chr11 | 93369556 | 93369606 |
| chr6 | 109412841 | 109412891 |
| chr6 | 109414914 | 109414964 |
| chr6 | 112875966 | 112876016 |
| chr11 | 94743675 | 94743725 |
| chr6 | 123152233 | 123152283 |
| chr6 | 158458359 | 158458409 |
| chr6 | 162668401 | 162668451 |
| chr7 | 820041 | 820091 |
| chr7 | 820866 | 820916 |
| chr7 | 2053522 | 2053572 |
| chr7 | 10250728 | 10250778 |
| chr7 | 24799709 | 24799759 |
| chr7 | 29742896 | 29742946 |
| chr7 | 51018528 | 51018578 |
| chr7 | 55030827 | 55030877 |
| chr7 | 99951893 | 99951943 |
| chr11 | 118917246 | 118917296 |
| chr10 | 17676024 | 17676074 |
| chr8 | 90802 | 90852 |
| chr8 | 5569478 | 5569528 |
| chr8 | 62716767 | 62716817 |
| chr8 | 65647631 | 65647681 |
| chr8 | 68274453 | 68274503 |
| chr8 | 124283125 | 124283175 |
| chr8 | 128287078 | 128287128 |
| chr8 | 129796003 | 129796053 |
| chr11 | 129500602 | 129500652 |
| chr9 | 6796826 | 6796876 |
| chr9 | 36606303 | 36606353 |
| chr9 | 70914608 | 70914658 |
| chr9 | 74135004 | 74135054 |
| chr9 | 94849984 | 94850034 |
| chr9 | 94850112 | 94850162 |
| chr9 | 94850295 | 94850345 |
| chr9 | 105400664 | 105400714 |
| chr9 | 105920431 | 105920481 |
| chr9 | 110658031 | 110658081 |
| chr9 | 118635803 | 118635853 |
| chr9 | 121196388 | 121196438 |
| chr9 | 123467552 | 123467602 |
| chr12 | 3075706 | 3075756 |
| chr10 | 21723109 | 21723159 |
| chr12 | 9995855 | 9995905 |
| chr12 | 9995804 | 9995854 |
| chr12 | 36496322 | 36496372 |
| chr12 | 54756803 | 54756853 |
| chr10 | 27590438 | 27590488 |
| chr12 | 64813502 | 64813552 |
| chr12 | 97551979 | 97552029 |
| chr12 | 103218909 | 103218959 |
| chr12 | 109715997 | 109716047 |
| chr12 | 112721510 | 112721560 |
| chr12 | 118769879 | 118769929 |
| chr12 | 120828334 | 120828384 |
| chr13 | 18150467 | 18150517 |
| chr13 | 19819222 | 19819272 |
| chr13 | 32911782 | 32911832 |
| chr13 | 32913879 | 32913929 |
| chr13 | 41337783 | 41337833 |
| chr13 | 43858943 | 43858993 |
| chr13 | 44887929 | 44887979 |
| chr13 | 71948442 | 71948492 |
| chr14 | 33497728 | 33497778 |
| chr14 | 43848168 | 43848218 |
| chr14 | 43850302 | 43850352 |
| chr14 | 91369269 | 91369319 |
| chr15 | 33663776 | 33663826 |
| chr15 | 37903549 | 37903599 |
| chr15 | 39392511 | 39392561 |
| chr15 | 73488837 | 73488887 |
| chr15 | 88126493 | 88126543 |
| chr15 | 88139945 | 88139995 |
| chr15 | 88142660 | 88142710 |
| chr16 | 1588642 | 1588692 |
| chr16 | 2853159 | 2853209 |
| chr10 | 71793315 | 71793365 |
| chr10 | 71793421 | 71793471 |
| chr16 | 30216836 | 30216886 |
| chr16 | 46509889 | 46509939 |
| chr16 | 56550325 | 56550375 |
| chr10 | 76438594 | 76438644 |
| chr10 | 76438918 | 76438968 |
| chr17 | 15742449 | 15742499 |
| chr17 | 18992705 | 18992755 |
| chr17 | 18993007 | 18993057 |
| chr17 | 19694535 | 19694585 |
| chr17 | 26929269 | 26929319 |
| chr17 | 36120972 | 36121022 |

Table 15EE. Repeat Category THE1B

| | | |
|---|---|---|
| chr17 | 42046940 | 42046990 |
| chr17 | 44573850 | 44573900 |
| chr17 | 47210829 | 47210879 |
| chr17 | 53924503 | 53924553 |
| chr17 | 55593418 | 55593468 |
| chr17 | 56249321 | 56249371 |
| chr17 | 58985856 | 58985906 |
| chr17 | 64629756 | 64629806 |
| chr17 | 67420693 | 67420743 |
| chr17 | 68156863 | 68156913 |
| chr17 | 68806705 | 68806755 |
| chr17 | 69052188 | 69052238 |
| chr17 | 71090845 | 71090895 |
| chr17 | 71176123 | 71176173 |
| chr17 | 71743866 | 71743916 |
| chr17 | 73016291 | 73016341 |
| chr17 | 75446350 | 75446400 |
| chr10 | 90222619 | 90222669 |
| chr17 | 77252911 | 77252961 |
| chr17 | 77260496 | 77260546 |
| chr17 | 78442231 | 78442281 |
| chr17 | 79323136 | 79323186 |
| chr17 | 80036141 | 80036191 |
| chr17 | 81671328 | 81671378 |
| chr18 | 829547 | 829597 |
| chr18 | 829394 | 829444 |
| chr10 | 94044869 | 94044919 |
| chr18 | 10689011 | 10689061 |
| chr18 | 19271431 | 19271481 |
| chr18 | 25556777 | 25556827 |
| chr18 | 40723476 | 40723526 |
| chr18 | 49340626 | 49340676 |
| chr18 | 49900860 | 49900910 |
| chr18 | 54469136 | 54469186 |
| chr18 | 54911314 | 54911364 |
| chr18 | 60314204 | 60314254 |
| chr18 | 62074819 | 62074869 |
| chr18 | 62682608 | 62682658 |
| chr18 | 67038604 | 67038654 |
| chr18 | 73832857 | 73832907 |
| chr18 | 74185717 | 74185767 |
| chr18 | 74185622 | 74185672 |
| chr19 | 1299145 | 1299195 |
| chr19 | 1646460 | 1646510 |
| chr19 | 2779151 | 2779201 |
| chr19 | 7427127 | 7427177 |
| chr19 | 8073567 | 8073617 |
| chr19 | 9135767 | 9135817 |
| chr19 | 16899134 | 16899184 |
| chr19 | 17475311 | 17475361 |
| chr19 | 36311614 | 36311664 |
| chr19 | 38505682 | 38505732 |
| chr10 | 104557250 | 104557300 |
| chr19 | 44231592 | 44231642 |
| chr10 | 108428542 | 108428592 |
| chr19 | 50804617 | 50804667 |
| chr19 | 51435136 | 51435186 |
| chr19 | 51518505 | 51518555 |
| chr19 | 51721882 | 51721932 |
| chr19 | 51734352 | 51734402 |
| chr19 | 51755106 | 51755156 |
| chr19 | 51839634 | 51839684 |
| chr19 | 53313249 | 53313299 |
| chr19 | 57583805 | 57583855 |
| chr10 | 113623780 | 113623830 |
| chr19 | 59231813 | 59231863 |
| chr19 | 59596809 | 59596859 |
| chr19 | 63435711 | 63435761 |
| chr1 | 3212413 | 3212463 |
| chr1 | 4195857 | 4195907 |
| chr1 | 4844173 | 4844223 |
| chr1 | 4981898 | 4981948 |
| chr1 | 5022830 | 5022880 |
| chr1 | 5588551 | 5588601 |
| chr1 | 7678680 | 7678730 |
| chr1 | 8106904 | 8106954 |
| chr1 | 8370136 | 8370186 |
| chr1 | 11177088 | 11177138 |
| chr1 | 17252240 | 17252290 |
| chr1 | 17907531 | 17907581 |
| chr1 | 19588040 | 19588090 |
| chr1 | 20414039 | 20414089 |
| chr1 | 28819162 | 28819212 |
| chr1 | 36402273 | 36402323 |
| chr1 | 38234531 | 38234581 |
| chr1 | 40385179 | 40385229 |
| chr1 | 42629117 | 42629167 |
| chr1 | 49103716 | 49103766 |
| chr1 | 49527992 | 49528042 |
| chr1 | 53182699 | 53182749 |
| chr1 | 53596568 | 53596618 |
| chr1 | 53840269 | 53840319 |
| chr1 | 56325651 | 56325701 |
| chr1 | 61496107 | 61496157 |
| chr1 | 68799890 | 68799940 |
| chr1 | 69175825 | 69175875 |
| chr1 | 71234370 | 71234420 |
| chr1 | 71585873 | 71585923 |
| chr1 | 74840872 | 74840922 |
| chr1 | 79814000 | 79814050 |
| chr1 | 85357258 | 85357308 |
| chr1 | 85357318 | 85357368 |
| chr1 | 89423581 | 89423631 |
| chr1 | 99533644 | 99533694 |
| chr1 | 109517637 | 109517687 |
| chr1 | 111516843 | 111516893 |
| chr1 | 155559572 | 155559622 |
| chr1 | 171969996 | 171970046 |
| chr10 | 7408920 | 7408970 |
| chr1 | 178238244 | 178238294 |
| chr1 | 179537310 | 179537360 |
| chr1 | 186732464 | 186732514 |
| chr1 | 195337604 | 195337654 |
| chr10 | 130344607 | 130344657 |
| chr1 | 208013082 | 208013132 |
| chr1 | 212821477 | 212821527 |
| chr1 | 213813940 | 213813990 |
| chr1 | 217588493 | 217588543 |
| chr1 | 220594274 | 220594324 |
| chr1 | 229472518 | 229472568 |
| chr1 | 229963425 | 229963475 |
| chr10 | 131398337 | 131398387 |
| chr1 | 241996481 | 241996531 |
| chr20 | 802400 | 802450 |
| chr20 | 3371328 | 3371378 |
| chr10 | 132740818 | 132740868 |
| chr20 | 6977179 | 6977229 |
| chr20 | 11978818 | 11978868 |
| chr20 | 12426160 | 12426210 |
| chr20 | 18719841 | 18719891 |
| chr20 | 19164953 | 19165003 |
| chr20 | 21719523 | 21719573 |
| chr20 | 30715808 | 30715858 |
| chr10 | 133573984 | 133574034 |
| chr20 | 37795091 | 37795141 |
| chr20 | 40705751 | 40705801 |
| chr20 | 41935256 | 41935306 |
| chr20 | 43356433 | 43356483 |
| chr10 | 133920653 | 133920703 |
| chr20 | 46033549 | 46033599 |
| chr20 | 46033343 | 46033393 |
| chr20 | 54455748 | 54455798 |
| chr20 | 60878152 | 60878202 |
| chr21 | 16377594 | 16377644 |
| chr21 | 21121219 | 21121269 |
| chr21 | 25684095 | 25684145 |
| chr21 | 26666236 | 26666286 |
| chr21 | 29574112 | 29574162 |
| chr21 | 30165196 | 30165246 |
| chr21 | 33290009 | 33290059 |
| chr21 | 39306627 | 39306677 |
| chr21 | 39710579 | 39710629 |
| chr21 | 40782219 | 40782269 |
| chr21 | 41353072 | 41353122 |
| chr21 | 42030147 | 42030197 |
| chr21 | 43841239 | 43841289 |
| chr21 | 43940646 | 43940696 |
| chr21 | 44854458 | 44854508 |
| chr21 | 46317397 | 46317447 |
| chr21 | 46432727 | 46432777 |
| chr21 | 46433136 | 46433186 |
| chr22 | 26992125 | 26992175 |
| chr22 | 31383600 | 31383650 |
| chr22 | 35585170 | 35585220 |
| chr22 | 41712815 | 41712865 |
| chr22 | 43171879 | 43171929 |
| chr22 | 45665754 | 45665804 |
| chr22 | 45708952 | 45709002 |
| chr22 | 45748610 | 45748660 |
| chr22 | 46101425 | 46101475 |
| chr22 | 46803563 | 46803613 |
| chr22 | 47803519 | 47803569 |
| chr22 | 48057909 | 48057959 |
| chrX | 17465100 | 17465150 |
| chrX | 20086619 | 20086669 |
| chrX | 22554826 | 22554876 |
| chrX | 22612177 | 22612227 |
| chrX | 23832987 | 23833037 |
| chr11 | 7062758 | 7062808 |
| chrX | 46301998 | 46302048 |
| chrX | 47554064 | 47554114 |
| chr11 | 8178181 | 8178231 |
| chrX | 71991359 | 71991409 |
| chrX | 91981633 | 91981683 |
| chrX | 112060891 | 112060941 |
| chrX | 122281490 | 122281540 |
| chrX | 130001775 | 130001825 |
| chrX | 130206232 | 130206282 |
| chrX | 143765113 | 143765163 |
| chrX | 150348778 | 150348828 |
| chrY | 6476963 | 6477013 |
| chr11 | 14279320 | 14279370 |
| chrY | 7125685 | 7125735 |
| chrY | 20330654 | 20330704 |
| chrY | 22684288 | 22684338 |
| chr2 | 306520 | 306570 |
| chr2 | 1885133 | 1885183 |
| chr2 | 1967072 | 1967122 |
| chr10 | 11438947 | 11438997 |
| chr2 | 3791064 | 3791114 |
| chr2 | 4728337 | 4728387 |
| chr2 | 7131952 | 7132002 |
| chr2 | 9359418 | 9359468 |
| chr2 | 9867616 | 9867666 |
| chr2 | 10139813 | 10139863 |
| chr2 | 16187978 | 16188028 |
| chr2 | 17990089 | 17990139 |
| chr2 | 21237468 | 21237518 |
| chr2 | 24745411 | 24745461 |
| chr2 | 38102124 | 38102174 |
| chr2 | 44367925 | 44367975 |
| chr2 | 47608025 | 47608075 |
| chr2 | 52650394 | 52650444 |
| chr2 | 61479885 | 61479935 |
| chr2 | 65520062 | 65520112 |
| chr2 | 68889480 | 68889530 |
| chr11 | 25615137 | 25615187 |
| chr2 | 71399646 | 71399696 |
| chr2 | 83562275 | 83562325 |
| chr2 | 87419465 | 87419515 |
| chr2 | 99541535 | 99541585 |
| chr2 | 99541479 | 99541529 |
| chr2 | 100220279 | 100220329 |
| chr2 | 102305463 | 102305513 |
| chr2 | 110650530 | 110650580 |
| chr11 | 32214292 | 32214342 |
| chr2 | 112600698 | 112600748 |
| chr2 | 113302667 | 113302717 |
| chr2 | 114666213 | 114666263 |
| chr2 | 117696100 | 117696150 |
| chr2 | 118876651 | 118876701 |
| chr2 | 119017236 | 119017286 |
| chr11 | 32593362 | 32593412 |
| chr2 | 128676950 | 128677000 |
| chr2 | 132034989 | 132035039 |
| chr2 | 132082474 | 132082524 |
| chr2 | 139205273 | 139205323 |
| chr2 | 140775550 | 140775600 |
| chr2 | 144889273 | 144889323 |
| chr2 | 153798978 | 153799028 |
| chr2 | 154886295 | 154886345 |
| chr11 | 34861570 | 34861620 |
| chr2 | 165651095 | 165651145 |
| chr2 | 167201731 | 167201781 |
| chr2 | 174487761 | 174487811 |
| chr11 | 37203237 | 37203287 |
| chr11 | 37943405 | 37943455 |
| chr2 | 185398017 | 185398067 |
| chr2 | 186474851 | 186474901 |
| chr2 | 186977083 | 186977133 |
| chr2 | 188363292 | 188363342 |
| chr2 | 191940414 | 191940464 |
| chr11 | 40004103 | 40004153 |
| chr2 | 209605296 | 209605346 |
| chr2 | 216896350 | 216896400 |
| chr2 | 216896575 | 216896625 |
| chr2 | 221513200 | 221513250 |
| chr2 | 222714529 | 222714579 |
| chr2 | 226604718 | 226604768 |
| chr2 | 233997498 | 233997548 |
| chr2 | 234758233 | 234758283 |
| chr11 | 45565486 | 45565536 |
| chr2 | 240618834 | 240618884 |
| chr2 | 241195575 | 241195625 |
| chr3 | 1655839 | 1655889 |
| chr3 | 3802307 | 3802357 |
| chr3 | 7585326 | 7585376 |
| chr3 | 11421035 | 11421085 |
| chr3 | 11822044 | 11822094 |
| chr3 | 17866246 | 17866296 |
| chr3 | 19120251 | 19120301 |
| chr3 | 21618881 | 21618931 |
| chr3 | 31525210 | 31525260 |
| chr3 | 36726994 | 36727044 |
| chr3 | 40869541 | 40869591 |
| chr3 | 44675959 | 44676009 |
| chr11 | 49732024 | 49732074 |
| chr3 | 47824561 | 47824611 |
| chr3 | 52723365 | 52723415 |
| chr3 | 58836981 | 58837031 |
| chr3 | 68631404 | 68631454 |
| chr3 | 70551990 | 70552040 |
| chr3 | 75436960 | 75437010 |
| chr3 | 75972487 | 75972537 |
| chr3 | 76025510 | 76025560 |
| chr3 | 77221874 | 77221924 |
| chr3 | 77981451 | 77981501 |
| chr11 | 58306356 | 58306406 |
| chr3 | 78730150 | 78730200 |
| chr3 | 81361775 | 81361825 |
| chr3 | 82650266 | 82650316 |
| chr3 | 104485115 | 104485165 |
| chr11 | 59459352 | 59459402 |
| chr3 | 106475678 | 106475728 |
| chr3 | 107860923 | 107860973 |
| chr3 | 112137678 | 112137728 |
| chr3 | 112136953 | 112137003 |
| chr3 | 120240851 | 120240901 |
| chr3 | 130424804 | 130424854 |
| chr3 | 137803978 | 137804028 |
| chr3 | 137911293 | 137911343 |
| chr10 | 12862314 | 12862364 |
| chr3 | 160793497 | 160793547 |
| chr3 | 177929670 | 177929720 |
| chr3 | 183334497 | 183334547 |
| chr10 | 12946369 | 12946419 |
| chr3 | 192463836 | 192463886 |
| chr3 | 193998891 | 193998941 |
| chr3 | 197833426 | 197833476 |
| chr4 | 4332138 | 4332188 |
| chr10 | 13294319 | 13294369 |
| chr4 | 22149967 | 22150017 |
| chr4 | 22750106 | 22750156 |
| chr4 | 26900261 | 26900311 |
| chr4 | 26924430 | 26924480 |
| chr4 | 28039035 | 28039085 |
| chr4 | 36905067 | 36905117 |
| chr4 | 39433527 | 39433577 |
| chr4 | 42305247 | 42305297 |
| chr4 | 60433133 | 60433183 |
| chr4 | 64554165 | 64554215 |
| chr4 | 68856811 | 68856861 |
| chr4 | 80217819 | 80217869 |
| chr4 | 83820490 | 83820540 |
| chr4 | 88444558 | 88444608 |
| chr4 | 89006727 | 89006777 |
| chr4 | 95719437 | 95719487 |
| chr4 | 103590803 | 103590853 |
| chr4 | 114151945 | 114151995 |
| chr4 | 114631876 | 114631926 |
| chr4 | 119755571 | 119755621 |
| chr4 | 120769156 | 120769206 |
| chr4 | 123335103 | 123335153 |
| chr4 | 124438221 | 124438271 |
| chr4 | 126552992 | 126553042 |
| chr4 | 131763135 | 131763185 |
| chr4 | 162765339 | 162765389 |
| chr4 | 179216596 | 179216646 |
| chr4 | 190753456 | 190753506 |
| chr5 | 1593694 | 1593744 |
| chr5 | 2072915 | 2072965 |
| chr5 | 2072789 | 2072839 |
| chr5 | 2557966 | 2558016 |
| chr5 | 3677977 | 3678027 |
| chr5 | 6932896 | 6932946 |
| chr11 | 69244130 | 69244180 |
| chr5 | 12701279 | 12701329 |
| chr5 | 13460833 | 13460883 |
| chr5 | 14586250 | 14586300 |
| chr5 | 16962395 | 16962445 |
| chr5 | 19187467 | 19187517 |
| chr5 | 19189055 | 19189105 |
| chr5 | 20662007 | 20662057 |
| chr5 | 23738276 | 23738326 |
| chr5 | 27892465 | 27892515 |
| chr5 | 29312782 | 29312832 |
| chr5 | 31594492 | 31594542 |
| chr5 | 34340955 | 34341005 |
| chr5 | 35676444 | 35676494 |
| chr5 | 39447101 | 39447151 |
| chr5 | 42705208 | 42705258 |
| chr5 | 50222077 | 50222127 |
| chr5 | 52725219 | 52725269 |
| chr5 | 54468271 | 54468321 |
| chr5 | 55325181 | 55325231 |
| chr5 | 55328011 | 55328061 |
| chr5 | 56611160 | 56611210 |
| chr5 | 56610980 | 56611030 |
| chr5 | 76141530 | 76141580 |
| chr5 | 77893969 | 77894019 |
| chr5 | 78246530 | 78246580 |
| chr5 | 82493950 | 82494000 |
| chr5 | 90726118 | 90726168 |
| chr5 | 94183670 | 94183720 |
| chr5 | 98002293 | 98002343 |
| chr5 | 100499751 | 100499801 |
| chr11 | 71689764 | 71689814 |
| chr11 | 71690117 | 71690167 |
| chr11 | 71690701 | 71690751 |
| chr5 | 116194075 | 116194125 |
| chr5 | 122159200 | 122159250 |
| chr5 | 127484253 | 127484303 |
| chr5 | 128279390 | 128279440 |
| chr5 | 130538202 | 130538252 |
| chr5 | 142618147 | 142618197 |
| chr5 | 142886871 | 142886921 |
| chr11 | 73894768 | 73894818 |
| chr5 | 150904173 | 150904223 |
| chr5 | 150977147 | 150977197 |
| chr5 | 158705564 | 158705614 |
| chr5 | 164509259 | 164509309 |
| chr5 | 164851956 | 164852006 |
| chr5 | 170497918 | 170497968 |
| chr5 | 172429660 | 172429710 |
| chr6 | 7894607 | 7894657 |
| chr6 | 11180622 | 11180672 |
| chr6 | 11750023 | 11750073 |
| chr6 | 18631144 | 18631194 |
| chr6 | 21278388 | 21278438 |
| chr6 | 23166551 | 23166601 |
| chr6 | 32733241 | 32733291 |
| chr6 | 33858828 | 33858878 |
| chr6 | 39158373 | 39158423 |
| chr6 | 47459498 | 47459548 |
| chr11 | 88304436 | 88304486 |
| chr6 | 69561804 | 69561854 |
| chr6 | 76241121 | 76241171 |
| chr6 | 79303459 | 79303509 |
| chr6 | 81764187 | 81764237 |
| chr6 | 81883614 | 81883664 |
| chr11 | 92396398 | 92396448 |
| chr6 | 97823329 | 97823379 |
| chr6 | 110586960 | 110587010 |
| chr6 | 113033877 | 113033927 |
| chr6 | 120363234 | 120363284 |
| chr6 | 128004232 | 128004282 |
| chr6 | 129277061 | 129277111 |
| chr6 | 131778119 | 131778169 |
| chr6 | 134437302 | 134437352 |
| chr6 | 136892550 | 136892600 |
| chr6 | 140204144 | 140204194 |
| chr6 | 140474705 | 140474755 |
| chr6 | 140822079 | 140822129 |
| chr6 | 146093820 | 146093870 |
| chr6 | 146092174 | 146092224 |
| chr6 | 149156857 | 149156907 |
| chr6 | 150771481 | 150771531 |
| chr6 | 152143796 | 152143846 |
| chr6 | 152366485 | 152366535 |
| chr6 | 152366196 | 152366246 |
| chr6 | 153274342 | 153274392 |
| chr6 | 156445010 | 156445060 |
| chr6 | 158188826 | 158188876 |
| chr6 | 162072018 | 162072068 |
| chr6 | 164598986 | 164599036 |
| chr11 | 103182580 | 103182630 |
| chr7 | 645167 | 645217 |
| chr10 | 16503100 | 16503150 |
| chr7 | 3058562 | 3058612 |
| chr7 | 4317485 | 4317535 |
| chr7 | 4325470 | 4325520 |
| chr11 | 109304111 | 109304161 |
| chr7 | 9078817 | 9078867 |
| chr7 | 13090869 | 13090919 |
| chr7 | 20780197 | 20780247 |
| chr7 | 28227376 | 28227426 |
| chr7 | 28957764 | 28957814 |
| chr7 | 36100764 | 36100814 |
| chr7 | 37249640 | 37249690 |
| chr10 | 16833375 | 16833425 |
| chr7 | 42499293 | 42499343 |
| chr7 | 47314335 | 47314385 |
| chr7 | 53277630 | 53277680 |
| chr7 | 56021668 | 56021718 |
| chr11 | 114893507 | 114893557 |
| chr7 | 70700973 | 70701023 |
| chr7 | 70832250 | 70832300 |
| chr7 | 70887096 | 70887146 |
| chr7 | 71549690 | 71549740 |
| chr7 | 72221502 | 72221552 |
| chr7 | 85261092 | 85261142 |
| chr7 | 92748719 | 92748769 |
| chr7 | 96773573 | 96773623 |
| chr7 | 97341975 | 97342025 |
| chr7 | 97930937 | 97930987 |
| chr7 | 98622121 | 98622171 |
| chr7 | 100720567 | 100720617 |
| chr7 | 100721170 | 100721220 |
| chr7 | 100841891 | 100841941 |
| chr7 | 108389817 | 108389867 |
| chr7 | 115782481 | 115782531 |
| chr7 | 116635908 | 116635958 |
| chr7 | 120878667 | 120878717 |
| chr7 | 121472273 | 121472323 |
| chr7 | 131845710 | 131845760 |
| chr11 | 118415010 | 118415060 |
| chr7 | 134203050 | 134203100 |
| chr7 | 135107266 | 135107316 |
| chr10 | 17516850 | 17516900 |
| chr7 | 137477211 | 137477261 |
| chr7 | 148435669 | 148435719 |
| chr7 | 153021703 | 153021753 |
| chr7 | 154451895 | 154451945 |
| chr7 | 156268675 | 156268725 |
| chr7 | 156268457 | 156268507 |
| chr7 | 158163649 | 158163699 |
| chr8 | 196756 | 196806 |
| chr11 | 120910436 | 120910486 |
| chr8 | 3497098 | 3497148 |
| chr8 | 10420439 | 10420489 |
| chr8 | 12658175 | 12658225 |
| chr8 | 14644106 | 14644156 |
| chr8 | 15394187 | 15394237 |
| chr8 | 16903837 | 16903887 |
| chr8 | 21224902 | 21224952 |
| chr11 | 122849124 | 122849174 |
| chr8 | 24115056 | 24115106 |
| chr11 | 123072192 | 123072242 |
| chr8 | 27809634 | 27809684 |
| chr8 | 30133875 | 30133925 |
| chr8 | 30174141 | 30174191 |
| chr8 | 30874375 | 30874425 |
| chr8 | 31166658 | 31166708 |
| chr8 | 33574521 | 33574571 |
| chr8 | 37495402 | 37495452 |
| chr8 | 56652156 | 56652206 |
| chr8 | 57531322 | 57531372 |
| chr8 | 58881323 | 58881373 |
| chr8 | 64498787 | 64498837 |
| chr8 | 85484953 | 85485003 |
| chr8 | 87539909 | 87539959 |
| chr11 | 126704776 | 126704826 |
| chr8 | 91586500 | 91586550 |
| chr8 | 111345140 | 111345190 |
| chr8 | 112393415 | 112393465 |
| chr8 | 116270696 | 116270746 |
| chr8 | 116838334 | 116838384 |
| chr8 | 122474439 | 122474489 |
| chr8 | 124680721 | 124680771 |
| chr8 | 127792715 | 127792765 |
| chr8 | 131439329 | 131439379 |
| chr8 | 132576368 | 132576418 |
| chr8 | 135686406 | 135686456 |
| chr8 | 137559036 | 137559086 |
| chr8 | 139797068 | 139797118 |
| chr8 | 139905464 | 139905514 |
| chr8 | 140835125 | 140835175 |
| chr11 | 130326971 | 130327021 |
| chr9 | 7351896 | 7351946 |
| chr11 | 132134187 | 132134237 |
| chr9 | 10161887 | 10161937 |
| chr9 | 16596905 | 16596955 |
| chr9 | 16907364 | 16907414 |
| chr9 | 17788675 | 17788725 |
| chr9 | 20086102 | 20086152 |
| chr9 | 21908394 | 21908444 |
| chr9 | 24714757 | 24714807 |
| chr9 | 26623809 | 26623859 |
| chr9 | 30022898 | 30022948 |
| chr9 | 31568607 | 31568657 |
| chr9 | 31714485 | 31714535 |
| chr9 | 38252030 | 38252080 |
| chr9 | 39237898 | 39237948 |
| chr9 | 41213594 | 41213644 |
| chr9 | 63665818 | 63665868 |
| chr9 | 80951538 | 80951588 |
| chr11 | 133851619 | 133851669 |
| chr9 | 87832548 | 87832598 |
| chr9 | 87911610 | 87911660 |
| chr9 | 91250072 | 91250122 |
| chr9 | 99921541 | 99921591 |
| chr9 | 106507549 | 106507599 |
| chr9 | 107736555 | 107736605 |
| chr9 | 113485125 | 113485175 |
| chr9 | 118660347 | 118660397 |
| chr9 | 122248827 | 122248877 |
| chr9 | 123920214 | 123920264 |
| chr9 | 128713706 | 128713756 |
| chr9 | 130240519 | 130240569 |
| chr9 | 133203778 | 133203828 |
| chr9 | 133763238 | 133763288 |
| chr9 | 134187415 | 134187465 |
| chr10 | 1066339 | 1066389 |
| chr12 | 9739054 | 9739104 |
| chr12 | 16683725 | 16683775 |
| chr12 | 17198287 | 17198337 |
| chr12 | 19022674 | 19022724 |
| chr12 | 25000045 | 25000095 |
| chr12 | 33662885 | 33662935 |
| chr12 | 33892301 | 33892351 |
| chr12 | 39767360 | 39767410 |
| chr12 | 41983462 | 41983512 |
| chr10 | 25343511 | 25343561 |
| chr12 | 53957670 | 53957720 |
| chr12 | 61191808 | 61191858 |
| chr12 | 62231614 | 62231664 |
| chr10 | 27987185 | 27987235 |
| chr12 | 62426679 | 62426729 |
| chr12 | 64700655 | 64700705 |
| chr12 | 68985178 | 68985228 |
| chr12 | 69544663 | 69544713 |
| chr10 | 28759765 | 28759815 |
| chr12 | 69738770 | 69738820 |
| chr12 | 75764879 | 75764929 |
| chr12 | 76763515 | 76763565 |
| chr12 | 81471420 | 81471470 |
| chr12 | 86677801 | 86677851 |
| chr10 | 29437955 | 29438005 |
| chr12 | 89632706 | 89632756 |
| chr12 | 95024436 | 95024486 |
| chr12 | 103250232 | 103250282 |
| chr12 | 104194657 | 104194707 |
| chr12 | 104649411 | 104649461 |
| chr10 | 30355749 | 30355799 |
| chr10 | 30571889 | 30571939 |
| chr12 | 113456838 | 113456888 |
| chr12 | 114795515 | 114795565 |
| chr12 | 116404850 | 116404900 |
| chr12 | 119185258 | 119185308 |
| chr12 | 123612332 | 123612382 |
| chr12 | 125406029 | 125406079 |
| chr12 | 125700319 | 125700369 |
| chr12 | 130579787 | 130579837 |
| chr13 | 24833257 | 24833307 |
| chr13 | 30255140 | 30255190 |
| chr13 | 39093542 | 39093592 |
| chr13 | 44799080 | 44799130 |
| chr13 | 51199299 | 51199349 |
| chr13 | 57208795 | 57208845 |
| chr13 | 57341338 | 57341388 |
| chr13 | 66480332 | 66480382 |
| chr13 | 71423621 | 71423671 |
| chr13 | 72445465 | 72445515 |
| chr13 | 72614089 | 72614139 |
| chr13 | 76297983 | 76298033 |
| chr13 | 83285692 | 83285742 |
| chr13 | 86870219 | 86870269 |
| chr13 | 90469480 | 90469530 |
| chr13 | 93624225 | 93624275 |
| chr13 | 96419543 | 96419593 |
| chr13 | 96479468 | 96479518 |
| chr13 | 97470666 | 97470716 |
| chr13 | 102174450 | 102174500 |
| chr13 | 103680574 | 103680624 |
| chr13 | 108611014 | 108611064 |
| chr13 | 112540959 | 112541009 |
| chr13 | 112663109 | 112663159 |
| chr14 | 25625906 | 25625956 |
| chr14 | 26998190 | 26998240 |
| chr14 | 27061331 | 27061381 |
| chr14 | 28819290 | 28819340 |
| chr14 | 31456669 | 31456719 |
| chr14 | 33186802 | 33186852 |
| chr14 | 33650029 | 33650079 |
| chr14 | 35663309 | 35663359 |
| chr14 | 50851666 | 50851716 |
| chr14 | 55861576 | 55861626 |
| chr14 | 62383989 | 62384039 |
| chr14 | 62992791 | 62992841 |
| chr14 | 62992590 | 62992640 |
| chr14 | 62992537 | 62992587 |
| chr14 | 65077682 | 65077732 |
| chr14 | 68287292 | 68287342 |
| chr14 | 68770504 | 68770554 |
| chr14 | 76813719 | 76813769 |
| chr14 | 87837040 | 87837090 |
| chr14 | 87836927 | 87836977 |
| chr14 | 88609259 | 88609309 |
| chr14 | 89882172 | 89882222 |
| chr14 | 91329544 | 91329594 |
| chr14 | 95725985 | 95726035 |
| chr10 | 56682870 | 56682920 |
| chr14 | 97751571 | 97751621 |
| chr14 | 99507267 | 99507317 |
| chr14 | 100565595 | 100565645 |
| chr14 | 103461211 | 103461261 |
| chr14 | 103742588 | 103742638 |
| chr14 | 105044041 | 105044091 |
| chr15 | 21626928 | 21626978 |
| chr15 | 33747774 | 33747824 |
| chr15 | 38023953 | 38024003 |
| chr15 | 38022802 | 38022852 |
| chr10 | 2470547 | 2470597 |
| chr15 | 46266739 | 46266789 |
| chr15 | 48903231 | 48903281 |
| chr15 | 54405149 | 54405199 |
| chr15 | 54404697 | 54404747 |
| chr15 | 55036113 | 55036163 |
| chr15 | 69202348 | 69202398 |
| chr15 | 70253685 | 70253735 |
| chr15 | 74973933 | 74973983 |
| chr15 | 79987446 | 79987496 |
| chr15 | 88229107 | 88229157 |
| chr15 | 90630924 | 90630974 |
| chr15 | 92440803 | 92440853 |
| chr15 | 93382980 | 93383030 |
| chr15 | 96711319 | 96711369 |
| chr15 | 98052588 | 98052638 |
| chr16 | 1786565 | 1786615 |
| chr10 | 3031800 | 3031850 |
| chr16 | 6234799 | 6234849 |
| chr16 | 8635088 | 8635138 |
| chr16 | 10607829 | 10607879 |
| chr16 | 12523437 | 12523487 |
| chr16 | 12879834 | 12879884 |
| chr16 | 13111964 | 13112014 |
| chr16 | 13549629 | 13549679 |
| chr16 | 16938176 | 16938226 |
| chr16 | 18273340 | 18273390 |
| chr16 | 18921356 | 18921406 |
| chr16 | 23682763 | 23682813 |
| chr16 | 25318826 | 25318876 |
| chr16 | 36142653 | 36142703 |
| chr16 | 47955794 | 47955844 |
| chr16 | 47955886 | 47955936 |
| chr16 | 50286274 | 50286324 |
| chr16 | 54070311 | 54070361 |
| chr16 | 59893668 | 59893718 |
| chr16 | 59895581 | 59895631 |
| chr16 | 64726423 | 64726473 |
| chr16 | 65514498 | 65514548 |
| chr16 | 65564315 | 65564365 |
| chr10 | 73524388 | 73524438 |
| chr10 | 73524915 | 73524965 |
| chr16 | 71040536 | 71040586 |
| chr16 | 77644386 | 77644436 |
| chr16 | 77756101 | 77756151 |
| chr16 | 83089844 | 83089894 |
| chr16 | 83828862 | 83828912 |
| chr16 | 83829759 | 83829809 |
| chr16 | 86525439 | 86525489 |
| chr16 | 88154523 | 88154573 |
| chr16 | 89471616 | 89471666 |
| chr17 | 166183 | 166233 |
| chr10 | 76934632 | 76934682 |
| chr17 | 10334296 | 10334346 |
| chr17 | 10395358 | 10395408 |
| chr17 | 11859589 | 11859639 |
| chr17 | 14134391 | 14134441 |

Table 15EF. Repeat Category THE1B-int

| | | |
|---|---|---|
| chr17 | 58985856 | 58985906 |
| chr17 | 68806705 | 68806755 |
| chr10 | 90222619 | 90222669 |
| chr18 | 25556777 | 25556827 |
| chr1 | 69175825 | 69175875 |
| chr1 | 89423581 | 89423631 |
| chr20 | 12426160 | 12426210 |
| chr21 | 43841239 | 43841289 |
| chrX | 91981633 | 91981683 |
| chrY | 20330654 | 20330704 |
| chrY | 22684288 | 22684338 |
| chr2 | 44367925 | 44367975 |
| chr2 | 47608025 | 47608075 |
| chr2 | 119017236 | 119017286 |
| chr2 | 132082474 | 132082524 |
| chr2 | 153798978 | 153799028 |
| chr11 | 37203237 | 37203287 |
| chr2 | 226604718 | 226604768 |
| chr11 | 49732024 | 49732074 |
| chr3 | 58836981 | 58837031 |
| chr11 | 58306356 | 58306406 |
| chr4 | 36905067 | 36905117 |
| chr4 | 162765339 | 162765389 |
| chr5 | 34340955 | 34341005 |
| chr5 | 35676444 | 35676494 |
| chr5 | 82493950 | 82494000 |
| chr5 | 130538202 | 130538252 |
| chr11 | 88304436 | 88304486 |
| chr6 | 69561804 | 69561854 |
| chr6 | 97823329 | 97823379 |
| chr8 | 16903837 | 16903887 |
| chr11 | 123072192 | 123072242 |
| chr8 | 85484953 | 85485003 |
| chr8 | 111345140 | 111345190 |
| chr8 | 112393415 | 112393465 |
| chr9 | 30022898 | 30022948 |
| chr12 | 33662885 | 33662935 |
| chr12 | 119185258 | 119185308 |
| chr13 | 76297983 | 76298033 |
| chr13 | 86870219 | 86870269 |
| chr14 | 105044041 | 105044091 |
| chr15 | 38022802 | 38022852 |
| chr15 | 46266739 | 46266789 |
| chr15 | 48903231 | 48903281 |
| chr15 | 54405149 | 54405199 |
| chr16 | 59895581 | 59895631 |

Table 15EG. Repeat Category THE1C

| | | |
|---|---|---|
| chr17 | 47636949 | 47636999 |
| chr17 | 59796751 | 59796801 |
| chr17 | 70937688 | 70937738 |
| chr17 | 71770126 | 71770176 |
| chr17 | 71769983 | 71770033 |
| chr17 | 75704434 | 75704484 |
| chr17 | 77242580 | 77242630 |
| chr17 | 80241373 | 80241423 |
| chr17 | 81671328 | 81671378 |
| chr18 | 3467435 | 3467485 |
| chr18 | 10880787 | 10880837 |
| chr18 | 10880553 | 10880603 |
| chr18 | 27651163 | 27651213 |
| chr18 | 34123316 | 34123366 |
| chr18 | 43966383 | 43966433 |
| chr18 | 46022379 | 46022429 |
| chr18 | 53362630 | 53362680 |
| chr18 | 59275841 | 59275891 |
| chr18 | 65006100 | 65006150 |
| chr18 | 69004879 | 69004929 |
| chr19 | 1498742 | 1498792 |
| chr19 | 5858131 | 5858181 |
| chr19 | 14674487 | 14674537 |
| chr19 | 15502696 | 15502746 |
| chr10 | 103154131 | 103154181 |
| chr19 | 16611216 | 16611266 |
| chr19 | 16739076 | 16739126 |
| chr19 | 19158816 | 19158866 |
| chr19 | 19158653 | 19158703 |
| chr19 | 38965314 | 38965364 |
| chr19 | 40384870 | 40384920 |
| chr10 | 105726634 | 105726684 |
| chr10 | 105726430 | 105726480 |
| chr19 | 49277897 | 49277947 |
| chr19 | 49277323 | 49277373 |
| chr19 | 49277174 | 49277224 |
| chr19 | 56968063 | 56968113 |
| chr10 | 113271742 | 113271792 |
| chr19 | 62513169 | 62513219 |
| chr1 | 1111178 | 1111228 |
| chr10 | 115253082 | 115253132 |
| chr1 | 4849482 | 4849532 |
| chr1 | 4884680 | 4884730 |
| chr1 | 8626060 | 8626110 |
| chr10 | 117868280 | 117868330 |
| chr1 | 11349731 | 11349781 |
| chr1 | 13370416 | 13370466 |
| chr1 | 14410031 | 14410081 |
| chr1 | 14925969 | 14926019 |
| chr1 | 17190858 | 17190908 |
| chr1 | 22161436 | 22161486 |
| chr1 | 22161959 | 22162009 |
| chr1 | 24139471 | 24139521 |
| chr1 | 26865623 | 26865673 |
| chr1 | 42883165 | 42883215 |
| chr1 | 51972234 | 51972284 |
| chr1 | 52401142 | 52401192 |
| chr1 | 68504022 | 68504072 |
| chr1 | 71111524 | 71111574 |
| chr1 | 82304581 | 82304631 |
| chr1 | 90539795 | 90539845 |
| chr1 | 90576122 | 90576172 |
| chr1 | 94974324 | 94974374 |
| chr1 | 99880378 | 99880428 |
| chr1 | 102879865 | 102879915 |
| chr1 | 103466771 | 103466821 |
| chr1 | 103466554 | 103466604 |
| chr1 | 105493440 | 105493490 |
| chr1 | 172743711 | 172743761 |
| chr10 | 7379746 | 7379796 |
| chr1 | 184823767 | 184823817 |
| chr1 | 189009555 | 189009605 |
| chr1 | 190218654 | 190218704 |
| chr10 | 129903512 | 129903562 |
| chr1 | 215737520 | 215737570 |
| chr1 | 221098520 | 221098570 |
| chr1 | 236612610 | 236612660 |
| chr1 | 237841373 | 237841423 |
| chr20 | 2604173 | 2604223 |
| chr20 | 6998114 | 6998164 |
| chr20 | 21044779 | 21044829 |
| chr20 | 36989239 | 36989289 |
| chr20 | 36989301 | 36989351 |
| chr20 | 45998497 | 45998547 |
| chr10 | 134321538 | 134321588 |
| chr20 | 62054459 | 62054509 |
| chr21 | 14578330 | 14578380 |
| chr21 | 18011777 | 18011827 |
| chr21 | 18440570 | 18440620 |
| chr21 | 19085399 | 19085449 |
| chr21 | 19936595 | 19936645 |
| chr11 | 328409 | 328459 |
| chr21 | 27277749 | 27277799 |
| chr21 | 33229797 | 33229847 |
| chr21 | 34161574 | 34161624 |
| chr21 | 36079690 | 36079740 |
| chr21 | 44015635 | 44015685 |
| chr22 | 26678574 | 26678624 |
| chr22 | 29507642 | 29507692 |
| chr22 | 30246874 | 30246924 |
| chr22 | 30937629 | 30937679 |
| chr22 | 32139915 | 32139965 |
| chr11 | 2314129 | 2314179 |
| chr22 | 39414817 | 39414867 |
| chr22 | 42886770 | 42886820 |
| chr22 | 47664811 | 47664861 |
| chrX | 18675482 | 18675532 |
| chrX | 23345481 | 23345531 |
| chrX | 34314194 | 34314244 |
| chrX | 69653256 | 69653306 |
| chrX | 94984346 | 94984396 |
| chrX | 103537075 | 103537125 |
| chrX | 117705092 | 117705142 |
| chrX | 123955784 | 123955834 |
| chrX | 126289963 | 126290013 |
| chrX | 147505293 | 147505343 |
| chr2 | 2377763 | 2377813 |
| chr2 | 9840685 | 9840735 |
| chr2 | 13234301 | 13234351 |
| chr2 | 15467661 | 15467711 |
| chr2 | 16957702 | 16957752 |
| chr2 | 16963579 | 16963629 |
| chr2 | 25084839 | 25084889 |
| chr2 | 30636170 | 30636220 |
| chr2 | 33143789 | 33143839 |
| chr2 | 43556443 | 43556493 |
| chr2 | 45792265 | 45792315 |
| chr2 | 48443015 | 48443065 |
| chr2 | 72100823 | 72100873 |
| chr2 | 75983835 | 75983885 |
| chr2 | 76517586 | 76517636 |
| chr2 | 76627852 | 76627902 |
| chr11 | 28931923 | 28931973 |
| chr2 | 98155727 | 98155777 |
| chr2 | 101450677 | 101450727 |
| chr2 | 120551745 | 120551795 |
| chr2 | 138298836 | 138298886 |
| chr2 | 138300527 | 138300577 |
| chr2 | 153447456 | 153447506 |
| chr2 | 157485698 | 157485748 |
| chr2 | 162189849 | 162189899 |
| chr2 | 166651450 | 166651500 |
| chr2 | 173446723 | 173446773 |
| chr2 | 185068466 | 185068516 |
| chr2 | 193555207 | 193555257 |
| chr11 | 39435511 | 39435561 |
| chr2 | 222866721 | 222866771 |
| chr2 | 228366029 | 228366079 |
| chr2 | 238274355 | 238274405 |
| chr2 | 242108191 | 242108241 |
| chr3 | 2121831 | 2121881 |
| chr3 | 3523836 | 3523886 |
| chr3 | 15963889 | 15963939 |
| chr3 | 25726329 | 25726379 |
| chr3 | 26559408 | 26559458 |
| chr3 | 34358519 | 34358569 |
| chr3 | 35394429 | 35394479 |
| chr3 | 35394180 | 35394230 |
| chr3 | 65494761 | 65494811 |
| chr3 | 78636160 | 78636210 |
| chr3 | 80504015 | 80504065 |
| chr3 | 80540590 | 80540640 |
| chr3 | 89938698 | 89938748 |
| chr3 | 96470305 | 96470355 |
| chr3 | 151746498 | 151746548 |
| chr3 | 169182658 | 169182708 |
| chr3 | 189820271 | 189820321 |
| chr3 | 190419862 | 190419912 |
| chr3 | 192017425 | 192017475 |
| chr3 | 197913520 | 197913570 |
| chr4 | 6244287 | 6244337 |
| chr4 | 18483164 | 18483214 |
| chr4 | 21801802 | 21801852 |
| chr4 | 22540705 | 22540755 |
| chr4 | 26430069 | 26430119 |
| chr4 | 31779743 | 31779793 |
| chr4 | 36356613 | 36356663 |
| chr4 | 39708048 | 39708098 |
| chr4 | 65323790 | 65323840 |
| chr4 | 65523043 | 65523093 |
| chr4 | 79525456 | 79525506 |
| chr4 | 88818390 | 88818440 |
| chr4 | 89543962 | 89544012 |
| chr4 | 105268225 | 105268275 |
| chr4 | 106744571 | 106744621 |
| chr4 | 120030051 | 120030101 |
| chr4 | 124765024 | 124765074 |
| chr4 | 134017331 | 134017381 |
| chr4 | 148857810 | 148857860 |
| chr4 | 151683961 | 151684011 |
| chr4 | 151838704 | 151838754 |
| chr4 | 153965943 | 153965993 |
| chr4 | 157109616 | 157109666 |
| chr4 | 164763312 | 164763362 |
| chr4 | 166272578 | 166272628 |
| chr4 | 171788790 | 171788840 |
| chr4 | 181350801 | 181350851 |
| chr4 | 182747168 | 182747218 |
| chr4 | 184430171 | 184430221 |
| chr5 | 5135821 | 5135871 |
| chr5 | 10114896 | 10114946 |
| chr5 | 21188822 | 21188872 |
| chr5 | 21762636 | 21762686 |
| chr5 | 29219326 | 29219376 |
| chr5 | 41326950 | 41327000 |
| chr5 | 59968543 | 59968593 |
| chr5 | 63749951 | 63750001 |
| chr5 | 71230955 | 71231005 |
| chr5 | 80385193 | 80385243 |
| chr5 | 85984483 | 85984533 |
| chr11 | 71886662 | 71886712 |
| chr5 | 119102824 | 119102874 |
| chr5 | 119822132 | 119822182 |
| chr5 | 131102301 | 131102351 |
| chr5 | 154221110 | 154221160 |
| chr5 | 171682541 | 171682591 |
| chr5 | 175033526 | 175033576 |
| chr11 | 79277791 | 79277841 |
| chr6 | 19396981 | 19397031 |
| chr6 | 22335432 | 22335482 |
| chr11 | 82095823 | 82095873 |
| chr6 | 38350720 | 38350770 |
| chr6 | 78914374 | 78914424 |
| chr6 | 82865864 | 82865914 |
| chr11 | 92234017 | 92234067 |
| chr6 | 88276143 | 88276193 |
| chr6 | 124062745 | 124062795 |
| chr6 | 131069295 | 131069345 |
| chr6 | 135018284 | 135018334 |
| chr6 | 147372997 | 147373047 |
| chr6 | 162514826 | 162514876 |
| chr6 | 164857617 | 164857667 |
| chr6 | 166580567 | 166580617 |
| chr6 | 168234169 | 168234219 |
| chr6 | 169002116 | 169002166 |
| chr7 | 1382434 | 1382484 |
| chr7 | 6418626 | 6418676 |
| chr7 | 12577998 | 12578048 |
| chr7 | 26420224 | 26420274 |
| chr7 | 26420115 | 26420165 |
| chr7 | 34295115 | 34295165 |
| chr10 | 16844169 | 16844219 |
| chr7 | 70156268 | 70156318 |
| chr7 | 70411350 | 70411400 |
| chr7 | 70458206 | 70458256 |
| chr7 | 77059161 | 77059211 |
| chr7 | 80229375 | 80229425 |
| chr7 | 81250549 | 81250599 |
| chr7 | 81250418 | 81250468 |
| chr7 | 103231387 | 103231437 |
| chr7 | 109874535 | 109874585 |
| chr7 | 109873879 | 109873929 |
| chr7 | 114335151 | 114335201 |
| chr7 | 152865004 | 152865054 |
| chr7 | 153186761 | 153186811 |
| chr7 | 156992896 | 156992946 |
| chr8 | 20892229 | 20892279 |
| chr8 | 23390529 | 23390579 |
| chr8 | 32692131 | 32692181 |
| chr8 | 36060383 | 36060433 |
| chr8 | 37969979 | 37970029 |
| chr8 | 90454413 | 90454463 |
| chr8 | 92391559 | 92391609 |
| chr8 | 93846504 | 93846554 |
| chr8 | 99641042 | 99641092 |
| chr8 | 99640925 | 99640975 |
| chr8 | 102319454 | 102319504 |
| chr8 | 113724457 | 113724507 |
| chr10 | 19046603 | 19046653 |
| chr8 | 120260590 | 120260640 |
| chr8 | 120511484 | 120511534 |
| chr8 | 121028932 | 121028982 |
| chr8 | 122755841 | 122755891 |
| chr8 | 137041338 | 137041388 |
| chr8 | 138102901 | 138102951 |
| chr9 | 17112347 | 17112397 |
| chr9 | 23250170 | 23250220 |
| chr9 | 30232420 | 30232470 |
| chr9 | 32372950 | 32373000 |
| chr9 | 80494479 | 80494529 |
| chr9 | 82177141 | 82177191 |
| chr9 | 134021015 | 134021065 |
| chr12 | 22312918 | 22312968 |
| chr12 | 44691388 | 44691438 |
| chr12 | 50562628 | 50562678 |
| chr12 | 54329295 | 54329345 |
| chr12 | 55464978 | 55465028 |
| chr12 | 56891407 | 56891457 |
| chr12 | 58293089 | 58293139 |
| chr12 | 58292495 | 58292545 |
| chr12 | 68651706 | 68651756 |
| chr12 | 101347960 | 101348010 |
| chr12 | 103309216 | 103309266 |
| chr12 | 103309123 | 103309173 |
| chr12 | 111867216 | 111867266 |
| chr12 | 121083009 | 121083059 |
| chr12 | 126398245 | 126398295 |
| chr13 | 28786641 | 28786691 |
| chr13 | 51556435 | 51556485 |
| chr13 | 54879434 | 54879484 |
| chr13 | 85212997 | 85213047 |
| chr13 | 86267888 | 86267938 |
| chr13 | 97545414 | 97545464 |
| chr13 | 99421029 | 99421079 |
| chr13 | 109534311 | 109534361 |
| chr14 | 43660329 | 43660379 |
| chr14 | 45303870 | 45303920 |
| chr14 | 45673413 | 45673463 |
| chr14 | 54971915 | 54971965 |
| chr14 | 63764871 | 63764921 |
| chr14 | 65189705 | 65189755 |
| chr14 | 67742249 | 67742299 |
| chr14 | 69204548 | 69204598 |
| chr14 | 71843915 | 71843965 |
| chr14 | 75633597 | 75633647 |
| chr14 | 79401383 | 79401433 |
| chr14 | 87921362 | 87921412 |
| chr10 | 55084094 | 55084144 |
| chr14 | 90619951 | 90620001 |
| chr10 | 56467985 | 56468035 |
| chr14 | 97892506 | 97892556 |
| chr15 | 31295823 | 31295873 |
| chr15 | 34260484 | 34260534 |
| chr10 | 63406105 | 63406155 |
| chr15 | 51149756 | 51149806 |
| chr15 | 53516874 | 53516924 |
| chr15 | 64580015 | 64580065 |
| chr15 | 65993830 | 65993880 |
| chr15 | 75955973 | 75956023 |
| chr15 | 95406942 | 95406992 |
| chr16 | 40336 | 40386 |
| chr16 | 2875784 | 2875834 |
| chr16 | 9541076 | 9541126 |
| chr16 | 21094347 | 21094397 |
| chr16 | 46350455 | 46350505 |
| chr16 | 59018412 | 59018462 |
| chr16 | 63867309 | 63867359 |
| chr16 | 80483988 | 80484038 |
| chr16 | 83331241 | 83331291 |
| chr16 | 86686199 | 86686249 |
| chr16 | 89513481 | 89513531 |
| chr17 | 1401598 | 1401648 |
| chr17 | 1934855 | 1934905 |
| chr17 | 2757948 | 2757998 |
| chr10 | 76474112 | 76474162 |
| chr17 | 5723278 | 5723328 |
| chr17 | 6501641 | 6501691 |
| chr10 | 78796541 | 78796591 |
| chr10 | 79207401 | 79207451 |

Table 15EH. Repeat Category THE1D

| | | |
|---|---|---|
| chr17 | 48069632 | 48069682 |
| chr17 | 55703817 | 55703867 |
| chr17 | 56679818 | 56679868 |
| chr17 | 62017864 | 62017914 |
| chr17 | 62985967 | 62986017 |
| chr17 | 63229393 | 63229443 |
| chr17 | 65546067 | 65546117 |
| chr17 | 67146227 | 67146277 |
| chr17 | 67161361 | 67161411 |
| chr17 | 70550434 | 70550484 |
| chr17 | 77903258 | 77903308 |
| chr17 | 78758576 | 78758626 |
| chr10 | 91955066 | 91955116 |
| chr18 | 11359973 | 11360023 |
| chr10 | 94729948 | 94729998 |
| chr10 | 95007433 | 95007483 |
| chr18 | 23575893 | 23575943 |
| chr18 | 28066112 | 28066162 |
| chr18 | 32073297 | 32073347 |
| chr18 | 41444603 | 41444653 |
| chr18 | 41447177 | 41447227 |
| chr18 | 41941155 | 41941205 |
| chr18 | 50784563 | 50784613 |
| chr18 | 51961457 | 51961507 |
| chr10 | 97928901 | 97928951 |
| chr18 | 59429155 | 59429205 |
| chr18 | 65651125 | 65651175 |
| chr18 | 66268772 | 66268822 |
| chr18 | 74816367 | 74816417 |
| chr19 | 2779151 | 2779201 |
| chr19 | 11292980 | 11293030 |
| chr19 | 12929849 | 12929899 |
| chr19 | 41609387 | 41609437 |
| chr19 | 44106665 | 44106715 |
| chr19 | 45145464 | 45145514 |
| chr19 | 47239676 | 47239726 |
| chr19 | 47239756 | 47239806 |
| chr19 | 48646900 | 48646950 |
| chr19 | 51377888 | 51377938 |
| chr19 | 51772260 | 51772310 |
| chr19 | 54168230 | 54168280 |
| chr19 | 54921140 | 54921190 |
| chr19 | 59224969 | 59225019 |
| chr19 | 60967927 | 60967977 |
| chr1 | 7645020 | 7645070 |
| chr1 | 11432434 | 11432484 |
| chr1 | 13350247 | 13350297 |
| chr1 | 17412328 | 17412378 |
| chr1 | 21188955 | 21189005 |
| chr1 | 26865623 | 26865673 |
| chr1 | 32549707 | 32549757 |
| chr10 | 121037911 | 121037961 |
| chr1 | 37339156 | 37339206 |
| chr10 | 121367901 | 121367951 |
| chr1 | 53041408 | 53041458 |
| chr1 | 62183527 | 62183577 |
| chr1 | 70221167 | 70221217 |
| chr1 | 70297151 | 70297201 |
| chr1 | 73442809 | 73442859 |
| chr1 | 86871280 | 86871330 |
| chr1 | 86871958 | 86872008 |
| chr1 | 87460667 | 87460717 |
| chr1 | 91046540 | 91046590 |
| chr1 | 93345442 | 93345492 |
| chr1 | 93889011 | 93889061 |
| chr1 | 100918633 | 100918683 |
| chr1 | 101756050 | 101756100 |
| chr1 | 102510060 | 102510110 |
| chr1 | 150070877 | 150070927 |
| chr10 | 128348785 | 128348835 |
| chr1 | 159100466 | 159100516 |
| chr1 | 160467872 | 160467922 |
| chr1 | 191144825 | 191144875 |
| chr1 | 192320899 | 192320949 |
| chr1 | 200968211 | 200968261 |
| chr1 | 215143042 | 215143092 |
| chr1 | 228601973 | 228602023 |
| chr1 | 228972793 | 228972843 |
| chr1 | 231297636 | 231297686 |
| chr1 | 231915888 | 231915938 |
| chr10 | 7665614 | 7665664 |
| chr10 | 132725774 | 132725824 |
| chr20 | 5648083 | 5648133 |
| chr20 | 33850794 | 33850844 |
| chr20 | 33850627 | 33850677 |
| chr20 | 36429491 | 36429541 |
| chr20 | 38392612 | 38392662 |
| chr20 | 41089142 | 41089192 |
| chr20 | 46246626 | 46246676 |
| chr20 | 53360942 | 53360992 |
| chr20 | 59694976 | 59695026 |
| chr21 | 14723378 | 14723428 |
| chr21 | 30930399 | 30930449 |
| chr21 | 38165799 | 38165849 |
| chr21 | 40998192 | 40998242 |
| chr21 | 43456370 | 43456420 |
| chr21 | 46284419 | 46284469 |
| chr22 | 16954326 | 16954376 |
| chr11 | 1326497 | 1326547 |
| chr22 | 27884289 | 27884339 |
| chr11 | 2416073 | 2416123 |
| chr22 | 41829262 | 41829312 |
| chr22 | 46431520 | 46431570 |
| chrX | 2978061 | 2978111 |
| chrX | 4684361 | 4684411 |
| chrX | 8471941 | 8471991 |
| chr11 | 6538175 | 6538225 |
| chrX | 54134301 | 54134351 |
| chr10 | 11071454 | 11071504 |
| chrX | 70298556 | 70298606 |
| chrX | 74927522 | 74927572 |
| chrX | 77724057 | 77724107 |
| chr11 | 9094903 | 9094953 |
| chrX | 84113053 | 84113103 |
| chrX | 86117641 | 86117691 |
| chrX | 88572783 | 88572833 |
| chrX | 94492126 | 94492176 |
| chrX | 95760737 | 95760787 |
| chrX | 100223831 | 100223881 |
| chrX | 110650889 | 110650939 |
| chrX | 117586594 | 117586644 |
| chrX | 119765572 | 119765622 |
| chr11 | 10816251 | 10816301 |
| chr11 | 12007232 | 12007282 |
| chrX | 136405028 | 136405078 |
| chrX | 142191397 | 142191447 |
| chrX | 145517100 | 145517150 |
| chr11 | 12975399 | 12975449 |
| chr11 | 12975344 | 12975394 |
| chrX | 152357970 | 152358020 |
| chrY | 2721519 | 2721569 |
| chrY | 3524266 | 3524316 |
| chrY | 3694734 | 3694784 |
| chr11 | 14842298 | 14842348 |
| chr2 | 5588728 | 5588778 |
| chr2 | 11112249 | 11112299 |
| chr2 | 20047873 | 20047923 |
| chr2 | 20047781 | 20047831 |
| chr2 | 25972860 | 25972910 |
| chr2 | 35832226 | 35832276 |
| chr2 | 44349441 | 44349491 |
| chr2 | 45311857 | 45311907 |
| chr2 | 46408570 | 46408620 |
| chr2 | 50034204 | 50034254 |
| chr2 | 53710875 | 53710925 |
| chr2 | 54595758 | 54595808 |
| chr2 | 56880138 | 56880188 |
| chr11 | 24400944 | 24400994 |
| chr2 | 76142161 | 76142211 |
| chr2 | 86468772 | 86468822 |
| chr11 | 29710195 | 29710245 |
| chr2 | 102391866 | 102391916 |
| chr2 | 102391749 | 102391799 |
| chr2 | 120789006 | 120789056 |
| chr2 | 122178407 | 122178457 |
| chr2 | 131749860 | 131749910 |
| chr2 | 136371215 | 136371265 |
| chr2 | 142792967 | 142793017 |
| chr2 | 144000301 | 144000351 |
| chr2 | 167367496 | 167367546 |
| chr2 | 186190981 | 186191031 |
| chr2 | 196446537 | 196446587 |
| chr2 | 198423202 | 198423252 |
| chr11 | 41461409 | 41461459 |
| chr2 | 211339566 | 211339616 |
| chr2 | 216530365 | 216530415 |
| chr2 | 220755499 | 220755549 |
| chr2 | 221941094 | 221941144 |
| chr2 | 222196578 | 222196628 |
| chr2 | 224563640 | 224563690 |
| chr2 | 227697537 | 227697587 |
| chr2 | 233036508 | 233036558 |
| chr2 | 236074484 | 236074534 |
| chr2 | 238276865 | 238276915 |
| chr2 | 238943954 | 238944004 |
| chr2 | 238943884 | 238943934 |
| chr3 | 5348035 | 5348085 |
| chr3 | 11434869 | 11434919 |
| chr3 | 31105738 | 31105788 |
| chr3 | 41756502 | 41756552 |
| chr3 | 41756351 | 41756401 |
| chr3 | 51861954 | 51862004 |
| chr3 | 55753694 | 55753744 |
| chr3 | 61655195 | 61655245 |
| chr3 | 63207815 | 63207865 |
| chr3 | 65045379 | 65045429 |
| chr11 | 58173467 | 58173517 |
| chr3 | 85500278 | 85500328 |
| chr3 | 101289390 | 101289440 |
| chr3 | 104787959 | 104788009 |
| chr3 | 118806087 | 118806137 |
| chr3 | 141194320 | 141194370 |
| chr3 | 145608199 | 145608249 |
| chr3 | 165218910 | 165218960 |
| chr3 | 169357566 | 169357616 |
| chr3 | 171846734 | 171846784 |
| chr3 | 188030708 | 188030758 |
| chr11 | 63139942 | 63139992 |
| chr3 | 194195897 | 194195947 |
| chr3 | 194797247 | 194797297 |
| chr4 | 3466926 | 3466976 |
| chr4 | 3467668 | 3467718 |
| chr4 | 4968519 | 4968569 |
| chr4 | 7670849 | 7670899 |
| chr4 | 14377301 | 14377351 |
| chr4 | 22863337 | 22863387 |
| chr4 | 34130421 | 34130471 |
| chr4 | 34680755 | 34680805 |
| chr4 | 35233686 | 35233736 |
| chr4 | 38119008 | 38119058 |
| chr4 | 41284999 | 41285049 |
| chr4 | 43335826 | 43335876 |
| chr4 | 52879614 | 52879664 |
| chr4 | 52879456 | 52879506 |
| chr10 | 13372080 | 13372130 |
| chr4 | 68572670 | 68572720 |
| chr4 | 72376738 | 72376788 |
| chr4 | 73345511 | 73345561 |
| chr4 | 82747687 | 82747737 |
| chr4 | 85506141 | 85506191 |
| chr4 | 89405601 | 89405651 |
| chr4 | 89405262 | 89405312 |
| chr4 | 94906032 | 94906082 |
| chr4 | 113182652 | 113182702 |
| chr4 | 121162309 | 121162359 |
| chr4 | 121753255 | 121753305 |
| chr4 | 131969982 | 131970032 |
| chr4 | 134324249 | 134324299 |
| chr4 | 164865357 | 164865407 |
| chr4 | 164987585 | 164987635 |
| chr4 | 167497710 | 167497760 |
| chr4 | 170153632 | 170153682 |
| chr4 | 191613838 | 191613888 |
| chr5 | 2906653 | 2906703 |
| chr5 | 3590738 | 3590788 |
| chr5 | 20828932 | 20828982 |
| chr5 | 21993397 | 21993447 |
| chr5 | 23252956 | 23253006 |
| chr5 | 23489899 | 23489949 |
| chr5 | 23927213 | 23927263 |
| chr5 | 29758633 | 29758683 |
| chr11 | 69763113 | 69763163 |
| chr5 | 32508931 | 32508981 |
| chr11 | 69887353 | 69887403 |
| chr5 | 35560324 | 35560374 |
| chr5 | 39031819 | 39031869 |
| chr5 | 40491778 | 40491828 |
| chr5 | 44884357 | 44884407 |
| chr5 | 45862330 | 45862380 |
| chr5 | 56981829 | 56981879 |
| chr5 | 56981397 | 56981447 |
| chr5 | 56995986 | 56996036 |
| chr5 | 62320051 | 62320101 |
| chr5 | 68557784 | 68557834 |
| chr5 | 68591236 | 68591286 |
| chr11 | 70471508 | 70471558 |
| chr11 | 70556198 | 70556248 |
| chr5 | 75094719 | 75094769 |
| chr5 | 75094792 | 75094842 |
| chr5 | 80537325 | 80537375 |
| chr5 | 84783628 | 84783678 |
| chr5 | 86560281 | 86560331 |
| chr5 | 90593429 | 90593479 |
| chr5 | 100568126 | 100568176 |
| chr5 | 106422888 | 106422938 |
| chr5 | 117619260 | 117619310 |
| chr5 | 117736291 | 117736341 |
| chr5 | 141480312 | 141480362 |
| chr5 | 155716639 | 155716689 |
| chr5 | 156934807 | 156934857 |
| chr11 | 74384051 | 74384101 |
| chr5 | 157443683 | 157443733 |
| chr5 | 157445895 | 157445945 |
| chr5 | 174988631 | 174988681 |
| chr11 | 76352926 | 76352976 |
| chr11 | 76481368 | 76481418 |
| chr5 | 179802331 | 179802381 |
| chr6 | 3730305 | 3730355 |
| chr6 | 7328002 | 7328052 |
| chr6 | 9080274 | 9080324 |
| chr6 | 15595691 | 15595741 |
| chr6 | 15595571 | 15595621 |
| chr6 | 21233228 | 21233278 |
| chr6 | 24329230 | 24329280 |
| chr11 | 81265431 | 81265481 |
| chr6 | 27073075 | 27073125 |
| chr6 | 34403893 | 34403943 |
| chr6 | 35774953 | 35775003 |
| chr6 | 38350720 | 38350770 |
| chr6 | 46641817 | 46641867 |
| chr6 | 52797671 | 52797721 |
| chr6 | 57681279 | 57681329 |
| chr6 | 66632728 | 66632778 |
| chr6 | 69530768 | 69530818 |
| chr6 | 71333243 | 71333293 |
| chr6 | 75504907 | 75504957 |
| chr6 | 81903529 | 81903579 |
| chr6 | 83001016 | 83001066 |
| chr6 | 97198957 | 97199007 |
| chr6 | 120307337 | 120307387 |
| chr6 | 123209767 | 123209817 |
| chr6 | 126606182 | 126606232 |
| chr6 | 137805431 | 137805481 |
| chr6 | 139003253 | 139003303 |
| chr6 | 139149367 | 139149417 |
| chr6 | 139793550 | 139793600 |
| chr10 | 15608417 | 15608467 |
| chr10 | 15608232 | 15608282 |
| chr6 | 151317472 | 151317522 |
| chr11 | 100196111 | 100196161 |
| chr6 | 153188489 | 153188539 |
| chr6 | 153804560 | 153804610 |
| chr6 | 160878887 | 160878937 |
| chr6 | 161091126 | 161091176 |
| chr6 | 164269279 | 164269329 |
| chr6 | 164269165 | 164269215 |
| chr6 | 165526450 | 165526500 |
| chr11 | 103007823 | 103007873 |
| chr6 | 166207679 | 166207729 |
| chr11 | 103245047 | 103245097 |
| chr7 | 2653220 | 2653270 |
| chr7 | 3753284 | 3753334 |
| chr7 | 4401802 | 4401852 |
| chr7 | 4859467 | 4859517 |
| chr7 | 5597813 | 5597863 |
| chr7 | 12107515 | 12107565 |
| chr7 | 18635316 | 18635366 |
| chr7 | 22322101 | 22322151 |
| chr7 | 32470528 | 32470578 |
| chr7 | 32580145 | 32580195 |
| chr7 | 37443951 | 37444001 |
| chr7 | 39622435 | 39622485 |
| chr7 | 44973021 | 44973071 |
| chr7 | 56978683 | 56978733 |
| chr7 | 57287551 | 57287601 |
| chr7 | 63120931 | 63120981 |
| chr7 | 64214264 | 64214314 |
| chr11 | 115060752 | 115060802 |
| chr7 | 71087236 | 71087286 |
| chr7 | 71282581 | 71282631 |
| chr7 | 83084432 | 83084482 |
| chr7 | 83563346 | 83563396 |
| chr7 | 99800870 | 99800920 |
| chr7 | 99800634 | 99800684 |
| chr7 | 101285405 | 101285455 |
| chr7 | 104801841 | 104801891 |
| chr7 | 123724604 | 123724654 |
| chr7 | 130423235 | 130423285 |
| chr7 | 132682841 | 132682891 |
| chr7 | 143748226 | 143748276 |
| chr7 | 143962506 | 143962556 |
| chr7 | 146503874 | 146503924 |
| chr7 | 147450479 | 147450529 |
| chr7 | 152272636 | 152272686 |
| chr8 | 1113338 | 1113388 |
| chr8 | 1738797 | 1738847 |
| chr8 | 2041578 | 2041628 |
| chr8 | 3125027 | 3125077 |
| chr8 | 5815959 | 5816009 |
| chr8 | 11118303 | 11118353 |
| chr8 | 11874918 | 11874968 |
| chr8 | 14540955 | 14541005 |
| chr8 | 14718930 | 14718980 |
| chr8 | 17298645 | 17298695 |
| chr8 | 17921747 | 17921797 |
| chr8 | 19676904 | 19676954 |
| chr8 | 20676624 | 20676674 |
| chr8 | 23924581 | 23924631 |
| chr8 | 29920230 | 29920280 |
| chr8 | 31122643 | 31122693 |
| chr8 | 41300480 | 41300530 |
| chr8 | 47980051 | 47980101 |
| chr8 | 70844439 | 70844489 |
| chr8 | 76491538 | 76491588 |
| chr8 | 76626325 | 76626375 |
| chr8 | 76628152 | 76628202 |
| chr8 | 77300318 | 77300368 |
| chr8 | 87031226 | 87031276 |
| chr8 | 88780488 | 88780538 |
| chr8 | 111346851 | 111346901 |
| chr8 | 115680711 | 115680761 |
| chr8 | 115844280 | 115844330 |
| chr8 | 125140793 | 125140843 |
| chr8 | 135633941 | 135633991 |
| chr8 | 138029501 | 138029551 |
| chr8 | 141044675 | 141044725 |
| chr8 | 141044439 | 141044489 |
| chr9 | 4534565 | 4534615 |
| chr9 | 16923052 | 16923102 |
| chr9 | 16973407 | 16973457 |
| chr9 | 17359602 | 17359652 |
| chr9 | 30606051 | 30606101 |
| chr9 | 40667587 | 40667637 |
| chr9 | 74527285 | 74527335 |
| chr10 | 21072345 | 21072395 |
| chr9 | 94162449 | 94162499 |
| chr9 | 101625822 | 101625872 |
| chr9 | 110675809 | 110675859 |
| chr9 | 115060157 | 115060207 |
| chr9 | 115059518 | 115059568 |
| chr9 | 119205835 | 119205885 |
| chr9 | 119401902 | 119401952 |
| chr9 | 120243950 | 120244000 |
| chr9 | 124399947 | 124399997 |
| chr10 | 21874435 | 21874485 |
| chr12 | 8010771 | 8010821 |
| chr12 | 26129464 | 26129514 |
| chr12 | 38883757 | 38883807 |
| chr12 | 41355906 | 41355956 |
| chr12 | 43745316 | 43745366 |
| chr12 | 43745509 | 43745559 |
| chr12 | 46553856 | 46553906 |
| chr12 | 47863804 | 47863854 |
| chr12 | 56050168 | 56050218 |
| chr12 | 58647722 | 58647772 |
| chr12 | 59136119 | 59136169 |
| chr12 | 67828091 | 67828141 |
| chr12 | 68104726 | 68104776 |
| chr12 | 72705579 | 72705629 |
| chr12 | 73238994 | 73239044 |
| chr12 | 78898913 | 78898963 |
| chr12 | 81461078 | 81461128 |
| chr12 | 83261370 | 83261420 |
| chr12 | 89608008 | 89608058 |
| chr12 | 89632706 | 89632756 |
| chr12 | 92519973 | 92520023 |
| chr12 | 92528285 | 92528335 |
| chr12 | 108540193 | 108540243 |
| chr12 | 114891204 | 114891254 |
| chr12 | 122681491 | 122681541 |
| chr12 | 123507139 | 123507189 |
| chr10 | 33773888 | 33773938 |
| chr12 | 124447138 | 124447188 |
| chr12 | 124532238 | 124532288 |
| chr12 | 128660138 | 128660188 |
| chr13 | 26562555 | 26562605 |
| chr13 | 32034086 | 32034136 |
| chr13 | 34821242 | 34821292 |
| chr13 | 39482195 | 39482245 |
| chr13 | 39768990 | 39769040 |
| chr10 | 42235300 | 42235350 |
| chr13 | 57984823 | 57984873 |
| chr13 | 60820375 | 60820425 |
| chr13 | 64604546 | 64604596 |
| chr13 | 68227740 | 68227790 |
| chr13 | 70526253 | 70526303 |
| chr13 | 78795969 | 78796019 |
| chr13 | 79518935 | 79518985 |
| chr13 | 87557235 | 87557285 |
| chr13 | 93357611 | 93357661 |
| chr13 | 101519354 | 101519404 |
| chr13 | 103750266 | 103750316 |
| chr13 | 105229716 | 105229766 |
| chr13 | 109589028 | 109589078 |
| chr14 | 28192632 | 28192682 |
| chr14 | 28410691 | 28410741 |
| chr14 | 29504502 | 29504552 |
| chr14 | 40465885 | 40465935 |
| chr14 | 45303870 | 45303920 |
| chr14 | 45673413 | 45673463 |
| chr14 | 48863603 | 48863653 |
| chr14 | 62240269 | 62240319 |
| chr14 | 65028294 | 65028344 |
| chr14 | 65189705 | 65189755 |
| chr14 | 77515500 | 77515550 |
| chr14 | 83969844 | 83969894 |
| chr14 | 86225265 | 86225315 |
| chr14 | 87675900 | 87675950 |
| chr14 | 89250952 | 89251002 |
| chr14 | 90125343 | 90125393 |
| chr14 | 95947060 | 95947110 |
| chr14 | 101651173 | 101651223 |
| chr14 | 102907347 | 102907397 |
| chr15 | 20536796 | 20536846 |
| chr15 | 23852540 | 23852590 |
| chr15 | 35206036 | 35206086 |
| chr15 | 37287148 | 37287198 |
| chr15 | 40505373 | 40505423 |
| chr15 | 42425918 | 42425968 |
| chr15 | 44348022 | 44348072 |
| chr15 | 50549867 | 50549917 |
| chr15 | 87300995 | 87301045 |
| chr15 | 89746193 | 89746243 |
| chr15 | 98153048 | 98153098 |
| chr15 | 98477171 | 98477221 |
| chr15 | 98692502 | 98692552 |
| chr16 | 737137 | 737187 |
| chr16 | 4150930 | 4150980 |
| chr16 | 6861161 | 6861211 |
| chr16 | 10199562 | 10199612 |
| chr16 | 10821743 | 10821793 |
| chr16 | 10821691 | 10821741 |
| chr16 | 13721231 | 13721281 |
| chr16 | 22636284 | 22636334 |
| chr16 | 24006986 | 24007036 |
| chr16 | 54127369 | 54127419 |
| chr16 | 57534152 | 57534202 |
| chr16 | 60071023 | 60071073 |
| chr16 | 60338285 | 60338335 |
| chr16 | 62308324 | 62308374 |
| chr16 | 65386492 | 65386542 |
| chr16 | 66686535 | 66686585 |
| chr16 | 74677251 | 74677301 |
| chr16 | 76617176 | 76617226 |
| chr16 | 77472991 | 77473041 |
| chr16 | 86414160 | 86414210 |
| chr16 | 86686199 | 86686249 |
| chr16 | 89436254 | 89436304 |
| chr16 | 89438836 | 89438886 |
| chr17 | 3035453 | 3035503 |
| chr17 | 13913677 | 13913727 |

Table 15EI. Repeat Category U1

| | | |
|---|---|---|
| chr17 | 47501447 | 47501497 |
| chr17 | 57218909 | 57218959 |
| chr17 | 65954059 | 65954109 |
| chr18 | 3413958 | 3414008 |
| chr19 | 11410623 | 11410673 |
| chr19 | 44231592 | 44231642 |
| chr19 | 52143735 | 52143785 |
| chr1 | 6988998 | 6989048 |
| chr1 | 16221591 | 16221641 |
| chr1 | 16603248 | 16603298 |
| chr1 | 24344600 | 24344650 |
| chr1 | 28521410 | 28521460 |
| chr1 | 45237333 | 45237383 |
| chr1 | 108864439 | 108864489 |
| chr1 | 142425044 | 142425094 |
| chr1 | 143131391 | 143131441 |
| chr1 | 143674498 | 143674548 |
| chr1 | 143698675 | 143698725 |
| chr1 | 143820974 | 143821024 |
| chr1 | 144063380 | 144063430 |
| chr1 | 146129449 | 146129499 |
| chr1 | 221338206 | 221338256 |
| chr20 | 49242053 | 49242103 |
| chr21 | 41910493 | 41910543 |
| chr22 | 16498997 | 16499047 |
| chr22 | 39554301 | 39554351 |
| chrX | 2325388 | 2325438 |
| chrX | 117311925 | 117311975 |
| chr2 | 97131960 | 97132010 |
| chr2 | 100815232 | 100815282 |
| chr2 | 130277949 | 130277999 |
| chr2 | 219396064 | 219396114 |
| chr2 | 233746072 | 233746122 |
| chr3 | 72542783 | 72542833 |
| chr3 | 123790004 | 123790054 |
| chr3 | 126486150 | 126486200 |
| chr3 | 133320736 | 133320786 |
| chr5 | 137527124 | 137527174 |
| chr5 | 170918705 | 170918755 |
| chr6 | 4021635 | 4021685 |
| chr6 | 47838362 | 47838412 |
| chr6 | 89419682 | 89419732 |
| chr6 | 108352806 | 108352856 |
| chr6 | 108352726 | 108352776 |
| chr7 | 5269210 | 5269260 |
| chr7 | 29694750 | 29694800 |
| chr7 | 65311172 | 65311222 |
| chr7 | 137276851 | 137276901 |
| chr7 | 151684647 | 151684697 |
| chr11 | 122468037 | 122468087 |
| chr9 | 131244434 | 131244484 |
| chr9 | 135375222 | 135375272 |
| chr12 | 87477986 | 87478036 |
| chr12 | 129653009 | 129653059 |
| chr14 | 100815275 | 100815325 |
| chr15 | 80182352 | 80182402 |
| chr10 | 69817367 | 69817417 |
| chr16 | 11624719 | 11624769 |
| chr16 | 67200540 | 67200590 |
| chr17 | 1726256 | 1726306 |
| chr17 | 7715237 | 7715287 |

### REFERENCES

Agresti, Alan. (2002). Categorical Data Analysis. New York: Wiley-Interscience. ISBN 0-471-36093-7.
Alves, P. M., Lévy, N., Stevenson, B. J., Bouzourene, H., Theiler, G., Bricard, G., Viatte, S., Ayyoub, M., Vuilleumier, H., Givel, J. C., Rimoldi, D., Speiser, D. E., Jongeneel, C. V., Romero, P. J. and Lévy, F.: Identification of tumor-associated antigens by large-scale analysis of genes expressed in human colorectal cancer. Cancer Immun 8 (2008) 11.
Aravin A A, Sachidanandam R, Bourc'his D, Schaefer C, Pezic D, Toth K F, Bestor T, Hannon G J.: A piRNA pathway primed by individual transposons is linked to de novo DNA methylation in mice. Mol Cell. 31 (2008) 785-799.
Aravin A A, Sachidanandam R, Girard A, Fejes-Toth K, Hannon G J.: Developmentally regulated piRNA clusters implicate MILI in transposon control. Science. 316 (2007) 744-747.
Babushok, D. V. and Kazazian, H. H.: Progress in understanding the biology of the human mutagen LINE-1. Hum Mutat 28 (2007) 527-539.
Batzer, M. A. and Deininger, P. L.: Alu repeats and human genomic diversity. Nat Rev Genet 3 (2002) 370-379.
Benson, G.: Tandem repeats finder: a program to analyze DNA sequences. Nucleic Acids Research 27 (1999) 573-580.
Breiman, L. (2001). “Random Forests”. Machine Learning 45 (1): 5-32; doi:10.1023/A:1010933404324.
Brennecke, J., Aravin, A. A., Stark, A., Dus, M., Kellis, M., Sachidanandam, R. and Hannon, G. J.: Discrete small RNA-generating loci as master regulators of transposon activity in Drosophila. Cell 128 (2007) 1089-1103.
Büscher, K., Hahn, S., Hofmann, M., Trefzer, U., Ozel, M., Sterry, W., Löwer, J., Löwer, R., Kurth, R. and Denner, J.: Expression of the human endogenous retrovirus-K transmembrane envelope, Rec and Np9 proteins in melanomas and melanoma cell lines. Melanoma Res 16 (2006) 223-234.
Carter, A. B., Salem, A. H., Hedges, D. J., Keegan, C. N., Kimball, B., Walker, J. A., Watkins, W. S., Jorde, L. B. and Batzer, M. A.: Genome-wide analysis of the human Alu Yb-lineage. Hum Genomics 1 (2004) 167-178.
CGP, Cancer Genome Project (2009) available at website sanger.ac.uk/genetics/CGP/.
Chalitchagorn et al. Distinctive pattern of LINE-1 methylation level in normal tissues and the association with carcinogenesis. Oncogene (2004) vol. 23 (54) pp. 8841-6.
Chalitchagorn K, Shuangshoti S, Hourpai N, Kongruttanachok N, Tangkijvanich P, Thong-ngam D, Voravud N, Sriuranpong V, Mutirangura A. Distinctive pattern of LINE-1 methylation level in normal tissues and the association with carcinogenesis. Oncogene. 2004 Nov. 18; 23(54):8841-6.
Choi J Y, James S R, Link P A, McCann S E, Hong C C, Davis W, Nesline M K, Ambrosone C B, Karpf A R. Association between global DNA hypomethylation in leukocytes and risk of breast cancer. Carcinogenesis. 2009 Jul. 7.
Clarke J, Wu H C, Jayasinghe L, Patel A, Reid S, Bayley H. Continuous base identification for single-molecule nanopore DNA sequencing. Nat Nanotechnol. 4:265-70. 2009.
Dean F B, Hosono S, Fang L, Wu X, Faruqi A F, Bray-Ward P, Sun Z, Zong Q, Du Y, Du J, Driscoll M, Song W, Kingsmore S F, Egholm M, Lasken R S. (2002) Comprehensive human genome amplification using multiple displacement amplification. Proc Natl Acad Sci USA. 99:5261-6.
Dixon J M, Lubomirski M, Amaratunga D, Morrison T B, Brenan C J, Ilyin S E., Nanoliter high-throughput RT-qPCR: a statistical analysis and assessment. Biotechniques. 46:ii-viii., 2009.
Eickbush, T. H.: Repair by retrotransposition. Nature Genet. 31 (2002) 126-127.
Florl, A. R., Löwer, R., Schmitz-Dräger, B. J. and Schulz, W. A.: DNA methylation and expression of LINE-1 and HERV-K provirus sequences in urothelial and renal cell carcinomas. Br J Cancer 80 (1999) 1312-1321.
Flusberg B A, Webster D R, Lee J H, Travers K J, Olivares E C, Clark T A, Korlach J, Turner S W., Direct detection of DNA methylation during single-molecule, real-time sequencing. Nat Methods. 7:461-465. 2010.
Georgiou I, Noutsopoulos D, Dimitriadou E, Markopoulos G, Apergi A, Lazaros L, Vaxevanoglou T, Pantos K, Syrrou M, Tzavaras T.: Retrotransposon RNA expression and evidence for retrotransposition events in human oocytes. Hum Mol Genet. 18 (2009) 1221-1228.
Gnirke A, Melnikov A, Maguire J, Rogov P, LeProust E M, Brockman W, Fennell T, Giannoukos G, Fisher S, Russ C, Gabriel S, Jaffe D B, Lander E S, Nusbaum C., Solution hybrid selection with ultra-long oligonucleotides for massively parallel targeted sequencing. Nat Biotechnology 27:182-189. 2009.
Golan, M., Hizi, A., Resau, J. H., Yaal-Hahoshen, N., Reichman, H., Keydar, I. and Tsarfaty, I.: Human endogenous retrovirus (HERV-K) reverse transcriptase as a breast cancer prognostic marker. Neoplasia 10 (2008) 521-533.
Griffiths-Jones, S.: miRBase: the microRNA sequence database. Methods Mol Biol (2006).
Guttman M, Amit I, Garber M, French C, Lin M F, Feldser D, Huarte M, Zuk O, Carey B W, Cassady J P, Cabili M N, Jaenisch R, Mikkelsen T S, Jacks T, Hacohen N, Bernstein B E, Kellis M, Regev A, Rinn J L, Lander E S.: Chromatin signature reveals over a thousand highly conserved large non-coding RNAs in mammals. Nature. 458 (2009) 223-227.
Guyon, I. et al. (2002) Gene selection for cancer classification using support vector machines. Mach. Learn., 46, 389-422.
H. A. Chipman, E. I. George, and R. E. McCulloch. BART: Bayesian Additive Regression Trees. Journaβl of the Royal Statistical Society, 2006. Ser. B.
Healy, J., Thomas, E., Schwartz, J. and Wigler, M.: Annotating large genomes with exact word matches. Genome Res 13 (2003) 2306-2315.
Helleday, T., Lo, J., van Gent, D. C. and Engelward, B. P.: DNA double-strand break repair: from mechanistic understanding to cancer treatment. DNA Repair (Amst) 6 (2007) 923-935.
Herbst, H., Sauter, M., Kühler-Obbarius, C., Löning, T. and Mueller-Lantzsch, N.: Human endogenous retrovirus (HERV)-K transcripts in germ cell and trophoblastic tumours. APMIS 106 (1998) 216-220.
Hsiung et al. Global DNA methylation level in whole blood as a biomarker in head and neck squamous cell carcinoma. Cancer epidemiology, biomarkers & prevention: a publication of the American Association for Cancer Research, cosponsored by the American Society of Preventive Oncology (2007) vol. 16 (1) pp. 108-14.
Ince T A, Richardson A L, Bell G W, Saitoh M, Godar S, Karnoub A E, Iglehart J D, Weinberg R A. Transformation of different human breast epithelial cell types leads to distinct tumor phenotypes. Cancer Cell. 2007 August; 12(2):160-70.
J. P. Marques de Sa. Pattern Recognition: Concepts, Methods and Applications. Springer, 2001.
Jurka, J.: A new subfamily of recently retroposed human Alu repeats. Nucleic Acids Research 21 (1993) 2252.
Jurka, J.: Repeats in genomic DNA: mining and meaning. Curr Opin Struct Biol 8 (1998) 333-337.
Kapitonov, V. and Jurka, J.: The age of Alu subfamilies. J Mol Evol 42 (1996) 59-65.
Kent, W. J.: BLAT—the BLAST-like alignment tool. Genome Res 12 (2002) 656-664.
Khan, H., Smit, A. and Boissinot, S.: Molecular evolution and tempo of amplification of human LINE-1 retrotransposons since the origin of primates. Genome Res 16 (2006) 78-87.
Korshunova Y, Maloney R K, Lakey N, Citek R W, Bacher B, Budiman A, Ordway J M, McCombie W R, Leon J, Jeddeloh J A, McPherson J D. Massively parallel bisulphite pyrosequencing reveals the molecular complexity of breast cancer-associated cytosine-methylation patterns obtained from tissue and serum DNA. Genome Res. 2008 January; 18(1):19-29.
Kuramochi-Miyagawa, S., Watanabe, T., Gotoh, K., Totoki, Y., Toyoda, A., Ikawa, M., Asada, N., Kojima, K., Yamaguchi, Y., Ijiri, T. W., Hata, K., L1, E., Matsuda, Y., Kimura, T., Okabe, M., Sakaki, Y., Sasaki, H. and Nakano, T.: DNA methylation of retrotransposon genes is regulated by Piwi family members MILI and MIWI2 in murine fetal testes. Genes Dev 22 (2008) 908-917.
L. Breiman, J. Friedman, C. J. Stone, and R. A. Olshen. Classification and Regression Trees. Chapman & Hall/CRC, 1984.
Lage J M, Leamon J H, Pejovic T, Hamann S, Lacey M, Dillon D, Segraves R, Vossbrinck B, González A, Pinkel D, Albertson D G, Costa J, Lizardi P M. Whole genome analysis of genetic alterations in small DNA samples using hyperbranched strand displacement amplification and array-CGH.: Genome Res. (2003) 13:294-307.
Lage, J. M., and Lizardi P. M. “Introduction to Whole genome Amplification” in Whole Genome Amplification: Methods Express, edited by S. Hughes and R. Lasken, Scion Publishing Limited, 2005.
Lin, S. and Ying, S.: Gene silencing in vitro and in vivo using intronic microRNAs. Methods Mol Biol 342 (2006) 295-312.
Löwer, R., Löwer, J., Frank, H., Harzmann, R. and Kurth, R.: Human teratocarcinomas cultured in vitro produce unique retrovirus-like viruses. J Gen Virol 65 (1984) 887-898.
Macfarlane, C. and Simmonds, P.: Allelic variation of HERV-K(HML-2) endogenous retroviral elements in human populations. J Mol Evol 59 (2004) 642-656.
Mandal, P. K. and Kazazian, H. H.: SnapShot: Vertebrate transposons. Cell 135 (2008) 192-192.e1.
McLachlan, G. J. (2004). Discriminant Analysis and Statistical Pattern Recognition. Wiley Interscience.
Menendez, L., Benigno, B. B. and McDonald, J. F.: L1 and HERV-W retrotransposons are hypomethylated in human ovarian carcinomas. Molecular Cancer 3 (2004) 12.
Morrish, T. A., Gilbert, N., Myers, J. S., Vincent, B. J., Stamato, T. D., Taccioli, G. E., Batzer, M. A. and Moran, J. V.: DNA repair mediated by endonuclease-independent LINE-1 retrotransposition. Nat Genet 31 (2002) 159-165.
Morrison T, Hurley J, Garcia J, Yoder K, Katz A, Roberts D, Cho J, Kanigan T, Ilyin S E, Horowitz D, Dixon J M, Brenan C J. Nanoliter high throughput quantitative PCR. Nucleic Acids Res. 34:e123, 2006.
Muster, T., Waltenberger, A., Grassauer, A., Hirschl, S., Caucig, P., Romirer, I., Fodinger, D., Seppele, H., Schanab, O., Magin-Lachmann, C., Löwer, R., Jansen, B., Pehamberger, H. and Wolff, K.: An endogenous retrovirus derived from human melanoma cells. Cancer Res 63 (2003) 8735-8741.
Myers S, Freeman C, Auton A, Donnelly P, McVean G. A common sequence motif associated with recombination hot spots and genome instability in humans.: Nat Genet. 2008 40:1124-9.
Myers, S., Bottolo, L., Freeman, C., McVean, G. and Donnelly, P.: A fine-scale map of recombination rates and hotspots across the human genome. Science 310 (2005) 321-324.
Nakahara, T., Okuzawa M., Maeda, H., Hirano, M., T. Matsumoto, T., and H. Uchimura, H., Simultaneous Determination of Purine and Pyrimidine Bases Using High-Performance Liquid Chromatography with Electrochemical Detection: Application to DNA Assay, Journal of Liquid Chromatography & Related Technologies, Volume 15, Issue 10 Jul. 1992, pages 1785-1796.
Nigumann P, Redik K, Mälik K, Speek M. Many human genes are transcribed from the antisense promoter of L1 retrotransposon. Genomics 79 (2002) 628-634.
Oricchio, E., Sciamanna, I., Beraldi, R., Tolstonog, G., Schumann, G. and Spadafora, C.: Distinct roles for LINE-1 and HERV-K retroelements in cell proliferation, differentiation and tumor progression. Oncogene 26 (2007) 4226-4233.
Pace, J. K. and Feschotte, C.: The evolutionary history of human DNA transposons: evidence for intense activity in the primate lineage. Genome Res 17 (2007) 422-432.
Patzke, S., Lindeskog, M., Munthe, E. and Aasheim, H. C.: Characterization of a novel human endogenous retrovirus, HERV-H/F, expressed in human leukemia cell lines. Virology 303 (2002) 164-173.
Penzkofer, T., Dandekar, T. and Zemojtel, T.: L1Base: from functional annotation to prediction of active LINE-1 elements. Nucleic Acids Research 33 (2005) D498-500.
Rago C, Huso D L, Diehl F, Karim B, Liu G, Papadopoulos N, Samuels Y, Velculescu V E, Vogelstein B, Kinzler K W, Diaz L A Jr. Serial assessment of human tumor burdens in mice by the analysis of circulating DNA. Cancer Res. 2007 Oct. 1; 67(19):9364-70.
Reiss, D. and Mager, D. L.: Stochastic epigenetic silencing of retrotransposons: does stability come with age? Gene 390 (2007) 130-135.
Rockwood L. D., Felix, K. and Janz, S.: Elevated presence of retrotransposons at sites of DNA double strand break repair in mouse models of metabolic oxidative stress and MYC-induced lymphoma. Mutat Res. 548 (2004) 117-125.
Rodriguez J, Vives L, Jordà M, Morales C, Muñoz M, Vendrell E, Peinado M A.: Genome-wide tracking of unmethylated DNA Alu repeats in normal and cancer cells. Nucleic Acids Res. 36 (2008) 770-784.
Roman-Gomez, J., Jimenez-Velasco, A., Agirre, X., Cervantes, F., Sanchez, J., Garate, L., Barrios, M., Castillejo, J. A., Navarro, G., Colomer, D., Prosper, F., Heiniger, A. and Torres, A.: Promoter hypomethylation of the LINE-1 retrotransposable elements activates sense/antisense transcription and marks the progression of chronic myeloid leukemia. Oncogene 24 (2005) 7213-7223.
Romano, C. M., Ramalho, R. F. and Zanotto, P. M.: Tempo and mode of ERV-K evolution in human and chimpanzee genomes. Arch Virol 151 (2006) 2215-2228.
Sen, S. K., Huang, C. T., Han, K. and Batzer, M. A.: Endonuclease-independent insertion provides an alternative pathway for L1 retrotransposition in the human genome. Nucleic Acids Research 35 (2007) 3741-3751.
Serafino, A., Balestrieri, E., Pierimarchi, P., Matteucci, C., Moroni, G., Oricchio, E., Rasi, G., Mastino, A., Spadafora, C., Garaci, E. and Vallebona, P. S.: The activation of human endogenous retrovirus K (HERV-K) is implicated in melanoma cell malignant transformation. Exp Cell Res 315 (2009) 849-862.
Sinibaldi-Vallebona, P., Lavia, P., Garaci, E. and Spadafora, C.: A role for endogenous reverse transcriptase in tumorigenesis and as a target in differentiating cancer therapy. Genes Chromosom. Cancer 45 (2005) 1-10.
Smit, A. F., Hubley, R. and Green, P.: RepeatMasker Open-3.0. (1996-2004) available at website repeatmasker.org.
Smit, A. F.: Identification of a new, abundant superfamily of mammalian LTR-transposons. Nucleic Acids Research 21 (1993) 1863-1872.
Smith, I. M., Mydlarz, W. K., Mithani, S. K. and Califano, J. A.: DNA global hypomethylation in squamous cell head and neck cancer associated with smoking, alcohol consumption and stage. Int J Cancer 121 (2007) 1724-1728.
Soørensen A L, Collas P. Immunoprecipitation of methylated DNA. Methods Mol Biol. 567:249-262. 2009.
Speek M.: Antisense promoter of human L1 retrotransposon drives transcription of adjacent cellular genes. Mol Cell Biol. 21 (2001) 1973-1985.
Srikanta, D., Sen, S. K., Huang, C. T., Conlin, E. M., Rhodes, R. M. and Batzer, M. A.: An alternative pathway for Alu retrotransposition suggests a role in DNA double-strand break repair. Genomics 93 (2009) 205-212.
Stauffer, Y., Theiler, G., Sperisen, P., Lebedev, Y. and Jongeneel, C. V.: Digital expression profiles of human endogenous retroviral families in normal and cancerous tissues. Cancer Immun 4 (2004) 2.
Strobl C, Malley J, Tutz G. An introduction to recursive partitioning: rationale, application, and characteristics of classification and regression trees, bagging, and random forests. Psychol Methods. 14:323-48. 2009.
Sunami E, Vu A T, Nguyen S L, Giuliano A E, Hoon D S. Quantification of LINE1 in circulating DNA as a molecular biomarker of breast cancer. Ann NY Acad Sci. 2008 August; 1137:171-4.
Suter C M, Martin D I, Ward R L.: Hypomethylation of L1 retrotransposons in colorectal cancer and adjacent normal tissue. Int J Colorectal Dis. 19 (2004) 95-101.
Szpakowski S, Sun X, Lage J M, Dyer A, Rubinstein J, Kowalski D, Sasaki C, Costa J, Lizardi P M. Loss of epigenetic silencing in tumors preferentially affects primate-specific retroelements. Gene. 448:151-67. 2009.
Takai, D. and Jones, P.: Comprehensive analysis of CpG islands in human chromosomes 21 and 22. Proc Natl Acad Sci USA 99 (2002) 3740-3745.
Teneng, I., Stribinskis, V. and Ramos, K. S.: Context-specific regulation of LINE-1. Genes Cells 12 (2007) 1101-1110.
Tremblay, A., Jasin, M. and Chartrand, P.: A double-strand break in a chromosomal LINE element can be repaired by gene conversion with various endogenous LINE elements in mouse cells. Mol Cell Biol 20 (2000) 54-60.
Vapnik, V. N. (1998) Statistical Learning Theory. John Wiley and Sons, New York.
Wang, H., Xing, J., Grover, D., Hedges, D., Han, K., Walker, J. and Batzer, M.: SVA elements: a hominid-specific retroposon family. J Mol Biol 354 (2005) 994-1007.
Wang-Johanning, F., Frost, A. R., Jian, B., Azerou, R., Lu, D. W., Chen, D. T. and Johanning, G. L.: Detecting the expression of human endogenous retrovirus E envelope transcripts in human prostate adenocarcinoma. Cancer 98 (2003) 187-197.
Wang-Johanning, F., Frost, A. R., Johanning, G. L., Khazaeli, M. B., LoBuglio, A. F., Shaw, D. R. and Strong, T. V.: Expression of human endogenous retrovirus k envelope transcripts in human breast cancer. Clin Cancer Res 7 (2001) 1553-1560.
Wang-Johanning, F., Liu, J., Rycaj, K., Huang, M., Tsai, K., Rosen, D. G., Chen, D. T., Lu, D. W., Barnhart, K. F. and Johanning, G. L.: Expression of multiple human endogenous retrovirus surface envelope proteins in ovarian cancer. Int J Cancer 120 (2007) 81-90.
Woloszynska-Read et al. Intertumor and intratumor NY-ESO-1 expression heterogeneity is associated with promoter-specific and global DNA methylation status in ovarian cancer. Clinical cancer research: an official journal of the American Association for Cancer Research (2008) vol. 14 (11) pp. 3283-90.
Xing, J., Hedges, D., Han, K., Wang, H., Cordaux, R. and Batzer, M.: Alu element mutation spectra: molecular clocks and the effect of DNA methylation. J Mol Biol 344 (2004) 675-682.
Xing, J., Witherspoon, D. J., Ray, D. A., Batzer, M. A. and Jorde, L. B.: Mobile DNA elements in primate and human evolution. Am J Phys Anthropol Suppl 45 (2007) 2-19.
Yang A S, Estécio M R, Doshi K, Kondo Y, Tajara E H, Issa J P. A simple method for estimating global DNA methylation using bisulfite PCR of repetitive DNA elements. Nucleic Acids Res. 2004 Feb. 18; 32(3):e38.

1. A method of assessing one or more statuses of a subject, the method comprising:
determining the methylation state of one or more status biomarkers in the subject,
comparing one or more of the determined methylation states to one or more reference methylation states, wherein a difference, lack of a difference, or both in one or more of the determined methylation states and one or more of the reference methylation states indicates one or more statuses of the subject.

2. The method of claim 1, wherein the status biomarkers comprise nucleic acid sequences in the genome of the species to which the subject belongs, wherein the nucleic acid sequences are in proximity to CpG islands or islets, wherein the CpG islands or islets comprise nucleic acid regions greater than 100 nucleotides in length that contain a minimum of 5 CpG residues and have a ratio of CG content to GC content greater than 0.3.

3-8. (canceled)

9. The method of claim 2, wherein one or more of the status biomarkers comprises a probe binding site, wherein the probe binding site of the one or more of the status biomarkers is specific for a probe.

10. The method of claim 9, wherein one or more of the probes are specific for a repetitive DNA sequence locus, wherein the repetitive DNA sequence locus comprises one or more repetitive DNA sequences, wherein independently for each of the one or more of the probes one or more of the repetitive DNA sequences belongs to a family of repetitive DNA sequences listed in Table 1.

11. The method of claim 10, wherein each probe is specific for a repetitive DNA sequence locus, wherein independently for each probe one or more of the repetitive DNA sequences belongs to a family of repetitive DNA sequences listed in Table 1.

12-21. (canceled)

22. The method of claim 1, wherein the methylation state of more than 100 biomarkers is determined.

23-26. (canceled)

27. The method of claim 1, wherein a plurality of the biomarkers independently belong to one or more status biomarker families, wherein each biomarker in each status biomarker family comprises one or more repetitive DNA sequences that belong to a single family of repetitive DNA sequences listed in Table 1.

28-41. (canceled)

42. The method of claim 1, wherein the status biomarkers comprise a set of status biomarkers, wherein the members of the set of status biomarkers are status biomarkers that indicate the status of one or more specific statuses.

43. The method of claim 42, wherein the one or more specific statuses comprise wellness, level of health, risk to wellness, risk to level of health, status of the genome, genomic instability, aging, risk of aging, cancer, risk of cancer, head and neck cancer, risk of head and neck cancer, breast cancer, risk of breast cancer, lung cancer, risk of lung cancer, prostate cancer, risk of prostate cancer, colon cancer, risk of colon cancer, esophageal cancer, risk of esophageal cancer, ovarian cancer, risk of ovarian cancer, liver cancer, risk of liver cancer, pancreatic cancer, risk of pancreatic cancer, skin cancer, risk of skin cancer, melanoma, risk of melanoma, lymphoma, risk of lymphoma, leukemia, risk of leukemia, cervical cancer, risk of cervical cancer, cervical dysplasia, risk of cervical dysplasia, cervical intraepithelial neoplasia, risk of cervical intraepithelial neoplasia, tumor burden, stress response, diabetes, risk of diabetes, heart disease, risk of heart disease, and response to treatment.

44. (canceled)

45. The method of claim 1, wherein the one or more specific statuses comprise a lack of wellness, low level of health, risk to wellness, risk to level of health, poor status of the genome, genomic instability, aging, risk of aging, cancer, risk of cancer, head and neck cancer, risk of head and neck cancer, breast cancer, risk of breast cancer, lung cancer, risk of lung cancer, prostate cancer, risk of prostate cancer, colon cancer, risk of colon cancer, esophageal cancer, risk of esophageal cancer, ovarian cancer, risk of ovarian cancer, liver cancer, risk of liver cancer, pancreatic cancer, risk of pancreatic cancer, skin cancer, risk of skin cancer, melanoma, risk of melanoma, lymphoma, risk of lymphoma, leukemia, risk of leukemia, cervical cancer, risk of cervical cancer, cervical dysplasia, risk of cervical dysplasia, cervical intraepithelial neoplasia, risk of cervical intraepithelial neoplasia, tumor burden, stress response, diabetes, risk of diabetes, heart disease, and risk of heart disease.

46. The method of claim 1, wherein the methylation state is determined by
treating a DNA sample of the subject to differentiate methylated and unmethylated nucleotides,
detecting the level of methylated forms of the one or more status biomarkers in the treated DNA, detecting the level of unmethylated forms of the one or more status biomarkers in the treated DNA, or both, wherein the level of methylated forms of the status biomarkers, the level of unmethylated forms of the status biomarkers, or both indicates the methylation state of the status biomarkers.

47. The method of claim 46, wherein treating the DNA sample is accomplished by incubating the DNA sample with one or more restriction endonucleases and amplifying the incubated DNA, wherein the restriction endonucleases are methylation-sensitive restriction endonucleases, wherein the level of the status biomarkers in the amplified DNA is lower when the status biomarkers have reduced methylation and the level of the status biomarkers in the amplified DNA is higher when the status biomarkers have increased methylation, wherein the level of the status biomarkers comprise the level of methylated forms of the one or more status biomarkers in the treated DNA, the level of unmethylated forms of the one or more status biomarkers in the treated DNA, or both.

48-53. (canceled)

54. The method of claim 46, wherein treating the DNA sample is accomplished by processing the DNA sample with sodium bisulfite.

55. The method of claim 46, wherein treating the DNA sample is accomplished by fragmenting the DNA and separating methylated DNA from unmethylated DNA.

56. (canceled)

57. The method of claim 55, wherein methylated DNA is separated from unmethylated DNA by binding methylated DNA with a specific binding molecule specific for methyl groups and separating the bound form the unbound DNA.

58. (canceled)

59. The method of claim 46, wherein treating the DNA sample is accomplished by capturing status biomarker DNA fragments and sequencing the captured status biomarker DNA fragments, wherein the sequencing distinguishes cytosine from methylcytosine, wherein the level of methylcytosine indicates level of methylated forms of the status biomarkers.

60-68. (canceled)

69. The method of claim 59 further comprising, after capturing status biomarker DNA fragments and prior to sequencing the captured status biomarker DNA fragments, releasing the captured status biomarker DNA fragments and recapturing the released status biomarker DNA fragments.

70-76. (canceled)

77. The method of claim 46, wherein detecting the level of the status biomarkers is accomplished via an array of probes specific for the status biomarkers.

78. (canceled)

79. The method of claim 77, wherein detecting the level of the status biomarkers is accomplished via amplifying the processed DNA and determining the ratio of cytosine to thymidine in the amplified DNA and converting the ratio to the level of methylated forms of the status biomarkers.

80. (canceled)

81. The method of claim 46, wherein detecting the level of the status biomarkers is accomplished via PCR amplification of the status biomarkers using primers specific for the status biomarkers.

82-83. (canceled)

84. The method of claim 46, wherein the level of the status biomarkers are grouped into a plurality of status biomarker families, wherein the level of the status biomarkers in one or more of the families is analyzed, wherein the analyzed level of the status biomarkers in the one or more of the families indicates the methylation state of the status biomarkers in the family.

85-87. (canceled)

88. The method of claim 1, wherein the level of one or more of the status biomarkers is normalized to one or more of the reference methylation states.

89. The method of claim 27, wherein the level of one or more of the status biomarker families is normalized to one or more of the reference methylation states.

90. The method of claim 84, wherein the status biomarkers are grouped according to one or more repetitive DNA sequences that the status biomarkers comprise, wherein each biomarker in each status biomarker family comprises one or more repetitive DNA sequences that belong to a single family of repetitive DNA sequences listed in Table 1.

91-92. (canceled)

93. The method of claim 1, wherein one or more of the one or more reference methylation states is the methylation state of the same subject at a different time, the methylation state of the same subject at an earlier time, the methylation state of the same subject at a later time, or the methylation state of one or more normal cells, tissues, organs, or a combination of the same subject.

94. The method of claim 1, wherein one or more of the one or more reference methylation states is the methylation state from non-tumor adjacent tissue.

95. (canceled)

96. The method of claim 1 further comprising determining the genetic state of one or more status biomarkers,
comparing one or more of the determined genetic states to one or more reference genetic states, wherein a difference, lack of a difference, or both in one or more of the determined genetic states and one or more of the reference genetic states indicates one or more statuses of the subject.

97. The method of claim 46, wherein the source of one or more of the DNA samples is one or more tissues of the subject, organs of the subject, or both.

98-100. (canceled)

101. The method of claim 1, wherein the subject is assessed for the status of wellness, level of health, risk to wellness, risk to level of health, or a combination.

102-117. (canceled)

118. A set of one or more status biomarkers, wherein the status biomarkers comprise nucleic acid sequences in a genome, wherein the nucleic acid sequences are in proximity to CpG islands or islets, wherein the CpG islands or islets comprise nucleic acid regions greater than 100 nucleotides in length that contain a minimum of 5 CpG residues and have a ratio of CG content to GC content greater than 0.3.

119. The method of claim 118, wherein the CpG islands or islets comprise nucleic acid regions greater than 200 nucleotides in length.

120-125. (canceled)

126. The set of claim 118, wherein one or more of the status biomarkers comprises a probe binding site, wherein the probe binding site of the one or more of the status biomarkers is specific for a probe, wherein one or more of the probes are specific for a repetitive DNA sequence locus, wherein the repetitive DNA sequence locus comprises one or more repetitive DNA sequences, wherein independently for each of the one or more of the probes one or more of the repetitive DNA sequences belongs to a family of repetitive DNA sequences listed in Table 1.

127. The set of claim 126, wherein each probe is specific for a repetitive DNA sequence locus, wherein independently for each probe one or more of the repetitive DNA sequences belongs to a family of repetitive DNA sequences listed in Table 1.

128-162. (canceled)

163. A method of identifying status biomarkers associated with a status of a subject, the method comprising:
determining the methylation state of one or more status biomarkers in one or more DNA samples, wherein the DNA samples are from sources that are relevant to one or more specific statuses,
comparing one or more of the determined methylation states to one or more reference methylation states, wherein a difference in one or more of the determined methylation states and one or more of the reference methylation states indicates that the status biomarkers for which the difference in the methylation states is found is a status biomarker associated with one or more of the specific statuses.

164. The method of claim 163, wherein the status biomarkers comprise nucleic acid sequences in the genome of the species to which the subject belongs, wherein the nucleic acid sequences are in proximity to CpG islands or islets, wherein the CpG islands or islets comprise nucleic acid regions greater than 100 nucleotides in length that contain a minimum of 5 CpG residues and have a ratio of CG content to GC content greater than 0.3.

165-171. (canceled)

172. The method of claim 164, wherein one or more of the status biomarkers comprises a probe binding site, wherein the probe binding site of the one or more of the status biomarkers is specific for a probe, wherein one or more of the probes are specific for a repetitive DNA sequence locus, wherein the repetitive DNA sequence locus comprises one or more repetitive DNA sequences, wherein independently for each of the one or more of the probes one or more of the repetitive DNA sequences belongs to a family of repetitive DNA sequences listed in Table 1.

173. The method of claim 172, wherein each probe is specific for a repetitive DNA sequence locus, wherein independently for each probe one or more of the repetitive DNA sequences belongs to a family of repetitive DNA sequences listed in Table 1.

174-262. (canceled)

263. A method of producing status biomarker capture probes, the method comprising
selecting a subset of repetitive DNA sequence loci from a set of repetitive DNA sequence loci, wherein the repetitive DNA sequence loci in the set of repetitive DNA sequence loci belong to a single one of the families of repetitive DNA sequence listed in Table 1, wherein the subset of repetitive DNA sequence loci are selected by identifying those repetitive DNA sequence loci that comprise a repetitive DNA sequence belonging to one of the families of repetitive DNA sequences listed in Table 17,
generating a set of status biomarker capture probe sequences, wherein each status biomarker capture probe sequence in the set has a length of 50 bases or more, wherein each status biomarker capture probe represented in the set of status biomarker capture probe sequences can hybridize to at least 5% of the repetitive DNA sequence loci in the selected subset of repetitive DNA sequence loci,
synthesizing one or more status biomarker capture probes, wherein each status biomarker capture probe has the sequence of one of the status biomarker capture probe sequences.

264. The method of claim 263, wherein the repetitive DNA sequence loci in the set of repetitive DNA sequence loci belong to a single one of the families of repetitive DNA sequence LTR54B, MER11B, MER34B, LTR56, THE1B, HERV9, LTR14C, HERVFH21, LTR6B, LTR46, MLT1D, MER67D, HERVK11, LTR10B, HERVK22, MER6, MER66C, MLT1G1, MER4D, and MLTD2.

265. The method of claim 263 further comprising selecting one or more additional subsets of repetitive DNA sequence loci each from a different additional set of repetitive DNA sequence loci, generating one or more additional sets of status biomarker capture probe sequences each based on one of the one or more additional subsets, and synthesizing one or more additional status biomarker capture probes, wherein each additional status biomarker capture probe has the sequence of one of the additional status biomarker capture probe sequences,
wherein the repetitive DNA sequence loci in each additional set of repetitive DNA sequence loci independently belong to a different single one of the families of repetitive DNA sequence listed in Table 1, wherein the repetitive DNA sequence loci in the set of repetitive DNA sequence loci and in each additional set of repetitive DNA sequence loci belong to different families of repetitive DNA sequence.

266-274. (canceled)
